(19) United States
(12) Patent Application Publication
(10) Pub. No.: US 2023/0301176 A1
(43) Pub. Date: Sep. 21, 2023

(54) COMPOUND, MATERIAL FOR ORGANIC ELECTROLUMINESCENCE ELEMENTS, ORGANIC ELECTROLUMINESCENCE ELEMENT, AND ELECTRONIC DEVICE

(71) Applicants: IDEMITSU KOSAN CO.,LTD., Tokyo (JP); National University Corporation Tokai National Higher Education and Research System, Nagoya-shi (JP)

(72) Inventors: Tsukasa SAWATO, Chiba-shi (JP); Keita SEDA, Chiba-shi (JP); Tasuku HAKETA, Chiba-shi (JP); Yusuke TAKAHASHI, Chiba-shi (JP); Shota TANAKA, Chiba-shi (JP); Takuto FUKAMI, Chiba-shi (JP); Hideto ITO, Nagoya-shi (JP); Kenichiro ITAMI, Nagoya-shi (JP)

(73) Assignees: IDEMITSU KOSAN CO.,LTD., Tokyo (JP); National University Corporation Tokai National Higher Education and Research System, Nagoya-shi (JP)

(21) Appl. No.: 18/050,713

(22) Filed: Oct. 28, 2022

(30) Foreign Application Priority Data
Nov. 1, 2021 (JP) 2021-179018

(51) Int. Cl. H10K 85/60; C07C 211/57; C09K 11/06; C07D 307/91; C07C 211/61

(52) U.S. Cl. CPC H10K 85/633; C07C 211/57; C09K 11/06; C07D 307/91; H10K 85/636; C07C 211/61; H10K 50/156

(57) ABSTRACT

Provided are a compound that further improves the performance of an organic EL device, the organic electroluminescence device with more improved device performance, and electronic device including such an organic electroluminescence device, in which the compound is represented by the following formula (1):

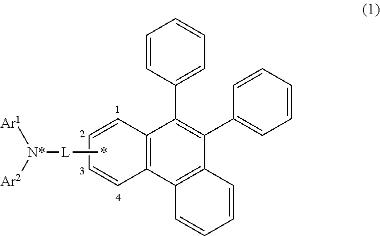

(each symbol in the formula (1) is as defined in the specification), the organic electroluminescence device contains the compound, and the electronic device includes the organic electroluminescence device.

### CROSS-REFERENCE TO RELATED APPLICATION

This application is based upon and claims the benefit of priority from the prior Japanese Patent Application No. 2021-179018, filed on Nov. 1, 2021; the entire contents of which are incorporated herein by reference.

### TECHNICAL FIELD

The present invention relates to a compound, a material for organic electroluminescence devices, the organic electroluminescence devices, and electronic device including the corresponding organic electroluminescence devices.

### BACKGROUND ART

In general, an organic electroluminescence device (hereinafter, also referred to as “an organic EL device”) is composed of an anode, a cathode, and organic layers interposed between the anode and the cathode. When a voltage is applied between both electrodes, electrons and holes are injected into a light-emitting region from a cathode side and an anode side, respectively. The injected electrons and holes recombine in the light-emitting region to cause an excited state, and emit light when the excited state returns to a ground state. Therefore, the development of a material that efficiently transports electrons or holes to a light-emitting region and facilitates recombination of the electrons and the holes is important in obtaining high-performance organic EL devices.

PTLs 1 to 3 disclose compounds used as materials for organic electroluminescence devices.

### CITATION LIST

### Patent Literature

[PTL 1] JP5703394B2
[PTL 2] US2019/0207117A1
[PTL 3] CN113105341A

### SUMMARY OF INVENTION

### Technical Problem

Although, many compounds for organic EL devices have conventionally been reported, there is still a need for compounds that further improve the performance of organic EL devices.

The present invention has been made to solve the above problems, and an object thereof is to provide a compound that further improves the performance of an organic EL device, an organic EL device having a further improved device performance, and electronic device including such an organic EL device.

The present inventors have repeatedly conducted intensive studies on the performance of organic EL devices including compounds described in PTLs 1 to 3. As a result, they have found that an organic EL device including a compound represented by the following formula (1) is more improved in performance.

In an aspect, the present invention provides a compound represented by the following formula (1).

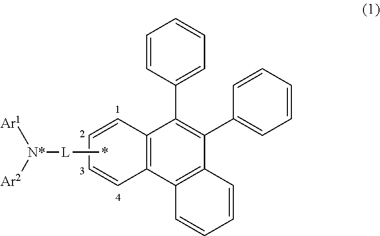

(in the formula, N* is a central nitrogen atom;
* is bonded to any of carbon atoms 1 to 4;
L is a single bond, an unsubstituted phenylene group, or an unsubstituted biphenylene group.
Ar¹ is a group represented by the following formula (1a), (1b), (1c), (1d), (1e), or (1f); and
Ar² is a group represented by the following formula (1b), (1c), (1d), (1e), (1f), or (1g).)

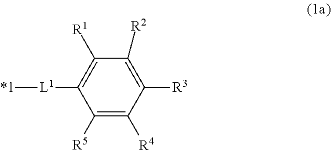

(in the formula,
each of R¹ to R⁵ is independently a hydrogen atom or a substituted or unsubstituted alkyl group having 1 to 6 carbon atoms;
adjacent two selected from R¹ to R⁵ are not bonded to each other, and thus do not form a ring;
L¹ is a single bond or an unsubstituted phenylene group; and
*1 is bonded to the central nitrogen atom N*.)

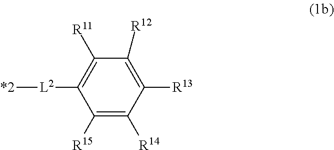

(in the formula,
each of R¹¹ to R¹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R¹¹ to R¹⁵ are not bonded to each other, and thus do not form a ring;
L² is an unsubstituted biphenylene group; and
*2 is bonded to the central nitrogen atom N*.)

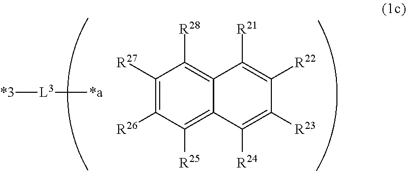

(in the formula,
each of R²¹ to R²⁸ is independently a hydrogen atom, a substituted or unsubstituted alkyl group having 1 to 6 carbon atoms, or a substituted or unsubstituted aryl group having 6 to 12 ring carbon atoms;
provided that one selected from R²¹ to R²⁸ is a single bond bonded to *a, and adjacent two selected from R²¹ to R²⁸ except for the single bond are not bonded to each other, and thus do not form a ring structure;
L³ is a single bond or an unsubstituted arylene group having 6 to 12 ring carbon atoms; and
*3 is bonded to the central nitrogen atom N*.)

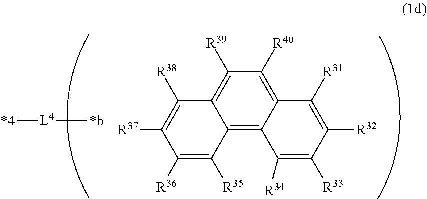

(in the formula,
each of R³¹ to R⁴⁰ is independently a hydrogen atom, a substituted or unsubstituted alkyl group having 1 to 6 carbon atoms, or a substituted or unsubstituted aryl group having 6 to 12 ring carbon atoms;
provided that one selected from R³¹ to R⁴⁰ is a single bond bonded to *b, and adjacent two selected from R³¹ to R⁴⁰ except for the single bond are not bonded to each other, and thus do not form a ring structure;
L⁴ is a single bond or an unsubstituted arylene group having 6 to 12 ring carbon atoms; and
*4 is bonded to the central nitrogen atom N*.)

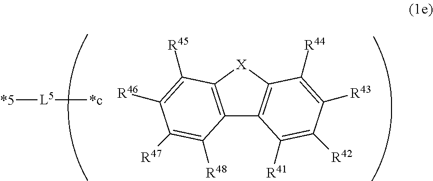

(in the formula,
X is an oxygen atom, a sulfur atom, NRa, or CRbRc;
Ra is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
Rb is a substituted or unsubstituted aryl group having 6 to 12 ring carbon atoms;
Rc is a substituted or unsubstituted alkyl group having 1 to 6 carbon atoms or a substituted or unsubstituted aryl group having 6 to 12 ring carbon atoms;
Rb and Rc may be bonded to each other to form a spiro ring structure;
each of R⁴¹ to R⁴⁸ is independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
provided that one selected from R⁴¹ to R⁴⁸ and Ra is a single bond bonded to *c;
adjacent two selected from R⁴¹ to R⁴⁸ except for the single bond may be bonded to each other to form one or more substituted or unsubstituted benzene rings. Meanwhile, when X is CRbRc, the adjacent two do not form a ring;
L⁵ is a single bond or an unsubstituted arylene group having 6 to 12 ring carbon atoms; and
*5 is bonded to the central nitrogen atom N*.)

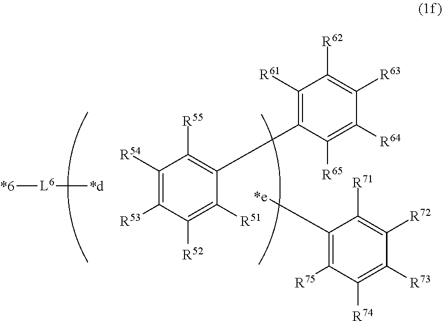

(in the formula,
each of R⁵¹ to R⁵⁵ is independently a hydrogen atom, an unsubstituted alkyl group having 1 to 6 carbon atoms, or an unsubstituted phenyl group;
provided that one selected from R⁵¹ to R⁵⁵ is a single bond bonded to *d, and another selected from R⁵¹ to R⁵⁵ is a single bond bonded to *e;
except for the single bond bonded to *d and the single bond bonded to *e, adjacent two selected from R⁵¹ to R⁵⁵ are not bonded to each other, and thus do not form a ring structure;
each of R⁶¹ to R⁶⁵ and R⁷¹ to R⁷⁵ is independently a hydrogen atom or an unsubstituted alkyl group having 1 to 6 carbon atoms;
adjacent two selected from R⁶¹ to R⁶⁵ and R⁷¹ to R⁷⁵ may be bonded to each other to form one or more substituted or unsubstituted benzene rings;
L⁶ is a single bond or an unsubstituted arylene group having 6 to 12 ring carbon atoms; and
*6 is bonded to the central nitrogen atom N*.)

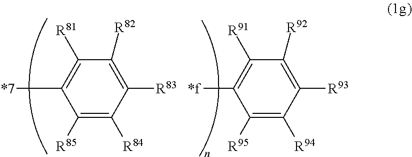

(in the formula,
each of R⁸¹ to R⁸⁵ is independently a hydrogen atom or a substituted or unsubstituted alkyl group having 1 to 6 carbon atoms;
provided that one selected from R⁸¹, R⁸², R⁸⁴, and R⁸⁵ is a single bond bonded to *f;
adjacent two selected from R⁸³ and R⁸¹, R⁸², R⁸⁴, and R⁸⁵ except for the single bond bonded to *f are not bonded to each other, and thus do not form a ring structure;
each of R⁹¹ to R⁹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R⁹¹ to R⁹⁵ are not bonded to each other, and thus do not form a ring structure;
n is 0 or 1, and then when n is 0, the formula (1g) indicates the following formula (1g′); and

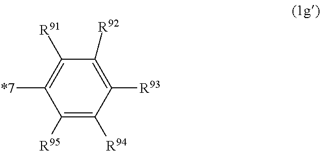

*7 in the formulas (1g) and (1g′) is bonded to the central nitrogen atom.)

In another aspect, the present invention provides a material for an organic EL device, which contains the compound represented by the formula (1).

In yet another aspect, the present invention provides an organic electroluminescence device including a cathode, an anode, and organic layers between the corresponding cathode and the corresponding anode. In the organic electroluminescence device, the corresponding organic layers include a light emitting layer, and at least one layer of the corresponding organic layers contains the compound represented by the formula (1).

In still another aspect, the present invention provides an electronic device including the organic electroluminescence device.

The organic EL device containing the compound represented by the formula (1) exhibits improved device performance.

### BRIEF DESCRIPTION OF DRAWINGS

FIG. 1 is a schematic view illustrating an example of the layer structure of an organic EL device according to an aspect of the present invention.

FIG. 2 is a schematic view illustrating another example of the layer structure of the organic EL device according to an aspect of the present invention.

FIG. 3 is a schematic view illustrating a further example of the layer structure of the organic EL device according to an aspect of the present invention.

### DESCRIPTION OF EMBODIMENTS

### Definitions

In the description herein, the hydrogen atom encompasses isotopes thereof having different numbers of neutrons, i.e., protium, deuterium, and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “unsubstituted” in the expression “BB group substituted by an AA group” similarly means that one or more hydrogen atom in the BB group is substituted by the AA group.

Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

Unsubstituted Aryl Group (Set of Specific Examples G1A):
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

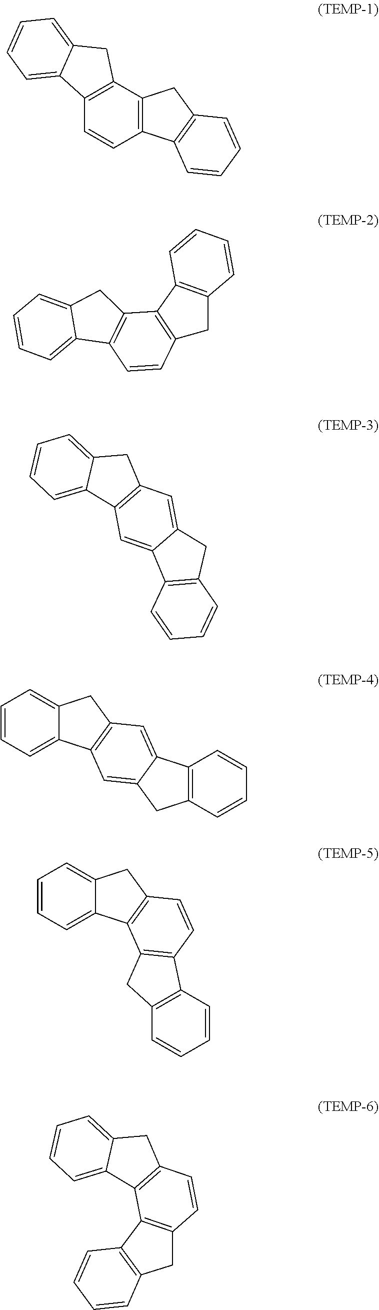

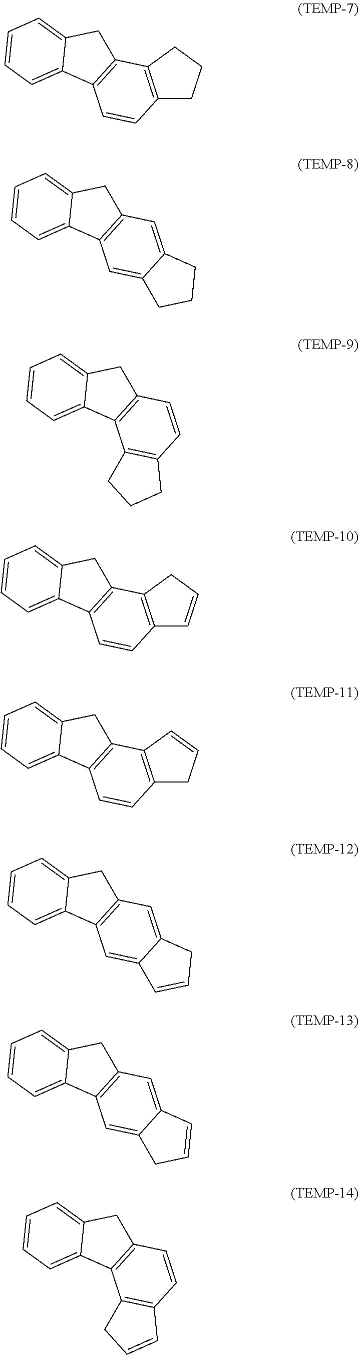

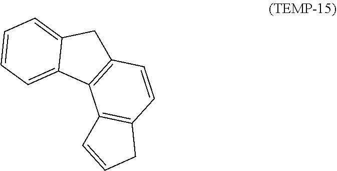

Substituted Aryl Group (Set of Specific Examples G1B):
an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

Unsubstituted Heterocyclic Group Containing Nitrogen Atom (Set of Specific Examples G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

Unsubstituted Heterocyclic Group Containing Oxygen Atom (Set of Specific Examples G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

Unsubstituted Heterocyclic Group Containing Sulfur Atom (Set of Specific Examples G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a nap hthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent Heterocyclic Group derived by removing One Hydrogen Atom from Ring Structures represented by General Formulae (TEMP-16) to (TEMP-33) (Set of Specific Examples G2A4)

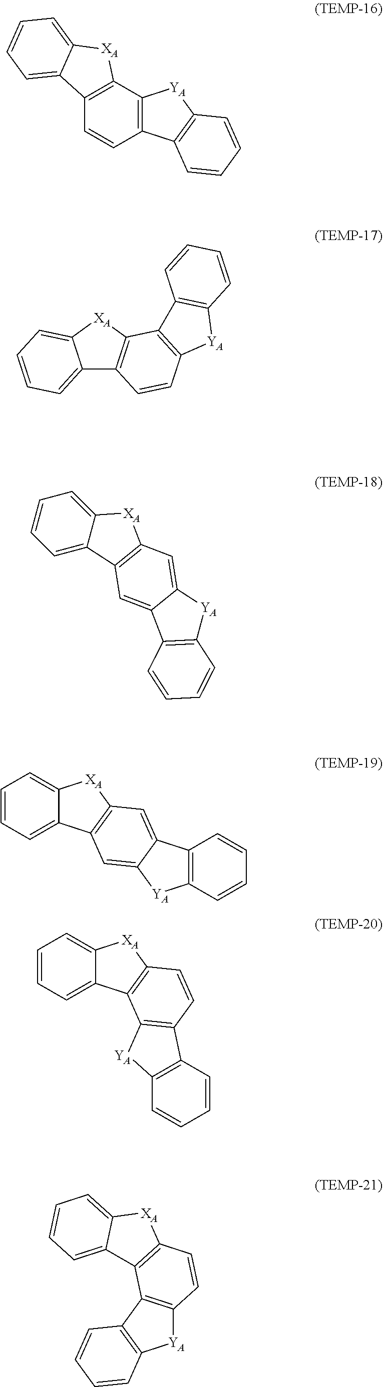

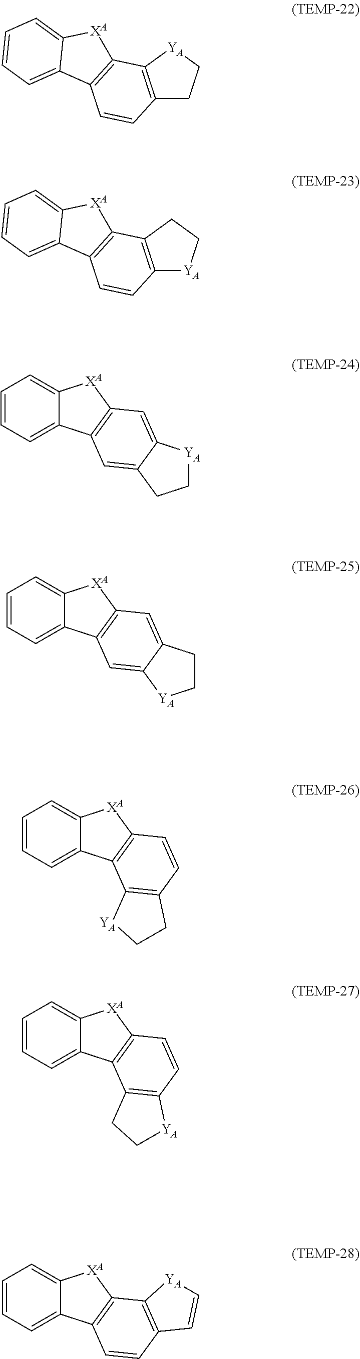

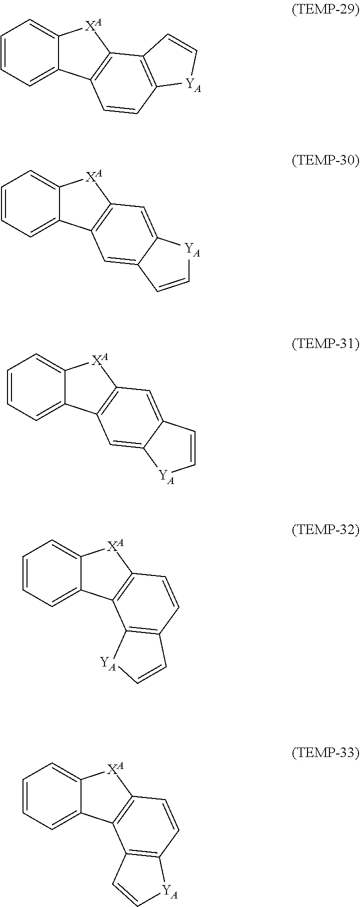

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

Substituted Heterocyclic Group Containing Nitrogen Atom (Set of Specific Examples G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.

Substituted Heterocyclic Group Containing Oxygen Atom (Set of Specific Examples G2B2):
a phenyldibenzofuranyl group,
a methyldibenzofuranyl group,
a t-butyldibenzofuranyl group, and
a monovalent residual group of spiro[9H-xanthene-9,9′-[9H]fluorene].

Substituted Heterocyclic Group Containing Sulfur Atom (Set of Specific Examples G2B3):
a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

Unsubstituted Alkyl Group (Set of Specific Examples G3A):
a methyl group,
an ethyl group,
a n-propyl group,
an isopropyl group,
a n-butyl group,
an isobutyl group,
a s-butyl group, and
a t-butyl group.

Substituted Alkyl Group (Set of Specific Examples G3B):
a heptafluoropropyl group (including isomers),
a pentafluoroethyl group,
a 2,2,2-trifluoroethyl group, and
a trifluoromethyl group.

Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

Unsubstituted Alkenyl Group (Set of Specific Examples G4A):
a vinyl group,
an allyl group,
a 1-butenyl group,
a 2-butenyl group, and
a 3-butenyl group.

Substituted Alkenyl Group (Set of Specific Examples G4B):
a 1,3-butanedienyl group,
a 1-methylvinyl group,
a 1-methylallyl group,
a 1,1-dimethylallyl group,
a 2-methylallyl group, and
a 1,2-dimethylallyl group.

Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

Unsubstituted Alkynyl Group (Set of Specific Examples G5A):
an ethynyl group.

Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

Unsubstituted Cycloalkyl Group (Set of Specific Examples G6A):
a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

Substituted Cycloalkyl Group (Set of Specific Examples G6B):
a 4-methylcyclohexyl group.

Group Represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
Plural groups represented by G1 in —Si(G1)(G1)(G1) are the same as or different from each other.
Plural groups represented by G2 in —Si(G1)(G2)(G2) are the same as or different from each other.
Plural groups represented by G1 in —Si(G1)(G1)(G2) are the same as or different from each other.
Plural groups represented by G2 in —Si(G2)(G2)(G2) are the same as or different from each other.
Plural groups represented by G3 in —Si(G3)(G3)(G3) are the same as or different from each other.
Plural groups represented by G6 in —Si(G6)(G6)(G6) are the same as or different from each other.

Group Represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Group Represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Group Represented by —N(R₉₀₆)(R₉₀₇)

In the description herein, specific examples (set of specific examples G10) of the group represented by —N(R₉₀₆)(R₉₀₇) include:
—N(G1) (G1),
—N(G2)(G2),
—N(G1)(G2),
—N(G3)(G3), and
—N(G6)(G6).
   G1 represents the “substituted or unsubstituted aryl group” described in the set of specific examples 01,
   G2 represents the “substituted or unsubstituted heterocyclic group” described in the set of specific examples G2,
   G3 represents the “substituted or unsubstituted alkyl group” described in the set of specific examples G3, and
   G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

Halogen Atom

In the description herein, specific examples (set of specific examples 011) of the “halogen atom” include a fluorine atom, a chlorine atom, a bromine atom, and an iodine atom.

Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a ß-naphthylmethyl group, a 1-ß-naphthylethyl group, a 2-ß-naphthylethyl group, a 1-ß-naphthylisopropyl group, and a 2-ß-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

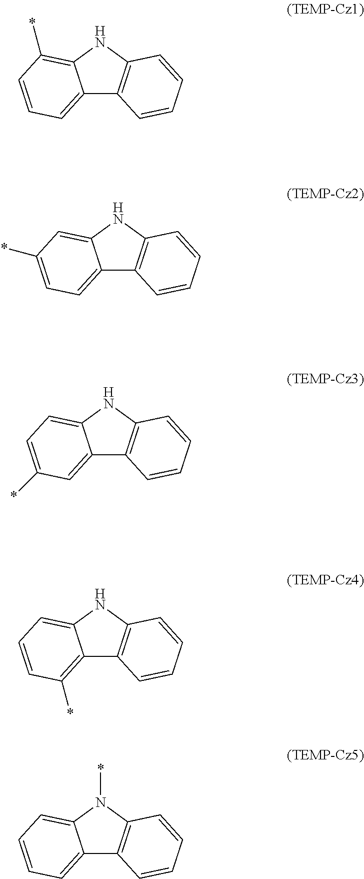 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

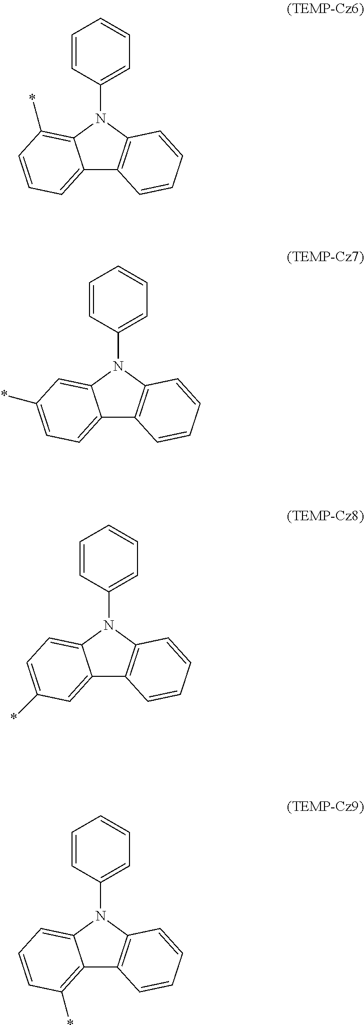 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

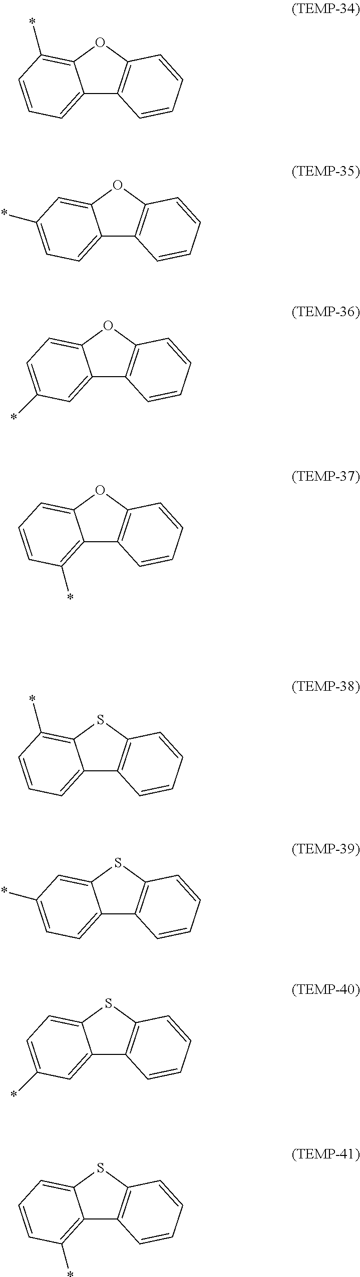

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl ring from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

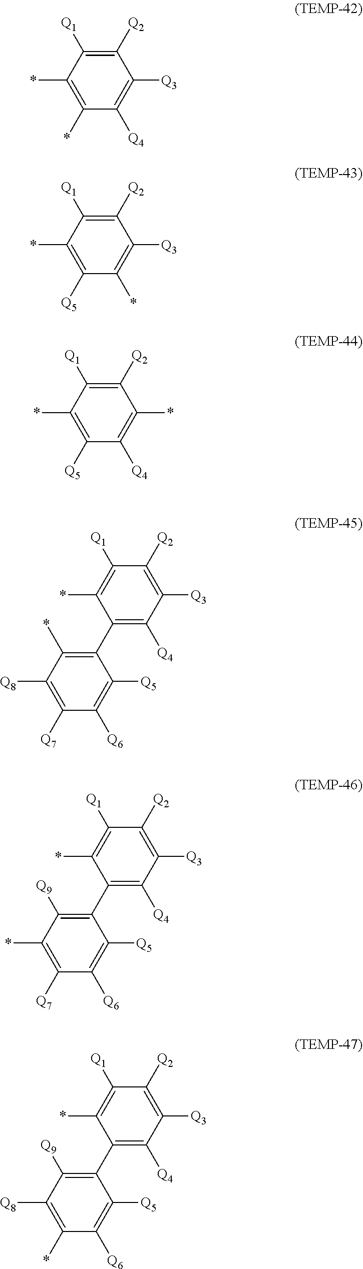

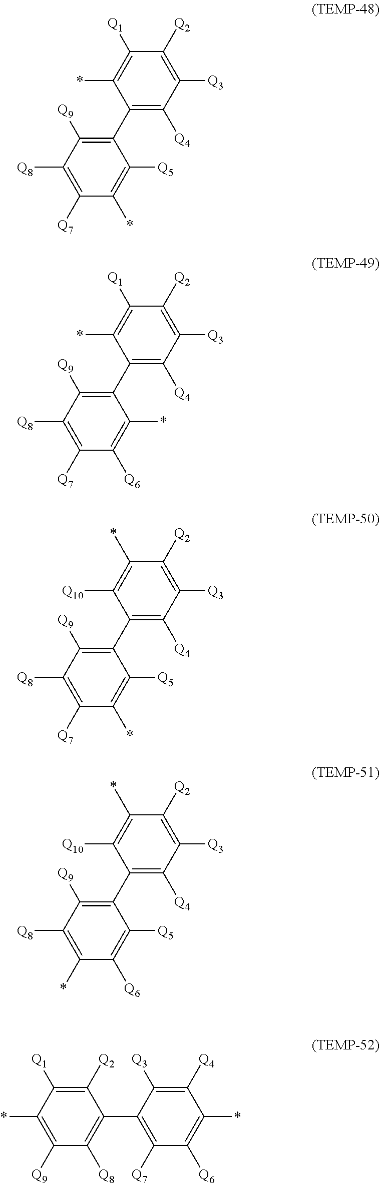

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

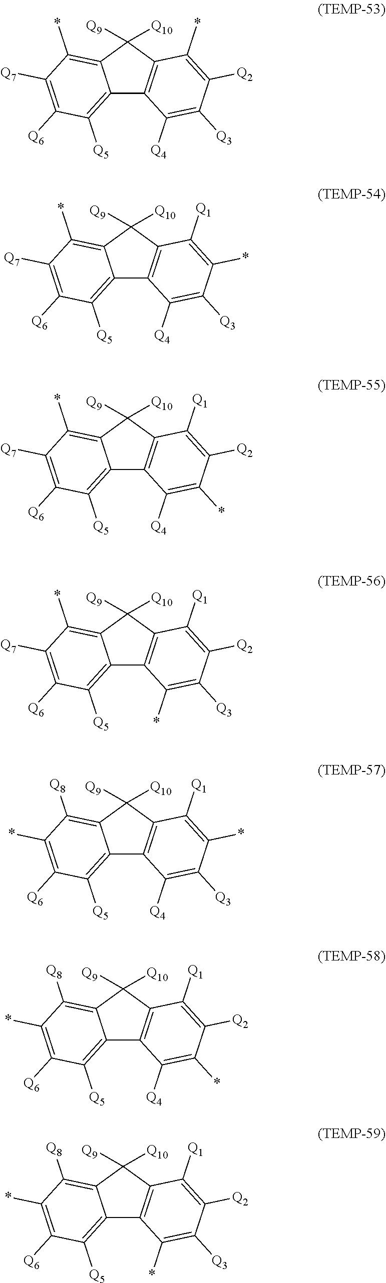

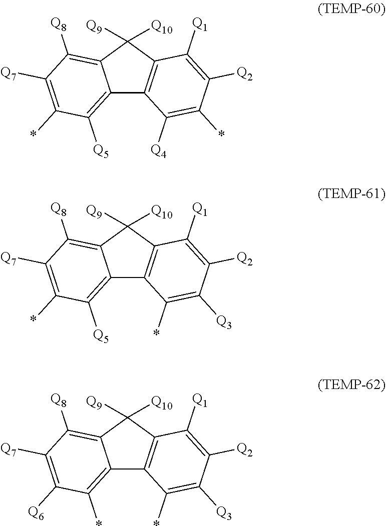

In the general formulae (TEMP-53) to (TEMP-62), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

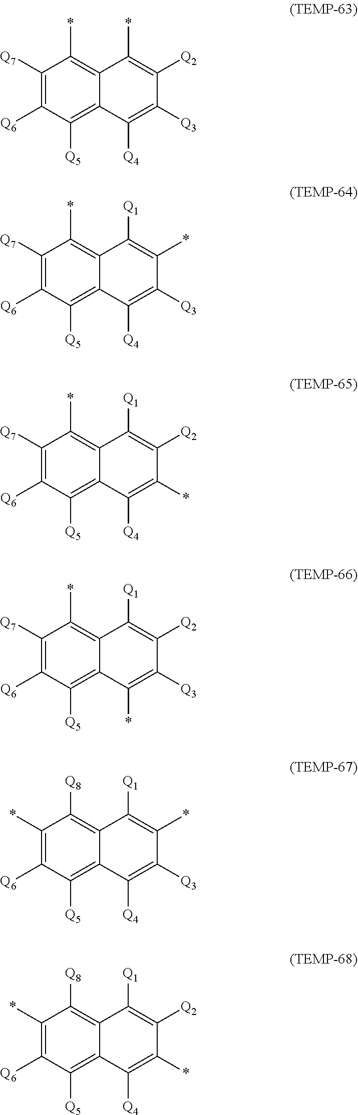

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

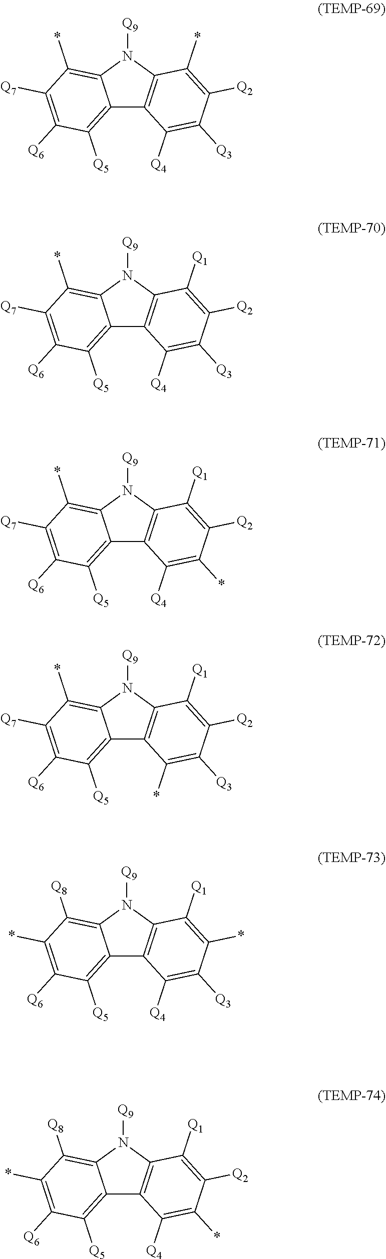

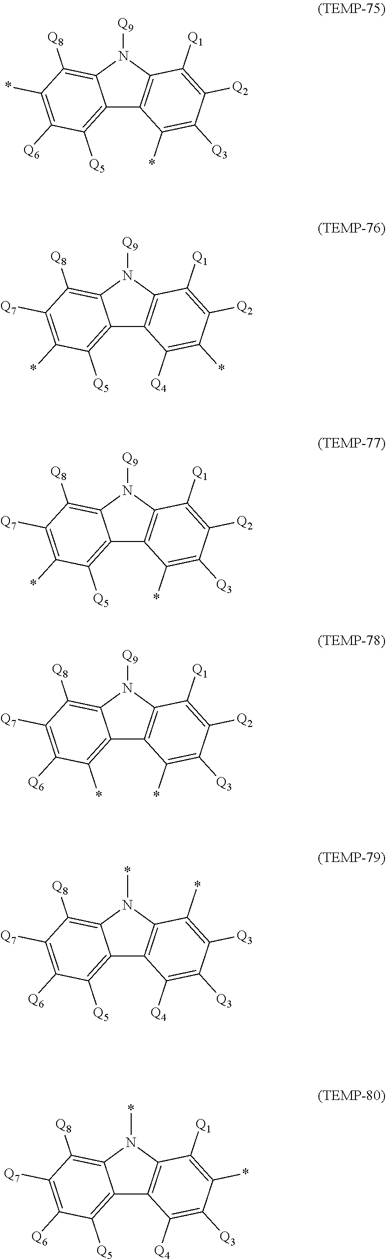

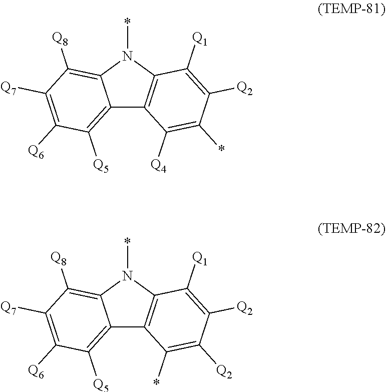

In the general formulae (TEMP-69) to (TEMP-82), Q₁ to Q₉ each independently represent a hydrogen atom or a substituent.

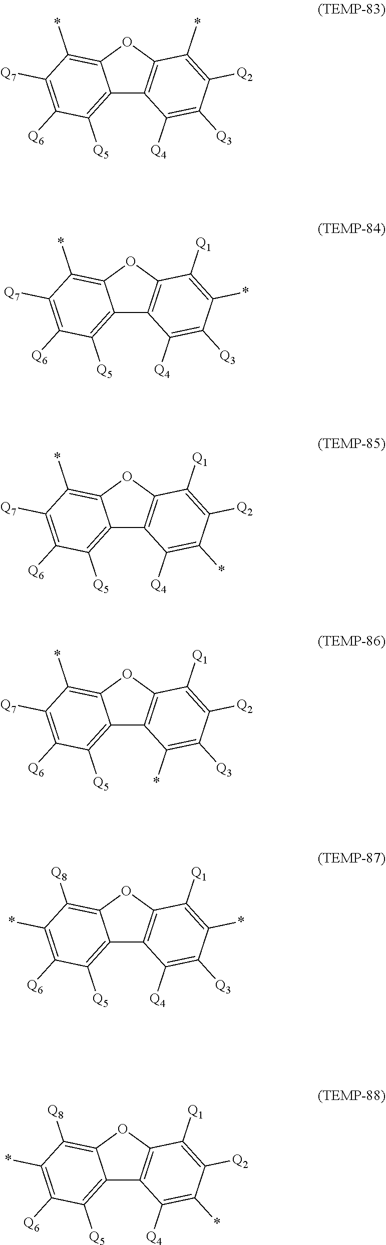

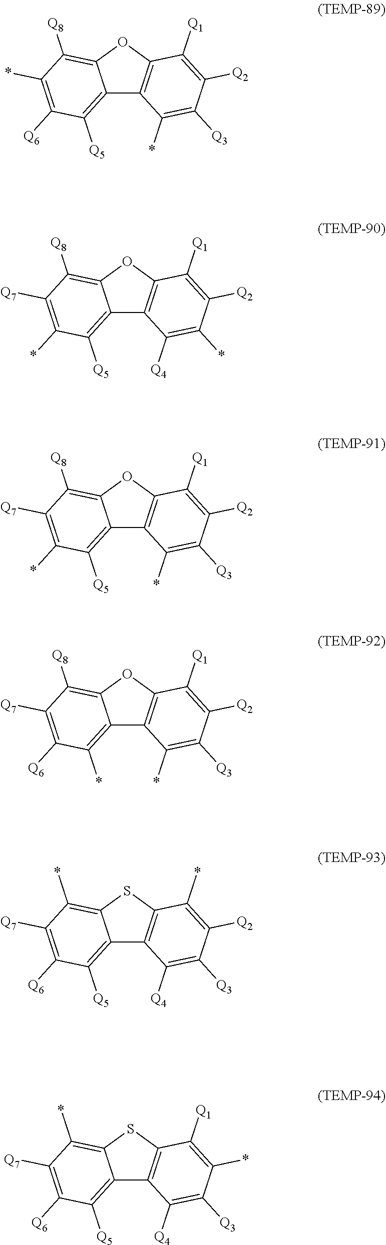

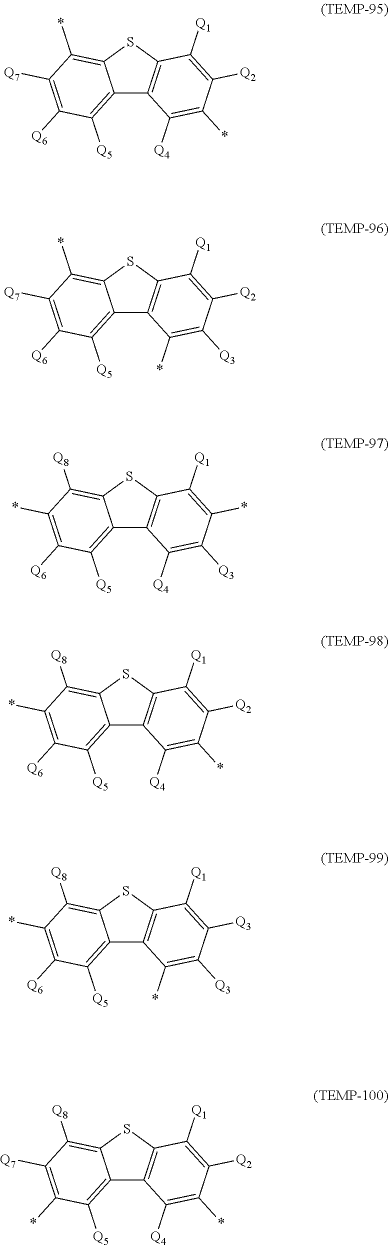

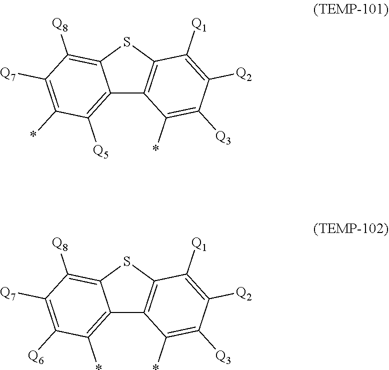

In the general formulae (TEMP-83) to (TEMP-102), Q₁ to Q₈ each independently represent a hydrogen atom or a substituent.

The above are the explanation of the “substituents in the description herein”.

Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

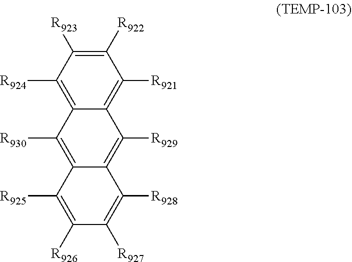

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

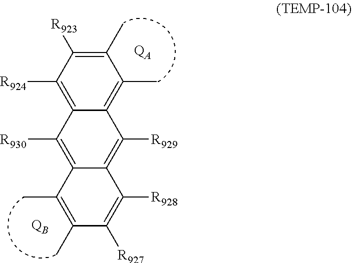

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

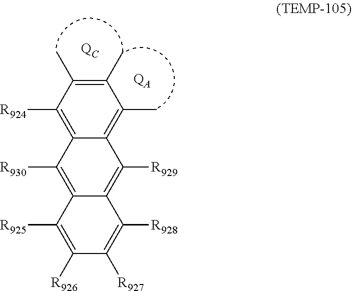

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O— (R₉₀₄),
—S—(R₉₀₅),
—N(R₉₀₆) (R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

Hereinafter, a compound of the present invention will be described.

The compound of the present invention is represented by the following formula (1). Hereinafter, the compound of the present invention, which is represented by the formula (1) and formulas included in the formula (1) which will be described below, may be simply referred to as an “invention compound”.

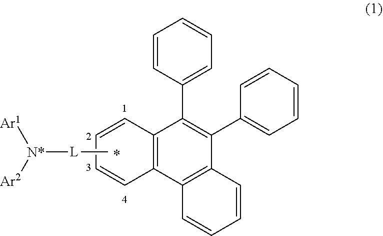

Hereinafter, descriptions will be made on symbols in the formula (1) and formulas included in the formula (1) which will be described below. The same symbols have the same meanings.
N* is a central nitrogen atom.
* is bonded to the carbon atom 1, 2, 3 or 4, preferably the carbon atom 1 or 2.
L is a single bond, an unsubstituted phenylene group, or an unsubstituted biphenylene group, preferably a single bond or an unsubstituted phenylene group (an o-phenylene group, a m-phenylene group, or a p-phenylene group). In an aspect of the present invention, L is a single bond, and in another preferred aspect, L is an unsubstituted phenylene group.
Ar¹ is a group represented by the following formula (1a), (1b), (1c), (1d), (1e), or (1f).

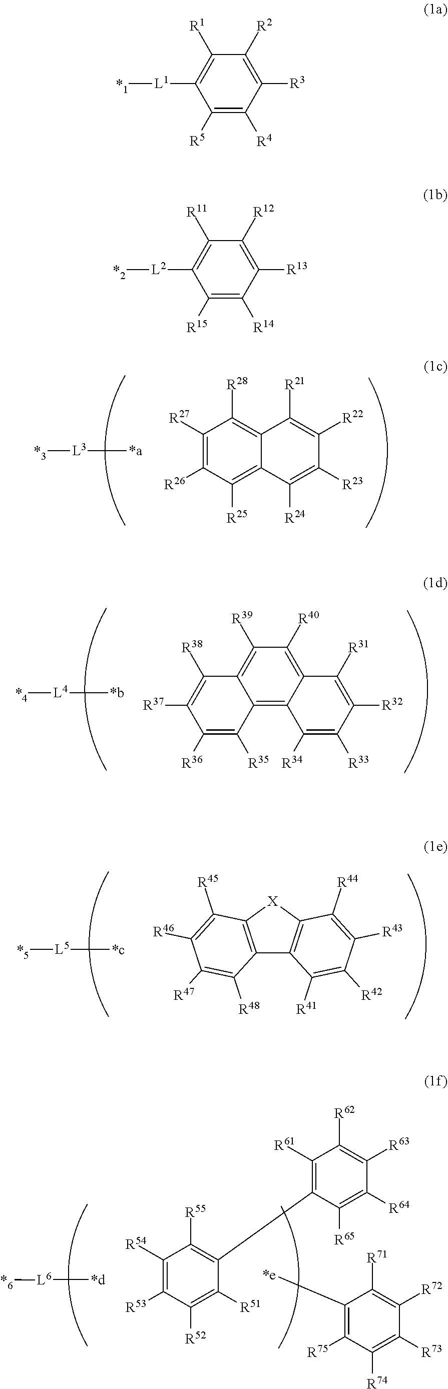

Ar₂ is a group represented by the following formula (1b), (1c), (1d), (1e), (1f), or (1g).

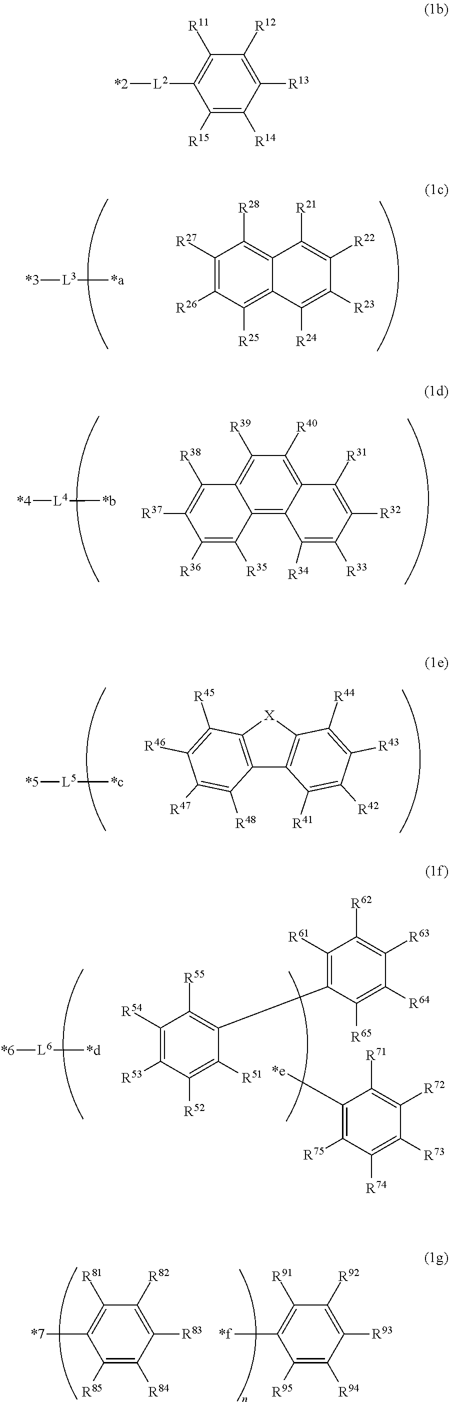

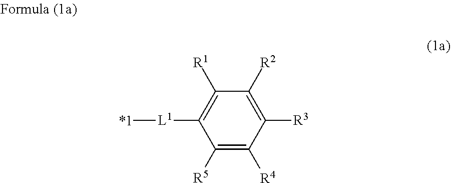

in which
each of R¹ to R⁵ is independently a hydrogen atom or a substituted or unsubstituted alkyl group having 1 to 6 carbon atoms;
adjacent two selected from R¹ to R⁵ are not bonded to each other, and thus do not form a ring;
L¹ is a single bond or an unsubstituted phenylene group; and
*1 is bonded to the central nitrogen atom N*.

The unsubstituted alkyl group having 1 to 6 carbon atoms is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, further preferably a methyl group or a t-butyl group.

All of R¹ to R⁵ may be hydrogen atoms.

In an aspect of the present invention, L¹ is a single bond, and in another aspect, L¹ is an unsubstituted phenylene group (an o-phenylene group, a m-phenylene group, or a p-phenylene group).

Therefore, the formula (1a) includes a group represented by the following formula (1a′) or (1a″).

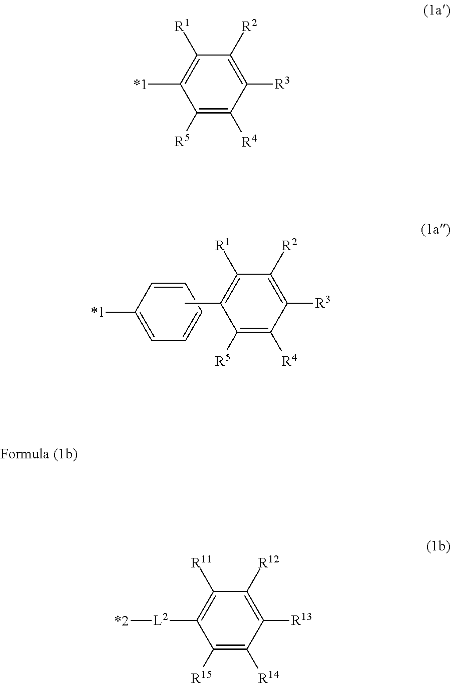

in which, each of R¹¹ to R¹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R¹¹ to R¹⁵ are not bonded to each other, and thus do not form a ring;
L² is an unsubstituted biphenylene group; and
*2 is bonded to the central nitrogen atom N*.

Details of the unsubstituted alkyl group having 1 to 6 carbon atoms are the same as those described for R¹ to R⁵ in the formula (1a).

The unsubstituted aryl group having 6 to 12 ring carbon atoms is preferably a phenyl group, a biphenyl group, or a naphthyl group, more preferably a phenyl group or a naphthyl group, further preferably a phenyl group.

The biphenyl group includes an o-biphenyl group, a m-biphenyl group and a p-biphenyl group, and a m-biphenyl group and a p-biphenyl group are preferred, and a p-biphenyl group is more preferred.

The naphthyl group includes a 1-naphthyl group and a 2-naphthyl group, and a 1-naphthyl group is preferred.

L² is preferably a 4,4′-biphenylene group or a 3,4′-biphenylene group, more preferably a 4,4′-biphenylene group.

All of R¹¹ to R¹⁵ may be hydrogen atoms.

The formula (1b) includes a group represented by the following formula (1b′).

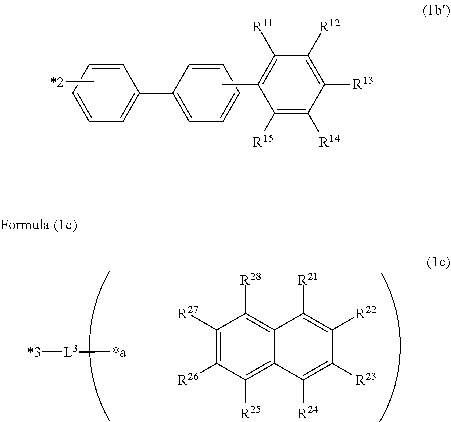

in which, each of R²¹ to R²⁸ is independently a hydrogen atom, a substituted or unsubstituted alkyl group having 1 to 6 carbon atoms, or a substituted or unsubstituted aryl group having 6 to 12 ring carbon atoms;
provided that one selected from R²¹ to R²⁸ is a single bond that binds to *a, and adjacent two selected from R²¹ to R²⁸ except for the single bond are not bonded to each other, and thus do not form a ring structure;
L³ is a single bond or an unsubstituted arylene group having 6 to 12 ring carbon atoms; and
*3 is bonded to the central nitrogen atom N*.

Details of the unsubstituted alkyl group having 1 to 6 carbon atoms are the same as those described for R¹ to R⁵ in the formula (1a), and details of the unsubstituted aryl group having 6 to 12 ring carbon atoms are the same as those described for R¹¹ to R¹⁵ in the formula (1b).

The unsubstituted arylene group having 6 to 12 ring carbon atoms is preferably a phenylene group, a biphenylene group or a naphthylene group, more preferably a phenylene group or a biphenylene group, further preferably a phenylene group.

The phenylene group is preferably a m-phenylene group or a p-phenylene group, more preferably a p-phenylene group.

The biphenylene group is preferably a 4,4′-biphenylene group or a 3,4′-biphenylene group, more preferably a 4,4′-biphenylene group.

The naphthylene group is preferably a 1,4-naphthylene group or a 2,6-naphthylene group, more preferably a 1,4-naphthylene group.

In an aspect of the present invention, R²¹ is a single bond bonded to *a, and in another aspect, R²² is a single bond bonded to *a.

All of R²¹ to R²⁸ except for the single bond bonded to *a may be hydrogen atoms.

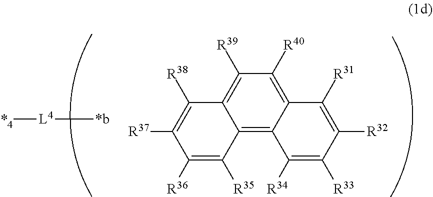

in which, each of R³¹ to R⁴⁰ is independently a hydrogen atom, a substituted or unsubstituted alkyl group having 1 to 6 carbon atoms, or a substituted or unsubstituted aryl group having 6 to 12 ring carbon atoms;
provided that one selected from R³¹ to R⁴⁰ is a single bond bonded to *b, and adjacent two selected from R³¹ to R⁴⁰ except for the single bond are not bonded to each other, and thus do not form a ring structure;
L⁴ is a single bond or an unsubstituted arylene group having 6 to 12 ring carbon atoms; and
*4 is bonded to the central nitrogen atom N*.

Details of the unsubstituted alkyl group having 1 to 6 carbon atoms are the same as those described for R¹ to R⁵ in the formula (1a), details of the unsubstituted aryl group having 6 to 12 ring carbon atoms are the same as those described for R¹¹ to R¹⁵ in the formula (1b), and details of the unsubstituted arylene group having 6 to 12 ring carbon atoms are the same as those described for L³ in the formula (1c).
L⁴ is preferably a single bond or an unsubstituted phenylene group. L⁴ is a single bond in an aspect of the present invention, and is an unsubstituted phenylene group in another aspect.

One selected from R³⁷, R³⁸ and R³⁹ is preferably a single bond bonded to *b. R³⁷ in an aspect of the present invention, R³⁸ in another aspect, and R³⁹ in yet another aspect are single bonds bonded to *b.

All of R³¹ to R⁴⁹ except for the single bond bonded to *b may be hydrogen atoms.

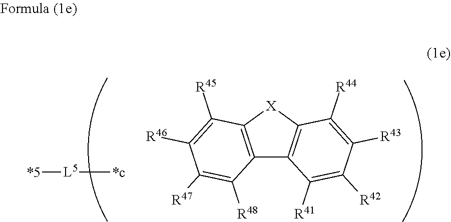

in which, X is an oxygen atom, a sulfur atom, NRa or CRbRc;
Ra is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
Rb is a substituted or unsubstituted aryl group having 6 to 12 ring carbon atoms;
Rc is a substituted or unsubstituted alkyl group having 1 to 6 carbon atoms or a substituted or unsubstituted aryl group having 6 to 12 ring carbon atoms;
Rb and Rc may be bonded to each other to form a spiro ring structure;
each of R⁴¹ to R⁴⁸ is independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
provided that one selected from the R⁴¹ to R⁴⁸ and Ra is a single bond bonded to *c;
adjacent two selected from R⁴¹ to R⁴⁸ except for the single bond may be bonded to each other to form one or more substituted or unsubstituted benzene rings. Meanwhile, when X is CRbRc, the adjacent two do not form a ring;
L⁵ is a single bond or an unsubstituted arylene group having 6 to 12 ring carbon atoms; and
*5 is bonded to the central nitrogen atom N*.

In an aspect of the present invention, the formula (1e) is represented by the following formula,

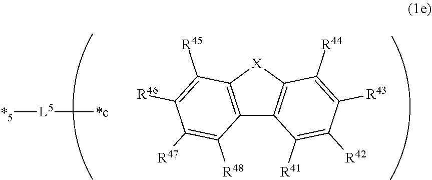

in another aspect, the formula (1e) is represented by the following formula,

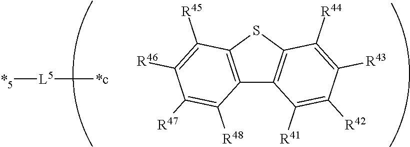

in another aspect, the formula (1e) is represented by the following formula,

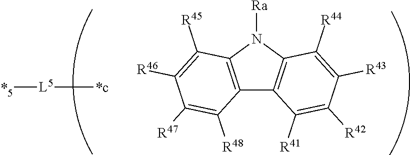

in another aspect, the formula (1e) is represented by the following formula.

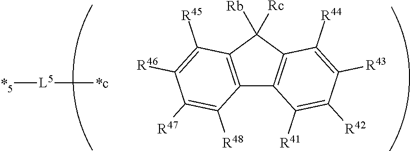

For each unsubstituted alkyl group having 1 to 6 carbon atoms, which is described above, details are the same as those described for R¹ to R⁵ in the formula (1a), for each unsubstituted aryl group having 6 to 12 ring carbon atoms, which is described above, details are the same as those described for R¹¹ to R¹⁵ in the formula (1b), and details of the unsubstituted arylene group having 6 to 12 ring carbon atoms are the same as those described for L³ in the formula (1c).

The unsubstituted aromatic heterocyclic group having 5 to 13 ring atoms is preferably a pyrrolyl group, a furyl group, a thienyl group, a pyridyl group, a pyrimidinyl group, a triazinyl group, a quinolyl group, an isoquinolyl group, a quinazolinyl group, a benzoimidazolyl group, a benzofuranyl group, a benzothiophenyl group (benzothienyl group), a carbazolyl group, a dibenzofuranyl group, or a dibenzothiophenyl group (dibenzothienyl group).

Ra is preferably an unsubstituted phenyl group or an unsubstituted naphthyl group (1-naphthyl group or 2-naphthyl group).

It is desirable that both Rb and Rc are phenyl groups, or Rb is a phenyl group, and Rc is a methyl group.

Rb and Rc may be bonded to each other to form the following Spiro ring compound.

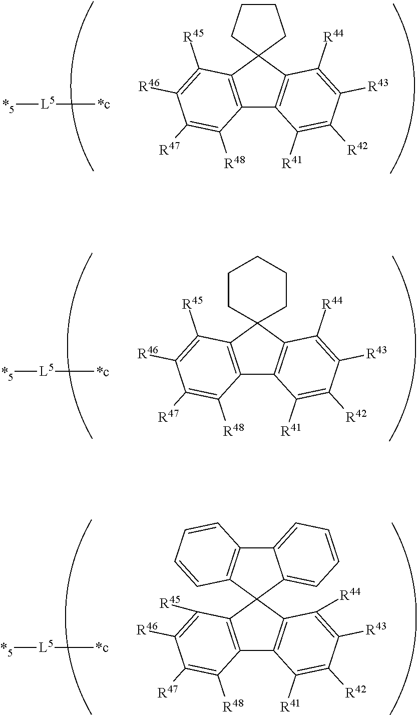

When X is an oxygen atom or a sulfur atom, one selected from R⁴⁵ to R⁴⁸ is preferably a single bond bonded to *c.

When X is NRa, one selected from R⁴⁵ to R⁴⁸ is preferably a single bond bonded to *c.

When X is CRbRc, one selected from R⁴⁵ to R⁴⁸ is preferably a single bond bonded to *c.

L⁵ is preferably a single bond or an unsubstituted phenylene group. L⁵ is a single bond in an aspect of the present invention, and is an unsubstituted phenylene group in another aspect.

All of R⁴¹ to R⁴⁸ except for the single bond bonded to *c may be hydrogen atoms.

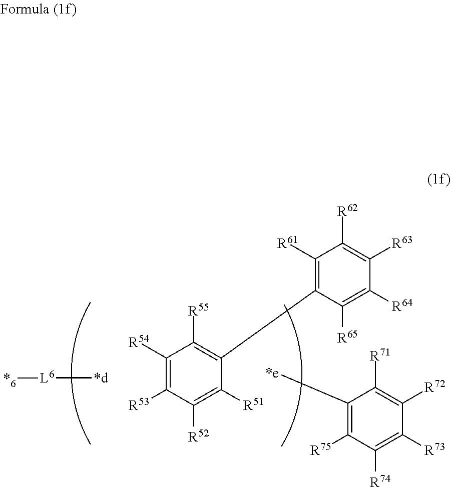

in which,
each of R⁵¹ to R⁵⁵ is independently a hydrogen atom, an unsubstituted alkyl group having 1 to 6 carbon atoms, or an unsubstituted phenyl group;
provided that one selected from R⁵¹ to R⁵⁵ is a single bond bonded to *d, and another selected from R⁵¹ to R⁵⁵ is a single bond bonded to *e;
except for the single bond bonded to *d and the single bond bonded to *e, adjacent two selected from R⁵¹ to R⁵⁵ are not bonded to each other, and thus do not form a ring structure;
each of R⁶¹ to R⁶⁵ and R⁷¹ to R⁷⁵ is independently a hydrogen atom or an unsubstituted alkyl group having 1 to 6 carbon atoms;
adjacent two selected from R⁶¹ to R⁶⁵ and R⁷¹ to R⁷⁵ may be bonded to each other to form one or more substituted or unsubstituted benzene rings;
L⁶ is a single bond or an unsubstituted arylene group having 6 to 12 ring carbon atoms; and
*6 is bonded to the central nitrogen atom N*.

For each unsubstituted alkyl group having 1 to 6 carbon atoms, which is described above, details are the same as those described for R¹ to R⁵ in the formula (1a), and details of the unsubstituted arylene group having 6 to 12 ring carbon atoms are the same as those described for L³ in the formula (1c).

In an aspect of the present invention, one or more substituted or unsubstituted benzene rings are formed through mutual bonding in adjacent two selected from R⁶¹ to R⁶⁵, in which one or more sets of adjacent two is (are) selected from R⁶¹ and R⁶², R⁶² and R⁶³, R⁶³ and R⁶⁴, and R⁶⁴ and R⁶⁵. In another aspect of the present invention, adjacent two selected from R⁶¹ to R⁶⁵ are not bonded to each other and thus do not form a ring structure.

In an aspect of the present invention, one or more substituted or unsubstituted benzene rings are formed through mutual bonding in adjacent two selected from R⁷¹ to R⁷⁵, in which one or more sets of adjacent two is (are) selected from R⁷¹ and R⁷², R⁷² and R⁷³, R⁷³ and R⁷⁴, and R⁷⁴ and R⁷⁵. In another aspect of the present invention, adjacent two selected from R⁷¹ to R⁷⁵ are not bonded to each other and thus do not form a ring structure.

L⁶ is preferably a single bond or an unsubstituted phenylene group. L⁶ is a single bond in an aspect of the present invention, and is an unsubstituted phenylene group in another aspect.

Except for the single bond bonded to *d and the single bond bonded to *e, all of R⁵¹ to R⁵⁵ may be hydrogen atoms, all of R⁶¹ to R⁶⁵ may be hydrogen atoms, and all of R⁷¹ to R⁷⁵ may be hydrogen atoms.

The formula (1f) includes groups represented by the following formulas (1fa) to (1fe).

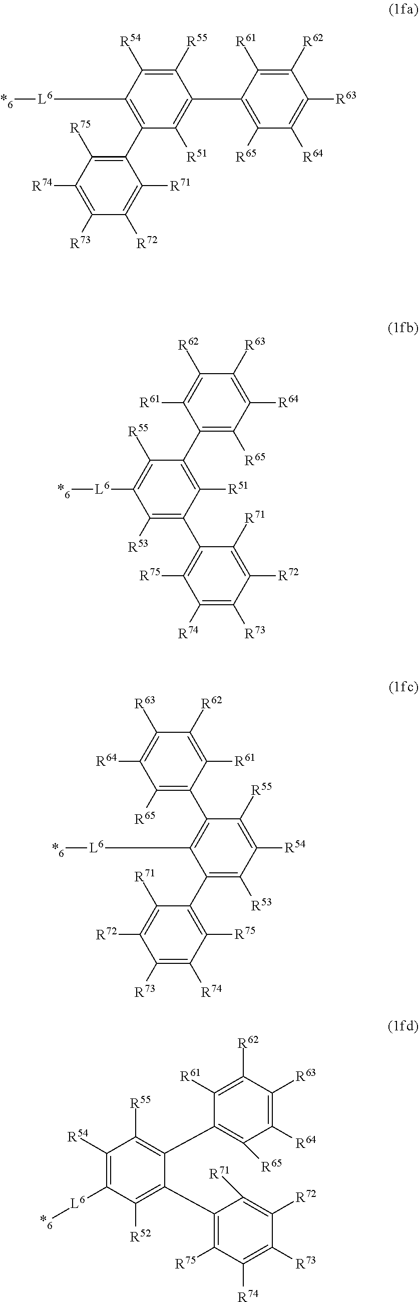

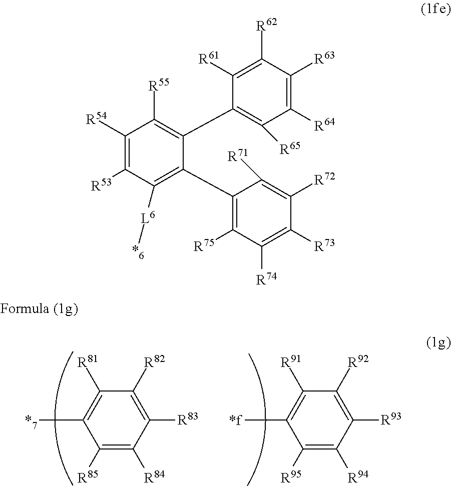

in which, each of R⁸¹ to R⁸⁵ is independently a hydrogen atom or a substituted or unsubstituted alkyl group having 1 to 6 carbon atoms;
provided that one selected from R⁸¹, R⁸², R⁸⁴, and R⁸⁵ is a single bond bonded to *f;
adjacent two selected from R⁸³ and R⁸¹, R⁸², R⁸⁴, and R⁸⁵ except for the single bond bonded to *f are not bonded to each other, and thus do not form a ring structure;
each of R⁹¹ to R⁹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R⁹¹ to R⁹⁵ are not bonded to each other, and thus do not form a ring structure;
n is 0 or 1, and then when n is 0, the formula (1g) indicates the following formula (1g′); and

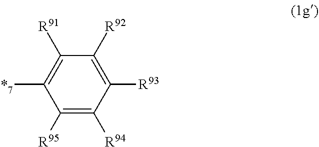

*7 in the formulas (1g) and (1g′) is bonded to the central nitrogen atom.

For each unsubstituted alkyl group having 1 to 6 carbon atoms, which is described above, details are the same as those described for R¹ to R⁵ in the formula (1a), and for each unsubstituted aryl group having 6 to 12 ring carbon atoms, which is described above, details are the same as those described for R¹¹ to R¹⁵ in the formula (1b).
R⁸³ and all of R⁸¹, R⁸², R⁸⁴, and R⁸⁵ except for the single bond bonded to *f may be hydrogen atoms, and all of R⁹¹ to R⁹⁵ may be hydrogen atoms.

The formula (1g) includes a group represented by the following formula (1g′), (1g″), or (1g′″).

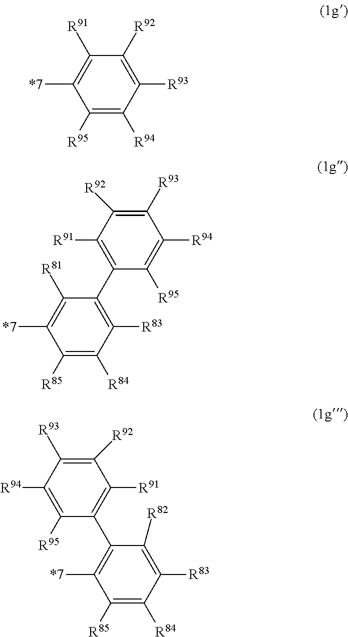

As described above, Ar¹ is selected from groups represented by the formulas (1a), (1b), (1c), (1d), (1e) and (1f), and Are is selected from groups represented by the formulas (1b), (1c), (1d), (1e), (1f) and (1g). Therefore, the invention compound includes the following compounds.

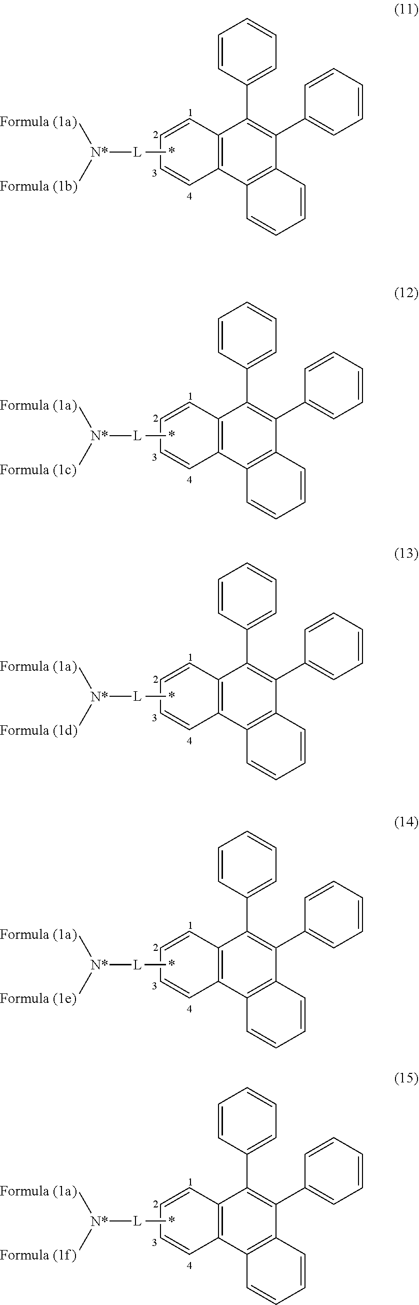

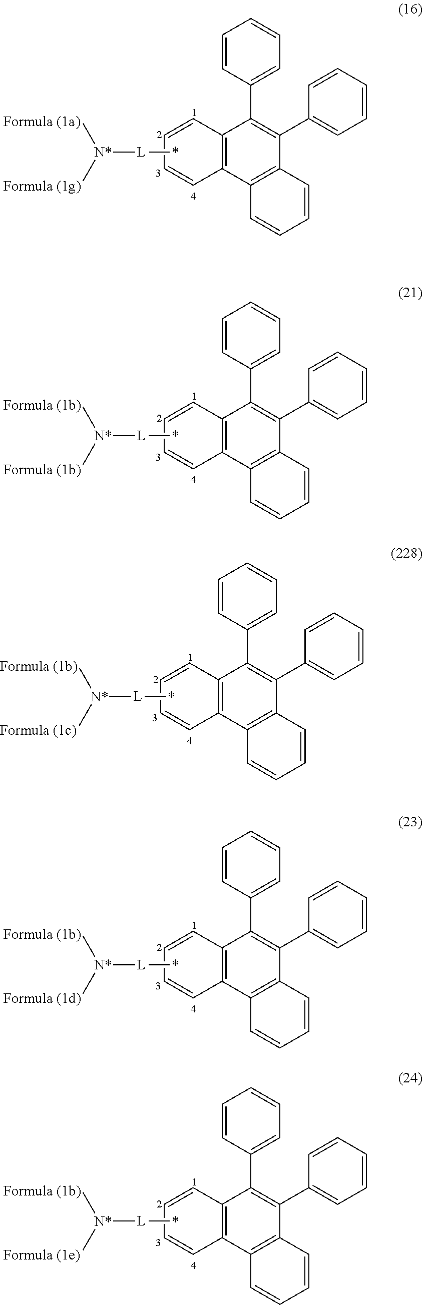

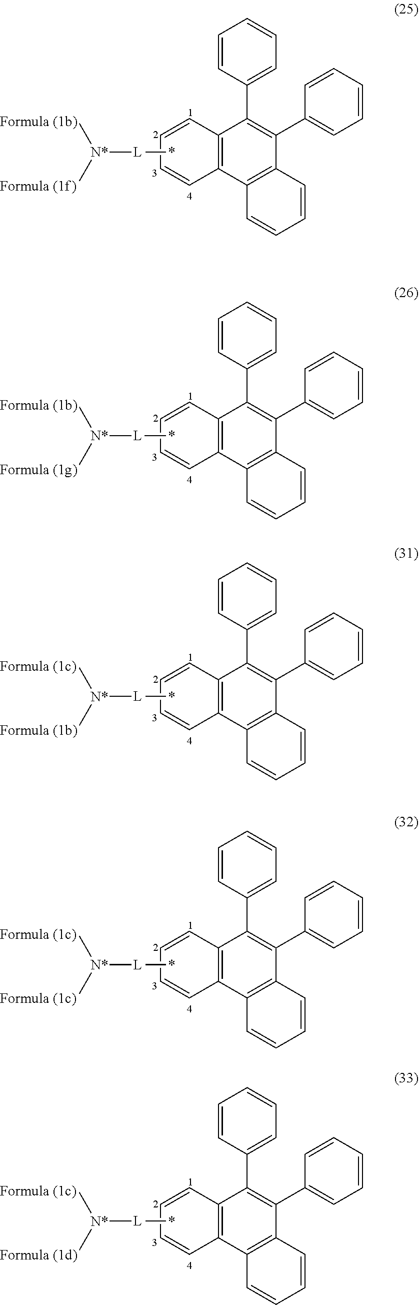

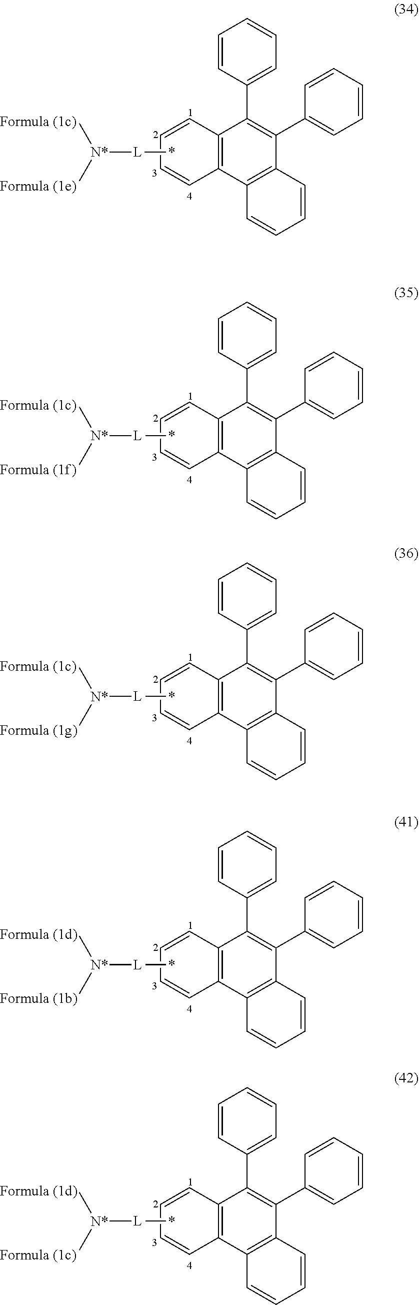

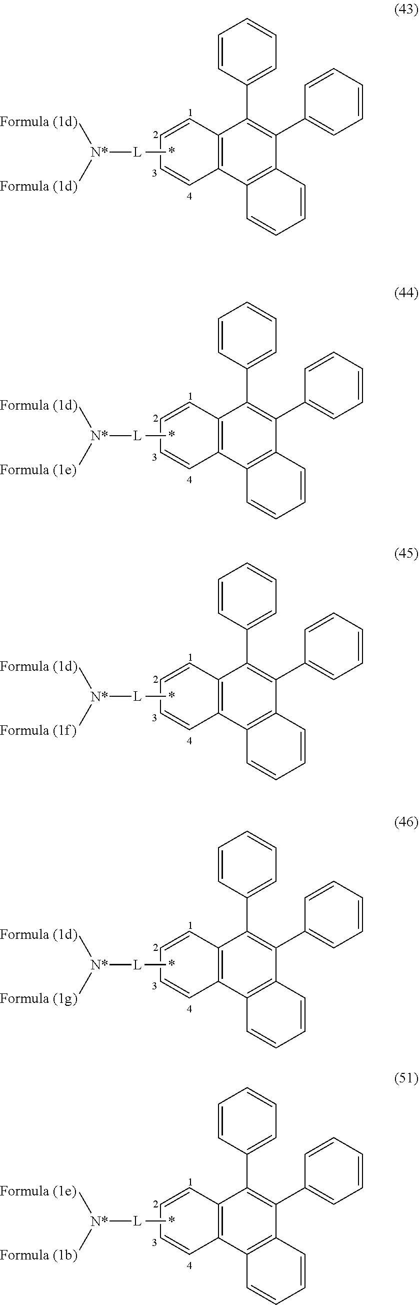

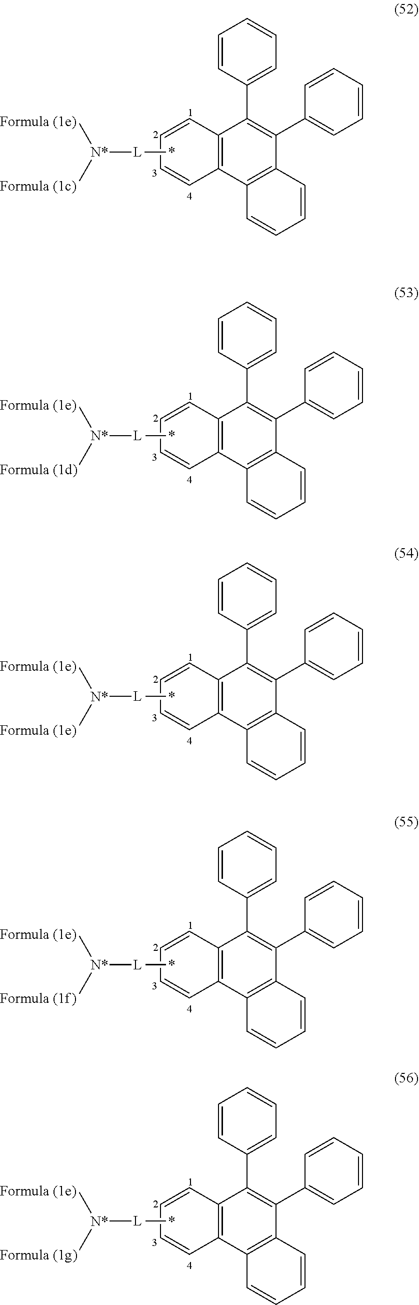

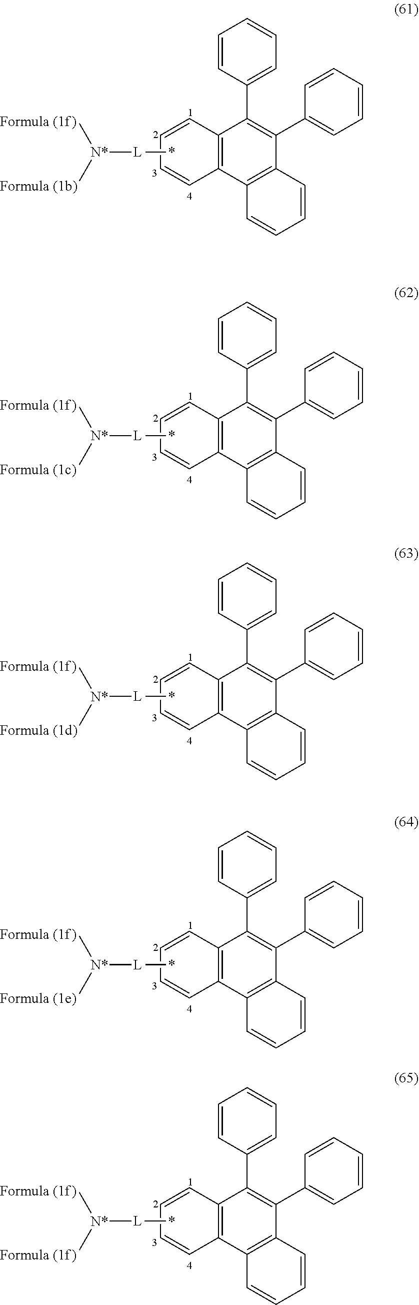

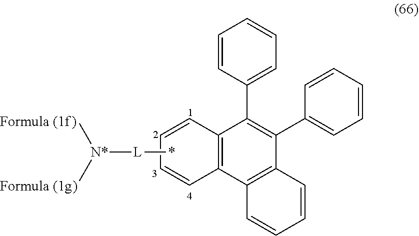

Among the above invention compounds,
preferred is a compound in which Ar¹ is a group represented by the formula (1a), (1c), (1d), or (1f), and Ar² is a group represented by the formula (1b), (1c), (1d), (1e), (1f), or (1g),
more preferred is a compound in which Ar¹ is a group represented by the formula (1a), (1c), (1d), or (1f), and Ar² is a group represented by (1c), (1d), (1e), (1f), or (1g), and
further preferred is a compound in which Ar¹ is a group represented by the formula (1a), (1c), (1d), or (1f), and Ar² is a group represented by (1c), (1e), (1f), or (1g).

As described above, the “hydrogen atom” used in this specification includes a protium atom, a deuterium atom, and a tritium atom. Therefore, the invention compound may contain a naturally derived deuterium atom.

Further, by using a deuterated compound for a part or all of raw material compounds, deuterium atoms may be intentionally introduced into the invention compound A. Therefore, in an aspect of the present invention, the invention compound contains at least one deuterium atom. That is, the invention compound is a compound represented by the formula (1), which may be a compound in which at least one of hydrogen atoms contained in the corresponding compound is a deuterium atom.

At least one hydrogen atom selected from the following hydrogen atoms may be a deuterium atom. Hereinafter, “substituted or unsubstituted”, the number of carbon atoms, and the number of atoms are omitted.

A hydrogen atom included in a diphenylphenanthryl group of the formula (1);
a hydrogen atom included in a phenylene group or a biphenylene group when L of the formula (1) is the corresponding phenylene group or the biphenylene group;
a hydrogen atom represented by any of R¹ to R⁵ in the formula (1a);
a hydrogen atom included in an alkyl group when any of R¹ to R⁵ in the formula (1a) is the corresponding alkyl group;
a hydrogen atom included in a phenylene group when L¹ of the formula (1a) is the corresponding phenylene group;
a hydrogen atom represented by any of R¹¹ to R¹⁵ in the formula (1b);
a hydrogen atom included in an alkyl group or an aryl group when any of R¹¹ to R¹⁵ in the formula (1b) is the corresponding alkyl group or the aryl group;
a hydrogen atom included in L² (biphenylene group) of the formula (1b);
a hydrogen atom represented by any of R²¹ to R²⁸ of the formula (1c);
a hydrogen atom included in an alkyl group or an aryl group when any of R²¹ to R²⁸ of the formula (1c) is the corresponding alkyl group or the aryl group;
a hydrogen atom included in an arylene group when L³ of the formula (1c) is the corresponding arylene group;
a hydrogen atom represented by any of R³¹ to R⁴⁰ in the formula (1d);
a hydrogen atom included in an alkyl group or an aryl group when any of R³¹ to R⁴⁰ in the formula (1d) is the corresponding alkyl group or the aryl group;
a hydrogen atom included in an arylene group when L⁴ of the formula (1d) is the corresponding arylene group;
a hydrogen atom represented by any of R⁴¹ to R⁴⁸ and Ra in the formula (1e);
a hydrogen atom included in an alkyl group, an aryl group or an aromatic heterocyclic group when any of R⁴¹ to R⁴⁸ and Ra in the formula (1e) is the corresponding alkyl group, the aryl group or the aromatic heterocyclic group;
a hydrogen atom included in Rb (an aryl group) of the formula (1e);
a hydrogen atom included in Rc (an alkyl group or an aryl group) of the formula (1e);
a hydrogen atom included in an arylene group when L⁵ of the formula (1e) is the corresponding arylene group;
a hydrogen atom represented by any of R⁵¹ to R⁵⁵, R⁸¹ to R⁶⁵, and R⁷¹ to R⁷⁵ in the formula (1f);
a hydrogen atom included in an alkyl group or a phenyl group when any of R⁵¹ to R⁵⁵ in the formula (1f) is the corresponding alkyl group or the phenyl group;
a hydrogen atom included in an alkyl group when any of R⁶¹ to R⁶⁵ and R⁷¹ to R⁷⁵ in the formula (1f) is the corresponding alkyl group;
a hydrogen atom included in an arylene group when L⁶ of the formula (1f) is the corresponding arylene group;
a hydrogen atom represented by any of R⁸¹ to R⁸⁵ and R⁹¹ to R⁹⁵ in the formula (1f);
a hydrogen atom included in an alkyl group when R⁸¹ to R⁸⁵ of the formula (1g) are the corresponding alkyl groups; and
a hydrogen atom included in an alkyl group or an aryl group when any of R⁹¹ to R⁹⁵ in the formula (1g) is the corresponding alkyl group or the aryl group.

The deuteration rate of the invention compound depends on the deuteration rates of raw material compounds to be used. Even if a raw material with a predetermined deuteration rate is used, a certain proportion of naturally derived protium isotopes may be contained. Therefore, in the aspect of the deuteration rate of the invention compound as illustrated below, in addition to the rate obtained by simply counting the number of deuterium atoms represented by the formula, the rate on which a trace amount of naturally derived isotopes are reflected is included.

The deuteration rate of the invention compound is preferably 1% or more, more preferably 3% or more, further preferably 5% or more, still further preferably 10% or more, still further preferably 50% or more.

The invention compound may be a mixture containing a deuterated compound and a non-deuterated compound, or a mixture of two or more compounds having different deuteration rates. The deuteration rate of such a mixture is preferably 1% or more, more preferably 3% or more, further preferably 5% or more, still further preferably 10% or more, still further preferably 50% or more, and is less than 100%.

Further, the ratio of the number of deuterium atoms to the total number of hydrogen atoms in the invention compound is preferably 1% or more, more preferably 3% or more, further preferably 5% or more, still further preferably 10% or more, and is 100% or less.

When the “substituted or unsubstituted XX group” included in the definition of each of the above described formulas is a substituted XX group, details of the corresponding substituent are the same as those described for “the substituent in the case of “substituted or unsubstituted””. Preferred is an alkyl group having 1 to 6 carbon atoms, an aryl group having 6 to 12 ring carbon atoms, or an aromatic heterocyclic group having 5 to 13 ring atoms, and more preferred is an alkyl group having 1 to 6 carbon atoms or an aryl group having 6 to 12 ring carbon atoms. Details of each group are the same as described above.

The invention compound can be easily produced by those skilled in the art with reference to the following synthesis examples and known synthesis methods.

Hereinafter, specific examples of the invention compound will be illustrated, but are not limited to the following exemplary compounds.

In the following specific examples, D represents a deuterium atom.

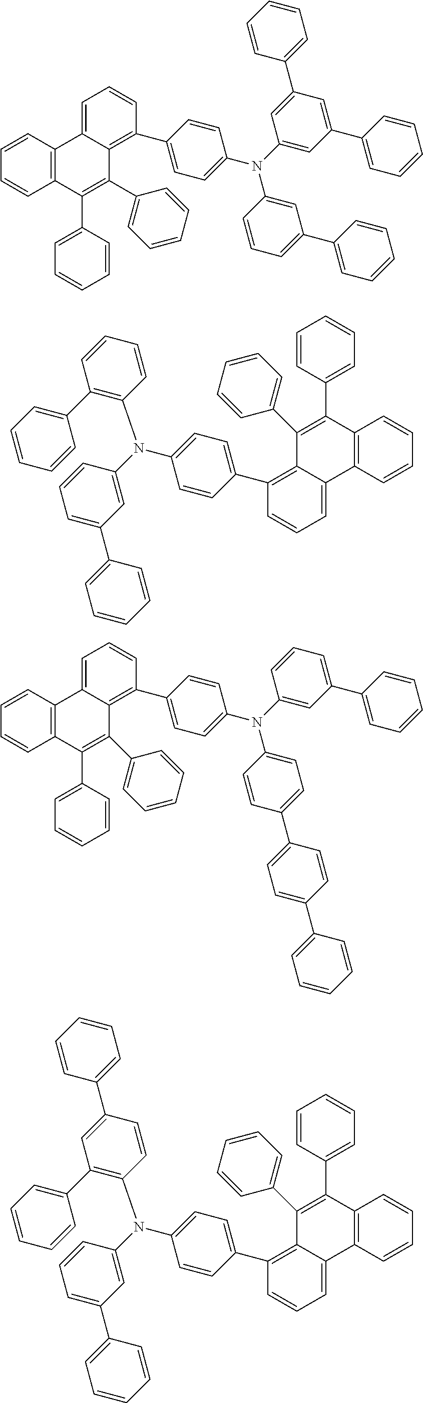

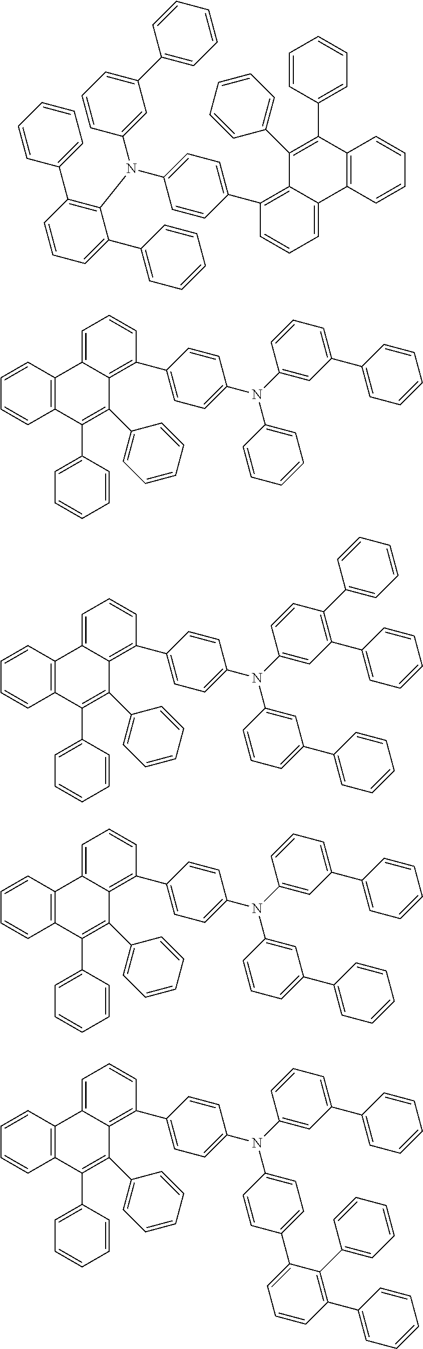

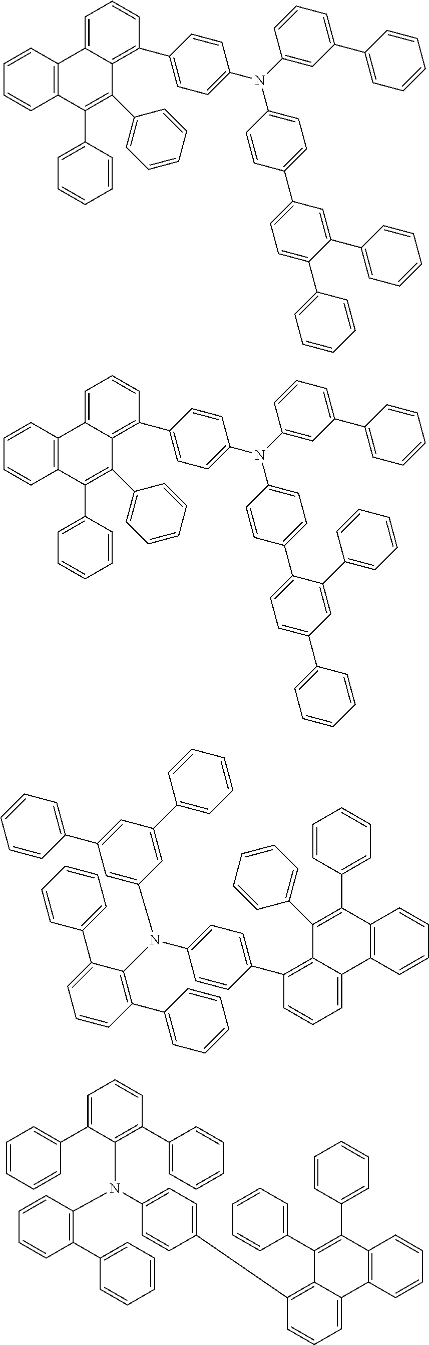

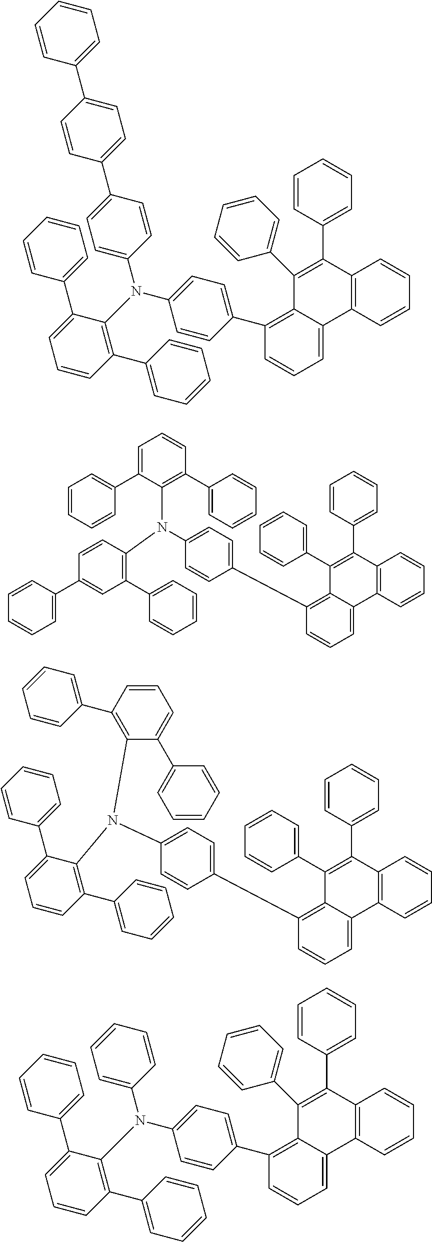

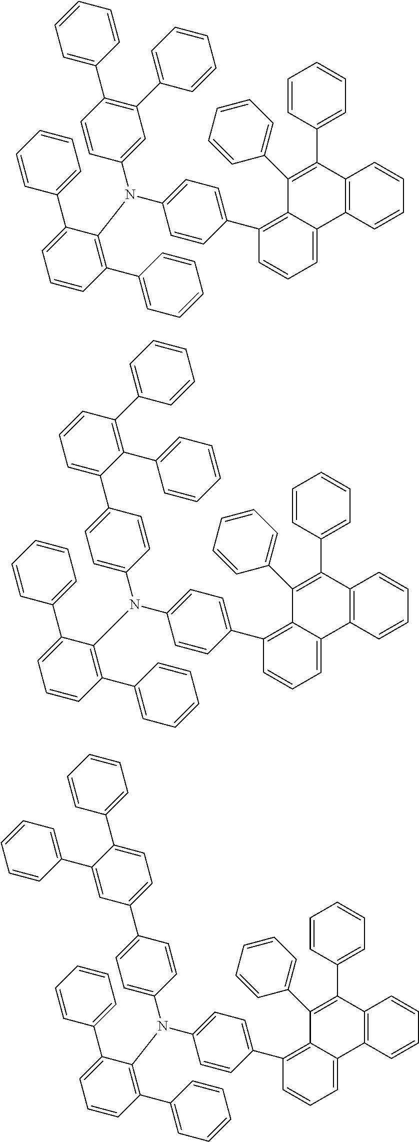

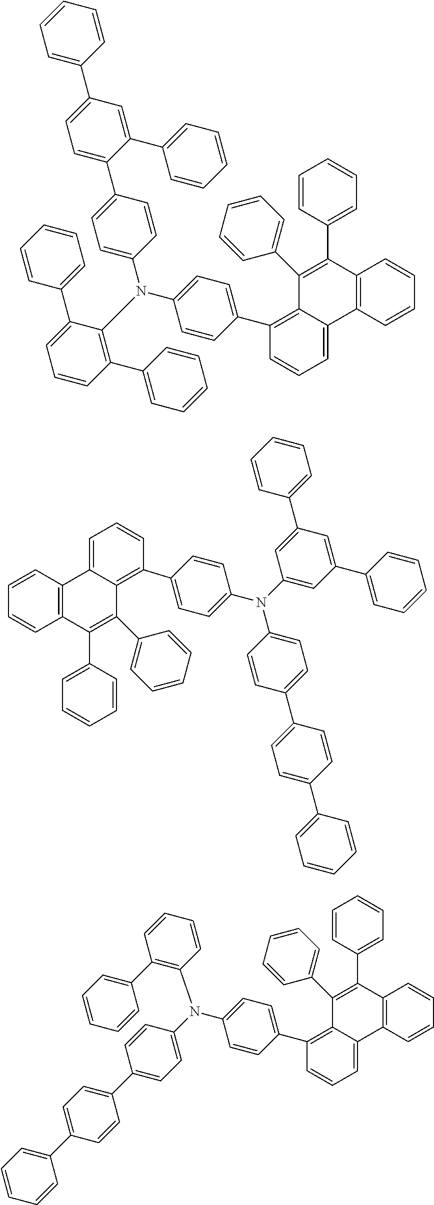

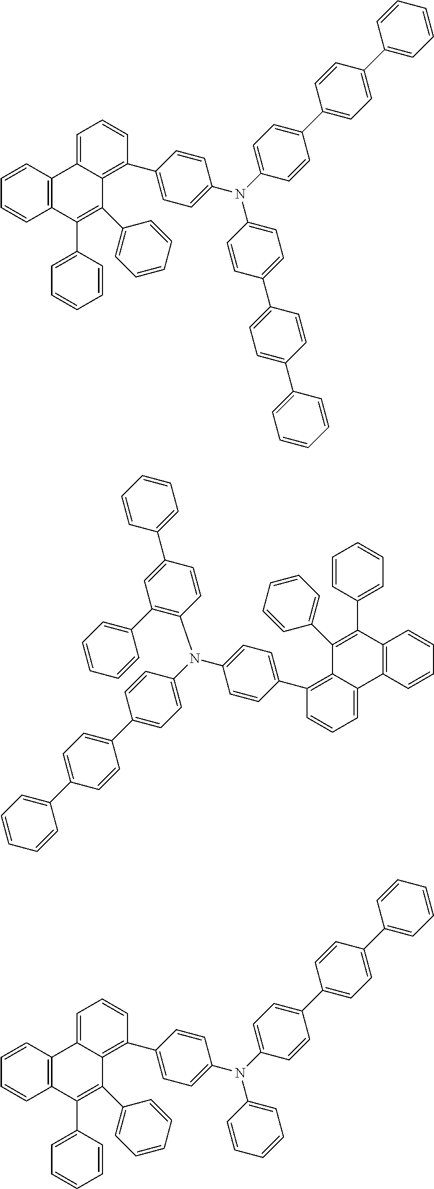 [SMILES: c1ccc(-c2ccc(-c3ccc(N(c4ccc(-c5ccc(-c6ccccc6)cc5)cc4)c4ccc(-c5cccc6c5c(-c5ccccc5)c(-c5ccccc5)c5ccccc56)cc4)cc3)cc2)cc1 ; c1ccc(-c2ccc(-c3ccc(N(c4ccc(-c5cccc6c5c(-c5ccccc5)c(-c5ccccc5)c5ccccc56)cc4)c4ccc(-c5ccccc5)cc4-c4ccccc4)cc3)cc2)cc1 ; c1ccc(-c2ccc(-c3ccc(N(c4ccccc4)c4ccc(-c5cccc6c5c(-c5ccccc5)c(-c5ccccc5)c5ccccc56)cc4)cc3)cc2)cc1]

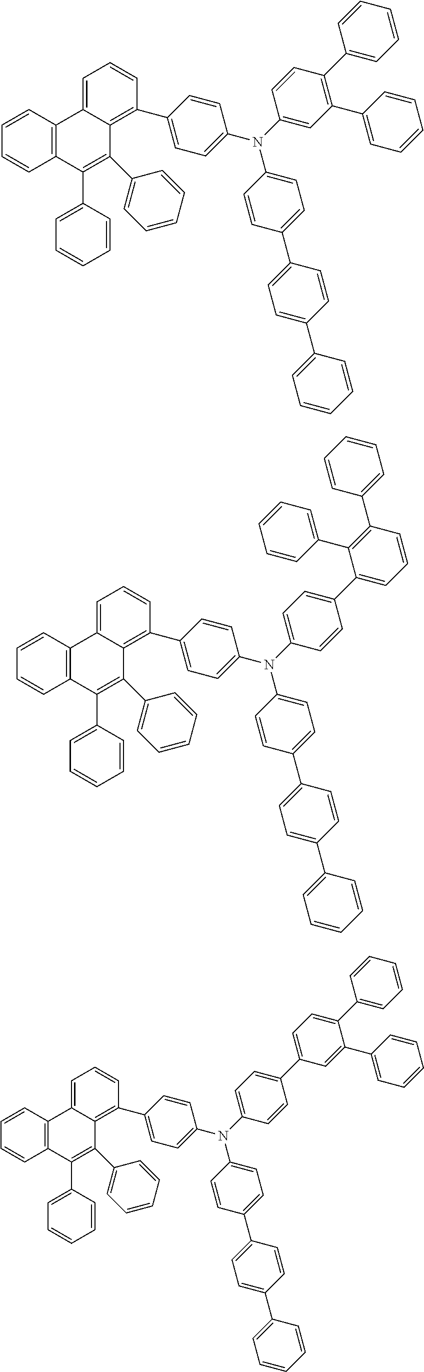

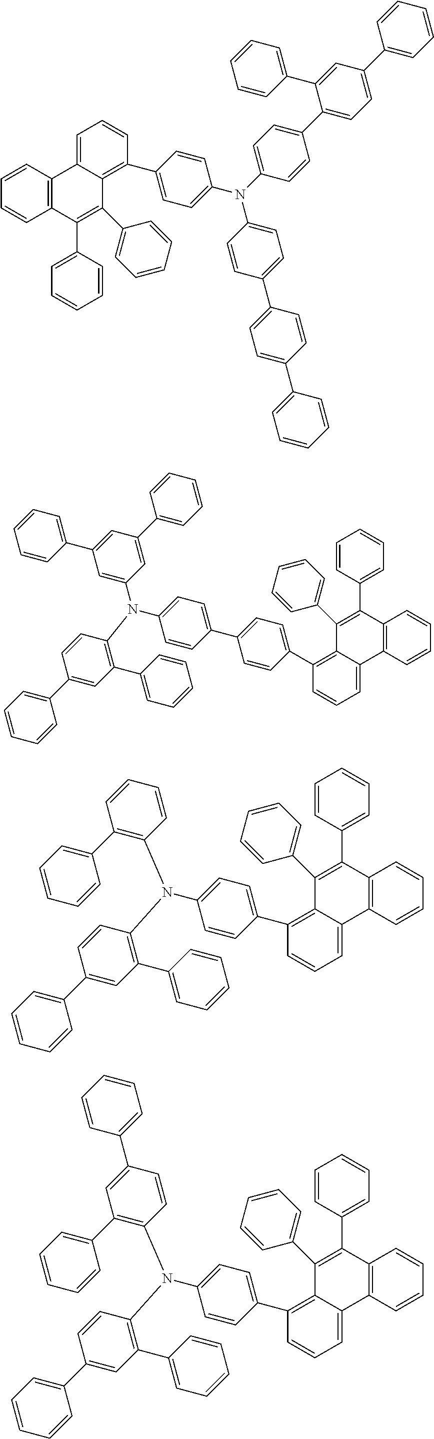 [SMILES: c1ccc(-c2ccc(-c3ccc(N(c4ccc(-c5ccc(-c6ccccc6)cc5-c5ccccc5)cc4)c4ccc(-c5cccc6c5c(-c5ccccc5)c(-c5ccccc5)c5ccccc56)cc4)cc3)cc2)cc1 ; c1ccc(-c2cc(-c3ccccc3)cc(N(c3ccc(-c4ccc(-c5cccc6c5c(-c5ccccc5)c(-c5ccccc5)c5ccccc56)cc4)cc3)c3ccc(-c4ccccc4)cc3-c3ccccc3)c2)cc1 ; c1ccc(-c2ccc(N(c3ccc(-c4cccc5c4c(-c4ccccc4)c(-c4ccccc4)c4ccccc45)cc3)c3ccccc3-c3ccccc3)c(-c3ccccc3)c2)cc1 ; c1ccc(-c2ccc(N(c3ccc(-c4cccc5c4c(-c4ccccc4)c(-c4ccccc4)c4ccccc45)cc3)c3ccc(-c4ccccc4)cc3-c3ccccc3)c(-c3ccccc3)c2)cc1]

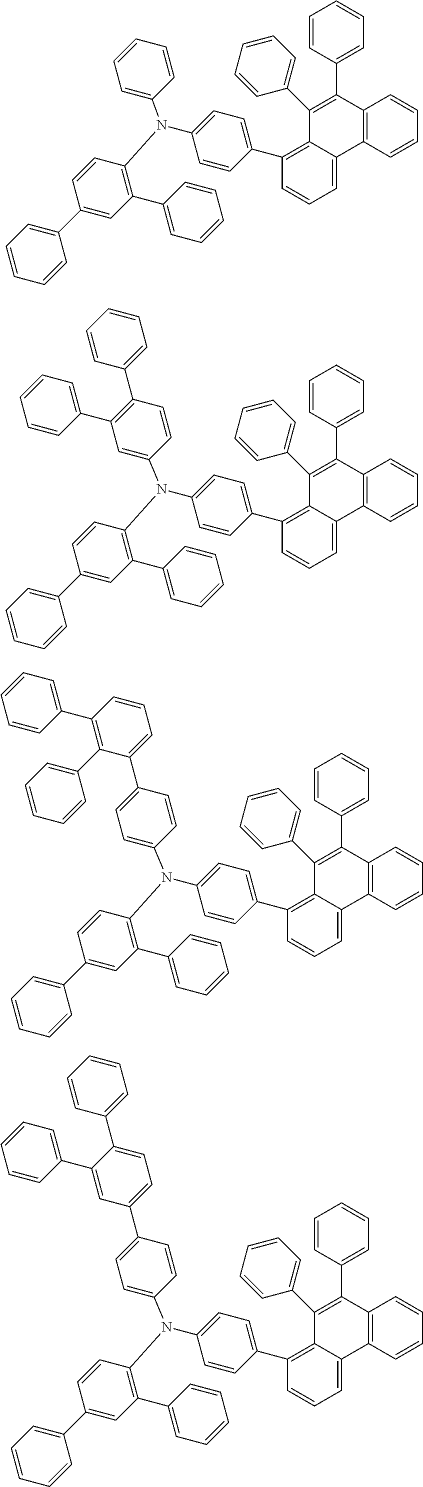

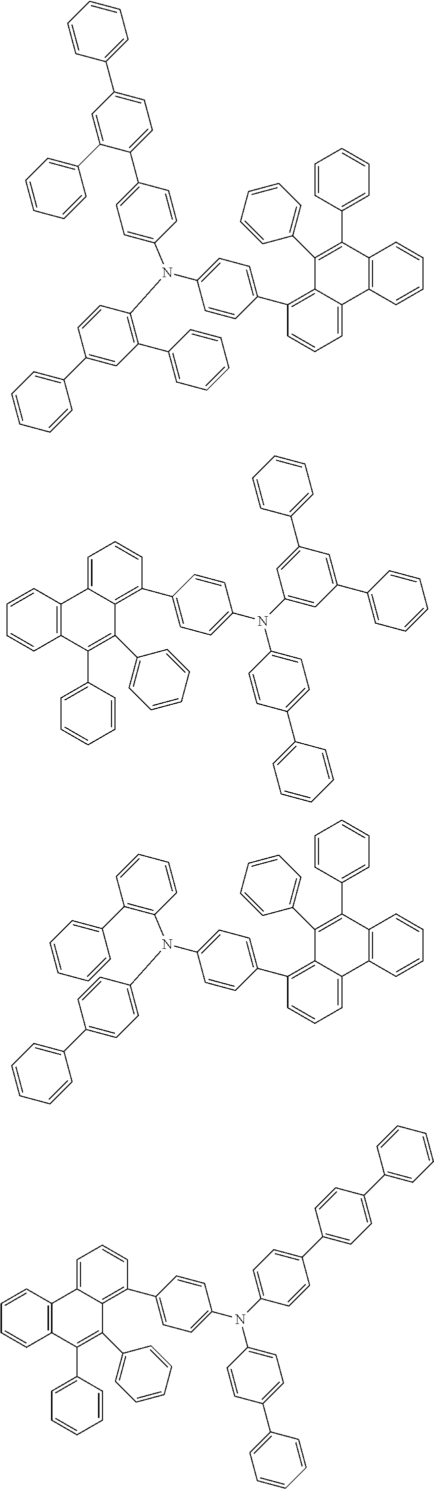

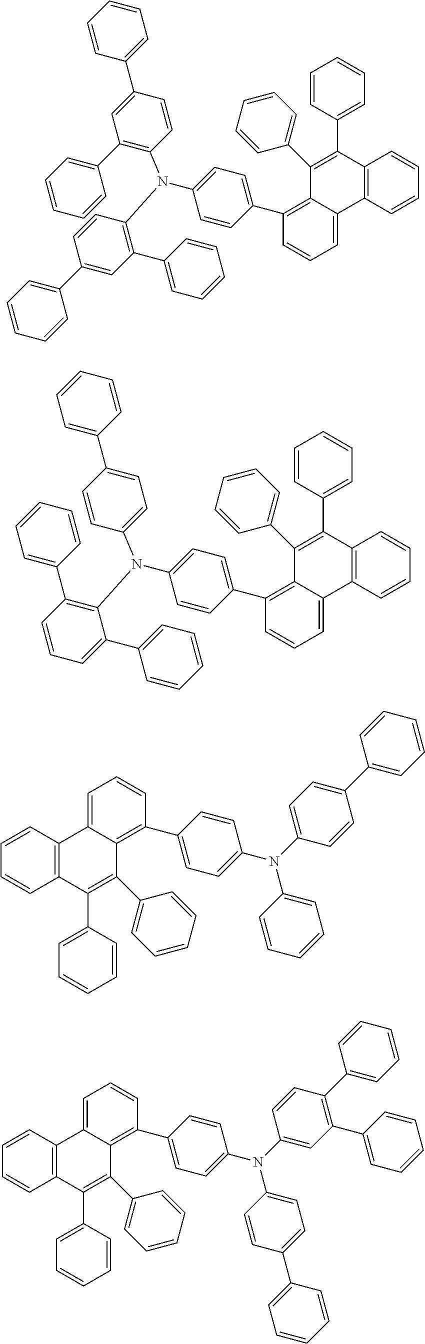 [SMILES: c1ccc(-c2ccc(N(c3ccc(-c4cccc5c4c(-c4ccccc4)c(-c4ccccc4)c4ccccc45)cc3)c3ccc(-c4ccccc4)cc3-c3ccccc3)c(-c3ccccc3)c2)cc1 ; c1ccc(-c2ccc(N(c3ccc(-c4cccc5c4c(-c4ccccc4)c(-c4ccccc4)c4ccccc45)cc3)c3c(-c4ccccc4)cccc3-c3ccccc3)cc2)cc1 ; c1ccc(-c2ccc(N(c3ccccc3)c3ccc(-c4cccc5c4c(-c4ccccc4)c(-c4ccccc4)c4ccccc45)cc3)cc2)cc1 ; c1ccc(-c2ccc(N(c3ccc(-c4cccc5c4c(-c4ccccc4)c(-c4ccccc4)c4ccccc45)cc3)c3ccc(-c4ccccc4)c(-c4ccccc4)c3)cc2)cc1]

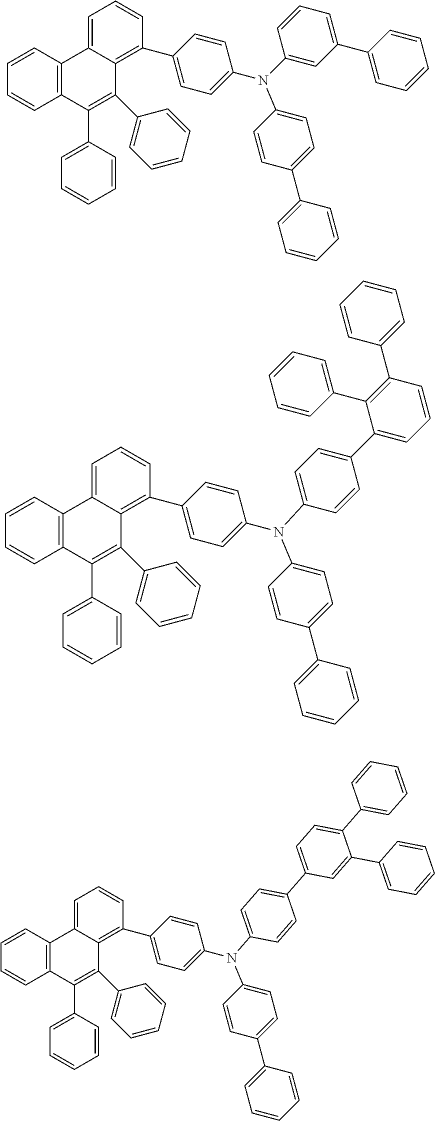 [SMILES: c1ccc(-c2ccc(N(c3ccc(-c4cccc5c4c(-c4ccccc4)c(-c4ccccc4)c4ccccc45)cc3)c3cccc(-c4ccccc4)c3)cc2)cc1 ; c1ccc(-c2ccc(N(c3ccc(-c4cccc(-c5ccccc5)c4-c4ccccc4)cc3)c3ccc(-c4cccc5c4c(-c4ccccc4)c(-c4ccccc4)c4ccccc45)cc3)cc2)cc1 ; c1ccc(-c2ccc(N(c3ccc(-c4ccc(-c5ccccc5)c(-c5ccccc5)c4)cc3)c3ccc(-c4cccc5c4c(-c4ccccc4)c(-c4ccccc4)c4ccccc45)cc3)cc2)cc1]

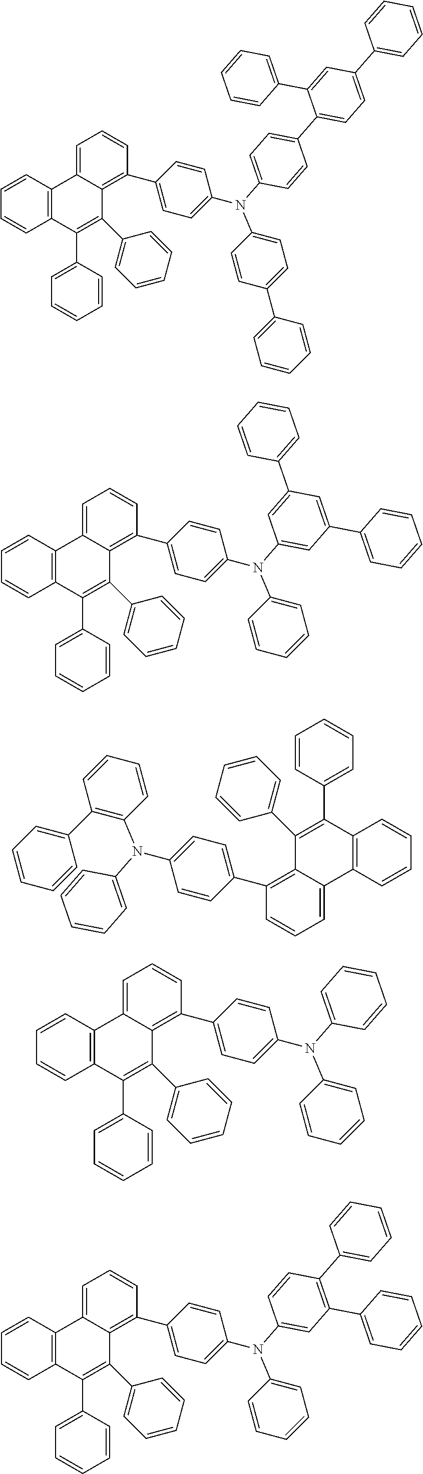

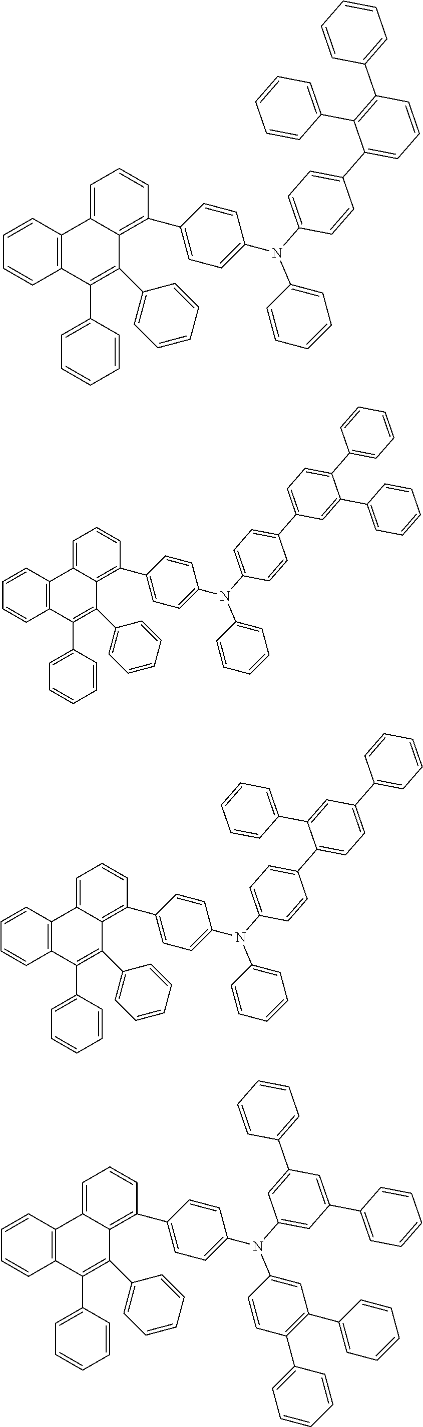 [SMILES: c1ccc(-c2cccc(-c3ccc(N(c4ccccc4)c4ccc(-c5cccc6c5c(-c5ccccc5)c(-c5ccccc5)c5ccccc56)cc4)cc3)c2-c2ccccc2)cc1 ; c1ccc(-c2ccc(-c3ccc(N(c4ccccc4)c4ccc(-c5cccc6c5c(-c5ccccc5)c(-c5ccccc5)c5ccccc56)cc4)cc3)cc2-c2ccccc2)cc1 ; c1ccc(-c2ccc(-c3ccc(N(c4ccccc4)c4ccc(-c5cccc6c5c(-c5ccccc5)c(-c5ccccc5)c5ccccc56)cc4)cc3)c(-c3ccccc3)c2)cc1 ; c1ccc(-c2cc(-c3ccccc3)cc(N(c3ccc(-c4cccc5c4c(-c4ccccc4)c(-c4ccccc4)c4ccccc45)cc3)c3ccc(-c4ccccc4)c(-c4ccccc4)c3)c2)cc1]

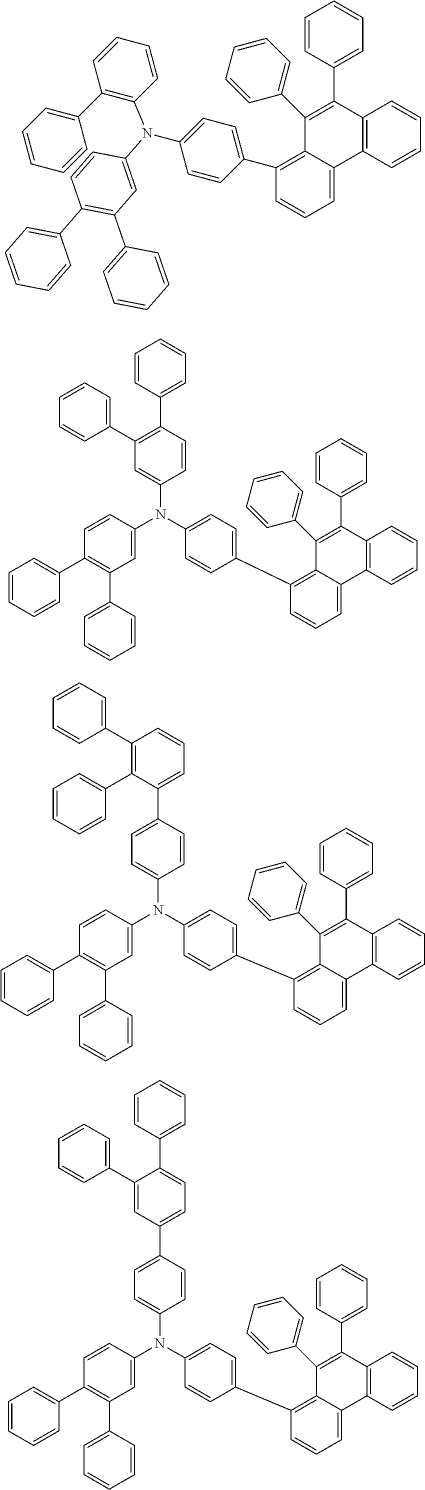

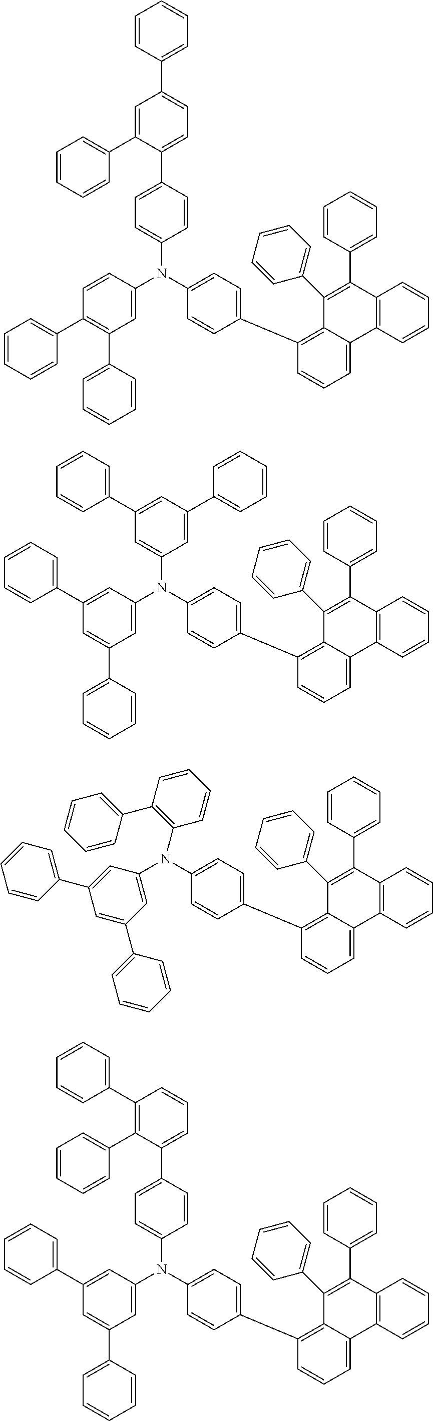

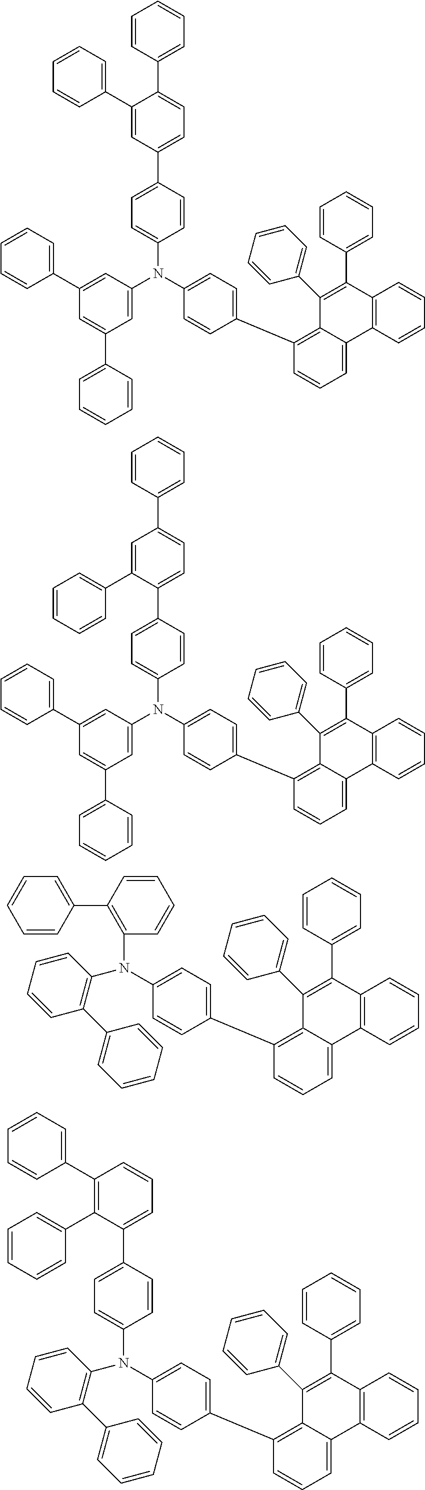

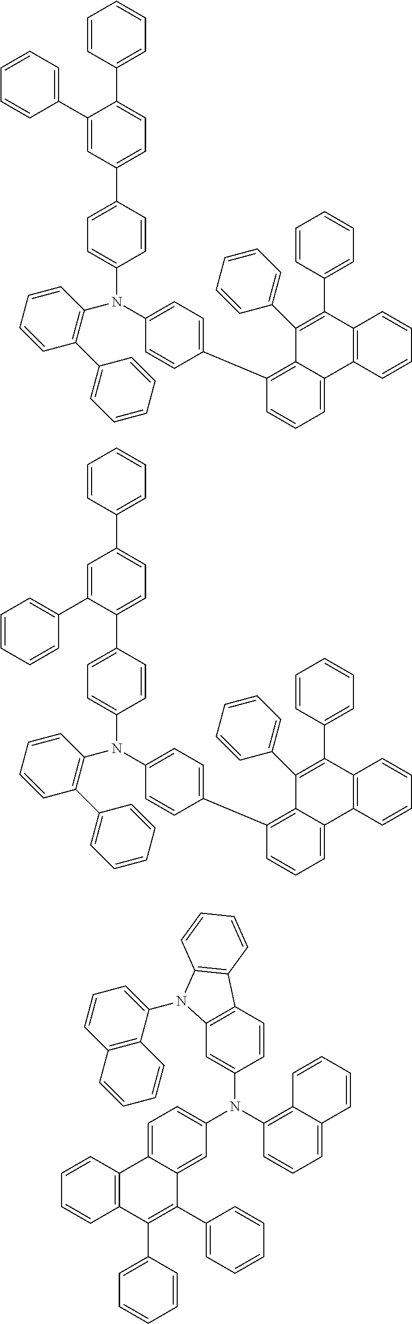

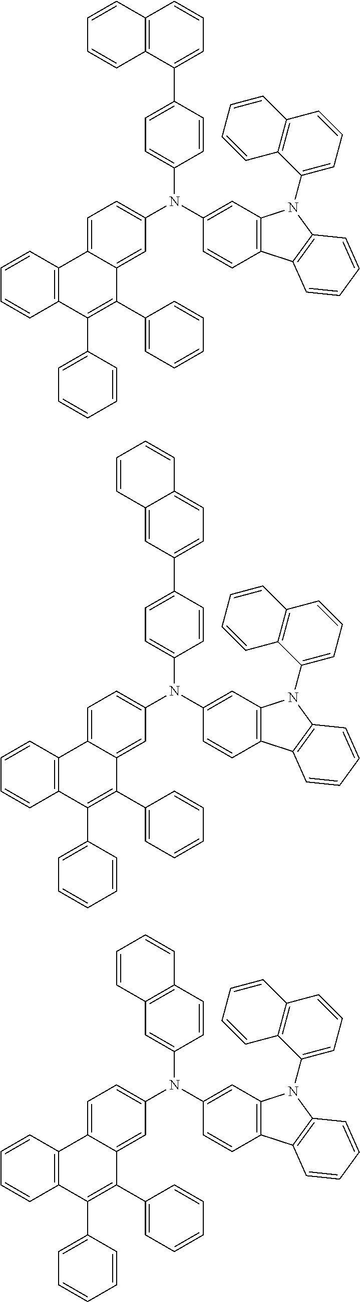 [SMILES: c1ccc(-c2c(-c3ccccc3)c3cc(N(c4ccc(-c5cccc6ccccc56)cc4)c4ccc5c6ccccc6n(-c6cccc7ccccc67)c5c4)ccc3c3ccccc23)cc1 ; c1ccc(-c2c(-c3ccccc3)c3cc(N(c4ccc(-c5ccc6ccccc6c5)cc4)c4ccc5c6ccccc6n(-c6cccc7ccccc67)c5c4)ccc3c3ccccc23)cc1 ; c1ccc(-c2c(-c3ccccc3)c3cc(N(c4ccc5ccccc5c4)c4ccc5c6ccccc6n(-c6cccc7ccccc67)c5c4)ccc3c3ccccc23)cc1]

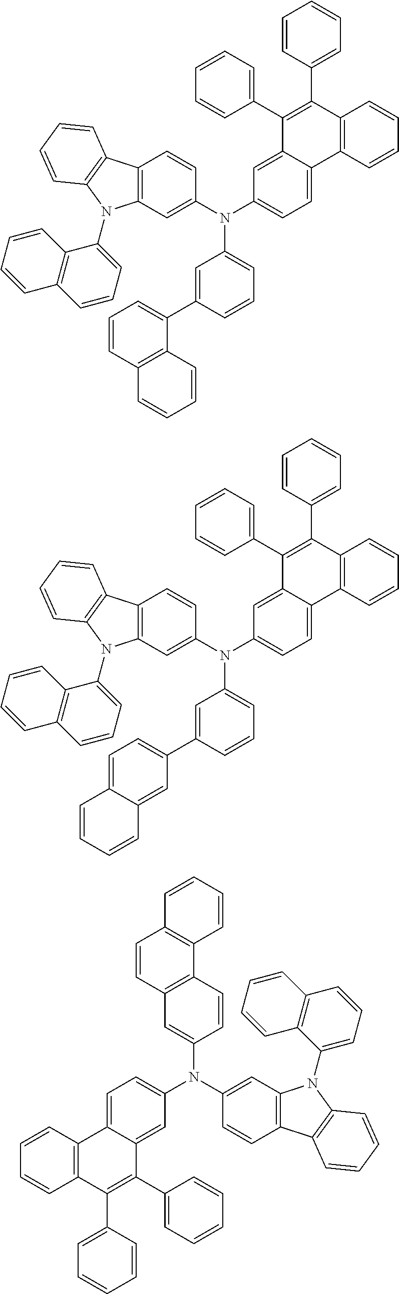 [SMILES: c1ccc(-c2c(-c3ccccc3)c3cc(N(c4cccc(-c5cccc6ccccc56)c4)c4ccc5c6ccccc6n(-c6cccc7ccccc67)c5c4)ccc3c3ccccc23)cc1 ; c1ccc(-c2c(-c3ccccc3)c3cc(N(c4cccc(-c5ccc6ccccc6c5)c4)c4ccc5c6ccccc6n(-c6cccc7ccccc67)c5c4)ccc3c3ccccc23)cc1 ; c1ccc(-c2c(-c3ccccc3)c3cc(N(c4ccc5c(ccc6ccccc65)c4)c4ccc5c6ccccc6n(-c6cccc7ccccc67)c5c4)ccc3c3ccccc23)cc1]

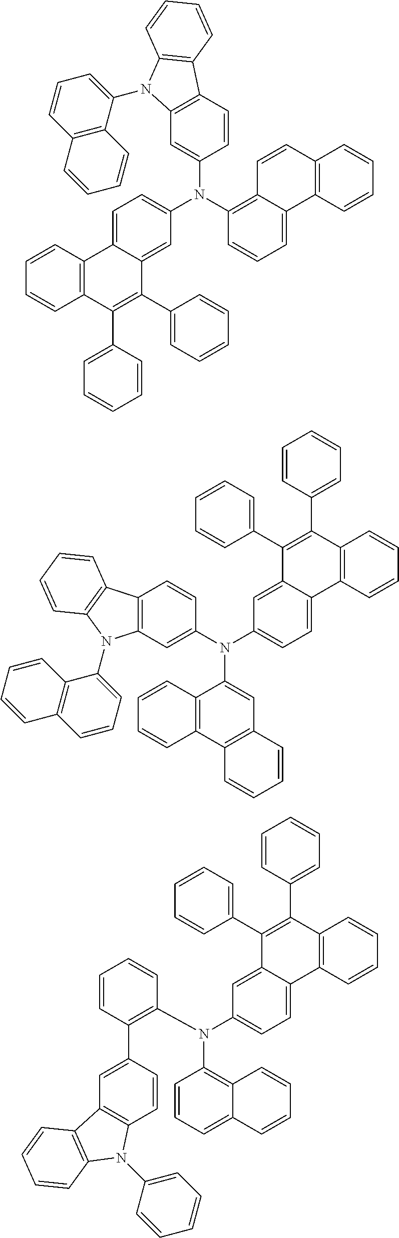

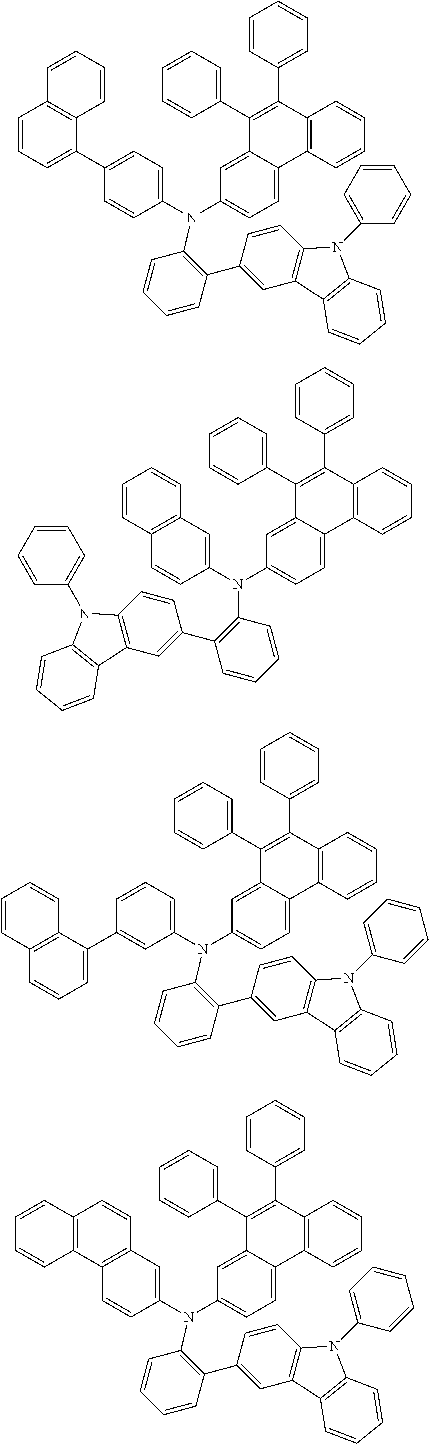

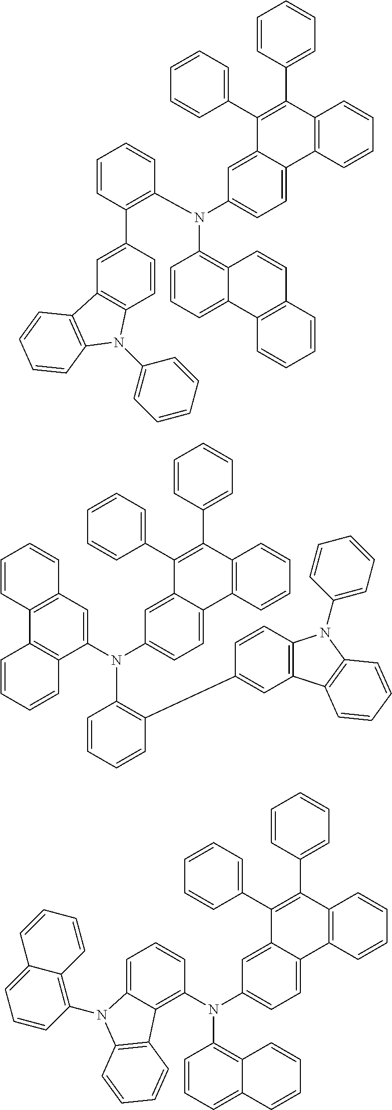

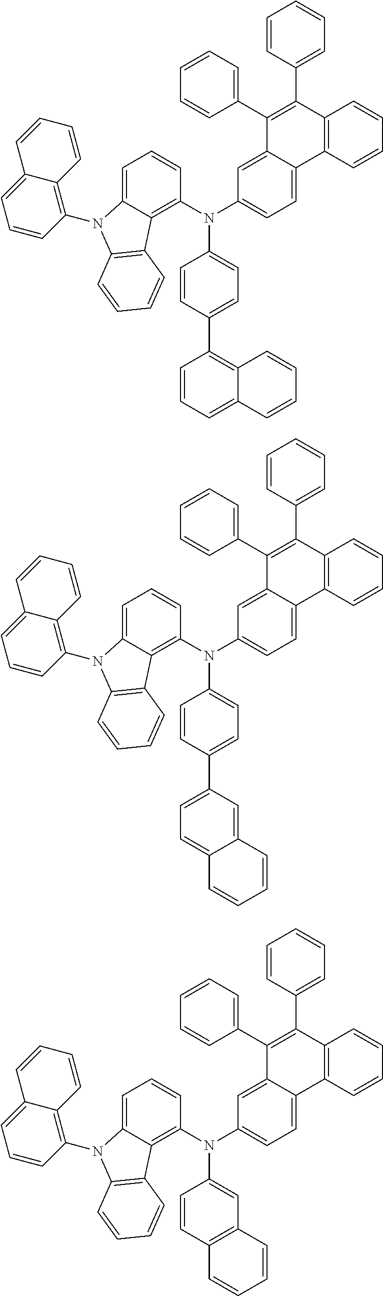

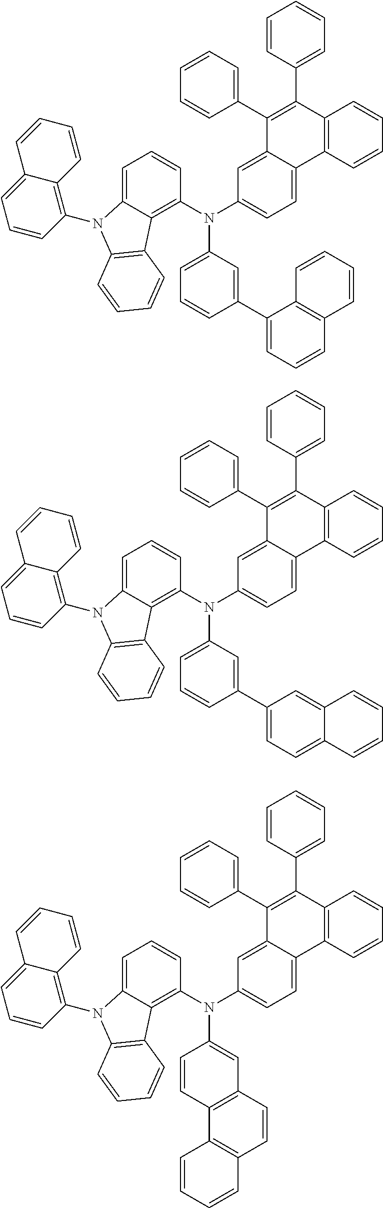

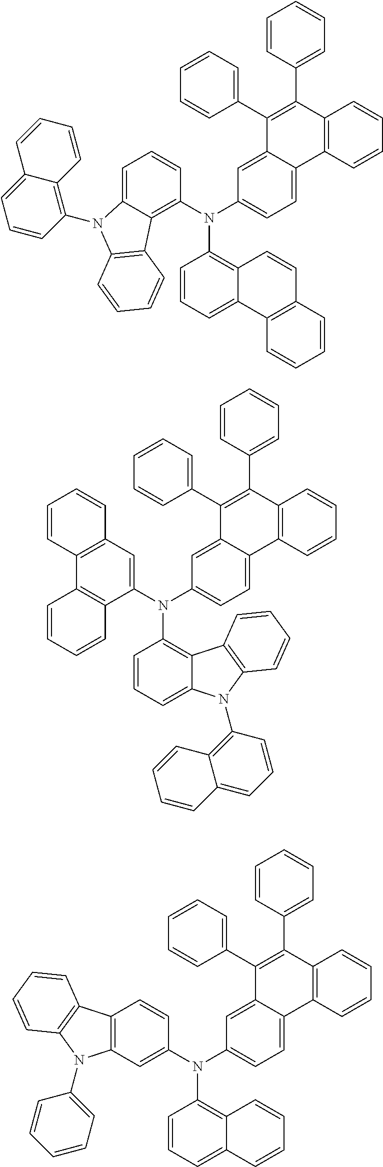

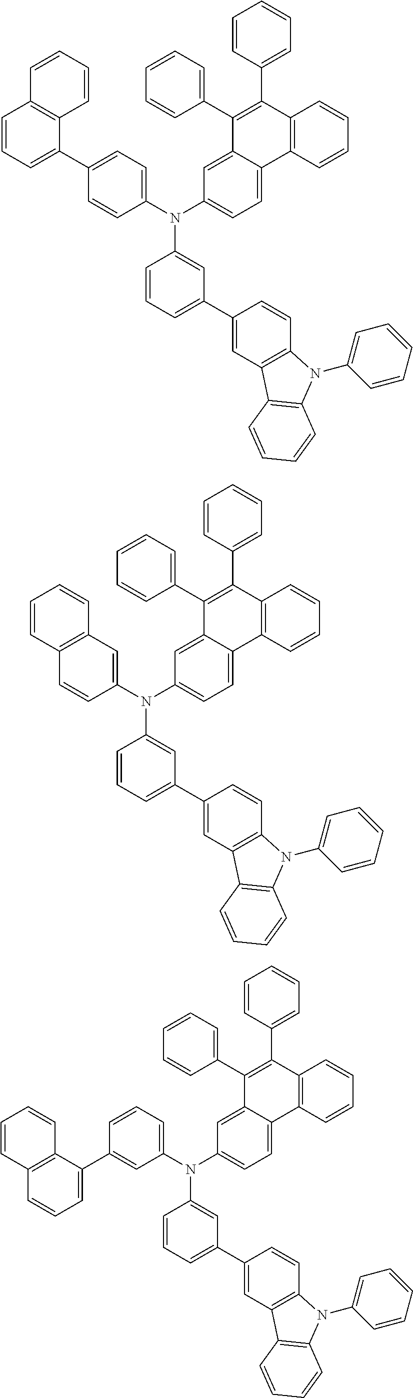

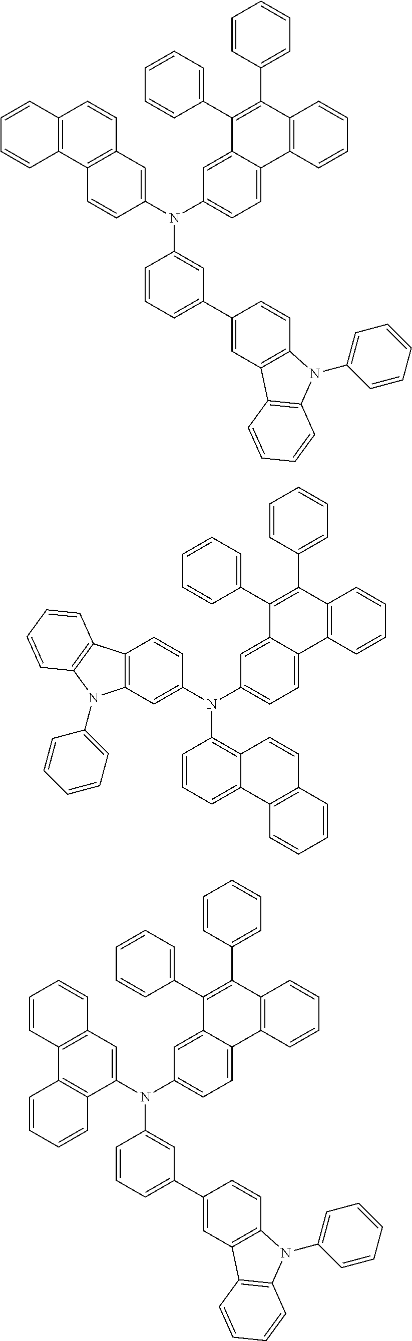

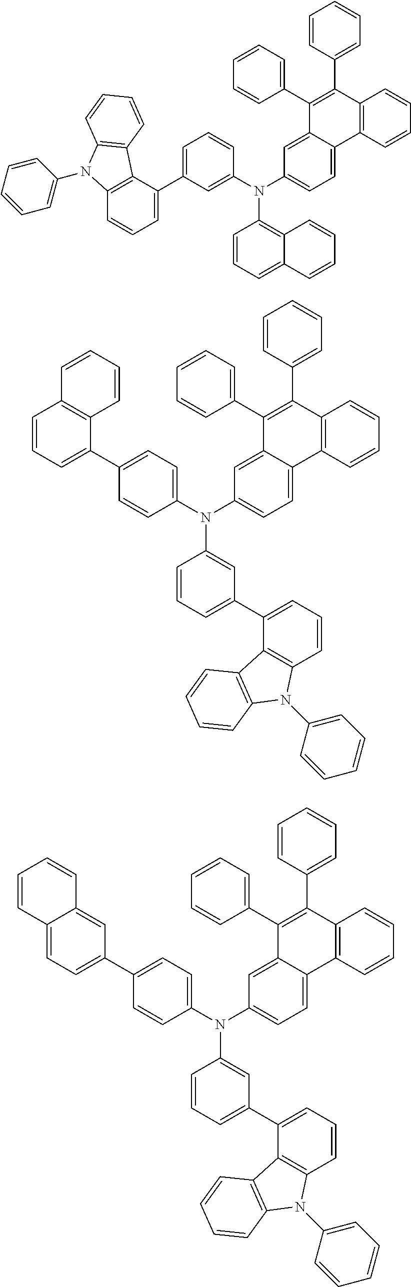

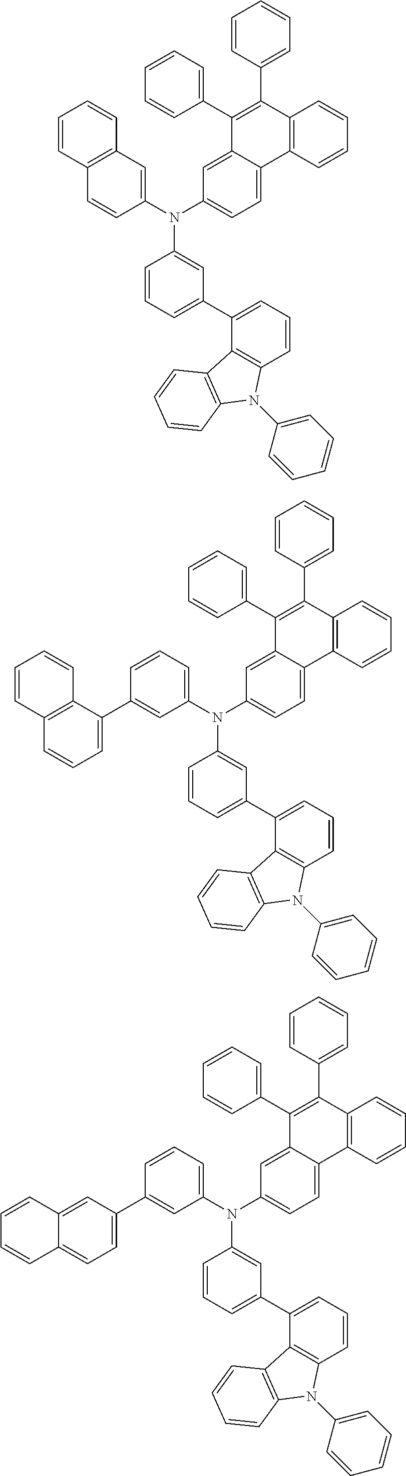

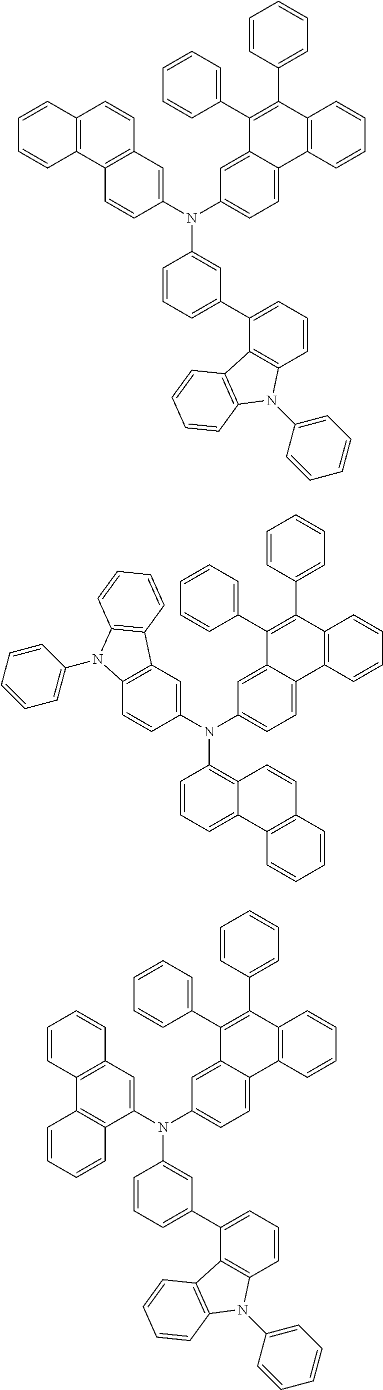

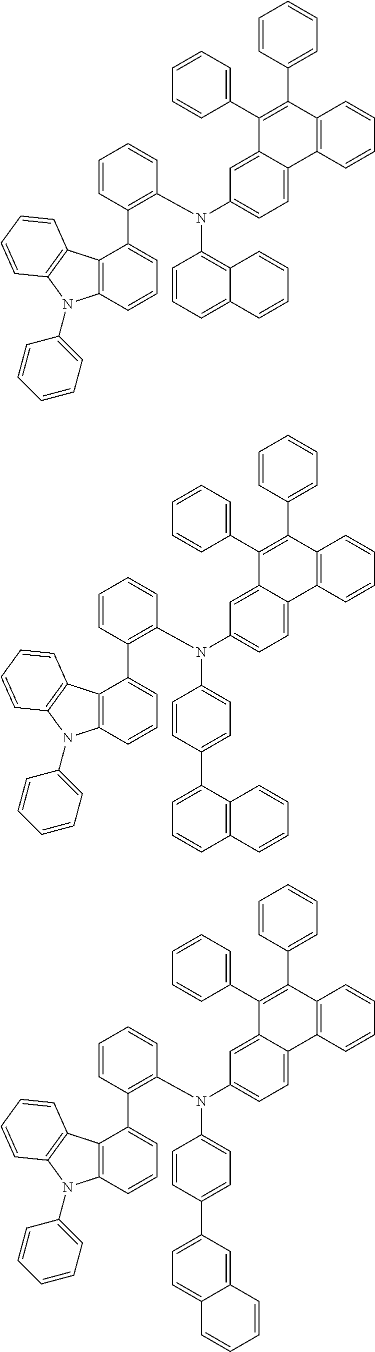

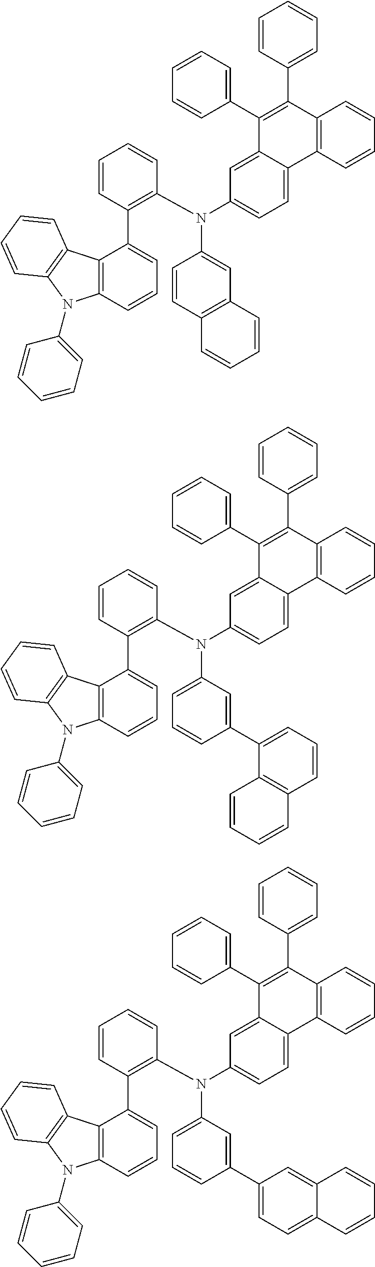

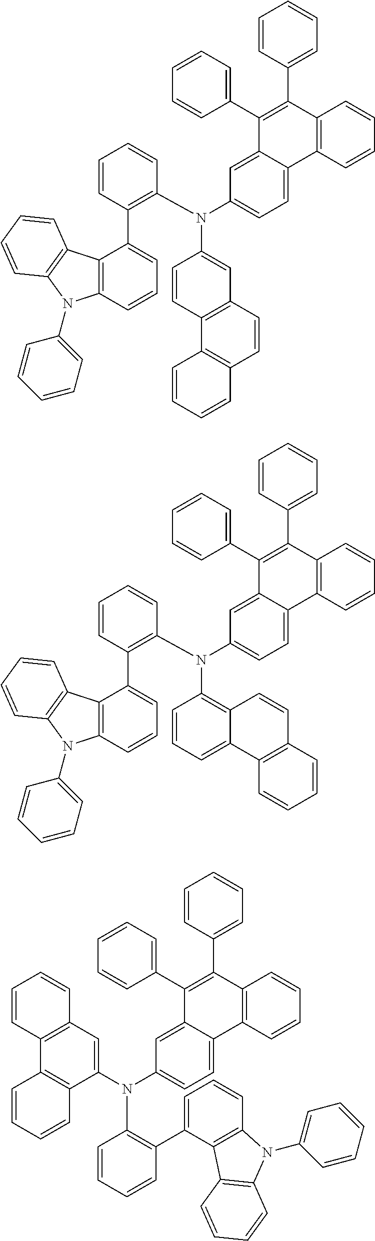 [SMILES: c1ccc(-c2c(-c3ccccc3)c3cc(N(c4ccc5c(ccc6ccccc65)c4)c4ccccc4-c4cccc5c4c4ccccc4n5-c4ccccc4)ccc3c3ccccc23)cc1 ; c1ccc(-c2c(-c3ccccc3)c3cc(N(c4ccccc4-c4cccc5c4c4ccccc4n5-c4ccccc4)c4cccc5c4ccc4ccccc45)ccc3c3ccccc23)cc1 ; c1ccc(-c2c(-c3ccccc3)c3cc(N(c4ccccc4-c4cccc5c4c4ccccc4n5-c4ccccc4)c4cc5ccccc5c5ccccc45)ccc3c3ccccc23)cc1]

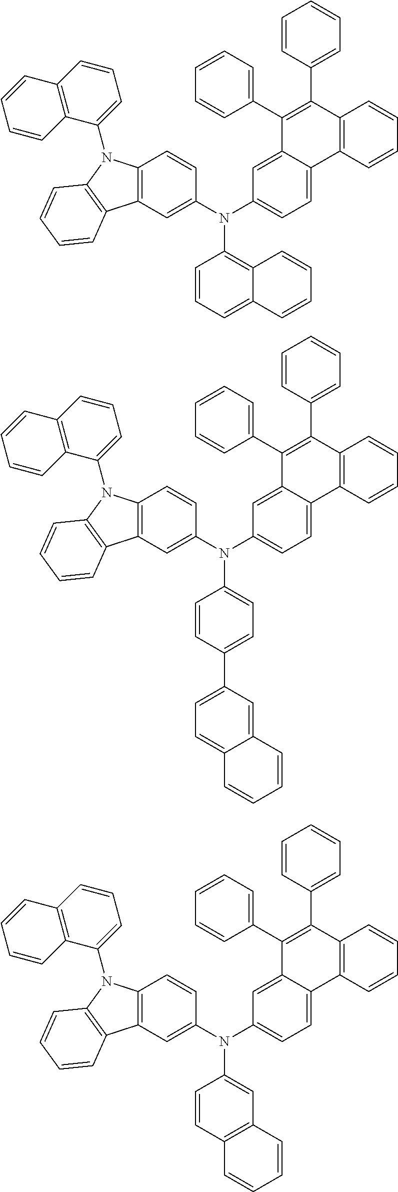

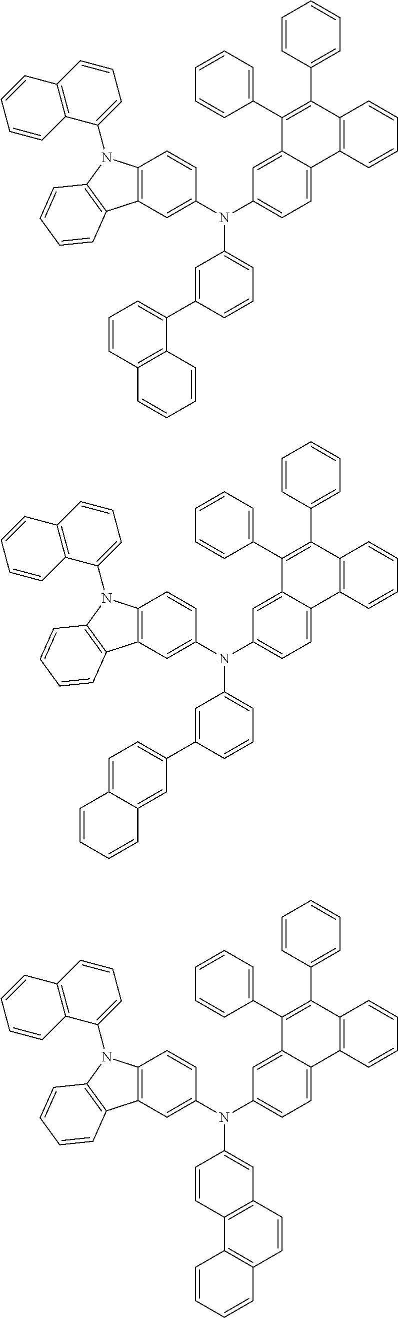 [SMILES: c1ccc(-c2c(-c3ccccc3)c3cc(N(c4cccc(-c5cccc6ccccc56)c4)c4ccc5c(c4)c4ccccc4n5-c4cccc5ccccc45)ccc3c3ccccc23)cc1 ; c1ccc(-c2c(-c3ccccc3)c3cc(N(c4cccc(-c5ccc6ccccc6c5)c4)c4ccc5c(c4)c4ccccc4n5-c4cccc5ccccc45)ccc3c3ccccc23)cc1 ; c1ccc(-c2c(-c3ccccc3)c3cc(N(c4ccc5c(ccc6ccccc65)c4)c4ccc5c(c4)c4ccccc4n5-c4cccc5ccccc45)ccc3c3ccccc23)cc1]

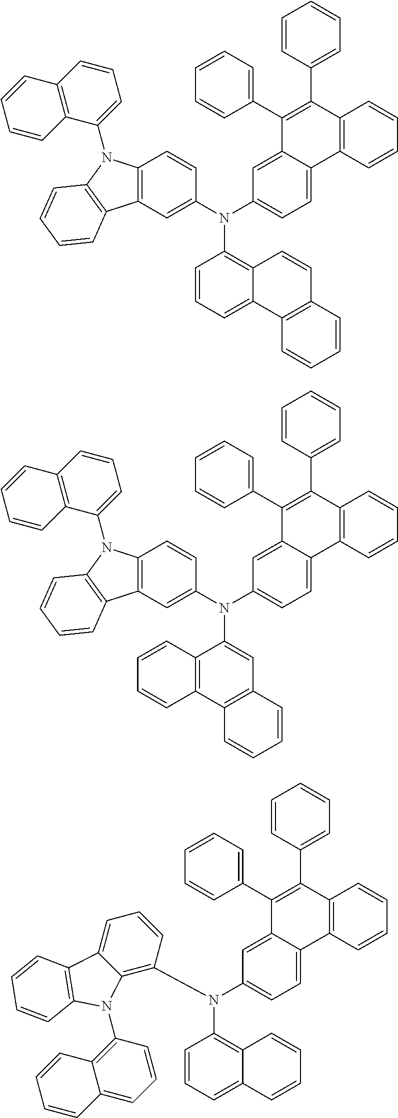

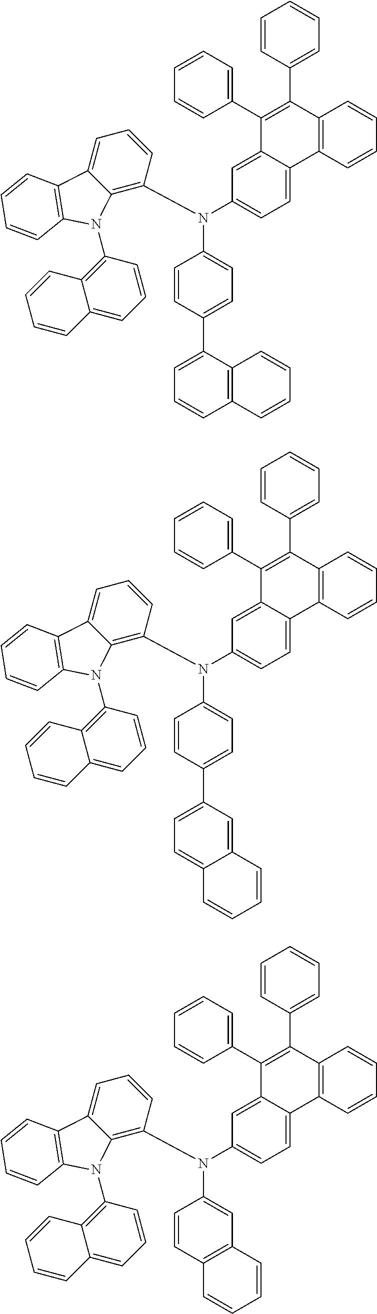

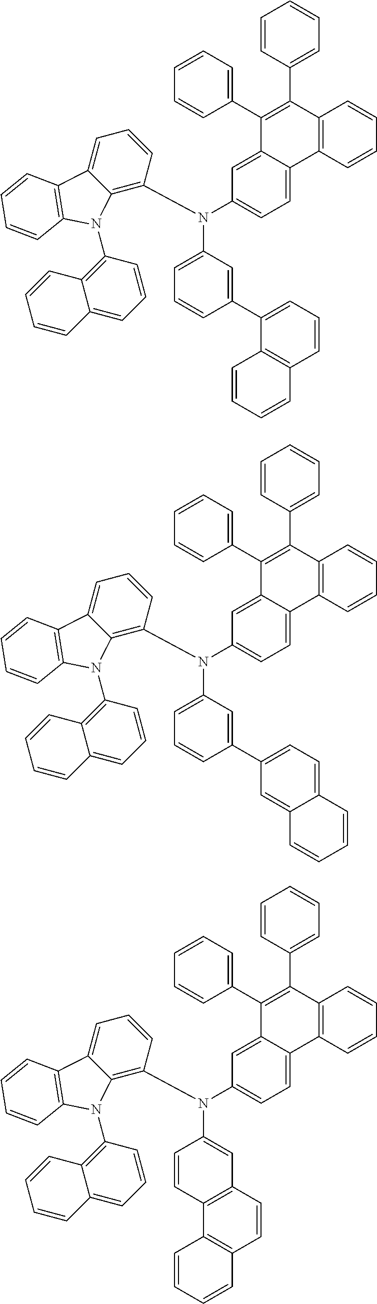

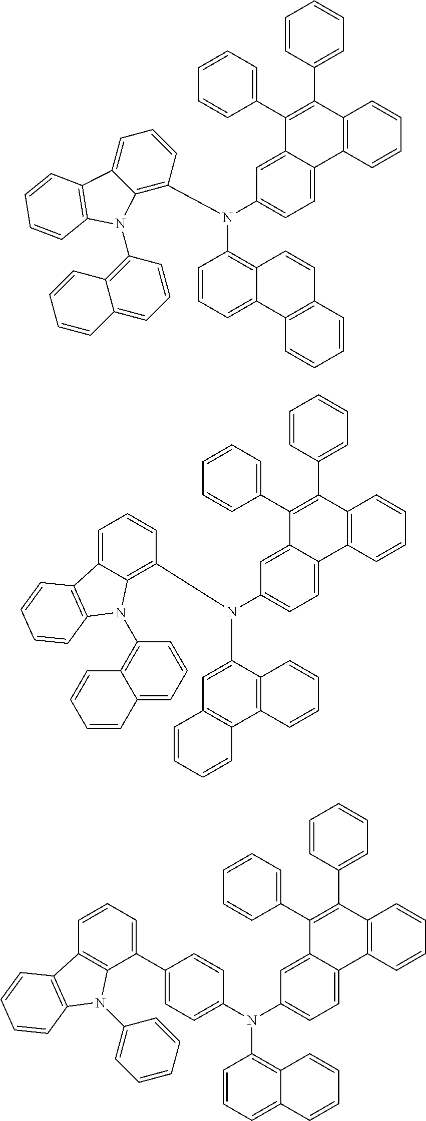

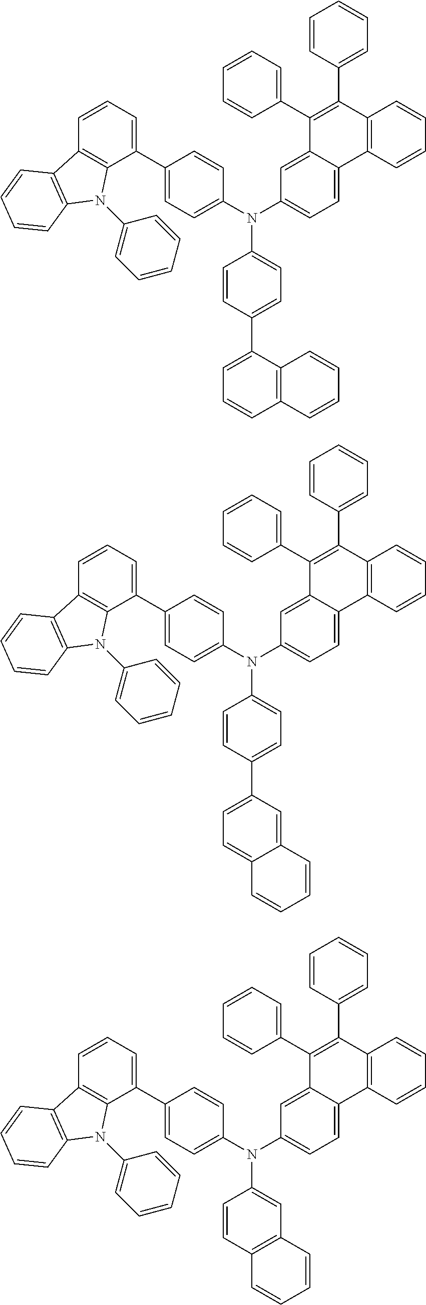

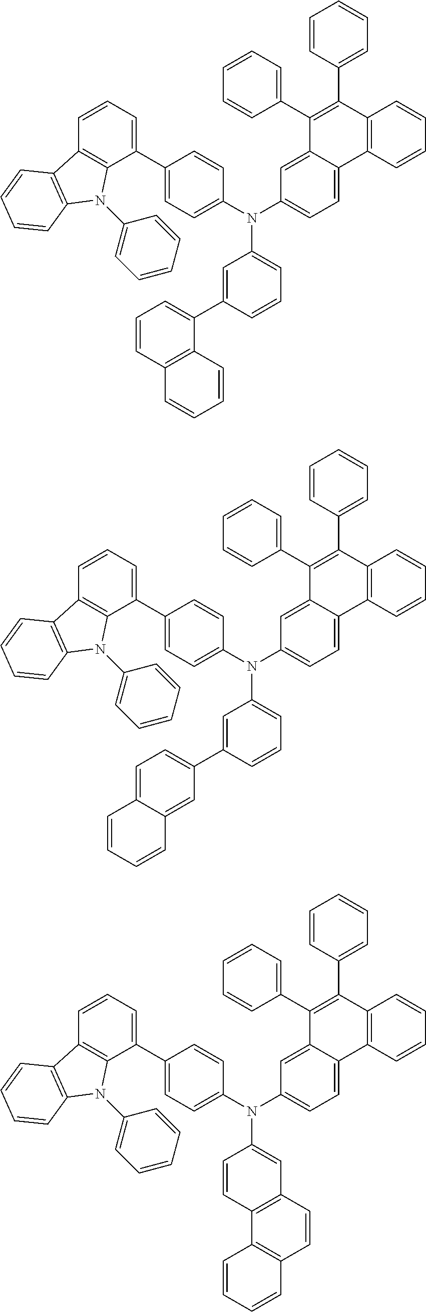 [SMILES: c1ccc(-c2c(-c3ccccc3)c3cc(N(c4ccc(-c5cccc6c7ccccc7n(-c7ccccc7)c56)cc4)c4cccc(-c5cccc6ccccc56)c4)ccc3c3ccccc23)cc1 ; c1ccc(-c2c(-c3ccccc3)c3cc(N(c4ccc(-c5cccc6c7ccccc7n(-c7ccccc7)c56)cc4)c4cccc(-c5ccc6ccccc6c5)c4)ccc3c3ccccc23)cc1 ; c1ccc(-c2c(-c3ccccc3)c3cc(N(c4ccc(-c5cccc6c7ccccc7n(-c7ccccc7)c56)cc4)c4ccc5c(ccc6ccccc65)c4)ccc3c3ccccc23)cc1]

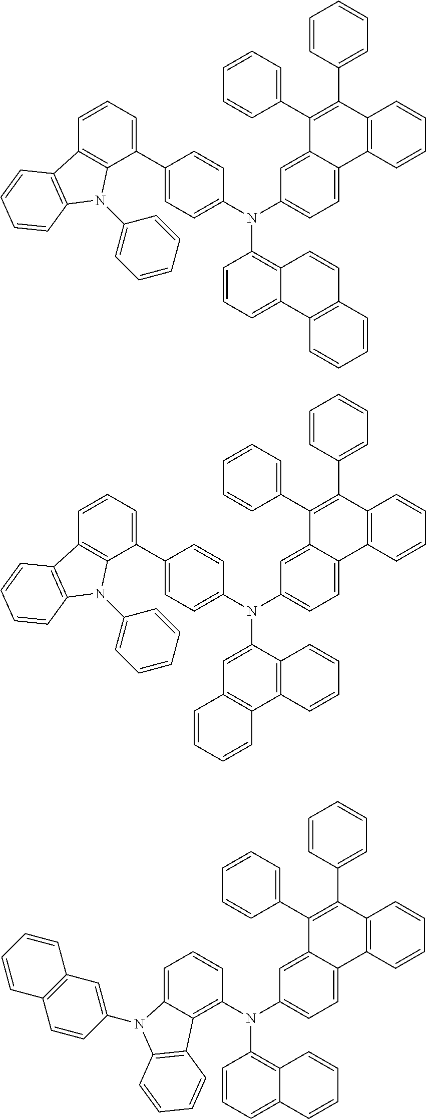

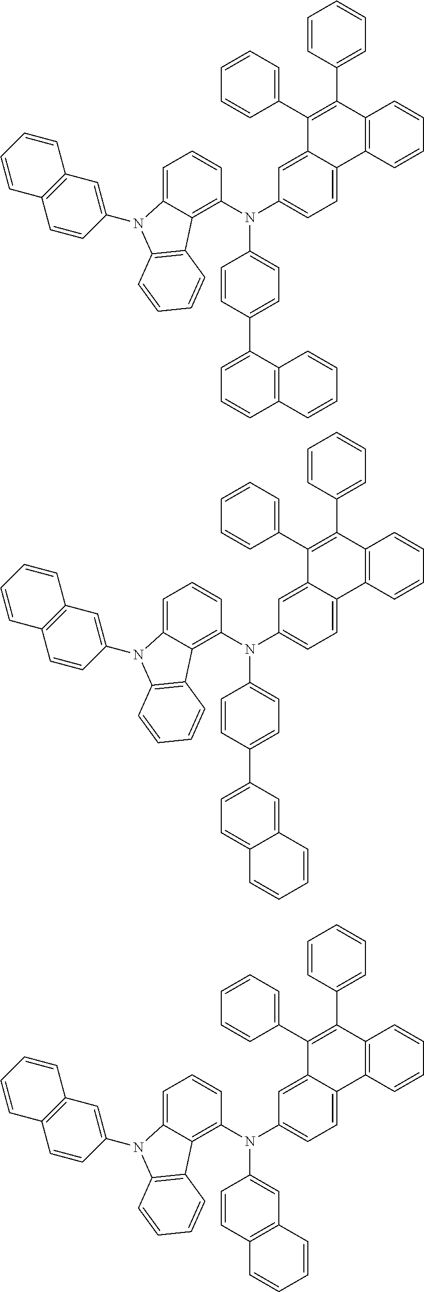

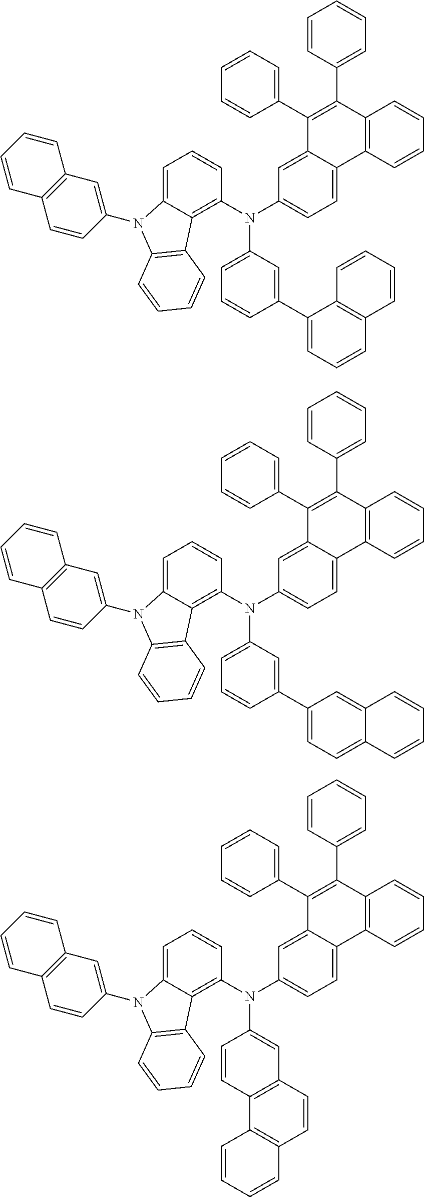

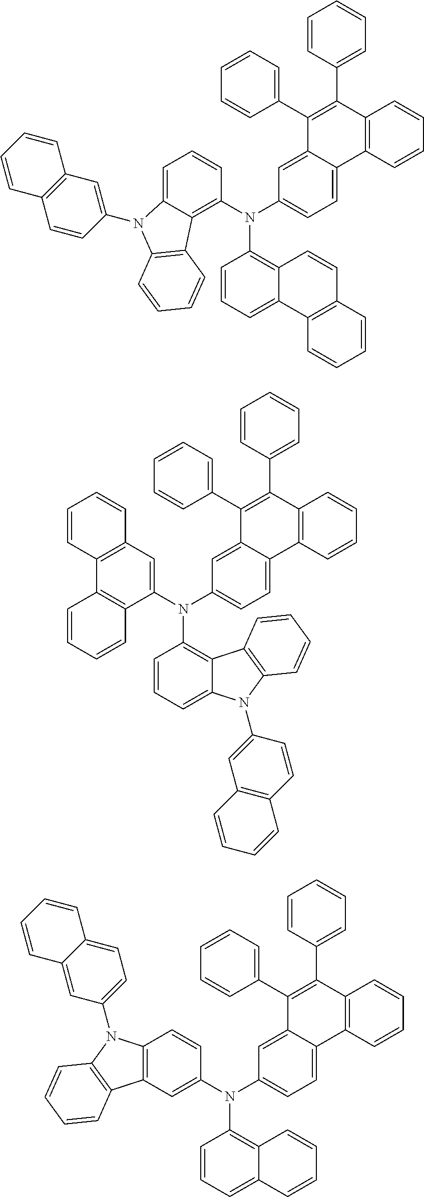 [SMILES: c1ccc(-c2c(-c3ccccc3)c3cc(N(c4cccc5c4ccc4ccccc45)c4cccc5c4c4ccccc4n5-c4ccc5ccccc5c4)ccc3c3ccccc23)cc1 ; c1ccc(-c2c(-c3ccccc3)c3cc(N(c4cc5ccccc5c5ccccc45)c4cccc5c4c4ccccc4n5-c4ccc5ccccc5c4)ccc3c3ccccc23)cc1 ; c1ccc(-c2c(-c3ccccc3)c3cc(N(c4ccc5c(c4)c4ccccc4n5-c4ccc5ccccc5c4)c4cccc5ccccc45)ccc3c3ccccc23)cc1]

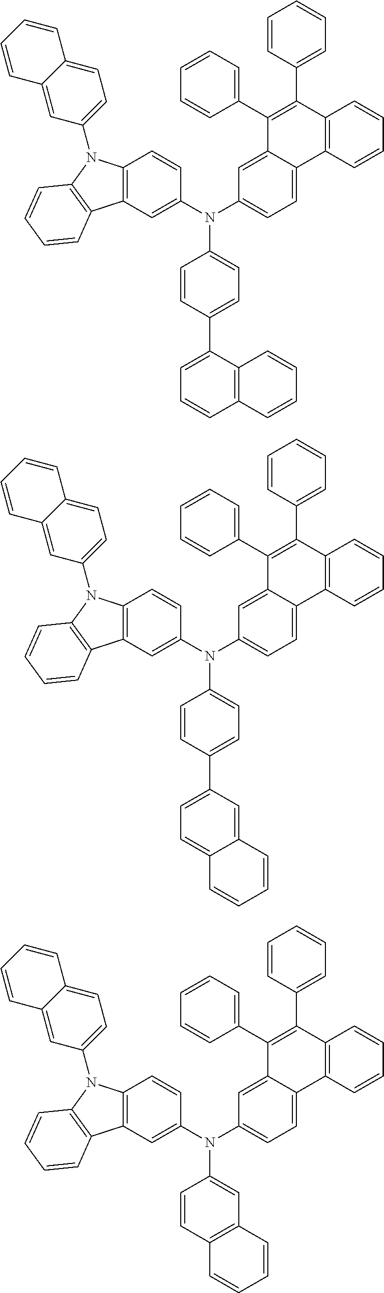

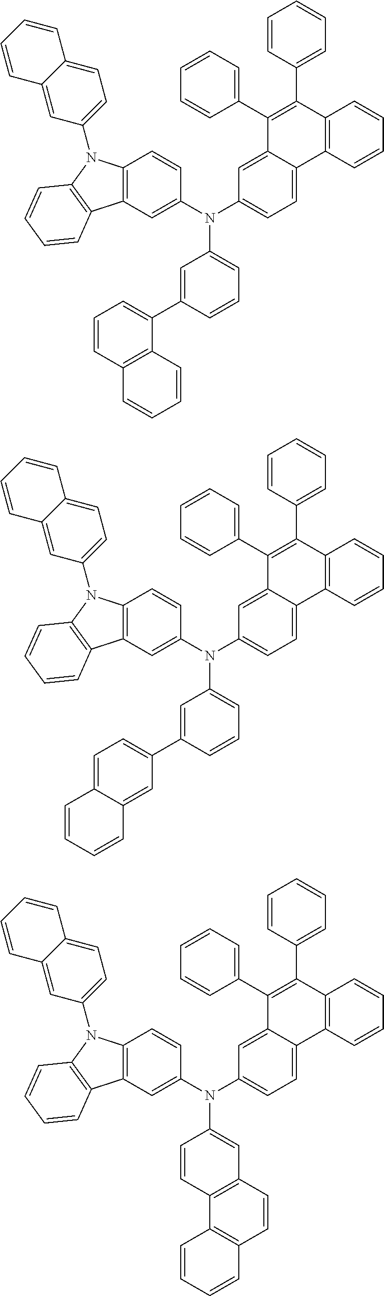 [SMILES: c1ccc(-c2c(-c3ccccc3)c3cc(N(c4cccc(-c5cccc6ccccc56)c4)c4ccc5c(c4)c4ccccc4n5-c4ccc5ccccc5c4)ccc3c3ccccc23)cc1 ; c1ccc(-c2c(-c3ccccc3)c3cc(N(c4cccc(-c5ccc6ccccc6c5)c4)c4ccc5c(c4)c4ccccc4n5-c4ccc5ccccc5c4)ccc3c3ccccc23)cc1 ; c1ccc(-c2c(-c3ccccc3)c3cc(N(c4ccc5c(ccc6ccccc65)c4)c4ccc5c(c4)c4ccccc4n5-c4ccc5ccccc5c4)ccc3c3ccccc23)cc1]

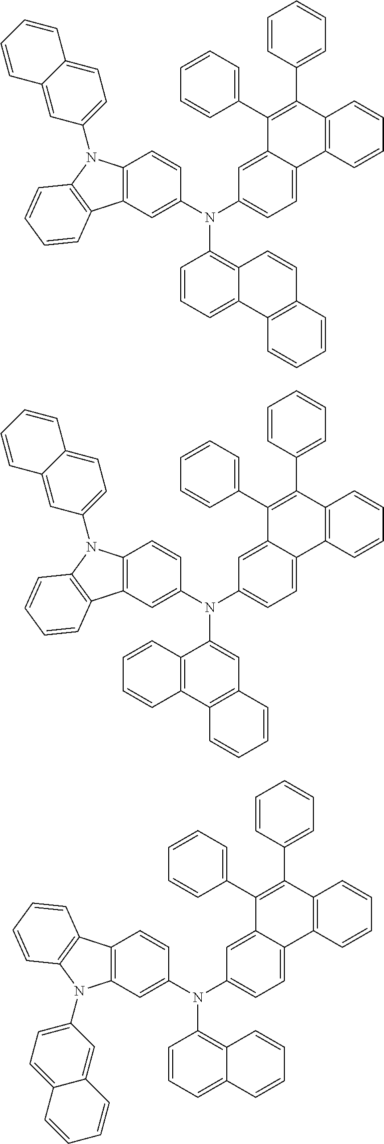

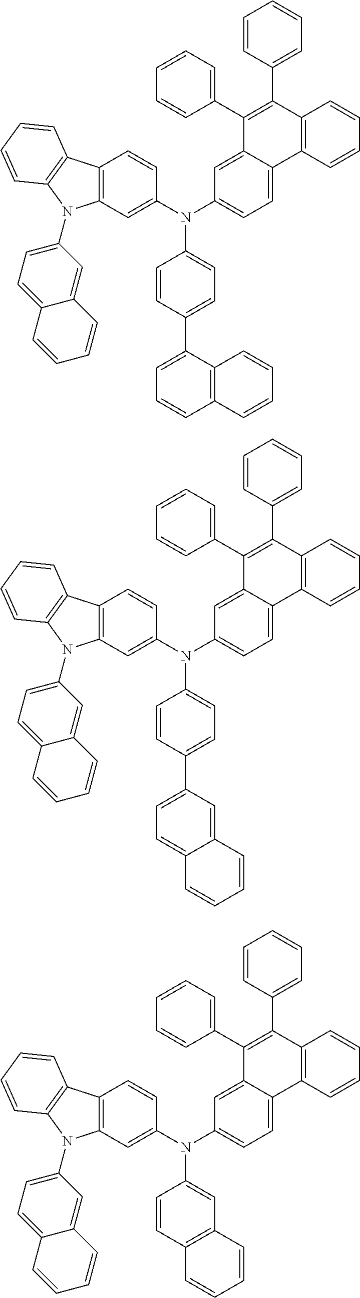

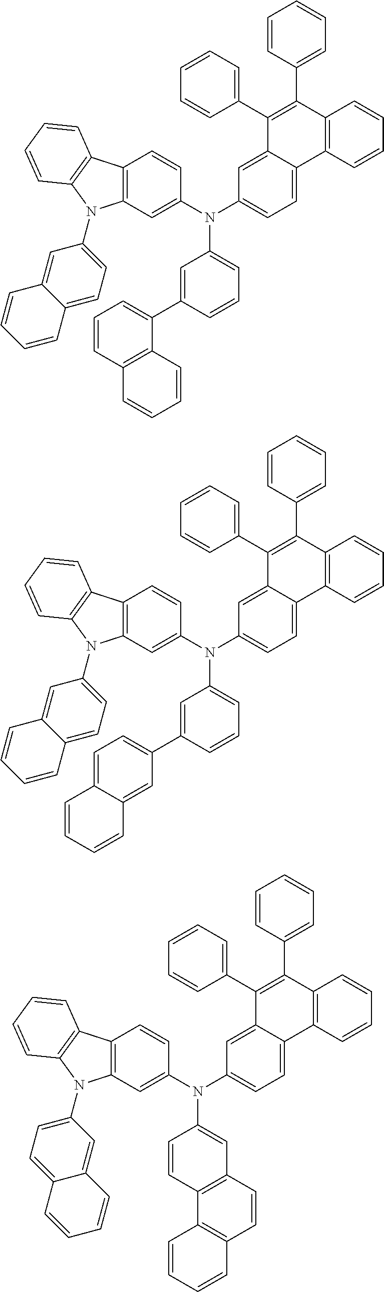 [SMILES: c1ccc(-c2c(-c3ccccc3)c3cc(N(c4cccc(-c5cccc6ccccc56)c4)c4ccc5c6ccccc6n(-c6ccc7ccccc7c6)c5c4)ccc3c3ccccc23)cc1 ; c1ccc(-c2c(-c3ccccc3)c3cc(N(c4cccc(-c5ccc6ccccc6c5)c4)c4ccc5c6ccccc6n(-c6ccc7ccccc7c6)c5c4)ccc3c3ccccc23)cc1 ; c1ccc(-c2c(-c3ccccc3)c3cc(N(c4ccc5c(ccc6ccccc65)c4)c4ccc5c6ccccc6n(-c6ccc7ccccc7c6)c5c4)ccc3c3ccccc23)cc1]

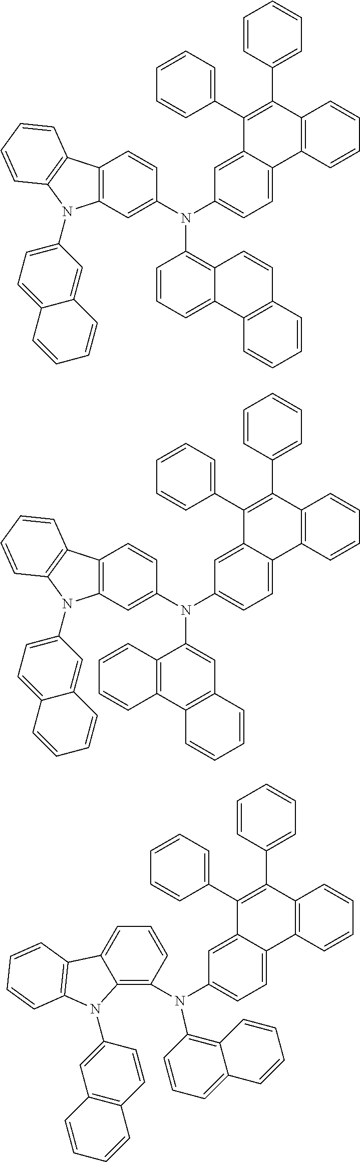

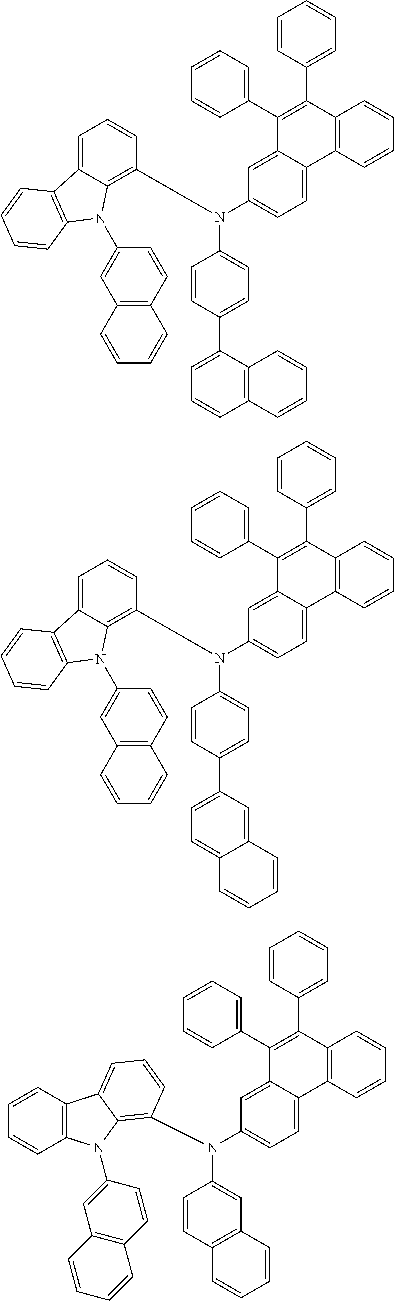

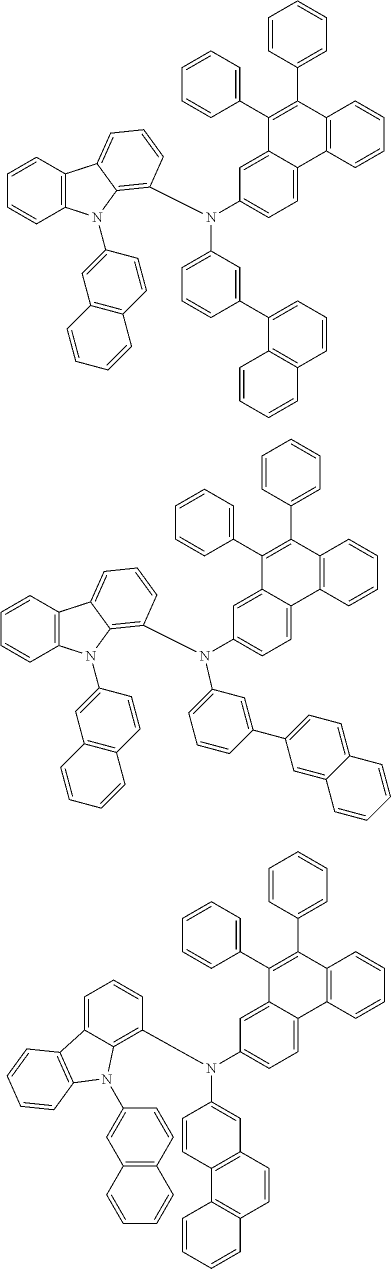

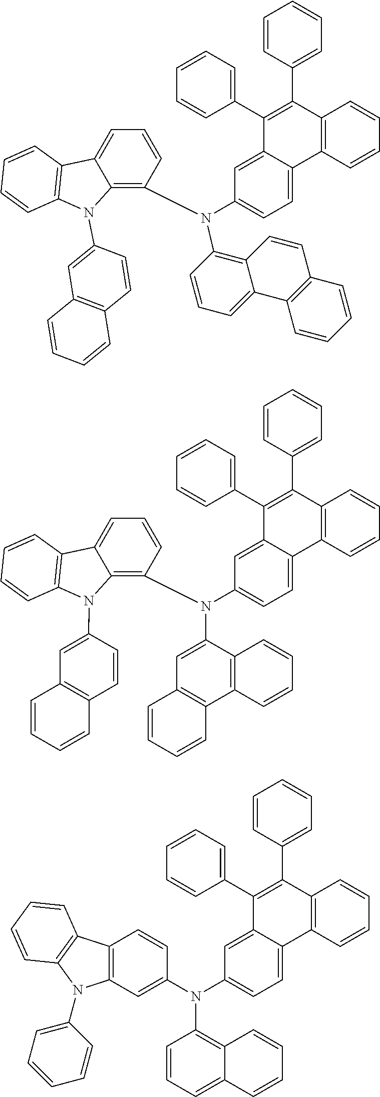 [SMILES: c1ccc(-c2c(-c3ccccc3)c3cc(N(c4cccc5c4ccc4ccccc45)c4cccc5c6ccccc6n(-c6ccc7ccccc7c6)c45)ccc3c3ccccc23)cc1 ; c1ccc(-c2c(-c3ccccc3)c3cc(N(c4cc5ccccc5c5ccccc45)c4cccc5c6ccccc6n(-c6ccc7ccccc7c6)c45)ccc3c3ccccc23)cc1 ; c1ccc(-c2c(-c3ccccc3)c3cc(N(c4ccc5c6ccccc6n(-c6ccccc6)c5c4)c4cccc5ccccc45)ccc3c3ccccc23)cc1]

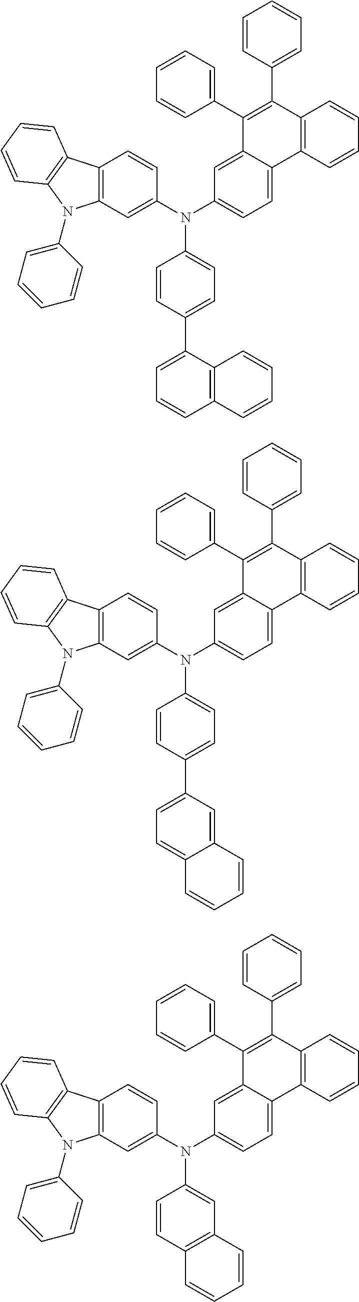

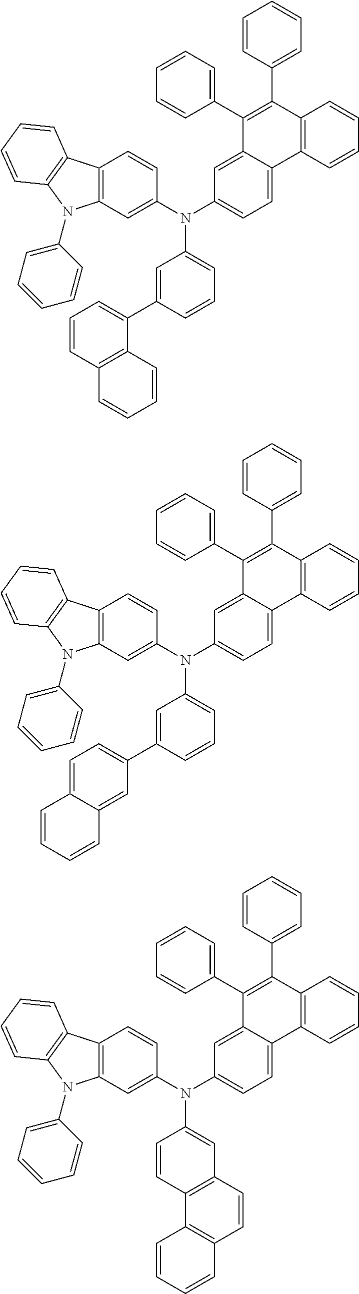 [SMILES: c1ccc(-c2c(-c3ccccc3)c3cc(N(c4cccc(-c5cccc6ccccc56)c4)c4ccc5c6ccccc6n(-c6ccccc6)c5c4)ccc3c3ccccc23)cc1 ; c1ccc(-c2c(-c3ccccc3)c3cc(N(c4cccc(-c5ccc6ccccc6c5)c4)c4ccc5c6ccccc6n(-c6ccccc6)c5c4)ccc3c3ccccc23)cc1 ; c1ccc(-c2c(-c3ccccc3)c3cc(N(c4ccc5c(ccc6ccccc65)c4)c4ccc5c6ccccc6n(-c6ccccc6)c5c4)ccc3c3ccccc23)cc1]

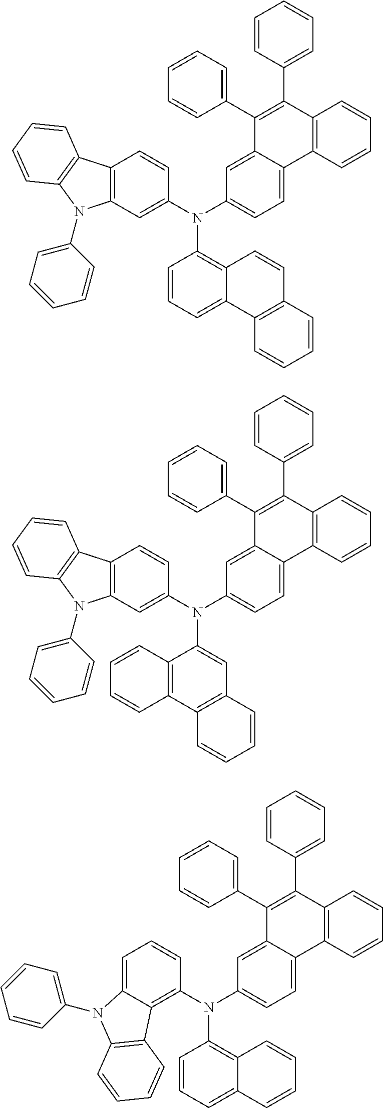

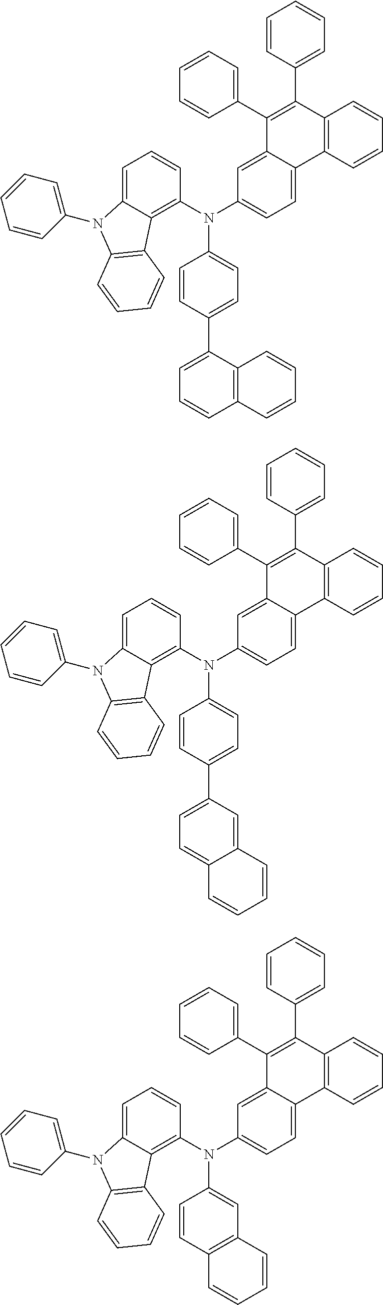

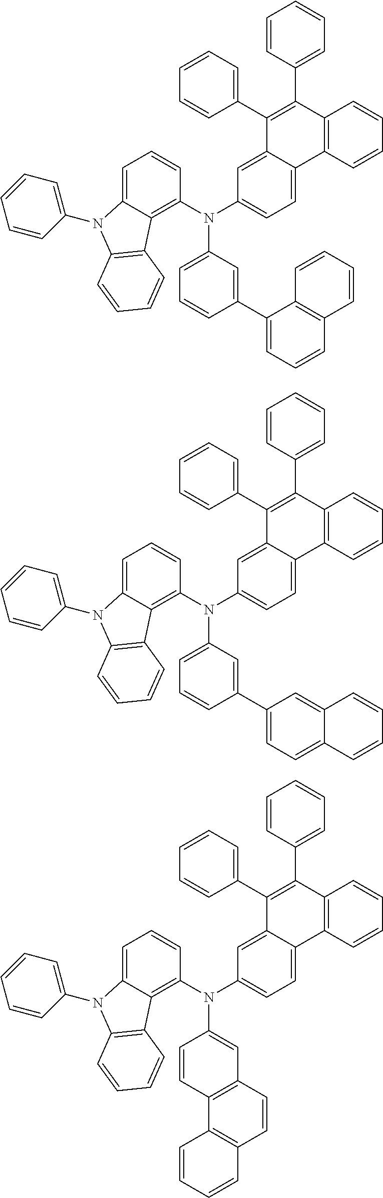

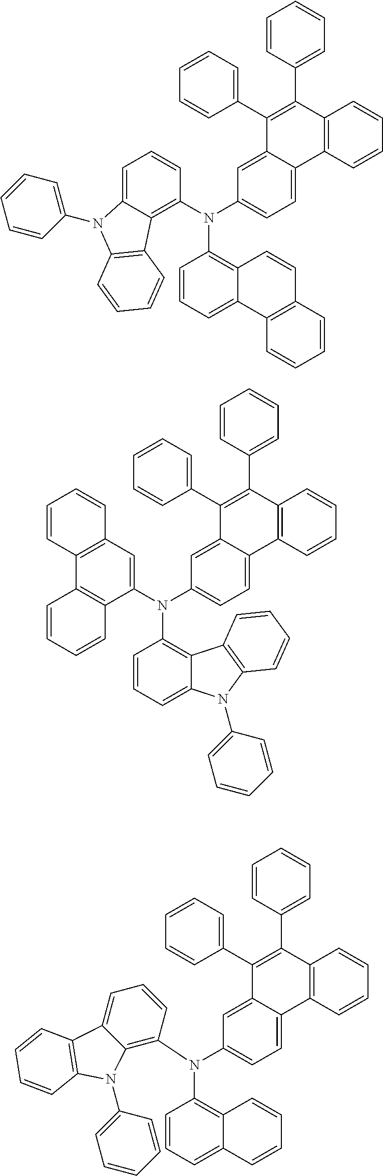

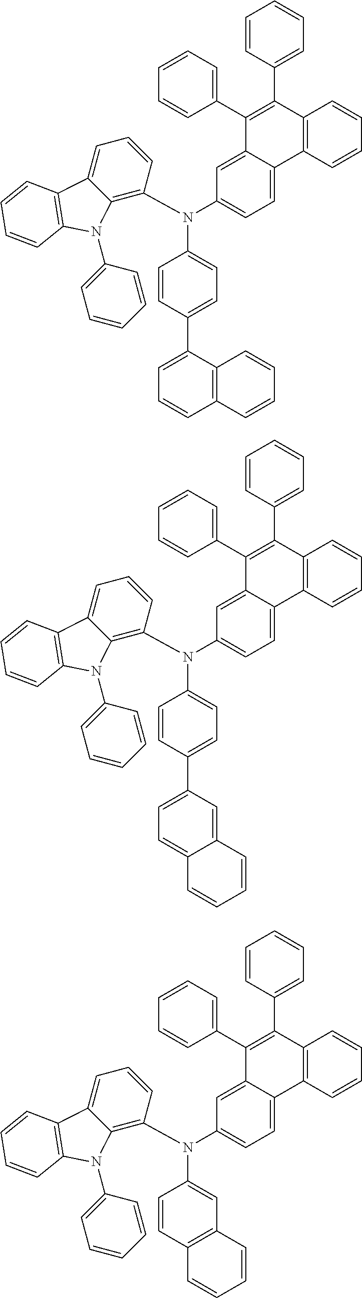

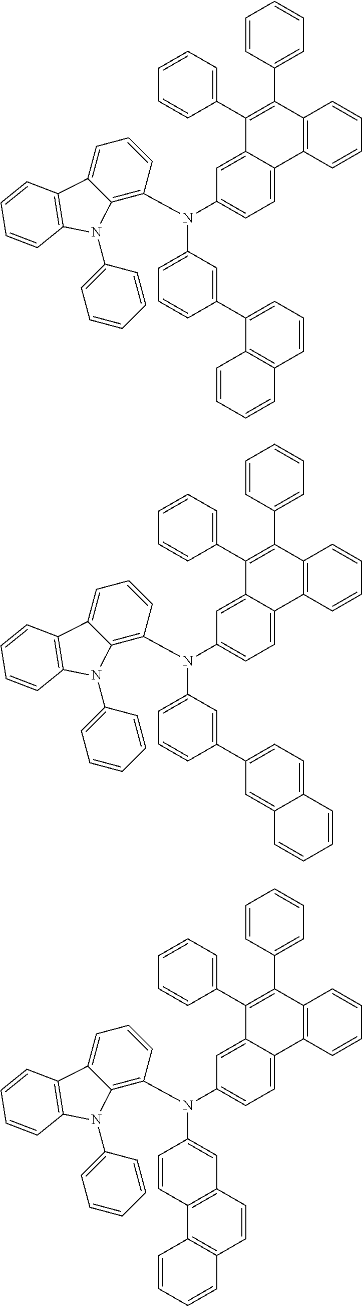

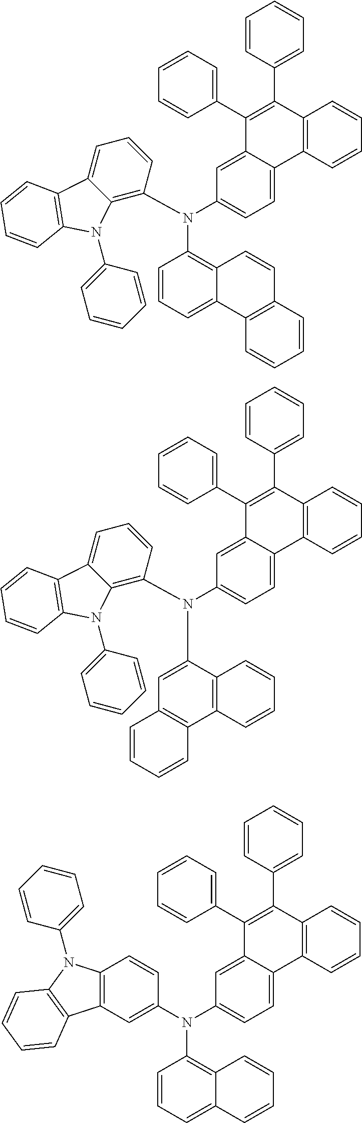

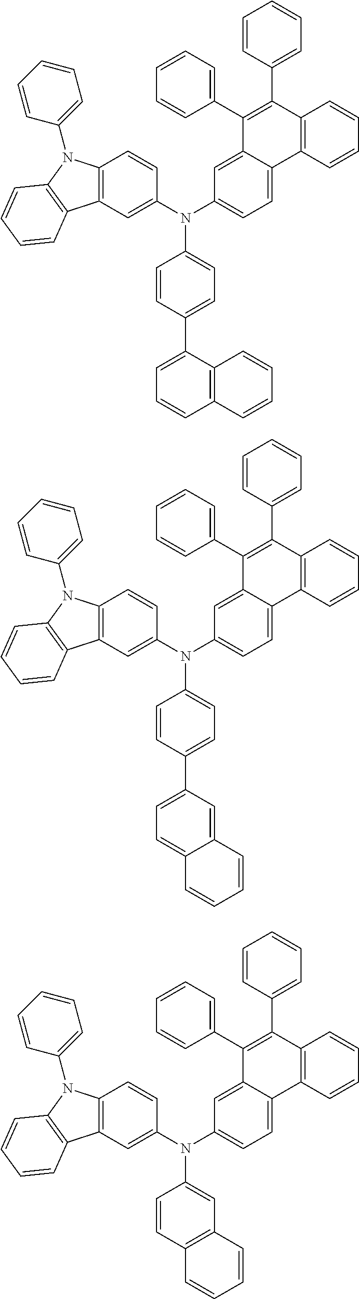

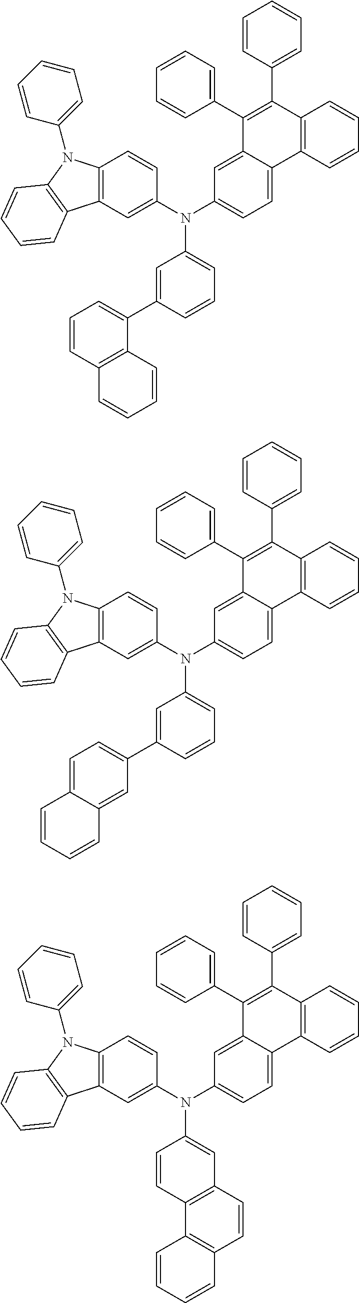 [SMILES: c1ccc(-c2c(-c3ccccc3)c3cc(N(c4cccc(-c5cccc6ccccc56)c4)c4ccc5c(c4)c4ccccc4n5-c4ccccc4)ccc3c3ccccc23)cc1 ; c1ccc(-c2c(-c3ccccc3)c3cc(N(c4cccc(-c5ccc6ccccc6c5)c4)c4ccc5c(c4)c4ccccc4n5-c4ccccc4)ccc3c3ccccc23)cc1 ; c1ccc(-c2c(-c3ccccc3)c3cc(N(c4ccc5c(ccc6ccccc65)c4)c4ccc5c(c4)c4ccccc4n5-c4ccccc4)ccc3c3ccccc23)cc1]

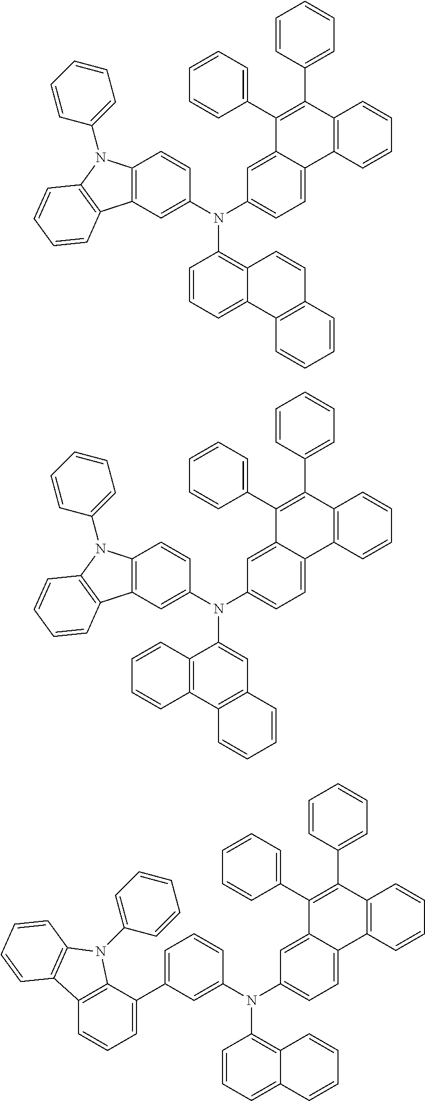

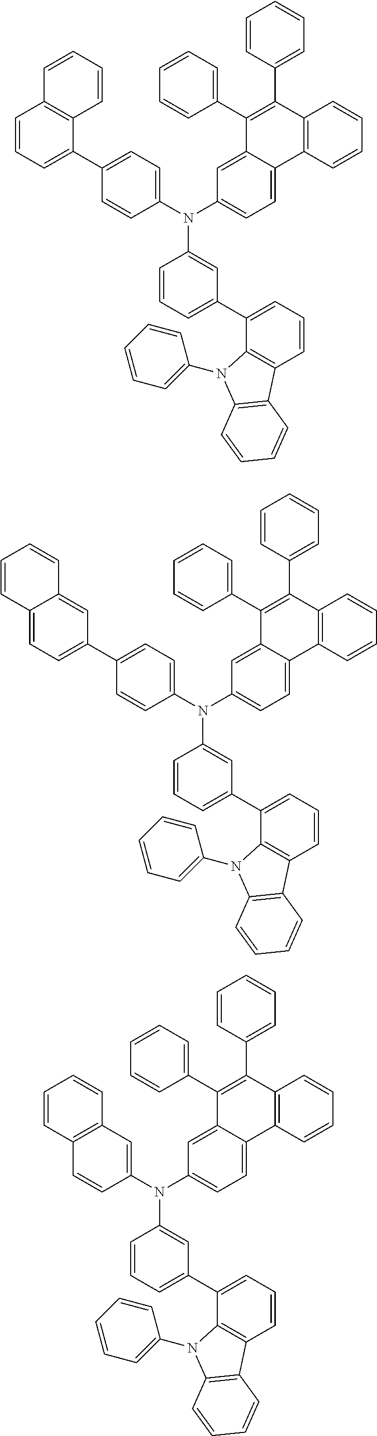

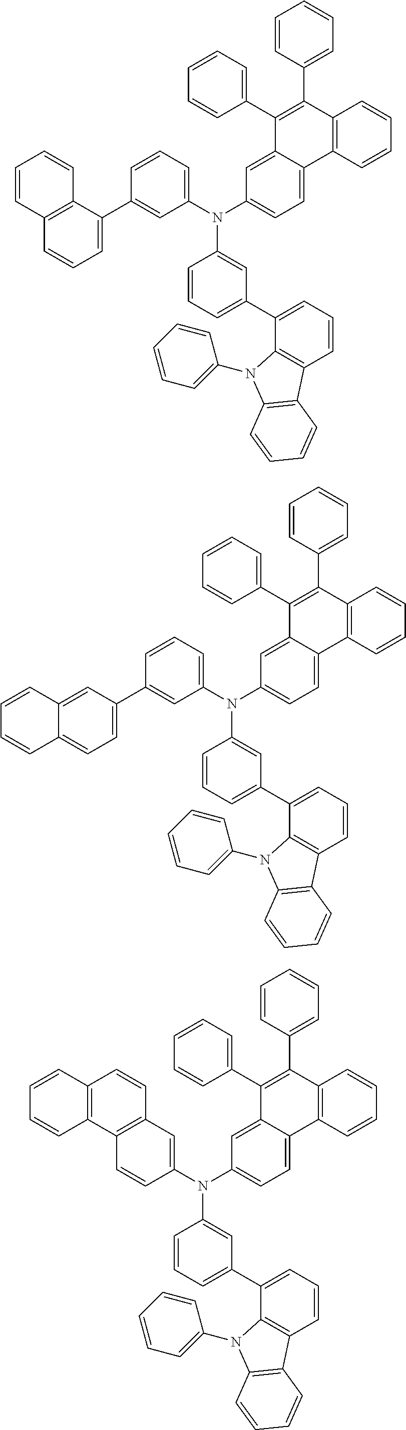

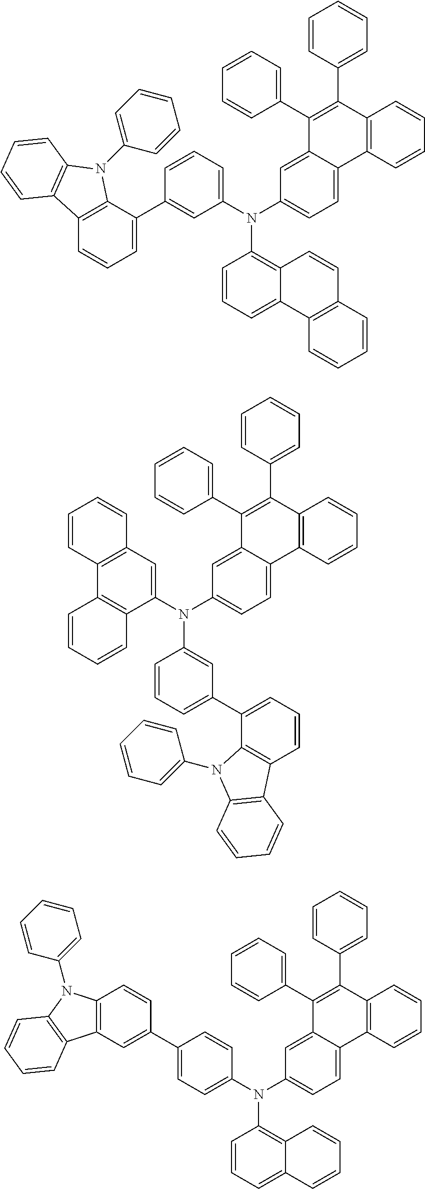 [SMILES: c1ccc(-c2c(-c3ccccc3)c3cc(N(c4cccc(-c5cccc6c7ccccc7n(-c7ccccc7)c56)c4)c4cccc5c4ccc4ccccc45)ccc3c3ccccc23)cc1 ; c1ccc(-c2c(-c3ccccc3)c3cc(N(c4cccc(-c5cccc6c7ccccc7n(-c7ccccc7)c56)c4)c4cc5ccccc5c5ccccc45)ccc3c3ccccc23)cc1 ; c1ccc(-c2c(-c3ccccc3)c3cc(N(c4ccc(-c5ccc6c(c5)c5ccccc5n6-c5ccccc5)cc4)c4cccc5ccccc45)ccc3c3ccccc23)cc1]

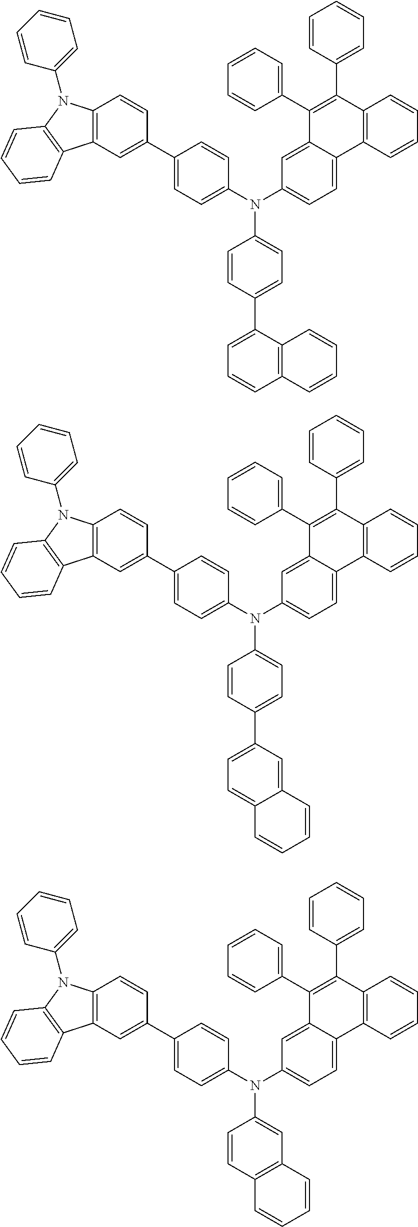

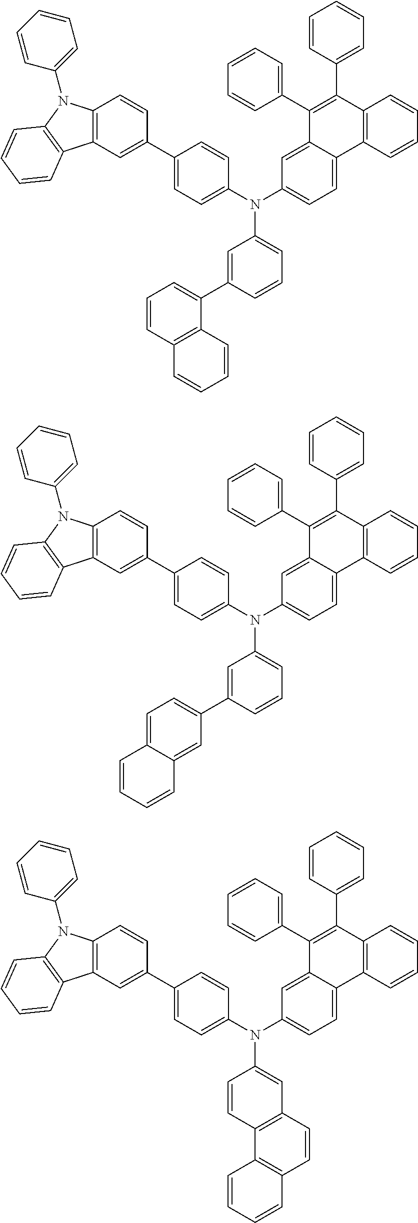 [SMILES: c1ccc(-c2c(-c3ccccc3)c3cc(N(c4ccc(-c5ccc6c(c5)c5ccccc5n6-c5ccccc5)cc4)c4cccc(-c5cccc6ccccc56)c4)ccc3c3ccccc23)cc1 ; c1ccc(-c2c(-c3ccccc3)c3cc(N(c4ccc(-c5ccc6c(c5)c5ccccc5n6-c5ccccc5)cc4)c4cccc(-c5ccc6ccccc6c5)c4)ccc3c3ccccc23)cc1 ; c1ccc(-c2c(-c3ccccc3)c3cc(N(c4ccc(-c5ccc6c(c5)c5ccccc5n6-c5ccccc5)cc4)c4ccc5c(ccc6ccccc65)c4)ccc3c3ccccc23)cc1]

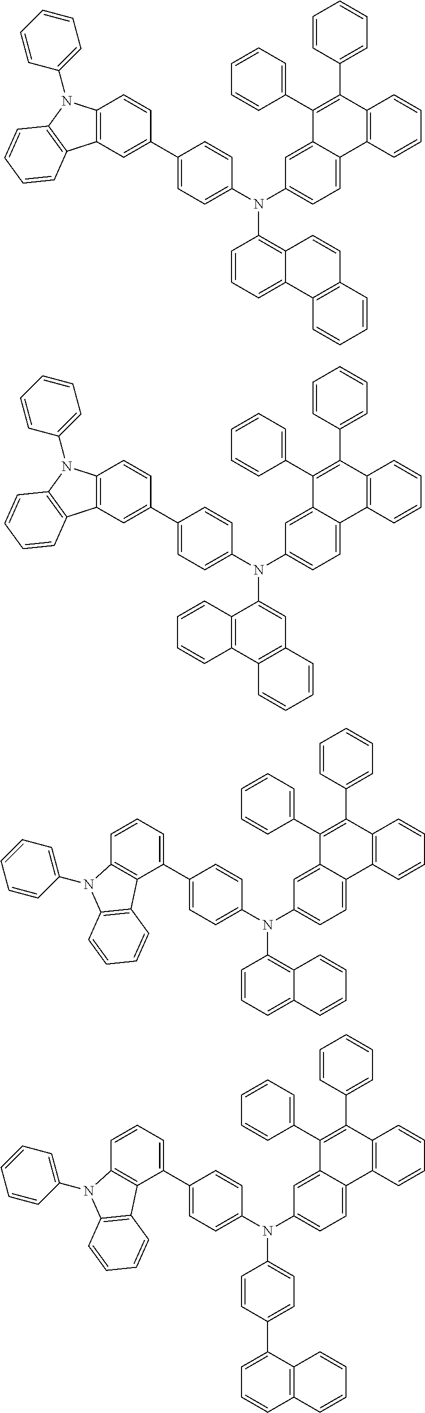

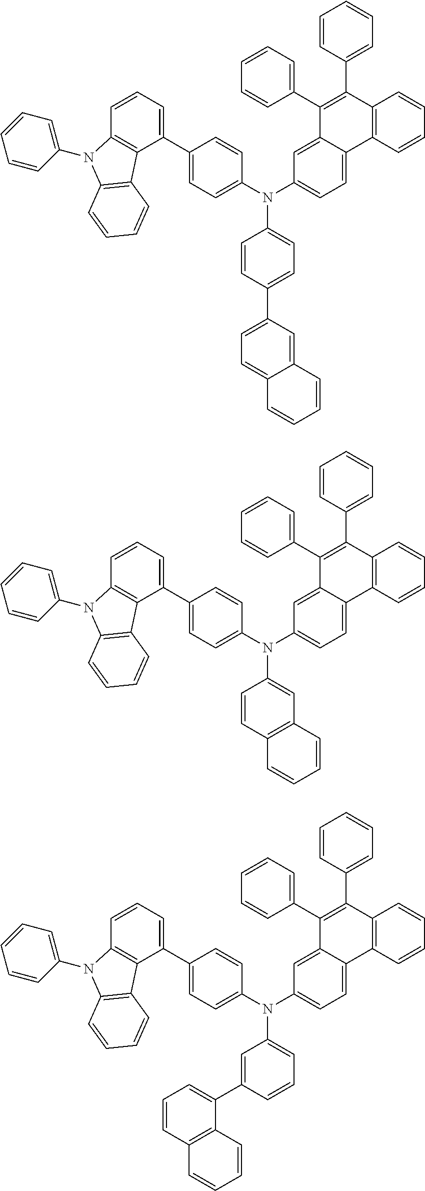 [SMILES: c1ccc(-c2c(-c3ccccc3)c3cc(N(c4ccc(-c5ccc6ccccc6c5)cc4)c4ccc(-c5cccc6c5c5ccccc5n6-c5ccccc5)cc4)ccc3c3ccccc23)cc1 ; c1ccc(-c2c(-c3ccccc3)c3cc(N(c4ccc(-c5cccc6c5c5ccccc5n6-c5ccccc5)cc4)c4ccc5ccccc5c4)ccc3c3ccccc23)cc1 ; c1ccc(-c2c(-c3ccccc3)c3cc(N(c4ccc(-c5cccc6c5c5ccccc5n6-c5ccccc5)cc4)c4cccc(-c5cccc6ccccc56)c4)ccc3c3ccccc23)cc1]

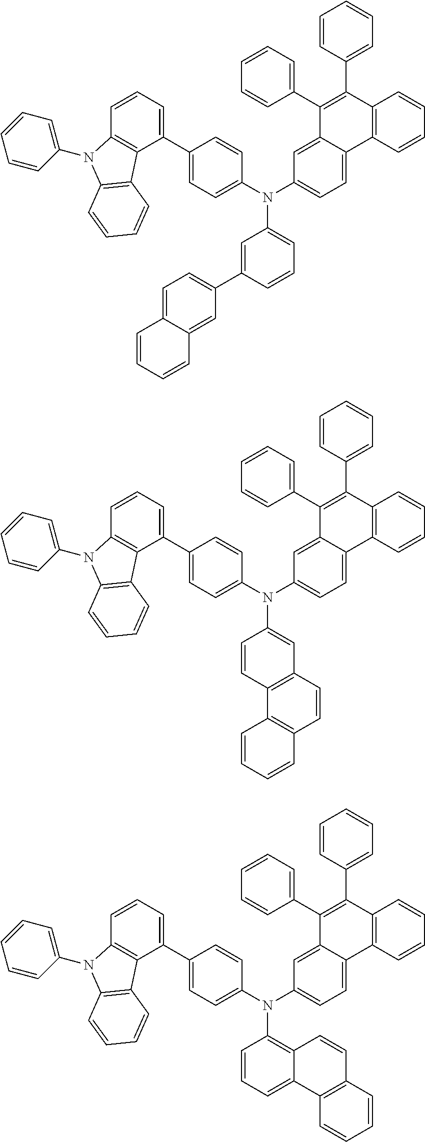 [SMILES: c1ccc(-c2c(-c3ccccc3)c3cc(N(c4ccc(-c5cccc6c5c5ccccc5n6-c5ccccc5)cc4)c4cccc(-c5ccc6ccccc6c5)c4)ccc3c3ccccc23)cc1 ; c1ccc(-c2c(-c3ccccc3)c3cc(N(c4ccc(-c5cccc6c5c5ccccc5n6-c5ccccc5)cc4)c4ccc5c(ccc6ccccc65)c4)ccc3c3ccccc23)cc1 ; c1ccc(-c2c(-c3ccccc3)c3cc(N(c4ccc(-c5cccc6c5c5ccccc5n6-c5ccccc5)cc4)c4cccc5c4ccc4ccccc45)ccc3c3ccccc23)cc1]

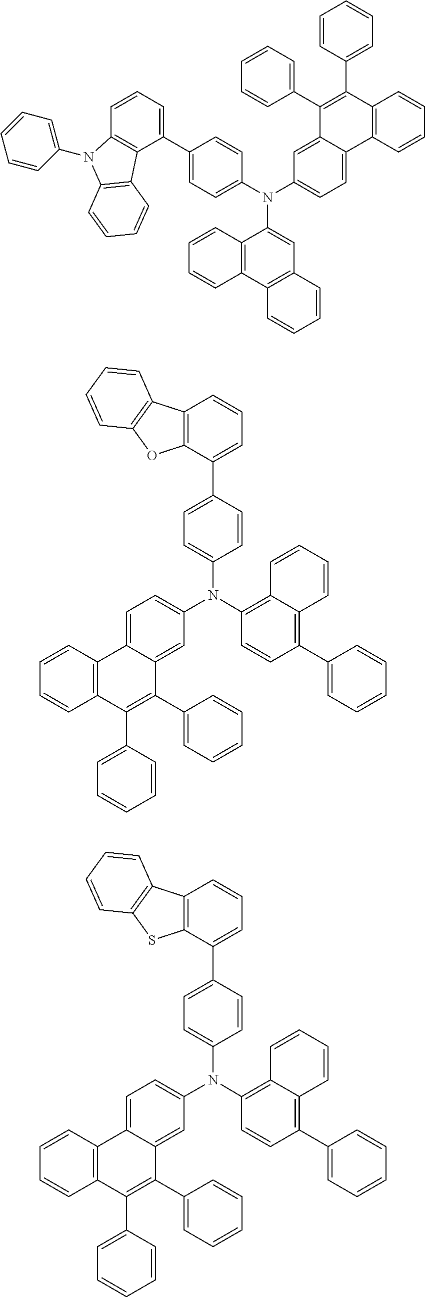

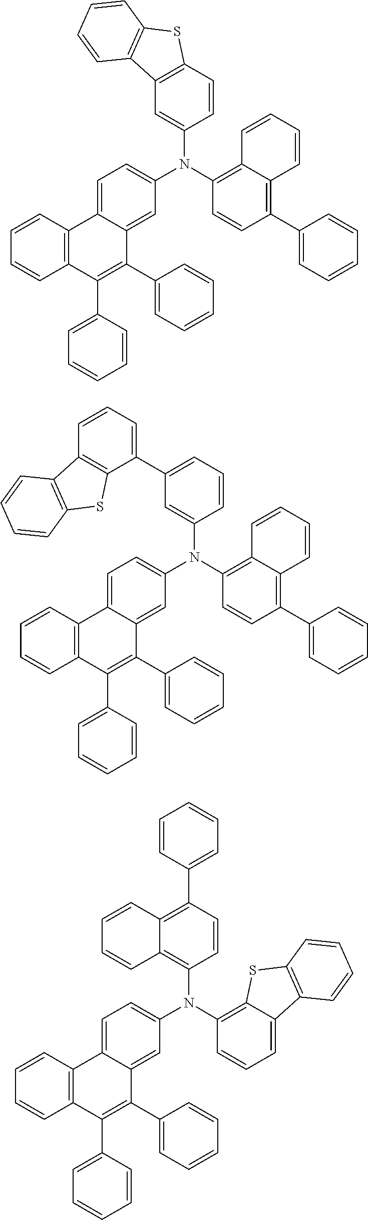

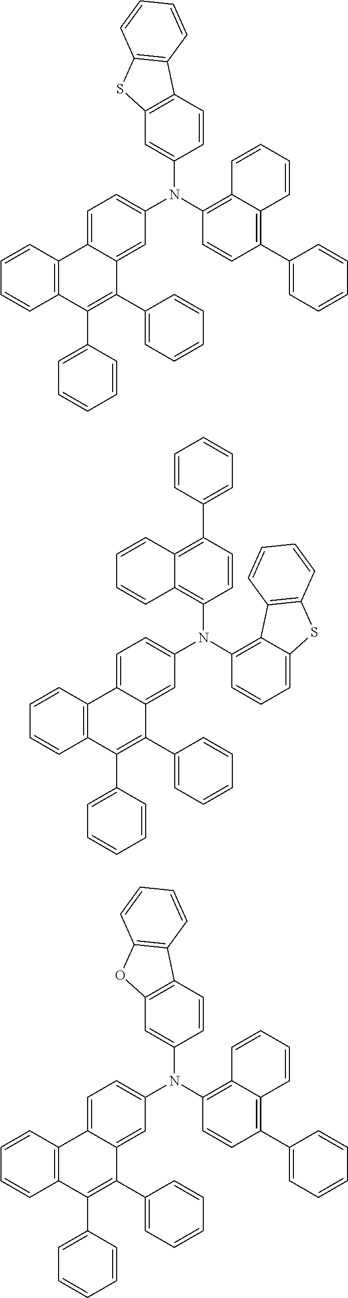

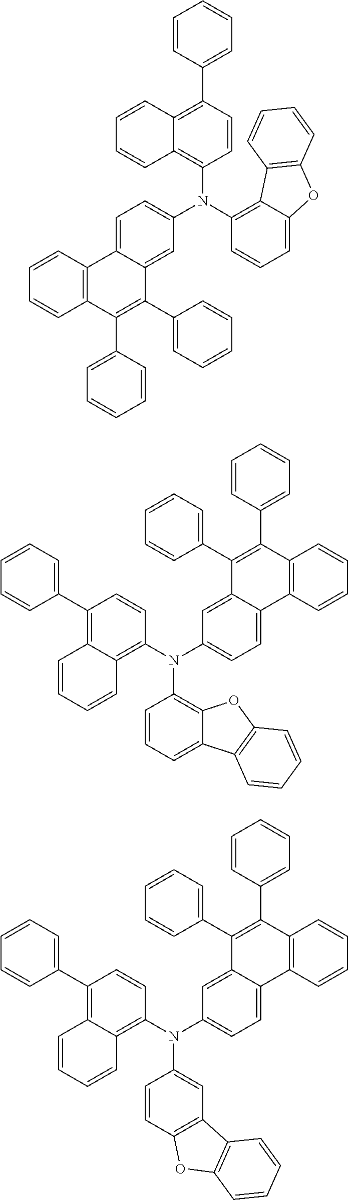

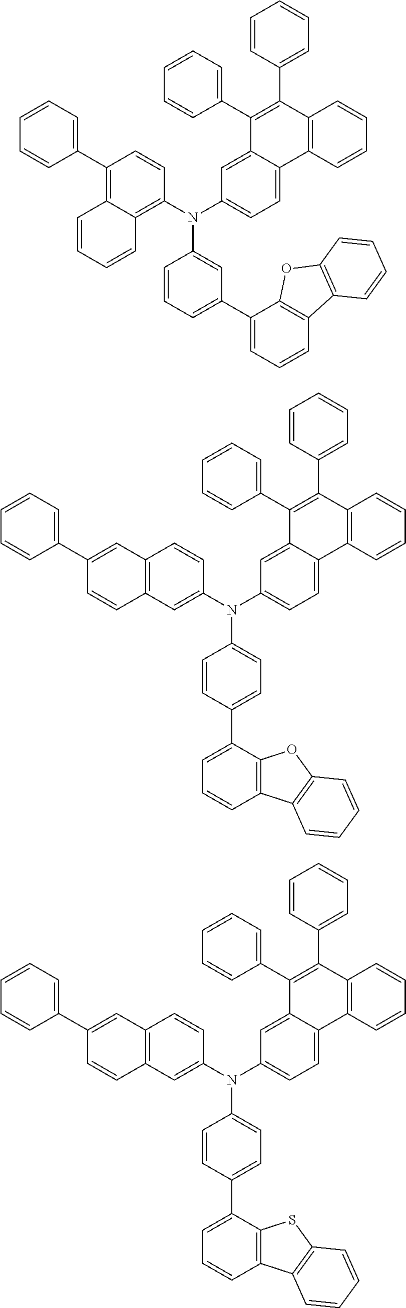

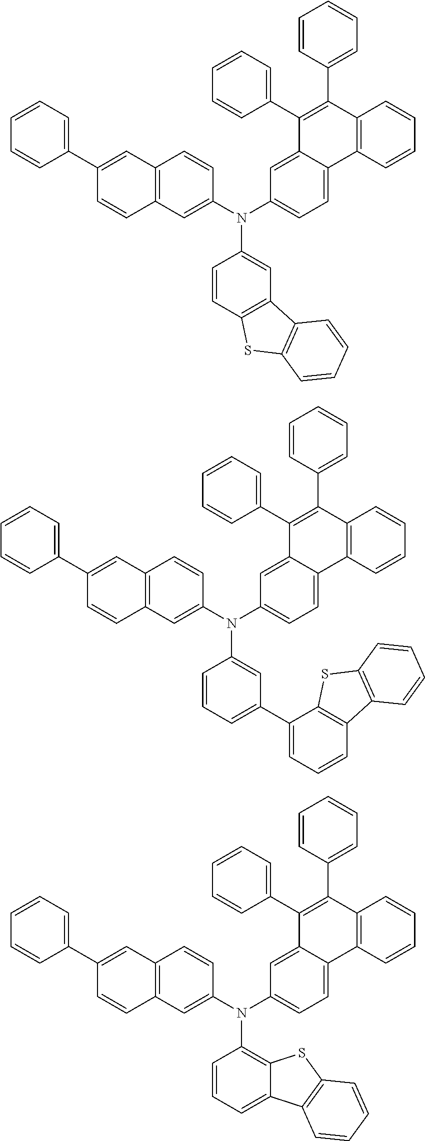

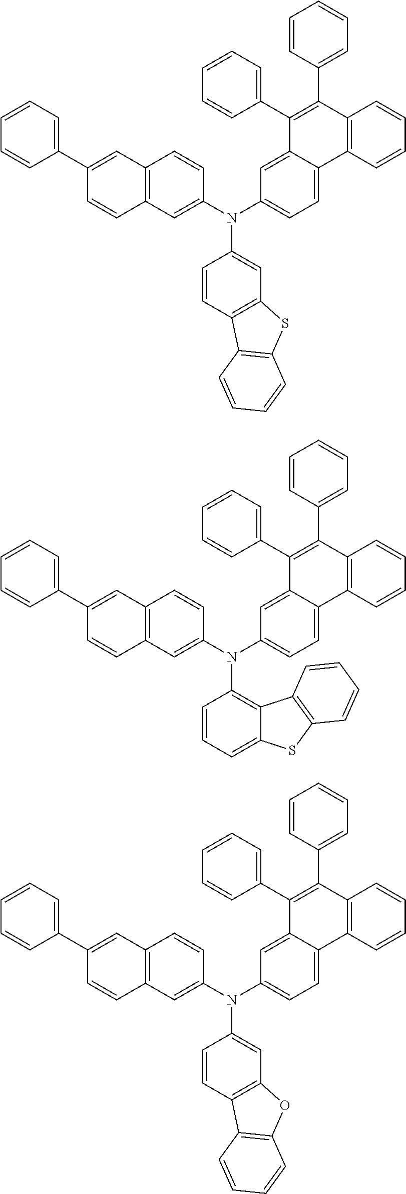

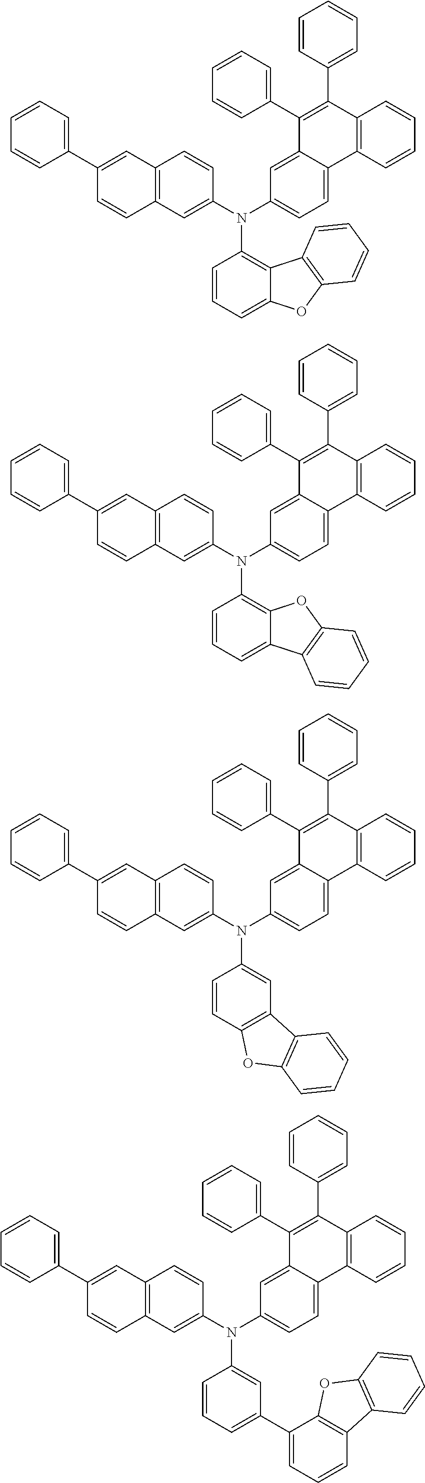

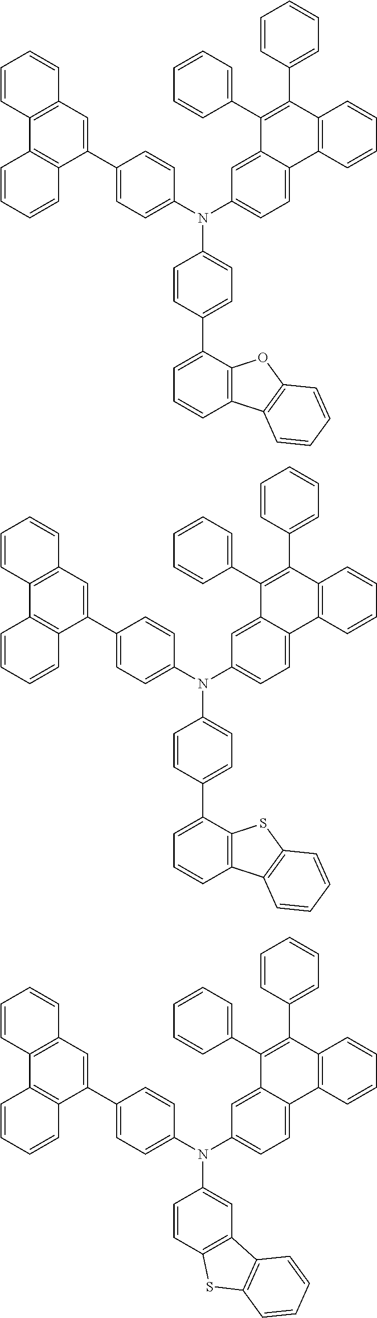

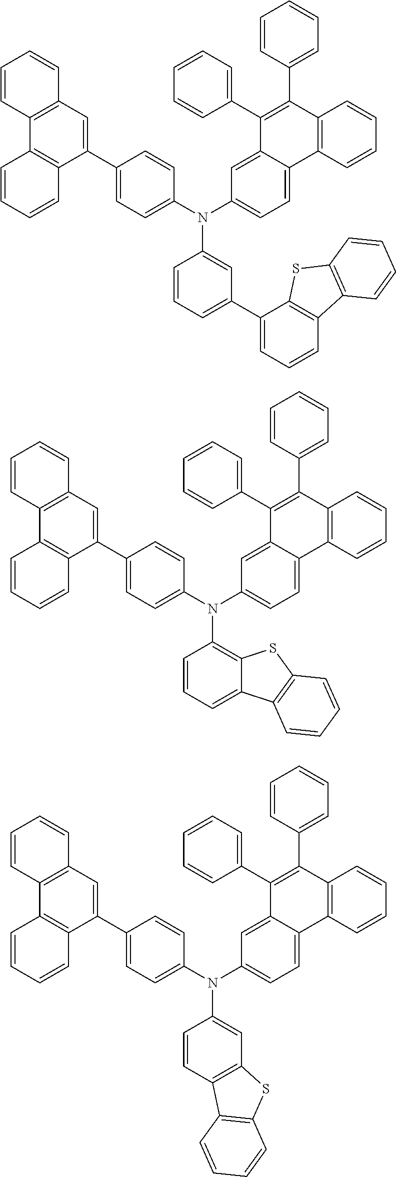

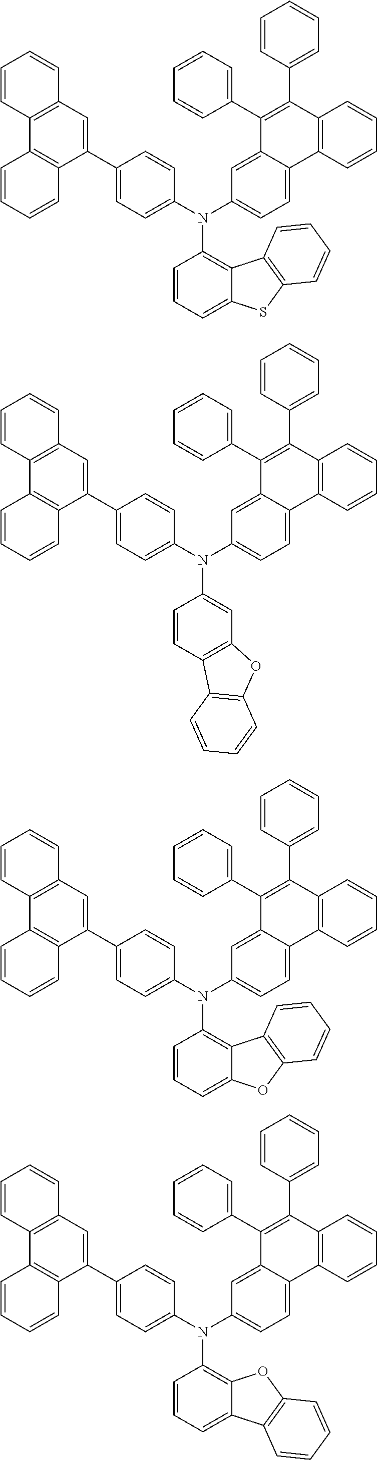

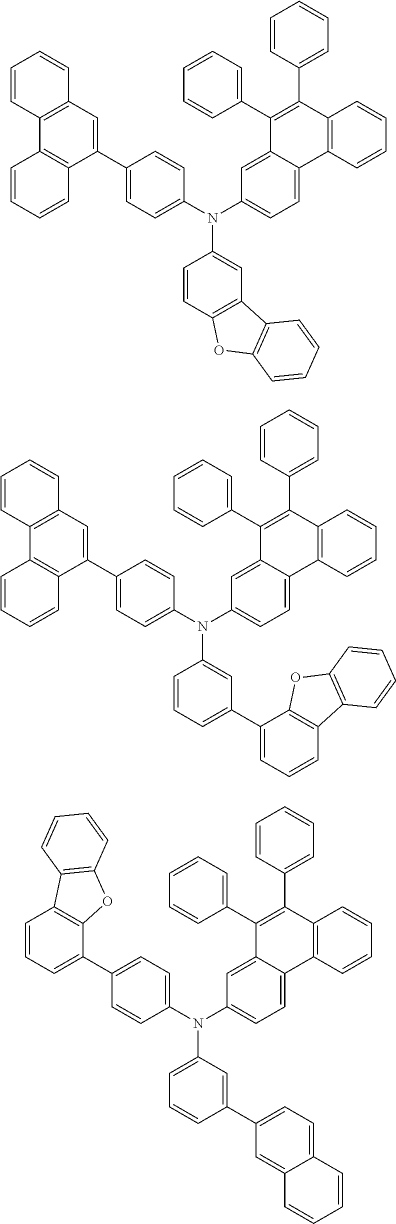

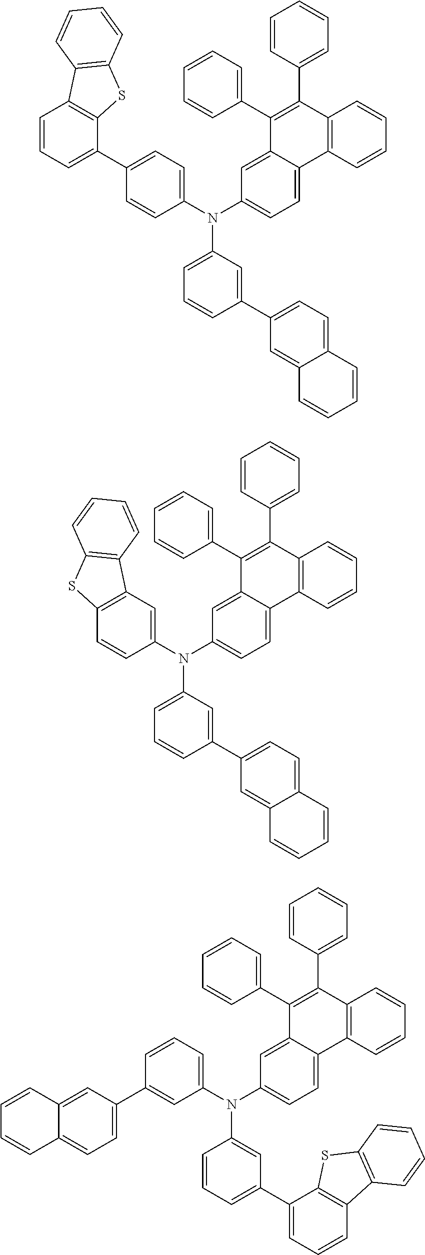

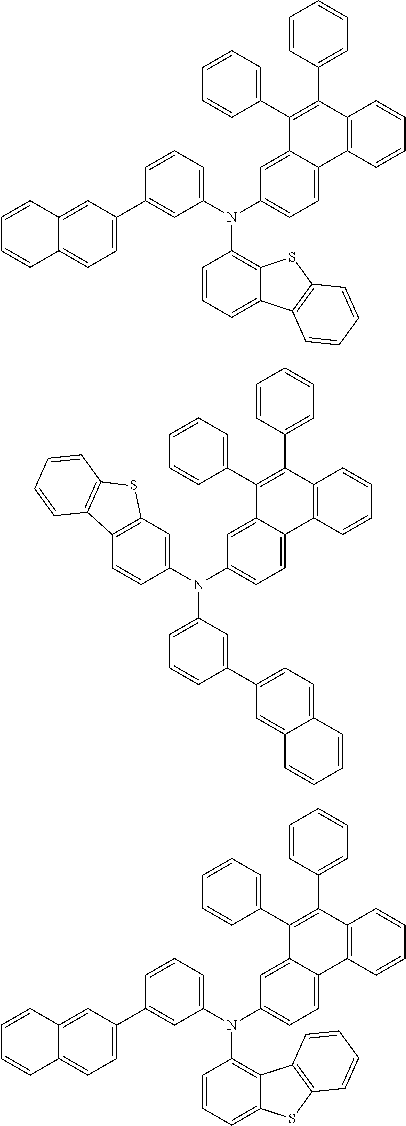

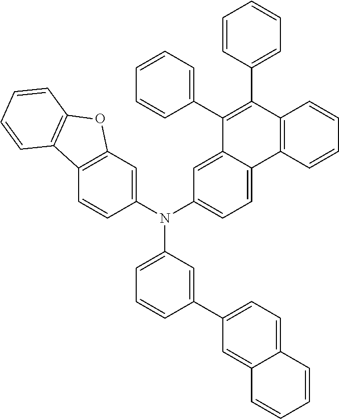 [SMILES: c1ccc(-c2c(-c3ccccc3)c3cc(N(c4cccc(-c5ccc6ccccc6c5)c4)c4ccc5c(c4)oc4ccccc45)ccc3c3ccccc23)cc1]

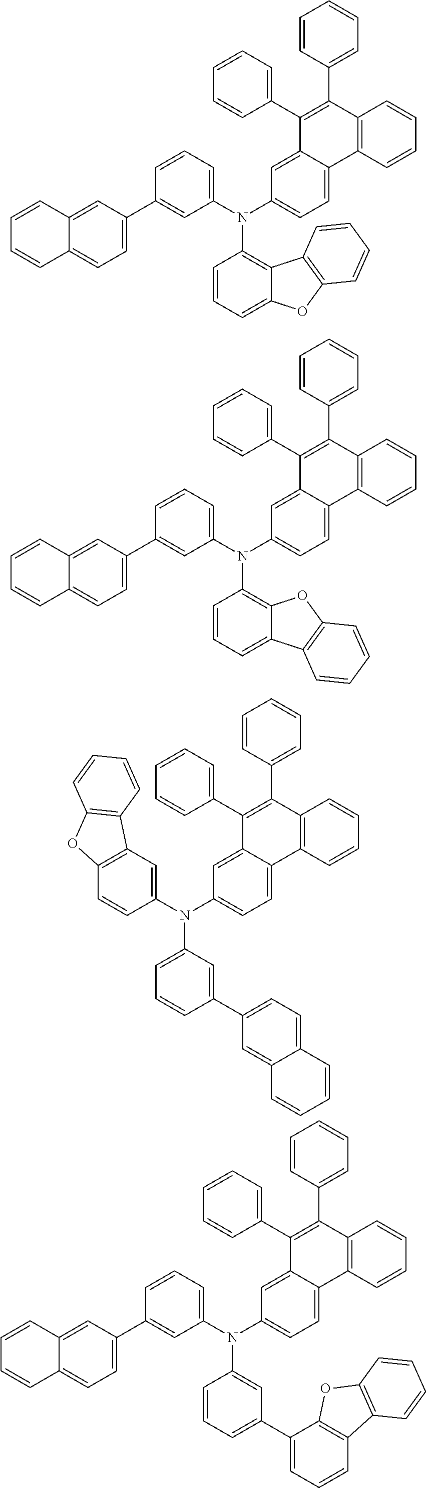

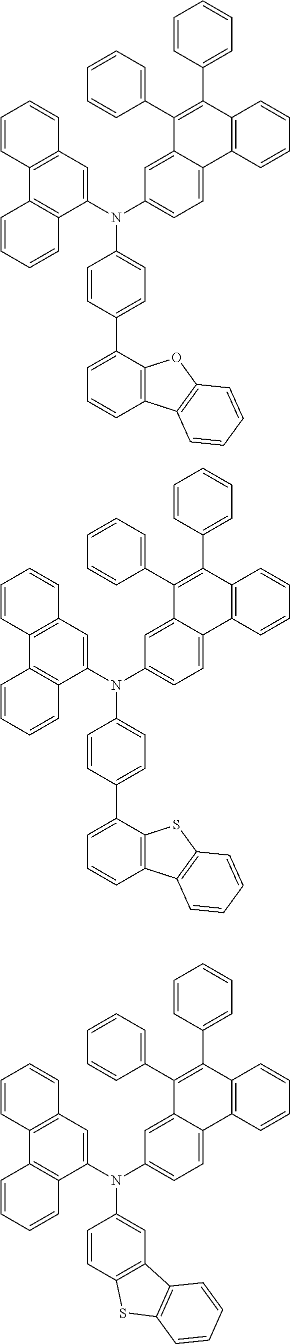

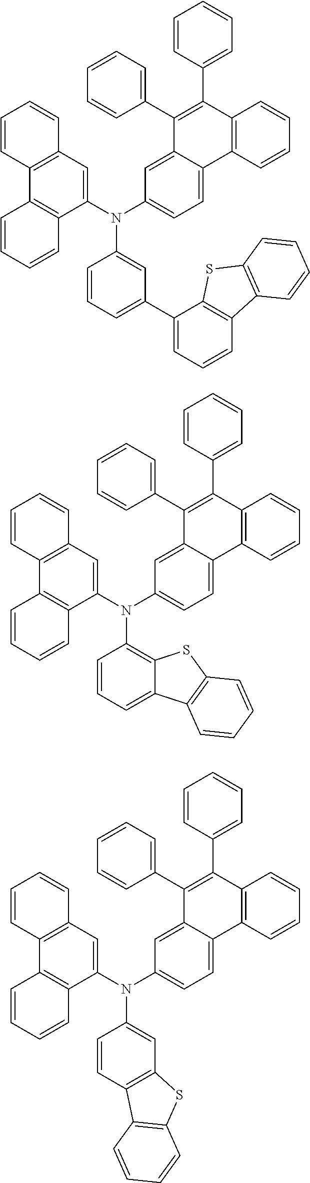

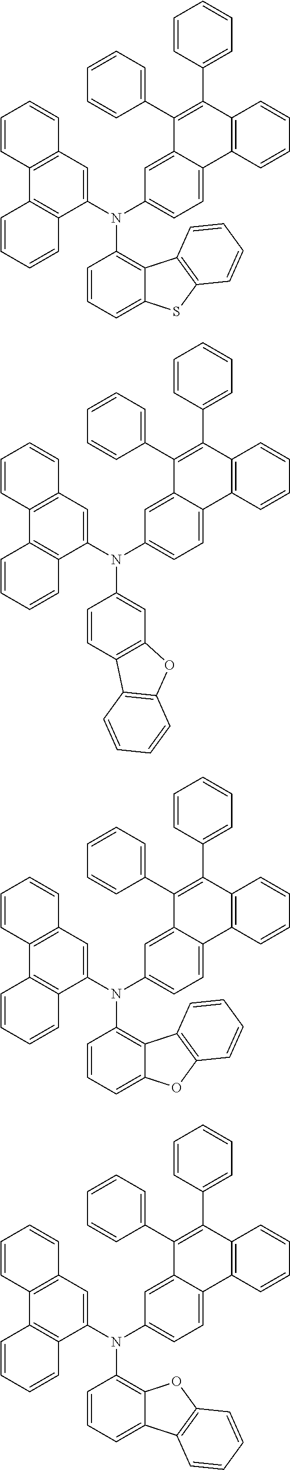

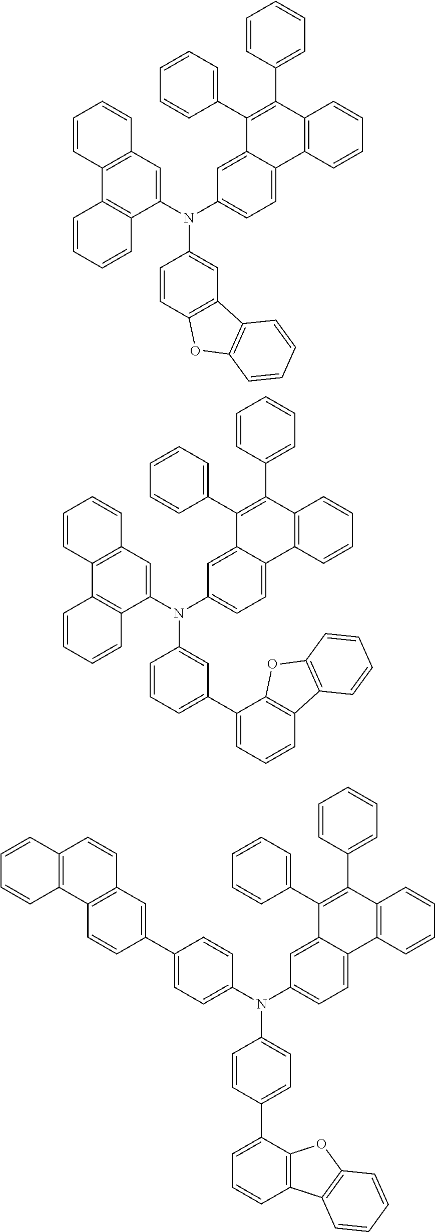

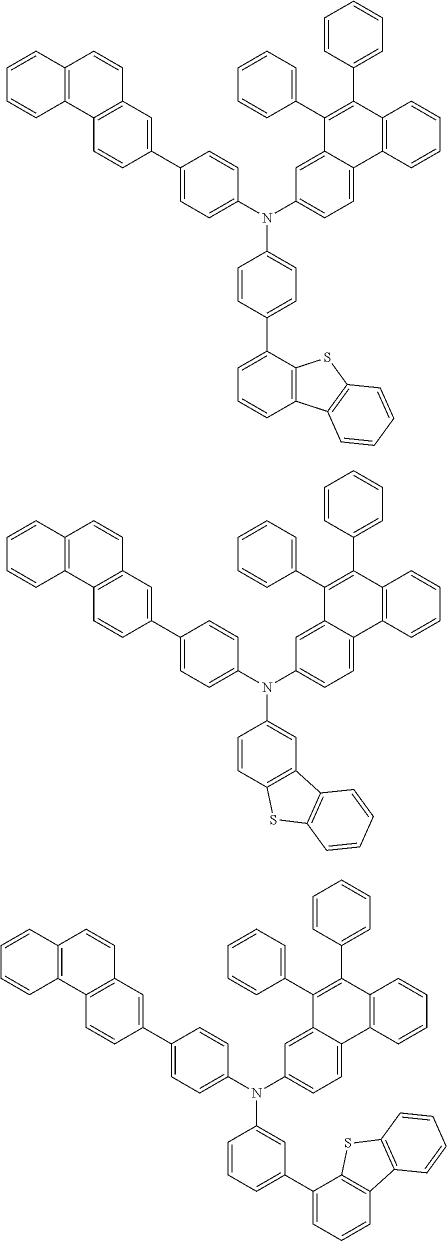

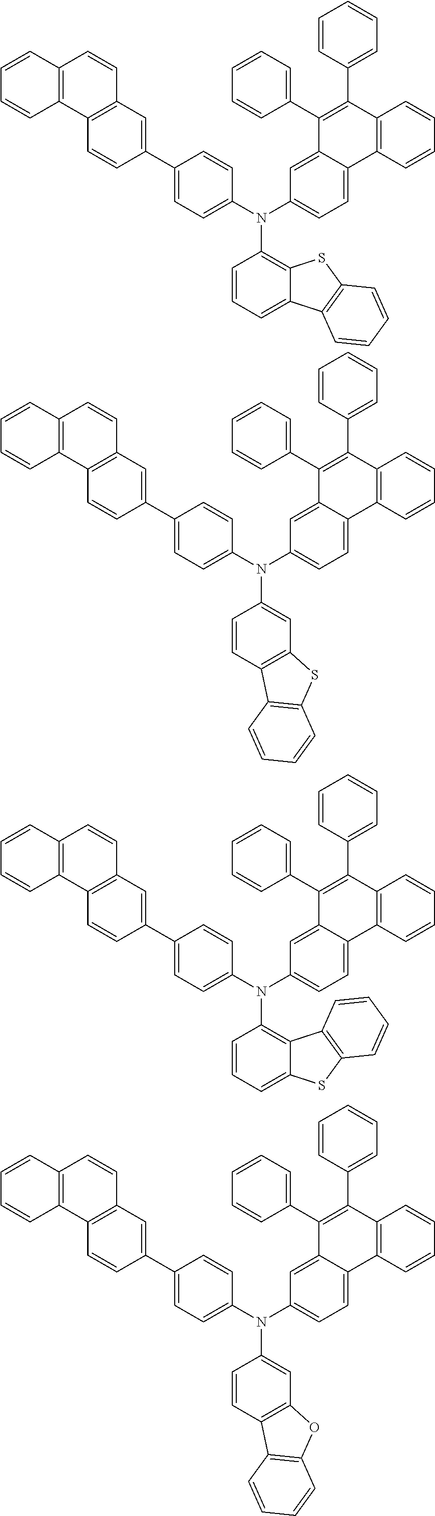

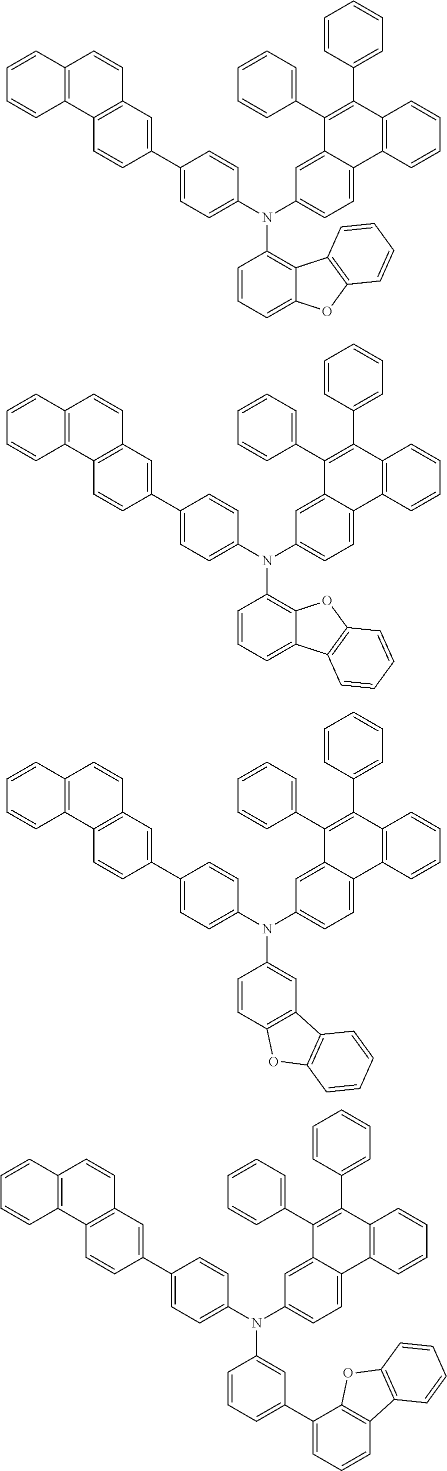

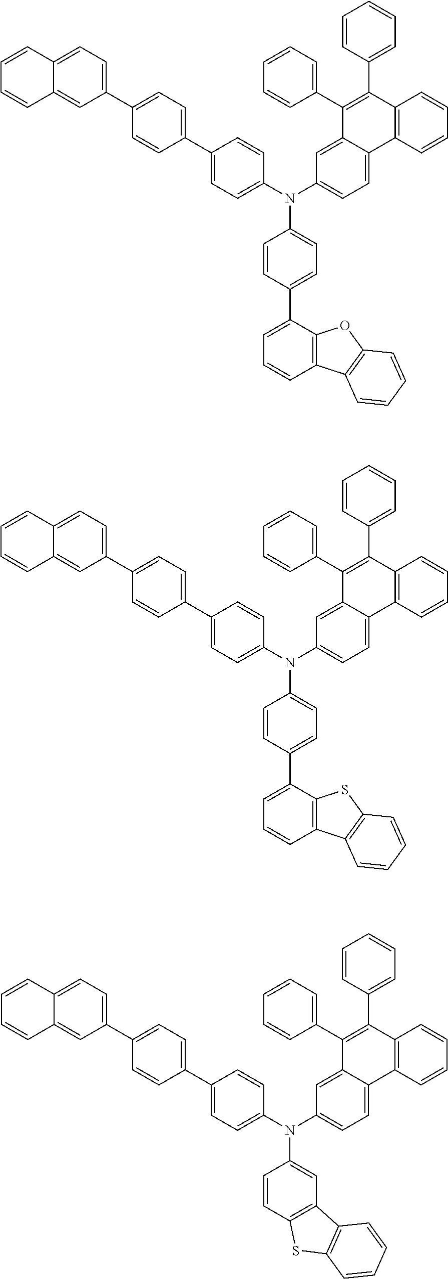 [SMILES: c1ccc(-c2c(-c3ccccc3)c3cc(N(c4ccc(-c5ccc(-c6ccc7ccccc7c6)cc5)cc4)c4ccc(-c5cccc6c5oc5ccccc56)cc4)ccc3c3ccccc23)cc1 ; c1ccc(-c2c(-c3ccccc3)c3cc(N(c4ccc(-c5ccc(-c6ccc7ccccc7c6)cc5)cc4)c4ccc(-c5cccc6c5sc5ccccc56)cc4)ccc3c3ccccc23)cc1 ; c1ccc(-c2c(-c3ccccc3)c3cc(N(c4ccc(-c5ccc(-c6ccc7ccccc7c6)cc5)cc4)c4ccc5sc6ccccc6c5c4)ccc3c3ccccc23)cc1]

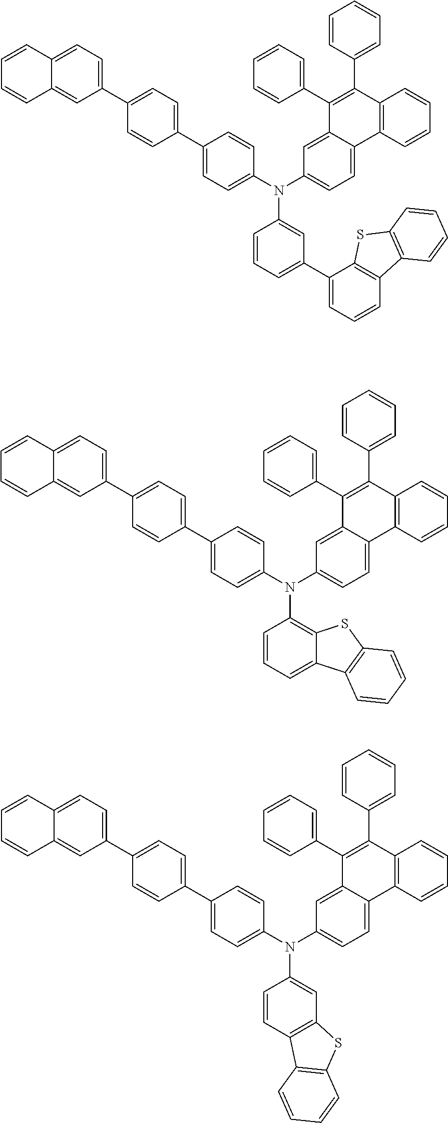 [SMILES: c1ccc(-c2c(-c3ccccc3)c3cc(N(c4ccc(-c5ccc(-c6ccc7ccccc7c6)cc5)cc4)c4cccc(-c5cccc6c5sc5ccccc56)c4)ccc3c3ccccc23)cc1 ; c1ccc(-c2c(-c3ccccc3)c3cc(N(c4ccc(-c5ccc(-c6ccc7ccccc7c6)cc5)cc4)c4cccc5c4sc4ccccc45)ccc3c3ccccc23)cc1 ; c1ccc(-c2c(-c3ccccc3)c3cc(N(c4ccc(-c5ccc(-c6ccc7ccccc7c6)cc5)cc4)c4ccc5c(c4)sc4ccccc45)ccc3c3ccccc23)cc1]

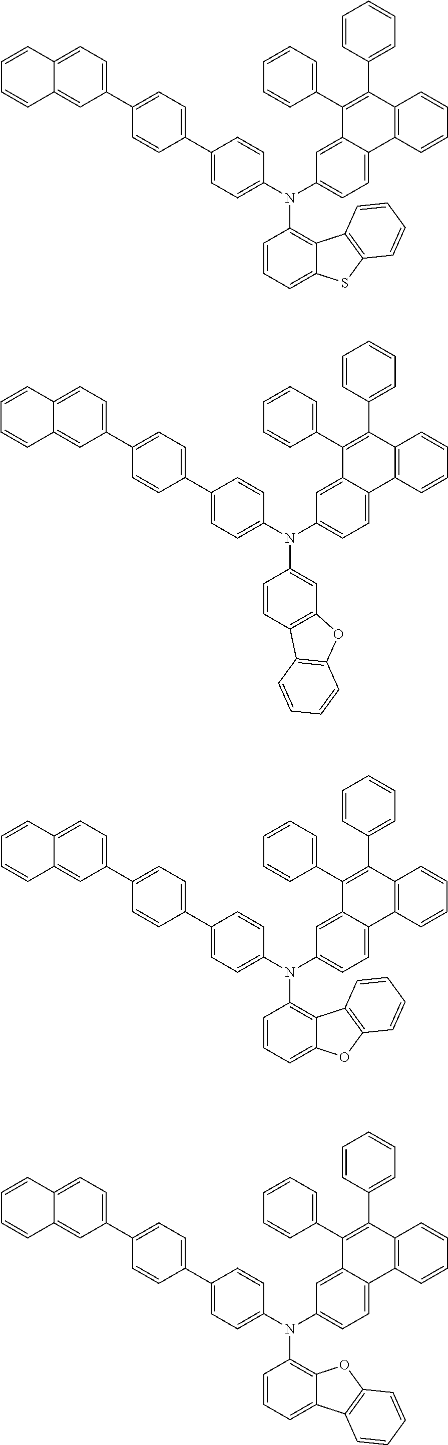 [SMILES: c1ccc(-c2c(-c3ccccc3)c3cc(N(c4ccc(-c5ccc(-c6ccc7ccccc7c6)cc5)cc4)c4cccc5sc6ccccc6c45)ccc3c3ccccc23)cc1 ; c1ccc(-c2c(-c3ccccc3)c3cc(N(c4ccc(-c5ccc(-c6ccc7ccccc7c6)cc5)cc4)c4ccc5c(c4)oc4ccccc45)ccc3c3ccccc23)cc1 ; c1ccc(-c2c(-c3ccccc3)c3cc(N(c4ccc(-c5ccc(-c6ccc7ccccc7c6)cc5)cc4)c4cccc5oc6ccccc6c45)ccc3c3ccccc23)cc1 ; c1ccc(-c2c(-c3ccccc3)c3cc(N(c4ccc(-c5ccc(-c6ccc7ccccc7c6)cc5)cc4)c4cccc5c4oc4ccccc45)ccc3c3ccccc23)cc1]

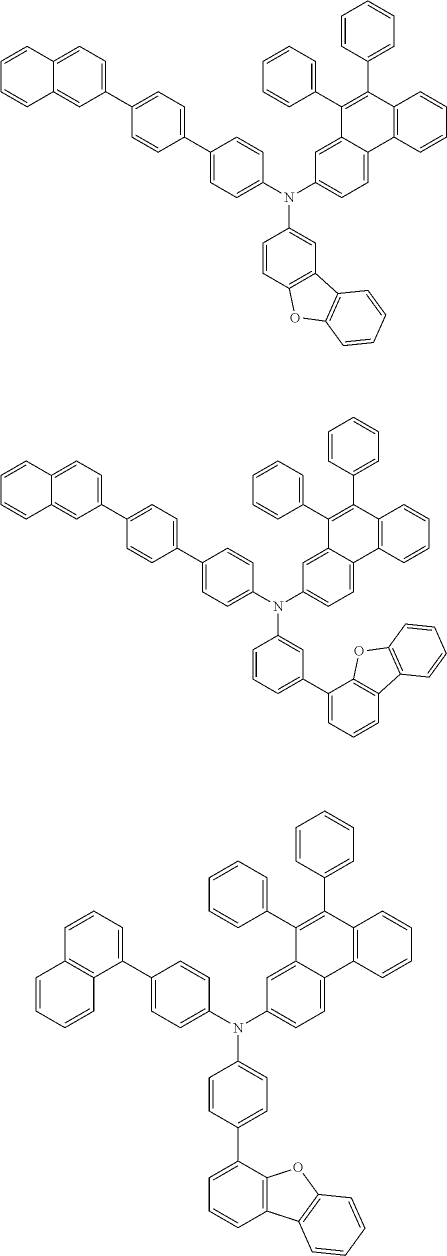 [SMILES: c1ccc(-c2c(-c3ccccc3)c3cc(N(c4ccc(-c5ccc(-c6ccc7ccccc7c6)cc5)cc4)c4ccc5oc6ccccc6c5c4)ccc3c3ccccc23)cc1 ; c1ccc(-c2c(-c3ccccc3)c3cc(N(c4ccc(-c5ccc(-c6ccc7ccccc7c6)cc5)cc4)c4cccc(-c5cccc6c5oc5ccccc56)c4)ccc3c3ccccc23)cc1 ; c1ccc(-c2c(-c3ccccc3)c3cc(N(c4ccc(-c5cccc6ccccc56)cc4)c4ccc(-c5cccc6c5oc5ccccc56)cc4)ccc3c3ccccc23)cc1]

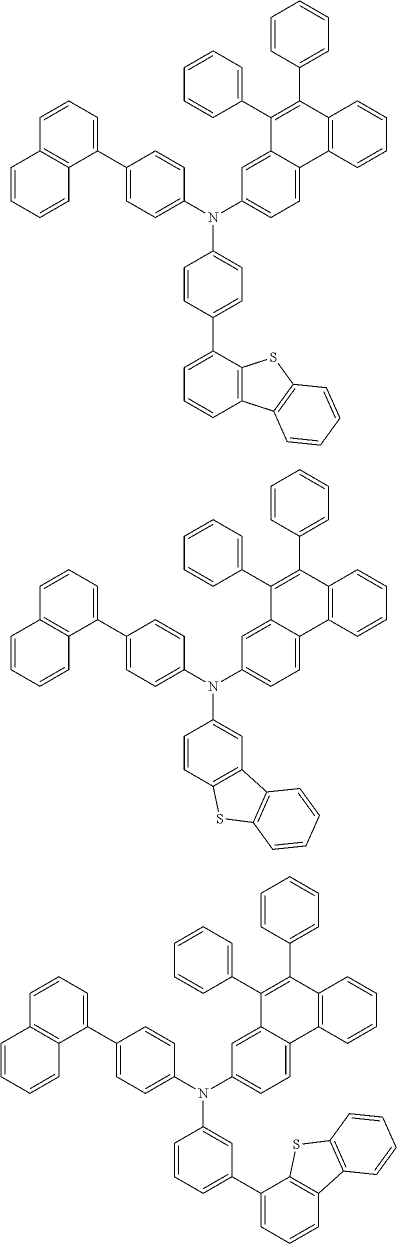

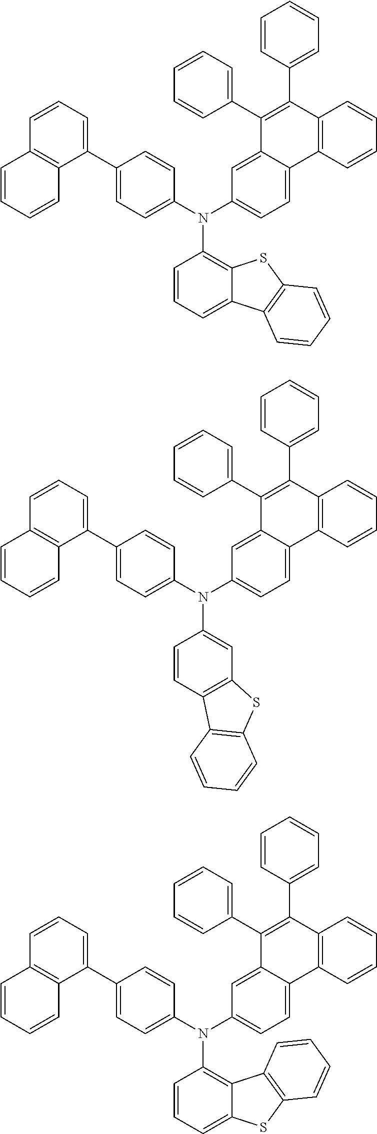

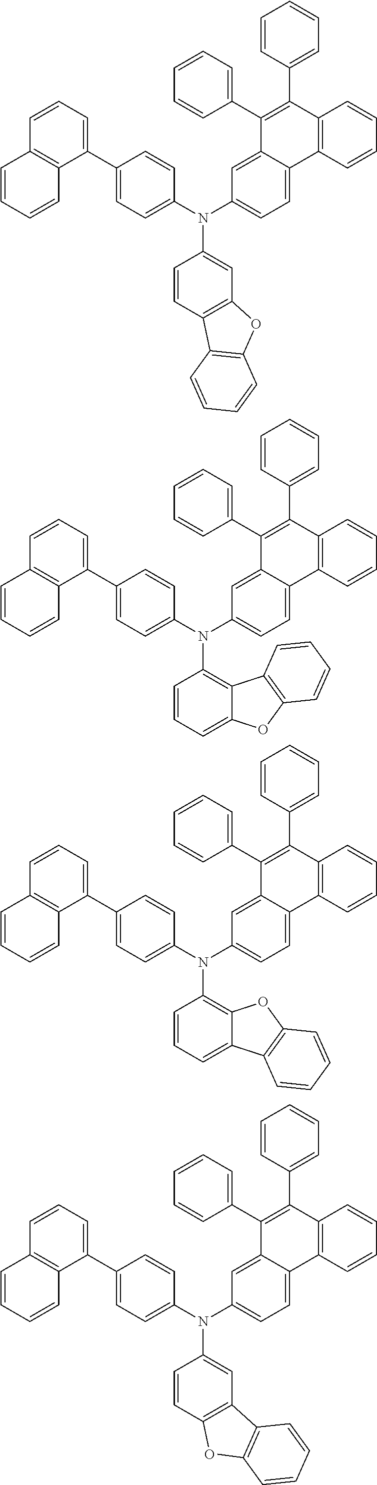

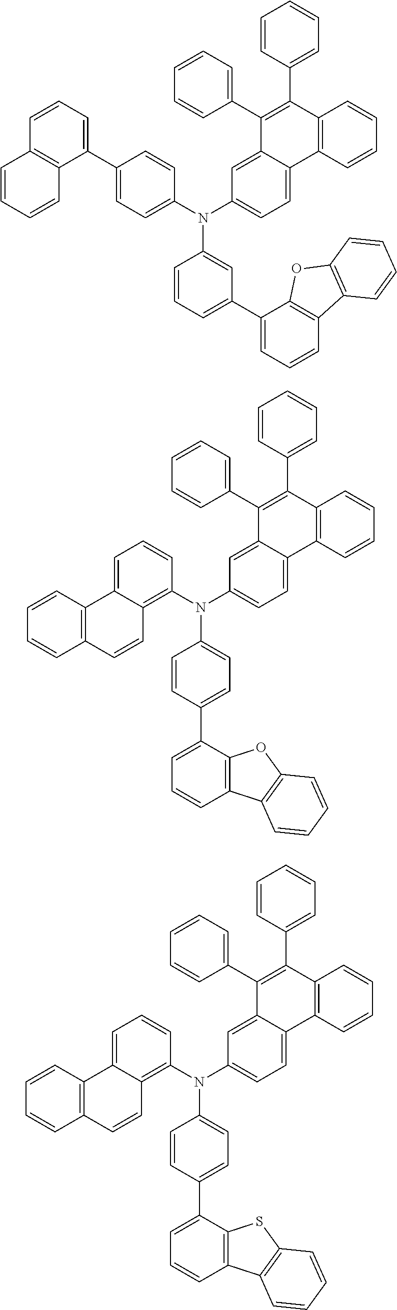

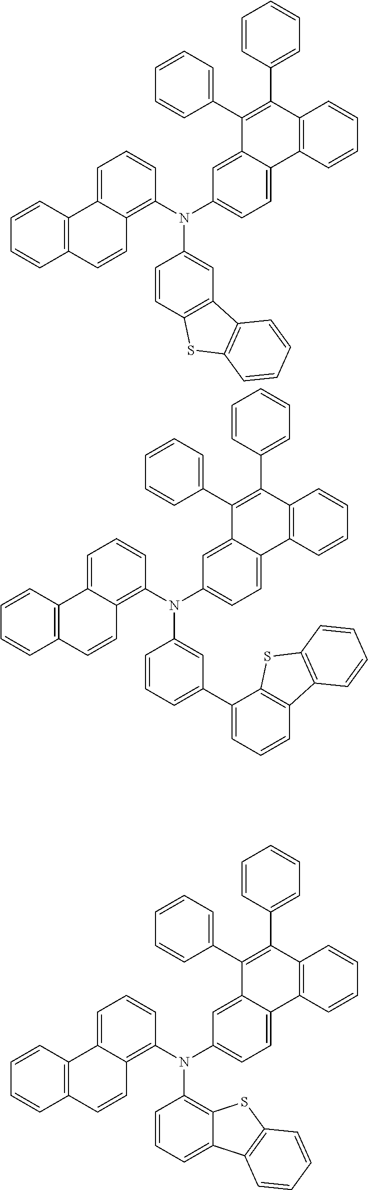

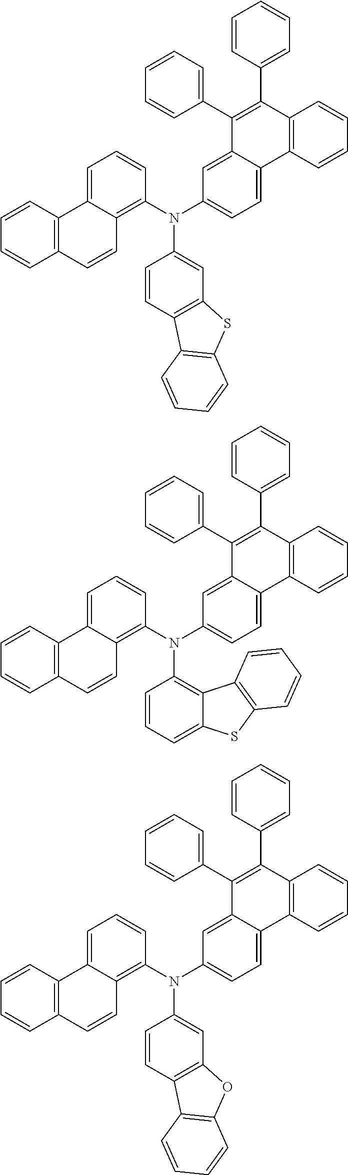

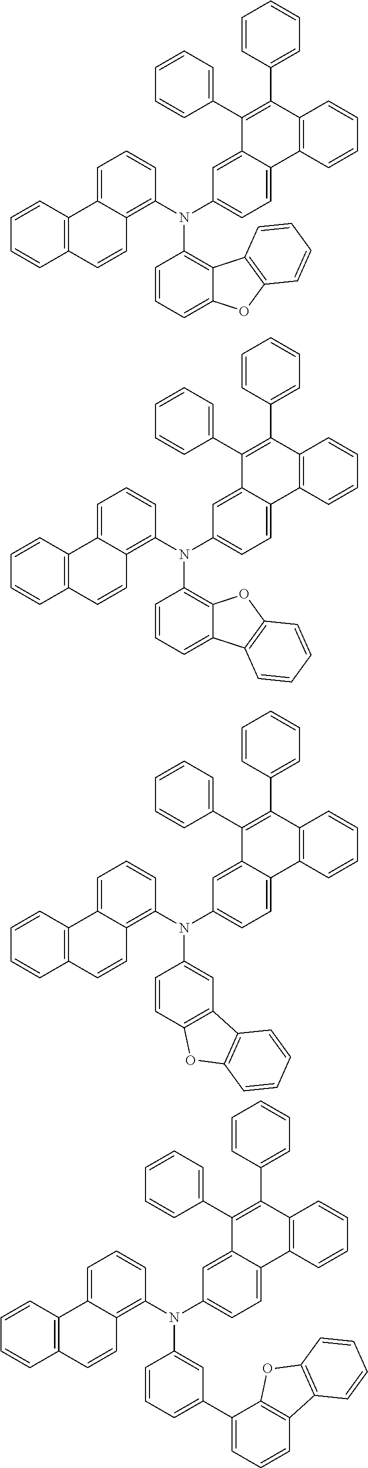

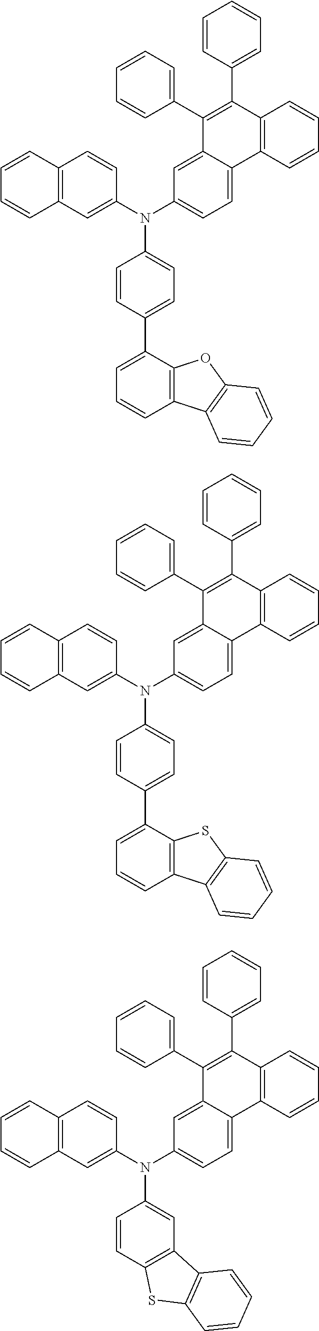

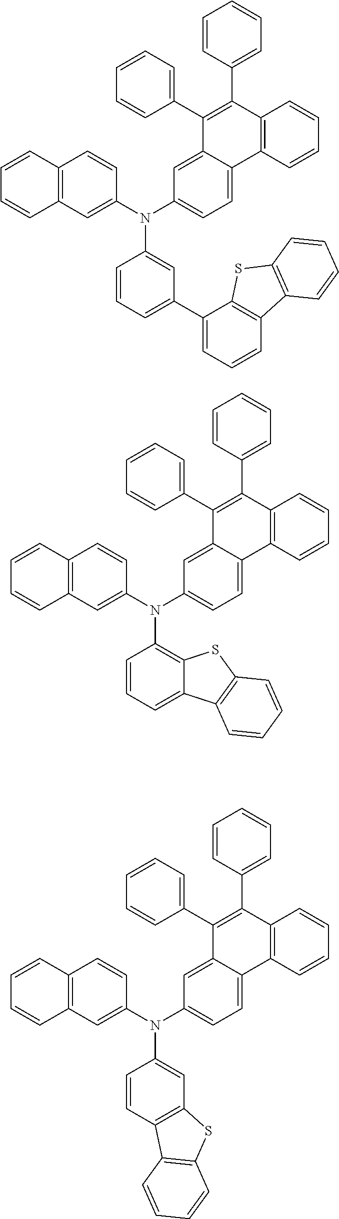

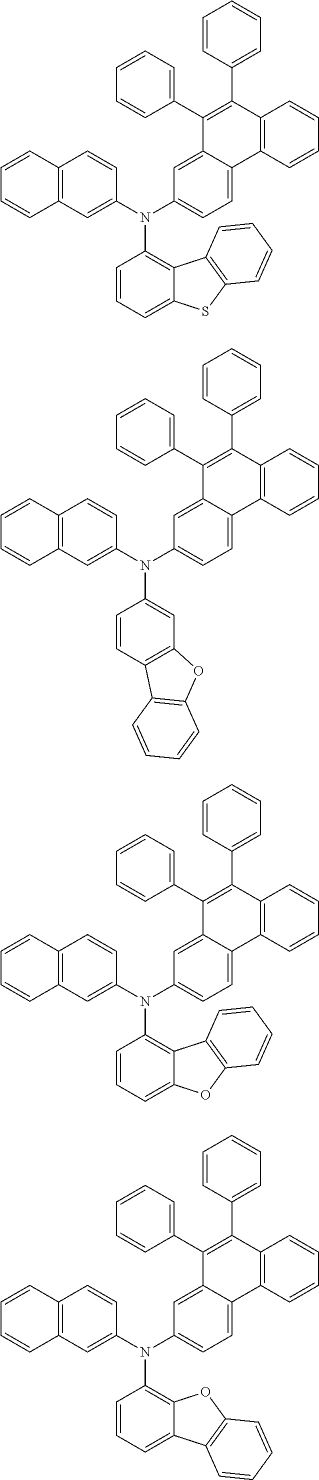

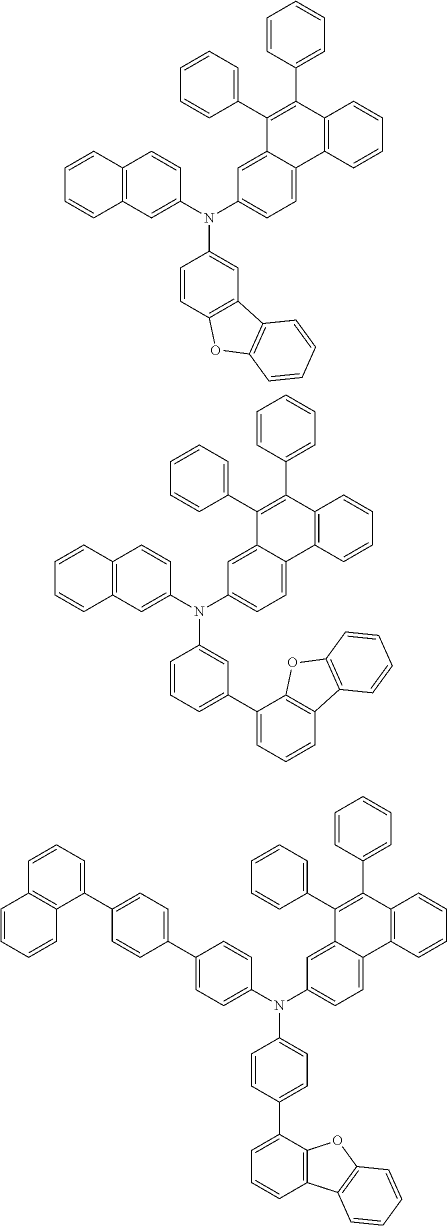

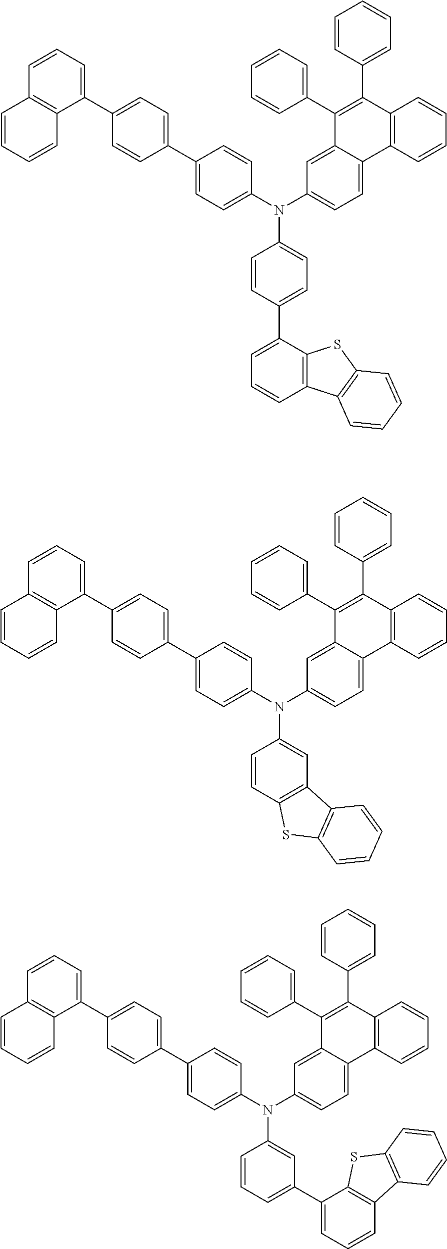 [SMILES: c1ccc(-c2c(-c3ccccc3)c3cc(N(c4ccc(-c5ccc(-c6cccc7ccccc67)cc5)cc4)c4ccc(-c5cccc6c5sc5ccccc56)cc4)ccc3c3ccccc23)cc1 ; c1ccc(-c2c(-c3ccccc3)c3cc(N(c4ccc(-c5ccc(-c6cccc7ccccc67)cc5)cc4)c4ccc5sc6ccccc6c5c4)ccc3c3ccccc23)cc1 ; c1ccc(-c2c(-c3ccccc3)c3cc(N(c4ccc(-c5ccc(-c6cccc7ccccc67)cc5)cc4)c4cccc(-c5cccc6c5sc5ccccc56)c4)ccc3c3ccccc23)cc1]

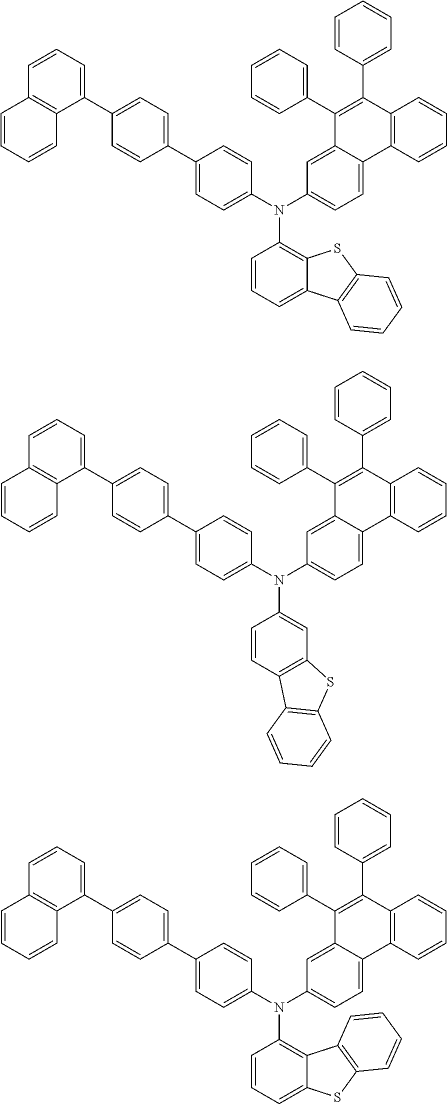

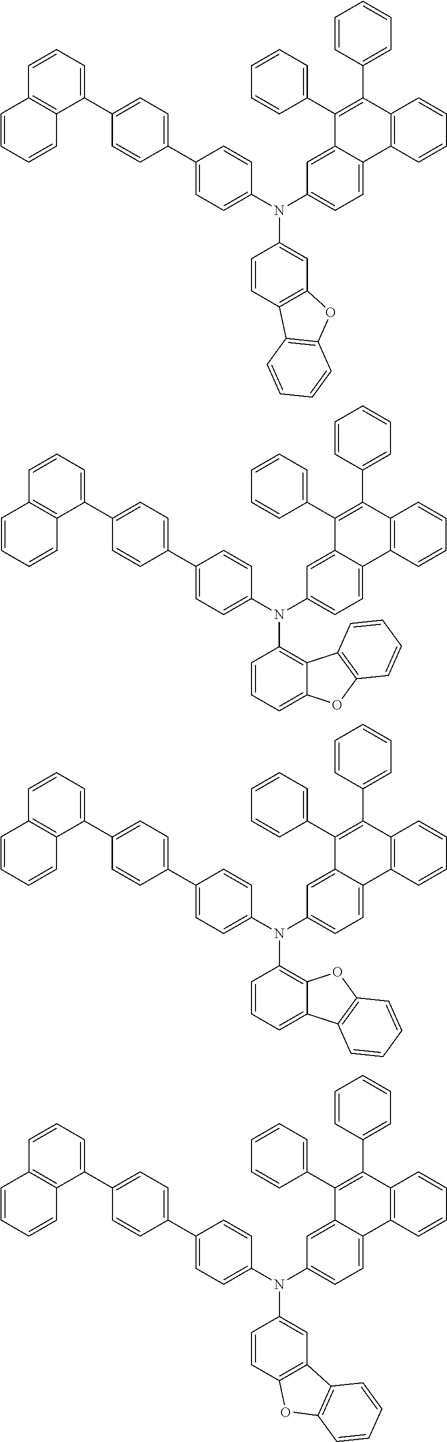

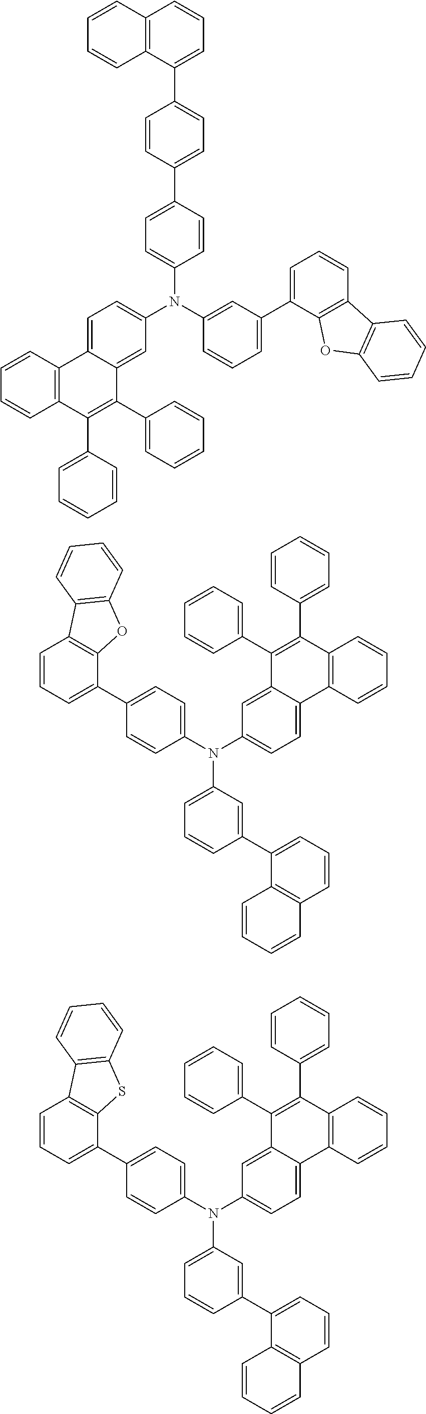

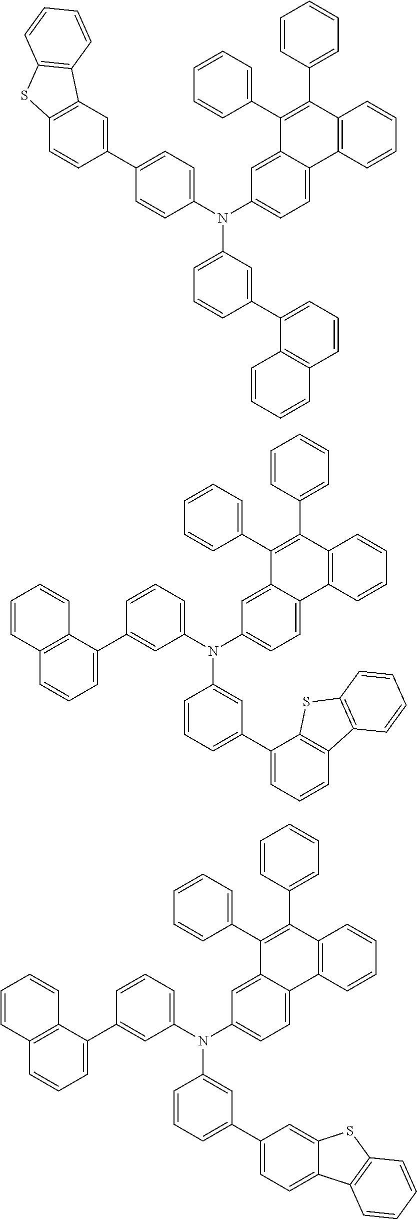

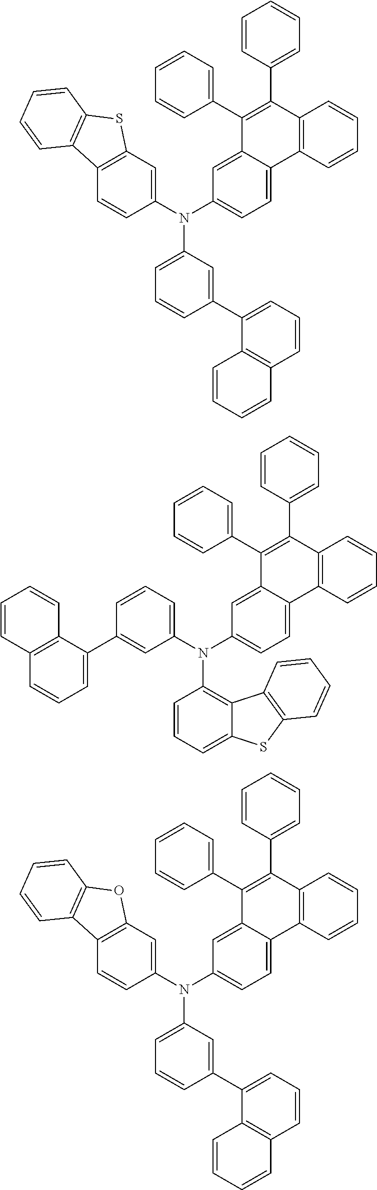

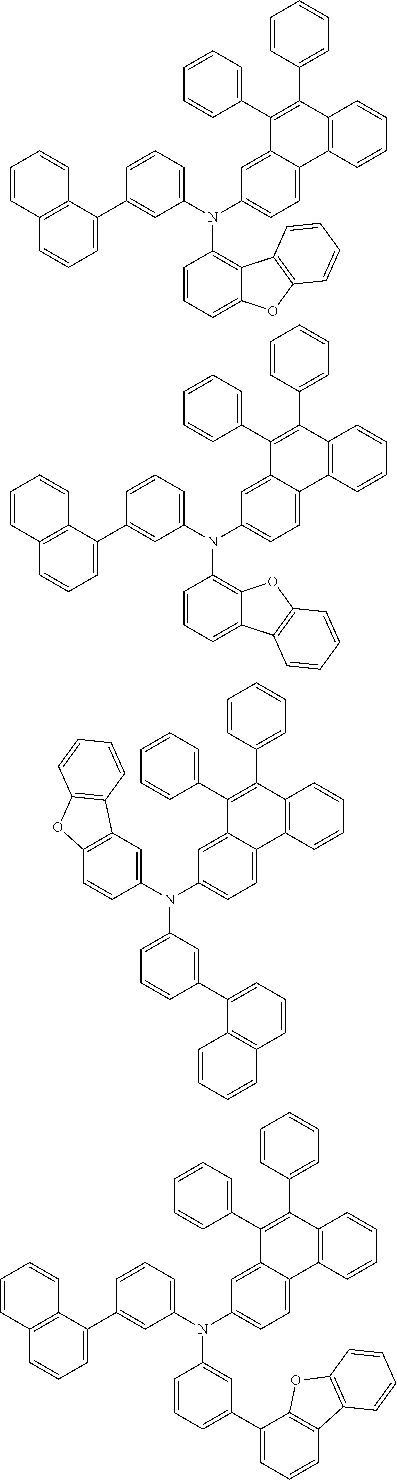

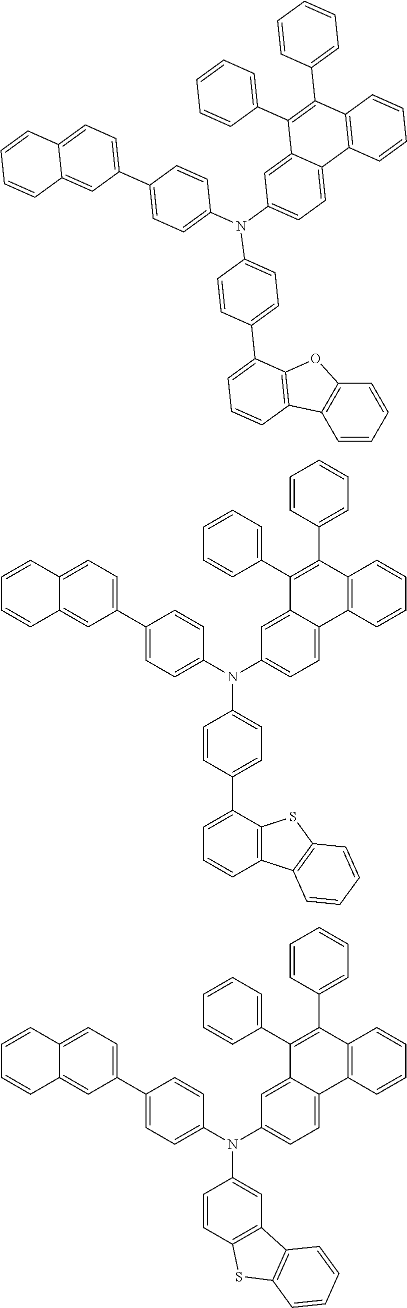

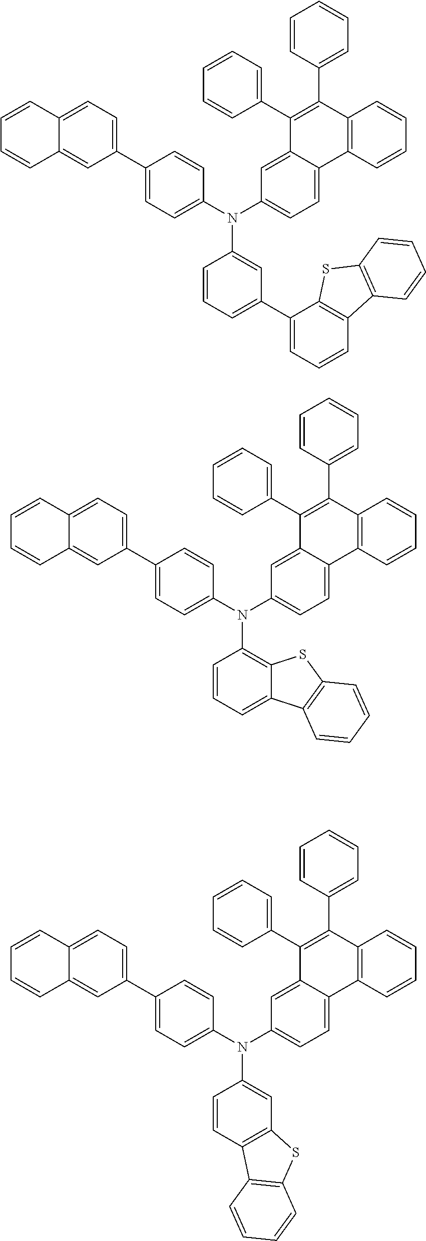

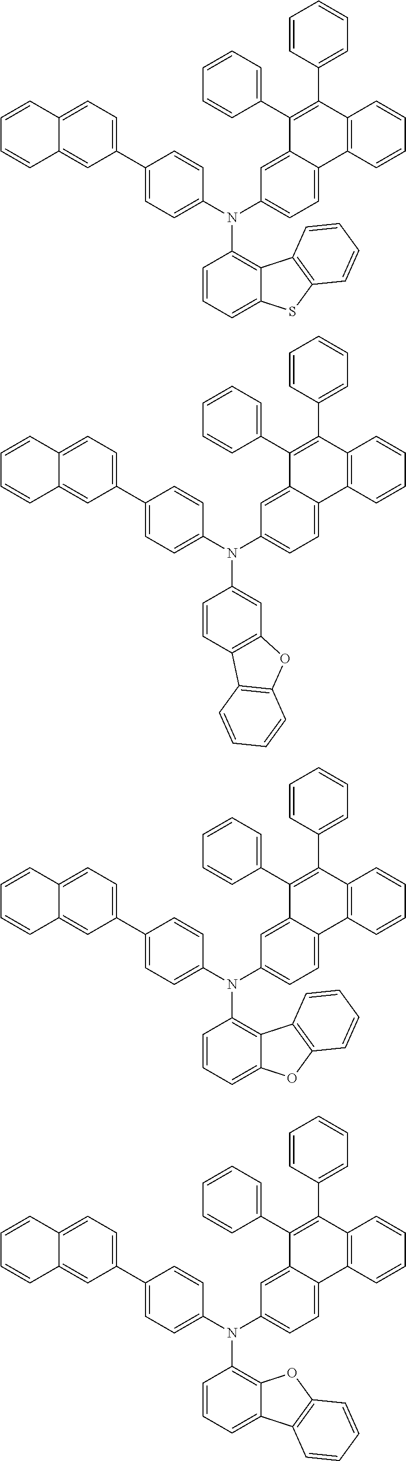

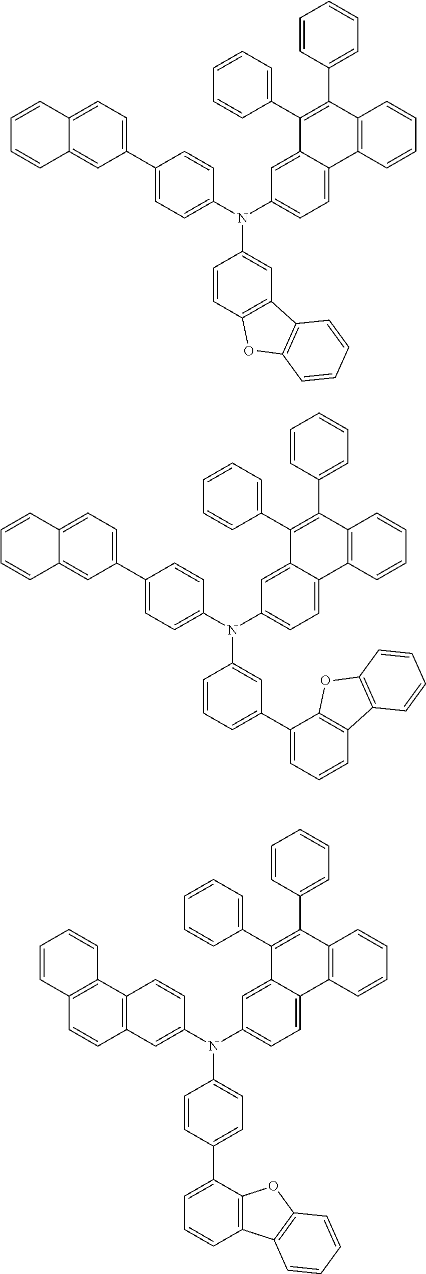

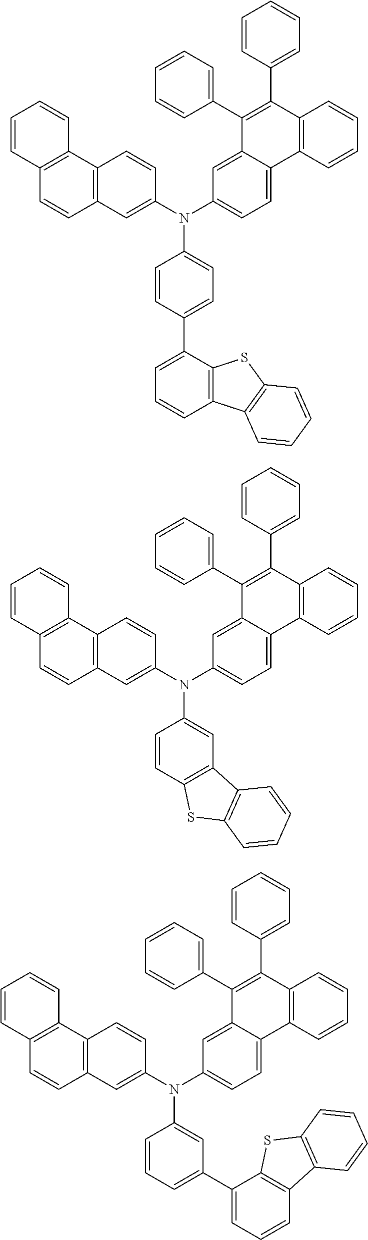

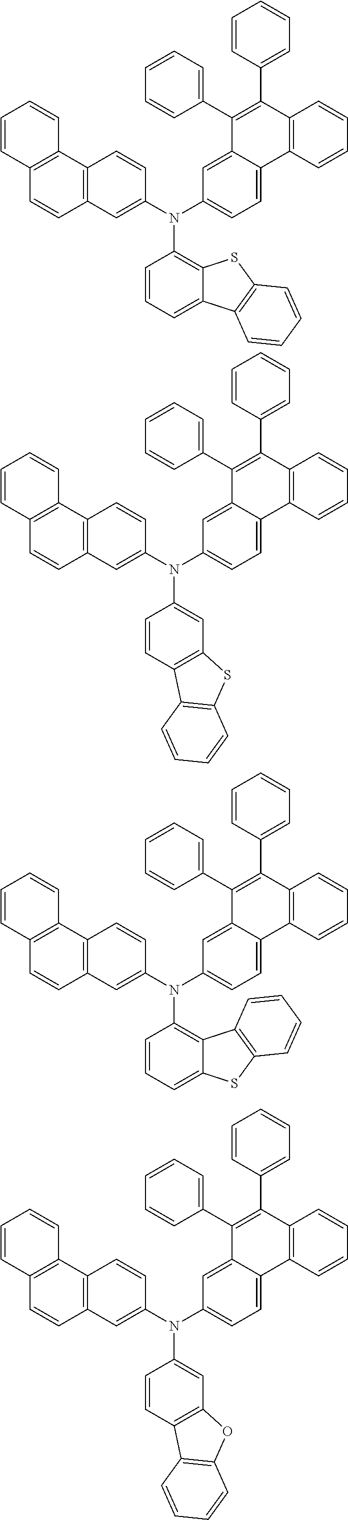

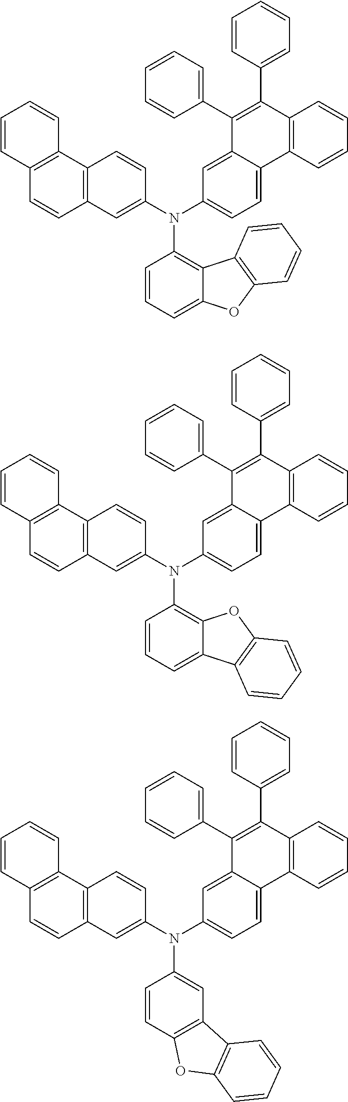

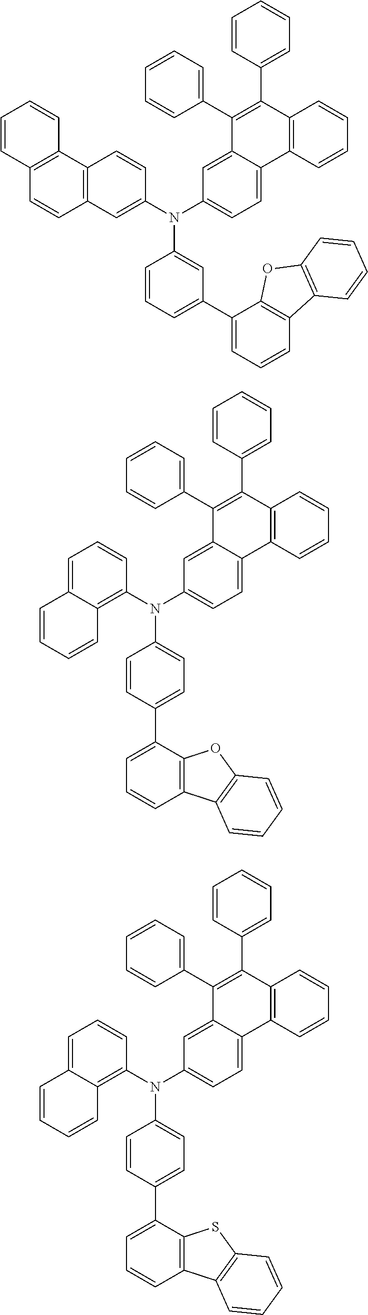

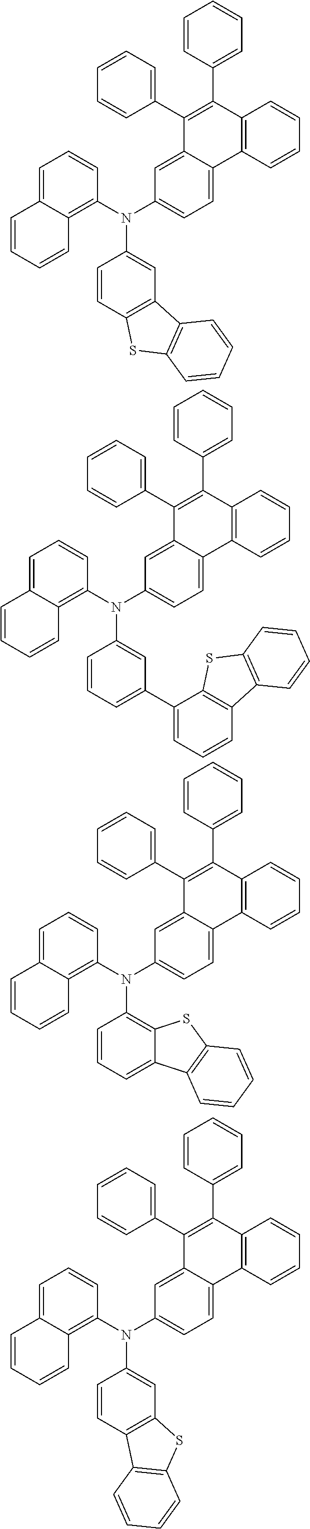

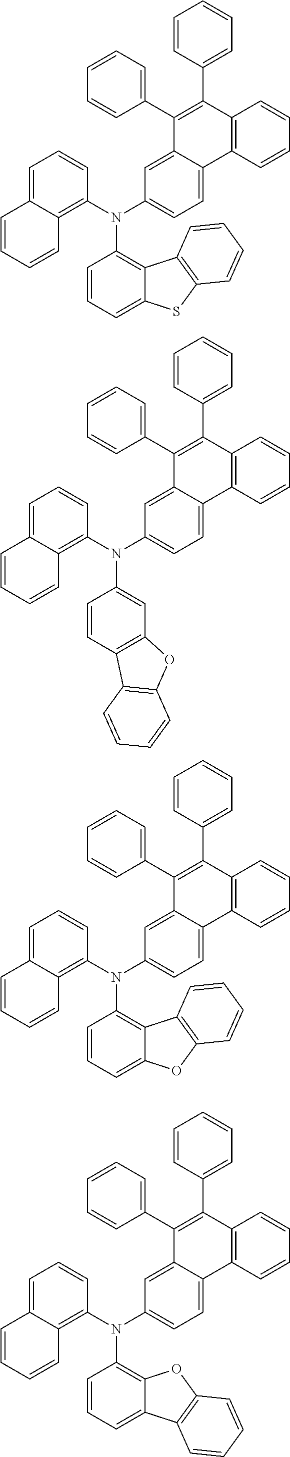

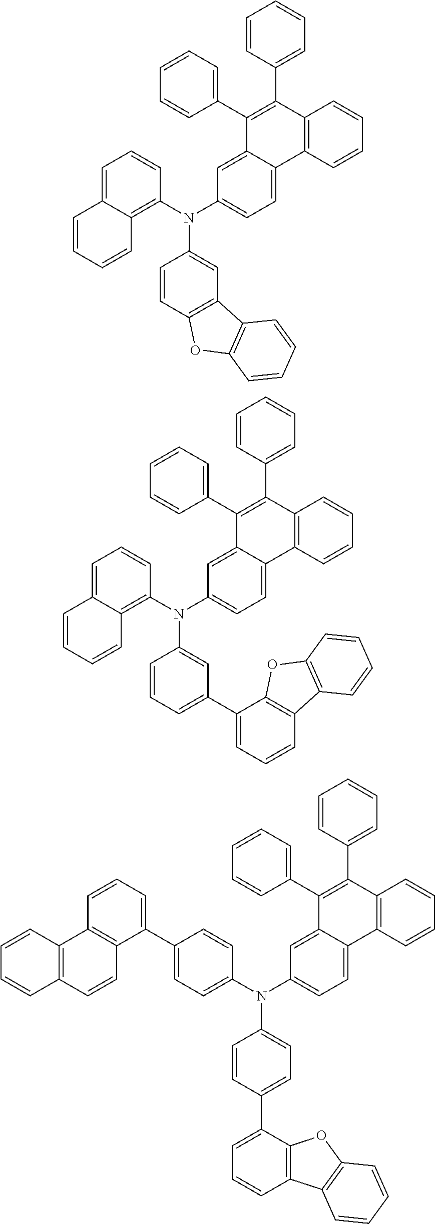

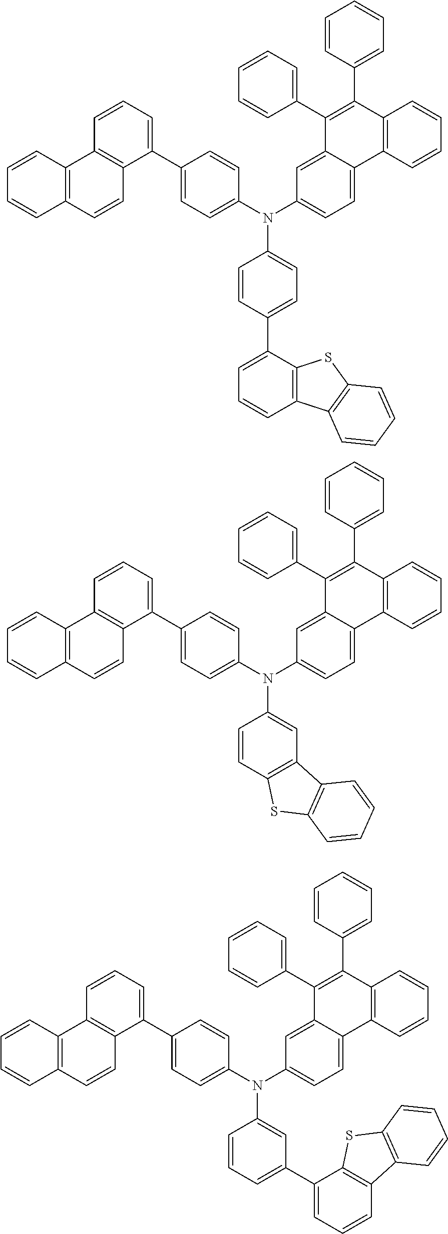

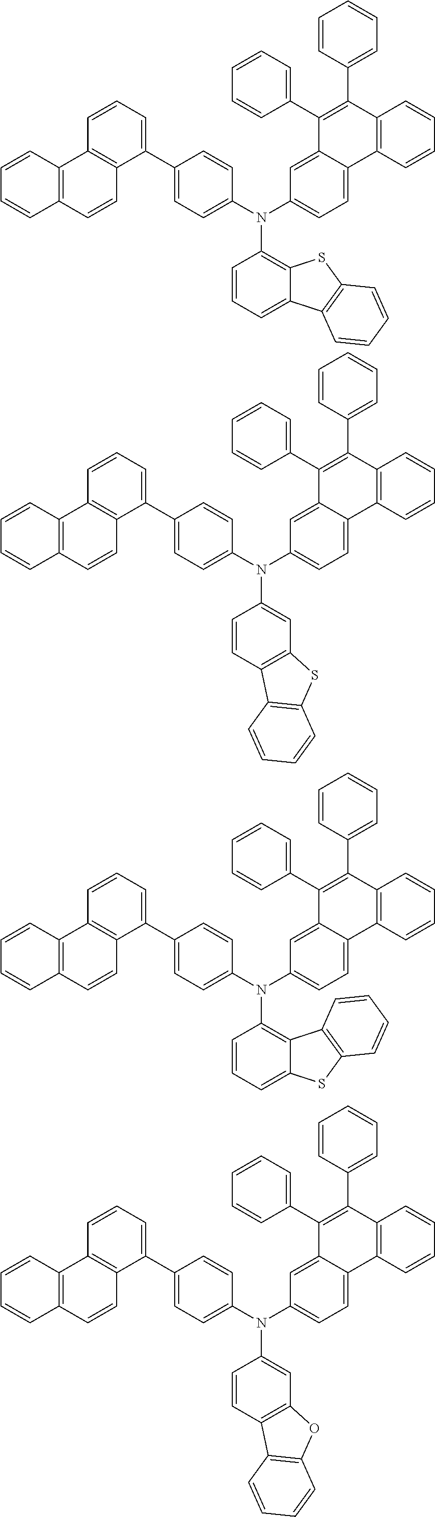

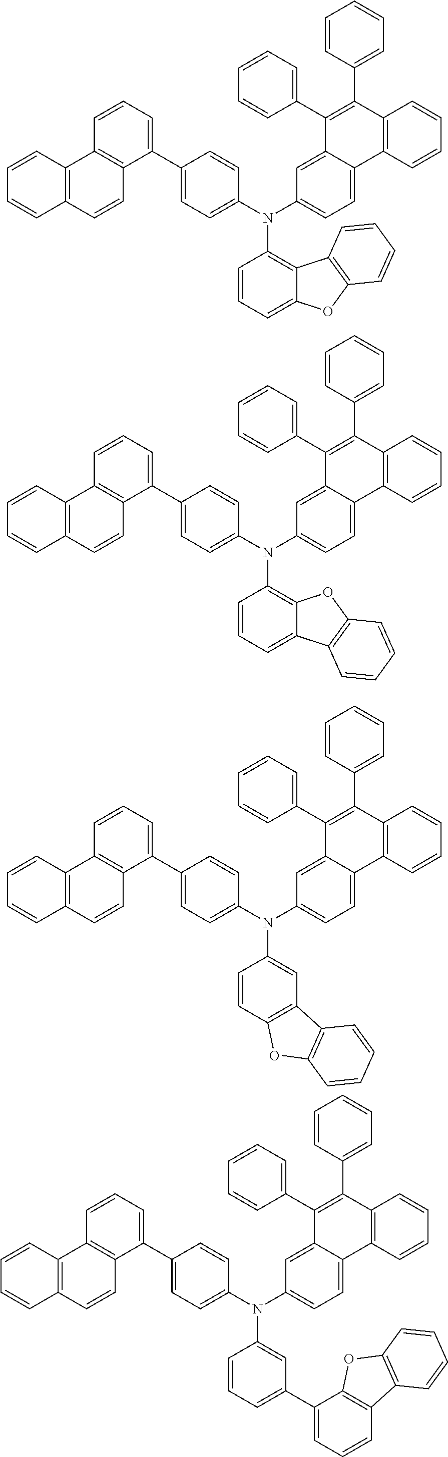

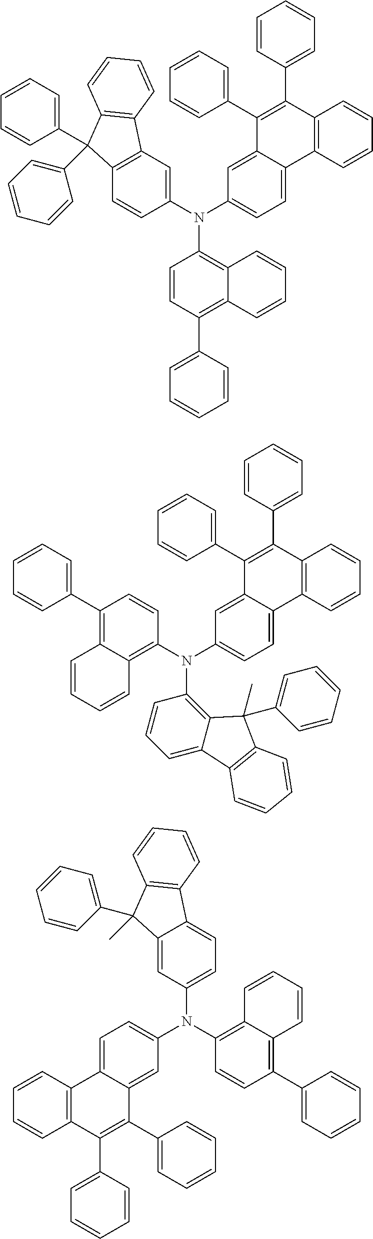 [SMILES: c1ccc(-c2ccc(N(c3ccc4c(c3)-c3ccccc3C4(c3ccccc3)c3ccccc3)c3ccc4c(c3)c(-c3ccccc3)c(-c3ccccc3)c3ccccc34)c3ccccc23)cc1 ; CC1(c2ccccc2)c2ccccc2-c2cccc(N(c3ccc4c(c3)c(-c3ccccc3)c(-c3ccccc3)c3ccccc34)c3ccc(-c4ccccc4)c4ccccc34)c21 ; CC1(c2ccccc2)c2ccccc2-c2ccc(N(c3ccc4c(c3)c(-c3ccccc3)c(-c3ccccc3)c3ccccc34)c3ccc(-c4ccccc4)c4ccccc34)cc21]

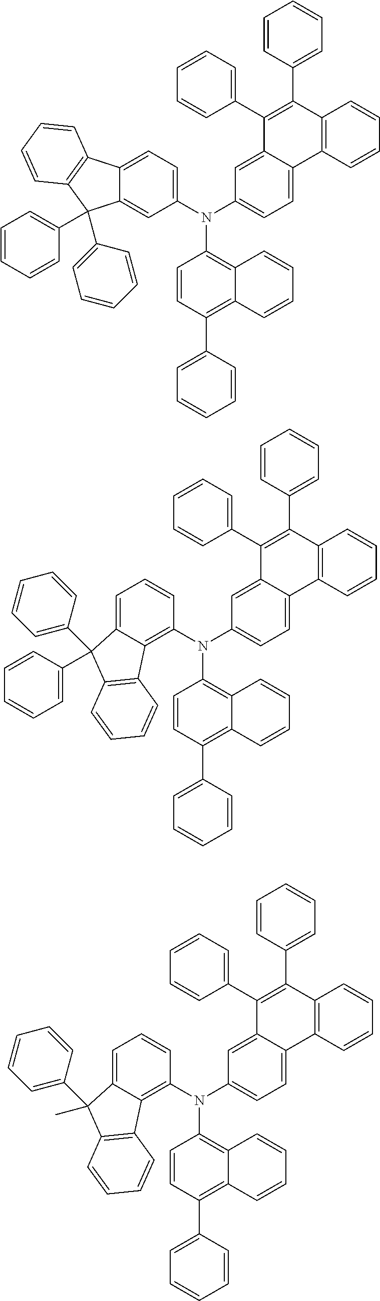 [SMILES: c1ccc(-c2ccc(N(c3ccc4c(c3)C(c3ccccc3)(c3ccccc3)c3ccccc3-4)c3ccc4c(c3)c(-c3ccccc3)c(-c3ccccc3)c3ccccc34)c3ccccc23)cc1 ; c1ccc(-c2ccc(N(c3ccc4c(c3)c(-c3ccccc3)c(-c3ccccc3)c3ccccc34)c3cccc4c3-c3ccccc3C4(c3ccccc3)c3ccccc3)c3ccccc23)cc1 ; CC1(c2ccccc2)c2ccccc2-c2c(N(c3ccc4c(c3)c(-c3ccccc3)c(-c3ccccc3)c3ccccc34)c3ccc(-c4ccccc4)c4ccccc34)cccc21]

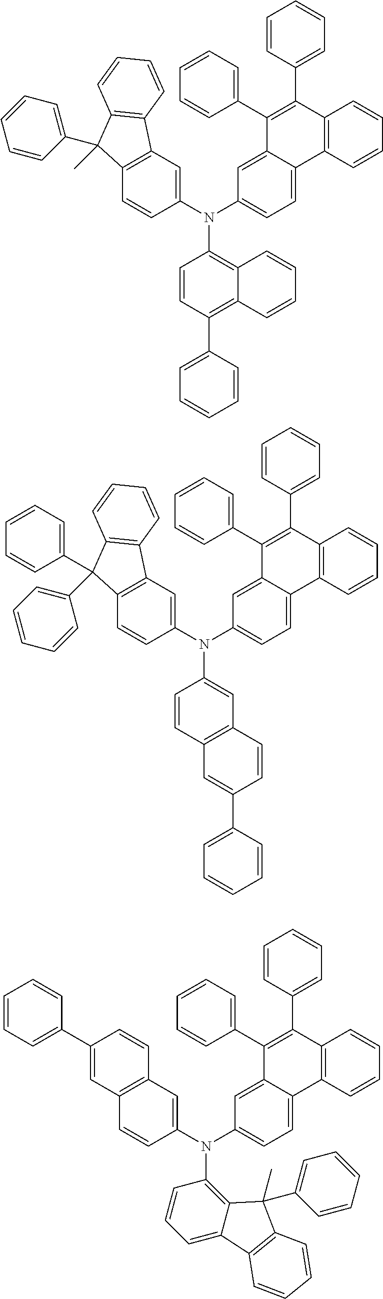 [SMILES: CC1(c2ccccc2)c2ccccc2-c2cc(N(c3ccc4c(c3)c(-c3ccccc3)c(-c3ccccc3)c3ccccc34)c3ccc(-c4ccccc4)c4ccccc34)ccc21 ; c1ccc(-c2ccc3cc(N(c4ccc5c(c4)-c4ccccc4C5(c4ccccc4)c4ccccc4)c4ccc5c(c4)c(-c4ccccc4)c(-c4ccccc4)c4ccccc45)ccc3c2)cc1 ; CC1(c2ccccc2)c2ccccc2-c2cccc(N(c3ccc4cc(-c5ccccc5)ccc4c3)c3ccc4c(c3)c(-c3ccccc3)c(-c3ccccc3)c3ccccc34)c21]

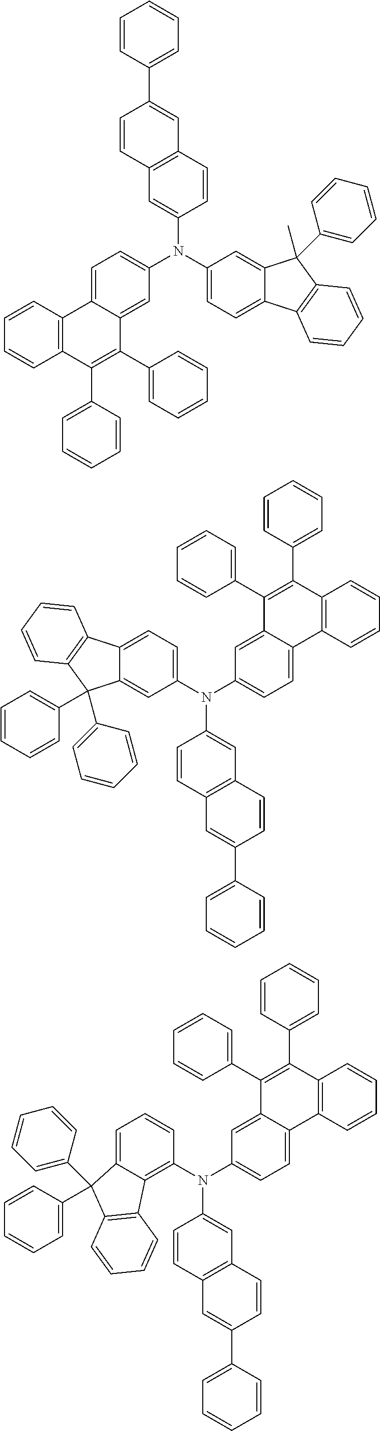

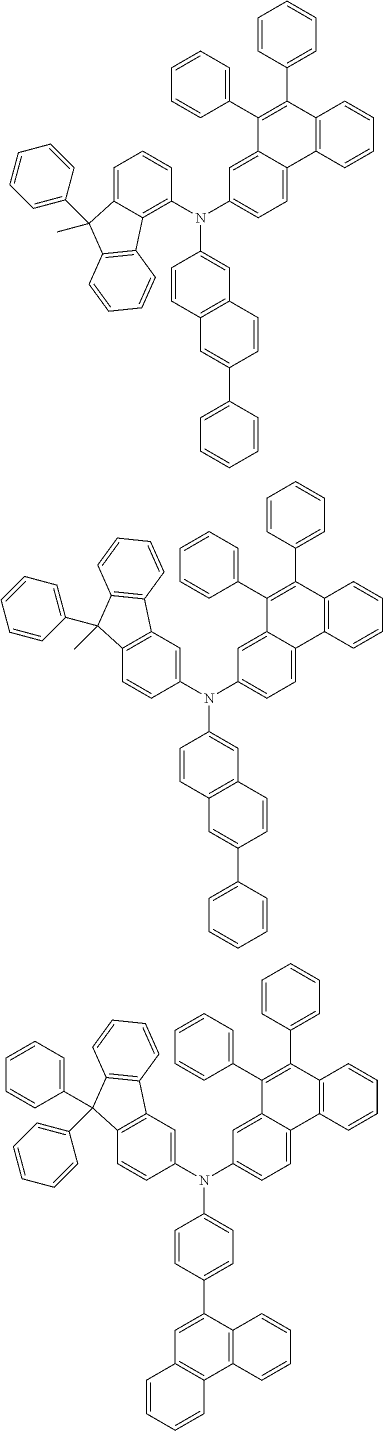

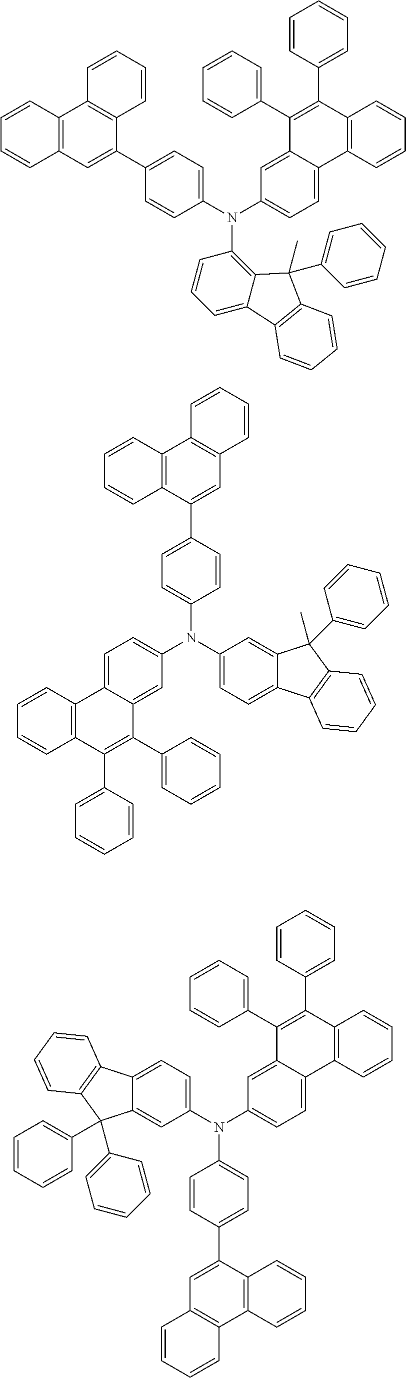 [SMILES: CC1(c2ccccc2)c2ccccc2-c2cccc(N(c3ccc(-c4cc5ccccc5c5ccccc45)cc3)c3ccc4c(c3)c(-c3ccccc3)c(-c3ccccc3)c3ccccc34)c21 ; CC1(c2ccccc2)c2ccccc2-c2ccc(N(c3ccc(-c4cc5ccccc5c5ccccc45)cc3)c3ccc4c(c3)c(-c3ccccc3)c(-c3ccccc3)c3ccccc34)cc21 ; c1ccc(-c2c(-c3ccccc3)c3cc(N(c4ccc(-c5cc6ccccc6c6ccccc56)cc4)c4ccc5c(c4)C(c4ccccc4)(c4ccccc4)c4ccccc4-5)ccc3c3ccccc23)cc1]

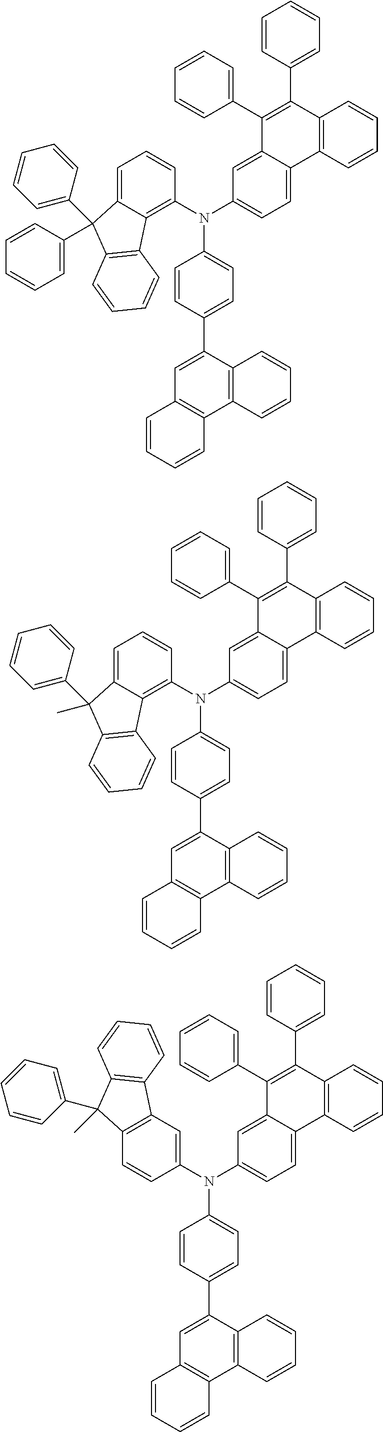 [SMILES: c1ccc(-c2c(-c3ccccc3)c3cc(N(c4ccc(-c5cc6ccccc6c6ccccc56)cc4)c4cccc5c4-c4ccccc4C5(c4ccccc4)c4ccccc4)ccc3c3ccccc23)cc1 ; CC1(c2ccccc2)c2ccccc2-c2c(N(c3ccc(-c4cc5ccccc5c5ccccc45)cc3)c3ccc4c(c3)c(-c3ccccc3)c(-c3ccccc3)c3ccccc34)cccc21 ; CC1(c2ccccc2)c2ccccc2-c2cc(N(c3ccc(-c4cc5ccccc5c5ccccc45)cc3)c3ccc4c(c3)c(-c3ccccc3)c(-c3ccccc3)c3ccccc34)ccc21]

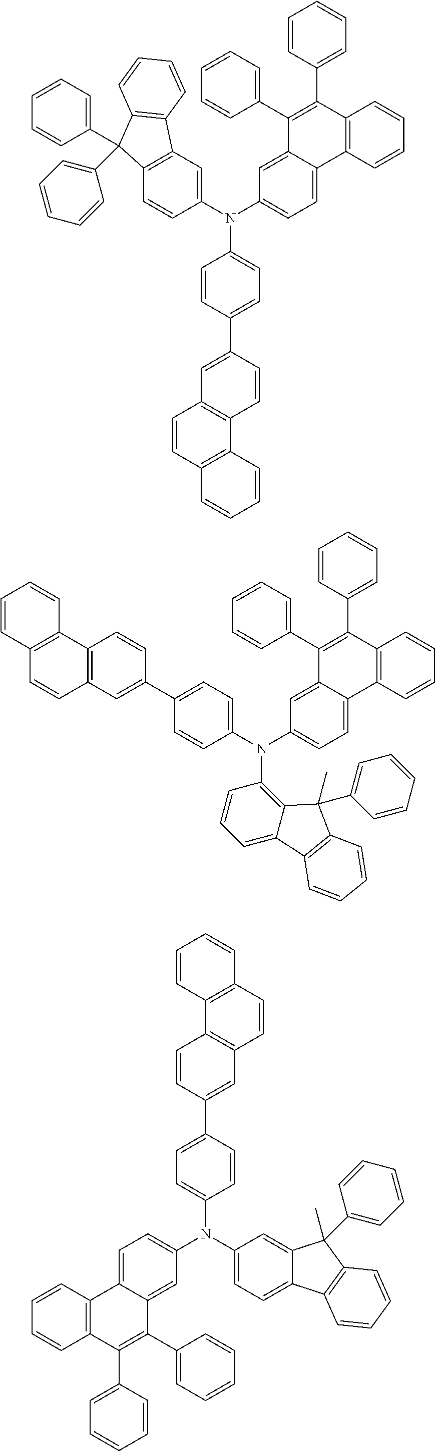 [SMILES: c1ccc(-c2c(-c3ccccc3)c3cc(N(c4ccc(-c5ccc6c(ccc7ccccc76)c5)cc4)c4ccc5c(c4)-c4ccccc4C5(c4ccccc4)c4ccccc4)ccc3c3ccccc23)cc1 ; CC1(c2ccccc2)c2ccccc2-c2cccc(N(c3ccc(-c4ccc5c(ccc6ccccc65)c4)cc3)c3ccc4c(c3)c(-c3ccccc3)c(-c3ccccc3)c3ccccc34)c21 ; CC1(c2ccccc2)c2ccccc2-c2ccc(N(c3ccc(-c4ccc5c(ccc6ccccc65)c4)cc3)c3ccc4c(c3)c(-c3ccccc3)c(-c3ccccc3)c3ccccc34)cc21]

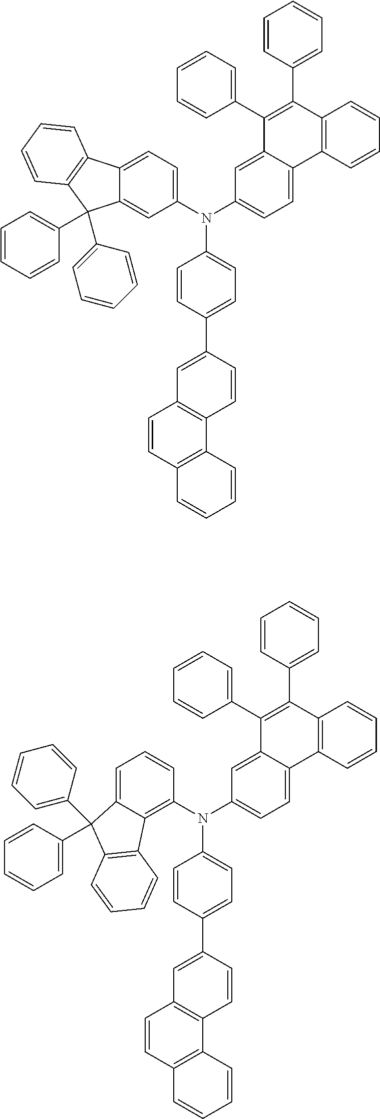 [SMILES: c1ccc(-c2c(-c3ccccc3)c3cc(N(c4ccc(-c5ccc6c(ccc7ccccc76)c5)cc4)c4ccc5c(c4)C(c4ccccc4)(c4ccccc4)c4ccccc4-5)ccc3c3ccccc23)cc1 ; c1ccc(-c2c(-c3ccccc3)c3cc(N(c4ccc(-c5ccc6c(ccc7ccccc76)c5)cc4)c4cccc5c4-c4ccccc4C5(c4ccccc4)c4ccccc4)ccc3c3ccccc23)cc1]

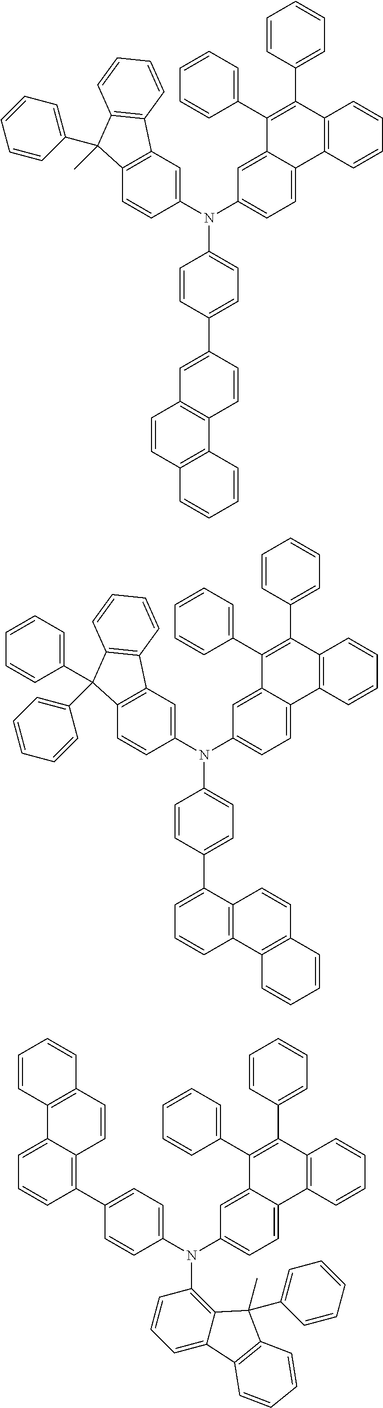

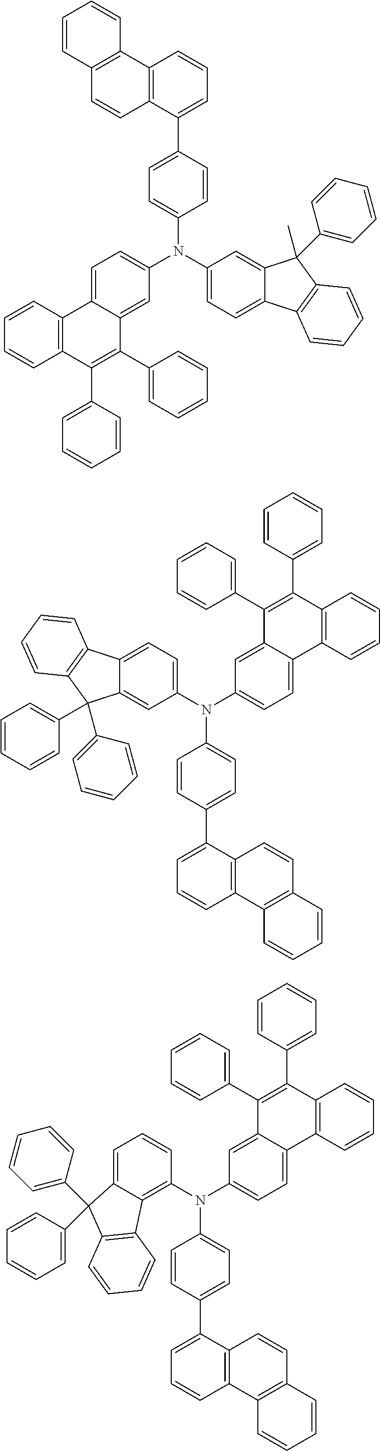

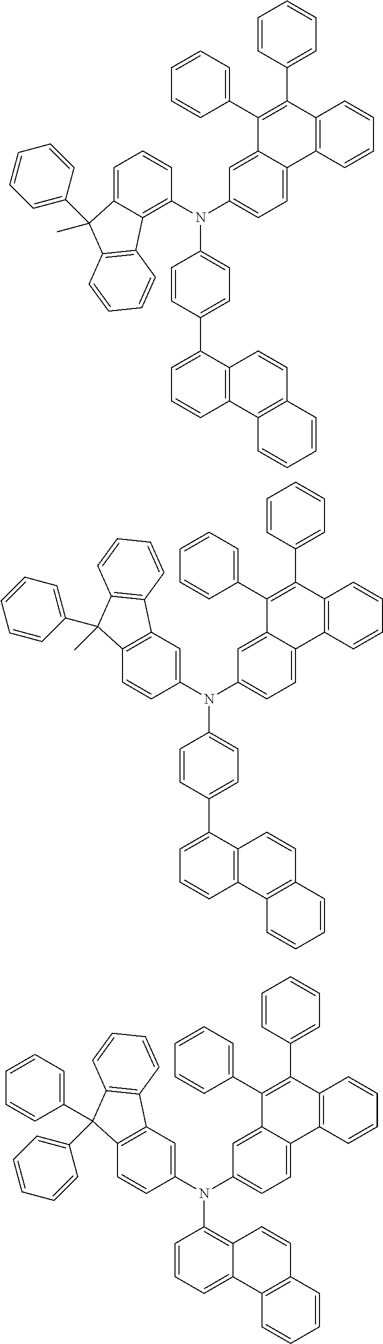

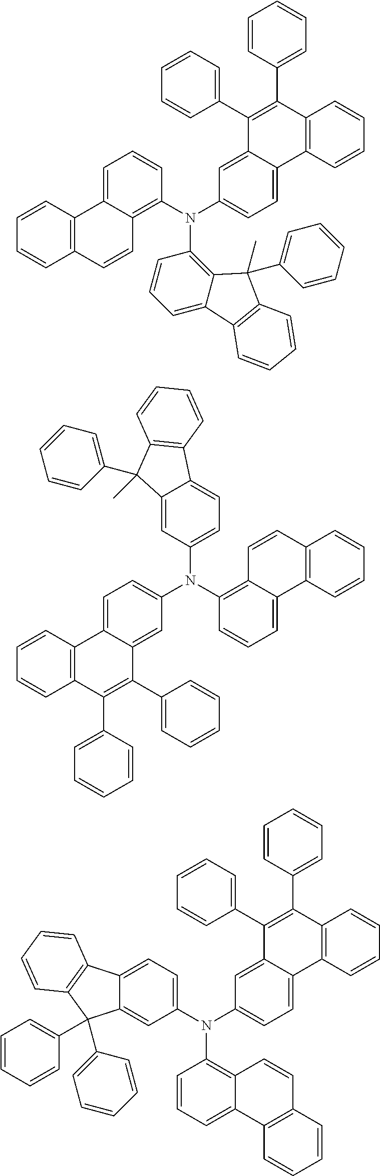 [SMILES: CC1(c2ccccc2)c2ccccc2-c2cccc(N(c3ccc4c(c3)c(-c3ccccc3)c(-c3ccccc3)c3ccccc34)c3cccc4c3ccc3ccccc34)c21 ; CC1(c2ccccc2)c2ccccc2-c2ccc(N(c3ccc4c(c3)c(-c3ccccc3)c(-c3ccccc3)c3ccccc34)c3cccc4c3ccc3ccccc34)cc21 ; c1ccc(-c2c(-c3ccccc3)c3cc(N(c4ccc5c(c4)C(c4ccccc4)(c4ccccc4)c4ccccc4-5)c4cccc5c4ccc4ccccc45)ccc3c3ccccc23)cc1]

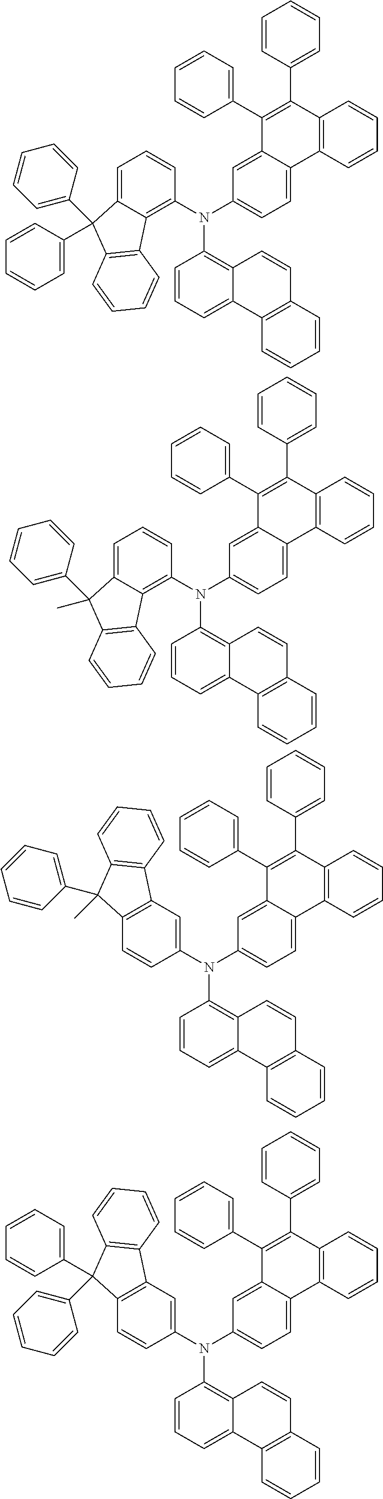

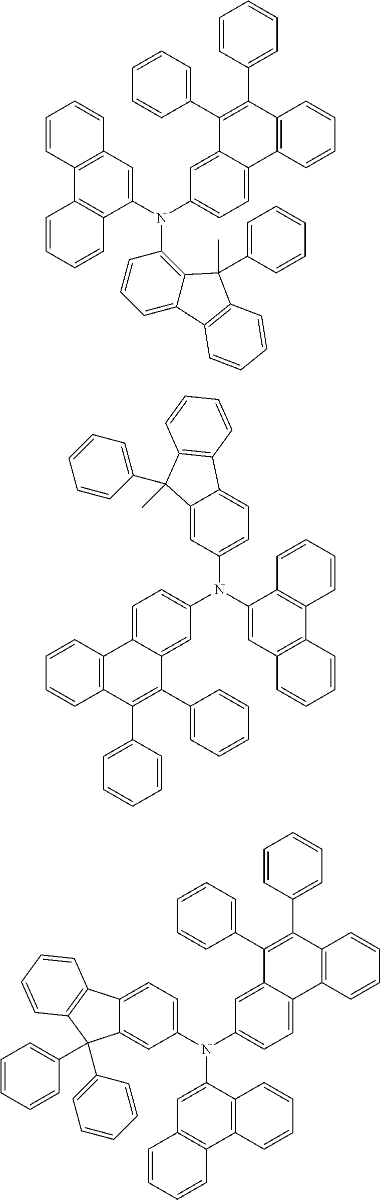

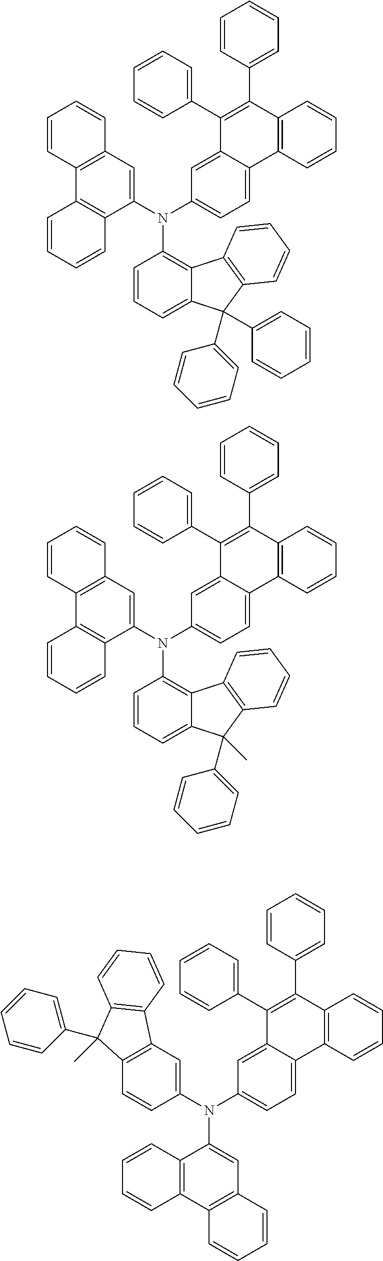

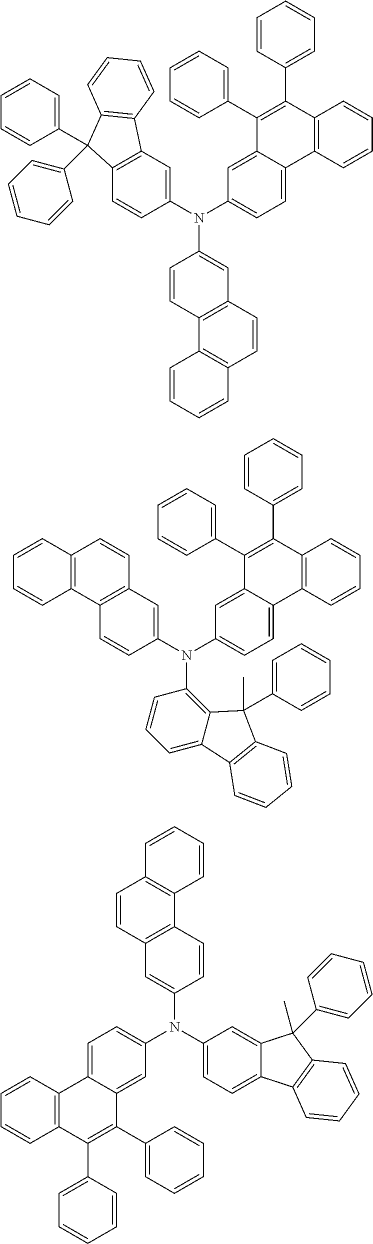 [SMILES: c1ccc(-c2c(-c3ccccc3)c3cc(N(c4ccc5c(c4)-c4ccccc4C5(c4ccccc4)c4ccccc4)c4ccc5c(ccc6ccccc65)c4)ccc3c3ccccc23)cc1 ; CC1(c2ccccc2)c2ccccc2-c2cccc(N(c3ccc4c(ccc5ccccc54)c3)c3ccc4c(c3)c(-c3ccccc3)c(-c3ccccc3)c3ccccc34)c21 ; CC1(c2ccccc2)c2ccccc2-c2ccc(N(c3ccc4c(ccc5ccccc54)c3)c3ccc4c(c3)c(-c3ccccc3)c(-c3ccccc3)c3ccccc34)cc21]

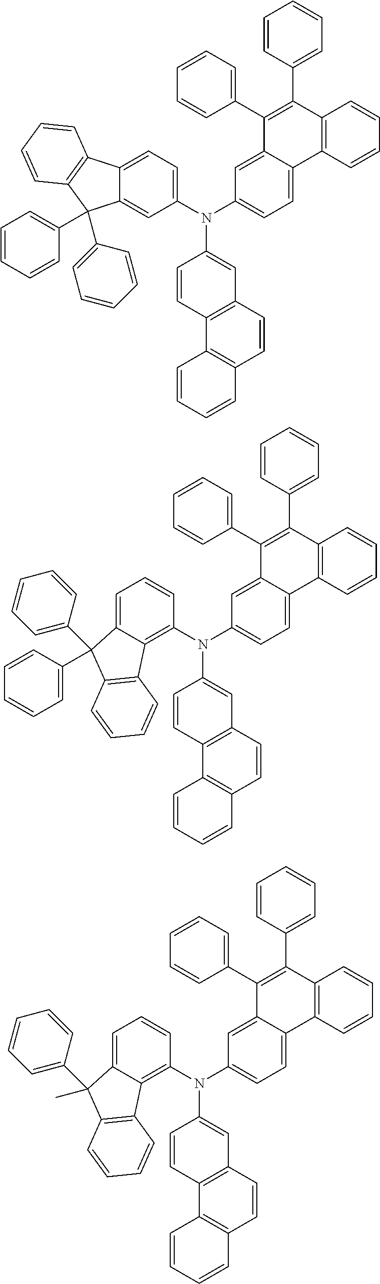 [SMILES: c1ccc(-c2c(-c3ccccc3)c3cc(N(c4ccc5c(c4)C(c4ccccc4)(c4ccccc4)c4ccccc4-5)c4ccc5c(ccc6ccccc65)c4)ccc3c3ccccc23)cc1 ; c1ccc(-c2c(-c3ccccc3)c3cc(N(c4ccc5c(ccc6ccccc65)c4)c4cccc5c4-c4ccccc4C5(c4ccccc4)c4ccccc4)ccc3c3ccccc23)cc1 ; CC1(c2ccccc2)c2ccccc2-c2c(N(c3ccc4c(ccc5ccccc54)c3)c3ccc4c(c3)c(-c3ccccc3)c(-c3ccccc3)c3ccccc34)cccc21]

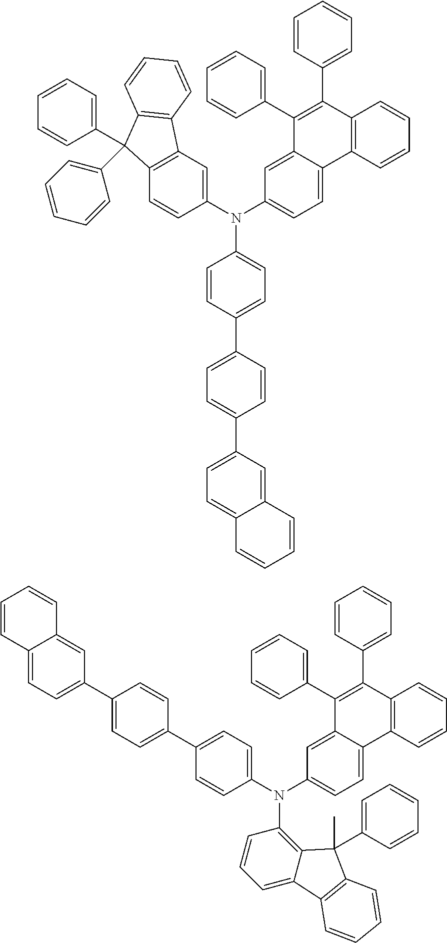 [SMILES: c1ccc(-c2c(-c3ccccc3)c3cc(N(c4ccc(-c5ccc(-c6ccc7ccccc7c6)cc5)cc4)c4ccc5c(c4)-c4ccccc4C5(c4ccccc4)c4ccccc4)ccc3c3ccccc23)cc1 ; CC1(c2ccccc2)c2ccccc2-c2cccc(N(c3ccc(-c4ccc(-c5ccc6ccccc6c5)cc4)cc3)c3ccc4c(c3)c(-c3ccccc3)c(-c3ccccc3)c3ccccc34)c21]

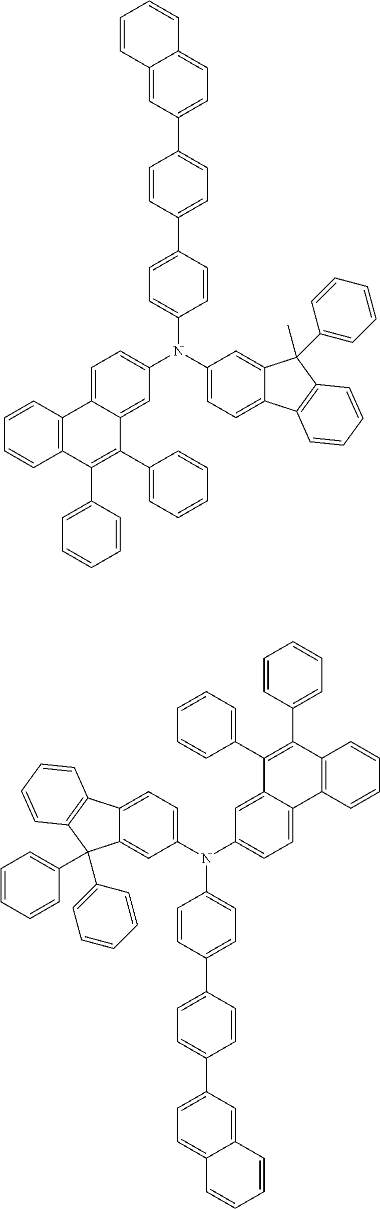 [SMILES: CC1(c2ccccc2)c2ccccc2-c2ccc(N(c3ccc(-c4ccc(-c5ccc6ccccc6c5)cc4)cc3)c3ccc4c(c3)c(-c3ccccc3)c(-c3ccccc3)c3ccccc34)cc21 ; c1ccc(-c2c(-c3ccccc3)c3cc(N(c4ccc(-c5ccc(-c6ccc7ccccc7c6)cc5)cc4)c4ccc5c(c4)C(c4ccccc4)(c4ccccc4)c4ccccc4-5)ccc3c3ccccc23)cc1]

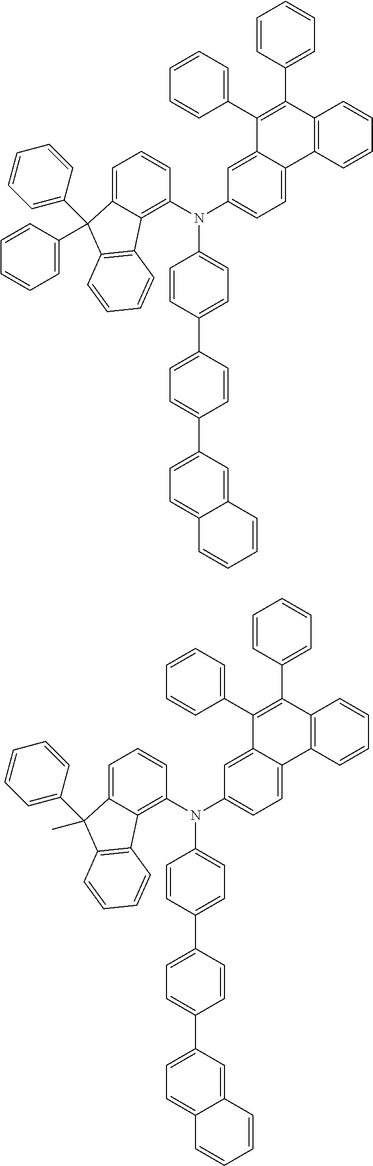 [SMILES: c1ccc(-c2c(-c3ccccc3)c3cc(N(c4ccc(-c5ccc(-c6ccc7ccccc7c6)cc5)cc4)c4cccc5c4-c4ccccc4C5(c4ccccc4)c4ccccc4)ccc3c3ccccc23)cc1 ; CC1(c2ccccc2)c2ccccc2-c2c(N(c3ccc(-c4ccc(-c5ccc6ccccc6c5)cc4)cc3)c3ccc4c(c3)c(-c3ccccc3)c(-c3ccccc3)c3ccccc34)cccc21]

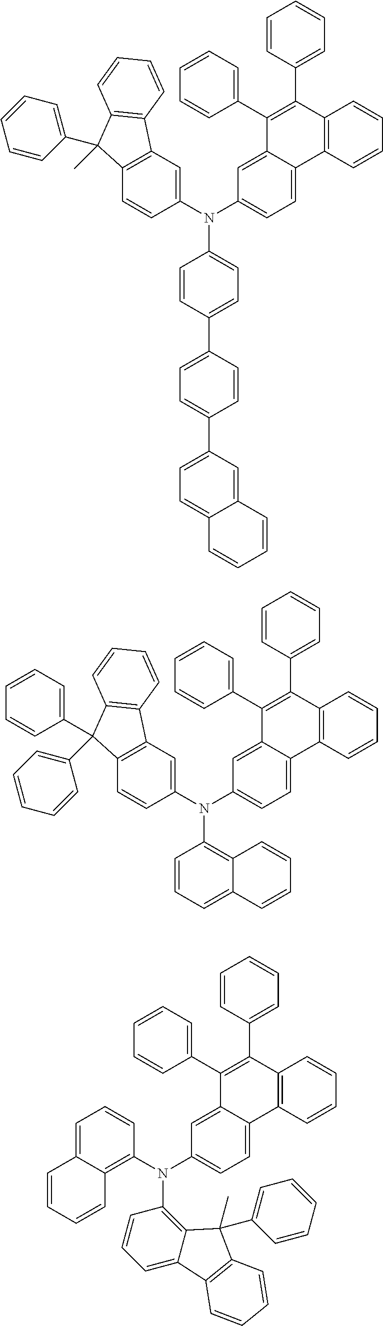 [SMILES: CC1(c2ccccc2)c2ccccc2-c2cc(N(c3ccc(-c4ccc(-c5ccc6ccccc6c5)cc4)cc3)c3ccc4c(c3)c(-c3ccccc3)c(-c3ccccc3)c3ccccc34)ccc21 ; c1ccc(-c2c(-c3ccccc3)c3cc(N(c4ccc5c(c4)-c4ccccc4C5(c4ccccc4)c4ccccc4)c4cccc5ccccc45)ccc3c3ccccc23)cc1 ; CC1(c2ccccc2)c2ccccc2-c2cccc(N(c3ccc4c(c3)c(-c3ccccc3)c(-c3ccccc3)c3ccccc34)c3cccc4ccccc34)c21]

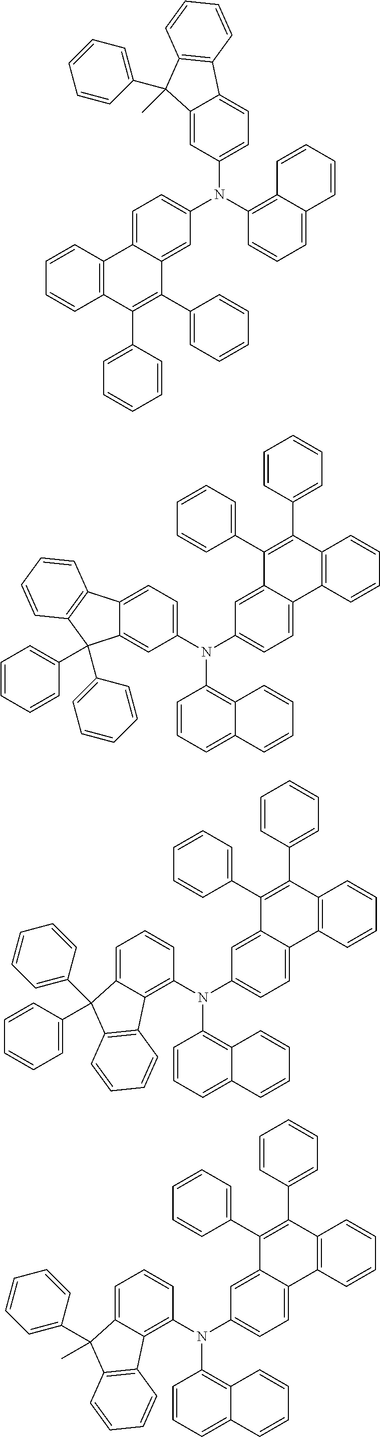

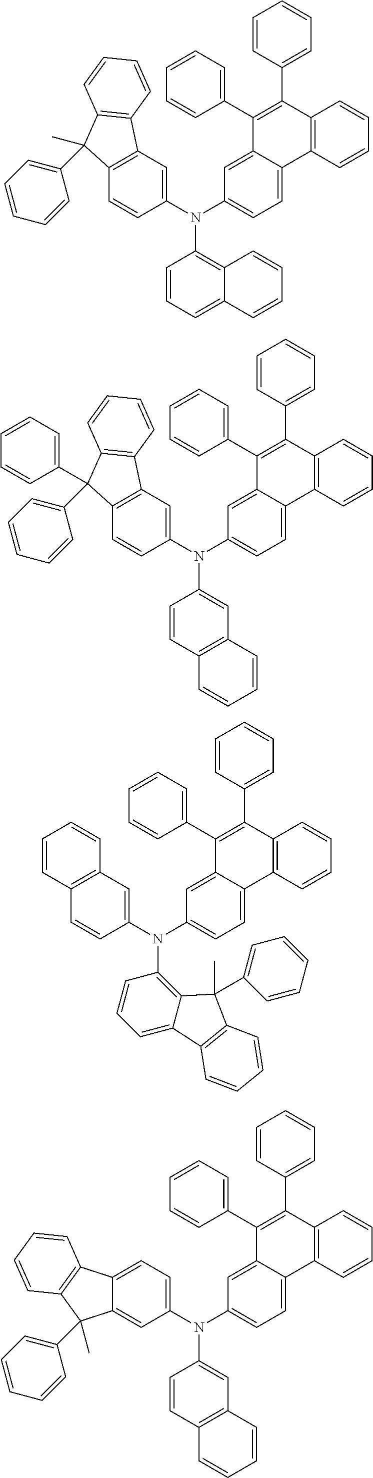

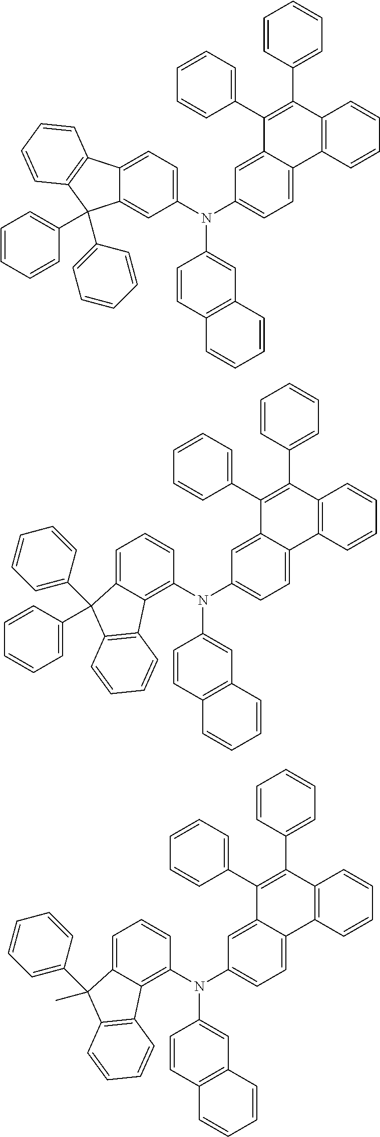

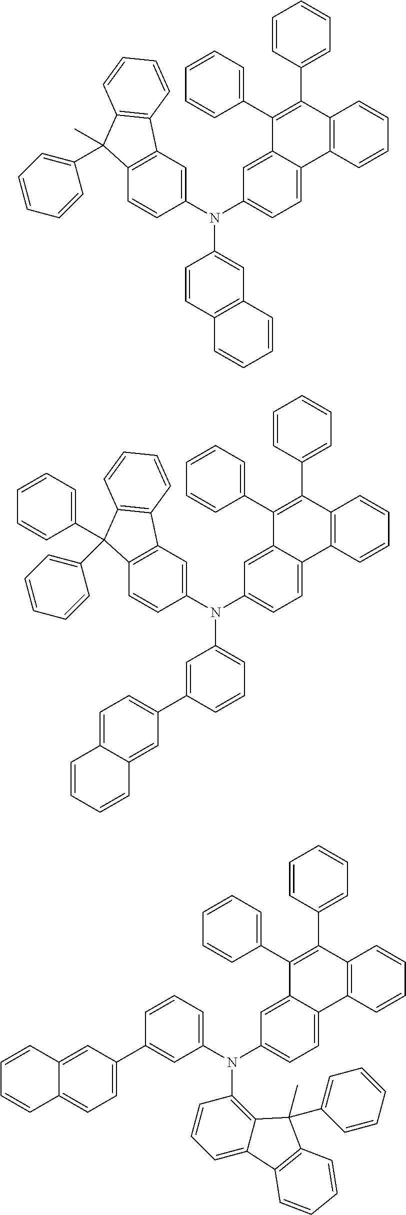 [SMILES: CC1(c2ccccc2)c2ccccc2-c2cc(N(c3ccc4ccccc4c3)c3ccc4c(c3)c(-c3ccccc3)c(-c3ccccc3)c3ccccc34)ccc21 ; c1ccc(-c2c(-c3ccccc3)c3cc(N(c4cccc(-c5ccc6ccccc6c5)c4)c4ccc5c(c4)-c4ccccc4C5(c4ccccc4)c4ccccc4)ccc3c3ccccc23)cc1 ; CC1(c2ccccc2)c2ccccc2-c2cccc(N(c3cccc(-c4ccc5ccccc5c4)c3)c3ccc4c(c3)c(-c3ccccc3)c(-c3ccccc3)c3ccccc34)c21]

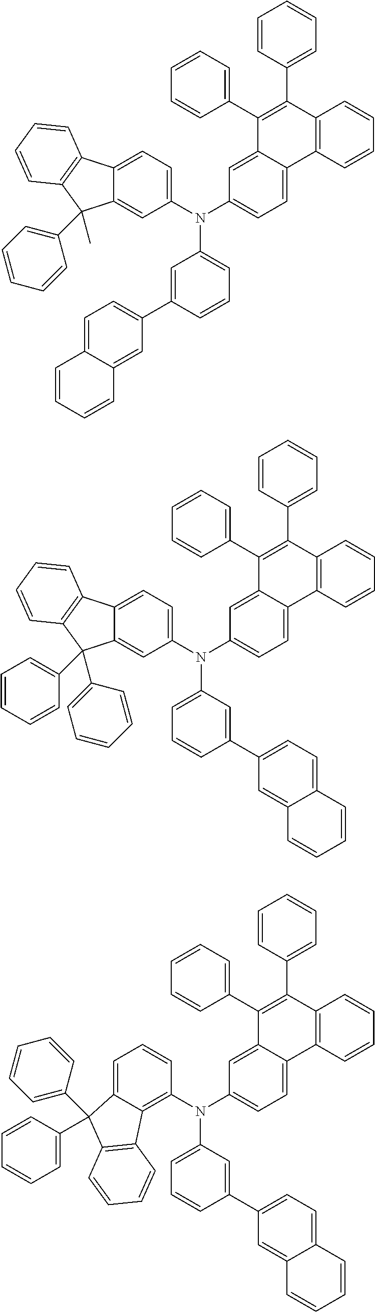 [SMILES: CC1(c2ccccc2)c2ccccc2-c2ccc(N(c3cccc(-c4ccc5ccccc5c4)c3)c3ccc4c(c3)c(-c3ccccc3)c(-c3ccccc3)c3ccccc34)cc21 ; c1ccc(-c2c(-c3ccccc3)c3cc(N(c4cccc(-c5ccc6ccccc6c5)c4)c4ccc5c(c4)C(c4ccccc4)(c4ccccc4)c4ccccc4-5)ccc3c3ccccc23)cc1 ; c1ccc(-c2c(-c3ccccc3)c3cc(N(c4cccc(-c5ccc6ccccc6c5)c4)c4cccc5c4-c4ccccc4C5(c4ccccc4)c4ccccc4)ccc3c3ccccc23)cc1]

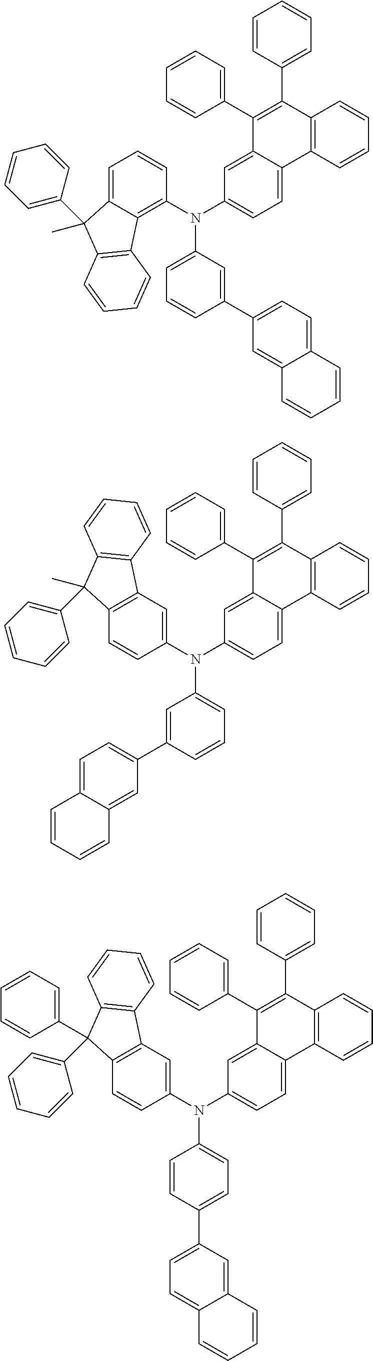

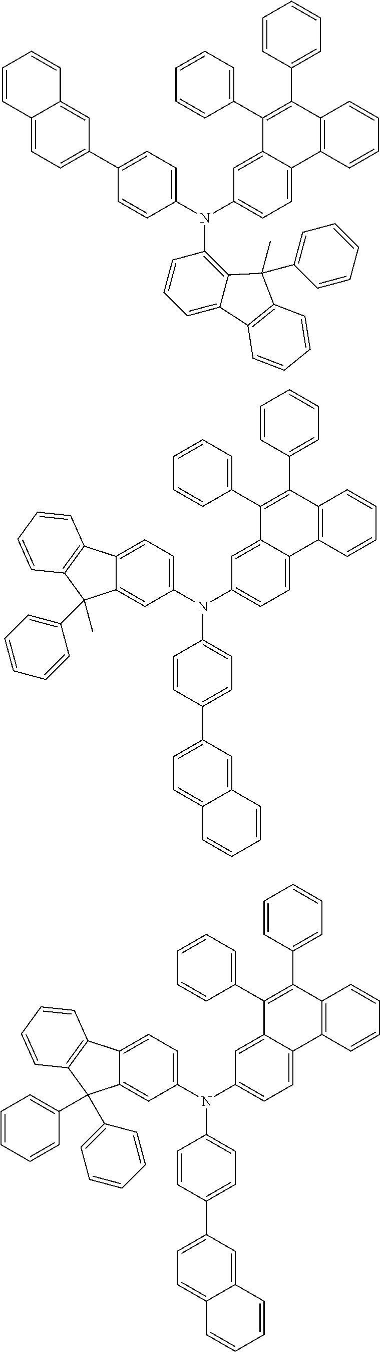

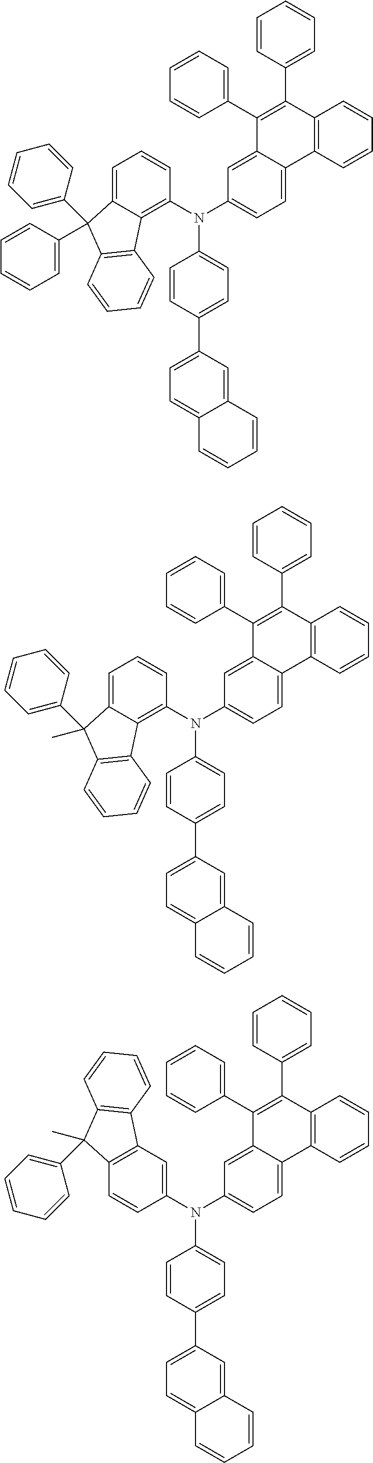 [SMILES: c1ccc(-c2c(-c3ccccc3)c3cc(N(c4ccc(-c5ccc6ccccc6c5)cc4)c4cccc5c4-c4ccccc4C5(c4ccccc4)c4ccccc4)ccc3c3ccccc23)cc1 ; CC1(c2ccccc2)c2ccccc2-c2c(N(c3ccc(-c4ccc5ccccc5c4)cc3)c3ccc4c(c3)c(-c3ccccc3)c(-c3ccccc3)c3ccccc34)cccc21 ; CC1(c2ccccc2)c2ccccc2-c2cc(N(c3ccc(-c4ccc5ccccc5c4)cc3)c3ccc4c(c3)c(-c3ccccc3)c(-c3ccccc3)c3ccccc34)ccc21]

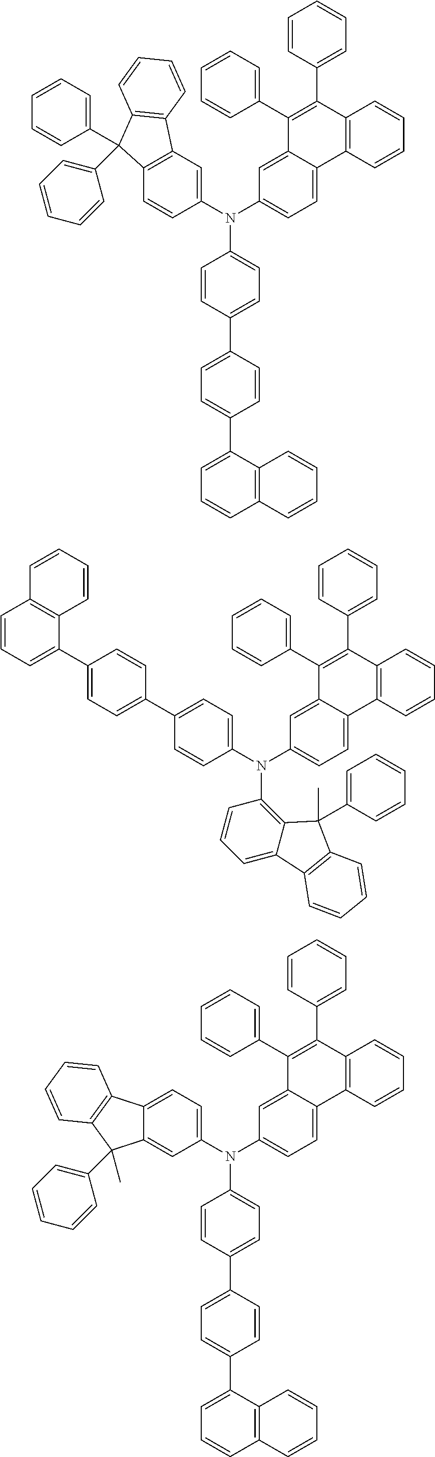

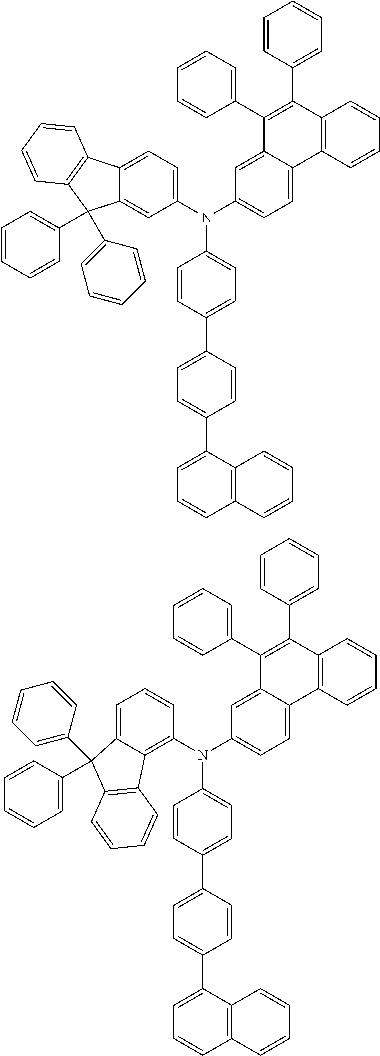

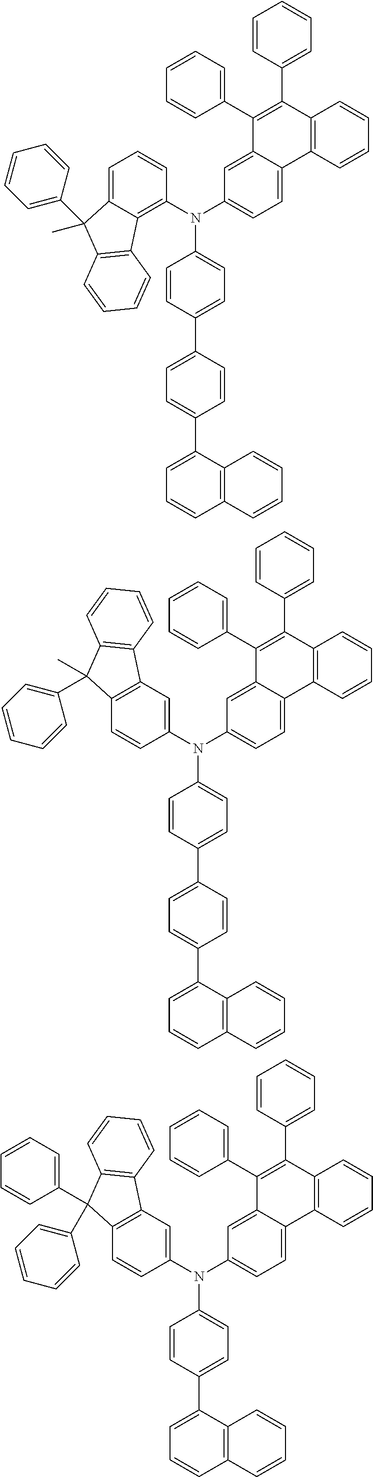

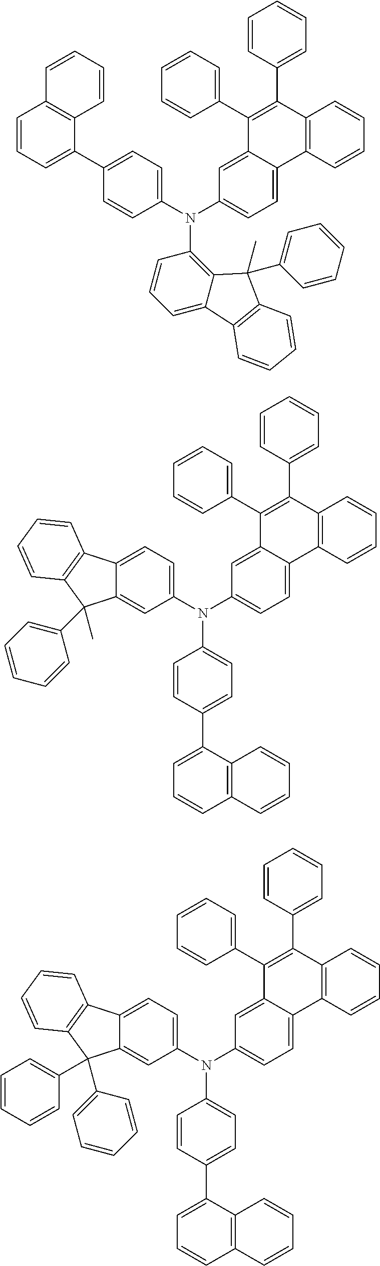

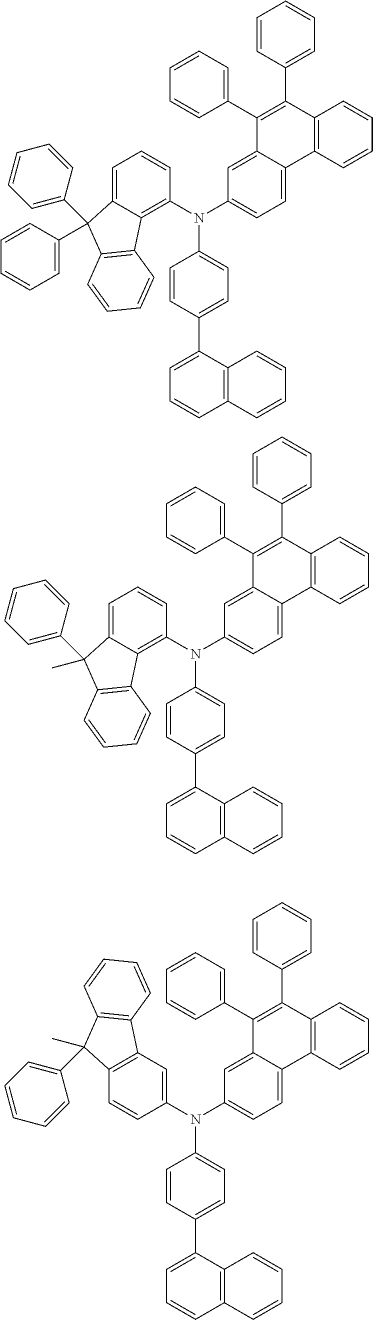

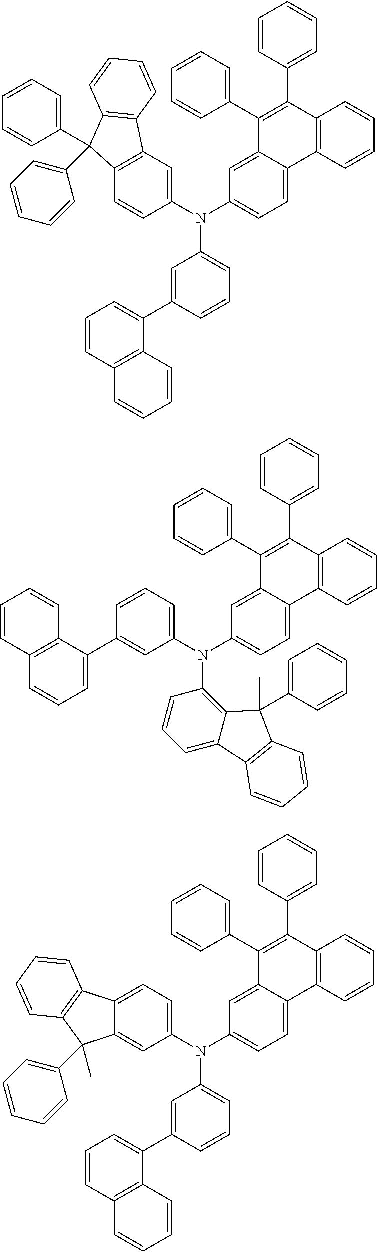

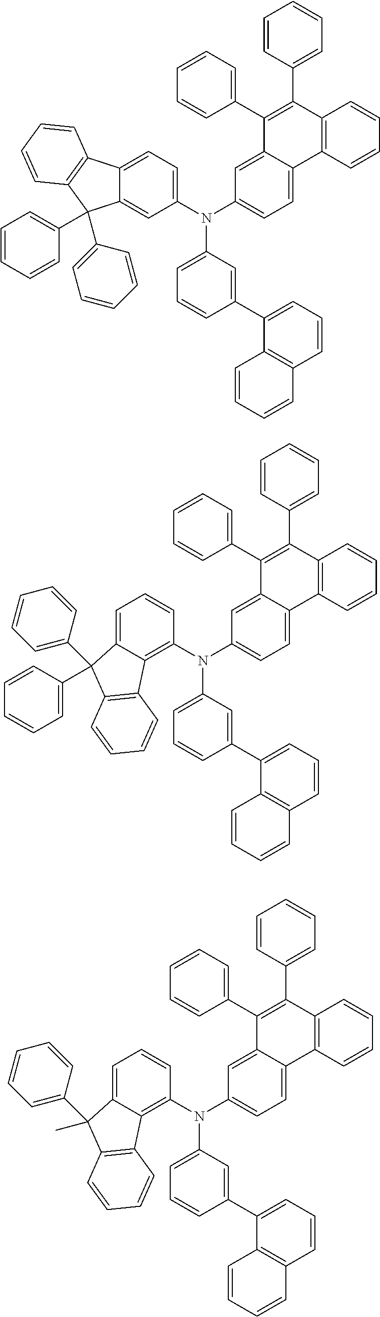

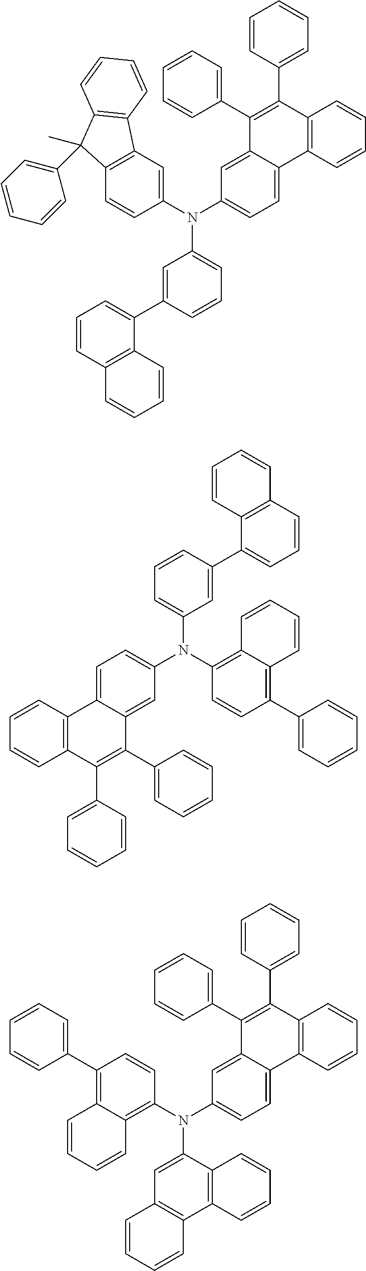 [SMILES: CC1(c2ccccc2)c2ccccc2-c2cc(N(c3cccc(-c4cccc5ccccc45)c3)c3ccc4c(c3)c(-c3ccccc3)c(-c3ccccc3)c3ccccc34)ccc21 ; c1ccc(-c2ccc(N(c3cccc(-c4cccc5ccccc45)c3)c3ccc4c(c3)c(-c3ccccc3)c(-c3ccccc3)c3ccccc34)c3ccccc23)cc1 ; c1ccc(-c2ccc(N(c3ccc4c(c3)c(-c3ccccc3)c(-c3ccccc3)c3ccccc34)c3cc4ccccc4c4ccccc34)c3ccccc23)cc1]

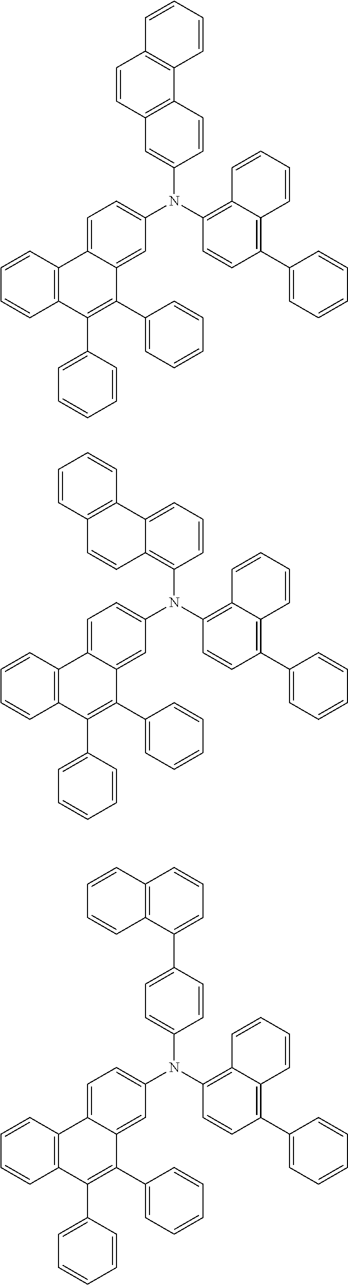

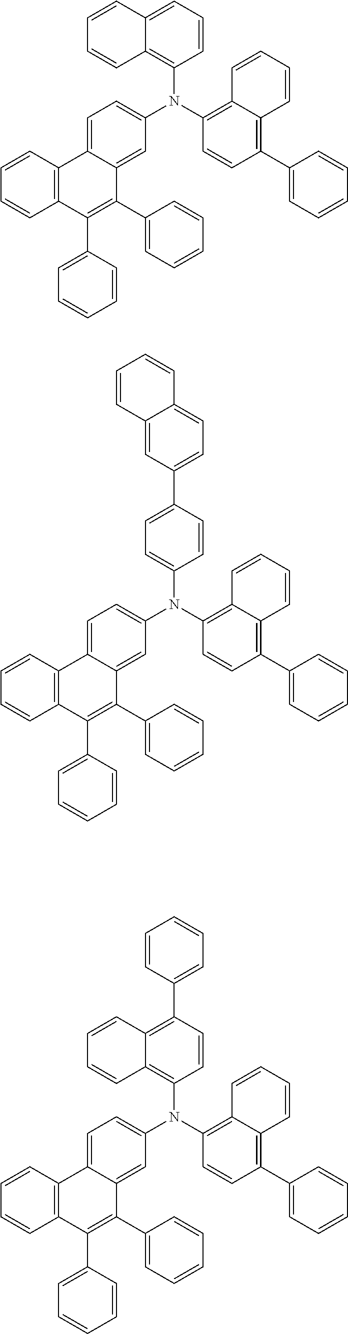 [SMILES: c1ccc(-c2ccc(N(c3ccc4c(c3)c(-c3ccccc3)c(-c3ccccc3)c3ccccc34)c3cccc4ccccc34)c3ccccc23)cc1 ; c1ccc(-c2ccc(N(c3ccc(-c4ccc5ccccc5c4)cc3)c3ccc4c(c3)c(-c3ccccc3)c(-c3ccccc3)c3ccccc34)c3ccccc23)cc1 ; c1ccc(-c2ccc(N(c3ccc4c(c3)c(-c3ccccc3)c(-c3ccccc3)c3ccccc34)c3ccc(-c4ccccc4)c4ccccc34)c3ccccc23)cc1]

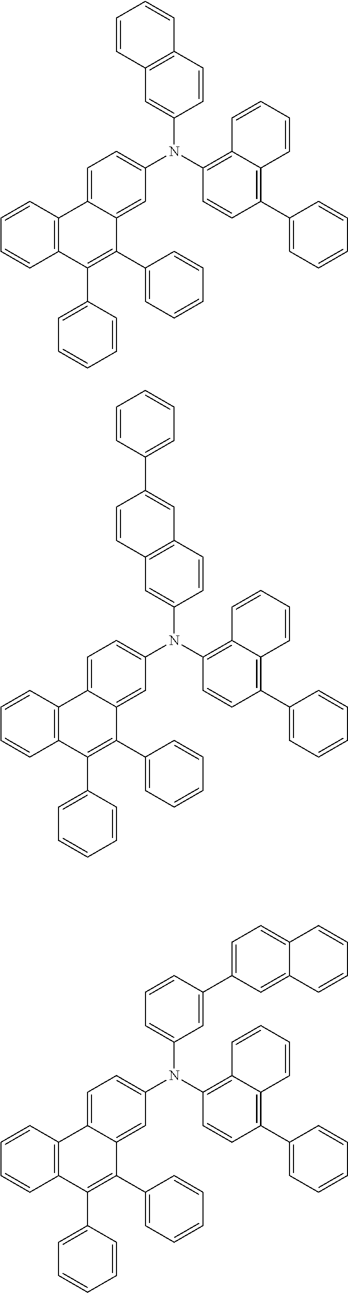 [SMILES: c1ccc(-c2ccc(N(c3ccc4ccccc4c3)c3ccc4c(c3)c(-c3ccccc3)c(-c3ccccc3)c3ccccc34)c3ccccc23)cc1 ; c1ccc(-c2ccc3cc(N(c4ccc5c(c4)c(-c4ccccc4)c(-c4ccccc4)c4ccccc45)c4ccc(-c5ccccc5)c5ccccc45)ccc3c2)cc1 ; c1ccc(-c2ccc(N(c3cccc(-c4ccc5ccccc5c4)c3)c3ccc4c(c3)c(-c3ccccc3)c(-c3ccccc3)c3ccccc34)c3ccccc23)cc1]

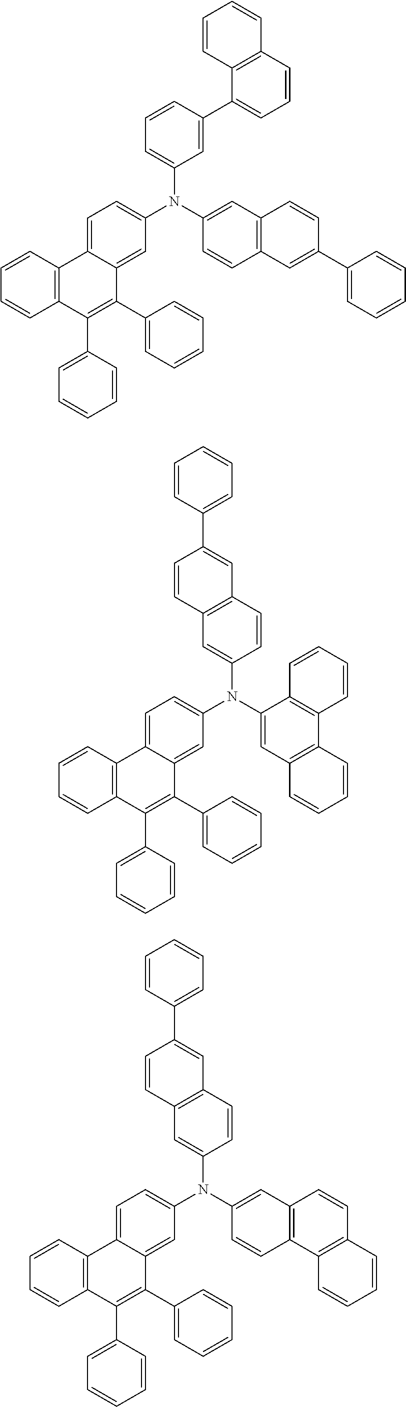

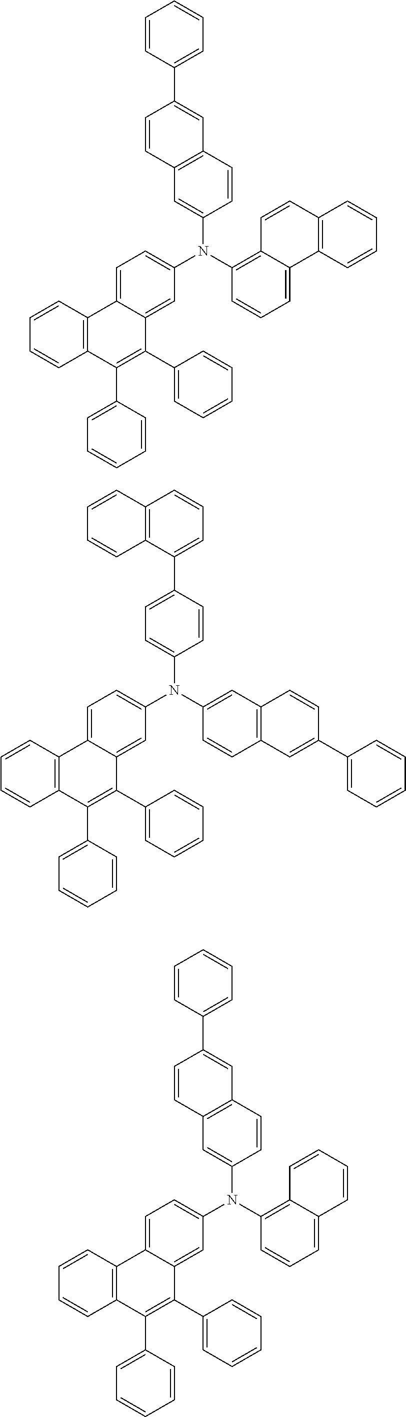

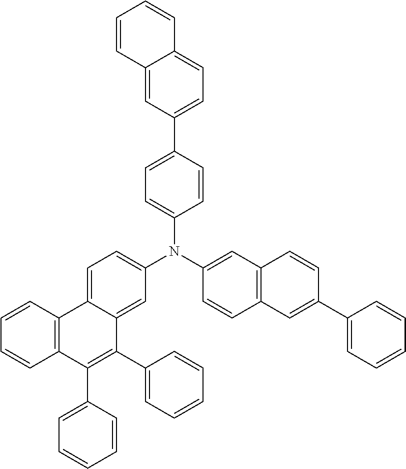 [SMILES: c1ccc(-c2ccc3cc(N(c4ccc(-c5ccc6ccccc6c5)cc4)c4ccc5c(c4)c(-c4ccccc4)c(-c4ccccc4)c4ccccc45)ccc3c2)cc1]

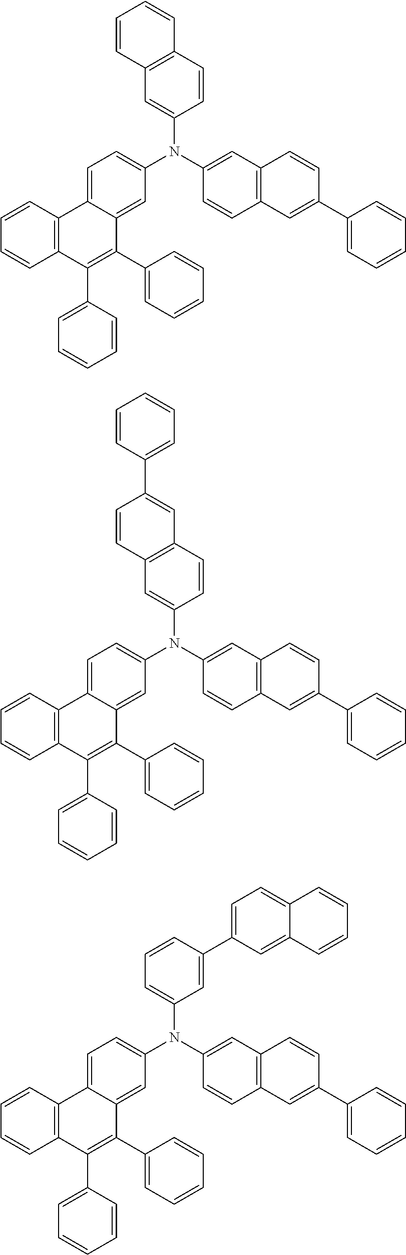 [SMILES: c1ccc(-c2ccc3cc(N(c4ccc5ccccc5c4)c4ccc5c(c4)c(-c4ccccc4)c(-c4ccccc4)c4ccccc45)ccc3c2)cc1 ; c1ccc(-c2ccc3cc(N(c4ccc5cc(-c6ccccc6)ccc5c4)c4ccc5c(c4)c(-c4ccccc4)c(-c4ccccc4)c4ccccc45)ccc3c2)cc1 ; c1ccc(-c2ccc3cc(N(c4cccc(-c5ccc6ccccc6c5)c4)c4ccc5c(c4)c(-c4ccccc4)c(-c4ccccc4)c4ccccc45)ccc3c2)cc1]

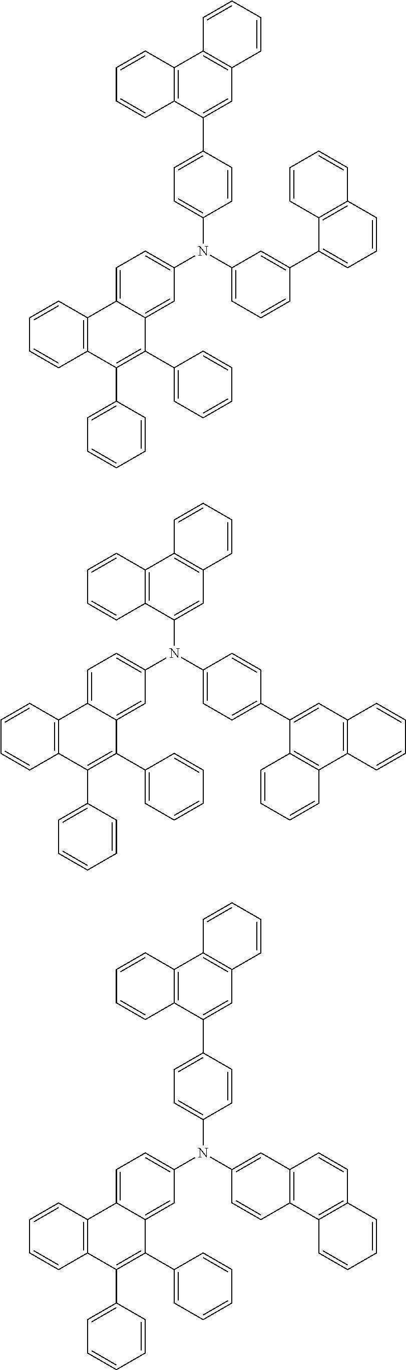 [SMILES: c1ccc(-c2c(-c3ccccc3)c3cc(N(c4ccc(-c5cc6ccccc6c6ccccc56)cc4)c4cccc(-c5cccc6ccccc56)c4)ccc3c3ccccc23)cc1 ; c1ccc(-c2c(-c3ccccc3)c3cc(N(c4ccc(-c5cc6ccccc6c6ccccc56)cc4)c4cc5ccccc5c5ccccc45)ccc3c3ccccc23)cc1 ; c1ccc(-c2c(-c3ccccc3)c3cc(N(c4ccc(-c5cc6ccccc6c6ccccc56)cc4)c4ccc5c(ccc6ccccc65)c4)ccc3c3ccccc23)cc1]

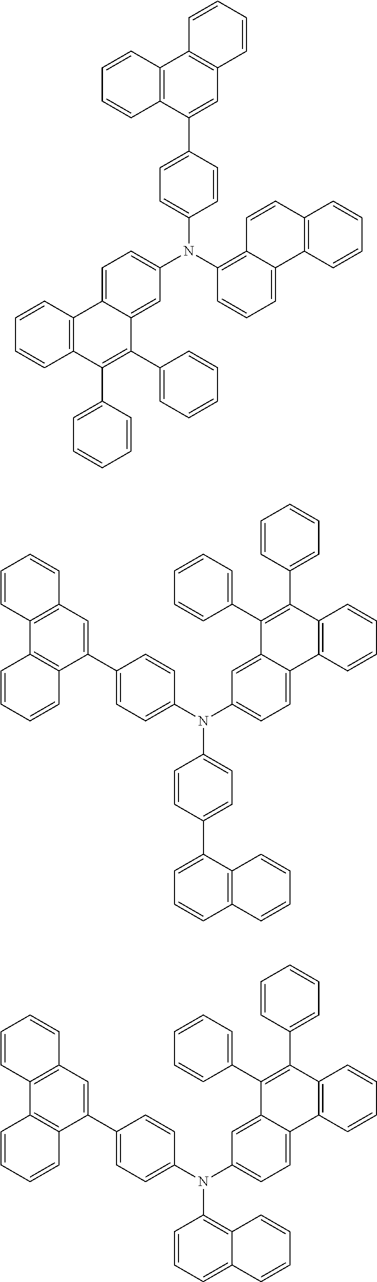 [SMILES: c1ccc(-c2c(-c3ccccc3)c3cc(N(c4ccc(-c5cc6ccccc6c6ccccc56)cc4)c4cccc5c4ccc4ccccc45)ccc3c3ccccc23)cc1 ; c1ccc(-c2c(-c3ccccc3)c3cc(N(c4ccc(-c5cccc6ccccc56)cc4)c4ccc(-c5cc6ccccc6c6ccccc56)cc4)ccc3c3ccccc23)cc1 ; c1ccc(-c2c(-c3ccccc3)c3cc(N(c4ccc(-c5cc6ccccc6c6ccccc56)cc4)c4cccc5ccccc45)ccc3c3ccccc23)cc1]

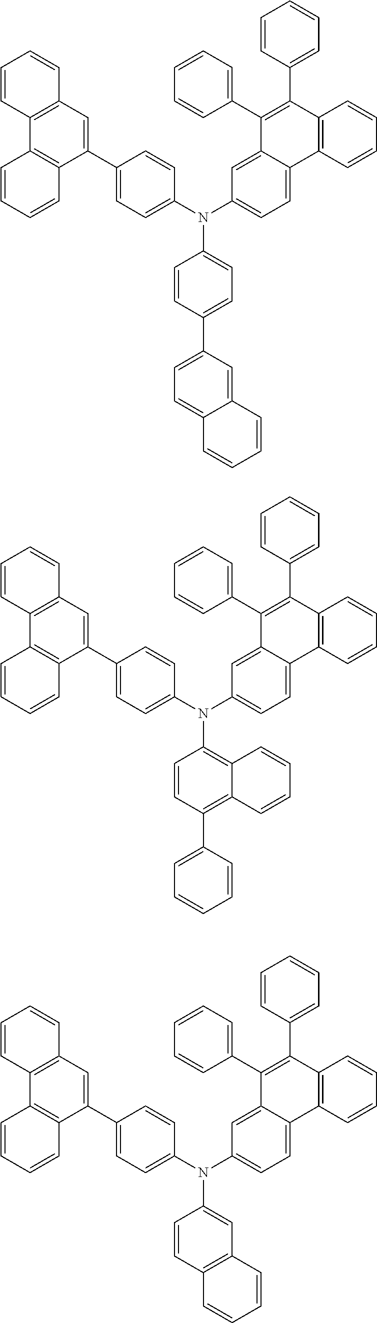 [SMILES: c1ccc(-c2c(-c3ccccc3)c3cc(N(c4ccc(-c5ccc6ccccc6c5)cc4)c4ccc(-c5cc6ccccc6c6ccccc56)cc4)ccc3c3ccccc23)cc1 ; c1ccc(-c2ccc(N(c3ccc(-c4cc5ccccc5c5ccccc45)cc3)c3ccc4c(c3)c(-c3ccccc3)c(-c3ccccc3)c3ccccc34)c3ccccc23)cc1 ; c1ccc(-c2c(-c3ccccc3)c3cc(N(c4ccc(-c5cc6ccccc6c6ccccc56)cc4)c4ccc5ccccc5c4)ccc3c3ccccc23)cc1]

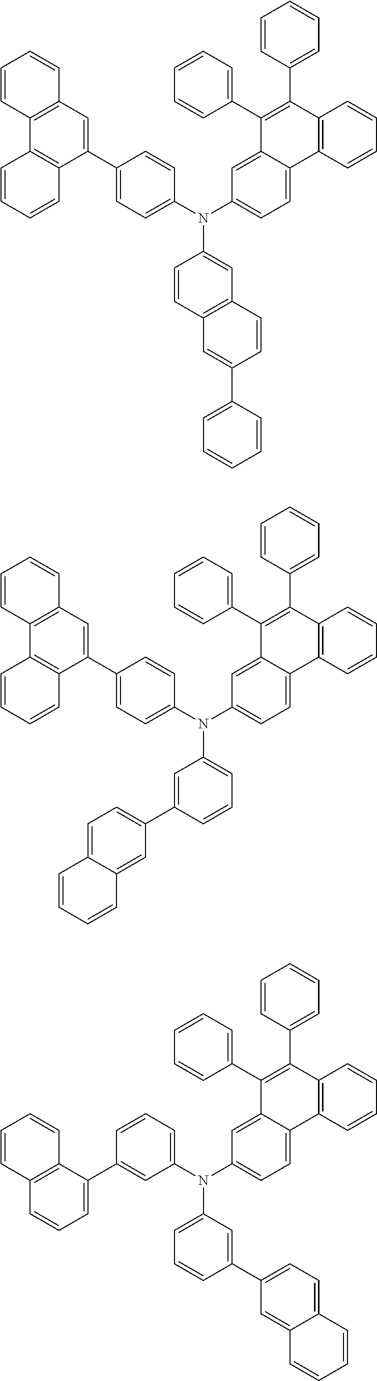 [SMILES: c1ccc(-c2ccc3cc(N(c4ccc(-c5cc6ccccc6c6ccccc56)cc4)c4ccc5c(c4)c(-c4ccccc4)c(-c4ccccc4)c4ccccc45)ccc3c2)cc1 ; c1ccc(-c2c(-c3ccccc3)c3cc(N(c4ccc(-c5cc6ccccc6c6ccccc56)cc4)c4cccc(-c5ccc6ccccc6c5)c4)ccc3c3ccccc23)cc1 ; c1ccc(-c2c(-c3ccccc3)c3cc(N(c4cccc(-c5ccc6ccccc6c5)c4)c4cccc(-c5cccc6ccccc56)c4)ccc3c3ccccc23)cc1]

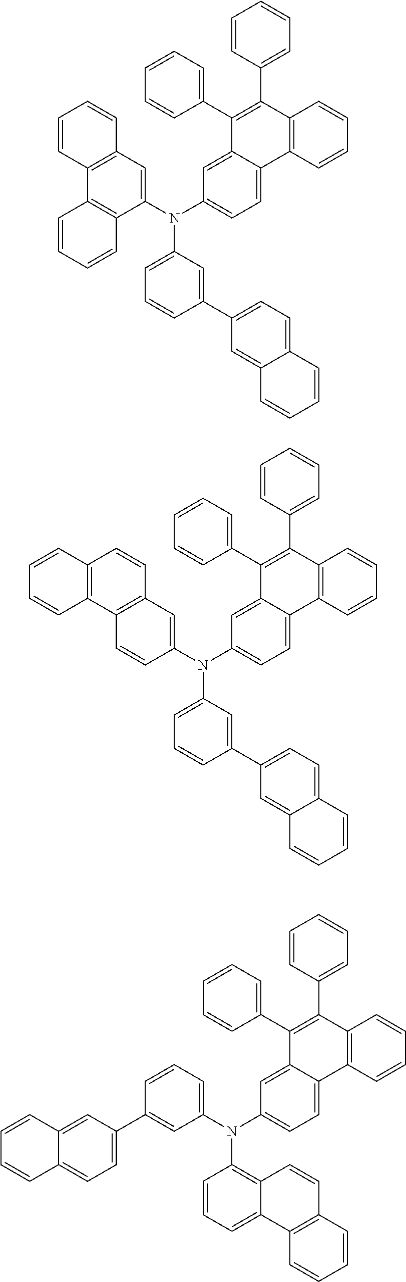

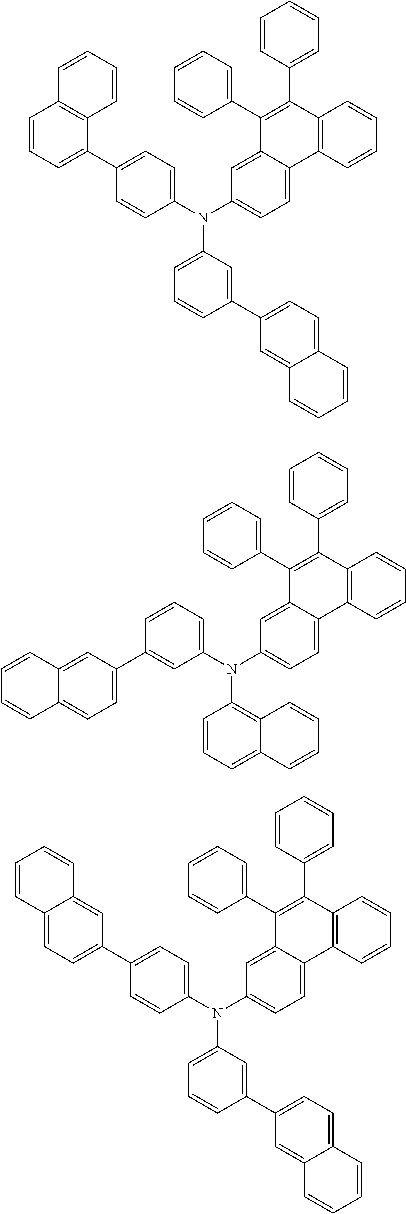

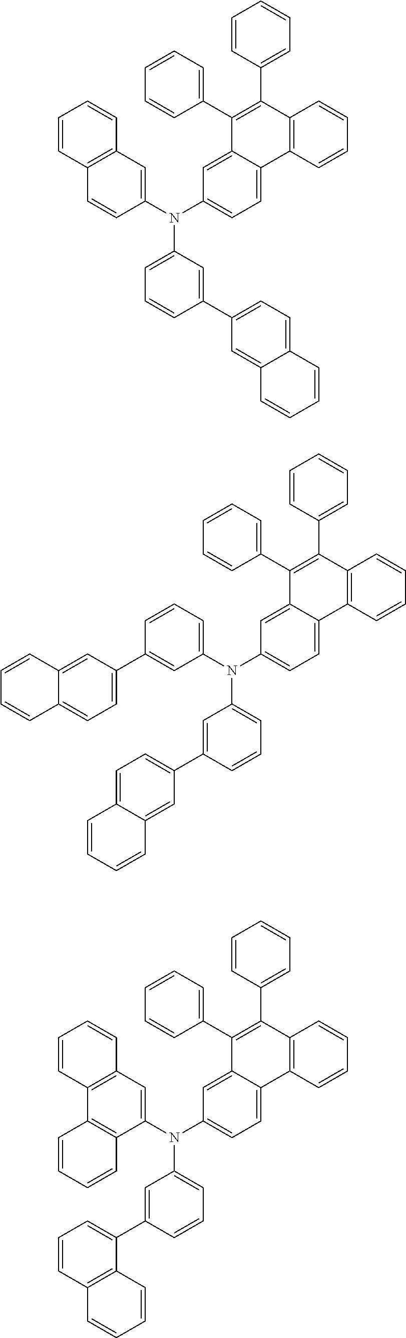

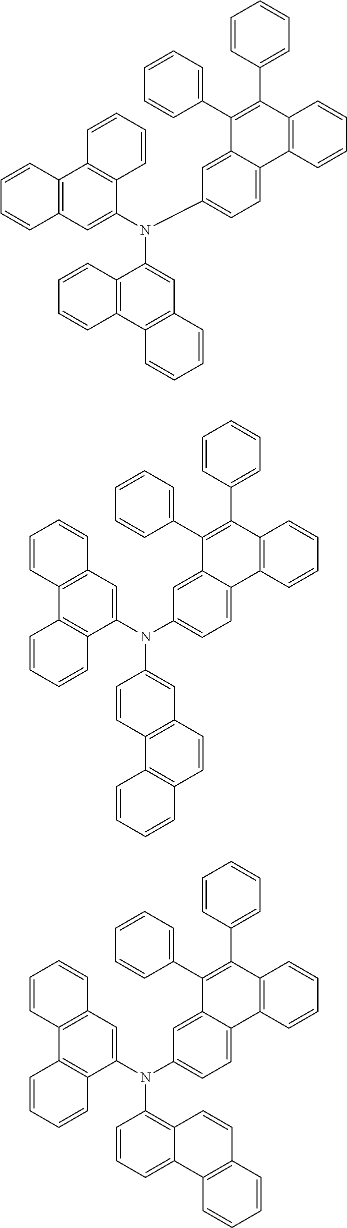 [SMILES: c1ccc(-c2c(-c3ccccc3)c3cc(N(c4cc5ccccc5c5ccccc45)c4cc5ccccc5c5ccccc45)ccc3c3ccccc23)cc1 ; c1ccc(-c2c(-c3ccccc3)c3cc(N(c4ccc5c(ccc6ccccc65)c4)c4cc5ccccc5c5ccccc45)ccc3c3ccccc23)cc1 ; c1ccc(-c2c(-c3ccccc3)c3cc(N(c4cccc5c4ccc4ccccc45)c4cc5ccccc5c5ccccc45)ccc3c3ccccc23)cc1]

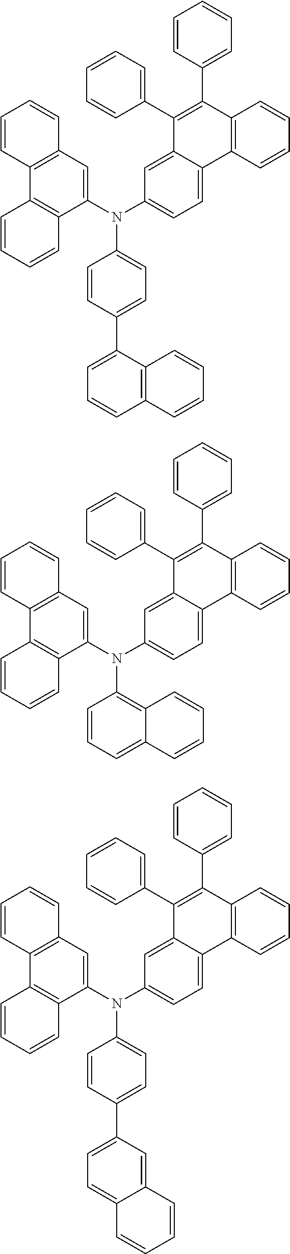

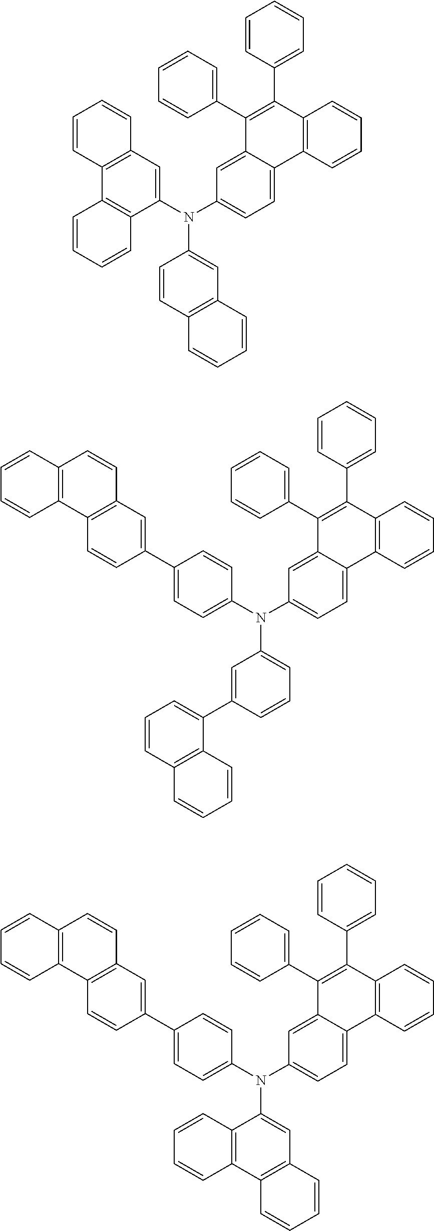 [SMILES: c1ccc(-c2c(-c3ccccc3)c3cc(N(c4ccc5ccccc5c4)c4cc5ccccc5c5ccccc45)ccc3c3ccccc23)cc1 ; c1ccc(-c2c(-c3ccccc3)c3cc(N(c4ccc(-c5ccc6c(ccc7ccccc76)c5)cc4)c4cccc(-c5cccc6ccccc56)c4)ccc3c3ccccc23)cc1 ; c1ccc(-c2c(-c3ccccc3)c3cc(N(c4ccc(-c5ccc6c(ccc7ccccc76)c5)cc4)c4cc5ccccc5c5ccccc45)ccc3c3ccccc23)cc1]

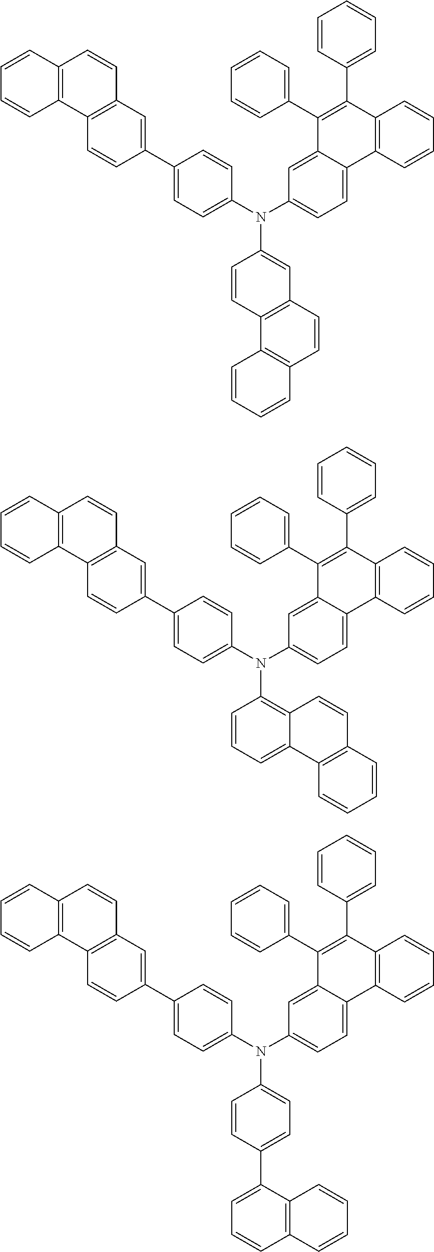

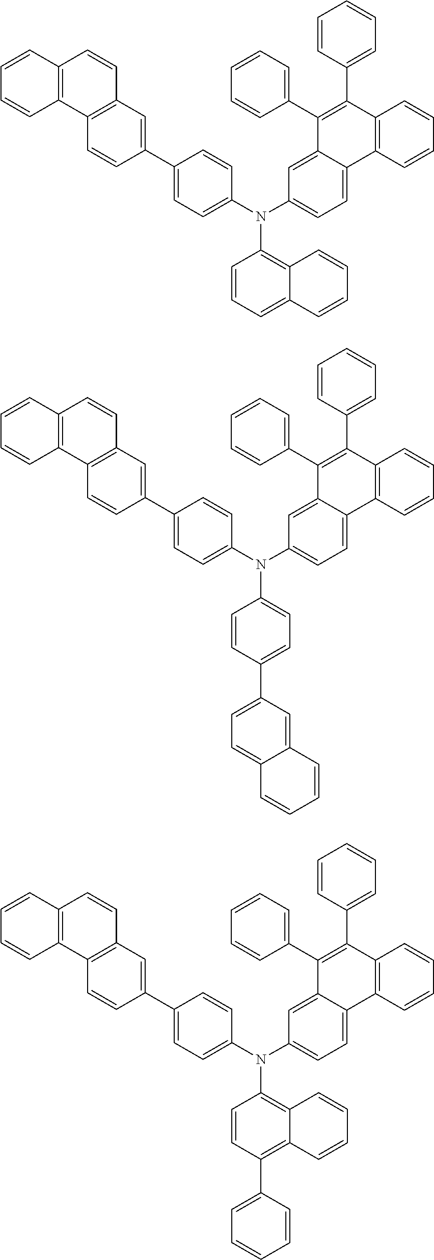

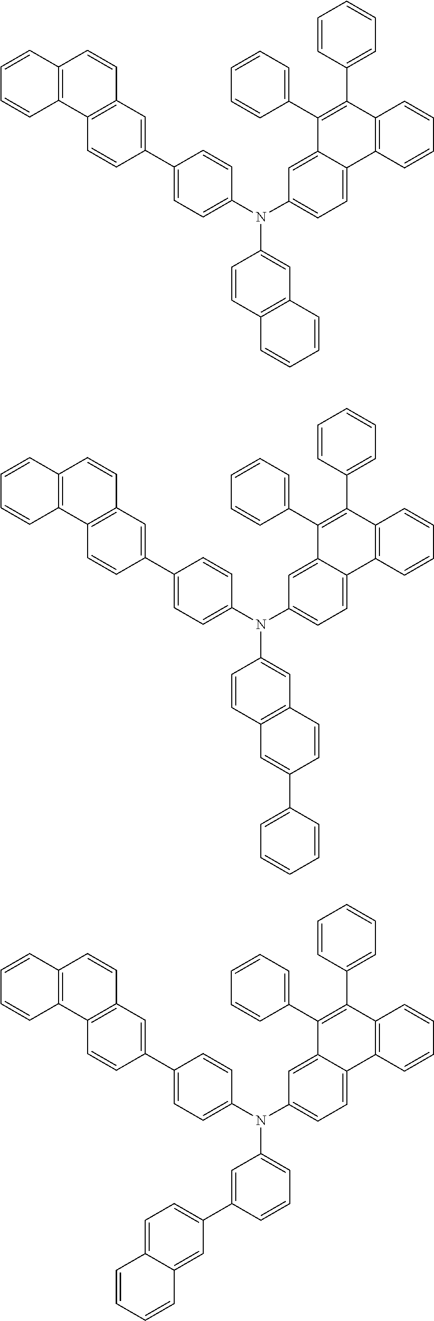 [SMILES: c1ccc(-c2c(-c3ccccc3)c3cc(N(c4ccc(-c5ccc6c(ccc7ccccc76)c5)cc4)c4ccc5ccccc5c4)ccc3c3ccccc23)cc1 ; c1ccc(-c2ccc3cc(N(c4ccc(-c5ccc6c(ccc7ccccc76)c5)cc4)c4ccc5c(c4)c(-c4ccccc4)c(-c4ccccc4)c4ccccc45)ccc3c2)cc1 ; c1ccc(-c2c(-c3ccccc3)c3cc(N(c4ccc(-c5ccc6c(ccc7ccccc76)c5)cc4)c4cccc(-c5ccc6ccccc6c5)c4)ccc3c3ccccc23)cc1]

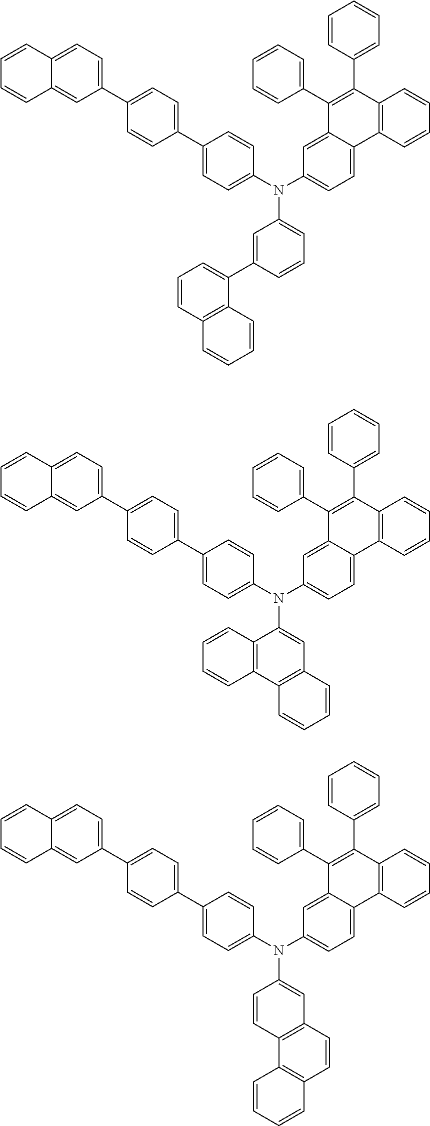 [SMILES: c1ccc(-c2c(-c3ccccc3)c3cc(N(c4ccc(-c5ccc(-c6ccc7ccccc7c6)cc5)cc4)c4cccc(-c5cccc6ccccc56)c4)ccc3c3ccccc23)cc1 ; c1ccc(-c2c(-c3ccccc3)c3cc(N(c4ccc(-c5ccc(-c6ccc7ccccc7c6)cc5)cc4)c4cc5ccccc5c5ccccc45)ccc3c3ccccc23)cc1 ; c1ccc(-c2c(-c3ccccc3)c3cc(N(c4ccc(-c5ccc(-c6ccc7ccccc7c6)cc5)cc4)c4ccc5c(ccc6ccccc65)c4)ccc3c3ccccc23)cc1]

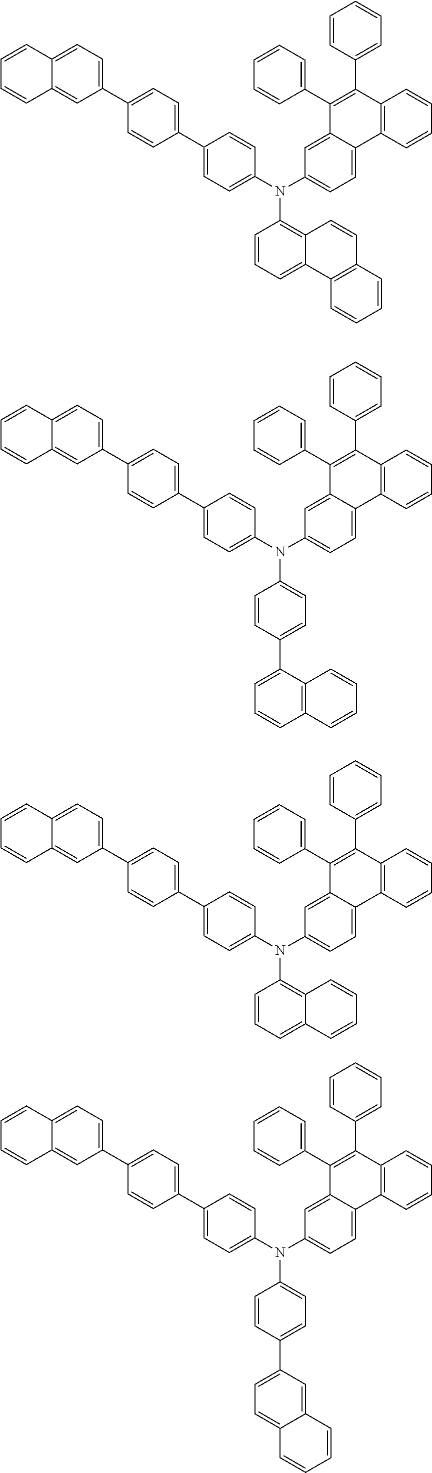

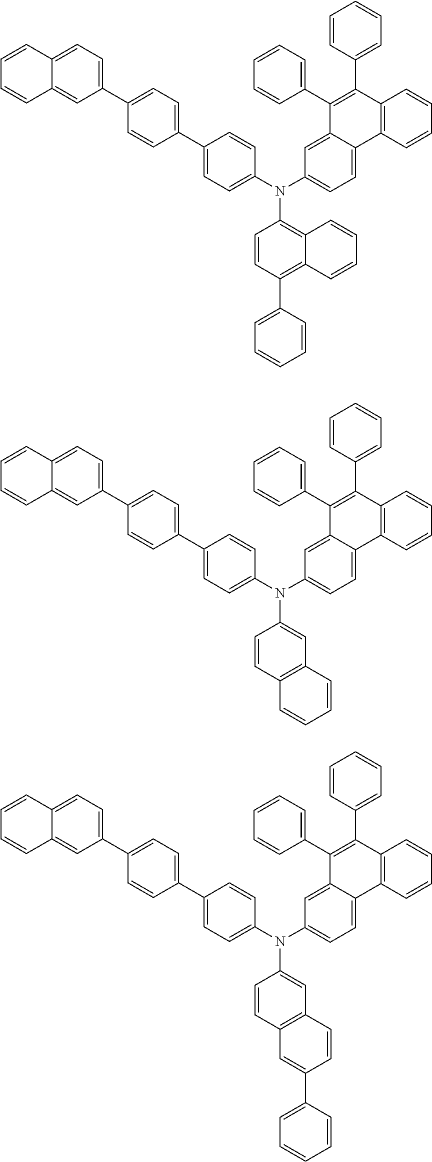 [SMILES: c1ccc(-c2ccc(N(c3ccc(-c4ccc(-c5ccc6ccccc6c5)cc4)cc3)c3ccc4c(c3)c(-c3ccccc3)c(-c3ccccc3)c3ccccc34)c3ccccc23)cc1 ; c1ccc(-c2c(-c3ccccc3)c3cc(N(c4ccc(-c5ccc(-c6ccc7ccccc7c6)cc5)cc4)c4ccc5ccccc5c4)ccc3c3ccccc23)cc1 ; c1ccc(-c2ccc3cc(N(c4ccc(-c5ccc(-c6ccc7ccccc7c6)cc5)cc4)c4ccc5c(c4)c(-c4ccccc4)c(-c4ccccc4)c4ccccc45)ccc3c2)cc1]

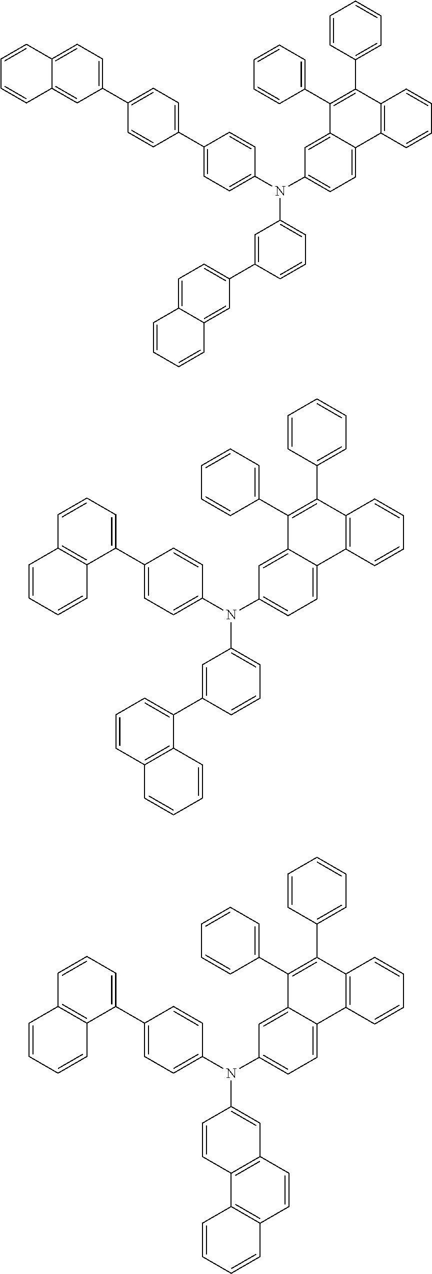 [SMILES: c1ccc(-c2c(-c3ccccc3)c3cc(N(c4ccc(-c5ccc(-c6ccc7ccccc7c6)cc5)cc4)c4cccc(-c5ccc6ccccc6c5)c4)ccc3c3ccccc23)cc1 ; c1ccc(-c2c(-c3ccccc3)c3cc(N(c4ccc(-c5cccc6ccccc56)cc4)c4cccc(-c5cccc6ccccc56)c4)ccc3c3ccccc23)cc1 ; c1ccc(-c2c(-c3ccccc3)c3cc(N(c4ccc(-c5cccc6ccccc56)cc4)c4ccc5c(ccc6ccccc65)c4)ccc3c3ccccc23)cc1]

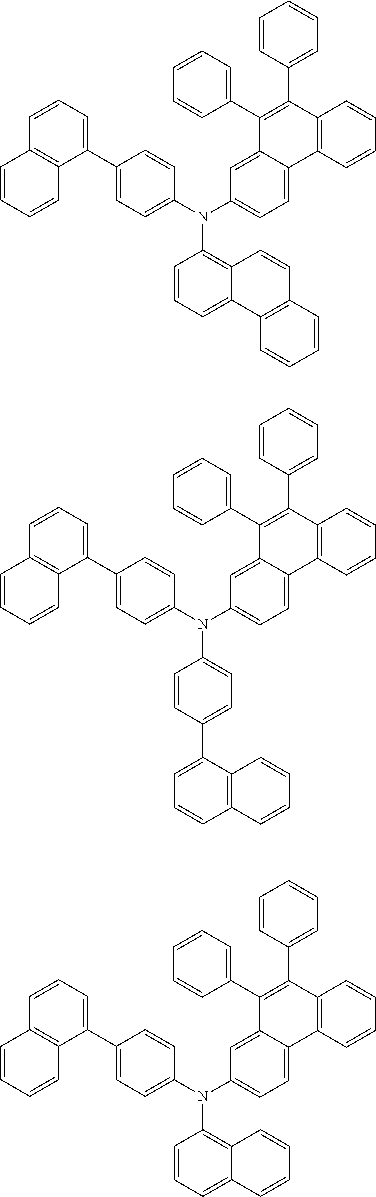

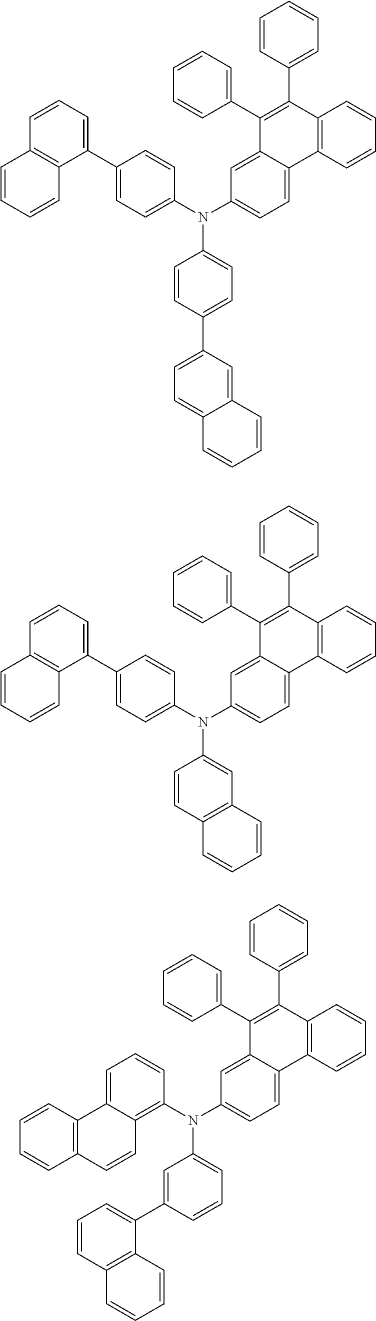 [SMILES: c1ccc(-c2c(-c3ccccc3)c3cc(N(c4ccc(-c5ccc6ccccc6c5)cc4)c4ccc(-c5cccc6ccccc56)cc4)ccc3c3ccccc23)cc1 ; c1ccc(-c2c(-c3ccccc3)c3cc(N(c4ccc(-c5cccc6ccccc56)cc4)c4ccc5ccccc5c4)ccc3c3ccccc23)cc1 ; c1ccc(-c2c(-c3ccccc3)c3cc(N(c4cccc(-c5cccc6ccccc56)c4)c4cccc5c4ccc4ccccc45)ccc3c3ccccc23)cc1]

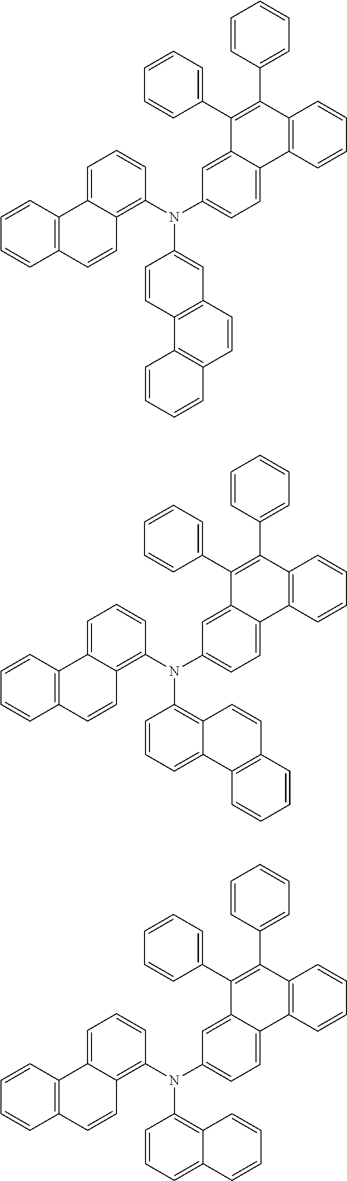

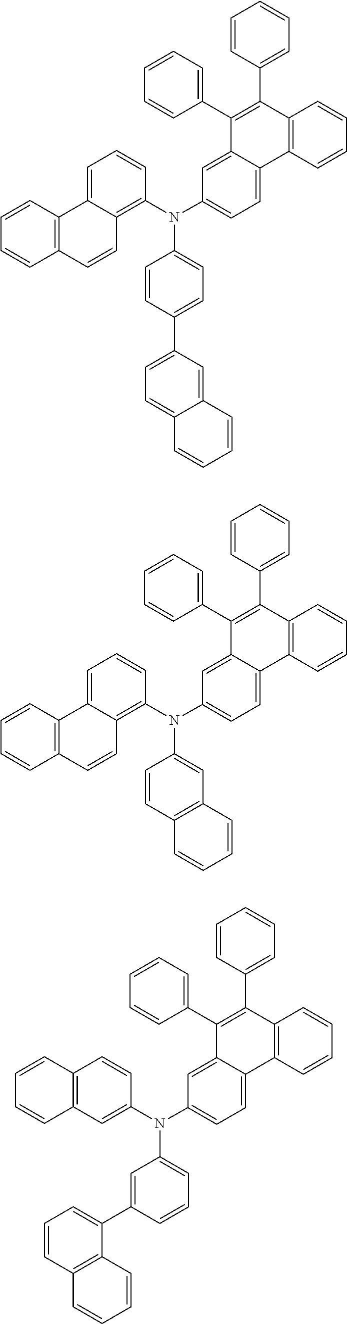 [SMILES: c1ccc(-c2c(-c3ccccc3)c3cc(N(c4ccc(-c5ccc6ccccc6c5)cc4)c4cccc5c4ccc4ccccc45)ccc3c3ccccc23)cc1 ; c1ccc(-c2c(-c3ccccc3)c3cc(N(c4ccc5ccccc5c4)c4cccc5c4ccc4ccccc45)ccc3c3ccccc23)cc1 ; c1ccc(-c2c(-c3ccccc3)c3cc(N(c4cccc(-c5cccc6ccccc56)c4)c4ccc5ccccc5c4)ccc3c3ccccc23)cc1]

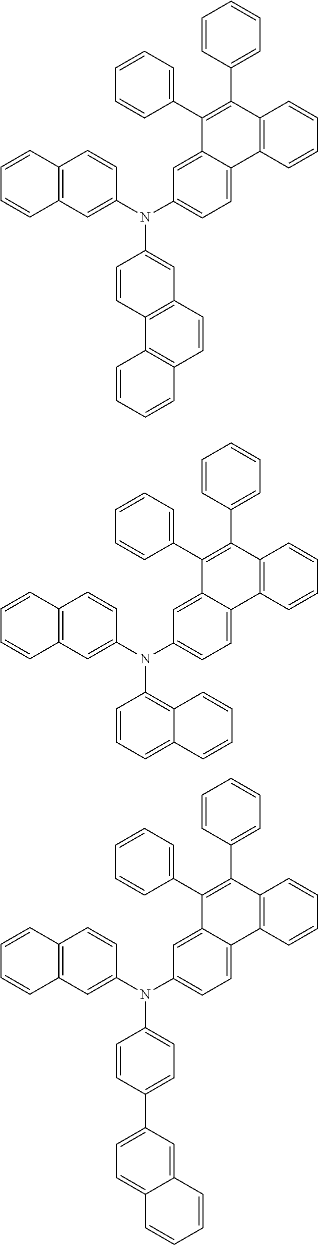

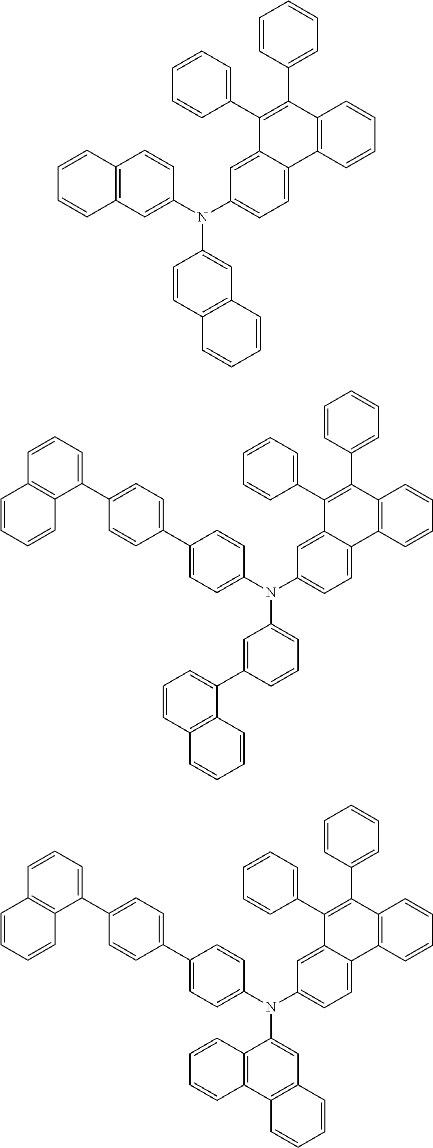 [SMILES: c1ccc(-c2c(-c3ccccc3)c3cc(N(c4ccc5ccccc5c4)c4ccc5ccccc5c4)ccc3c3ccccc23)cc1 ; c1ccc(-c2c(-c3ccccc3)c3cc(N(c4ccc(-c5ccc(-c6cccc7ccccc67)cc5)cc4)c4cccc(-c5cccc6ccccc56)c4)ccc3c3ccccc23)cc1 ; c1ccc(-c2c(-c3ccccc3)c3cc(N(c4ccc(-c5ccc(-c6cccc7ccccc67)cc5)cc4)c4cc5ccccc5c5ccccc45)ccc3c3ccccc23)cc1]

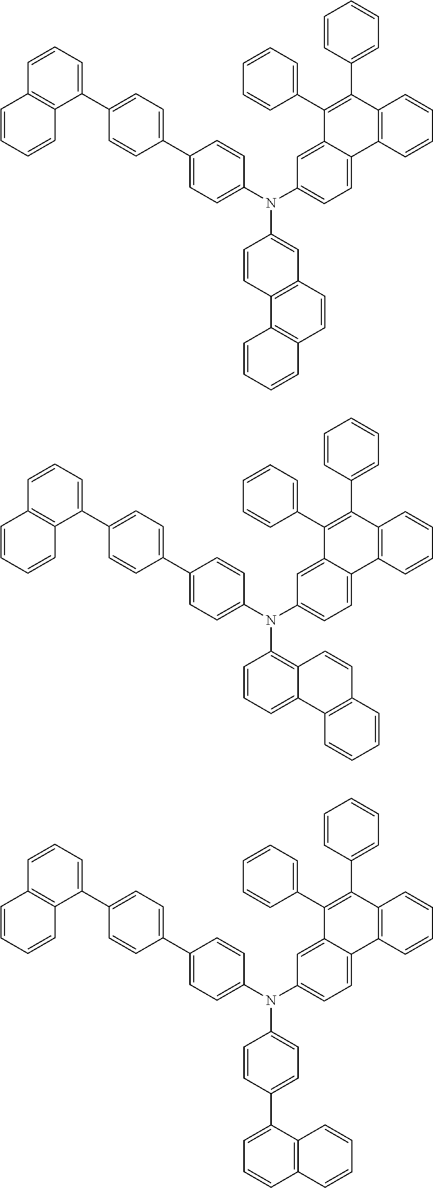

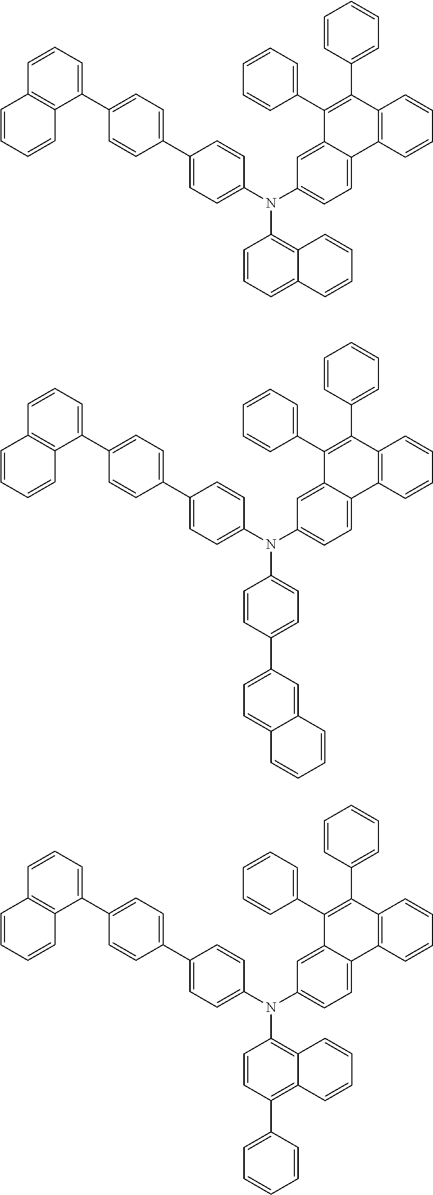 [SMILES: c1ccc(-c2c(-c3ccccc3)c3cc(N(c4ccc(-c5ccc(-c6cccc7ccccc67)cc5)cc4)c4cccc5ccccc45)ccc3c3ccccc23)cc1 ; c1ccc(-c2c(-c3ccccc3)c3cc(N(c4ccc(-c5ccc(-c6cccc7ccccc67)cc5)cc4)c4ccc(-c5ccc6ccccc6c5)cc4)ccc3c3ccccc23)cc1 ; c1ccc(-c2ccc(N(c3ccc(-c4ccc(-c5cccc6ccccc56)cc4)cc3)c3ccc4c(c3)c(-c3ccccc3)c(-c3ccccc3)c3ccccc34)c3ccccc23)cc1]

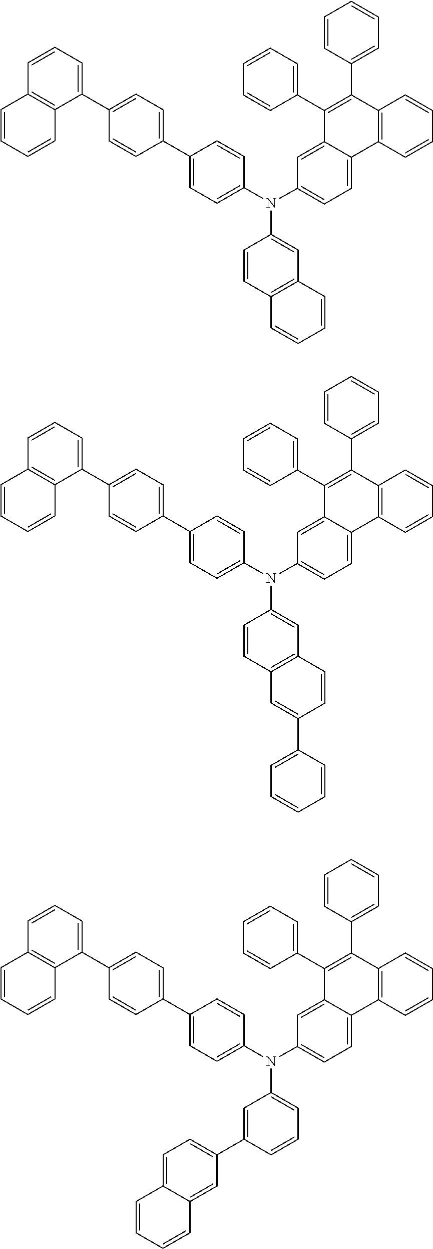 [SMILES: c1ccc(-c2c(-c3ccccc3)c3cc(N(c4ccc(-c5ccc(-c6cccc7ccccc67)cc5)cc4)c4ccc5ccccc5c4)ccc3c3ccccc23)cc1 ; c1ccc(-c2ccc3cc(N(c4ccc(-c5ccc(-c6cccc7ccccc67)cc5)cc4)c4ccc5c(c4)c(-c4ccccc4)c(-c4ccccc4)c4ccccc45)ccc3c2)cc1 ; c1ccc(-c2c(-c3ccccc3)c3cc(N(c4ccc(-c5ccc(-c6cccc7ccccc67)cc5)cc4)c4cccc(-c5ccc6ccccc6c5)c4)ccc3c3ccccc23)cc1]

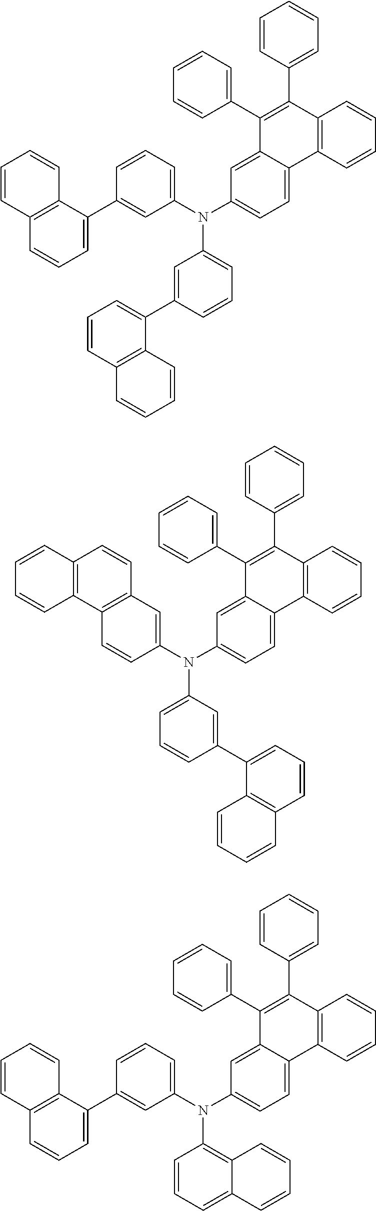 [SMILES: c1ccc(-c2c(-c3ccccc3)c3cc(N(c4cccc(-c5cccc6ccccc56)c4)c4cccc(-c5cccc6ccccc56)c4)ccc3c3ccccc23)cc1 ; c1ccc(-c2c(-c3ccccc3)c3cc(N(c4cccc(-c5cccc6ccccc56)c4)c4ccc5c(ccc6ccccc65)c4)ccc3c3ccccc23)cc1 ; c1ccc(-c2c(-c3ccccc3)c3cc(N(c4cccc(-c5cccc6ccccc56)c4)c4cccc5ccccc45)ccc3c3ccccc23)cc1]

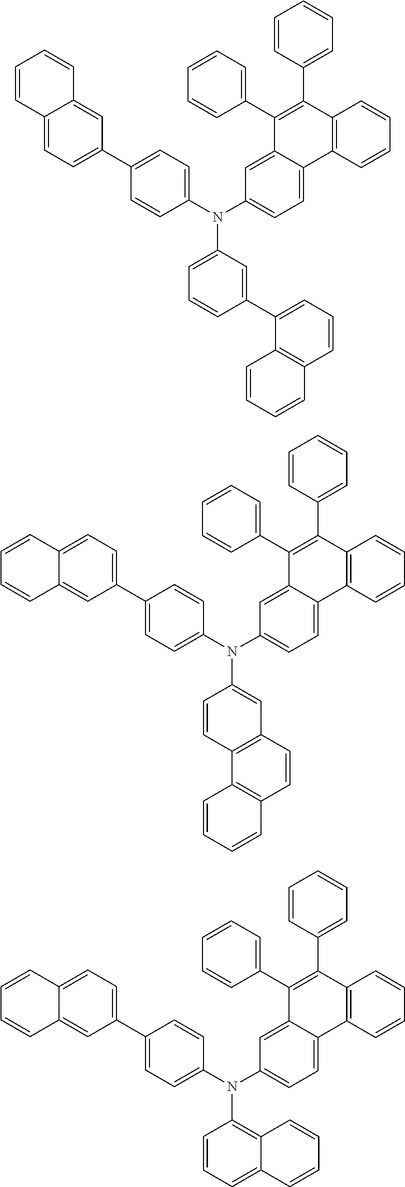

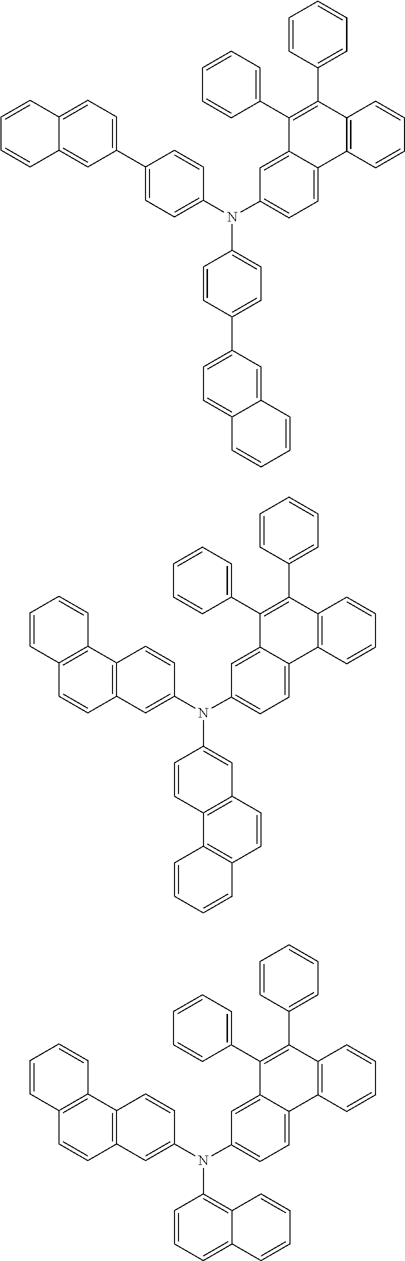

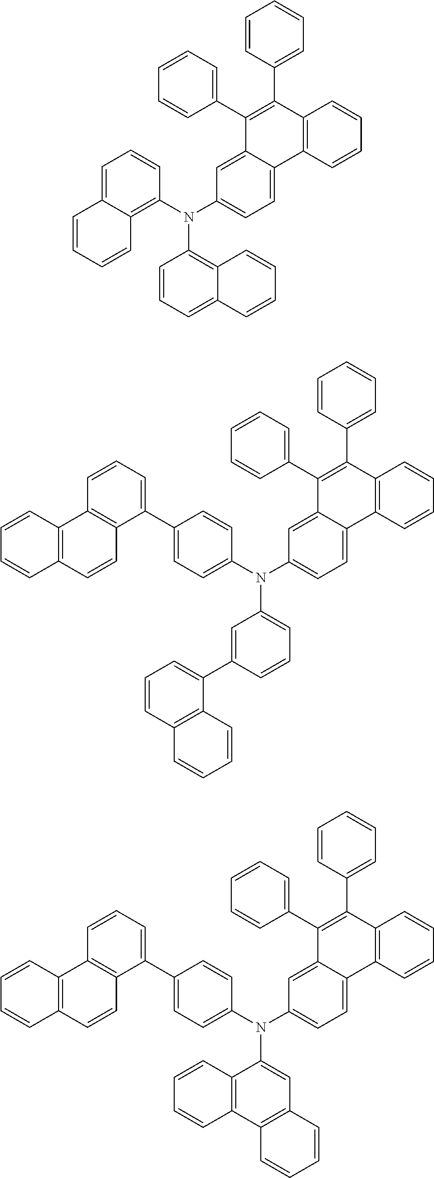 [SMILES: c1ccc(-c2c(-c3ccccc3)c3cc(N(c4cccc5ccccc45)c4cccc5ccccc45)ccc3c3ccccc23)cc1 ; c1ccc(-c2c(-c3ccccc3)c3cc(N(c4ccc(-c5cccc6c5ccc5ccccc56)cc4)c4cccc(-c5cccc6ccccc56)c4)ccc3c3ccccc23)cc1 ; c1ccc(-c2c(-c3ccccc3)c3cc(N(c4ccc(-c5cccc6c5ccc5ccccc56)cc4)c4cc5ccccc5c5ccccc45)ccc3c3ccccc23)cc1]

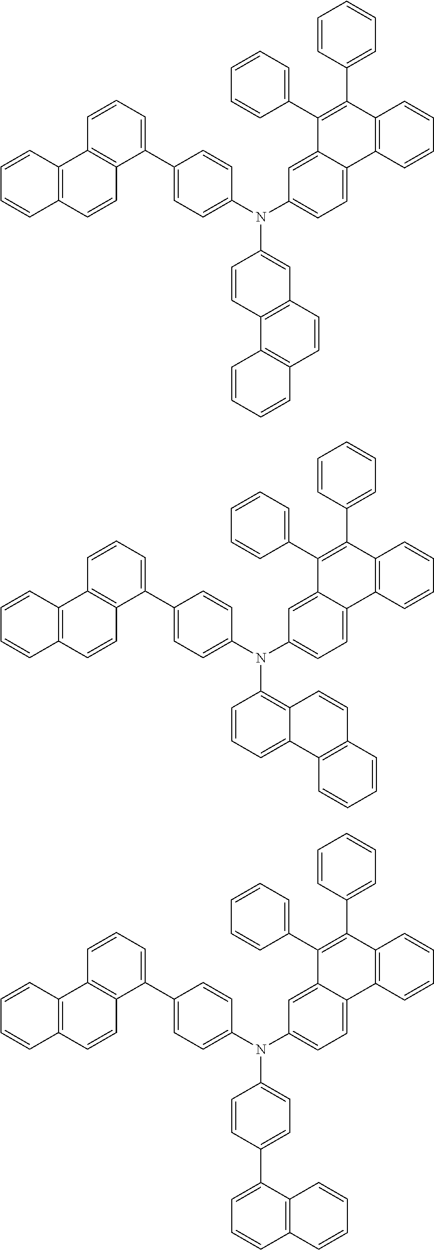

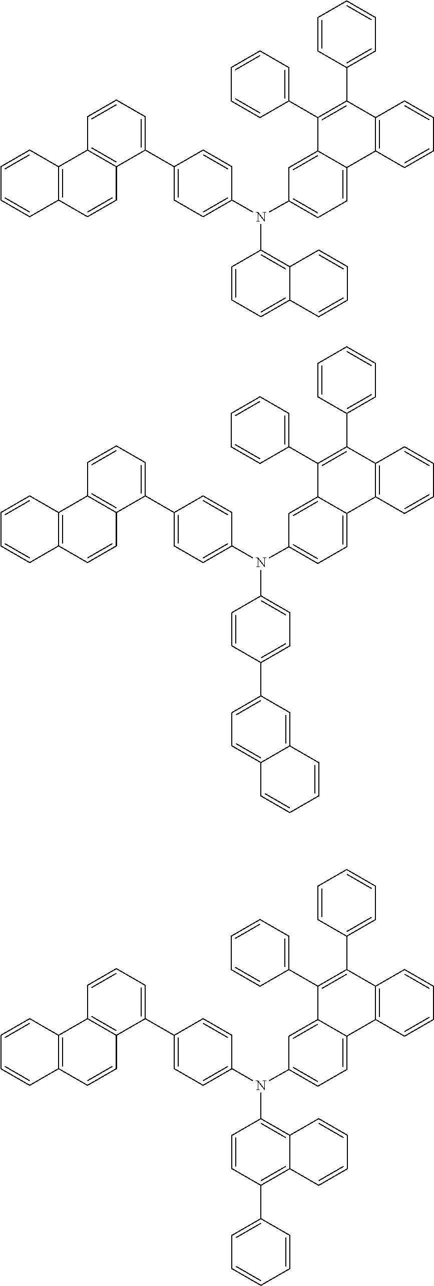

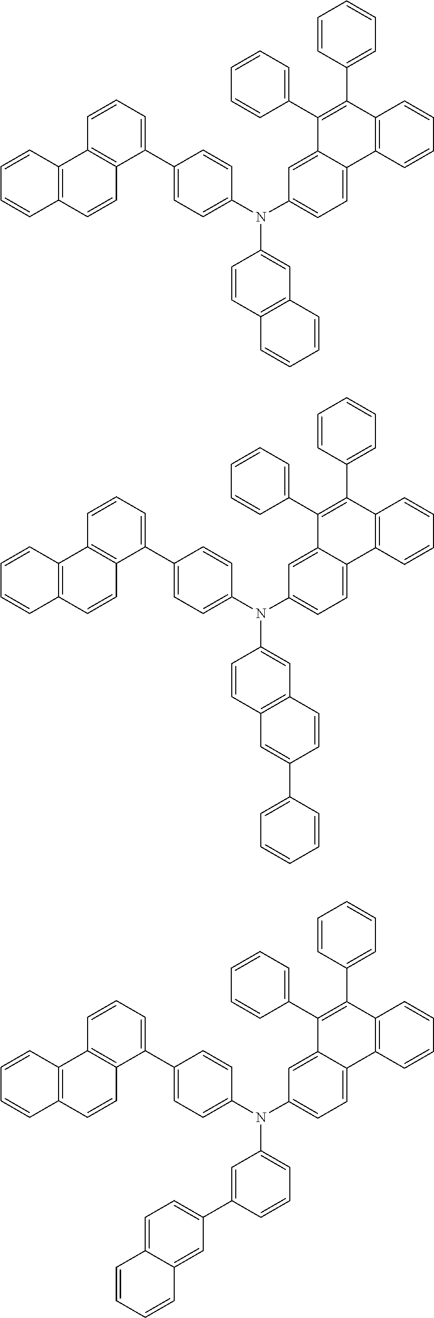 [SMILES: c1ccc(-c2c(-c3ccccc3)c3cc(N(c4ccc(-c5cccc6c5ccc5ccccc56)cc4)c4ccc5ccccc5c4)ccc3c3ccccc23)cc1 ; c1ccc(-c2ccc3cc(N(c4ccc(-c5cccc6c5ccc5ccccc56)cc4)c4ccc5c(c4)c(-c4ccccc4)c(-c4ccccc4)c4ccccc45)ccc3c2)cc1 ; c1ccc(-c2c(-c3ccccc3)c3cc(N(c4ccc(-c5cccc6c5ccc5ccccc56)cc4)c4cccc(-c5ccc6ccccc6c5)c4)ccc3c3ccccc23)cc1]

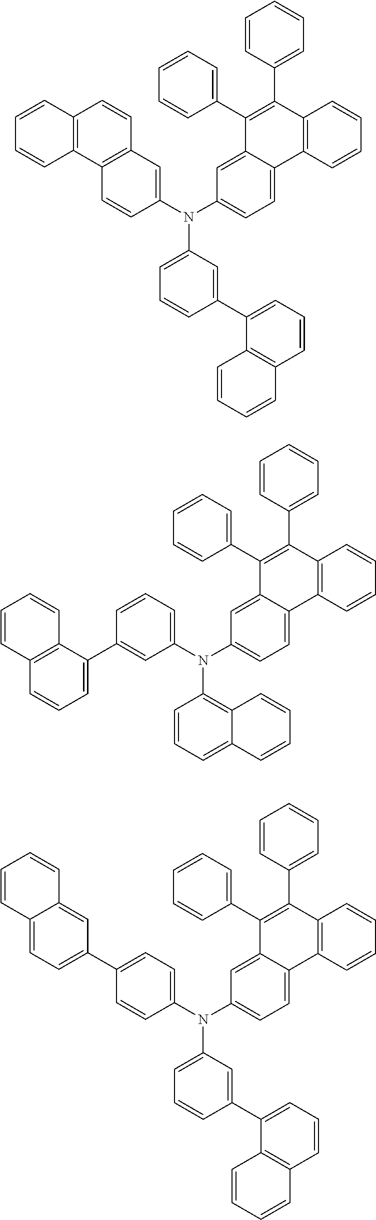

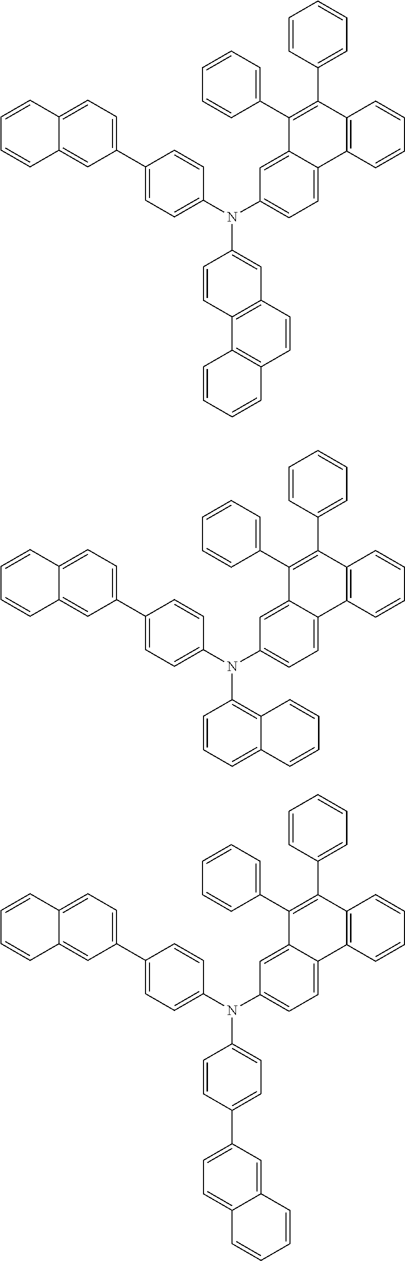

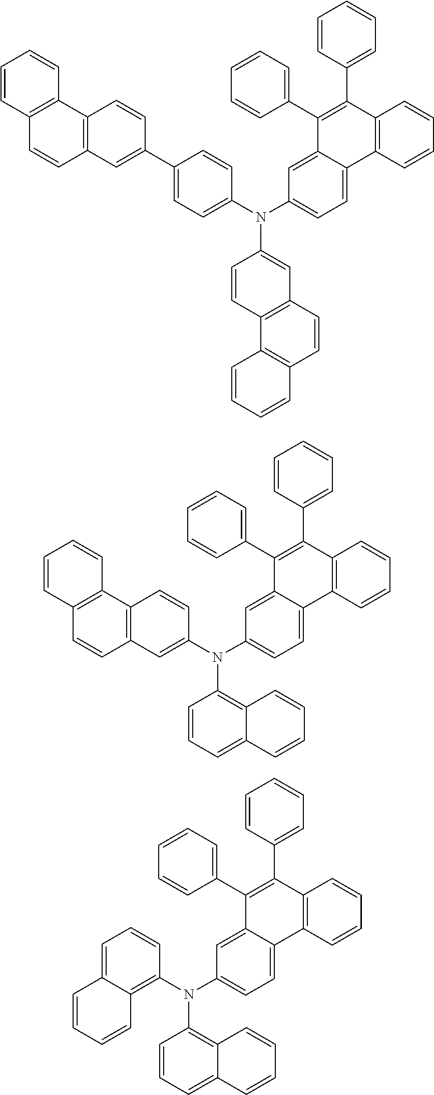

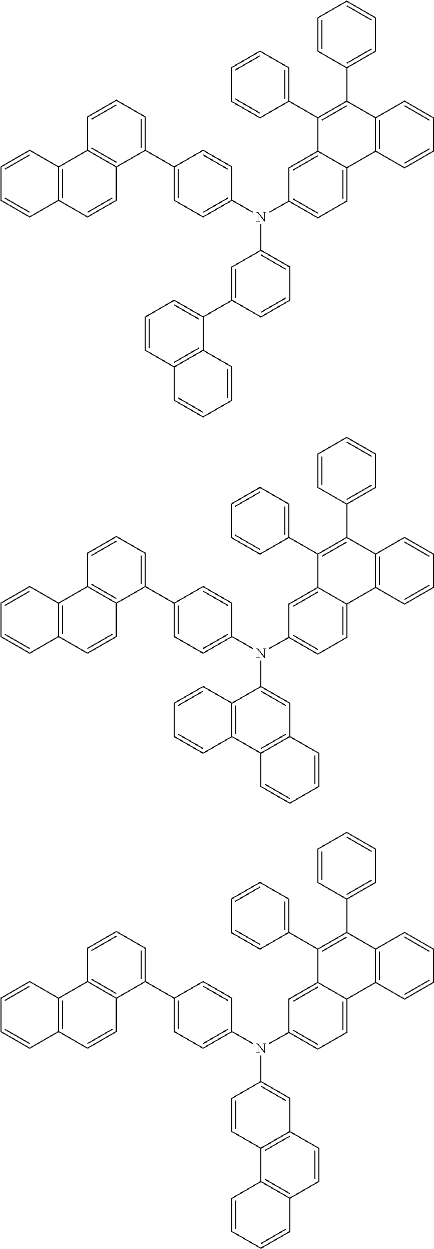 [SMILES: c1ccc(-c2c(-c3ccccc3)c3cc(N(c4ccc(-c5cccc6c5ccc5ccccc56)cc4)c4cccc(-c5cccc6ccccc56)c4)ccc3c3ccccc23)cc1 ; c1ccc(-c2c(-c3ccccc3)c3cc(N(c4ccc(-c5cccc6c5ccc5ccccc56)cc4)c4cc5ccccc5c5ccccc45)ccc3c3ccccc23)cc1 ; c1ccc(-c2c(-c3ccccc3)c3cc(N(c4ccc(-c5cccc6c5ccc5ccccc56)cc4)c4ccc5c(ccc6ccccc65)c4)ccc3c3ccccc23)cc1]

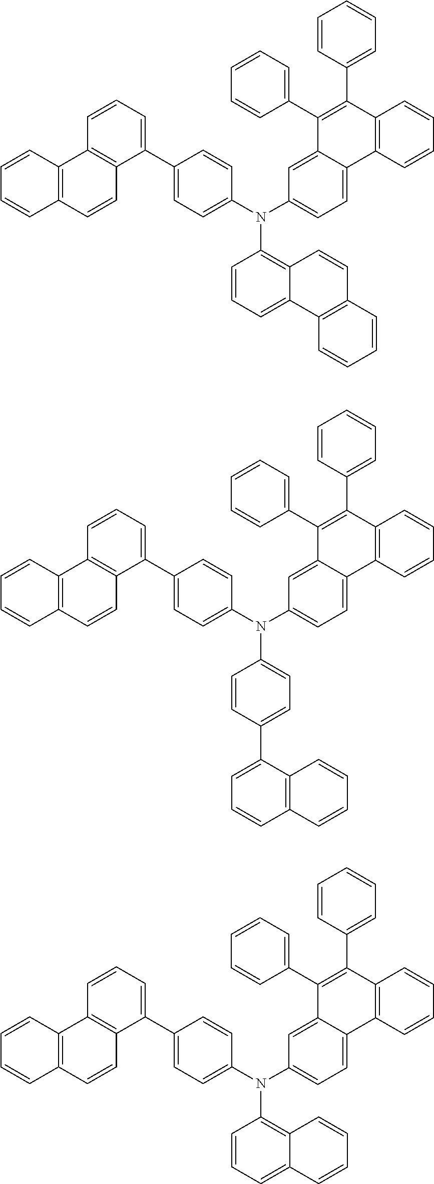

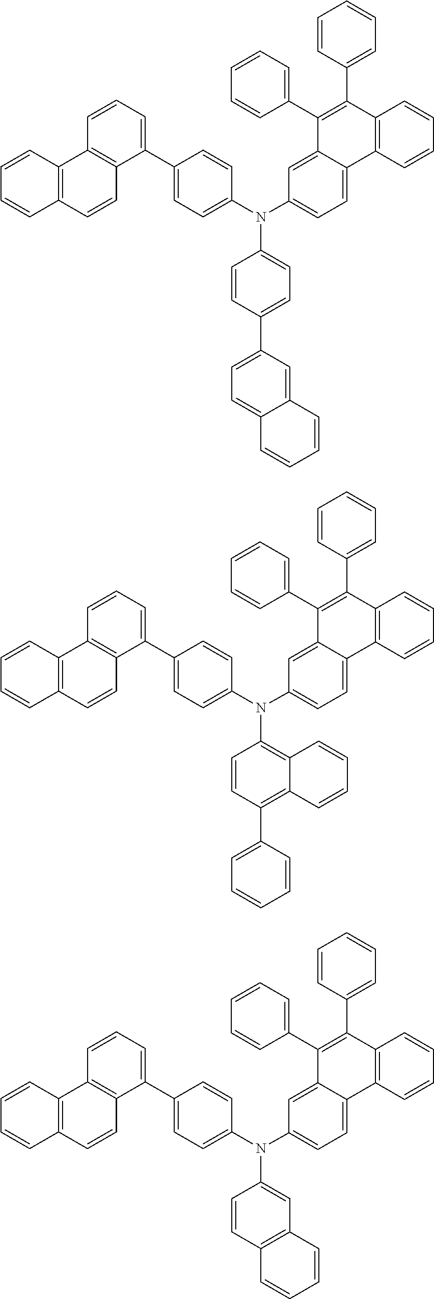 [SMILES: c1ccc(-c2c(-c3ccccc3)c3cc(N(c4ccc(-c5ccc6ccccc6c5)cc4)c4ccc(-c5cccc6c5ccc5ccccc56)cc4)ccc3c3ccccc23)cc1 ; c1ccc(-c2ccc(N(c3ccc(-c4cccc5c4ccc4ccccc45)cc3)c3ccc4c(c3)c(-c3ccccc3)c(-c3ccccc3)c3ccccc34)c3ccccc23)cc1 ; c1ccc(-c2c(-c3ccccc3)c3cc(N(c4ccc(-c5cccc6c5ccc5ccccc56)cc4)c4ccc5ccccc5c4)ccc3c3ccccc23)cc1]

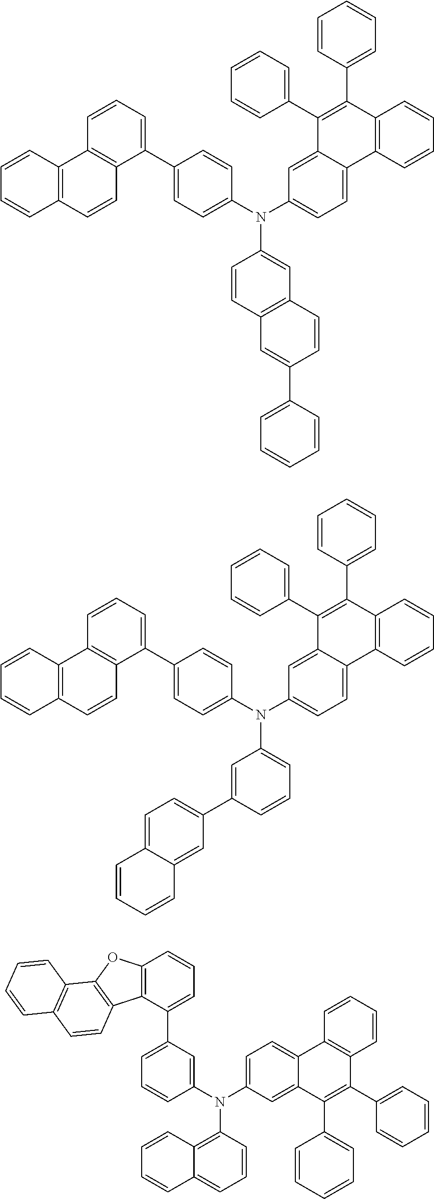

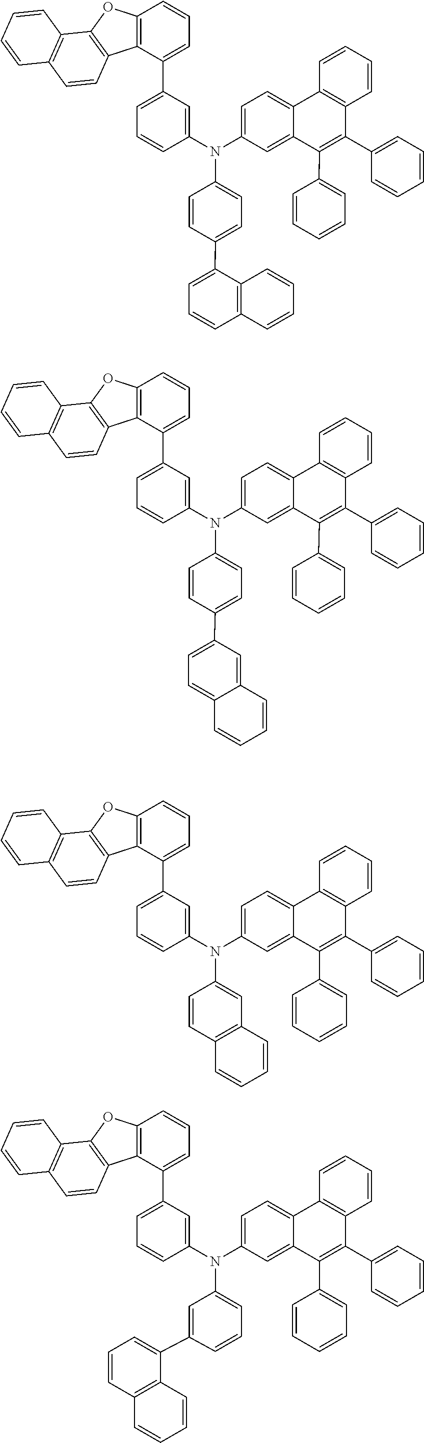 [SMILES: c1ccc(-c2c(-c3ccccc3)c3cc(N(c4ccc(-c5cccc6ccccc56)cc4)c4cccc(-c5cccc6oc7c8ccccc8ccc7c56)c4)ccc3c3ccccc23)cc1 ; c1ccc(-c2c(-c3ccccc3)c3cc(N(c4ccc(-c5ccc6ccccc6c5)cc4)c4cccc(-c5cccc6oc7c8ccccc8ccc7c56)c4)ccc3c3ccccc23)cc1 ; c1ccc(-c2c(-c3ccccc3)c3cc(N(c4cccc(-c5cccc6oc7c8ccccc8ccc7c56)c4)c4ccc5ccccc5c4)ccc3c3ccccc23)cc1 ; c1ccc(-c2c(-c3ccccc3)c3cc(N(c4cccc(-c5cccc6ccccc56)c4)c4cccc(-c5cccc6oc7c8ccccc8ccc7c56)c4)ccc3c3ccccc23)cc1]

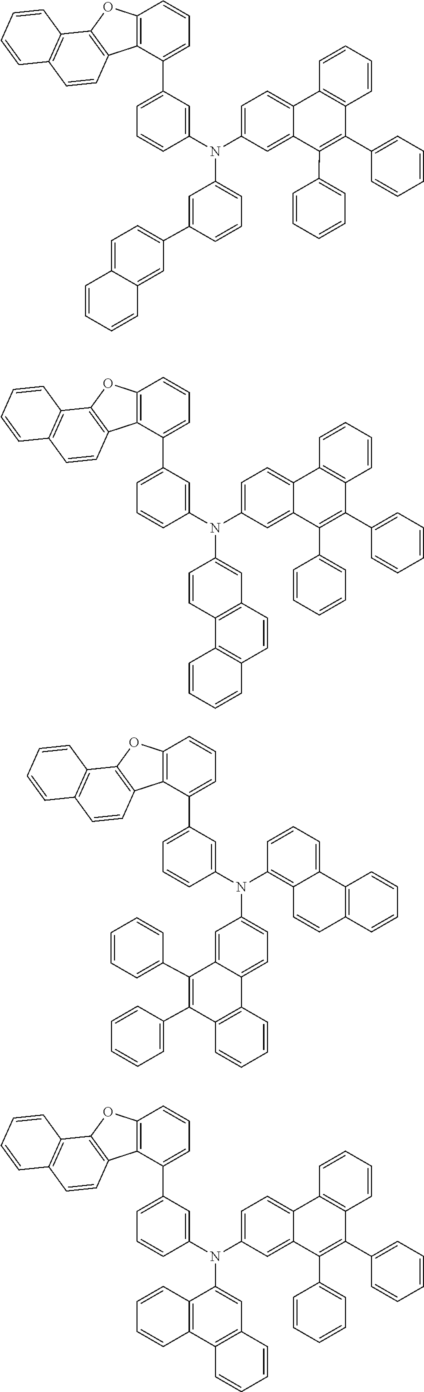

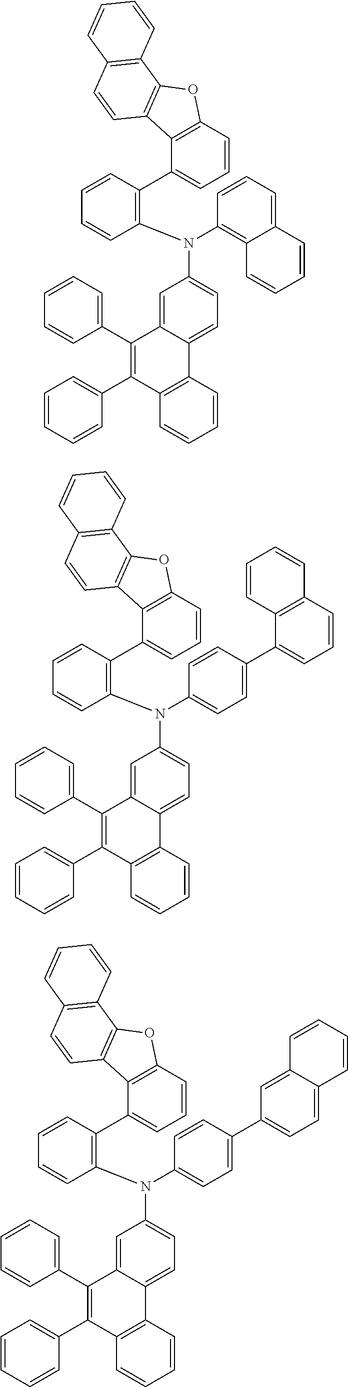

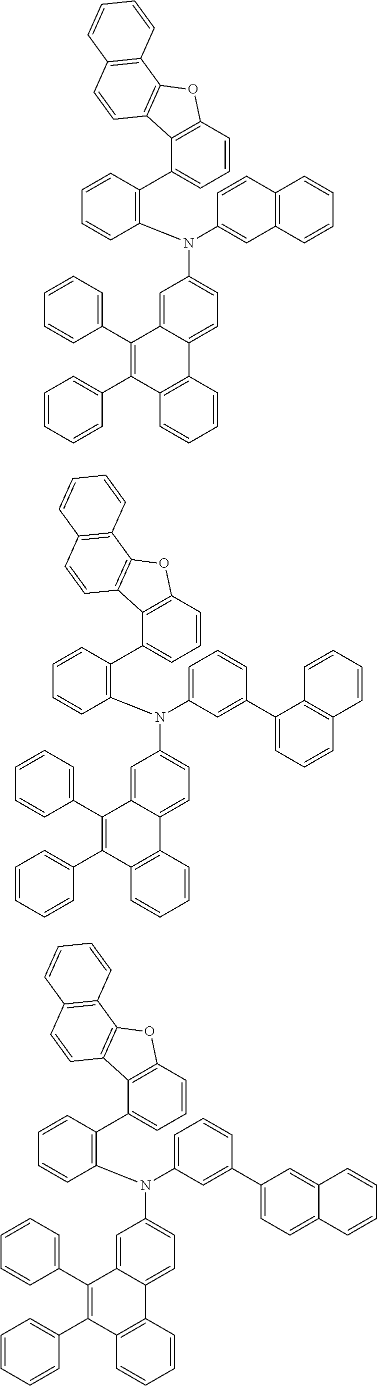

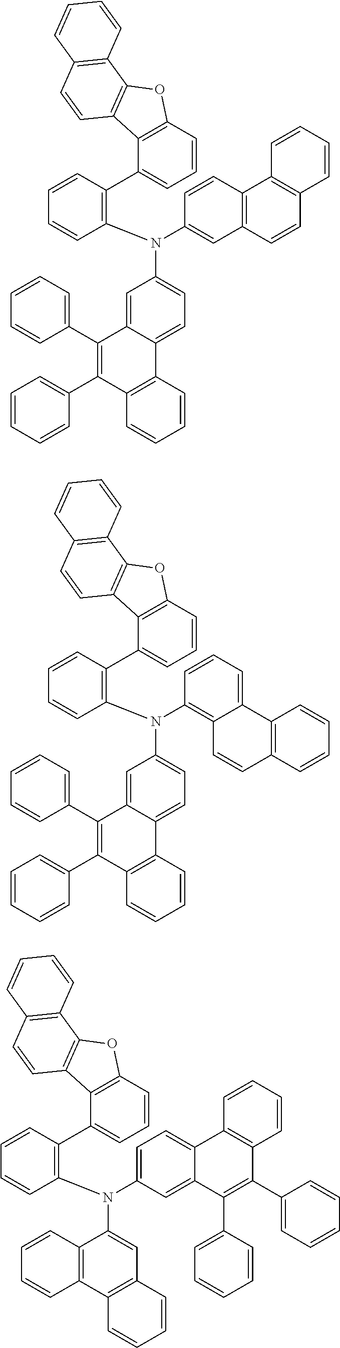

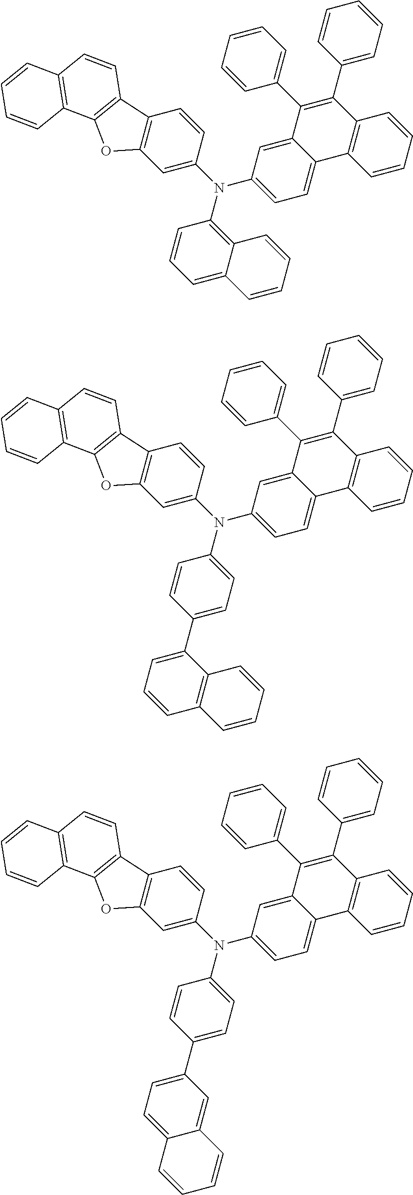 [SMILES: c1ccc(-c2c(-c3ccccc3)c3cc(N(c4ccc5c(c4)oc4c6ccccc6ccc54)c4cccc5ccccc45)ccc3c3ccccc23)cc1 ; c1ccc(-c2c(-c3ccccc3)c3cc(N(c4ccc(-c5cccc6ccccc56)cc4)c4ccc5c(c4)oc4c6ccccc6ccc54)ccc3c3ccccc23)cc1 ; c1ccc(-c2c(-c3ccccc3)c3cc(N(c4ccc(-c5ccc6ccccc6c5)cc4)c4ccc5c(c4)oc4c6ccccc6ccc54)ccc3c3ccccc23)cc1]

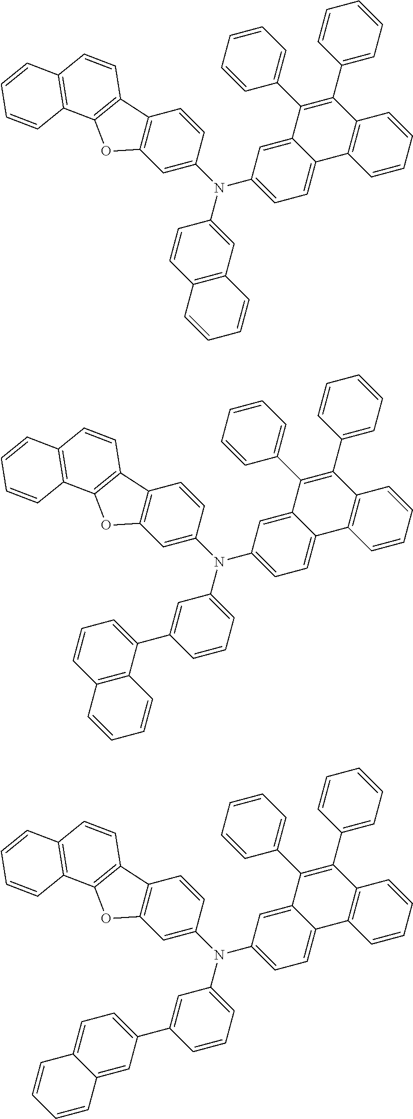 [SMILES: c1ccc(-c2c(-c3ccccc3)c3cc(N(c4ccc5ccccc5c4)c4ccc5c(c4)oc4c6ccccc6ccc54)ccc3c3ccccc23)cc1 ; c1ccc(-c2c(-c3ccccc3)c3cc(N(c4cccc(-c5cccc6ccccc56)c4)c4ccc5c(c4)oc4c6ccccc6ccc54)ccc3c3ccccc23)cc1 ; c1ccc(-c2c(-c3ccccc3)c3cc(N(c4cccc(-c5ccc6ccccc6c5)c4)c4ccc5c(c4)oc4c6ccccc6ccc54)ccc3c3ccccc23)cc1]

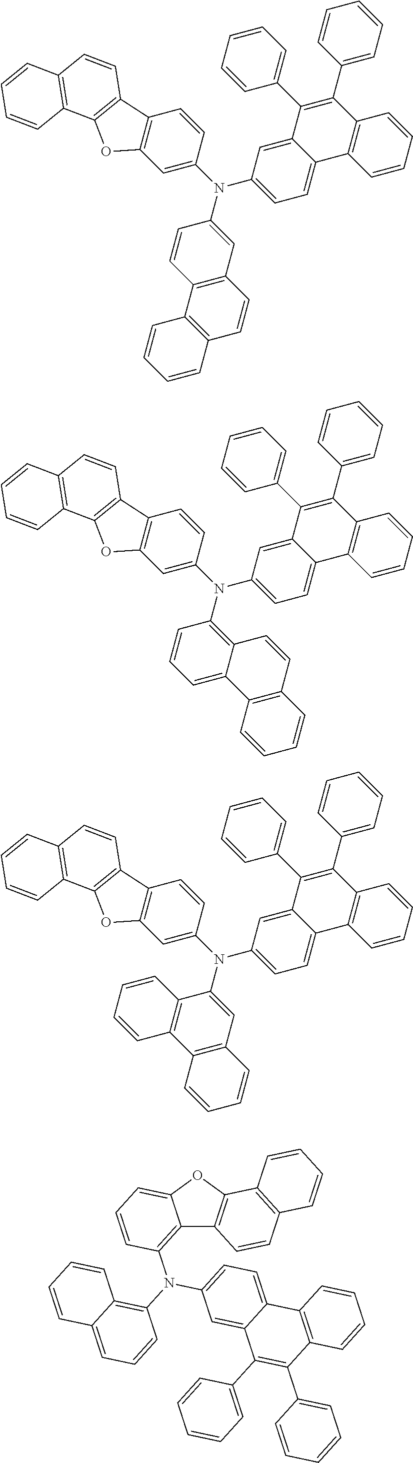

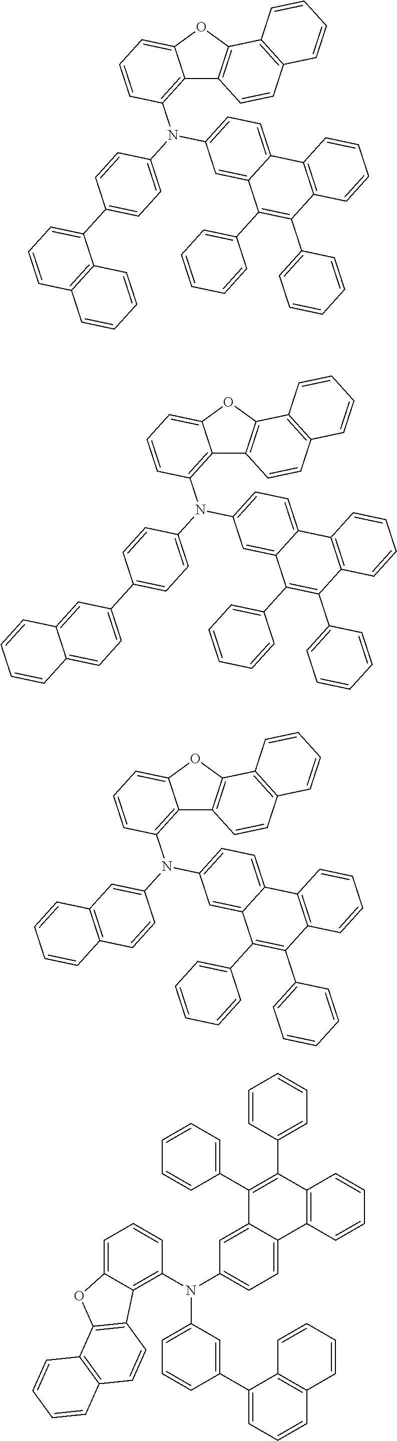

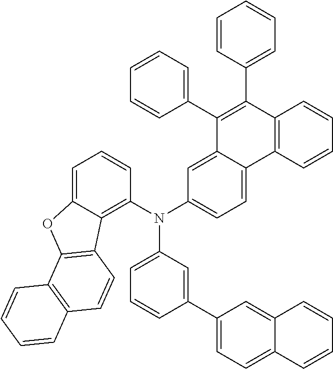 [SMILES: c1ccc(-c2c(-c3ccccc3)c3cc(N(c4cccc(-c5ccc6ccccc6c5)c4)c4cccc5oc6c7ccccc7ccc6c45)ccc3c3ccccc23)cc1]

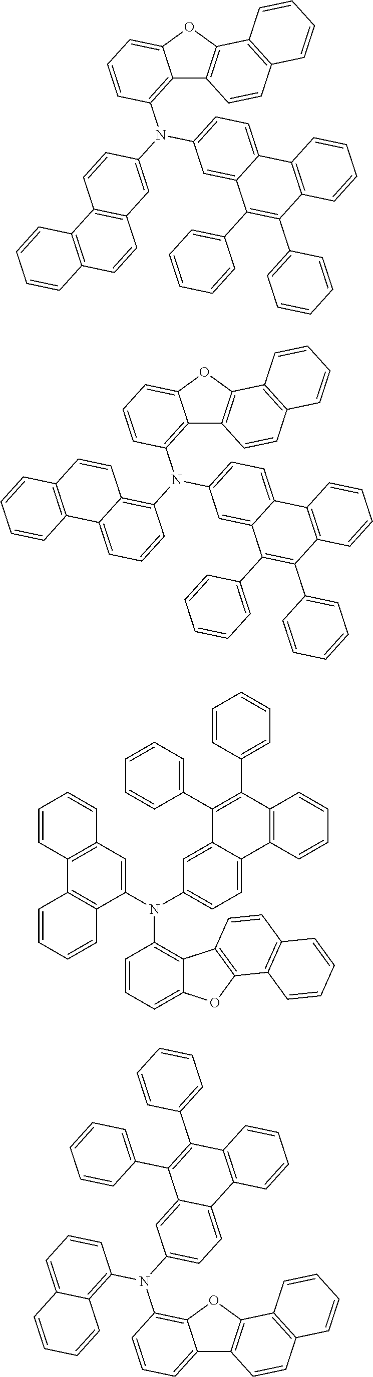

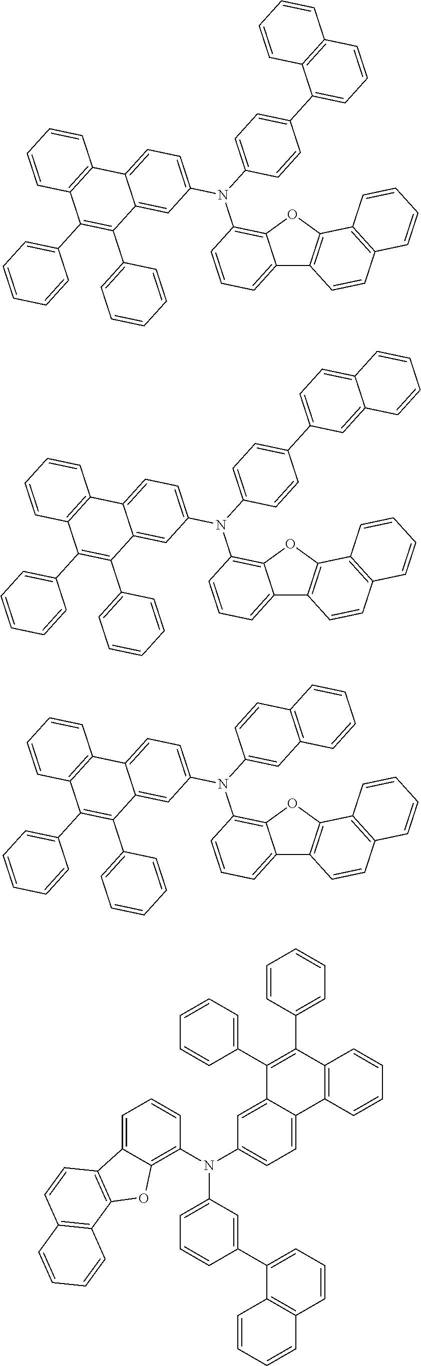 [SMILES: c1ccc(-c2c(-c3ccccc3)c3cc(N(c4ccc(-c5cccc6ccccc56)cc4)c4cccc5c4oc4c6ccccc6ccc54)ccc3c3ccccc23)cc1 ; c1ccc(-c2c(-c3ccccc3)c3cc(N(c4ccc(-c5ccc6ccccc6c5)cc4)c4cccc5c4oc4c6ccccc6ccc54)ccc3c3ccccc23)cc1 ; c1ccc(-c2c(-c3ccccc3)c3cc(N(c4ccc5ccccc5c4)c4cccc5c4oc4c6ccccc6ccc54)ccc3c3ccccc23)cc1 ; c1ccc(-c2c(-c3ccccc3)c3cc(N(c4cccc(-c5cccc6ccccc56)c4)c4cccc5c4oc4c6ccccc6ccc54)ccc3c3ccccc23)cc1]

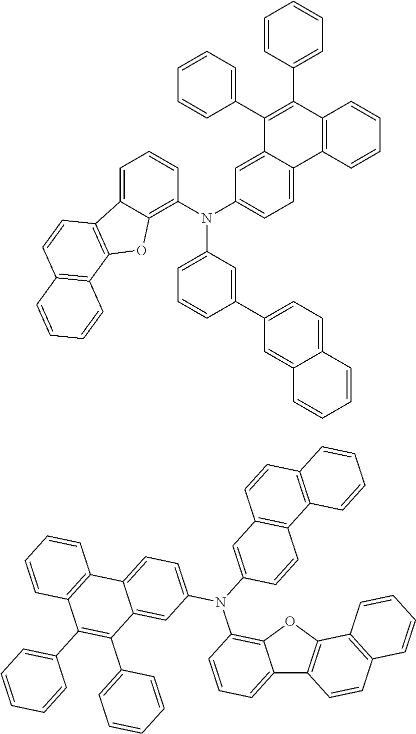

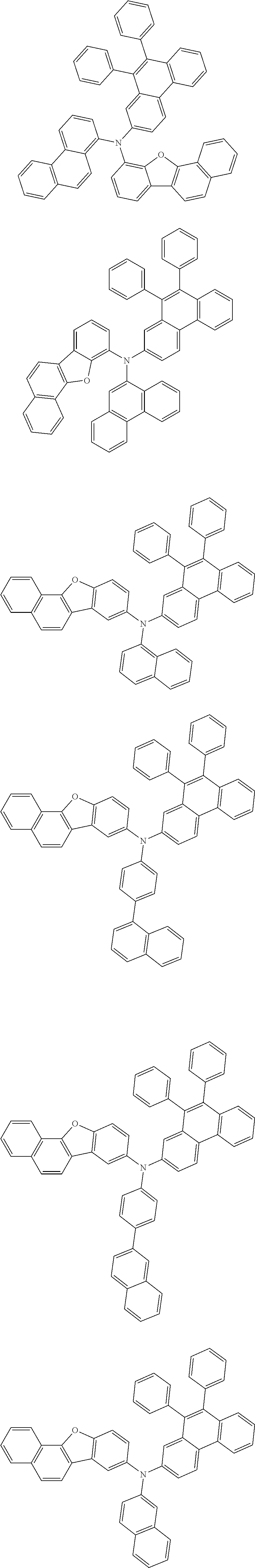

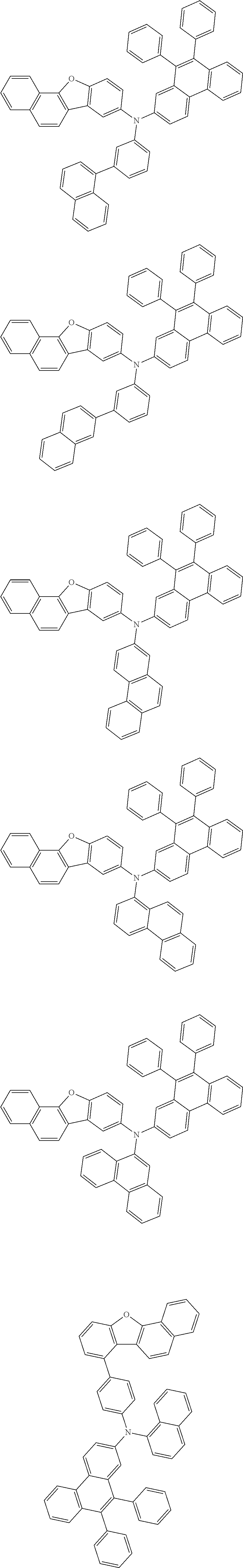

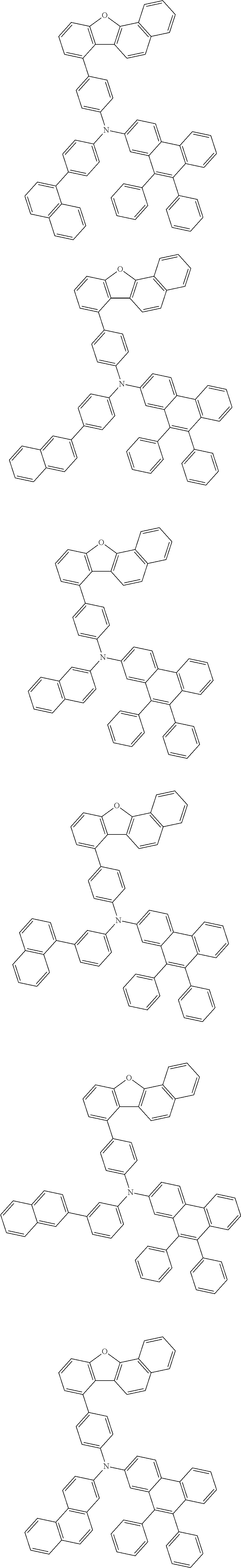

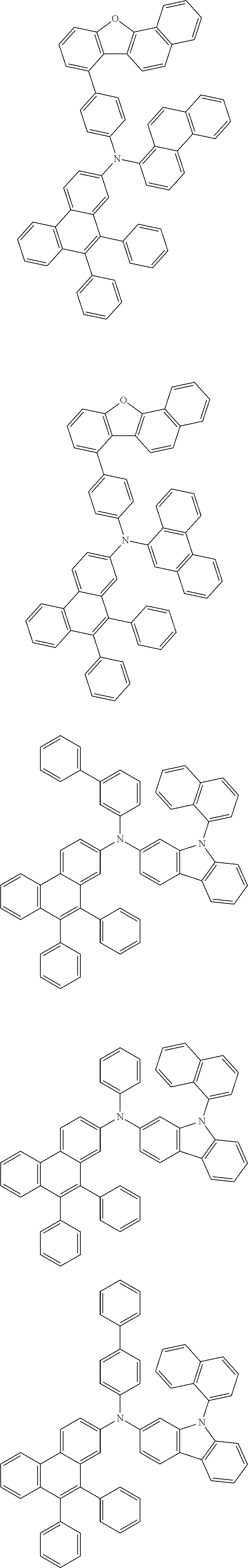

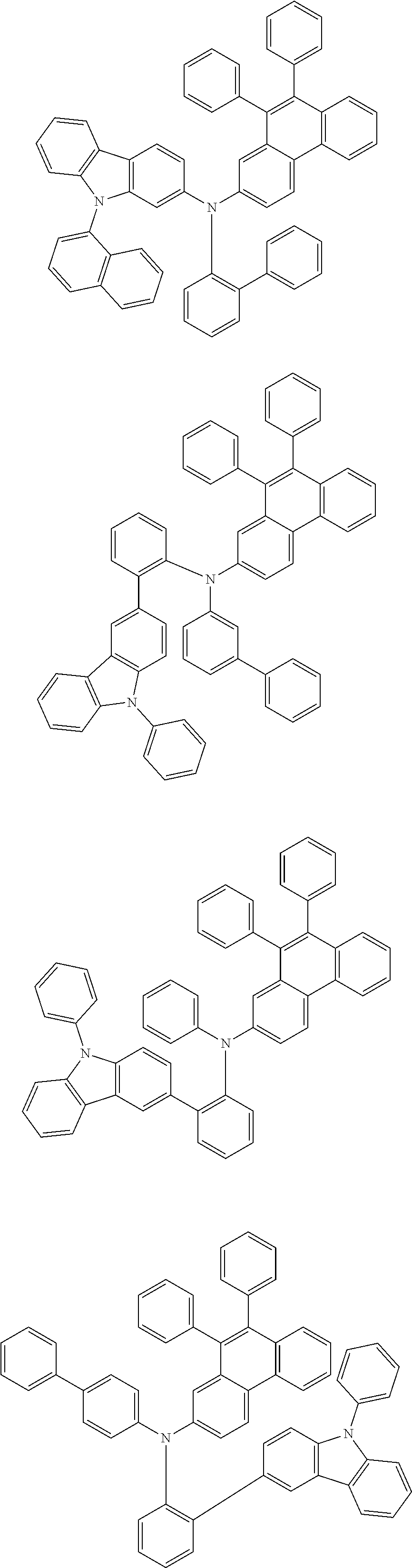 [SMILES: c1ccc(-c2ccccc2N(c2ccc3c(c2)c(-c2ccccc2)c(-c2ccccc2)c2ccccc23)c2ccc3c4ccccc4n(-c4cccc5ccccc45)c3c2)cc1 ; c1ccc(-c2cccc(N(c3ccc4c(c3)c(-c3ccccc3)c(-c3ccccc3)c3ccccc34)c3ccccc3-c3ccc4c(c3)c3ccccc3n4-c3ccccc3)c2)cc1 ; c1ccc(-c2c(-c3ccccc3)c3cc(N(c4ccccc4)c4ccccc4-c4ccc5c(c4)c4ccccc4n5-c4ccccc4)ccc3c3ccccc23)cc1 ; c1ccc(-c2ccc(N(c3ccc4c(c3)c(-c3ccccc3)c(-c3ccccc3)c3ccccc34)c3ccccc3-c3ccc4c(c3)c3ccccc3n4-c3ccccc3)cc2)cc1]

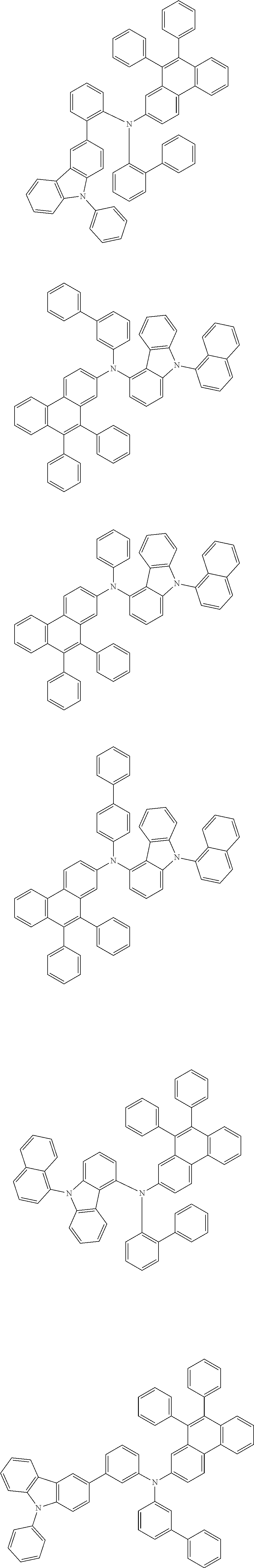

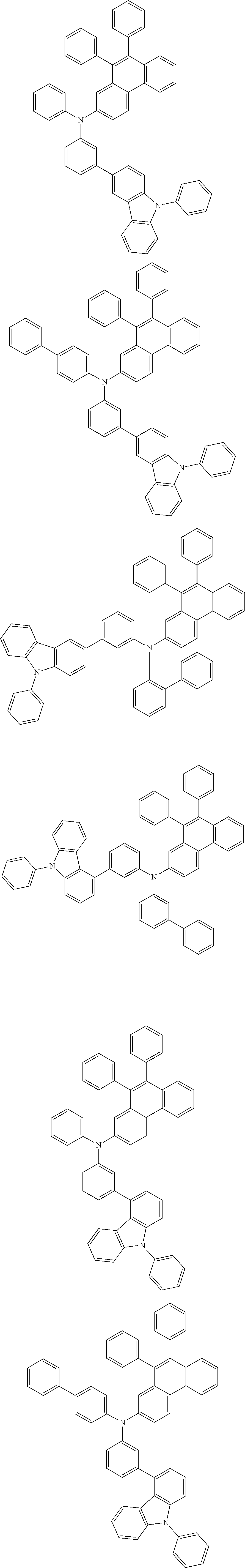

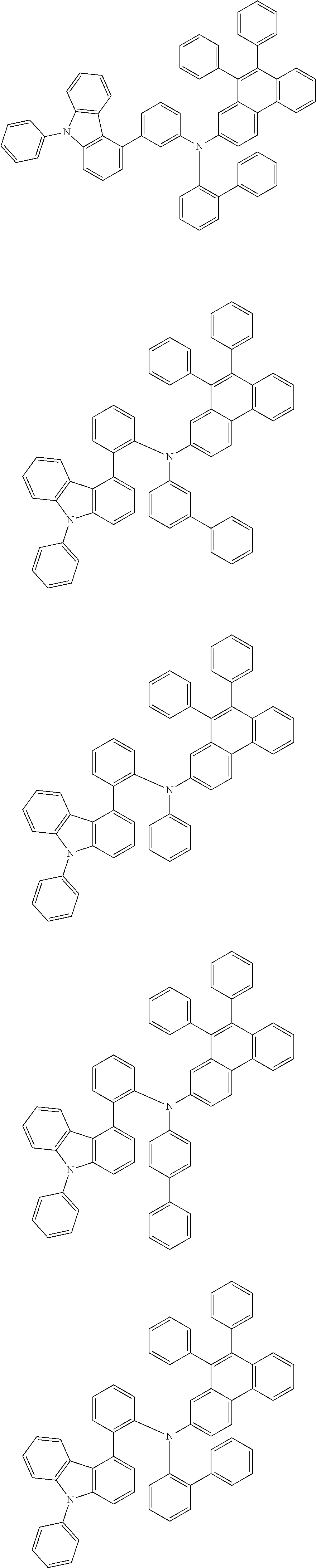

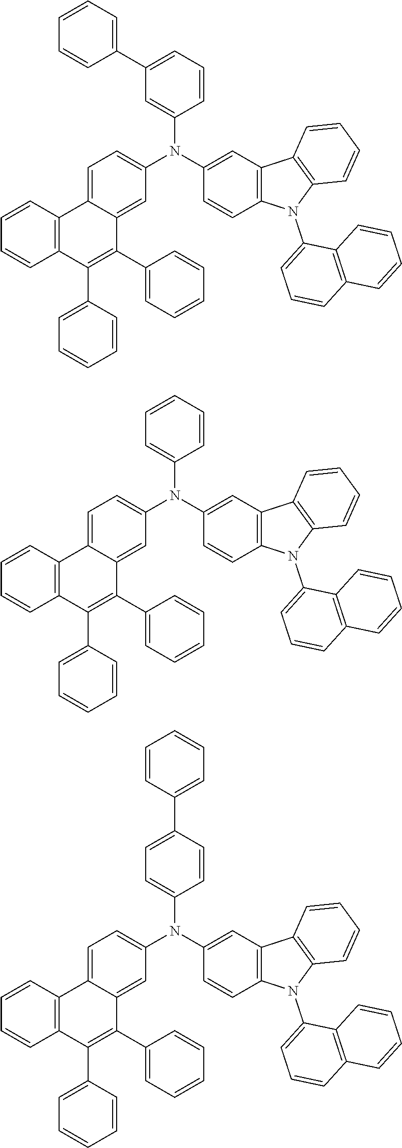 [SMILES: c1ccc(-c2cccc(N(c3ccc4c(c3)c(-c3ccccc3)c(-c3ccccc3)c3ccccc34)c3ccc4c(c3)c3ccccc3n4-c3cccc4ccccc34)c2)cc1 ; c1ccc(-c2c(-c3ccccc3)c3cc(N(c4ccccc4)c4ccc5c(c4)c4ccccc4n5-c4cccc5ccccc45)ccc3c3ccccc23)cc1 ; c1ccc(-c2ccc(N(c3ccc4c(c3)c(-c3ccccc3)c(-c3ccccc3)c3ccccc34)c3ccc4c(c3)c3ccccc3n4-c3cccc4ccccc34)cc2)cc1]

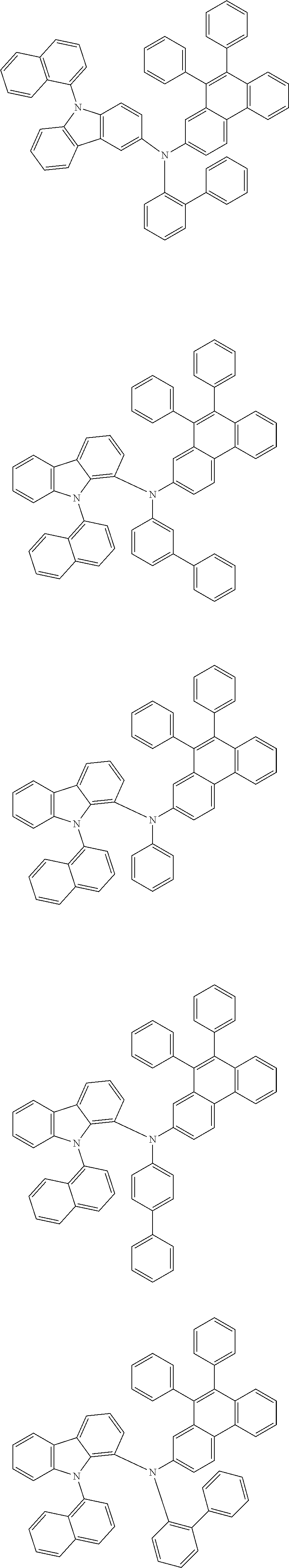

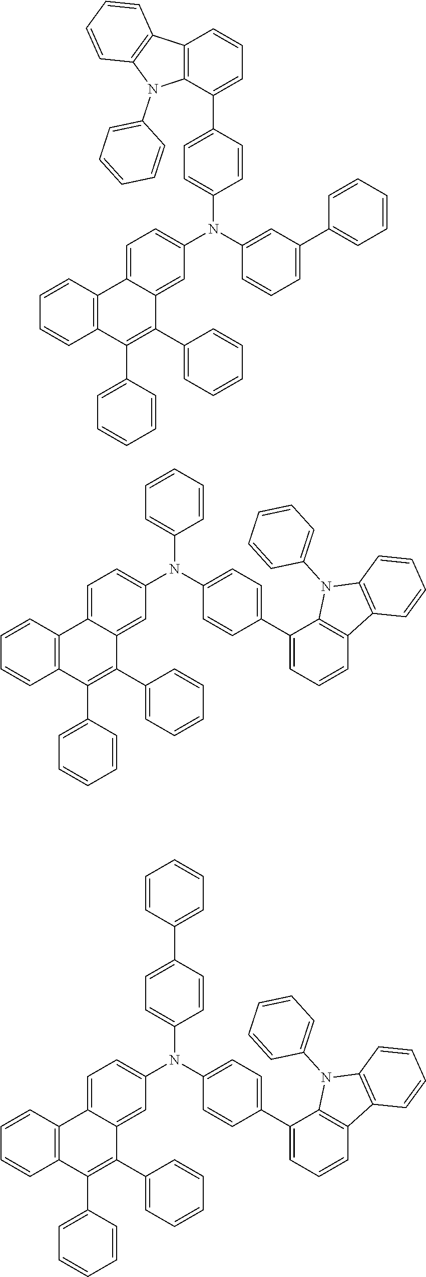

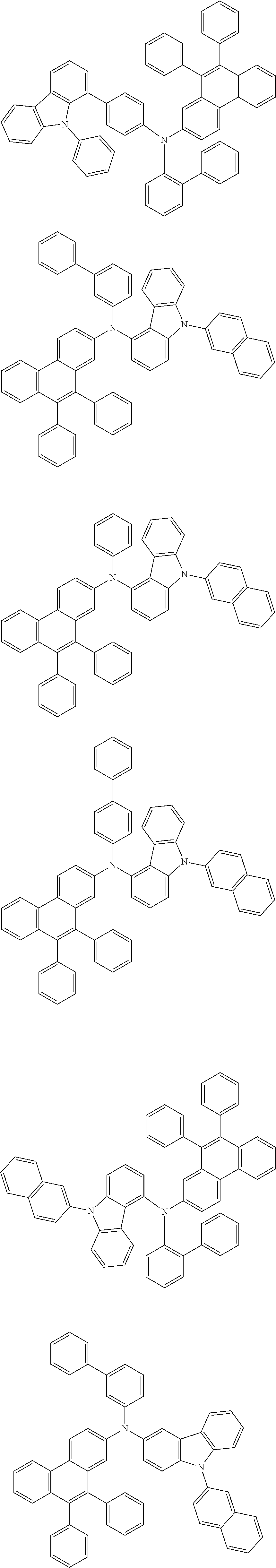

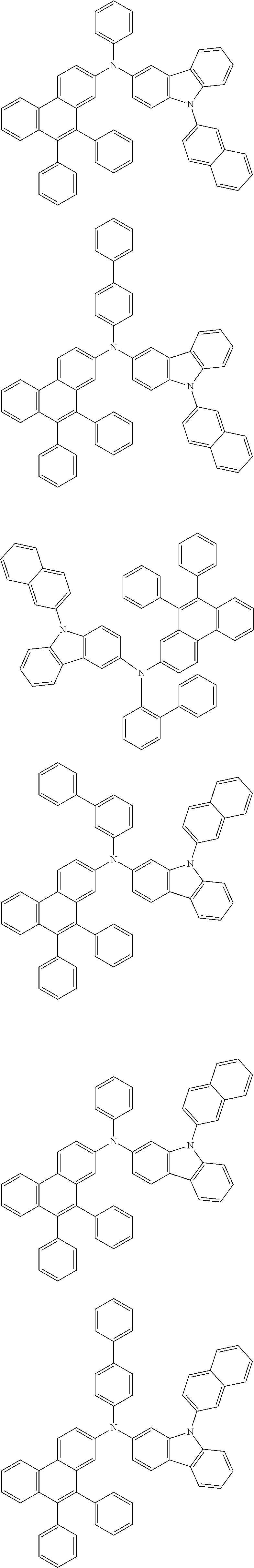

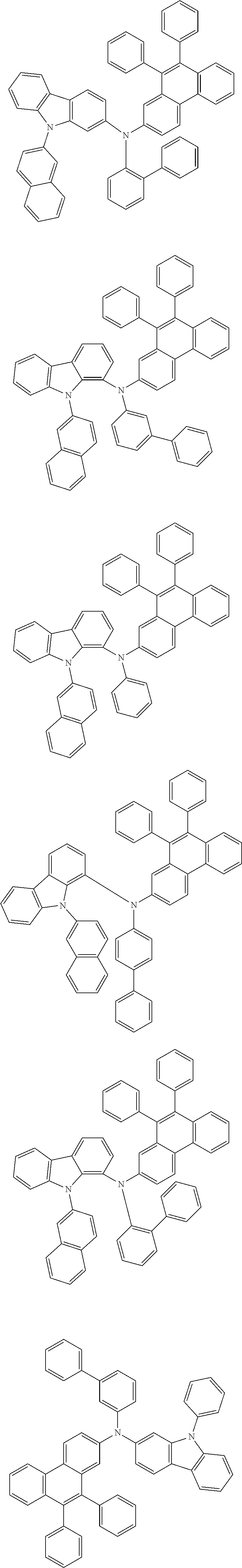

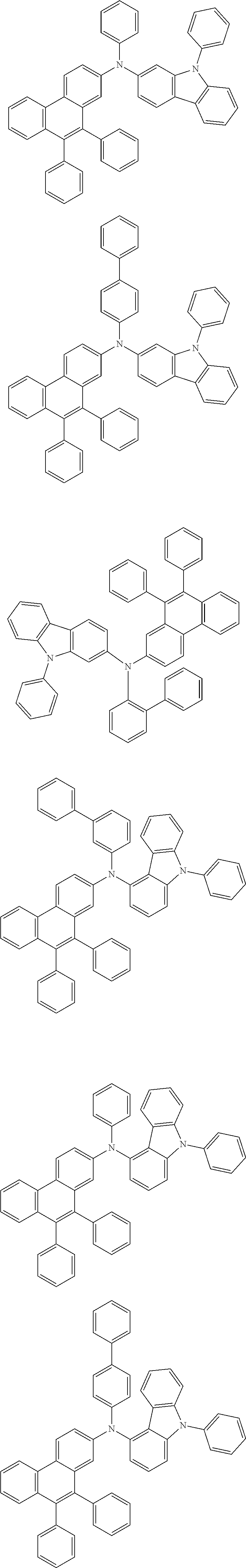

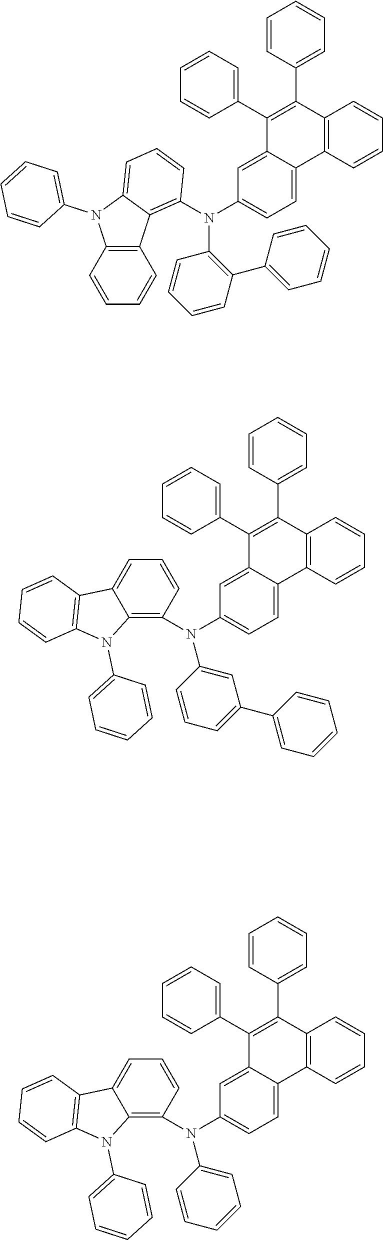 [SMILES: c1ccc(-c2ccccc2N(c2ccc3c(c2)c(-c2ccccc2)c(-c2ccccc2)c2ccccc23)c2cccc3c2c2ccccc2n3-c2ccccc2)cc1 ; c1ccc(-c2cccc(N(c3ccc4c(c3)c(-c3ccccc3)c(-c3ccccc3)c3ccccc34)c3cccc4c5ccccc5n(-c5ccccc5)c34)c2)cc1 ; c1ccc(-c2c(-c3ccccc3)c3cc(N(c4ccccc4)c4cccc5c6ccccc6n(-c6ccccc6)c45)ccc3c3ccccc23)cc1]

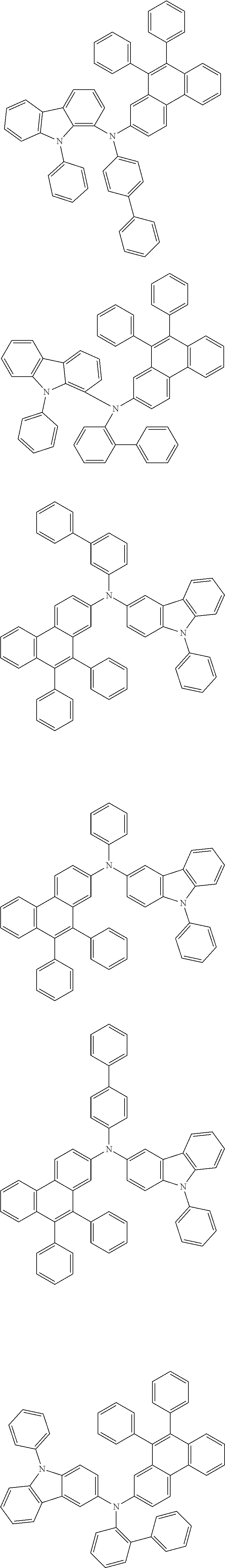

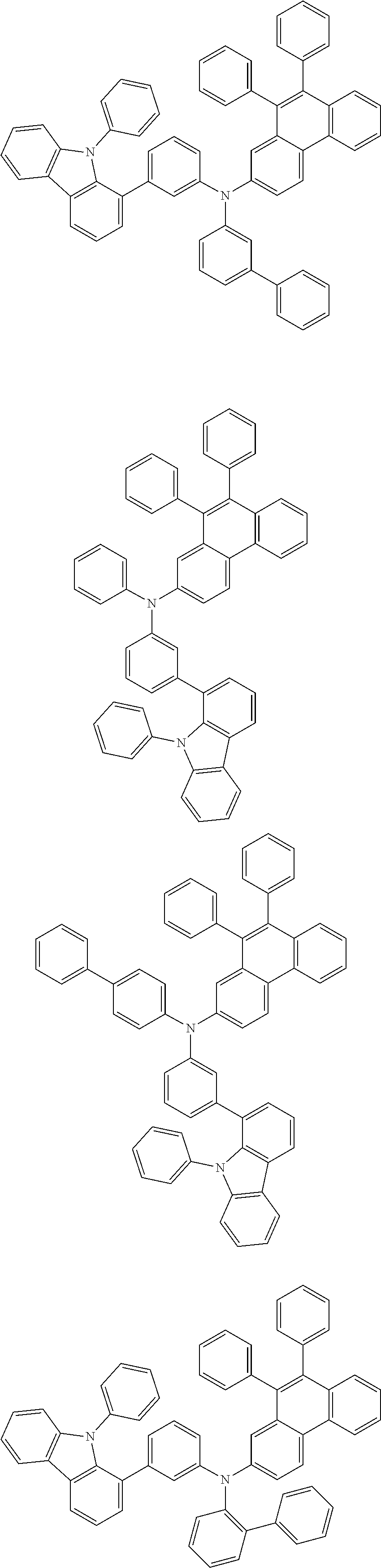

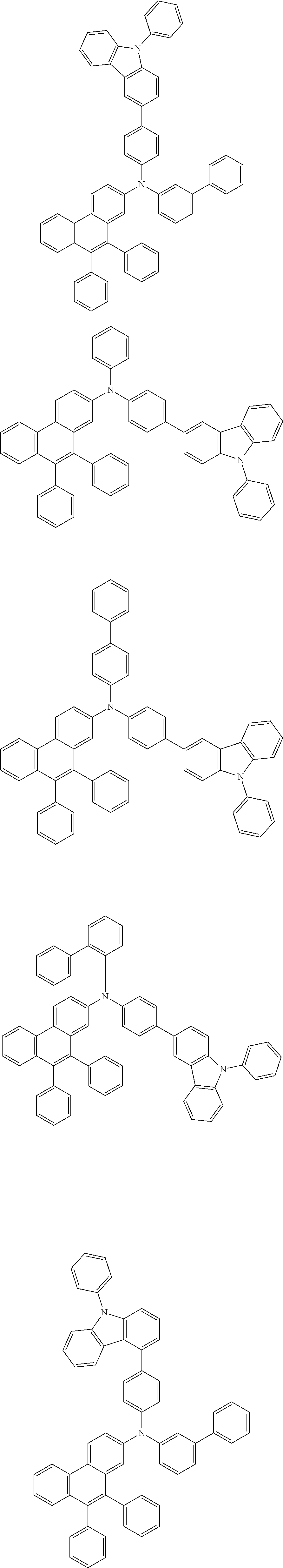

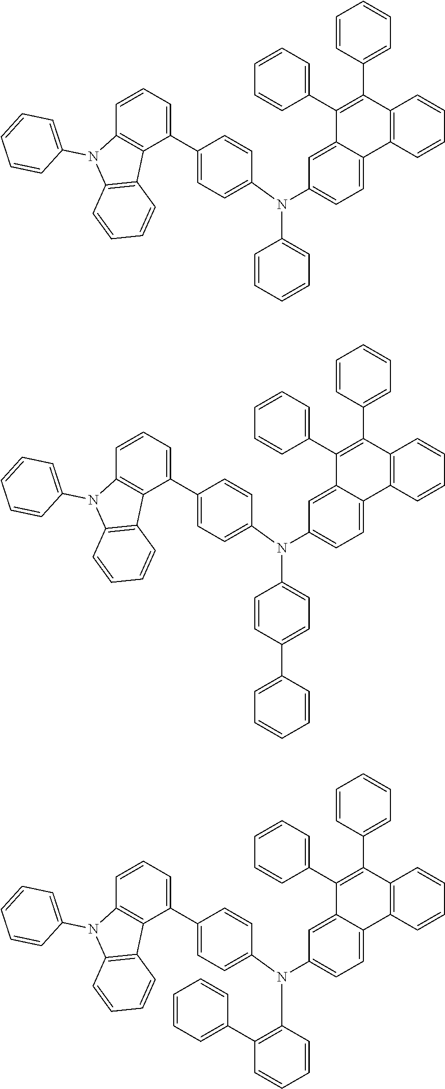 [SMILES: c1ccc(-c2c(-c3ccccc3)c3cc(N(c4ccccc4)c4ccc(-c5cccc6c5c5ccccc5n6-c5ccccc5)cc4)ccc3c3ccccc23)cc1 ; c1ccc(-c2ccc(N(c3ccc(-c4cccc5c4c4ccccc4n5-c4ccccc4)cc3)c3ccc4c(c3)c(-c3ccccc3)c(-c3ccccc3)c3ccccc34)cc2)cc1 ; c1ccc(-c2ccccc2N(c2ccc(-c3cccc4c3c3ccccc3n4-c3ccccc3)cc2)c2ccc3c(c2)c(-c2ccccc2)c(-c2ccccc2)c2ccccc23)cc1]

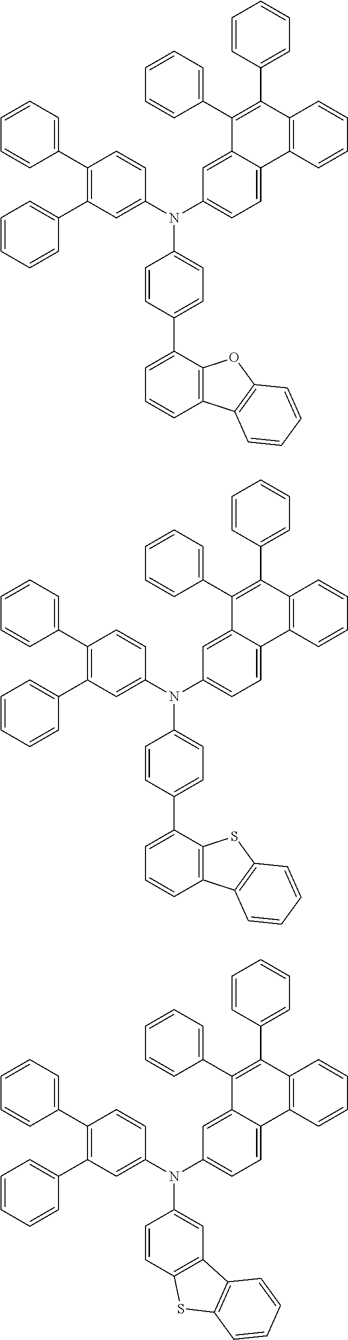

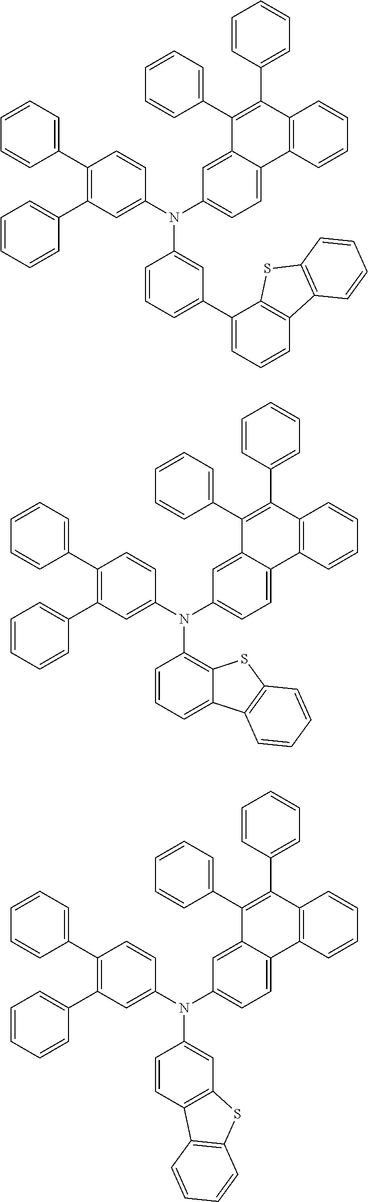

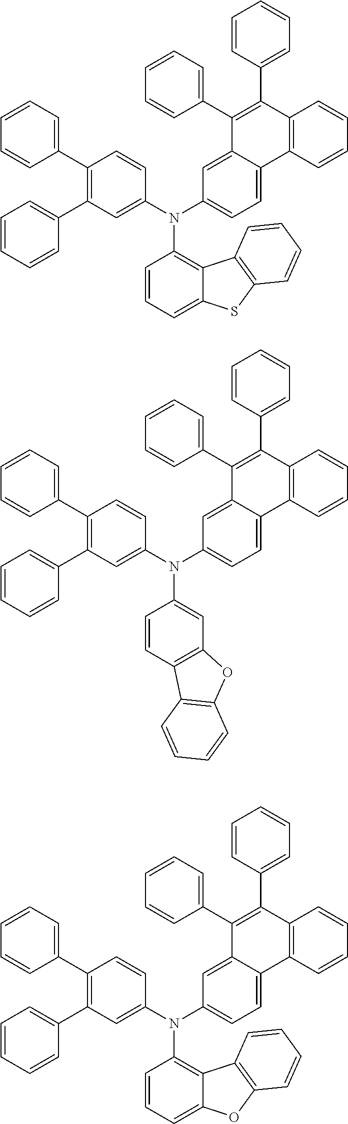

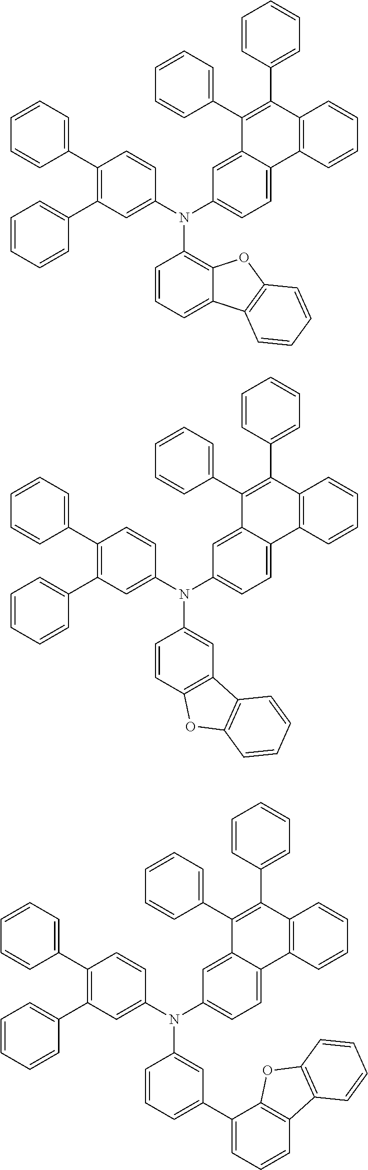

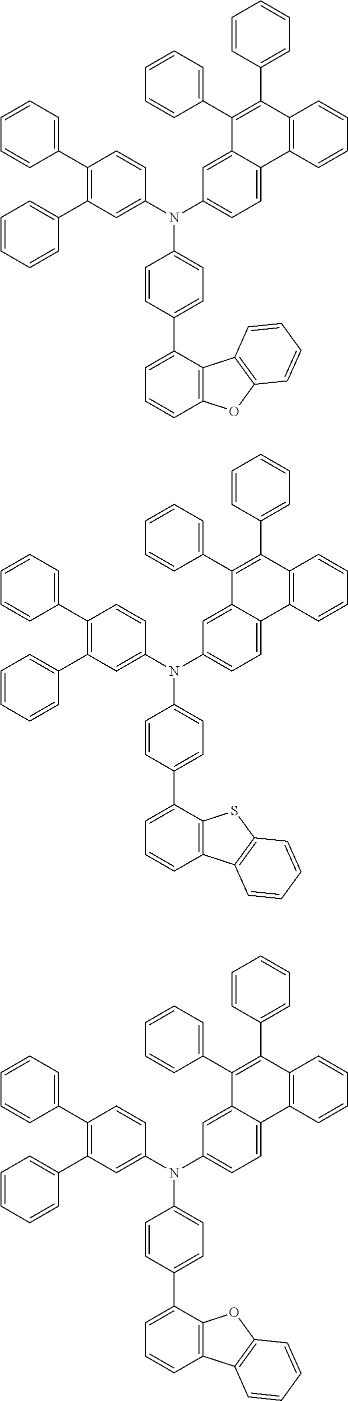 [SMILES: c1ccc(-c2ccc(N(c3ccc(-c4cccc5oc6ccccc6c45)cc3)c3ccc4c(c3)c(-c3ccccc3)c(-c3ccccc3)c3ccccc34)cc2-c2ccccc2)cc1 ; c1ccc(-c2ccc(N(c3ccc(-c4cccc5c4sc4ccccc45)cc3)c3ccc4c(c3)c(-c3ccccc3)c(-c3ccccc3)c3ccccc34)cc2-c2ccccc2)cc1 ; c1ccc(-c2ccc(N(c3ccc(-c4cccc5c4oc4ccccc45)cc3)c3ccc4c(c3)c(-c3ccccc3)c(-c3ccccc3)c3ccccc34)cc2-c2ccccc2)cc1]

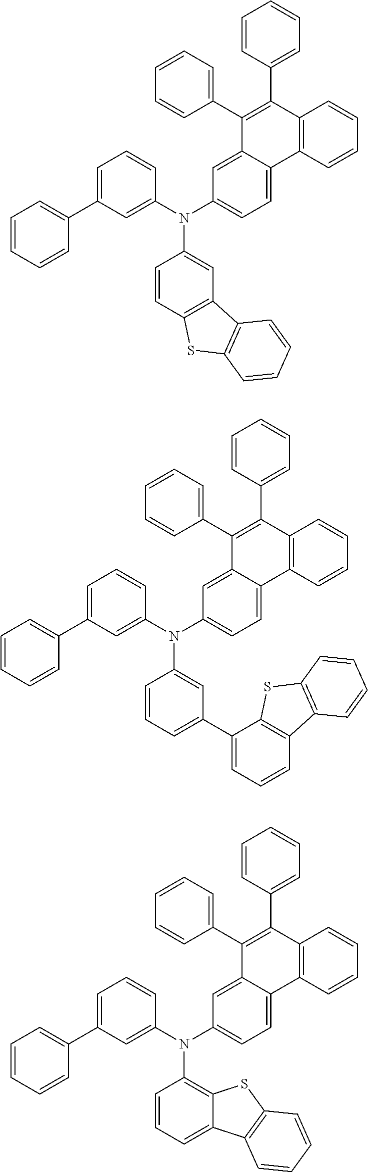

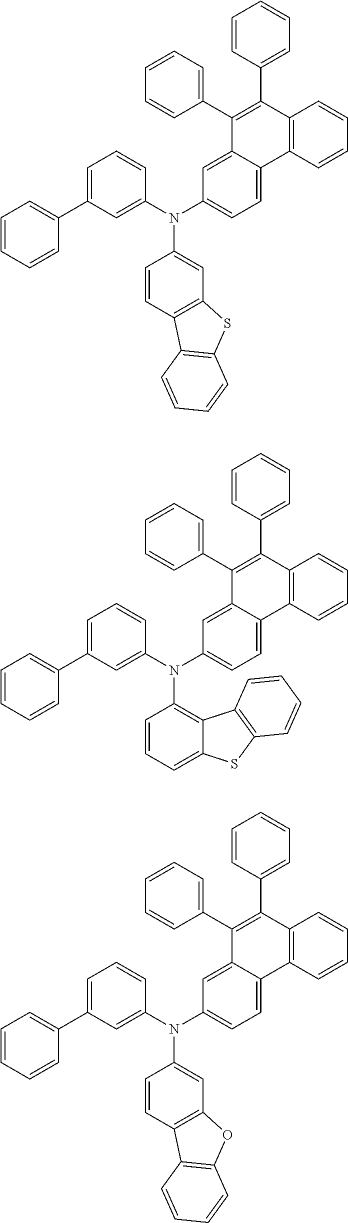 [SMILES: c1ccc(-c2cccc(N(c3ccc4c(c3)sc3ccccc34)c3ccc4c(c3)c(-c3ccccc3)c(-c3ccccc3)c3ccccc34)c2)cc1 ; c1ccc(-c2cccc(N(c3ccc4c(c3)c(-c3ccccc3)c(-c3ccccc3)c3ccccc34)c3cccc4sc5ccccc5c34)c2)cc1 ; c1ccc(-c2cccc(N(c3ccc4c(c3)oc3ccccc34)c3ccc4c(c3)c(-c3ccccc3)c(-c3ccccc3)c3ccccc34)c2)cc1]

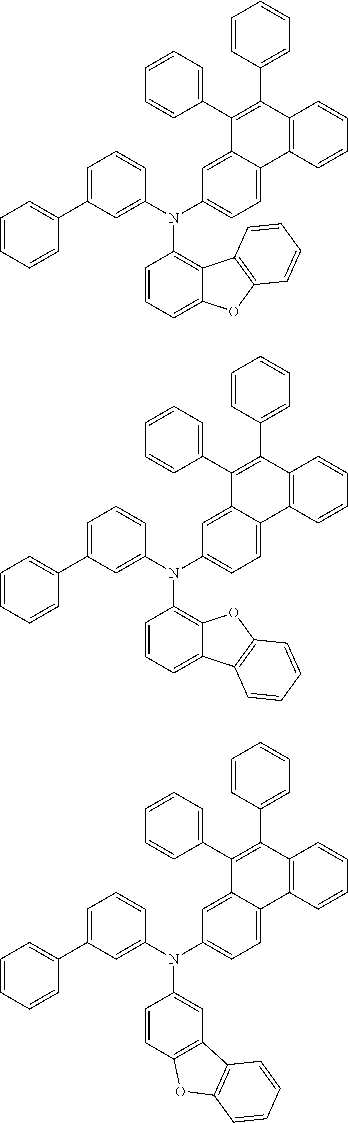 [SMILES: c1ccc(-c2cccc(N(c3ccc4c(c3)c(-c3ccccc3)c(-c3ccccc3)c3ccccc34)c3cccc4oc5ccccc5c34)c2)cc1 ; c1ccc(-c2cccc(N(c3ccc4c(c3)c(-c3ccccc3)c(-c3ccccc3)c3ccccc34)c3cccc4c3oc3ccccc34)c2)cc1 ; c1ccc(-c2cccc(N(c3ccc4c(c3)c(-c3ccccc3)c(-c3ccccc3)c3ccccc34)c3ccc4oc5ccccc5c4c3)c2)cc1]

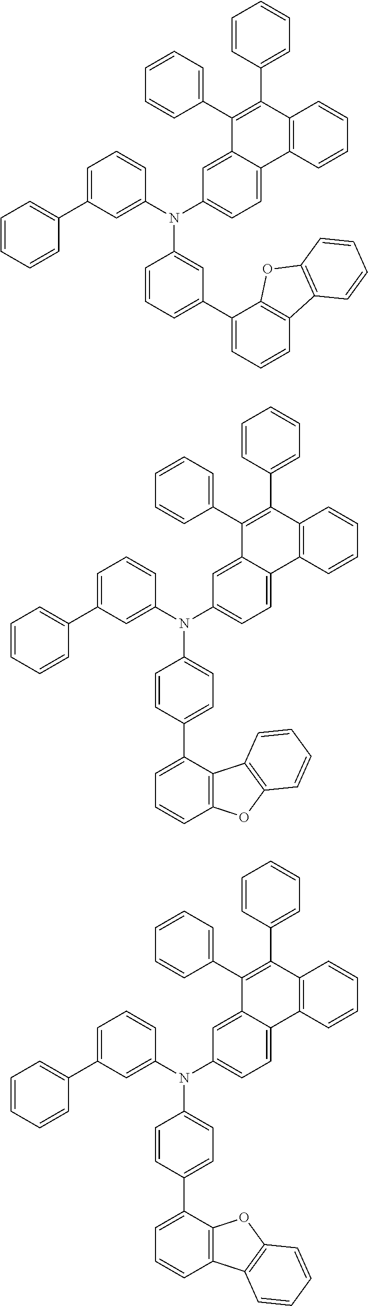

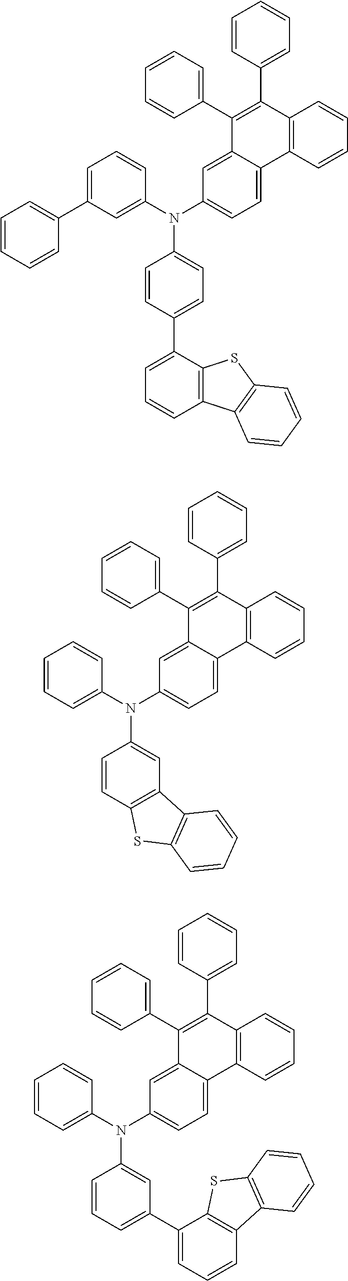

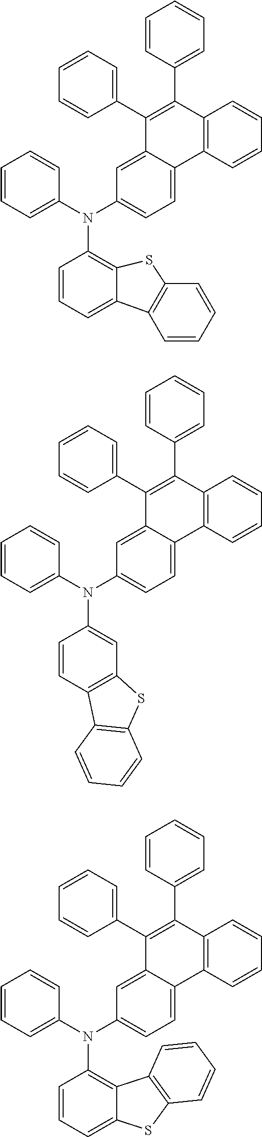

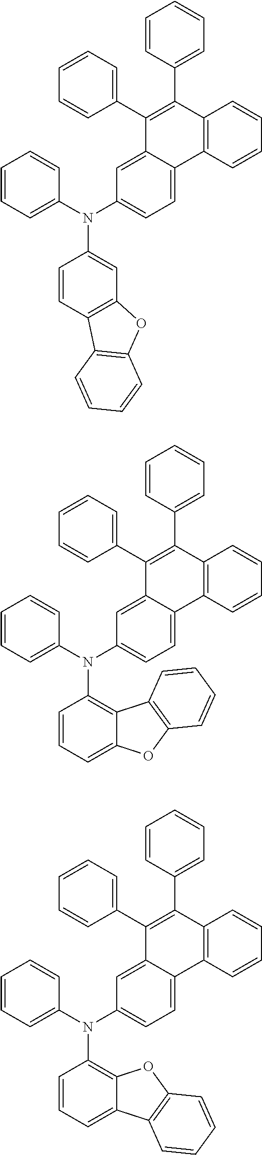 [SMILES: c1ccc(-c2c(-c3ccccc3)c3cc(N(c4ccccc4)c4ccc5c(c4)oc4ccccc45)ccc3c3ccccc23)cc1 ; c1ccc(-c2c(-c3ccccc3)c3cc(N(c4ccccc4)c4cccc5oc6ccccc6c45)ccc3c3ccccc23)cc1 ; c1ccc(-c2c(-c3ccccc3)c3cc(N(c4ccccc4)c4cccc5c4oc4ccccc45)ccc3c3ccccc23)cc1]

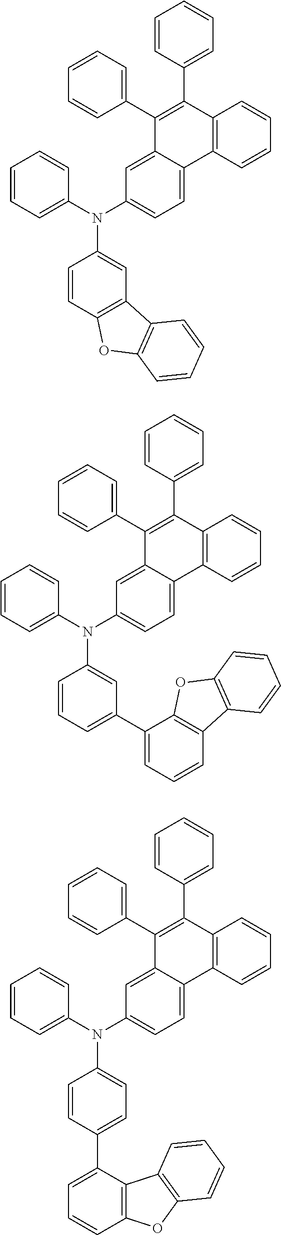

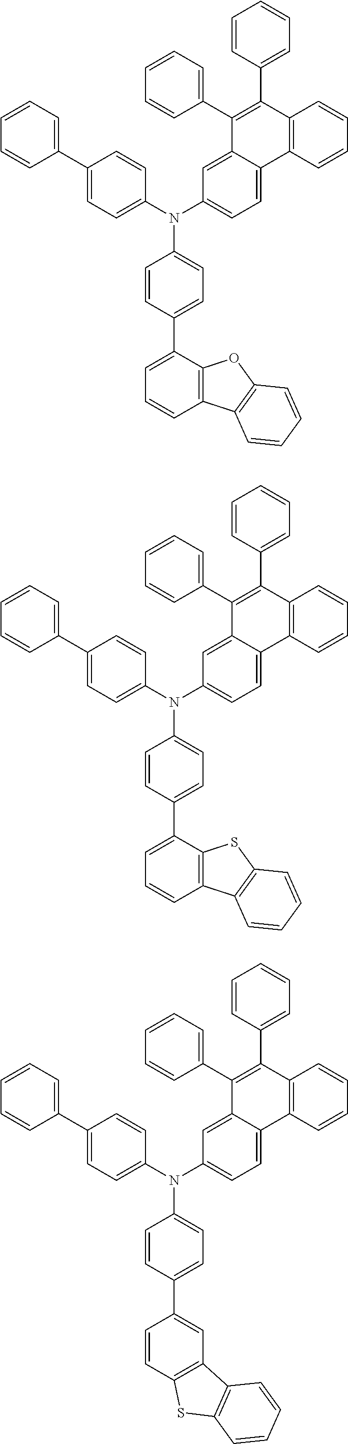

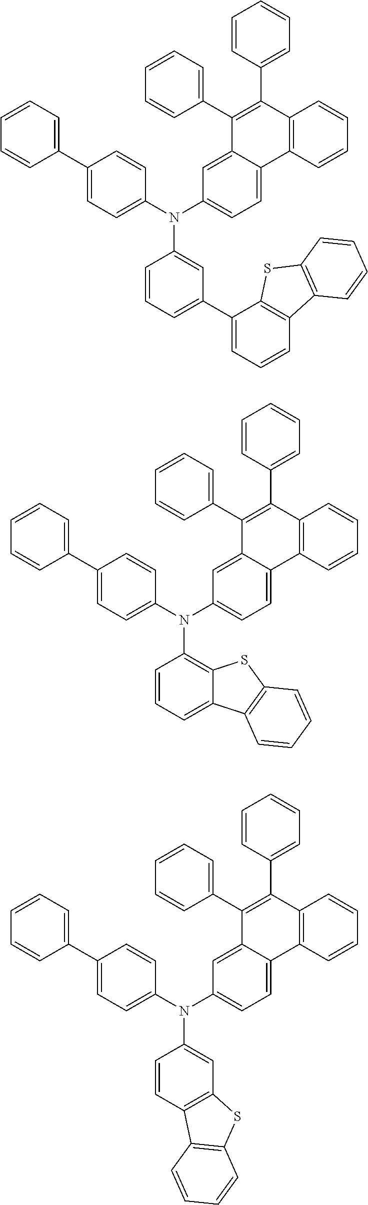

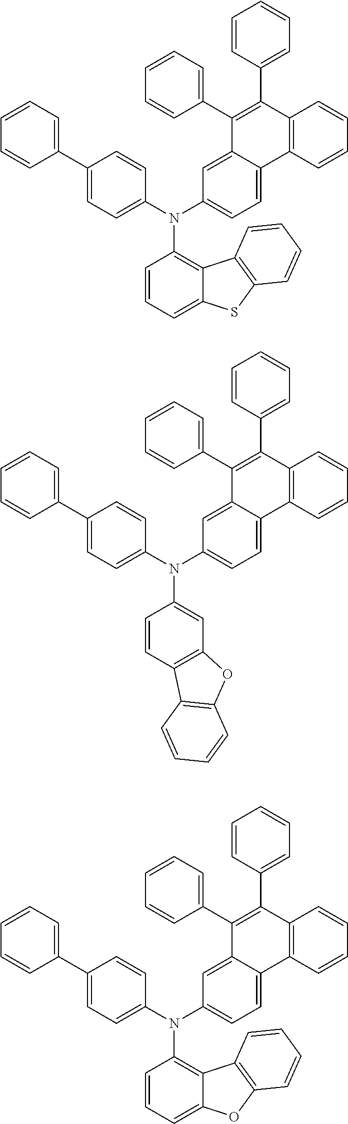 [SMILES: c1ccc(-c2ccc(N(c3ccc4c(c3)c(-c3ccccc3)c(-c3ccccc3)c3ccccc34)c3cccc4sc5ccccc5c34)cc2)cc1 ; c1ccc(-c2ccc(N(c3ccc4c(c3)oc3ccccc34)c3ccc4c(c3)c(-c3ccccc3)c(-c3ccccc3)c3ccccc34)cc2)cc1 ; c1ccc(-c2ccc(N(c3ccc4c(c3)c(-c3ccccc3)c(-c3ccccc3)c3ccccc34)c3cccc4oc5ccccc5c34)cc2)cc1]

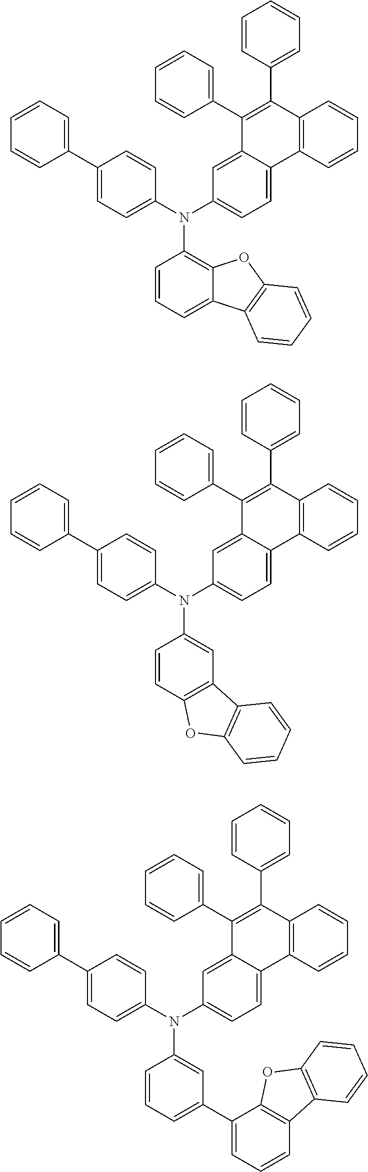

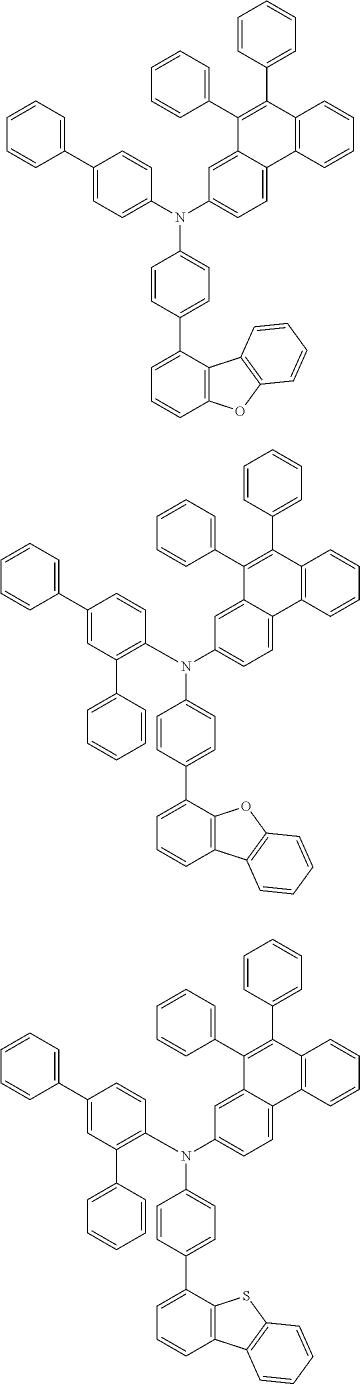 [SMILES: c1ccc(-c2ccc(N(c3ccc(-c4cccc5oc6ccccc6c45)cc3)c3ccc4c(c3)c(-c3ccccc3)c(-c3ccccc3)c3ccccc34)cc2)cc1 ; c1ccc(-c2ccc(N(c3ccc(-c4cccc5c4oc4ccccc45)cc3)c3ccc4c(c3)c(-c3ccccc3)c(-c3ccccc3)c3ccccc34)c(-c3ccccc3)c2)cc1 ; c1ccc(-c2ccc(N(c3ccc(-c4cccc5c4sc4ccccc45)cc3)c3ccc4c(c3)c(-c3ccccc3)c(-c3ccccc3)c3ccccc34)c(-c3ccccc3)c2)cc1]

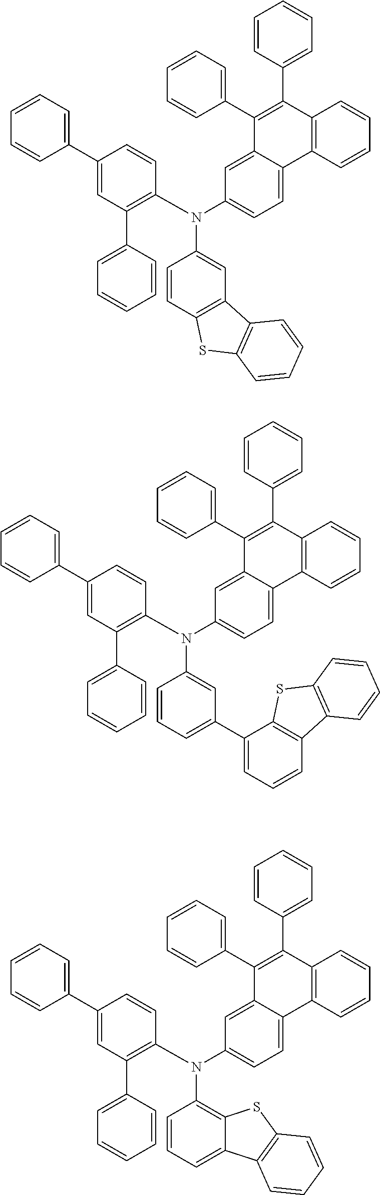

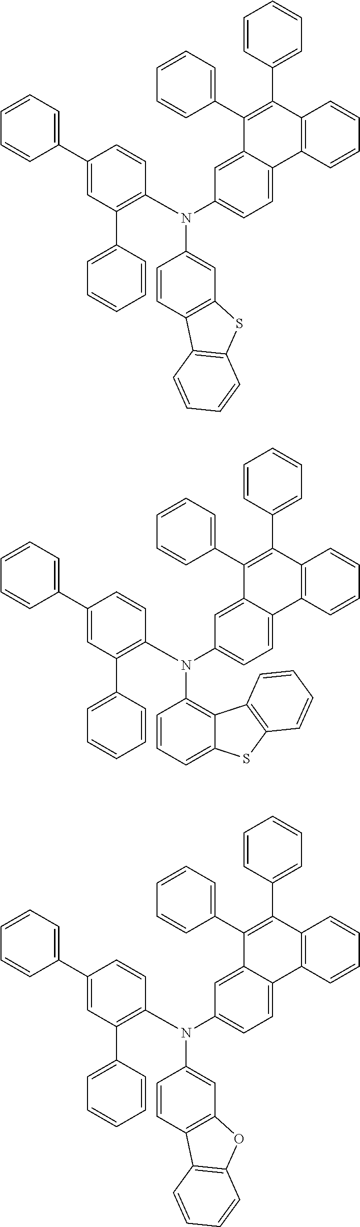 [SMILES: c1ccc(-c2ccc(N(c3ccc4c(c3)sc3ccccc34)c3ccc4c(c3)c(-c3ccccc3)c(-c3ccccc3)c3ccccc34)c(-c3ccccc3)c2)cc1 ; c1ccc(-c2ccc(N(c3ccc4c(c3)c(-c3ccccc3)c(-c3ccccc3)c3ccccc34)c3cccc4sc5ccccc5c34)c(-c3ccccc3)c2)cc1 ; c1ccc(-c2ccc(N(c3ccc4c(c3)oc3ccccc34)c3ccc4c(c3)c(-c3ccccc3)c(-c3ccccc3)c3ccccc34)c(-c3ccccc3)c2)cc1]

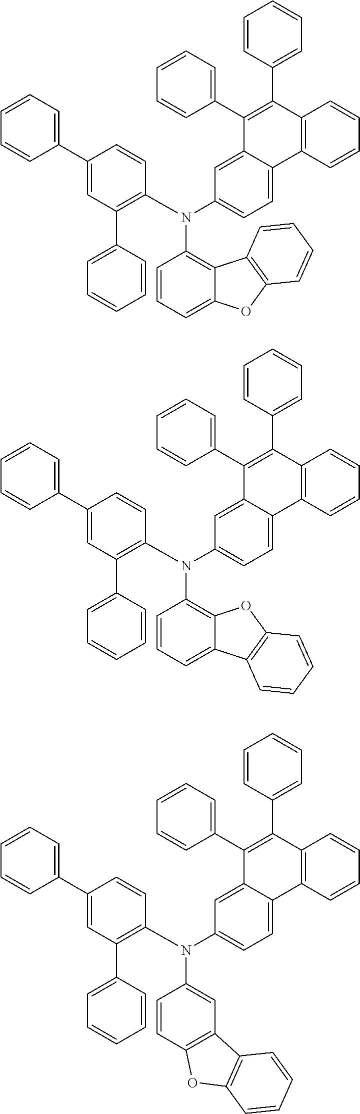

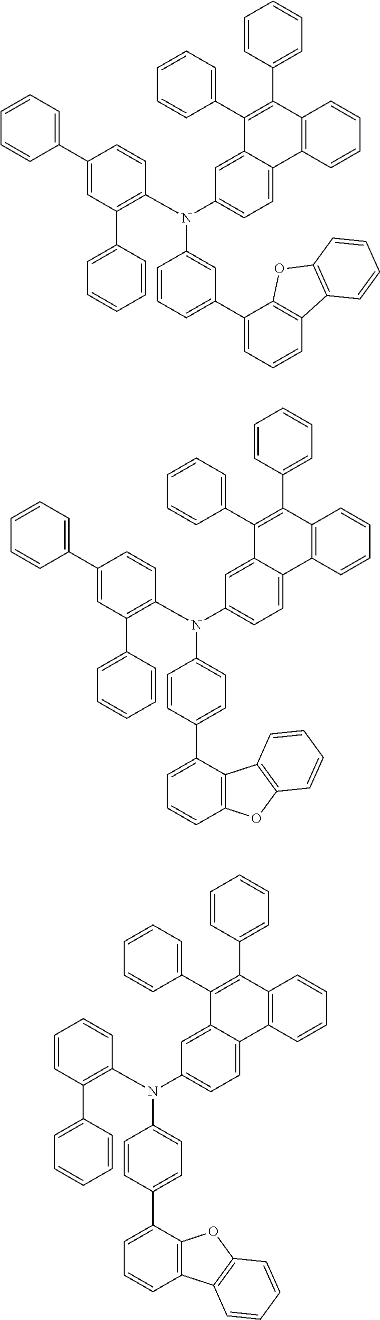

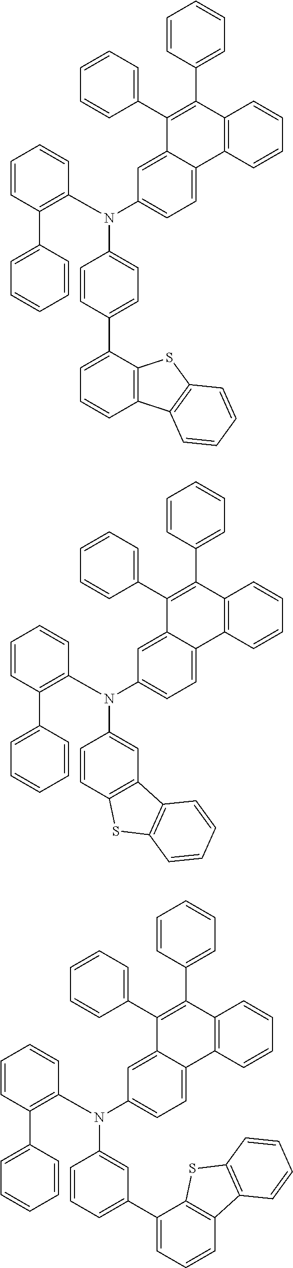

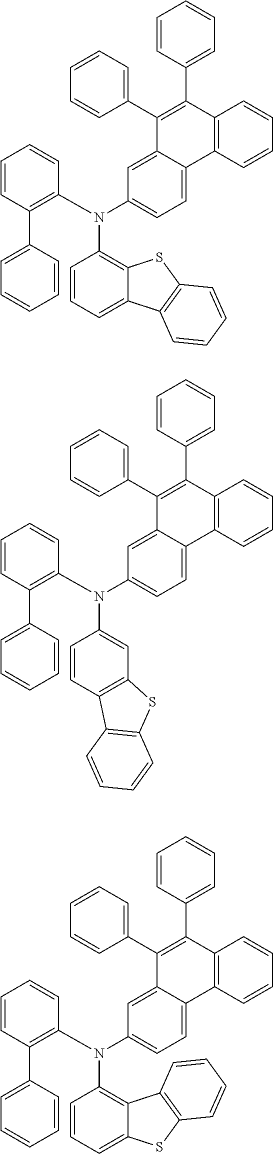

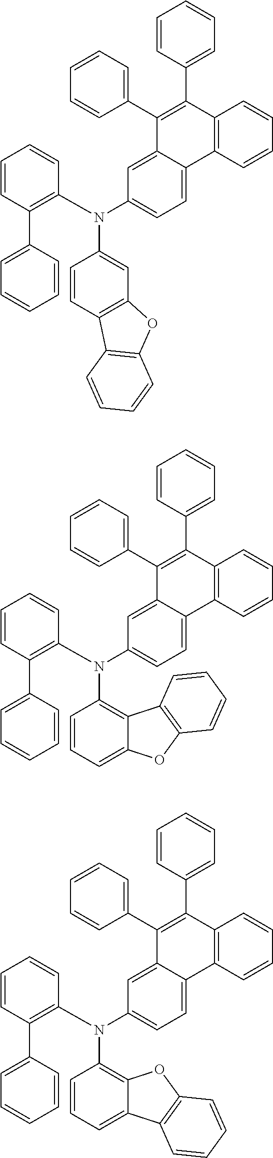 [SMILES: c1ccc(-c2ccccc2N(c2ccc3c(c2)oc2ccccc23)c2ccc3c(c2)c(-c2ccccc2)c(-c2ccccc2)c2ccccc23)cc1 ; c1ccc(-c2ccccc2N(c2ccc3c(c2)c(-c2ccccc2)c(-c2ccccc2)c2ccccc23)c2cccc3oc4ccccc4c23)cc1 ; c1ccc(-c2ccccc2N(c2ccc3c(c2)c(-c2ccccc2)c(-c2ccccc2)c2ccccc23)c2cccc3c2oc2ccccc23)cc1]

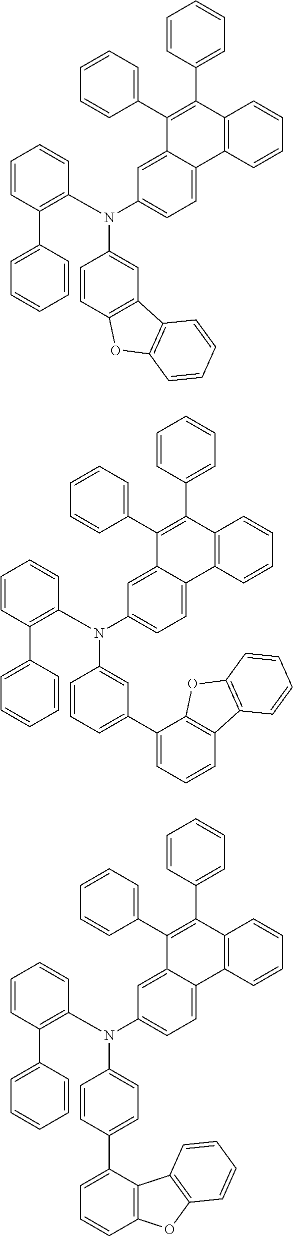

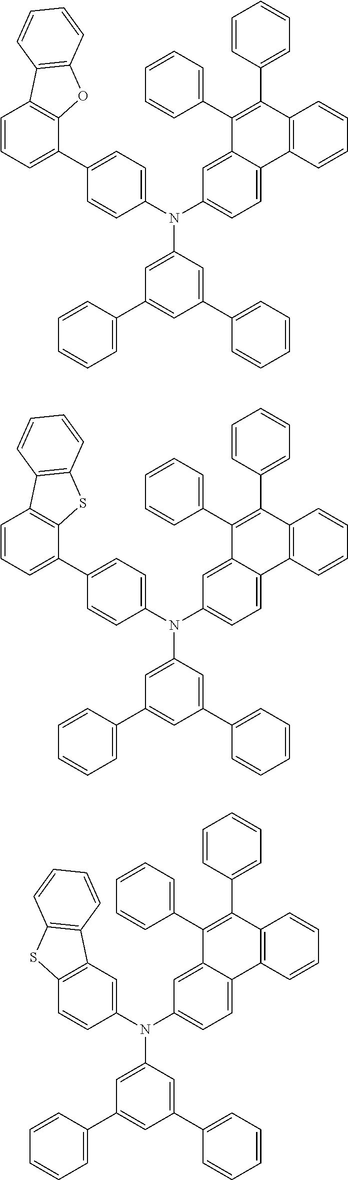

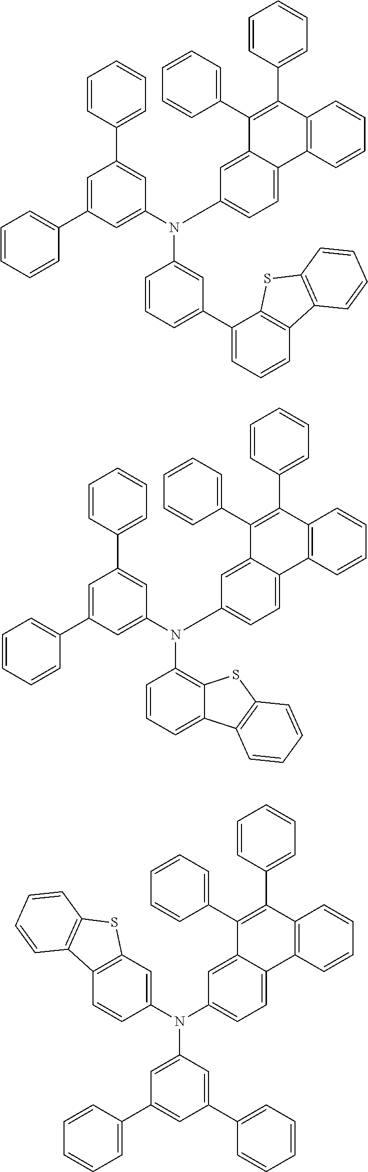

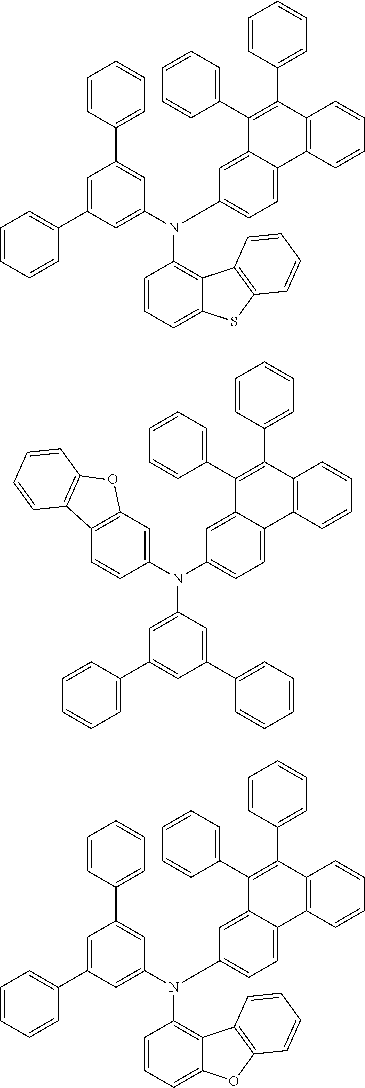

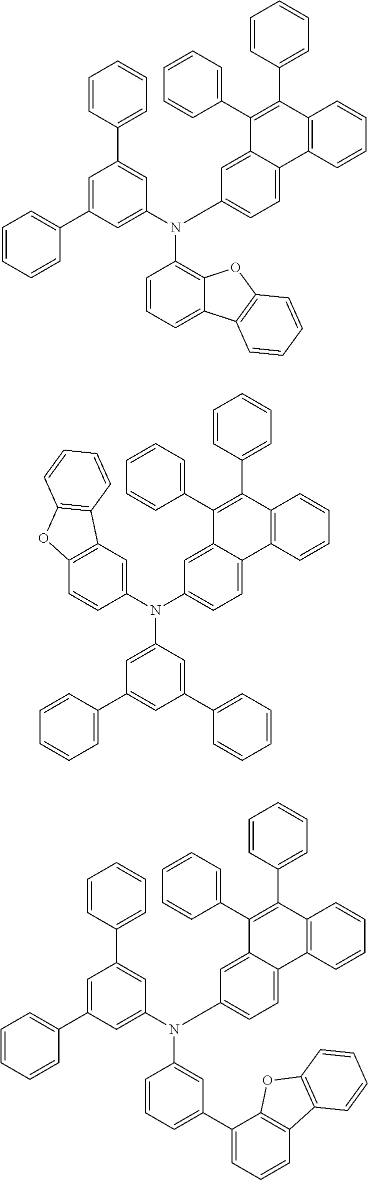

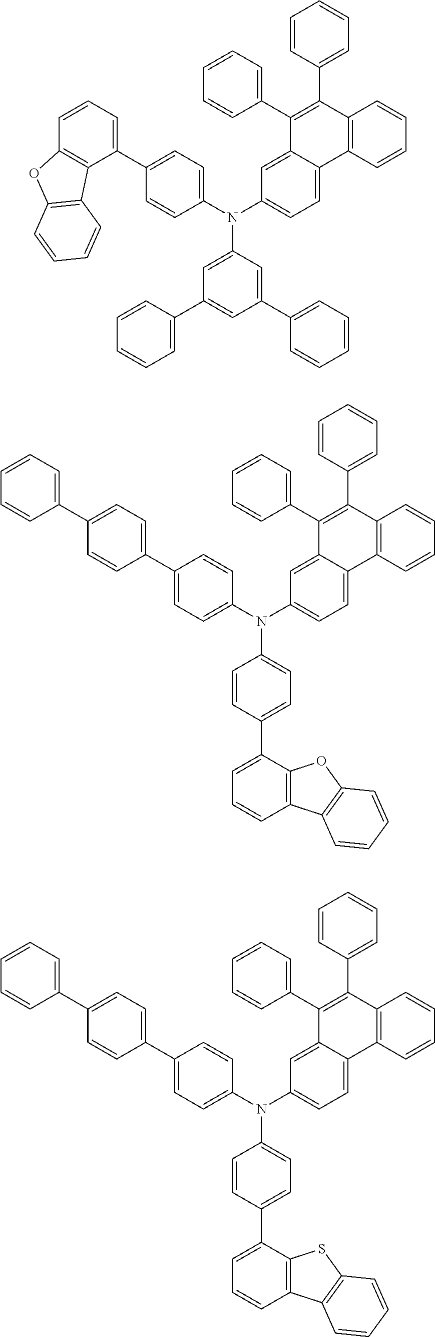 [SMILES: c1ccc(-c2cc(-c3ccccc3)cc(N(c3ccc(-c4cccc5oc6ccccc6c45)cc3)c3ccc4c(c3)c(-c3ccccc3)c(-c3ccccc3)c3ccccc34)c2)cc1 ; c1ccc(-c2ccc(-c3ccc(N(c4ccc(-c5cccc6c5oc5ccccc56)cc4)c4ccc5c(c4)c(-c4ccccc4)c(-c4ccccc4)c4ccccc45)cc3)cc2)cc1 ; c1ccc(-c2ccc(-c3ccc(N(c4ccc(-c5cccc6c5sc5ccccc56)cc4)c4ccc5c(c4)c(-c4ccccc4)c(-c4ccccc4)c4ccccc45)cc3)cc2)cc1]

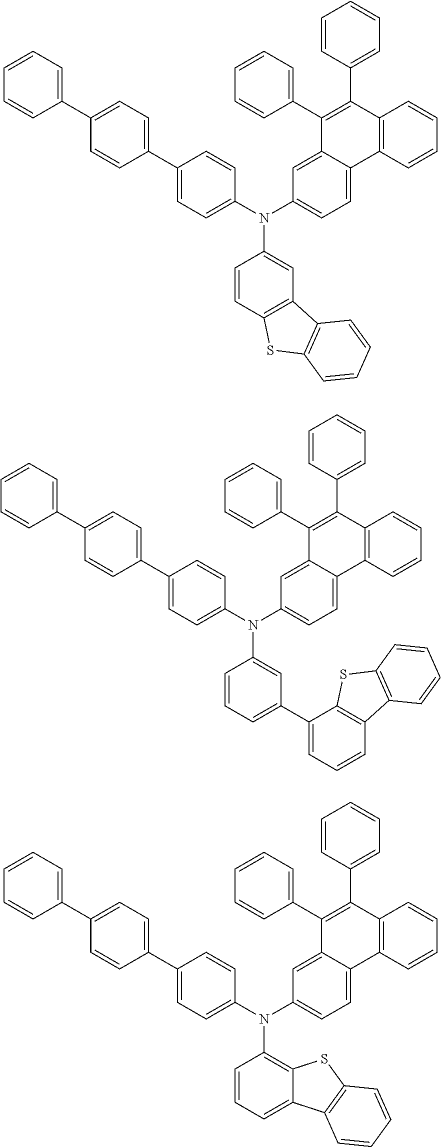

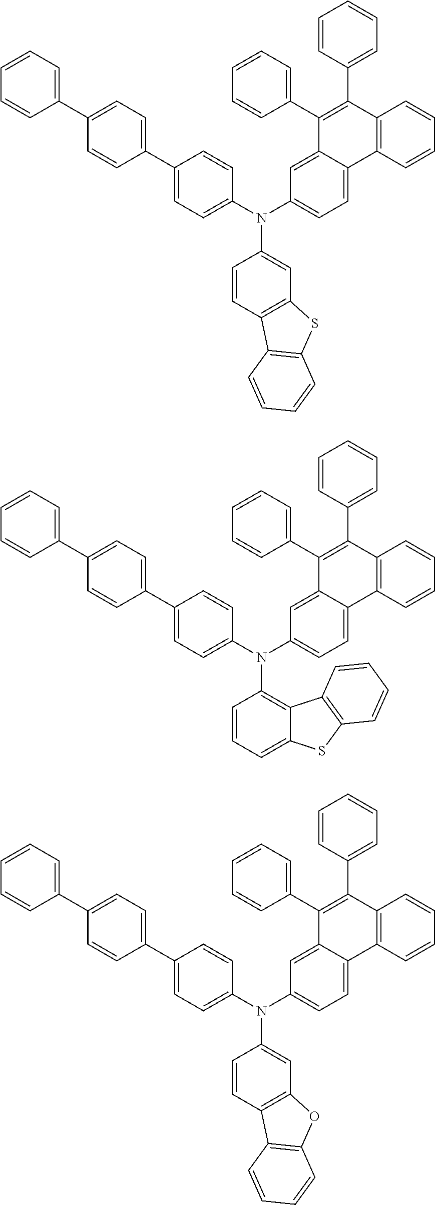 [SMILES: c1ccc(-c2ccc(-c3ccc(N(c4ccc5c(c4)sc4ccccc45)c4ccc5c(c4)c(-c4ccccc4)c(-c4ccccc4)c4ccccc45)cc3)cc2)cc1 ; c1ccc(-c2ccc(-c3ccc(N(c4ccc5c(c4)c(-c4ccccc4)c(-c4ccccc4)c4ccccc45)c4cccc5sc6ccccc6c45)cc3)cc2)cc1 ; c1ccc(-c2ccc(-c3ccc(N(c4ccc5c(c4)oc4ccccc45)c4ccc5c(c4)c(-c4ccccc4)c(-c4ccccc4)c4ccccc45)cc3)cc2)cc1]

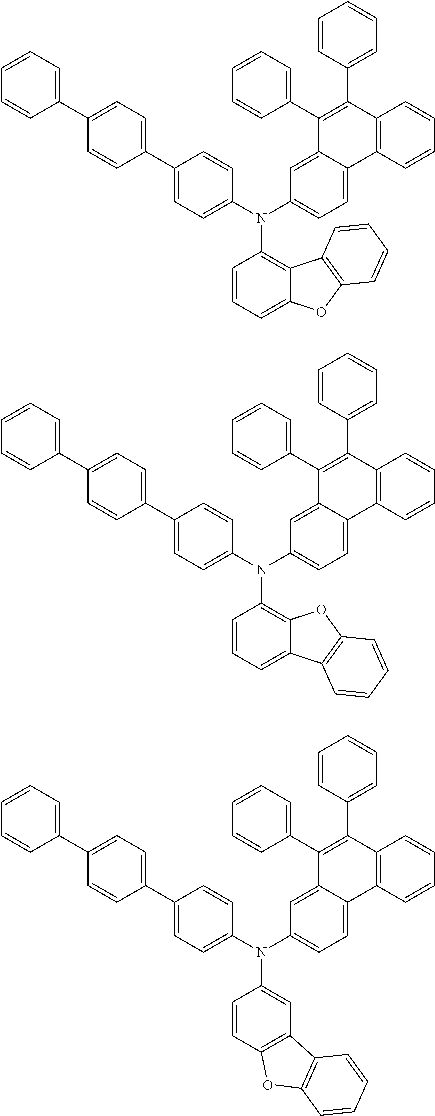

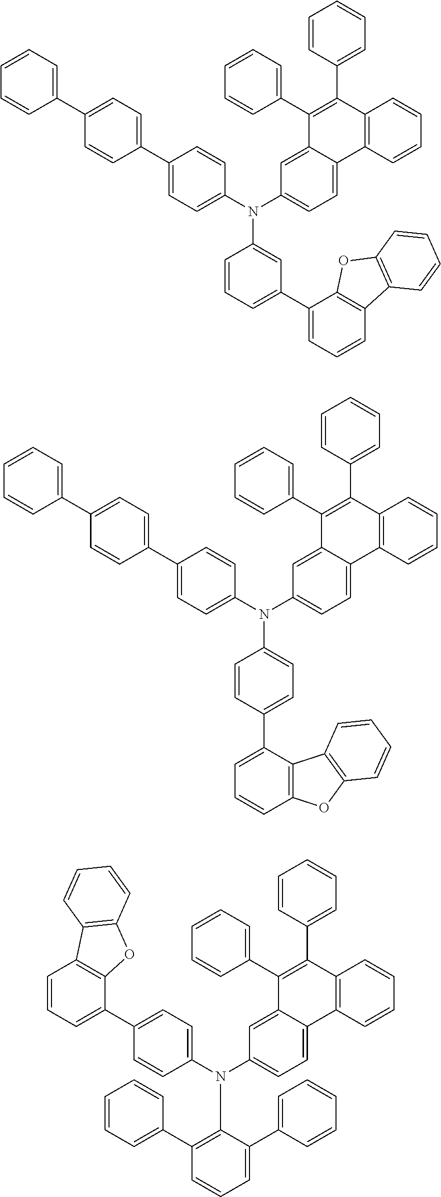

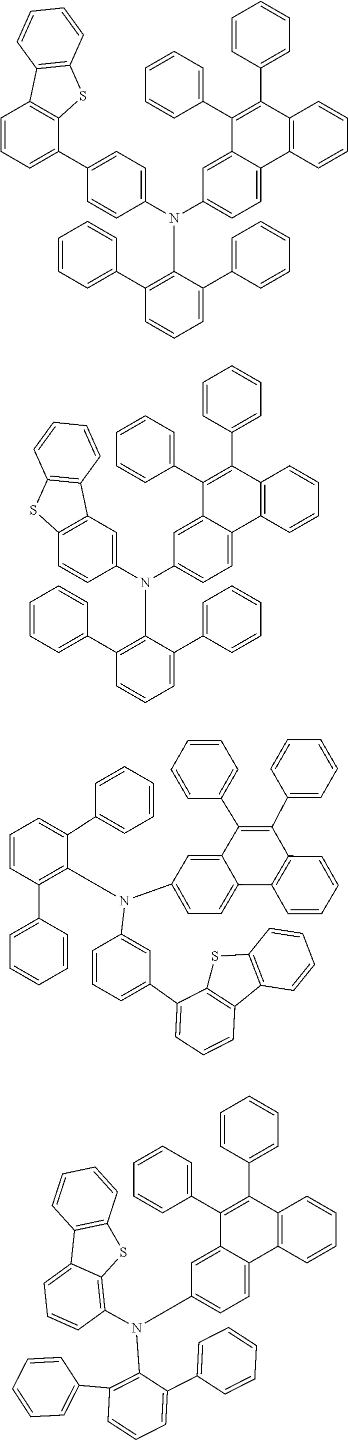 [SMILES: c1ccc(-c2cccc(-c3ccccc3)c2N(c2ccc(-c3cccc4c3sc3ccccc34)cc2)c2ccc3c(c2)c(-c2ccccc2)c(-c2ccccc2)c2ccccc23)cc1 ; c1ccc(-c2cccc(-c3ccccc3)c2N(c2ccc3c(c2)c(-c2ccccc2)c(-c2ccccc2)c2ccccc23)c2ccc3sc4ccccc4c3c2)cc1 ; c1ccc(-c2cccc(-c3ccccc3)c2N(c2cccc(-c3cccc4c3sc3ccccc34)c2)c2ccc3c(c2)c(-c2ccccc2)c(-c2ccccc2)c2ccccc23)cc1 ; c1ccc(-c2cccc(-c3ccccc3)c2N(c2ccc3c(c2)c(-c2ccccc2)c(-c2ccccc2)c2ccccc23)c2cccc3c2sc2ccccc23)cc1]

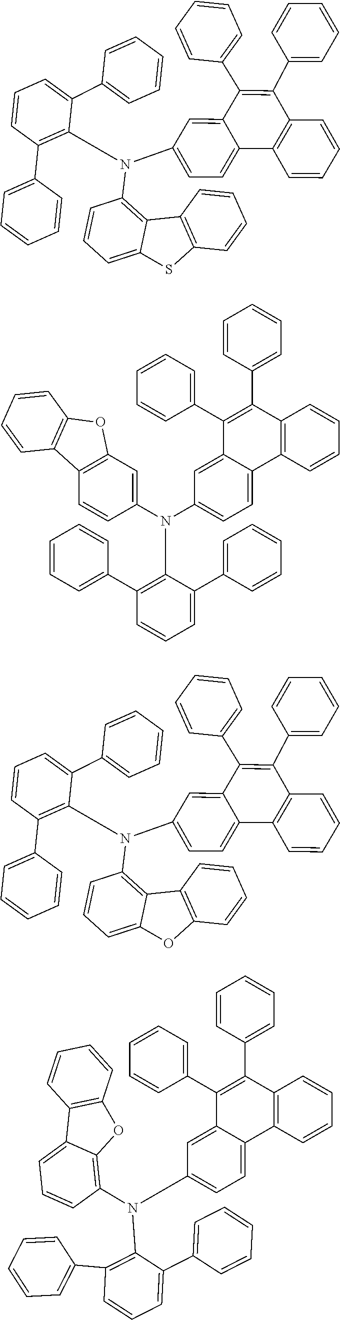

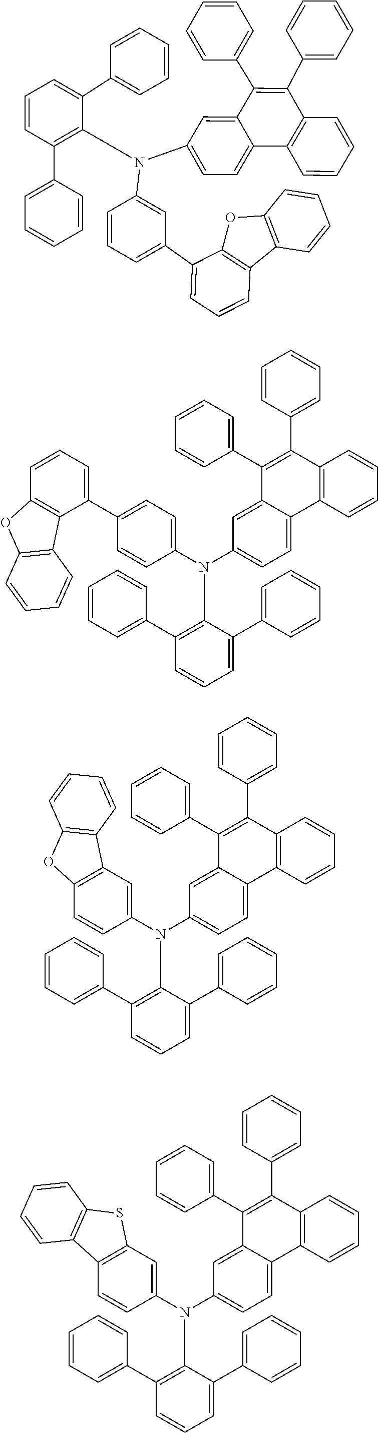 [SMILES: c1ccc(-c2cccc(-c3ccccc3)c2N(c2cccc(-c3cccc4c3oc3ccccc34)c2)c2ccc3c(c2)c(-c2ccccc2)c(-c2ccccc2)c2ccccc23)cc1 ; c1ccc(-c2cccc(-c3ccccc3)c2N(c2ccc(-c3cccc4oc5ccccc5c34)cc2)c2ccc3c(c2)c(-c2ccccc2)c(-c2ccccc2)c2ccccc23)cc1 ; c1ccc(-c2cccc(-c3ccccc3)c2N(c2ccc3c(c2)c(-c2ccccc2)c(-c2ccccc2)c2ccccc23)c2ccc3oc4ccccc4c3c2)cc1 ; c1ccc(-c2cccc(-c3ccccc3)c2N(c2ccc3c(c2)sc2ccccc23)c2ccc3c(c2)c(-c2ccccc2)c(-c2ccccc2)c2ccccc23)cc1]

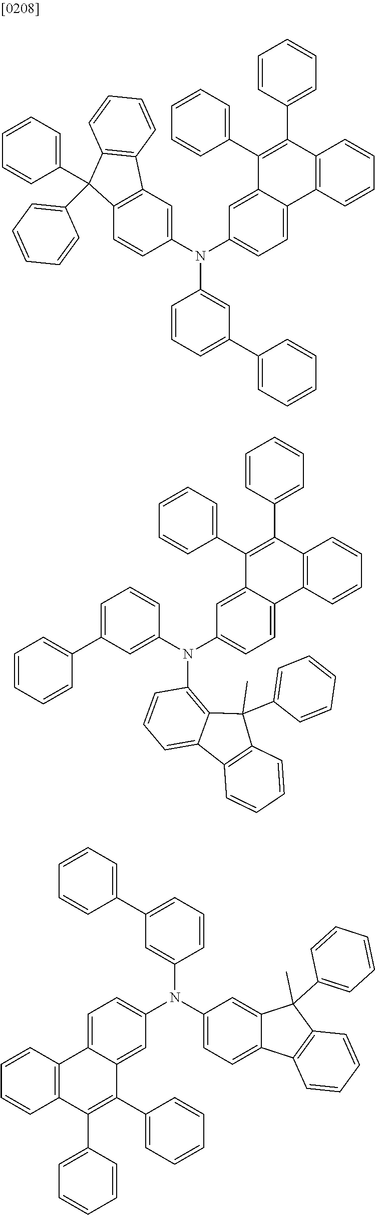

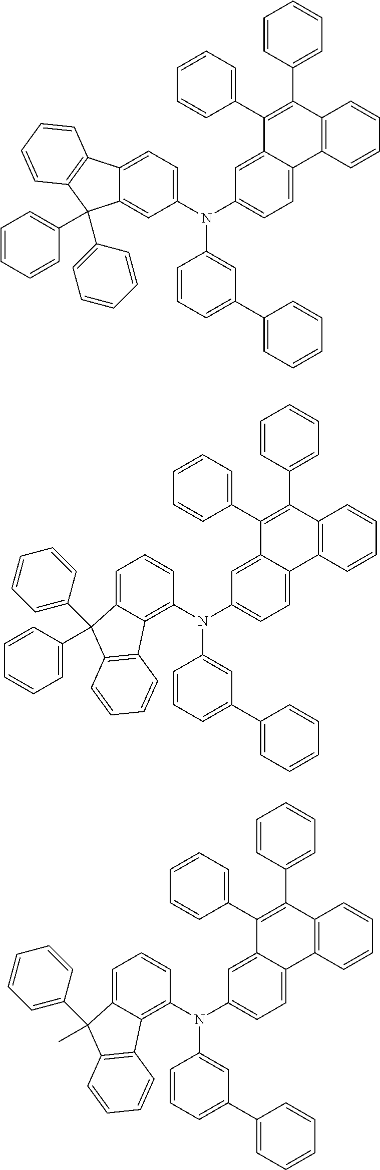

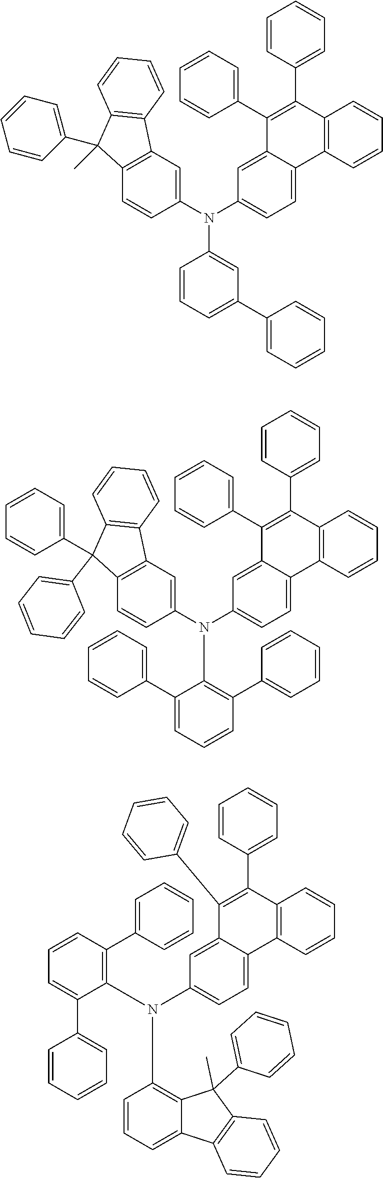

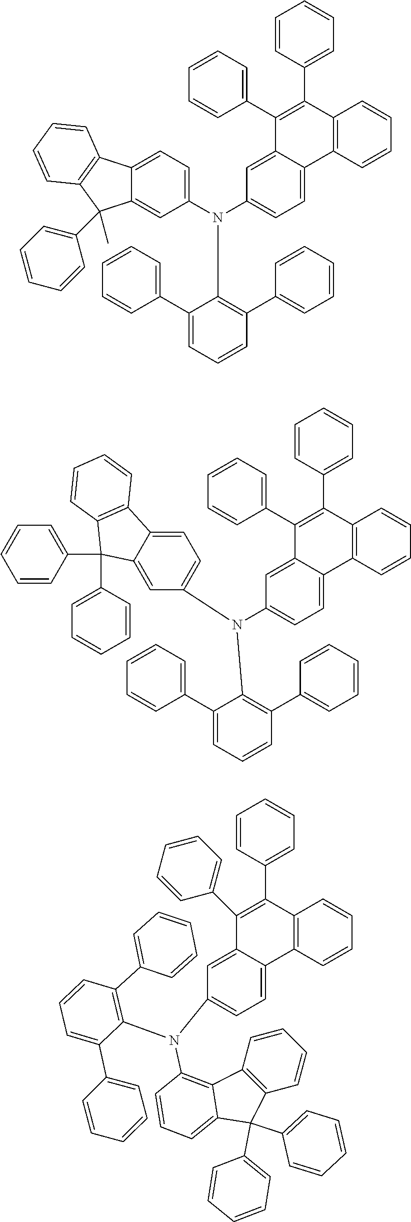

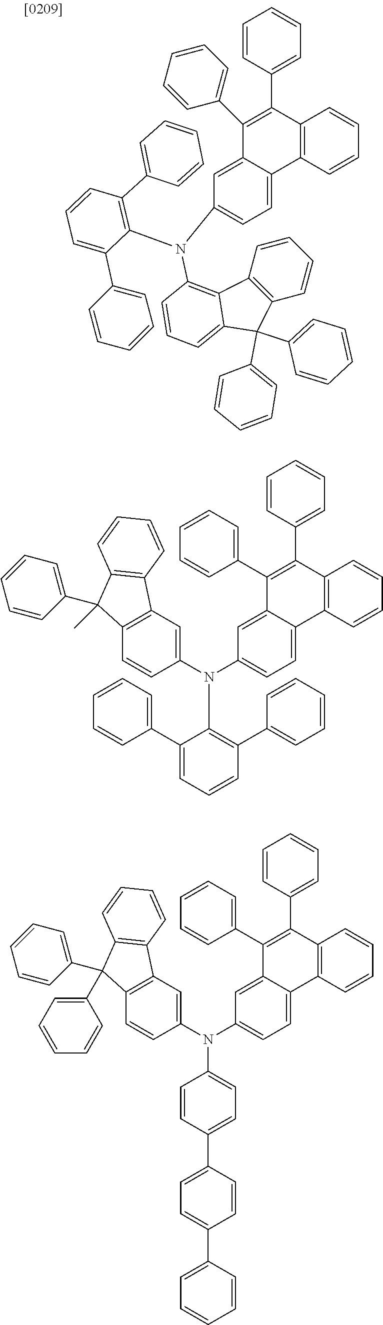 [SMILES: c1ccc(-c2cccc(-c3ccccc3)c2N(c2ccc3c(c2)c(-c2ccccc2)c(-c2ccccc2)c2ccccc23)c2cccc3c2-c2ccccc2C3(c2ccccc2)c2ccccc2)cc1 ; CC1(c2ccccc2)c2ccccc2-c2cc(N(c3ccc4c(c3)c(-c3ccccc3)c(-c3ccccc3)c3ccccc34)c3c(-c4ccccc4)cccc3-c3ccccc3)ccc21 ; c1ccc(-c2ccc(-c3ccc(N(c4ccc5c(c4)-c4ccccc4C5(c4ccccc4)c4ccccc4)c4ccc5c(c4)c(-c4ccccc4)c(-c4ccccc4)c4ccccc45)cc3)cc2)cc1]

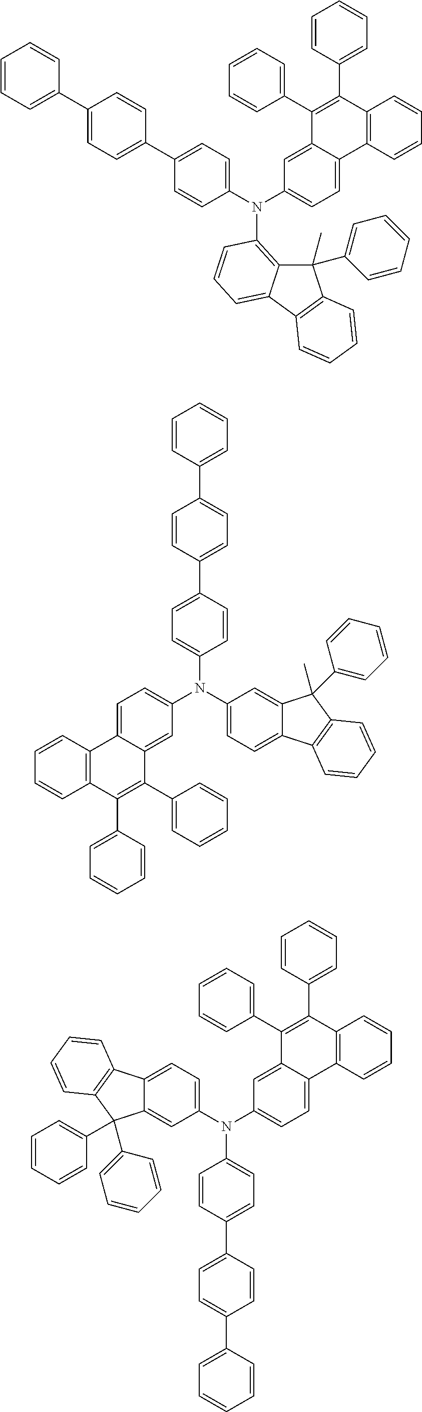 [SMILES: CC1(c2ccccc2)c2ccccc2-c2cccc(N(c3ccc(-c4ccc(-c5ccccc5)cc4)cc3)c3ccc4c(c3)c(-c3ccccc3)c(-c3ccccc3)c3ccccc34)c21 ; CC1(c2ccccc2)c2ccccc2-c2ccc(N(c3ccc(-c4ccc(-c5ccccc5)cc4)cc3)c3ccc4c(c3)c(-c3ccccc3)c(-c3ccccc3)c3ccccc34)cc21 ; c1ccc(-c2ccc(-c3ccc(N(c4ccc5c(c4)C(c4ccccc4)(c4ccccc4)c4ccccc4-5)c4ccc5c(c4)c(-c4ccccc4)c(-c4ccccc4)c4ccccc45)cc3)cc2)cc1]

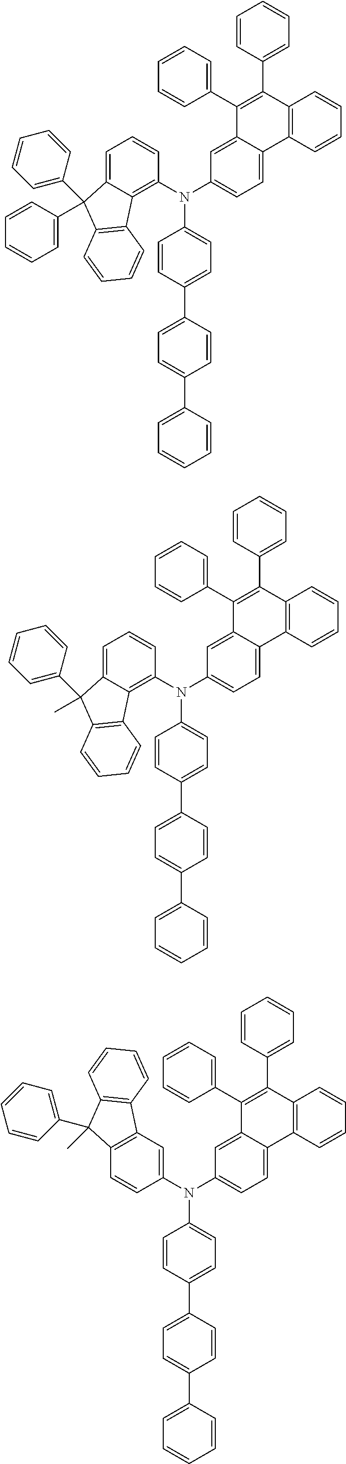 [SMILES: c1ccc(-c2ccc(-c3ccc(N(c4ccc5c(c4)c(-c4ccccc4)c(-c4ccccc4)c4ccccc45)c4cccc5c4-c4ccccc4C5(c4ccccc4)c4ccccc4)cc3)cc2)cc1 ; CC1(c2ccccc2)c2ccccc2-c2c(N(c3ccc(-c4ccc(-c5ccccc5)cc4)cc3)c3ccc4c(c3)c(-c3ccccc3)c(-c3ccccc3)c3ccccc34)cccc21 ; CC1(c2ccccc2)c2ccccc2-c2cc(N(c3ccc(-c4ccc(-c5ccccc5)cc4)cc3)c3ccc4c(c3)c(-c3ccccc3)c(-c3ccccc3)c3ccccc34)ccc21]

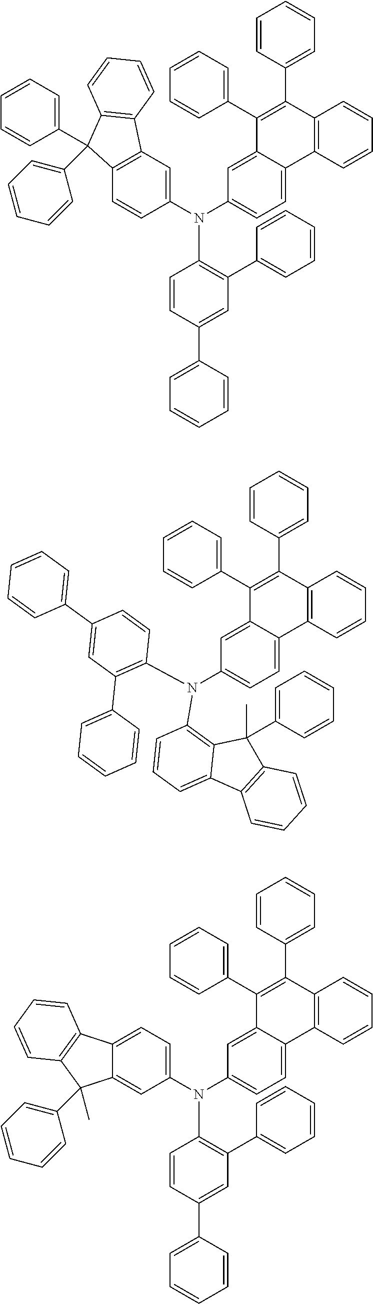 [SMILES: c1ccc(-c2ccc(N(c3ccc4c(c3)-c3ccccc3C4(c3ccccc3)c3ccccc3)c3ccc4c(c3)c(-c3ccccc3)c(-c3ccccc3)c3ccccc34)c(-c3ccccc3)c2)cc1 ; CC1(c2ccccc2)c2ccccc2-c2cccc(N(c3ccc4c(c3)c(-c3ccccc3)c(-c3ccccc3)c3ccccc34)c3ccc(-c4ccccc4)cc3-c3ccccc3)c21 ; CC1(c2ccccc2)c2ccccc2-c2ccc(N(c3ccc4c(c3)c(-c3ccccc3)c(-c3ccccc3)c3ccccc34)c3ccc(-c4ccccc4)cc3-c3ccccc3)cc21]

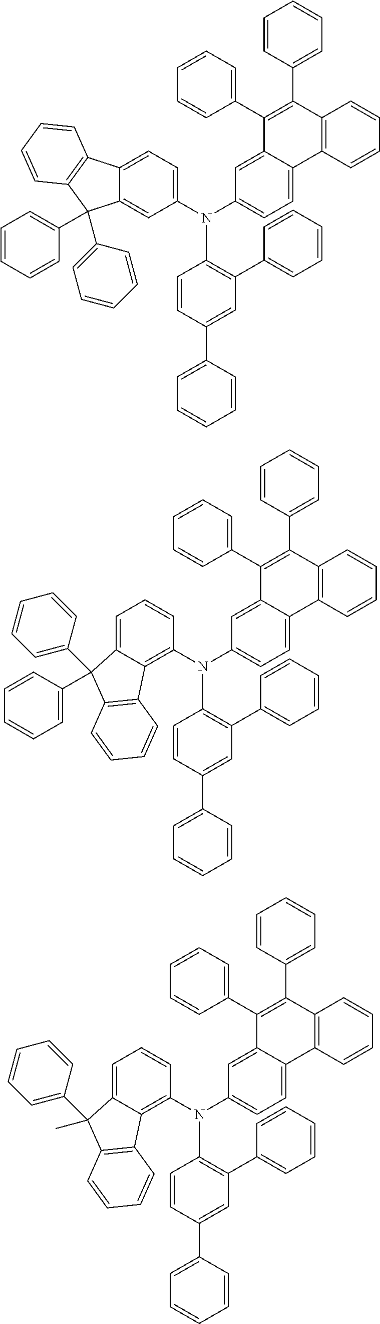 [SMILES: c1ccc(-c2ccc(N(c3ccc4c(c3)C(c3ccccc3)(c3ccccc3)c3ccccc3-4)c3ccc4c(c3)c(-c3ccccc3)c(-c3ccccc3)c3ccccc34)c(-c3ccccc3)c2)cc1 ; c1ccc(-c2ccc(N(c3ccc4c(c3)c(-c3ccccc3)c(-c3ccccc3)c3ccccc34)c3cccc4c3-c3ccccc3C4(c3ccccc3)c3ccccc3)c(-c3ccccc3)c2)cc1 ; CC1(c2ccccc2)c2ccccc2-c2c(N(c3ccc4c(c3)c(-c3ccccc3)c(-c3ccccc3)c3ccccc34)c3ccc(-c4ccccc4)cc3-c3ccccc3)cccc21]

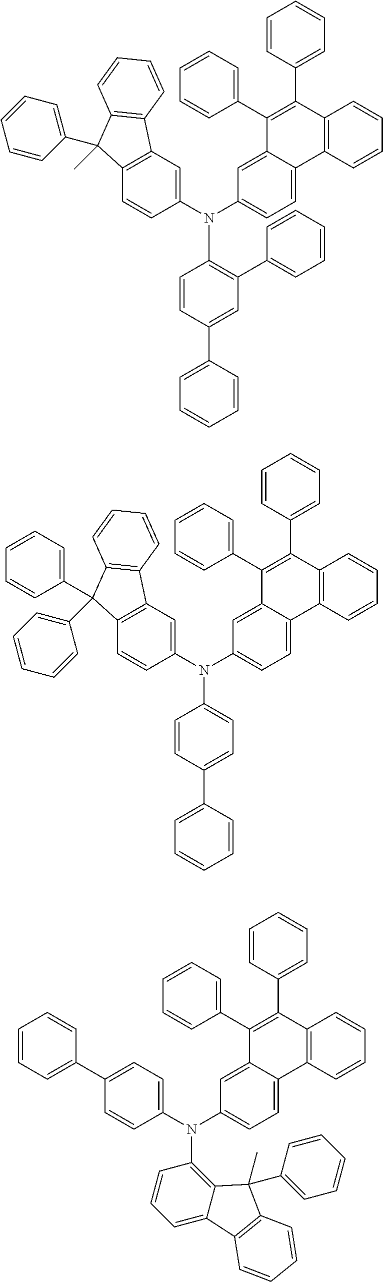 [SMILES: CC1(c2ccccc2)c2ccccc2-c2cc(N(c3ccc4c(c3)c(-c3ccccc3)c(-c3ccccc3)c3ccccc34)c3ccc(-c4ccccc4)cc3-c3ccccc3)ccc21 ; c1ccc(-c2ccc(N(c3ccc4c(c3)-c3ccccc3C4(c3ccccc3)c3ccccc3)c3ccc4c(c3)c(-c3ccccc3)c(-c3ccccc3)c3ccccc34)cc2)cc1 ; CC1(c2ccccc2)c2ccccc2-c2cccc(N(c3ccc(-c4ccccc4)cc3)c3ccc4c(c3)c(-c3ccccc3)c(-c3ccccc3)c3ccccc34)c21]

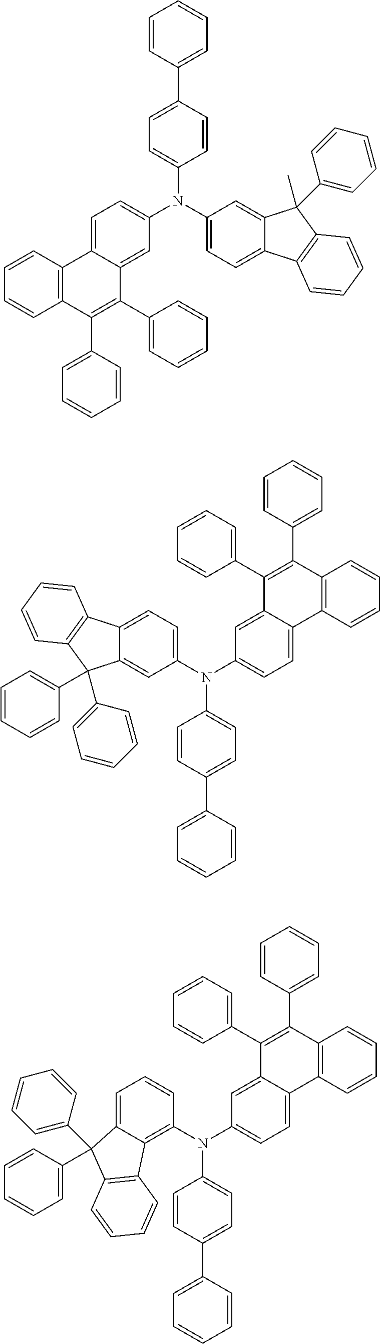 [SMILES: CC1(c2ccccc2)c2ccccc2-c2ccc(N(c3ccc(-c4ccccc4)cc3)c3ccc4c(c3)c(-c3ccccc3)c(-c3ccccc3)c3ccccc34)cc21 ; c1ccc(-c2ccc(N(c3ccc4c(c3)C(c3ccccc3)(c3ccccc3)c3ccccc3-4)c3ccc4c(c3)c(-c3ccccc3)c(-c3ccccc3)c3ccccc34)cc2)cc1 ; c1ccc(-c2ccc(N(c3ccc4c(c3)c(-c3ccccc3)c(-c3ccccc3)c3ccccc34)c3cccc4c3-c3ccccc3C4(c3ccccc3)c3ccccc3)cc2)cc1]

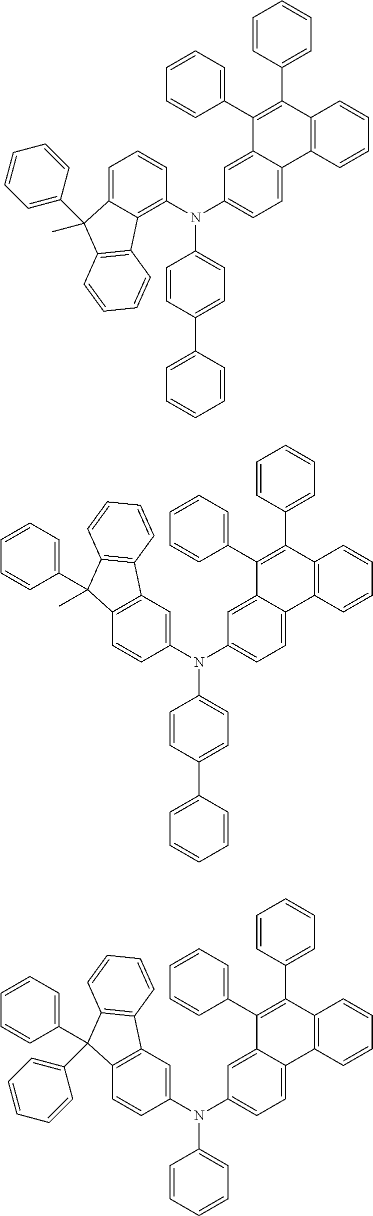 [SMILES: CC1(c2ccccc2)c2ccccc2-c2c(N(c3ccc(-c4ccccc4)cc3)c3ccc4c(c3)c(-c3ccccc3)c(-c3ccccc3)c3ccccc34)cccc21 ; CC1(c2ccccc2)c2ccccc2-c2cc(N(c3ccc(-c4ccccc4)cc3)c3ccc4c(c3)c(-c3ccccc3)c(-c3ccccc3)c3ccccc34)ccc21 ; c1ccc(-c2c(-c3ccccc3)c3cc(N(c4ccccc4)c4ccc5c(c4)-c4ccccc4C5(c4ccccc4)c4ccccc4)ccc3c3ccccc23)cc1]

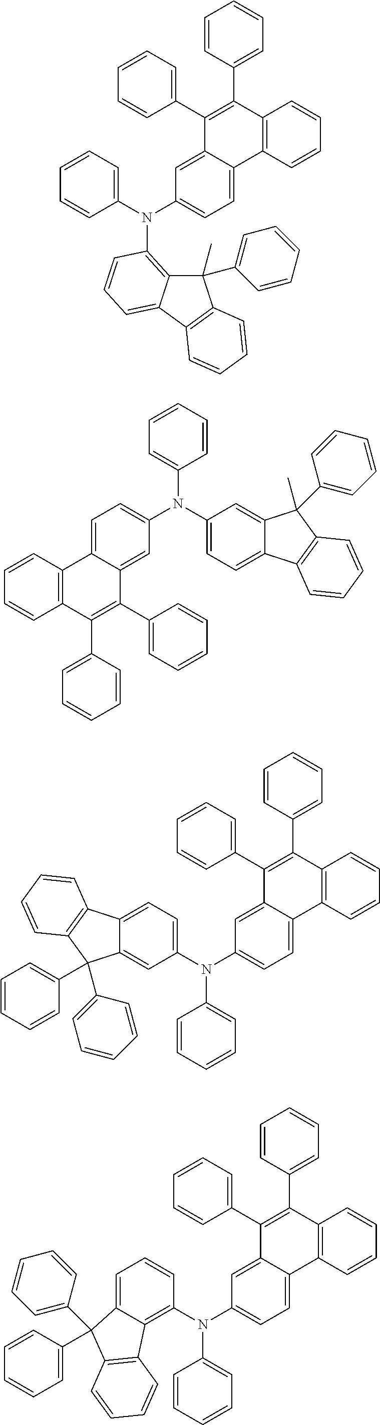

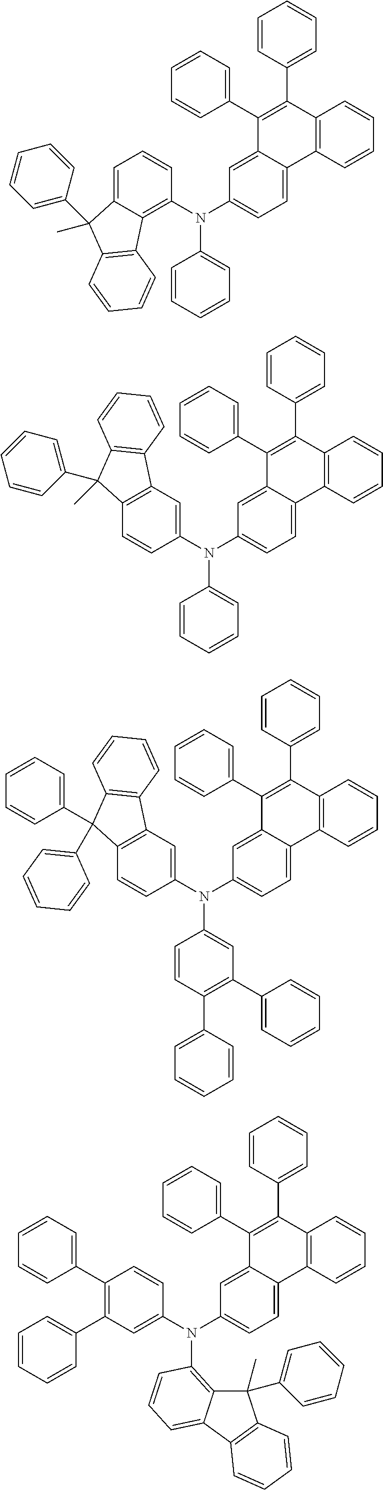 [SMILES: CC1(c2ccccc2)c2ccccc2-c2c(N(c3ccccc3)c3ccc4c(c3)c(-c3ccccc3)c(-c3ccccc3)c3ccccc34)cccc21 ; CC1(c2ccccc2)c2ccccc2-c2cc(N(c3ccccc3)c3ccc4c(c3)c(-c3ccccc3)c(-c3ccccc3)c3ccccc34)ccc21 ; c1ccc(-c2ccc(N(c3ccc4c(c3)-c3ccccc3C4(c3ccccc3)c3ccccc3)c3ccc4c(c3)c(-c3ccccc3)c(-c3ccccc3)c3ccccc34)cc2-c2ccccc2)cc1 ; CC1(c2ccccc2)c2ccccc2-c2cccc(N(c3ccc(-c4ccccc4)c(-c4ccccc4)c3)c3ccc4c(c3)c(-c3ccccc3)c(-c3ccccc3)c3ccccc34)c21]

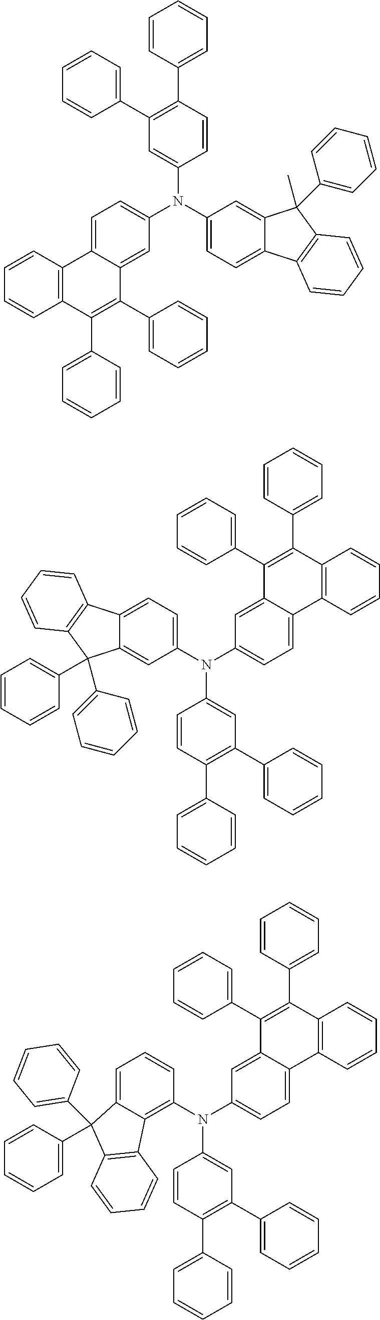 [SMILES: CC1(c2ccccc2)c2ccccc2-c2ccc(N(c3ccc(-c4ccccc4)c(-c4ccccc4)c3)c3ccc4c(c3)c(-c3ccccc3)c(-c3ccccc3)c3ccccc34)cc21 ; c1ccc(-c2ccc(N(c3ccc4c(c3)C(c3ccccc3)(c3ccccc3)c3ccccc3-4)c3ccc4c(c3)c(-c3ccccc3)c(-c3ccccc3)c3ccccc34)cc2-c2ccccc2)cc1 ; c1ccc(-c2ccc(N(c3ccc4c(c3)c(-c3ccccc3)c(-c3ccccc3)c3ccccc34)c3cccc4c3-c3ccccc3C4(c3ccccc3)c3ccccc3)cc2-c2ccccc2)cc1]

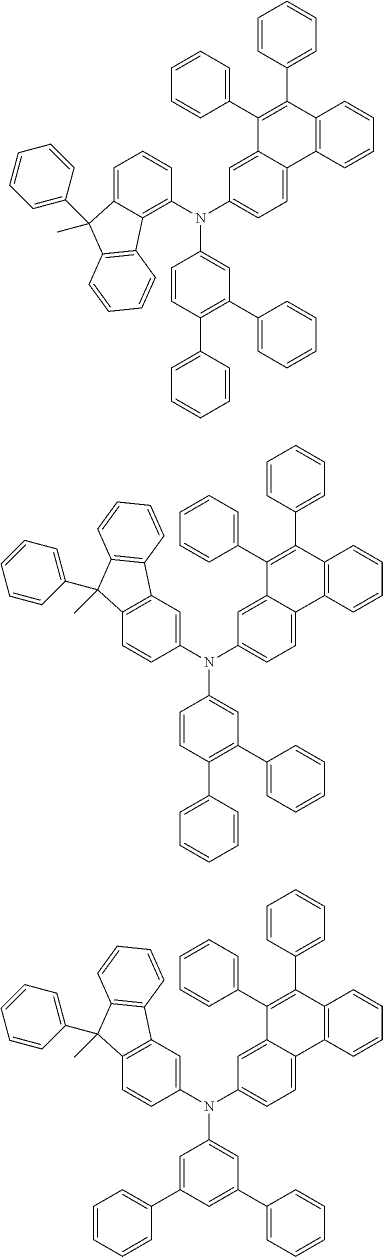 [SMILES: CC1(c2ccccc2)c2ccccc2-c2c(N(c3ccc(-c4ccccc4)c(-c4ccccc4)c3)c3ccc4c(c3)c(-c3ccccc3)c(-c3ccccc3)c3ccccc34)cccc21 ; CC1(c2ccccc2)c2ccccc2-c2cc(N(c3ccc(-c4ccccc4)c(-c4ccccc4)c3)c3ccc4c(c3)c(-c3ccccc3)c(-c3ccccc3)c3ccccc34)ccc21 ; CC1(c2ccccc2)c2ccccc2-c2cc(N(c3cc(-c4ccccc4)cc(-c4ccccc4)c3)c3ccc4c(c3)c(-c3ccccc3)c(-c3ccccc3)c3ccccc34)ccc21]

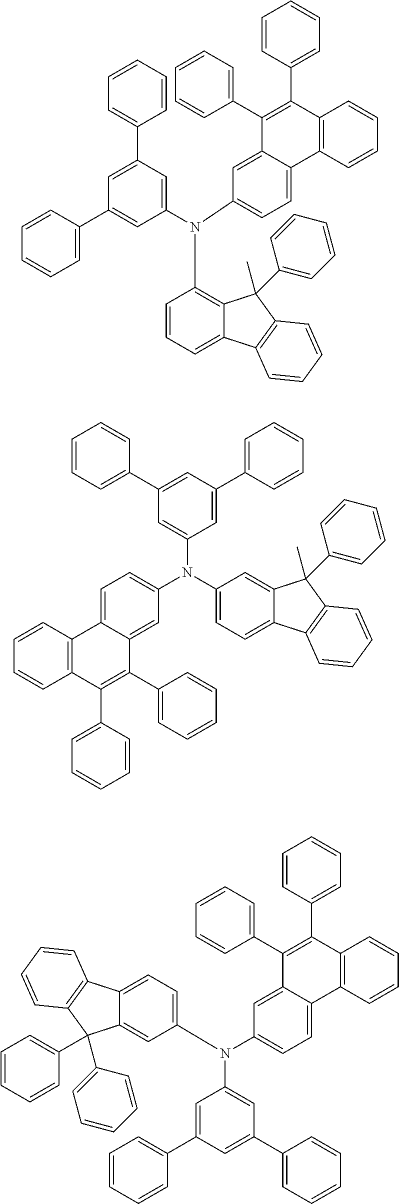

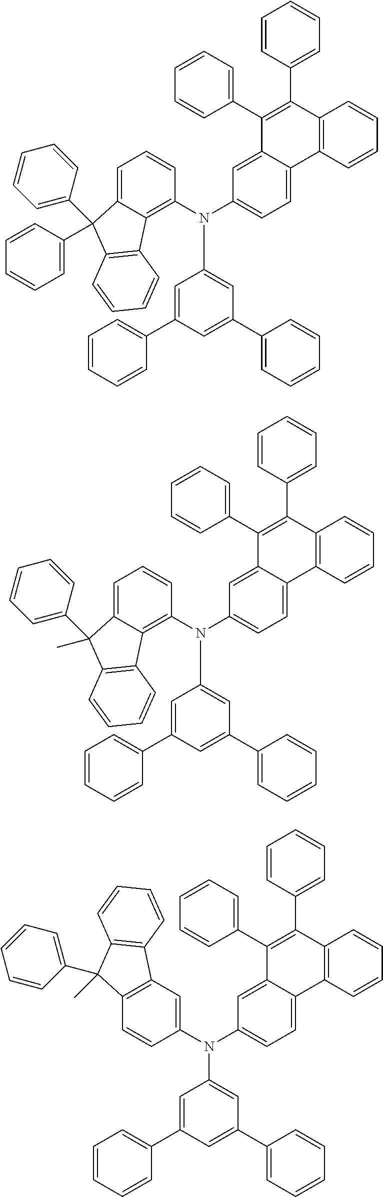

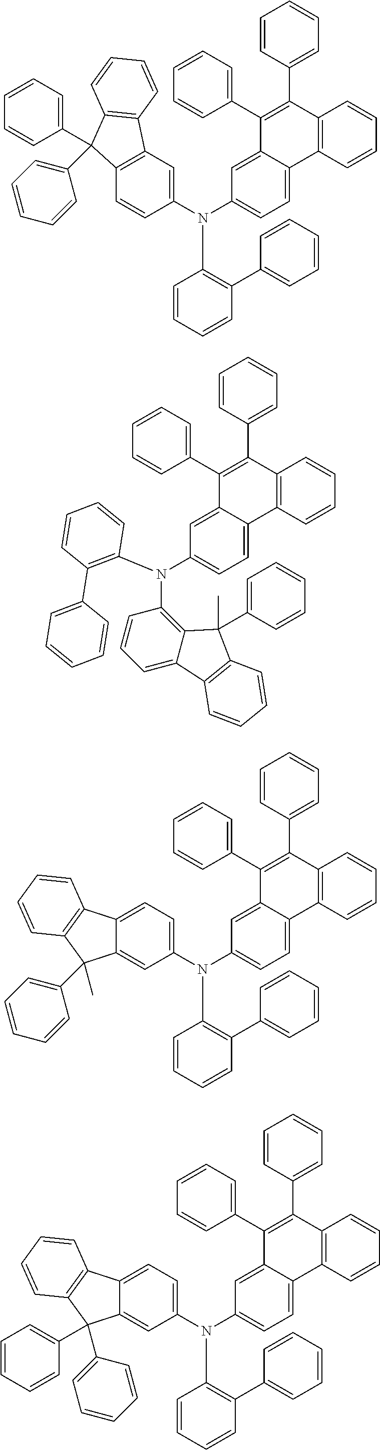

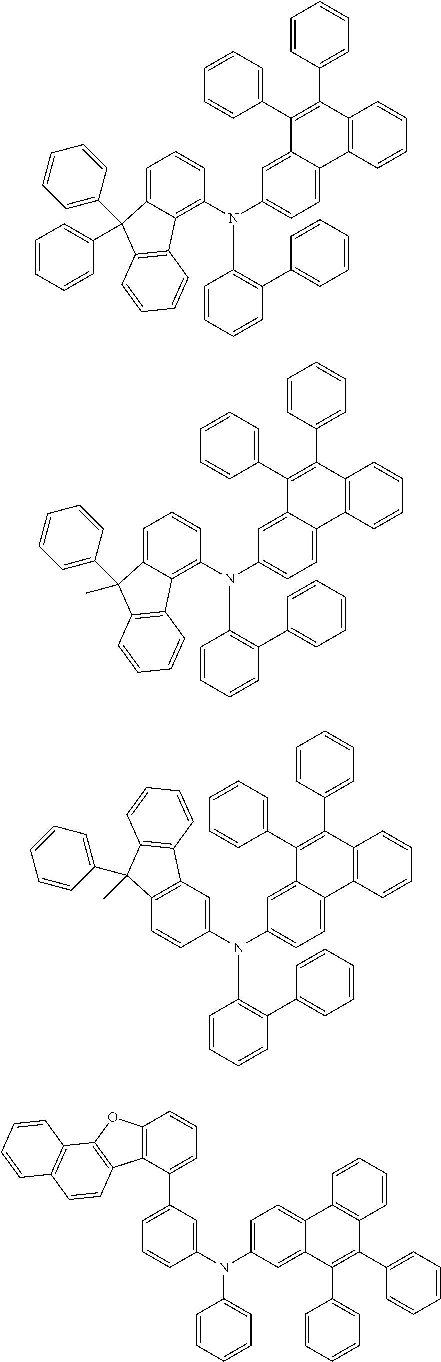 [SMILES: c1ccc(-c2ccccc2N(c2ccc3c(c2)c(-c2ccccc2)c(-c2ccccc2)c2ccccc23)c2cccc3c2-c2ccccc2C3(c2ccccc2)c2ccccc2)cc1 ; CC1(c2ccccc2)c2ccccc2-c2c(N(c3ccc4c(c3)c(-c3ccccc3)c(-c3ccccc3)c3ccccc34)c3ccccc3-c3ccccc3)cccc21 ; CC1(c2ccccc2)c2ccccc2-c2cc(N(c3ccc4c(c3)c(-c3ccccc3)c(-c3ccccc3)c3ccccc34)c3ccccc3-c3ccccc3)ccc21 ; c1ccc(-c2c(-c3ccccc3)c3cc(N(c4ccccc4)c4cccc(-c5cccc6oc7c8ccccc8ccc7c56)c4)ccc3c3ccccc23)cc1]

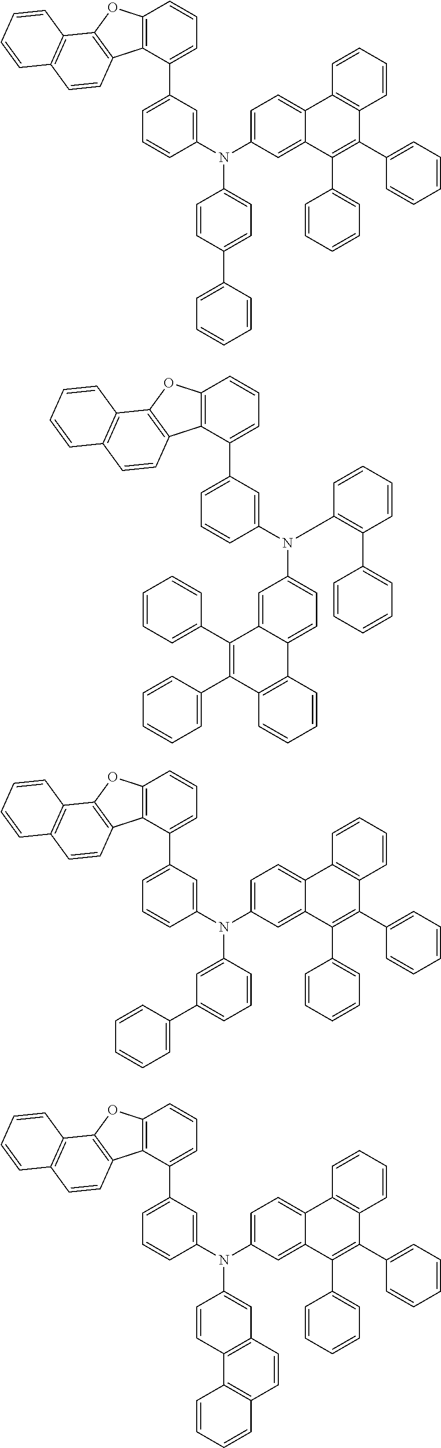

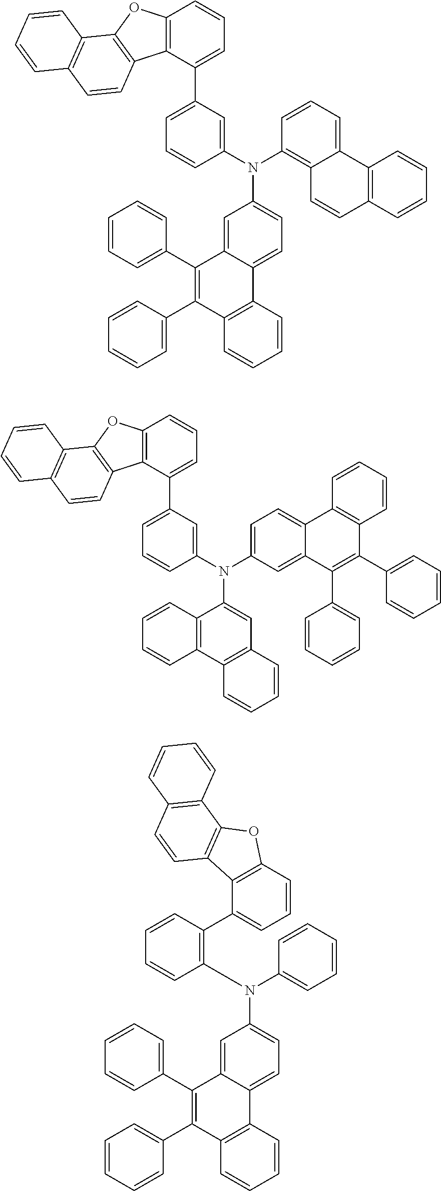 [SMILES: c1ccc(-c2c(-c3ccccc3)c3cc(N(c4cccc(-c5cccc6oc7c8ccccc8ccc7c56)c4)c4cccc5c4ccc4ccccc45)ccc3c3ccccc23)cc1 ; c1ccc(-c2c(-c3ccccc3)c3cc(N(c4cccc(-c5cccc6oc7c8ccccc8ccc7c56)c4)c4cc5ccccc5c5ccccc45)ccc3c3ccccc23)cc1 ; c1ccc(-c2c(-c3ccccc3)c3cc(N(c4ccccc4)c4ccccc4-c4cccc5oc6c7ccccc7ccc6c45)ccc3c3ccccc23)cc1]

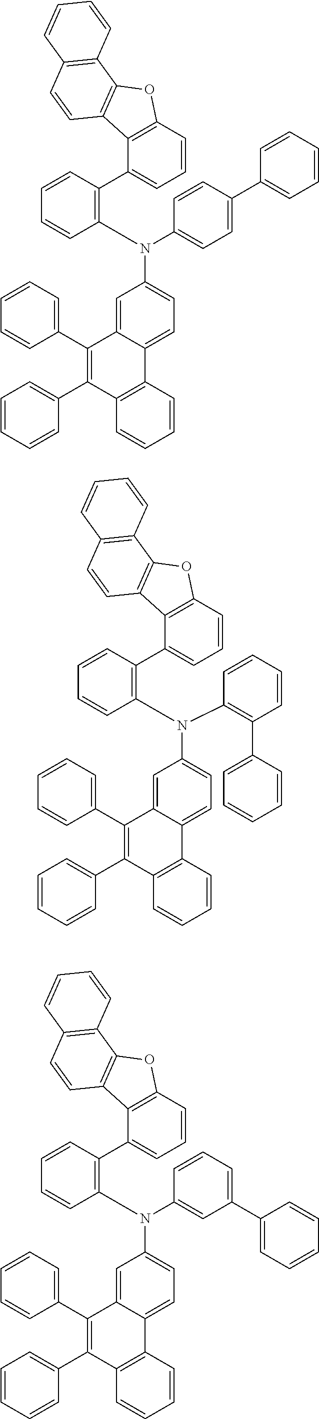

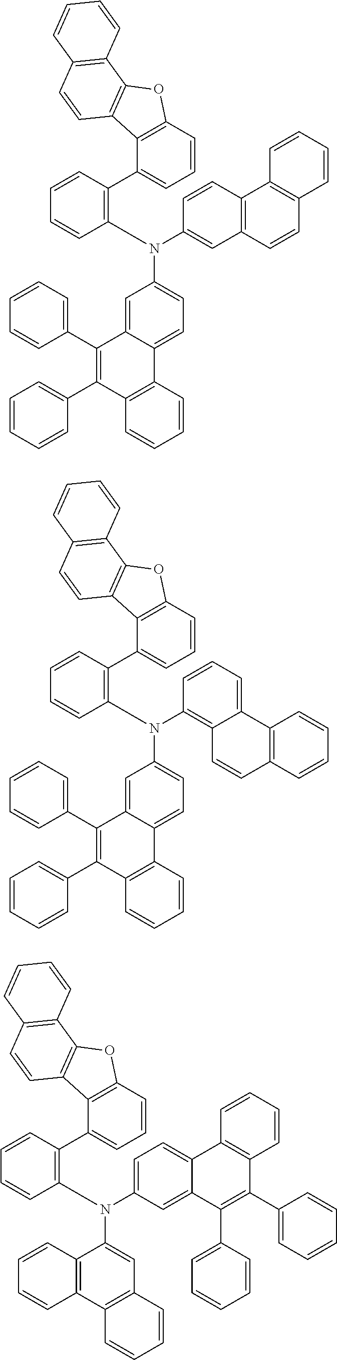

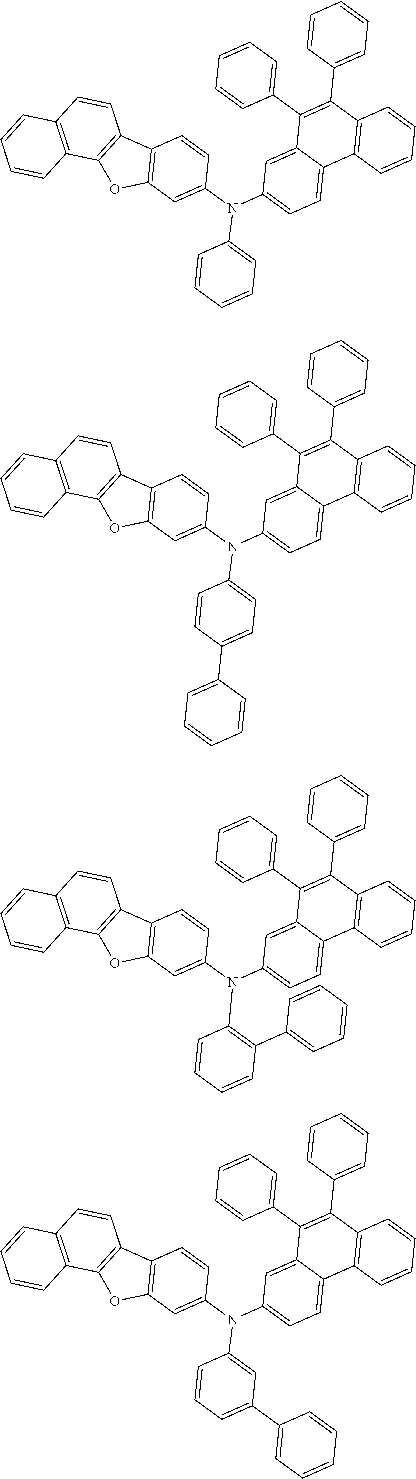 [SMILES: c1ccc(-c2c(-c3ccccc3)c3cc(N(c4ccccc4)c4ccc5c(c4)oc4c6ccccc6ccc54)ccc3c3ccccc23)cc1 ; c1ccc(-c2ccc(N(c3ccc4c(c3)oc3c5ccccc5ccc43)c3ccc4c(c3)c(-c3ccccc3)c(-c3ccccc3)c3ccccc34)cc2)cc1 ; c1ccc(-c2ccccc2N(c2ccc3c(c2)oc2c4ccccc4ccc32)c2ccc3c(c2)c(-c2ccccc2)c(-c2ccccc2)c2ccccc23)cc1 ; c1ccc(-c2cccc(N(c3ccc4c(c3)oc3c5ccccc5ccc43)c3ccc4c(c3)c(-c3ccccc3)c(-c3ccccc3)c3ccccc34)c2)cc1]

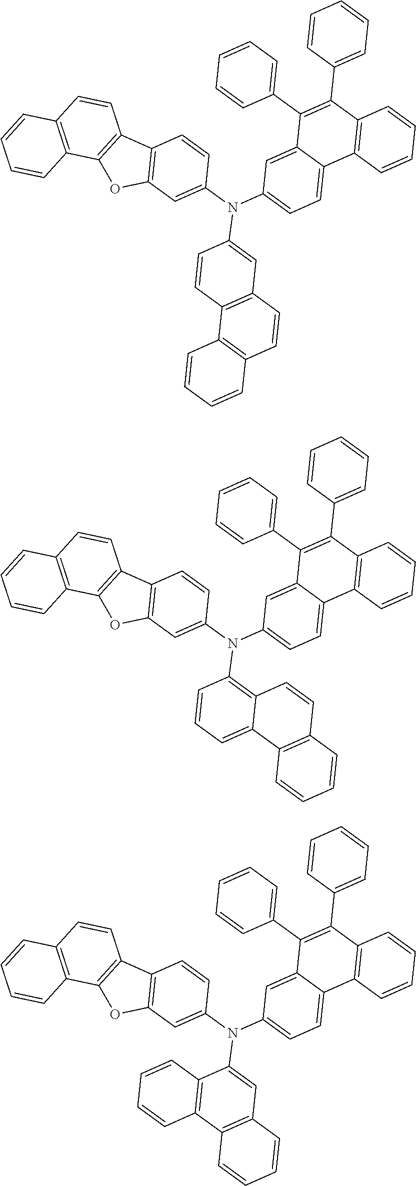

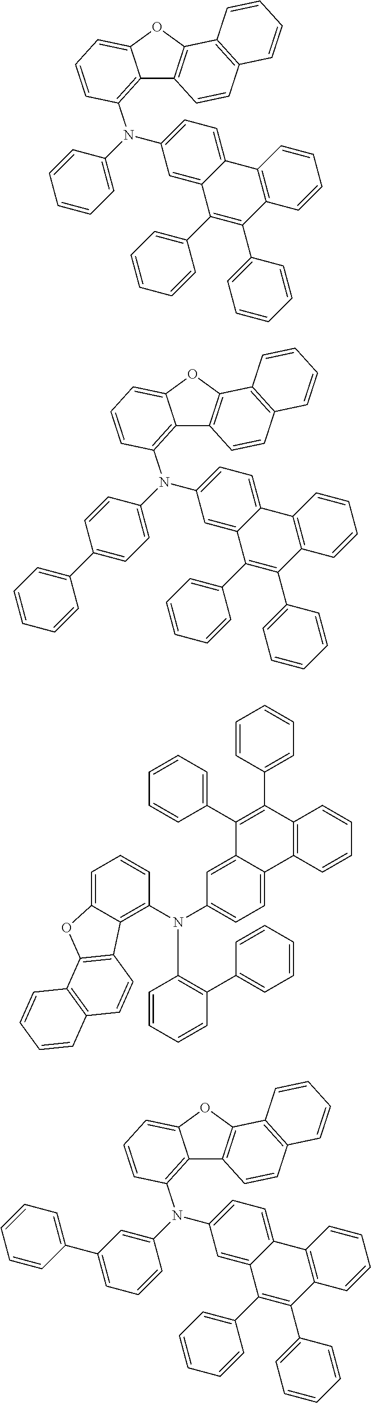

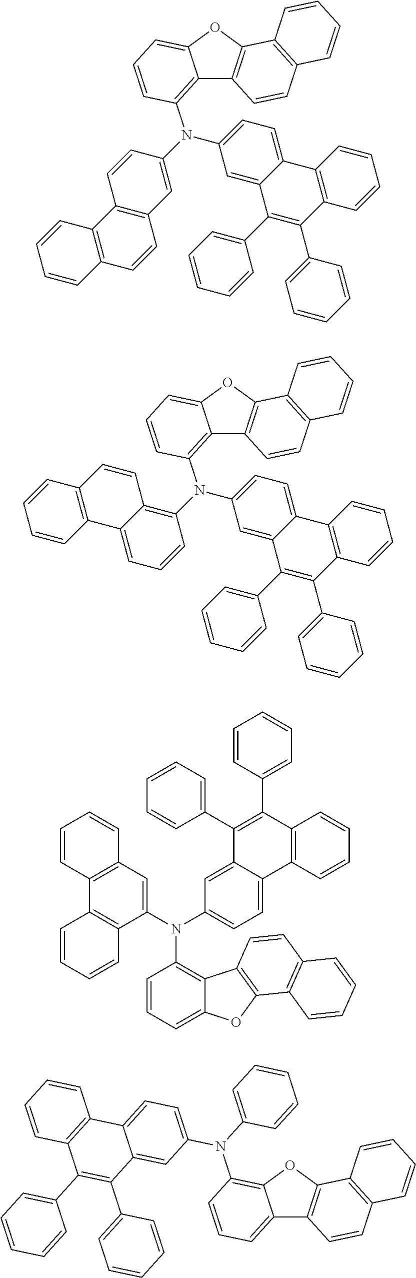

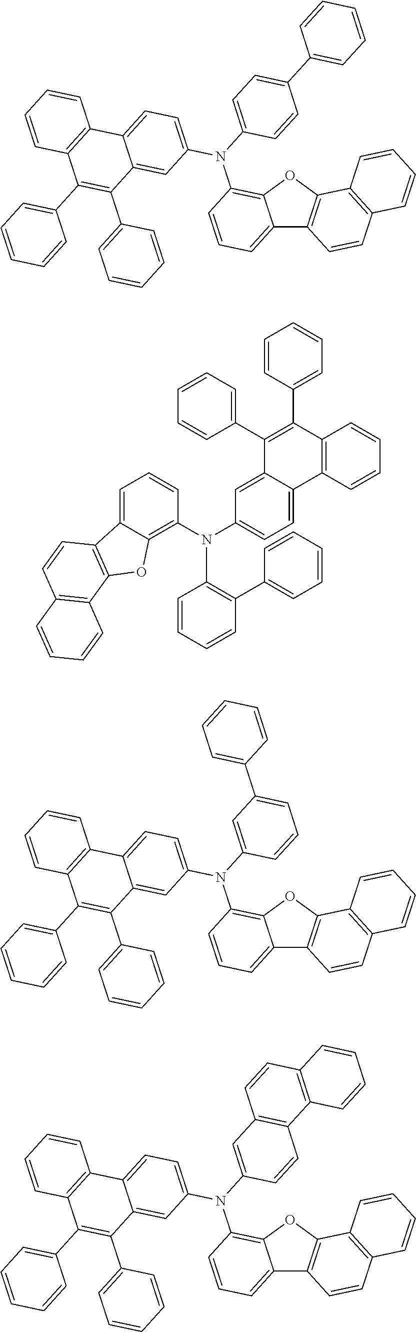 [SMILES: c1ccc(-c2ccc(N(c3ccc4c(c3)c(-c3ccccc3)c(-c3ccccc3)c3ccccc34)c3cccc4c3oc3c5ccccc5ccc43)cc2)cc1 ; c1ccc(-c2ccccc2N(c2ccc3c(c2)c(-c2ccccc2)c(-c2ccccc2)c2ccccc23)c2cccc3c2oc2c4ccccc4ccc32)cc1 ; c1ccc(-c2cccc(N(c3ccc4c(c3)c(-c3ccccc3)c(-c3ccccc3)c3ccccc34)c3cccc4c3oc3c5ccccc5ccc43)c2)cc1 ; c1ccc(-c2c(-c3ccccc3)c3cc(N(c4ccc5c(ccc6ccccc65)c4)c4cccc5c4oc4c6ccccc6ccc54)ccc3c3ccccc23)cc1]

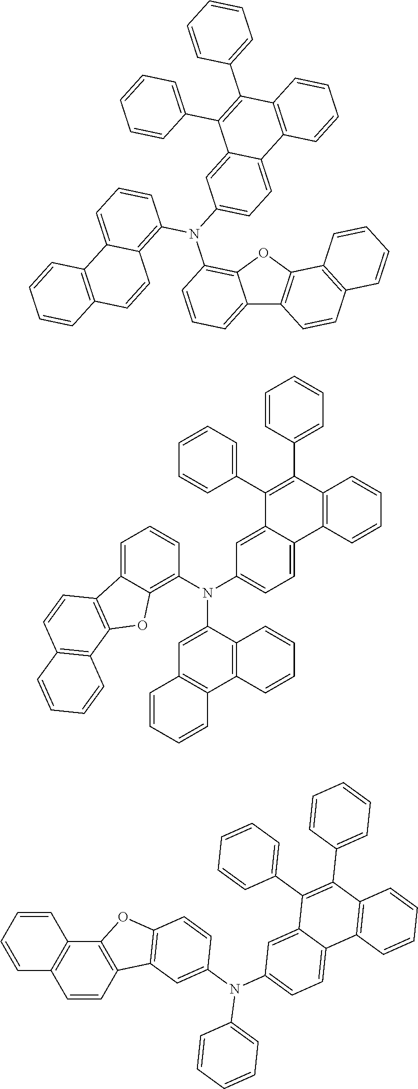 [SMILES: c1ccc(-c2c(-c3ccccc3)c3cc(N(c4cccc5c4ccc4ccccc45)c4cccc5c4oc4c6ccccc6ccc54)ccc3c3ccccc23)cc1 ; c1ccc(-c2c(-c3ccccc3)c3cc(N(c4cc5ccccc5c5ccccc45)c4cccc5c4oc4c6ccccc6ccc54)ccc3c3ccccc23)cc1 ; c1ccc(-c2c(-c3ccccc3)c3cc(N(c4ccccc4)c4ccc5oc6c7ccccc7ccc6c5c4)ccc3c3ccccc23)cc1]

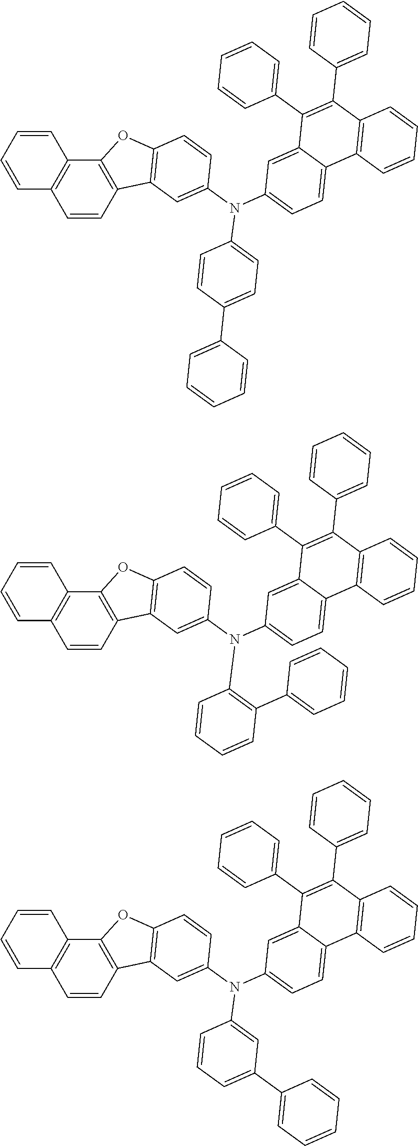 [SMILES: c1ccc(-c2ccc(N(c3ccc4c(c3)c(-c3ccccc3)c(-c3ccccc3)c3ccccc34)c3ccc4oc5c6ccccc6ccc5c4c3)cc2)cc1 ; c1ccc(-c2ccccc2N(c2ccc3c(c2)c(-c2ccccc2)c(-c2ccccc2)c2ccccc23)c2ccc3oc4c5ccccc5ccc4c3c2)cc1 ; c1ccc(-c2cccc(N(c3ccc4c(c3)c(-c3ccccc3)c(-c3ccccc3)c3ccccc34)c3ccc4oc5c6ccccc6ccc5c4c3)c2)cc1]

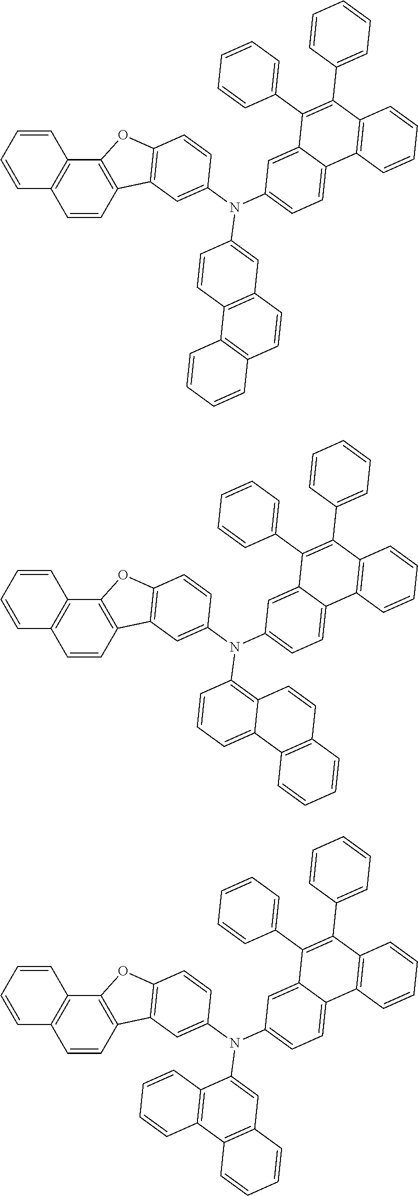

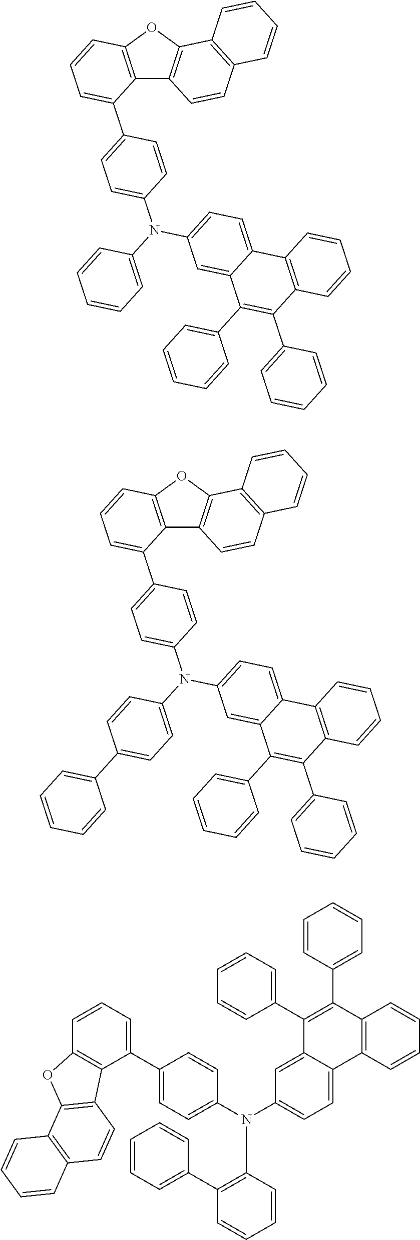

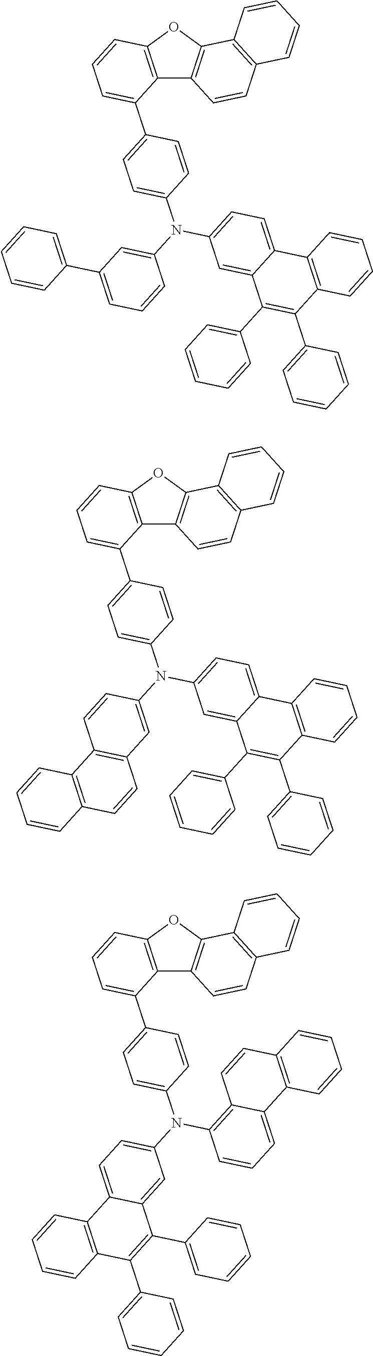

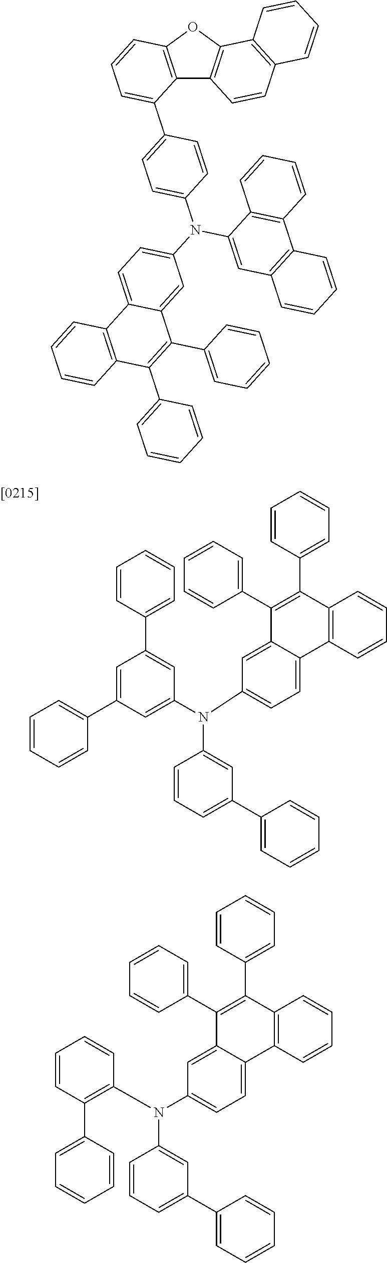 [SMILES: c1ccc(-c2c(-c3ccccc3)c3cc(N(c4ccc(-c5cccc6oc7c8ccccc8ccc7c56)cc4)c4cc5ccccc5c5ccccc45)ccc3c3ccccc23)cc1 ; c1ccc(-c2cccc(N(c3cc(-c4ccccc4)cc(-c4ccccc4)c3)c3ccc4c(c3)c(-c3ccccc3)c(-c3ccccc3)c3ccccc34)c2)cc1 ; c1ccc(-c2cccc(N(c3ccc4c(c3)c(-c3ccccc3)c(-c3ccccc3)c3ccccc34)c3ccccc3-c3ccccc3)c2)cc1]

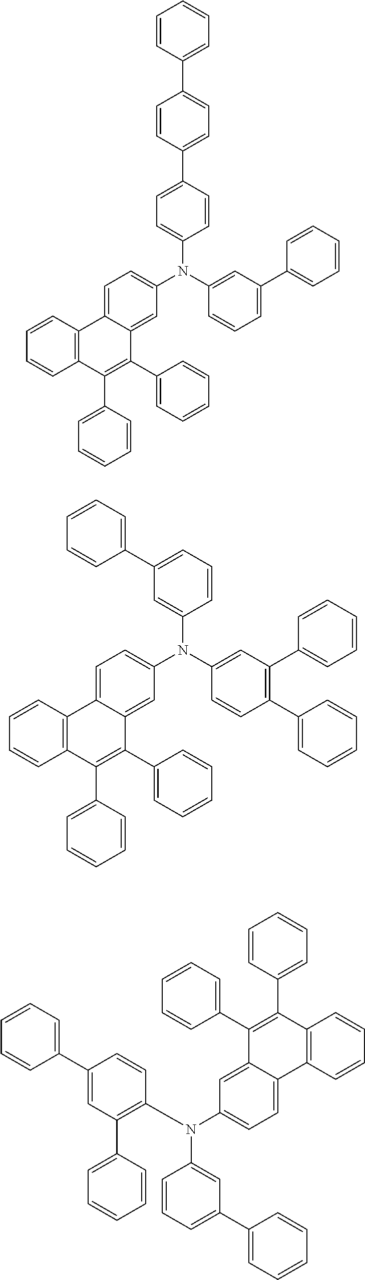

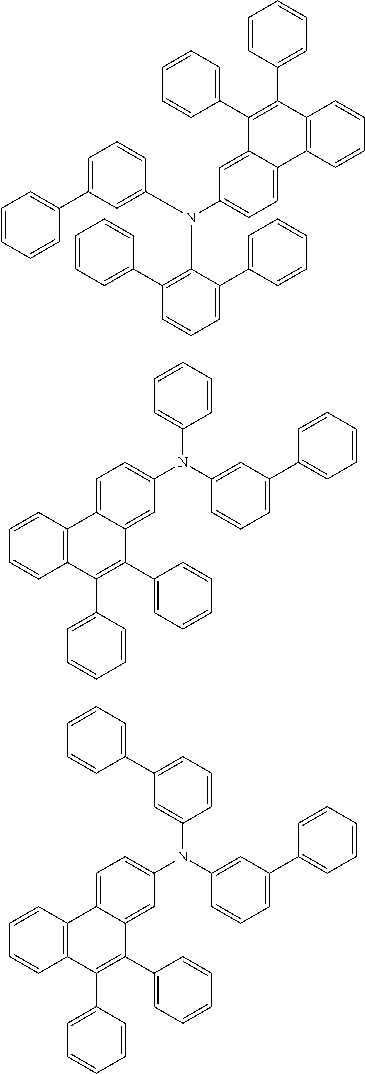

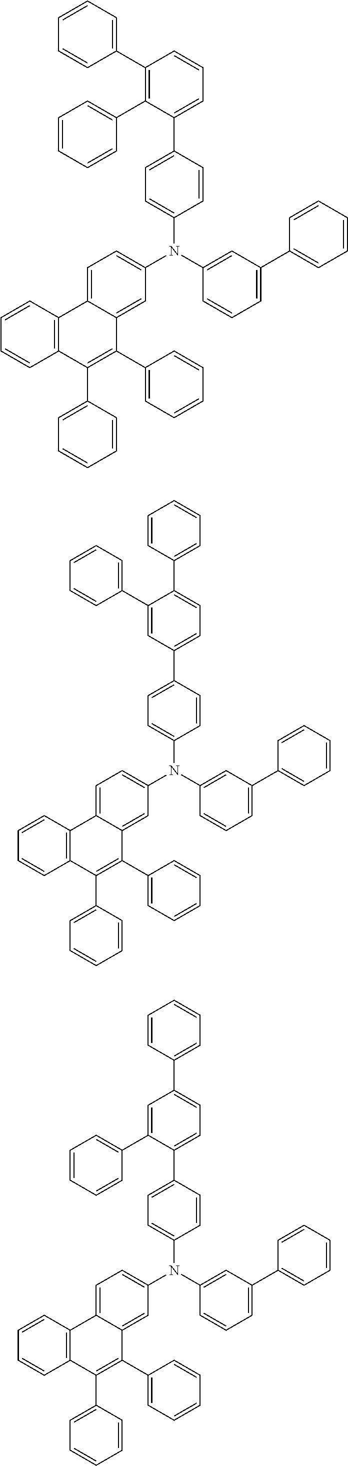 [SMILES: c1ccc(-c2cccc(N(c3ccc(-c4cccc(-c5ccccc5)c4-c4ccccc4)cc3)c3ccc4c(c3)c(-c3ccccc3)c(-c3ccccc3)c3ccccc34)c2)cc1 ; c1ccc(-c2cccc(N(c3ccc(-c4ccc(-c5ccccc5)c(-c5ccccc5)c4)cc3)c3ccc4c(c3)c(-c3ccccc3)c(-c3ccccc3)c3ccccc34)c2)cc1 ; c1ccc(-c2cccc(N(c3ccc(-c4ccc(-c5ccccc5)cc4-c4ccccc4)cc3)c3ccc4c(c3)c(-c3ccccc3)c(-c3ccccc3)c3ccccc34)c2)cc1]

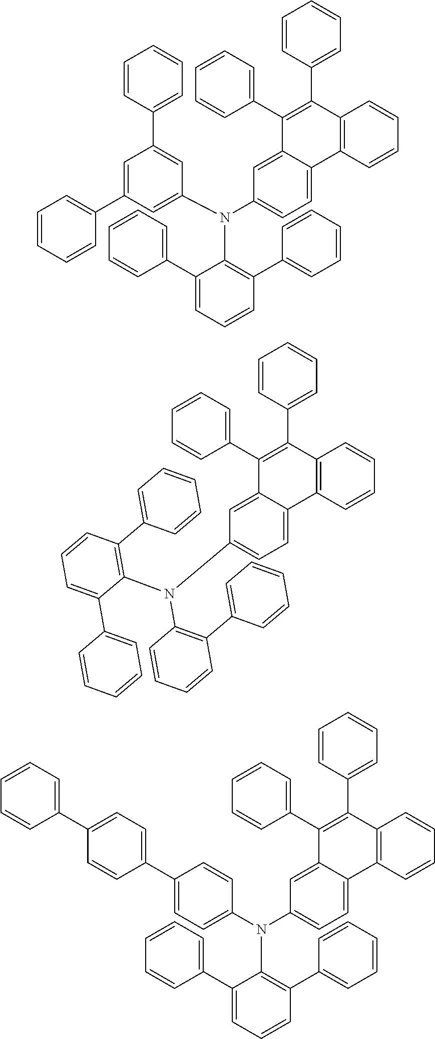 [SMILES: c1ccc(-c2cc(-c3ccccc3)cc(N(c3ccc4c(c3)c(-c3ccccc3)c(-c3ccccc3)c3ccccc34)c3c(-c4ccccc4)cccc3-c3ccccc3)c2)cc1 ; c1ccc(-c2ccccc2N(c2ccc3c(c2)c(-c2ccccc2)c(-c2ccccc2)c2ccccc23)c2c(-c3ccccc3)cccc2-c2ccccc2)cc1 ; c1ccc(-c2ccc(-c3ccc(N(c4ccc5c(c4)c(-c4ccccc4)c(-c4ccccc4)c4ccccc45)c4c(-c5ccccc5)cccc4-c4ccccc4)cc3)cc2)cc1]

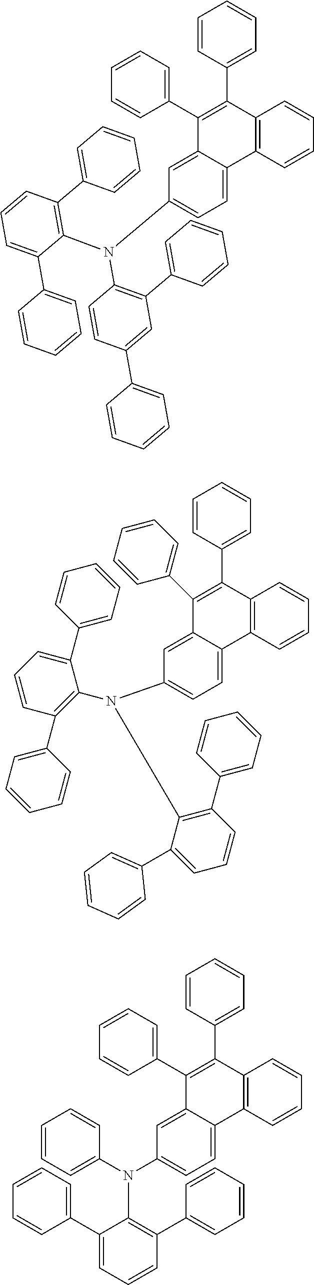 [SMILES: c1ccc(-c2ccc(N(c3ccc4c(c3)c(-c3ccccc3)c(-c3ccccc3)c3ccccc34)c3c(-c4ccccc4)cccc3-c3ccccc3)c(-c3ccccc3)c2)cc1 ; c1ccc(-c2cccc(-c3ccccc3)c2N(c2ccc3c(c2)c(-c2ccccc2)c(-c2ccccc2)c2ccccc23)c2c(-c3ccccc3)cccc2-c2ccccc2)cc1 ; c1ccc(-c2cccc(-c3ccccc3)c2N(c2ccccc2)c2ccc3c(c2)c(-c2ccccc2)c(-c2ccccc2)c2ccccc23)cc1]

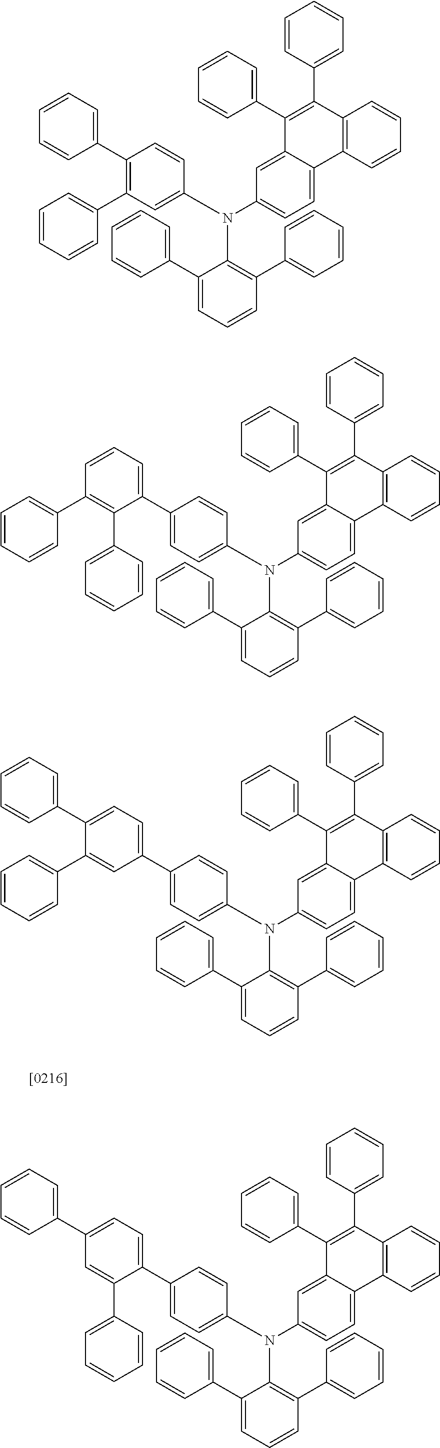 [SMILES: c1ccc(-c2ccc(N(c3ccc4c(c3)c(-c3ccccc3)c(-c3ccccc3)c3ccccc34)c3c(-c4ccccc4)cccc3-c3ccccc3)cc2-c2ccccc2)cc1 ; c1ccc(-c2cccc(-c3ccc(N(c4ccc5c(c4)c(-c4ccccc4)c(-c4ccccc4)c4ccccc45)c4c(-c5ccccc5)cccc4-c4ccccc4)cc3)c2-c2ccccc2)cc1 ; c1ccc(-c2ccc(-c3ccc(N(c4ccc5c(c4)c(-c4ccccc4)c(-c4ccccc4)c4ccccc45)c4c(-c5ccccc5)cccc4-c4ccccc4)cc3)cc2-c2ccccc2)cc1 ; c1ccc(-c2ccc(-c3ccc(N(c4ccc5c(c4)c(-c4ccccc4)c(-c4ccccc4)c4ccccc45)c4c(-c5ccccc5)cccc4-c4ccccc4)cc3)c(-c3ccccc3)c2)cc1]

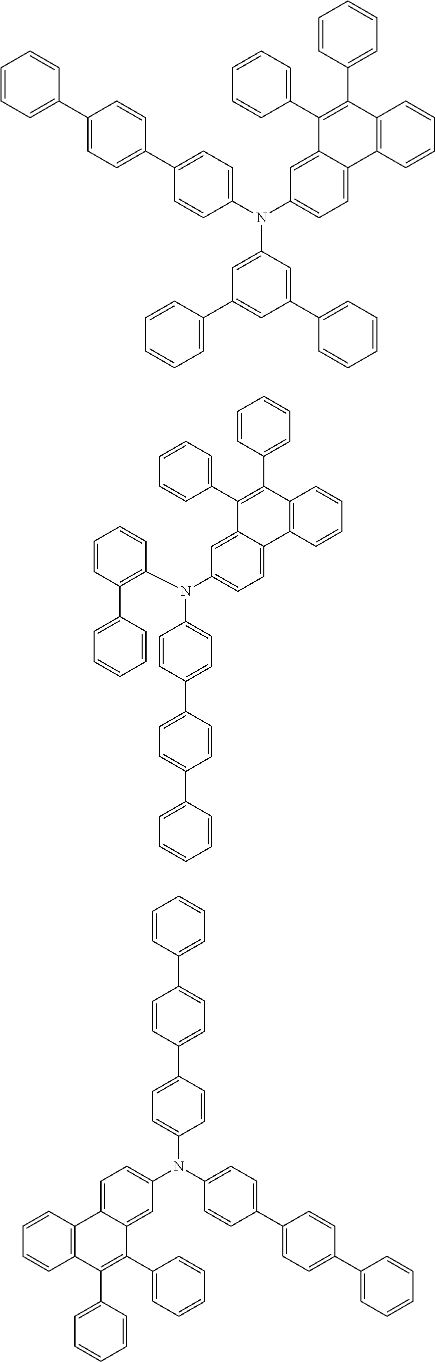

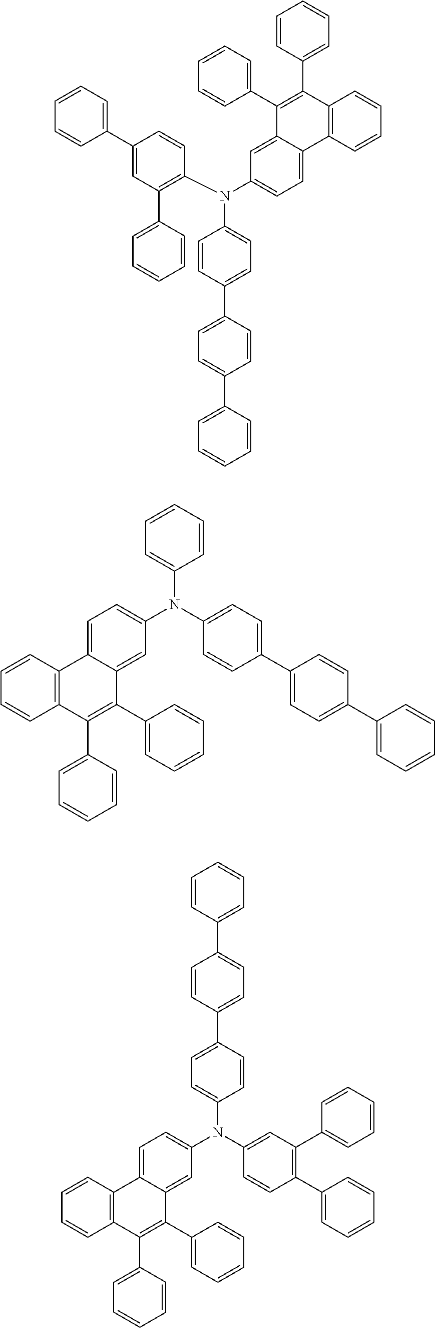 [SMILES: c1ccc(-c2ccc(-c3ccc(N(c4ccc5c(c4)c(-c4ccccc4)c(-c4ccccc4)c4ccccc45)c4ccc(-c5ccccc5)cc4-c4ccccc4)cc3)cc2)cc1 ; c1ccc(-c2ccc(-c3ccc(N(c4ccccc4)c4ccc5c(c4)c(-c4ccccc4)c(-c4ccccc4)c4ccccc45)cc3)cc2)cc1 ; c1ccc(-c2ccc(-c3ccc(N(c4ccc(-c5ccccc5)c(-c5ccccc5)c4)c4ccc5c(c4)c(-c4ccccc4)c(-c4ccccc4)c4ccccc45)cc3)cc2)cc1]

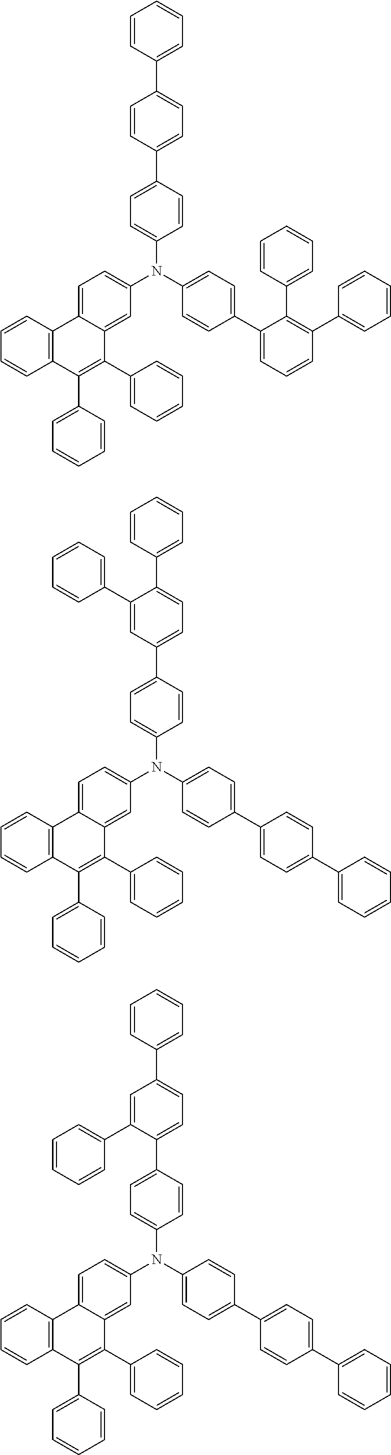 [SMILES: c1ccc(-c2ccc(-c3ccc(N(c4ccc(-c5cccc(-c6ccccc6)c5-c5ccccc5)cc4)c4ccc5c(c4)c(-c4ccccc4)c(-c4ccccc4)c4ccccc45)cc3)cc2)cc1 ; c1ccc(-c2ccc(-c3ccc(N(c4ccc(-c5ccc(-c6ccccc6)c(-c6ccccc6)c5)cc4)c4ccc5c(c4)c(-c4ccccc4)c(-c4ccccc4)c4ccccc45)cc3)cc2)cc1 ; c1ccc(-c2ccc(-c3ccc(N(c4ccc(-c5ccc(-c6ccccc6)cc5-c5ccccc5)cc4)c4ccc5c(c4)c(-c4ccccc4)c(-c4ccccc4)c4ccccc45)cc3)cc2)cc1]

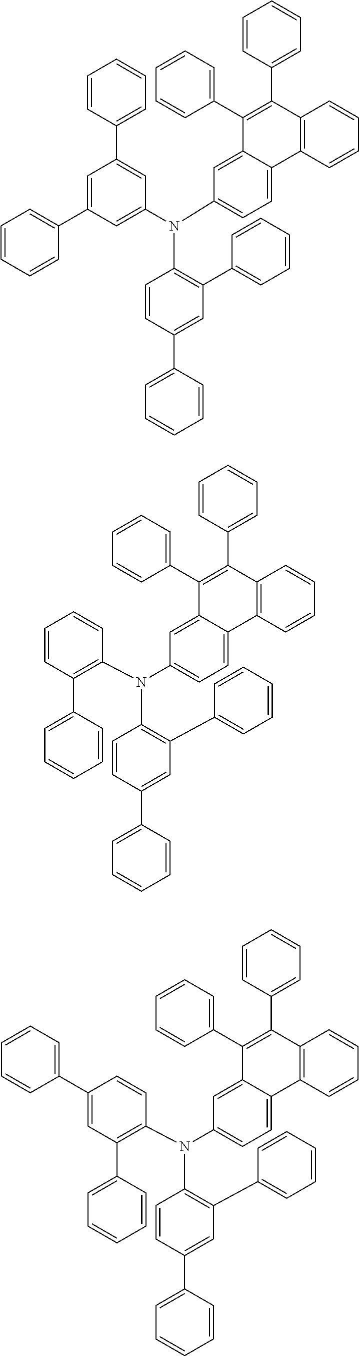 [SMILES: c1ccc(-c2cc(-c3ccccc3)cc(N(c3ccc4c(c3)c(-c3ccccc3)c(-c3ccccc3)c3ccccc34)c3ccc(-c4ccccc4)cc3-c3ccccc3)c2)cc1 ; c1ccc(-c2ccc(N(c3ccc4c(c3)c(-c3ccccc3)c(-c3ccccc3)c3ccccc34)c3ccccc3-c3ccccc3)c(-c3ccccc3)c2)cc1 ; c1ccc(-c2ccc(N(c3ccc4c(c3)c(-c3ccccc3)c(-c3ccccc3)c3ccccc34)c3ccc(-c4ccccc4)cc3-c3ccccc3)c(-c3ccccc3)c2)cc1]

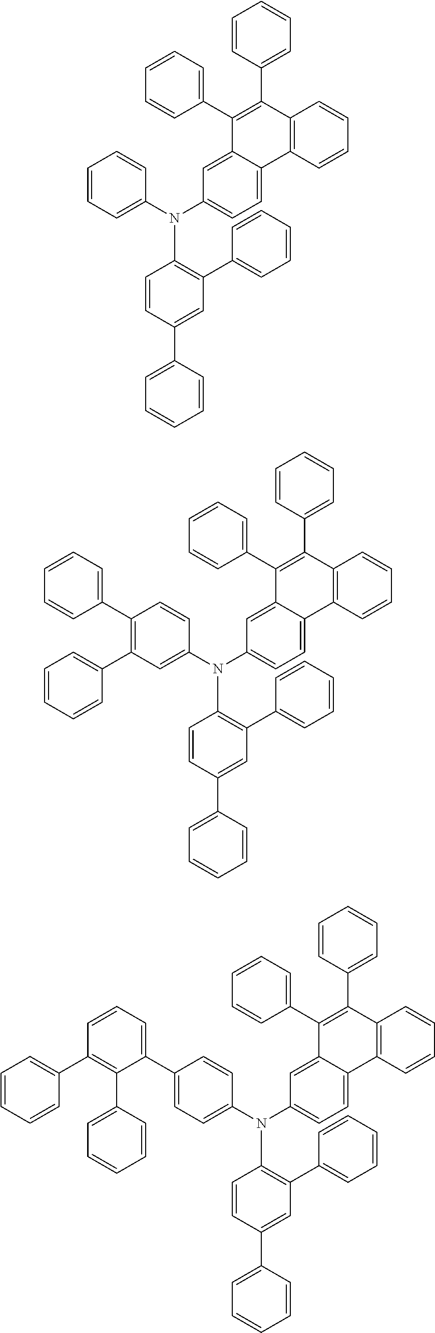 [SMILES: c1ccc(-c2ccc(N(c3ccccc3)c3ccc4c(c3)c(-c3ccccc3)c(-c3ccccc3)c3ccccc34)c(-c3ccccc3)c2)cc1 ; c1ccc(-c2ccc(N(c3ccc(-c4ccccc4)c(-c4ccccc4)c3)c3ccc4c(c3)c(-c3ccccc3)c(-c3ccccc3)c3ccccc34)c(-c3ccccc3)c2)cc1 ; c1ccc(-c2ccc(N(c3ccc(-c4cccc(-c5ccccc5)c4-c4ccccc4)cc3)c3ccc4c(c3)c(-c3ccccc3)c(-c3ccccc3)c3ccccc34)c(-c3ccccc3)c2)cc1]

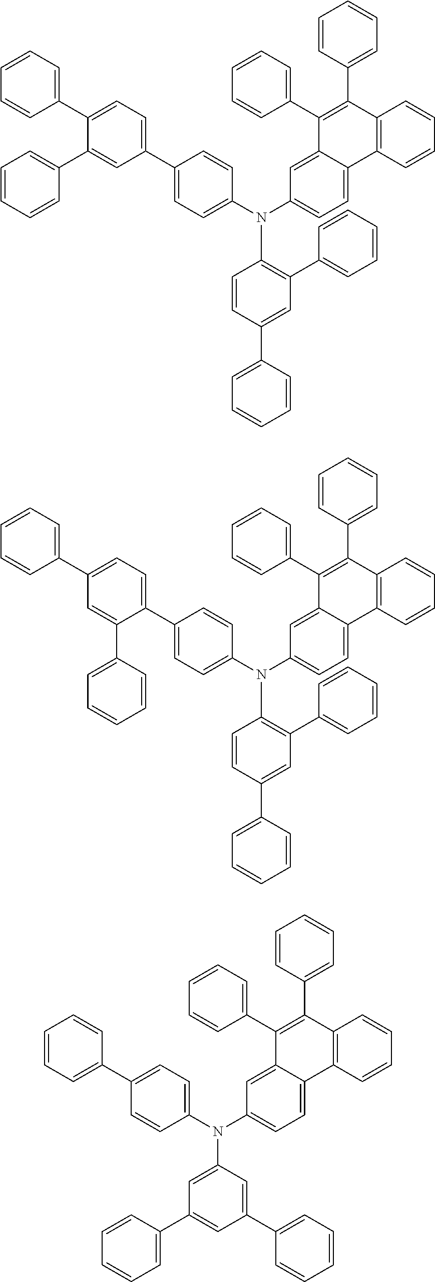 [SMILES: c1ccc(-c2ccc(N(c3ccc(-c4ccc(-c5ccccc5)c(-c5ccccc5)c4)cc3)c3ccc4c(c3)c(-c3ccccc3)c(-c3ccccc3)c3ccccc34)c(-c3ccccc3)c2)cc1 ; c1ccc(-c2ccc(-c3ccc(N(c4ccc5c(c4)c(-c4ccccc4)c(-c4ccccc4)c4ccccc45)c4ccc(-c5ccccc5)cc4-c4ccccc4)cc3)c(-c3ccccc3)c2)cc1 ; c1ccc(-c2ccc(N(c3cc(-c4ccccc4)cc(-c4ccccc4)c3)c3ccc4c(c3)c(-c3ccccc3)c(-c3ccccc3)c3ccccc34)cc2)cc1]

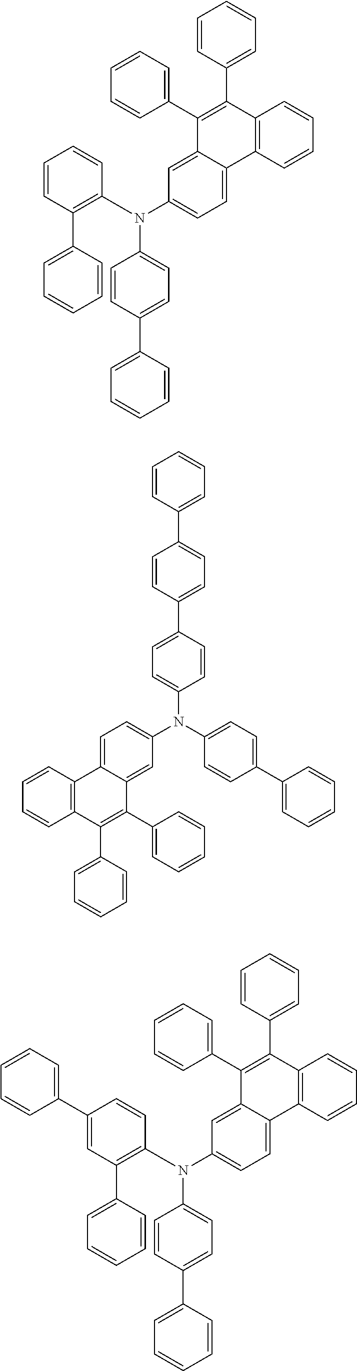 [SMILES: c1ccc(-c2ccc(N(c3ccc4c(c3)c(-c3ccccc3)c(-c3ccccc3)c3ccccc34)c3ccccc3-c3ccccc3)cc2)cc1 ; c1ccc(-c2ccc(-c3ccc(N(c4ccc(-c5ccccc5)cc4)c4ccc5c(c4)c(-c4ccccc4)c(-c4ccccc4)c4ccccc45)cc3)cc2)cc1 ; c1ccc(-c2ccc(N(c3ccc4c(c3)c(-c3ccccc3)c(-c3ccccc3)c3ccccc34)c3ccc(-c4ccccc4)cc3-c3ccccc3)cc2)cc1]

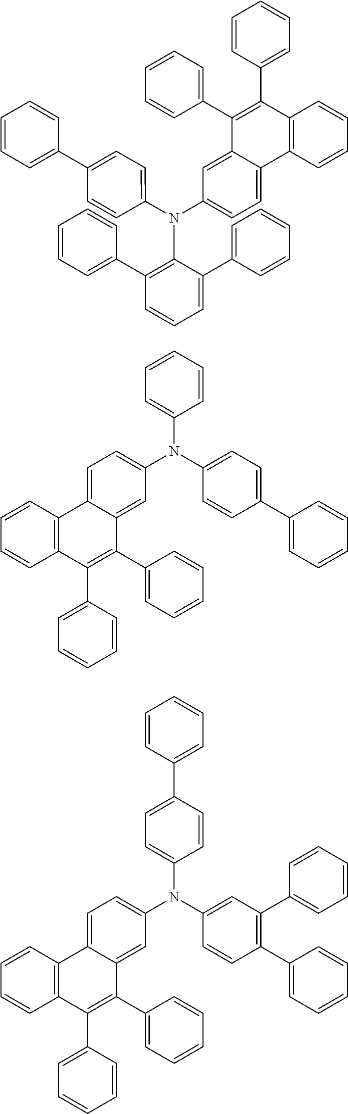

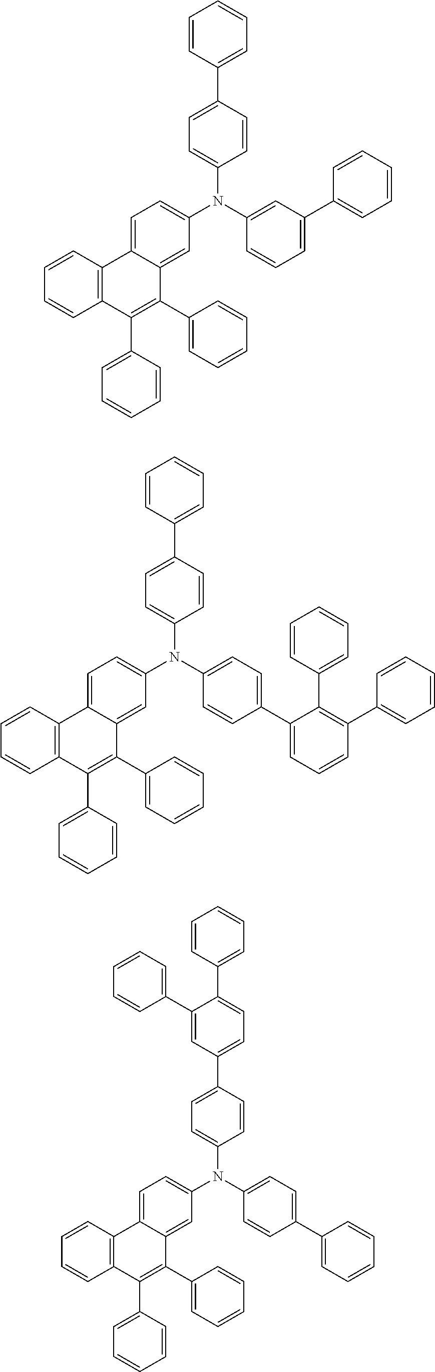 [SMILES: c1ccc(-c2ccc(N(c3cccc(-c4ccccc4)c3)c3ccc4c(c3)c(-c3ccccc3)c(-c3ccccc3)c3ccccc34)cc2)cc1 ; c1ccc(-c2ccc(N(c3ccc(-c4cccc(-c5ccccc5)c4-c4ccccc4)cc3)c3ccc4c(c3)c(-c3ccccc3)c(-c3ccccc3)c3ccccc34)cc2)cc1 ; c1ccc(-c2ccc(N(c3ccc(-c4ccc(-c5ccccc5)c(-c5ccccc5)c4)cc3)c3ccc4c(c3)c(-c3ccccc3)c(-c3ccccc3)c3ccccc34)cc2)cc1]

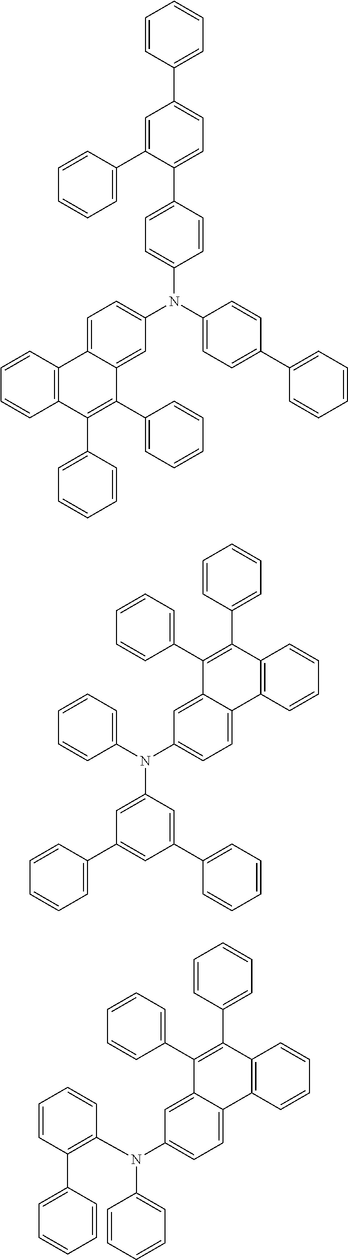

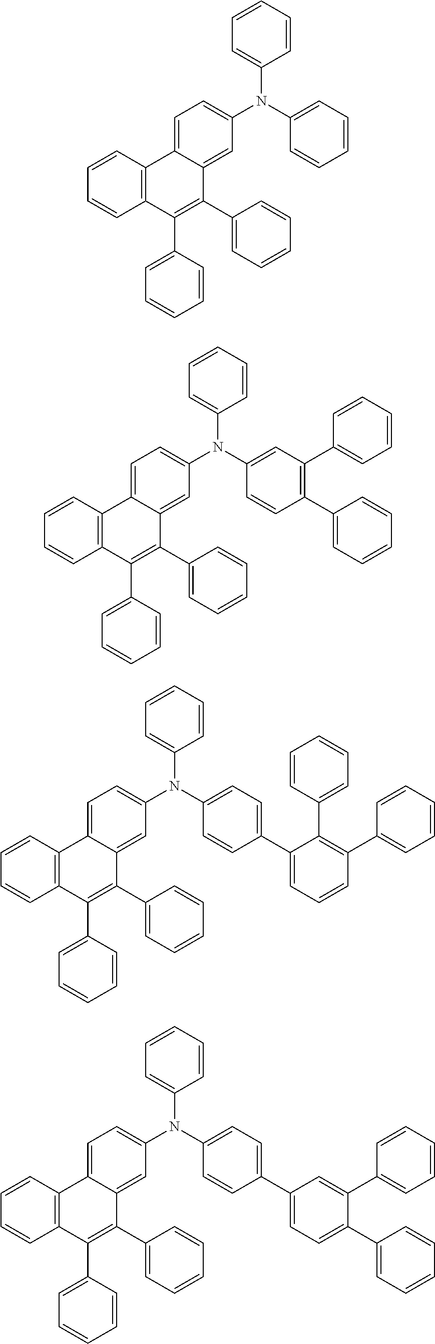

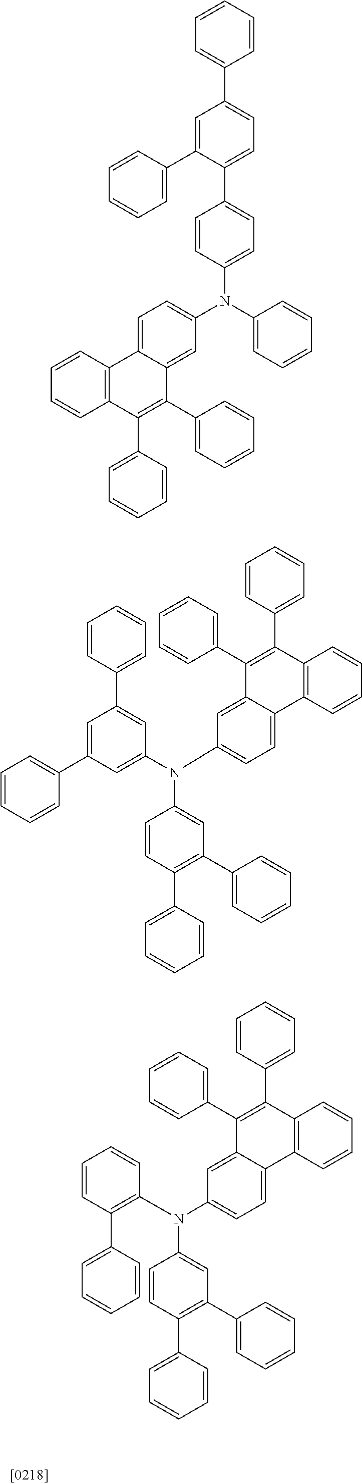 [SMILES: c1ccc(-c2ccc(-c3ccc(N(c4ccccc4)c4ccc5c(c4)c(-c4ccccc4)c(-c4ccccc4)c4ccccc45)cc3)c(-c3ccccc3)c2)cc1 ; c1ccc(-c2cc(-c3ccccc3)cc(N(c3ccc(-c4ccccc4)c(-c4ccccc4)c3)c3ccc4c(c3)c(-c3ccccc3)c(-c3ccccc3)c3ccccc34)c2)cc1 ; c1ccc(-c2ccc(N(c3ccc4c(c3)c(-c3ccccc3)c(-c3ccccc3)c3ccccc34)c3ccccc3-c3ccccc3)cc2-c2ccccc2)cc1]

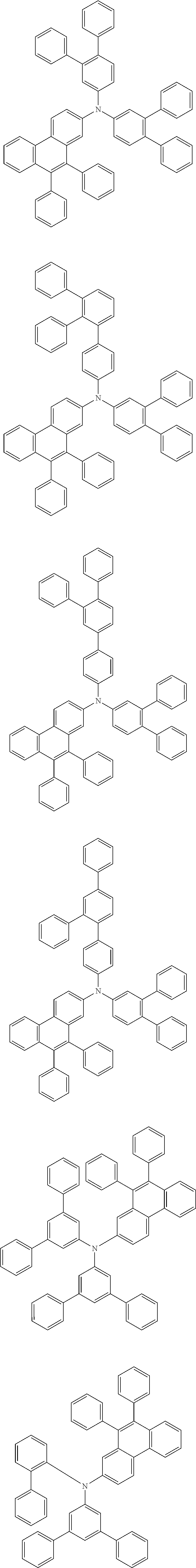

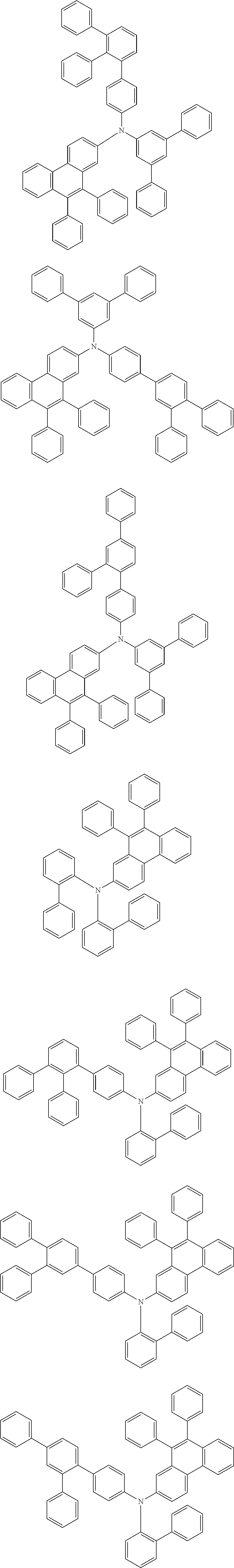

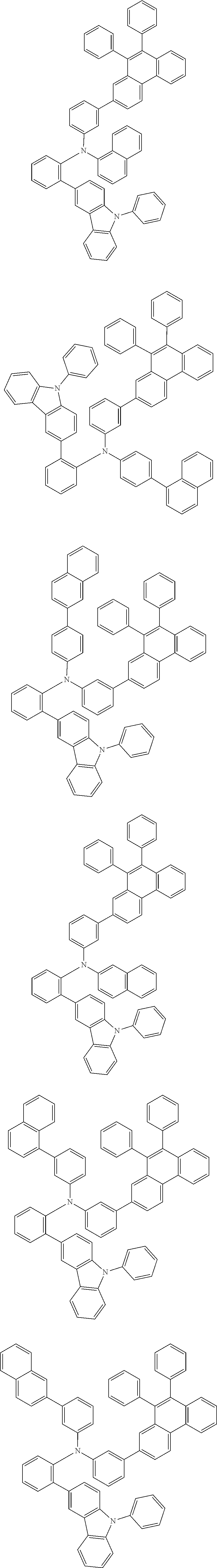

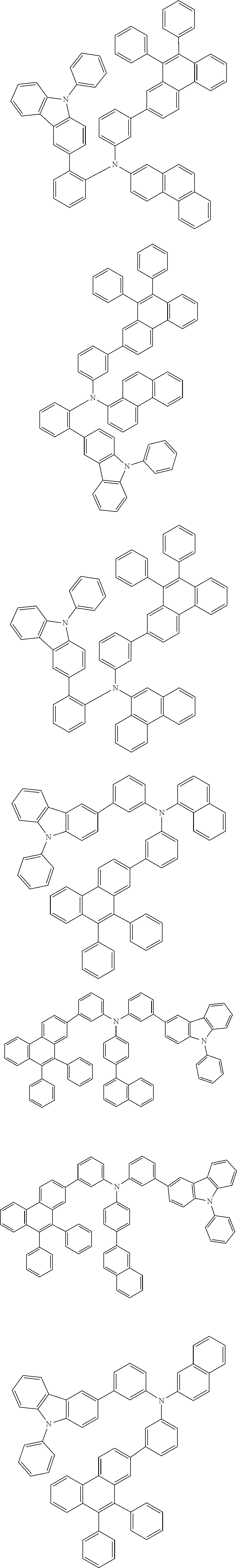

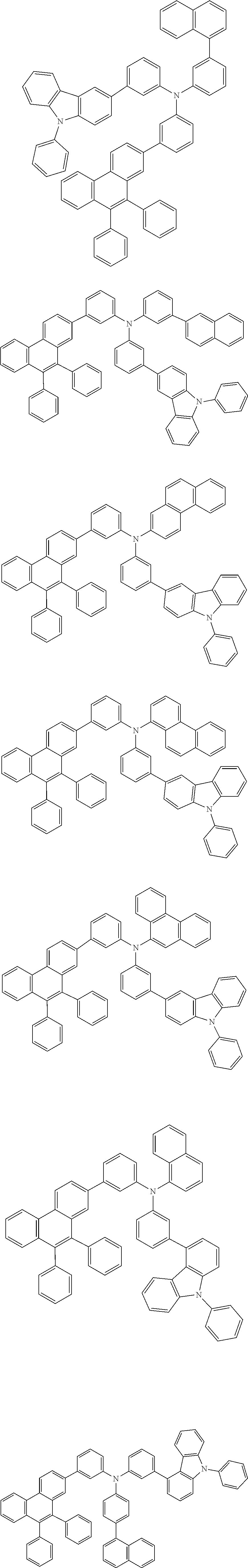

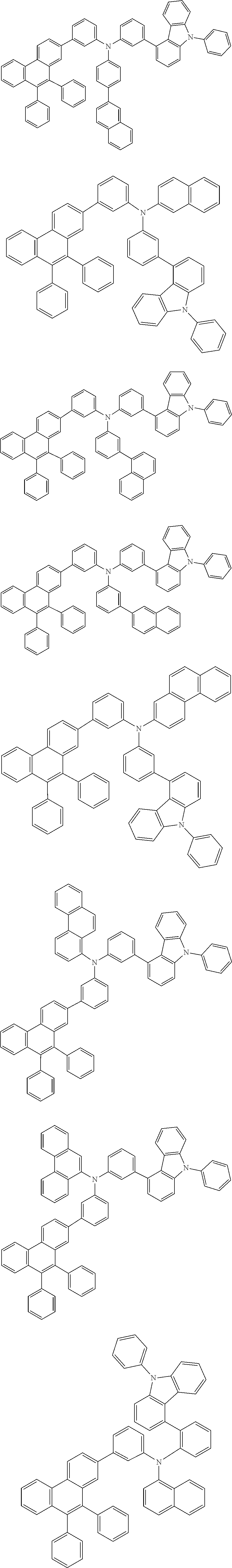

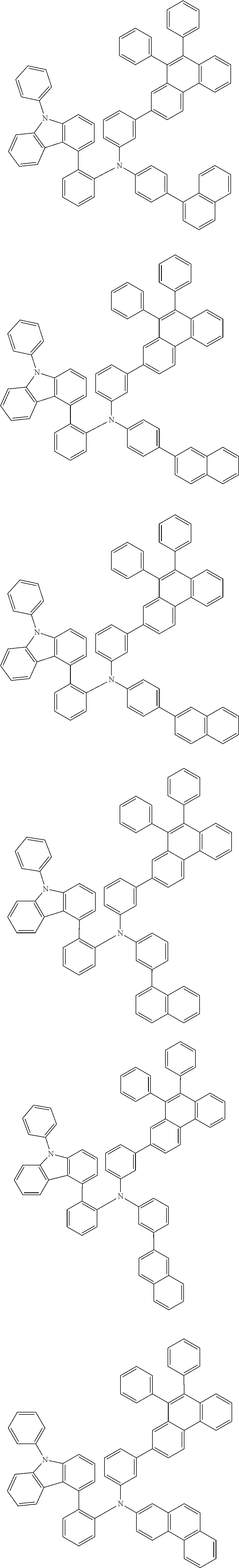

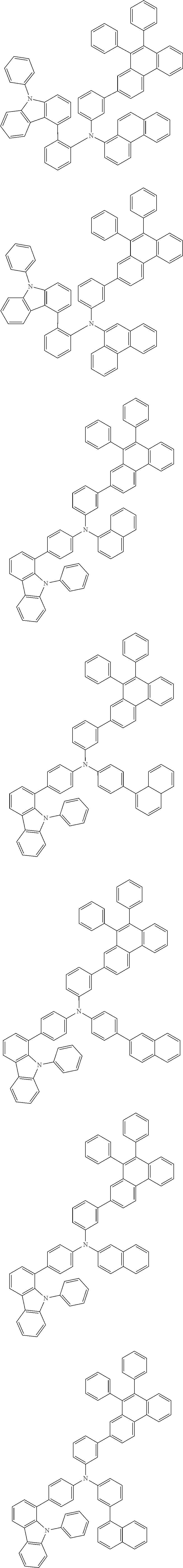

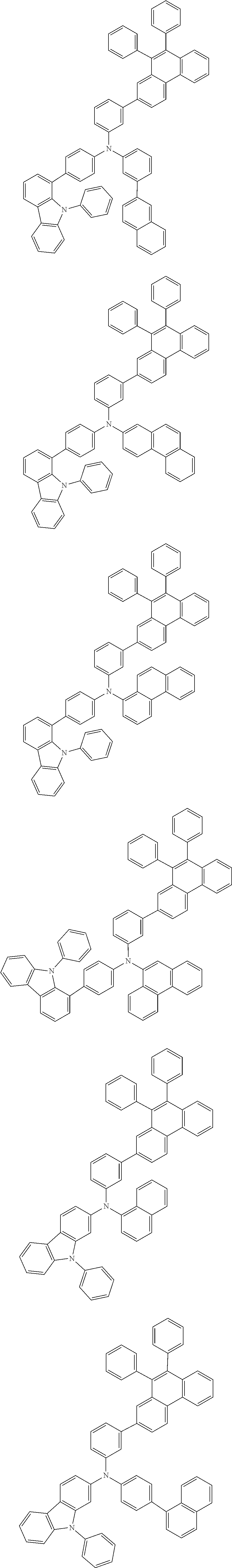

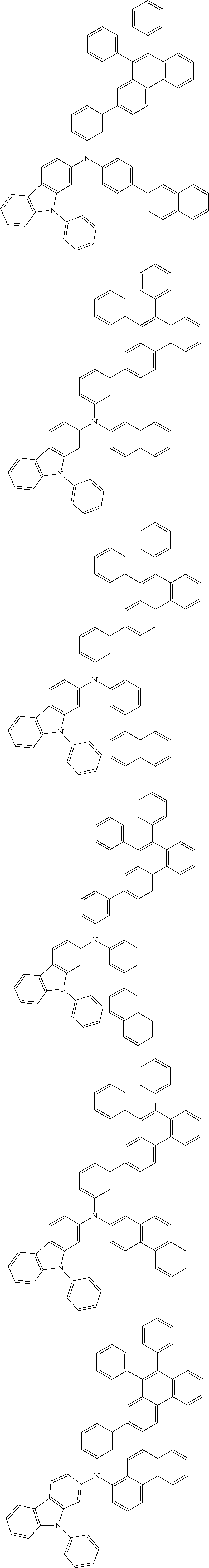

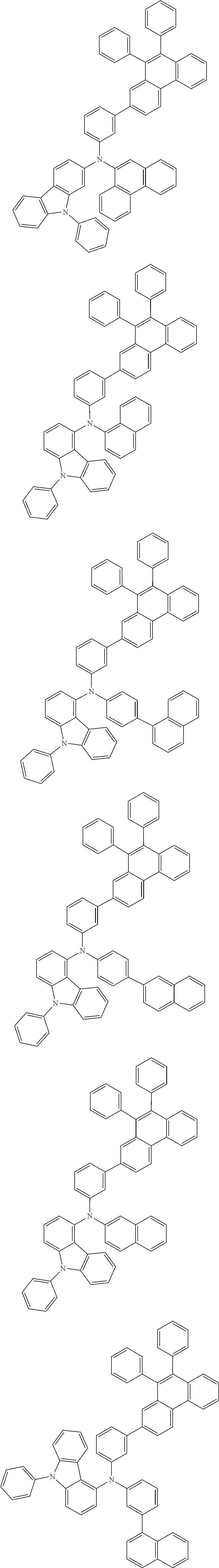

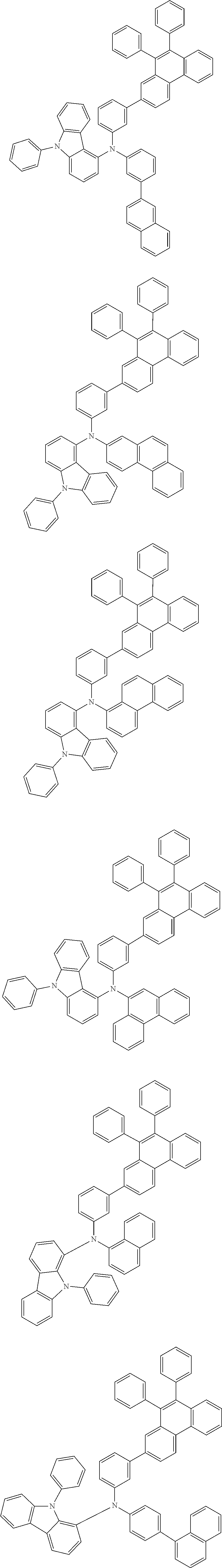

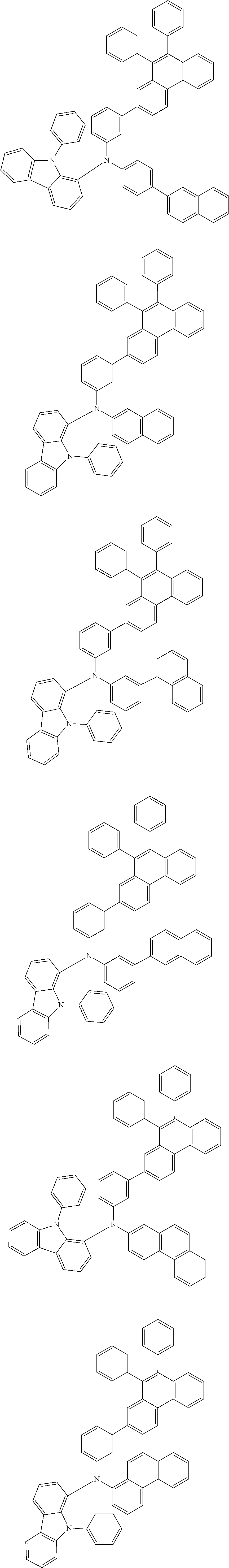

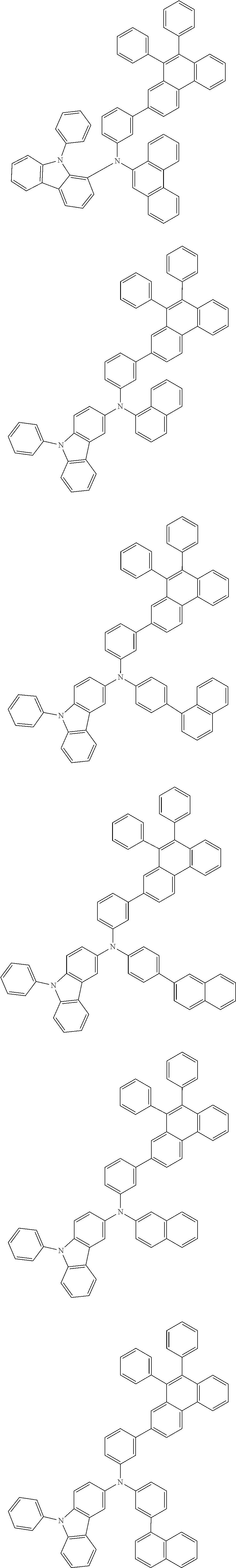

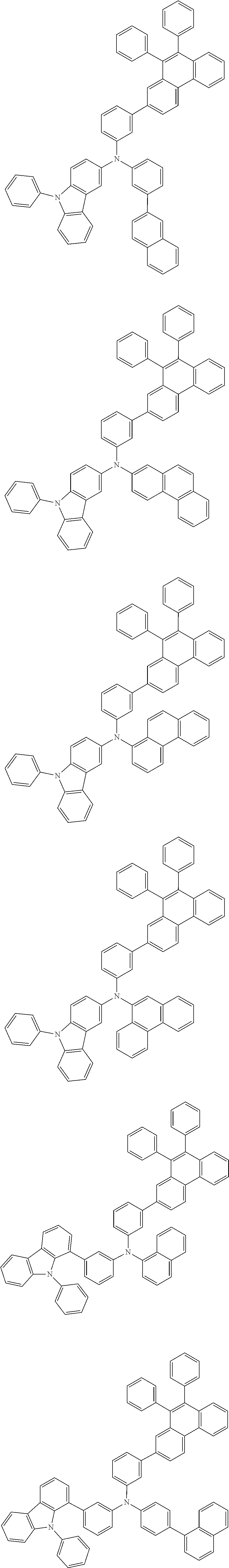

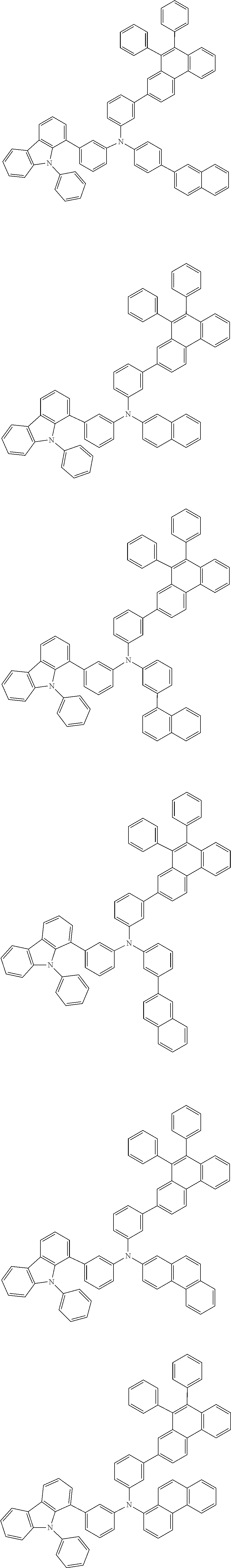

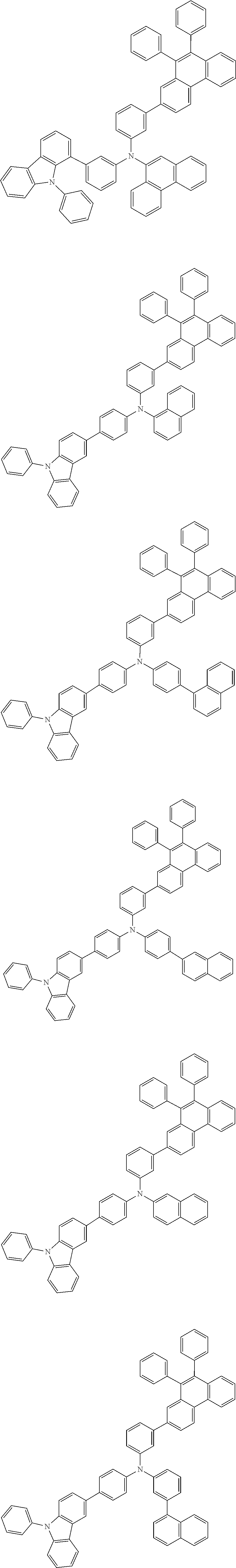

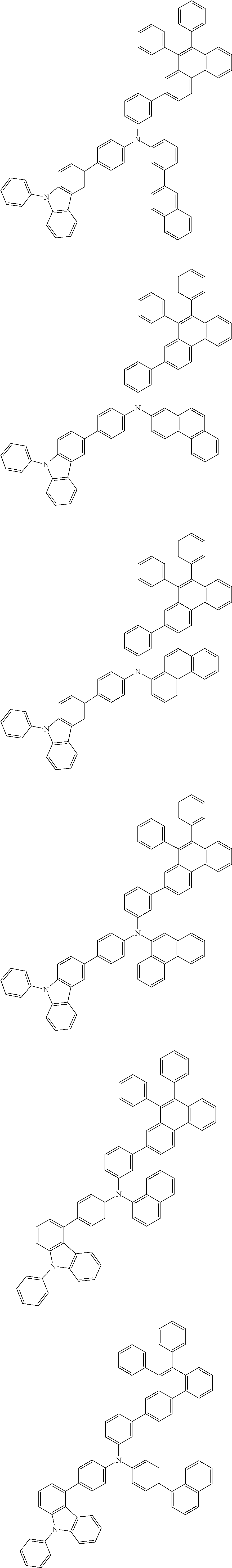

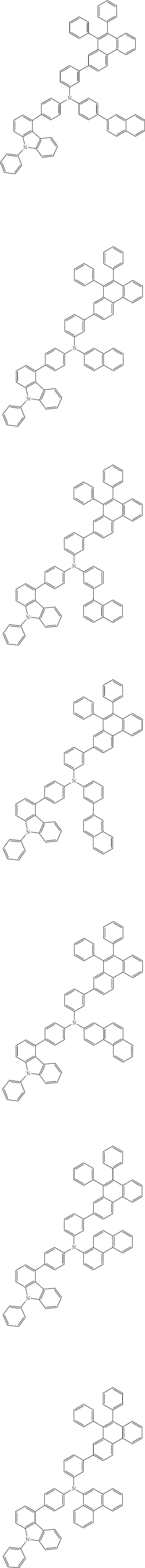

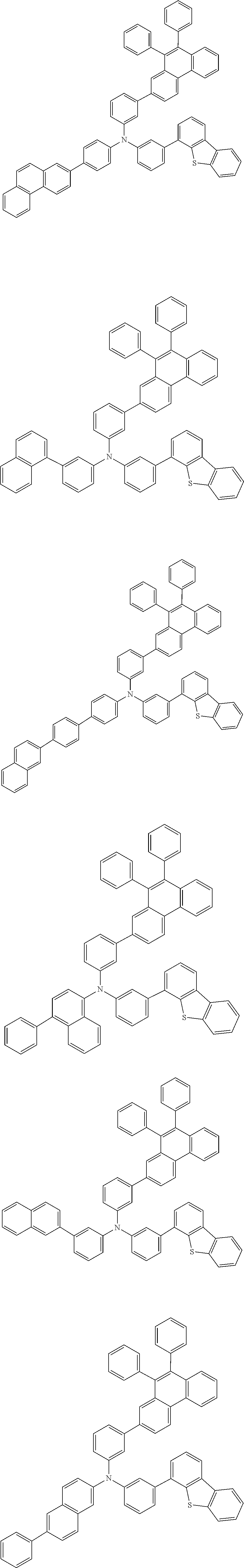

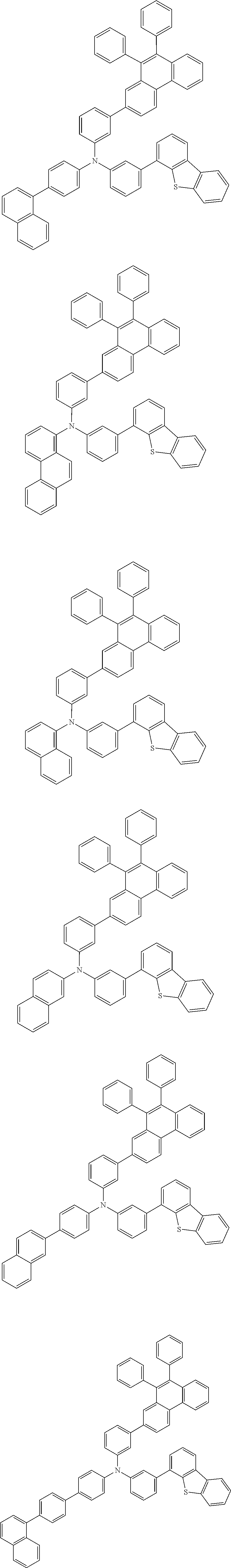

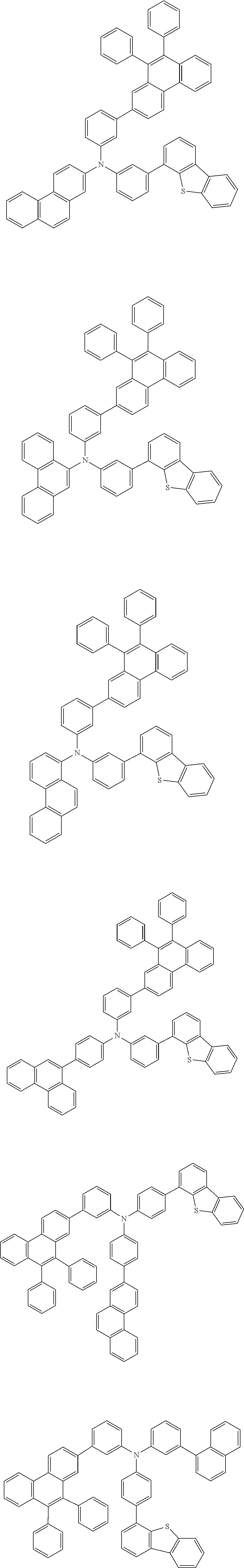

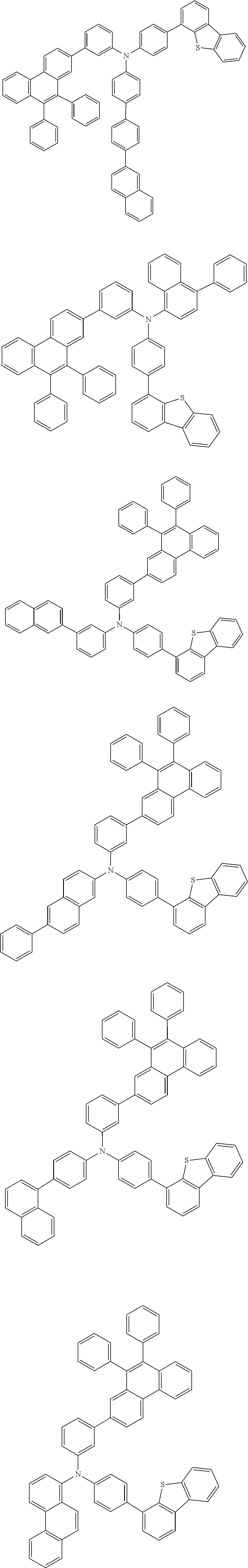

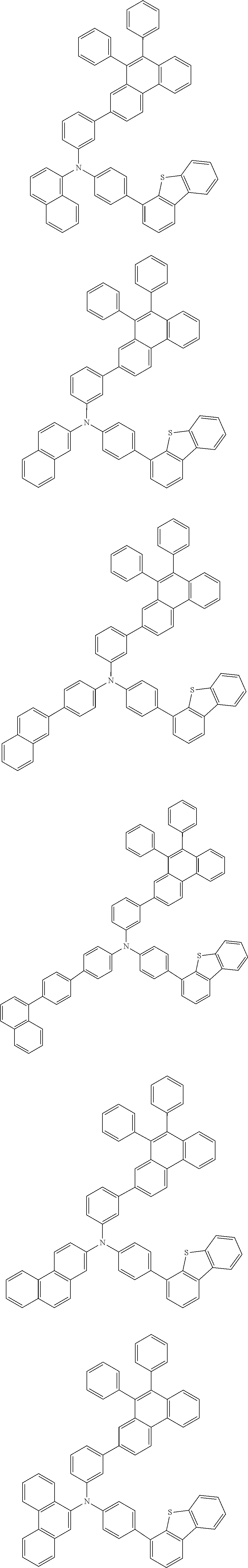

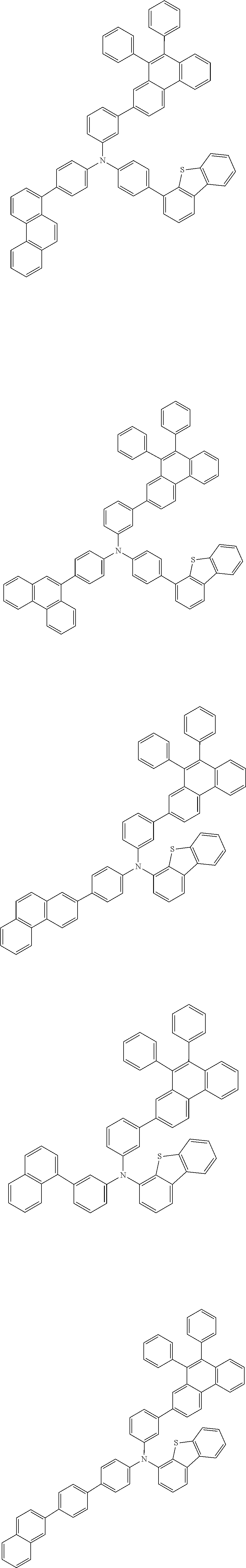

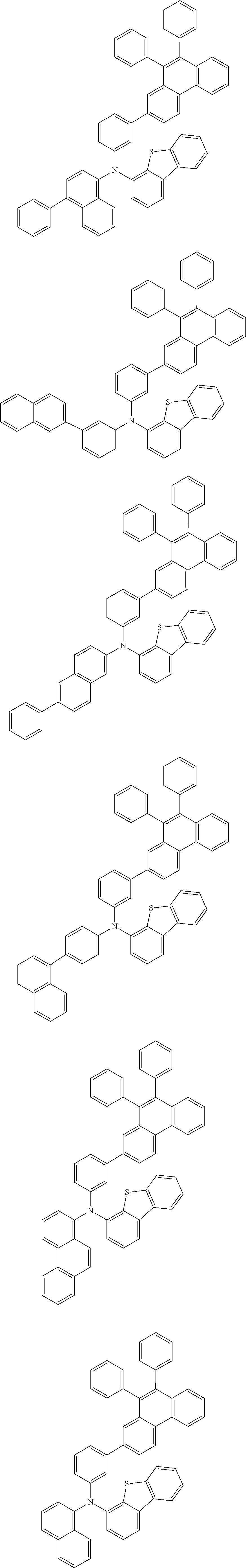

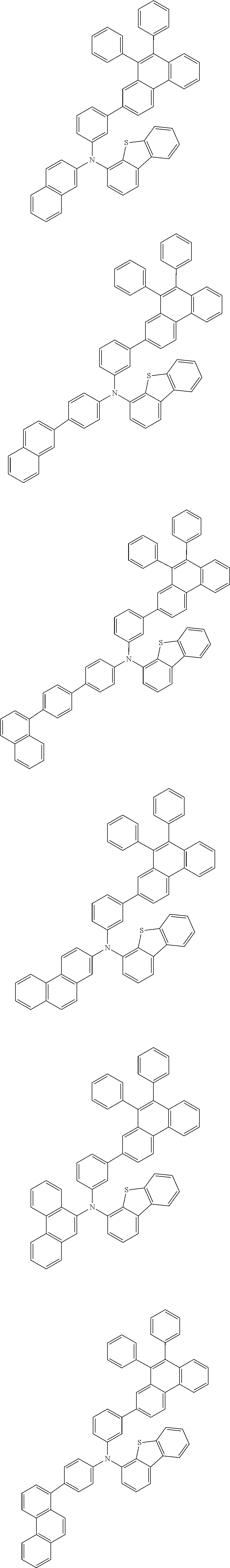

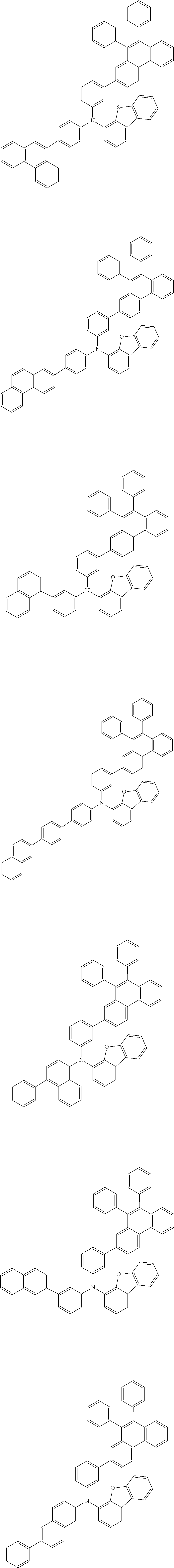

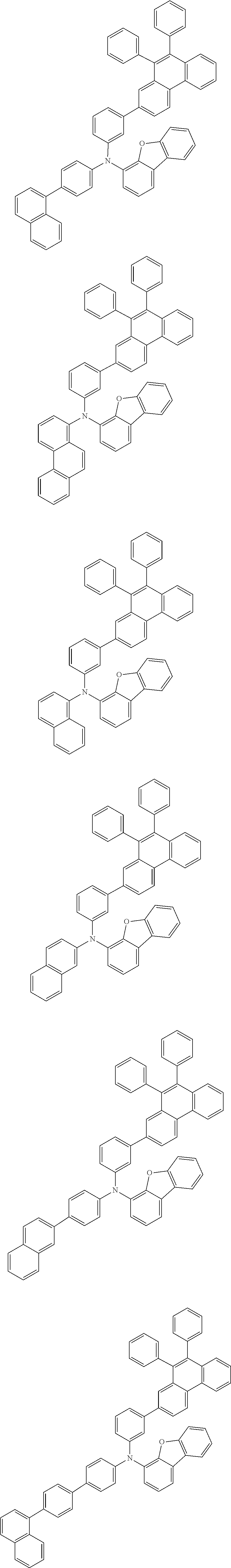

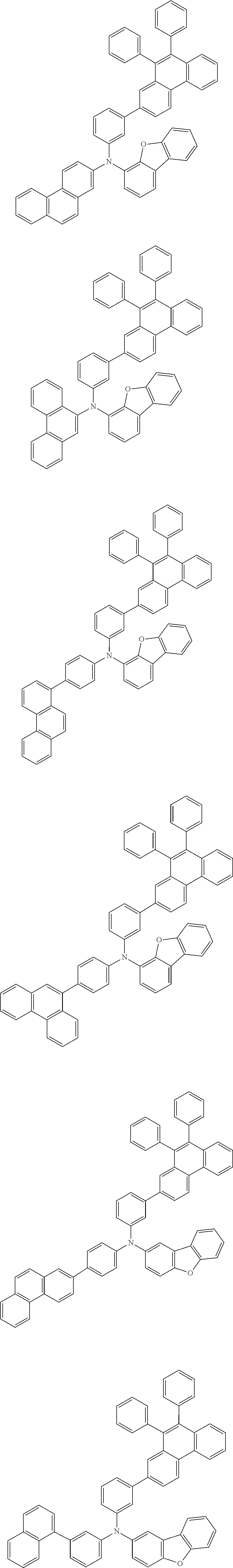

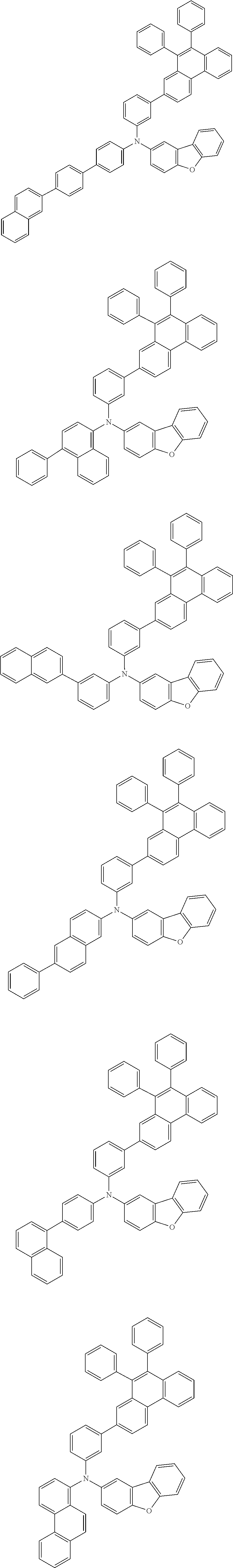

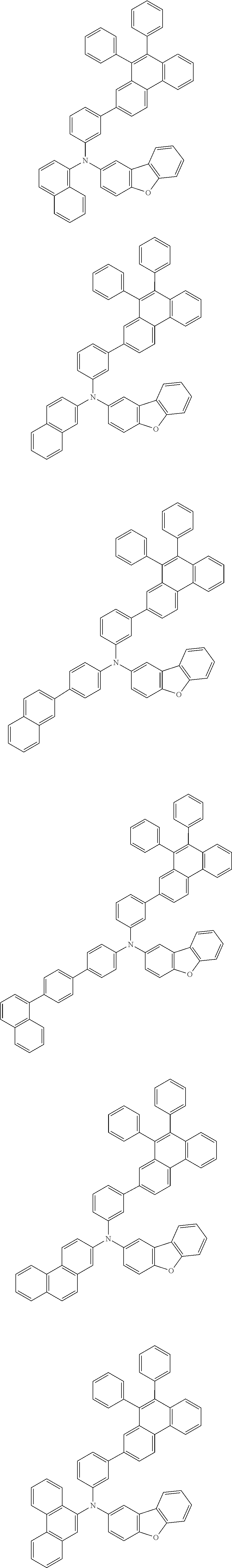

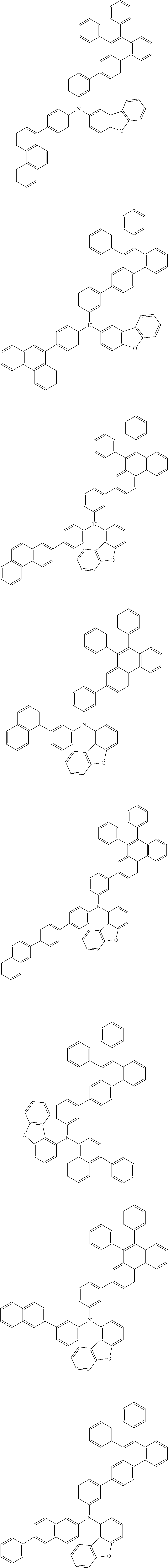

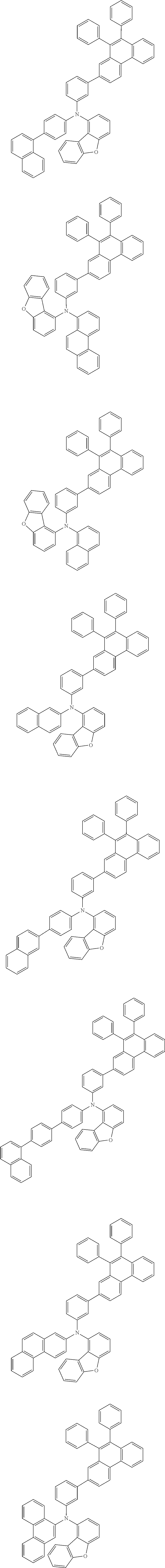

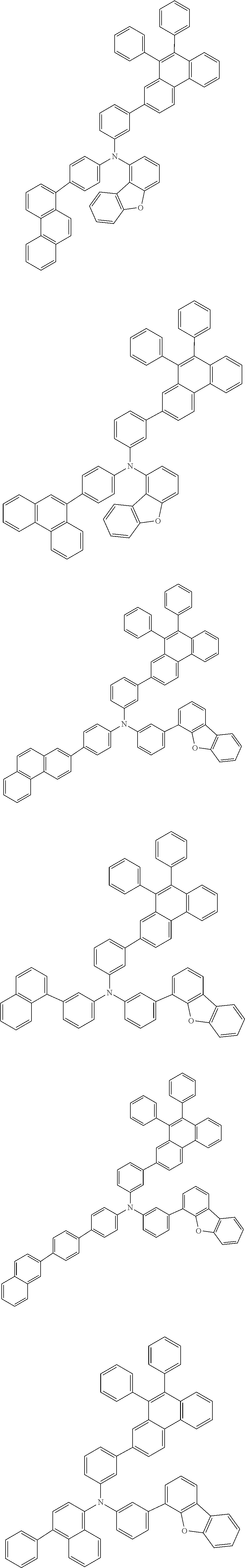

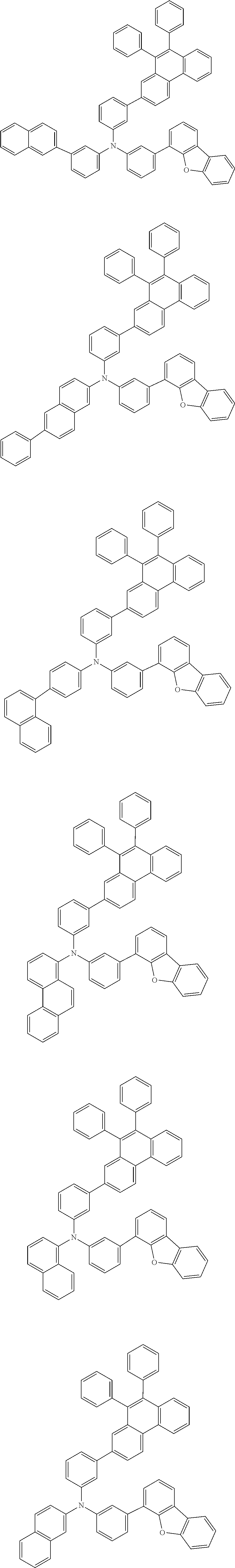

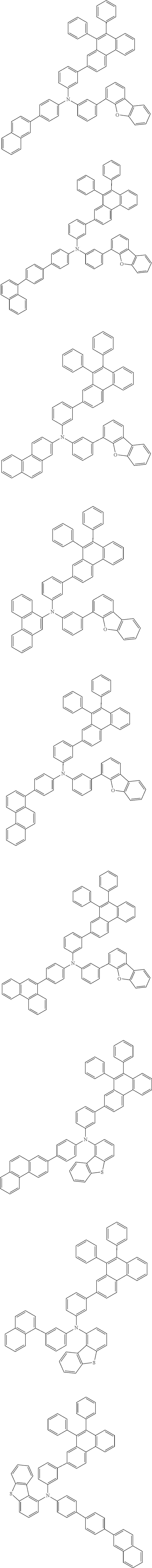

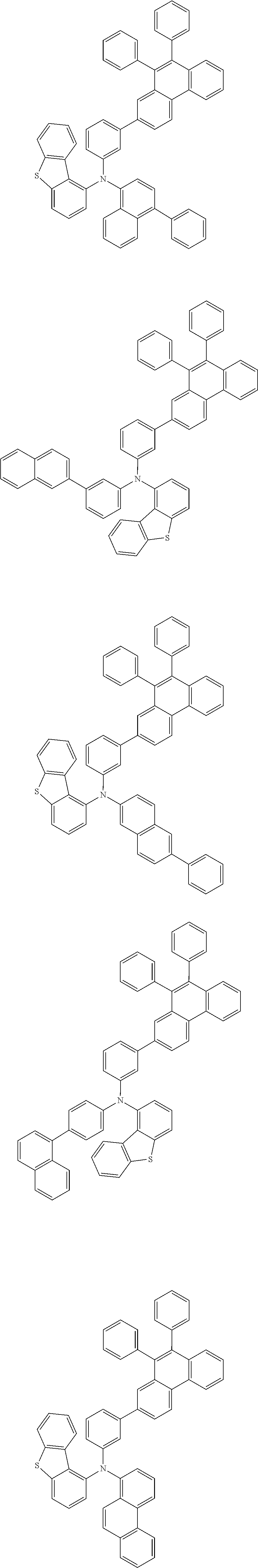

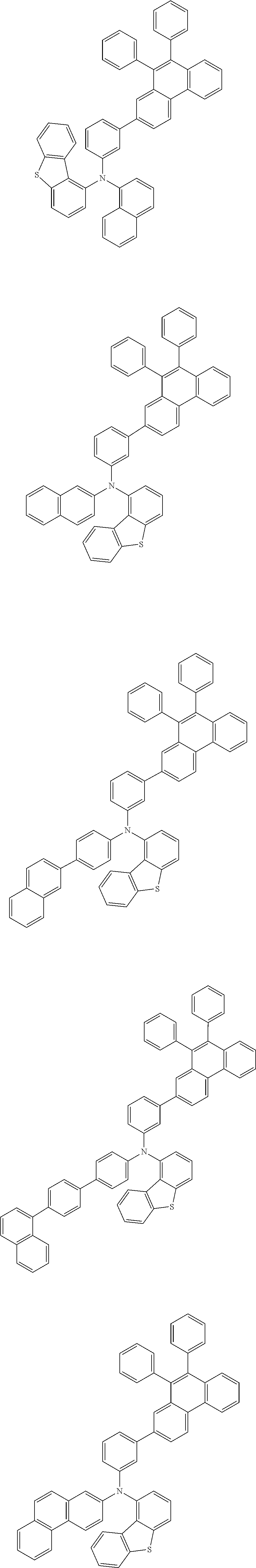

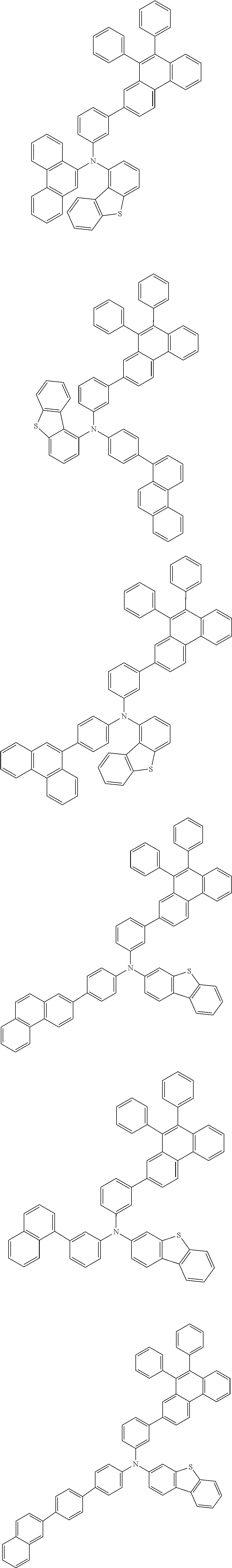

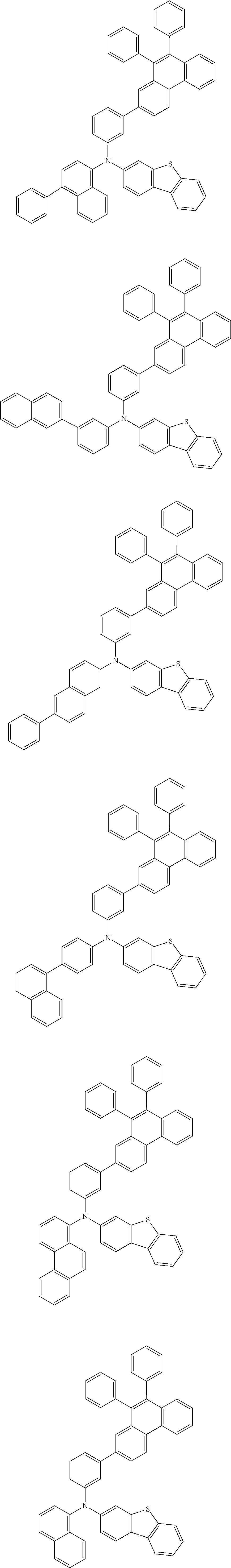

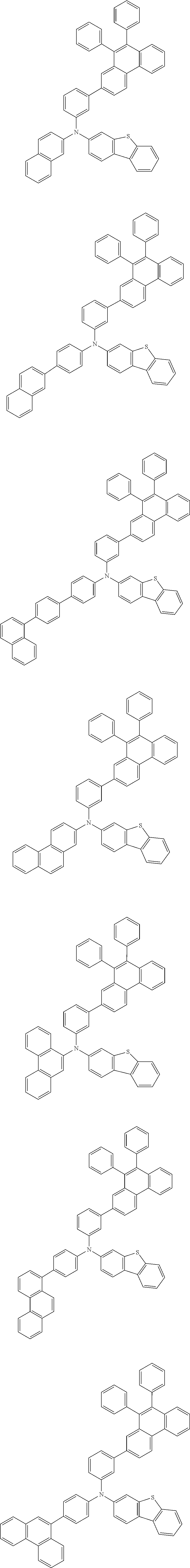

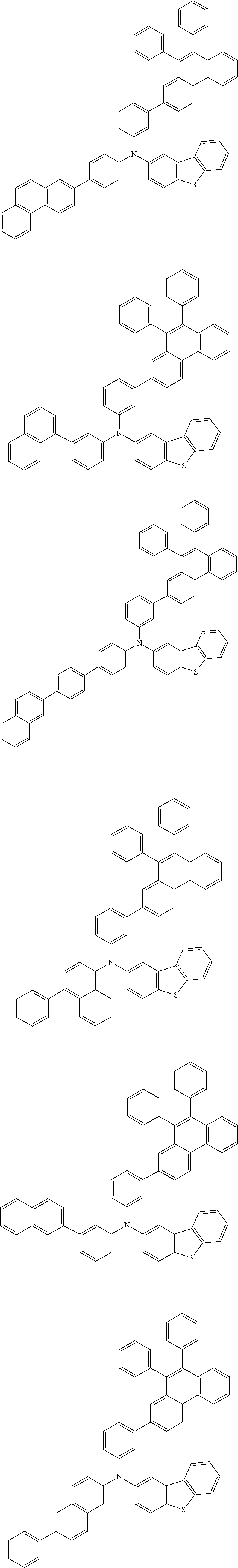

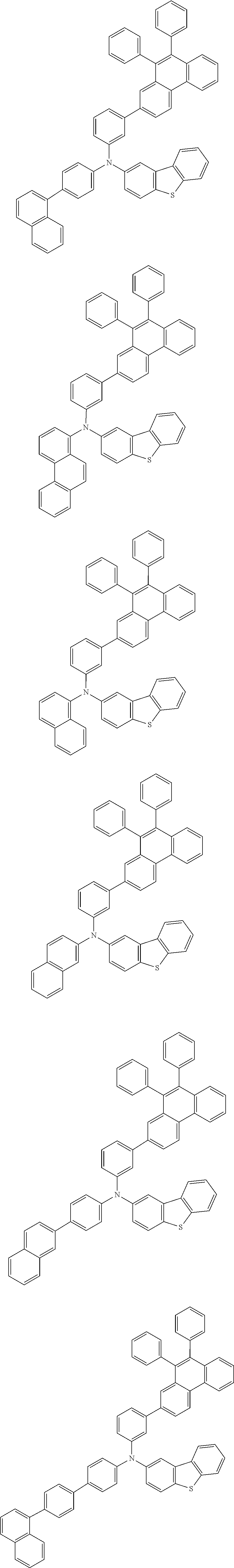

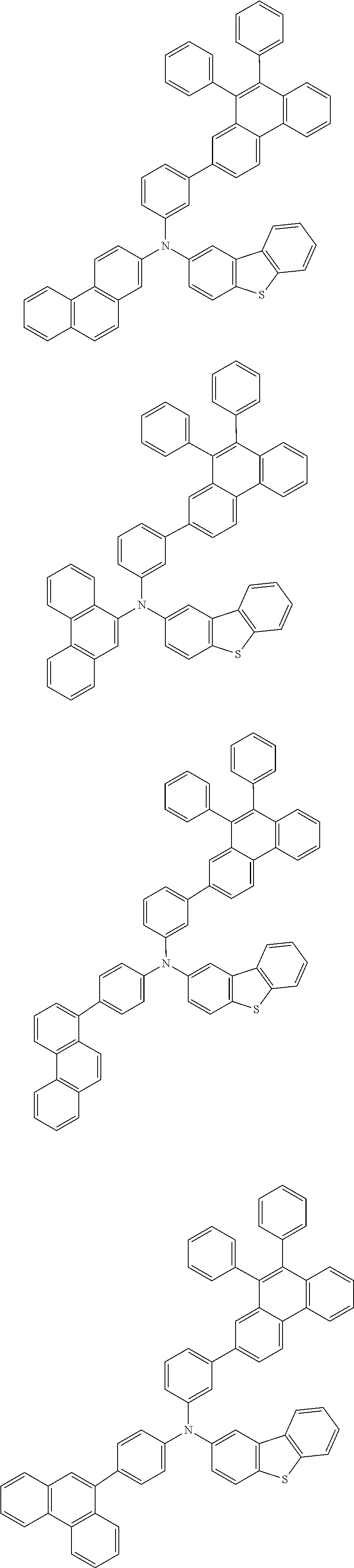 [SMILES: c1ccc(-c2c(-c3ccccc3)c3cc(-c4cccc(N(c5ccc6c(ccc7ccccc76)c5)c5ccc6sc7ccccc7c6c5)c4)ccc3c3ccccc23)cc1 ; c1ccc(-c2c(-c3ccccc3)c3cc(-c4cccc(N(c5ccc6sc7ccccc7c6c5)c5cc6ccccc6c6ccccc56)c4)ccc3c3ccccc23)cc1 ; c1ccc(-c2c(-c3ccccc3)c3cc(-c4cccc(N(c5ccc(-c6cccc7c6ccc6ccccc67)cc5)c5ccc6sc7ccccc7c6c5)c4)ccc3c3ccccc23)cc1 ; c1ccc(-c2c(-c3ccccc3)c3cc(-c4cccc(N(c5ccc(-c6cc7ccccc7c7ccccc67)cc5)c5ccc6sc7ccccc7c6c5)c4)ccc3c3ccccc23)cc1]

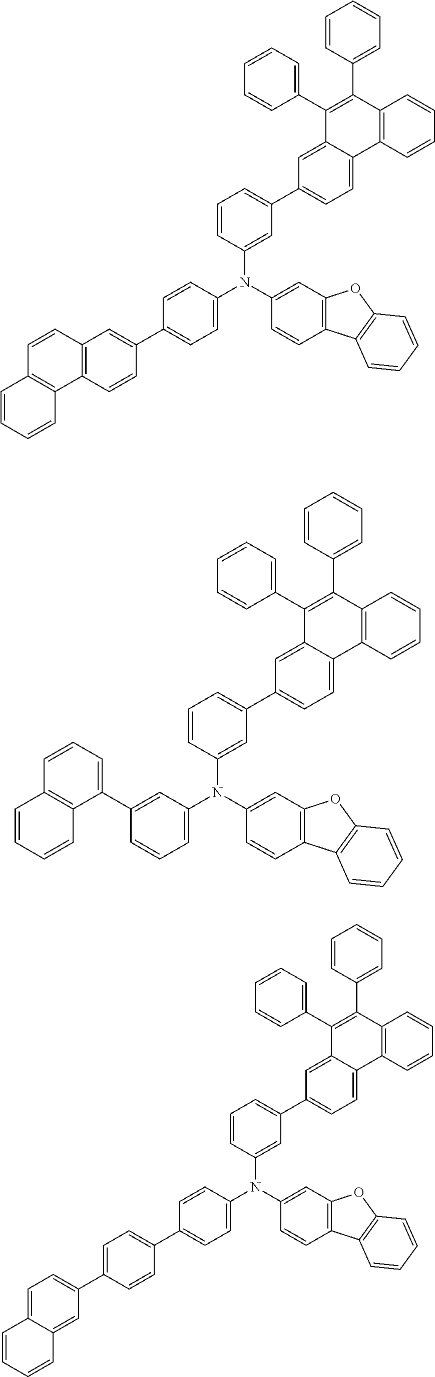

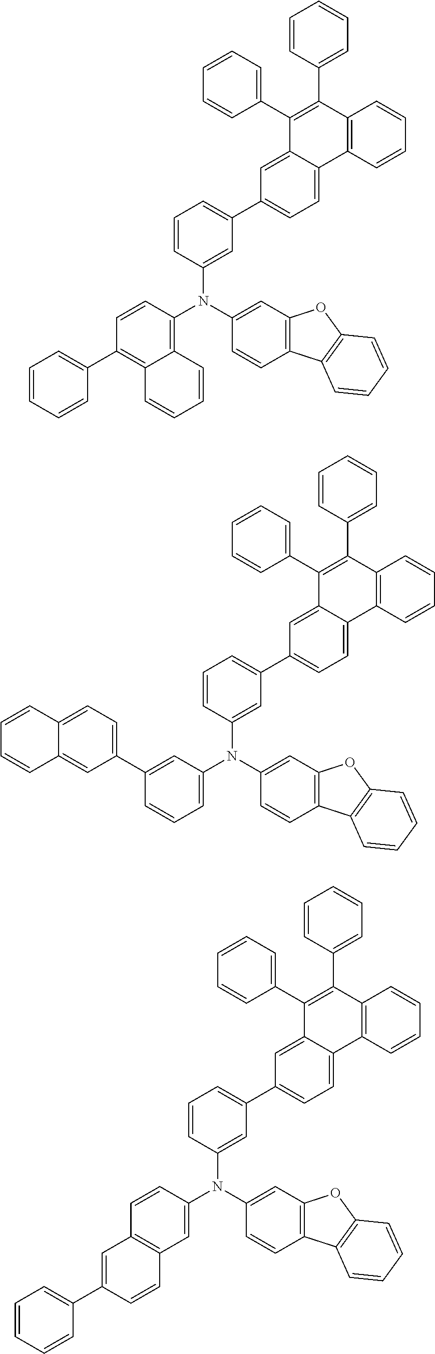 [SMILES: c1ccc(-c2ccc(N(c3cccc(-c4ccc5c(c4)c(-c4ccccc4)c(-c4ccccc4)c4ccccc45)c3)c3ccc4c(c3)oc3ccccc34)c3ccccc23)cc1 ; c1ccc(-c2c(-c3ccccc3)c3cc(-c4cccc(N(c5cccc(-c6ccc7ccccc7c6)c5)c5ccc6c(c5)oc5ccccc56)c4)ccc3c3ccccc23)cc1 ; c1ccc(-c2ccc3cc(N(c4cccc(-c5ccc6c(c5)c(-c5ccccc5)c(-c5ccccc5)c5ccccc56)c4)c4ccc5c(c4)oc4ccccc45)ccc3c2)cc1]

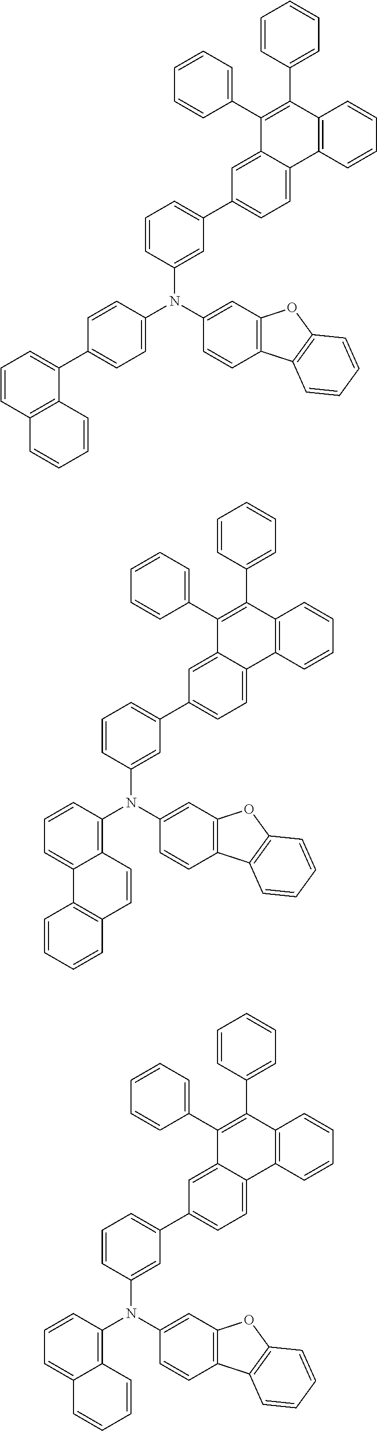 [SMILES: c1ccc(-c2c(-c3ccccc3)c3cc(-c4cccc(N(c5ccc(-c6cccc7ccccc67)cc5)c5ccc6c(c5)oc5ccccc56)c4)ccc3c3ccccc23)cc1 ; c1ccc(-c2c(-c3ccccc3)c3cc(-c4cccc(N(c5ccc6c(c5)oc5ccccc56)c5cccc6c5ccc5ccccc56)c4)ccc3c3ccccc23)cc1 ; c1ccc(-c2c(-c3ccccc3)c3cc(-c4cccc(N(c5ccc6c(c5)oc5ccccc56)c5cccc6ccccc56)c4)ccc3c3ccccc23)cc1]

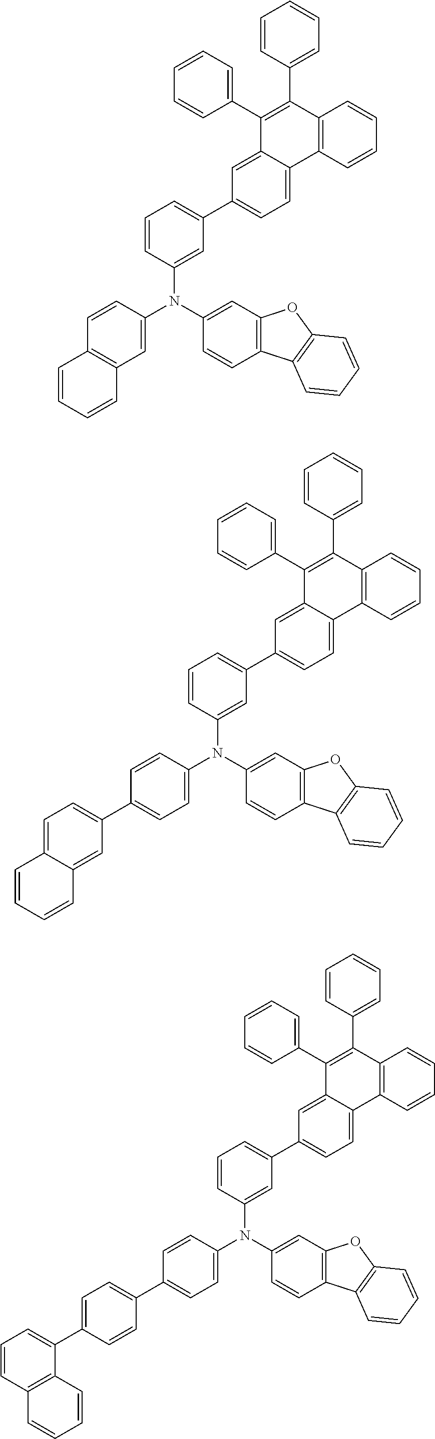 [SMILES: c1ccc(-c2c(-c3ccccc3)c3cc(-c4cccc(N(c5ccc6ccccc6c5)c5ccc6c(c5)oc5ccccc56)c4)ccc3c3ccccc23)cc1 ; c1ccc(-c2c(-c3ccccc3)c3cc(-c4cccc(N(c5ccc(-c6ccc7ccccc7c6)cc5)c5ccc6c(c5)oc5ccccc56)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5ccc6c(c5)oc5ccccc56)c4)ccc3c3ccccc23)cc1]

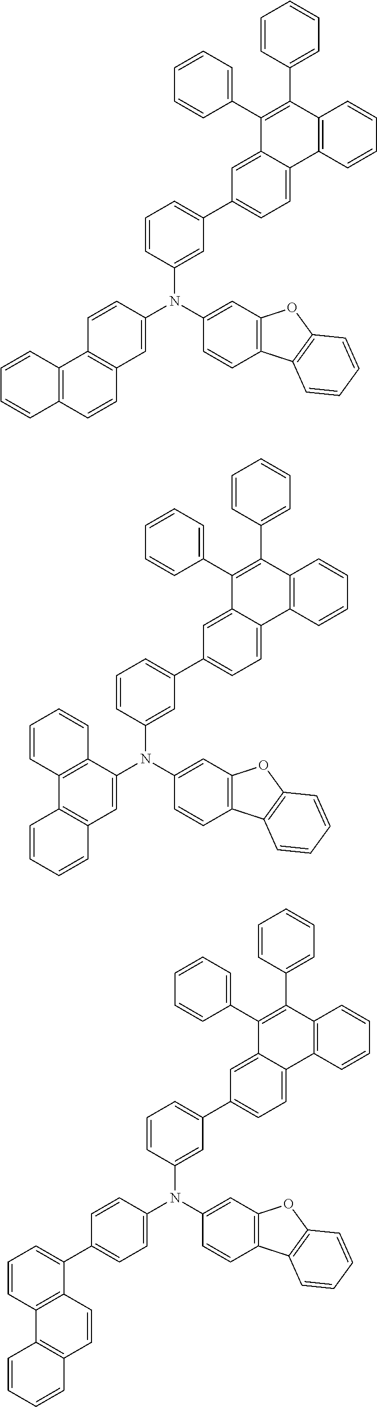 [SMILES: c1ccc(-c2c(-c3ccccc3)c3cc(-c4cccc(N(c5ccc6c(ccc7ccccc76)c5)c5ccc6c(c5)oc5ccccc56)c4)ccc3c3ccccc23)cc1 ; c1ccc(-c2c(-c3ccccc3)c3cc(-c4cccc(N(c5ccc6c(c5)oc5ccccc56)c5cc6ccccc6c6ccccc56)c4)ccc3c3ccccc23)cc1 ; c1ccc(-c2c(-c3ccccc3)c3cc(-c4cccc(N(c5ccc(-c6cccc7c6ccc6ccccc67)cc5)c5ccc6c(c5)oc5ccccc56)c4)ccc3c3ccccc23)cc1]

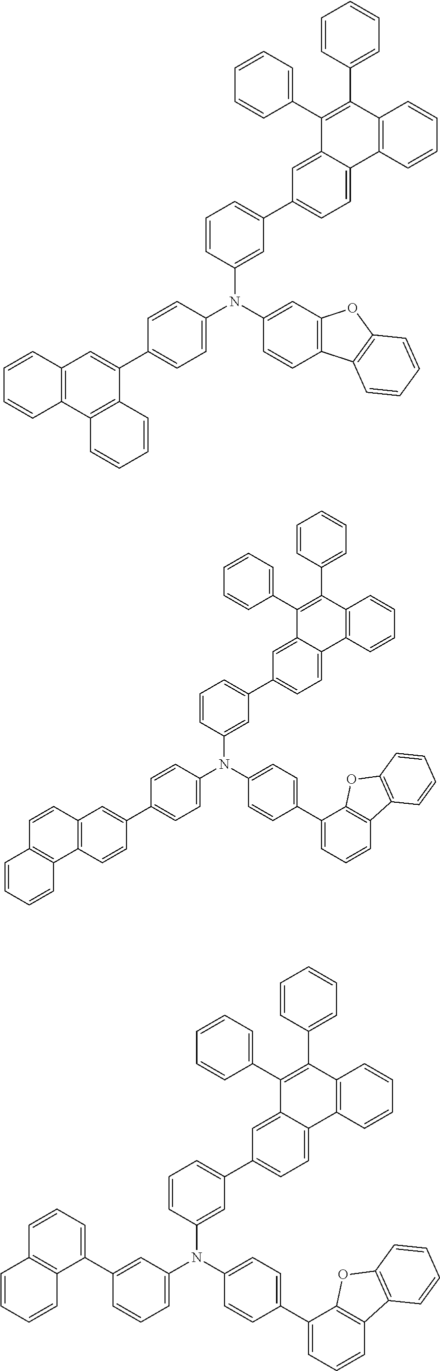 [SMILES: c1ccc(-c2c(-c3ccccc3)c3cc(-c4cccc(N(c5ccc(-c6cc7ccccc7c7ccccc67)cc5)c5ccc6c(c5)oc5ccccc56)c4)ccc3c3ccccc23)cc1 ; c1ccc(-c2c(-c3ccccc3)c3cc(-c4cccc(N(c5ccc(-c6ccc7c(ccc8ccccc87)c6)cc5)c5ccc(-c6cccc7c6oc6ccccc67)cc5)c4)ccc3c3ccccc23)cc1 ; c1ccc(-c2c(-c3ccccc3)c3cc(-c4cccc(N(c5ccc(-c6cccc7c6oc6ccccc67)cc5)c5cccc(-c6cccc7ccccc67)c5)c4)ccc3c3ccccc23)cc1]

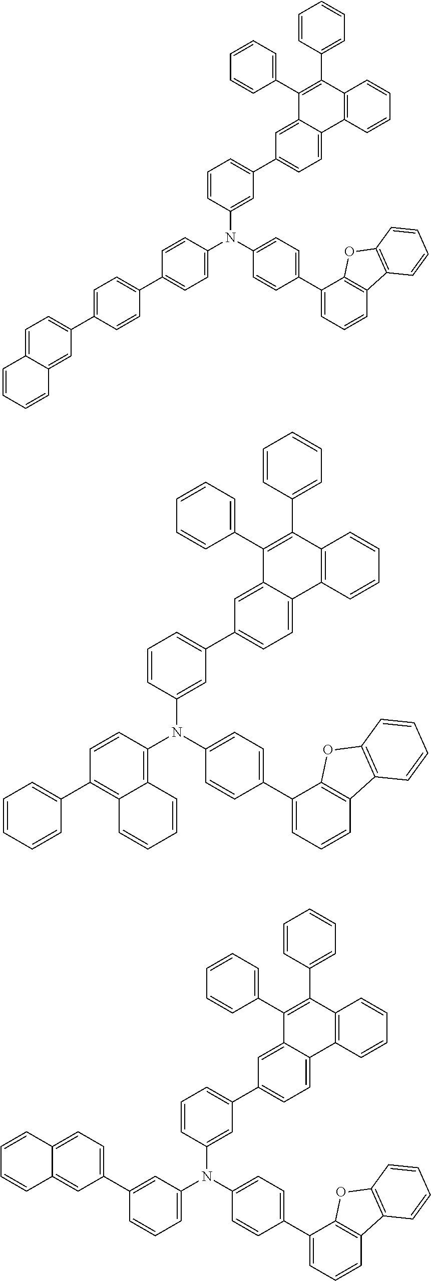 [SMILES: c1ccc(-c2c(-c3ccccc3)c3cc(-c4cccc(N(c5ccc(-c6ccc(-c7ccc8ccccc8c7)cc6)cc5)c5ccc(-c6cccc7c6oc6ccccc67)cc5)c4)ccc3c3ccccc23)cc1 ; c1ccc(-c2ccc(N(c3ccc(-c4cccc5c4oc4ccccc45)cc3)c3cccc(-c4ccc5c(c4)c(-c4ccccc4)c(-c4ccccc4)c4ccccc45)c3)c3ccccc23)cc1 ; c1ccc(-c2c(-c3ccccc3)c3cc(-c4cccc(N(c5ccc(-c6cccc7c6oc6ccccc67)cc5)c5cccc(-c6ccc7ccccc7c6)c5)c4)ccc3c3ccccc23)cc1]

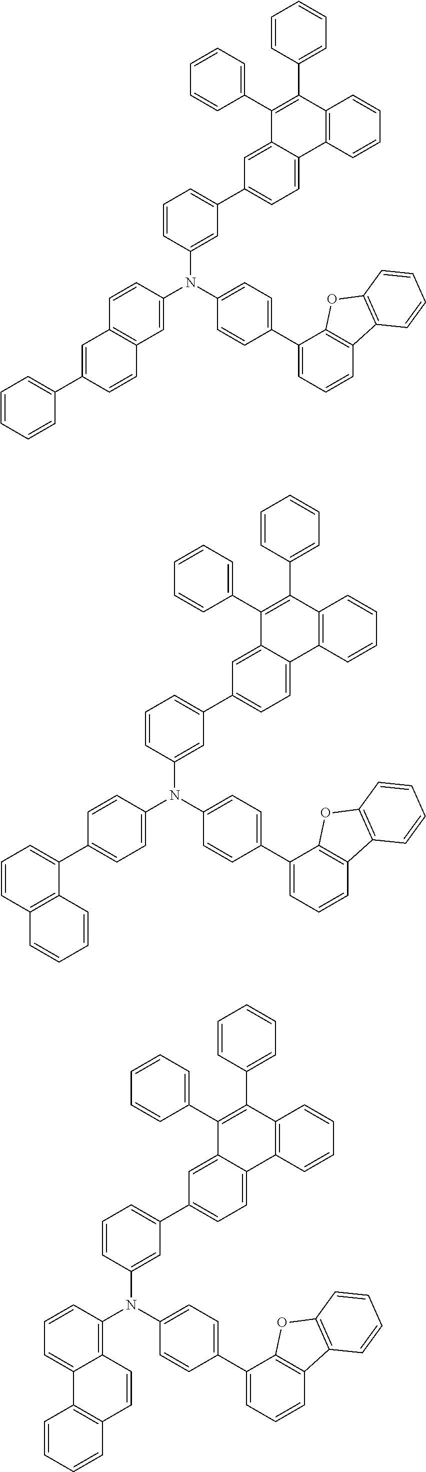 [SMILES: c1ccc(-c2ccc3cc(N(c4ccc(-c5cccc6c5oc5ccccc56)cc4)c4cccc(-c5ccc6c(c5)c(-c5ccccc5)c(-c5ccccc5)c5ccccc56)c4)ccc3c2)cc1 ; c1ccc(-c2c(-c3ccccc3)c3cc(-c4cccc(N(c5ccc(-c6cccc7ccccc67)cc5)c5ccc(-c6cccc7c6oc6ccccc67)cc5)c4)ccc3c3ccccc23)cc1 ; c1ccc(-c2c(-c3ccccc3)c3cc(-c4cccc(N(c5ccc(-c6cccc7c6oc6ccccc67)cc5)c5cccc6c5ccc5ccccc56)c4)ccc3c3ccccc23)cc1]

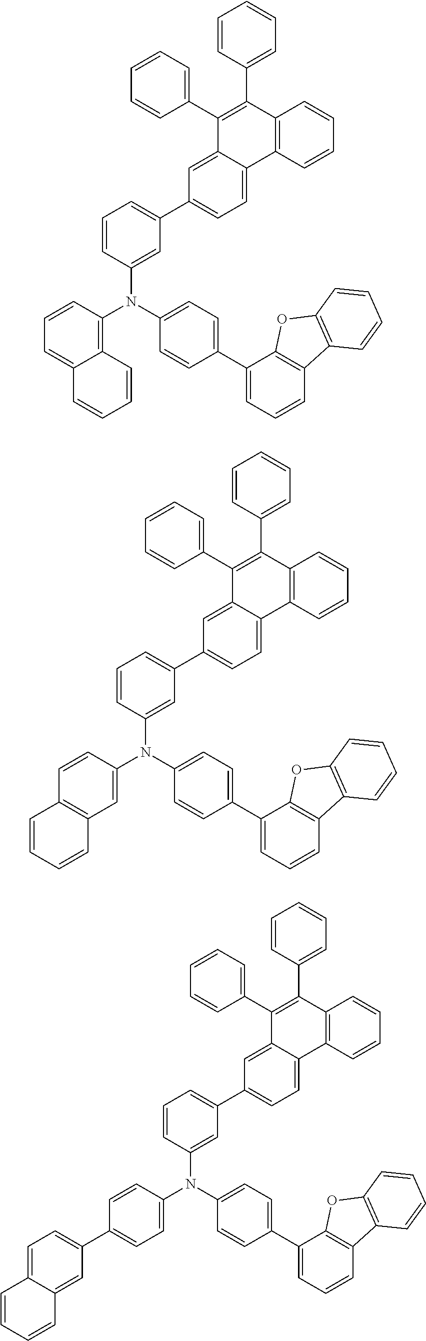

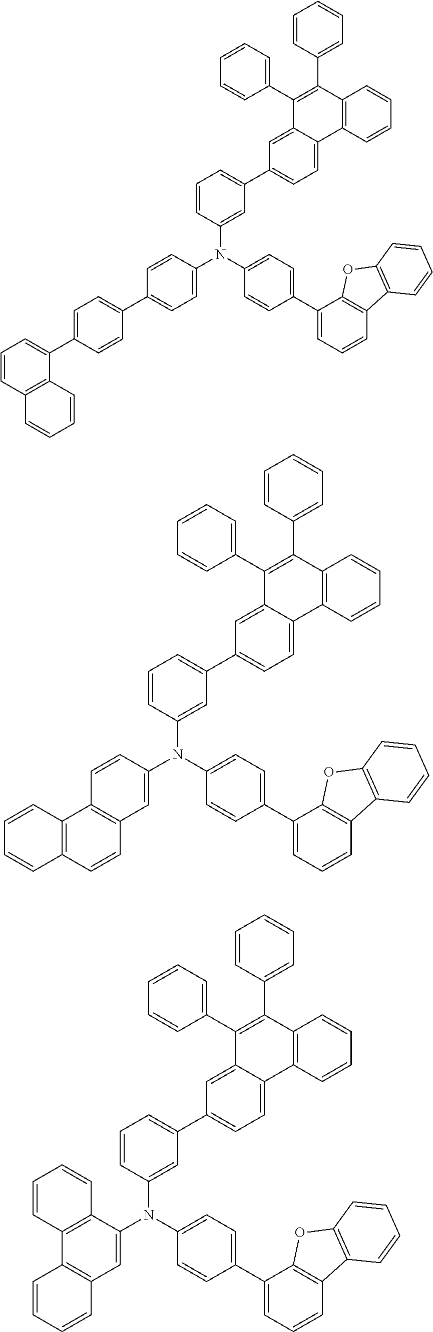 [SMILES: c1ccc(-c2c(-c3ccccc3)c3cc(-c4cccc(N(c5ccc(-c6ccc(-c7cccc8ccccc78)cc6)cc5)c5ccc(-c6cccc7c6oc6ccccc67)cc5)c4)ccc3c3ccccc23)cc1 ; c1ccc(-c2c(-c3ccccc3)c3cc(-c4cccc(N(c5ccc(-c6cccc7c6oc6ccccc67)cc5)c5ccc6c(ccc7ccccc76)c5)c4)ccc3c3ccccc23)cc1 ; c1ccc(-c2c(-c3ccccc3)c3cc(-c4cccc(N(c5ccc(-c6cccc7c6oc6ccccc67)cc5)c5cc6ccccc6c6ccccc56)c4)ccc3c3ccccc23)cc1]

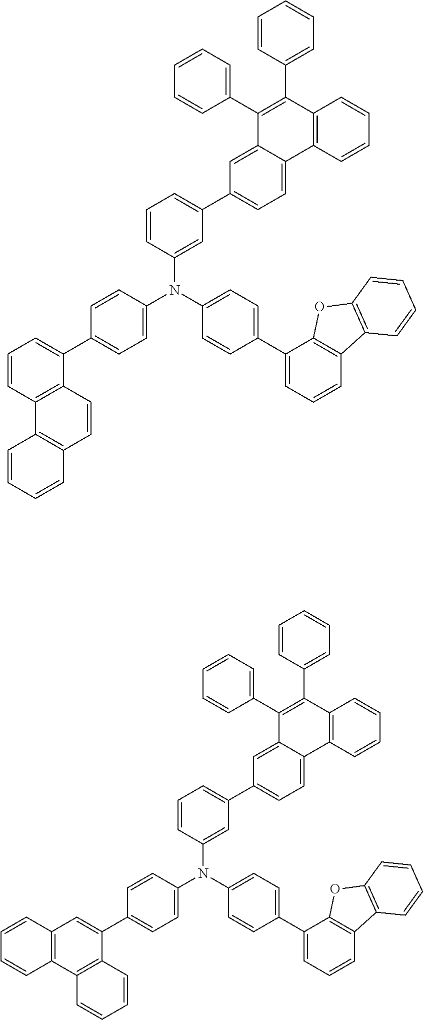 [SMILES: c1ccc(-c2c(-c3ccccc3)c3cc(-c4cccc(N(c5ccc(-c6cccc7c6ccc6ccccc67)cc5)c5ccc(-c6cccc7c6oc6ccccc67)cc5)c4)ccc3c3ccccc23)cc1 ; c1ccc(-c2c(-c3ccccc3)c3cc(-c4cccc(N(c5ccc(-c6cc7ccccc7c7ccccc67)cc5)c5ccc(-c6cccc7c6oc6ccccc67)cc5)c4)ccc3c3ccccc23)cc1]

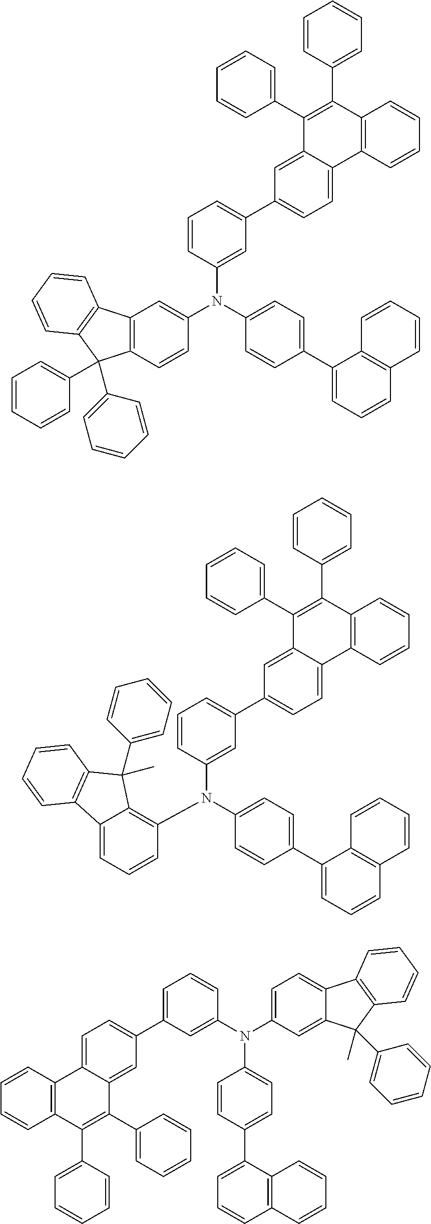

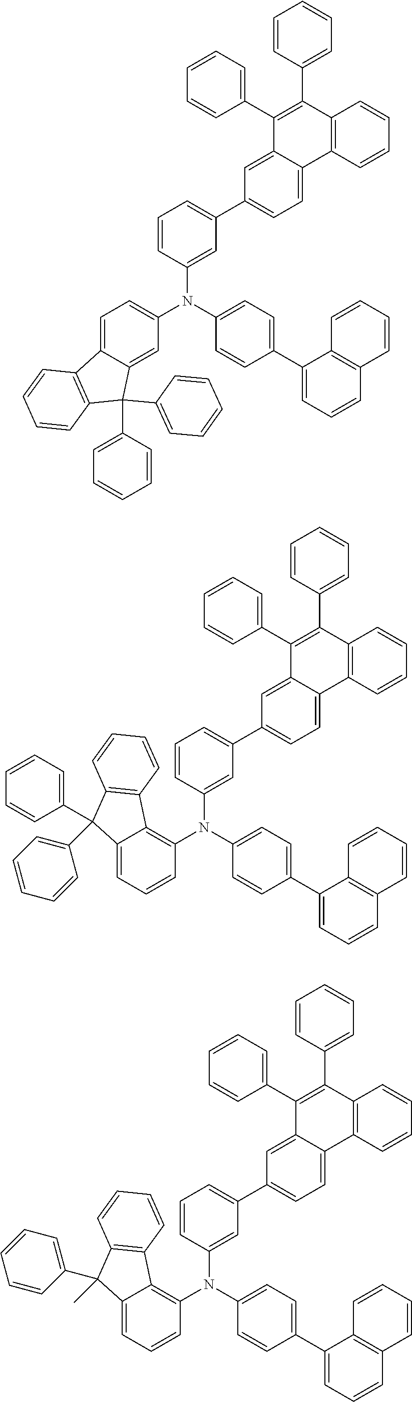 [SMILES: c1ccc(-c2c(-c3ccccc3)c3cc(-c4cccc(N(c5ccc(-c6cccc7ccccc67)cc5)c5ccc6c(c5)C(c5ccccc5)(c5ccccc5)c5ccccc5-6)c4)ccc3c3ccccc23)cc1 ; c1ccc(-c2c(-c3ccccc3)c3cc(-c4cccc(N(c5ccc(-c6cccc7ccccc67)cc5)c5cccc6c5-c5ccccc5C6(c5ccccc5)c5ccccc5)c4)ccc3c3ccccc23)cc1 ; CC1(c2ccccc2)c2ccccc2-c2c(N(c3ccc(-c4cccc5ccccc45)cc3)c3cccc(-c4ccc5c(c4)c(-c4ccccc4)c(-c4ccccc4)c4ccccc45)c3)cccc21]

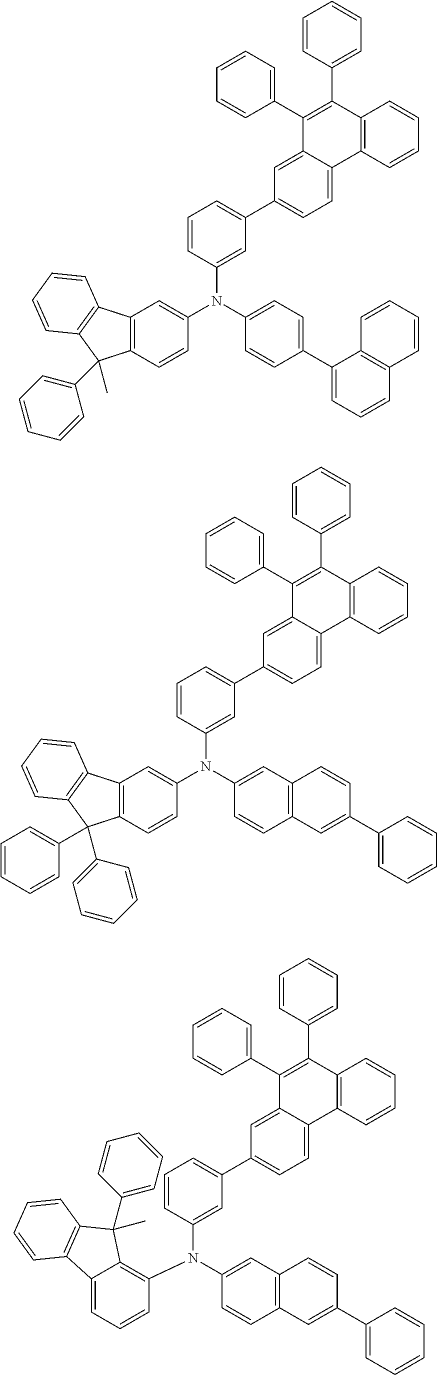 [SMILES: CC1(c2ccccc2)c2ccccc2-c2cc(N(c3ccc(-c4cccc5ccccc45)cc3)c3cccc(-c4ccc5c(c4)c(-c4ccccc4)c(-c4ccccc4)c4ccccc45)c3)ccc21 ; c1ccc(-c2ccc3cc(N(c4cccc(-c5ccc6c(c5)c(-c5ccccc5)c(-c5ccccc5)c5ccccc56)c4)c4ccc5c(c4)-c4ccccc4C5(c4ccccc4)c4ccccc4)ccc3c2)cc1 ; CC1(c2ccccc2)c2ccccc2-c2cccc(N(c3cccc(-c4ccc5c(c4)c(-c4ccccc4)c(-c4ccccc4)c4ccccc45)c3)c3ccc4cc(-c5ccccc5)ccc4c3)c21]

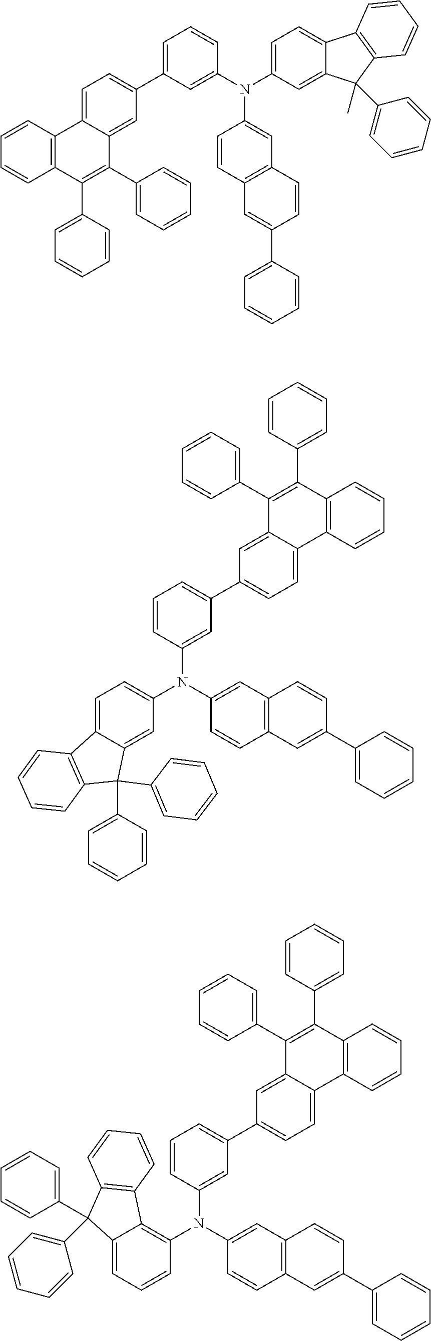 [SMILES: CC1(c2ccccc2)c2ccccc2-c2ccc(N(c3cccc(-c4ccc5c(c4)c(-c4ccccc4)c(-c4ccccc4)c4ccccc45)c3)c3ccc4cc(-c5ccccc5)ccc4c3)cc21 ; c1ccc(-c2ccc3cc(N(c4cccc(-c5ccc6c(c5)c(-c5ccccc5)c(-c5ccccc5)c5ccccc56)c4)c4ccc5c(c4)C(c4ccccc4)(c4ccccc4)c4ccccc4-5)ccc3c2)cc1 ; c1ccc(-c2ccc3cc(N(c4cccc(-c5ccc6c(c5)c(-c5ccccc5)c(-c5ccccc5)c5ccccc56)c4)c4cccc5c4-c4ccccc4C5(c4ccccc4)c4ccccc4)ccc3c2)cc1]

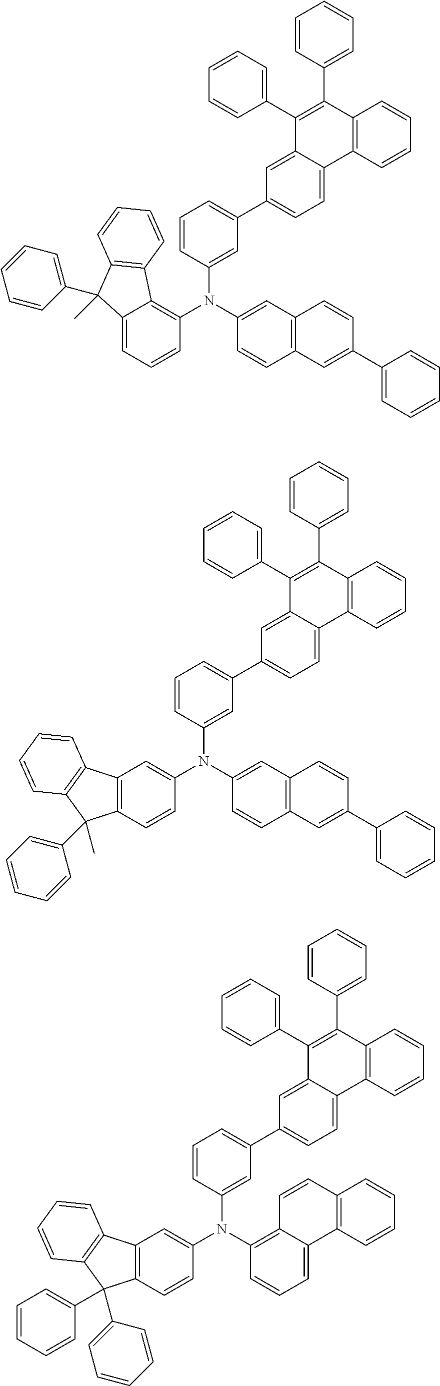 [SMILES: CC1(c2ccccc2)c2ccccc2-c2c(N(c3cccc(-c4ccc5c(c4)c(-c4ccccc4)c(-c4ccccc4)c4ccccc45)c3)c3ccc4cc(-c5ccccc5)ccc4c3)cccc21 ; CC1(c2ccccc2)c2ccccc2-c2cc(N(c3cccc(-c4ccc5c(c4)c(-c4ccccc4)c(-c4ccccc4)c4ccccc45)c3)c3ccc4cc(-c5ccccc5)ccc4c3)ccc21 ; c1ccc(-c2c(-c3ccccc3)c3cc(-c4cccc(N(c5ccc6c(c5)-c5ccccc5C6(c5ccccc5)c5ccccc5)c5cccc6c5ccc5ccccc56)c4)ccc3c3ccccc23)cc1]

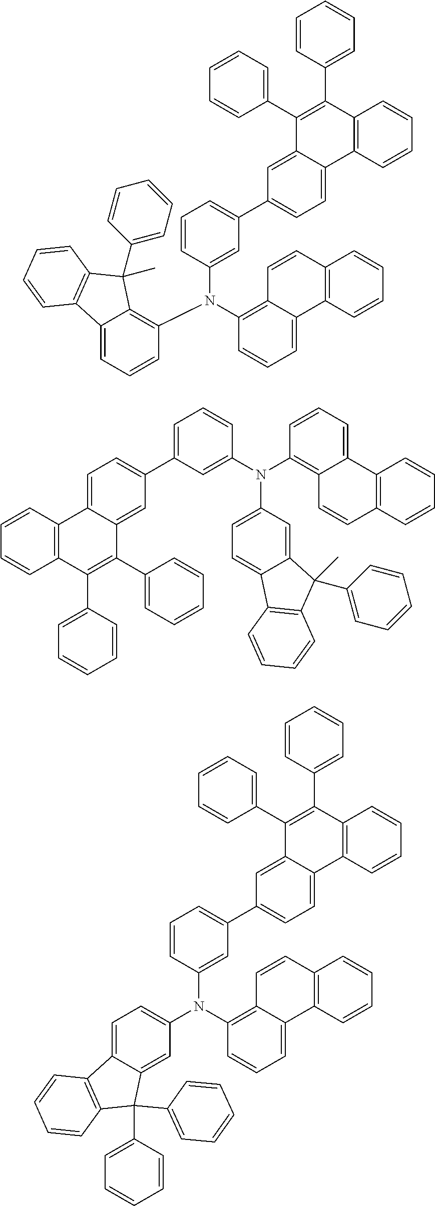

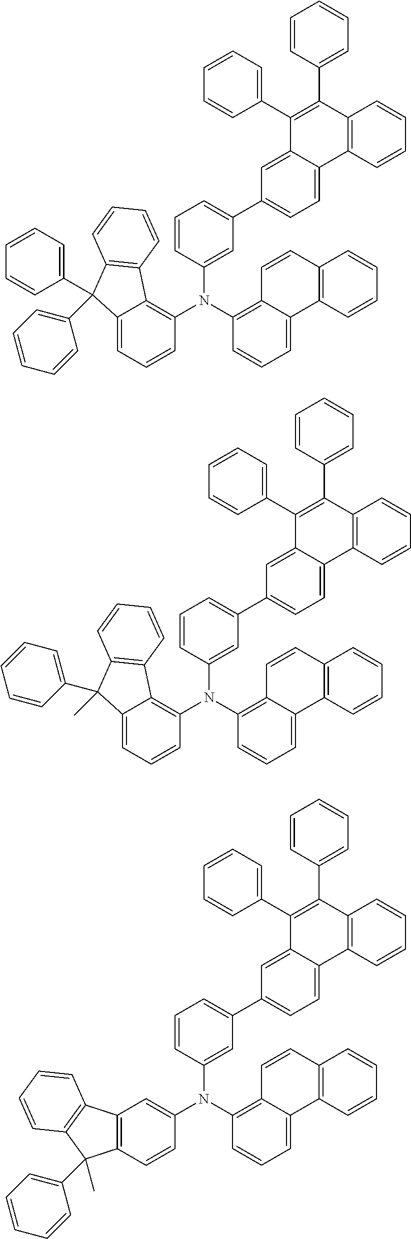 [SMILES: c1ccc(-c2c(-c3ccccc3)c3cc(-c4cccc(N(c5cccc6c5-c5ccccc5C6(c5ccccc5)c5ccccc5)c5cccc6c5ccc5ccccc56)c4)ccc3c3ccccc23)cc1 ; CC1(c2ccccc2)c2ccccc2-c2c(N(c3cccc(-c4ccc5c(c4)c(-c4ccccc4)c(-c4ccccc4)c4ccccc45)c3)c3cccc4c3ccc3ccccc34)cccc21 ; CC1(c2ccccc2)c2ccccc2-c2cc(N(c3cccc(-c4ccc5c(c4)c(-c4ccccc4)c(-c4ccccc4)c4ccccc45)c3)c3cccc4c3ccc3ccccc34)ccc21]

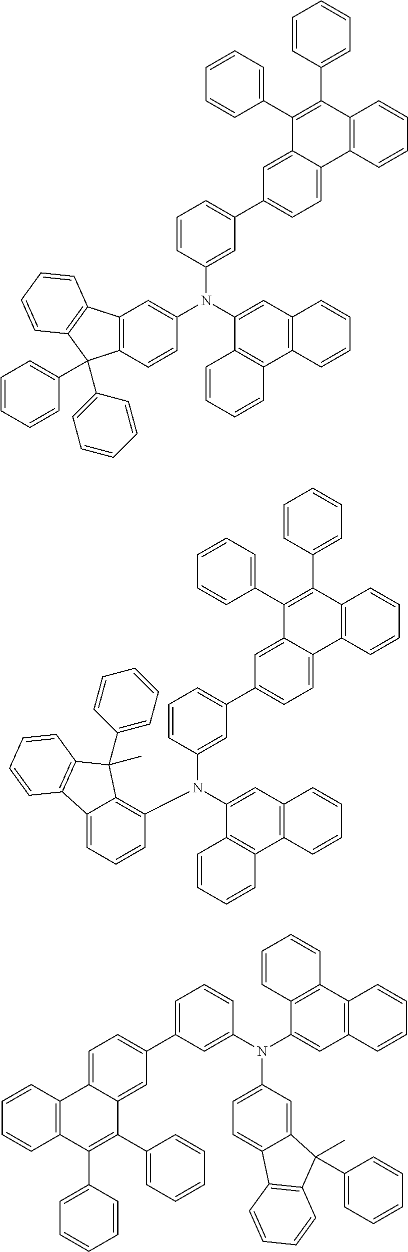 [SMILES: c1ccc(-c2c(-c3ccccc3)c3cc(-c4cccc(N(c5ccc6c(c5)-c5ccccc5C6(c5ccccc5)c5ccccc5)c5cc6ccccc6c6ccccc56)c4)ccc3c3ccccc23)cc1 ; CC1(c2ccccc2)c2ccccc2-c2cccc(N(c3cccc(-c4ccc5c(c4)c(-c4ccccc4)c(-c4ccccc4)c4ccccc45)c3)c3cc4ccccc4c4ccccc34)c21 ; CC1(c2ccccc2)c2ccccc2-c2ccc(N(c3cccc(-c4ccc5c(c4)c(-c4ccccc4)c(-c4ccccc4)c4ccccc45)c3)c3cc4ccccc4c4ccccc34)cc21]

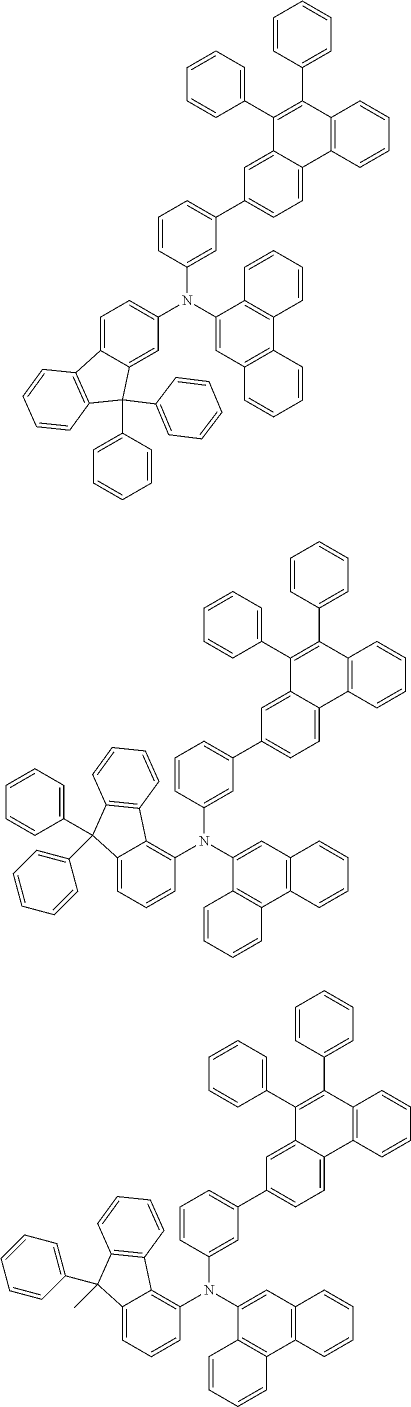 [SMILES: c1ccc(-c2c(-c3ccccc3)c3cc(-c4cccc(N(c5ccc6c(c5)C(c5ccccc5)(c5ccccc5)c5ccccc5-6)c5cc6ccccc6c6ccccc56)c4)ccc3c3ccccc23)cc1 ; c1ccc(-c2c(-c3ccccc3)c3cc(-c4cccc(N(c5cccc6c5-c5ccccc5C6(c5ccccc5)c5ccccc5)c5cc6ccccc6c6ccccc56)c4)ccc3c3ccccc23)cc1 ; CC1(c2ccccc2)c2ccccc2-c2c(N(c3cccc(-c4ccc5c(c4)c(-c4ccccc4)c(-c4ccccc4)c4ccccc45)c3)c3cc4ccccc4c4ccccc34)cccc21]

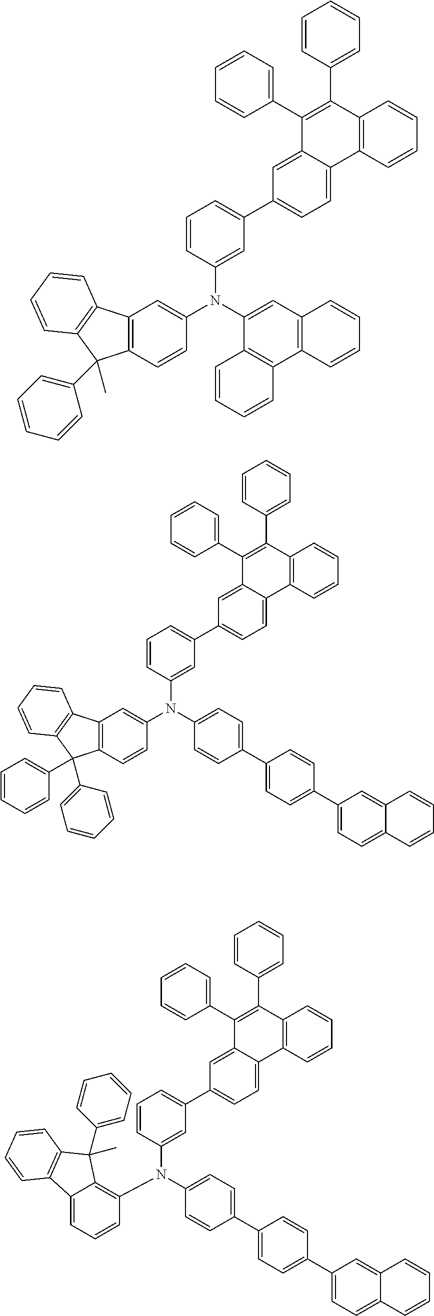 [SMILES: CC1(c2ccccc2)c2ccccc2-c2cc(N(c3cccc(-c4ccc5c(c4)c(-c4ccccc4)c(-c4ccccc4)c4ccccc45)c3)c3cc4ccccc4c4ccccc34)ccc21 ; c1ccc(-c2c(-c3ccccc3)c3cc(-c4cccc(N(c5ccc(-c6ccc(-c7ccc8ccccc8c7)cc6)cc5)c5ccc6c(c5)-c5ccccc5C6(c5ccccc5)c5ccccc5)c4)ccc3c3ccccc23)cc1 ; CC1(c2ccccc2)c2ccccc2-c2cccc(N(c3ccc(-c4ccc(-c5ccc6ccccc6c5)cc4)cc3)c3cccc(-c4ccc5c(c4)c(-c4ccccc4)c(-c4ccccc4)c4ccccc45)c3)c21]

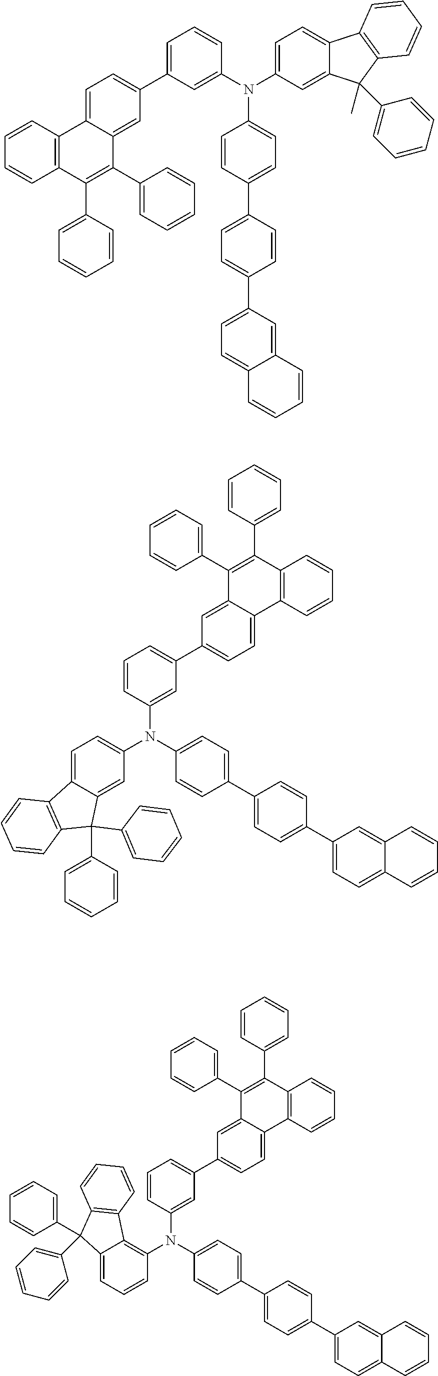 [SMILES: CC1(c2ccccc2)c2ccccc2-c2ccc(N(c3ccc(-c4ccc(-c5ccc6ccccc6c5)cc4)cc3)c3cccc(-c4ccc5c(c4)c(-c4ccccc4)c(-c4ccccc4)c4ccccc45)c3)cc21 ; c1ccc(-c2c(-c3ccccc3)c3cc(-c4cccc(N(c5ccc(-c6ccc(-c7ccc8ccccc8c7)cc6)cc5)c5ccc6c(c5)C(c5ccccc5)(c5ccccc5)c5ccccc5-6)c4)ccc3c3ccccc23)cc1 ; c1ccc(-c2c(-c3ccccc3)c3cc(-c4cccc(N(c5ccc(-c6ccc(-c7ccc8ccccc8c7)cc6)cc5)c5cccc6c5-c5ccccc5C6(c5ccccc5)c5ccccc5)c4)ccc3c3ccccc23)cc1]

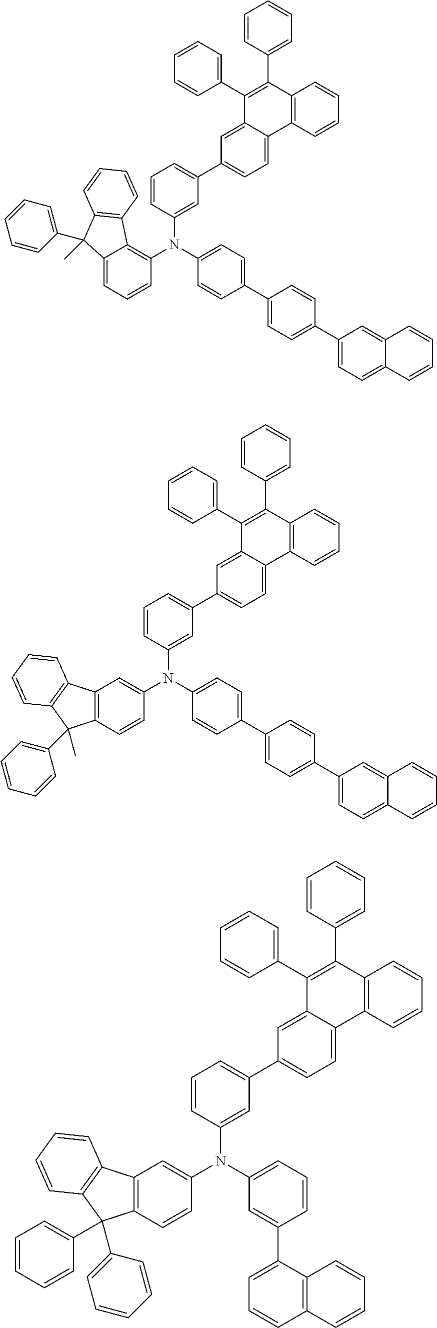 [SMILES: CC1(c2ccccc2)c2ccccc2-c2c(N(c3ccc(-c4ccc(-c5ccc6ccccc6c5)cc4)cc3)c3cccc(-c4ccc5c(c4)c(-c4ccccc4)c(-c4ccccc4)c4ccccc45)c3)cccc21 ; CC1(c2ccccc2)c2ccccc2-c2cc(N(c3ccc(-c4ccc(-c5ccc6ccccc6c5)cc4)cc3)c3cccc(-c4ccc5c(c4)c(-c4ccccc4)c(-c4ccccc4)c4ccccc45)c3)ccc21 ; c1ccc(-c2c(-c3ccccc3)c3cc(-c4cccc(N(c5cccc(-c6cccc7ccccc67)c5)c5ccc6c(c5)-c5ccccc5C6(c5ccccc5)c5ccccc5)c4)ccc3c3ccccc23)cc1]

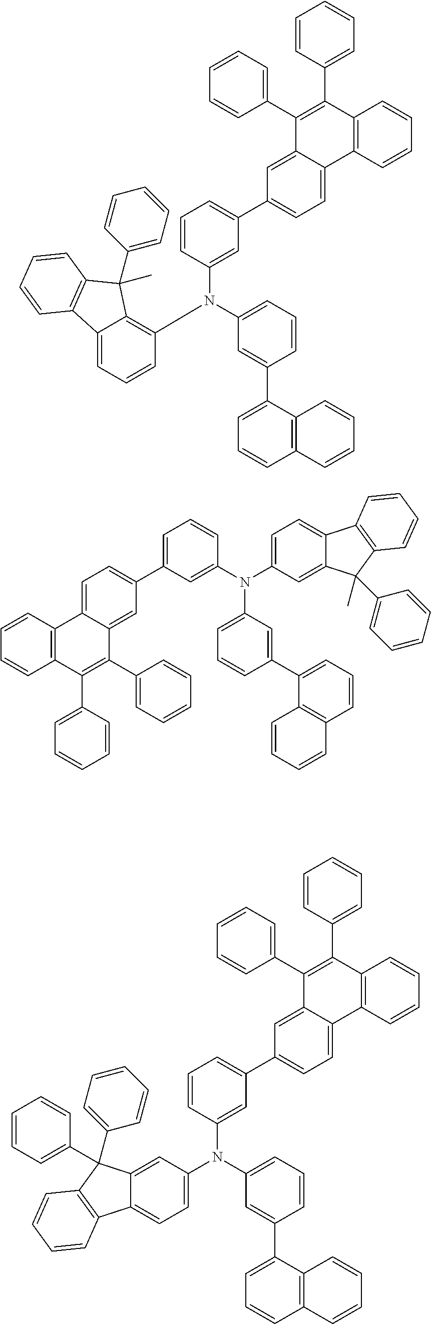 [SMILES: CC1(c2ccccc2)c2ccccc2-c2cccc(N(c3cccc(-c4ccc5c(c4)c(-c4ccccc4)c(-c4ccccc4)c4ccccc45)c3)c3cccc(-c4cccc5ccccc45)c3)c21 ; CC1(c2ccccc2)c2ccccc2-c2ccc(N(c3cccc(-c4ccc5c(c4)c(-c4ccccc4)c(-c4ccccc4)c4ccccc45)c3)c3cccc(-c4cccc5ccccc45)c3)cc21 ; c1ccc(-c2c(-c3ccccc3)c3cc(-c4cccc(N(c5cccc(-c6cccc7ccccc67)c5)c5ccc6c(c5)C(c5ccccc5)(c5ccccc5)c5ccccc5-6)c4)ccc3c3ccccc23)cc1]

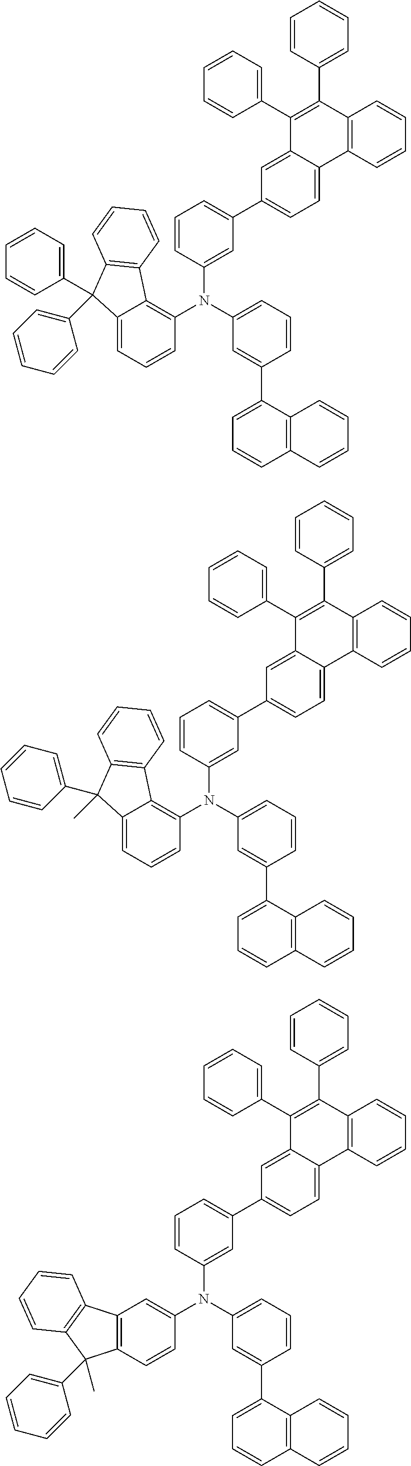

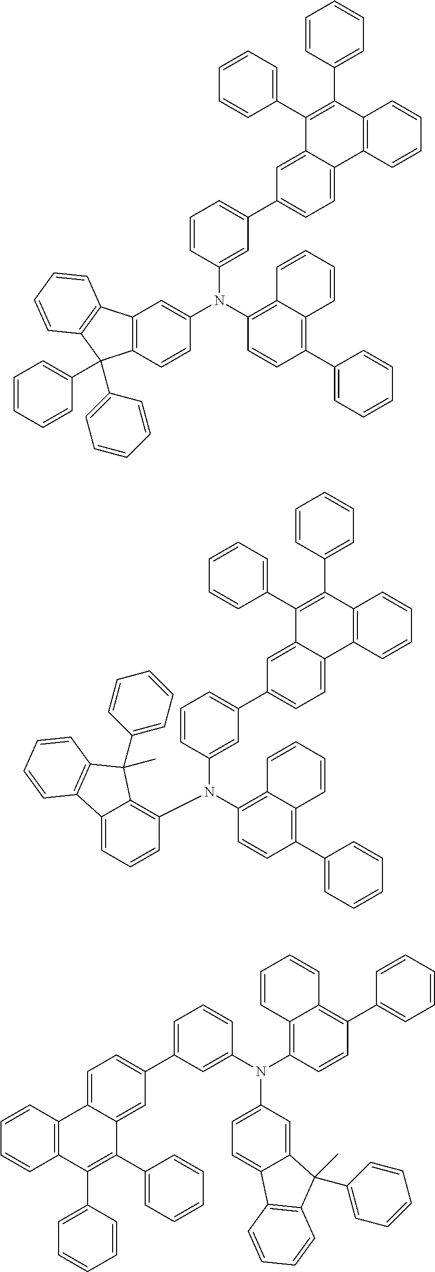 [SMILES: c1ccc(-c2ccc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)c3ccccc23)cc1 ; CC1(c2ccccc2)c2ccccc2-c2cccc(N(c3cccc(-c4ccc5c(c4)c(-c4ccccc4)c(-c4ccccc4)c4ccccc45)c3)c3ccc(-c4ccccc4)c4ccccc34)c21 ; CC1(c2ccccc2)c2ccccc2-c2ccc(N(c3cccc(-c4ccc5c(c4)c(-c4ccccc4)c(-c4ccccc4)c4ccccc45)c3)c3ccc(-c4ccccc4)c4ccccc34)cc21]

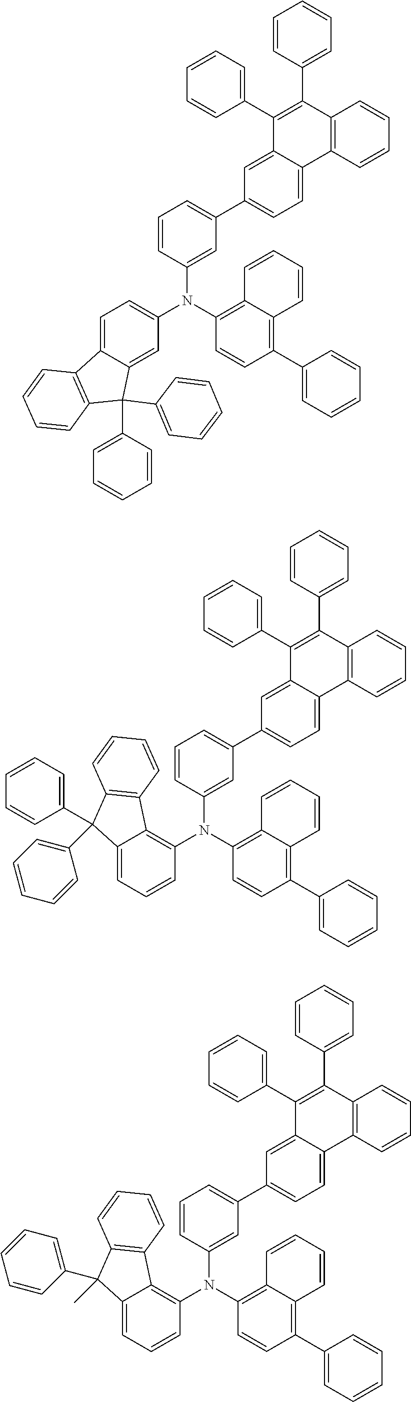

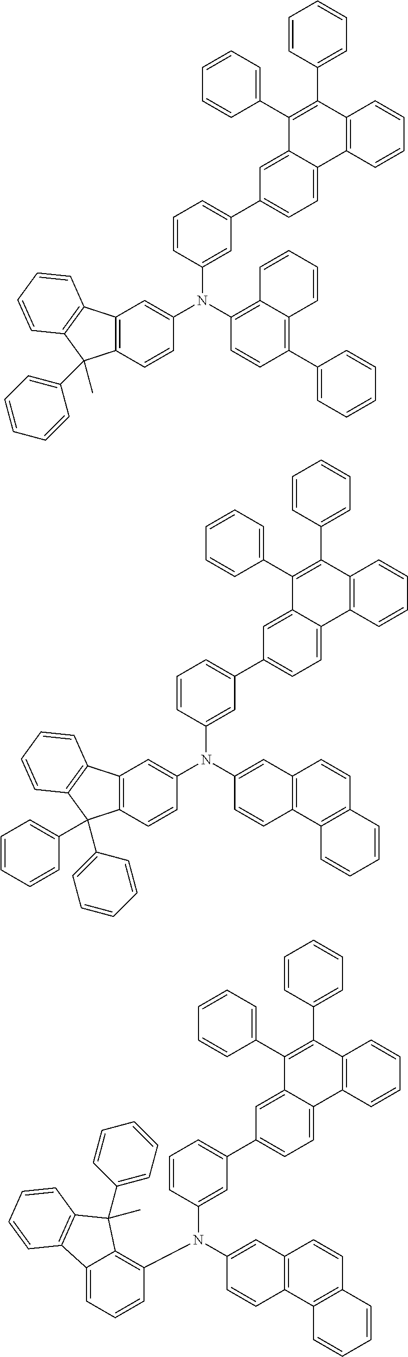 [SMILES: CC1(c2ccccc2)c2ccccc2-c2cc(N(c3cccc(-c4ccc5c(c4)c(-c4ccccc4)c(-c4ccccc4)c4ccccc45)c3)c3ccc(-c4ccccc4)c4ccccc34)ccc21 ; c1ccc(-c2c(-c3ccccc3)c3cc(-c4cccc(N(c5ccc6c(c5)-c5ccccc5C6(c5ccccc5)c5ccccc5)c5ccc6c(ccc7ccccc76)c5)c4)ccc3c3ccccc23)cc1 ; CC1(c2ccccc2)c2ccccc2-c2cccc(N(c3cccc(-c4ccc5c(c4)c(-c4ccccc4)c(-c4ccccc4)c4ccccc45)c3)c3ccc4c(ccc5ccccc54)c3)c21]

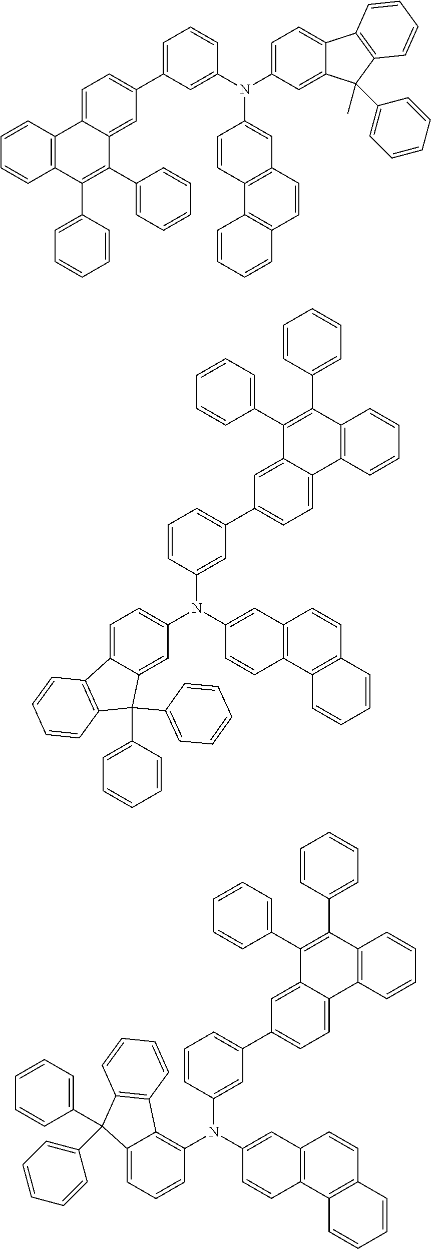 [SMILES: CC1(c2ccccc2)c2ccccc2-c2ccc(N(c3cccc(-c4ccc5c(c4)c(-c4ccccc4)c(-c4ccccc4)c4ccccc45)c3)c3ccc4c(ccc5ccccc54)c3)cc21 ; c1ccc(-c2c(-c3ccccc3)c3cc(-c4cccc(N(c5ccc6c(c5)C(c5ccccc5)(c5ccccc5)c5ccccc5-6)c5ccc6c(ccc7ccccc76)c5)c4)ccc3c3ccccc23)cc1 ; c1ccc(-c2c(-c3ccccc3)c3cc(-c4cccc(N(c5ccc6c(ccc7ccccc76)c5)c5cccc6c5-c5ccccc5C6(c5ccccc5)c5ccccc5)c4)ccc3c3ccccc23)cc1]

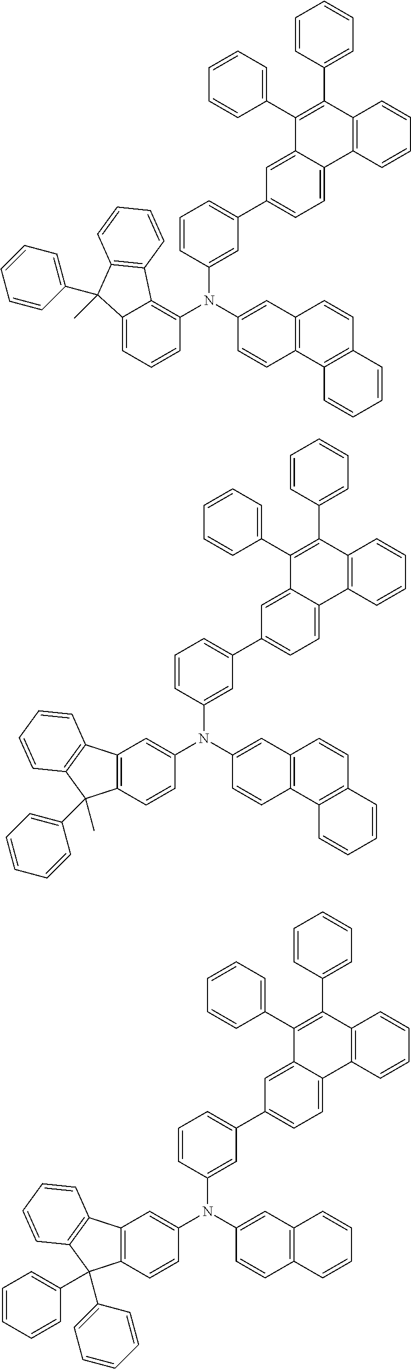

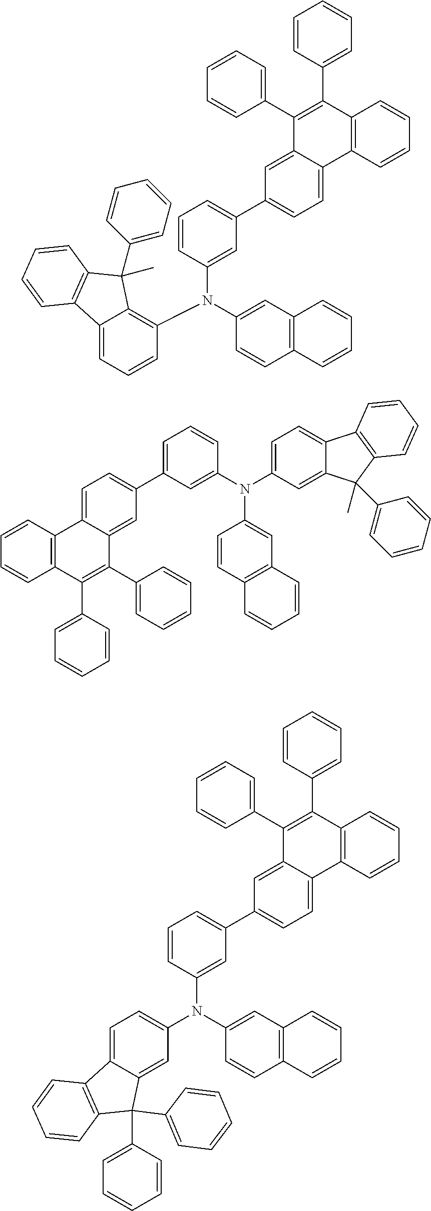 [SMILES: CC1(c2ccccc2)c2ccccc2-c2cccc(N(c3cccc(-c4ccc5c(c4)c(-c4ccccc4)c(-c4ccccc4)c4ccccc45)c3)c3ccc4ccccc4c3)c21 ; CC1(c2ccccc2)c2ccccc2-c2ccc(N(c3cccc(-c4ccc5c(c4)c(-c4ccccc4)c(-c4ccccc4)c4ccccc45)c3)c3ccc4ccccc4c3)cc21 ; c1ccc(-c2c(-c3ccccc3)c3cc(-c4cccc(N(c5ccc6c(c5)C(c5ccccc5)(c5ccccc5)c5ccccc5-6)c5ccc6ccccc6c5)c4)ccc3c3ccccc23)cc1]

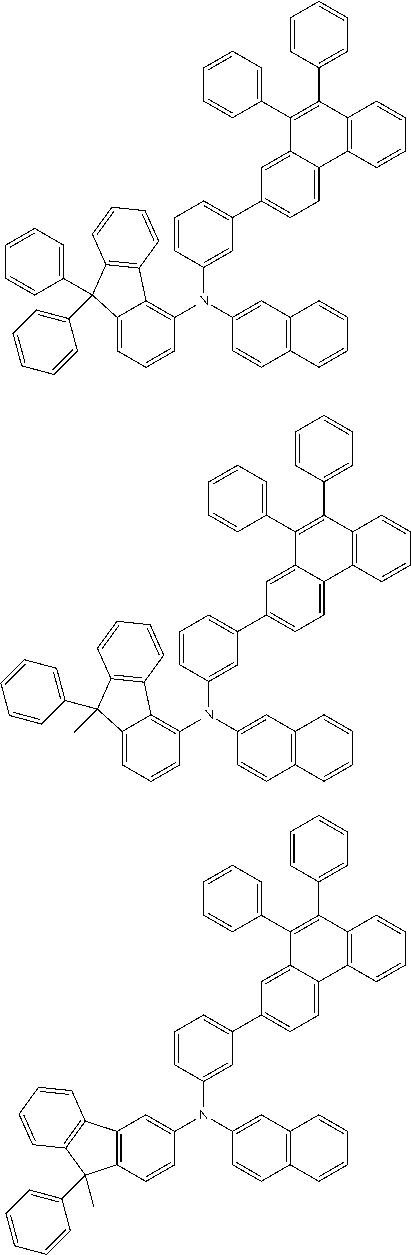

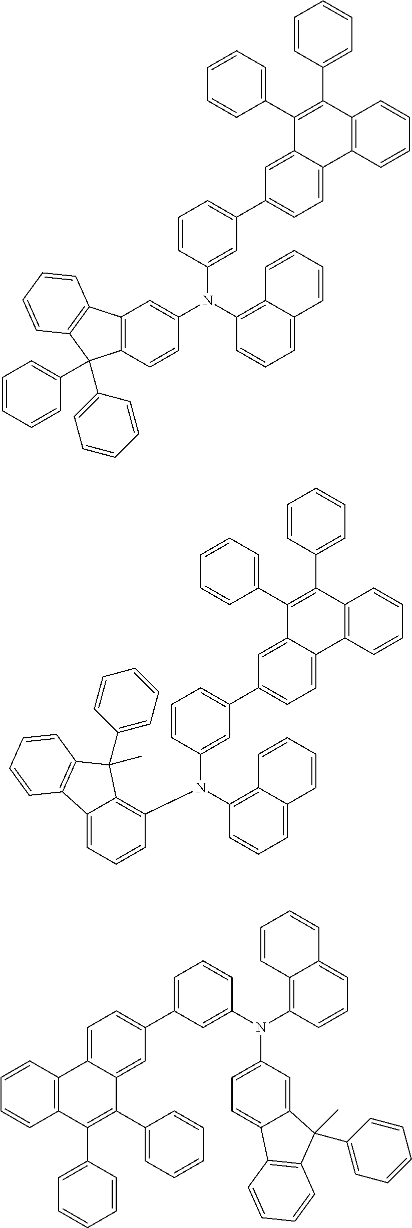 [SMILES: c1ccc(-c2c(-c3ccccc3)c3cc(-c4cccc(N(c5ccc6c(c5)-c5ccccc5C6(c5ccccc5)c5ccccc5)c5cccc6ccccc56)c4)ccc3c3ccccc23)cc1 ; CC1(c2ccccc2)c2ccccc2-c2cccc(N(c3cccc(-c4ccc5c(c4)c(-c4ccccc4)c(-c4ccccc4)c4ccccc45)c3)c3cccc4ccccc34)c21 ; CC1(c2ccccc2)c2ccccc2-c2ccc(N(c3cccc(-c4ccc5c(c4)c(-c4ccccc4)c(-c4ccccc4)c4ccccc45)c3)c3cccc4ccccc34)cc21]

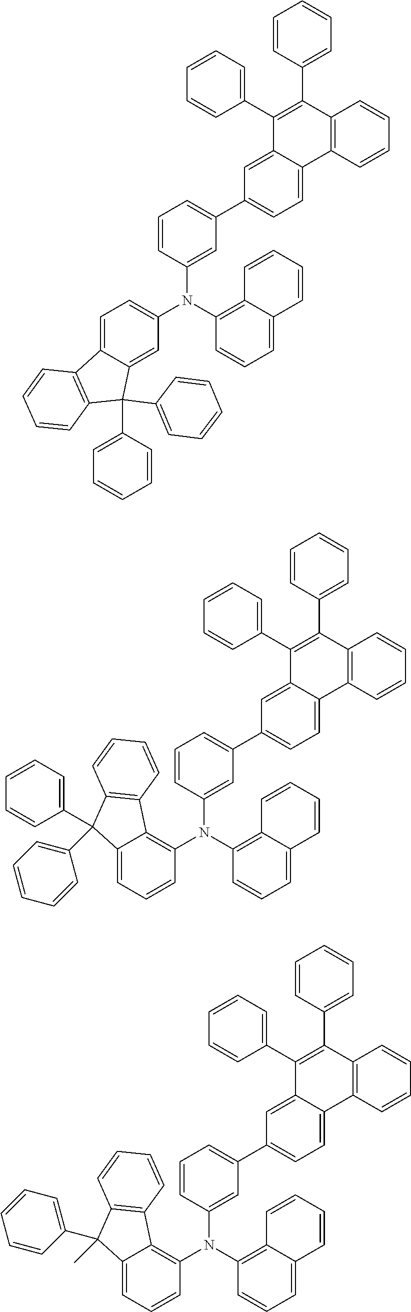 [SMILES: c1ccc(-c2c(-c3ccccc3)c3cc(-c4cccc(N(c5ccc6c(c5)C(c5ccccc5)(c5ccccc5)c5ccccc5-6)c5cccc6ccccc56)c4)ccc3c3ccccc23)cc1 ; c1ccc(-c2c(-c3ccccc3)c3cc(-c4cccc(N(c5cccc6c5-c5ccccc5C6(c5ccccc5)c5ccccc5)c5cccc6ccccc56)c4)ccc3c3ccccc23)cc1 ; CC1(c2ccccc2)c2ccccc2-c2c(N(c3cccc(-c4ccc5c(c4)c(-c4ccccc4)c(-c4ccccc4)c4ccccc45)c3)c3cccc4ccccc34)cccc21]

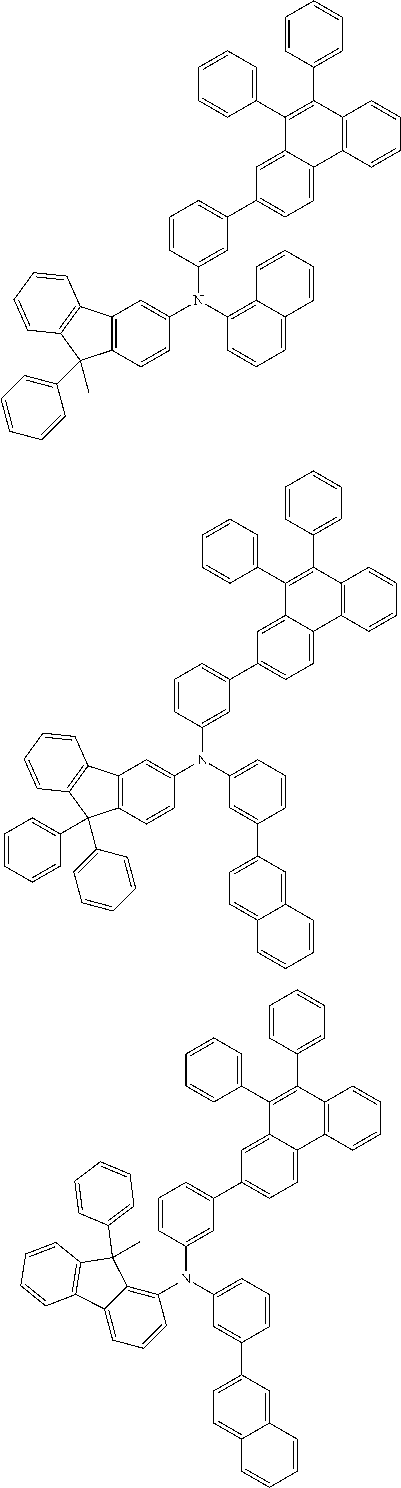

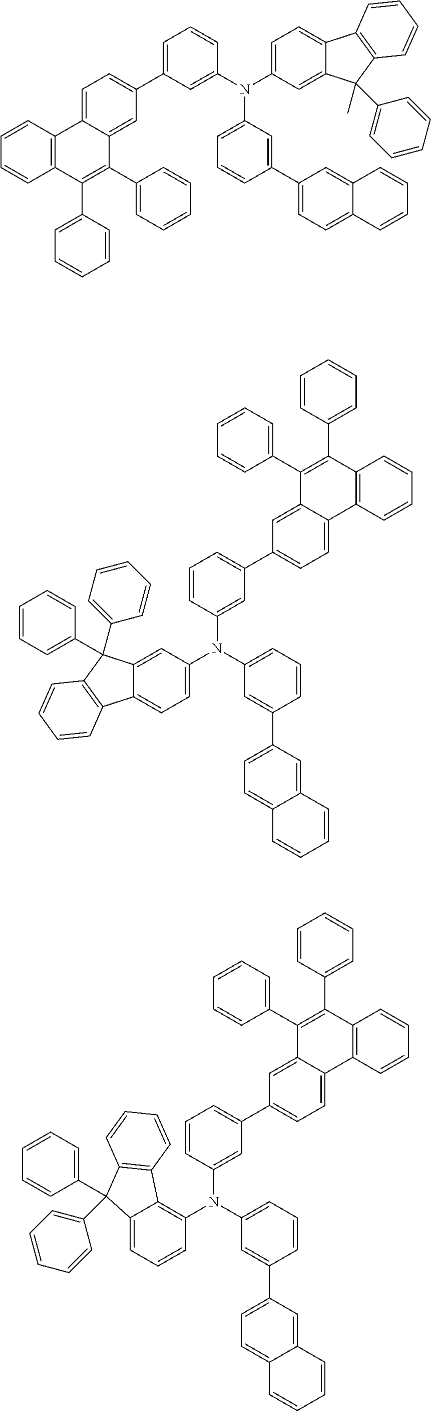 [SMILES: CC1(c2ccccc2)c2ccccc2-c2ccc(N(c3cccc(-c4ccc5ccccc5c4)c3)c3cccc(-c4ccc5c(c4)c(-c4ccccc4)c(-c4ccccc4)c4ccccc45)c3)cc21 ; c1ccc(-c2c(-c3ccccc3)c3cc(-c4cccc(N(c5cccc(-c6ccc7ccccc7c6)c5)c5ccc6c(c5)C(c5ccccc5)(c5ccccc5)c5ccccc5-6)c4)ccc3c3ccccc23)cc1 ; c1ccc(-c2c(-c3ccccc3)c3cc(-c4cccc(N(c5cccc(-c6ccc7ccccc7c6)c5)c5cccc6c5-c5ccccc5C6(c5ccccc5)c5ccccc5)c4)ccc3c3ccccc23)cc1]

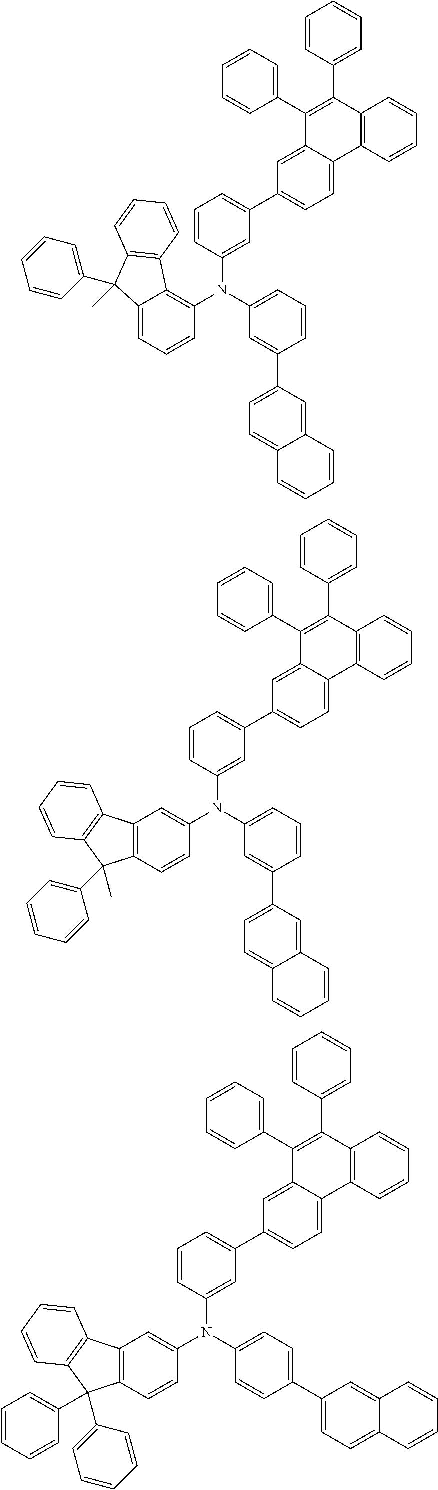

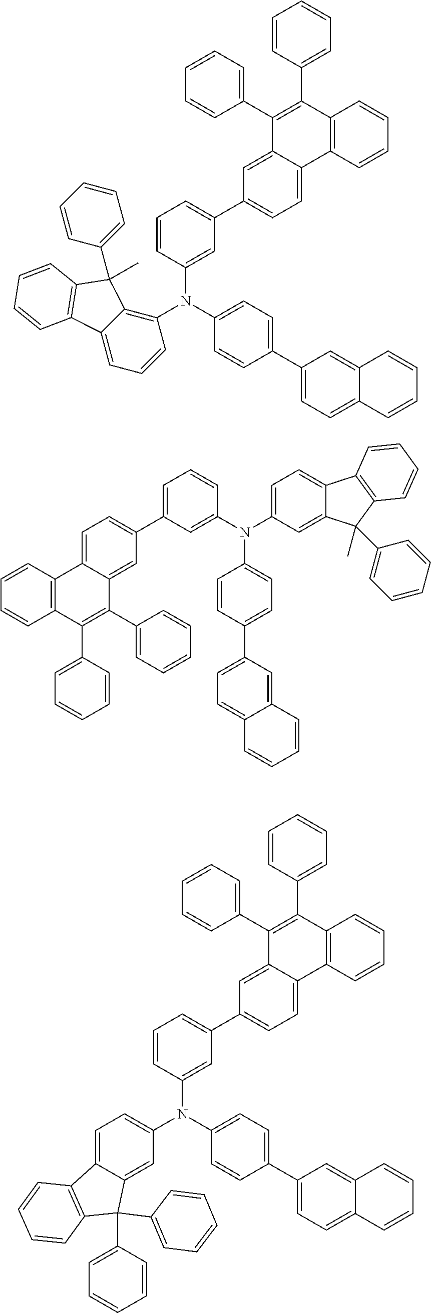

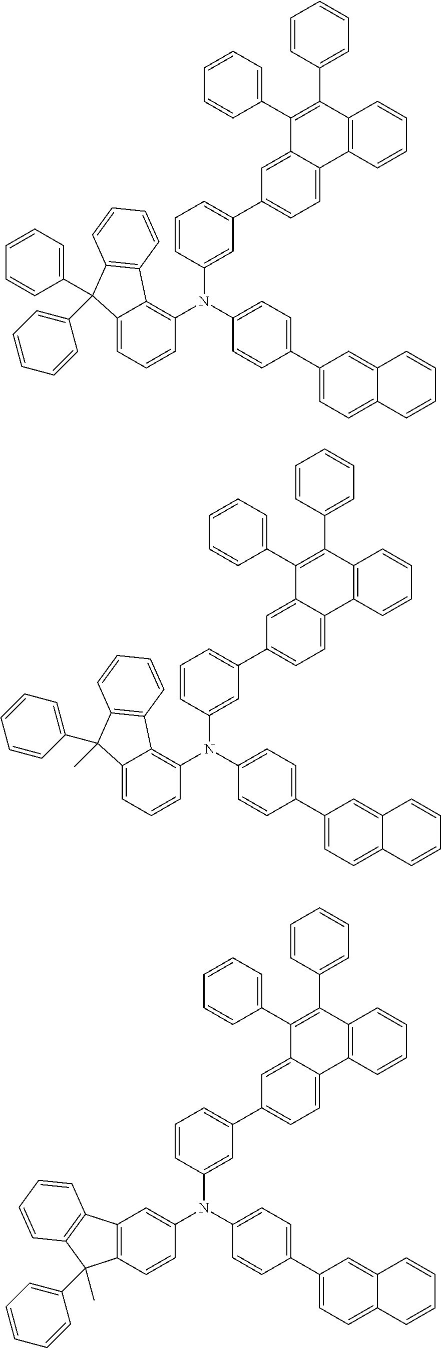

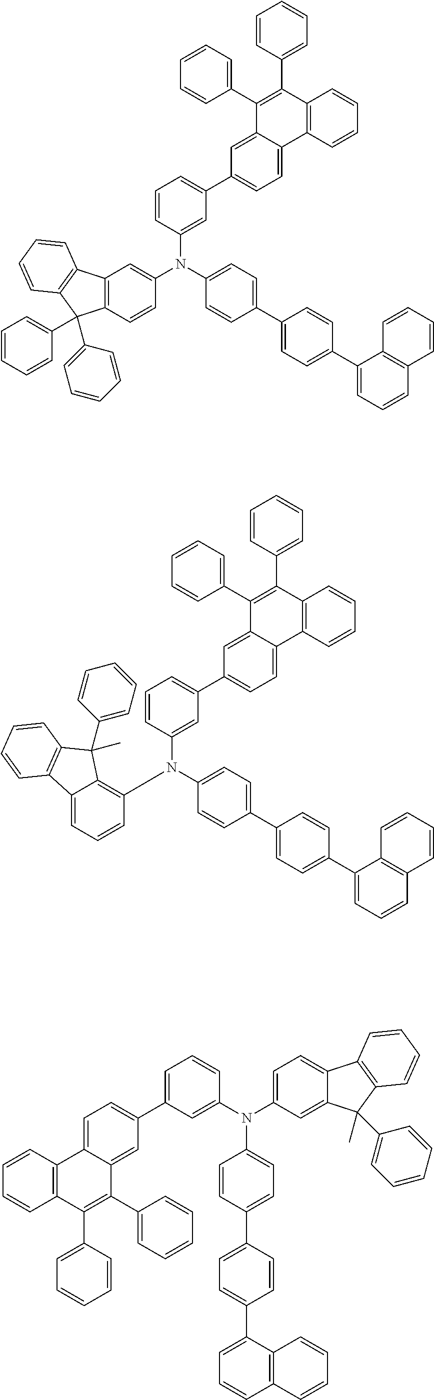 [SMILES: c1ccc(-c2c(-c3ccccc3)c3cc(-c4cccc(N(c5ccc(-c6ccc(-c7cccc8ccccc78)cc6)cc5)c5ccc6c(c5)-c5ccccc5C6(c5ccccc5)c5ccccc5)c4)ccc3c3ccccc23)cc1 ; CC1(c2ccccc2)c2ccccc2-c2cccc(N(c3ccc(-c4ccc(-c5cccc6ccccc56)cc4)cc3)c3cccc(-c4ccc5c(c4)c(-c4ccccc4)c(-c4ccccc4)c4ccccc45)c3)c21 ; CC1(c2ccccc2)c2ccccc2-c2ccc(N(c3ccc(-c4ccc(-c5cccc6ccccc56)cc4)cc3)c3cccc(-c4ccc5c(c4)c(-c4ccccc4)c(-c4ccccc4)c4ccccc45)c3)cc21]

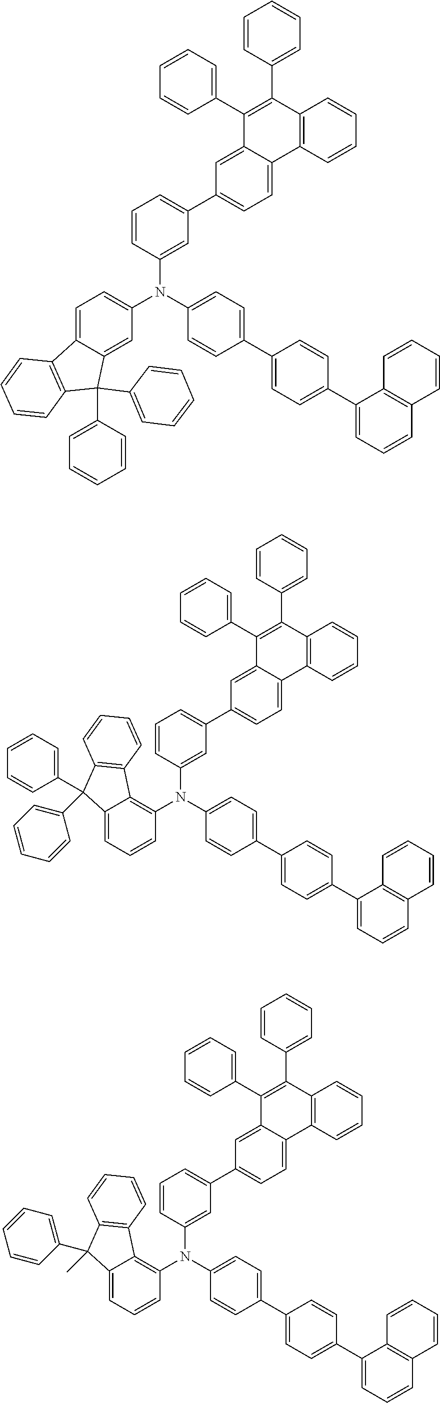 [SMILES: c1ccc(-c2c(-c3ccccc3)c3cc(-c4cccc(N(c5ccc(-c6ccc(-c7cccc8ccccc78)cc6)cc5)c5ccc6c(c5)C(c5ccccc5)(c5ccccc5)c5ccccc5-6)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5cccc6c5-c5ccccc5C6(c5ccccc5)c5ccccc5)c4)ccc3c3ccccc23)cc1 ; CC1(c2ccccc2)c2ccccc2-c2c(N(c3ccc(-c4ccc(-c5cccc6ccccc56)cc4)cc3)c3cccc(-c4ccc5c(c4)c(-c4ccccc4)c(-c4ccccc4)c4ccccc45)c3)cccc21]

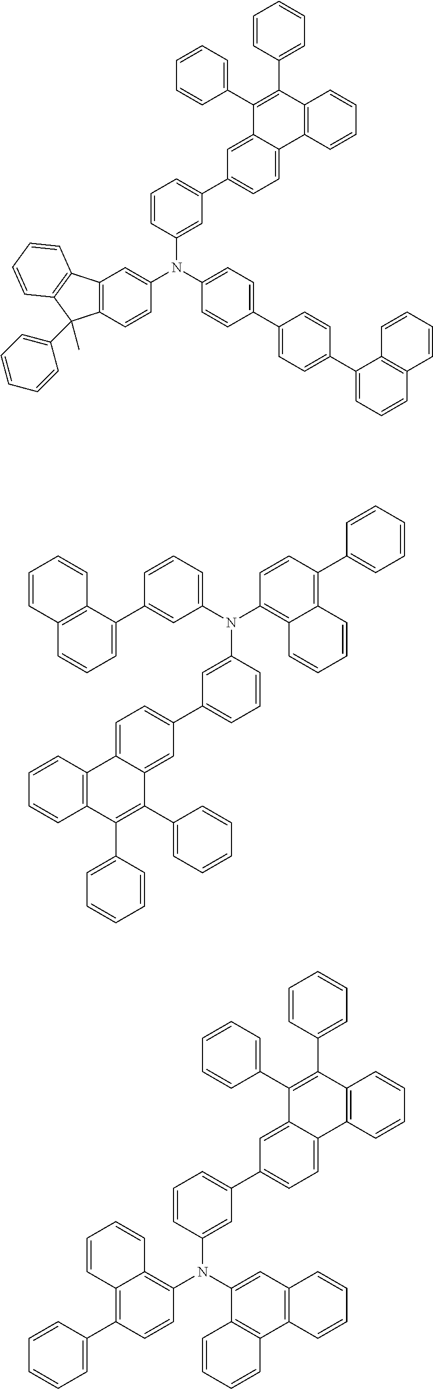 [SMILES: CC1(c2ccccc2)c2ccccc2-c2cc(N(c3ccc(-c4ccc(-c5cccc6ccccc56)cc4)cc3)c3cccc(-c4ccc5c(c4)c(-c4ccccc4)c(-c4ccccc4)c4ccccc45)c3)ccc21 ; c1ccc(-c2ccc(N(c3cccc(-c4ccc5c(c4)c(-c4ccccc4)c(-c4ccccc4)c4ccccc45)c3)c3cccc(-c4cccc5ccccc45)c3)c3ccccc23)cc1 ; c1ccc(-c2ccc(N(c3cccc(-c4ccc5c(c4)c(-c4ccccc4)c(-c4ccccc4)c4ccccc45)c3)c3cc4ccccc4c4ccccc34)c3ccccc23)cc1]

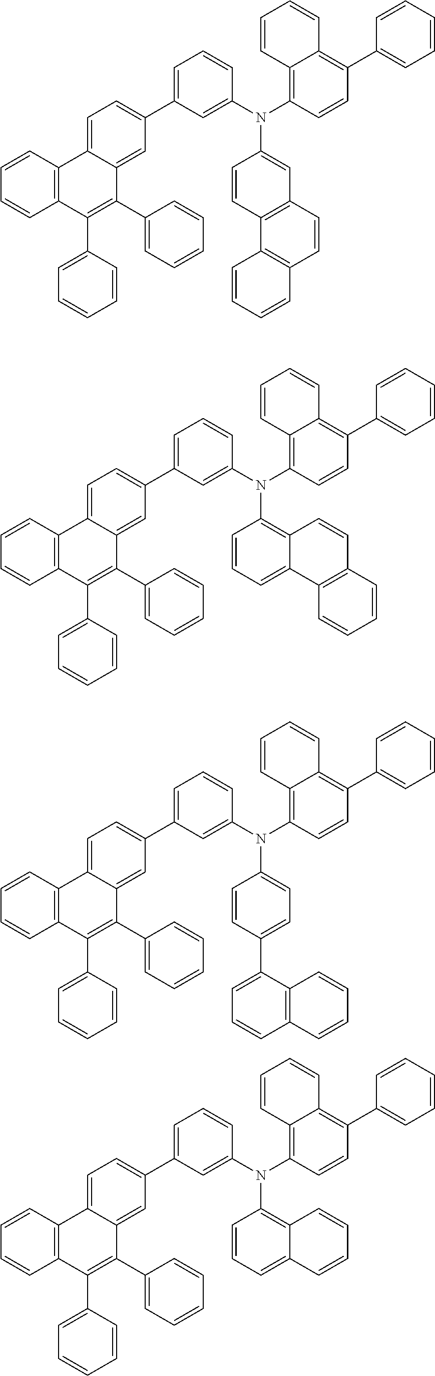

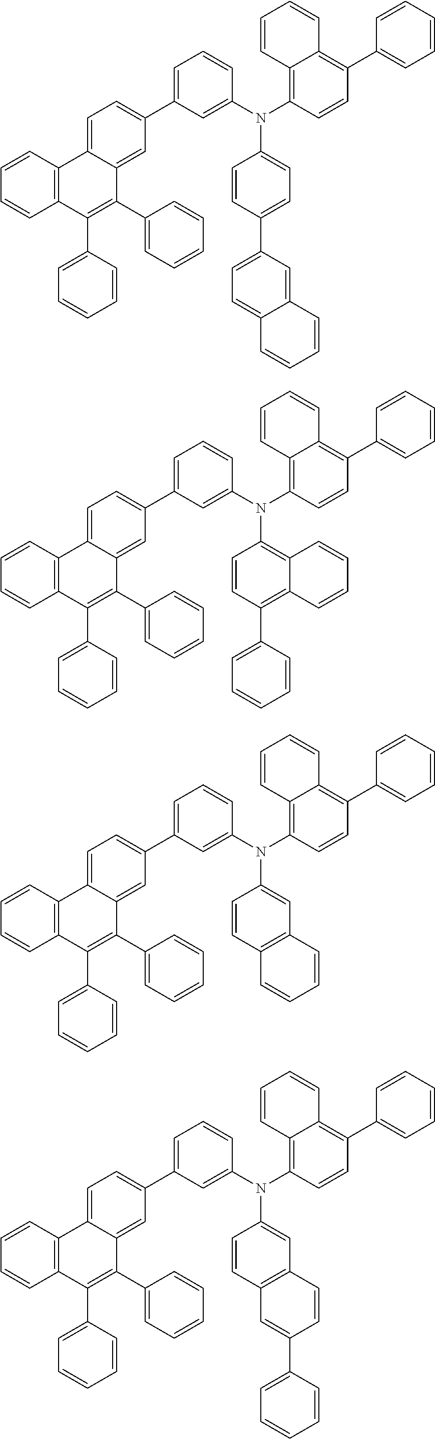

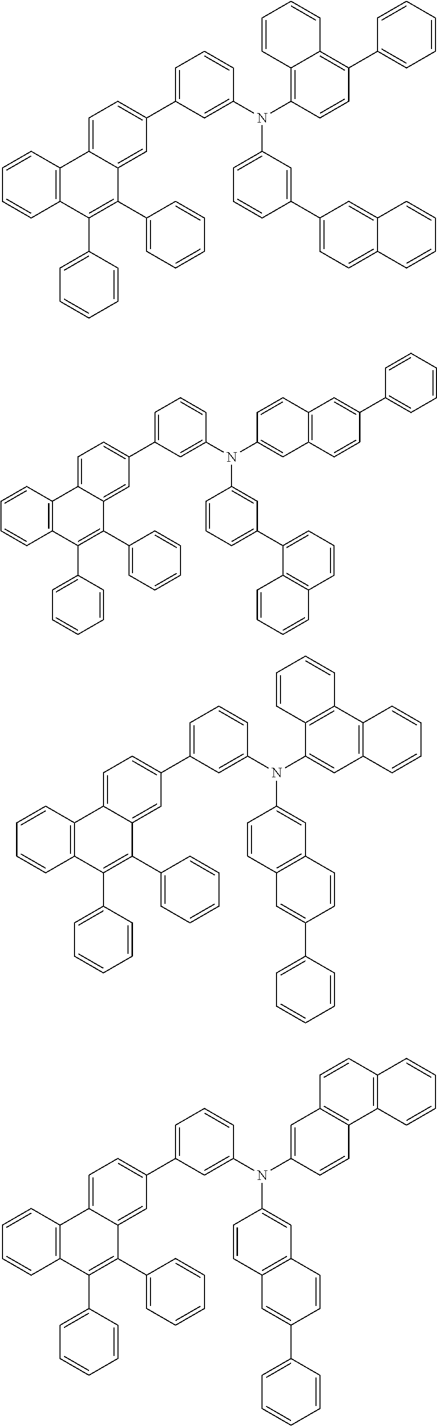

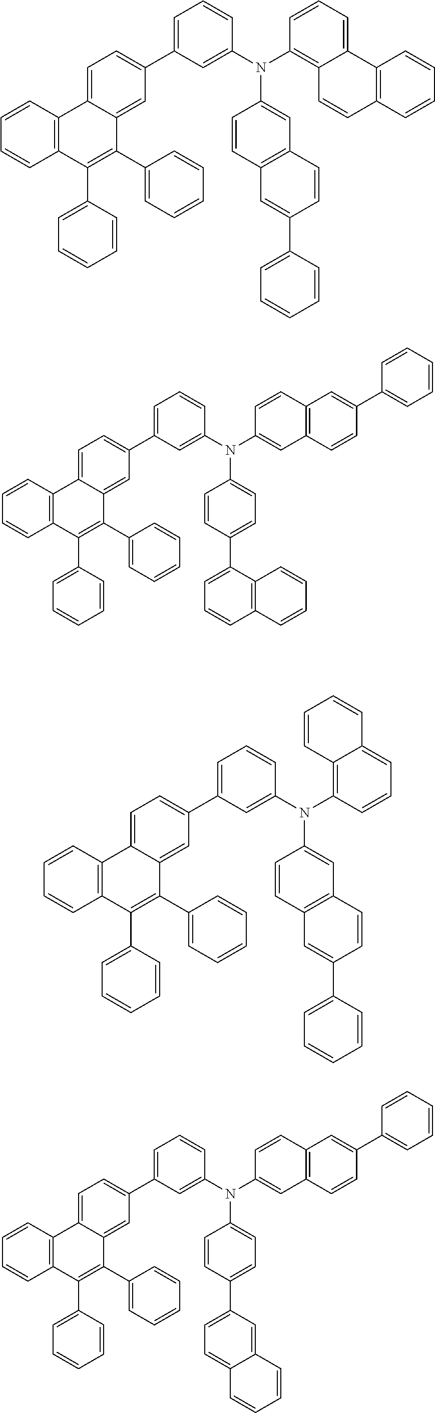 [SMILES: c1ccc(-c2ccc3cc(N(c4cccc(-c5ccc6c(c5)c(-c5ccccc5)c(-c5ccccc5)c5ccccc56)c4)c4cccc5c4ccc4ccccc45)ccc3c2)cc1 ; c1ccc(-c2ccc3cc(N(c4ccc(-c5cccc6ccccc56)cc4)c4cccc(-c5ccc6c(c5)c(-c5ccccc5)c(-c5ccccc5)c5ccccc56)c4)ccc3c2)cc1 ; c1ccc(-c2ccc3cc(N(c4cccc(-c5ccc6c(c5)c(-c5ccccc5)c(-c5ccccc5)c5ccccc56)c4)c4cccc5ccccc45)ccc3c2)cc1 ; c1ccc(-c2ccc3cc(N(c4ccc(-c5ccc6ccccc6c5)cc4)c4cccc(-c5ccc6c(c5)c(-c5ccccc5)c(-c5ccccc5)c5ccccc56)c4)ccc3c2)cc1]

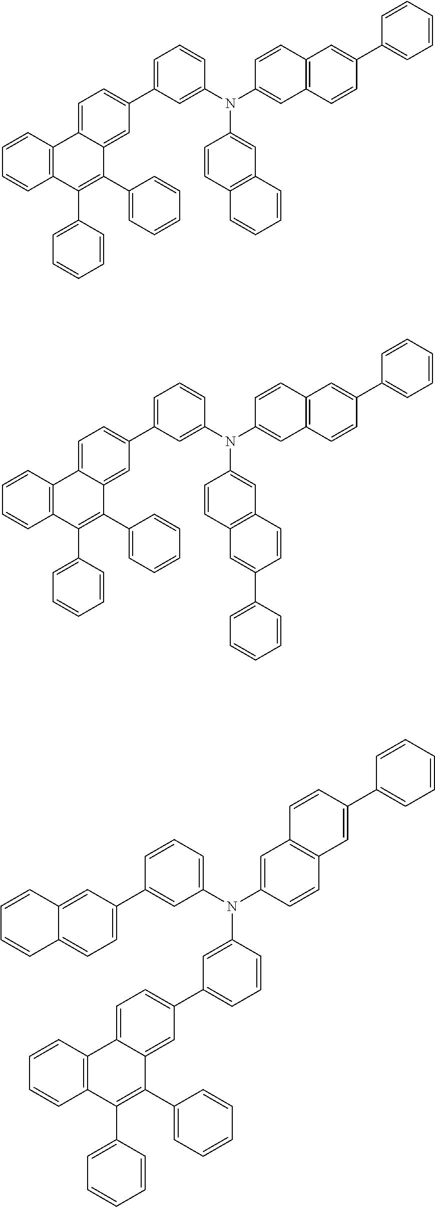 [SMILES: c1ccc(-c2ccc3cc(N(c4cccc(-c5ccc6c(c5)c(-c5ccccc5)c(-c5ccccc5)c5ccccc56)c4)c4ccc5ccccc5c4)ccc3c2)cc1 ; c1ccc(-c2ccc3cc(N(c4cccc(-c5ccc6c(c5)c(-c5ccccc5)c(-c5ccccc5)c5ccccc56)c4)c4ccc5cc(-c6ccccc6)ccc5c4)ccc3c2)cc1 ; c1ccc(-c2ccc3cc(N(c4cccc(-c5ccc6ccccc6c5)c4)c4cccc(-c5ccc6c(c5)c(-c5ccccc5)c(-c5ccccc5)c5ccccc56)c4)ccc3c2)cc1]

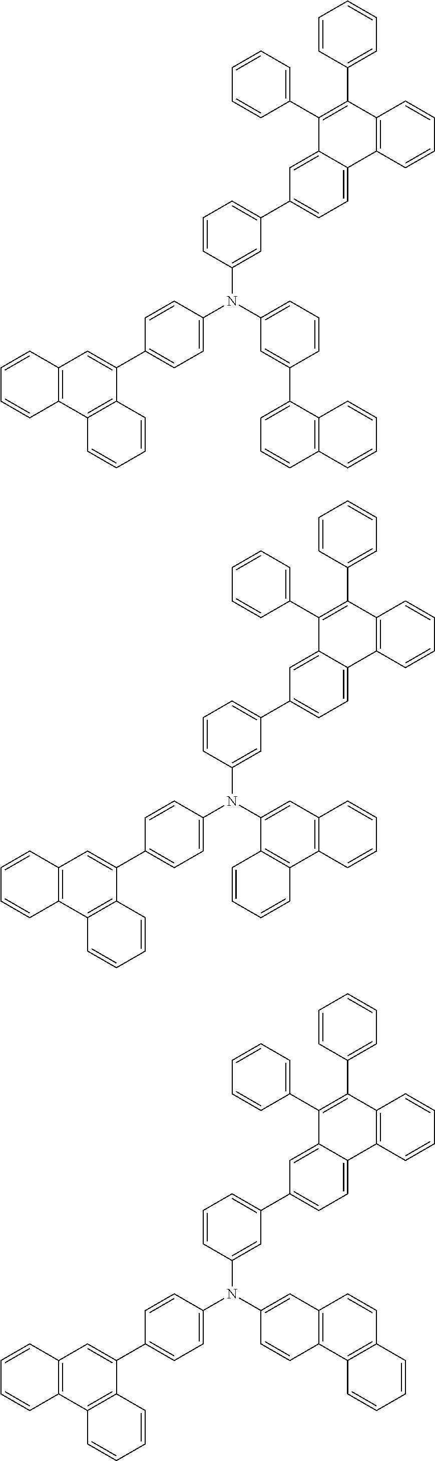

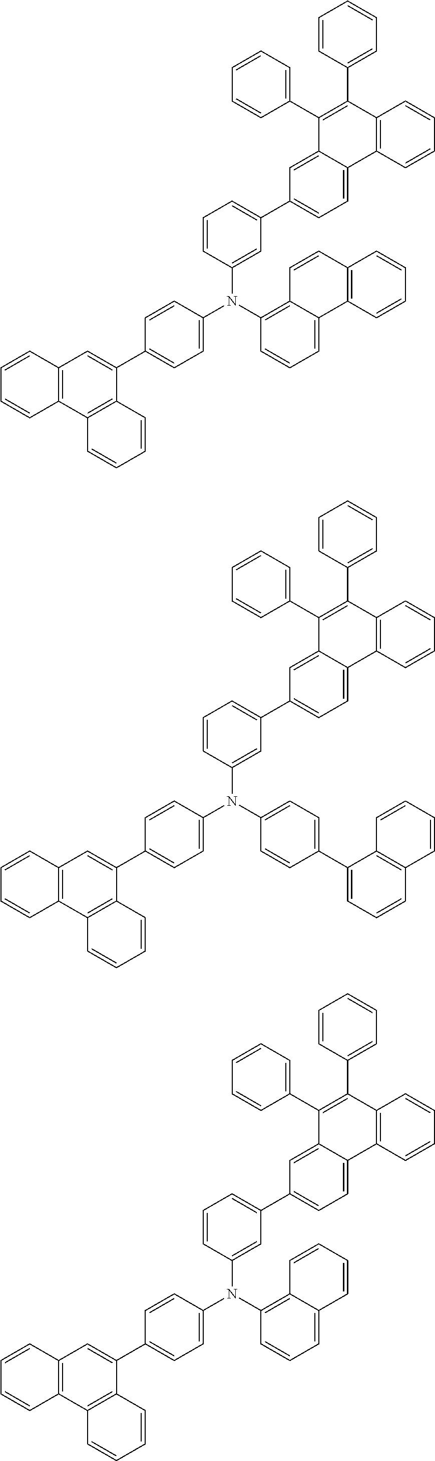 [SMILES: c1ccc(-c2c(-c3ccccc3)c3cc(-c4cccc(N(c5ccc(-c6cc7ccccc7c7ccccc67)cc5)c5cccc6c5ccc5ccccc56)c4)ccc3c3ccccc23)cc1 ; c1ccc(-c2c(-c3ccccc3)c3cc(-c4cccc(N(c5ccc(-c6cccc7ccccc67)cc5)c5ccc(-c6cc7ccccc7c7ccccc67)cc5)c4)ccc3c3ccccc23)cc1 ; c1ccc(-c2c(-c3ccccc3)c3cc(-c4cccc(N(c5ccc(-c6cc7ccccc7c7ccccc67)cc5)c5cccc6ccccc56)c4)ccc3c3ccccc23)cc1]

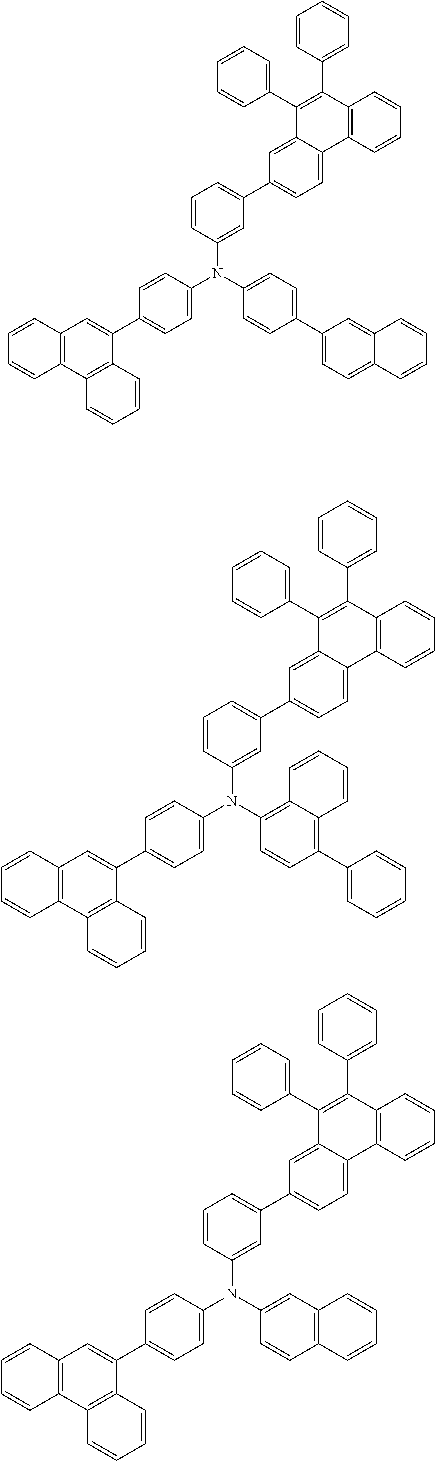 [SMILES: c1ccc(-c2c(-c3ccccc3)c3cc(-c4cccc(N(c5ccc(-c6ccc7ccccc7c6)cc5)c5ccc(-c6cc7ccccc7c7ccccc67)cc5)c4)ccc3c3ccccc23)cc1 ; c1ccc(-c2ccc(N(c3ccc(-c4cc5ccccc5c5ccccc45)cc3)c3cccc(-c4ccc5c(c4)c(-c4ccccc4)c(-c4ccccc4)c4ccccc45)c3)c3ccccc23)cc1 ; c1ccc(-c2c(-c3ccccc3)c3cc(-c4cccc(N(c5ccc(-c6cc7ccccc7c7ccccc67)cc5)c5ccc6ccccc6c5)c4)ccc3c3ccccc23)cc1]

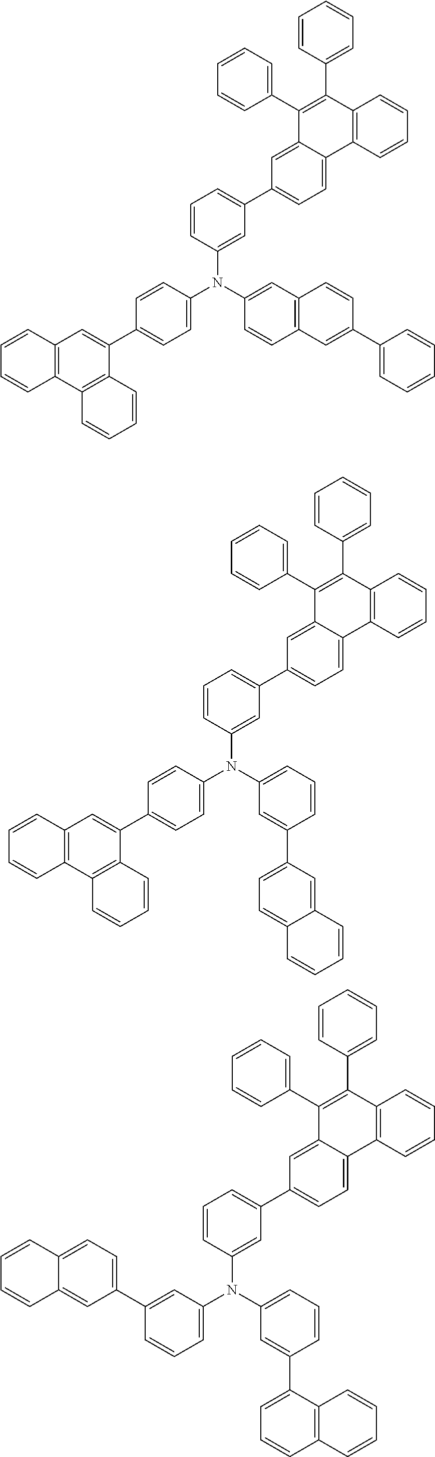

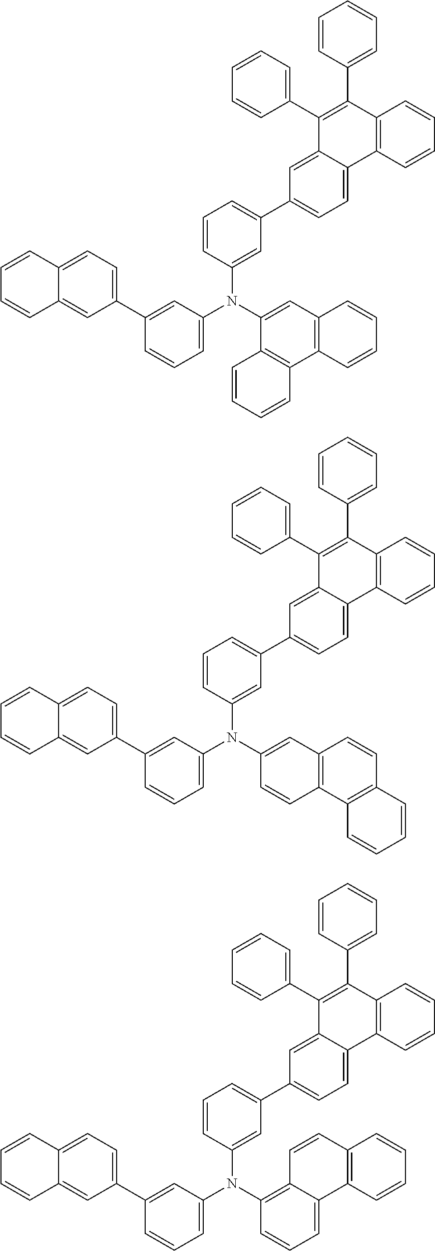

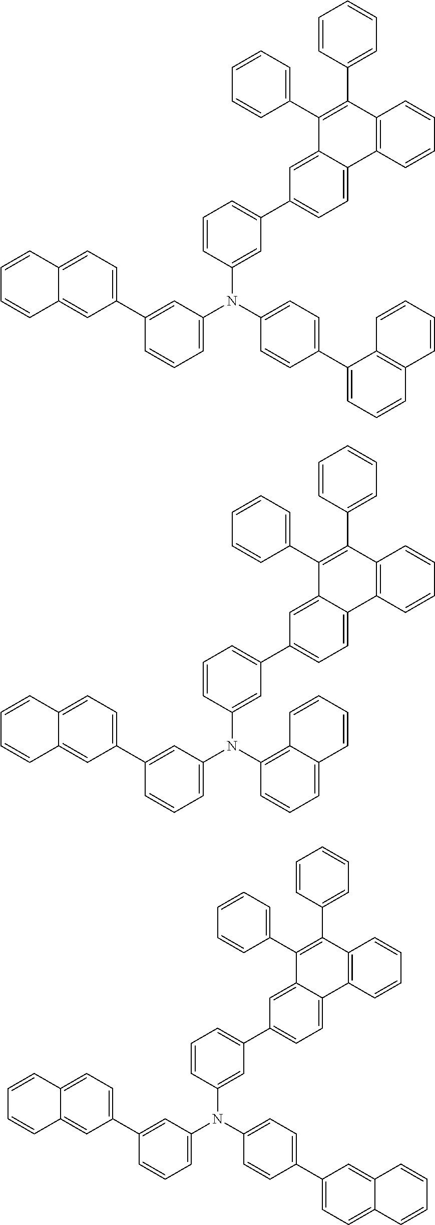

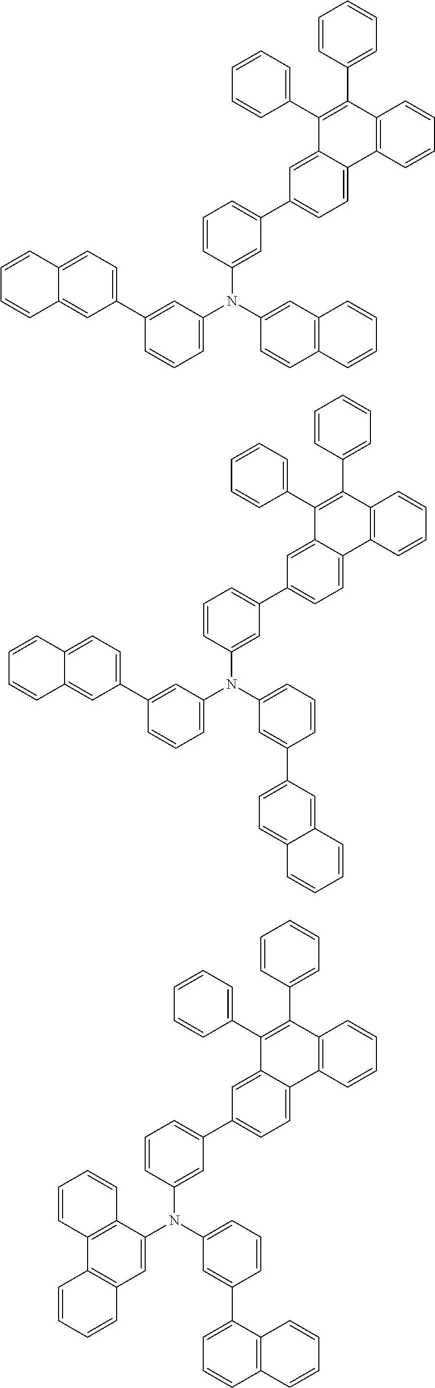

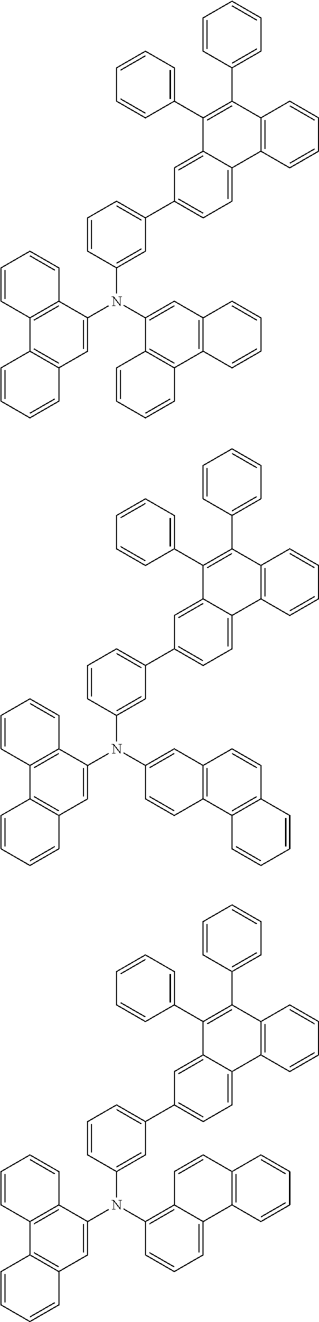 [SMILES: c1ccc(-c2c(-c3ccccc3)c3cc(-c4cccc(N(c5cc6ccccc6c6ccccc56)c5cc6ccccc6c6ccccc56)c4)ccc3c3ccccc23)cc1 ; c1ccc(-c2c(-c3ccccc3)c3cc(-c4cccc(N(c5ccc6c(ccc7ccccc76)c5)c5cc6ccccc6c6ccccc56)c4)ccc3c3ccccc23)cc1 ; c1ccc(-c2c(-c3ccccc3)c3cc(-c4cccc(N(c5cccc6c5ccc5ccccc56)c5cc6ccccc6c6ccccc56)c4)ccc3c3ccccc23)cc1]

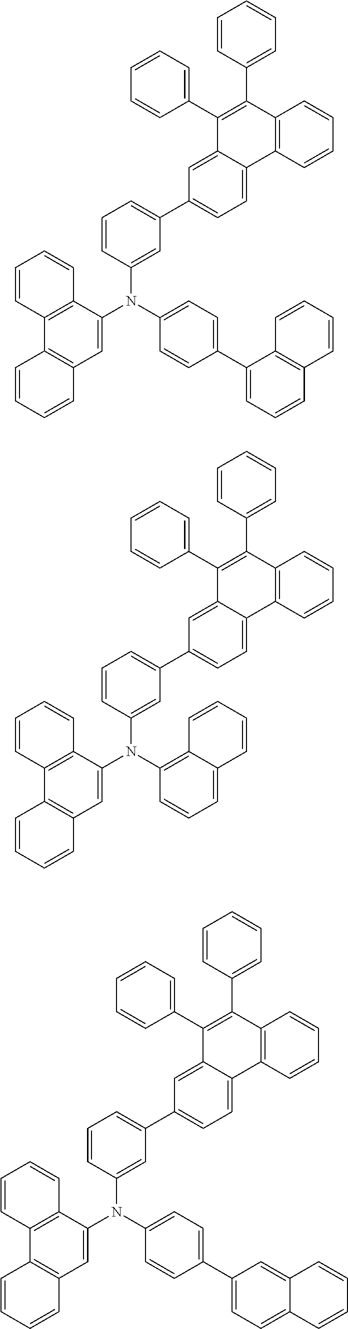

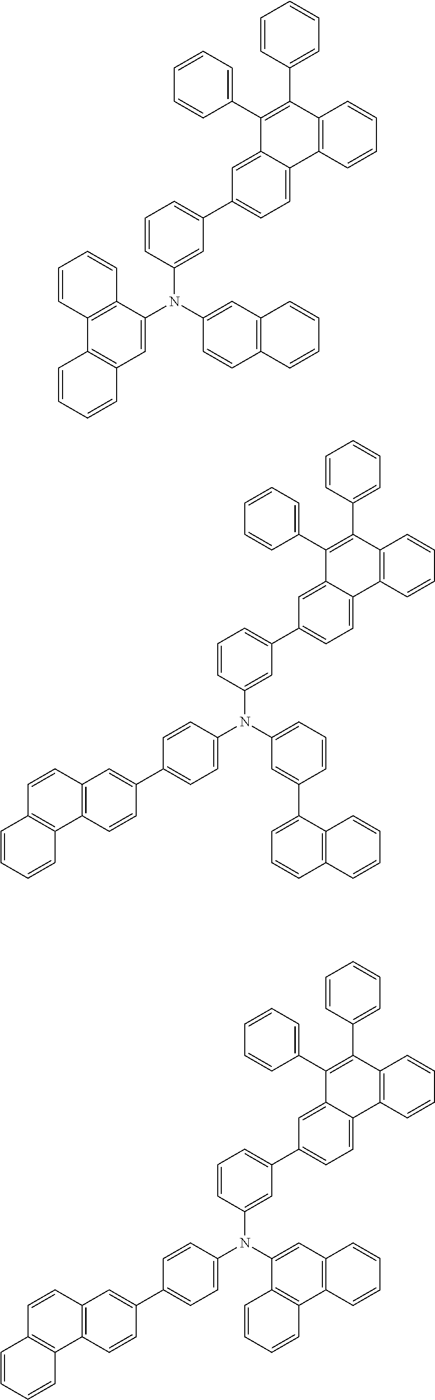 [SMILES: c1ccc(-c2c(-c3ccccc3)c3cc(-c4cccc(N(c5ccc6ccccc6c5)c5cc6ccccc6c6ccccc56)c4)ccc3c3ccccc23)cc1 ; c1ccc(-c2c(-c3ccccc3)c3cc(-c4cccc(N(c5ccc(-c6ccc7c(ccc8ccccc87)c6)cc5)c5cccc(-c6cccc7ccccc67)c5)c4)ccc3c3ccccc23)cc1 ; c1ccc(-c2c(-c3ccccc3)c3cc(-c4cccc(N(c5ccc(-c6ccc7c(ccc8ccccc87)c6)cc5)c5cc6ccccc6c6ccccc56)c4)ccc3c3ccccc23)cc1]

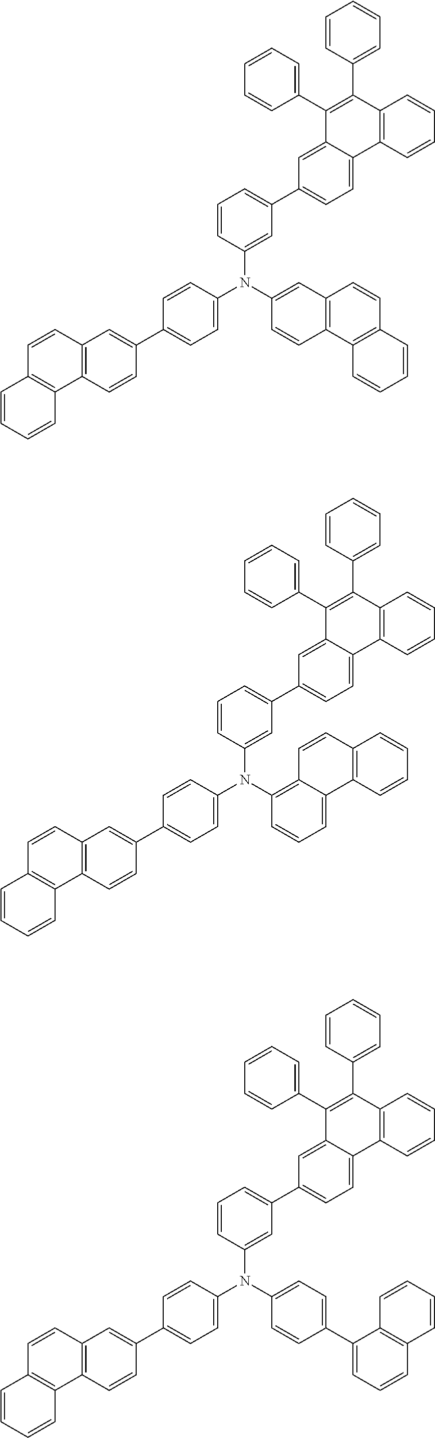 [SMILES: c1ccc(-c2c(-c3ccccc3)c3cc(-c4cccc(N(c5ccc(-c6ccc7c(ccc8ccccc87)c6)cc5)c5ccc6c(ccc7ccccc76)c5)c4)ccc3c3ccccc23)cc1 ; c1ccc(-c2c(-c3ccccc3)c3cc(-c4cccc(N(c5ccc(-c6ccc7c(ccc8ccccc87)c6)cc5)c5cccc6c5ccc5ccccc56)c4)ccc3c3ccccc23)cc1 ; c1ccc(-c2c(-c3ccccc3)c3cc(-c4cccc(N(c5ccc(-c6ccc7c(ccc8ccccc87)c6)cc5)c5ccc(-c6cccc7ccccc67)cc5)c4)ccc3c3ccccc23)cc1]

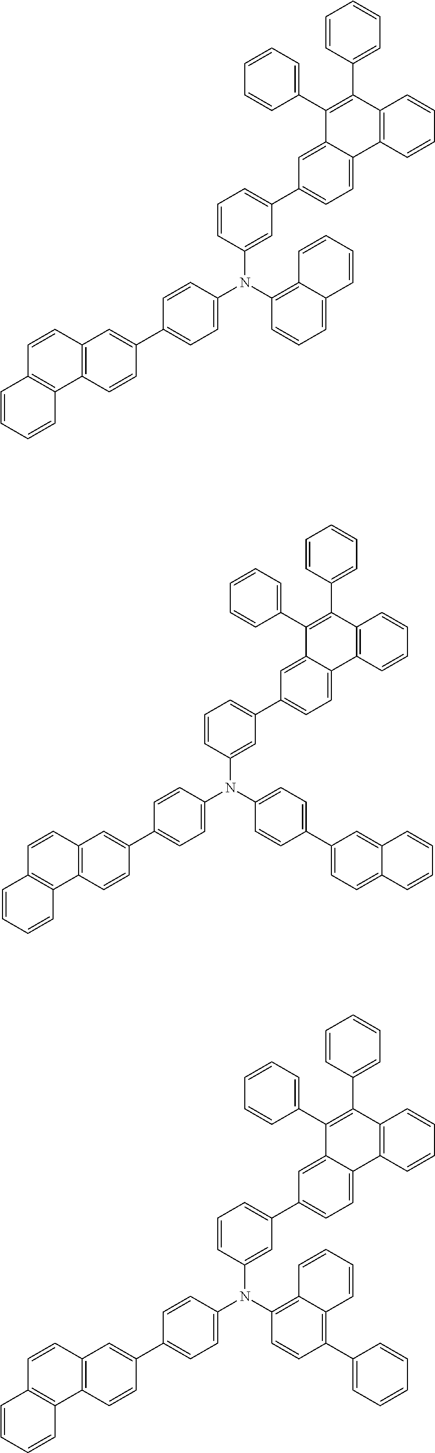 [SMILES: c1ccc(-c2c(-c3ccccc3)c3cc(-c4cccc(N(c5ccc(-c6ccc7c(ccc8ccccc87)c6)cc5)c5cccc6ccccc56)c4)ccc3c3ccccc23)cc1 ; c1ccc(-c2c(-c3ccccc3)c3cc(-c4cccc(N(c5ccc(-c6ccc7ccccc7c6)cc5)c5ccc(-c6ccc7c(ccc8ccccc87)c6)cc5)c4)ccc3c3ccccc23)cc1 ; c1ccc(-c2ccc(N(c3ccc(-c4ccc5c(ccc6ccccc65)c4)cc3)c3cccc(-c4ccc5c(c4)c(-c4ccccc4)c(-c4ccccc4)c4ccccc45)c3)c3ccccc23)cc1]

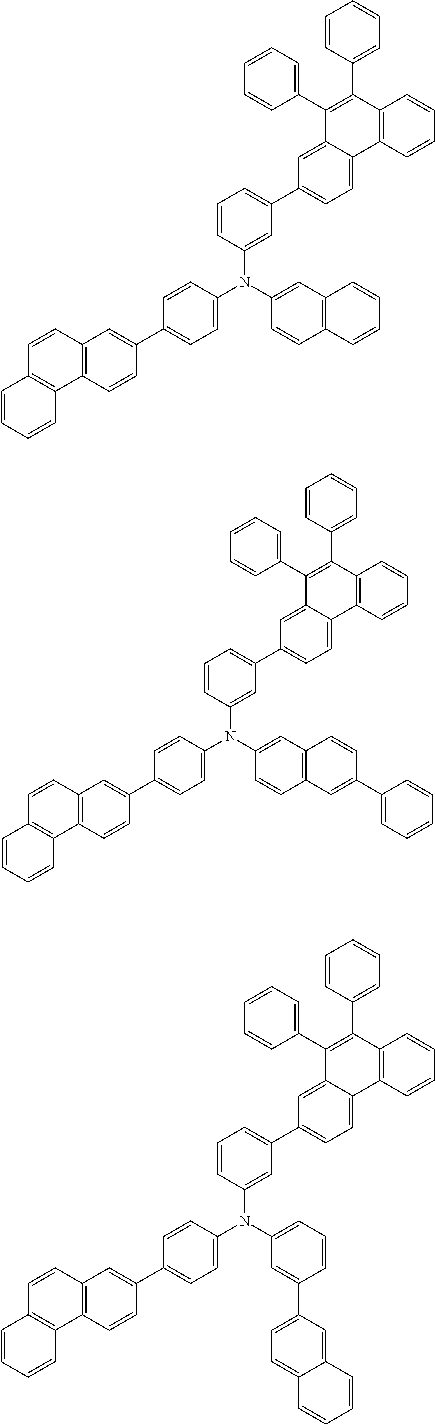 [SMILES: c1ccc(-c2c(-c3ccccc3)c3cc(-c4cccc(N(c5ccc(-c6ccc7c(ccc8ccccc87)c6)cc5)c5ccc6ccccc6c5)c4)ccc3c3ccccc23)cc1 ; c1ccc(-c2ccc3cc(N(c4ccc(-c5ccc6c(ccc7ccccc76)c5)cc4)c4cccc(-c5ccc6c(c5)c(-c5ccccc5)c(-c5ccccc5)c5ccccc56)c4)ccc3c2)cc1 ; c1ccc(-c2c(-c3ccccc3)c3cc(-c4cccc(N(c5ccc(-c6ccc7c(ccc8ccccc87)c6)cc5)c5cccc(-c6ccc7ccccc7c6)c5)c4)ccc3c3ccccc23)cc1]

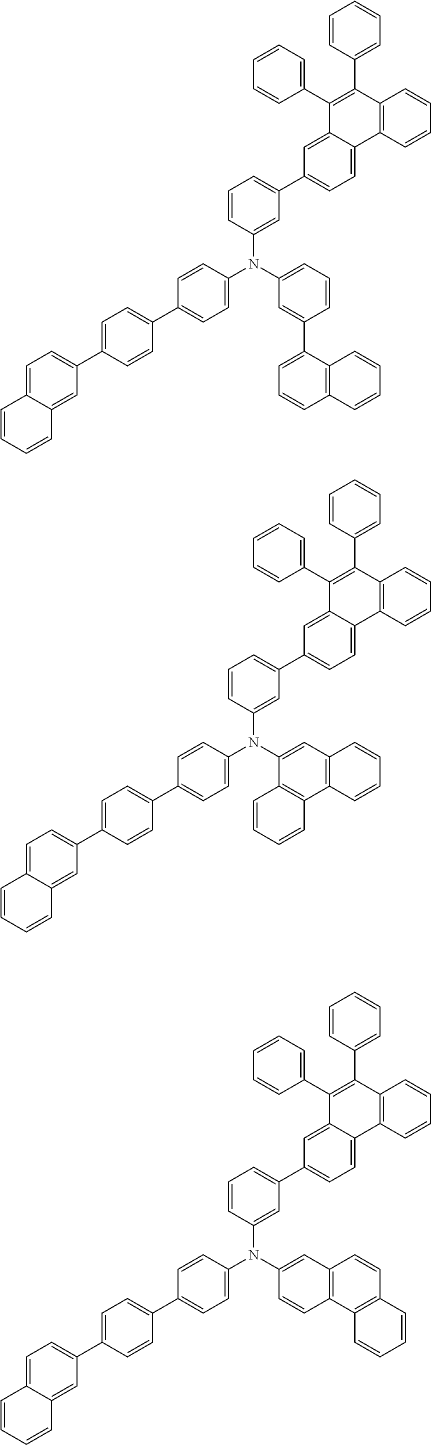 [SMILES: c1ccc(-c2c(-c3ccccc3)c3cc(-c4cccc(N(c5ccc(-c6ccc(-c7ccc8ccccc8c7)cc6)cc5)c5cccc(-c6cccc7ccccc67)c5)c4)ccc3c3ccccc23)cc1 ; c1ccc(-c2c(-c3ccccc3)c3cc(-c4cccc(N(c5ccc(-c6ccc(-c7ccc8ccccc8c7)cc6)cc5)c5cc6ccccc6c6ccccc56)c4)ccc3c3ccccc23)cc1 ; c1ccc(-c2c(-c3ccccc3)c3cc(-c4cccc(N(c5ccc(-c6ccc(-c7ccc8ccccc8c7)cc6)cc5)c5ccc6c(ccc7ccccc76)c5)c4)ccc3c3ccccc23)cc1]

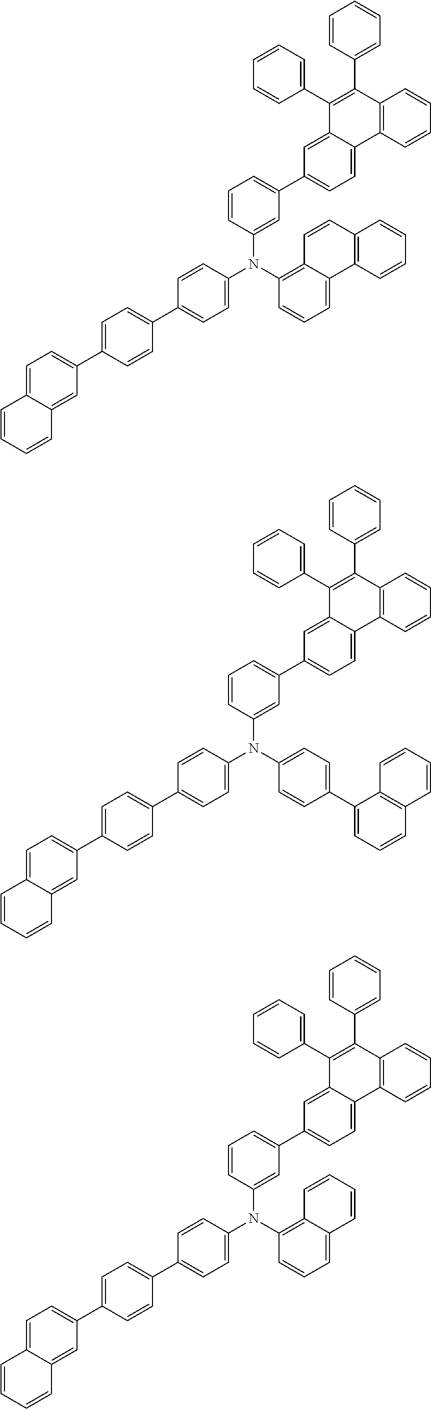 [SMILES: c1ccc(-c2c(-c3ccccc3)c3cc(-c4cccc(N(c5ccc(-c6ccc(-c7ccc8ccccc8c7)cc6)cc5)c5cccc6c5ccc5ccccc56)c4)ccc3c3ccccc23)cc1 ; c1ccc(-c2c(-c3ccccc3)c3cc(-c4cccc(N(c5ccc(-c6ccc(-c7ccc8ccccc8c7)cc6)cc5)c5ccc(-c6cccc7ccccc67)cc5)c4)ccc3c3ccccc23)cc1 ; c1ccc(-c2c(-c3ccccc3)c3cc(-c4cccc(N(c5ccc(-c6ccc(-c7ccc8ccccc8c7)cc6)cc5)c5cccc6ccccc56)c4)ccc3c3ccccc23)cc1]

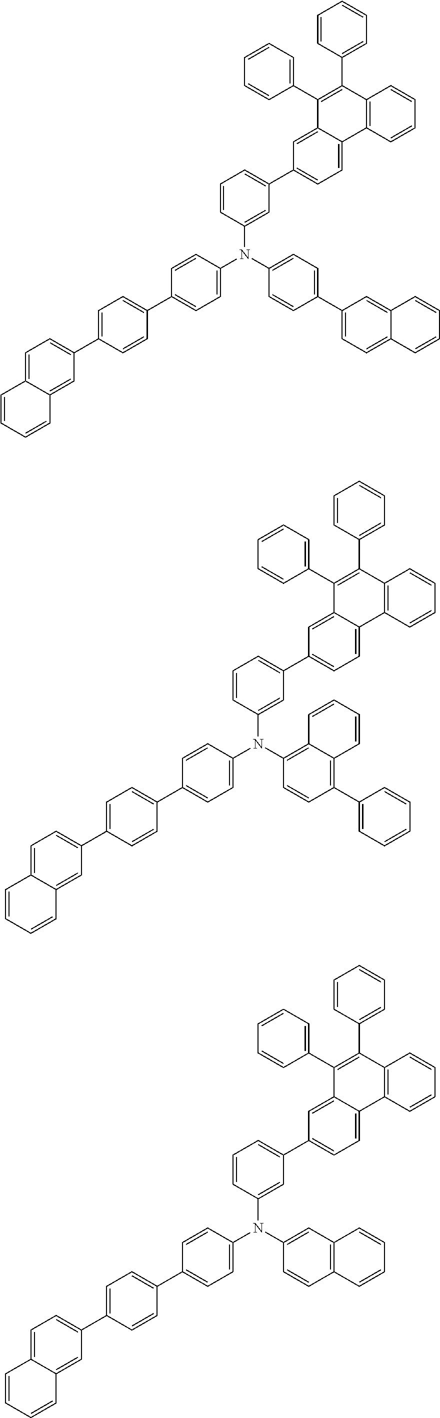 [SMILES: c1ccc(-c2c(-c3ccccc3)c3cc(-c4cccc(N(c5ccc(-c6ccc(-c7ccc8ccccc8c7)cc6)cc5)c5ccc(-c6ccc7ccccc7c6)cc5)c4)ccc3c3ccccc23)cc1 ; c1ccc(-c2ccc(N(c3ccc(-c4ccc(-c5ccc6ccccc6c5)cc4)cc3)c3cccc(-c4ccc5c(c4)c(-c4ccccc4)c(-c4ccccc4)c4ccccc45)c3)c3ccccc23)cc1 ; c1ccc(-c2c(-c3ccccc3)c3cc(-c4cccc(N(c5ccc(-c6ccc(-c7ccc8ccccc8c7)cc6)cc5)c5ccc6ccccc6c5)c4)ccc3c3ccccc23)cc1]

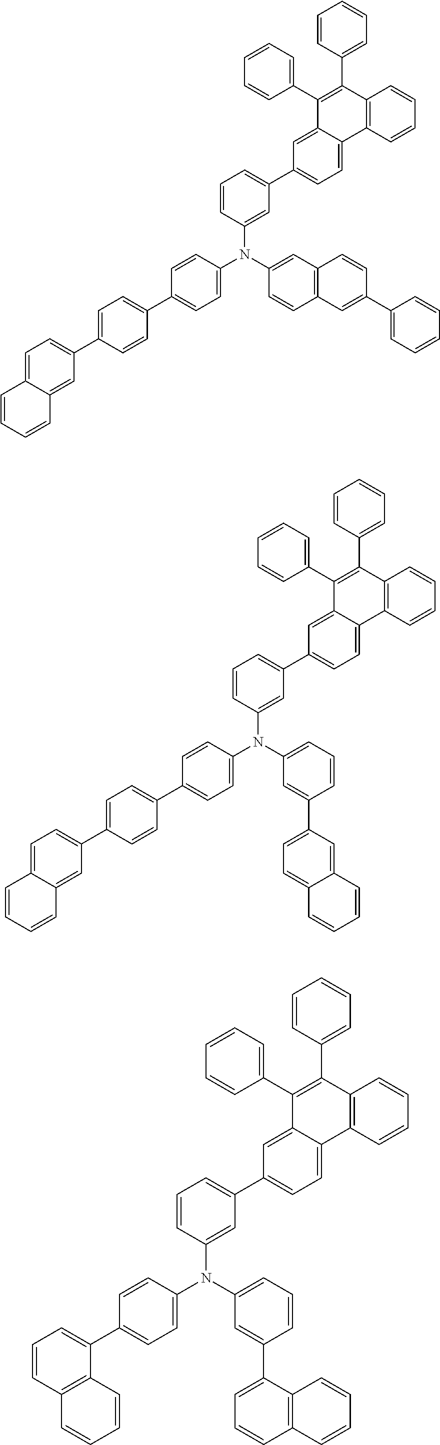 [SMILES: c1ccc(-c2ccc3cc(N(c4ccc(-c5ccc(-c6ccc7ccccc7c6)cc5)cc4)c4cccc(-c5ccc6c(c5)c(-c5ccccc5)c(-c5ccccc5)c5ccccc56)c4)ccc3c2)cc1 ; c1ccc(-c2c(-c3ccccc3)c3cc(-c4cccc(N(c5ccc(-c6ccc(-c7ccc8ccccc8c7)cc6)cc5)c5cccc(-c6ccc7ccccc7c6)c5)c4)ccc3c3ccccc23)cc1 ; c1ccc(-c2c(-c3ccccc3)c3cc(-c4cccc(N(c5ccc(-c6cccc7ccccc67)cc5)c5cccc(-c6cccc7ccccc67)c5)c4)ccc3c3ccccc23)cc1]

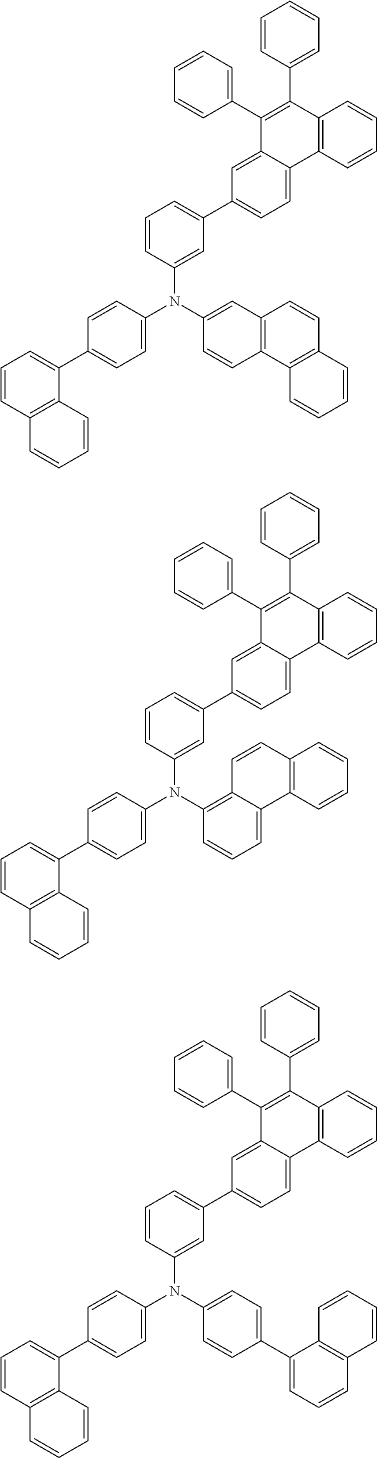 [SMILES: c1ccc(-c2c(-c3ccccc3)c3cc(-c4cccc(N(c5ccc(-c6cccc7ccccc67)cc5)c5ccc6c(ccc7ccccc76)c5)c4)ccc3c3ccccc23)cc1 ; c1ccc(-c2c(-c3ccccc3)c3cc(-c4cccc(N(c5ccc(-c6cccc7ccccc67)cc5)c5cccc6c5ccc5ccccc56)c4)ccc3c3ccccc23)cc1 ; c1ccc(-c2c(-c3ccccc3)c3cc(-c4cccc(N(c5ccc(-c6cccc7ccccc67)cc5)c5ccc(-c6cccc7ccccc67)cc5)c4)ccc3c3ccccc23)cc1]

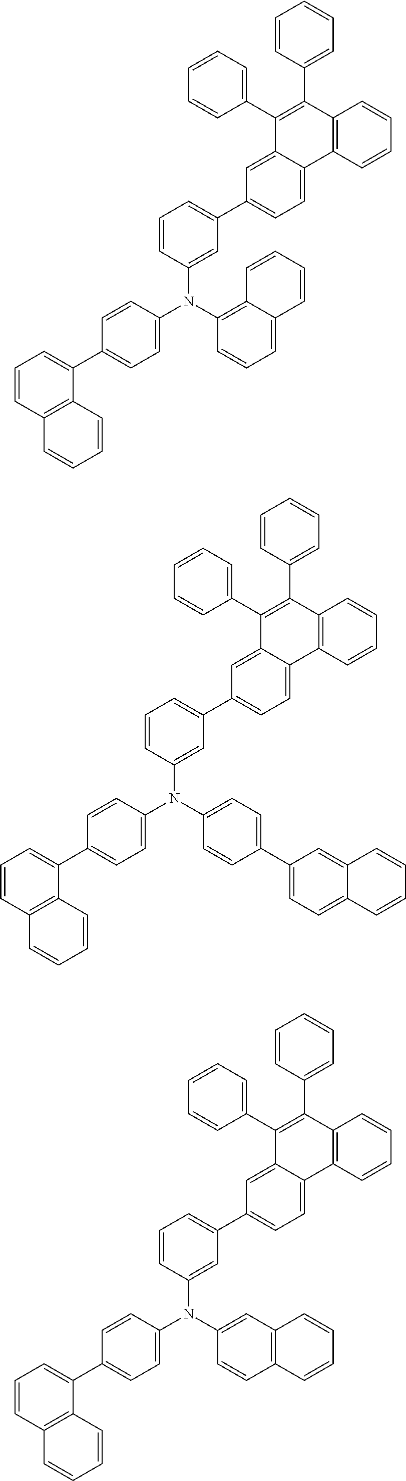 [SMILES: c1ccc(-c2c(-c3ccccc3)c3cc(-c4cccc(N(c5ccc(-c6cccc7ccccc67)cc5)c5cccc6ccccc56)c4)ccc3c3ccccc23)cc1 ; c1ccc(-c2c(-c3ccccc3)c3cc(-c4cccc(N(c5ccc(-c6ccc7ccccc7c6)cc5)c5ccc(-c6cccc7ccccc67)cc5)c4)ccc3c3ccccc23)cc1 ; c1ccc(-c2c(-c3ccccc3)c3cc(-c4cccc(N(c5ccc(-c6cccc7ccccc67)cc5)c5ccc6ccccc6c5)c4)ccc3c3ccccc23)cc1]

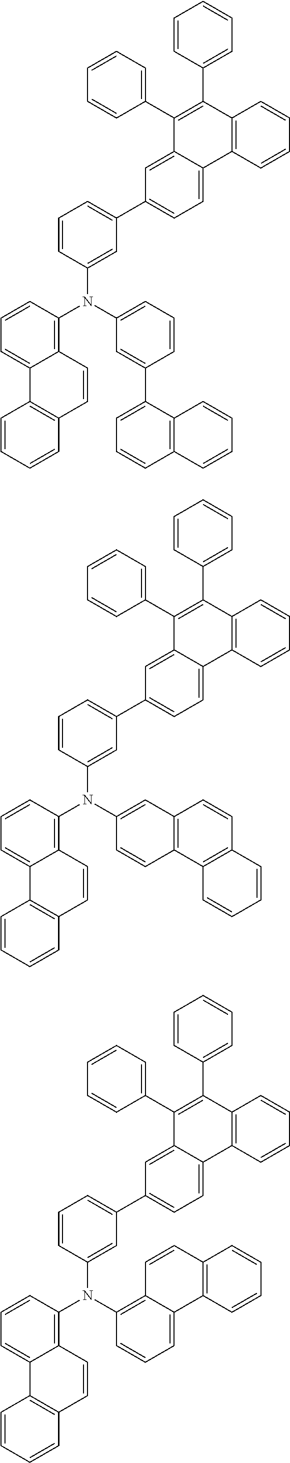

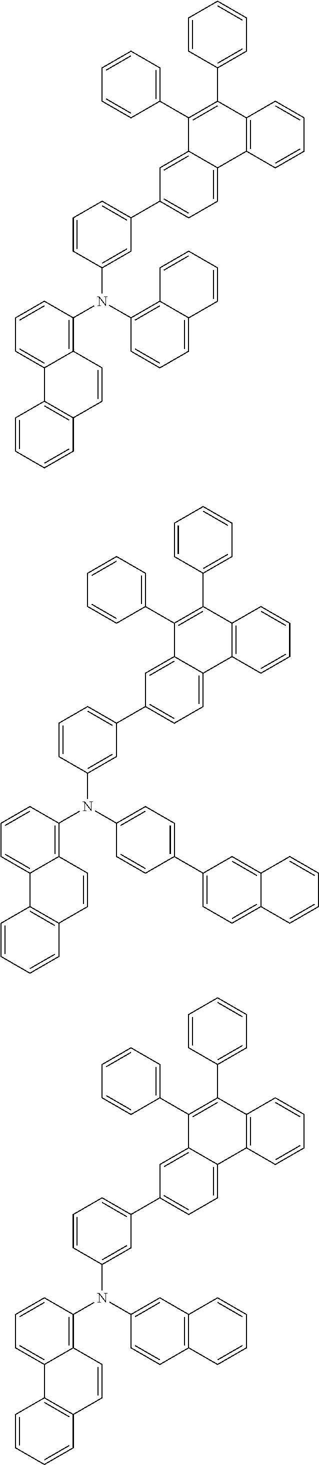 [SMILES: c1ccc(-c2c(-c3ccccc3)c3cc(-c4cccc(N(c5cccc6ccccc56)c5cccc6c5ccc5ccccc56)c4)ccc3c3ccccc23)cc1 ; c1ccc(-c2c(-c3ccccc3)c3cc(-c4cccc(N(c5ccc(-c6ccc7ccccc7c6)cc5)c5cccc6c5ccc5ccccc56)c4)ccc3c3ccccc23)cc1 ; c1ccc(-c2c(-c3ccccc3)c3cc(-c4cccc(N(c5ccc6ccccc6c5)c5cccc6c5ccc5ccccc56)c4)ccc3c3ccccc23)cc1]

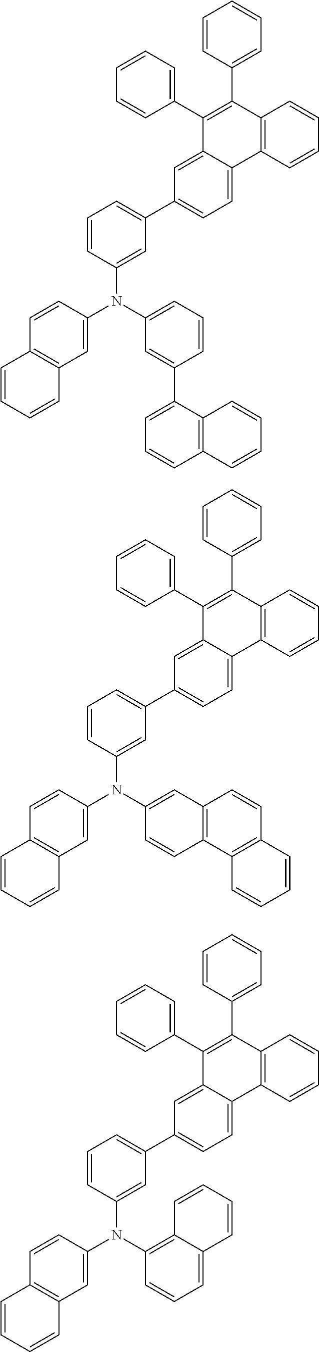

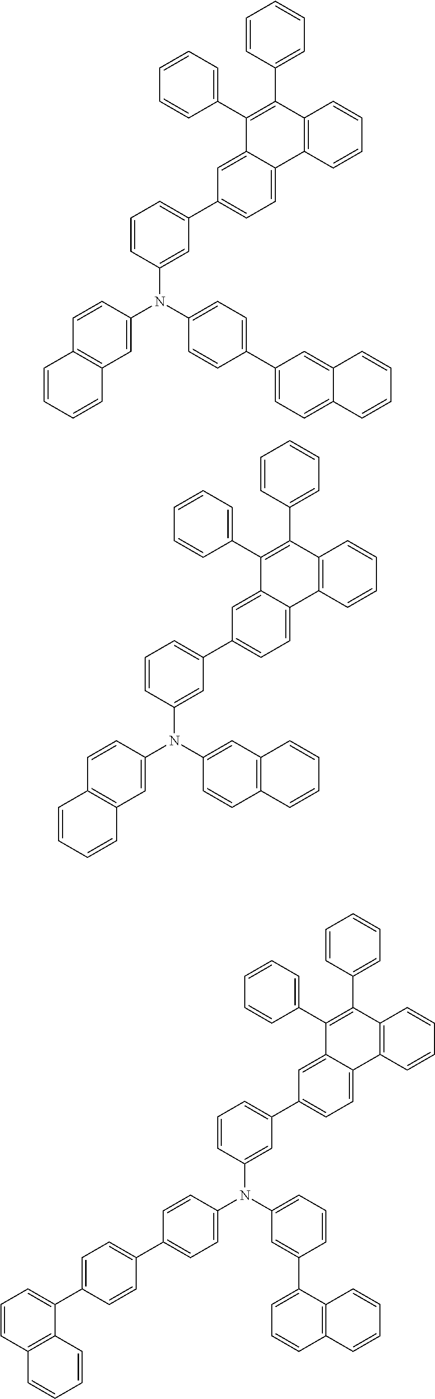

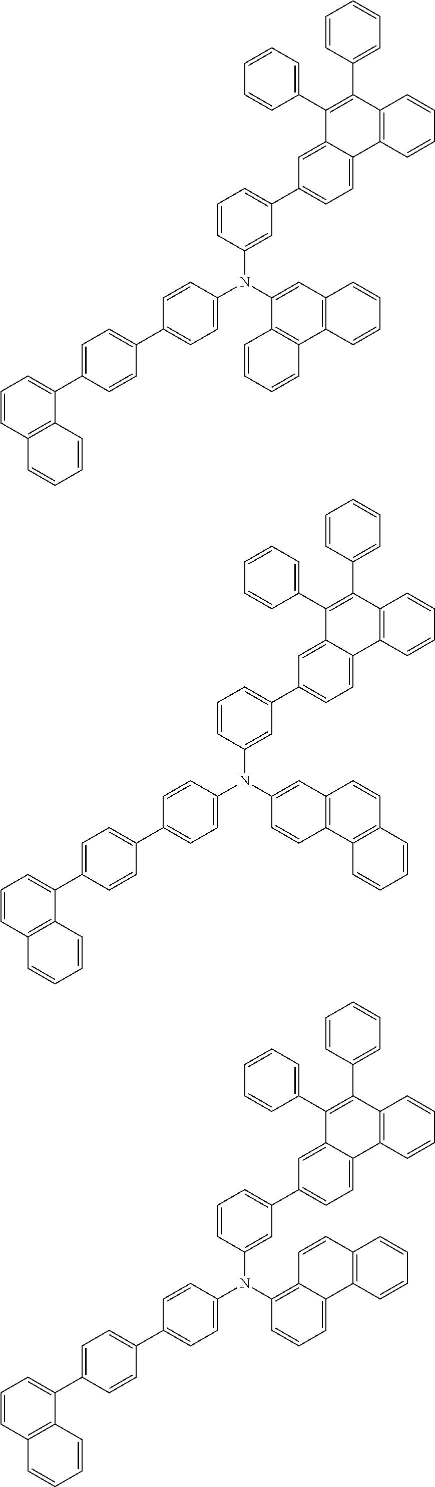 [SMILES: c1ccc(-c2c(-c3ccccc3)c3cc(-c4cccc(N(c5ccc(-c6ccc(-c7cccc8ccccc78)cc6)cc5)c5cc6ccccc6c6ccccc56)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5ccc6c(ccc7ccccc76)c5)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5cccc6c5ccc5ccccc56)c4)ccc3c3ccccc23)cc1]

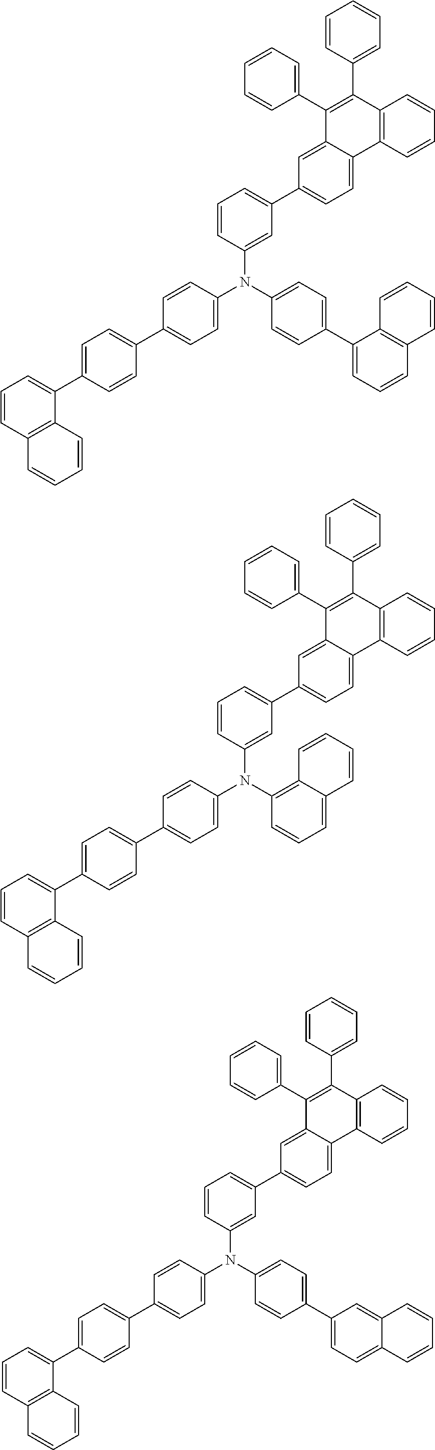 [SMILES: c1ccc(-c2c(-c3ccccc3)c3cc(-c4cccc(N(c5ccc(-c6ccc(-c7cccc8ccccc78)cc6)cc5)c5ccc(-c6cccc7ccccc67)cc5)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5cccc6ccccc56)c4)ccc3c3ccccc23)cc1 ; c1ccc(-c2c(-c3ccccc3)c3cc(-c4cccc(N(c5ccc(-c6ccc(-c7cccc8ccccc78)cc6)cc5)c5ccc(-c6ccc7ccccc7c6)cc5)c4)ccc3c3ccccc23)cc1]

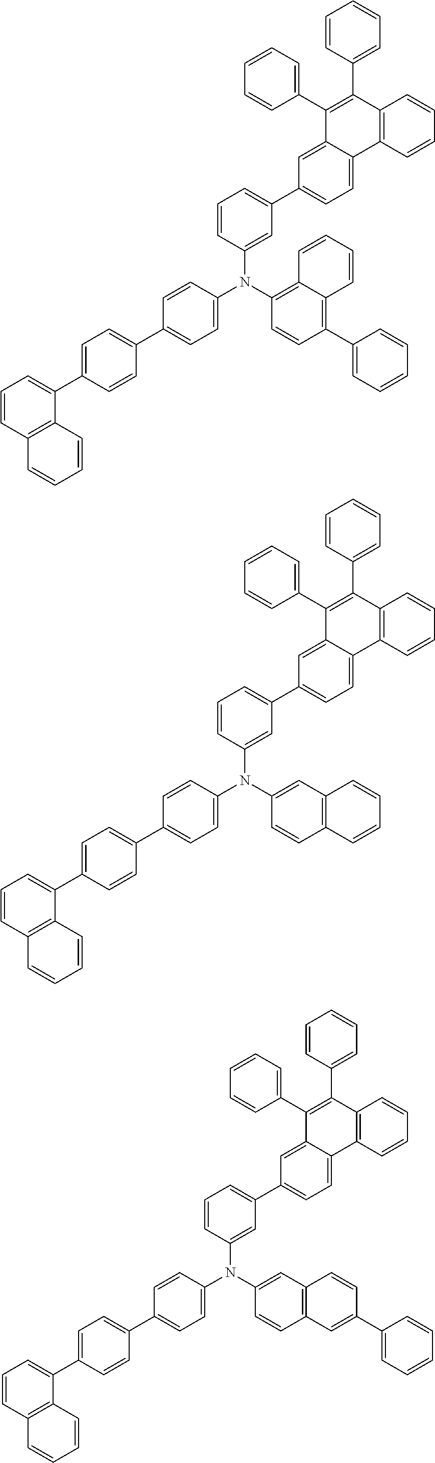 [SMILES: c1ccc(-c2ccc(N(c3ccc(-c4ccc(-c5cccc6ccccc56)cc4)cc3)c3cccc(-c4ccc5c(c4)c(-c4ccccc4)c(-c4ccccc4)c4ccccc45)c3)c3ccccc23)cc1 ; c1ccc(-c2c(-c3ccccc3)c3cc(-c4cccc(N(c5ccc(-c6ccc(-c7cccc8ccccc78)cc6)cc5)c5ccc6ccccc6c5)c4)ccc3c3ccccc23)cc1 ; c1ccc(-c2ccc3cc(N(c4ccc(-c5ccc(-c6cccc7ccccc67)cc5)cc4)c4cccc(-c5ccc6c(c5)c(-c5ccccc5)c(-c5ccccc5)c5ccccc56)c4)ccc3c2)cc1]

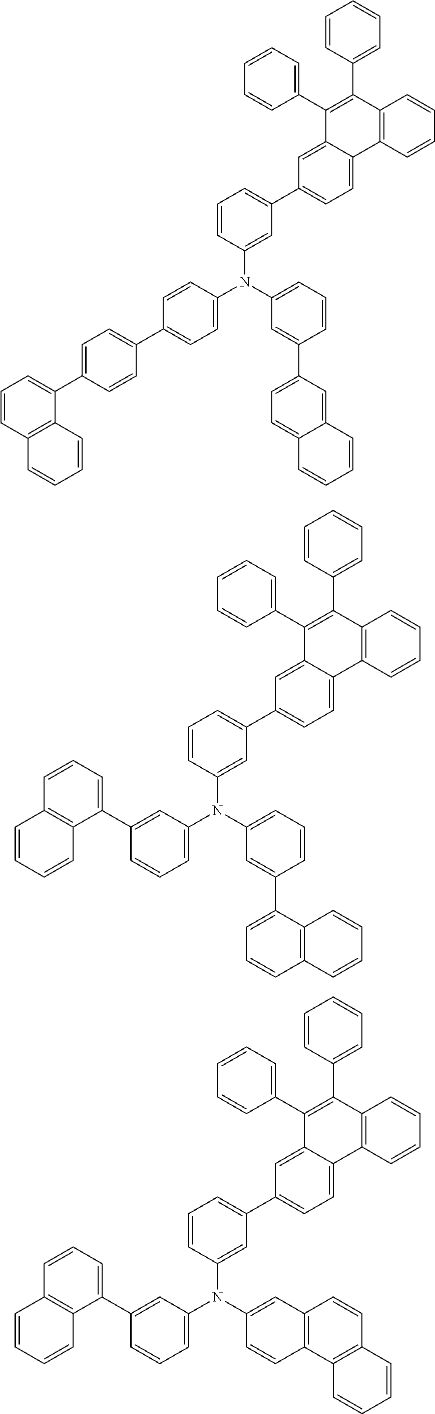

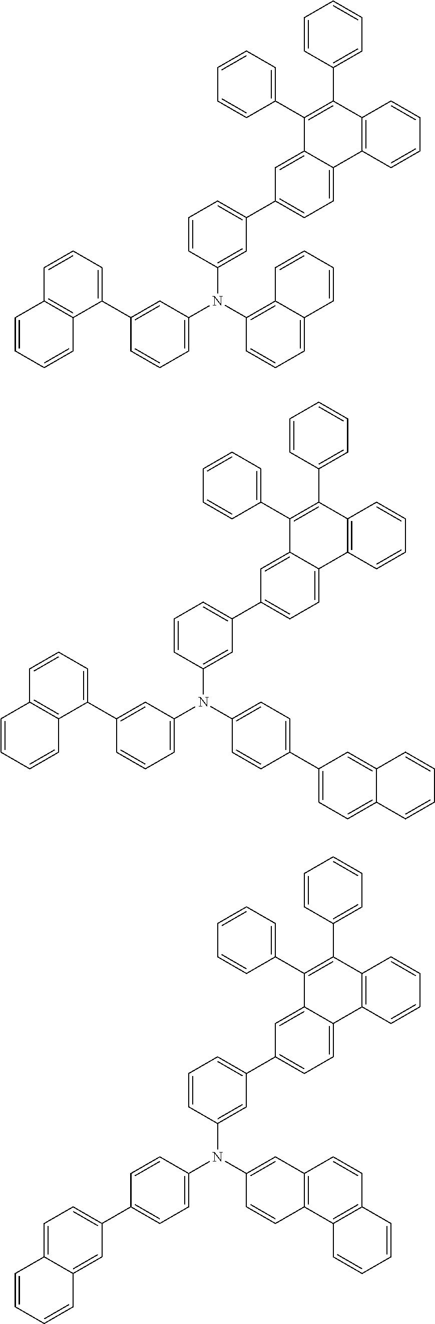

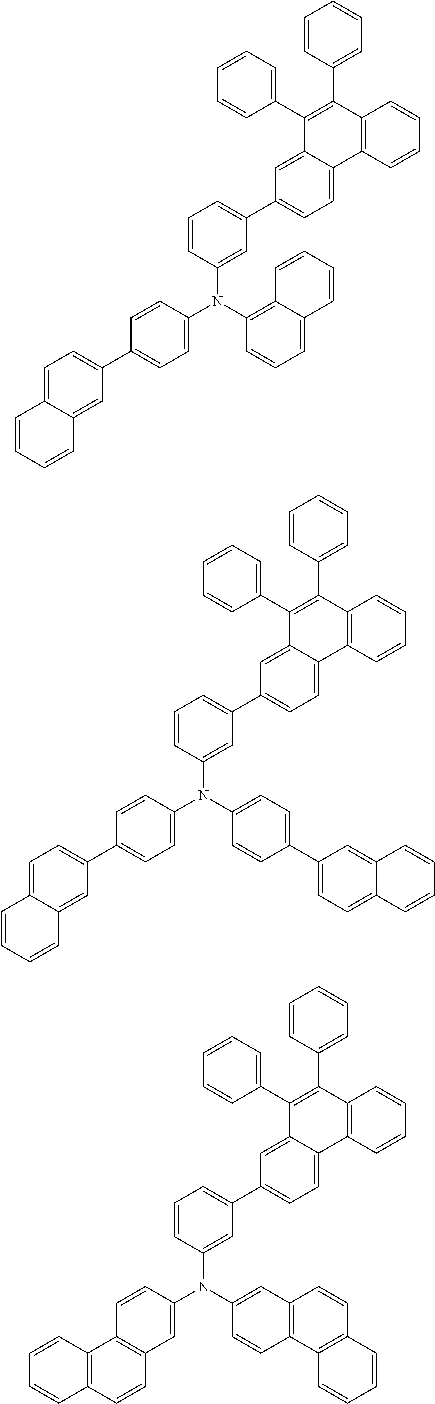 [SMILES: c1ccc(-c2c(-c3ccccc3)c3cc(-c4cccc(N(c5ccc(-c6ccc7ccccc7c6)cc5)c5cccc6ccccc56)c4)ccc3c3ccccc23)cc1 ; c1ccc(-c2c(-c3ccccc3)c3cc(-c4cccc(N(c5ccc(-c6ccc7ccccc7c6)cc5)c5ccc(-c6ccc7ccccc7c6)cc5)c4)ccc3c3ccccc23)cc1 ; c1ccc(-c2c(-c3ccccc3)c3cc(-c4cccc(N(c5ccc6c(ccc7ccccc76)c5)c5ccc6c(ccc7ccccc76)c5)c4)ccc3c3ccccc23)cc1]

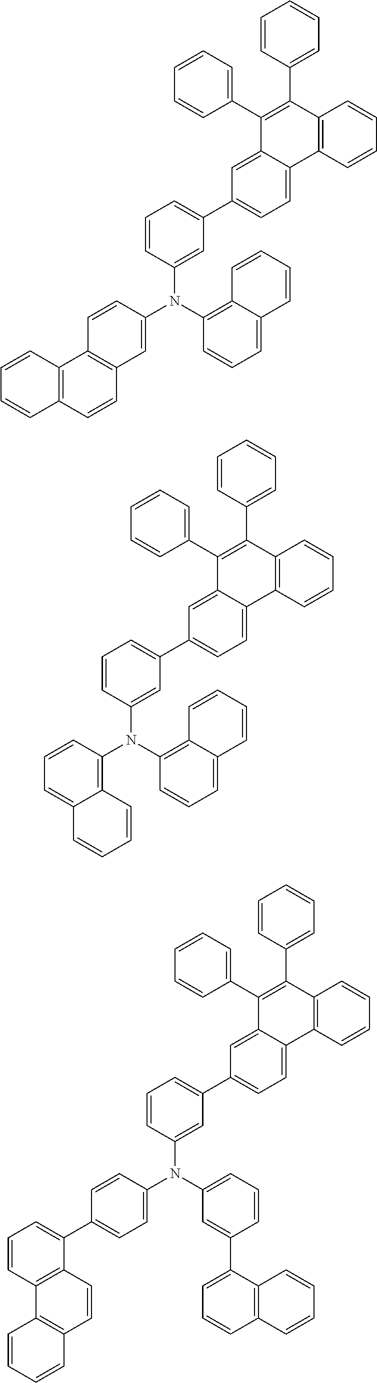 [SMILES: c1ccc(-c2c(-c3ccccc3)c3cc(-c4cccc(N(c5ccc6c(ccc7ccccc76)c5)c5cccc6ccccc56)c4)ccc3c3ccccc23)cc1 ; c1ccc(-c2c(-c3ccccc3)c3cc(-c4cccc(N(c5cccc6ccccc56)c5cccc6ccccc56)c4)ccc3c3ccccc23)cc1 ; c1ccc(-c2c(-c3ccccc3)c3cc(-c4cccc(N(c5ccc(-c6cccc7c6ccc6ccccc67)cc5)c5cccc(-c6cccc7ccccc67)c5)c4)ccc3c3ccccc23)cc1]

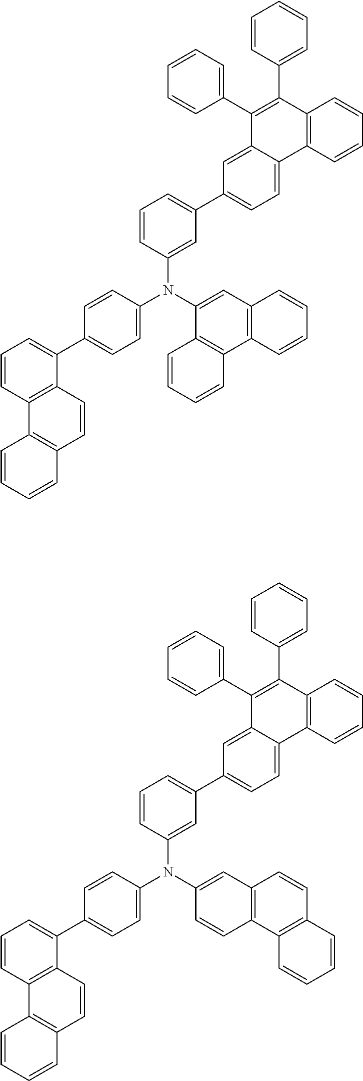 [SMILES: c1ccc(-c2c(-c3ccccc3)c3cc(-c4cccc(N(c5ccc(-c6cccc7c6ccc6ccccc67)cc5)c5cc6ccccc6c6ccccc56)c4)ccc3c3ccccc23)cc1 ; c1ccc(-c2c(-c3ccccc3)c3cc(-c4cccc(N(c5ccc(-c6cccc7c6ccc6ccccc67)cc5)c5ccc6c(ccc7ccccc76)c5)c4)ccc3c3ccccc23)cc1]

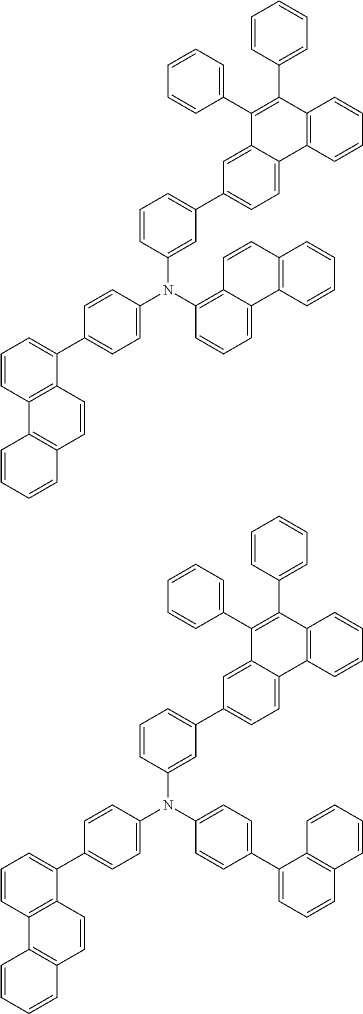 [SMILES: c1ccc(-c2c(-c3ccccc3)c3cc(-c4cccc(N(c5ccc(-c6cccc7c6ccc6ccccc67)cc5)c5cccc6c5ccc5ccccc56)c4)ccc3c3ccccc23)cc1 ; c1ccc(-c2c(-c3ccccc3)c3cc(-c4cccc(N(c5ccc(-c6cccc7ccccc67)cc5)c5ccc(-c6cccc7c6ccc6ccccc67)cc5)c4)ccc3c3ccccc23)cc1]

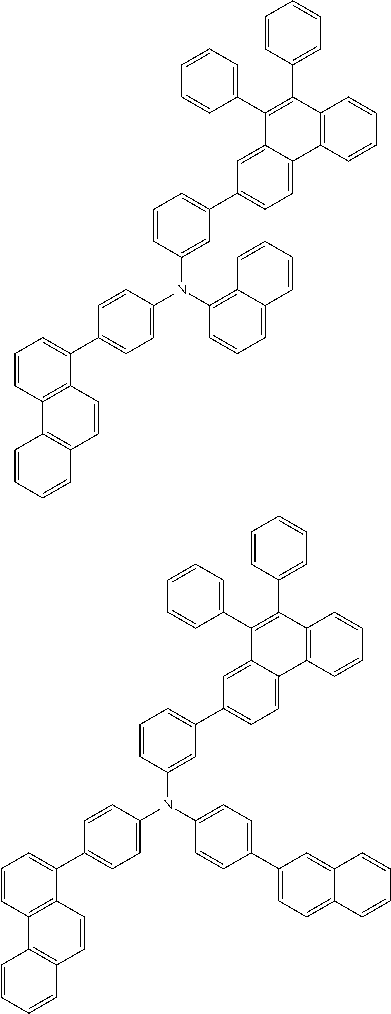 [SMILES: c1ccc(-c2c(-c3ccccc3)c3cc(-c4cccc(N(c5ccc(-c6cccc7c6ccc6ccccc67)cc5)c5cccc6ccccc56)c4)ccc3c3ccccc23)cc1 ; c1ccc(-c2c(-c3ccccc3)c3cc(-c4cccc(N(c5ccc(-c6ccc7ccccc7c6)cc5)c5ccc(-c6cccc7c6ccc6ccccc67)cc5)c4)ccc3c3ccccc23)cc1]

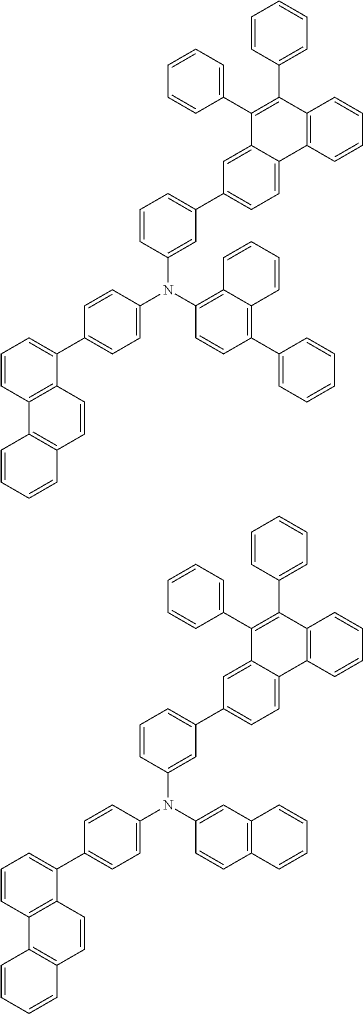 [SMILES: c1ccc(-c2ccc(N(c3ccc(-c4cccc5c4ccc4ccccc45)cc3)c3cccc(-c4ccc5c(c4)c(-c4ccccc4)c(-c4ccccc4)c4ccccc45)c3)c3ccccc23)cc1 ; c1ccc(-c2c(-c3ccccc3)c3cc(-c4cccc(N(c5ccc(-c6cccc7c6ccc6ccccc67)cc5)c5ccc6ccccc6c5)c4)ccc3c3ccccc23)cc1]

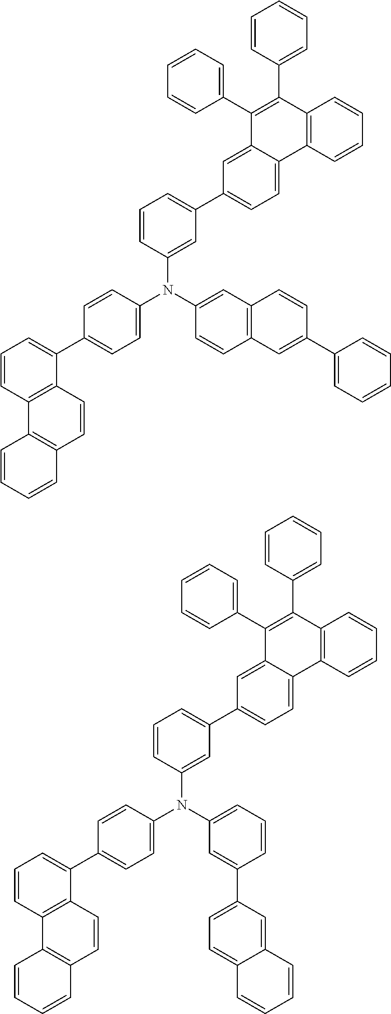 [SMILES: c1ccc(-c2ccc3cc(N(c4ccc(-c5cccc6c5ccc5ccccc56)cc4)c4cccc(-c5ccc6c(c5)c(-c5ccccc5)c(-c5ccccc5)c5ccccc56)c4)ccc3c2)cc1 ; c1ccc(-c2c(-c3ccccc3)c3cc(-c4cccc(N(c5ccc(-c6cccc7c6ccc6ccccc67)cc5)c5cccc(-c6ccc7ccccc7c6)c5)c4)ccc3c3ccccc23)cc1]

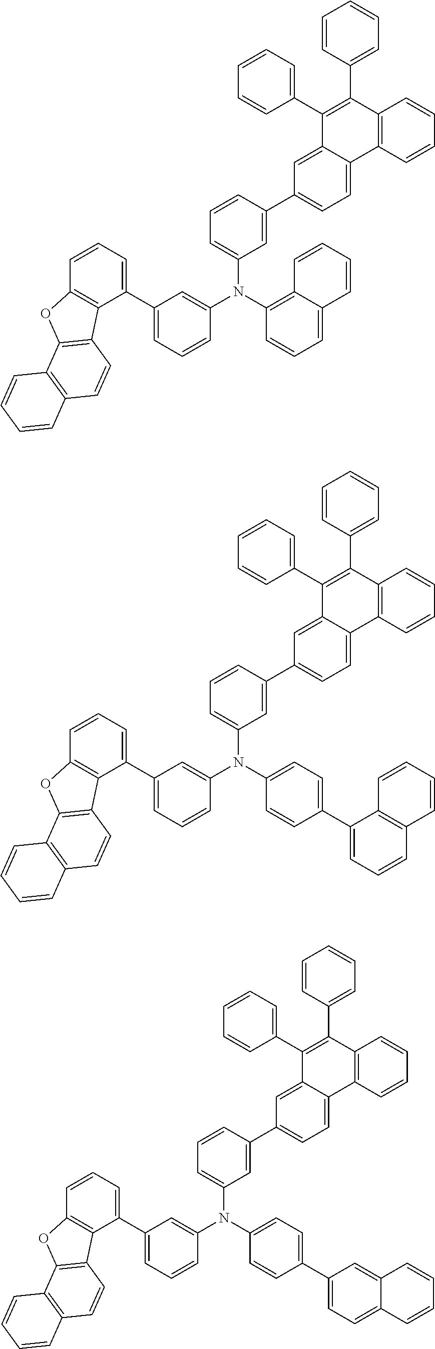 [SMILES: c1ccc(-c2c(-c3ccccc3)c3cc(-c4cccc(N(c5cccc(-c6cccc7oc8c9ccccc9ccc8c67)c5)c5cccc6ccccc56)c4)ccc3c3ccccc23)cc1 ; c1ccc(-c2c(-c3ccccc3)c3cc(-c4cccc(N(c5ccc(-c6cccc7ccccc67)cc5)c5cccc(-c6cccc7oc8c9ccccc9ccc8c67)c5)c4)ccc3c3ccccc23)cc1 ; c1ccc(-c2c(-c3ccccc3)c3cc(-c4cccc(N(c5ccc(-c6ccc7ccccc7c6)cc5)c5cccc(-c6cccc7oc8c9ccccc9ccc8c67)c5)c4)ccc3c3ccccc23)cc1]

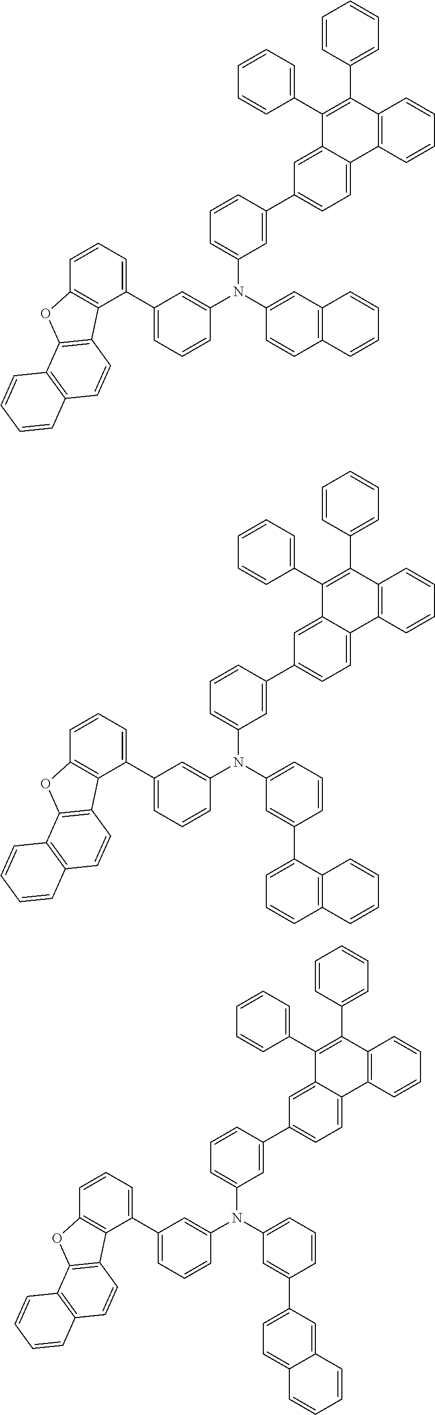

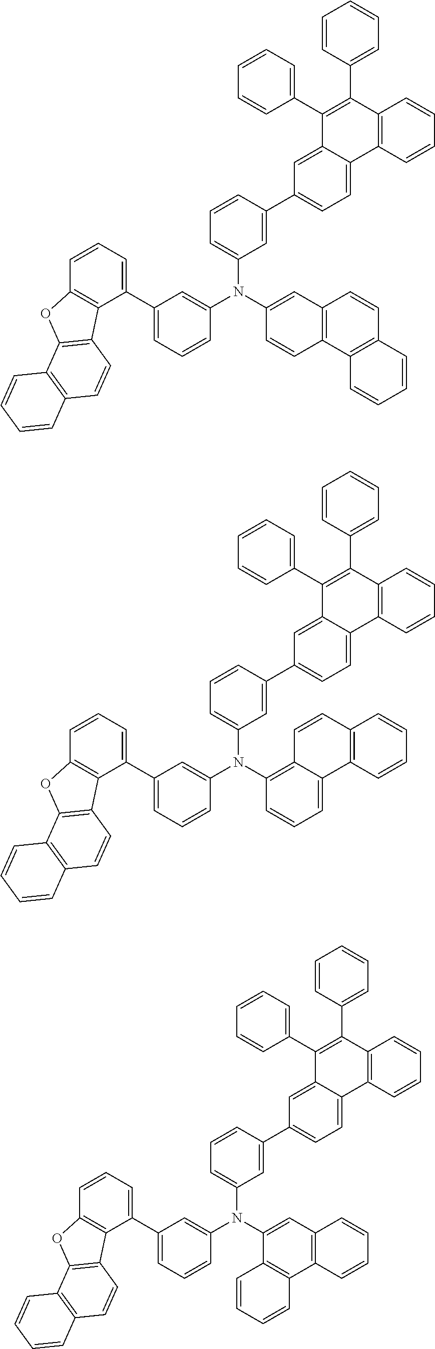 [SMILES: c1ccc(-c2c(-c3ccccc3)c3cc(-c4cccc(N(c5cccc(-c6cccc7oc8c9ccccc9ccc8c67)c5)c5ccc6c(ccc7ccccc76)c5)c4)ccc3c3ccccc23)cc1 ; c1ccc(-c2c(-c3ccccc3)c3cc(-c4cccc(N(c5cccc(-c6cccc7oc8c9ccccc9ccc8c67)c5)c5cccc6c5ccc5ccccc56)c4)ccc3c3ccccc23)cc1 ; c1ccc(-c2c(-c3ccccc3)c3cc(-c4cccc(N(c5cccc(-c6cccc7oc8c9ccccc9ccc8c67)c5)c5cc6ccccc6c6ccccc56)c4)ccc3c3ccccc23)cc1]

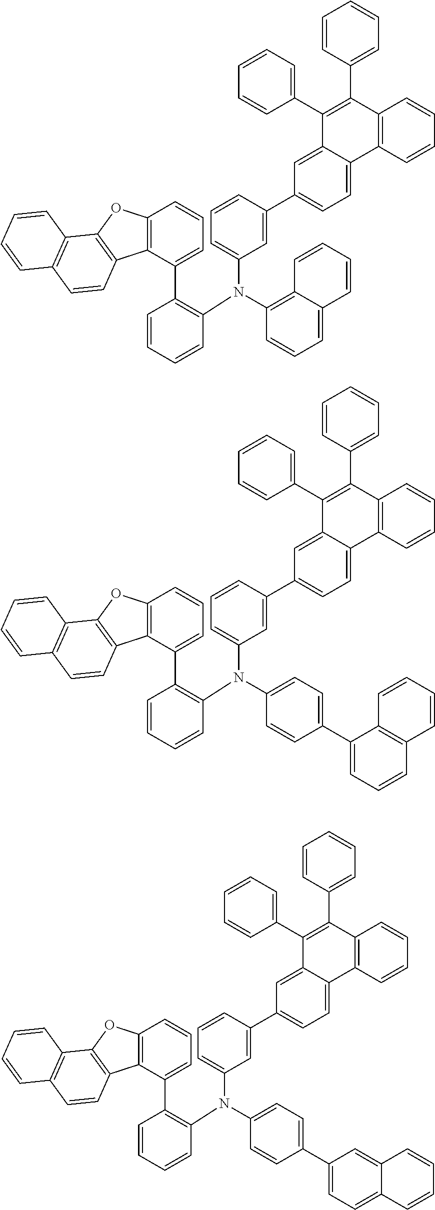 [SMILES: c1ccc(-c2c(-c3ccccc3)c3cc(-c4cccc(N(c5ccccc5-c5cccc6oc7c8ccccc8ccc7c56)c5cccc6ccccc56)c4)ccc3c3ccccc23)cc1 ; c1ccc(-c2c(-c3ccccc3)c3cc(-c4cccc(N(c5ccc(-c6cccc7ccccc67)cc5)c5ccccc5-c5cccc6oc7c8ccccc8ccc7c56)c4)ccc3c3ccccc23)cc1 ; c1ccc(-c2c(-c3ccccc3)c3cc(-c4cccc(N(c5ccc(-c6ccc7ccccc7c6)cc5)c5ccccc5-c5cccc6oc7c8ccccc8ccc7c56)c4)ccc3c3ccccc23)cc1]

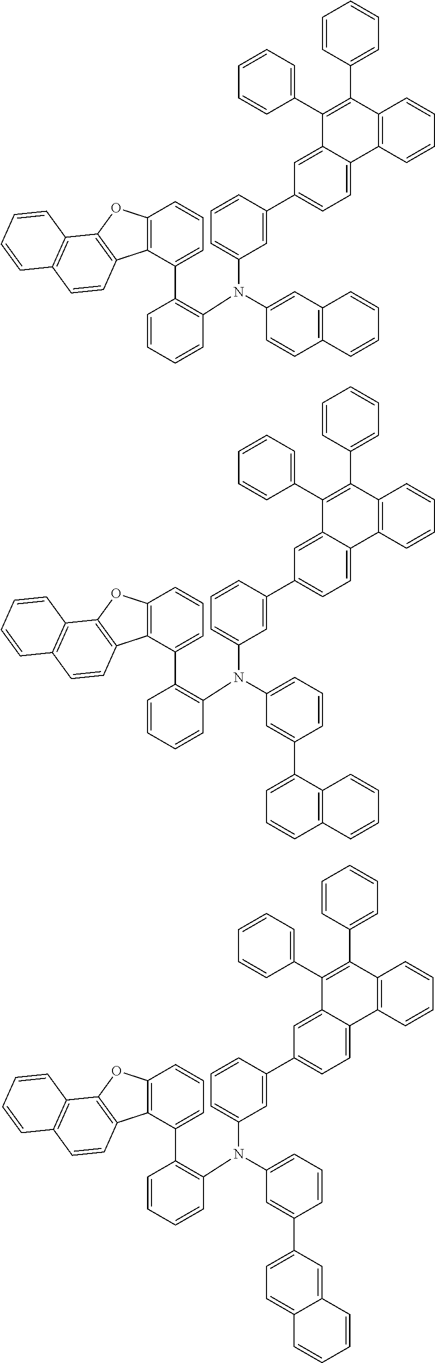

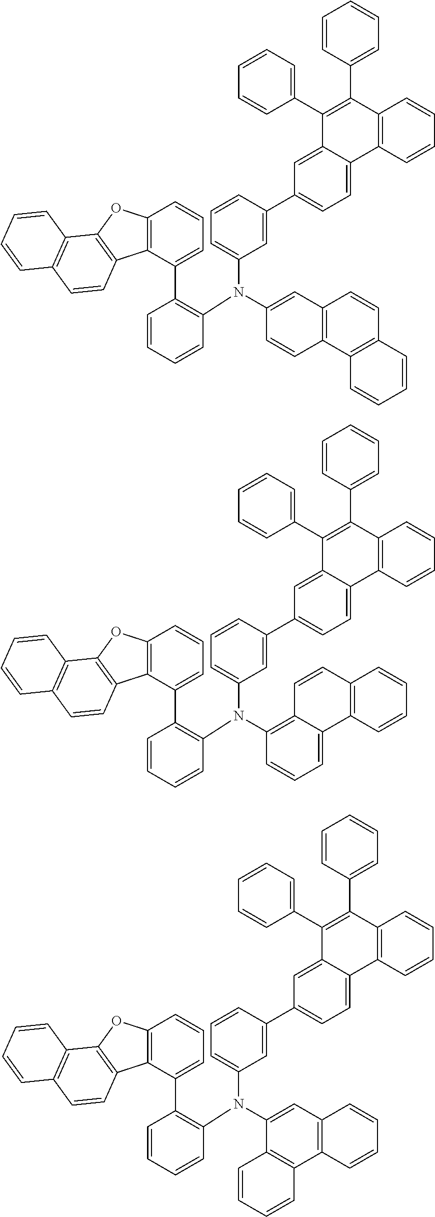

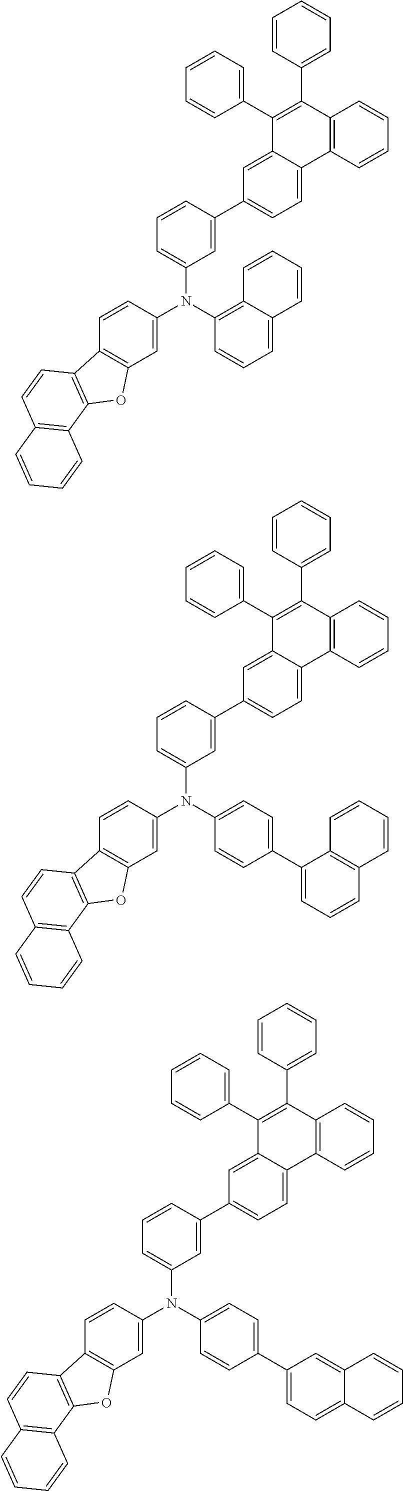 [SMILES: c1ccc(-c2c(-c3ccccc3)c3cc(-c4cccc(N(c5ccc6c(c5)oc5c7ccccc7ccc65)c5cccc6ccccc56)c4)ccc3c3ccccc23)cc1 ; c1ccc(-c2c(-c3ccccc3)c3cc(-c4cccc(N(c5ccc(-c6cccc7ccccc67)cc5)c5ccc6c(c5)oc5c7ccccc7ccc65)c4)ccc3c3ccccc23)cc1 ; c1ccc(-c2c(-c3ccccc3)c3cc(-c4cccc(N(c5ccc(-c6ccc7ccccc7c6)cc5)c5ccc6c(c5)oc5c7ccccc7ccc65)c4)ccc3c3ccccc23)cc1]

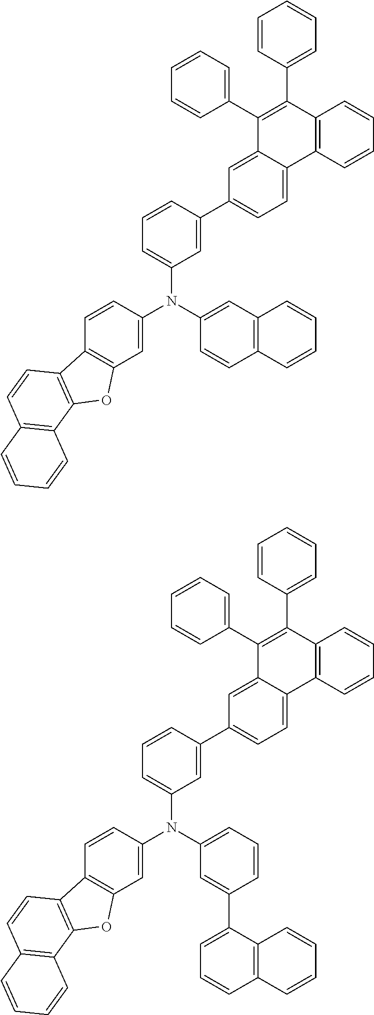 [SMILES: c1ccc(-c2c(-c3ccccc3)c3cc(-c4cccc(N(c5ccc6ccccc6c5)c5ccc6c(c5)oc5c7ccccc7ccc65)c4)ccc3c3ccccc23)cc1 ; c1ccc(-c2c(-c3ccccc3)c3cc(-c4cccc(N(c5cccc(-c6cccc7ccccc67)c5)c5ccc6c(c5)oc5c7ccccc7ccc65)c4)ccc3c3ccccc23)cc1]

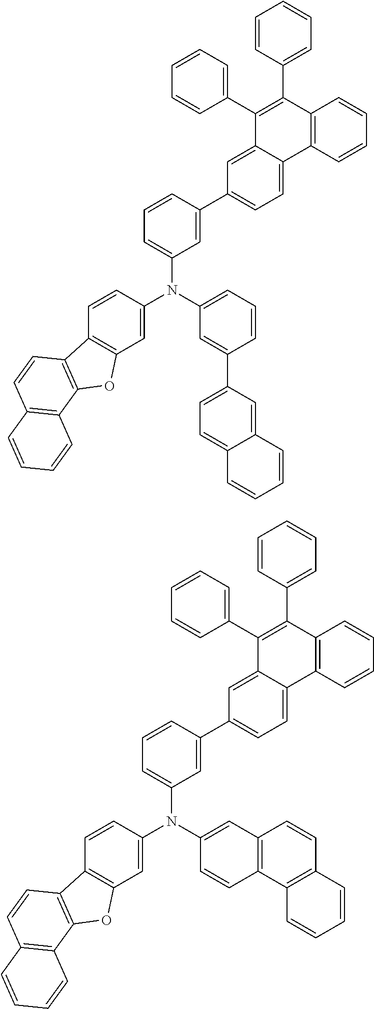

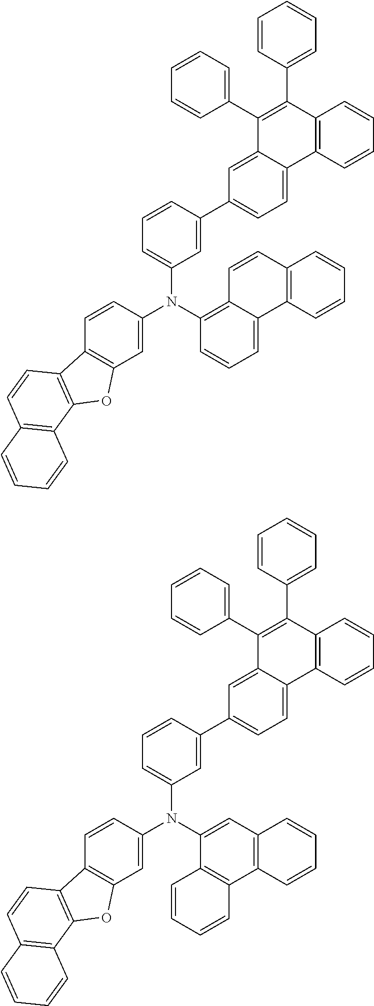 [SMILES: c1ccc(-c2c(-c3ccccc3)c3cc(-c4cccc(N(c5ccc6c(c5)oc5c7ccccc7ccc65)c5cccc6c5ccc5ccccc56)c4)ccc3c3ccccc23)cc1 ; c1ccc(-c2c(-c3ccccc3)c3cc(-c4cccc(N(c5ccc6c(c5)oc5c7ccccc7ccc65)c5cc6ccccc6c6ccccc56)c4)ccc3c3ccccc23)cc1]

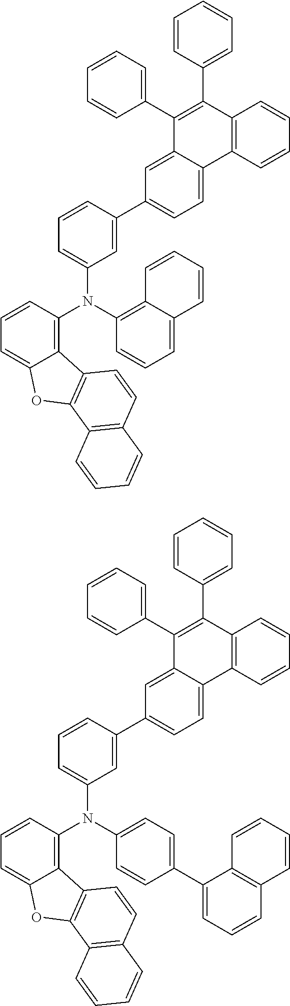 [SMILES: c1ccc(-c2c(-c3ccccc3)c3cc(-c4cccc(N(c5cccc6ccccc56)c5cccc6oc7c8ccccc8ccc7c56)c4)ccc3c3ccccc23)cc1 ; c1ccc(-c2c(-c3ccccc3)c3cc(-c4cccc(N(c5ccc(-c6cccc7ccccc67)cc5)c5cccc6oc7c8ccccc8ccc7c56)c4)ccc3c3ccccc23)cc1]

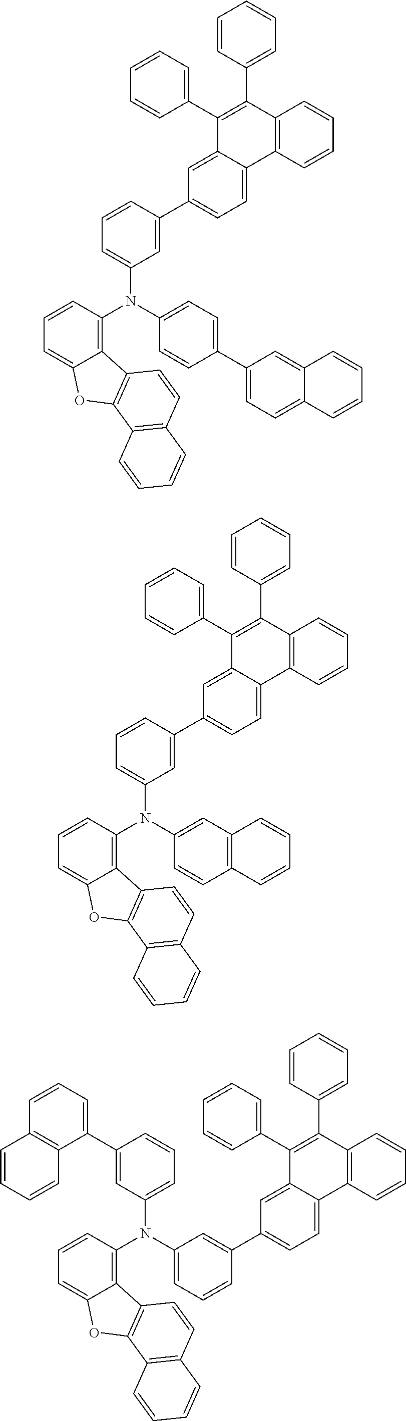 [SMILES: c1ccc(-c2c(-c3ccccc3)c3cc(-c4cccc(N(c5ccc(-c6ccc7ccccc7c6)cc5)c5cccc6oc7c8ccccc8ccc7c56)c4)ccc3c3ccccc23)cc1 ; c1ccc(-c2c(-c3ccccc3)c3cc(-c4cccc(N(c5ccc6ccccc6c5)c5cccc6oc7c8ccccc8ccc7c56)c4)ccc3c3ccccc23)cc1 ; c1ccc(-c2c(-c3ccccc3)c3cc(-c4cccc(N(c5cccc(-c6cccc7ccccc67)c5)c5cccc6oc7c8ccccc8ccc7c56)c4)ccc3c3ccccc23)cc1]

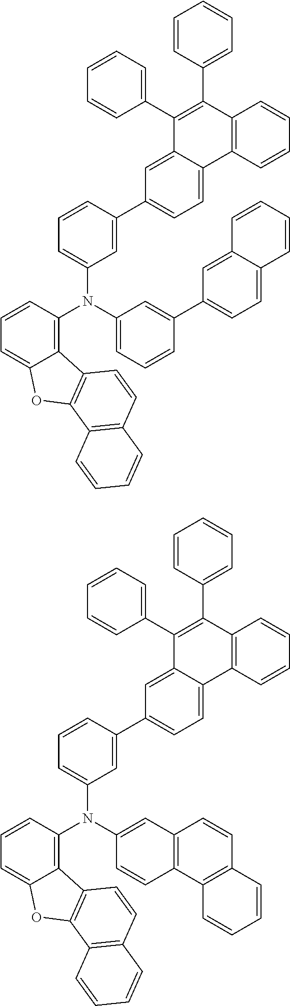 [SMILES: c1ccc(-c2c(-c3ccccc3)c3cc(-c4cccc(N(c5cccc(-c6ccc7ccccc7c6)c5)c5cccc6oc7c8ccccc8ccc7c56)c4)ccc3c3ccccc23)cc1 ; c1ccc(-c2c(-c3ccccc3)c3cc(-c4cccc(N(c5ccc6c(ccc7ccccc76)c5)c5cccc6oc7c8ccccc8ccc7c56)c4)ccc3c3ccccc23)cc1]

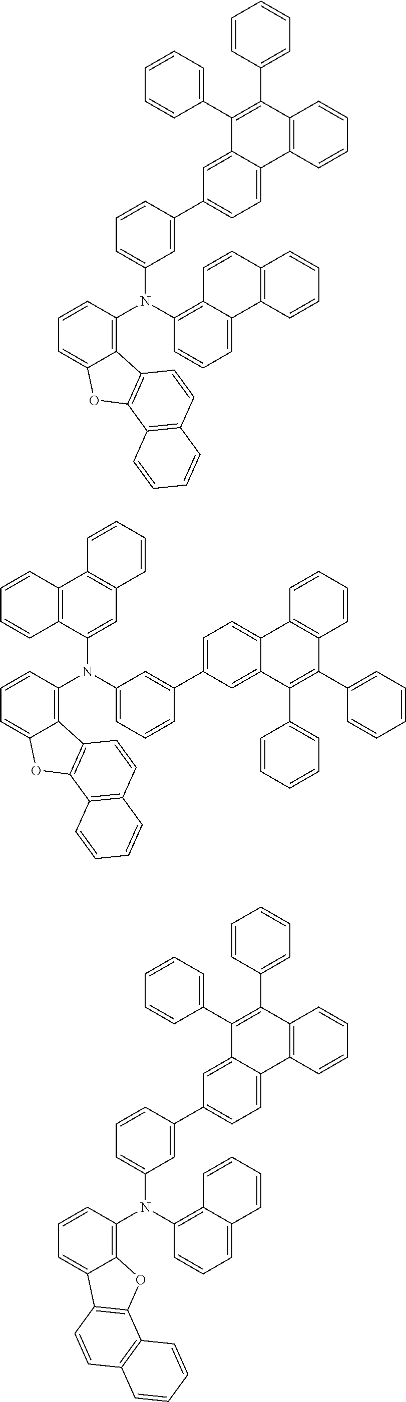 [SMILES: c1ccc(-c2c(-c3ccccc3)c3cc(-c4cccc(N(c5cccc6c5ccc5ccccc56)c5cccc6oc7c8ccccc8ccc7c56)c4)ccc3c3ccccc23)cc1 ; c1ccc(-c2c(-c3ccccc3)c3cc(-c4cccc(N(c5cc6ccccc6c6ccccc56)c5cccc6oc7c8ccccc8ccc7c56)c4)ccc3c3ccccc23)cc1 ; c1ccc(-c2c(-c3ccccc3)c3cc(-c4cccc(N(c5cccc6ccccc56)c5cccc6c5oc5c7ccccc7ccc65)c4)ccc3c3ccccc23)cc1]

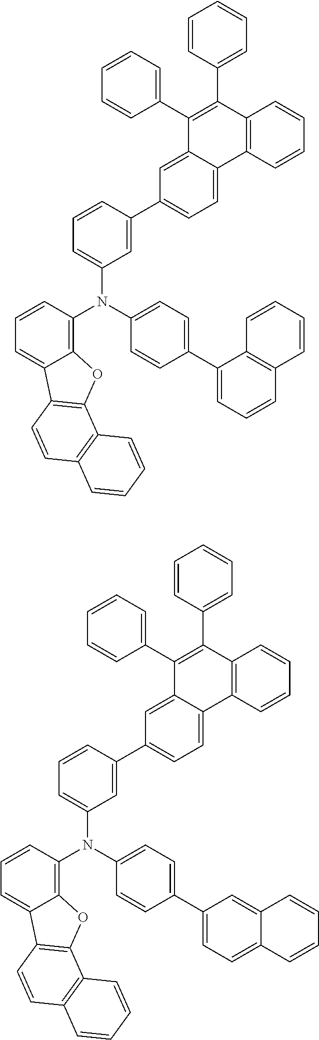 [SMILES: c1ccc(-c2c(-c3ccccc3)c3cc(-c4cccc(N(c5ccc(-c6cccc7ccccc67)cc5)c5cccc6c5oc5c7ccccc7ccc65)c4)ccc3c3ccccc23)cc1 ; c1ccc(-c2c(-c3ccccc3)c3cc(-c4cccc(N(c5ccc(-c6ccc7ccccc7c6)cc5)c5cccc6c5oc5c7ccccc7ccc65)c4)ccc3c3ccccc23)cc1]

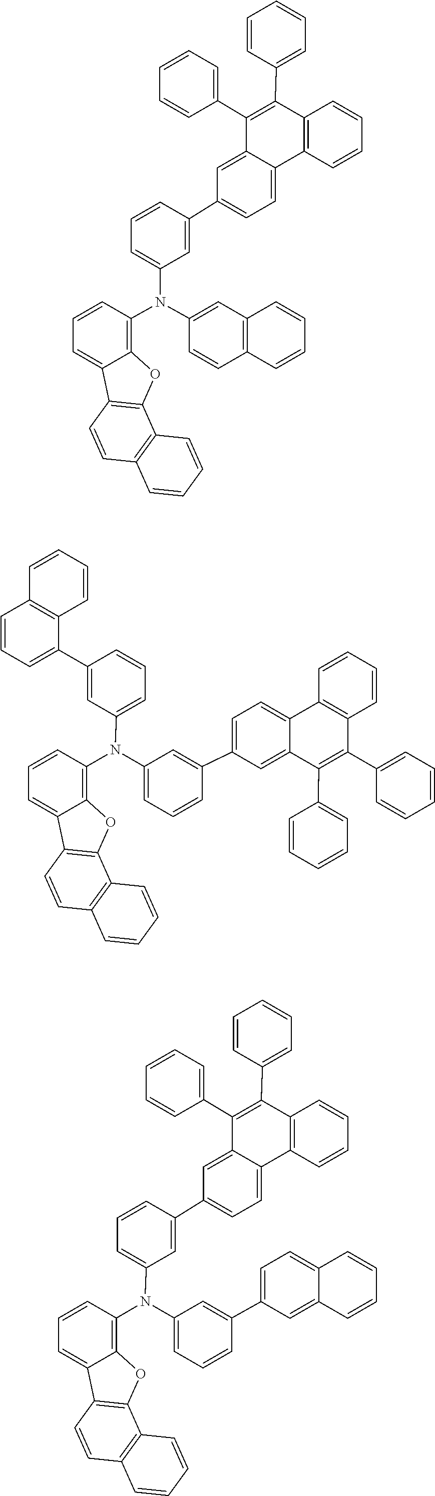 [SMILES: c1ccc(-c2c(-c3ccccc3)c3cc(-c4cccc(N(c5ccc6ccccc6c5)c5cccc6c5oc5c7ccccc7ccc65)c4)ccc3c3ccccc23)cc1 ; c1ccc(-c2c(-c3ccccc3)c3cc(-c4cccc(N(c5cccc(-c6cccc7ccccc67)c5)c5cccc6c5oc5c7ccccc7ccc65)c4)ccc3c3ccccc23)cc1 ; c1ccc(-c2c(-c3ccccc3)c3cc(-c4cccc(N(c5cccc(-c6ccc7ccccc7c6)c5)c5cccc6c5oc5c7ccccc7ccc65)c4)ccc3c3ccccc23)cc1]

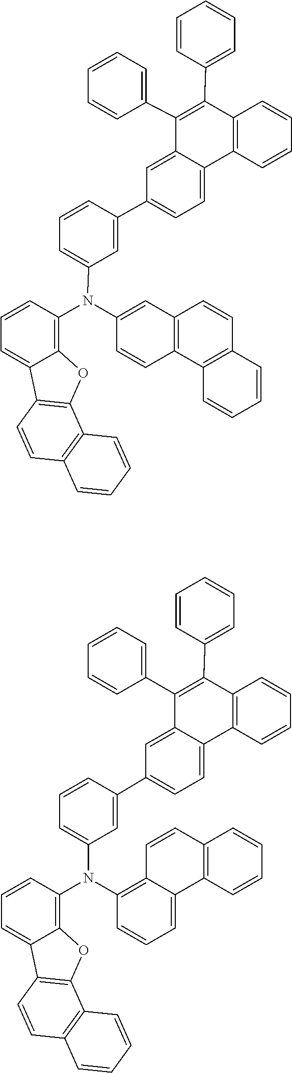 [SMILES: c1ccc(-c2c(-c3ccccc3)c3cc(-c4cccc(N(c5ccc6c(ccc7ccccc76)c5)c5cccc6c5oc5c7ccccc7ccc65)c4)ccc3c3ccccc23)cc1 ; c1ccc(-c2c(-c3ccccc3)c3cc(-c4cccc(N(c5cccc6c5ccc5ccccc56)c5cccc6c5oc5c7ccccc7ccc65)c4)ccc3c3ccccc23)cc1]

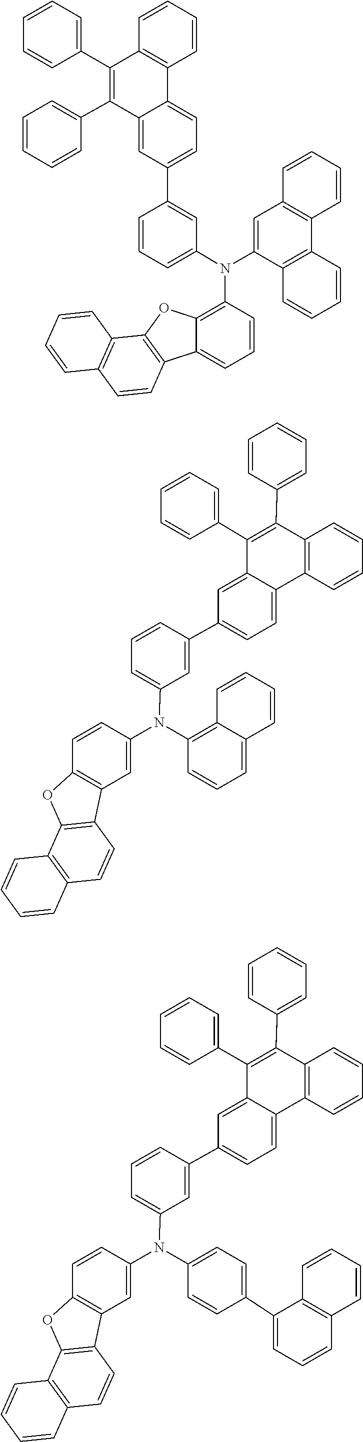 [SMILES: c1ccc(-c2c(-c3ccccc3)c3cc(-c4cccc(N(c5cc6ccccc6c6ccccc56)c5cccc6c5oc5c7ccccc7ccc65)c4)ccc3c3ccccc23)cc1 ; c1ccc(-c2c(-c3ccccc3)c3cc(-c4cccc(N(c5ccc6oc7c8ccccc8ccc7c6c5)c5cccc6ccccc56)c4)ccc3c3ccccc23)cc1 ; c1ccc(-c2c(-c3ccccc3)c3cc(-c4cccc(N(c5ccc(-c6cccc7ccccc67)cc5)c5ccc6oc7c8ccccc8ccc7c6c5)c4)ccc3c3ccccc23)cc1]

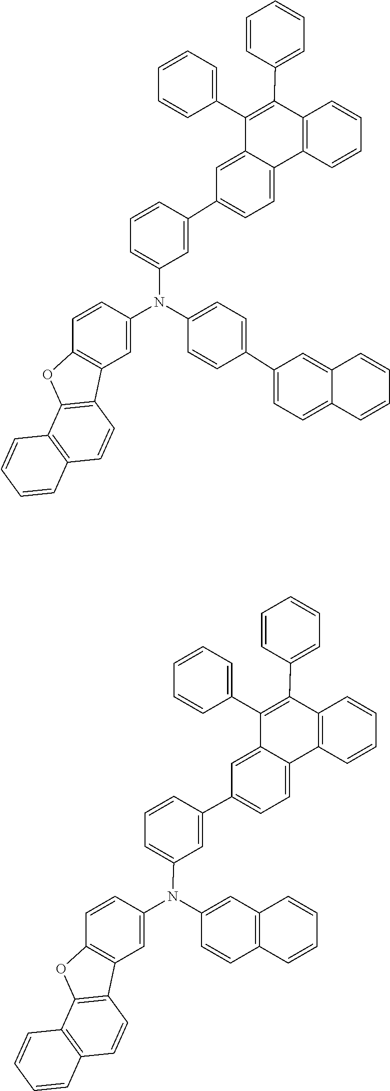 [SMILES: c1ccc(-c2c(-c3ccccc3)c3cc(-c4cccc(N(c5ccc(-c6ccc7ccccc7c6)cc5)c5ccc6oc7c8ccccc8ccc7c6c5)c4)ccc3c3ccccc23)cc1 ; c1ccc(-c2c(-c3ccccc3)c3cc(-c4cccc(N(c5ccc6ccccc6c5)c5ccc6oc7c8ccccc8ccc7c6c5)c4)ccc3c3ccccc23)cc1]

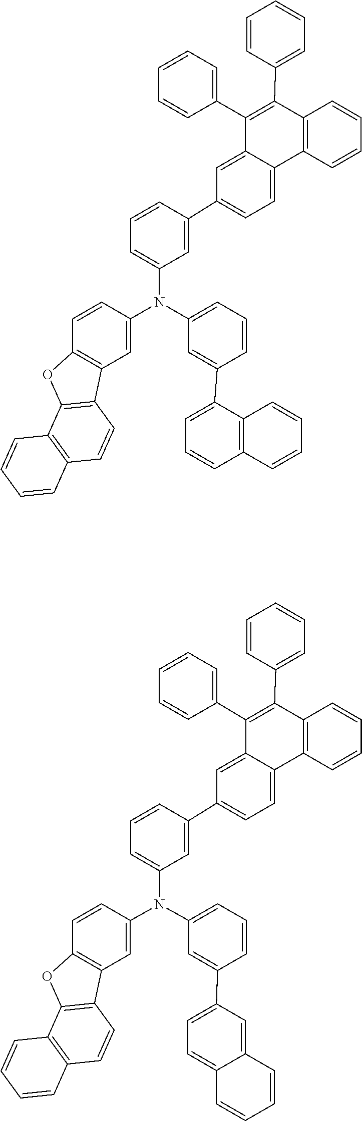 [SMILES: c1ccc(-c2c(-c3ccccc3)c3cc(-c4cccc(N(c5cccc(-c6cccc7ccccc67)c5)c5ccc6oc7c8ccccc8ccc7c6c5)c4)ccc3c3ccccc23)cc1 ; c1ccc(-c2c(-c3ccccc3)c3cc(-c4cccc(N(c5cccc(-c6ccc7ccccc7c6)c5)c5ccc6oc7c8ccccc8ccc7c6c5)c4)ccc3c3ccccc23)cc1]

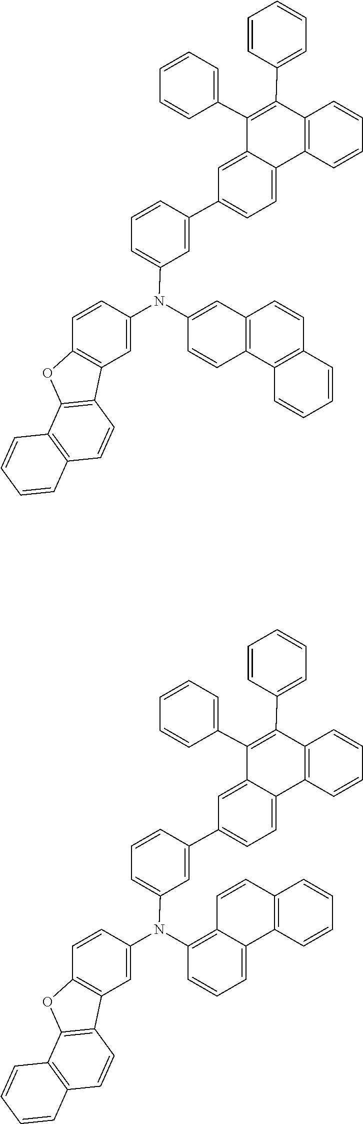 [SMILES: c1ccc(-c2c(-c3ccccc3)c3cc(-c4cccc(N(c5ccc6c(ccc7ccccc76)c5)c5ccc6oc7c8ccccc8ccc7c6c5)c4)ccc3c3ccccc23)cc1 ; c1ccc(-c2c(-c3ccccc3)c3cc(-c4cccc(N(c5ccc6oc7c8ccccc8ccc7c6c5)c5cccc6c5ccc5ccccc56)c4)ccc3c3ccccc23)cc1]

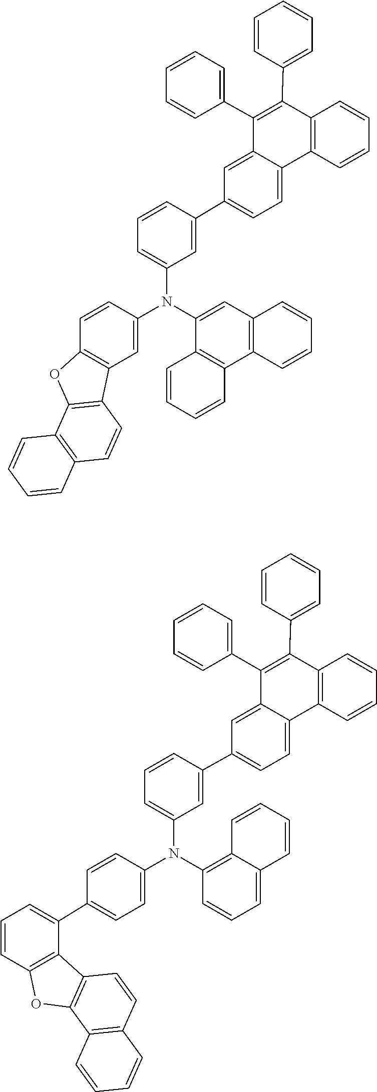 [SMILES: c1ccc(-c2c(-c3ccccc3)c3cc(-c4cccc(N(c5ccc6oc7c8ccccc8ccc7c6c5)c5cc6ccccc6c6ccccc56)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5cccc6ccccc56)c4)ccc3c3ccccc23)cc1]

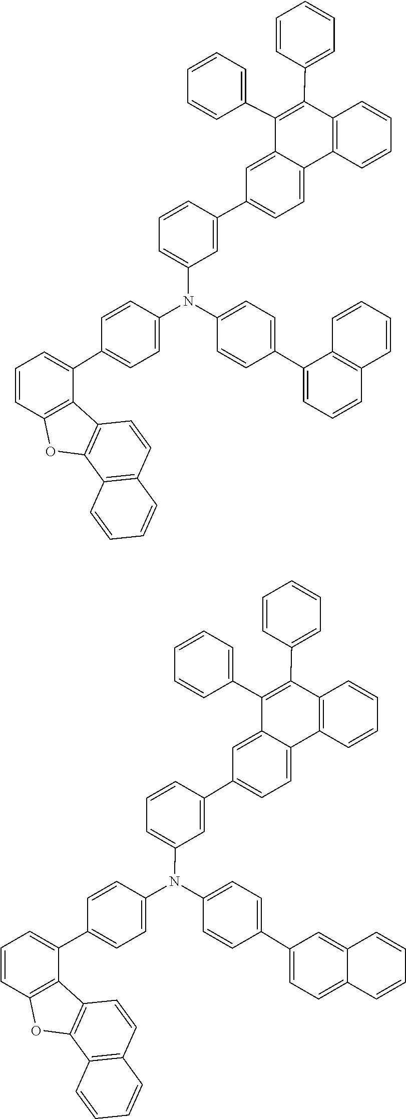 [SMILES: c1ccc(-c2c(-c3ccccc3)c3cc(-c4cccc(N(c5ccc(-c6cccc7ccccc67)cc5)c5ccc(-c6cccc7oc8c9ccccc9ccc8c67)cc5)c4)ccc3c3ccccc23)cc1 ; c1ccc(-c2c(-c3ccccc3)c3cc(-c4cccc(N(c5ccc(-c6ccc7ccccc7c6)cc5)c5ccc(-c6cccc7oc8c9ccccc9ccc8c67)cc5)c4)ccc3c3ccccc23)cc1]

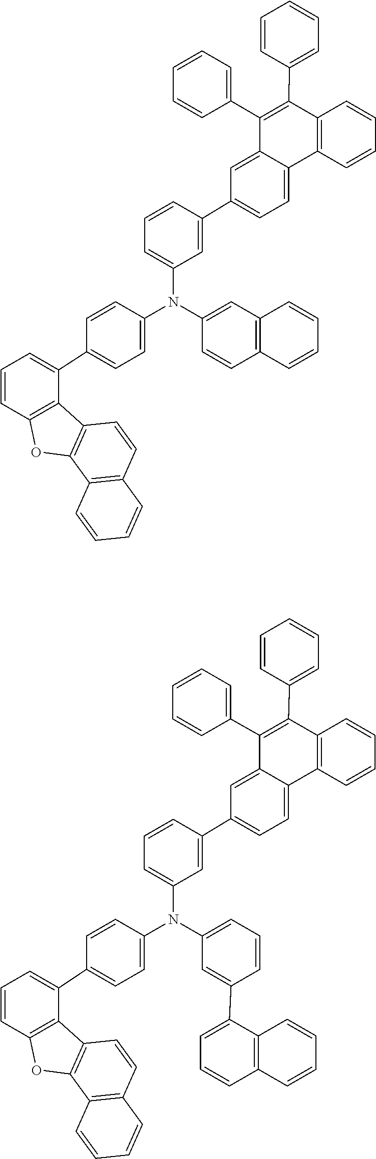 [SMILES: c1ccc(-c2c(-c3ccccc3)c3cc(-c4cccc(N(c5ccc(-c6cccc7oc8c9ccccc9ccc8c67)cc5)c5ccc6ccccc6c5)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5cccc(-c6cccc7ccccc67)c5)c4)ccc3c3ccccc23)cc1]

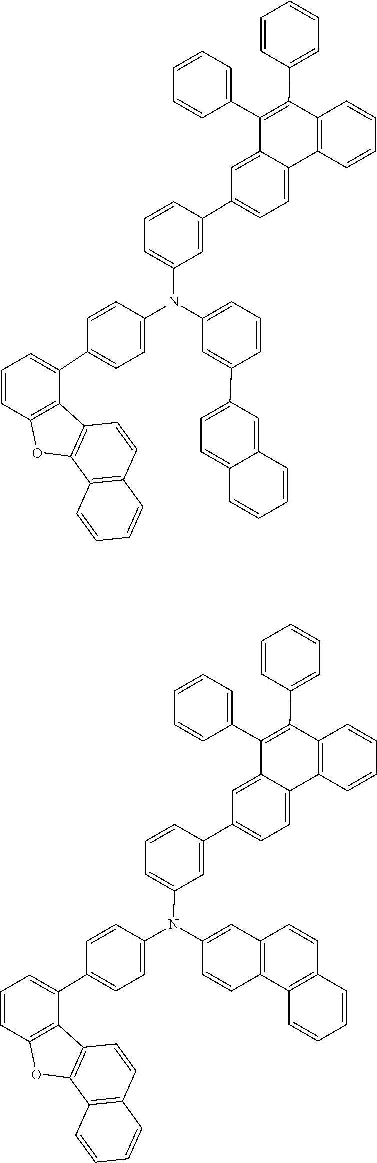 [SMILES: c1ccc(-c2c(-c3ccccc3)c3cc(-c4cccc(N(c5ccc(-c6cccc7oc8c9ccccc9ccc8c67)cc5)c5cccc(-c6ccc7ccccc7c6)c5)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5ccc6c(ccc7ccccc76)c5)c4)ccc3c3ccccc23)cc1]

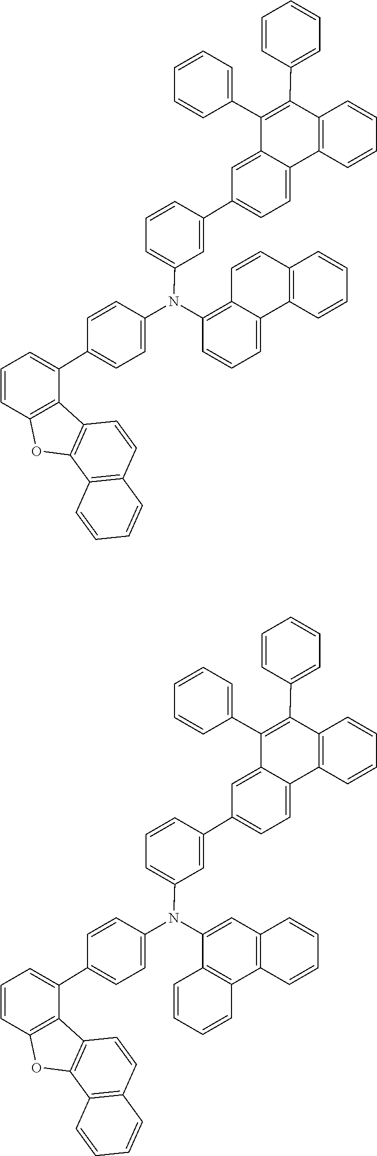 [SMILES: c1ccc(-c2c(-c3ccccc3)c3cc(-c4cccc(N(c5ccc(-c6cccc7oc8c9ccccc9ccc8c67)cc5)c5cccc6c5ccc5ccccc56)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5cc6ccccc6c6ccccc56)c4)ccc3c3ccccc23)cc1]

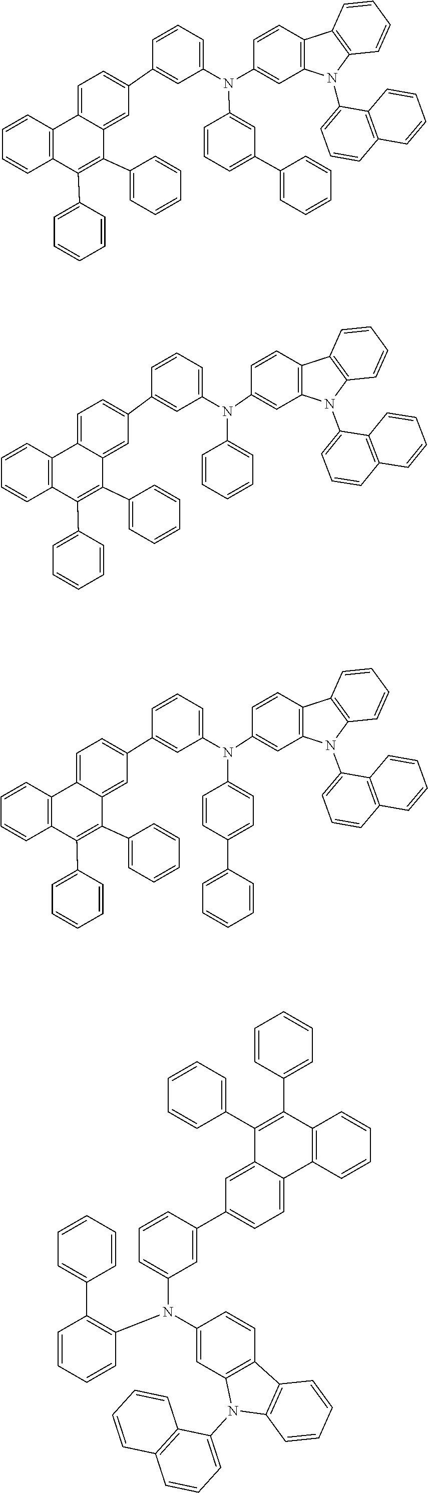 [SMILES: c1ccc(-c2cccc(N(c3cccc(-c4ccc5c(c4)c(-c4ccccc4)c(-c4ccccc4)c4ccccc45)c3)c3ccc4c5ccccc5n(-c5cccc6ccccc56)c4c3)c2)cc1 ; c1ccc(-c2c(-c3ccccc3)c3cc(-c4cccc(N(c5ccccc5)c5ccc6c7ccccc7n(-c7cccc8ccccc78)c6c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3ccc4c5ccccc5n(-c5cccc6ccccc56)c4c3)cc2)cc1 ; c1ccc(-c2ccccc2N(c2cccc(-c3ccc4c(c3)c(-c3ccccc3)c(-c3ccccc3)c3ccccc34)c2)c2ccc3c4ccccc4n(-c4cccc5ccccc45)c3c2)cc1]

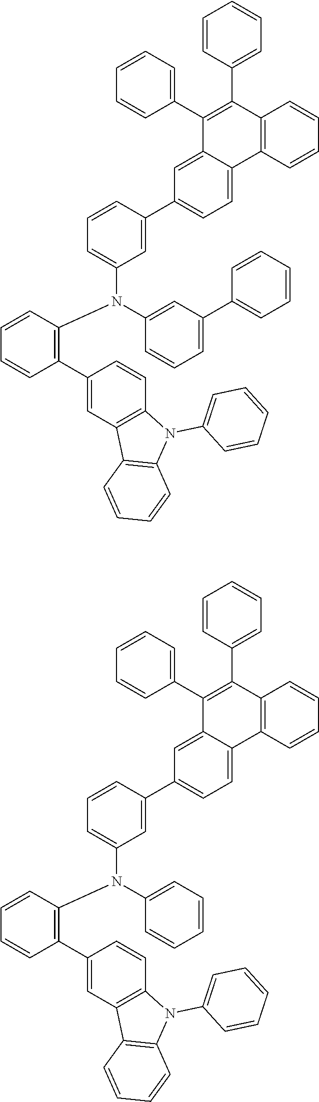 [SMILES: c1ccc(-c2cccc(N(c3cccc(-c4ccc5c(c4)c(-c4ccccc4)c(-c4ccccc4)c4ccccc45)c3)c3ccccc3-c3ccc4c(c3)c3ccccc3n4-c3ccccc3)c2)cc1 ; c1ccc(-c2c(-c3ccccc3)c3cc(-c4cccc(N(c5ccccc5)c5ccccc5-c5ccc6c(c5)c5ccccc5n6-c5ccccc5)c4)ccc3c3ccccc23)cc1]

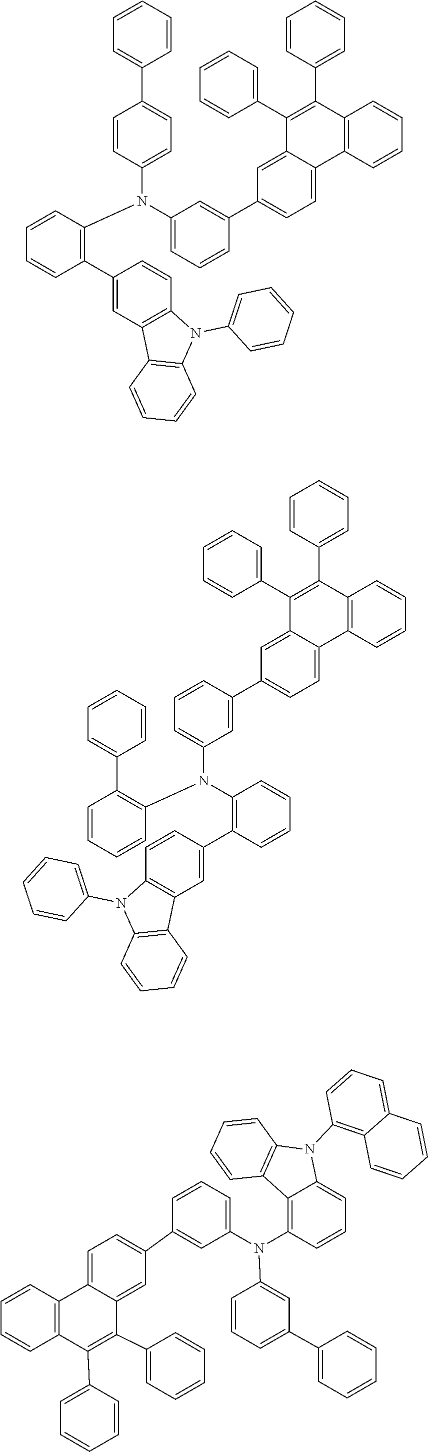 [SMILES: c1ccc(-c2ccc(N(c3cccc(-c4ccc5c(c4)c(-c4ccccc4)c(-c4ccccc4)c4ccccc45)c3)c3ccccc3-c3ccc4c(c3)c3ccccc3n4-c3ccccc3)cc2)cc1 ; c1ccc(-c2ccccc2N(c2cccc(-c3ccc4c(c3)c(-c3ccccc3)c(-c3ccccc3)c3ccccc34)c2)c2ccccc2-c2ccc3c(c2)c2ccccc2n3-c2ccccc2)cc1 ; c1ccc(-c2cccc(N(c3cccc(-c4ccc5c(c4)c(-c4ccccc4)c(-c4ccccc4)c4ccccc45)c3)c3cccc4c3c3ccccc3n4-c3cccc4ccccc34)c2)cc1]

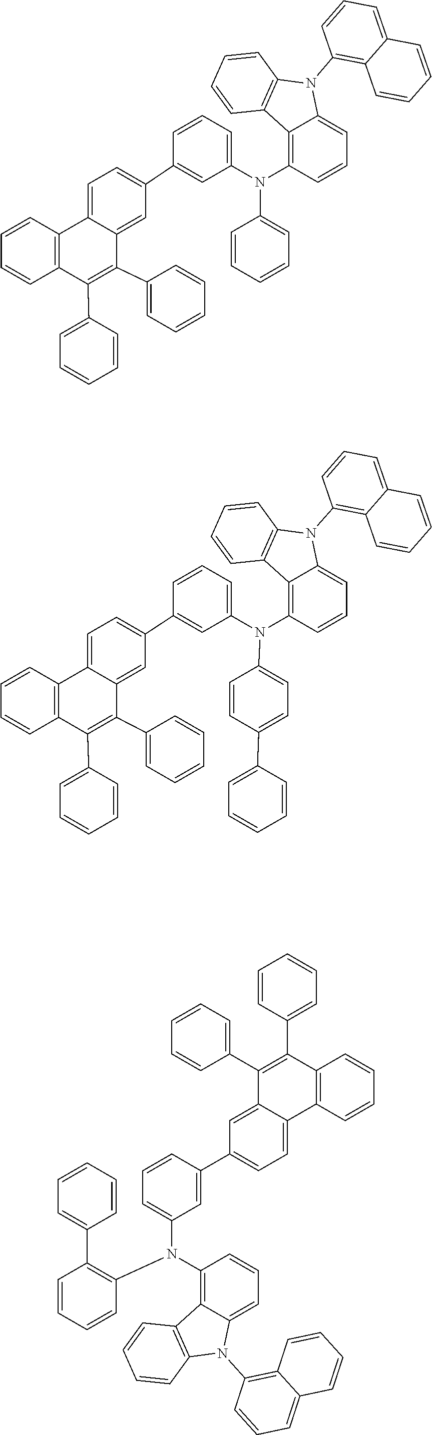 [SMILES: c1ccc(-c2c(-c3ccccc3)c3cc(-c4cccc(N(c5ccccc5)c5cccc6c5c5ccccc5n6-c5cccc6ccccc56)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc4c3c3ccccc3n4-c3cccc4ccccc34)cc2)cc1 ; c1ccc(-c2ccccc2N(c2cccc(-c3ccc4c(c3)c(-c3ccccc3)c(-c3ccccc3)c3ccccc34)c2)c2cccc3c2c2ccccc2n3-c2cccc3ccccc23)cc1]

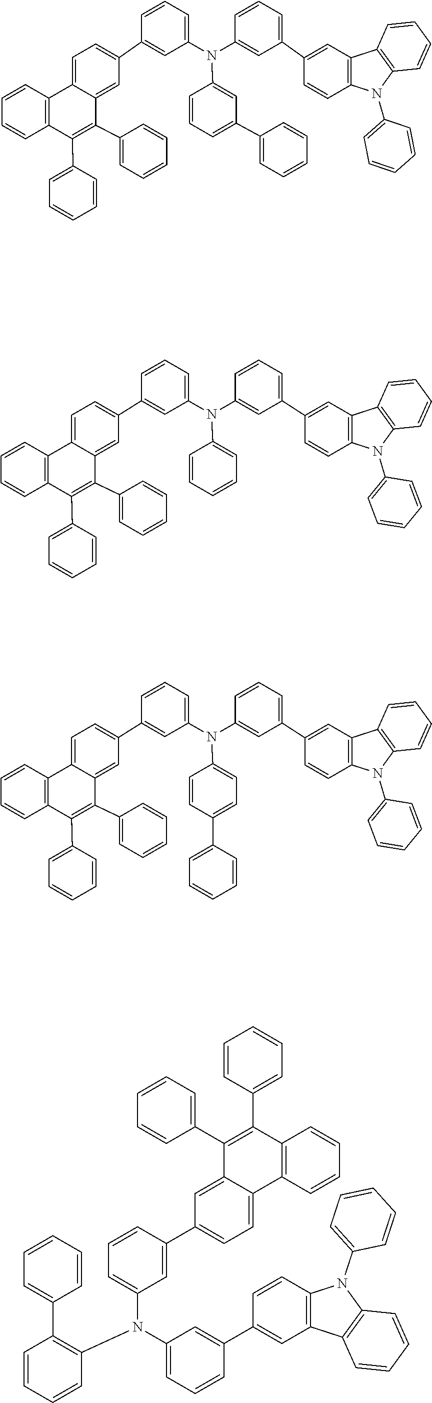 [SMILES: c1ccc(-c2cccc(N(c3cccc(-c4ccc5c(c4)c(-c4ccccc4)c(-c4ccccc4)c4ccccc45)c3)c3cccc(-c4ccc5c(c4)c4ccccc4n5-c4ccccc4)c3)c2)cc1 ; c1ccc(-c2c(-c3ccccc3)c3cc(-c4cccc(N(c5ccccc5)c5cccc(-c6ccc7c(c6)c6ccccc6n7-c6ccccc6)c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc(-c4ccc5c(c4)c4ccccc4n5-c4ccccc4)c3)cc2)cc1 ; c1ccc(-c2ccccc2N(c2cccc(-c3ccc4c(c3)c(-c3ccccc3)c(-c3ccccc3)c3ccccc34)c2)c2cccc(-c3ccc4c(c3)c3ccccc3n4-c3ccccc3)c2)cc1]

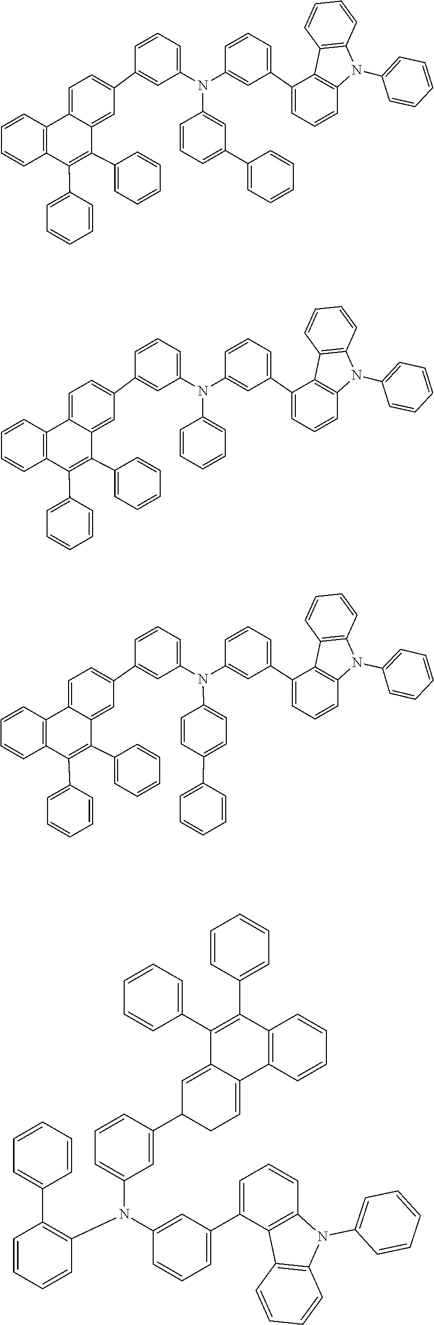 [SMILES: c1ccc(-c2cccc(N(c3cccc(-c4ccc5c(c4)c(-c4ccccc4)c(-c4ccccc4)c4ccccc45)c3)c3cccc(-c4cccc5c4c4ccccc4n5-c4ccccc4)c3)c2)cc1 ; c1ccc(-c2c(-c3ccccc3)c3cc(-c4cccc(N(c5ccccc5)c5cccc(-c6cccc7c6c6ccccc6n7-c6ccccc6)c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc(-c4cccc5c4c4ccccc4n5-c4ccccc4)c3)cc2)cc1 ; C1=c2c(-c3ccccc3)c(-c3ccccc3)c3ccccc3c2=CCC1c1cccc(N(c2cccc(-c3cccc4c3c3ccccc3n4-c3ccccc3)c2)c2ccccc2-c2ccccc2)c1]

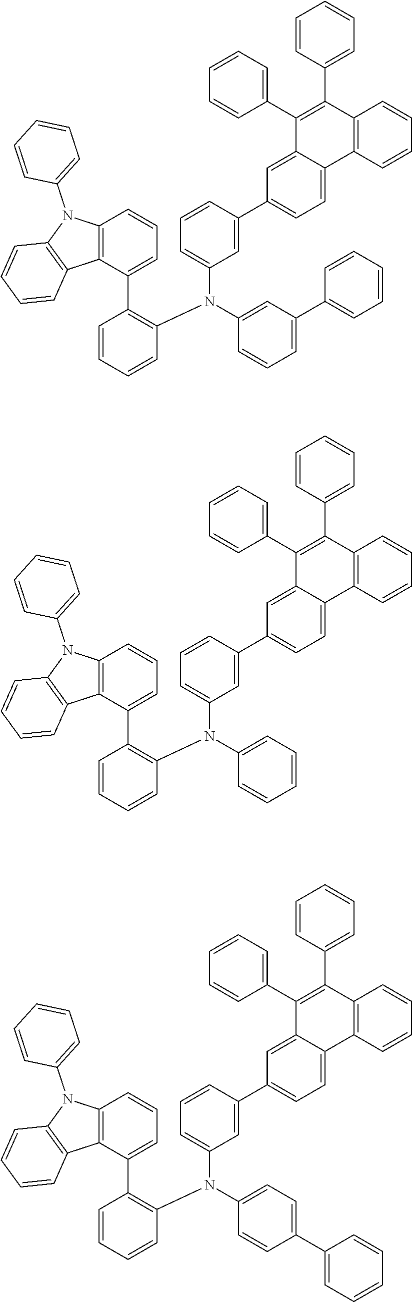 [SMILES: c1ccc(-c2cccc(N(c3cccc(-c4ccc5c(c4)c(-c4ccccc4)c(-c4ccccc4)c4ccccc45)c3)c3ccccc3-c3cccc4c3c3ccccc3n4-c3ccccc3)c2)cc1 ; c1ccc(-c2c(-c3ccccc3)c3cc(-c4cccc(N(c5ccccc5)c5ccccc5-c5cccc6c5c5ccccc5n6-c5ccccc5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3ccccc3-c3cccc4c3c3ccccc3n4-c3ccccc3)cc2)cc1]

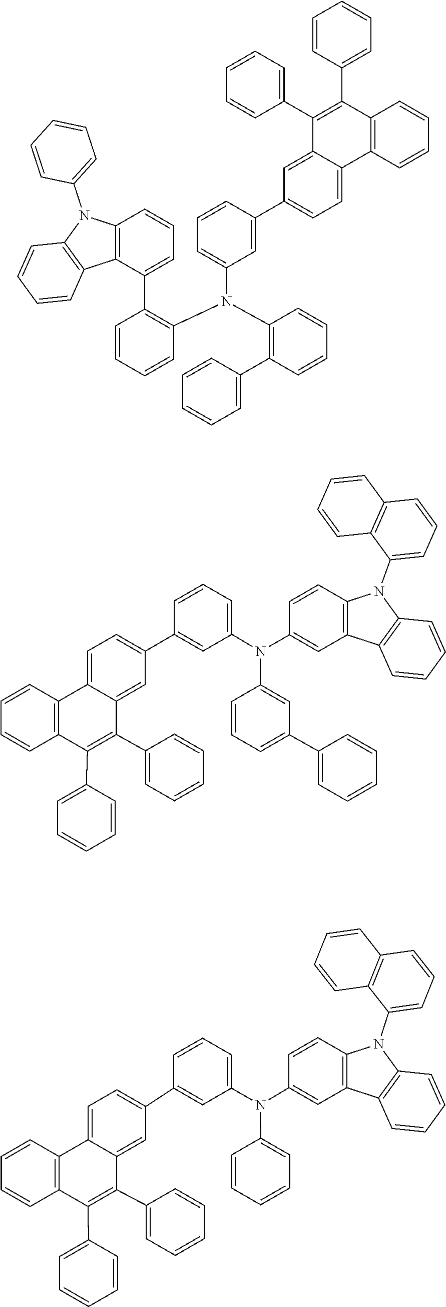 [SMILES: c1ccc(-c2ccccc2N(c2cccc(-c3ccc4c(c3)c(-c3ccccc3)c(-c3ccccc3)c3ccccc34)c2)c2ccccc2-c2cccc3c2c2ccccc2n3-c2ccccc2)cc1 ; c1ccc(-c2cccc(N(c3cccc(-c4ccc5c(c4)c(-c4ccccc4)c(-c4ccccc4)c4ccccc45)c3)c3ccc4c(c3)c3ccccc3n4-c3cccc4ccccc34)c2)cc1 ; c1ccc(-c2c(-c3ccccc3)c3cc(-c4cccc(N(c5ccccc5)c5ccc6c(c5)c5ccccc5n6-c5cccc6ccccc56)c4)ccc3c3ccccc23)cc1]

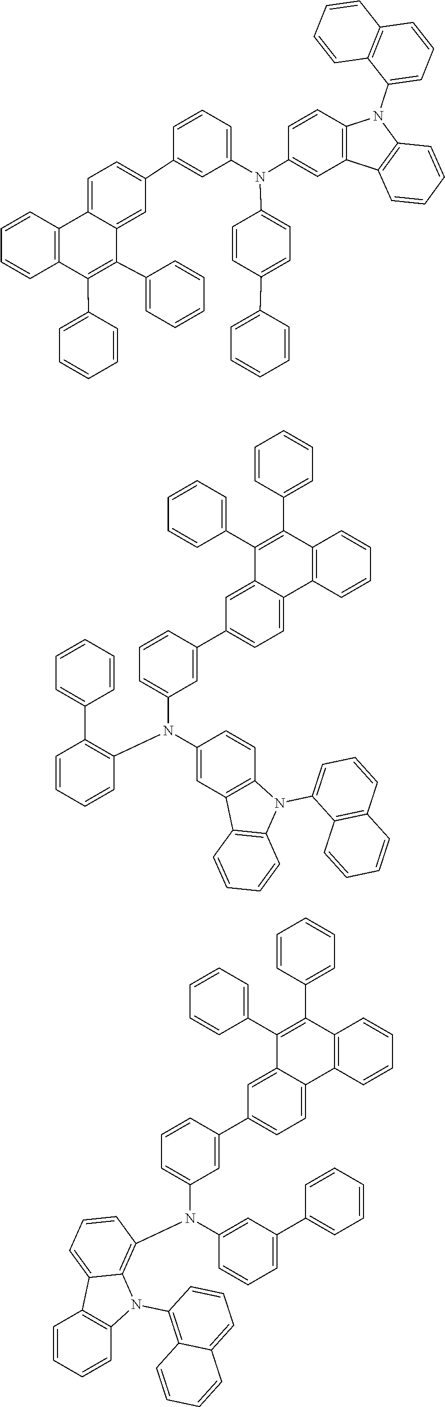 [SMILES: c1ccc(-c2ccc(N(c3cccc(-c4ccc5c(c4)c(-c4ccccc4)c(-c4ccccc4)c4ccccc45)c3)c3ccc4c(c3)c3ccccc3n4-c3cccc4ccccc34)cc2)cc1 ; c1ccc(-c2ccccc2N(c2cccc(-c3ccc4c(c3)c(-c3ccccc3)c(-c3ccccc3)c3ccccc34)c2)c2ccc3c(c2)c2ccccc2n3-c2cccc3ccccc23)cc1 ; c1ccc(-c2cccc(N(c3cccc(-c4ccc5c(c4)c(-c4ccccc4)c(-c4ccccc4)c4ccccc45)c3)c3cccc4c5ccccc5n(-c5cccc6ccccc56)c34)c2)cc1]

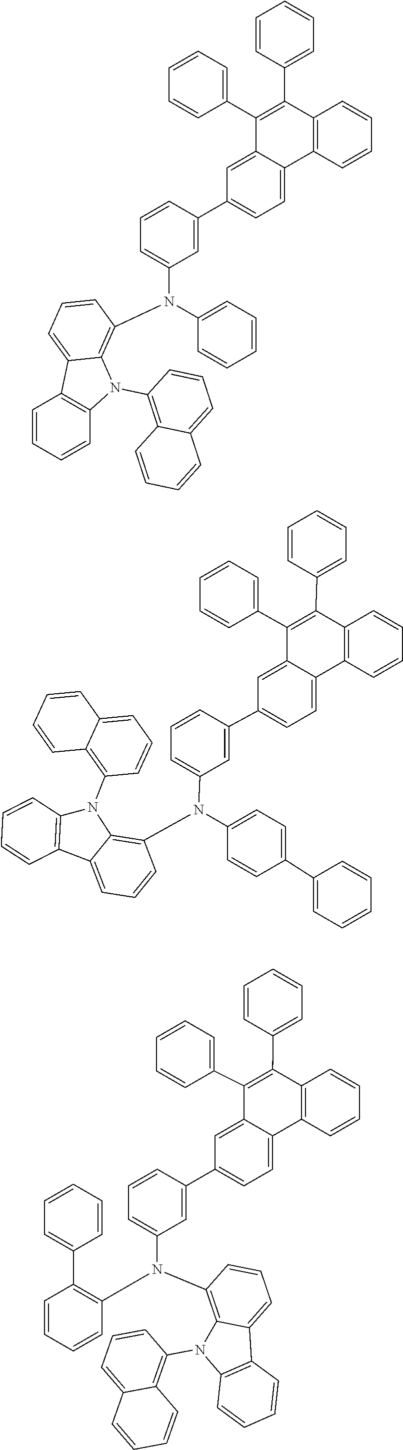

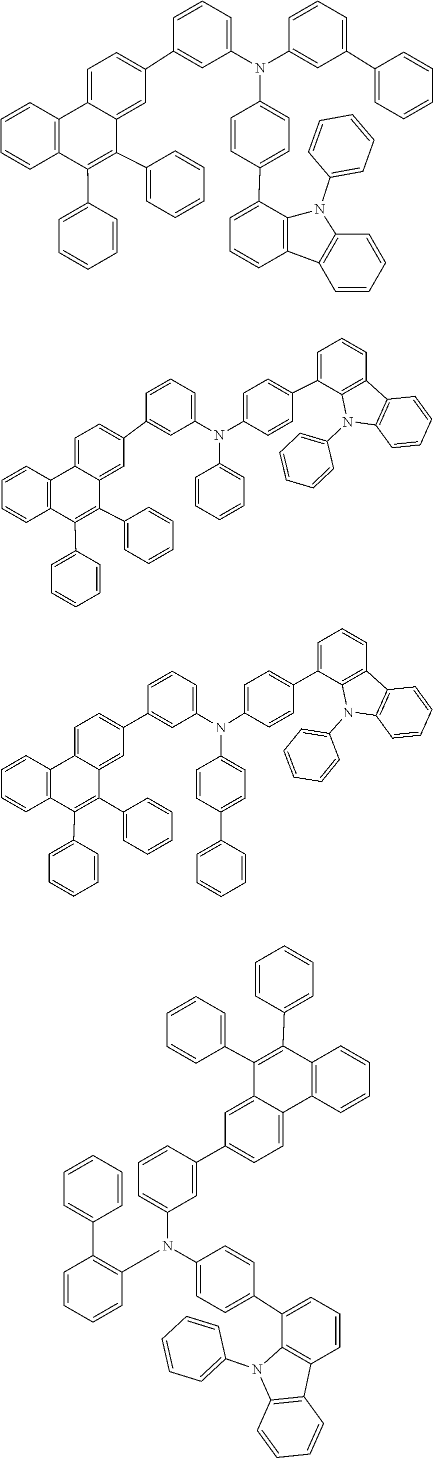

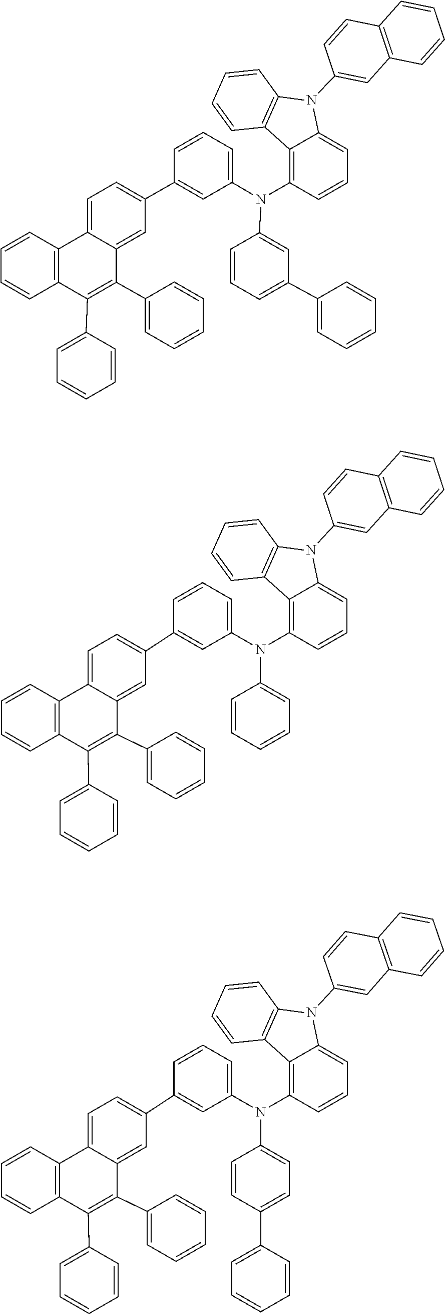 [SMILES: c1ccc(-c2cccc(N(c3cccc(-c4ccc5c(c4)c(-c4ccccc4)c(-c4ccccc4)c4ccccc45)c3)c3cccc4c3c3ccccc3n4-c3ccc4ccccc4c3)c2)cc1 ; c1ccc(-c2c(-c3ccccc3)c3cc(-c4cccc(N(c5ccccc5)c5cccc6c5c5ccccc5n6-c5ccc6ccccc6c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc4c3c3ccccc3n4-c3ccc4ccccc4c3)cc2)cc1]

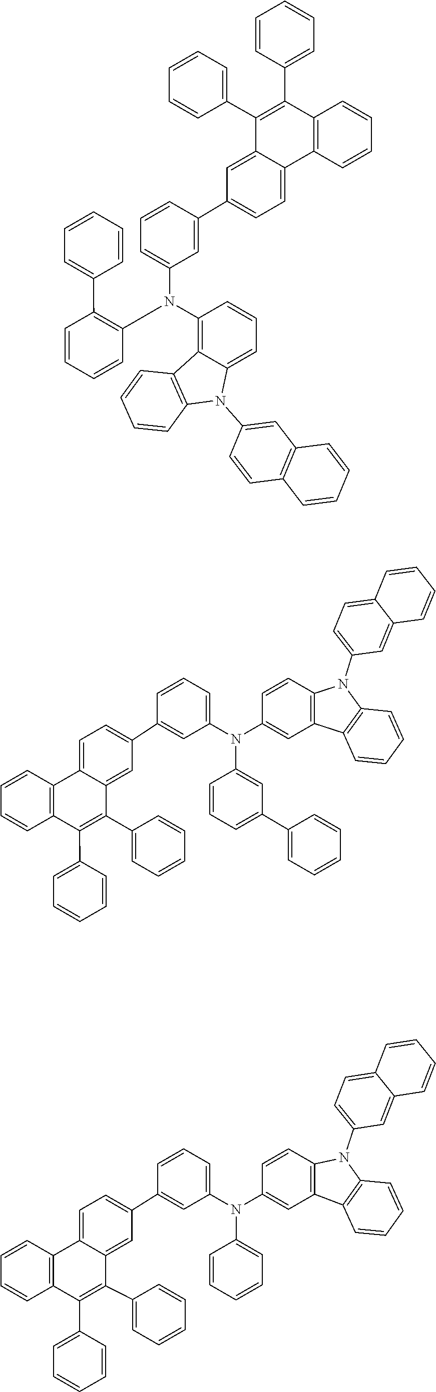

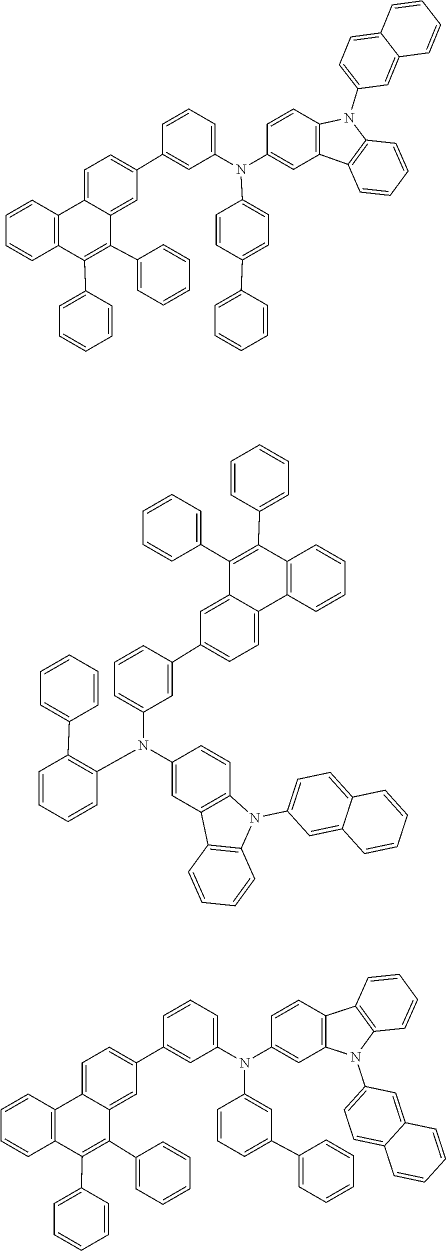

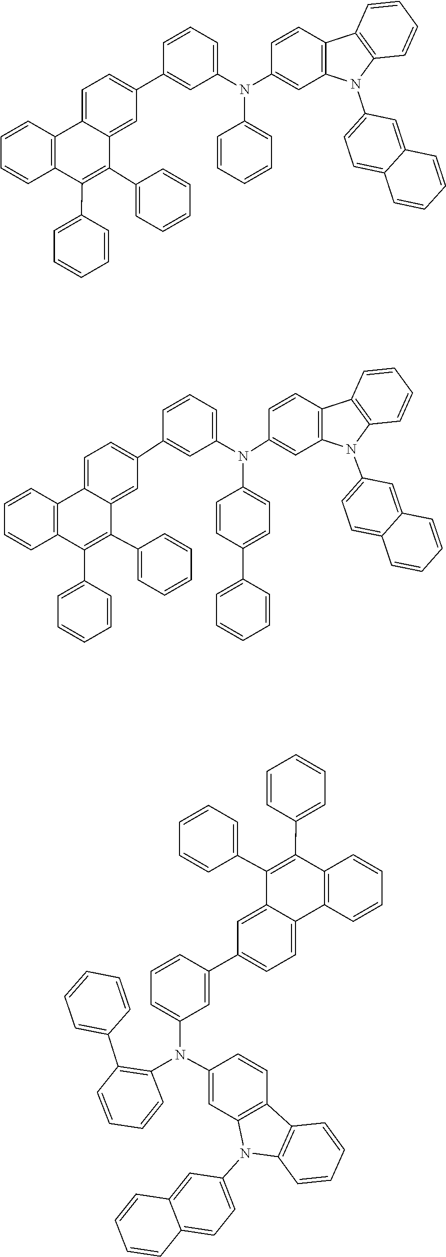 [SMILES: c1ccc(-c2c(-c3ccccc3)c3cc(-c4cccc(N(c5ccccc5)c5ccc6c7ccccc7n(-c7ccc8ccccc8c7)c6c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3ccc4c5ccccc5n(-c5ccc6ccccc6c5)c4c3)cc2)cc1 ; c1ccc(-c2ccccc2N(c2cccc(-c3ccc4c(c3)c(-c3ccccc3)c(-c3ccccc3)c3ccccc34)c2)c2ccc3c4ccccc4n(-c4ccc5ccccc5c4)c3c2)cc1]

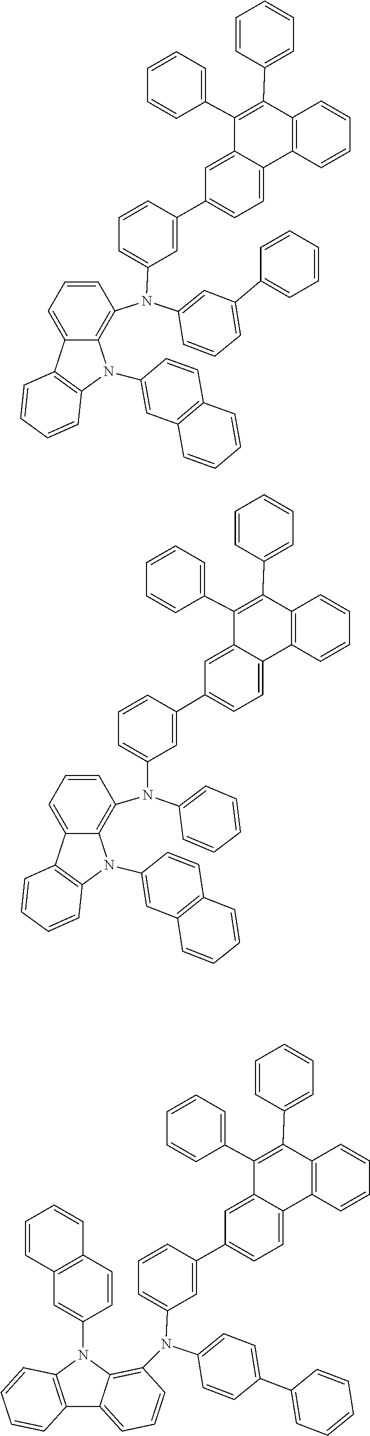 [SMILES: c1ccc(-c2cccc(N(c3cccc(-c4ccc5c(c4)c(-c4ccccc4)c(-c4ccccc4)c4ccccc45)c3)c3cccc4c5ccccc5n(-c5ccc6ccccc6c5)c34)c2)cc1 ; c1ccc(-c2c(-c3ccccc3)c3cc(-c4cccc(N(c5ccccc5)c5cccc6c7ccccc7n(-c7ccc8ccccc8c7)c56)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc4c5ccccc5n(-c5ccc6ccccc6c5)c34)cc2)cc1]

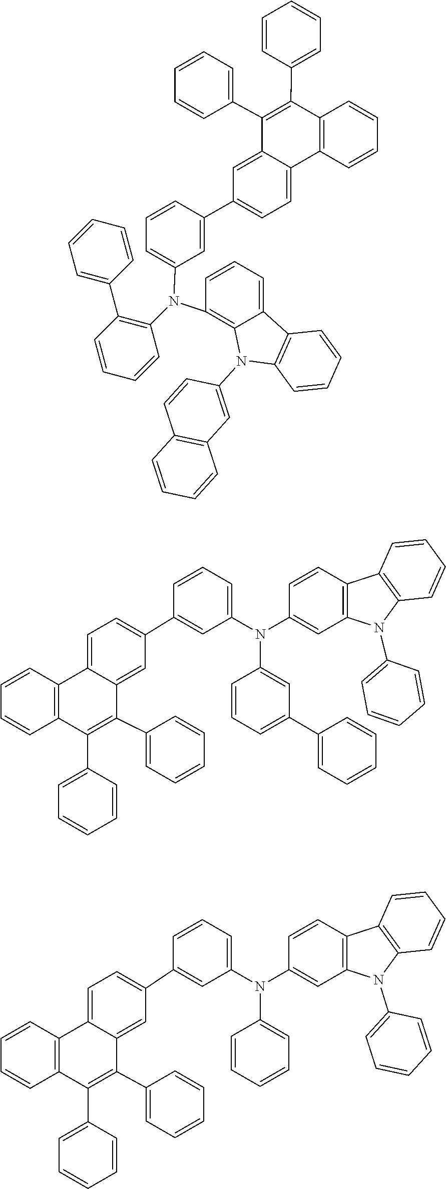 [SMILES: c1ccc(-c2ccccc2N(c2cccc(-c3ccc4c(c3)c(-c3ccccc3)c(-c3ccccc3)c3ccccc34)c2)c2cccc3c4ccccc4n(-c4ccc5ccccc5c4)c23)cc1 ; c1ccc(-c2cccc(N(c3cccc(-c4ccc5c(c4)c(-c4ccccc4)c(-c4ccccc4)c4ccccc45)c3)c3ccc4c5ccccc5n(-c5ccccc5)c4c3)c2)cc1 ; c1ccc(-c2c(-c3ccccc3)c3cc(-c4cccc(N(c5ccccc5)c5ccc6c7ccccc7n(-c7ccccc7)c6c5)c4)ccc3c3ccccc23)cc1]

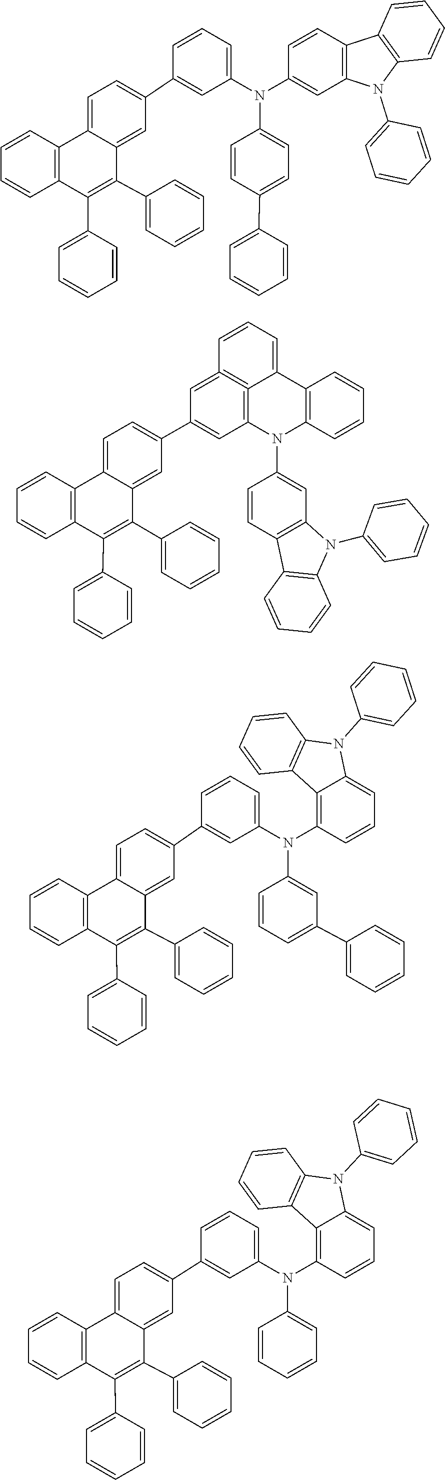

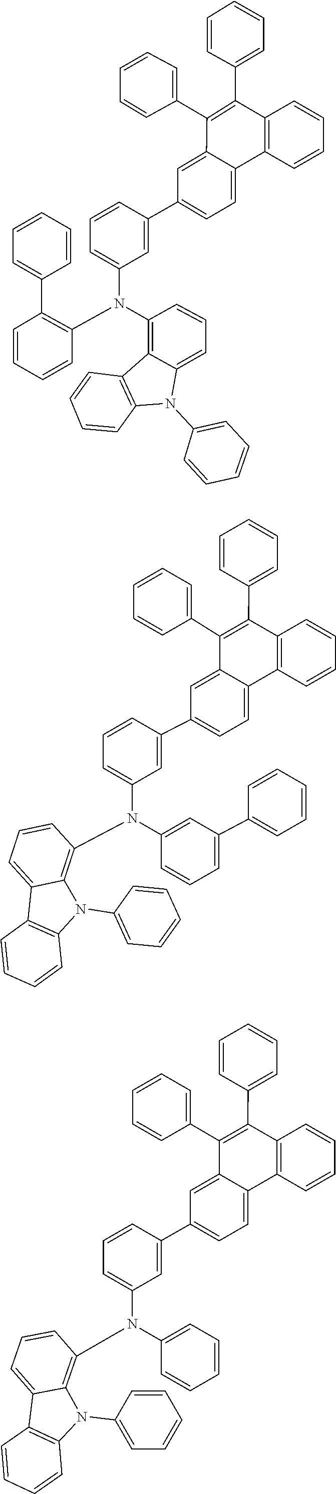

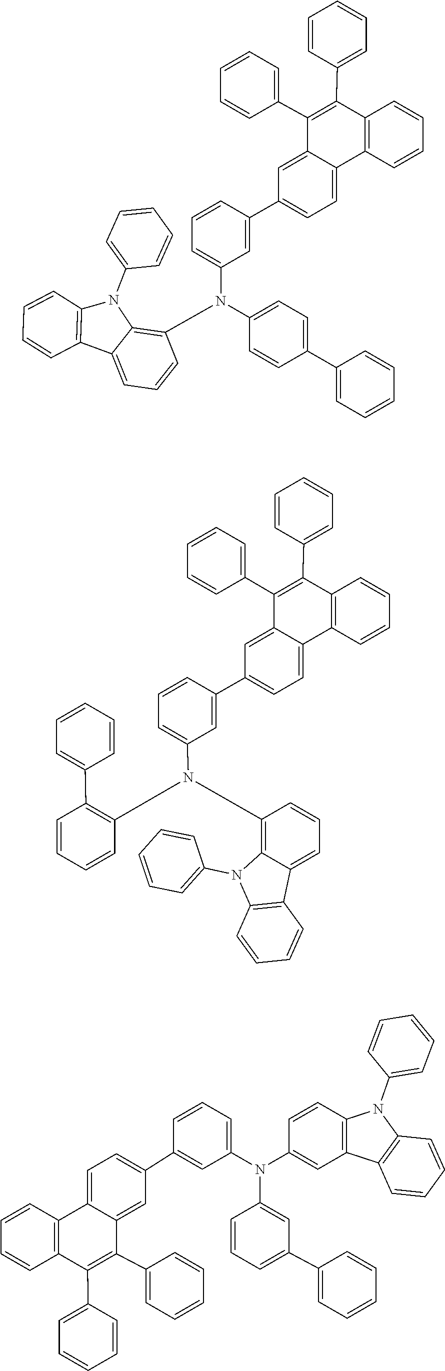 [SMILES: c1ccc(-c2ccc(N(c3cccc(-c4ccc5c(c4)c(-c4ccccc4)c(-c4ccccc4)c4ccccc45)c3)c3cccc4c5ccccc5n(-c5ccccc5)c34)cc2)cc1 ; c1ccc(-c2ccccc2N(c2cccc(-c3ccc4c(c3)c(-c3ccccc3)c(-c3ccccc3)c3ccccc34)c2)c2cccc3c4ccccc4n(-c4ccccc4)c23)cc1 ; c1ccc(-c2cccc(N(c3cccc(-c4ccc5c(c4)c(-c4ccccc4)c(-c4ccccc4)c4ccccc45)c3)c3ccc4c(c3)c3ccccc3n4-c3ccccc3)c2)cc1]

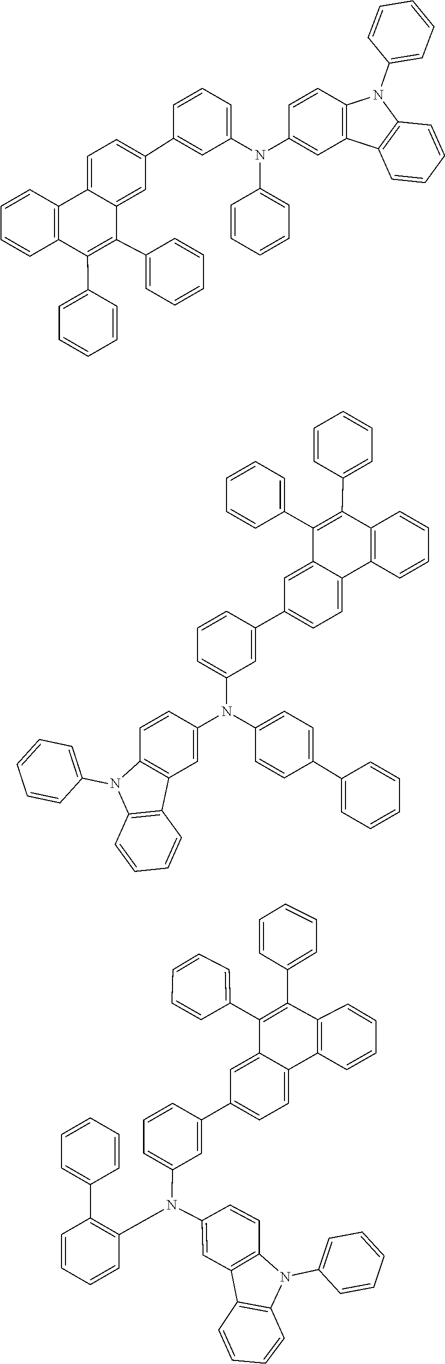 [SMILES: c1ccc(-c2c(-c3ccccc3)c3cc(-c4cccc(N(c5ccccc5)c5ccc6c(c5)c5ccccc5n6-c5ccccc5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3ccc4c(c3)c3ccccc3n4-c3ccccc3)cc2)cc1 ; c1ccc(-c2ccccc2N(c2cccc(-c3ccc4c(c3)c(-c3ccccc3)c(-c3ccccc3)c3ccccc34)c2)c2ccc3c(c2)c2ccccc2n3-c2ccccc2)cc1]

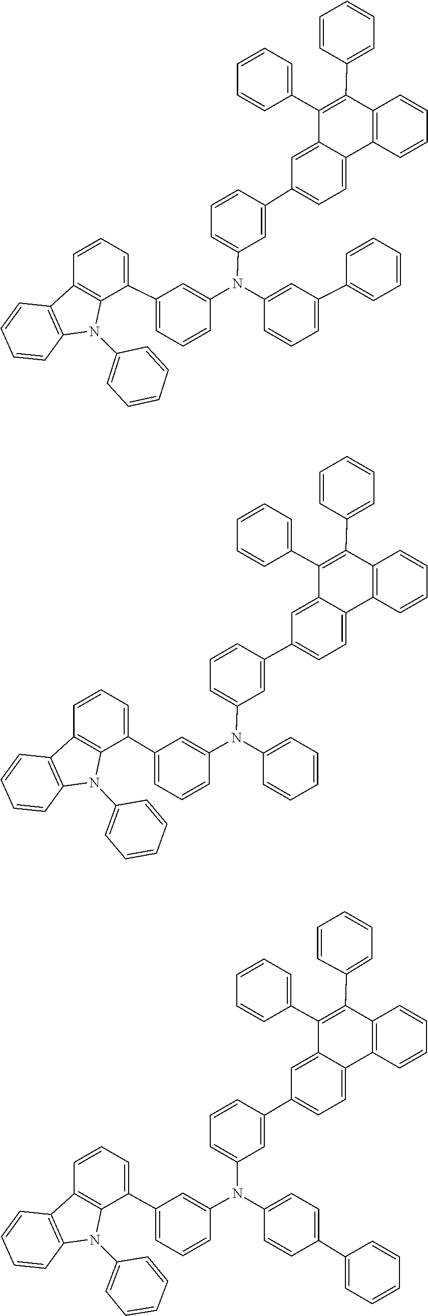 [SMILES: c1ccc(-c2cccc(N(c3cccc(-c4ccc5c(c4)c(-c4ccccc4)c(-c4ccccc4)c4ccccc45)c3)c3cccc(-c4cccc5c6ccccc6n(-c6ccccc6)c45)c3)c2)cc1 ; c1ccc(-c2c(-c3ccccc3)c3cc(-c4cccc(N(c5ccccc5)c5cccc(-c6cccc7c8ccccc8n(-c8ccccc8)c67)c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc(-c4cccc5c6ccccc6n(-c6ccccc6)c45)c3)cc2)cc1]

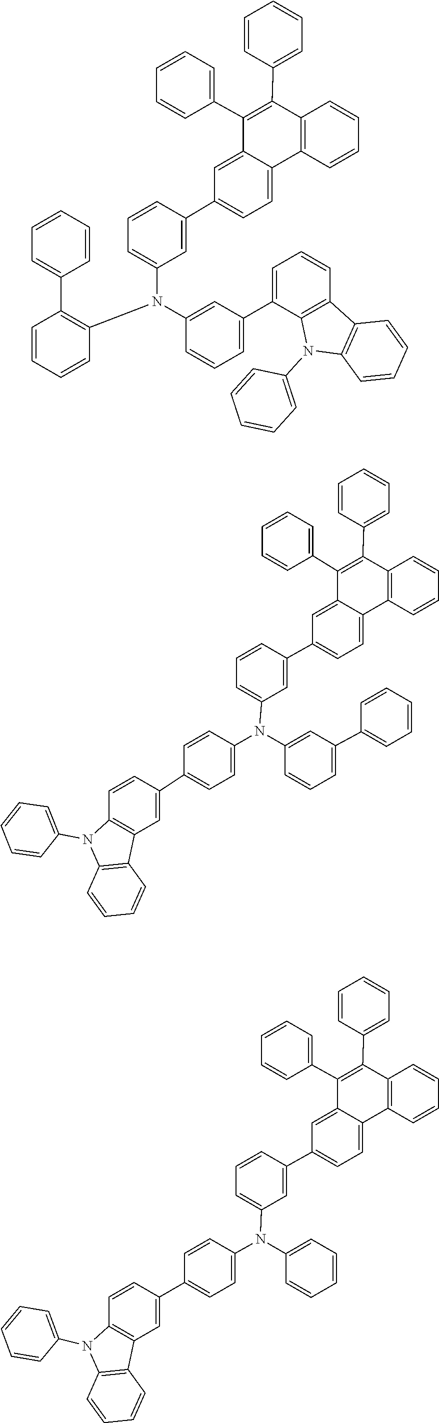 [SMILES: c1ccc(-c2ccccc2N(c2cccc(-c3ccc4c(c3)c(-c3ccccc3)c(-c3ccccc3)c3ccccc34)c2)c2cccc(-c3cccc4c5ccccc5n(-c5ccccc5)c34)c2)cc1 ; c1ccc(-c2cccc(N(c3ccc(-c4ccc5c(c4)c4ccccc4n5-c4ccccc4)cc3)c3cccc(-c4ccc5c(c4)c(-c4ccccc4)c(-c4ccccc4)c4ccccc45)c3)c2)cc1 ; c1ccc(-c2c(-c3ccccc3)c3cc(-c4cccc(N(c5ccccc5)c5ccc(-c6ccc7c(c6)c6ccccc6n7-c6ccccc6)cc5)c4)ccc3c3ccccc23)cc1]

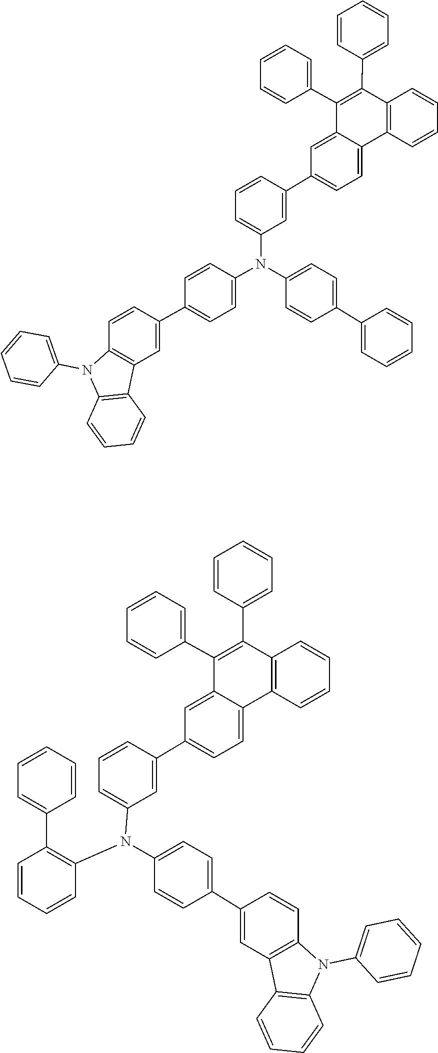 [SMILES: c1ccc(-c2ccc(N(c3ccc(-c4ccc5c(c4)c4ccccc4n5-c4ccccc4)cc3)c3cccc(-c4ccc5c(c4)c(-c4ccccc4)c(-c4ccccc4)c4ccccc45)c3)cc2)cc1 ; c1ccc(-c2ccccc2N(c2ccc(-c3ccc4c(c3)c3ccccc3n4-c3ccccc3)cc2)c2cccc(-c3ccc4c(c3)c(-c3ccccc3)c(-c3ccccc3)c3ccccc34)c2)cc1]

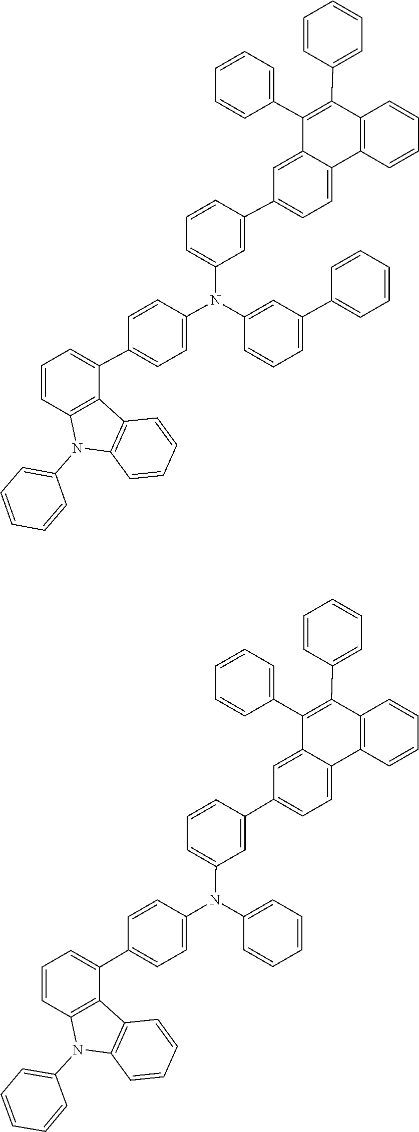 [SMILES: c1ccc(-c2cccc(N(c3ccc(-c4cccc5c4c4ccccc4n5-c4ccccc4)cc3)c3cccc(-c4ccc5c(c4)c(-c4ccccc4)c(-c4ccccc4)c4ccccc45)c3)c2)cc1 ; c1ccc(-c2c(-c3ccccc3)c3cc(-c4cccc(N(c5ccccc5)c5ccc(-c6cccc7c6c6ccccc6n7-c6ccccc6)cc5)c4)ccc3c3ccccc23)cc1]

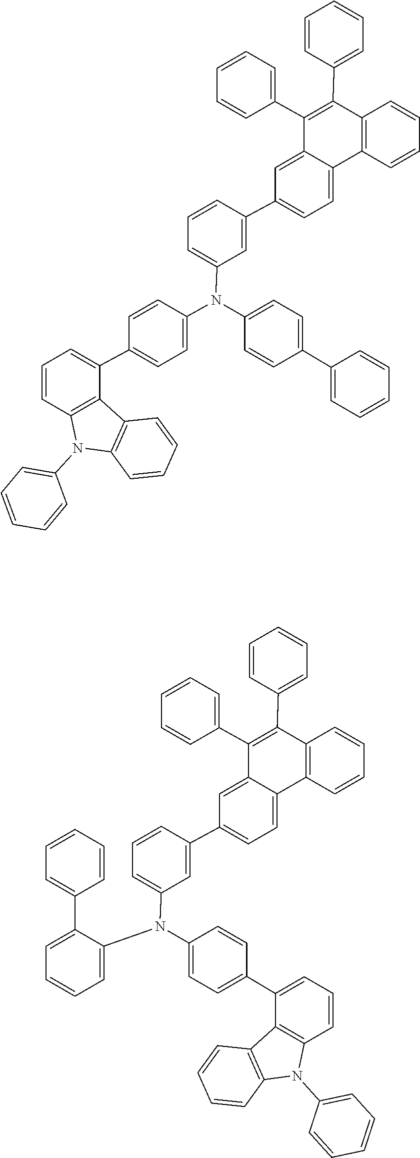 [SMILES: c1ccc(-c2ccc(N(c3ccc(-c4cccc5c4c4ccccc4n5-c4ccccc4)cc3)c3cccc(-c4ccc5c(c4)c(-c4ccccc4)c(-c4ccccc4)c4ccccc45)c3)cc2)cc1 ; c1ccc(-c2ccccc2N(c2ccc(-c3cccc4c3c3ccccc3n4-c3ccccc3)cc2)c2cccc(-c3ccc4c(c3)c(-c3ccccc3)c(-c3ccccc3)c3ccccc34)c2)cc1]

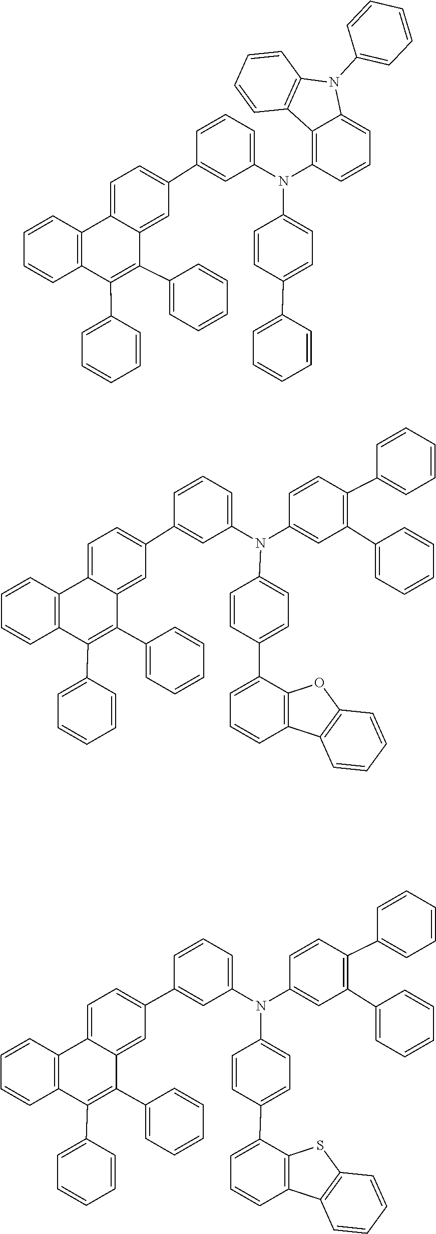 [SMILES: c1ccc(-c2ccc(N(c3cccc(-c4ccc5c(c4)c(-c4ccccc4)c(-c4ccccc4)c4ccccc45)c3)c3cccc4c3c3ccccc3n4-c3ccccc3)cc2)cc1 ; c1ccc(-c2ccc(N(c3ccc(-c4cccc5c4oc4ccccc45)cc3)c3cccc(-c4ccc5c(c4)c(-c4ccccc4)c(-c4ccccc4)c4ccccc45)c3)cc2-c2ccccc2)cc1 ; c1ccc(-c2ccc(N(c3ccc(-c4cccc5c4sc4ccccc45)cc3)c3cccc(-c4ccc5c(c4)c(-c4ccccc4)c(-c4ccccc4)c4ccccc45)c3)cc2-c2ccccc2)cc1]

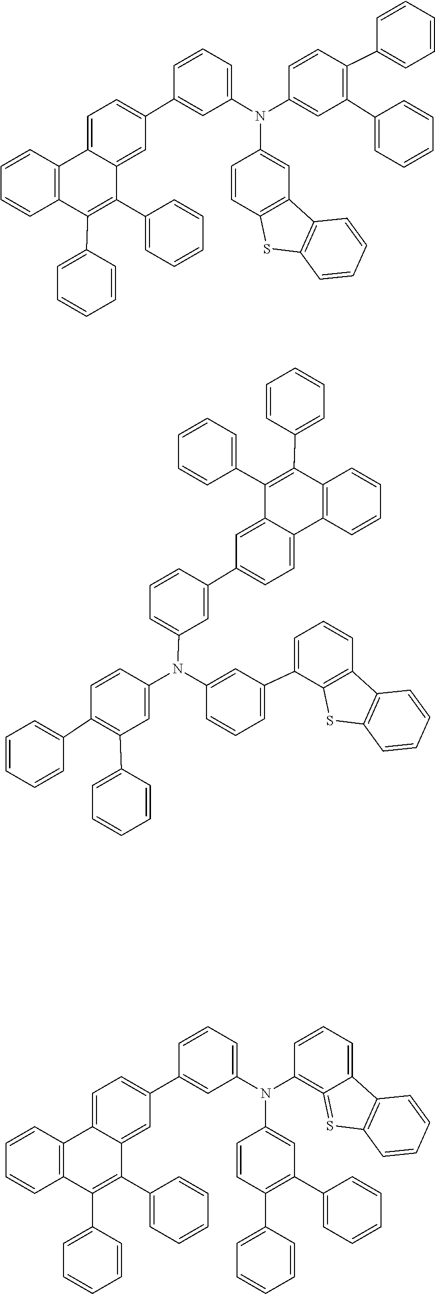

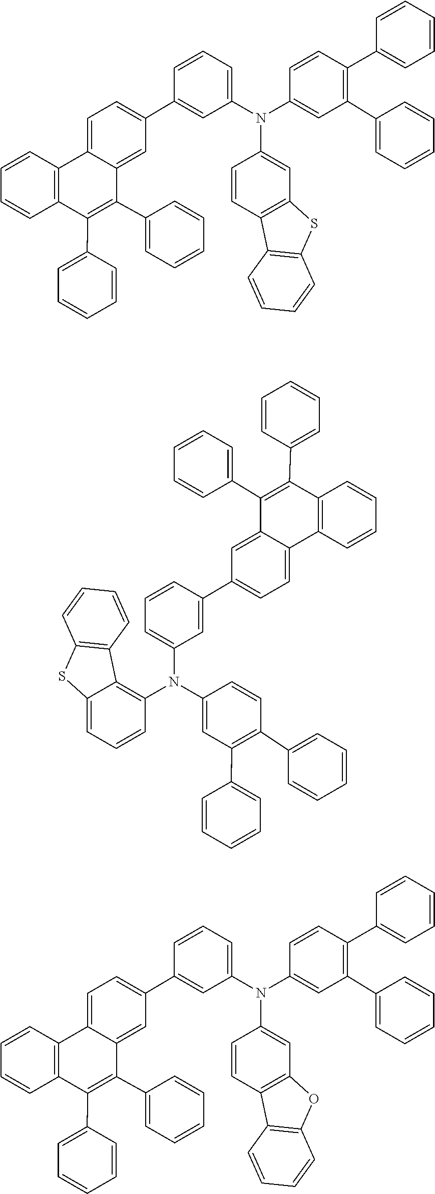 [SMILES: c1ccc(-c2ccc(N(c3cccc(-c4ccc5c(c4)c(-c4ccccc4)c(-c4ccccc4)c4ccccc45)c3)c3ccc4c(c3)sc3ccccc34)cc2-c2ccccc2)cc1 ; c1ccc(-c2ccc(N(c3cccc(-c4ccc5c(c4)c(-c4ccccc4)c(-c4ccccc4)c4ccccc45)c3)c3cccc4sc5ccccc5c34)cc2-c2ccccc2)cc1 ; c1ccc(-c2ccc(N(c3cccc(-c4ccc5c(c4)c(-c4ccccc4)c(-c4ccccc4)c4ccccc45)c3)c3ccc4c(c3)oc3ccccc34)cc2-c2ccccc2)cc1]

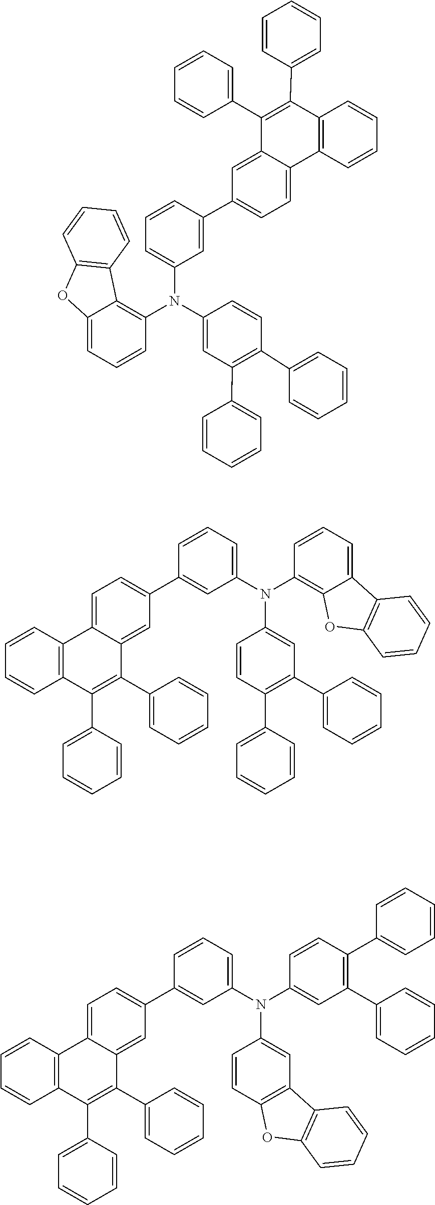 [SMILES: c1ccc(-c2ccc(N(c3cccc(-c4ccc5c(c4)c(-c4ccccc4)c(-c4ccccc4)c4ccccc45)c3)c3cccc4oc5ccccc5c34)cc2-c2ccccc2)cc1 ; c1ccc(-c2ccc(N(c3cccc(-c4ccc5c(c4)c(-c4ccccc4)c(-c4ccccc4)c4ccccc45)c3)c3cccc4c3oc3ccccc34)cc2-c2ccccc2)cc1 ; c1ccc(-c2ccc(N(c3cccc(-c4ccc5c(c4)c(-c4ccccc4)c(-c4ccccc4)c4ccccc45)c3)c3ccc4oc5ccccc5c4c3)cc2-c2ccccc2)cc1]

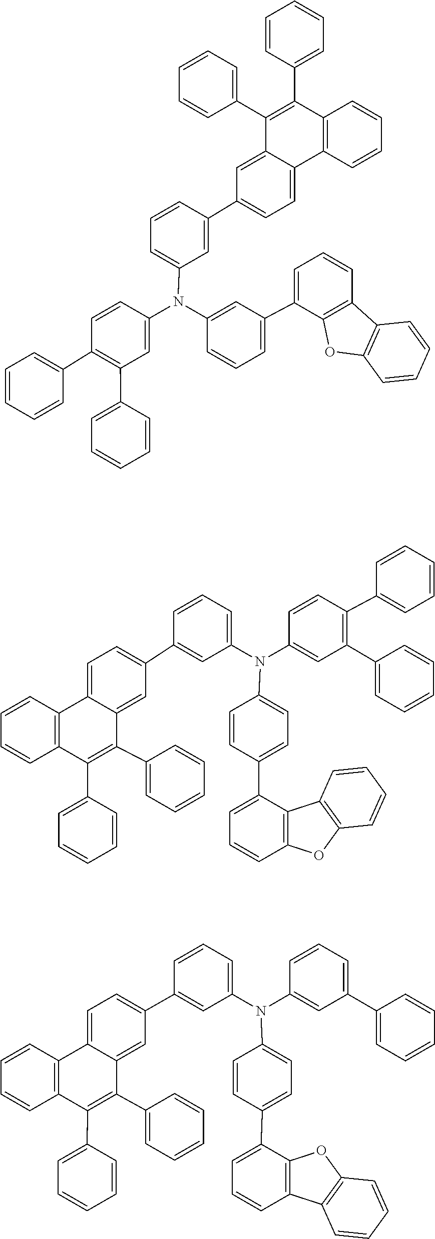 [SMILES: c1ccc(-c2ccc(N(c3cccc(-c4ccc5c(c4)c(-c4ccccc4)c(-c4ccccc4)c4ccccc45)c3)c3cccc(-c4cccc5c4oc4ccccc45)c3)cc2-c2ccccc2)cc1 ; c1ccc(-c2ccc(N(c3ccc(-c4cccc5oc6ccccc6c45)cc3)c3cccc(-c4ccc5c(c4)c(-c4ccccc4)c(-c4ccccc4)c4ccccc45)c3)cc2-c2ccccc2)cc1 ; c1ccc(-c2cccc(N(c3ccc(-c4cccc5c4oc4ccccc45)cc3)c3cccc(-c4ccc5c(c4)c(-c4ccccc4)c(-c4ccccc4)c4ccccc45)c3)c2)cc1]

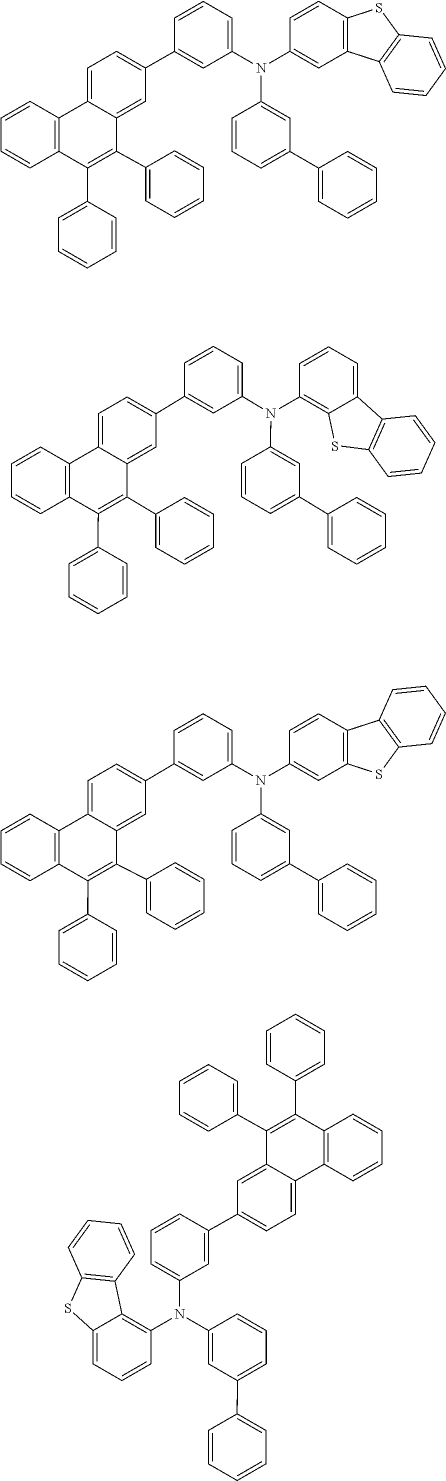 [SMILES: c1ccc(-c2cccc(N(c3cccc(-c4ccc5c(c4)c(-c4ccccc4)c(-c4ccccc4)c4ccccc45)c3)c3ccc4sc5ccccc5c4c3)c2)cc1 ; c1ccc(-c2cccc(N(c3cccc(-c4ccc5c(c4)c(-c4ccccc4)c(-c4ccccc4)c4ccccc45)c3)c3cccc4c3sc3ccccc34)c2)cc1 ; c1ccc(-c2cccc(N(c3cccc(-c4ccc5c(c4)c(-c4ccccc4)c(-c4ccccc4)c4ccccc45)c3)c3ccc4c(c3)sc3ccccc34)c2)cc1 ; c1ccc(-c2cccc(N(c3cccc(-c4ccc5c(c4)c(-c4ccccc4)c(-c4ccccc4)c4ccccc45)c3)c3cccc4sc5ccccc5c34)c2)cc1]

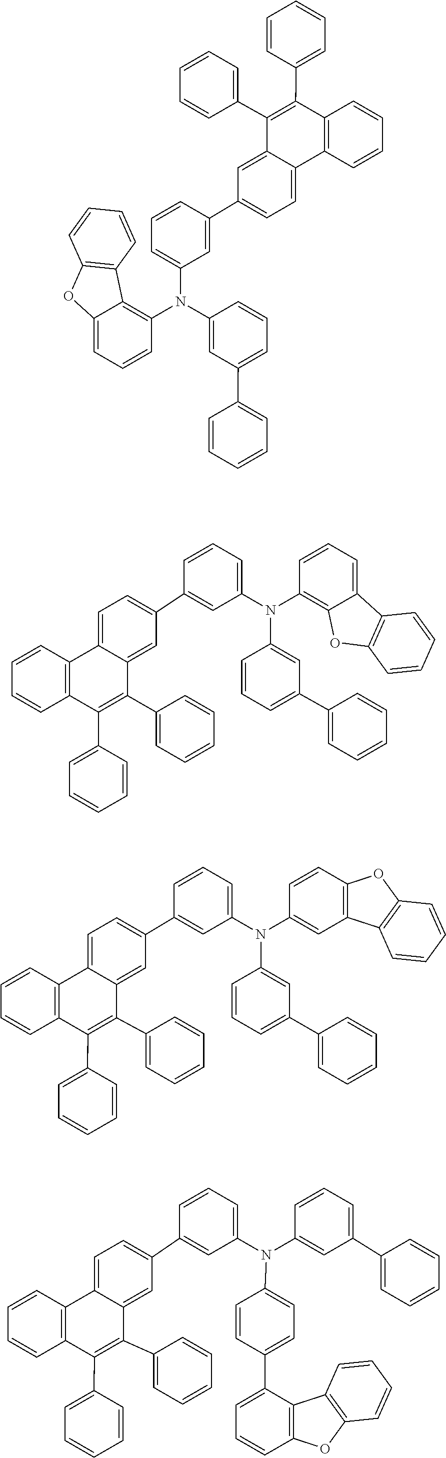 [SMILES: c1ccc(-c2cccc(N(c3cccc(-c4ccc5c(c4)c(-c4ccccc4)c(-c4ccccc4)c4ccccc45)c3)c3cccc4oc5ccccc5c34)c2)cc1 ; c1ccc(-c2cccc(N(c3cccc(-c4ccc5c(c4)c(-c4ccccc4)c(-c4ccccc4)c4ccccc45)c3)c3cccc4c3oc3ccccc34)c2)cc1 ; c1ccc(-c2cccc(N(c3cccc(-c4ccc5c(c4)c(-c4ccccc4)c(-c4ccccc4)c4ccccc45)c3)c3ccc4oc5ccccc5c4c3)c2)cc1 ; c1ccc(-c2cccc(N(c3ccc(-c4cccc5oc6ccccc6c45)cc3)c3cccc(-c4ccc5c(c4)c(-c4ccccc4)c(-c4ccccc4)c4ccccc45)c3)c2)cc1]

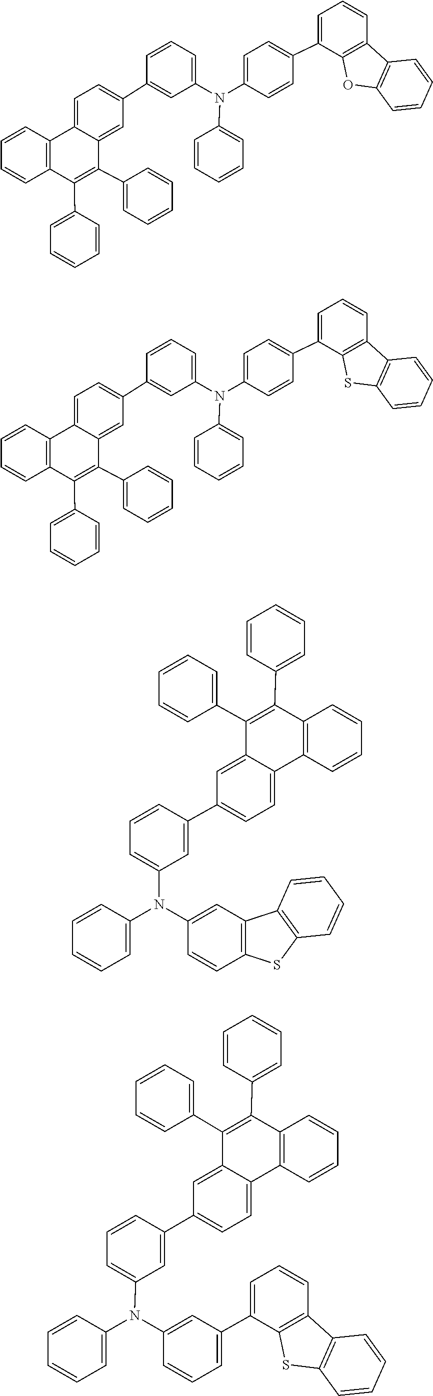 [SMILES: c1ccc(-c2c(-c3ccccc3)c3cc(-c4cccc(N(c5ccccc5)c5ccc(-c6cccc7c6oc6ccccc67)cc5)c4)ccc3c3ccccc23)cc1 ; c1ccc(-c2c(-c3ccccc3)c3cc(-c4cccc(N(c5ccccc5)c5ccc(-c6cccc7c6sc6ccccc67)cc5)c4)ccc3c3ccccc23)cc1 ; c1ccc(-c2c(-c3ccccc3)c3cc(-c4cccc(N(c5ccccc5)c5ccc6sc7ccccc7c6c5)c4)ccc3c3ccccc23)cc1 ; c1ccc(-c2c(-c3ccccc3)c3cc(-c4cccc(N(c5ccccc5)c5cccc(-c6cccc7c6sc6ccccc67)c5)c4)ccc3c3ccccc23)cc1]

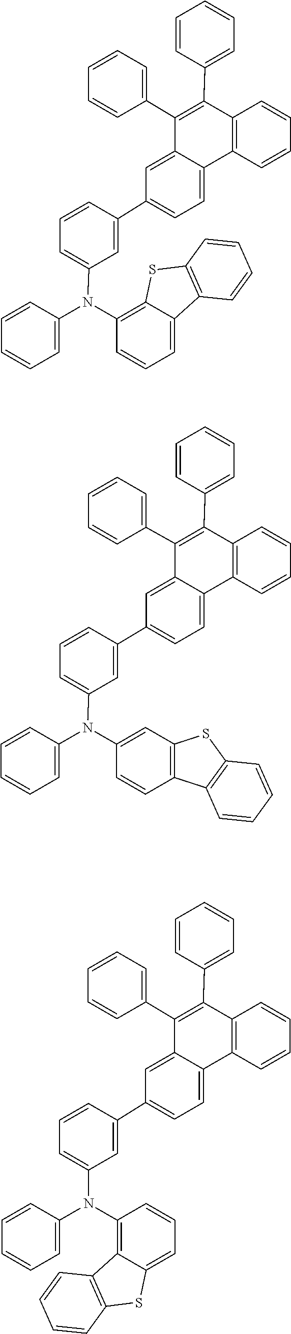 [SMILES: c1ccc(-c2c(-c3ccccc3)c3cc(-c4cccc(N(c5ccccc5)c5cccc6c5sc5ccccc56)c4)ccc3c3ccccc23)cc1 ; c1ccc(-c2c(-c3ccccc3)c3cc(-c4cccc(N(c5ccccc5)c5ccc6c(c5)sc5ccccc56)c4)ccc3c3ccccc23)cc1 ; c1ccc(-c2c(-c3ccccc3)c3cc(-c4cccc(N(c5ccccc5)c5cccc6sc7ccccc7c56)c4)ccc3c3ccccc23)cc1]

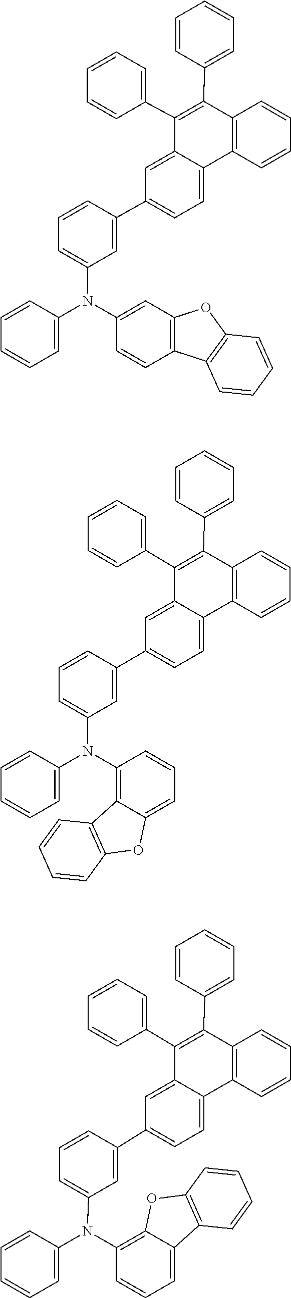 [SMILES: c1ccc(-c2c(-c3ccccc3)c3cc(-c4cccc(N(c5ccccc5)c5ccc6c(c5)oc5ccccc56)c4)ccc3c3ccccc23)cc1 ; c1ccc(-c2c(-c3ccccc3)c3cc(-c4cccc(N(c5ccccc5)c5cccc6oc7ccccc7c56)c4)ccc3c3ccccc23)cc1 ; c1ccc(-c2c(-c3ccccc3)c3cc(-c4cccc(N(c5ccccc5)c5cccc6c5oc5ccccc56)c4)ccc3c3ccccc23)cc1]

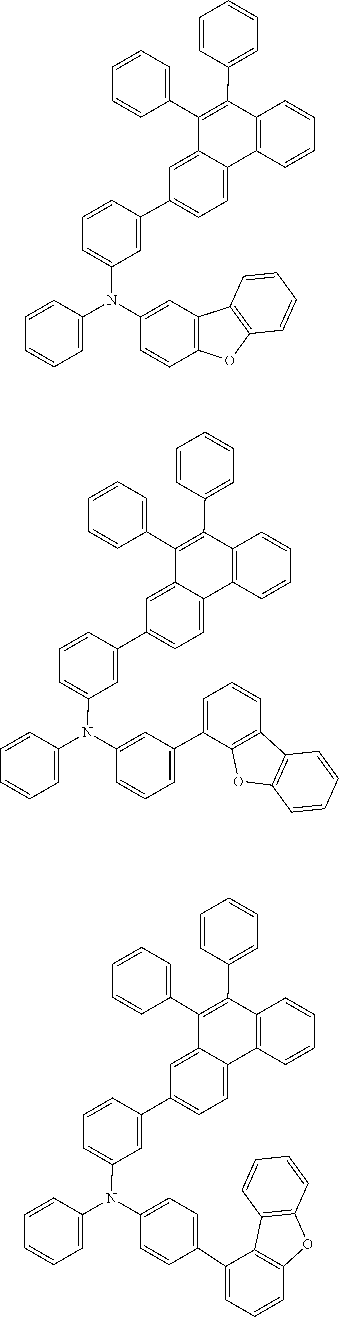 [SMILES: c1ccc(-c2c(-c3ccccc3)c3cc(-c4cccc(N(c5ccccc5)c5ccc6oc7ccccc7c6c5)c4)ccc3c3ccccc23)cc1 ; c1ccc(-c2c(-c3ccccc3)c3cc(-c4cccc(N(c5ccccc5)c5cccc(-c6cccc7c6oc6ccccc67)c5)c4)ccc3c3ccccc23)cc1 ; c1ccc(-c2c(-c3ccccc3)c3cc(-c4cccc(N(c5ccccc5)c5ccc(-c6cccc7oc8ccccc8c67)cc5)c4)ccc3c3ccccc23)cc1]

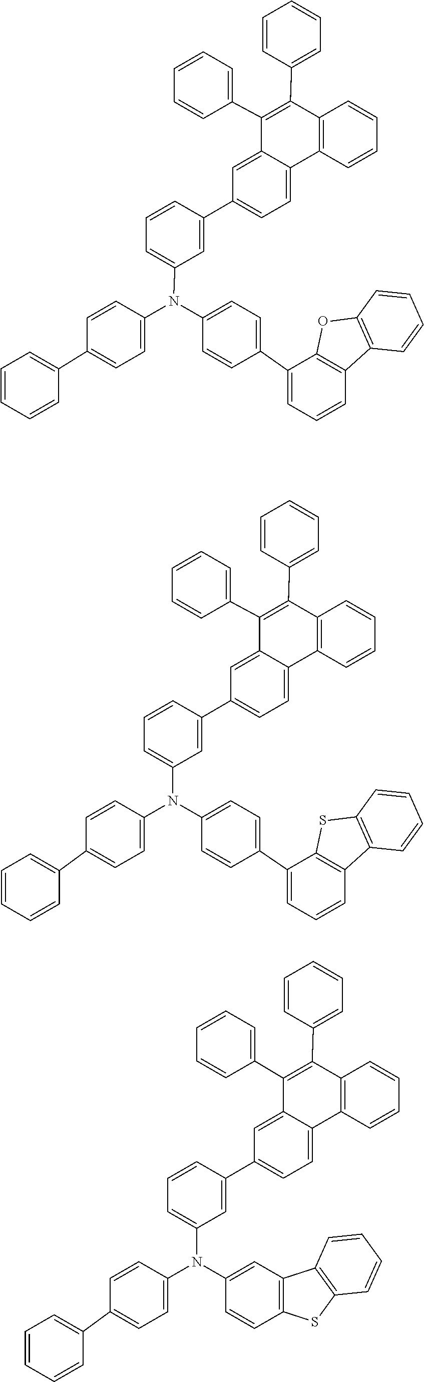

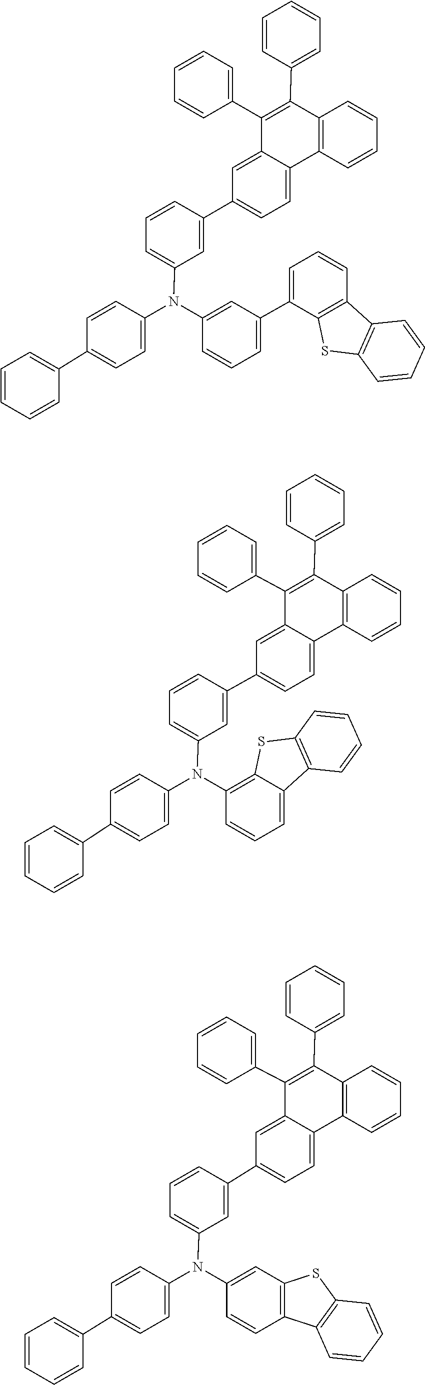 [SMILES: c1ccc(-c2ccc(N(c3cccc(-c4ccc5c(c4)c(-c4ccccc4)c(-c4ccccc4)c4ccccc45)c3)c3cccc(-c4cccc5c4sc4ccccc45)c3)cc2)cc1 ; c1ccc(-c2ccc(N(c3cccc(-c4ccc5c(c4)c(-c4ccccc4)c(-c4ccccc4)c4ccccc45)c3)c3cccc4c3sc3ccccc34)cc2)cc1 ; c1ccc(-c2ccc(N(c3cccc(-c4ccc5c(c4)c(-c4ccccc4)c(-c4ccccc4)c4ccccc45)c3)c3ccc4c(c3)sc3ccccc34)cc2)cc1]

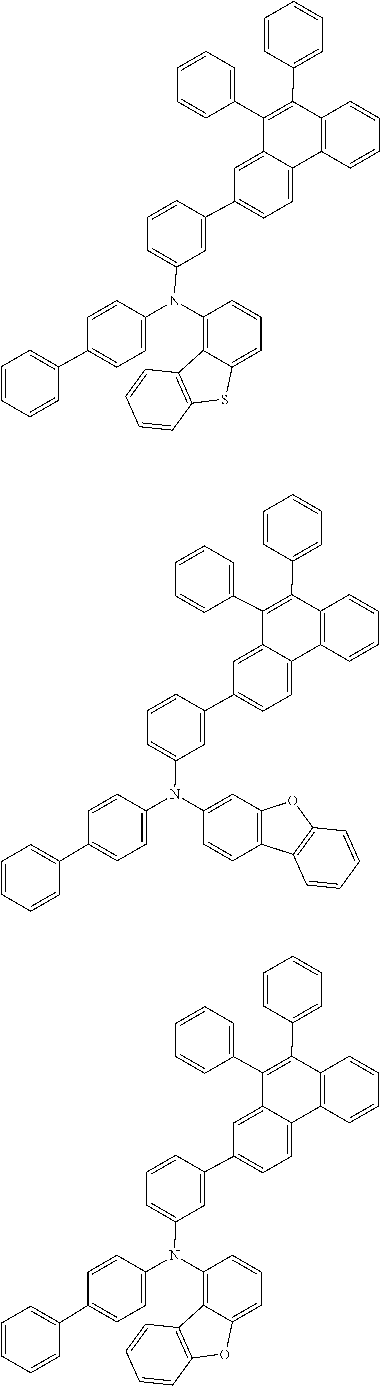 [SMILES: c1ccc(-c2ccc(N(c3cccc(-c4ccc5c(c4)c(-c4ccccc4)c(-c4ccccc4)c4ccccc45)c3)c3cccc4sc5ccccc5c34)cc2)cc1 ; c1ccc(-c2ccc(N(c3cccc(-c4ccc5c(c4)c(-c4ccccc4)c(-c4ccccc4)c4ccccc45)c3)c3ccc4c(c3)oc3ccccc34)cc2)cc1 ; c1ccc(-c2ccc(N(c3cccc(-c4ccc5c(c4)c(-c4ccccc4)c(-c4ccccc4)c4ccccc45)c3)c3cccc4oc5ccccc5c34)cc2)cc1]

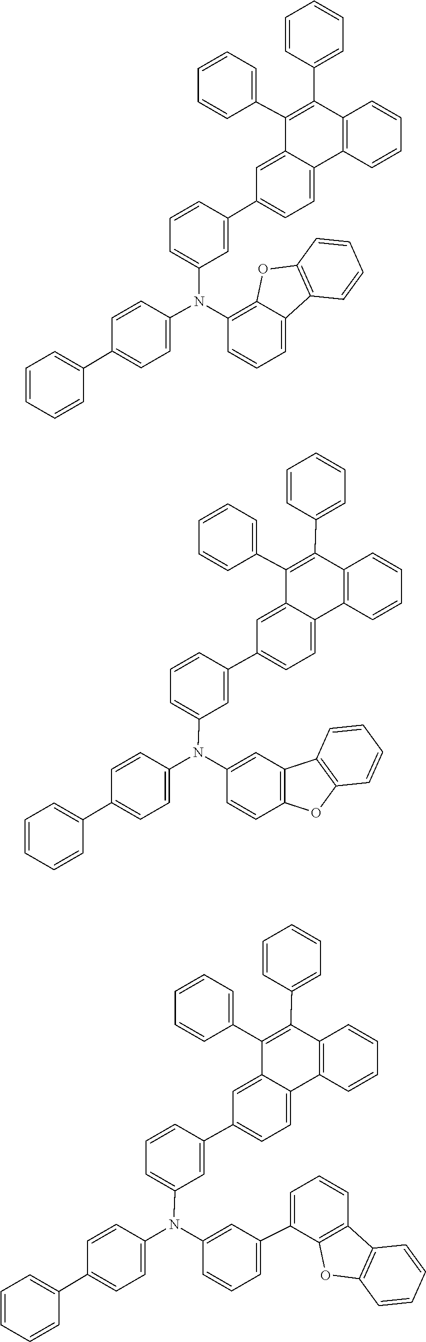 [SMILES: c1ccc(-c2ccc(N(c3cccc(-c4ccc5c(c4)c(-c4ccccc4)c(-c4ccccc4)c4ccccc45)c3)c3cccc4c3oc3ccccc34)cc2)cc1 ; c1ccc(-c2ccc(N(c3cccc(-c4ccc5c(c4)c(-c4ccccc4)c(-c4ccccc4)c4ccccc45)c3)c3ccc4oc5ccccc5c4c3)cc2)cc1 ; c1ccc(-c2ccc(N(c3cccc(-c4ccc5c(c4)c(-c4ccccc4)c(-c4ccccc4)c4ccccc45)c3)c3cccc(-c4cccc5c4oc4ccccc45)c3)cc2)cc1]

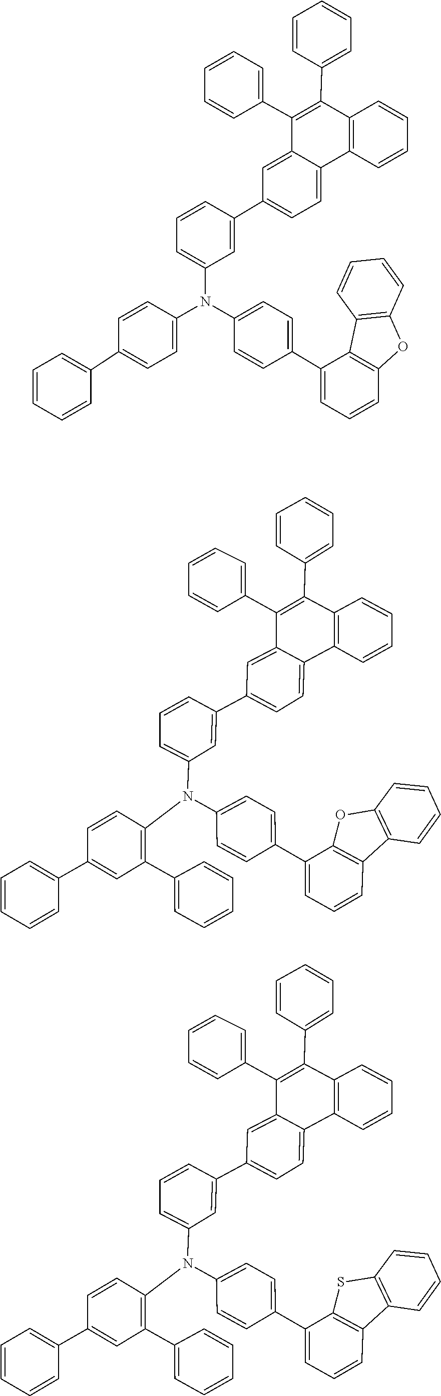 [SMILES: c1ccc(-c2ccc(N(c3ccc(-c4cccc5oc6ccccc6c45)cc3)c3cccc(-c4ccc5c(c4)c(-c4ccccc4)c(-c4ccccc4)c4ccccc45)c3)cc2)cc1 ; c1ccc(-c2ccc(N(c3ccc(-c4cccc5c4oc4ccccc45)cc3)c3cccc(-c4ccc5c(c4)c(-c4ccccc4)c(-c4ccccc4)c4ccccc45)c3)c(-c3ccccc3)c2)cc1 ; c1ccc(-c2ccc(N(c3ccc(-c4cccc5c4sc4ccccc45)cc3)c3cccc(-c4ccc5c(c4)c(-c4ccccc4)c(-c4ccccc4)c4ccccc45)c3)c(-c3ccccc3)c2)cc1]

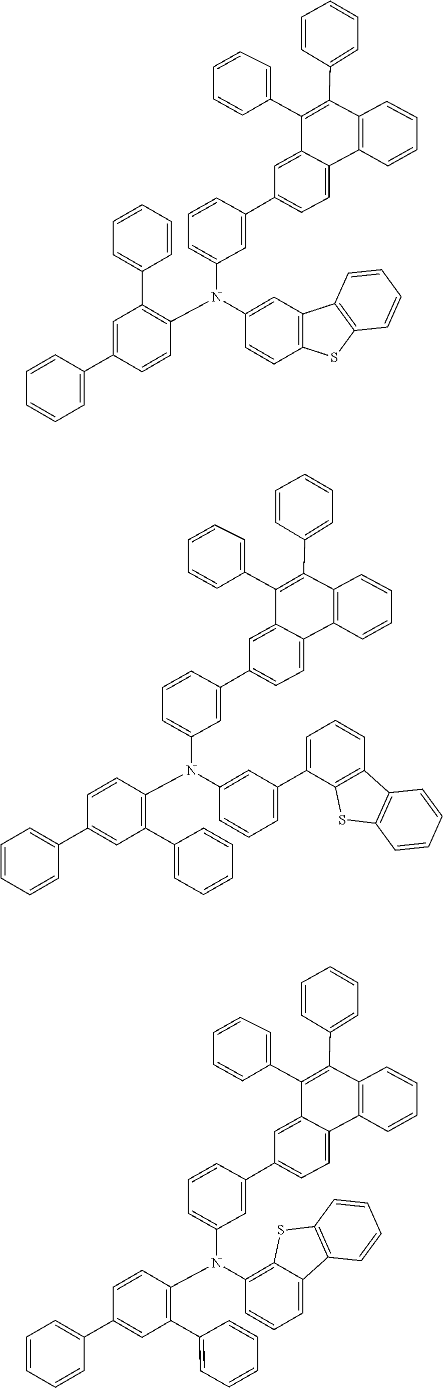 [SMILES: c1ccc(-c2ccc(N(c3cccc(-c4ccc5c(c4)c(-c4ccccc4)c(-c4ccccc4)c4ccccc45)c3)c3ccc4sc5ccccc5c4c3)c(-c3ccccc3)c2)cc1 ; c1ccc(-c2ccc(N(c3cccc(-c4ccc5c(c4)c(-c4ccccc4)c(-c4ccccc4)c4ccccc45)c3)c3cccc(-c4cccc5c4sc4ccccc45)c3)c(-c3ccccc3)c2)cc1 ; c1ccc(-c2ccc(N(c3cccc(-c4ccc5c(c4)c(-c4ccccc4)c(-c4ccccc4)c4ccccc45)c3)c3cccc4c3sc3ccccc34)c(-c3ccccc3)c2)cc1]

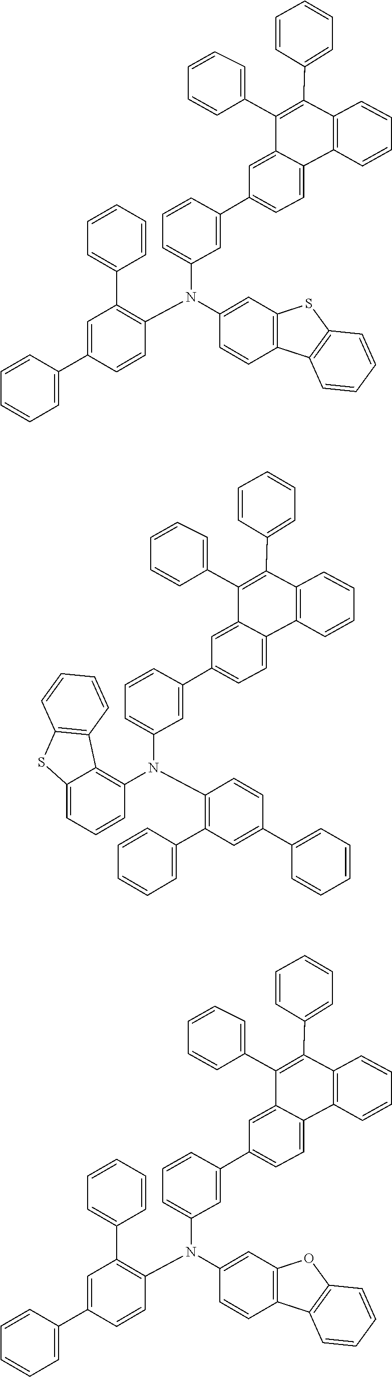 [SMILES: c1ccc(-c2ccc(N(c3cccc(-c4ccc5c(c4)c(-c4ccccc4)c(-c4ccccc4)c4ccccc45)c3)c3ccc4c(c3)sc3ccccc34)c(-c3ccccc3)c2)cc1 ; c1ccc(-c2ccc(N(c3cccc(-c4ccc5c(c4)c(-c4ccccc4)c(-c4ccccc4)c4ccccc45)c3)c3cccc4sc5ccccc5c34)c(-c3ccccc3)c2)cc1 ; c1ccc(-c2ccc(N(c3cccc(-c4ccc5c(c4)c(-c4ccccc4)c(-c4ccccc4)c4ccccc45)c3)c3ccc4c(c3)oc3ccccc34)c(-c3ccccc3)c2)cc1]

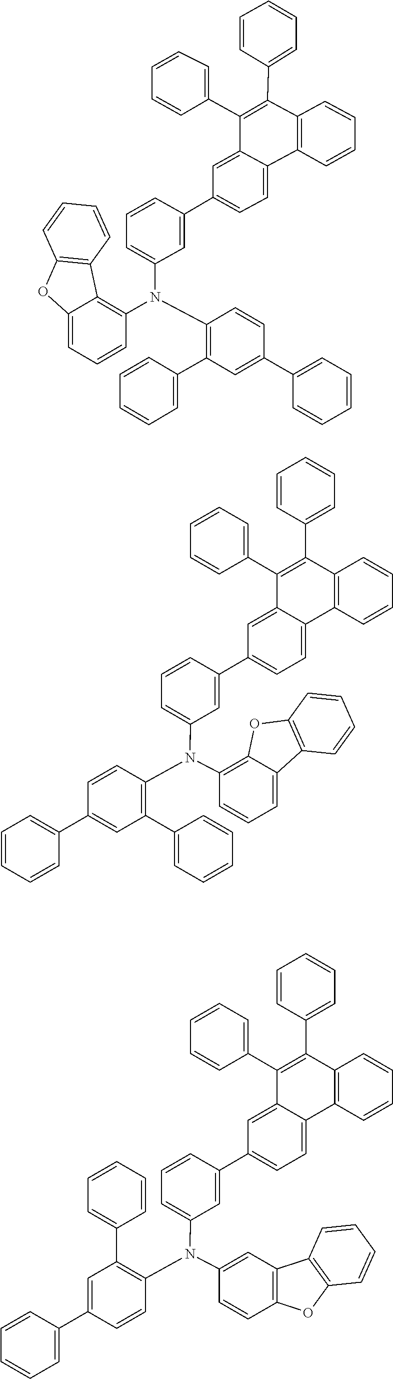

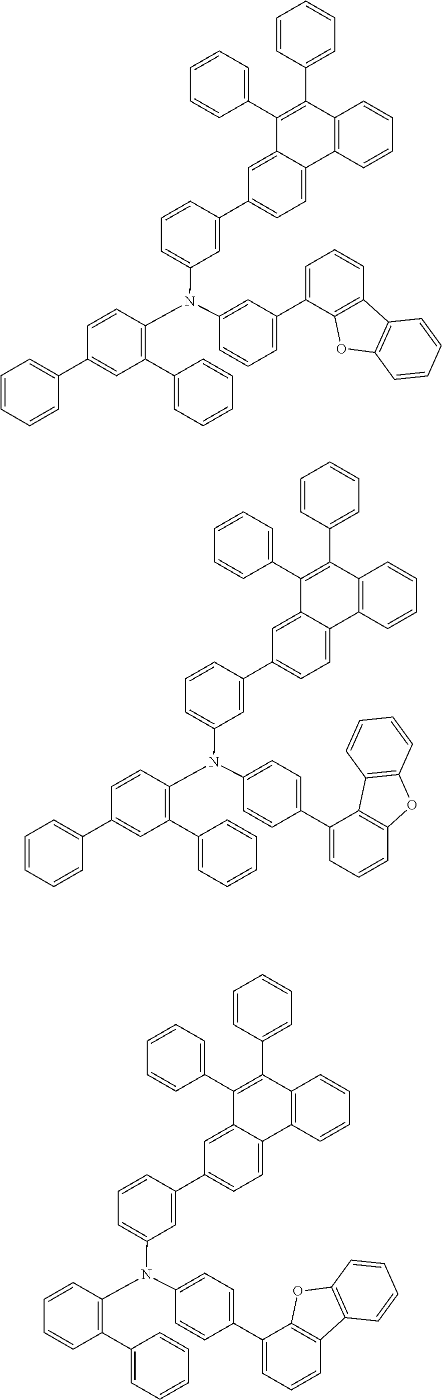 [SMILES: c1ccc(-c2ccc(N(c3cccc(-c4ccc5c(c4)c(-c4ccccc4)c(-c4ccccc4)c4ccccc45)c3)c3cccc(-c4cccc5c4oc4ccccc45)c3)c(-c3ccccc3)c2)cc1 ; c1ccc(-c2ccc(N(c3ccc(-c4cccc5oc6ccccc6c45)cc3)c3cccc(-c4ccc5c(c4)c(-c4ccccc4)c(-c4ccccc4)c4ccccc45)c3)c(-c3ccccc3)c2)cc1 ; c1ccc(-c2ccccc2N(c2ccc(-c3cccc4c3oc3ccccc34)cc2)c2cccc(-c3ccc4c(c3)c(-c3ccccc3)c(-c3ccccc3)c3ccccc34)c2)cc1]

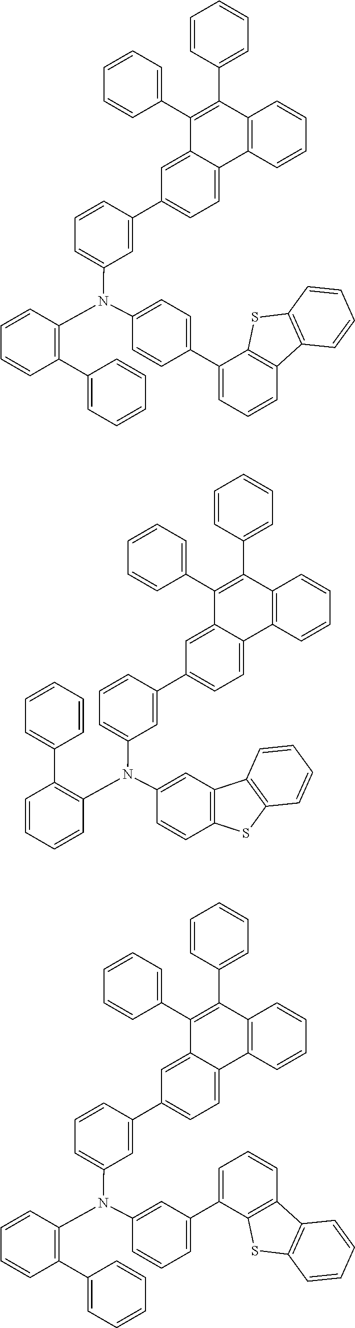 [SMILES: c1ccc(-c2ccccc2N(c2ccc(-c3cccc4c3sc3ccccc34)cc2)c2cccc(-c3ccc4c(c3)c(-c3ccccc3)c(-c3ccccc3)c3ccccc34)c2)cc1 ; c1ccc(-c2ccccc2N(c2cccc(-c3ccc4c(c3)c(-c3ccccc3)c(-c3ccccc3)c3ccccc34)c2)c2ccc3sc4ccccc4c3c2)cc1 ; c1ccc(-c2ccccc2N(c2cccc(-c3ccc4c(c3)c(-c3ccccc3)c(-c3ccccc3)c3ccccc34)c2)c2cccc(-c3cccc4c3sc3ccccc34)c2)cc1]

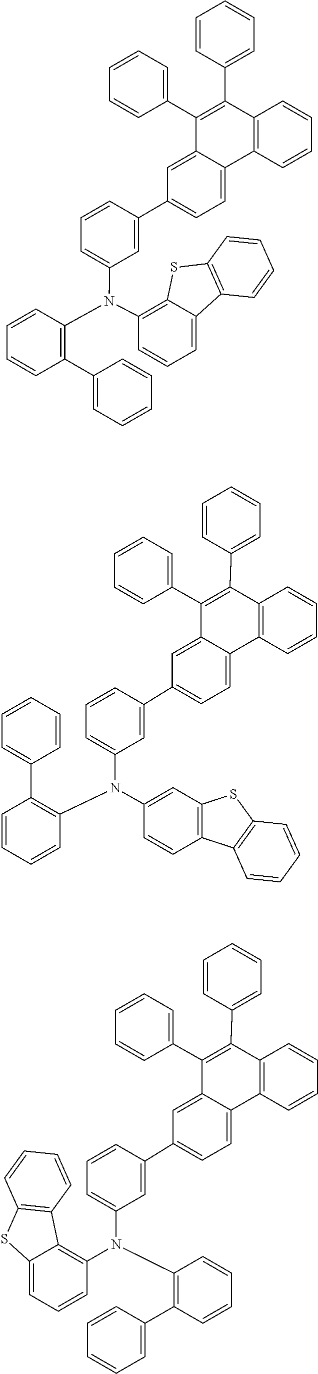 [SMILES: c1ccc(-c2ccccc2N(c2cccc(-c3ccc4c(c3)c(-c3ccccc3)c(-c3ccccc3)c3ccccc34)c2)c2cccc3c2sc2ccccc23)cc1 ; c1ccc(-c2ccccc2N(c2cccc(-c3ccc4c(c3)c(-c3ccccc3)c(-c3ccccc3)c3ccccc34)c2)c2ccc3c(c2)sc2ccccc23)cc1 ; c1ccc(-c2ccccc2N(c2cccc(-c3ccc4c(c3)c(-c3ccccc3)c(-c3ccccc3)c3ccccc34)c2)c2cccc3sc4ccccc4c23)cc1]

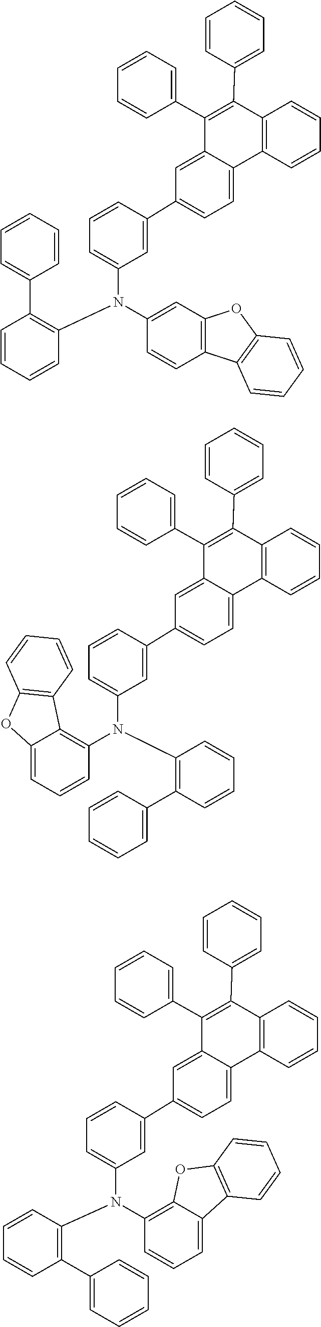

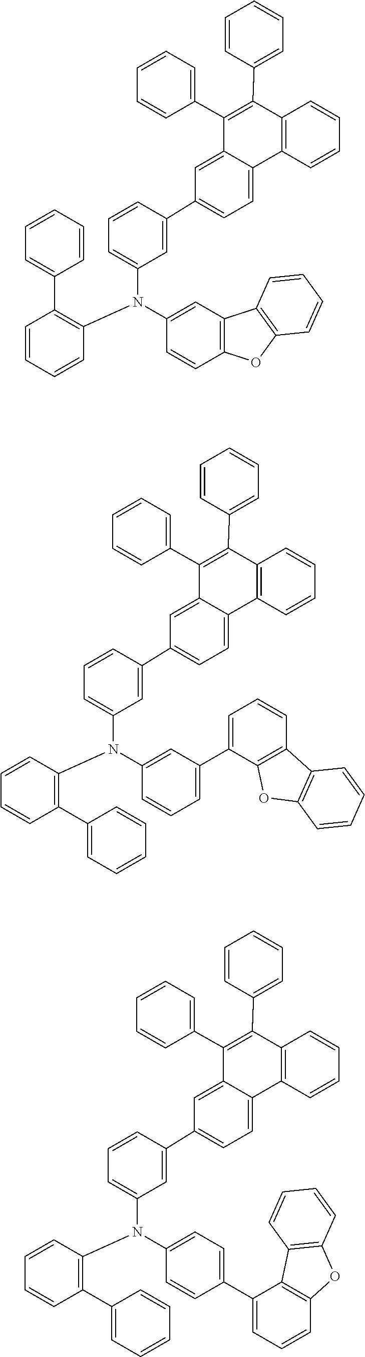 [SMILES: c1ccc(-c2ccccc2N(c2cccc(-c3ccc4c(c3)c(-c3ccccc3)c(-c3ccccc3)c3ccccc34)c2)c2ccc3oc4ccccc4c3c2)cc1 ; c1ccc(-c2ccccc2N(c2cccc(-c3ccc4c(c3)c(-c3ccccc3)c(-c3ccccc3)c3ccccc34)c2)c2cccc(-c3cccc4c3oc3ccccc34)c2)cc1 ; c1ccc(-c2ccccc2N(c2ccc(-c3cccc4oc5ccccc5c34)cc2)c2cccc(-c3ccc4c(c3)c(-c3ccccc3)c(-c3ccccc3)c3ccccc34)c2)cc1]

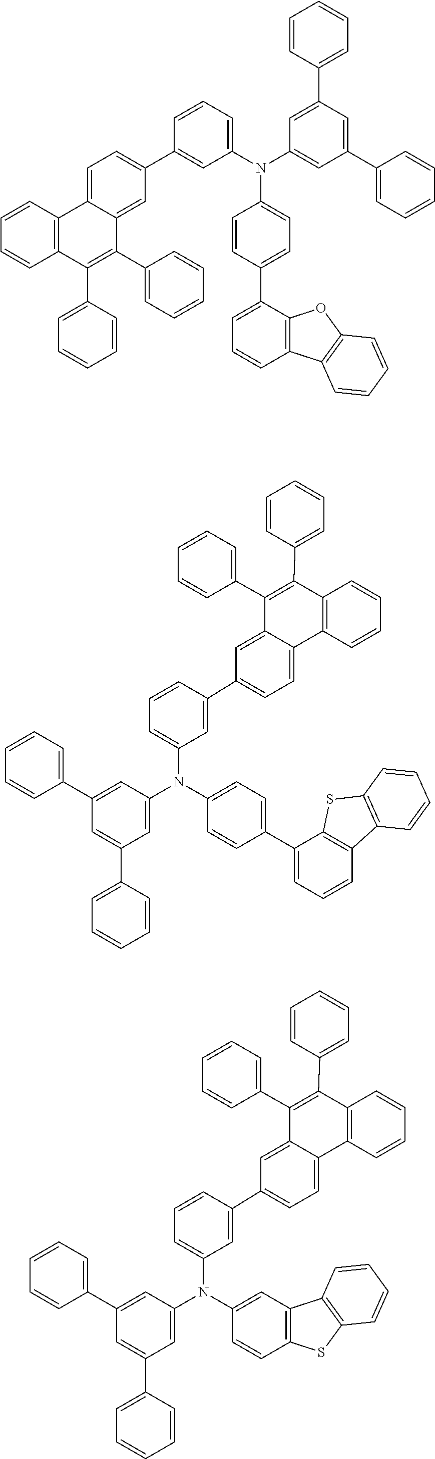 [SMILES: c1ccc(-c2cc(-c3ccccc3)cc(N(c3ccc(-c4cccc5c4oc4ccccc45)cc3)c3cccc(-c4ccc5c(c4)c(-c4ccccc4)c(-c4ccccc4)c4ccccc45)c3)c2)cc1 ; c1ccc(-c2cc(-c3ccccc3)cc(N(c3ccc(-c4cccc5c4sc4ccccc45)cc3)c3cccc(-c4ccc5c(c4)c(-c4ccccc4)c(-c4ccccc4)c4ccccc45)c3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3ccc4sc5ccccc5c4c3)c2)cc1]

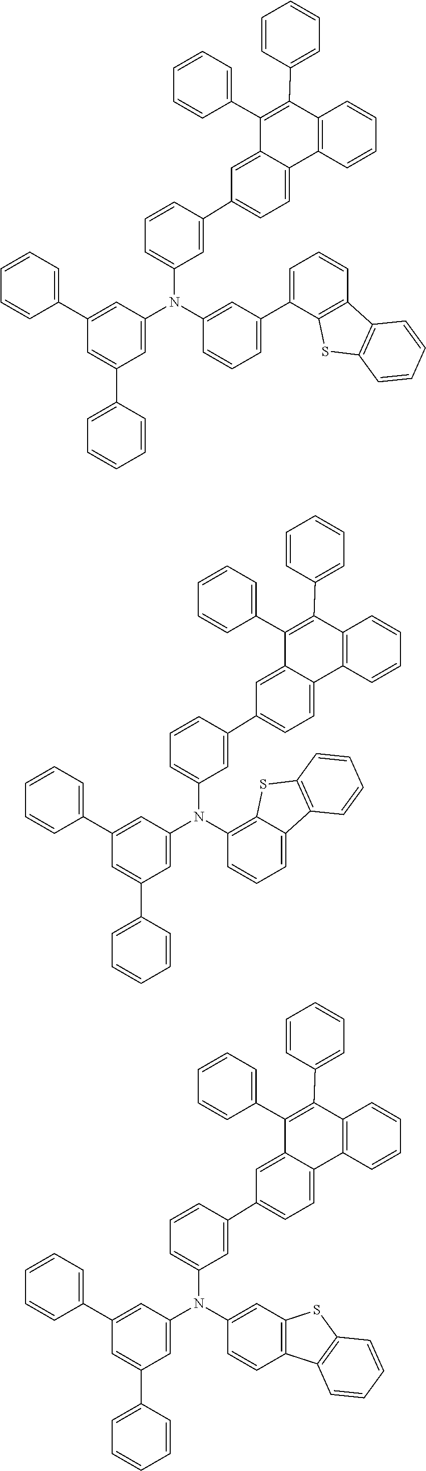 [SMILES: c1ccc(-c2cc(-c3ccccc3)cc(N(c3cccc(-c4ccc5c(c4)c(-c4ccccc4)c(-c4ccccc4)c4ccccc45)c3)c3cccc(-c4cccc5c4sc4ccccc45)c3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3cccc4c3sc3ccccc34)c2)cc1 ; c1ccc(-c2cc(-c3ccccc3)cc(N(c3cccc(-c4ccc5c(c4)c(-c4ccccc4)c(-c4ccccc4)c4ccccc45)c3)c3ccc4c(c3)sc3ccccc34)c2)cc1]

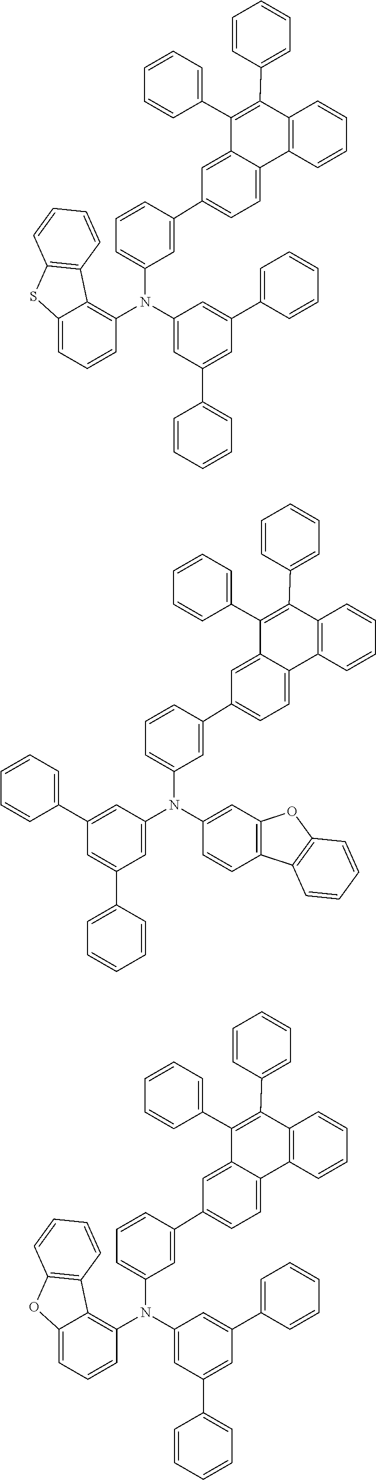 [SMILES: c1ccc(-c2cc(-c3ccccc3)cc(N(c3cccc(-c4ccc5c(c4)c(-c4ccccc4)c(-c4ccccc4)c4ccccc45)c3)c3cccc4sc5ccccc5c34)c2)cc1 ; c1ccc(-c2cc(-c3ccccc3)cc(N(c3cccc(-c4ccc5c(c4)c(-c4ccccc4)c(-c4ccccc4)c4ccccc45)c3)c3ccc4c(c3)oc3ccccc34)c2)cc1 ; c1ccc(-c2cc(-c3ccccc3)cc(N(c3cccc(-c4ccc5c(c4)c(-c4ccccc4)c(-c4ccccc4)c4ccccc45)c3)c3cccc4oc5ccccc5c34)c2)cc1]

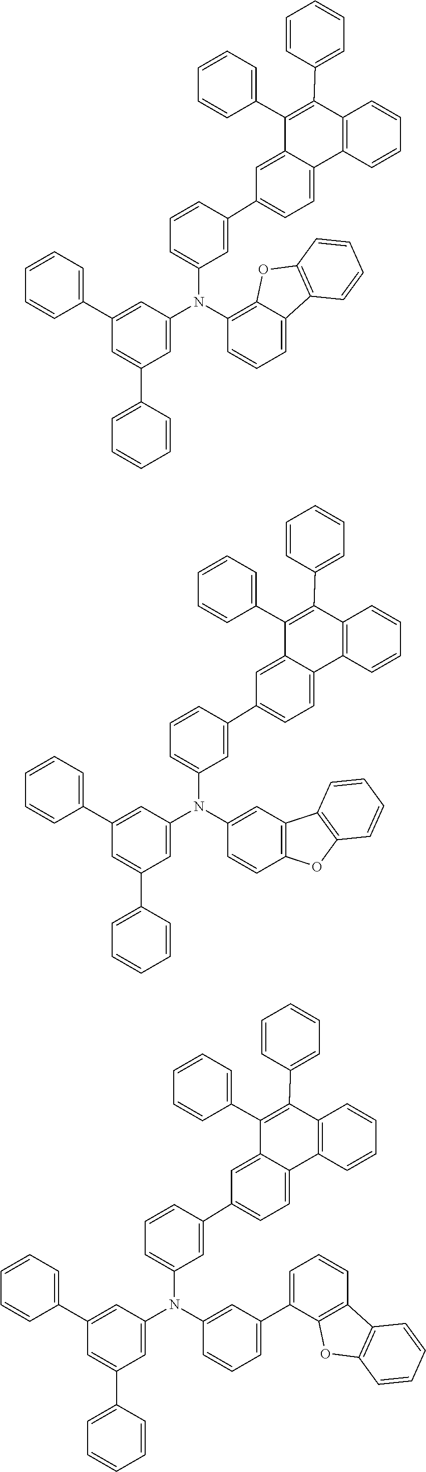 [SMILES: c1ccc(-c2cc(-c3ccccc3)cc(N(c3cccc(-c4ccc5c(c4)c(-c4ccccc4)c(-c4ccccc4)c4ccccc45)c3)c3cccc4c3oc3ccccc34)c2)cc1 ; c1ccc(-c2cc(-c3ccccc3)cc(N(c3cccc(-c4ccc5c(c4)c(-c4ccccc4)c(-c4ccccc4)c4ccccc45)c3)c3ccc4oc5ccccc5c4c3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3cccc(-c4cccc5c4oc4ccccc45)c3)c2)cc1]

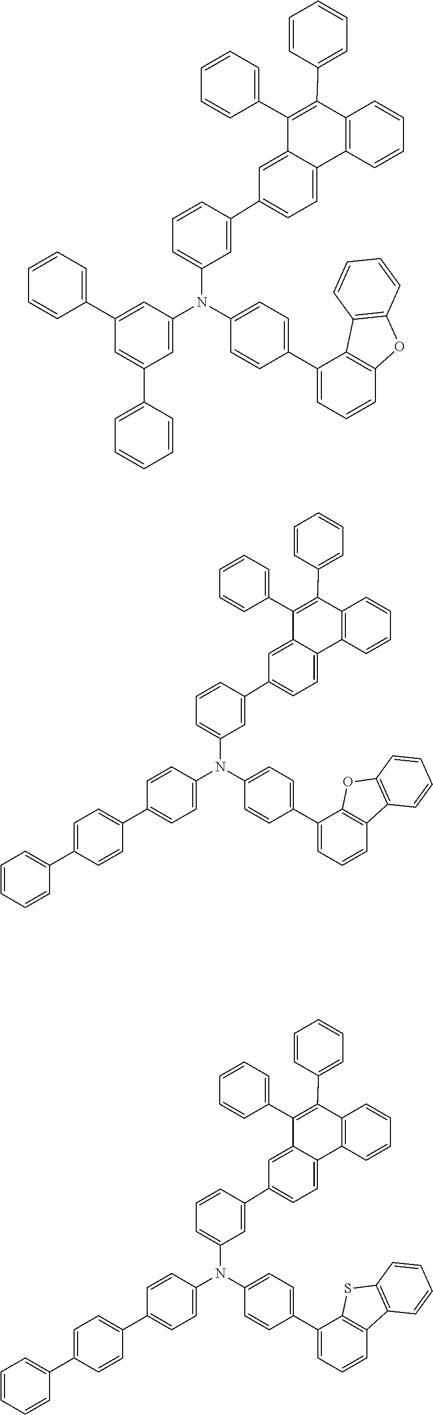 [SMILES: c1ccc(-c2cc(-c3ccccc3)cc(N(c3ccc(-c4cccc5oc6ccccc6c45)cc3)c3cccc(-c4ccc5c(c4)c(-c4ccccc4)c(-c4ccccc4)c4ccccc45)c3)c2)cc1 ; c1ccc(-c2ccc(-c3ccc(N(c4ccc(-c5cccc6c5oc5ccccc56)cc4)c4cccc(-c5ccc6c(c5)c(-c5ccccc5)c(-c5ccccc5)c5ccccc56)c4)cc3)cc2)cc1 ; c1ccc(-c2ccc(-c3ccc(N(c4ccc(-c5cccc6c5sc5ccccc56)cc4)c4cccc(-c5ccc6c(c5)c(-c5ccccc5)c(-c5ccccc5)c5ccccc56)c4)cc3)cc2)cc1]

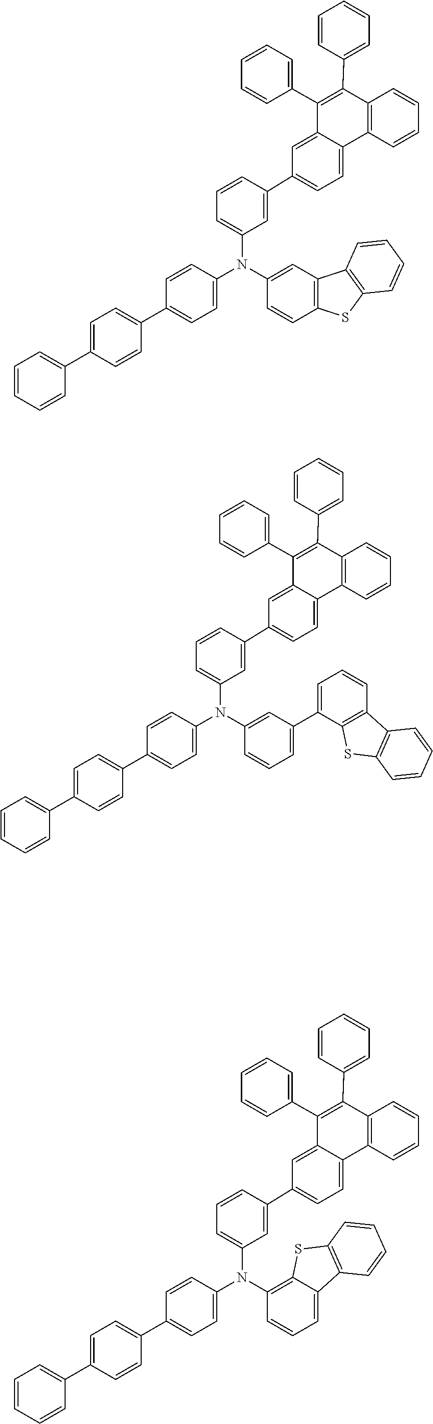 [SMILES: c1ccc(-c2ccc(-c3ccc(N(c4cccc(-c5ccc6c(c5)c(-c5ccccc5)c(-c5ccccc5)c5ccccc56)c4)c4ccc5sc6ccccc6c5c4)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4cccc(-c5cccc6c5sc5ccccc56)c4)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4cccc5c4sc4ccccc45)cc3)cc2)cc1]

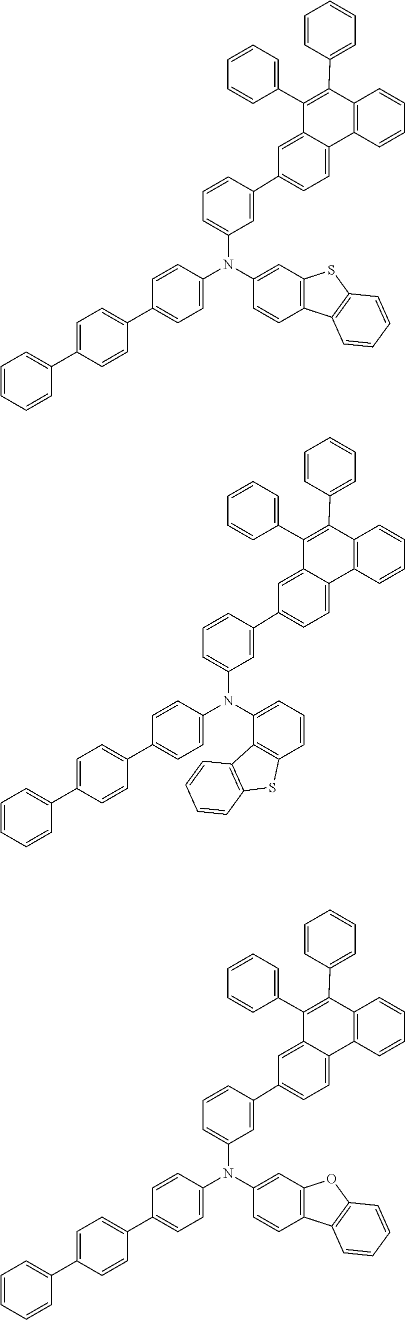 [SMILES: c1ccc(-c2ccc(-c3ccc(N(c4cccc(-c5ccc6c(c5)c(-c5ccccc5)c(-c5ccccc5)c5ccccc56)c4)c4ccc5c(c4)sc4ccccc45)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4cccc5sc6ccccc6c45)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4ccc5c(c4)oc4ccccc45)cc3)cc2)cc1]

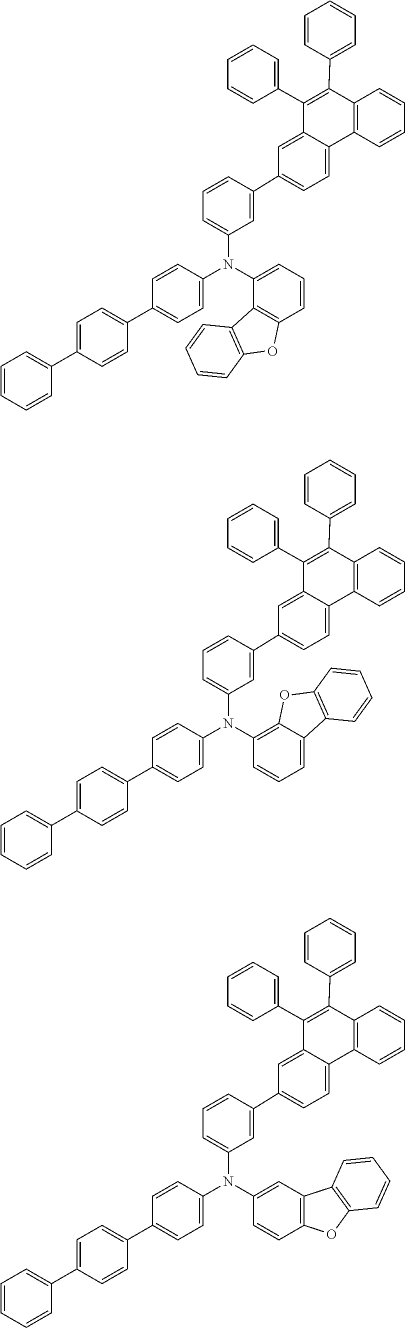 [SMILES: c1ccc(-c2ccc(-c3ccc(N(c4cccc(-c5ccc6c(c5)c(-c5ccccc5)c(-c5ccccc5)c5ccccc56)c4)c4cccc5oc6ccccc6c45)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4cccc5c4oc4ccccc45)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4ccc5oc6ccccc6c5c4)cc3)cc2)cc1]

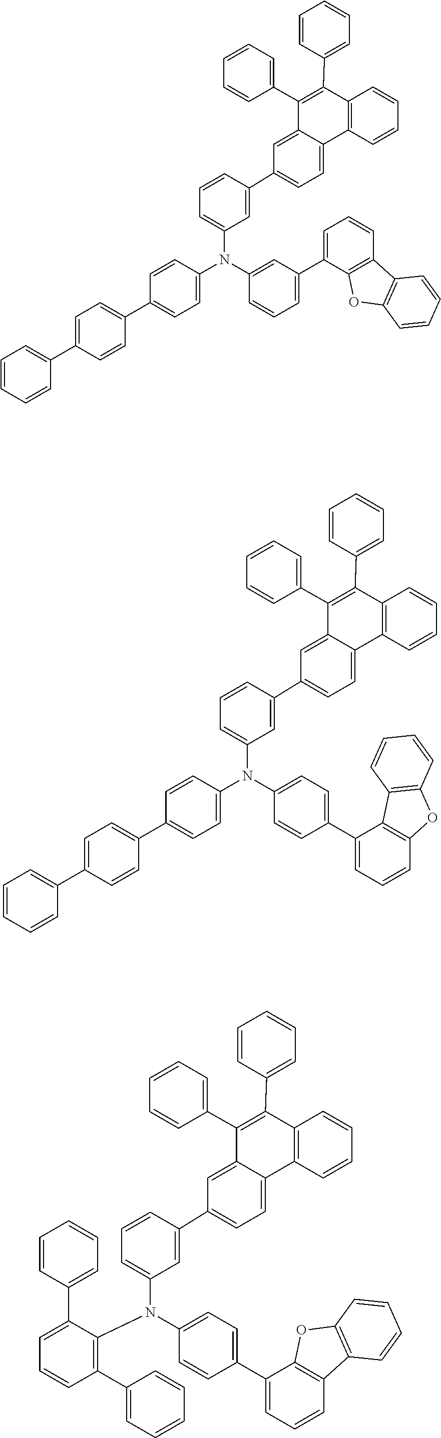 [SMILES: c1ccc(-c2ccc(-c3ccc(N(c4cccc(-c5ccc6c(c5)c(-c5ccccc5)c(-c5ccccc5)c5ccccc56)c4)c4cccc(-c5cccc6c5oc5ccccc56)c4)cc3)cc2)cc1 ; c1ccc(-c2ccc(-c3ccc(N(c4ccc(-c5cccc6oc7ccccc7c56)cc4)c4cccc(-c5ccc6c(c5)c(-c5ccccc5)c(-c5ccccc5)c5ccccc56)c4)cc3)cc2)cc1 ; c1ccc(-c2cccc(-c3ccccc3)c2N(c2ccc(-c3cccc4c3oc3ccccc34)cc2)c2cccc(-c3ccc4c(c3)c(-c3ccccc3)c(-c3ccccc3)c3ccccc34)c2)cc1]

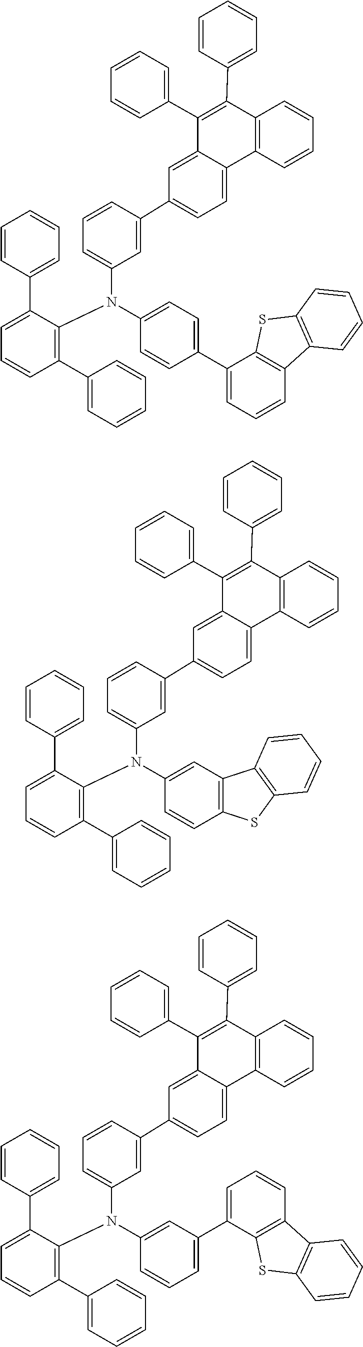

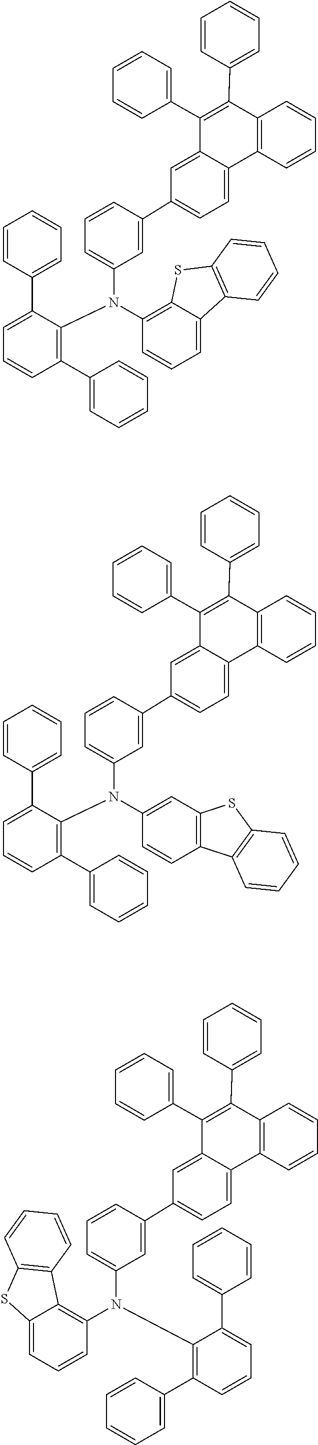 [SMILES: c1ccc(-c2cccc(-c3ccccc3)c2N(c2cccc(-c3ccc4c(c3)c(-c3ccccc3)c(-c3ccccc3)c3ccccc34)c2)c2cccc3c2sc2ccccc23)cc1 ; c1ccc(-c2cccc(-c3ccccc3)c2N(c2cccc(-c3ccc4c(c3)c(-c3ccccc3)c(-c3ccccc3)c3ccccc34)c2)c2ccc3c(c2)sc2ccccc23)cc1 ; c1ccc(-c2cccc(-c3ccccc3)c2N(c2cccc(-c3ccc4c(c3)c(-c3ccccc3)c(-c3ccccc3)c3ccccc34)c2)c2cccc3sc4ccccc4c23)cc1]

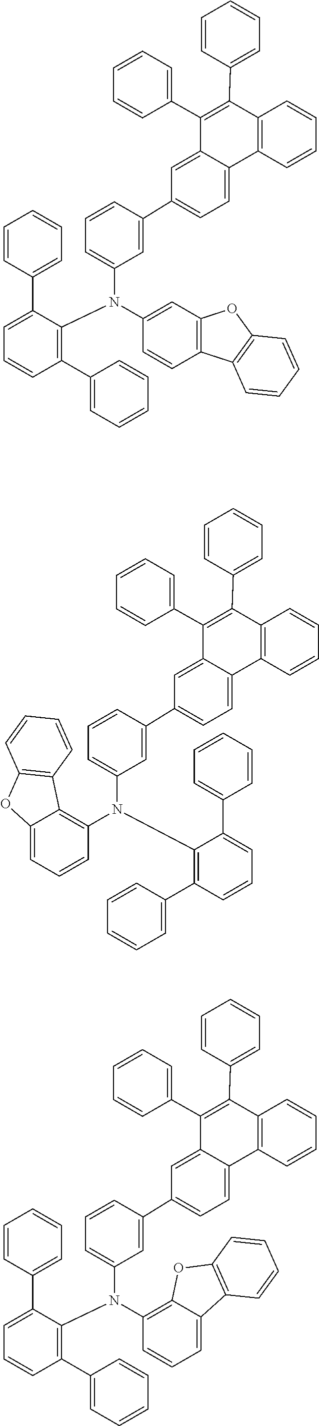 [SMILES: c1ccc(-c2cccc(-c3ccccc3)c2N(c2cccc(-c3ccc4c(c3)c(-c3ccccc3)c(-c3ccccc3)c3ccccc34)c2)c2ccc3c(c2)oc2ccccc23)cc1 ; c1ccc(-c2cccc(-c3ccccc3)c2N(c2cccc(-c3ccc4c(c3)c(-c3ccccc3)c(-c3ccccc3)c3ccccc34)c2)c2cccc3oc4ccccc4c23)cc1 ; c1ccc(-c2cccc(-c3ccccc3)c2N(c2cccc(-c3ccc4c(c3)c(-c3ccccc3)c(-c3ccccc3)c3ccccc34)c2)c2cccc3c2oc2ccccc23)cc1]

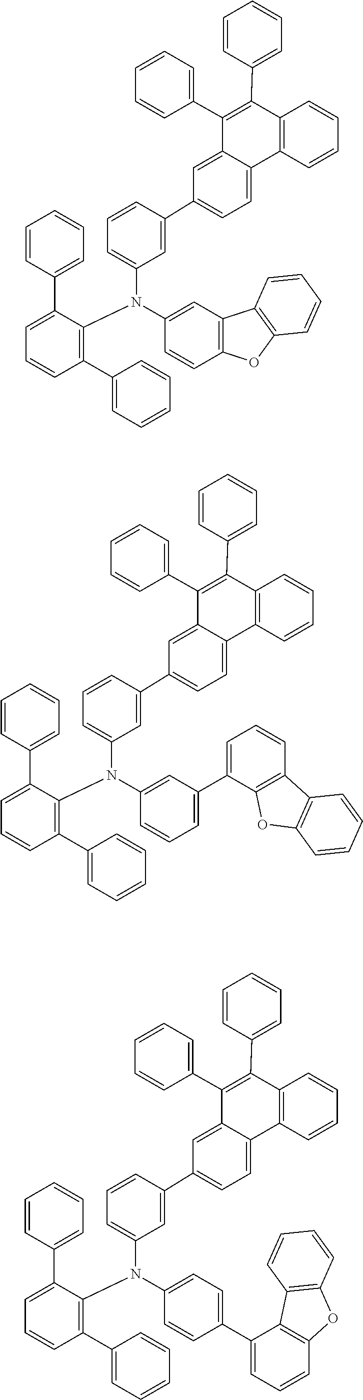 [SMILES: c1ccc(-c2cccc(-c3ccccc3)c2N(c2cccc(-c3ccc4c(c3)c(-c3ccccc3)c(-c3ccccc3)c3ccccc34)c2)c2ccc3oc4ccccc4c3c2)cc1 ; c1ccc(-c2cccc(-c3ccccc3)c2N(c2cccc(-c3ccc4c(c3)c(-c3ccccc3)c(-c3ccccc3)c3ccccc34)c2)c2cccc(-c3cccc4c3oc3ccccc34)c2)cc1 ; c1ccc(-c2cccc(-c3ccccc3)c2N(c2ccc(-c3cccc4oc5ccccc5c34)cc2)c2cccc(-c3ccc4c(c3)c(-c3ccccc3)c(-c3ccccc3)c3ccccc34)c2)cc1]

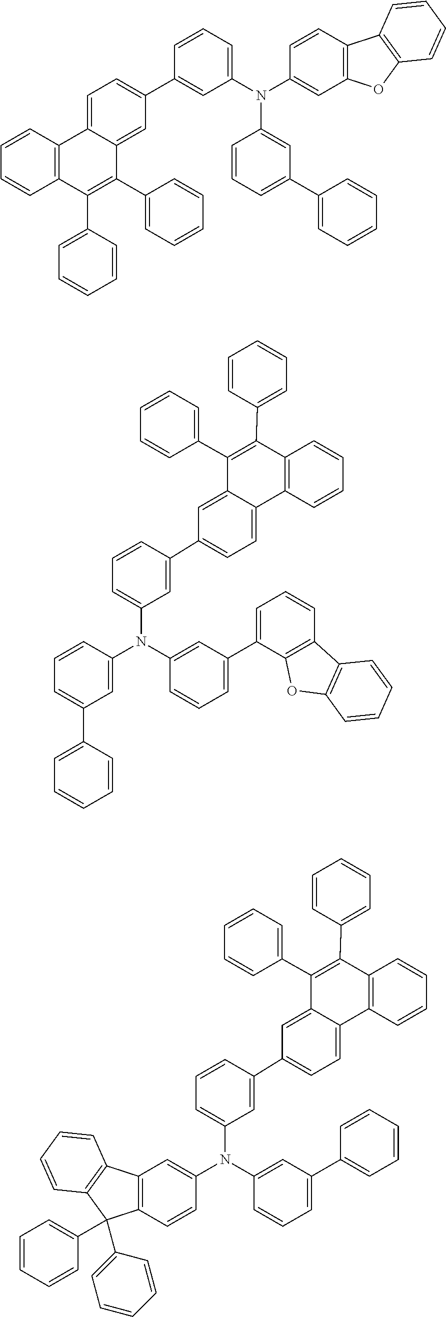 [SMILES: c1ccc(-c2cccc(N(c3cccc(-c4ccc5c(c4)c(-c4ccccc4)c(-c4ccccc4)c4ccccc45)c3)c3ccc4c(c3)oc3ccccc34)c2)cc1 ; c1ccc(-c2cccc(N(c3cccc(-c4ccc5c(c4)c(-c4ccccc4)c(-c4ccccc4)c4ccccc45)c3)c3cccc(-c4cccc5c4oc4ccccc45)c3)c2)cc1 ; c1ccc(-c2cccc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)c2)cc1]

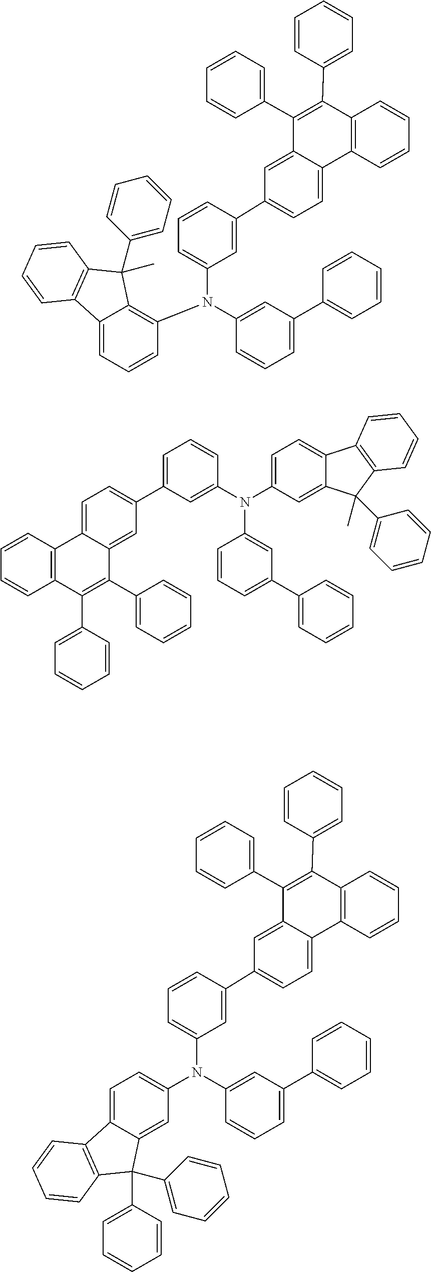 [SMILES: CC1(c2ccccc2)c2ccccc2-c2cccc(N(c3cccc(-c4ccccc4)c3)c3cccc(-c4ccc5c(c4)c(-c4ccccc4)c(-c4ccccc4)c4ccccc45)c3)c21 ; CC1(c2ccccc2)c2ccccc2-c2ccc(N(c3cccc(-c4ccccc4)c3)c3cccc(-c4ccc5c(c4)c(-c4ccccc4)c(-c4ccccc4)c4ccccc45)c3)cc21 ; c1ccc(-c2cccc(N(c3cccc(-c4ccc5c(c4)c(-c4ccccc4)c(-c4ccccc4)c4ccccc45)c3)c3ccc4c(c3)C(c3ccccc3)(c3ccccc3)c3ccccc3-4)c2)cc1]

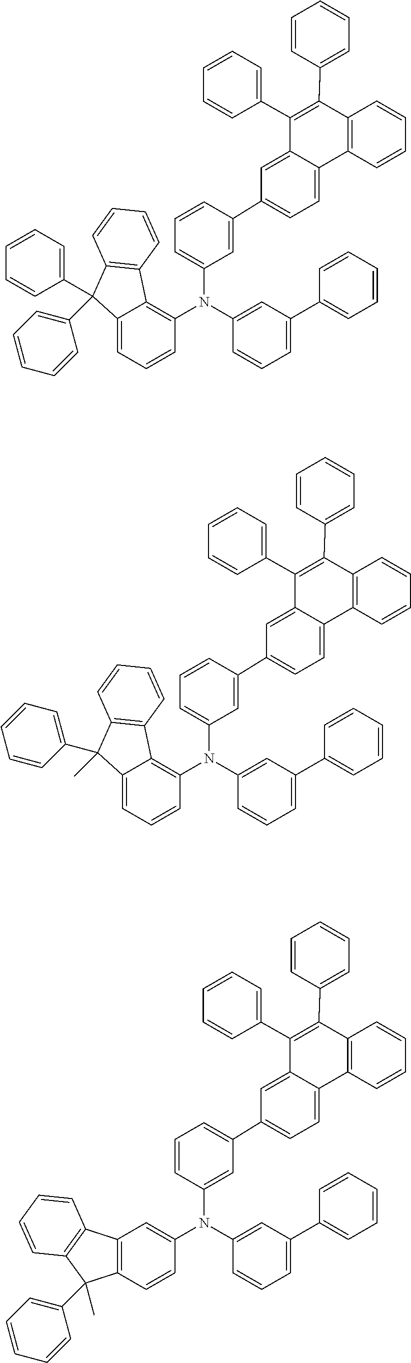 [SMILES: c1ccc(-c2cccc(N(c3cccc(-c4ccc5c(c4)c(-c4ccccc4)c(-c4ccccc4)c4ccccc45)c3)c3cccc4c3-c3ccccc3C4(c3ccccc3)c3ccccc3)c2)cc1 ; CC1(c2ccccc2)c2ccccc2-c2c(N(c3cccc(-c4ccccc4)c3)c3cccc(-c4ccc5c(c4)c(-c4ccccc4)c(-c4ccccc4)c4ccccc45)c3)cccc21 ; CC1(c2ccccc2)c2ccccc2-c2cc(N(c3cccc(-c4ccccc4)c3)c3cccc(-c4ccc5c(c4)c(-c4ccccc4)c(-c4ccccc4)c4ccccc45)c3)ccc21]

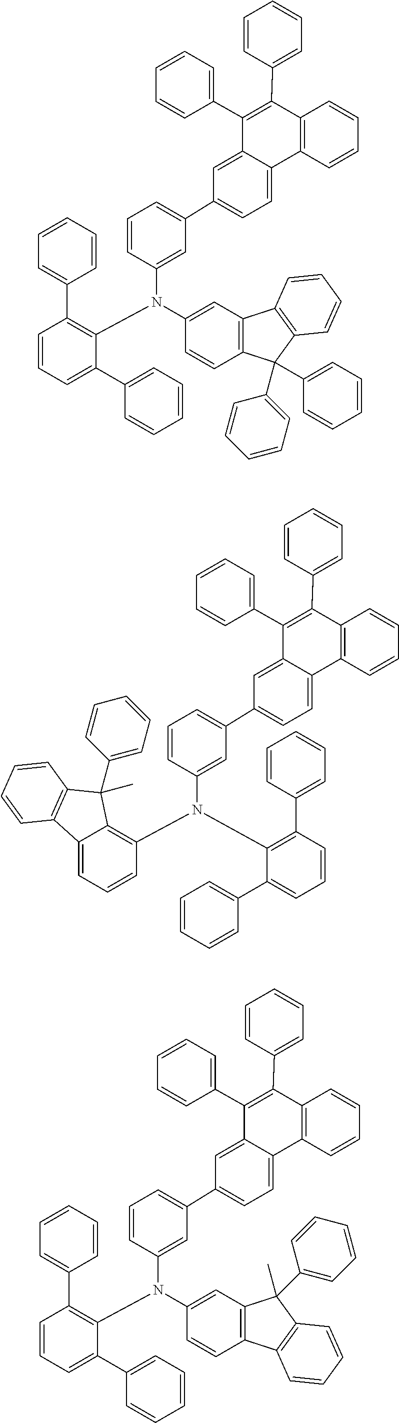 [SMILES: c1ccc(-c2cccc(-c3ccccc3)c2N(c2cccc(-c3ccc4c(c3)c(-c3ccccc3)c(-c3ccccc3)c3ccccc34)c2)c2ccc3c(c2)-c2ccccc2C3(c2ccccc2)c2ccccc2)cc1 ; CC1(c2ccccc2)c2ccccc2-c2cccc(N(c3cccc(-c4ccc5c(c4)c(-c4ccccc4)c(-c4ccccc4)c4ccccc45)c3)c3c(-c4ccccc4)cccc3-c3ccccc3)c21 ; CC1(c2ccccc2)c2ccccc2-c2ccc(N(c3cccc(-c4ccc5c(c4)c(-c4ccccc4)c(-c4ccccc4)c4ccccc45)c3)c3c(-c4ccccc4)cccc3-c3ccccc3)cc21]

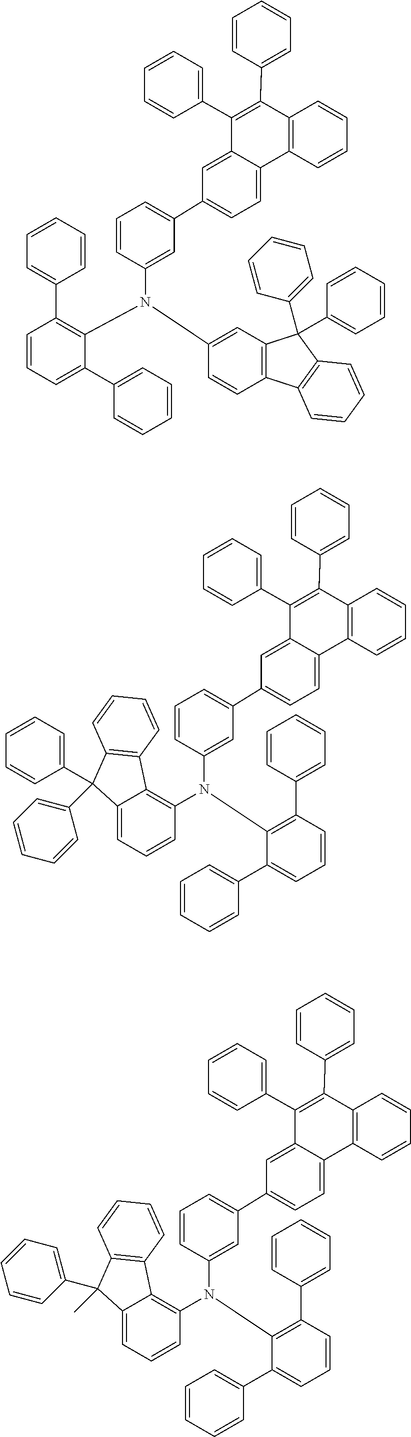 [SMILES: c1ccc(-c2cccc(-c3ccccc3)c2N(c2cccc(-c3ccc4c(c3)c(-c3ccccc3)c(-c3ccccc3)c3ccccc34)c2)c2ccc3c(c2)C(c2ccccc2)(c2ccccc2)c2ccccc2-3)cc1 ; c1ccc(-c2cccc(-c3ccccc3)c2N(c2cccc(-c3ccc4c(c3)c(-c3ccccc3)c(-c3ccccc3)c3ccccc34)c2)c2cccc3c2-c2ccccc2C3(c2ccccc2)c2ccccc2)cc1 ; CC1(c2ccccc2)c2ccccc2-c2c(N(c3cccc(-c4ccc5c(c4)c(-c4ccccc4)c(-c4ccccc4)c4ccccc45)c3)c3c(-c4ccccc4)cccc3-c3ccccc3)cccc21]

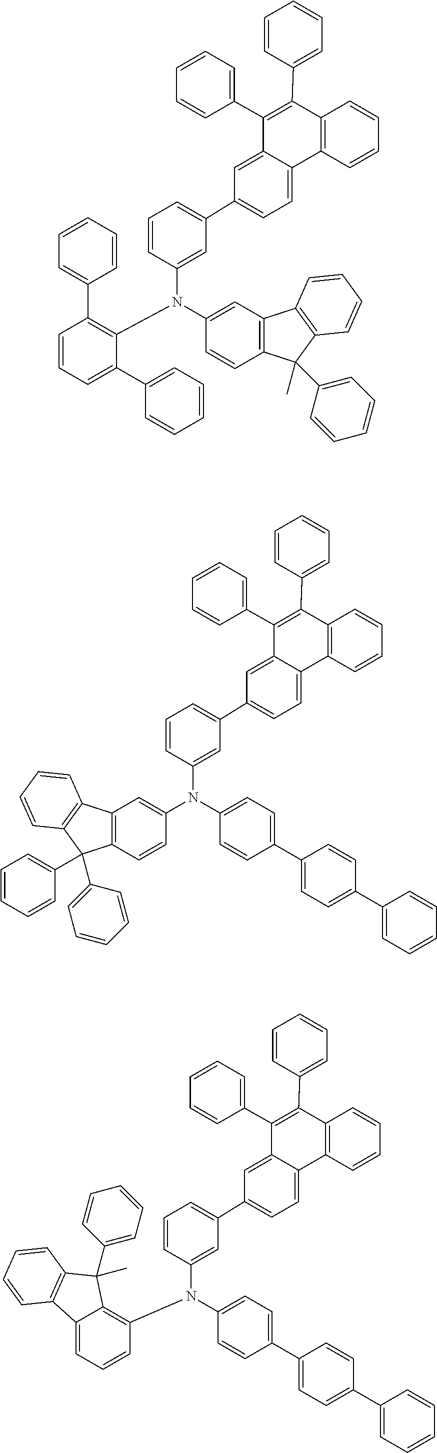 [SMILES: CC1(c2ccccc2)c2ccccc2-c2cc(N(c3cccc(-c4ccc5c(c4)c(-c4ccccc4)c(-c4ccccc4)c4ccccc45)c3)c3c(-c4ccccc4)cccc3-c3ccccc3)ccc21 ; c1ccc(-c2ccc(-c3ccc(N(c4cccc(-c5ccc6c(c5)c(-c5ccccc5)c(-c5ccccc5)c5ccccc56)c4)c4ccc5c(c4)-c4ccccc4C5(c4ccccc4)c4ccccc4)cc3)cc2)cc1 ; CC1(c2ccccc2)c2ccccc2-c2cccc(N(c3ccc(-c4ccc(-c5ccccc5)cc4)cc3)c3cccc(-c4ccc5c(c4)c(-c4ccccc4)c(-c4ccccc4)c4ccccc45)c3)c21]

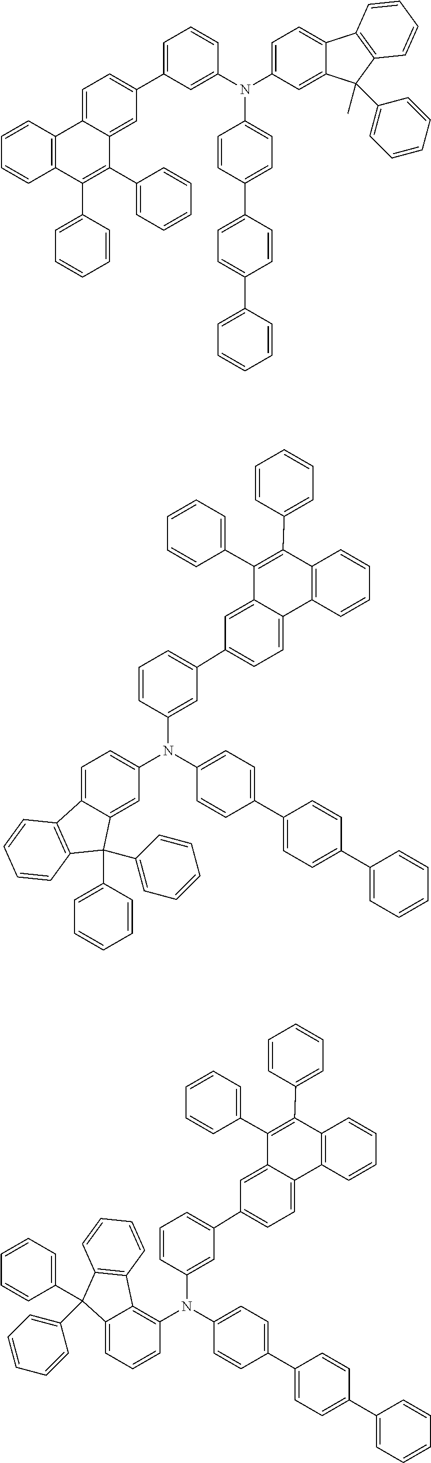 [SMILES: CC1(c2ccccc2)c2ccccc2-c2ccc(N(c3ccc(-c4ccc(-c5ccccc5)cc4)cc3)c3cccc(-c4ccc5c(c4)c(-c4ccccc4)c(-c4ccccc4)c4ccccc45)c3)cc21 ; c1ccc(-c2ccc(-c3ccc(N(c4cccc(-c5ccc6c(c5)c(-c5ccccc5)c(-c5ccccc5)c5ccccc56)c4)c4ccc5c(c4)C(c4ccccc4)(c4ccccc4)c4ccccc4-5)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4cccc5c4-c4ccccc4C5(c4ccccc4)c4ccccc4)cc3)cc2)cc1]

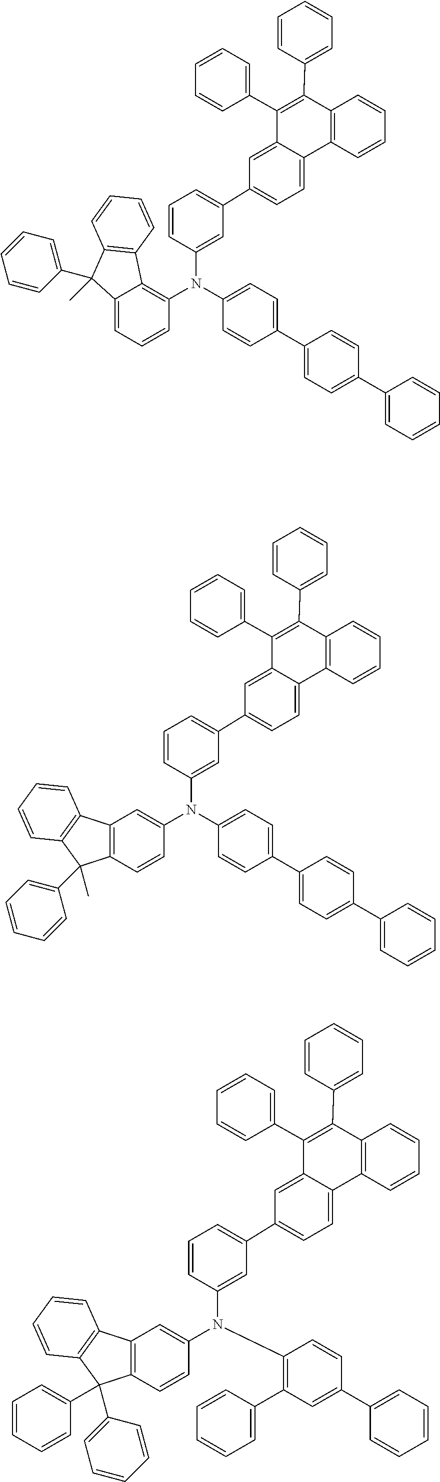 [SMILES: CC1(c2ccccc2)c2ccccc2-c2c(N(c3ccc(-c4ccc(-c5ccccc5)cc4)cc3)c3cccc(-c4ccc5c(c4)c(-c4ccccc4)c(-c4ccccc4)c4ccccc45)c3)cccc21 ; CC1(c2ccccc2)c2ccccc2-c2cc(N(c3ccc(-c4ccc(-c5ccccc5)cc4)cc3)c3cccc(-c4ccc5c(c4)c(-c4ccccc4)c(-c4ccccc4)c4ccccc45)c3)ccc21 ; c1ccc(-c2ccc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)c(-c3ccccc3)c2)cc1]

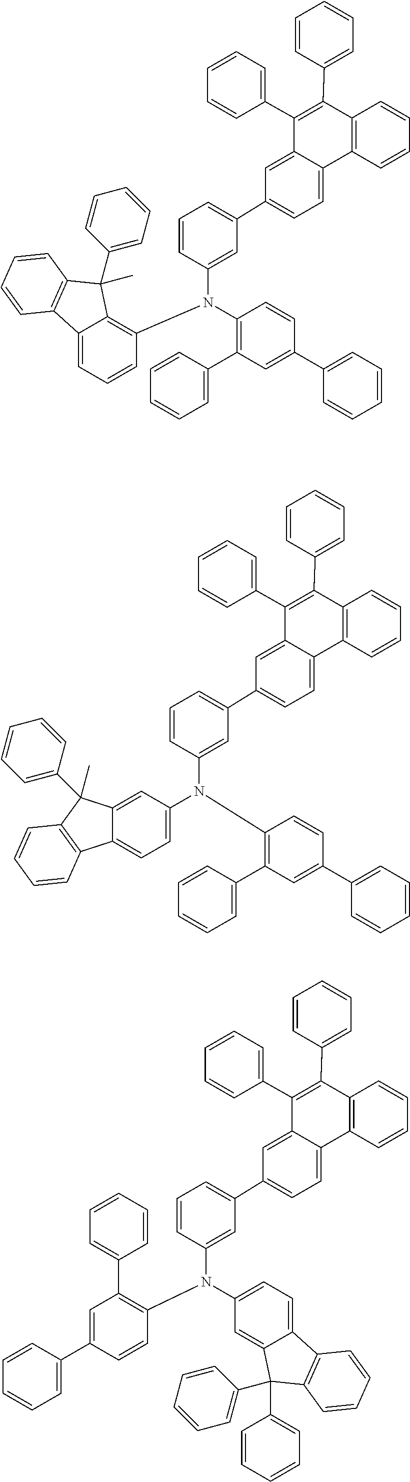 [SMILES: CC1(c2ccccc2)c2ccccc2-c2cccc(N(c3cccc(-c4ccc5c(c4)c(-c4ccccc4)c(-c4ccccc4)c4ccccc45)c3)c3ccc(-c4ccccc4)cc3-c3ccccc3)c21 ; CC1(c2ccccc2)c2ccccc2-c2ccc(N(c3cccc(-c4ccc5c(c4)c(-c4ccccc4)c(-c4ccccc4)c4ccccc45)c3)c3ccc(-c4ccccc4)cc3-c3ccccc3)cc21 ; c1ccc(-c2ccc(N(c3cccc(-c4ccc5c(c4)c(-c4ccccc4)c(-c4ccccc4)c4ccccc45)c3)c3ccc4c(c3)C(c3ccccc3)(c3ccccc3)c3ccccc3-4)c(-c3ccccc3)c2)cc1]

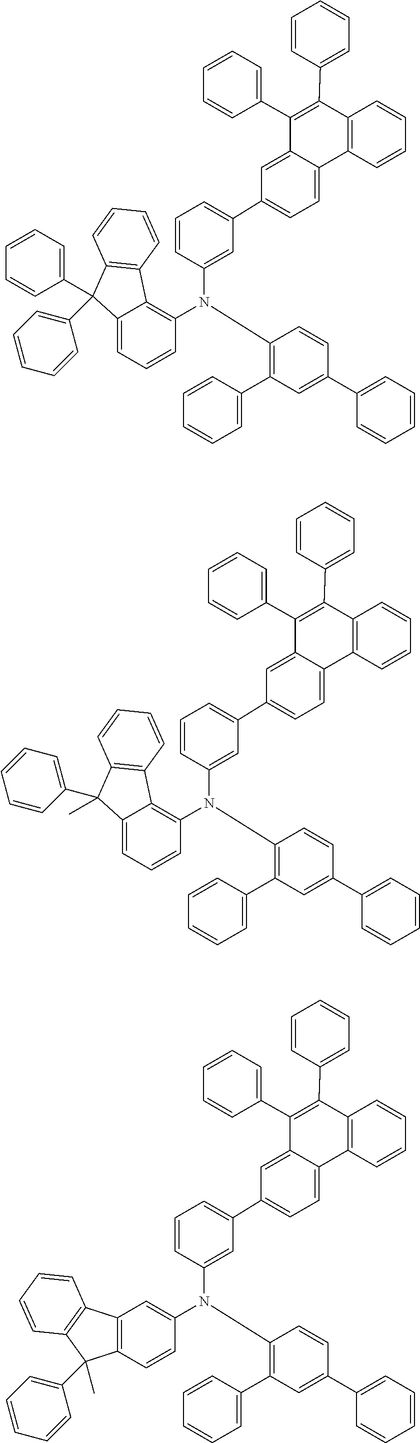 [SMILES: c1ccc(-c2ccc(N(c3cccc(-c4ccc5c(c4)c(-c4ccccc4)c(-c4ccccc4)c4ccccc45)c3)c3cccc4c3-c3ccccc3C4(c3ccccc3)c3ccccc3)c(-c3ccccc3)c2)cc1 ; CC1(c2ccccc2)c2ccccc2-c2c(N(c3cccc(-c4ccc5c(c4)c(-c4ccccc4)c(-c4ccccc4)c4ccccc45)c3)c3ccc(-c4ccccc4)cc3-c3ccccc3)cccc21 ; CC1(c2ccccc2)c2ccccc2-c2cc(N(c3cccc(-c4ccc5c(c4)c(-c4ccccc4)c(-c4ccccc4)c4ccccc45)c3)c3ccc(-c4ccccc4)cc3-c3ccccc3)ccc21]

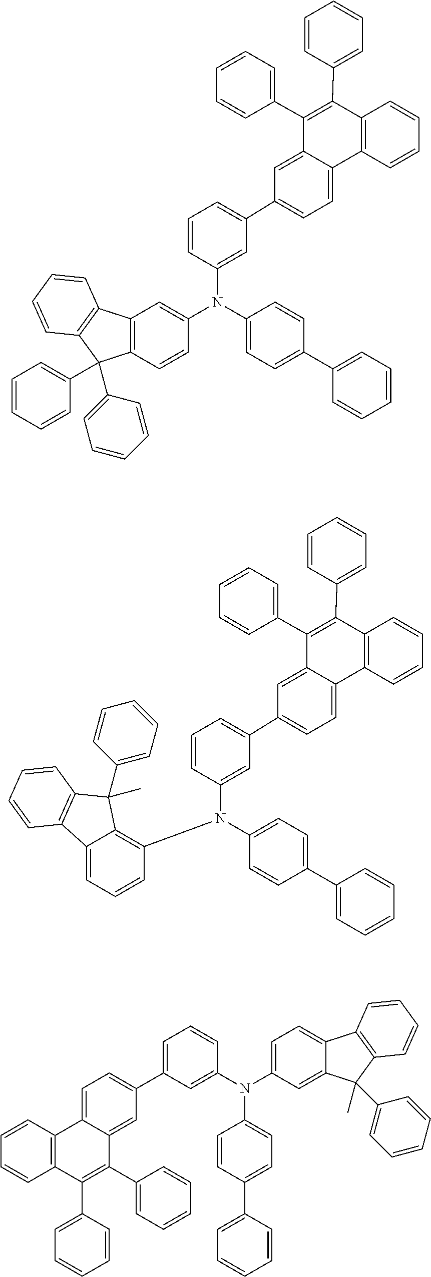 [SMILES: c1ccc(-c2ccc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)cc2)cc1 ; CC1(c2ccccc2)c2ccccc2-c2cccc(N(c3ccc(-c4ccccc4)cc3)c3cccc(-c4ccc5c(c4)c(-c4ccccc4)c(-c4ccccc4)c4ccccc45)c3)c21 ; CC1(c2ccccc2)c2ccccc2-c2ccc(N(c3ccc(-c4ccccc4)cc3)c3cccc(-c4ccc5c(c4)c(-c4ccccc4)c(-c4ccccc4)c4ccccc45)c3)cc21]

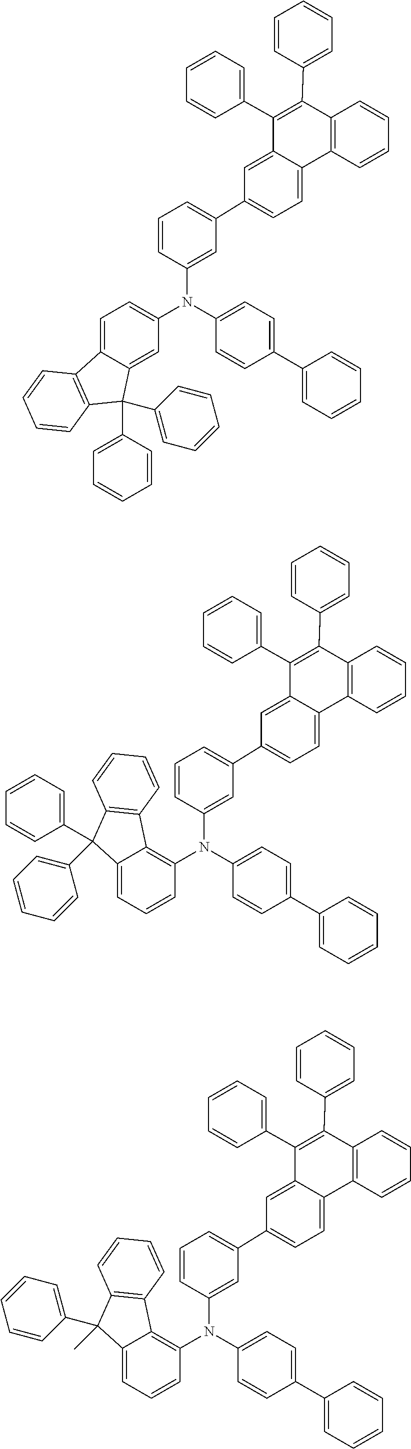 [SMILES: c1ccc(-c2ccc(N(c3cccc(-c4ccc5c(c4)c(-c4ccccc4)c(-c4ccccc4)c4ccccc45)c3)c3ccc4c(c3)C(c3ccccc3)(c3ccccc3)c3ccccc3-4)cc2)cc1 ; c1ccc(-c2ccc(N(c3cccc(-c4ccc5c(c4)c(-c4ccccc4)c(-c4ccccc4)c4ccccc45)c3)c3cccc4c3-c3ccccc3C4(c3ccccc3)c3ccccc3)cc2)cc1 ; CC1(c2ccccc2)c2ccccc2-c2c(N(c3ccc(-c4ccccc4)cc3)c3cccc(-c4ccc5c(c4)c(-c4ccccc4)c(-c4ccccc4)c4ccccc45)c3)cccc21]

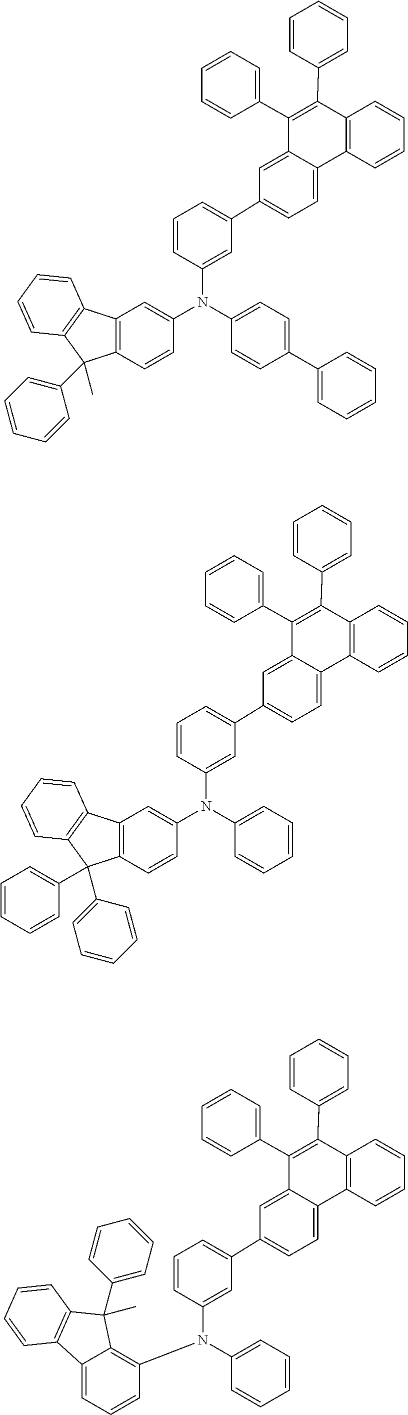 [SMILES: CC1(c2ccccc2)c2ccccc2-c2cc(N(c3ccc(-c4ccccc4)cc3)c3cccc(-c4ccc5c(c4)c(-c4ccccc4)c(-c4ccccc4)c4ccccc45)c3)ccc21 ; c1ccc(-c2c(-c3ccccc3)c3cc(-c4cccc(N(c5ccccc5)c5ccc6c(c5)-c5ccccc5C6(c5ccccc5)c5ccccc5)c4)ccc3c3ccccc23)cc1 ; CC1(c2ccccc2)c2ccccc2-c2cccc(N(c3ccccc3)c3cccc(-c4ccc5c(c4)c(-c4ccccc4)c(-c4ccccc4)c4ccccc45)c3)c21]

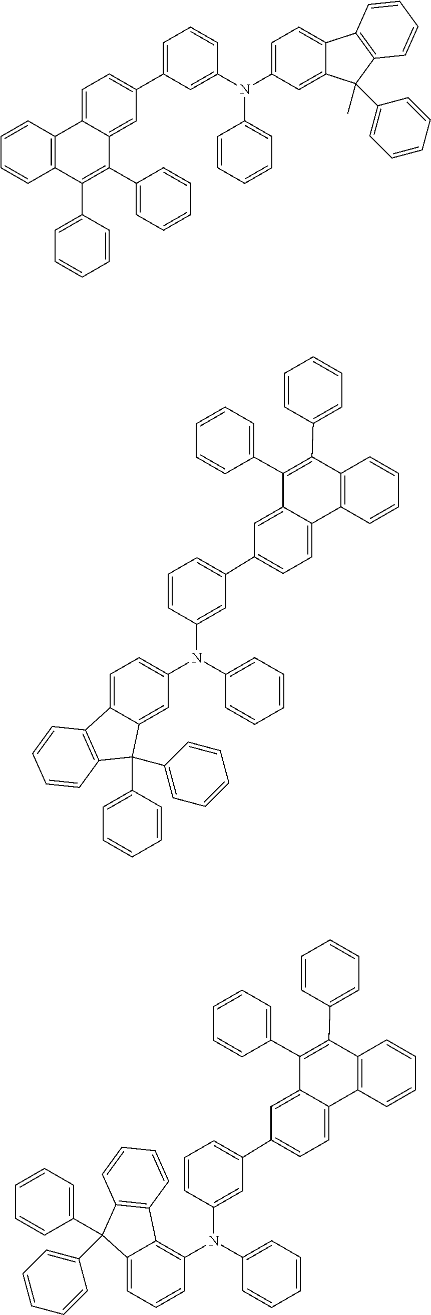 [SMILES: CC1(c2ccccc2)c2ccccc2-c2ccc(N(c3ccccc3)c3cccc(-c4ccc5c(c4)c(-c4ccccc4)c(-c4ccccc4)c4ccccc45)c3)cc21 ; c1ccc(-c2c(-c3ccccc3)c3cc(-c4cccc(N(c5ccccc5)c5ccc6c(c5)C(c5ccccc5)(c5ccccc5)c5ccccc5-6)c4)ccc3c3ccccc23)cc1 ; c1ccc(-c2c(-c3ccccc3)c3cc(-c4cccc(N(c5ccccc5)c5cccc6c5-c5ccccc5C6(c5ccccc5)c5ccccc5)c4)ccc3c3ccccc23)cc1]

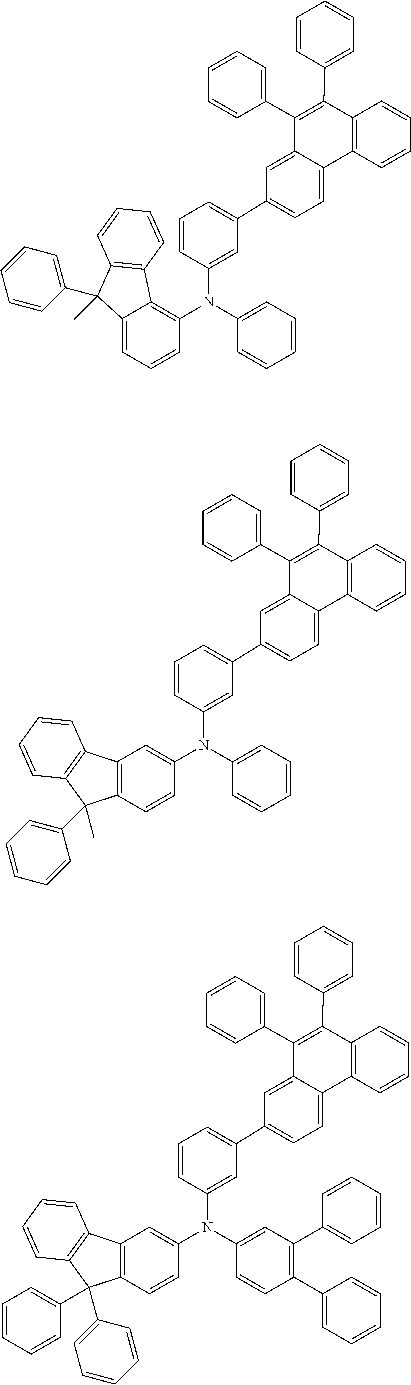 [SMILES: CC1(c2ccccc2)c2ccccc2-c2c(N(c3ccccc3)c3cccc(-c4ccc5c(c4)c(-c4ccccc4)c(-c4ccccc4)c4ccccc45)c3)cccc21 ; CC1(c2ccccc2)c2ccccc2-c2cc(N(c3ccccc3)c3cccc(-c4ccc5c(c4)c(-c4ccccc4)c(-c4ccccc4)c4ccccc45)c3)ccc21 ; c1ccc(-c2ccc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)cc2-c2ccccc2)cc1]

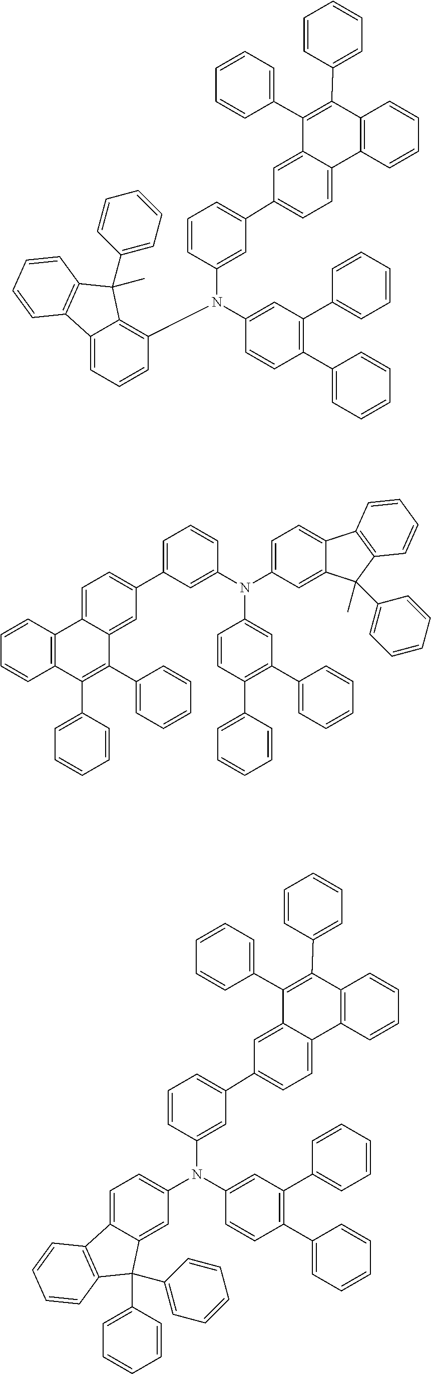 [SMILES: CC1(c2ccccc2)c2ccccc2-c2cccc(N(c3cccc(-c4ccc5c(c4)c(-c4ccccc4)c(-c4ccccc4)c4ccccc45)c3)c3ccc(-c4ccccc4)c(-c4ccccc4)c3)c21 ; CC1(c2ccccc2)c2ccccc2-c2ccc(N(c3cccc(-c4ccc5c(c4)c(-c4ccccc4)c(-c4ccccc4)c4ccccc45)c3)c3ccc(-c4ccccc4)c(-c4ccccc4)c3)cc21 ; c1ccc(-c2ccc(N(c3cccc(-c4ccc5c(c4)c(-c4ccccc4)c(-c4ccccc4)c4ccccc45)c3)c3ccc4c(c3)C(c3ccccc3)(c3ccccc3)c3ccccc3-4)cc2-c2ccccc2)cc1]

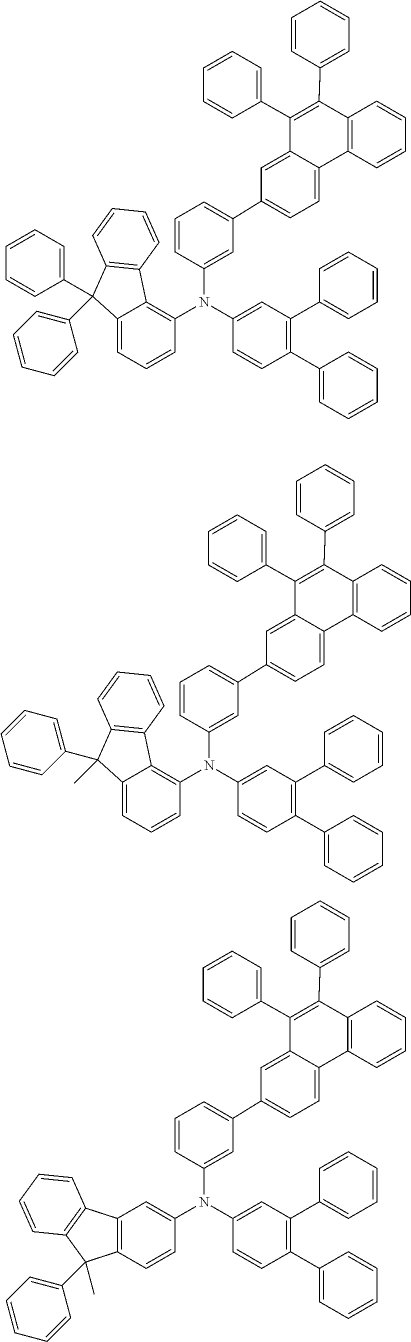

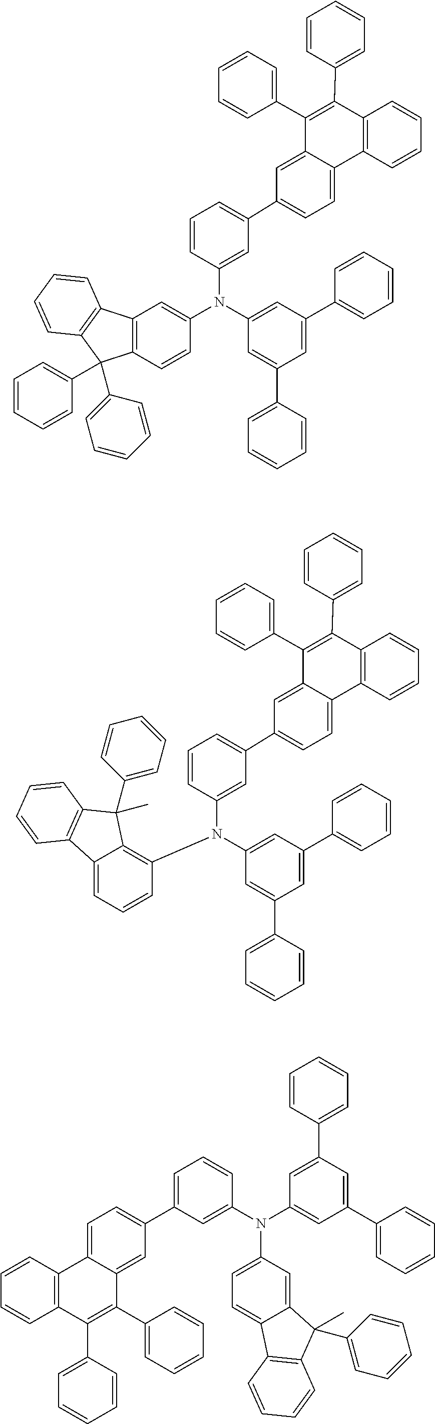 [SMILES: c1ccc(-c2cc(-c3ccccc3)cc(N(c3cccc(-c4ccc5c(c4)c(-c4ccccc4)c(-c4ccccc4)c4ccccc45)c3)c3ccc4c(c3)-c3ccccc3C4(c3ccccc3)c3ccccc3)c2)cc1 ; CC1(c2ccccc2)c2ccccc2-c2cccc(N(c3cccc(-c4ccc5c(c4)c(-c4ccccc4)c(-c4ccccc4)c4ccccc45)c3)c3cc(-c4ccccc4)cc(-c4ccccc4)c3)c21 ; CC1(c2ccccc2)c2ccccc2-c2ccc(N(c3cccc(-c4ccc5c(c4)c(-c4ccccc4)c(-c4ccccc4)c4ccccc45)c3)c3cc(-c4ccccc4)cc(-c4ccccc4)c3)cc21]

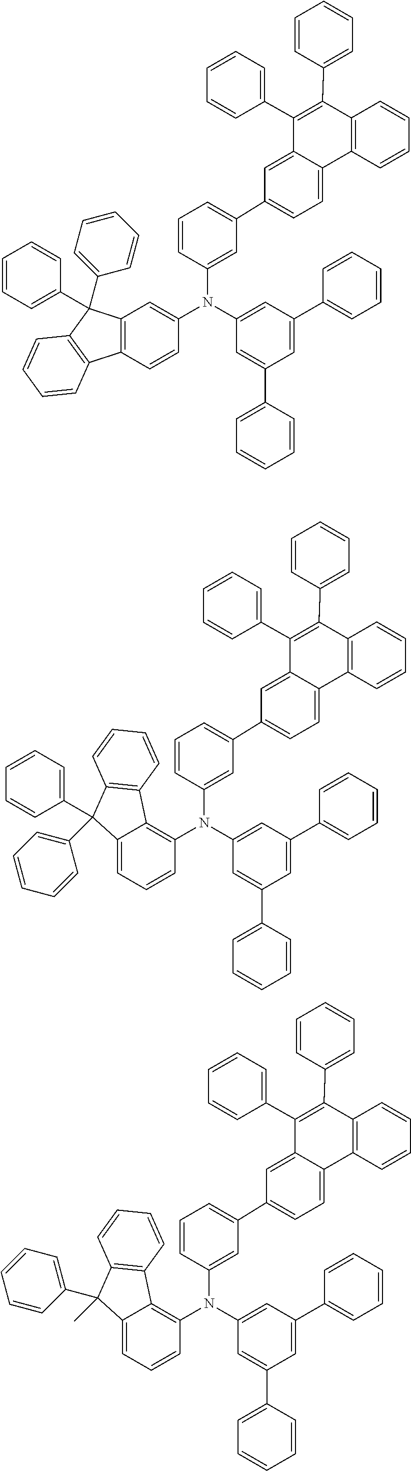

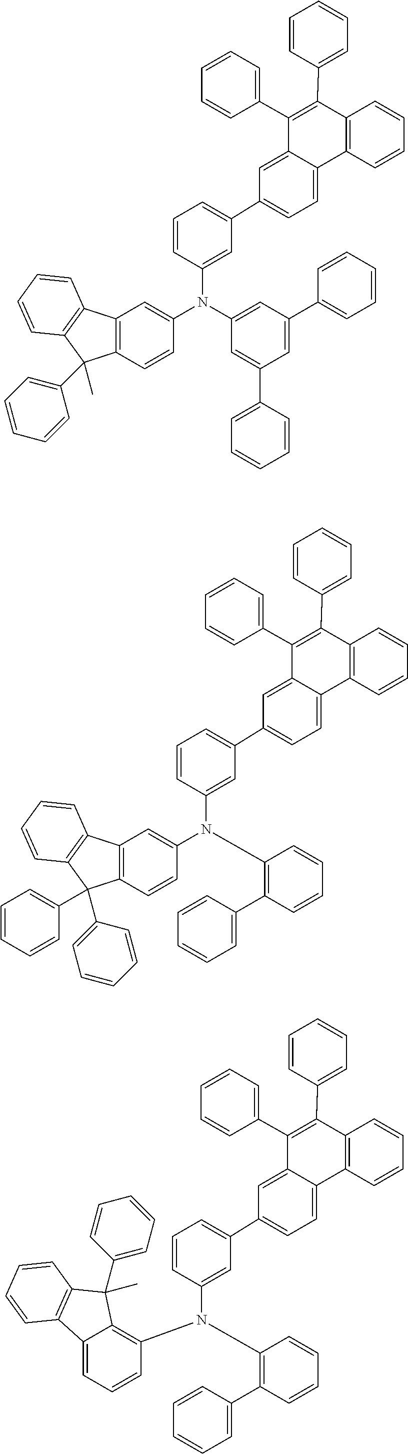 [SMILES: CC1(c2ccccc2)c2ccccc2-c2cc(N(c3cccc(-c4ccc5c(c4)c(-c4ccccc4)c(-c4ccccc4)c4ccccc45)c3)c3cc(-c4ccccc4)cc(-c4ccccc4)c3)ccc21 ; c1ccc(-c2ccccc2N(c2cccc(-c3ccc4c(c3)c(-c3ccccc3)c(-c3ccccc3)c3ccccc34)c2)c2ccc3c(c2)-c2ccccc2C3(c2ccccc2)c2ccccc2)cc1 ; CC1(c2ccccc2)c2ccccc2-c2cccc(N(c3cccc(-c4ccc5c(c4)c(-c4ccccc4)c(-c4ccccc4)c4ccccc45)c3)c3ccccc3-c3ccccc3)c21]

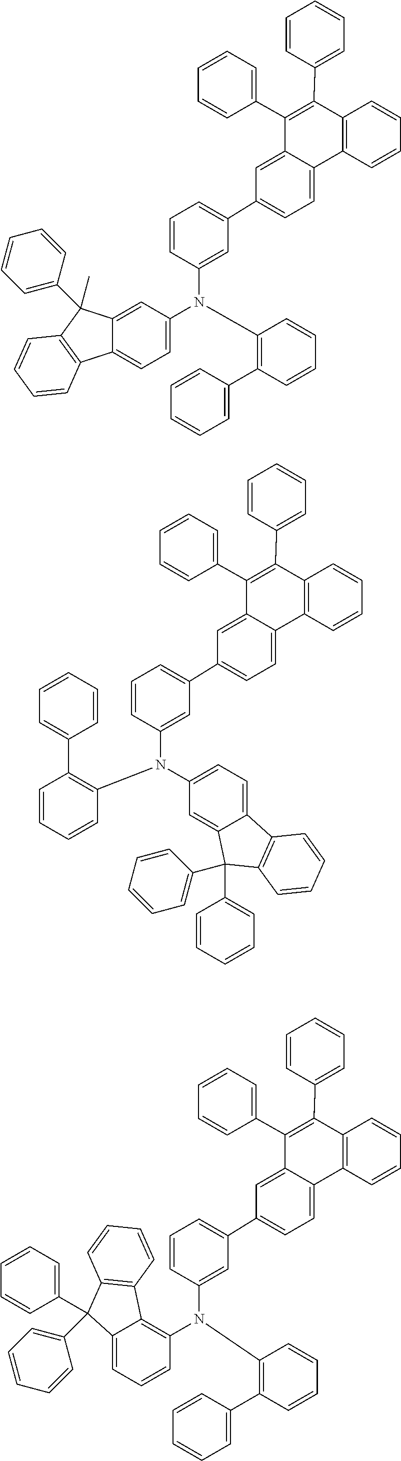 [SMILES: CC1(c2ccccc2)c2ccccc2-c2ccc(N(c3cccc(-c4ccc5c(c4)c(-c4ccccc4)c(-c4ccccc4)c4ccccc45)c3)c3ccccc3-c3ccccc3)cc21 ; c1ccc(-c2ccccc2N(c2cccc(-c3ccc4c(c3)c(-c3ccccc3)c(-c3ccccc3)c3ccccc34)c2)c2ccc3c(c2)C(c2ccccc2)(c2ccccc2)c2ccccc2-3)cc1 ; c1ccc(-c2ccccc2N(c2cccc(-c3ccc4c(c3)c(-c3ccccc3)c(-c3ccccc3)c3ccccc34)c2)c2cccc3c2-c2ccccc2C3(c2ccccc2)c2ccccc2)cc1]

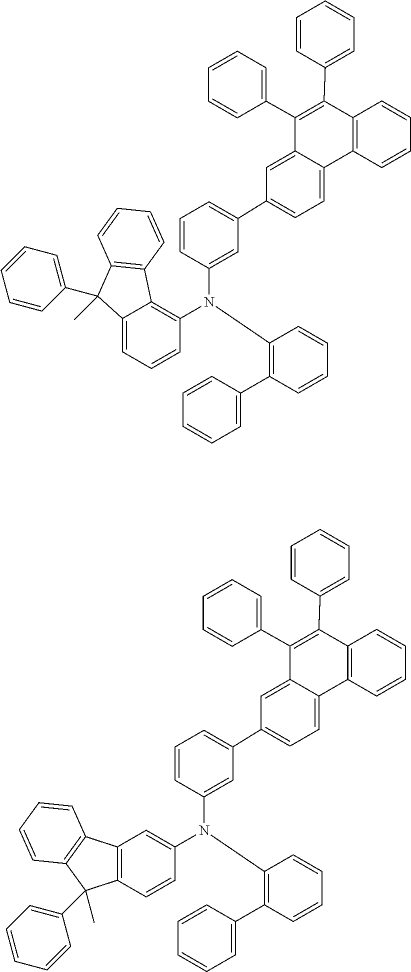 [SMILES: CC1(c2ccccc2)c2ccccc2-c2c(N(c3cccc(-c4ccc5c(c4)c(-c4ccccc4)c(-c4ccccc4)c4ccccc45)c3)c3ccccc3-c3ccccc3)cccc21 ; CC1(c2ccccc2)c2ccccc2-c2cc(N(c3cccc(-c4ccc5c(c4)c(-c4ccccc4)c(-c4ccccc4)c4ccccc45)c3)c3ccccc3-c3ccccc3)ccc21]

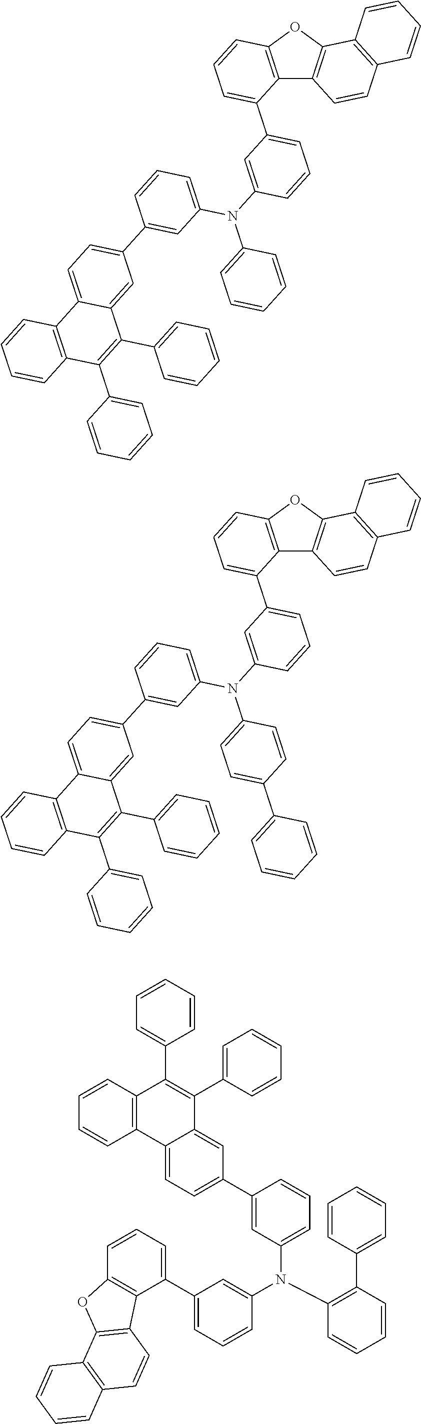 [SMILES: c1ccc(-c2c(-c3ccccc3)c3cc(-c4cccc(N(c5ccccc5)c5cccc(-c6cccc7oc8c9ccccc9ccc8c67)c5)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc(-c4cccc5oc6c7ccccc7ccc6c45)c3)cc2)cc1 ; c1ccc(-c2ccccc2N(c2cccc(-c3ccc4c(c3)c(-c3ccccc3)c(-c3ccccc3)c3ccccc34)c2)c2cccc(-c3cccc4oc5c6ccccc6ccc5c34)c2)cc1]

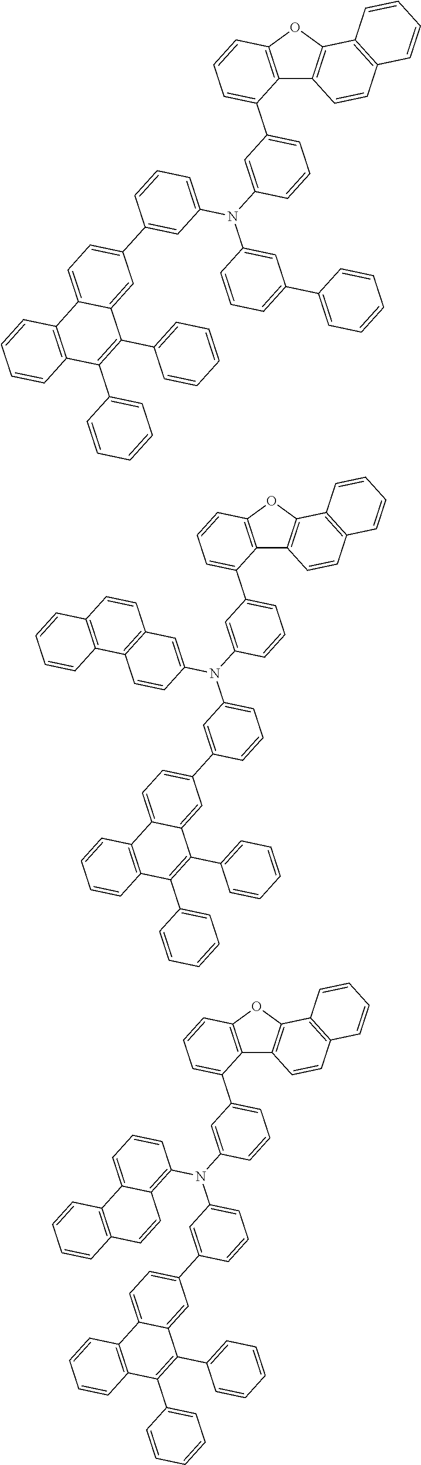 [SMILES: c1ccc(-c2cccc(N(c3cccc(-c4ccc5c(c4)c(-c4ccccc4)c(-c4ccccc4)c4ccccc45)c3)c3cccc(-c4cccc5oc6c7ccccc7ccc6c45)c3)c2)cc1 ; c1ccc(-c2c(-c3ccccc3)c3cc(-c4cccc(N(c5cccc(-c6cccc7oc8c9ccccc9ccc8c67)c5)c5ccc6c(ccc7ccccc76)c5)c4)ccc3c3ccccc23)cc1 ; c1ccc(-c2c(-c3ccccc3)c3cc(-c4cccc(N(c5cccc(-c6cccc7oc8c9ccccc9ccc8c67)c5)c5cccc6c5ccc5ccccc56)c4)ccc3c3ccccc23)cc1]

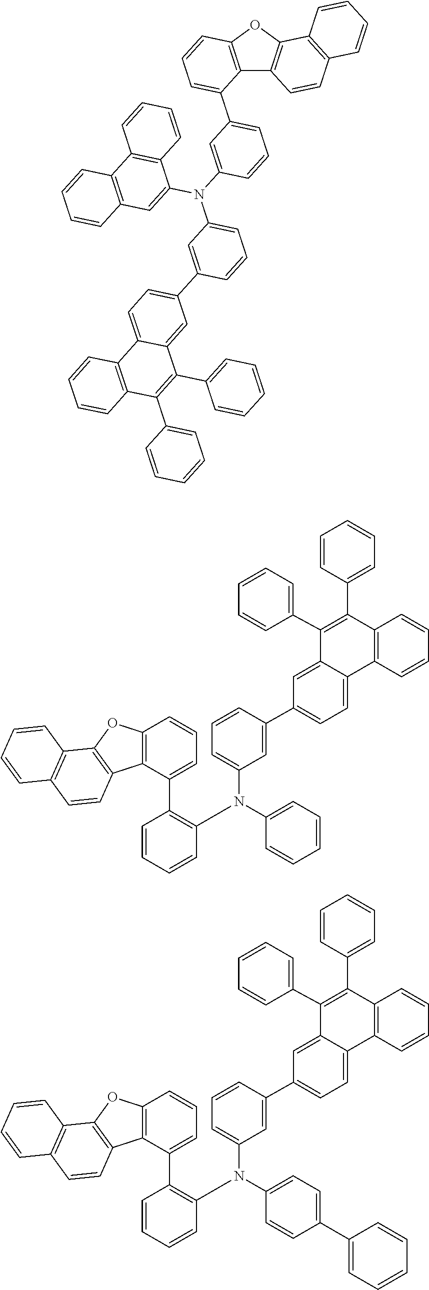 [SMILES: c1ccc(-c2c(-c3ccccc3)c3cc(-c4cccc(N(c5cccc(-c6cccc7oc8c9ccccc9ccc8c67)c5)c5cc6ccccc6c6ccccc56)c4)ccc3c3ccccc23)cc1 ; c1ccc(-c2c(-c3ccccc3)c3cc(-c4cccc(N(c5ccccc5)c5ccccc5-c5cccc6oc7c8ccccc8ccc7c56)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3ccccc3-c3cccc4oc5c6ccccc6ccc5c34)cc2)cc1]

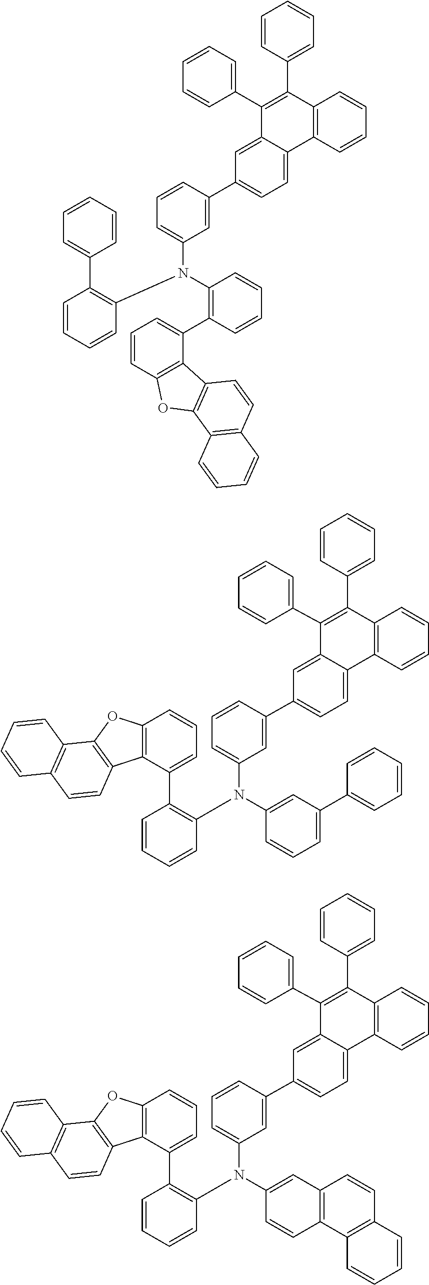 [SMILES: c1ccc(-c2ccccc2N(c2cccc(-c3ccc4c(c3)c(-c3ccccc3)c(-c3ccccc3)c3ccccc34)c2)c2ccccc2-c2cccc3oc4c5ccccc5ccc4c23)cc1 ; c1ccc(-c2cccc(N(c3cccc(-c4ccc5c(c4)c(-c4ccccc4)c(-c4ccccc4)c4ccccc45)c3)c3ccccc3-c3cccc4oc5c6ccccc6ccc5c34)c2)cc1 ; c1ccc(-c2c(-c3ccccc3)c3cc(-c4cccc(N(c5ccc6c(ccc7ccccc76)c5)c5ccccc5-c5cccc6oc7c8ccccc8ccc7c56)c4)ccc3c3ccccc23)cc1]

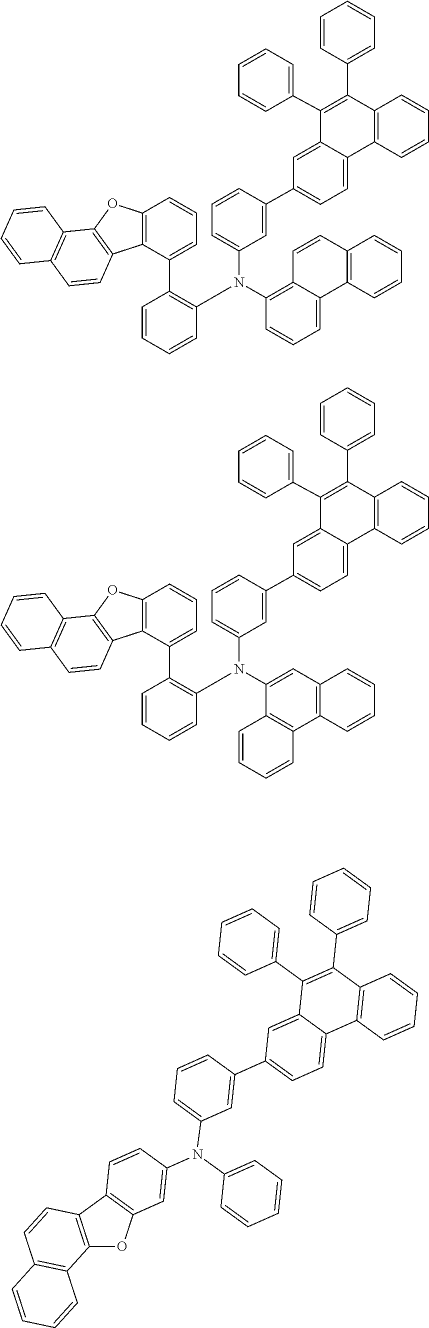 [SMILES: c1ccc(-c2c(-c3ccccc3)c3cc(-c4cccc(N(c5ccccc5-c5cccc6oc7c8ccccc8ccc7c56)c5cccc6c5ccc5ccccc56)c4)ccc3c3ccccc23)cc1 ; c1ccc(-c2c(-c3ccccc3)c3cc(-c4cccc(N(c5ccccc5-c5cccc6oc7c8ccccc8ccc7c56)c5cc6ccccc6c6ccccc56)c4)ccc3c3ccccc23)cc1 ; c1ccc(-c2c(-c3ccccc3)c3cc(-c4cccc(N(c5ccccc5)c5ccc6c(c5)oc5c7ccccc7ccc65)c4)ccc3c3ccccc23)cc1]

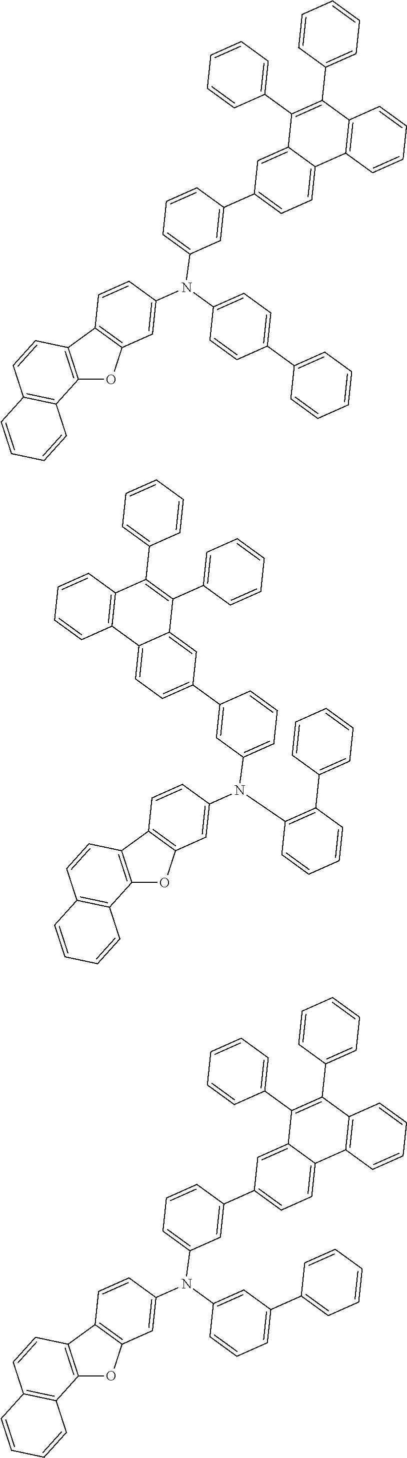 [SMILES: c1ccc(-c2ccc(N(c3cccc(-c4ccc5c(c4)c(-c4ccccc4)c(-c4ccccc4)c4ccccc45)c3)c3ccc4c(c3)oc3c5ccccc5ccc43)cc2)cc1 ; c1ccc(-c2ccccc2N(c2cccc(-c3ccc4c(c3)c(-c3ccccc3)c(-c3ccccc3)c3ccccc34)c2)c2ccc3c(c2)oc2c4ccccc4ccc32)cc1 ; c1ccc(-c2cccc(N(c3cccc(-c4ccc5c(c4)c(-c4ccccc4)c(-c4ccccc4)c4ccccc45)c3)c3ccc4c(c3)oc3c5ccccc5ccc43)c2)cc1]

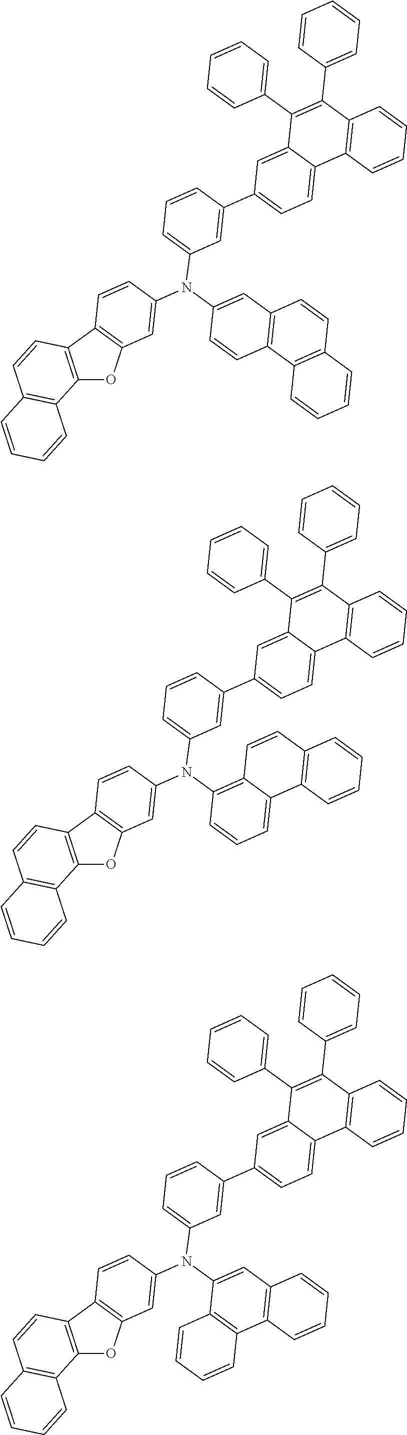 [SMILES: c1ccc(-c2c(-c3ccccc3)c3cc(-c4cccc(N(c5ccc6c(ccc7ccccc76)c5)c5ccc6c(c5)oc5c7ccccc7ccc65)c4)ccc3c3ccccc23)cc1 ; c1ccc(-c2c(-c3ccccc3)c3cc(-c4cccc(N(c5ccc6c(c5)oc5c7ccccc7ccc65)c5cccc6c5ccc5ccccc56)c4)ccc3c3ccccc23)cc1 ; c1ccc(-c2c(-c3ccccc3)c3cc(-c4cccc(N(c5ccc6c(c5)oc5c7ccccc7ccc65)c5cc6ccccc6c6ccccc56)c4)ccc3c3ccccc23)cc1]

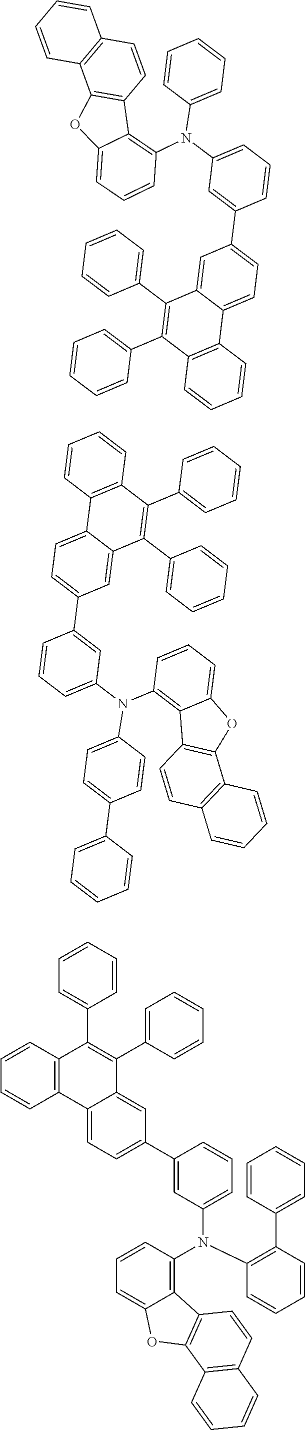

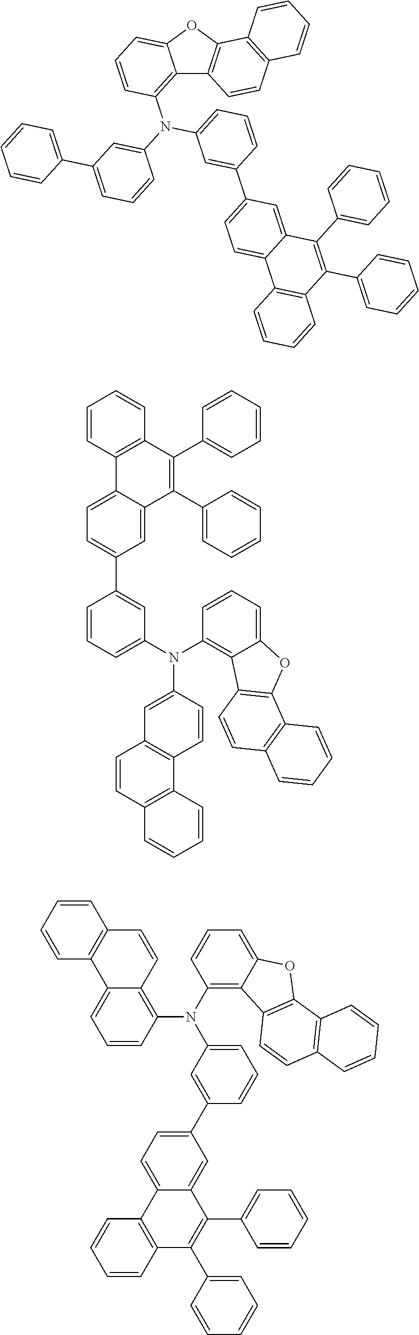 [SMILES: c1ccc(-c2cccc(N(c3cccc(-c4ccc5c(c4)c(-c4ccccc4)c(-c4ccccc4)c4ccccc45)c3)c3cccc4oc5c6ccccc6ccc5c34)c2)cc1 ; c1ccc(-c2c(-c3ccccc3)c3cc(-c4cccc(N(c5ccc6c(ccc7ccccc76)c5)c5cccc6oc7c8ccccc8ccc7c56)c4)ccc3c3ccccc23)cc1 ; c1ccc(-c2c(-c3ccccc3)c3cc(-c4cccc(N(c5cccc6c5ccc5ccccc56)c5cccc6oc7c8ccccc8ccc7c56)c4)ccc3c3ccccc23)cc1]

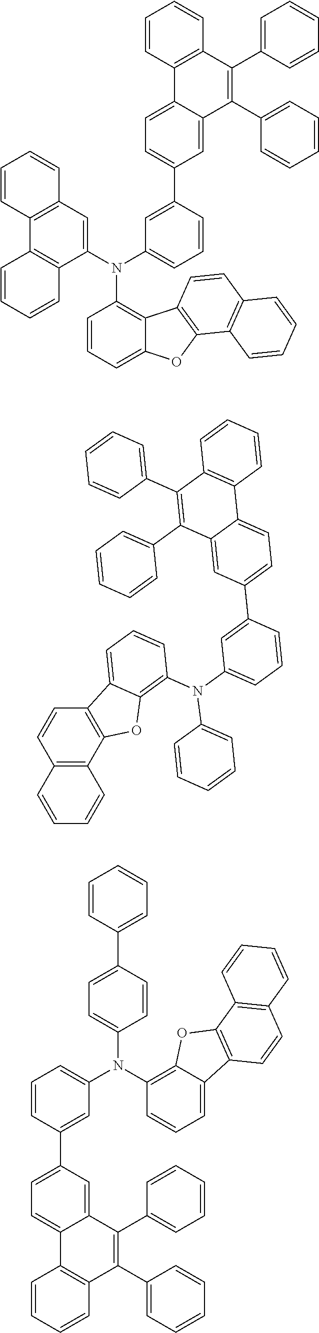 [SMILES: c1ccc(-c2c(-c3ccccc3)c3cc(-c4cccc(N(c5cc6ccccc6c6ccccc56)c5cccc6oc7c8ccccc8ccc7c56)c4)ccc3c3ccccc23)cc1 ; c1ccc(-c2c(-c3ccccc3)c3cc(-c4cccc(N(c5ccccc5)c5cccc6c5oc5c7ccccc7ccc65)c4)ccc3c3ccccc23)cc1 ; c1ccc(-c2ccc(N(c3cccc(-c4ccc5c(c4)c(-c4ccccc4)c(-c4ccccc4)c4ccccc45)c3)c3cccc4c3oc3c5ccccc5ccc43)cc2)cc1]

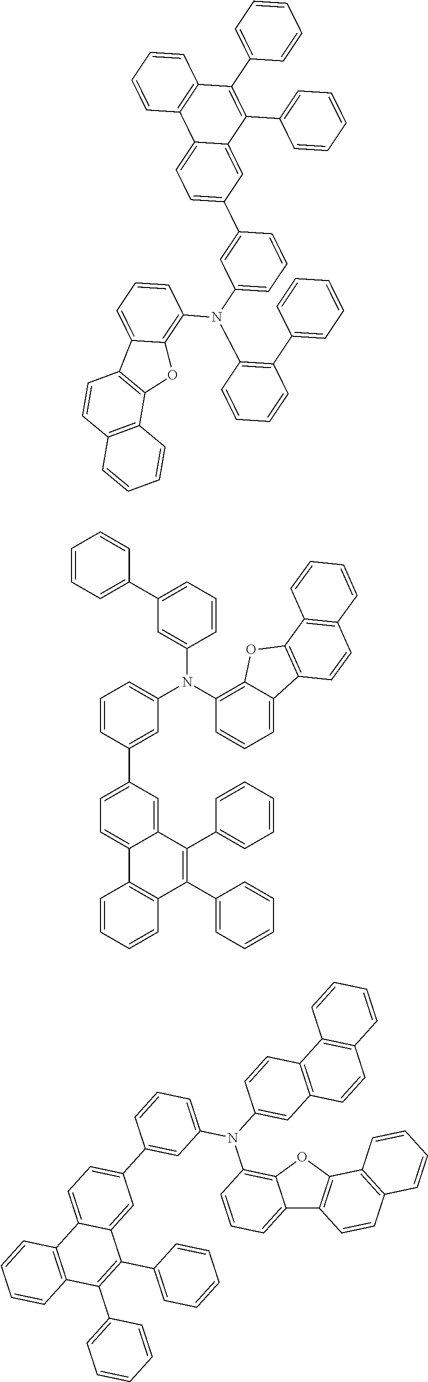 [SMILES: c1ccc(-c2ccccc2N(c2cccc(-c3ccc4c(c3)c(-c3ccccc3)c(-c3ccccc3)c3ccccc34)c2)c2cccc3c2oc2c4ccccc4ccc32)cc1 ; c1ccc(-c2cccc(N(c3cccc(-c4ccc5c(c4)c(-c4ccccc4)c(-c4ccccc4)c4ccccc45)c3)c3cccc4c3oc3c5ccccc5ccc43)c2)cc1 ; c1ccc(-c2c(-c3ccccc3)c3cc(-c4cccc(N(c5ccc6c(ccc7ccccc76)c5)c5cccc6c5oc5c7ccccc7ccc65)c4)ccc3c3ccccc23)cc1]

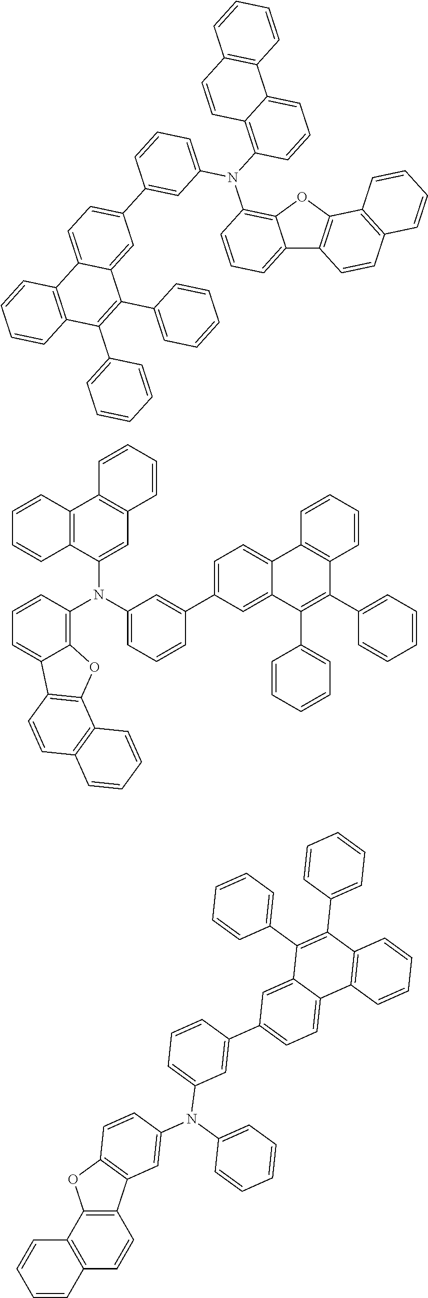

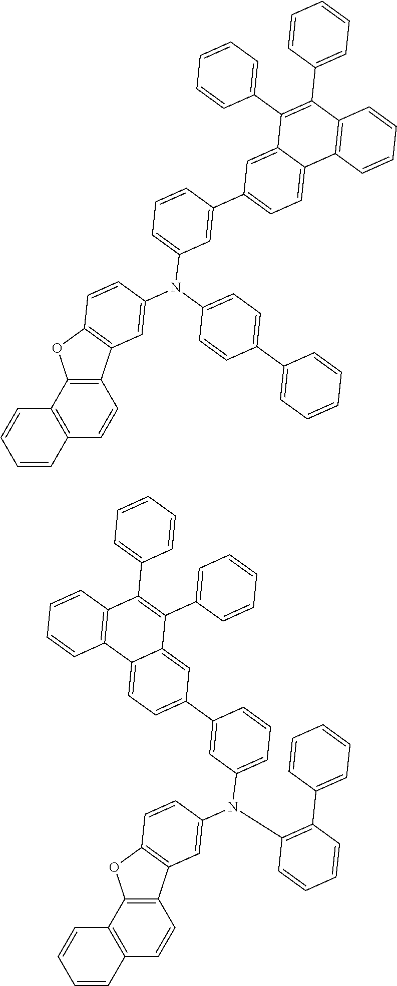 [SMILES: c1ccc(-c2ccc(N(c3cccc(-c4ccc5c(c4)c(-c4ccccc4)c(-c4ccccc4)c4ccccc45)c3)c3ccc4oc5c6ccccc6ccc5c4c3)cc2)cc1 ; c1ccc(-c2ccccc2N(c2cccc(-c3ccc4c(c3)c(-c3ccccc3)c(-c3ccccc3)c3ccccc34)c2)c2ccc3oc4c5ccccc5ccc4c3c2)cc1]

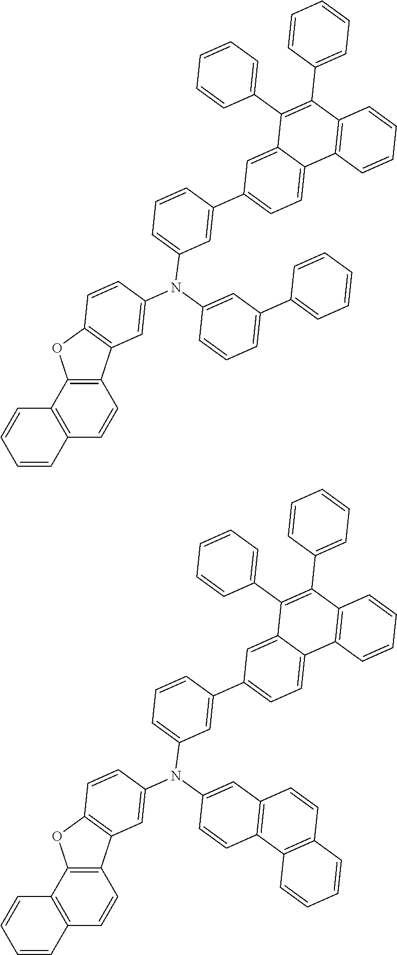 [SMILES: c1ccc(-c2cccc(N(c3cccc(-c4ccc5c(c4)c(-c4ccccc4)c(-c4ccccc4)c4ccccc45)c3)c3ccc4oc5c6ccccc6ccc5c4c3)c2)cc1 ; c1ccc(-c2c(-c3ccccc3)c3cc(-c4cccc(N(c5ccc6c(ccc7ccccc76)c5)c5ccc6oc7c8ccccc8ccc7c6c5)c4)ccc3c3ccccc23)cc1]

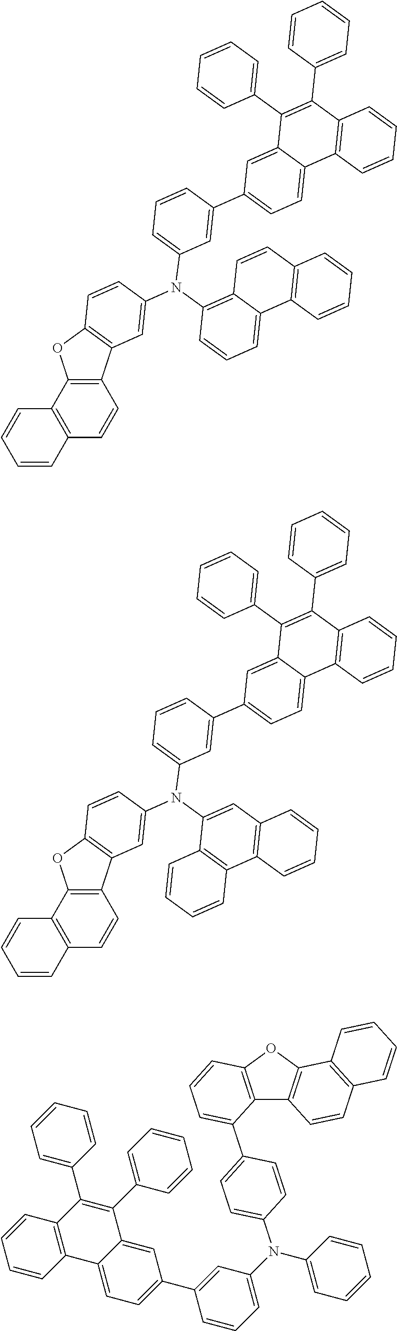 [SMILES: c1ccc(-c2c(-c3ccccc3)c3cc(-c4cccc(N(c5ccc6oc7c8ccccc8ccc7c6c5)c5cccc6c5ccc5ccccc56)c4)ccc3c3ccccc23)cc1 ; c1ccc(-c2c(-c3ccccc3)c3cc(-c4cccc(N(c5ccc6oc7c8ccccc8ccc7c6c5)c5cc6ccccc6c6ccccc56)c4)ccc3c3ccccc23)cc1 ; c1ccc(-c2c(-c3ccccc3)c3cc(-c4cccc(N(c5ccccc5)c5ccc(-c6cccc7oc8c9ccccc9ccc8c67)cc5)c4)ccc3c3ccccc23)cc1]

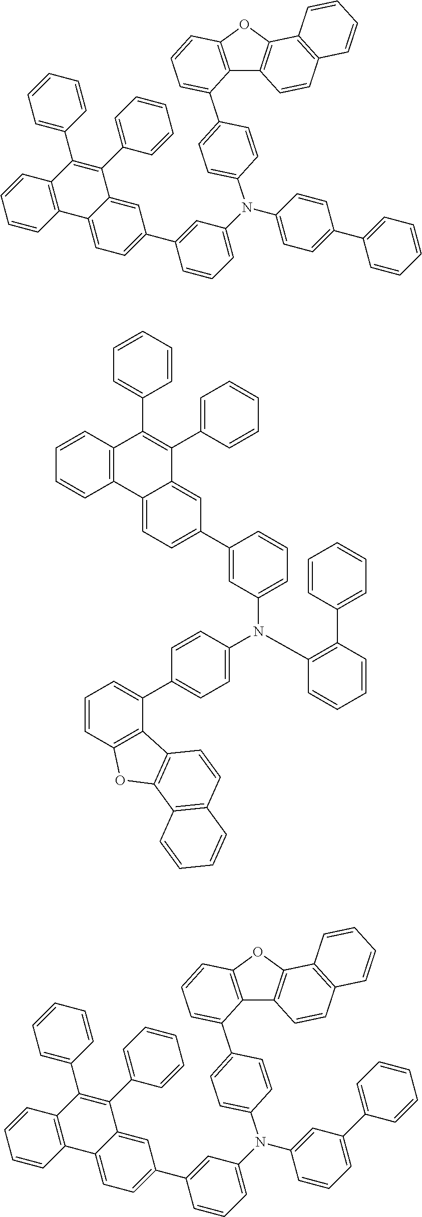 [SMILES: c1ccc(-c2ccc(N(c3ccc(-c4cccc5oc6c7ccccc7ccc6c45)cc3)c3cccc(-c4ccc5c(c4)c(-c4ccccc4)c(-c4ccccc4)c4ccccc45)c3)cc2)cc1 ; c1ccc(-c2ccccc2N(c2ccc(-c3cccc4oc5c6ccccc6ccc5c34)cc2)c2cccc(-c3ccc4c(c3)c(-c3ccccc3)c(-c3ccccc3)c3ccccc34)c2)cc1 ; c1ccc(-c2cccc(N(c3ccc(-c4cccc5oc6c7ccccc7ccc6c45)cc3)c3cccc(-c4ccc5c(c4)c(-c4ccccc4)c(-c4ccccc4)c4ccccc45)c3)c2)cc1]

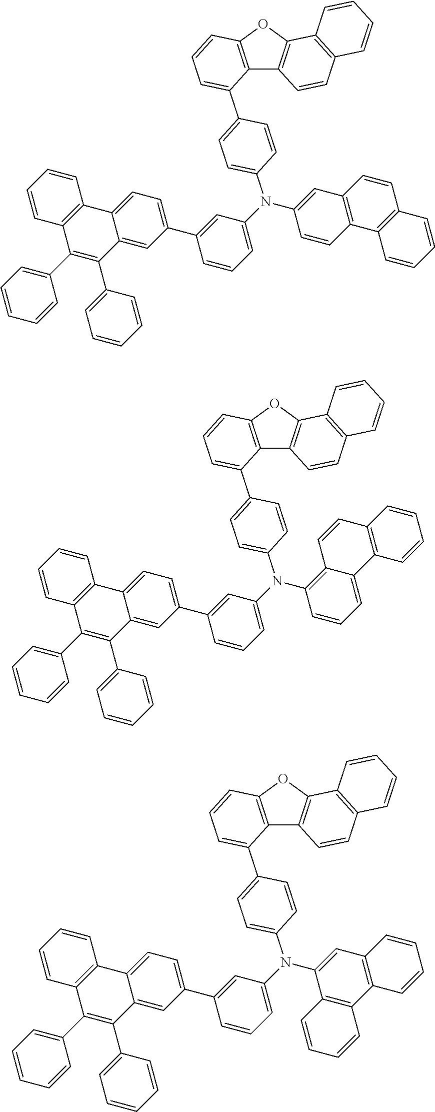 [SMILES: c1ccc(-c2c(-c3ccccc3)c3cc(-c4cccc(N(c5ccc(-c6cccc7oc8c9ccccc9ccc8c67)cc5)c5ccc6c(ccc7ccccc76)c5)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5cccc6c5ccc5ccccc56)c4)ccc3c3ccccc23)cc1 ; c1ccc(-c2c(-c3ccccc3)c3cc(-c4cccc(N(c5ccc(-c6cccc7oc8c9ccccc9ccc8c67)cc5)c5cc6ccccc6c6ccccc56)c4)ccc3c3ccccc23)cc1]

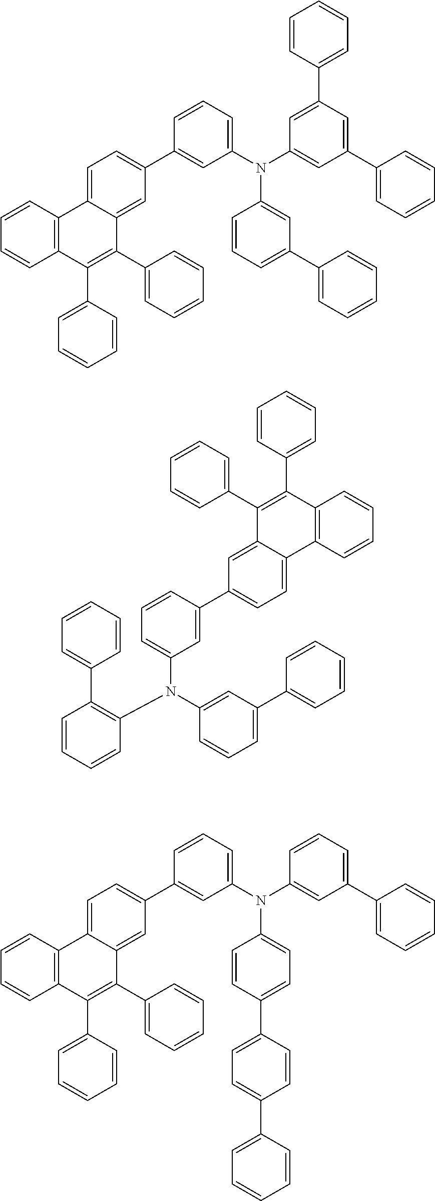 [SMILES: c1ccc(-c2cccc(N(c3cccc(-c4ccc5c(c4)c(-c4ccccc4)c(-c4ccccc4)c4ccccc45)c3)c3cc(-c4ccccc4)cc(-c4ccccc4)c3)c2)cc1 ; c1ccc(-c2cccc(N(c3cccc(-c4ccc5c(c4)c(-c4ccccc4)c(-c4ccccc4)c4ccccc45)c3)c3ccccc3-c3ccccc3)c2)cc1 ; c1ccc(-c2ccc(-c3ccc(N(c4cccc(-c5ccccc5)c4)c4cccc(-c5ccc6c(c5)c(-c5ccccc5)c(-c5ccccc5)c5ccccc56)c4)cc3)cc2)cc1]

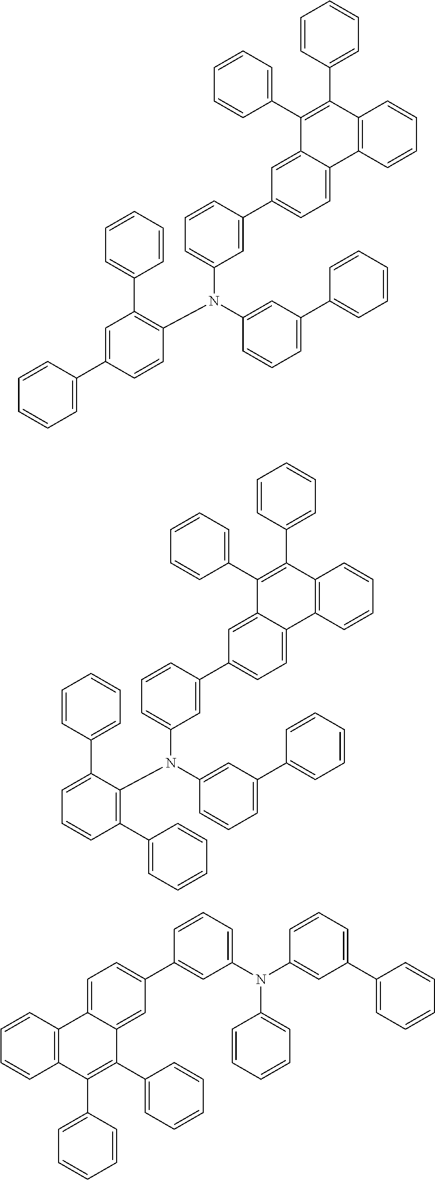

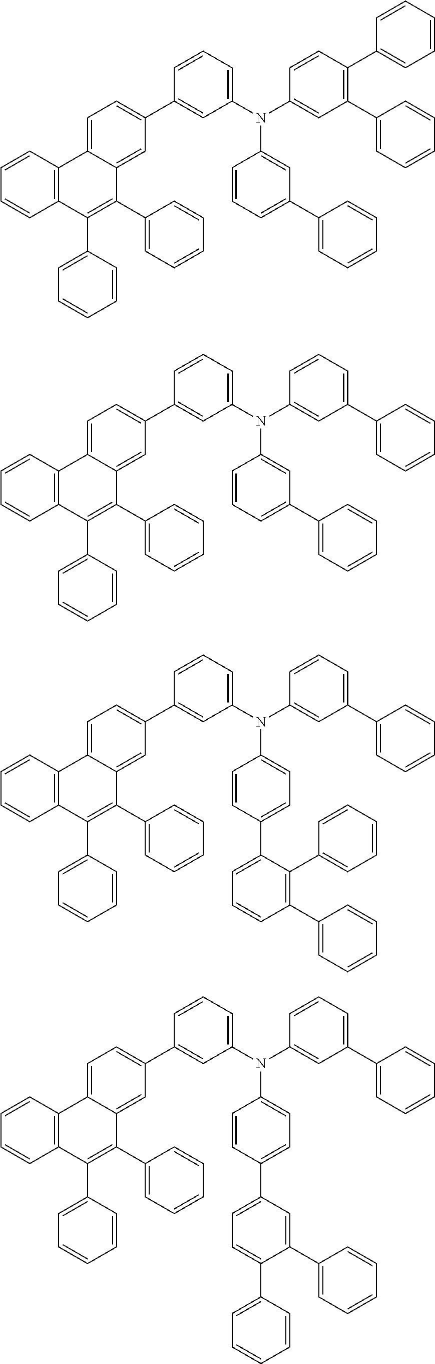

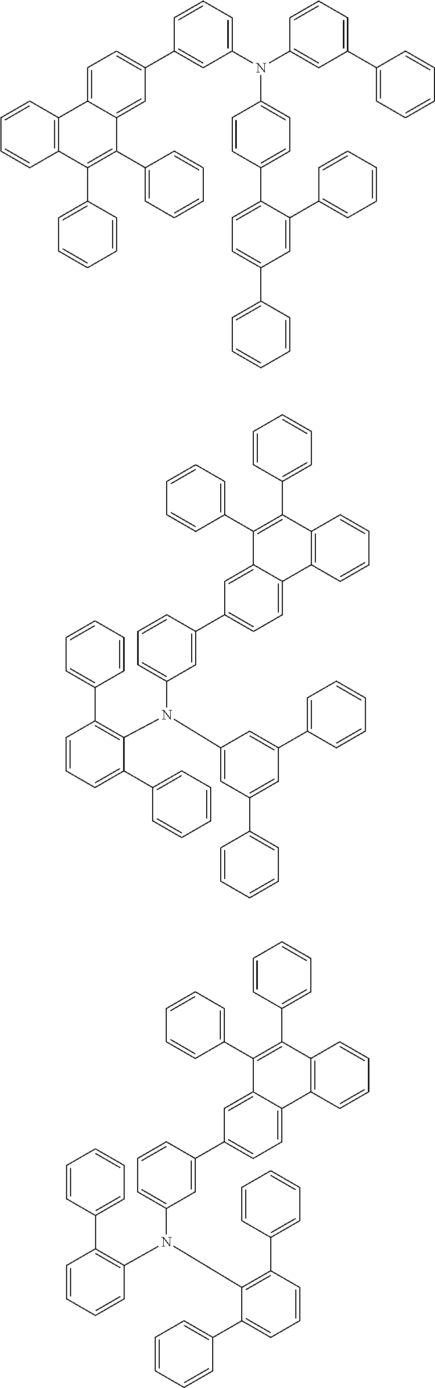 [SMILES: c1ccc(-c2cccc(N(c3ccc(-c4ccc(-c5ccccc5)cc4-c4ccccc4)cc3)c3cccc(-c4ccc5c(c4)c(-c4ccccc4)c(-c4ccccc4)c4ccccc45)c3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3c(-c4ccccc4)cccc3-c3ccccc3)c2)cc1 ; c1ccc(-c2ccccc2N(c2cccc(-c3ccc4c(c3)c(-c3ccccc3)c(-c3ccccc3)c3ccccc34)c2)c2c(-c3ccccc3)cccc2-c2ccccc2)cc1]

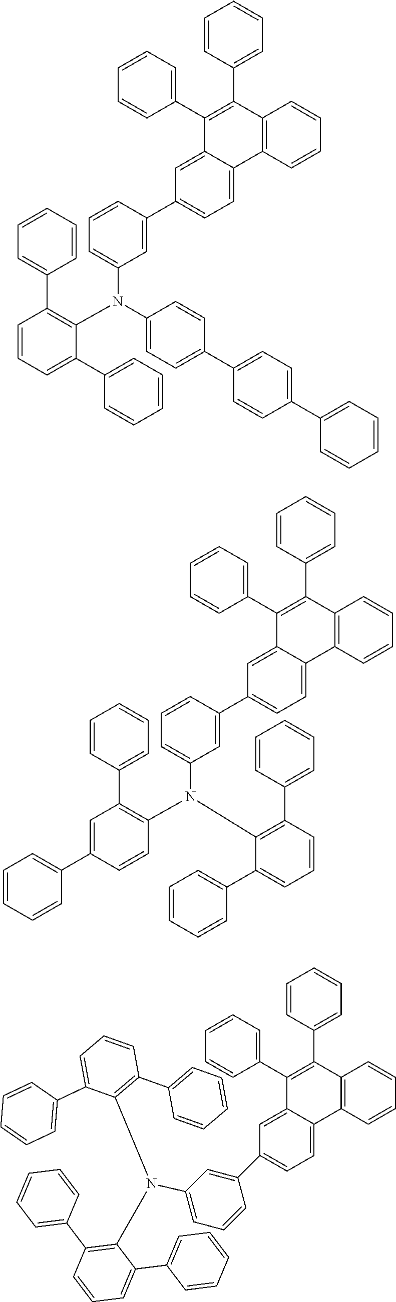 [SMILES: c1ccc(-c2ccc(-c3ccc(N(c4cccc(-c5ccc6c(c5)c(-c5ccccc5)c(-c5ccccc5)c5ccccc56)c4)c4c(-c5ccccc5)cccc4-c4ccccc4)cc3)cc2)cc1 ; c1ccc(-c2ccc(N(c3cccc(-c4ccc5c(c4)c(-c4ccccc4)c(-c4ccccc4)c4ccccc45)c3)c3c(-c4ccccc4)cccc3-c3ccccc3)c(-c3ccccc3)c2)cc1 ; c1ccc(-c2cccc(-c3ccccc3)c2N(c2cccc(-c3ccc4c(c3)c(-c3ccccc3)c(-c3ccccc3)c3ccccc34)c2)c2c(-c3ccccc3)cccc2-c2ccccc2)cc1]

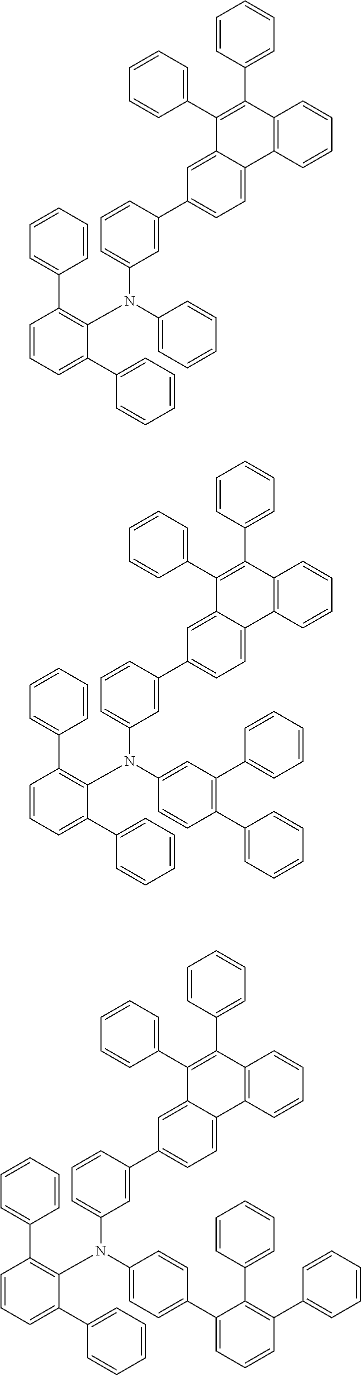 [SMILES: c1ccc(-c2cccc(-c3ccccc3)c2N(c2ccccc2)c2cccc(-c3ccc4c(c3)c(-c3ccccc3)c(-c3ccccc3)c3ccccc34)c2)cc1 ; c1ccc(-c2ccc(N(c3cccc(-c4ccc5c(c4)c(-c4ccccc4)c(-c4ccccc4)c4ccccc45)c3)c3c(-c4ccccc4)cccc3-c3ccccc3)cc2-c2ccccc2)cc1 ; c1ccc(-c2cccc(-c3ccc(N(c4cccc(-c5ccc6c(c5)c(-c5ccccc5)c(-c5ccccc5)c5ccccc56)c4)c4c(-c5ccccc5)cccc4-c4ccccc4)cc3)c2-c2ccccc2)cc1]

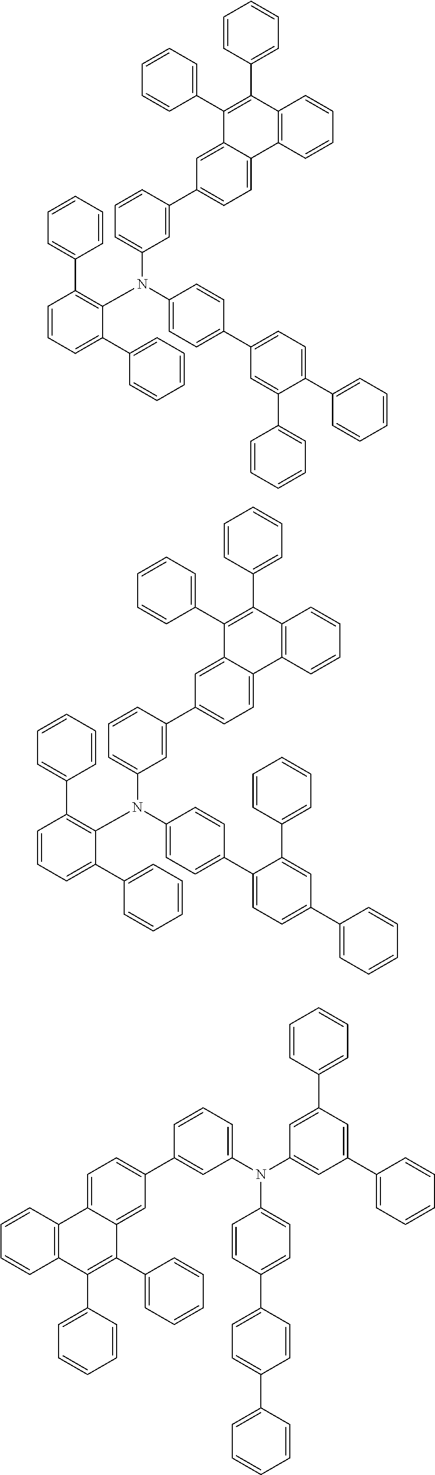

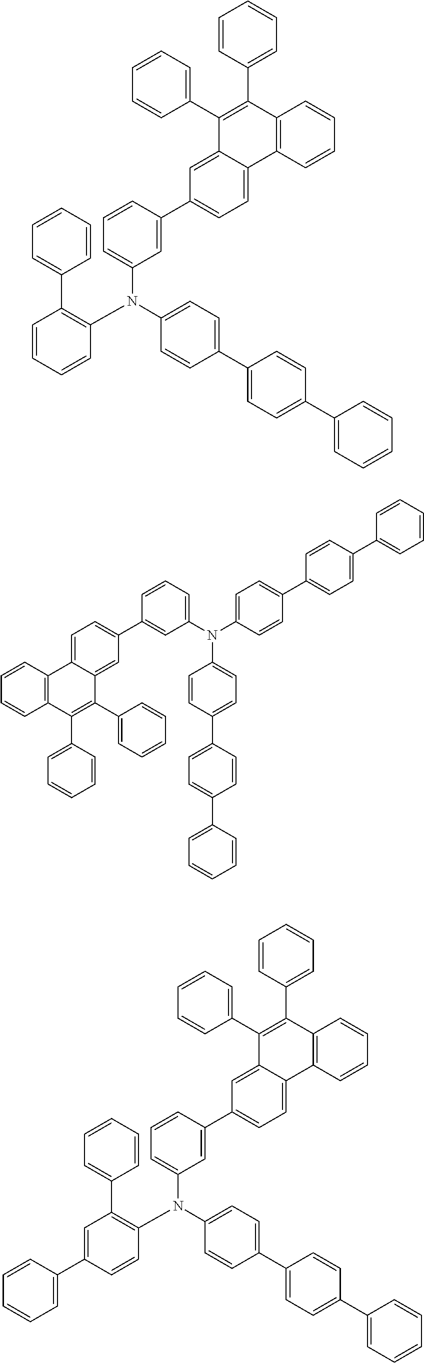 [SMILES: c1ccc(-c2ccc(-c3ccc(N(c4cccc(-c5ccc6c(c5)c(-c5ccccc5)c(-c5ccccc5)c5ccccc56)c4)c4ccccc4-c4ccccc4)cc3)cc2)cc1 ; c1ccc(-c2ccc(-c3ccc(N(c4ccc(-c5ccc(-c6ccccc6)cc5)cc4)c4cccc(-c5ccc6c(c5)c(-c5ccccc5)c(-c5ccccc5)c5ccccc56)c4)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4ccc(-c5ccccc5)cc4-c4ccccc4)cc3)cc2)cc1]

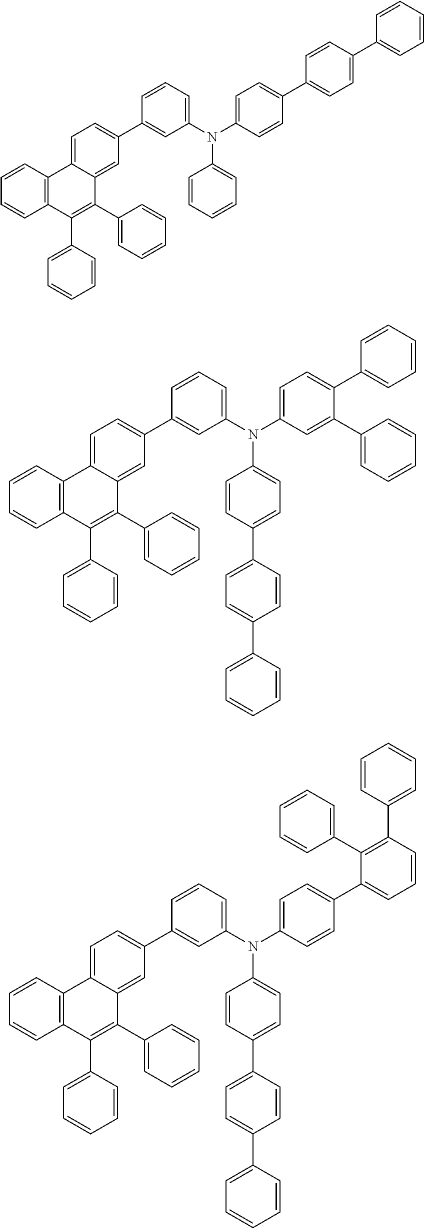 [SMILES: c1ccc(-c2ccc(-c3ccc(N(c4ccccc4)c4cccc(-c5ccc6c(c5)c(-c5ccccc5)c(-c5ccccc5)c5ccccc56)c4)cc3)cc2)cc1 ; c1ccc(-c2ccc(-c3ccc(N(c4cccc(-c5ccc6c(c5)c(-c5ccccc5)c(-c5ccccc5)c5ccccc56)c4)c4ccc(-c5ccccc5)c(-c5ccccc5)c4)cc3)cc2)cc1 ; c1ccc(-c2ccc(-c3ccc(N(c4ccc(-c5cccc(-c6ccccc6)c5-c5ccccc5)cc4)c4cccc(-c5ccc6c(c5)c(-c5ccccc5)c(-c5ccccc5)c5ccccc56)c4)cc3)cc2)cc1]

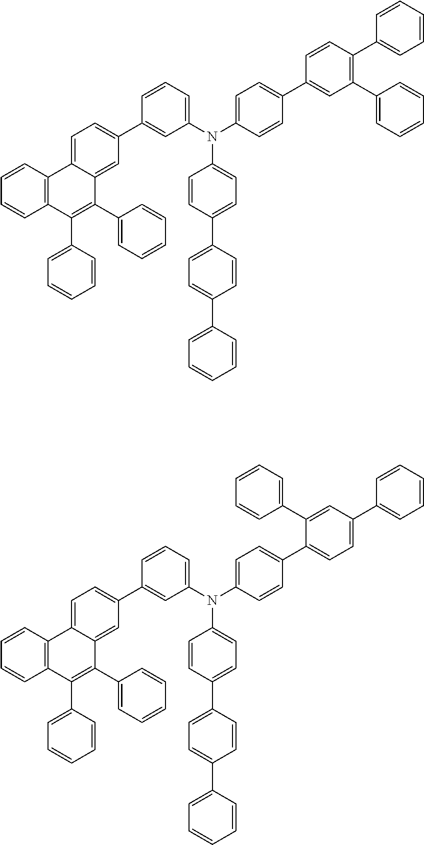 [SMILES: c1ccc(-c2ccc(-c3ccc(N(c4ccc(-c5ccc(-c6ccccc6)c(-c6ccccc6)c5)cc4)c4cccc(-c5ccc6c(c5)c(-c5ccccc5)c(-c5ccccc5)c5ccccc56)c4)cc3)cc2)cc1 ; c1ccc(-c2ccc(-c3ccc(N(c4ccc(-c5ccc(-c6ccccc6)cc5-c5ccccc5)cc4)c4cccc(-c5ccc6c(c5)c(-c5ccccc5)c(-c5ccccc5)c5ccccc56)c4)cc3)cc2)cc1]

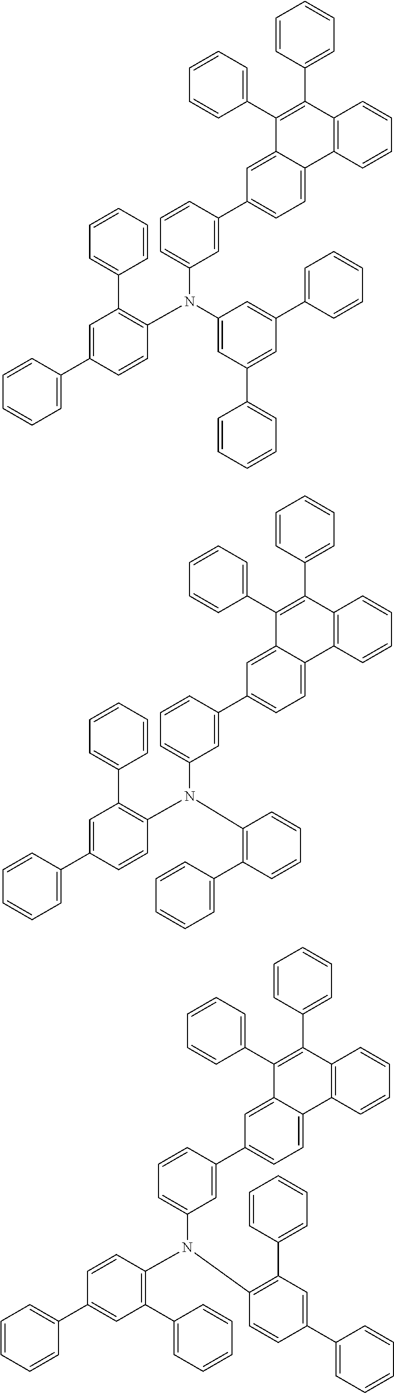 [SMILES: c1ccc(-c2cc(-c3ccccc3)cc(N(c3cccc(-c4ccc5c(c4)c(-c4ccccc4)c(-c4ccccc4)c4ccccc45)c3)c3ccc(-c4ccccc4)cc3-c3ccccc3)c2)cc1 ; c1ccc(-c2ccc(N(c3cccc(-c4ccc5c(c4)c(-c4ccccc4)c(-c4ccccc4)c4ccccc45)c3)c3ccccc3-c3ccccc3)c(-c3ccccc3)c2)cc1 ; c1ccc(-c2ccc(N(c3cccc(-c4ccc5c(c4)c(-c4ccccc4)c(-c4ccccc4)c4ccccc45)c3)c3ccc(-c4ccccc4)cc3-c3ccccc3)c(-c3ccccc3)c2)cc1]

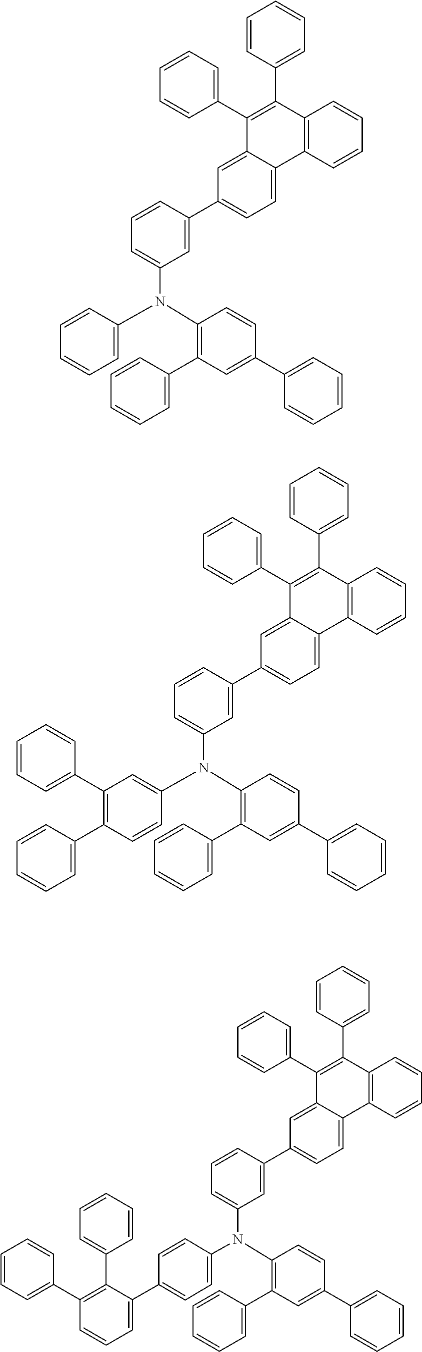

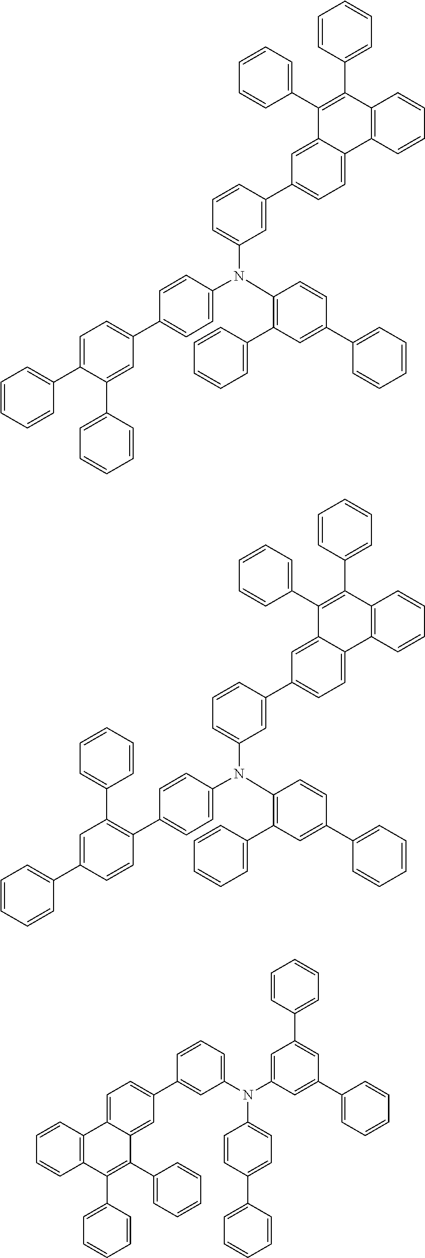 [SMILES: c1ccc(-c2ccc(N(c3ccc(-c4ccc(-c5ccccc5)c(-c5ccccc5)c4)cc3)c3cccc(-c4ccc5c(c4)c(-c4ccccc4)c(-c4ccccc4)c4ccccc45)c3)c(-c3ccccc3)c2)cc1 ; c1ccc(-c2ccc(-c3ccc(N(c4cccc(-c5ccc6c(c5)c(-c5ccccc5)c(-c5ccccc5)c5ccccc56)c4)c4ccc(-c5ccccc5)cc4-c4ccccc4)cc3)c(-c3ccccc3)c2)cc1 ; c1ccc(-c2ccc(N(c3cccc(-c4ccc5c(c4)c(-c4ccccc4)c(-c4ccccc4)c4ccccc45)c3)c3cc(-c4ccccc4)cc(-c4ccccc4)c3)cc2)cc1]

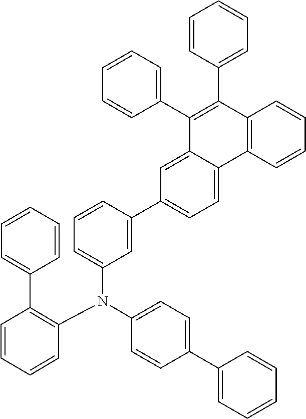

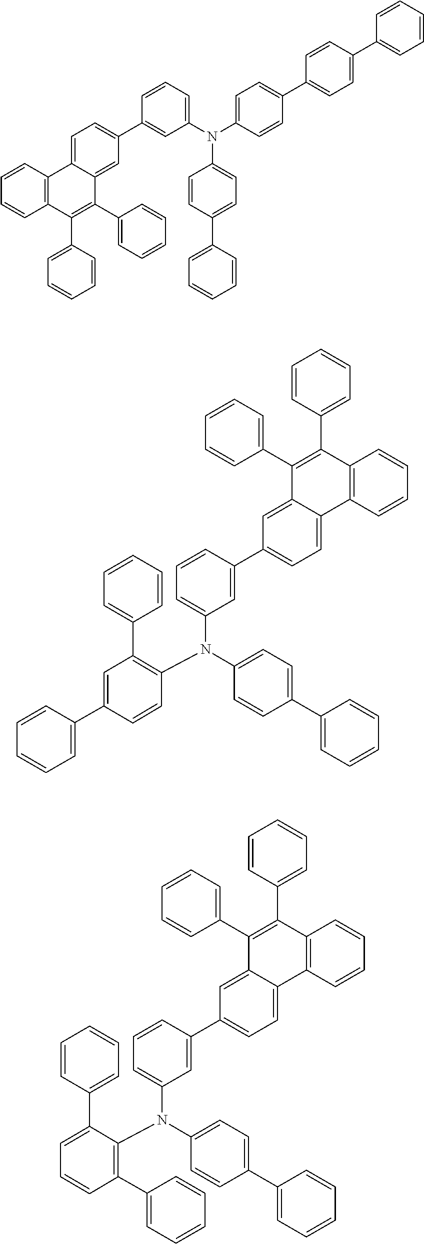

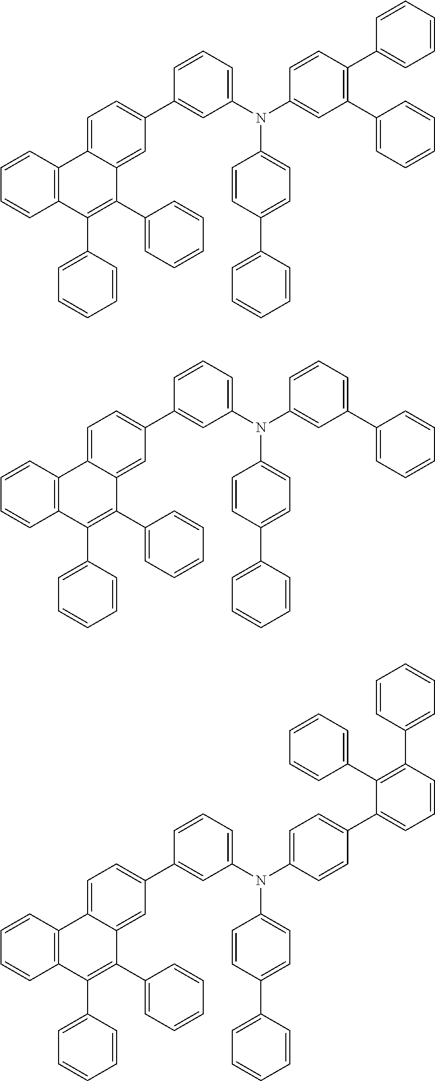 [SMILES: c1ccc(-c2ccc(N(c3cccc(-c4ccc5c(c4)c(-c4ccccc4)c(-c4ccccc4)c4ccccc45)c3)c3ccc(-c4ccccc4)c(-c4ccccc4)c3)cc2)cc1 ; c1ccc(-c2ccc(N(c3cccc(-c4ccccc4)c3)c3cccc(-c4ccc5c(c4)c(-c4ccccc4)c(-c4ccccc4)c4ccccc45)c3)cc2)cc1 ; c1ccc(-c2ccc(N(c3ccc(-c4cccc(-c5ccccc5)c4-c4ccccc4)cc3)c3cccc(-c4ccc5c(c4)c(-c4ccccc4)c(-c4ccccc4)c4ccccc45)c3)cc2)cc1]

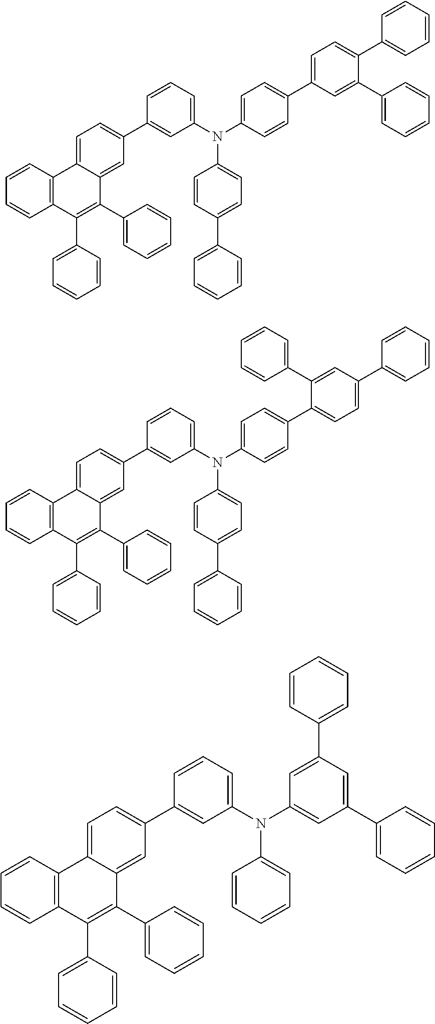 [SMILES: c1ccc(-c2ccc(N(c3ccc(-c4ccc(-c5ccccc5)c(-c5ccccc5)c4)cc3)c3cccc(-c4ccc5c(c4)c(-c4ccccc4)c(-c4ccccc4)c4ccccc45)c3)cc2)cc1 ; c1ccc(-c2ccc(N(c3ccc(-c4ccc(-c5ccccc5)cc4-c4ccccc4)cc3)c3cccc(-c4ccc5c(c4)c(-c4ccccc4)c(-c4ccccc4)c4ccccc45)c3)cc2)cc1 ; c1ccc(-c2cc(-c3ccccc3)cc(N(c3ccccc3)c3cccc(-c4ccc5c(c4)c(-c4ccccc4)c(-c4ccccc4)c4ccccc45)c3)c2)cc1]

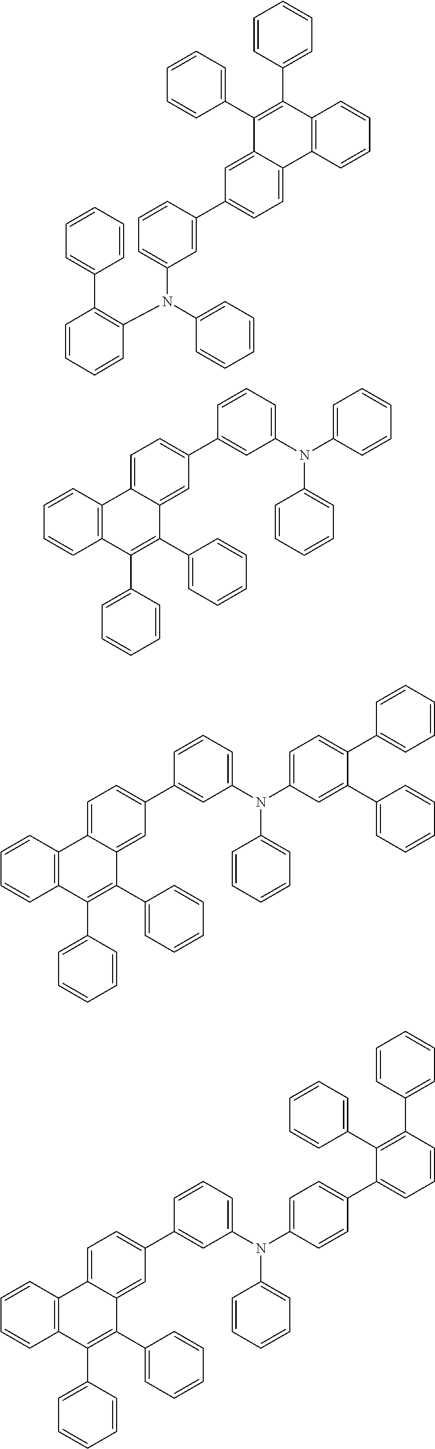

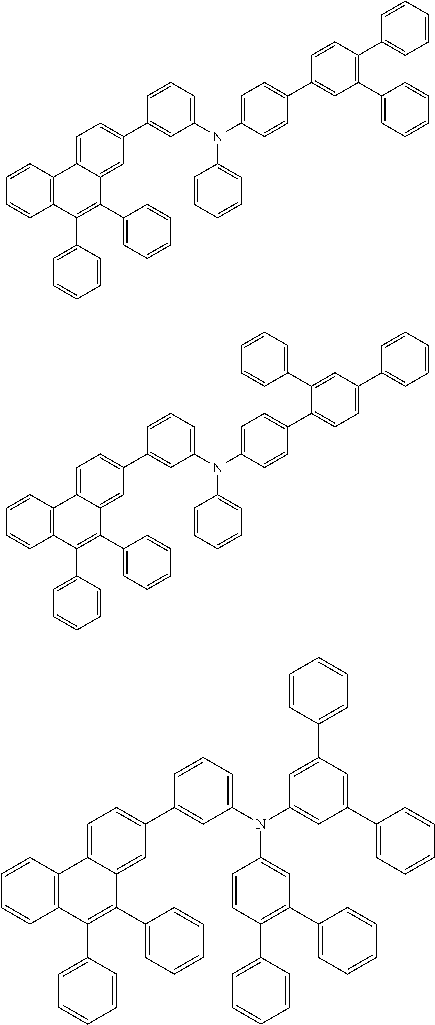 [SMILES: c1ccc(-c2ccc(-c3ccc(N(c4ccccc4)c4cccc(-c5ccc6c(c5)c(-c5ccccc5)c(-c5ccccc5)c5ccccc56)c4)cc3)cc2-c2ccccc2)cc1 ; c1ccc(-c2ccc(-c3ccc(N(c4ccccc4)c4cccc(-c5ccc6c(c5)c(-c5ccccc5)c(-c5ccccc5)c5ccccc56)c4)cc3)c(-c3ccccc3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3ccc(-c4ccccc4)c(-c4ccccc4)c3)c2)cc1]

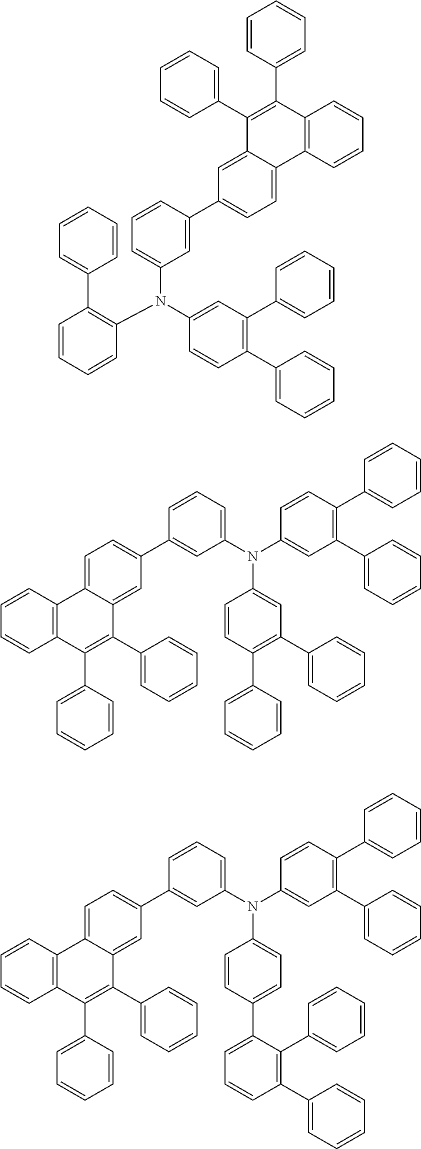

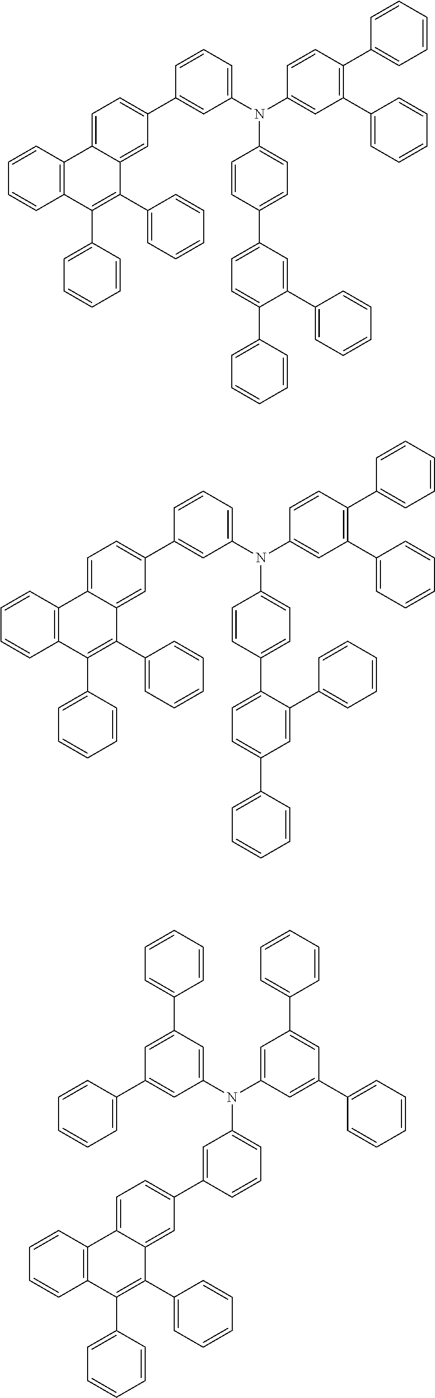 [SMILES: c1ccc(-c2ccc(-c3ccc(N(c4cccc(-c5ccc6c(c5)c(-c5ccccc5)c(-c5ccccc5)c5ccccc56)c4)c4ccc(-c5ccccc5)c(-c5ccccc5)c4)cc3)cc2-c2ccccc2)cc1 ; c1ccc(-c2ccc(-c3ccc(N(c4cccc(-c5ccc6c(c5)c(-c5ccccc5)c(-c5ccccc5)c5ccccc56)c4)c4ccc(-c5ccccc5)c(-c5ccccc5)c4)cc3)c(-c3ccccc3)c2)cc1 ; c1ccc(-c2cc(-c3ccccc3)cc(N(c3cccc(-c4ccc5c(c4)c(-c4ccccc4)c(-c4ccccc4)c4ccccc45)c3)c3cc(-c4ccccc4)cc(-c4ccccc4)c3)c2)cc1]

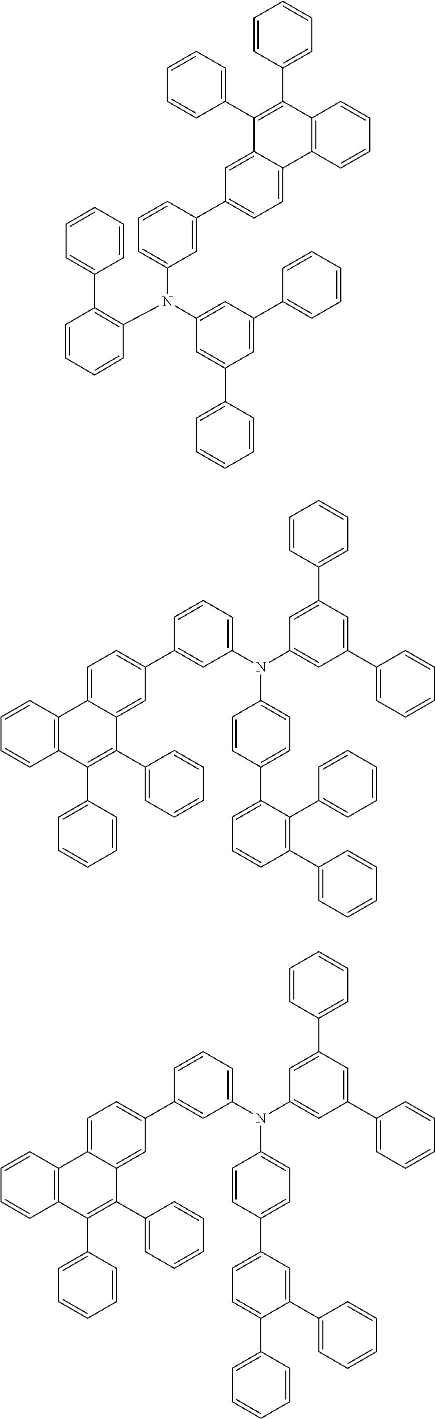

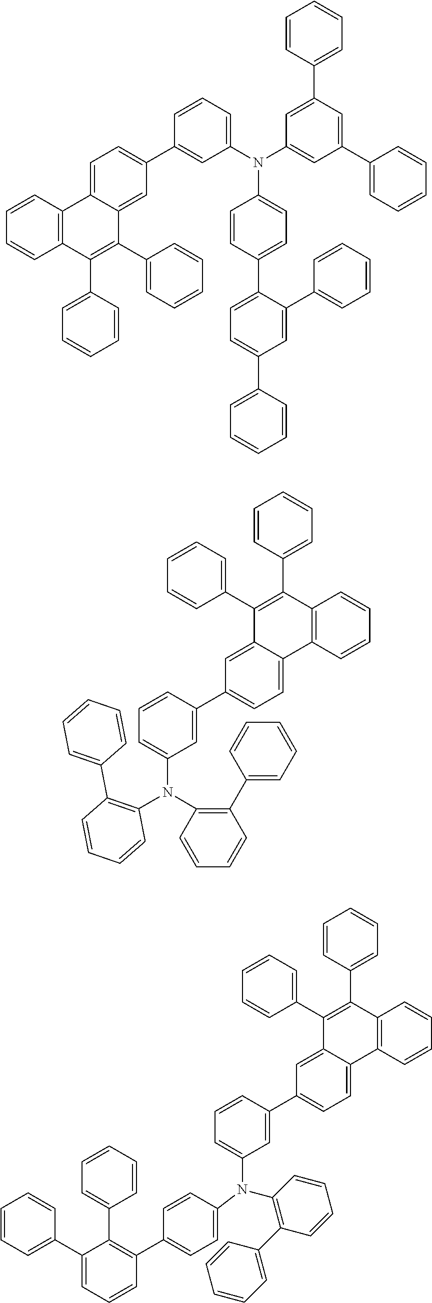 [SMILES: c1ccc(-c2cc(-c3ccccc3)cc(N(c3ccc(-c4ccc(-c5ccccc5)cc4-c4ccccc4)cc3)c3cccc(-c4ccc5c(c4)c(-c4ccccc4)c(-c4ccccc4)c4ccccc45)c3)c2)cc1 ; c1ccc(-c2ccccc2N(c2cccc(-c3ccc4c(c3)c(-c3ccccc3)c(-c3ccccc3)c3ccccc34)c2)c2ccccc2-c2ccccc2)cc1 ; c1ccc(-c2ccccc2N(c2ccc(-c3cccc(-c4ccccc4)c3-c3ccccc3)cc2)c2cccc(-c3ccc4c(c3)c(-c3ccccc3)c(-c3ccccc3)c3ccccc34)c2)cc1]

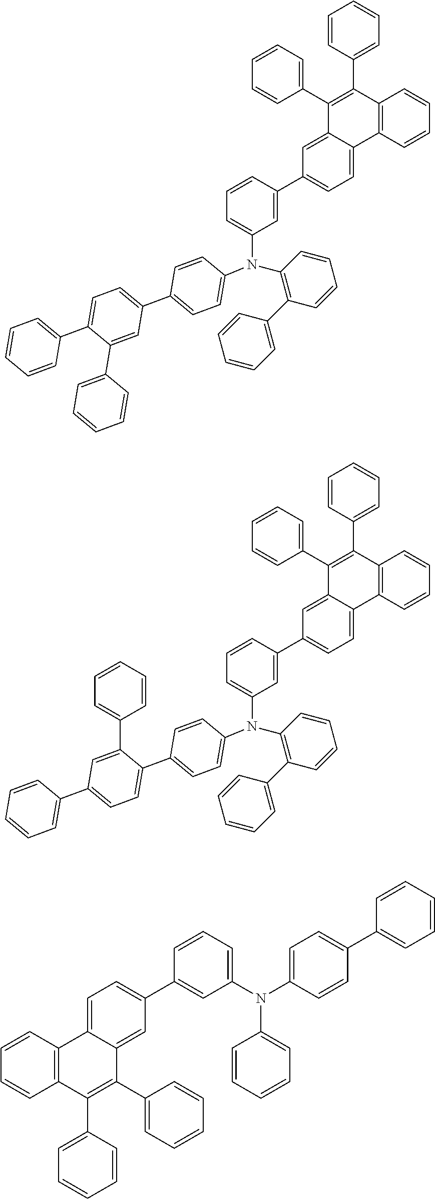 [SMILES: c1ccc(-c2ccc(-c3ccc(N(c4cccc(-c5ccc6c(c5)c(-c5ccccc5)c(-c5ccccc5)c5ccccc56)c4)c4ccccc4-c4ccccc4)cc3)cc2-c2ccccc2)cc1 ; c1ccc(-c2ccc(-c3ccc(N(c4cccc(-c5ccc6c(c5)c(-c5ccccc5)c(-c5ccccc5)c5ccccc56)c4)c4ccccc4-c4ccccc4)cc3)c(-c3ccccc3)c2)cc1 ; c1ccc(-c2ccc(N(c3ccccc3)c3cccc(-c4ccc5c(c4)c(-c4ccccc4)c(-c4ccccc4)c4ccccc45)c3)cc2)cc1]

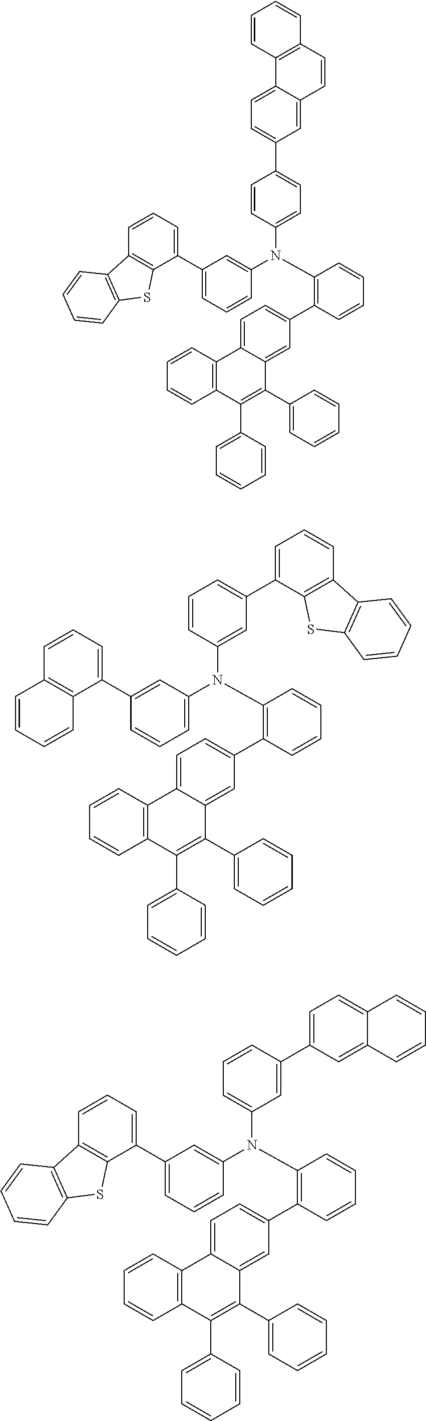 [SMILES: c1ccc(-c2c(-c3ccccc3)c3cc(-c4ccccc4N(c4ccc(-c5ccc6c(ccc7ccccc76)c5)cc4)c4cccc(-c5cccc6c5sc5ccccc56)c4)ccc3c3ccccc23)cc1 ; c1ccc(-c2c(-c3ccccc3)c3cc(-c4ccccc4N(c4cccc(-c5cccc6ccccc56)c4)c4cccc(-c5cccc6c5sc5ccccc56)c4)ccc3c3ccccc23)cc1 ; c1ccc(-c2c(-c3ccccc3)c3cc(-c4ccccc4N(c4cccc(-c5ccc6ccccc6c5)c4)c4cccc(-c5cccc6c5sc5ccccc56)c4)ccc3c3ccccc23)cc1]

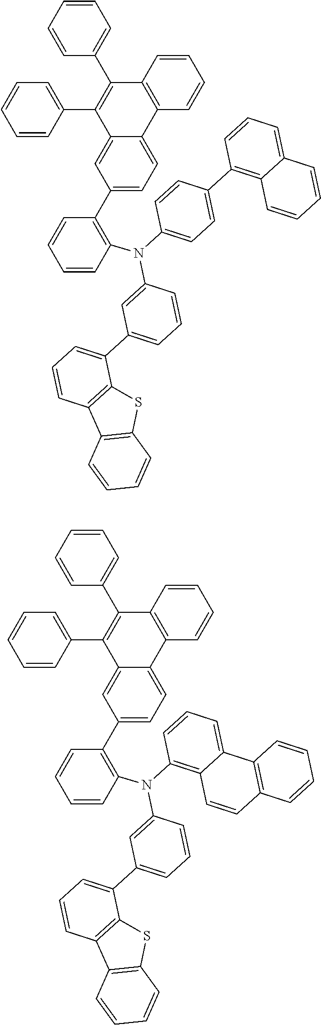

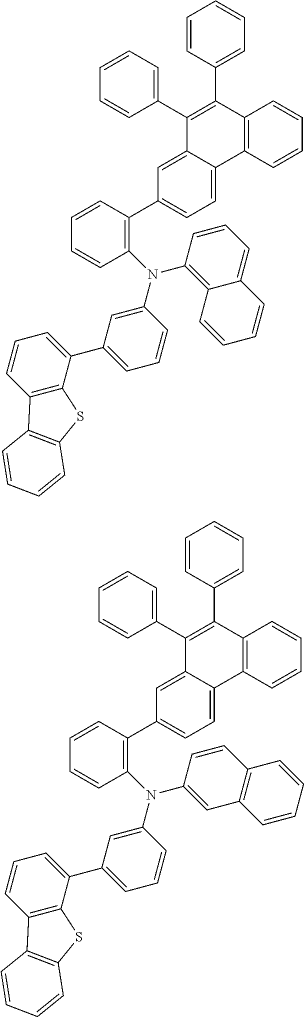 [SMILES: c1ccc(-c2c(-c3ccccc3)c3cc(-c4ccccc4N(c4cccc(-c5cccc6c5sc5ccccc56)c4)c4cccc5ccccc45)ccc3c3ccccc23)cc1 ; c1ccc(-c2c(-c3ccccc3)c3cc(-c4ccccc4N(c4cccc(-c5cccc6c5sc5ccccc56)c4)c4ccc5ccccc5c4)ccc3c3ccccc23)cc1]

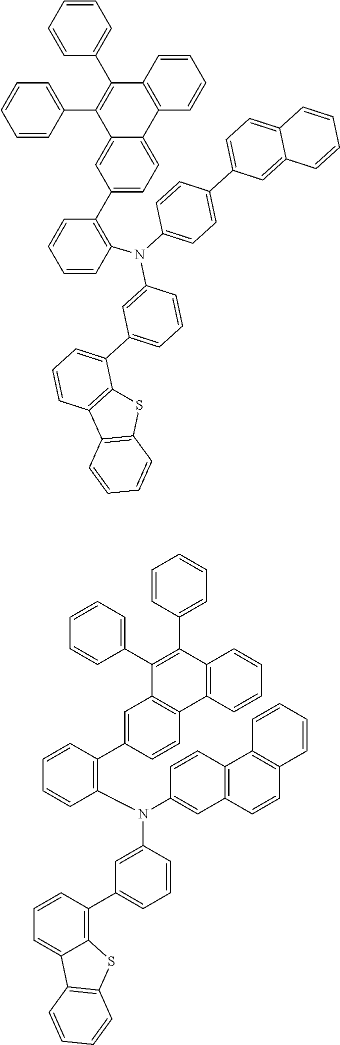 [SMILES: c1ccc(-c2c(-c3ccccc3)c3cc(-c4ccccc4N(c4ccc(-c5ccc6ccccc6c5)cc4)c4cccc(-c5cccc6c5sc5ccccc56)c4)ccc3c3ccccc23)cc1 ; c1ccc(-c2c(-c3ccccc3)c3cc(-c4ccccc4N(c4cccc(-c5cccc6c5sc5ccccc56)c4)c4ccc5c(ccc6ccccc65)c4)ccc3c3ccccc23)cc1]

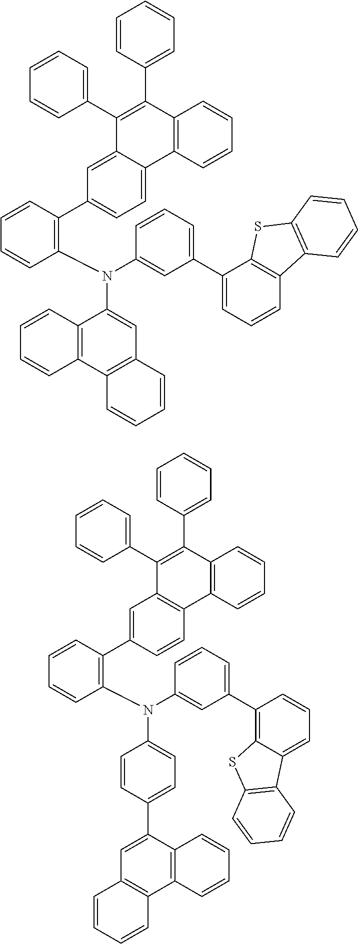 [SMILES: c1ccc(-c2c(-c3ccccc3)c3cc(-c4ccccc4N(c4cccc(-c5cccc6c5sc5ccccc56)c4)c4cc5ccccc5c5ccccc45)ccc3c3ccccc23)cc1 ; c1ccc(-c2c(-c3ccccc3)c3cc(-c4ccccc4N(c4ccc(-c5cc6ccccc6c6ccccc56)cc4)c4cccc(-c5cccc6c5sc5ccccc56)c4)ccc3c3ccccc23)cc1]

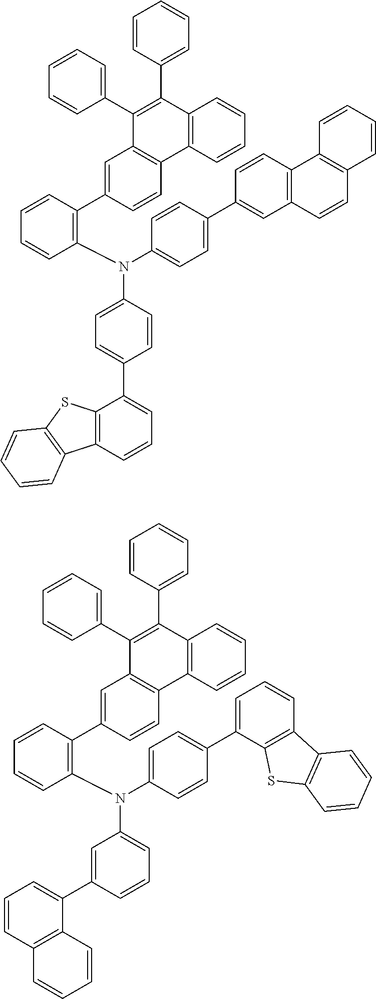 [SMILES: c1ccc(-c2c(-c3ccccc3)c3cc(-c4ccccc4N(c4ccc(-c5ccc6c(ccc7ccccc76)c5)cc4)c4ccc(-c5cccc6c5sc5ccccc56)cc4)ccc3c3ccccc23)cc1 ; c1ccc(-c2c(-c3ccccc3)c3cc(-c4ccccc4N(c4ccc(-c5cccc6c5sc5ccccc56)cc4)c4cccc(-c5cccc6ccccc56)c4)ccc3c3ccccc23)cc1]

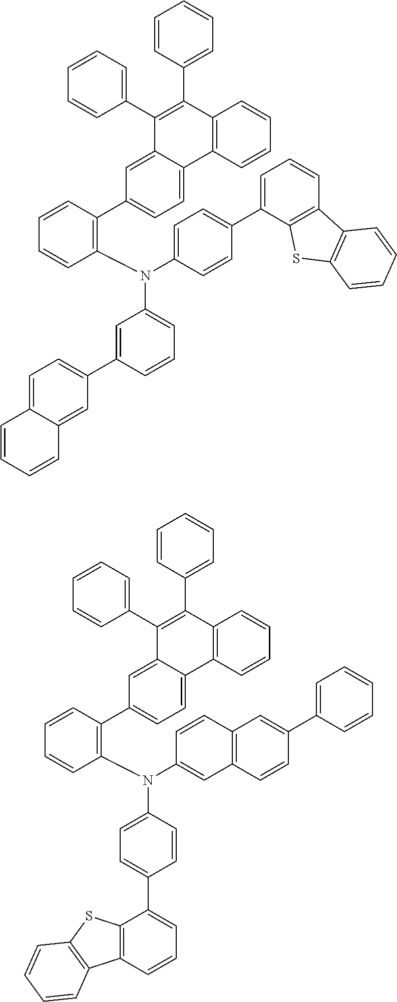 [SMILES: c1ccc(-c2c(-c3ccccc3)c3cc(-c4ccccc4N(c4ccc(-c5cccc6c5sc5ccccc56)cc4)c4cccc(-c5ccc6ccccc6c5)c4)ccc3c3ccccc23)cc1 ; c1ccc(-c2ccc3cc(N(c4ccc(-c5cccc6c5sc5ccccc56)cc4)c4ccccc4-c4ccc5c(c4)c(-c4ccccc4)c(-c4ccccc4)c4ccccc45)ccc3c2)cc1]

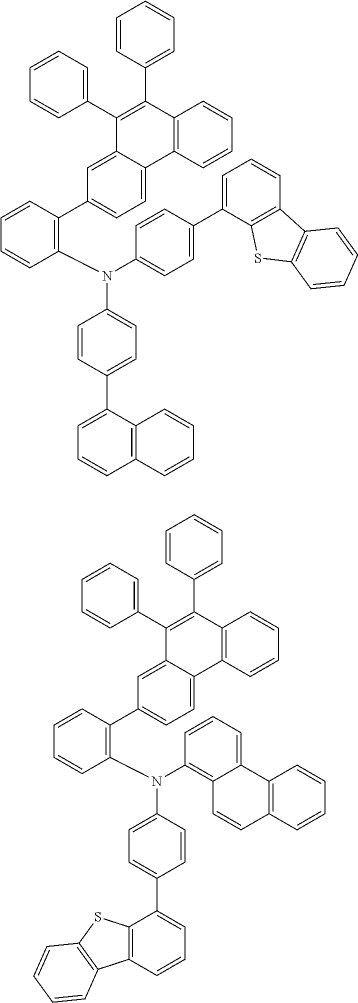 [SMILES: c1ccc(-c2c(-c3ccccc3)c3cc(-c4ccccc4N(c4ccc(-c5cccc6ccccc56)cc4)c4ccc(-c5cccc6c5sc5ccccc56)cc4)ccc3c3ccccc23)cc1 ; c1ccc(-c2c(-c3ccccc3)c3cc(-c4ccccc4N(c4ccc(-c5cccc6c5sc5ccccc56)cc4)c4cccc5c4ccc4ccccc45)ccc3c3ccccc23)cc1]

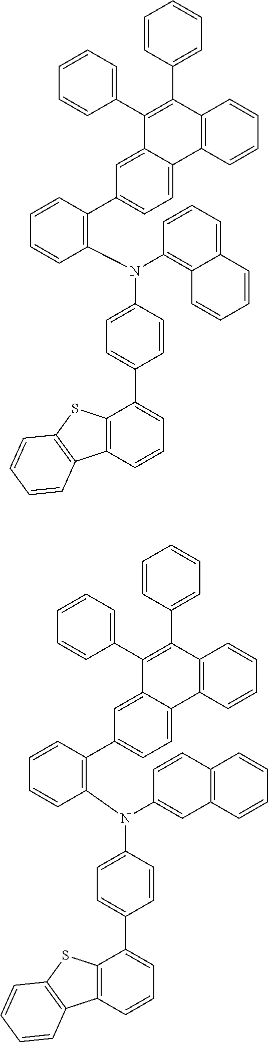 [SMILES: c1ccc(-c2c(-c3ccccc3)c3cc(-c4ccccc4N(c4ccc(-c5cccc6c5sc5ccccc56)cc4)c4cccc5ccccc45)ccc3c3ccccc23)cc1 ; c1ccc(-c2c(-c3ccccc3)c3cc(-c4ccccc4N(c4ccc(-c5cccc6c5sc5ccccc56)cc4)c4ccc5ccccc5c4)ccc3c3ccccc23)cc1]

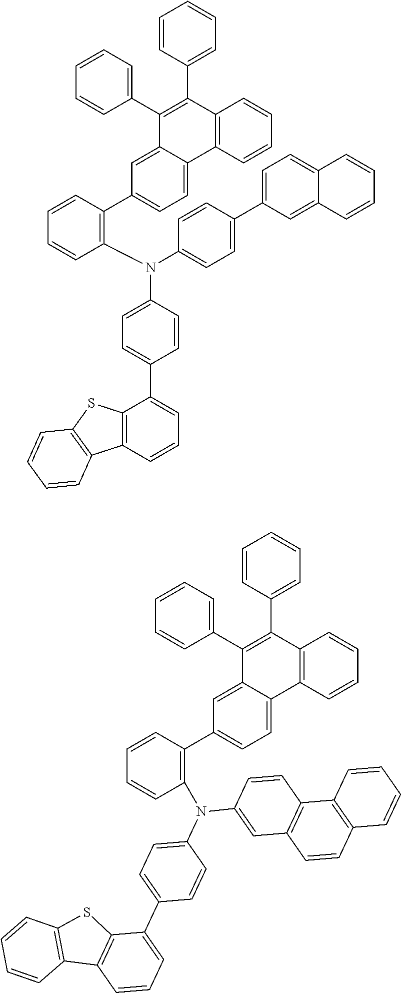 [SMILES: c1ccc(-c2c(-c3ccccc3)c3cc(-c4ccccc4N(c4ccc(-c5ccc6ccccc6c5)cc4)c4ccc(-c5cccc6c5sc5ccccc56)cc4)ccc3c3ccccc23)cc1 ; c1ccc(-c2c(-c3ccccc3)c3cc(-c4ccccc4N(c4ccc(-c5cccc6c5sc5ccccc56)cc4)c4ccc5c(ccc6ccccc65)c4)ccc3c3ccccc23)cc1]

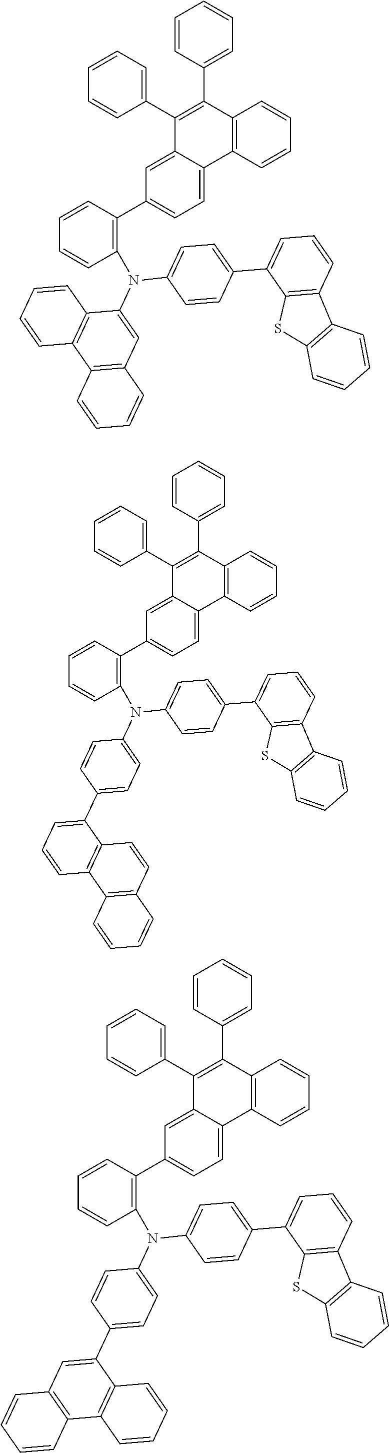 [SMILES: c1ccc(-c2c(-c3ccccc3)c3cc(-c4ccccc4N(c4ccc(-c5cccc6c5sc5ccccc56)cc4)c4cc5ccccc5c5ccccc45)ccc3c3ccccc23)cc1 ; c1ccc(-c2c(-c3ccccc3)c3cc(-c4ccccc4N(c4ccc(-c5cccc6c5ccc5ccccc56)cc4)c4ccc(-c5cccc6c5sc5ccccc56)cc4)ccc3c3ccccc23)cc1 ; c1ccc(-c2c(-c3ccccc3)c3cc(-c4ccccc4N(c4ccc(-c5cc6ccccc6c6ccccc56)cc4)c4ccc(-c5cccc6c5sc5ccccc56)cc4)ccc3c3ccccc23)cc1]

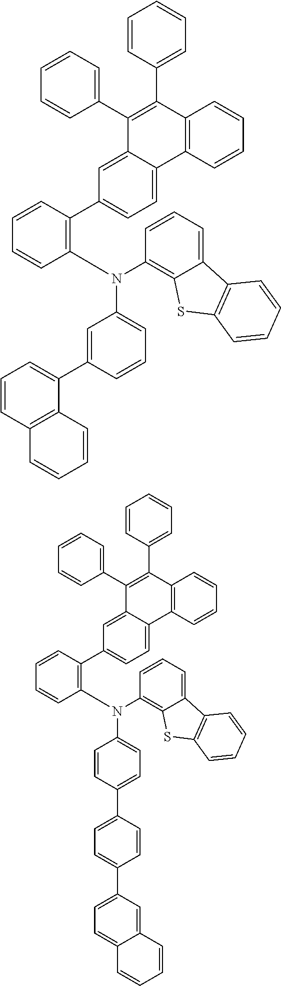 [SMILES: c1ccc(-c2c(-c3ccccc3)c3cc(-c4ccccc4N(c4cccc(-c5cccc6ccccc56)c4)c4cccc5c4sc4ccccc45)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc5c4sc4ccccc45)ccc3c3ccccc23)cc1]

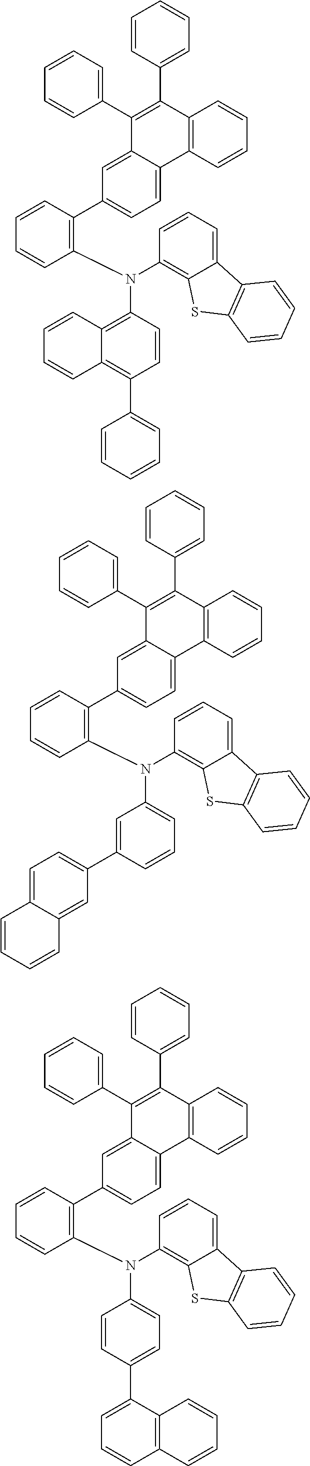

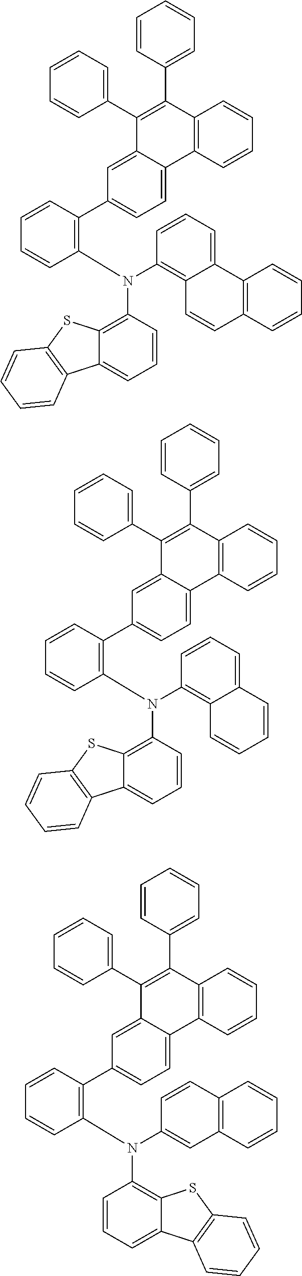 [SMILES: c1ccc(-c2c(-c3ccccc3)c3cc(-c4ccccc4N(c4cccc5c4ccc4ccccc45)c4cccc5c4sc4ccccc45)ccc3c3ccccc23)cc1 ; c1ccc(-c2c(-c3ccccc3)c3cc(-c4ccccc4N(c4cccc5ccccc45)c4cccc5c4sc4ccccc45)ccc3c3ccccc23)cc1 ; c1ccc(-c2c(-c3ccccc3)c3cc(-c4ccccc4N(c4ccc5ccccc5c4)c4cccc5c4sc4ccccc45)ccc3c3ccccc23)cc1]

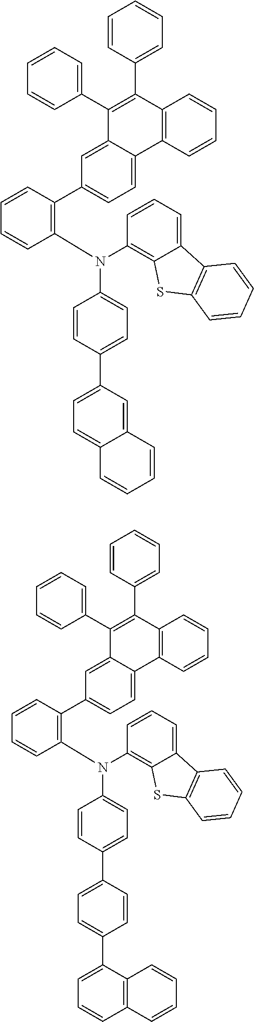

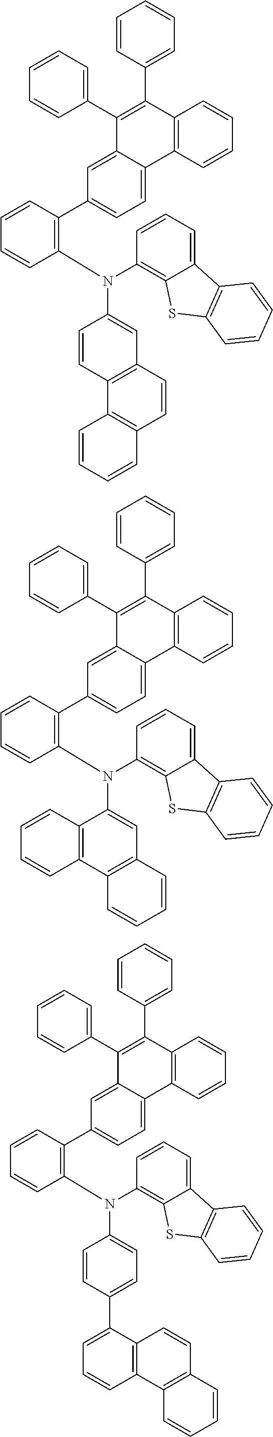

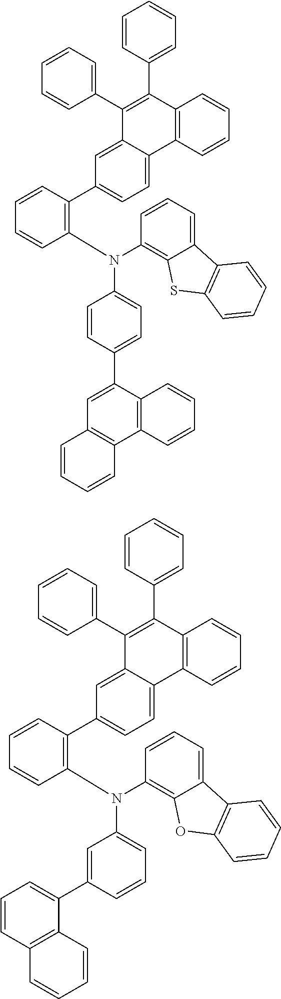 [SMILES: c1ccc(-c2c(-c3ccccc3)c3cc(-c4ccccc4N(c4ccc(-c5cc6ccccc6c6ccccc56)cc4)c4cccc5c4sc4ccccc45)ccc3c3ccccc23)cc1 ; c1ccc(-c2c(-c3ccccc3)c3cc(-c4ccccc4N(c4cccc(-c5cccc6ccccc56)c4)c4cccc5c4oc4ccccc45)ccc3c3ccccc23)cc1]

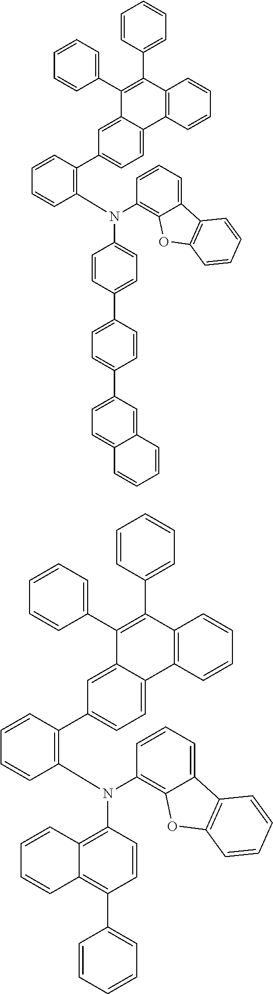

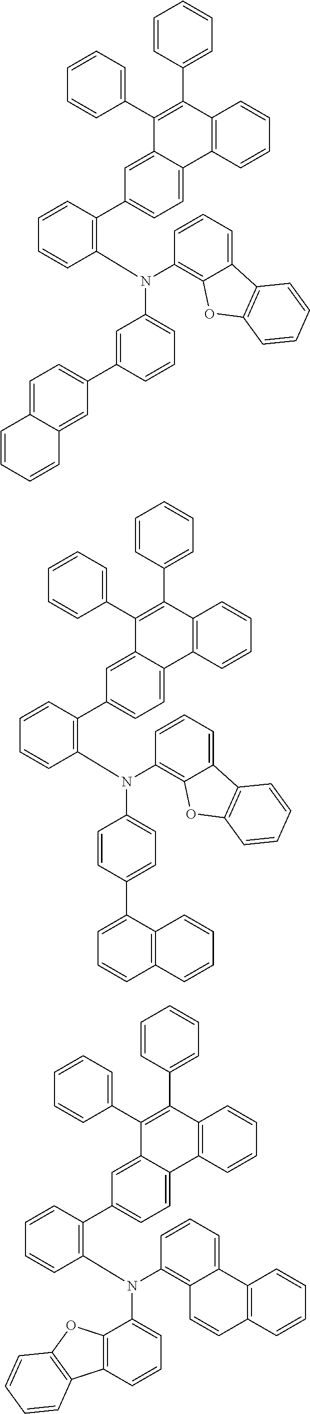

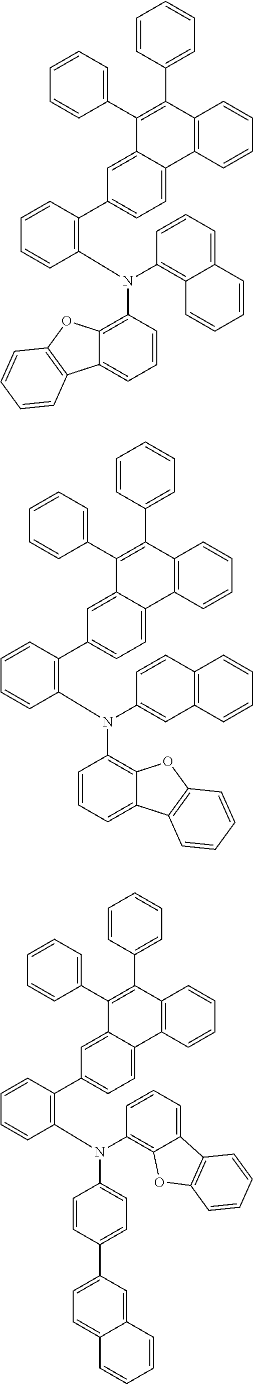 [SMILES: c1ccc(-c2c(-c3ccccc3)c3cc(-c4ccccc4N(c4cccc5ccccc45)c4cccc5c4oc4ccccc45)ccc3c3ccccc23)cc1 ; c1ccc(-c2c(-c3ccccc3)c3cc(-c4ccccc4N(c4ccc5ccccc5c4)c4cccc5c4oc4ccccc45)ccc3c3ccccc23)cc1 ; c1ccc(-c2c(-c3ccccc3)c3cc(-c4ccccc4N(c4ccc(-c5ccc6ccccc6c5)cc4)c4cccc5c4oc4ccccc45)ccc3c3ccccc23)cc1]

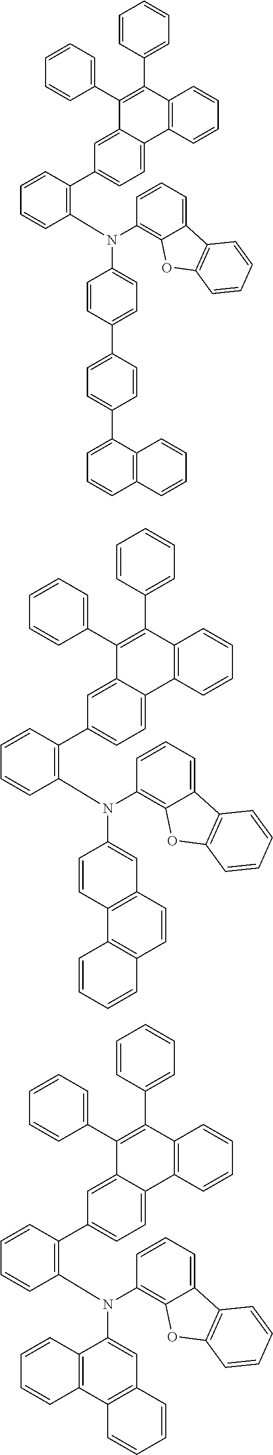

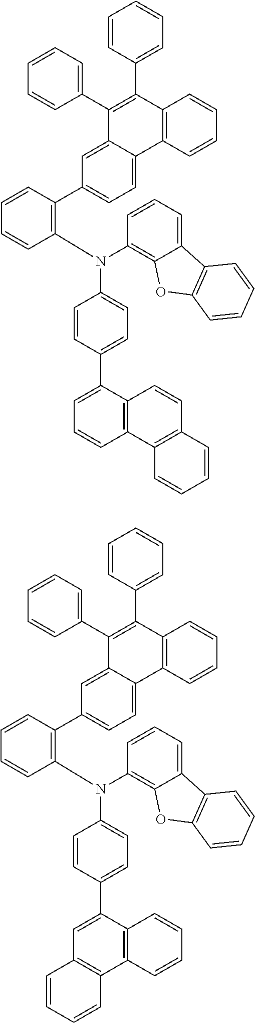 [SMILES: c1ccc(-c2c(-c3ccccc3)c3cc(-c4ccccc4N(c4ccc(-c5cccc6c5ccc5ccccc56)cc4)c4cccc5c4oc4ccccc45)ccc3c3ccccc23)cc1 ; c1ccc(-c2c(-c3ccccc3)c3cc(-c4ccccc4N(c4ccc(-c5cc6ccccc6c6ccccc56)cc4)c4cccc5c4oc4ccccc45)ccc3c3ccccc23)cc1]

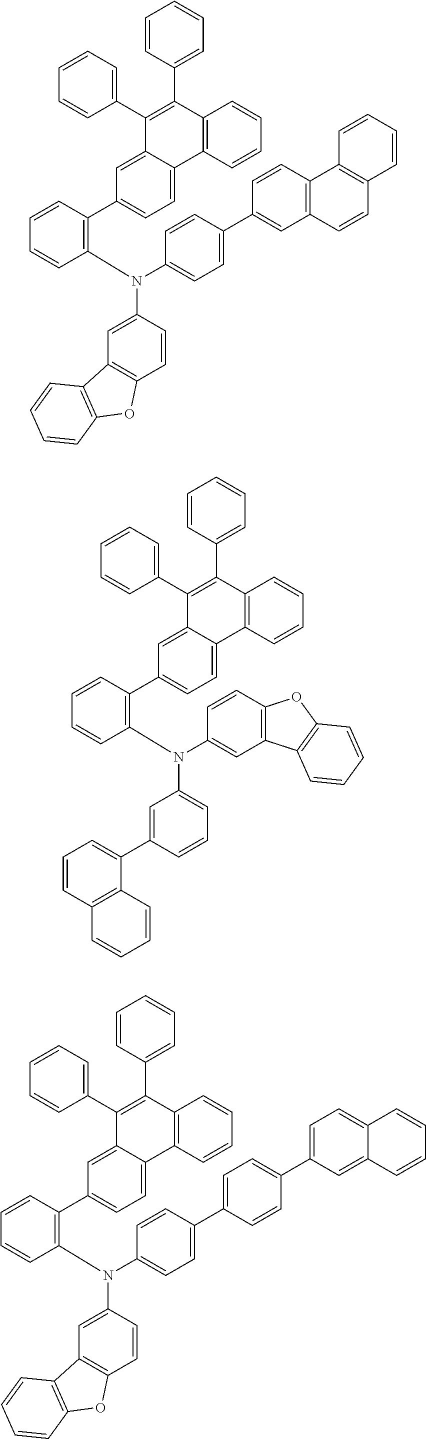

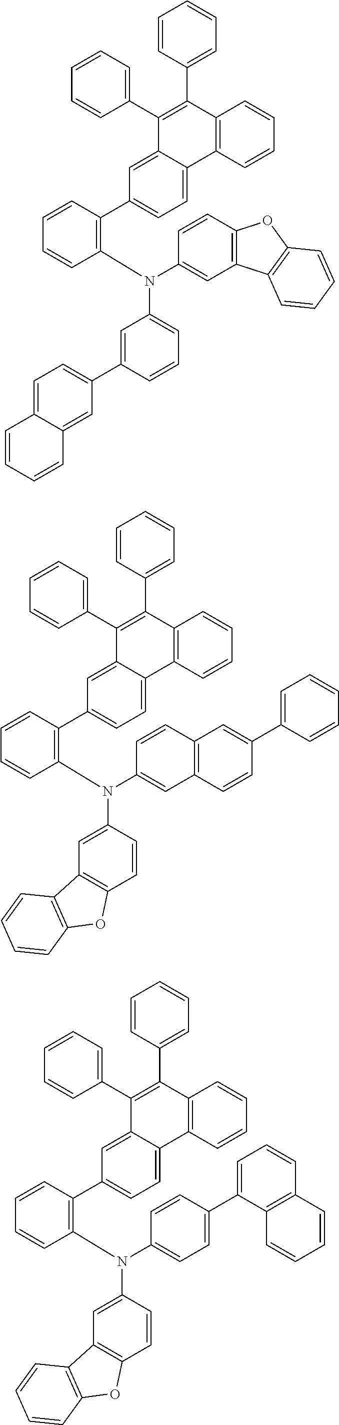 [SMILES: c1ccc(-c2c(-c3ccccc3)c3cc(-c4ccccc4N(c4cccc(-c5ccc6ccccc6c5)c4)c4ccc5oc6ccccc6c5c4)ccc3c3ccccc23)cc1 ; c1ccc(-c2ccc3cc(N(c4ccc5oc6ccccc6c5c4)c4ccccc4-c4ccc5c(c4)c(-c4ccccc4)c(-c4ccccc4)c4ccccc45)ccc3c2)cc1 ; c1ccc(-c2c(-c3ccccc3)c3cc(-c4ccccc4N(c4ccc(-c5cccc6ccccc56)cc4)c4ccc5oc6ccccc6c5c4)ccc3c3ccccc23)cc1]

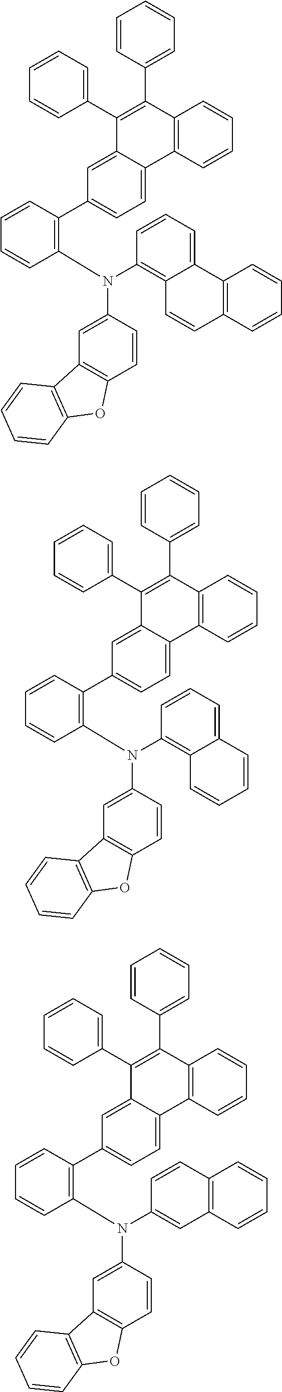 [SMILES: c1ccc(-c2c(-c3ccccc3)c3cc(-c4ccccc4N(c4ccc5oc6ccccc6c5c4)c4cccc5c4ccc4ccccc45)ccc3c3ccccc23)cc1 ; c1ccc(-c2c(-c3ccccc3)c3cc(-c4ccccc4N(c4ccc5oc6ccccc6c5c4)c4cccc5ccccc45)ccc3c3ccccc23)cc1 ; c1ccc(-c2c(-c3ccccc3)c3cc(-c4ccccc4N(c4ccc5ccccc5c4)c4ccc5oc6ccccc6c5c4)ccc3c3ccccc23)cc1]

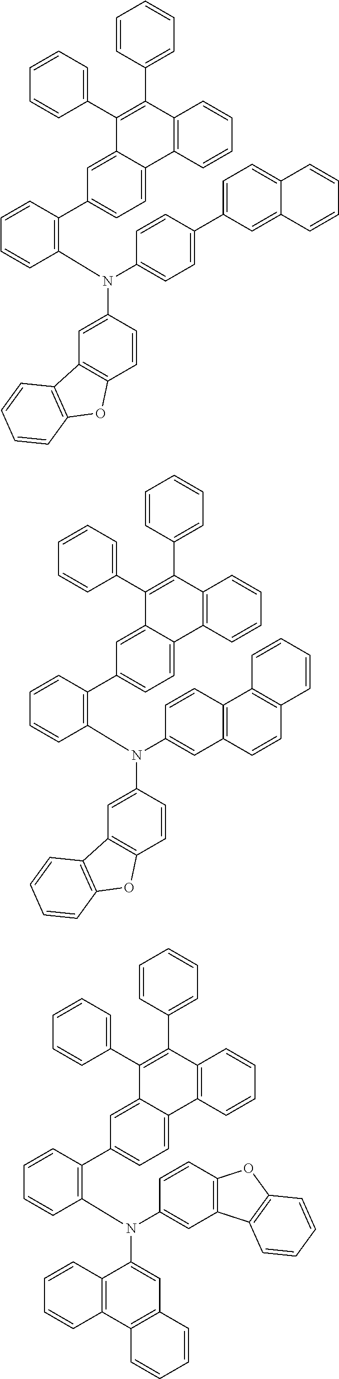 [SMILES: c1ccc(-c2c(-c3ccccc3)c3cc(-c4ccccc4N(c4ccc(-c5ccc6ccccc6c5)cc4)c4ccc5oc6ccccc6c5c4)ccc3c3ccccc23)cc1 ; c1ccc(-c2c(-c3ccccc3)c3cc(-c4ccccc4N(c4ccc5c(ccc6ccccc65)c4)c4ccc5oc6ccccc6c5c4)ccc3c3ccccc23)cc1 ; c1ccc(-c2c(-c3ccccc3)c3cc(-c4ccccc4N(c4ccc5oc6ccccc6c5c4)c4cc5ccccc5c5ccccc45)ccc3c3ccccc23)cc1]

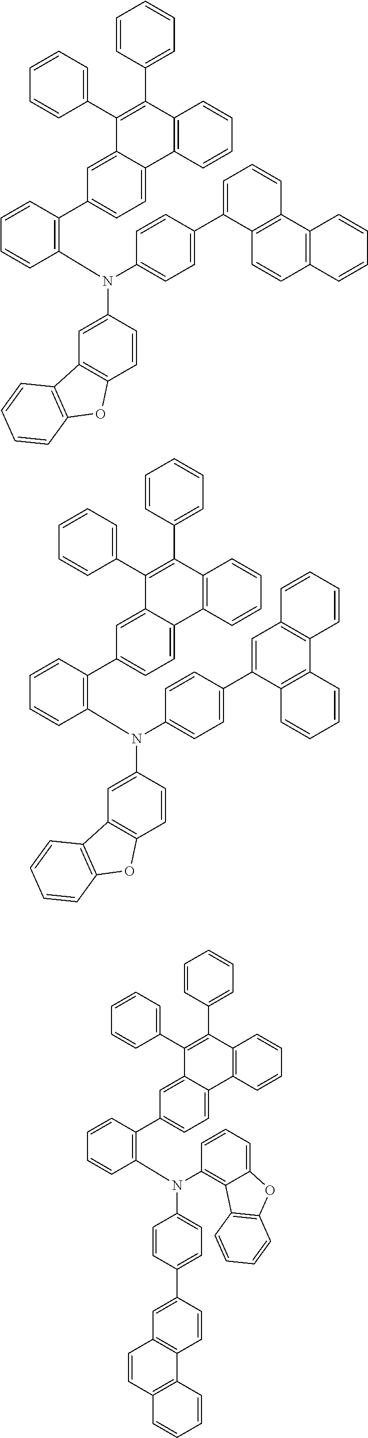 [SMILES: c1ccc(-c2c(-c3ccccc3)c3cc(-c4ccccc4N(c4ccc(-c5cccc6c5ccc5ccccc56)cc4)c4ccc5oc6ccccc6c5c4)ccc3c3ccccc23)cc1 ; c1ccc(-c2c(-c3ccccc3)c3cc(-c4ccccc4N(c4ccc(-c5cc6ccccc6c6ccccc56)cc4)c4ccc5oc6ccccc6c5c4)ccc3c3ccccc23)cc1 ; c1ccc(-c2c(-c3ccccc3)c3cc(-c4ccccc4N(c4ccc(-c5ccc6c(ccc7ccccc76)c5)cc4)c4cccc5oc6ccccc6c45)ccc3c3ccccc23)cc1]

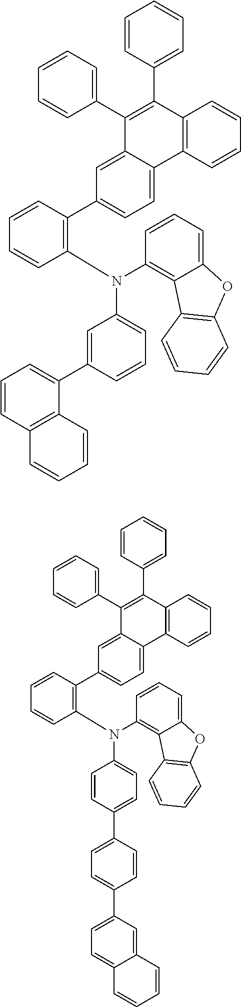 [SMILES: c1ccc(-c2c(-c3ccccc3)c3cc(-c4ccccc4N(c4cccc(-c5cccc6ccccc56)c4)c4cccc5oc6ccccc6c45)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc5oc6ccccc6c45)ccc3c3ccccc23)cc1]

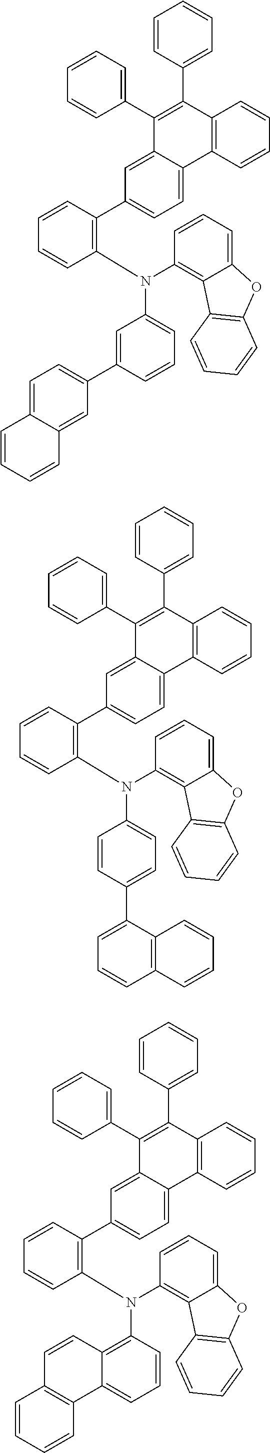

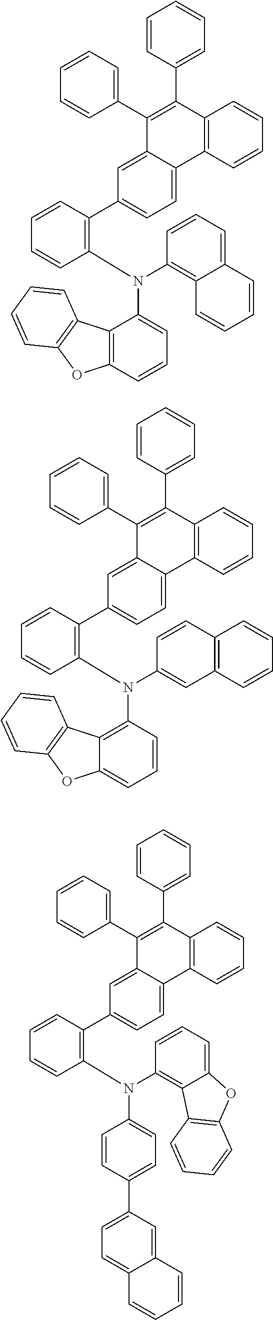

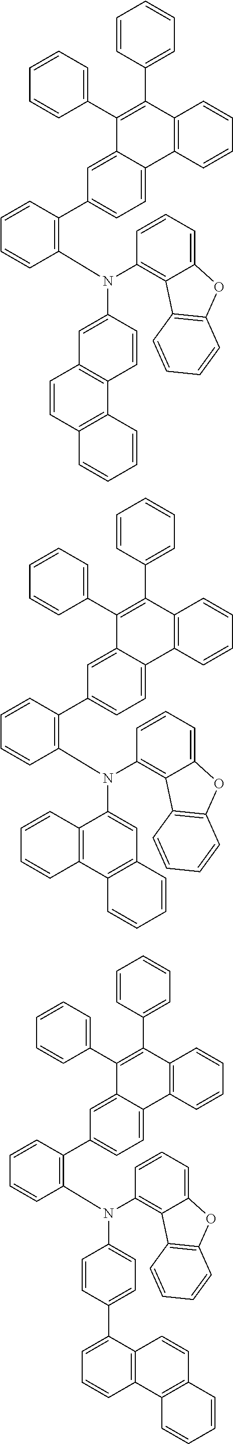

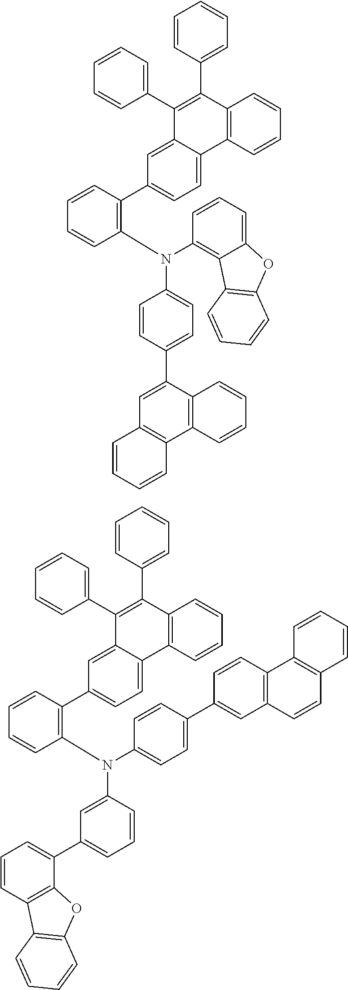

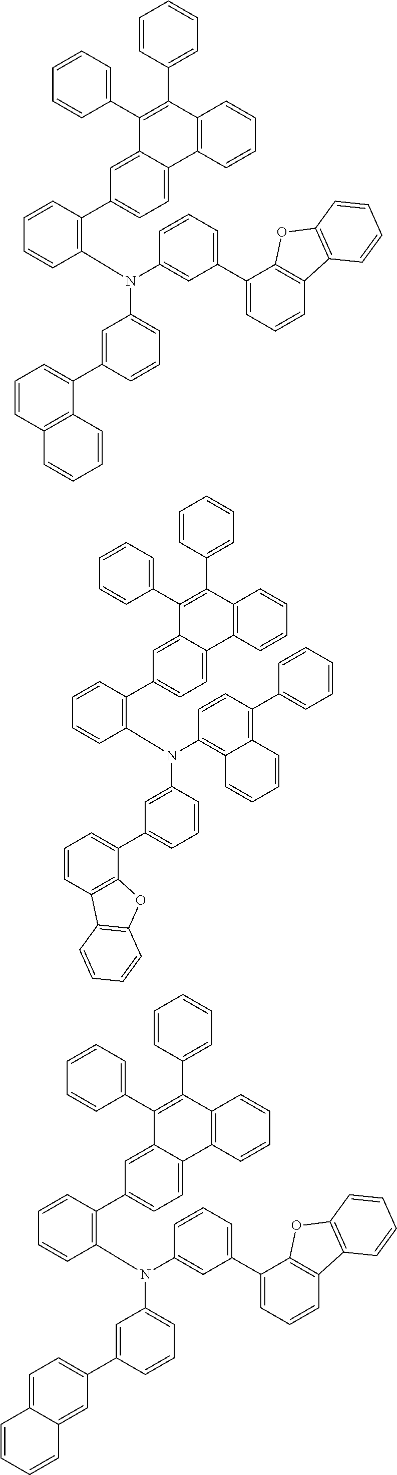

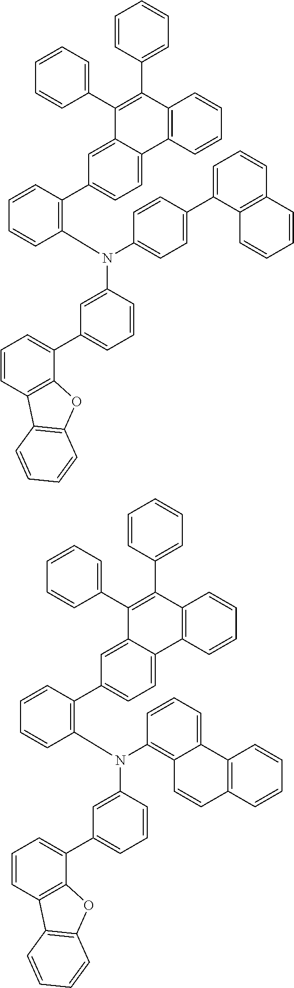 [SMILES: c1ccc(-c2c(-c3ccccc3)c3cc(-c4ccccc4N(c4ccc(-c5cccc6ccccc56)cc4)c4cccc(-c5cccc6c5oc5ccccc56)c4)ccc3c3ccccc23)cc1 ; c1ccc(-c2c(-c3ccccc3)c3cc(-c4ccccc4N(c4cccc(-c5cccc6c5oc5ccccc56)c4)c4cccc5c4ccc4ccccc45)ccc3c3ccccc23)cc1]

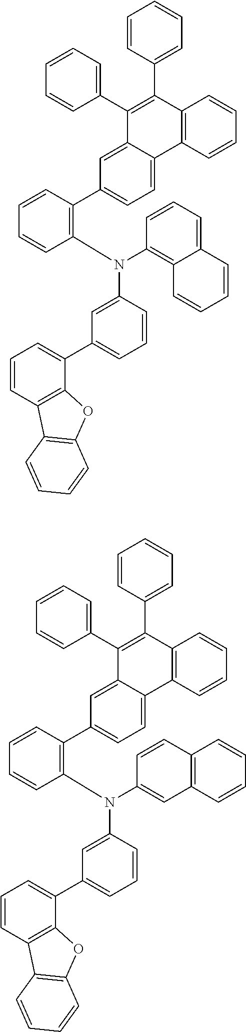 [SMILES: c1ccc(-c2c(-c3ccccc3)c3cc(-c4ccccc4N(c4cccc(-c5cccc6c5oc5ccccc56)c4)c4cccc5ccccc45)ccc3c3ccccc23)cc1 ; c1ccc(-c2c(-c3ccccc3)c3cc(-c4ccccc4N(c4cccc(-c5cccc6c5oc5ccccc56)c4)c4ccc5ccccc5c4)ccc3c3ccccc23)cc1]

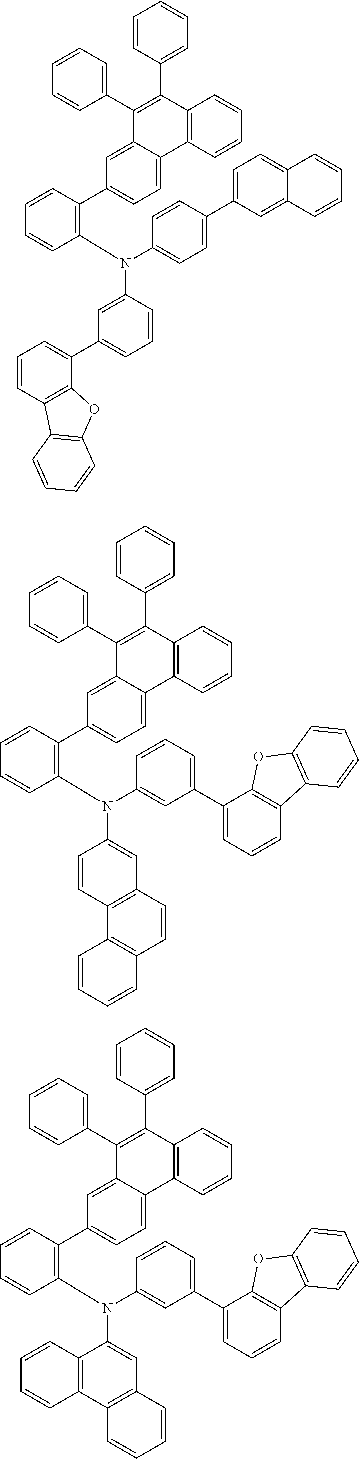

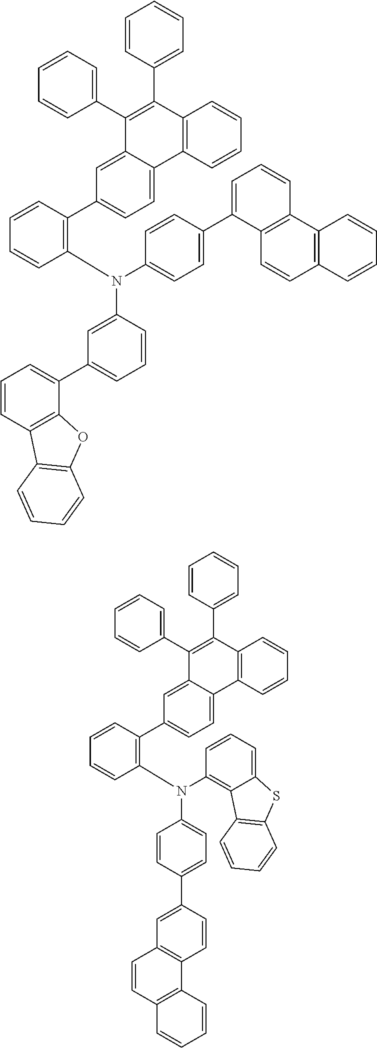 [SMILES: c1ccc(-c2c(-c3ccccc3)c3cc(-c4ccccc4N(c4ccc(-c5cccc6c5ccc5ccccc56)cc4)c4cccc(-c5cccc6c5oc5ccccc56)c4)ccc3c3ccccc23)cc1 ; c1ccc(-c2c(-c3ccccc3)c3cc(-c4ccccc4N(c4ccc(-c5ccc6c(ccc7ccccc76)c5)cc4)c4cccc5sc6ccccc6c45)ccc3c3ccccc23)cc1]

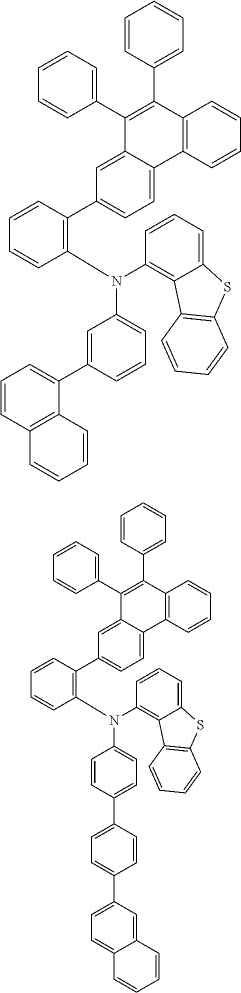 [SMILES: c1ccc(-c2c(-c3ccccc3)c3cc(-c4ccccc4N(c4cccc(-c5cccc6ccccc56)c4)c4cccc5sc6ccccc6c45)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc5sc6ccccc6c45)ccc3c3ccccc23)cc1]

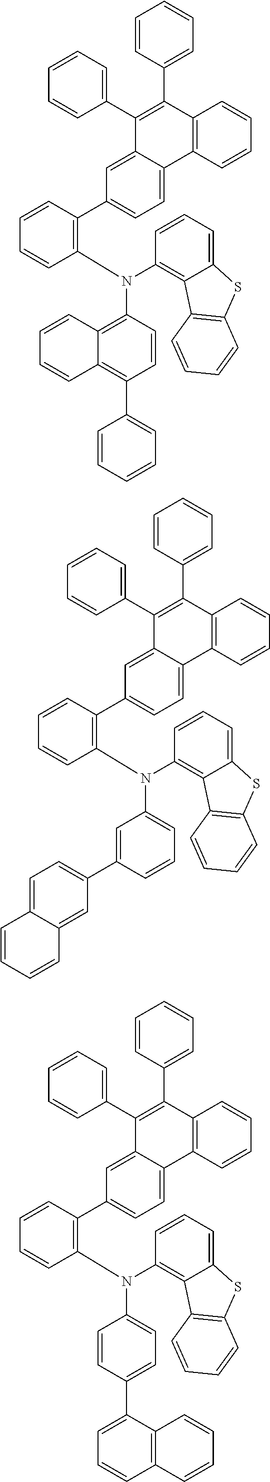

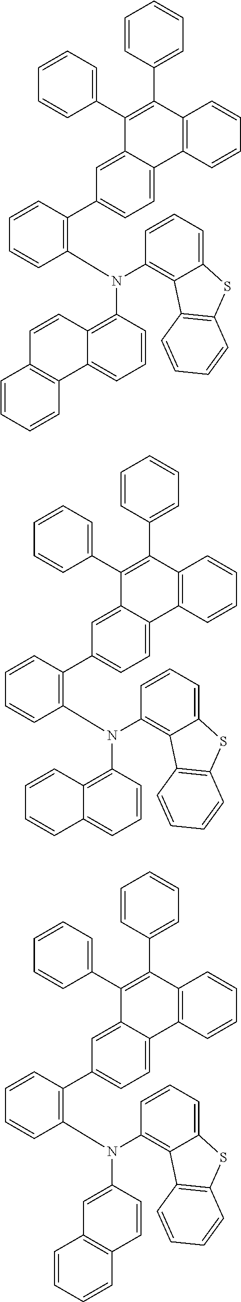 [SMILES: c1ccc(-c2c(-c3ccccc3)c3cc(-c4ccccc4N(c4cccc5c4ccc4ccccc45)c4cccc5sc6ccccc6c45)ccc3c3ccccc23)cc1 ; c1ccc(-c2c(-c3ccccc3)c3cc(-c4ccccc4N(c4cccc5ccccc45)c4cccc5sc6ccccc6c45)ccc3c3ccccc23)cc1 ; c1ccc(-c2c(-c3ccccc3)c3cc(-c4ccccc4N(c4ccc5ccccc5c4)c4cccc5sc6ccccc6c45)ccc3c3ccccc23)cc1]

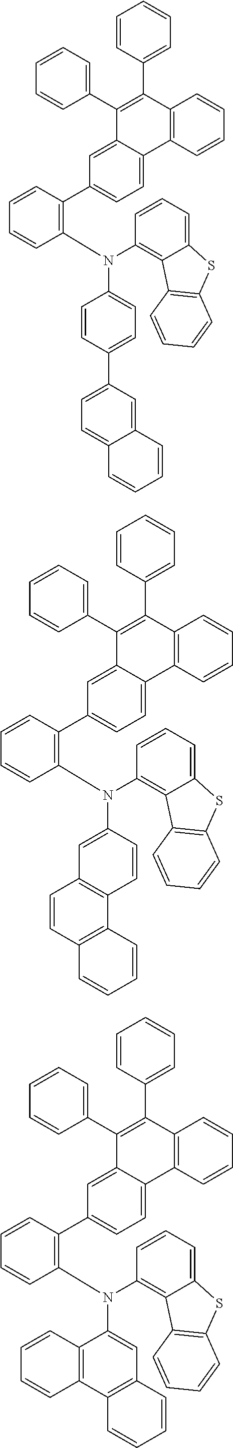

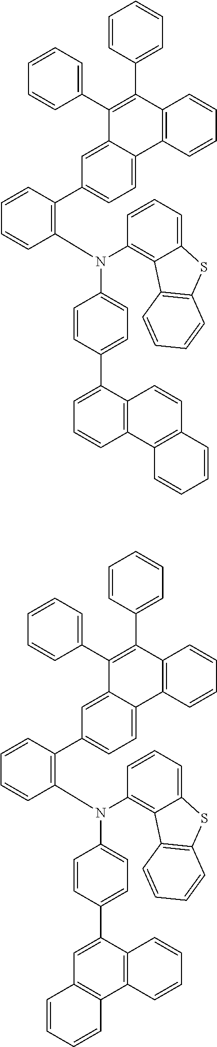 [SMILES: c1ccc(-c2c(-c3ccccc3)c3cc(-c4ccccc4N(c4ccc(-c5cccc6c5ccc5ccccc56)cc4)c4cccc5sc6ccccc6c45)ccc3c3ccccc23)cc1 ; c1ccc(-c2c(-c3ccccc3)c3cc(-c4ccccc4N(c4ccc(-c5cc6ccccc6c6ccccc56)cc4)c4cccc5sc6ccccc6c45)ccc3c3ccccc23)cc1]

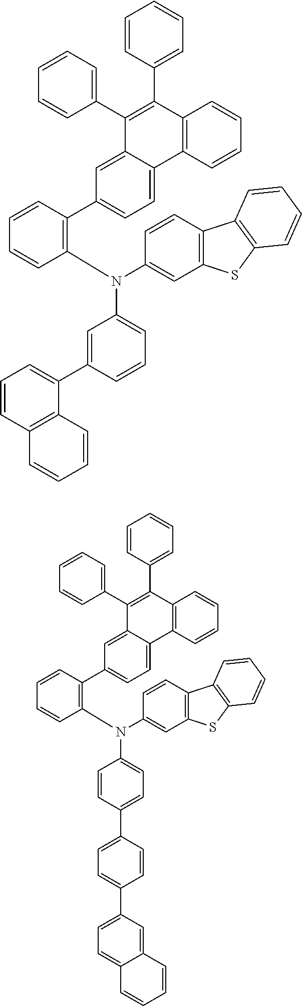 [SMILES: c1ccc(-c2c(-c3ccccc3)c3cc(-c4ccccc4N(c4cccc(-c5cccc6ccccc56)c4)c4ccc5c(c4)sc4ccccc45)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4ccc5c(c4)sc4ccccc45)ccc3c3ccccc23)cc1]

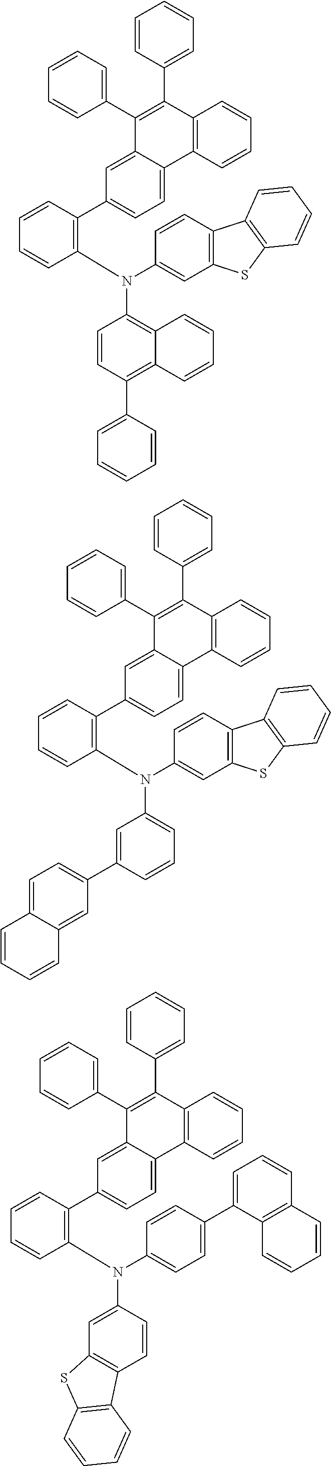

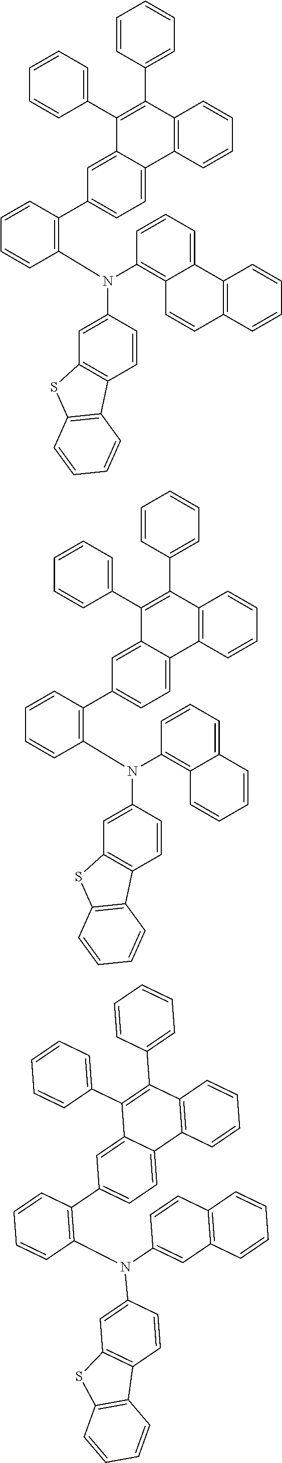

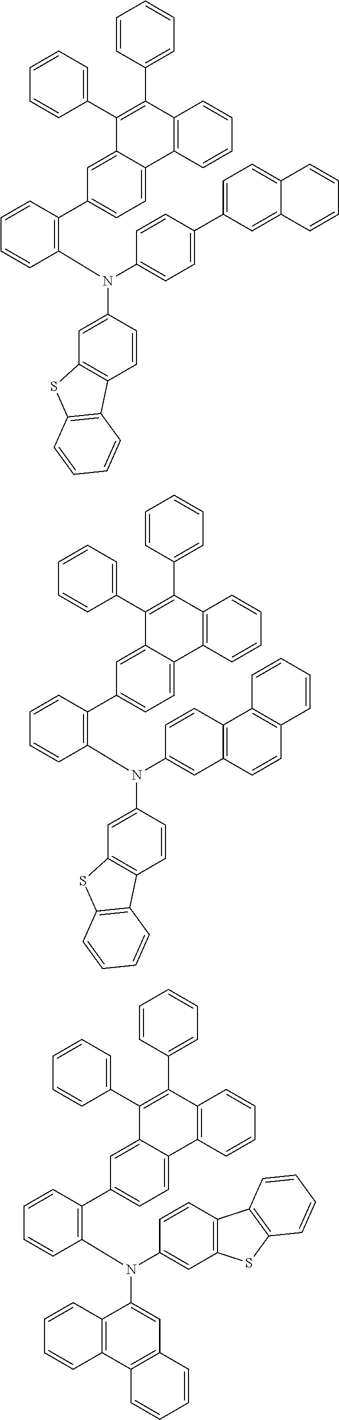

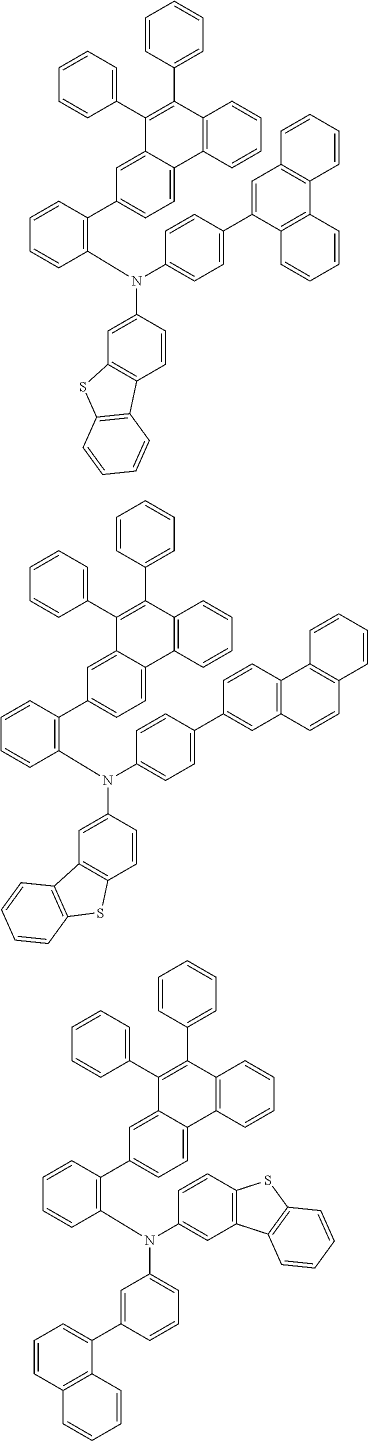

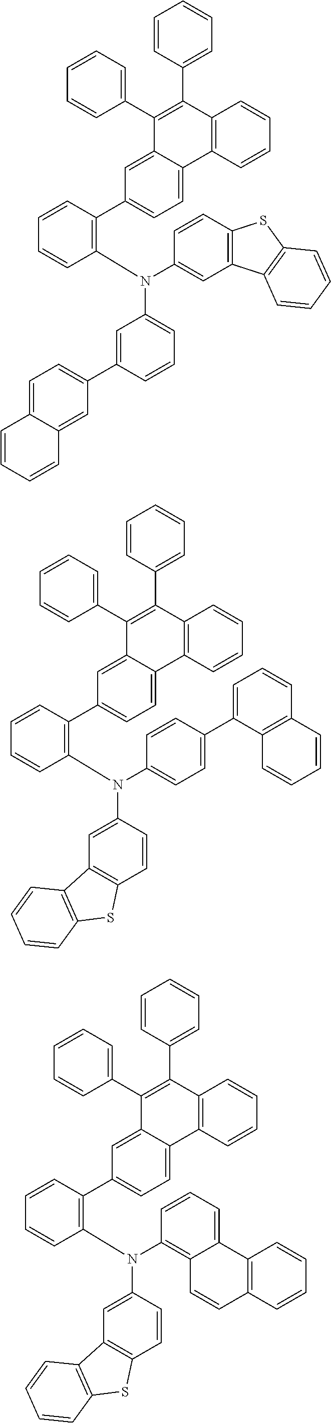 [SMILES: c1ccc(-c2c(-c3ccccc3)c3cc(-c4ccccc4N(c4cccc(-c5ccc6ccccc6c5)c4)c4ccc5sc6ccccc6c5c4)ccc3c3ccccc23)cc1 ; c1ccc(-c2c(-c3ccccc3)c3cc(-c4ccccc4N(c4ccc(-c5cccc6ccccc56)cc4)c4ccc5sc6ccccc6c5c4)ccc3c3ccccc23)cc1 ; c1ccc(-c2c(-c3ccccc3)c3cc(-c4ccccc4N(c4ccc5sc6ccccc6c5c4)c4cccc5c4ccc4ccccc45)ccc3c3ccccc23)cc1]

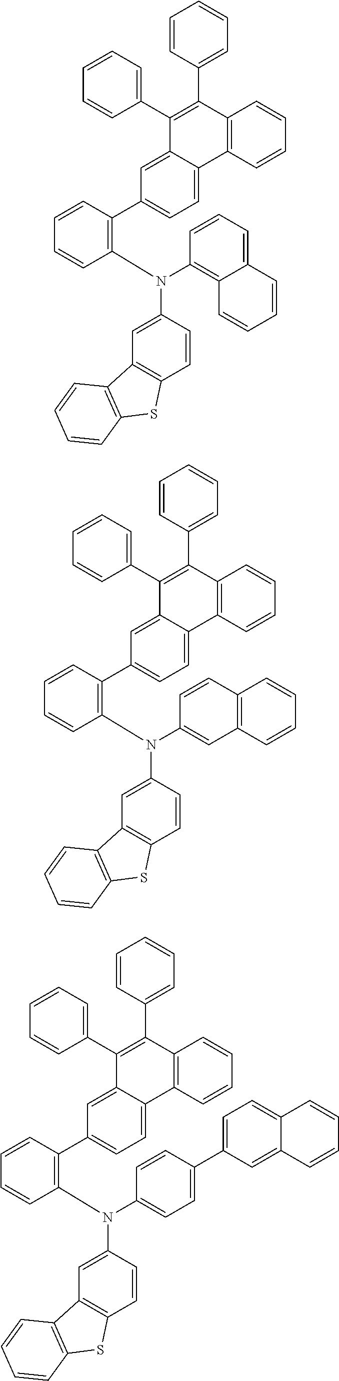 [SMILES: c1ccc(-c2c(-c3ccccc3)c3cc(-c4ccccc4N(c4ccc5sc6ccccc6c5c4)c4cccc5ccccc45)ccc3c3ccccc23)cc1 ; c1ccc(-c2c(-c3ccccc3)c3cc(-c4ccccc4N(c4ccc5ccccc5c4)c4ccc5sc6ccccc6c5c4)ccc3c3ccccc23)cc1 ; c1ccc(-c2c(-c3ccccc3)c3cc(-c4ccccc4N(c4ccc(-c5ccc6ccccc6c5)cc4)c4ccc5sc6ccccc6c5c4)ccc3c3ccccc23)cc1]

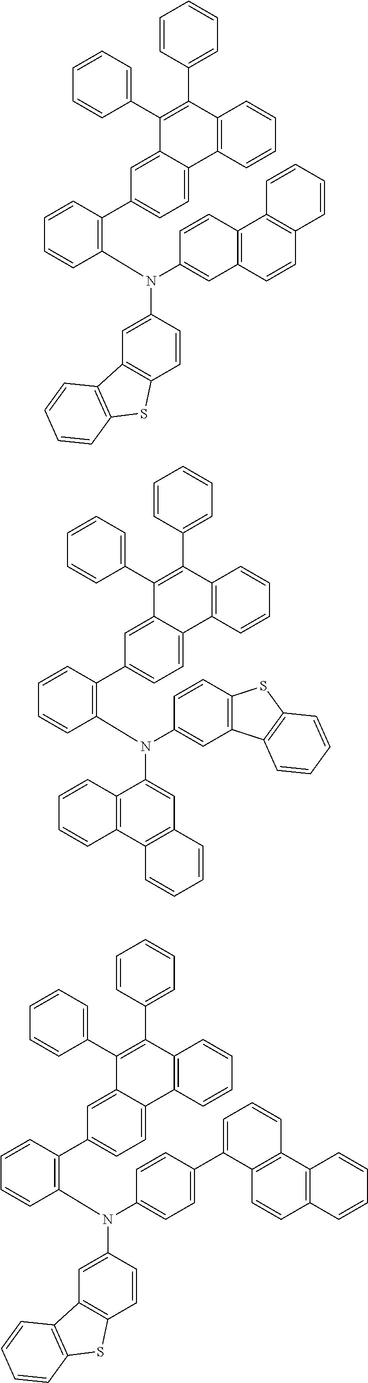 [SMILES: c1ccc(-c2c(-c3ccccc3)c3cc(-c4ccccc4N(c4ccc5c(ccc6ccccc65)c4)c4ccc5sc6ccccc6c5c4)ccc3c3ccccc23)cc1 ; c1ccc(-c2c(-c3ccccc3)c3cc(-c4ccccc4N(c4ccc5sc6ccccc6c5c4)c4cc5ccccc5c5ccccc45)ccc3c3ccccc23)cc1 ; c1ccc(-c2c(-c3ccccc3)c3cc(-c4ccccc4N(c4ccc(-c5cccc6c5ccc5ccccc56)cc4)c4ccc5sc6ccccc6c5c4)ccc3c3ccccc23)cc1]

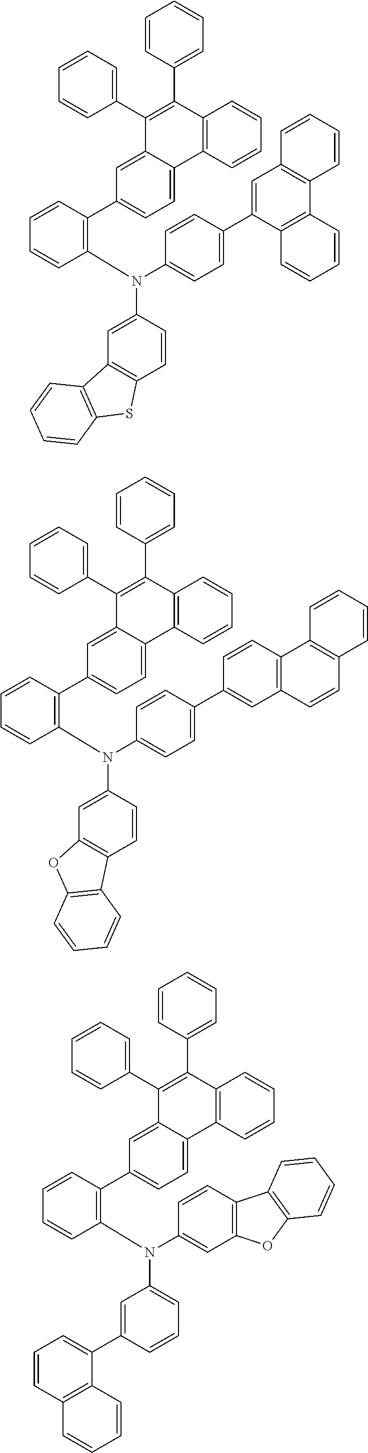 [SMILES: c1ccc(-c2c(-c3ccccc3)c3cc(-c4ccccc4N(c4ccc(-c5cc6ccccc6c6ccccc56)cc4)c4ccc5sc6ccccc6c5c4)ccc3c3ccccc23)cc1 ; c1ccc(-c2c(-c3ccccc3)c3cc(-c4ccccc4N(c4ccc(-c5ccc6c(ccc7ccccc76)c5)cc4)c4ccc5c(c4)oc4ccccc45)ccc3c3ccccc23)cc1 ; c1ccc(-c2c(-c3ccccc3)c3cc(-c4ccccc4N(c4cccc(-c5cccc6ccccc56)c4)c4ccc5c(c4)oc4ccccc45)ccc3c3ccccc23)cc1]

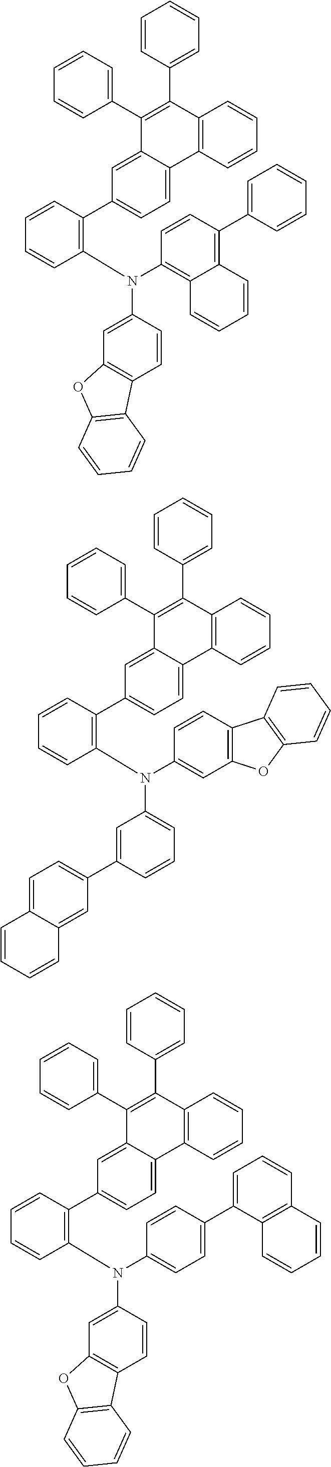 [SMILES: c1ccc(-c2ccc(N(c3ccc4c(c3)oc3ccccc34)c3ccccc3-c3ccc4c(c3)c(-c3ccccc3)c(-c3ccccc3)c3ccccc34)c3ccccc23)cc1 ; c1ccc(-c2c(-c3ccccc3)c3cc(-c4ccccc4N(c4cccc(-c5ccc6ccccc6c5)c4)c4ccc5c(c4)oc4ccccc45)ccc3c3ccccc23)cc1 ; c1ccc(-c2c(-c3ccccc3)c3cc(-c4ccccc4N(c4ccc(-c5cccc6ccccc56)cc4)c4ccc5c(c4)oc4ccccc45)ccc3c3ccccc23)cc1]

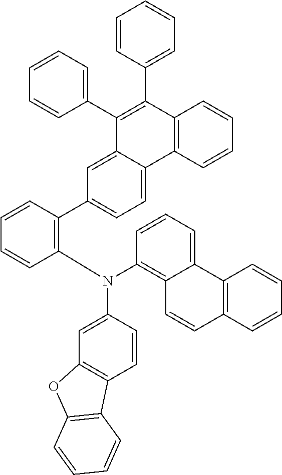 [SMILES: c1ccc(-c2c(-c3ccccc3)c3cc(-c4ccccc4N(c4ccc5c(c4)oc4ccccc45)c4cccc5c4ccc4ccccc45)ccc3c3ccccc23)cc1]

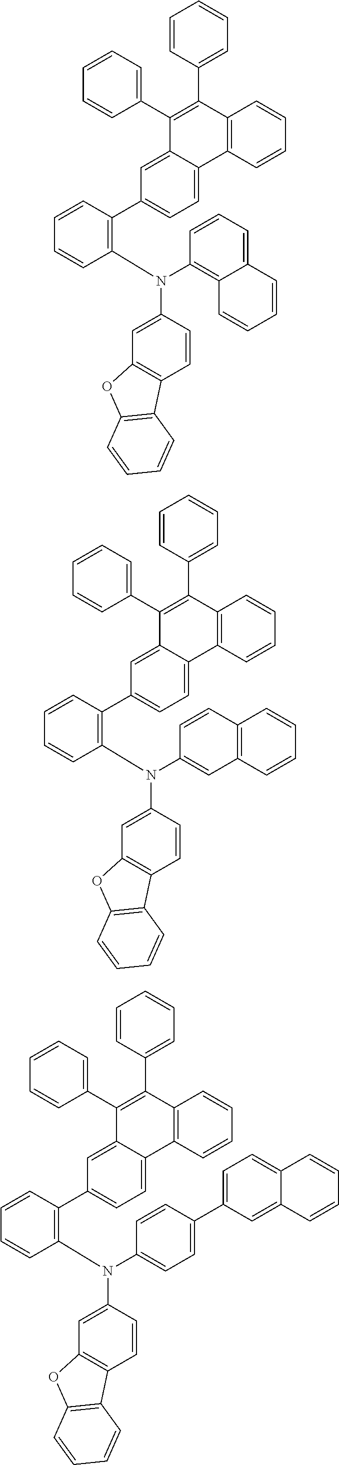

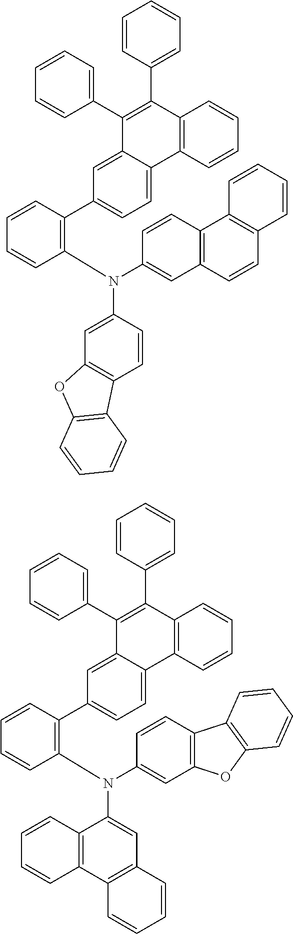

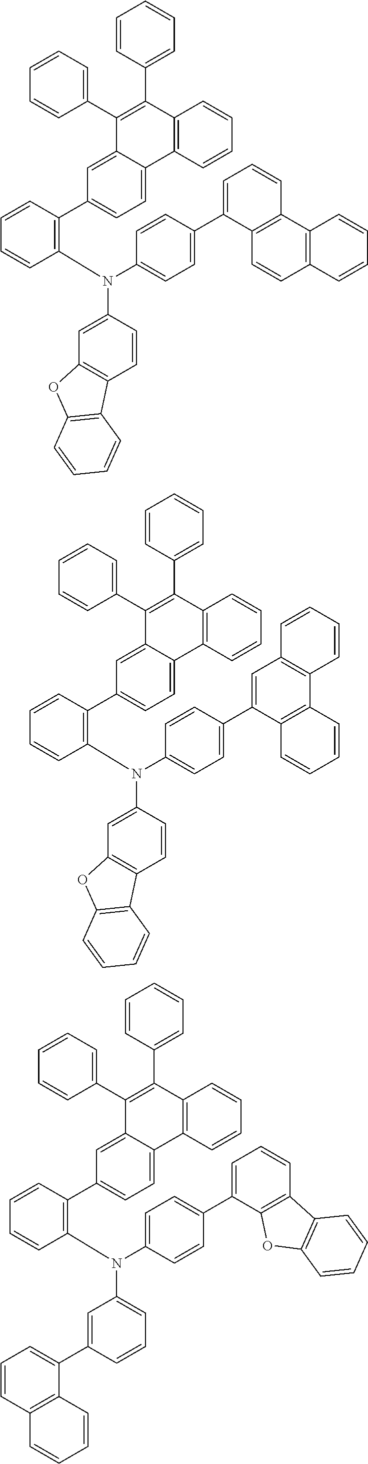

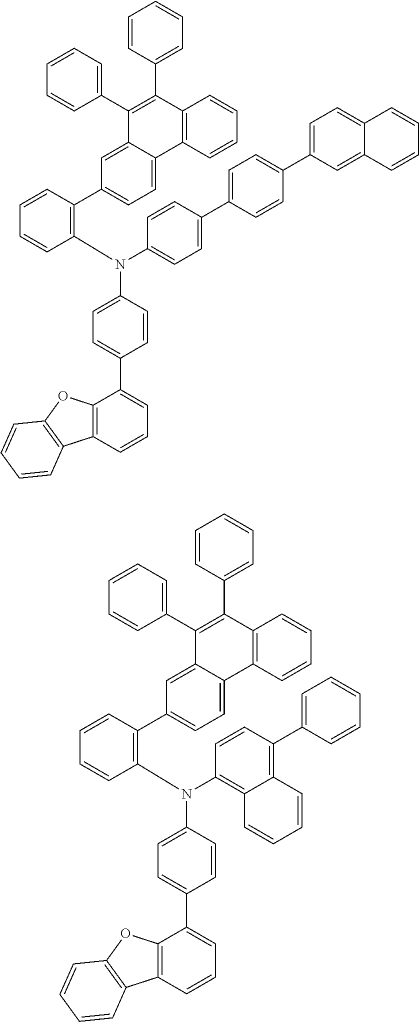 [SMILES: c1ccc(-c2c(-c3ccccc3)c3cc(-c4ccccc4N(c4ccc(-c5ccc(-c6ccc7ccccc7c6)cc5)cc4)c4ccc(-c5cccc6c5oc5ccccc56)cc4)ccc3c3ccccc23)cc1 ; c1ccc(-c2ccc(N(c3ccc(-c4cccc5c4oc4ccccc45)cc3)c3ccccc3-c3ccc4c(c3)c(-c3ccccc3)c(-c3ccccc3)c3ccccc34)c3ccccc23)cc1]

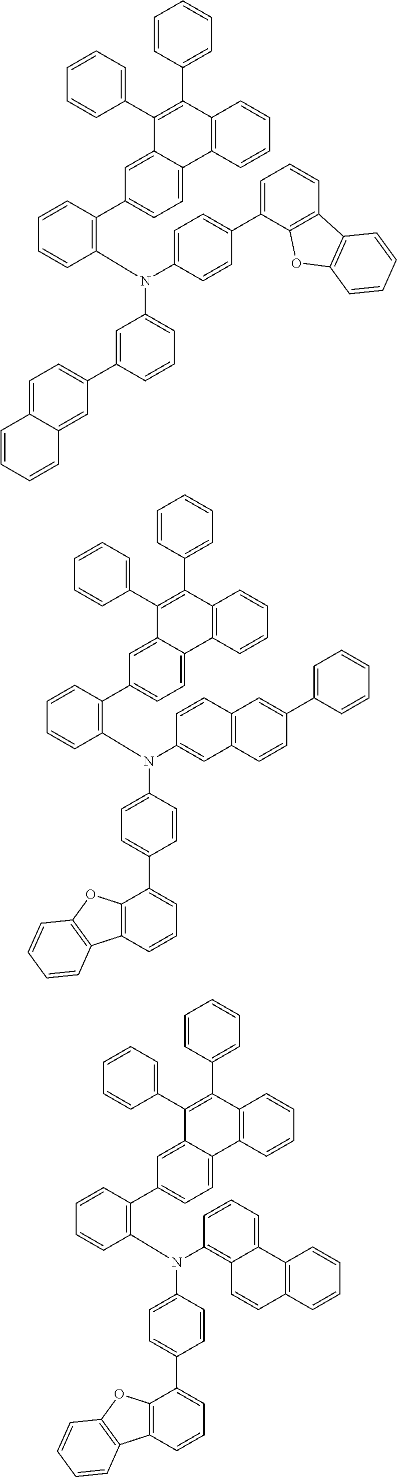 [SMILES: c1ccc(-c2c(-c3ccccc3)c3cc(-c4ccccc4N(c4ccc(-c5cccc6c5oc5ccccc56)cc4)c4cccc(-c5ccc6ccccc6c5)c4)ccc3c3ccccc23)cc1 ; c1ccc(-c2ccc3cc(N(c4ccc(-c5cccc6c5oc5ccccc56)cc4)c4ccccc4-c4ccc5c(c4)c(-c4ccccc4)c(-c4ccccc4)c4ccccc45)ccc3c2)cc1 ; c1ccc(-c2c(-c3ccccc3)c3cc(-c4ccccc4N(c4ccc(-c5cccc6c5oc5ccccc56)cc4)c4cccc5c4ccc4ccccc45)ccc3c3ccccc23)cc1]

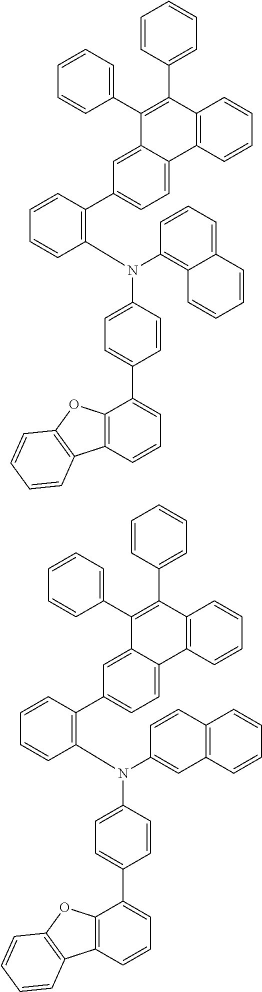 [SMILES: c1ccc(-c2c(-c3ccccc3)c3cc(-c4ccccc4N(c4ccc(-c5cccc6c5oc5ccccc56)cc4)c4cccc5ccccc45)ccc3c3ccccc23)cc1 ; c1ccc(-c2c(-c3ccccc3)c3cc(-c4ccccc4N(c4ccc(-c5cccc6c5oc5ccccc56)cc4)c4ccc5ccccc5c4)ccc3c3ccccc23)cc1]

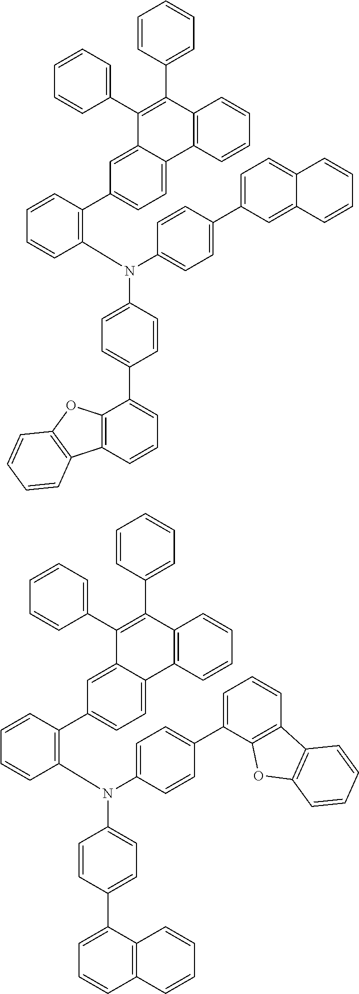 [SMILES: c1ccc(-c2c(-c3ccccc3)c3cc(-c4ccccc4N(c4ccc(-c5ccc6ccccc6c5)cc4)c4ccc(-c5cccc6c5oc5ccccc56)cc4)ccc3c3ccccc23)cc1 ; c1ccc(-c2c(-c3ccccc3)c3cc(-c4ccccc4N(c4ccc(-c5cccc6ccccc56)cc4)c4ccc(-c5cccc6c5oc5ccccc56)cc4)ccc3c3ccccc23)cc1]

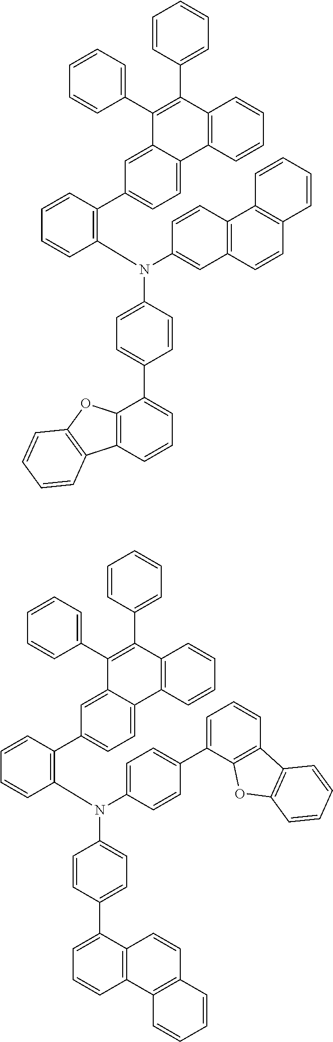 [SMILES: c1ccc(-c2c(-c3ccccc3)c3cc(-c4ccccc4N(c4ccc(-c5cccc6c5oc5ccccc56)cc4)c4ccc5c(ccc6ccccc65)c4)ccc3c3ccccc23)cc1 ; c1ccc(-c2c(-c3ccccc3)c3cc(-c4ccccc4N(c4ccc(-c5cccc6c5ccc5ccccc56)cc4)c4ccc(-c5cccc6c5oc5ccccc56)cc4)ccc3c3ccccc23)cc1]

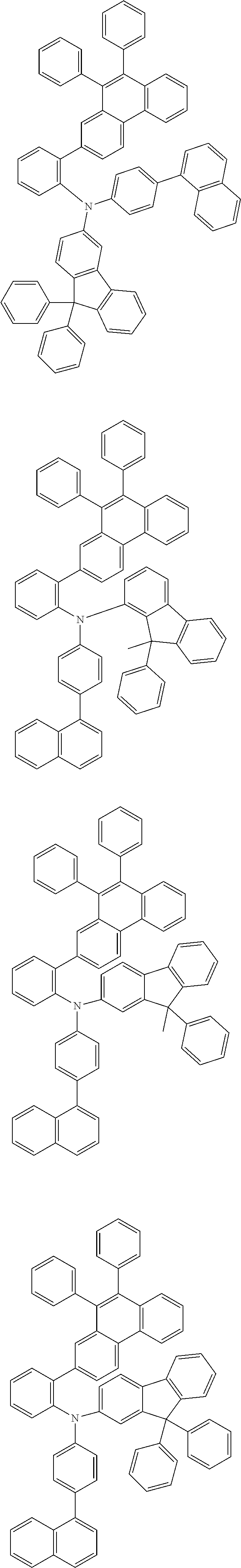 [SMILES: c1ccc(-c2c(-c3ccccc3)c3cc(-c4ccccc4N(c4ccc(-c5cccc6ccccc56)cc4)c4ccc5c(c4)-c4ccccc4C5(c4ccccc4)c4ccccc4)ccc3c3ccccc23)cc1 ; CC1(c2ccccc2)c2ccccc2-c2cccc(N(c3ccc(-c4cccc5ccccc45)cc3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4cccc5ccccc45)cc3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4ccc(-c5cccc6ccccc56)cc4)c4ccc5c(c4)C(c4ccccc4)(c4ccccc4)c4ccccc4-5)ccc3c3ccccc23)cc1]

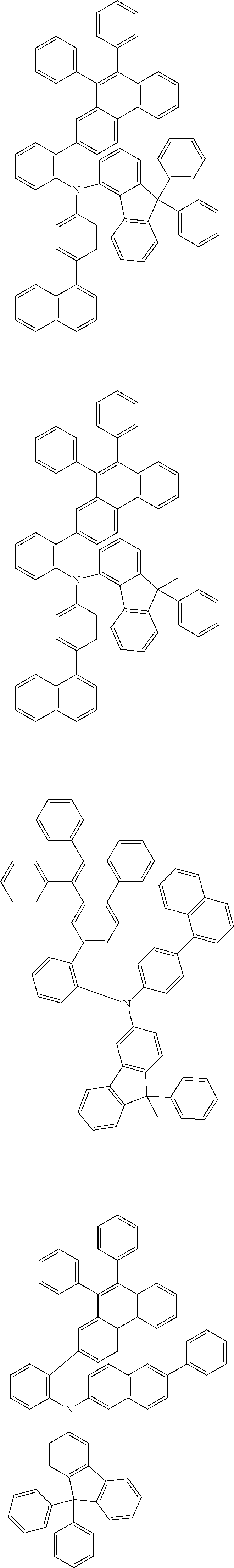 [SMILES: c1ccc(-c2c(-c3ccccc3)c3cc(-c4ccccc4N(c4ccc(-c5cccc6ccccc56)cc4)c4cccc5c4-c4ccccc4C5(c4ccccc4)c4ccccc4)ccc3c3ccccc23)cc1 ; CC1(c2ccccc2)c2ccccc2-c2c(N(c3ccc(-c4cccc5ccccc45)cc3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4cccc5ccccc45)cc3)c3ccccc3-c3ccc4c(c3)c(-c3ccccc3)c(-c3ccccc3)c3ccccc34)ccc21 ; c1ccc(-c2ccc3cc(N(c4ccc5c(c4)-c4ccccc4C5(c4ccccc4)c4ccccc4)c4ccccc4-c4ccc5c(c4)c(-c4ccccc4)c(-c4ccccc4)c4ccccc45)ccc3c2)cc1]

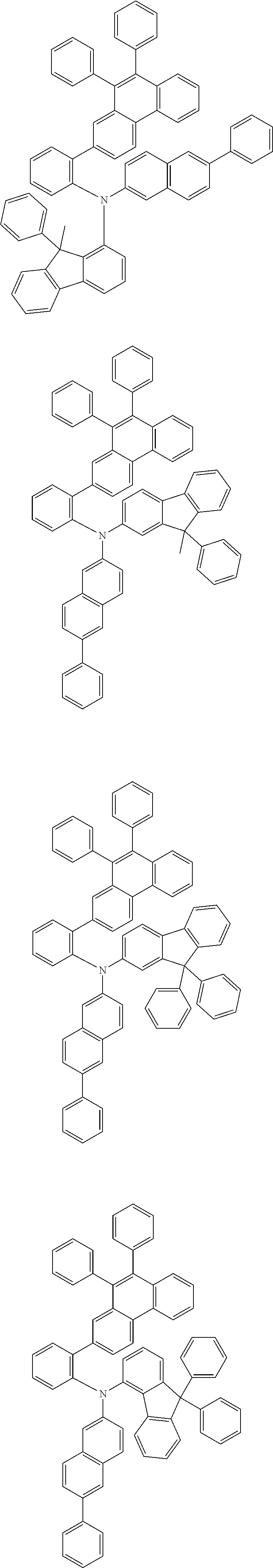 [SMILES: CC1(c2ccccc2)c2ccccc2-c2cccc(N(c3ccc4cc(-c5ccccc5)ccc4c3)c3ccccc3-c3ccc4c(c3)c(-c3ccccc3)c(-c3ccccc3)c3ccccc34)c21 ; CC1(c2ccccc2)c2ccccc2-c2ccc(N(c3ccc4cc(-c5ccccc5)ccc4c3)c3ccccc3-c3ccc4c(c3)c(-c3ccccc3)c(-c3ccccc3)c3ccccc34)cc21 ; c1ccc(-c2ccc3cc(N(c4ccc5c(c4)C(c4ccccc4)(c4ccccc4)c4ccccc4-5)c4ccccc4-c4ccc5c(c4)c(-c4ccccc4)c(-c4ccccc4)c4ccccc45)ccc3c2)cc1 ; c1ccc(-c2ccc3cc(N(c4ccccc4-c4ccc5c(c4)c(-c4ccccc4)c(-c4ccccc4)c4ccccc45)c4cccc5c4-c4ccccc4C5(c4ccccc4)c4ccccc4)ccc3c2)cc1]

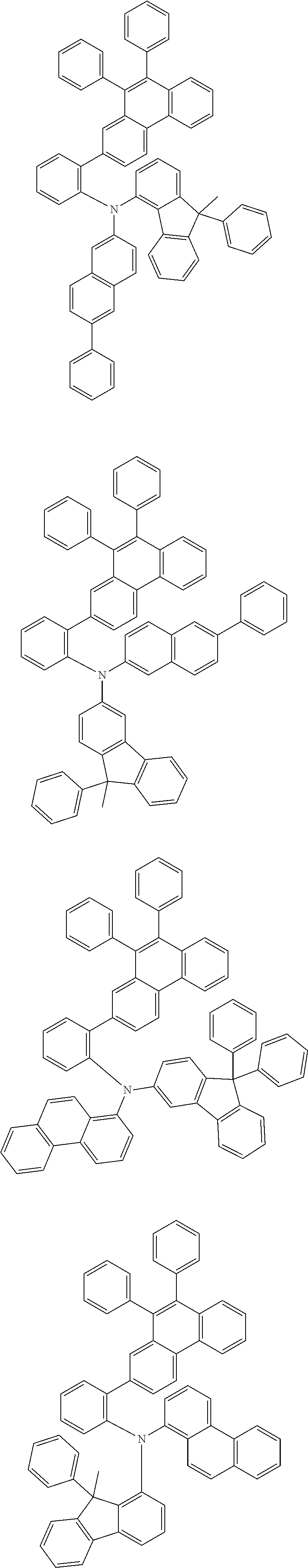 [SMILES: CC1(c2ccccc2)c2ccccc2-c2c(N(c3ccc4cc(-c5ccccc5)ccc4c3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc4cc(-c5ccccc5)ccc4c3)c3ccccc3-c3ccc4c(c3)c(-c3ccccc3)c(-c3ccccc3)c3ccccc34)ccc21 ; c1ccc(-c2c(-c3ccccc3)c3cc(-c4ccccc4N(c4ccc5c(c4)-c4ccccc4C5(c4ccccc4)c4ccccc4)c4cccc5c4ccc4ccccc45)ccc3c3ccccc23)cc1 ; CC1(c2ccccc2)c2ccccc2-c2cccc(N(c3ccccc3-c3ccc4c(c3)c(-c3ccccc3)c(-c3ccccc3)c3ccccc34)c3cccc4c3ccc3ccccc34)c21]

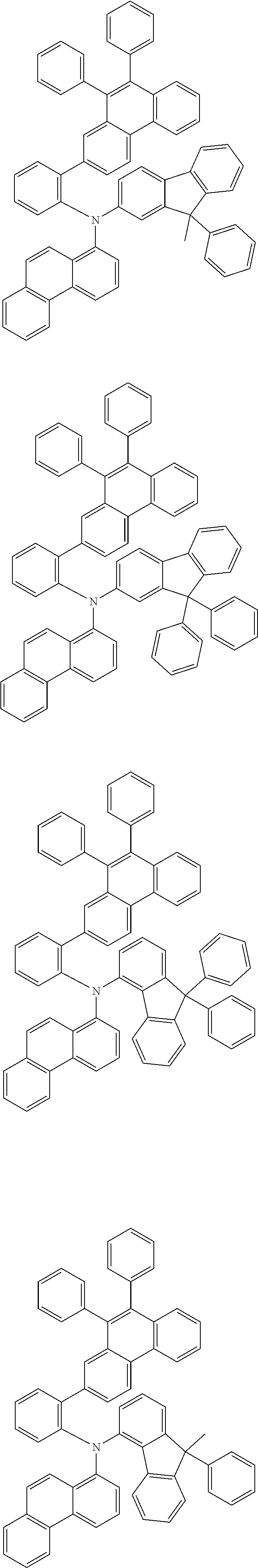 [SMILES: CC1(c2ccccc2)c2ccccc2-c2ccc(N(c3ccccc3-c3ccc4c(c3)c(-c3ccccc3)c(-c3ccccc3)c3ccccc34)c3cccc4c3ccc3ccccc34)cc21 ; c1ccc(-c2c(-c3ccccc3)c3cc(-c4ccccc4N(c4ccc5c(c4)C(c4ccccc4)(c4ccccc4)c4ccccc4-5)c4cccc5c4ccc4ccccc45)ccc3c3ccccc23)cc1 ; c1ccc(-c2c(-c3ccccc3)c3cc(-c4ccccc4N(c4cccc5c4-c4ccccc4C5(c4ccccc4)c4ccccc4)c4cccc5c4ccc4ccccc45)ccc3c3ccccc23)cc1 ; CC1(c2ccccc2)c2ccccc2-c2c(N(c3ccccc3-c3ccc4c(c3)c(-c3ccccc3)c(-c3ccccc3)c3ccccc34)c3cccc4c3ccc3ccccc34)cccc21]

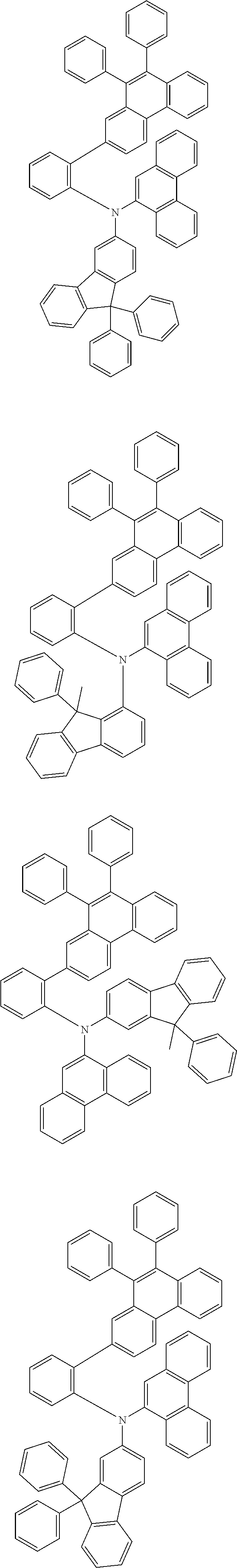 [SMILES: c1ccc(-c2c(-c3ccccc3)c3cc(-c4ccccc4N(c4ccc5c(c4)-c4ccccc4C5(c4ccccc4)c4ccccc4)c4cc5ccccc5c5ccccc45)ccc3c3ccccc23)cc1 ; CC1(c2ccccc2)c2ccccc2-c2cccc(N(c3ccccc3-c3ccc4c(c3)c(-c3ccccc3)c(-c3ccccc3)c3ccccc34)c3cc4ccccc4c4ccccc34)c21 ; CC1(c2ccccc2)c2ccccc2-c2ccc(N(c3ccccc3-c3ccc4c(c3)c(-c3ccccc3)c(-c3ccccc3)c3ccccc34)c3cc4ccccc4c4ccccc34)cc21 ; c1ccc(-c2c(-c3ccccc3)c3cc(-c4ccccc4N(c4ccc5c(c4)C(c4ccccc4)(c4ccccc4)c4ccccc4-5)c4cc5ccccc5c5ccccc45)ccc3c3ccccc23)cc1]

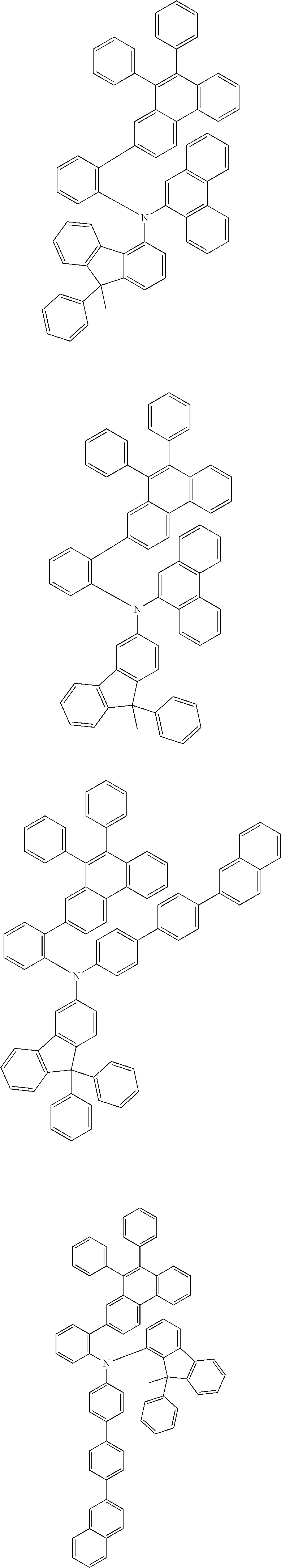 [SMILES: CC1(c2ccccc2)c2ccccc2-c2c(N(c3ccccc3-c3ccc4c(c3)c(-c3ccccc3)c(-c3ccccc3)c3ccccc34)c3cc4ccccc4c4ccccc34)cccc21 ; CC1(c2ccccc2)c2ccccc2-c2cc(N(c3ccccc3-c3ccc4c(c3)c(-c3ccccc3)c(-c3ccccc3)c3ccccc34)c3cc4ccccc4c4ccccc34)ccc21 ; c1ccc(-c2c(-c3ccccc3)c3cc(-c4ccccc4N(c4ccc(-c5ccc(-c6ccc7ccccc7c6)cc5)cc4)c4ccc5c(c4)-c4ccccc4C5(c4ccccc4)c4ccccc4)ccc3c3ccccc23)cc1 ; CC1(c2ccccc2)c2ccccc2-c2cccc(N(c3ccc(-c4ccc(-c5ccc6ccccc6c5)cc4)cc3)c3ccccc3-c3ccc4c(c3)c(-c3ccccc3)c(-c3ccccc3)c3ccccc34)c21]

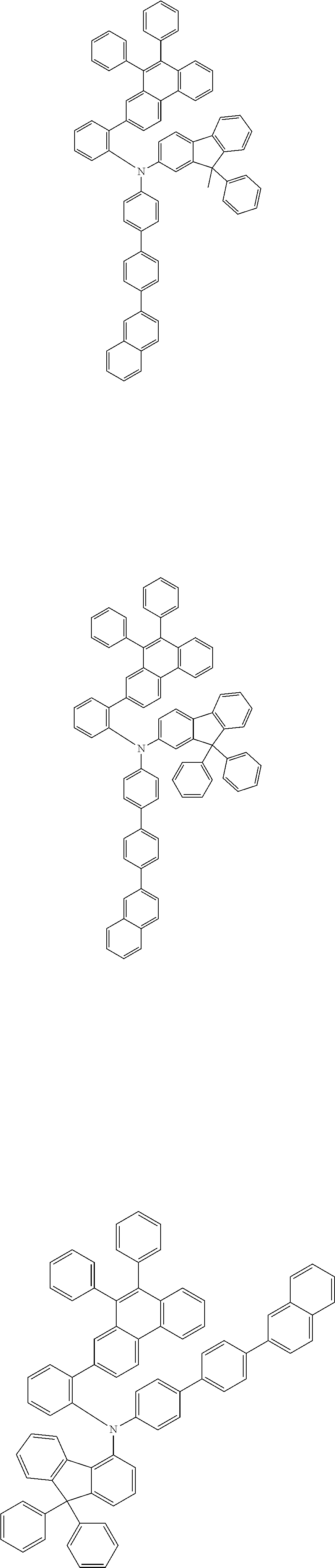 [SMILES: CC1(c2ccccc2)c2ccccc2-c2ccc(N(c3ccc(-c4ccc(-c5ccc6ccccc6c5)cc4)cc3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4ccc(-c5ccc(-c6ccc7ccccc7c6)cc5)cc4)c4ccc5c(c4)C(c4ccccc4)(c4ccccc4)c4ccccc4-5)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc5c4-c4ccccc4C5(c4ccccc4)c4ccccc4)ccc3c3ccccc23)cc1]

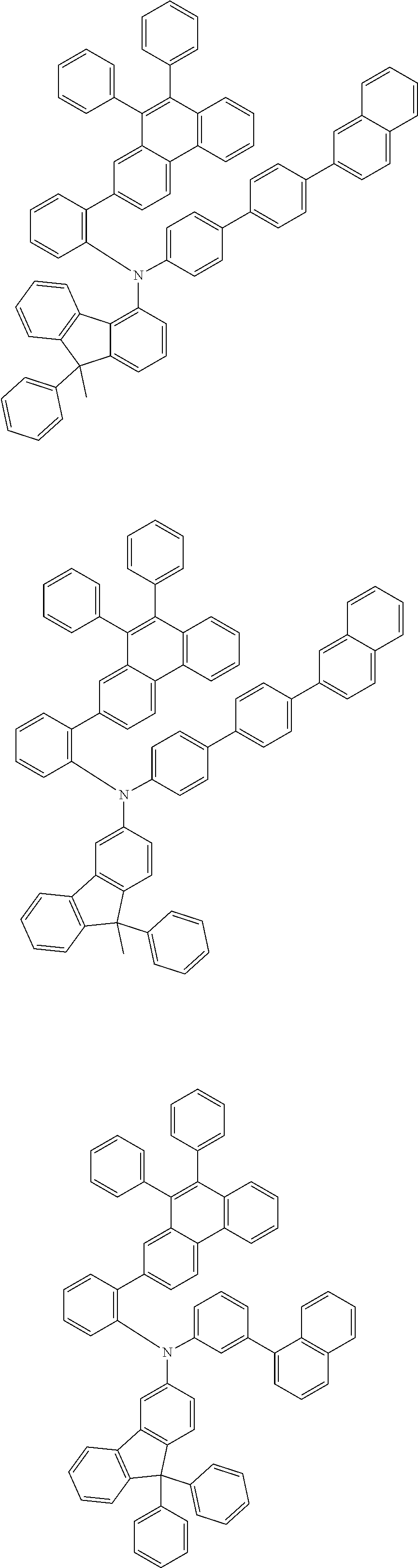 [SMILES: CC1(c2ccccc2)c2ccccc2-c2c(N(c3ccc(-c4ccc(-c5ccc6ccccc6c5)cc4)cc3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4ccc(-c5ccc6ccccc6c5)cc4)cc3)c3ccccc3-c3ccc4c(c3)c(-c3ccccc3)c(-c3ccccc3)c3ccccc34)ccc21 ; c1ccc(-c2c(-c3ccccc3)c3cc(-c4ccccc4N(c4cccc(-c5cccc6ccccc56)c4)c4ccc5c(c4)-c4ccccc4C5(c4ccccc4)c4ccccc4)ccc3c3ccccc23)cc1]

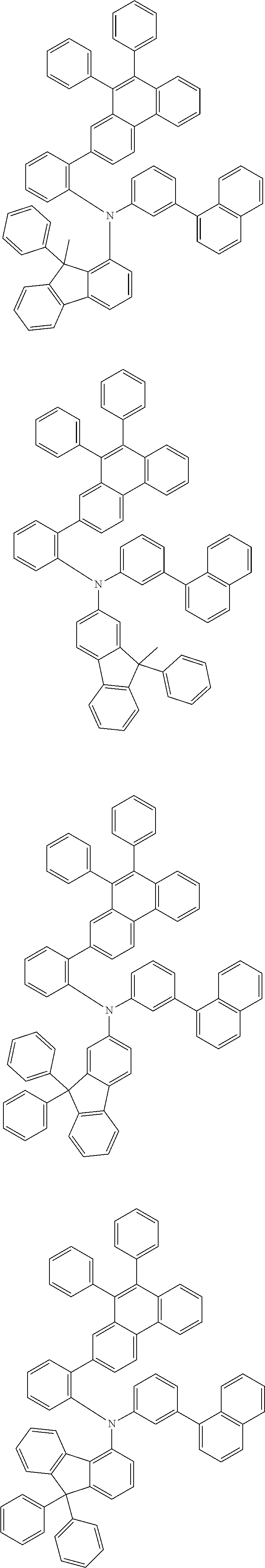 [SMILES: CC1(c2ccccc2)c2ccccc2-c2cccc(N(c3cccc(-c4cccc5ccccc45)c3)c3ccccc3-c3ccc4c(c3)c(-c3ccccc3)c(-c3ccccc3)c3ccccc34)c21 ; CC1(c2ccccc2)c2ccccc2-c2ccc(N(c3cccc(-c4cccc5ccccc45)c3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4cccc(-c5cccc6ccccc56)c4)c4ccc5c(c4)C(c4ccccc4)(c4ccccc4)c4ccccc4-5)ccc3c3ccccc23)cc1 ; c1ccc(-c2c(-c3ccccc3)c3cc(-c4ccccc4N(c4cccc(-c5cccc6ccccc56)c4)c4cccc5c4-c4ccccc4C5(c4ccccc4)c4ccccc4)ccc3c3ccccc23)cc1]

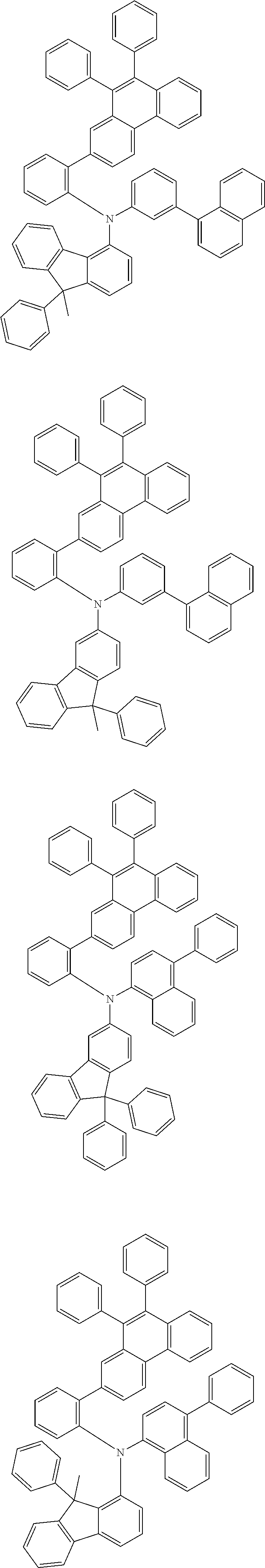 [SMILES: CC1(c2ccccc2)c2ccccc2-c2c(N(c3cccc(-c4cccc5ccccc45)c3)c3ccccc3-c3ccc4c(c3)c(-c3ccccc3)c(-c3ccccc3)c3ccccc34)cccc21 ; CC1(c2ccccc2)c2ccccc2-c2cc(N(c3cccc(-c4cccc5ccccc45)c3)c3ccccc3-c3ccc4c(c3)c(-c3ccccc3)c(-c3ccccc3)c3ccccc34)ccc21 ; c1ccc(-c2ccc(N(c3ccc4c(c3)-c3ccccc3C4(c3ccccc3)c3ccccc3)c3ccccc3-c3ccc4c(c3)c(-c3ccccc3)c(-c3ccccc3)c3ccccc34)c3ccccc23)cc1 ; CC1(c2ccccc2)c2ccccc2-c2cccc(N(c3ccccc3-c3ccc4c(c3)c(-c3ccccc3)c(-c3ccccc3)c3ccccc34)c3ccc(-c4ccccc4)c4ccccc34)c21]

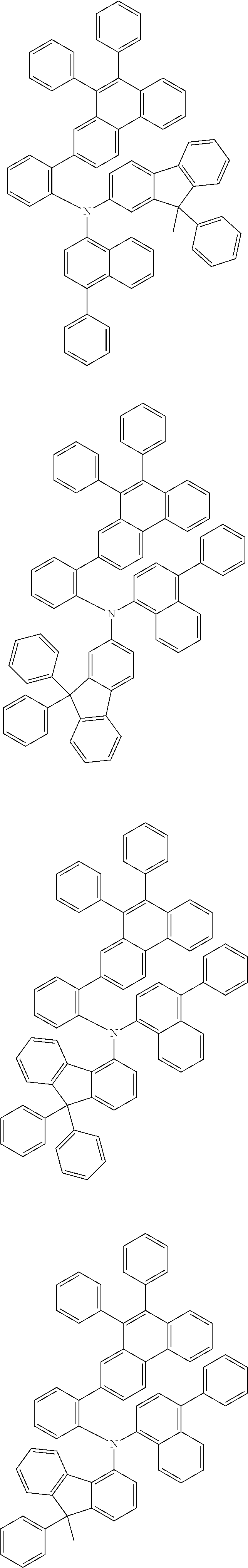 [SMILES: CC1(c2ccccc2)c2ccccc2-c2ccc(N(c3ccccc3-c3ccc4c(c3)c(-c3ccccc3)c(-c3ccccc3)c3ccccc34)c3ccc(-c4ccccc4)c4ccccc34)cc21 ; c1ccc(-c2ccc(N(c3ccc4c(c3)C(c3ccccc3)(c3ccccc3)c3ccccc3-4)c3ccccc3-c3ccc4c(c3)c(-c3ccccc3)c(-c3ccccc3)c3ccccc34)c3ccccc23)cc1 ; c1ccc(-c2ccc(N(c3ccccc3-c3ccc4c(c3)c(-c3ccccc3)c(-c3ccccc3)c3ccccc34)c3cccc4c3-c3ccccc3C4(c3ccccc3)c3ccccc3)c3ccccc23)cc1 ; CC1(c2ccccc2)c2ccccc2-c2c(N(c3ccccc3-c3ccc4c(c3)c(-c3ccccc3)c(-c3ccccc3)c3ccccc34)c3ccc(-c4ccccc4)c4ccccc34)cccc21]

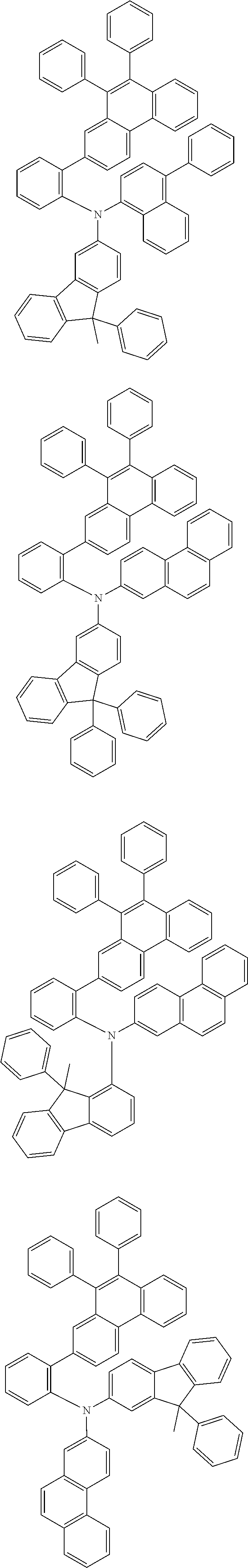

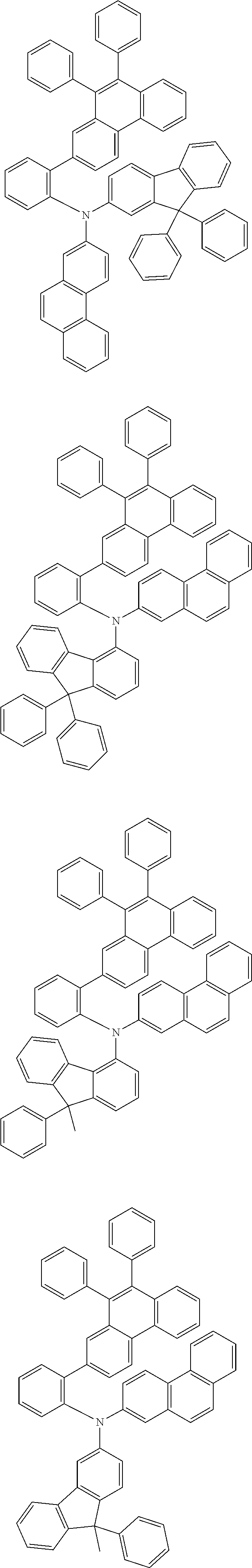 [SMILES: c1ccc(-c2c(-c3ccccc3)c3cc(-c4ccccc4N(c4ccc5c(c4)C(c4ccccc4)(c4ccccc4)c4ccccc4-5)c4ccc5c(ccc6ccccc65)c4)ccc3c3ccccc23)cc1 ; c1ccc(-c2c(-c3ccccc3)c3cc(-c4ccccc4N(c4ccc5c(ccc6ccccc65)c4)c4cccc5c4-c4ccccc4C5(c4ccccc4)c4ccccc4)ccc3c3ccccc23)cc1 ; CC1(c2ccccc2)c2ccccc2-c2c(N(c3ccc4c(ccc5ccccc54)c3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc4c(ccc5ccccc54)c3)c3ccccc3-c3ccc4c(c3)c(-c3ccccc3)c(-c3ccccc3)c3ccccc34)ccc21]

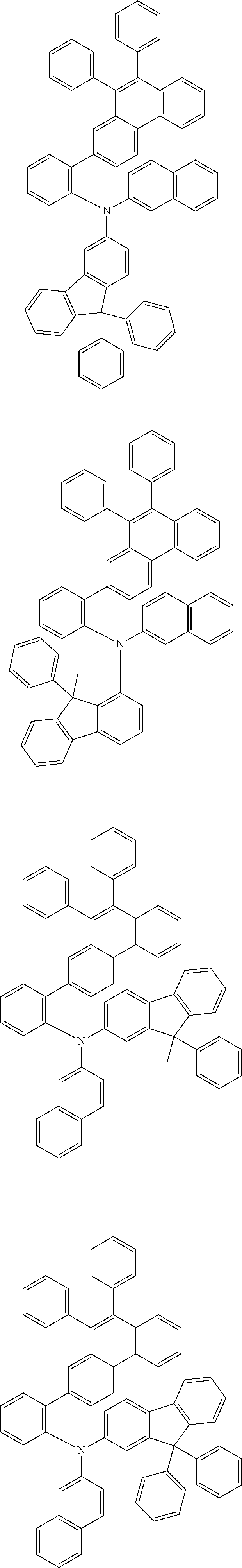 [SMILES: c1ccc(-c2c(-c3ccccc3)c3cc(-c4ccccc4N(c4ccc5c(c4)-c4ccccc4C5(c4ccccc4)c4ccccc4)c4ccc5ccccc5c4)ccc3c3ccccc23)cc1 ; CC1(c2ccccc2)c2ccccc2-c2cccc(N(c3ccc4ccccc4c3)c3ccccc3-c3ccc4c(c3)c(-c3ccccc3)c(-c3ccccc3)c3ccccc34)c21 ; CC1(c2ccccc2)c2ccccc2-c2ccc(N(c3ccc4ccccc4c3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4ccc5c(c4)C(c4ccccc4)(c4ccccc4)c4ccccc4-5)c4ccc5ccccc5c4)ccc3c3ccccc23)cc1]

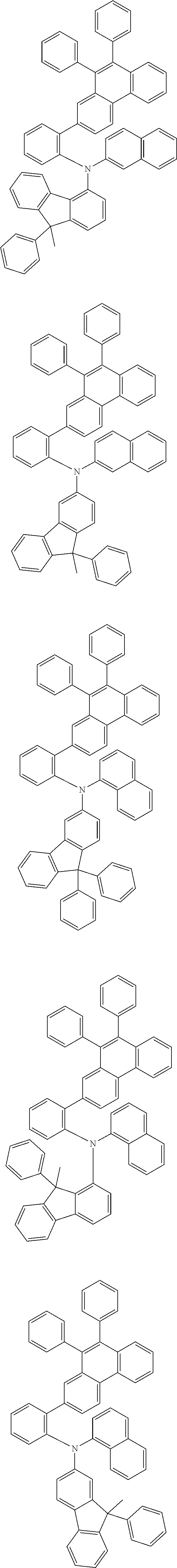

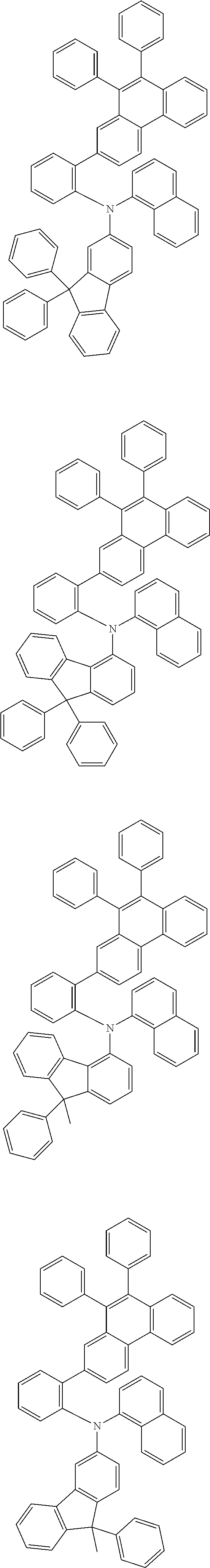 [SMILES: c1ccc(-c2c(-c3ccccc3)c3cc(-c4ccccc4N(c4ccc5c(c4)C(c4ccccc4)(c4ccccc4)c4ccccc4-5)c4cccc5ccccc45)ccc3c3ccccc23)cc1 ; c1ccc(-c2c(-c3ccccc3)c3cc(-c4ccccc4N(c4cccc5c4-c4ccccc4C5(c4ccccc4)c4ccccc4)c4cccc5ccccc45)ccc3c3ccccc23)cc1 ; CC1(c2ccccc2)c2ccccc2-c2c(N(c3ccccc3-c3ccc4c(c3)c(-c3ccccc3)c(-c3ccccc3)c3ccccc34)c3cccc4ccccc34)cccc21 ; CC1(c2ccccc2)c2ccccc2-c2cc(N(c3ccccc3-c3ccc4c(c3)c(-c3ccccc3)c(-c3ccccc3)c3ccccc34)c3cccc4ccccc34)ccc21]

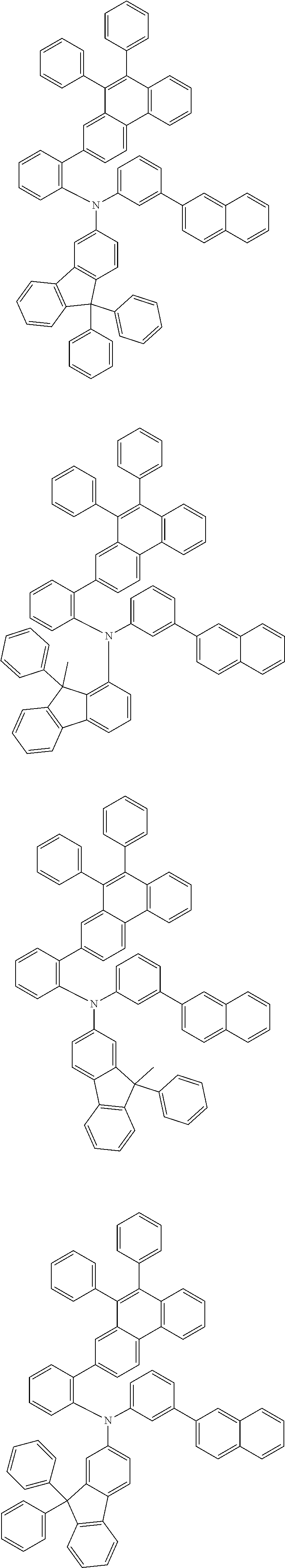 [SMILES: c1ccc(-c2c(-c3ccccc3)c3cc(-c4ccccc4N(c4cccc(-c5ccc6ccccc6c5)c4)c4ccc5c(c4)-c4ccccc4C5(c4ccccc4)c4ccccc4)ccc3c3ccccc23)cc1 ; CC1(c2ccccc2)c2ccccc2-c2cccc(N(c3cccc(-c4ccc5ccccc5c4)c3)c3ccccc3-c3ccc4c(c3)c(-c3ccccc3)c(-c3ccccc3)c3ccccc34)c21 ; CC1(c2ccccc2)c2ccccc2-c2ccc(N(c3cccc(-c4ccc5ccccc5c4)c3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4cccc(-c5ccc6ccccc6c5)c4)c4ccc5c(c4)C(c4ccccc4)(c4ccccc4)c4ccccc4-5)ccc3c3ccccc23)cc1]

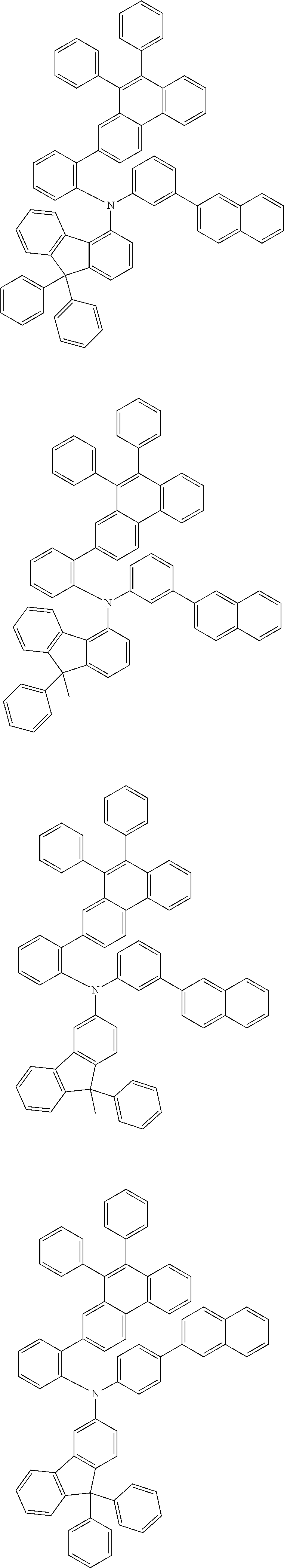 [SMILES: c1ccc(-c2c(-c3ccccc3)c3cc(-c4ccccc4N(c4cccc(-c5ccc6ccccc6c5)c4)c4cccc5c4-c4ccccc4C5(c4ccccc4)c4ccccc4)ccc3c3ccccc23)cc1 ; CC1(c2ccccc2)c2ccccc2-c2c(N(c3cccc(-c4ccc5ccccc5c4)c3)c3ccccc3-c3ccc4c(c3)c(-c3ccccc3)c(-c3ccccc3)c3ccccc34)cccc21 ; CC1(c2ccccc2)c2ccccc2-c2cc(N(c3cccc(-c4ccc5ccccc5c4)c3)c3ccccc3-c3ccc4c(c3)c(-c3ccccc3)c(-c3ccccc3)c3ccccc34)ccc21 ; c1ccc(-c2c(-c3ccccc3)c3cc(-c4ccccc4N(c4ccc(-c5ccc6ccccc6c5)cc4)c4ccc5c(c4)-c4ccccc4C5(c4ccccc4)c4ccccc4)ccc3c3ccccc23)cc1]

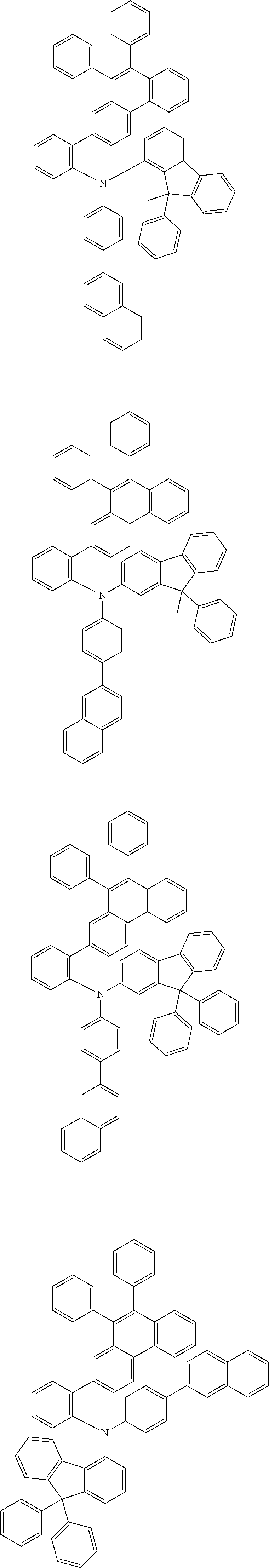 [SMILES: CC1(c2ccccc2)c2ccccc2-c2cccc(N(c3ccc(-c4ccc5ccccc5c4)cc3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4ccc5ccccc5c4)cc3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4ccc(-c5ccc6ccccc6c5)cc4)c4ccc5c(c4)C(c4ccccc4)(c4ccccc4)c4ccccc4-5)ccc3c3ccccc23)cc1 ; c1ccc(-c2c(-c3ccccc3)c3cc(-c4ccccc4N(c4ccc(-c5ccc6ccccc6c5)cc4)c4cccc5c4-c4ccccc4C5(c4ccccc4)c4ccccc4)ccc3c3ccccc23)cc1]

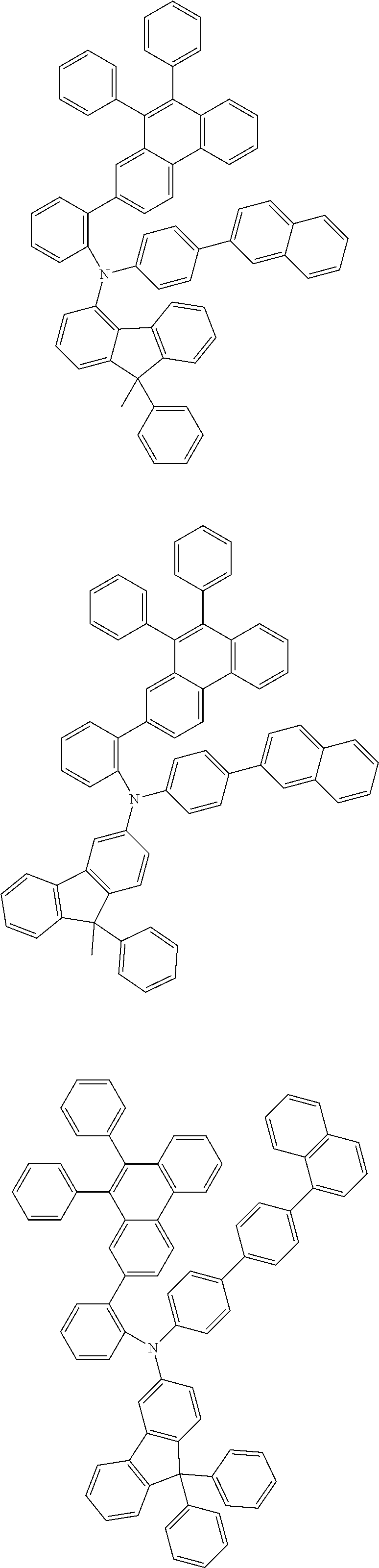 [SMILES: CC1(c2ccccc2)c2ccccc2-c2c(N(c3ccc(-c4ccc5ccccc5c4)cc3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4ccc5ccccc5c4)cc3)c3ccccc3-c3ccc4c(c3)c(-c3ccccc3)c(-c3ccccc3)c3ccccc34)ccc21 ; c1ccc(-c2c(-c3ccccc3)c3cc(-c4ccccc4N(c4ccc(-c5ccc(-c6cccc7ccccc67)cc5)cc4)c4ccc5c(c4)-c4ccccc4C5(c4ccccc4)c4ccccc4)ccc3c3ccccc23)cc1]

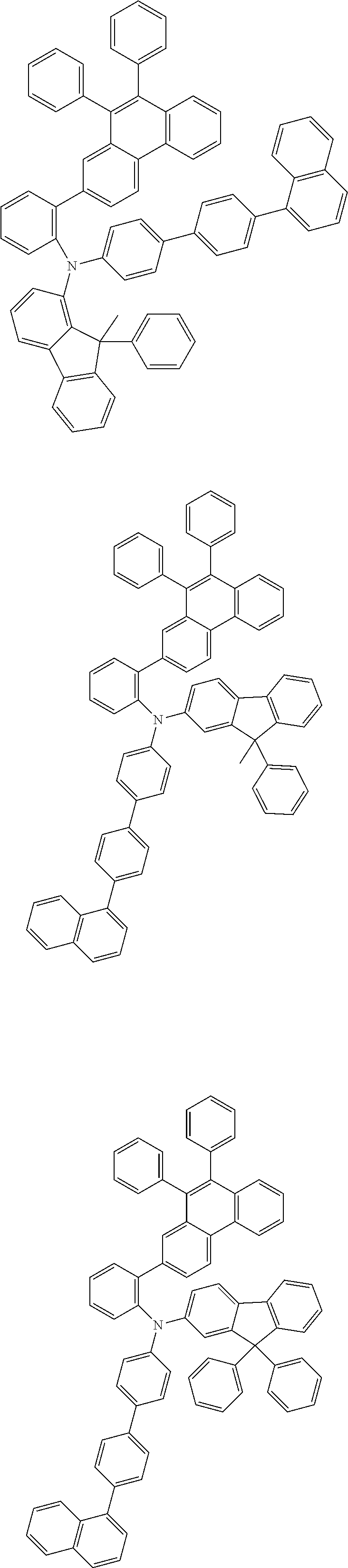 [SMILES: CC1(c2ccccc2)c2ccccc2-c2cccc(N(c3ccc(-c4ccc(-c5cccc6ccccc56)cc4)cc3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4ccc(-c5cccc6ccccc56)cc4)cc3)c3ccccc3-c3ccc4c(c3)c(-c3ccccc3)c(-c3ccccc3)c3ccccc34)cc21 ; c1ccc(-c2c(-c3ccccc3)c3cc(-c4ccccc4N(c4ccc(-c5ccc(-c6cccc7ccccc67)cc5)cc4)c4ccc5c(c4)C(c4ccccc4)(c4ccccc4)c4ccccc4-5)ccc3c3ccccc23)cc1]

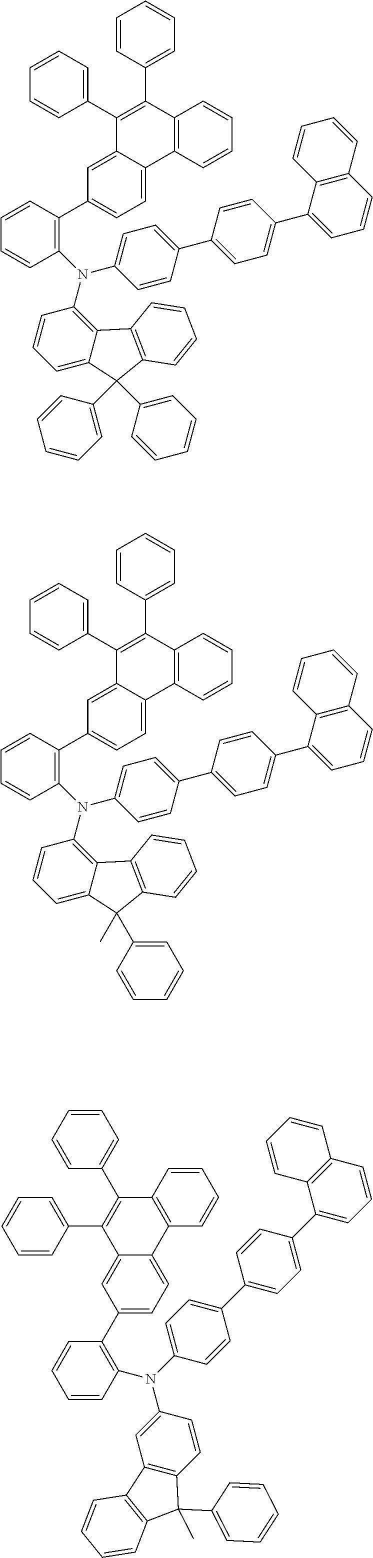 [SMILES: c1ccc(-c2c(-c3ccccc3)c3cc(-c4ccccc4N(c4ccc(-c5ccc(-c6cccc7ccccc67)cc5)cc4)c4cccc5c4-c4ccccc4C5(c4ccccc4)c4ccccc4)ccc3c3ccccc23)cc1 ; CC1(c2ccccc2)c2ccccc2-c2c(N(c3ccc(-c4ccc(-c5cccc6ccccc56)cc4)cc3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4ccc(-c5cccc6ccccc56)cc4)cc3)c3ccccc3-c3ccc4c(c3)c(-c3ccccc3)c(-c3ccccc3)c3ccccc34)ccc21]

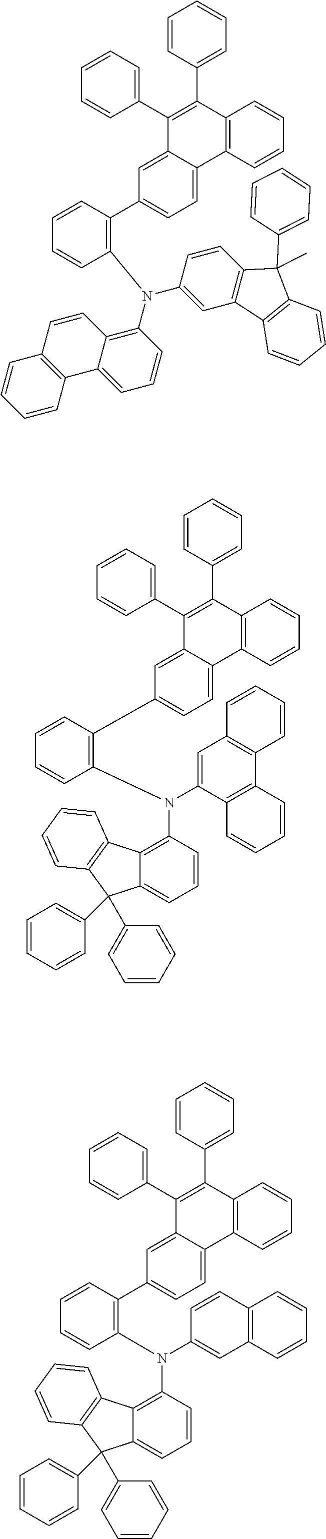 [SMILES: CC1(c2ccccc2)c2ccccc2-c2cc(N(c3ccccc3-c3ccc4c(c3)c(-c3ccccc3)c(-c3ccccc3)c3ccccc34)c3cccc4c3ccc3ccccc34)ccc21 ; c1ccc(-c2c(-c3ccccc3)c3cc(-c4ccccc4N(c4cccc5c4-c4ccccc4C5(c4ccccc4)c4ccccc4)c4cc5ccccc5c5ccccc45)ccc3c3ccccc23)cc1 ; c1ccc(-c2c(-c3ccccc3)c3cc(-c4ccccc4N(c4ccc5ccccc5c4)c4cccc5c4-c4ccccc4C5(c4ccccc4)c4ccccc4)ccc3c3ccccc23)cc1]

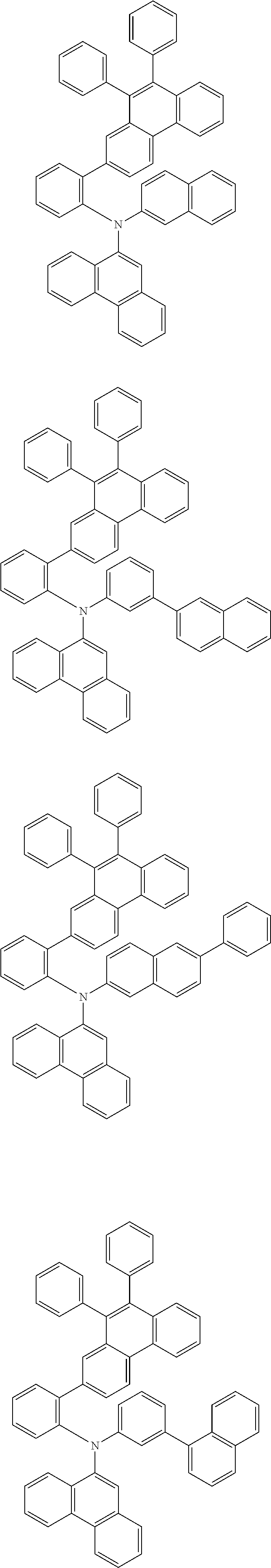 [SMILES: c1ccc(-c2c(-c3ccccc3)c3cc(-c4ccccc4N(c4ccc5ccccc5c4)c4cc5ccccc5c5ccccc45)ccc3c3ccccc23)cc1 ; c1ccc(-c2c(-c3ccccc3)c3cc(-c4ccccc4N(c4cccc(-c5ccc6ccccc6c5)c4)c4cc5ccccc5c5ccccc45)ccc3c3ccccc23)cc1 ; c1ccc(-c2ccc3cc(N(c4ccccc4-c4ccc5c(c4)c(-c4ccccc4)c(-c4ccccc4)c4ccccc45)c4cc5ccccc5c5ccccc45)ccc3c2)cc1 ; c1ccc(-c2c(-c3ccccc3)c3cc(-c4ccccc4N(c4cccc(-c5cccc6ccccc56)c4)c4cc5ccccc5c5ccccc45)ccc3c3ccccc23)cc1]

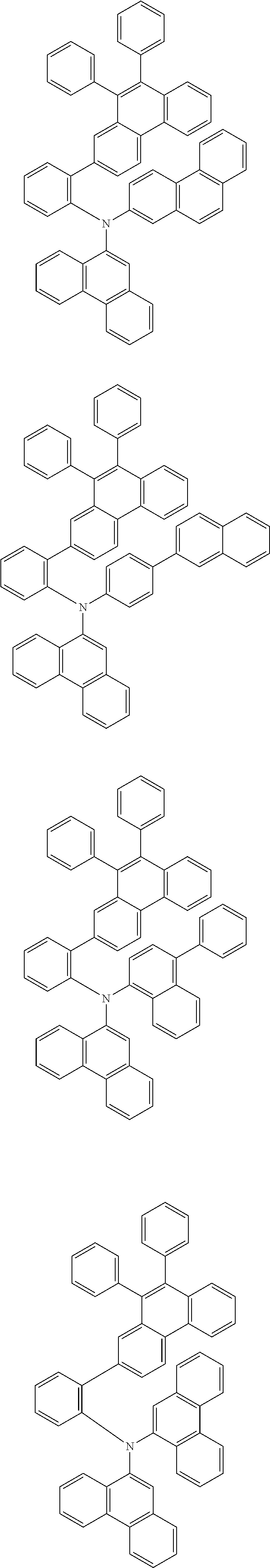 [SMILES: c1ccc(-c2c(-c3ccccc3)c3cc(-c4ccccc4N(c4ccc5c(ccc6ccccc65)c4)c4cc5ccccc5c5ccccc45)ccc3c3ccccc23)cc1 ; c1ccc(-c2c(-c3ccccc3)c3cc(-c4ccccc4N(c4ccc(-c5ccc6ccccc6c5)cc4)c4cc5ccccc5c5ccccc45)ccc3c3ccccc23)cc1 ; c1ccc(-c2ccc(N(c3ccccc3-c3ccc4c(c3)c(-c3ccccc3)c(-c3ccccc3)c3ccccc34)c3cc4ccccc4c4ccccc34)c3ccccc23)cc1 ; c1ccc(-c2c(-c3ccccc3)c3cc(-c4ccccc4N(c4cc5ccccc5c5ccccc45)c4cc5ccccc5c5ccccc45)ccc3c3ccccc23)cc1]

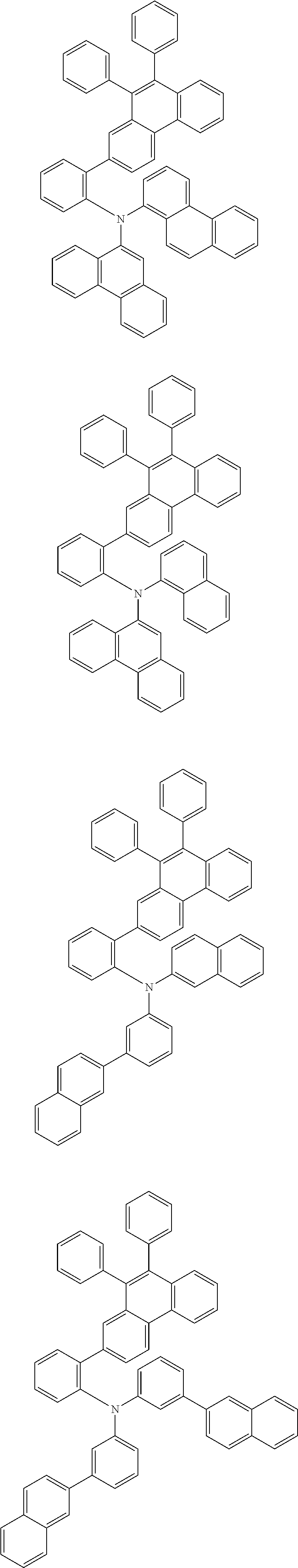 [SMILES: c1ccc(-c2c(-c3ccccc3)c3cc(-c4ccccc4N(c4cccc5c4ccc4ccccc45)c4cc5ccccc5c5ccccc45)ccc3c3ccccc23)cc1 ; c1ccc(-c2c(-c3ccccc3)c3cc(-c4ccccc4N(c4cccc5ccccc45)c4cc5ccccc5c5ccccc45)ccc3c3ccccc23)cc1 ; c1ccc(-c2c(-c3ccccc3)c3cc(-c4ccccc4N(c4cccc(-c5ccc6ccccc6c5)c4)c4ccc5ccccc5c4)ccc3c3ccccc23)cc1 ; c1ccc(-c2c(-c3ccccc3)c3cc(-c4ccccc4N(c4cccc(-c5ccc6ccccc6c5)c4)c4cccc(-c5ccc6ccccc6c5)c4)ccc3c3ccccc23)cc1]

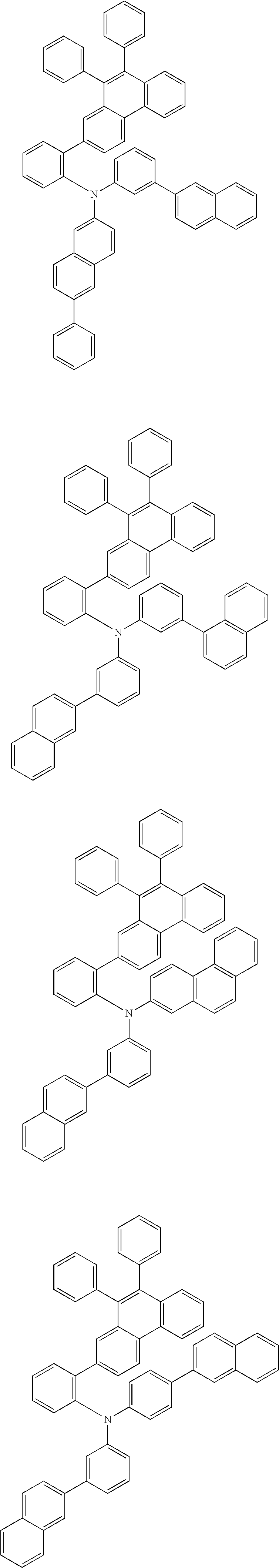 [SMILES: c1ccc(-c2ccc3cc(N(c4cccc(-c5ccc6ccccc6c5)c4)c4ccccc4-c4ccc5c(c4)c(-c4ccccc4)c(-c4ccccc4)c4ccccc45)ccc3c2)cc1 ; c1ccc(-c2c(-c3ccccc3)c3cc(-c4ccccc4N(c4cccc(-c5ccc6ccccc6c5)c4)c4cccc(-c5cccc6ccccc56)c4)ccc3c3ccccc23)cc1 ; c1ccc(-c2c(-c3ccccc3)c3cc(-c4ccccc4N(c4cccc(-c5ccc6ccccc6c5)c4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4cccc(-c5ccc6ccccc6c5)c4)ccc3c3ccccc23)cc1]

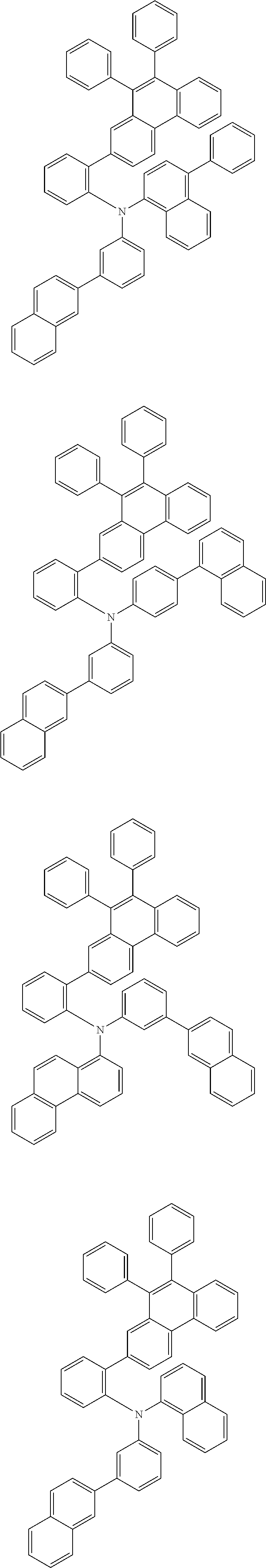 [SMILES: c1ccc(-c2ccc(N(c3cccc(-c4ccc5ccccc5c4)c3)c3ccccc3-c3ccc4c(c3)c(-c3ccccc3)c(-c3ccccc3)c3ccccc34)c3ccccc23)cc1 ; c1ccc(-c2c(-c3ccccc3)c3cc(-c4ccccc4N(c4ccc(-c5cccc6ccccc56)cc4)c4cccc(-c5ccc6ccccc6c5)c4)ccc3c3ccccc23)cc1 ; c1ccc(-c2c(-c3ccccc3)c3cc(-c4ccccc4N(c4cccc(-c5ccc6ccccc6c5)c4)c4cccc5c4ccc4ccccc45)ccc3c3ccccc23)cc1 ; c1ccc(-c2c(-c3ccccc3)c3cc(-c4ccccc4N(c4cccc(-c5ccc6ccccc6c5)c4)c4cccc5ccccc45)ccc3c3ccccc23)cc1]

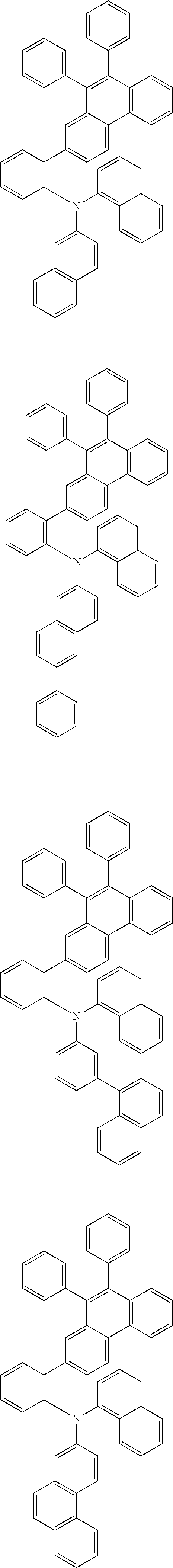 [SMILES: c1ccc(-c2c(-c3ccccc3)c3cc(-c4ccccc4N(c4ccc5ccccc5c4)c4cccc5ccccc45)ccc3c3ccccc23)cc1 ; c1ccc(-c2ccc3cc(N(c4ccccc4-c4ccc5c(c4)c(-c4ccccc4)c(-c4ccccc4)c4ccccc45)c4cccc5ccccc45)ccc3c2)cc1 ; c1ccc(-c2c(-c3ccccc3)c3cc(-c4ccccc4N(c4cccc(-c5cccc6ccccc56)c4)c4cccc5ccccc45)ccc3c3ccccc23)cc1 ; c1ccc(-c2c(-c3ccccc3)c3cc(-c4ccccc4N(c4ccc5c(ccc6ccccc65)c4)c4cccc5ccccc45)ccc3c3ccccc23)cc1]

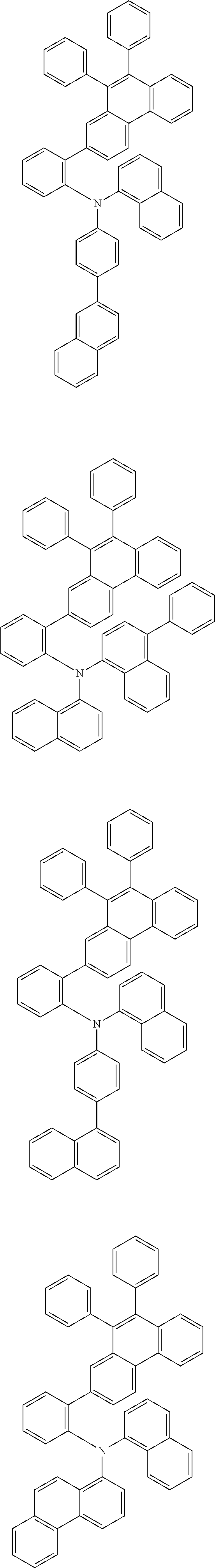 [SMILES: c1ccc(-c2c(-c3ccccc3)c3cc(-c4ccccc4N(c4ccc(-c5ccc6ccccc6c5)cc4)c4cccc5ccccc45)ccc3c3ccccc23)cc1 ; c1ccc(-c2ccc(N(c3ccccc3-c3ccc4c(c3)c(-c3ccccc3)c(-c3ccccc3)c3ccccc34)c3cccc4ccccc34)c3ccccc23)cc1 ; c1ccc(-c2c(-c3ccccc3)c3cc(-c4ccccc4N(c4ccc(-c5cccc6ccccc56)cc4)c4cccc5ccccc45)ccc3c3ccccc23)cc1 ; c1ccc(-c2c(-c3ccccc3)c3cc(-c4ccccc4N(c4cccc5ccccc45)c4cccc5c4ccc4ccccc45)ccc3c3ccccc23)cc1]

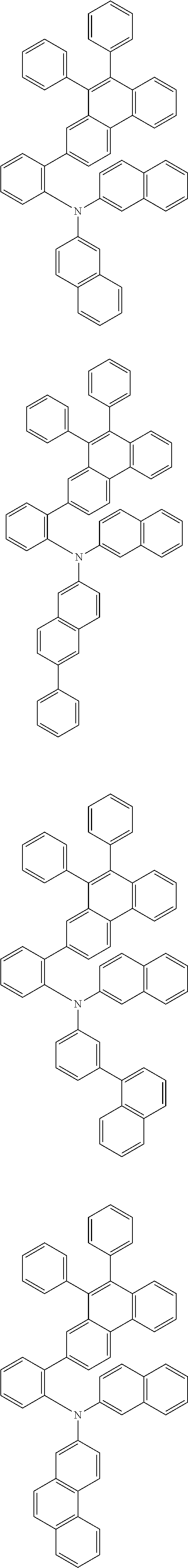

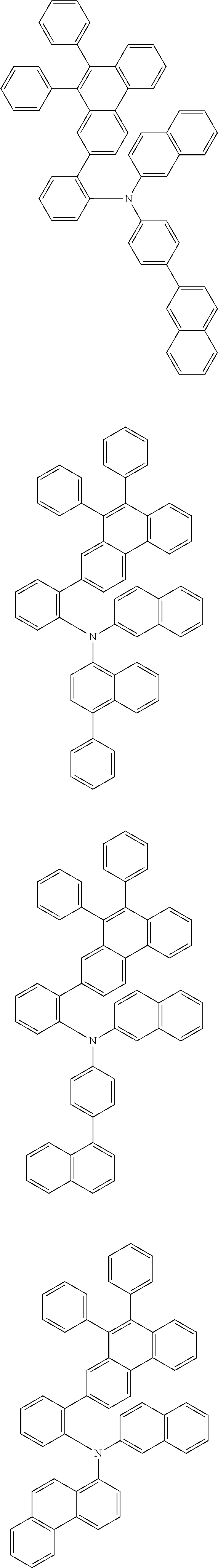 [SMILES: c1ccc(-c2c(-c3ccccc3)c3cc(-c4ccccc4N(c4ccc(-c5ccc6ccccc6c5)cc4)c4ccc5ccccc5c4)ccc3c3ccccc23)cc1 ; c1ccc(-c2ccc(N(c3ccc4ccccc4c3)c3ccccc3-c3ccc4c(c3)c(-c3ccccc3)c(-c3ccccc3)c3ccccc34)c3ccccc23)cc1 ; c1ccc(-c2c(-c3ccccc3)c3cc(-c4ccccc4N(c4ccc(-c5cccc6ccccc56)cc4)c4ccc5ccccc5c4)ccc3c3ccccc23)cc1 ; c1ccc(-c2c(-c3ccccc3)c3cc(-c4ccccc4N(c4ccc5ccccc5c4)c4cccc5c4ccc4ccccc45)ccc3c3ccccc23)cc1]

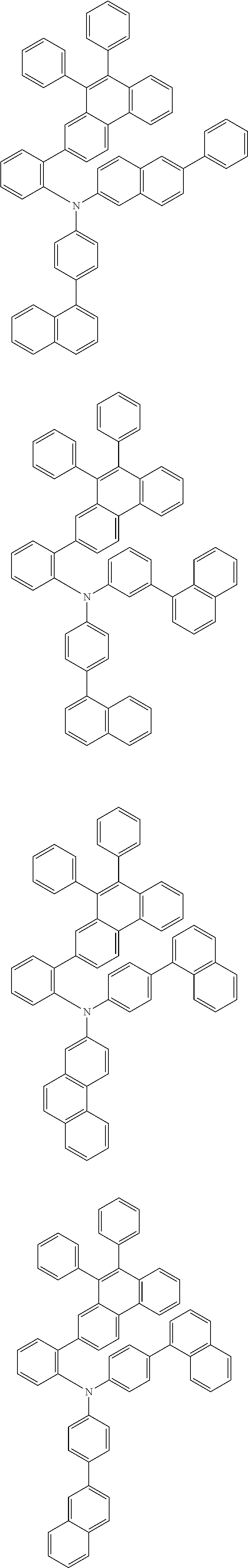 [SMILES: c1ccc(-c2ccc3cc(N(c4ccc(-c5cccc6ccccc56)cc4)c4ccccc4-c4ccc5c(c4)c(-c4ccccc4)c(-c4ccccc4)c4ccccc45)ccc3c2)cc1 ; c1ccc(-c2c(-c3ccccc3)c3cc(-c4ccccc4N(c4ccc(-c5cccc6ccccc56)cc4)c4cccc(-c5cccc6ccccc56)c4)ccc3c3ccccc23)cc1 ; c1ccc(-c2c(-c3ccccc3)c3cc(-c4ccccc4N(c4ccc(-c5cccc6ccccc56)cc4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4ccc(-c5cccc6ccccc56)cc4)ccc3c3ccccc23)cc1]

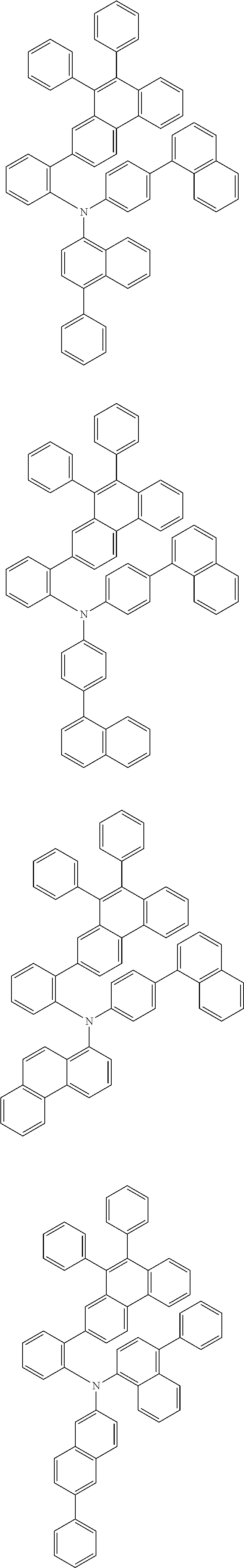 [SMILES: c1ccc(-c2ccc(N(c3ccc(-c4cccc5ccccc45)cc3)c3ccccc3-c3ccc4c(c3)c(-c3ccccc3)c(-c3ccccc3)c3ccccc34)c3ccccc23)cc1 ; c1ccc(-c2c(-c3ccccc3)c3cc(-c4ccccc4N(c4ccc(-c5cccc6ccccc56)cc4)c4ccc(-c5cccc6ccccc56)cc4)ccc3c3ccccc23)cc1 ; c1ccc(-c2c(-c3ccccc3)c3cc(-c4ccccc4N(c4ccc(-c5cccc6ccccc56)cc4)c4cccc5c4ccc4ccccc45)ccc3c3ccccc23)cc1 ; c1ccc(-c2ccc3cc(N(c4ccccc4-c4ccc5c(c4)c(-c4ccccc4)c(-c4ccccc4)c4ccccc45)c4ccc(-c5ccccc5)c5ccccc45)ccc3c2)cc1]

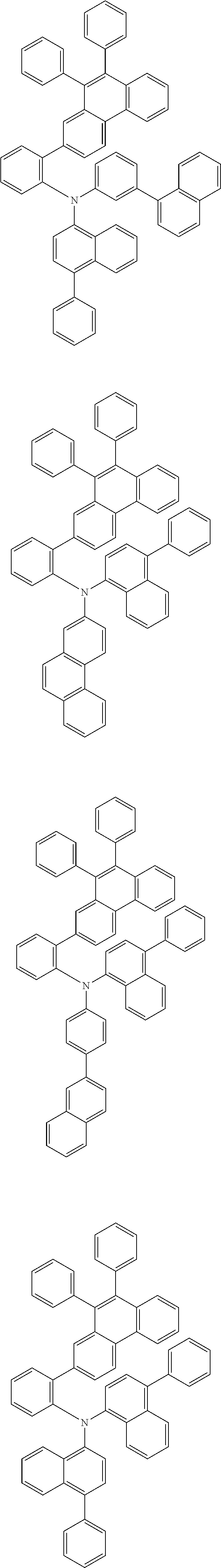 [SMILES: c1ccc(-c2ccc(N(c3cccc(-c4cccc5ccccc45)c3)c3ccccc3-c3ccc4c(c3)c(-c3ccccc3)c(-c3ccccc3)c3ccccc34)c3ccccc23)cc1 ; c1ccc(-c2ccc(N(c3ccc4c(ccc5ccccc54)c3)c3ccccc3-c3ccc4c(c3)c(-c3ccccc3)c(-c3ccccc3)c3ccccc34)c3ccccc23)cc1 ; c1ccc(-c2ccc(N(c3ccc(-c4ccc5ccccc5c4)cc3)c3ccccc3-c3ccc4c(c3)c(-c3ccccc3)c(-c3ccccc3)c3ccccc34)c3ccccc23)cc1 ; c1ccc(-c2ccc(N(c3ccccc3-c3ccc4c(c3)c(-c3ccccc3)c(-c3ccccc3)c3ccccc34)c3ccc(-c4ccccc4)c4ccccc34)c3ccccc23)cc1]

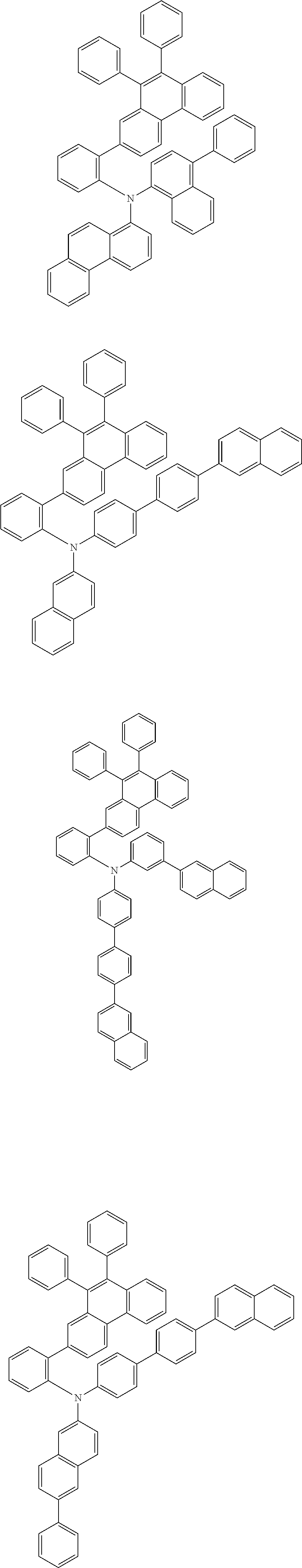 [SMILES: c1ccc(-c2ccc(N(c3ccccc3-c3ccc4c(c3)c(-c3ccccc3)c(-c3ccccc3)c3ccccc34)c3cccc4c3ccc3ccccc34)c3ccccc23)cc1 ; c1ccc(-c2c(-c3ccccc3)c3cc(-c4ccccc4N(c4ccc(-c5ccc(-c6ccc7ccccc7c6)cc5)cc4)c4ccc5ccccc5c4)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc(-c5ccc6ccccc6c5)c4)ccc3c3ccccc23)cc1 ; c1ccc(-c2ccc3cc(N(c4ccc(-c5ccc(-c6ccc7ccccc7c6)cc5)cc4)c4ccccc4-c4ccc5c(c4)c(-c4ccccc4)c(-c4ccccc4)c4ccccc45)ccc3c2)cc1]

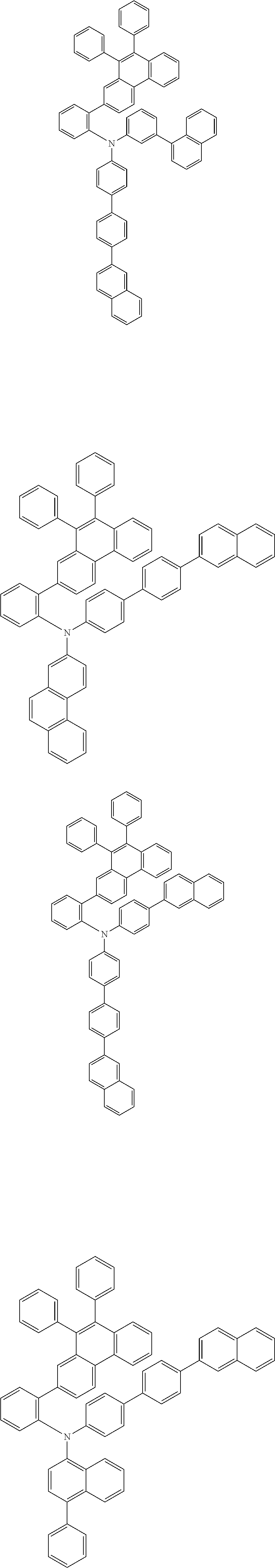 [SMILES: c1ccc(-c2c(-c3ccccc3)c3cc(-c4ccccc4N(c4ccc(-c5ccc(-c6ccc7ccccc7c6)cc5)cc4)c4cccc(-c5cccc6ccccc56)c4)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4ccc5c(ccc6ccccc65)c4)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4ccc(-c5ccc6ccccc6c5)cc4)ccc3c3ccccc23)cc1 ; c1ccc(-c2ccc(N(c3ccc(-c4ccc(-c5ccc6ccccc6c5)cc4)cc3)c3ccccc3-c3ccc4c(c3)c(-c3ccccc3)c(-c3ccccc3)c3ccccc34)c3ccccc23)cc1]

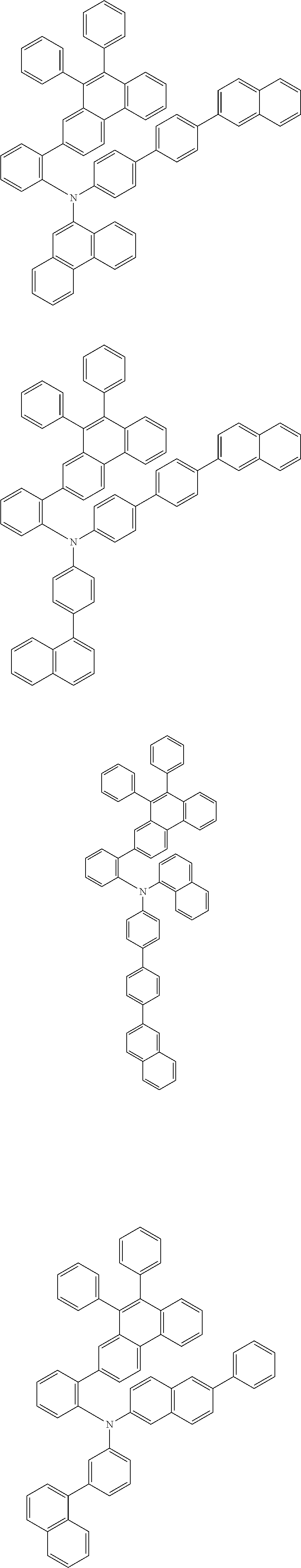 [SMILES: c1ccc(-c2c(-c3ccccc3)c3cc(-c4ccccc4N(c4ccc(-c5ccc(-c6ccc7ccccc7c6)cc5)cc4)c4cc5ccccc5c5ccccc45)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4ccc(-c5cccc6ccccc56)cc4)ccc3c3ccccc23)cc1 ; c1ccc(-c2c(-c3ccccc3)c3cc(-c4ccccc4N(c4ccc(-c5ccc(-c6ccc7ccccc7c6)cc5)cc4)c4cccc5ccccc45)ccc3c3ccccc23)cc1 ; c1ccc(-c2ccc3cc(N(c4cccc(-c5cccc6ccccc56)c4)c4ccccc4-c4ccc5c(c4)c(-c4ccccc4)c(-c4ccccc4)c4ccccc45)ccc3c2)cc1]

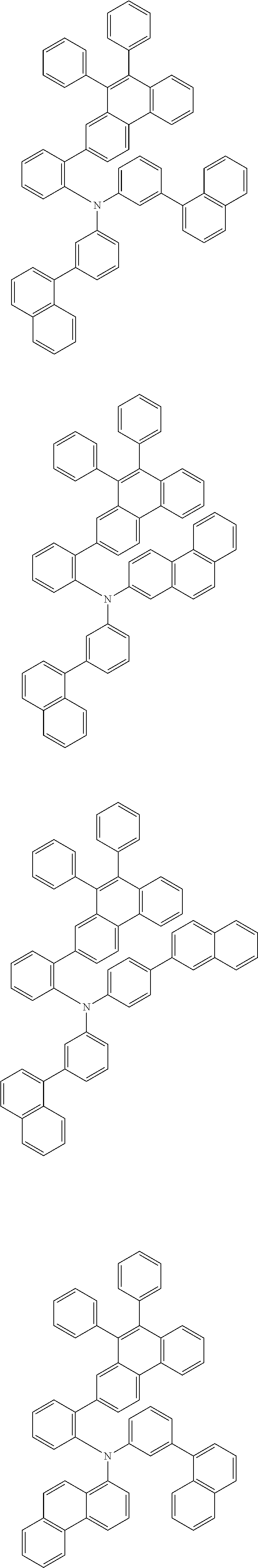 [SMILES: c1ccc(-c2c(-c3ccccc3)c3cc(-c4ccccc4N(c4cccc(-c5cccc6ccccc56)c4)c4cccc(-c5cccc6ccccc56)c4)ccc3c3ccccc23)cc1 ; c1ccc(-c2c(-c3ccccc3)c3cc(-c4ccccc4N(c4cccc(-c5cccc6ccccc56)c4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4cccc(-c5cccc6ccccc56)c4)ccc3c3ccccc23)cc1 ; c1ccc(-c2c(-c3ccccc3)c3cc(-c4ccccc4N(c4cccc(-c5cccc6ccccc56)c4)c4cccc5c4ccc4ccccc45)ccc3c3ccccc23)cc1]

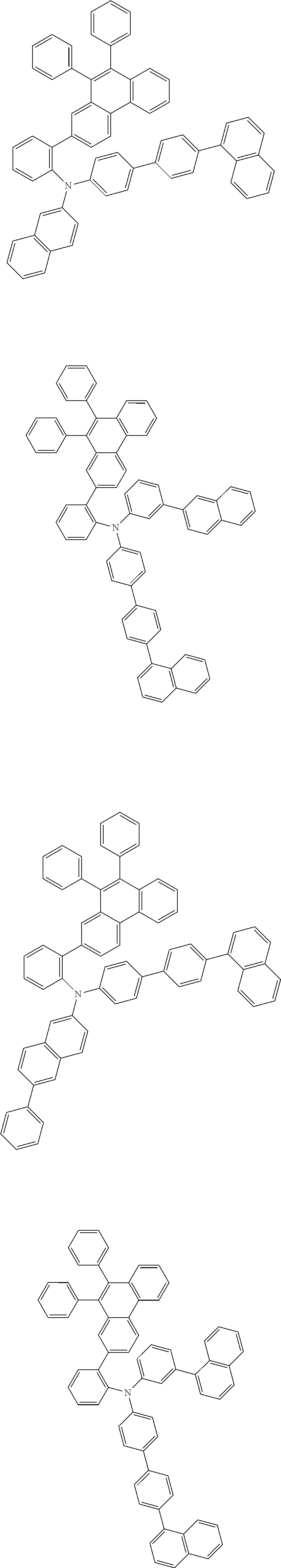 [SMILES: c1ccc(-c2c(-c3ccccc3)c3cc(-c4ccccc4N(c4ccc(-c5ccc(-c6cccc7ccccc67)cc5)cc4)c4ccc5ccccc5c4)ccc3c3ccccc23)cc1 ; c1ccc(-c2c(-c3ccccc3)c3cc(-c4ccccc4N(c4ccc(-c5ccc(-c6cccc7ccccc67)cc5)cc4)c4cccc(-c5ccc6ccccc6c5)c4)ccc3c3ccccc23)cc1 ; c1ccc(-c2ccc3cc(N(c4ccc(-c5ccc(-c6cccc7ccccc67)cc5)cc4)c4ccccc4-c4ccc5c(c4)c(-c4ccccc4)c(-c4ccccc4)c4ccccc45)ccc3c2)cc1 ; c1ccc(-c2c(-c3ccccc3)c3cc(-c4ccccc4N(c4ccc(-c5ccc(-c6cccc7ccccc67)cc5)cc4)c4cccc(-c5cccc6ccccc56)c4)ccc3c3ccccc23)cc1]

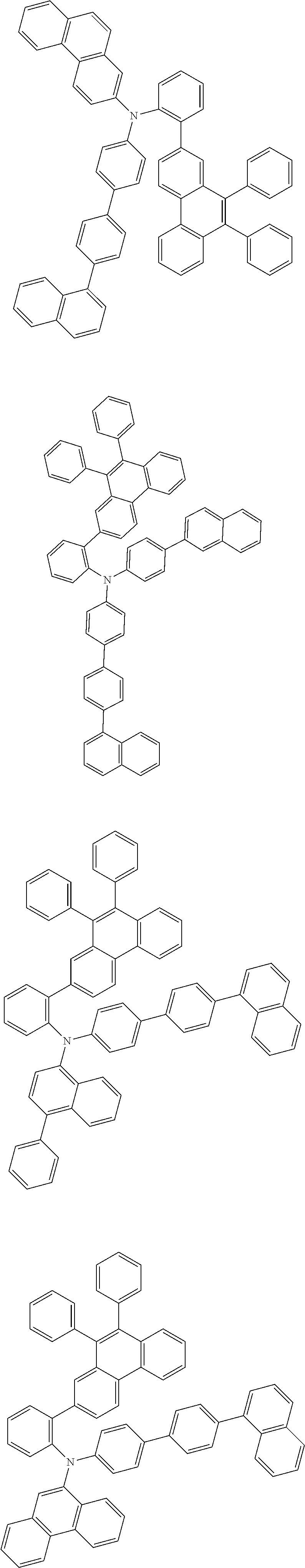 [SMILES: c1ccc(-c2c(-c3ccccc3)c3cc(-c4ccccc4N(c4ccc(-c5ccc(-c6cccc7ccccc67)cc5)cc4)c4ccc5c(ccc6ccccc65)c4)ccc3c3ccccc23)cc1 ; c1ccc(-c2c(-c3ccccc3)c3cc(-c4ccccc4N(c4ccc(-c5ccc(-c6cccc7ccccc67)cc5)cc4)c4ccc(-c5ccc6ccccc6c5)cc4)ccc3c3ccccc23)cc1 ; c1ccc(-c2ccc(N(c3ccc(-c4ccc(-c5cccc6ccccc56)cc4)cc3)c3ccccc3-c3ccc4c(c3)c(-c3ccccc3)c(-c3ccccc3)c3ccccc34)c3ccccc23)cc1 ; c1ccc(-c2c(-c3ccccc3)c3cc(-c4ccccc4N(c4ccc(-c5ccc(-c6cccc7ccccc67)cc5)cc4)c4cc5ccccc5c5ccccc45)ccc3c3ccccc23)cc1]

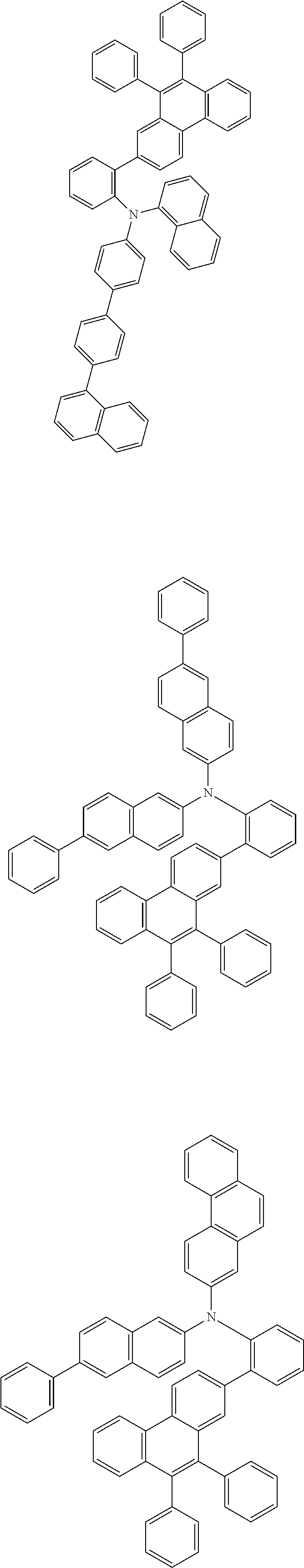 [SMILES: c1ccc(-c2c(-c3ccccc3)c3cc(-c4ccccc4N(c4ccc(-c5ccc(-c6cccc7ccccc67)cc5)cc4)c4cccc5ccccc45)ccc3c3ccccc23)cc1 ; c1ccc(-c2ccc3cc(N(c4ccc5cc(-c6ccccc6)ccc5c4)c4ccccc4-c4ccc5c(c4)c(-c4ccccc4)c(-c4ccccc4)c4ccccc45)ccc3c2)cc1 ; c1ccc(-c2ccc3cc(N(c4ccc5c(ccc6ccccc65)c4)c4ccccc4-c4ccc5c(c4)c(-c4ccccc4)c(-c4ccccc4)c4ccccc45)ccc3c2)cc1]

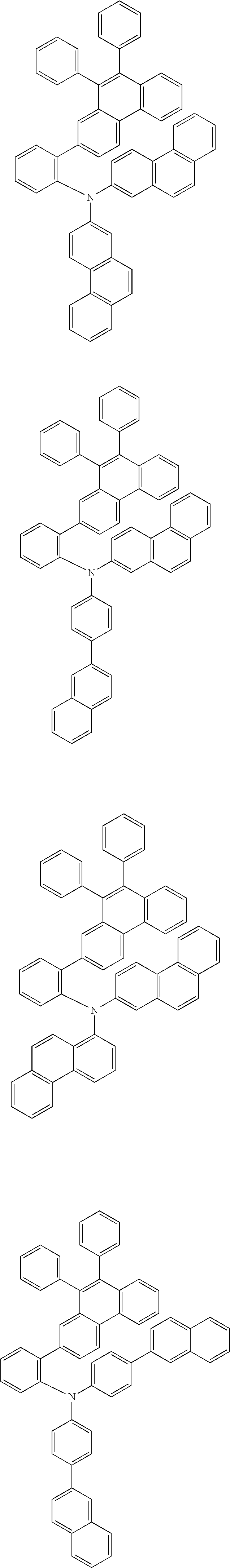 [SMILES: c1ccc(-c2c(-c3ccccc3)c3cc(-c4ccccc4N(c4ccc5c(ccc6ccccc65)c4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4ccc5c(ccc6ccccc65)c4)ccc3c3ccccc23)cc1 ; c1ccc(-c2c(-c3ccccc3)c3cc(-c4ccccc4N(c4ccc5c(ccc6ccccc65)c4)c4cccc5c4ccc4ccccc45)ccc3c3ccccc23)cc1 ; c1ccc(-c2c(-c3ccccc3)c3cc(-c4ccccc4N(c4ccc(-c5ccc6ccccc6c5)cc4)c4ccc(-c5ccc6ccccc6c5)cc4)ccc3c3ccccc23)cc1]

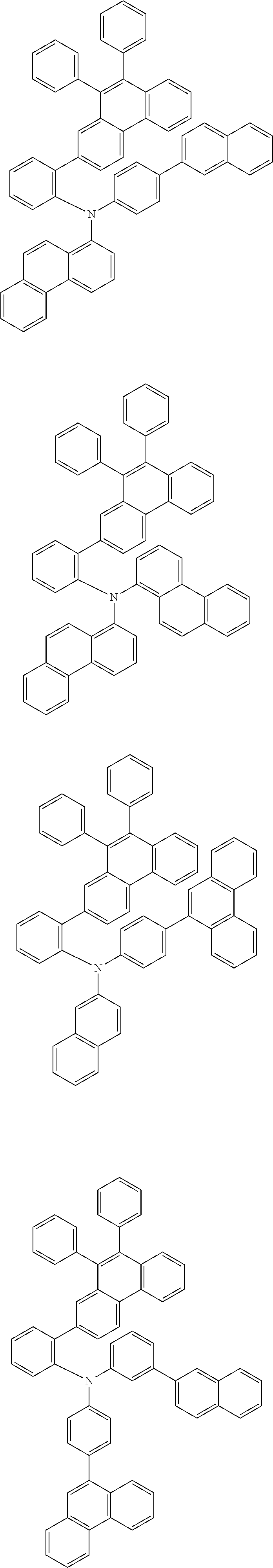 [SMILES: c1ccc(-c2c(-c3ccccc3)c3cc(-c4ccccc4N(c4ccc(-c5ccc6ccccc6c5)cc4)c4cccc5c4ccc4ccccc45)ccc3c3ccccc23)cc1 ; c1ccc(-c2c(-c3ccccc3)c3cc(-c4ccccc4N(c4cccc5c4ccc4ccccc45)c4cccc5c4ccc4ccccc45)ccc3c3ccccc23)cc1 ; c1ccc(-c2c(-c3ccccc3)c3cc(-c4ccccc4N(c4ccc(-c5cc6ccccc6c6ccccc56)cc4)c4ccc5ccccc5c4)ccc3c3ccccc23)cc1 ; c1ccc(-c2c(-c3ccccc3)c3cc(-c4ccccc4N(c4ccc(-c5cc6ccccc6c6ccccc56)cc4)c4cccc(-c5ccc6ccccc6c5)c4)ccc3c3ccccc23)cc1]

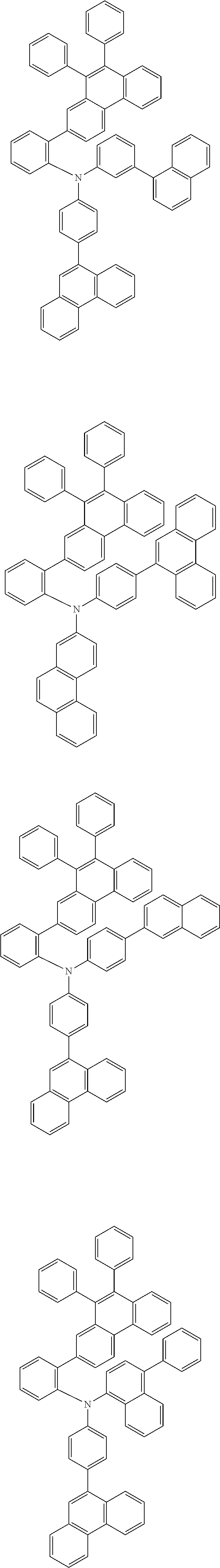 [SMILES: c1ccc(-c2c(-c3ccccc3)c3cc(-c4ccccc4N(c4ccc(-c5cc6ccccc6c6ccccc56)cc4)c4cccc(-c5cccc6ccccc56)c4)ccc3c3ccccc23)cc1 ; c1ccc(-c2c(-c3ccccc3)c3cc(-c4ccccc4N(c4ccc(-c5cc6ccccc6c6ccccc56)cc4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4ccc(-c5cc6ccccc6c6ccccc56)cc4)ccc3c3ccccc23)cc1 ; c1ccc(-c2ccc(N(c3ccc(-c4cc5ccccc5c5ccccc45)cc3)c3ccccc3-c3ccc4c(c3)c(-c3ccccc3)c(-c3ccccc3)c3ccccc34)c3ccccc23)cc1]

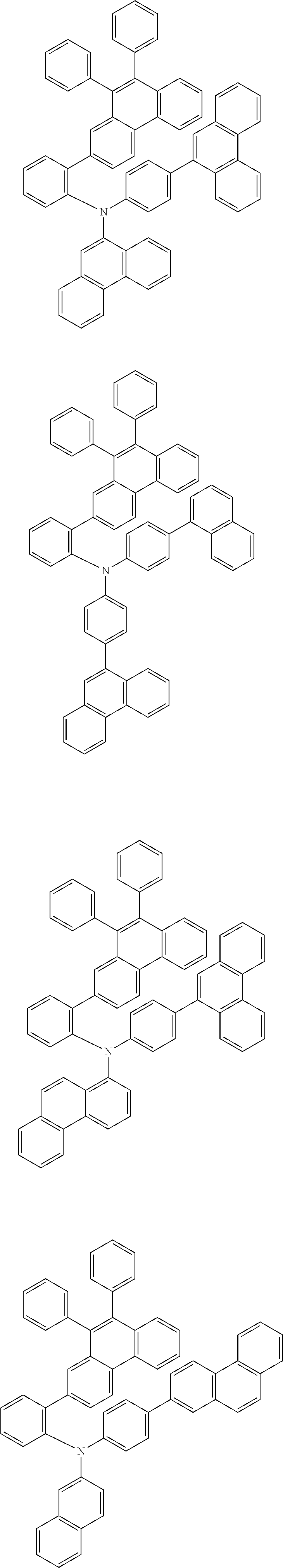 [SMILES: c1ccc(-c2c(-c3ccccc3)c3cc(-c4ccccc4N(c4ccc(-c5cc6ccccc6c6ccccc56)cc4)c4cc5ccccc5c5ccccc45)ccc3c3ccccc23)cc1 ; c1ccc(-c2c(-c3ccccc3)c3cc(-c4ccccc4N(c4ccc(-c5cccc6ccccc56)cc4)c4ccc(-c5cc6ccccc6c6ccccc56)cc4)ccc3c3ccccc23)cc1 ; c1ccc(-c2c(-c3ccccc3)c3cc(-c4ccccc4N(c4ccc(-c5cc6ccccc6c6ccccc56)cc4)c4cccc5c4ccc4ccccc45)ccc3c3ccccc23)cc1 ; c1ccc(-c2c(-c3ccccc3)c3cc(-c4ccccc4N(c4ccc(-c5ccc6c(ccc7ccccc76)c5)cc4)c4ccc5ccccc5c4)ccc3c3ccccc23)cc1]

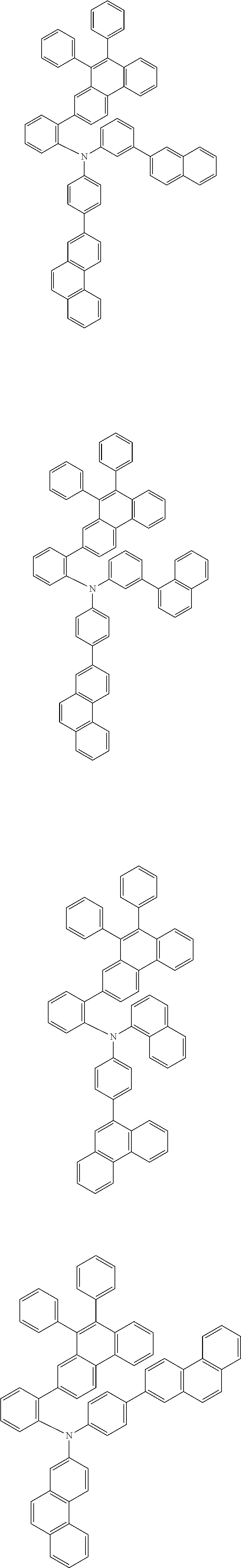 [SMILES: c1ccc(-c2c(-c3ccccc3)c3cc(-c4ccccc4N(c4ccc(-c5ccc6c(ccc7ccccc76)c5)cc4)c4cccc(-c5ccc6ccccc6c5)c4)ccc3c3ccccc23)cc1 ; c1ccc(-c2c(-c3ccccc3)c3cc(-c4ccccc4N(c4ccc(-c5ccc6c(ccc7ccccc76)c5)cc4)c4cccc(-c5cccc6ccccc56)c4)ccc3c3ccccc23)cc1 ; c1ccc(-c2c(-c3ccccc3)c3cc(-c4ccccc4N(c4ccc(-c5cc6ccccc6c6ccccc56)cc4)c4cccc5ccccc45)ccc3c3ccccc23)cc1 ; c1ccc(-c2c(-c3ccccc3)c3cc(-c4ccccc4N(c4ccc(-c5ccc6c(ccc7ccccc76)c5)cc4)c4ccc5c(ccc6ccccc65)c4)ccc3c3ccccc23)cc1]

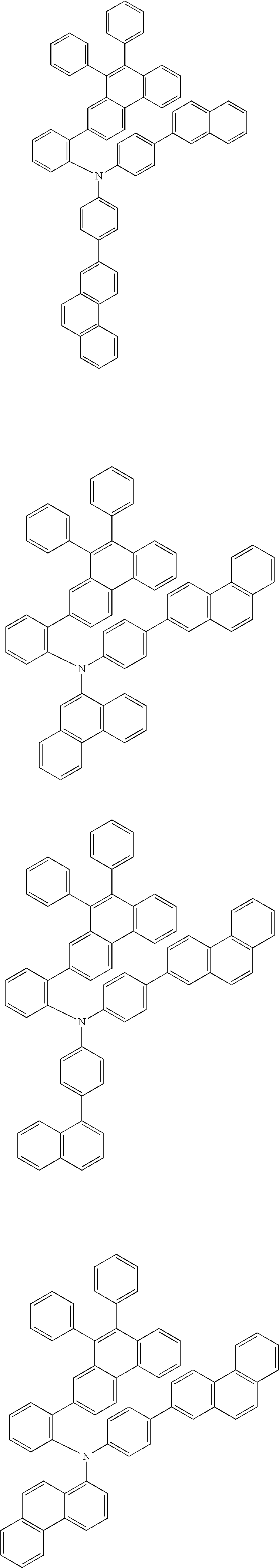 [SMILES: c1ccc(-c2c(-c3ccccc3)c3cc(-c4ccccc4N(c4ccc(-c5ccc6ccccc6c5)cc4)c4ccc(-c5ccc6c(ccc7ccccc76)c5)cc4)ccc3c3ccccc23)cc1 ; c1ccc(-c2c(-c3ccccc3)c3cc(-c4ccccc4N(c4ccc(-c5ccc6c(ccc7ccccc76)c5)cc4)c4cc5ccccc5c5ccccc45)ccc3c3ccccc23)cc1 ; c1ccc(-c2c(-c3ccccc3)c3cc(-c4ccccc4N(c4ccc(-c5ccc6c(ccc7ccccc76)c5)cc4)c4ccc(-c5cccc6ccccc56)cc4)ccc3c3ccccc23)cc1 ; c1ccc(-c2c(-c3ccccc3)c3cc(-c4ccccc4N(c4ccc(-c5ccc6c(ccc7ccccc76)c5)cc4)c4cccc5c4ccc4ccccc45)ccc3c3ccccc23)cc1]

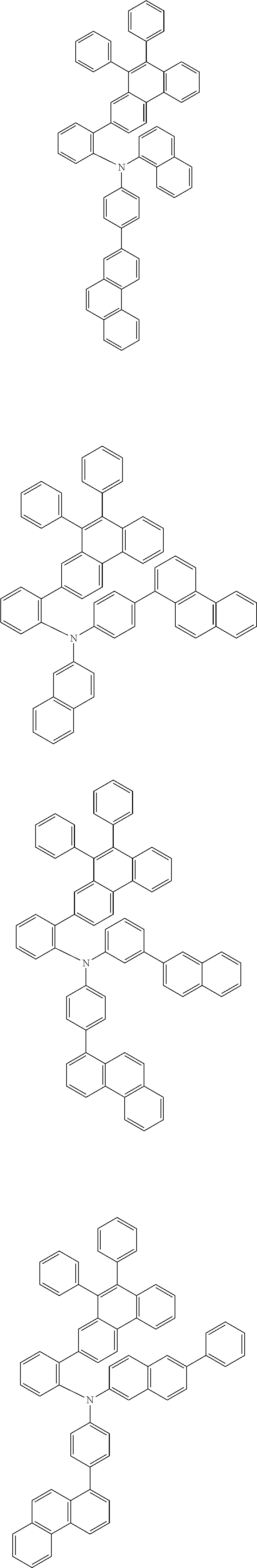 [SMILES: c1ccc(-c2c(-c3ccccc3)c3cc(-c4ccccc4N(c4ccc(-c5ccc6c(ccc7ccccc76)c5)cc4)c4cccc5ccccc45)ccc3c3ccccc23)cc1 ; c1ccc(-c2c(-c3ccccc3)c3cc(-c4ccccc4N(c4ccc(-c5cccc6c5ccc5ccccc56)cc4)c4ccc5ccccc5c4)ccc3c3ccccc23)cc1 ; c1ccc(-c2c(-c3ccccc3)c3cc(-c4ccccc4N(c4ccc(-c5cccc6c5ccc5ccccc56)cc4)c4cccc(-c5ccc6ccccc6c5)c4)ccc3c3ccccc23)cc1 ; c1ccc(-c2ccc3cc(N(c4ccc(-c5cccc6c5ccc5ccccc56)cc4)c4ccccc4-c4ccc5c(c4)c(-c4ccccc4)c(-c4ccccc4)c4ccccc45)ccc3c2)cc1]

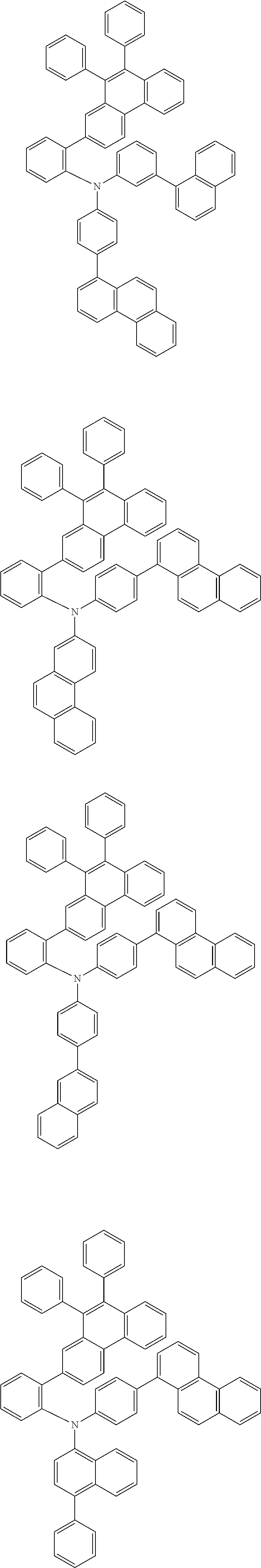 [SMILES: c1ccc(-c2c(-c3ccccc3)c3cc(-c4ccccc4N(c4ccc(-c5cccc6c5ccc5ccccc56)cc4)c4cccc(-c5cccc6ccccc56)c4)ccc3c3ccccc23)cc1 ; c1ccc(-c2c(-c3ccccc3)c3cc(-c4ccccc4N(c4ccc(-c5cccc6c5ccc5ccccc56)cc4)c4ccc5c(ccc6ccccc65)c4)ccc3c3ccccc23)cc1 ; c1ccc(-c2c(-c3ccccc3)c3cc(-c4ccccc4N(c4ccc(-c5ccc6ccccc6c5)cc4)c4ccc(-c5cccc6c5ccc5ccccc56)cc4)ccc3c3ccccc23)cc1 ; c1ccc(-c2ccc(N(c3ccc(-c4cccc5c4ccc4ccccc45)cc3)c3ccccc3-c3ccc4c(c3)c(-c3ccccc3)c(-c3ccccc3)c3ccccc34)c3ccccc23)cc1]

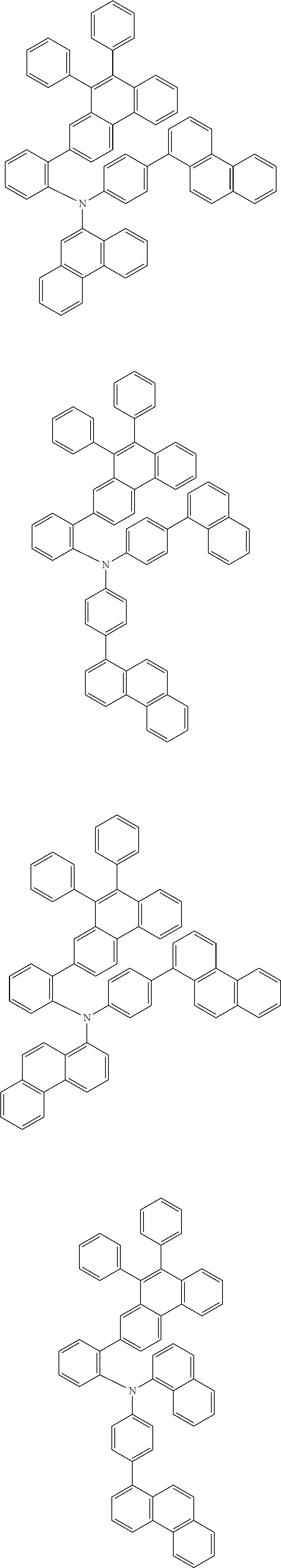 [SMILES: c1ccc(-c2c(-c3ccccc3)c3cc(-c4ccccc4N(c4ccc(-c5cccc6c5ccc5ccccc56)cc4)c4cc5ccccc5c5ccccc45)ccc3c3ccccc23)cc1 ; c1ccc(-c2c(-c3ccccc3)c3cc(-c4ccccc4N(c4ccc(-c5cccc6ccccc56)cc4)c4ccc(-c5cccc6c5ccc5ccccc56)cc4)ccc3c3ccccc23)cc1 ; c1ccc(-c2c(-c3ccccc3)c3cc(-c4ccccc4N(c4ccc(-c5cccc6c5ccc5ccccc56)cc4)c4cccc5c4ccc4ccccc45)ccc3c3ccccc23)cc1 ; c1ccc(-c2c(-c3ccccc3)c3cc(-c4ccccc4N(c4ccc(-c5cccc6c5ccc5ccccc56)cc4)c4cccc5ccccc45)ccc3c3ccccc23)cc1]

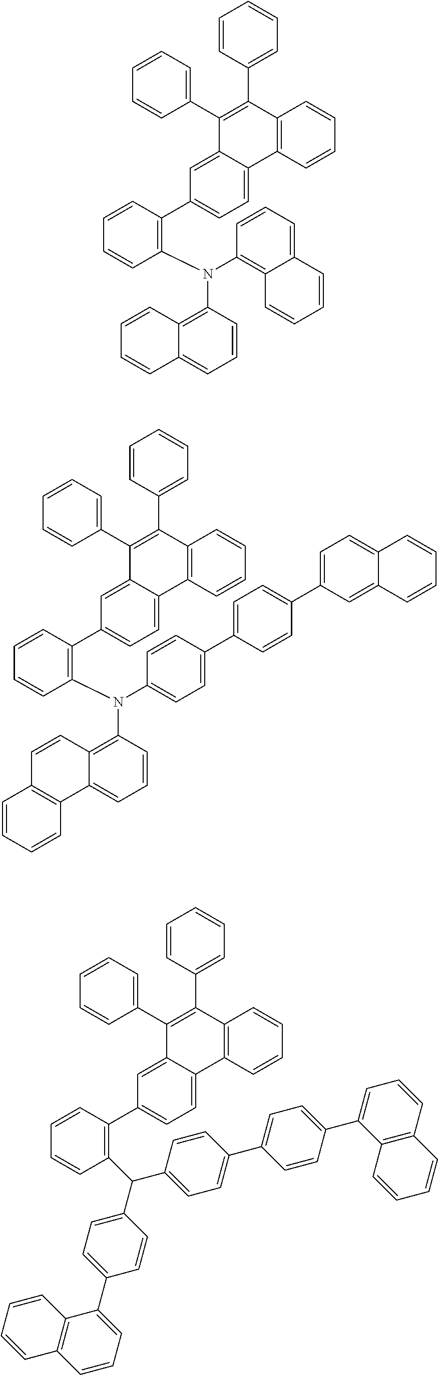

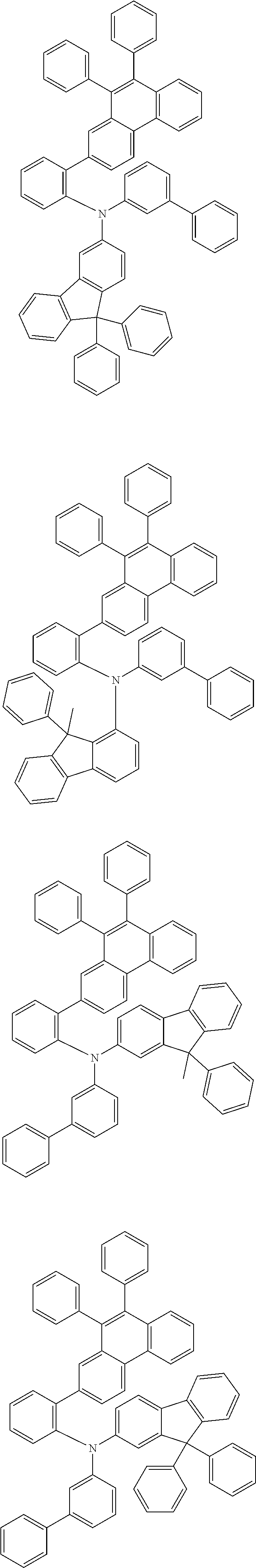 [SMILES: c1ccc(-c2cccc(N(c3ccc4c(c3)-c3ccccc3C4(c3ccccc3)c3ccccc3)c3ccccc3-c3ccc4c(c3)c(-c3ccccc3)c(-c3ccccc3)c3ccccc34)c2)cc1 ; CC1(c2ccccc2)c2ccccc2-c2cccc(N(c3cccc(-c4ccccc4)c3)c3ccccc3-c3ccc4c(c3)c(-c3ccccc3)c(-c3ccccc3)c3ccccc34)c21 ; CC1(c2ccccc2)c2ccccc2-c2ccc(N(c3cccc(-c4ccccc4)c3)c3ccccc3-c3ccc4c(c3)c(-c3ccccc3)c(-c3ccccc3)c3ccccc34)cc21 ; c1ccc(-c2cccc(N(c3ccc4c(c3)C(c3ccccc3)(c3ccccc3)c3ccccc3-4)c3ccccc3-c3ccc4c(c3)c(-c3ccccc3)c(-c3ccccc3)c3ccccc34)c2)cc1]

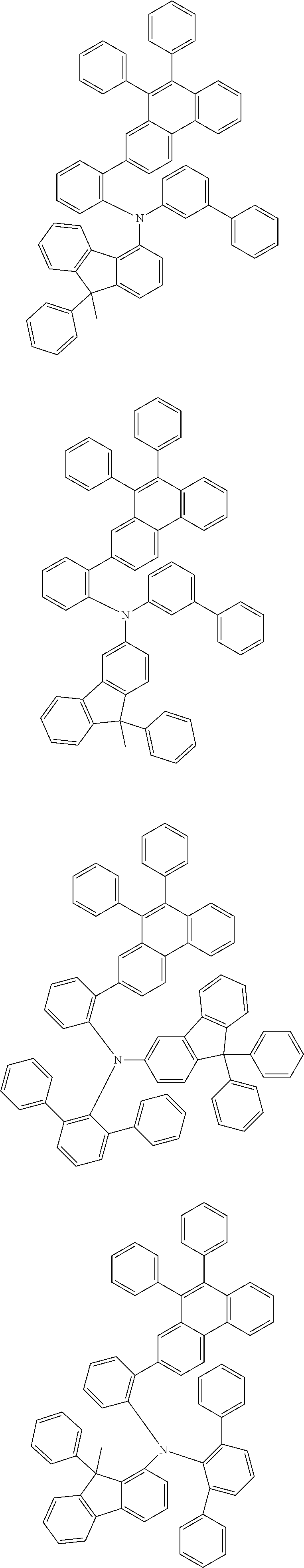 [SMILES: CC1(c2ccccc2)c2ccccc2-c2c(N(c3cccc(-c4ccccc4)c3)c3ccccc3-c3ccc4c(c3)c(-c3ccccc3)c(-c3ccccc3)c3ccccc34)cccc21 ; CC1(c2ccccc2)c2ccccc2-c2cc(N(c3cccc(-c4ccccc4)c3)c3ccccc3-c3ccc4c(c3)c(-c3ccccc3)c(-c3ccccc3)c3ccccc34)ccc21 ; c1ccc(-c2cccc(-c3ccccc3)c2N(c2ccc3c(c2)-c2ccccc2C3(c2ccccc2)c2ccccc2)c2ccccc2-c2ccc3c(c2)c(-c2ccccc2)c(-c2ccccc2)c2ccccc23)cc1 ; CC1(c2ccccc2)c2ccccc2-c2cccc(N(c3ccccc3-c3ccc4c(c3)c(-c3ccccc3)c(-c3ccccc3)c3ccccc34)c3c(-c4ccccc4)cccc3-c3ccccc3)c21]

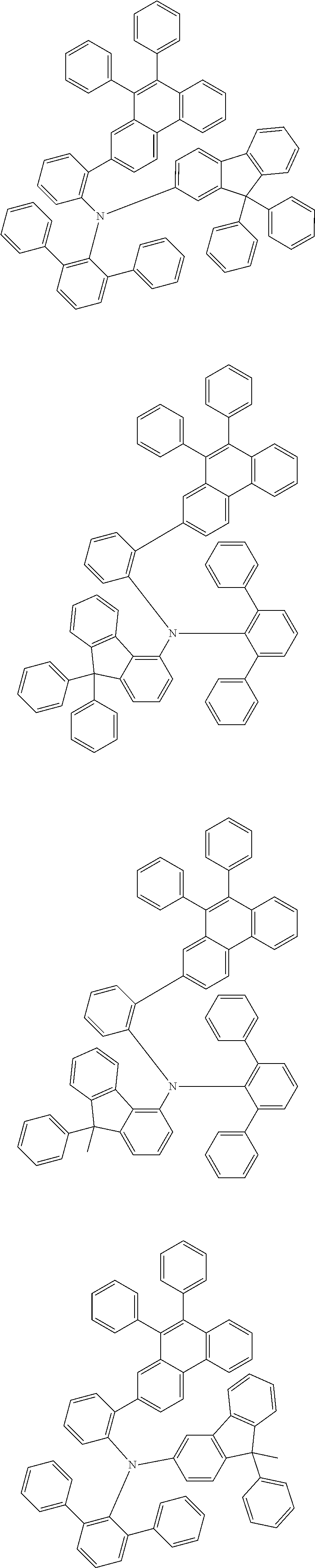 [SMILES: c1ccc(-c2cccc(-c3ccccc3)c2N(c2ccc3c(c2)C(c2ccccc2)(c2ccccc2)c2ccccc2-3)c2ccccc2-c2ccc3c(c2)c(-c2ccccc2)c(-c2ccccc2)c2ccccc23)cc1 ; c1ccc(-c2cccc(-c3ccccc3)c2N(c2ccccc2-c2ccc3c(c2)c(-c2ccccc2)c(-c2ccccc2)c2ccccc23)c2cccc3c2-c2ccccc2C3(c2ccccc2)c2ccccc2)cc1 ; CC1(c2ccccc2)c2ccccc2-c2c(N(c3ccccc3-c3ccc4c(c3)c(-c3ccccc3)c(-c3ccccc3)c3ccccc34)c3c(-c4ccccc4)cccc3-c3ccccc3)cccc21 ; CC1(c2ccccc2)c2ccccc2-c2cc(N(c3ccccc3-c3ccc4c(c3)c(-c3ccccc3)c(-c3ccccc3)c3ccccc34)c3c(-c4ccccc4)cccc3-c3ccccc3)ccc21]

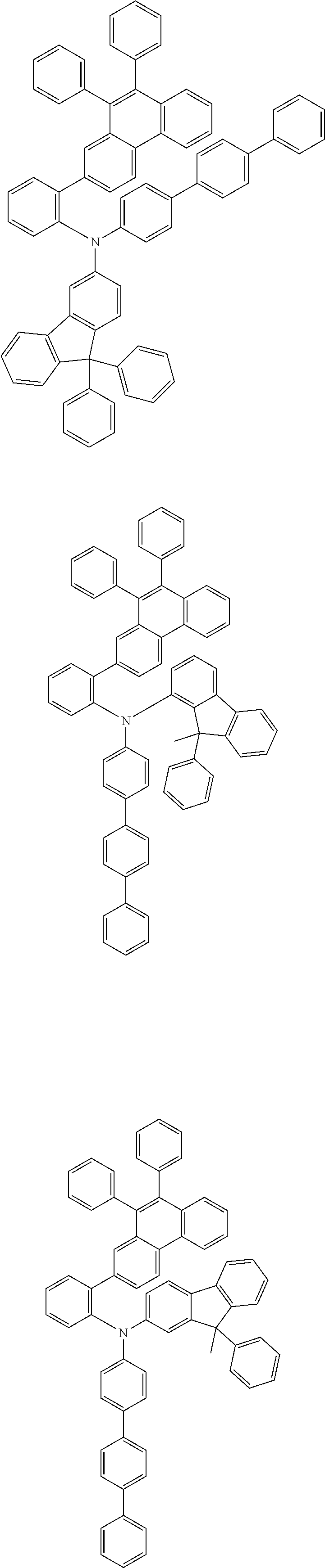 [SMILES: c1ccc(-c2ccc(-c3ccc(N(c4ccc5c(c4)-c4ccccc4C5(c4ccccc4)c4ccccc4)c4ccccc4-c4ccc5c(c4)c(-c4ccccc4)c(-c4ccccc4)c4ccccc45)cc3)cc2)cc1 ; CC1(c2ccccc2)c2ccccc2-c2cccc(N(c3ccc(-c4ccc(-c5ccccc5)cc4)cc3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4ccc(-c5ccccc5)cc4)cc3)c3ccccc3-c3ccc4c(c3)c(-c3ccccc3)c(-c3ccccc3)c3ccccc34)cc21]

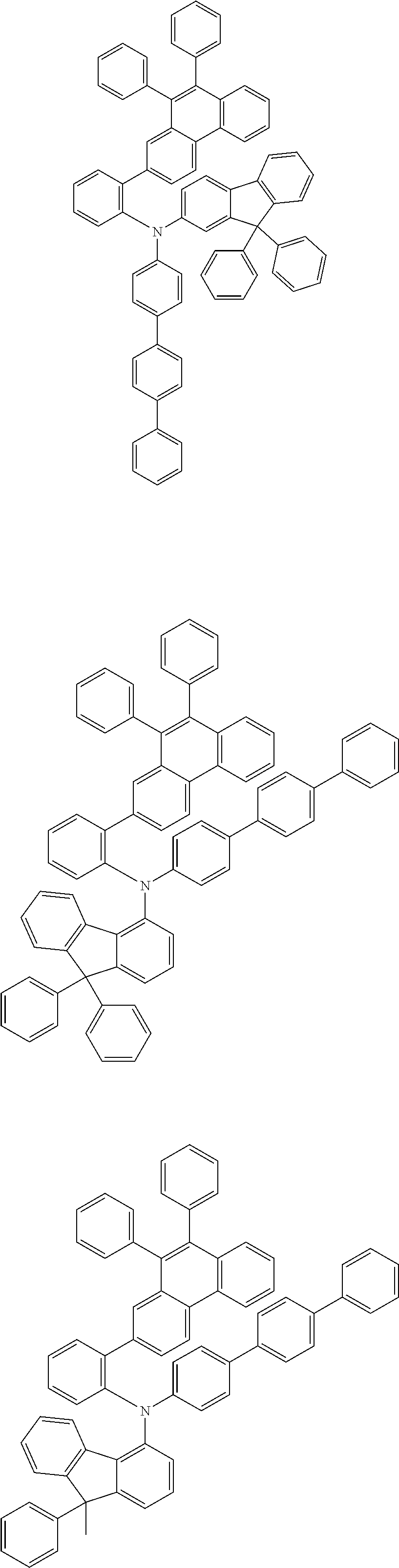 [SMILES: c1ccc(-c2ccc(-c3ccc(N(c4ccc5c(c4)C(c4ccccc4)(c4ccccc4)c4ccccc4-5)c4ccccc4-c4ccc5c(c4)c(-c4ccccc4)c(-c4ccccc4)c4ccccc45)cc3)cc2)cc1 ; c1ccc(-c2ccc(-c3ccc(N(c4ccccc4-c4ccc5c(c4)c(-c4ccccc4)c(-c4ccccc4)c4ccccc45)c4cccc5c4-c4ccccc4C5(c4ccccc4)c4ccccc4)cc3)cc2)cc1 ; CC1(c2ccccc2)c2ccccc2-c2c(N(c3ccc(-c4ccc(-c5ccccc5)cc4)cc3)c3ccccc3-c3ccc4c(c3)c(-c3ccccc3)c(-c3ccccc3)c3ccccc34)cccc21]

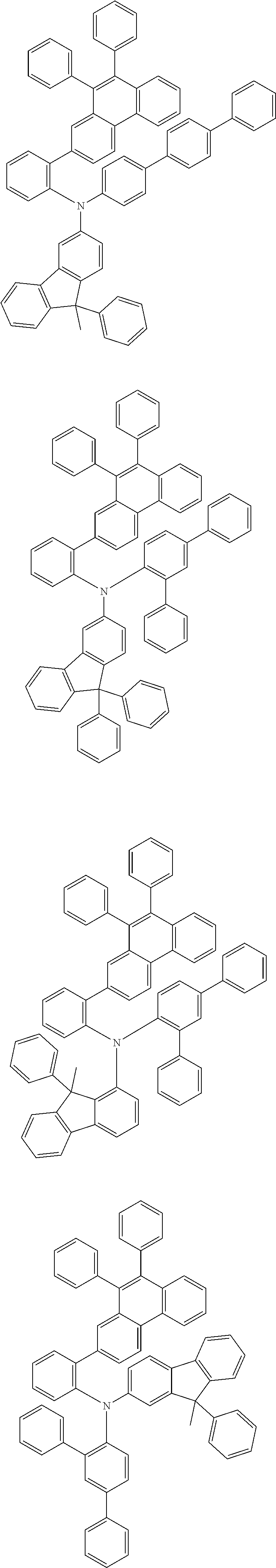 [SMILES: CC1(c2ccccc2)c2ccccc2-c2cc(N(c3ccc(-c4ccc(-c5ccccc5)cc4)cc3)c3ccccc3-c3ccc4c(c3)c(-c3ccccc3)c(-c3ccccc3)c3ccccc34)ccc21 ; c1ccc(-c2ccc(N(c3ccc4c(c3)-c3ccccc3C4(c3ccccc3)c3ccccc3)c3ccccc3-c3ccc4c(c3)c(-c3ccccc3)c(-c3ccccc3)c3ccccc34)c(-c3ccccc3)c2)cc1 ; CC1(c2ccccc2)c2ccccc2-c2cccc(N(c3ccccc3-c3ccc4c(c3)c(-c3ccccc3)c(-c3ccccc3)c3ccccc34)c3ccc(-c4ccccc4)cc3-c3ccccc3)c21 ; CC1(c2ccccc2)c2ccccc2-c2ccc(N(c3ccccc3-c3ccc4c(c3)c(-c3ccccc3)c(-c3ccccc3)c3ccccc34)c3ccc(-c4ccccc4)cc3-c3ccccc3)cc21]

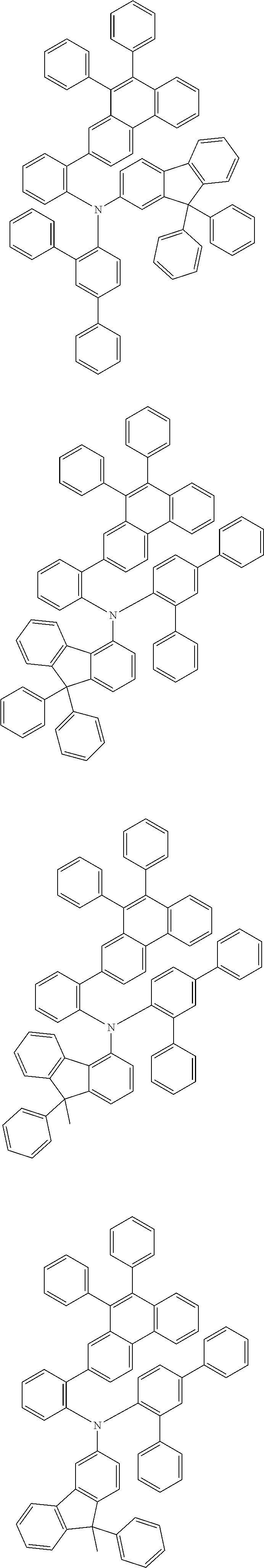 [SMILES: c1ccc(-c2ccc(N(c3ccc4c(c3)C(c3ccccc3)(c3ccccc3)c3ccccc3-4)c3ccccc3-c3ccc4c(c3)c(-c3ccccc3)c(-c3ccccc3)c3ccccc34)c(-c3ccccc3)c2)cc1 ; c1ccc(-c2ccc(N(c3ccccc3-c3ccc4c(c3)c(-c3ccccc3)c(-c3ccccc3)c3ccccc34)c3cccc4c3-c3ccccc3C4(c3ccccc3)c3ccccc3)c(-c3ccccc3)c2)cc1 ; CC1(c2ccccc2)c2ccccc2-c2c(N(c3ccccc3-c3ccc4c(c3)c(-c3ccccc3)c(-c3ccccc3)c3ccccc34)c3ccc(-c4ccccc4)cc3-c3ccccc3)cccc21 ; CC1(c2ccccc2)c2ccccc2-c2cc(N(c3ccccc3-c3ccc4c(c3)c(-c3ccccc3)c(-c3ccccc3)c3ccccc34)c3ccc(-c4ccccc4)cc3-c3ccccc3)ccc21]

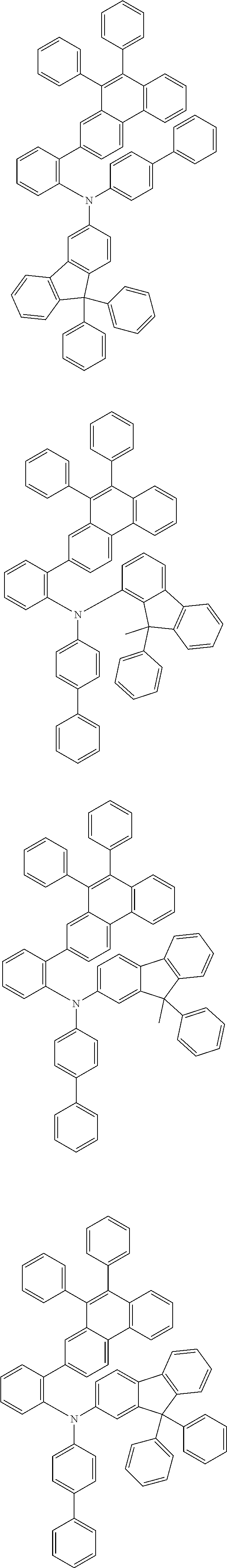 [SMILES: c1ccc(-c2ccc(N(c3ccc4c(c3)-c3ccccc3C4(c3ccccc3)c3ccccc3)c3ccccc3-c3ccc4c(c3)c(-c3ccccc3)c(-c3ccccc3)c3ccccc34)cc2)cc1 ; CC1(c2ccccc2)c2ccccc2-c2cccc(N(c3ccc(-c4ccccc4)cc3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4ccccc4)cc3)c3ccccc3-c3ccc4c(c3)c(-c3ccccc3)c(-c3ccccc3)c3ccccc34)cc21 ; c1ccc(-c2ccc(N(c3ccc4c(c3)C(c3ccccc3)(c3ccccc3)c3ccccc3-4)c3ccccc3-c3ccc4c(c3)c(-c3ccccc3)c(-c3ccccc3)c3ccccc34)cc2)cc1]

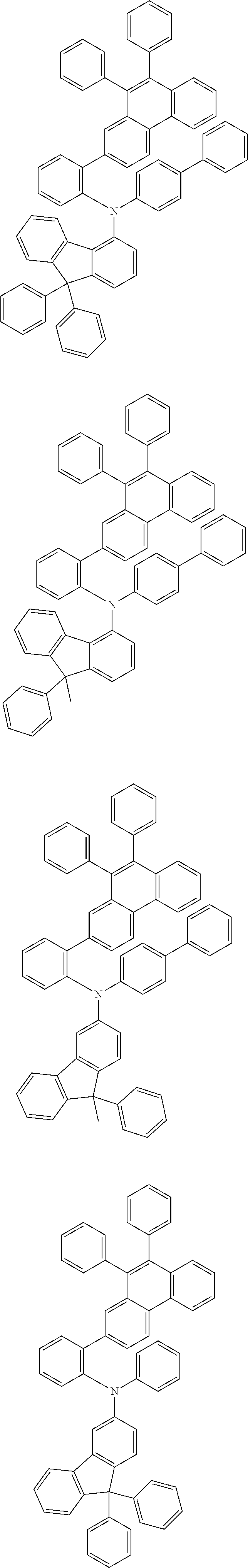 [SMILES: c1ccc(-c2ccc(N(c3ccccc3-c3ccc4c(c3)c(-c3ccccc3)c(-c3ccccc3)c3ccccc34)c3cccc4c3-c3ccccc3C4(c3ccccc3)c3ccccc3)cc2)cc1 ; CC1(c2ccccc2)c2ccccc2-c2c(N(c3ccc(-c4ccccc4)cc3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4ccccc4)cc3)c3ccccc3-c3ccc4c(c3)c(-c3ccccc3)c(-c3ccccc3)c3ccccc34)ccc21 ; c1ccc(-c2c(-c3ccccc3)c3cc(-c4ccccc4N(c4ccccc4)c4ccc5c(c4)-c4ccccc4C5(c4ccccc4)c4ccccc4)ccc3c3ccccc23)cc1]

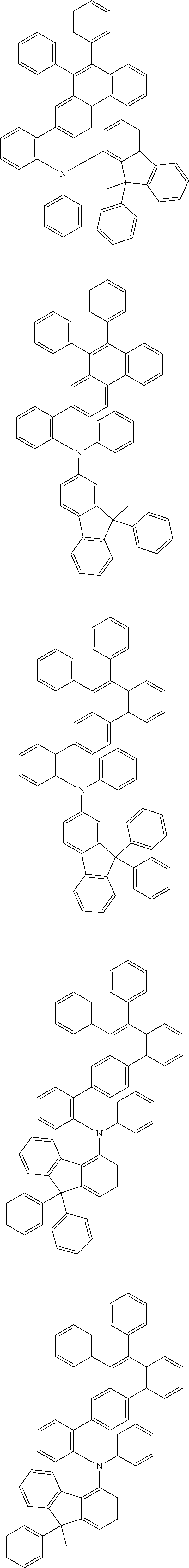

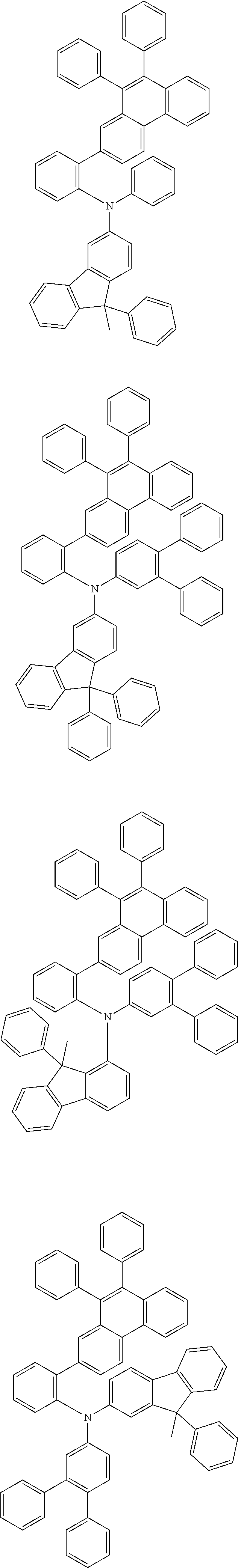 [SMILES: CC1(c2ccccc2)c2ccccc2-c2cc(N(c3ccccc3)c3ccccc3-c3ccc4c(c3)c(-c3ccccc3)c(-c3ccccc3)c3ccccc34)ccc21 ; c1ccc(-c2ccc(N(c3ccc4c(c3)-c3ccccc3C4(c3ccccc3)c3ccccc3)c3ccccc3-c3ccc4c(c3)c(-c3ccccc3)c(-c3ccccc3)c3ccccc34)cc2-c2ccccc2)cc1 ; CC1(c2ccccc2)c2ccccc2-c2cccc(N(c3ccc(-c4ccccc4)c(-c4ccccc4)c3)c3ccccc3-c3ccc4c(c3)c(-c3ccccc3)c(-c3ccccc3)c3ccccc34)c21 ; CC1(c2ccccc2)c2ccccc2-c2ccc(N(c3ccc(-c4ccccc4)c(-c4ccccc4)c3)c3ccccc3-c3ccc4c(c3)c(-c3ccccc3)c(-c3ccccc3)c3ccccc34)cc21]

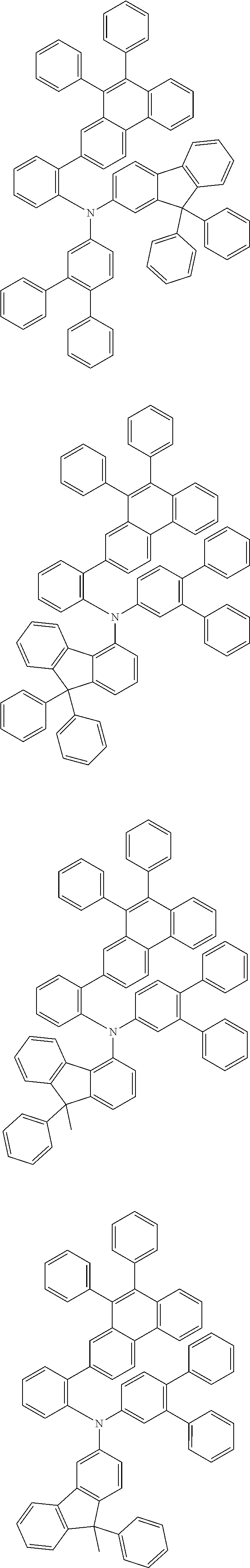 [SMILES: c1ccc(-c2ccc(N(c3ccc4c(c3)C(c3ccccc3)(c3ccccc3)c3ccccc3-4)c3ccccc3-c3ccc4c(c3)c(-c3ccccc3)c(-c3ccccc3)c3ccccc34)cc2-c2ccccc2)cc1 ; c1ccc(-c2ccc(N(c3ccccc3-c3ccc4c(c3)c(-c3ccccc3)c(-c3ccccc3)c3ccccc34)c3cccc4c3-c3ccccc3C4(c3ccccc3)c3ccccc3)cc2-c2ccccc2)cc1 ; CC1(c2ccccc2)c2ccccc2-c2c(N(c3ccc(-c4ccccc4)c(-c4ccccc4)c3)c3ccccc3-c3ccc4c(c3)c(-c3ccccc3)c(-c3ccccc3)c3ccccc34)cccc21 ; CC1(c2ccccc2)c2ccccc2-c2cc(N(c3ccc(-c4ccccc4)c(-c4ccccc4)c3)c3ccccc3-c3ccc4c(c3)c(-c3ccccc3)c(-c3ccccc3)c3ccccc34)ccc21]

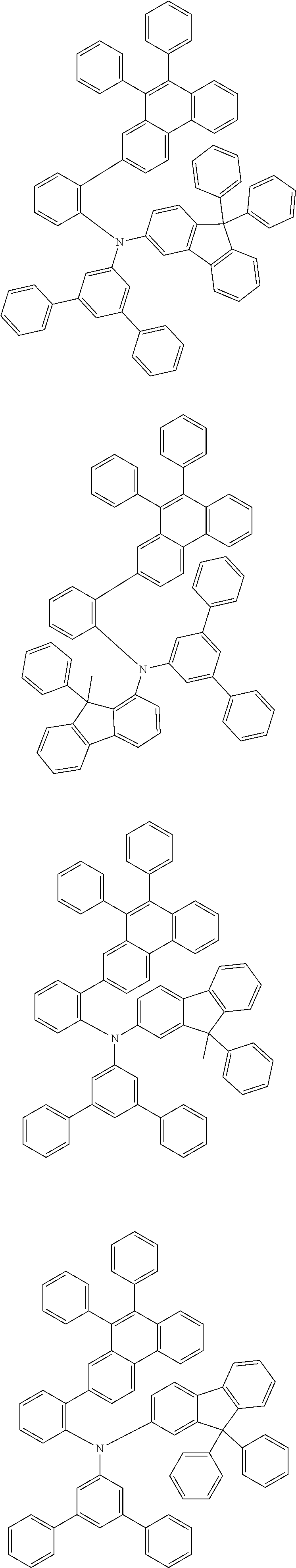

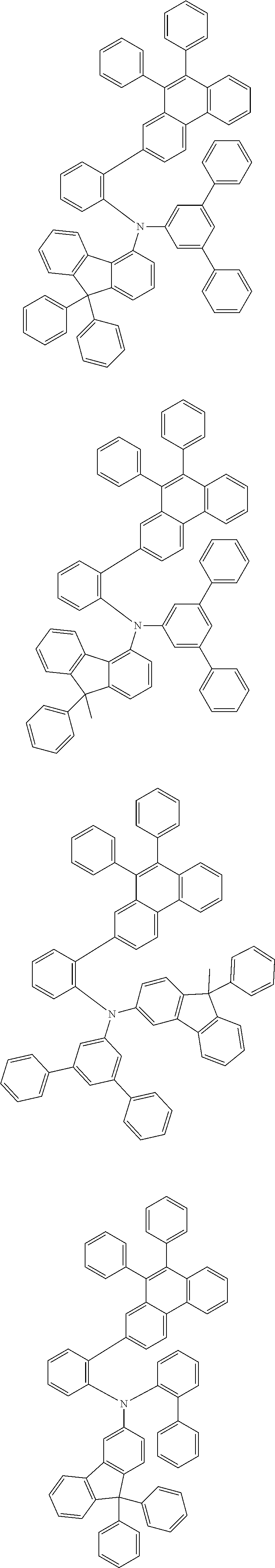 [SMILES: c1ccc(-c2cc(-c3ccccc3)cc(N(c3ccccc3-c3ccc4c(c3)c(-c3ccccc3)c(-c3ccccc3)c3ccccc34)c3cccc4c3-c3ccccc3C4(c3ccccc3)c3ccccc3)c2)cc1 ; CC1(c2ccccc2)c2ccccc2-c2c(N(c3cc(-c4ccccc4)cc(-c4ccccc4)c3)c3ccccc3-c3ccc4c(c3)c(-c3ccccc3)c(-c3ccccc3)c3ccccc34)cccc21 ; CC1(c2ccccc2)c2ccccc2-c2cc(N(c3cc(-c4ccccc4)cc(-c4ccccc4)c3)c3ccccc3-c3ccc4c(c3)c(-c3ccccc3)c(-c3ccccc3)c3ccccc34)ccc21 ; c1ccc(-c2ccccc2N(c2ccc3c(c2)-c2ccccc2C3(c2ccccc2)c2ccccc2)c2ccccc2-c2ccc3c(c2)c(-c2ccccc2)c(-c2ccccc2)c2ccccc23)cc1]

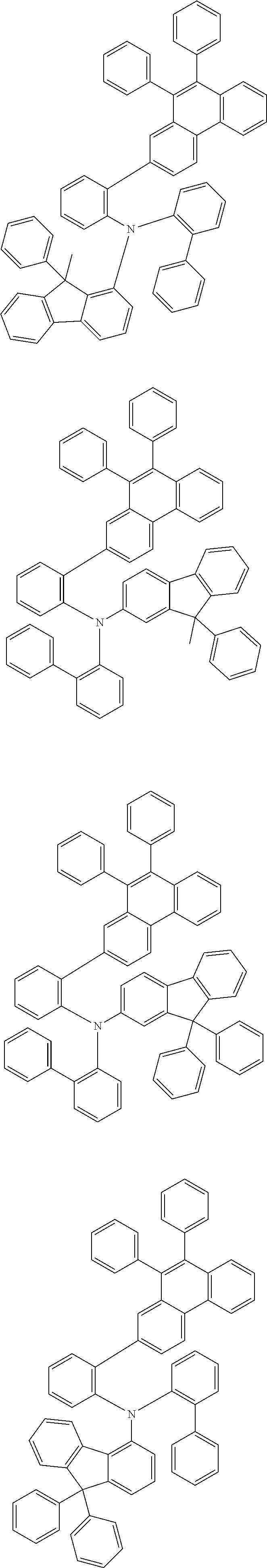 [SMILES: CC1(c2ccccc2)c2ccccc2-c2cccc(N(c3ccccc3-c3ccccc3)c3ccccc3-c3ccc4c(c3)c(-c3ccccc3)c(-c3ccccc3)c3ccccc34)c21 ; CC1(c2ccccc2)c2ccccc2-c2ccc(N(c3ccccc3-c3ccccc3)c3ccccc3-c3ccc4c(c3)c(-c3ccccc3)c(-c3ccccc3)c3ccccc34)cc21 ; c1ccc(-c2ccccc2N(c2ccc3c(c2)C(c2ccccc2)(c2ccccc2)c2ccccc2-3)c2ccccc2-c2ccc3c(c2)c(-c2ccccc2)c(-c2ccccc2)c2ccccc23)cc1 ; c1ccc(-c2ccccc2N(c2ccccc2-c2ccc3c(c2)c(-c2ccccc2)c(-c2ccccc2)c2ccccc23)c2cccc3c2-c2ccccc2C3(c2ccccc2)c2ccccc2)cc1]

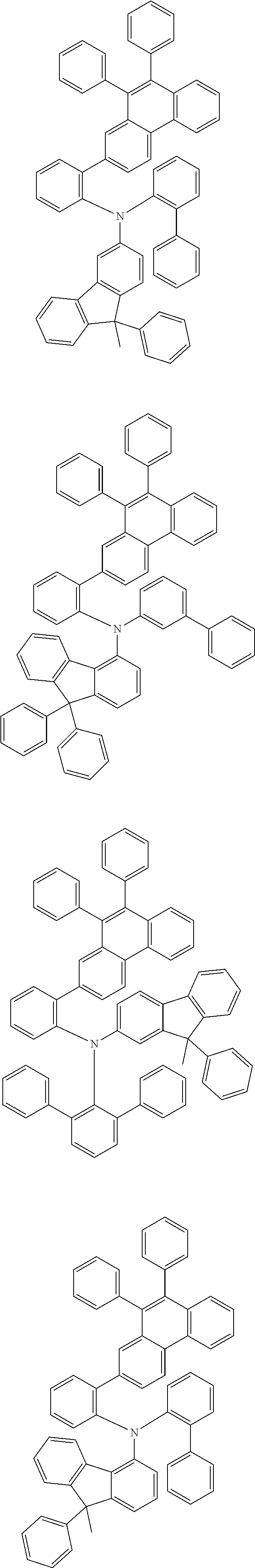 [SMILES: CC1(c2ccccc2)c2ccccc2-c2cc(N(c3ccccc3-c3ccccc3)c3ccccc3-c3ccc4c(c3)c(-c3ccccc3)c(-c3ccccc3)c3ccccc34)ccc21 ; c1ccc(-c2cccc(N(c3ccccc3-c3ccc4c(c3)c(-c3ccccc3)c(-c3ccccc3)c3ccccc34)c3cccc4c3-c3ccccc3C4(c3ccccc3)c3ccccc3)c2)cc1 ; CC1(c2ccccc2)c2ccccc2-c2ccc(N(c3ccccc3-c3ccc4c(c3)c(-c3ccccc3)c(-c3ccccc3)c3ccccc34)c3c(-c4ccccc4)cccc3-c3ccccc3)cc21 ; CC1(c2ccccc2)c2ccccc2-c2c(N(c3ccccc3-c3ccccc3)c3ccccc3-c3ccc4c(c3)c(-c3ccccc3)c(-c3ccccc3)c3ccccc34)cccc21]

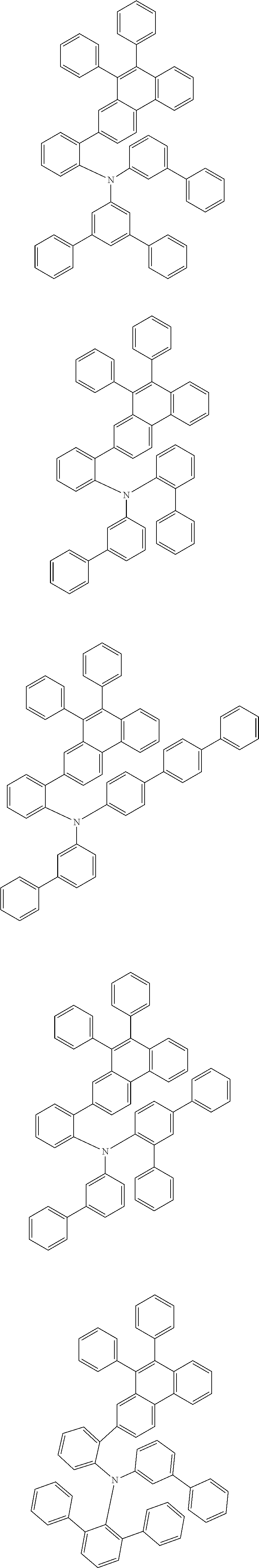

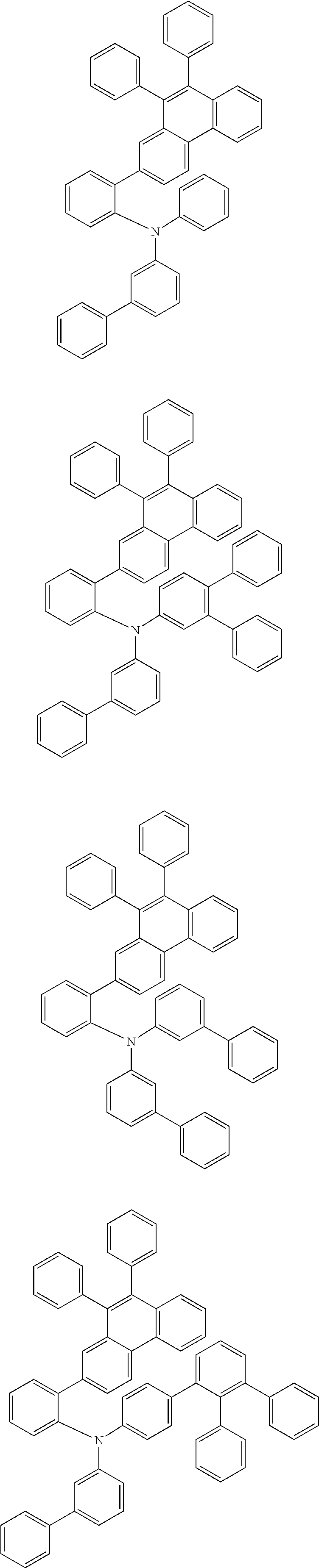 [SMILES: c1ccc(-c2cccc(N(c3ccccc3)c3ccccc3-c3ccc4c(c3)c(-c3ccccc3)c(-c3ccccc3)c3ccccc34)c2)cc1 ; c1ccc(-c2cccc(N(c3ccc(-c4ccccc4)c(-c4ccccc4)c3)c3ccccc3-c3ccc4c(c3)c(-c3ccccc3)c(-c3ccccc3)c3ccccc34)c2)cc1 ; c1ccc(-c2cccc(N(c3cccc(-c4ccccc4)c3)c3ccccc3-c3ccc4c(c3)c(-c3ccccc3)c(-c3ccccc3)c3ccccc34)c2)cc1 ; c1ccc(-c2cccc(N(c3ccc(-c4cccc(-c5ccccc5)c4-c4ccccc4)cc3)c3ccccc3-c3ccc4c(c3)c(-c3ccccc3)c(-c3ccccc3)c3ccccc34)c2)cc1]

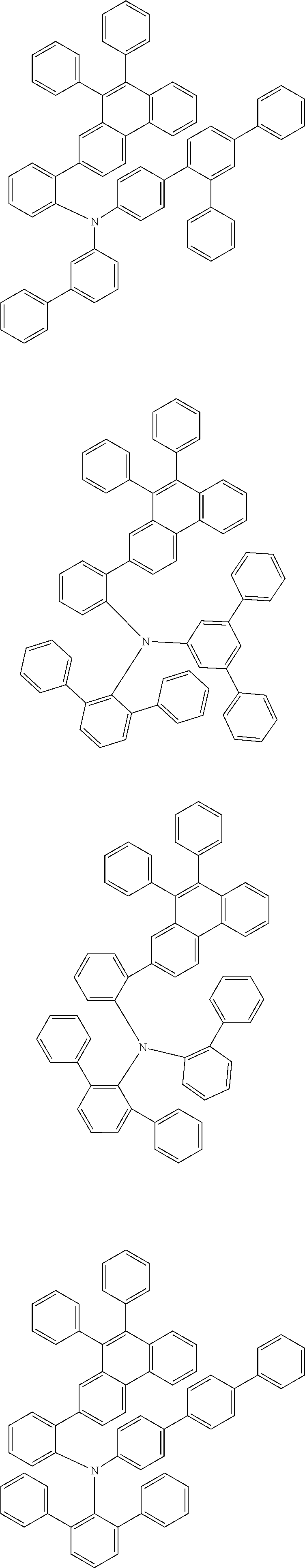 [SMILES: c1ccc(-c2cccc(N(c3ccc(-c4ccc(-c5ccccc5)cc4-c4ccccc4)cc3)c3ccccc3-c3ccc4c(c3)c(-c3ccccc3)c(-c3ccccc3)c3ccccc34)c2)cc1 ; c1ccc(-c2cc(-c3ccccc3)cc(N(c3ccccc3-c3ccc4c(c3)c(-c3ccccc3)c(-c3ccccc3)c3ccccc34)c3c(-c4ccccc4)cccc3-c3ccccc3)c2)cc1 ; c1ccc(-c2ccccc2N(c2ccccc2-c2ccc3c(c2)c(-c2ccccc2)c(-c2ccccc2)c2ccccc23)c2c(-c3ccccc3)cccc2-c2ccccc2)cc1 ; c1ccc(-c2ccc(-c3ccc(N(c4ccccc4-c4ccc5c(c4)c(-c4ccccc4)c(-c4ccccc4)c4ccccc45)c4c(-c5ccccc5)cccc4-c4ccccc4)cc3)cc2)cc1]

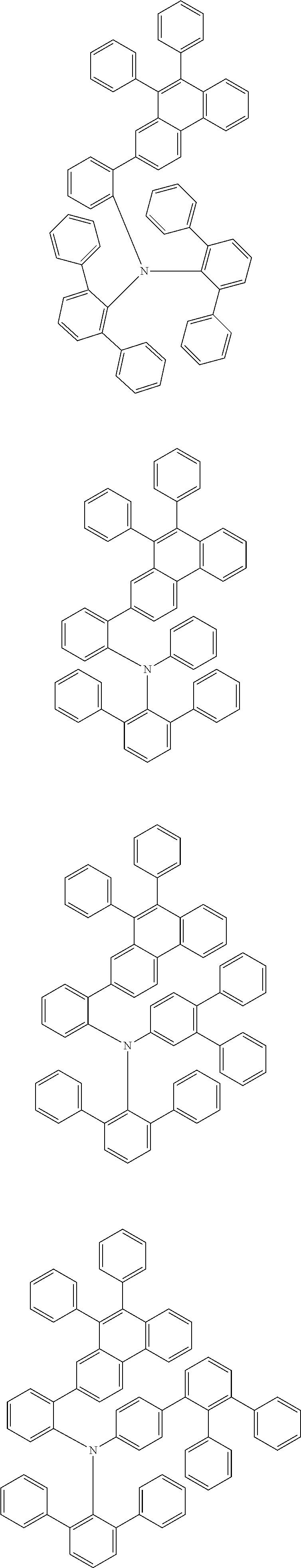 [SMILES: c1ccc(-c2cccc(-c3ccccc3)c2N(c2ccccc2-c2ccc3c(c2)c(-c2ccccc2)c(-c2ccccc2)c2ccccc23)c2c(-c3ccccc3)cccc2-c2ccccc2)cc1 ; c1ccc(-c2cccc(-c3ccccc3)c2N(c2ccccc2)c2ccccc2-c2ccc3c(c2)c(-c2ccccc2)c(-c2ccccc2)c2ccccc23)cc1 ; c1ccc(-c2ccc(N(c3ccccc3-c3ccc4c(c3)c(-c3ccccc3)c(-c3ccccc3)c3ccccc34)c3c(-c4ccccc4)cccc3-c3ccccc3)cc2-c2ccccc2)cc1 ; c1ccc(-c2cccc(-c3ccc(N(c4ccccc4-c4ccc5c(c4)c(-c4ccccc4)c(-c4ccccc4)c4ccccc45)c4c(-c5ccccc5)cccc4-c4ccccc4)cc3)c2-c2ccccc2)cc1]

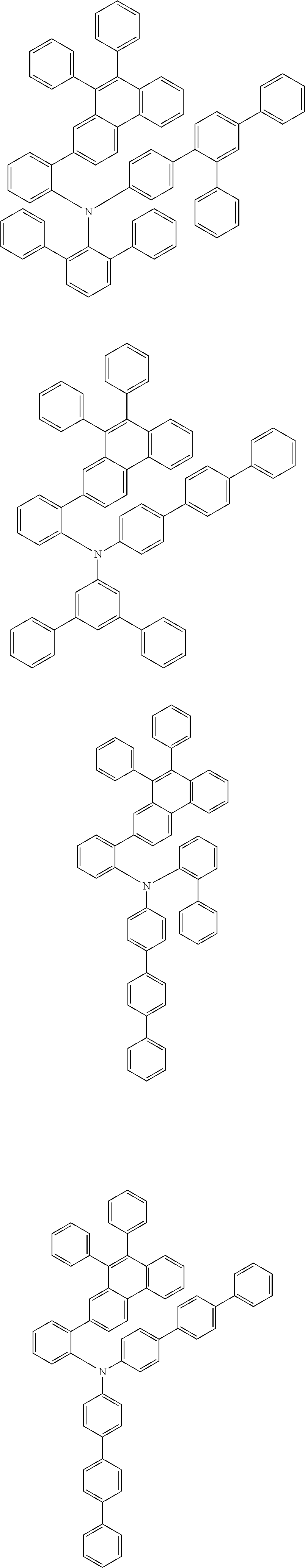 [SMILES: c1ccc(-c2ccc(-c3ccc(N(c4ccccc4-c4ccc5c(c4)c(-c4ccccc4)c(-c4ccccc4)c4ccccc45)c4c(-c5ccccc5)cccc4-c4ccccc4)cc3)c(-c3ccccc3)c2)cc1 ; c1ccc(-c2ccc(-c3ccc(N(c4cc(-c5ccccc5)cc(-c5ccccc5)c4)c4ccccc4-c4ccc5c(c4)c(-c4ccccc4)c(-c4ccccc4)c4ccccc45)cc3)cc2)cc1 ; c1ccc(-c2ccc(-c3ccc(N(c4ccccc4-c4ccccc4)c4ccccc4-c4ccc5c(c4)c(-c4ccccc4)c(-c4ccccc4)c4ccccc45)cc3)cc2)cc1 ; c1ccc(-c2ccc(-c3ccc(N(c4ccc(-c5ccc(-c6ccccc6)cc5)cc4)c4ccccc4-c4ccc5c(c4)c(-c4ccccc4)c(-c4ccccc4)c4ccccc45)cc3)cc2)cc1]

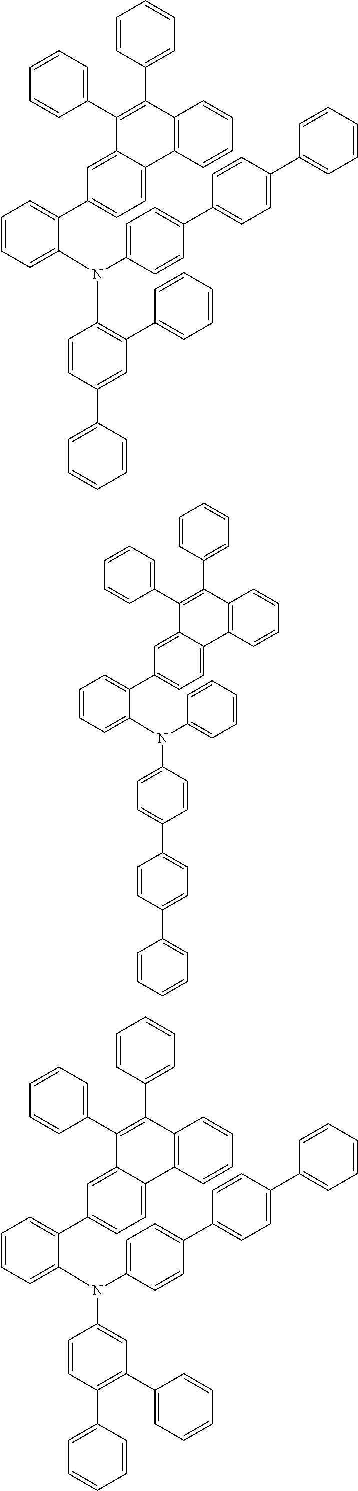

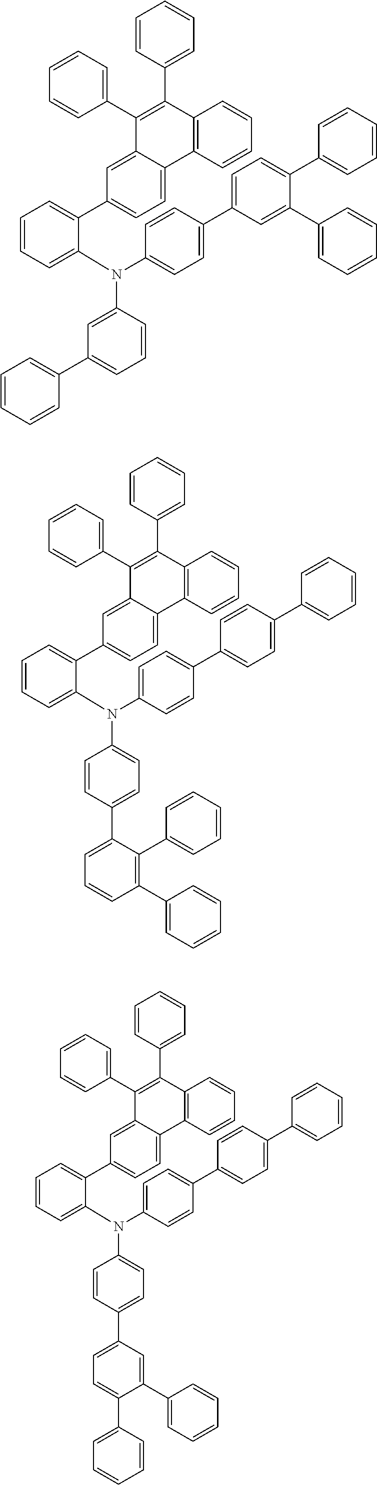 [SMILES: c1ccc(-c2cccc(N(c3ccc(-c4ccc(-c5ccccc5)c(-c5ccccc5)c4)cc3)c3ccccc3-c3ccc4c(c3)c(-c3ccccc3)c(-c3ccccc3)c3ccccc34)c2)cc1 ; c1ccc(-c2ccc(-c3ccc(N(c4ccc(-c5cccc(-c6ccccc6)c5-c5ccccc5)cc4)c4ccccc4-c4ccc5c(c4)c(-c4ccccc4)c(-c4ccccc4)c4ccccc45)cc3)cc2)cc1 ; c1ccc(-c2ccc(-c3ccc(N(c4ccc(-c5ccc(-c6ccccc6)c(-c6ccccc6)c5)cc4)c4ccccc4-c4ccc5c(c4)c(-c4ccccc4)c(-c4ccccc4)c4ccccc45)cc3)cc2)cc1]

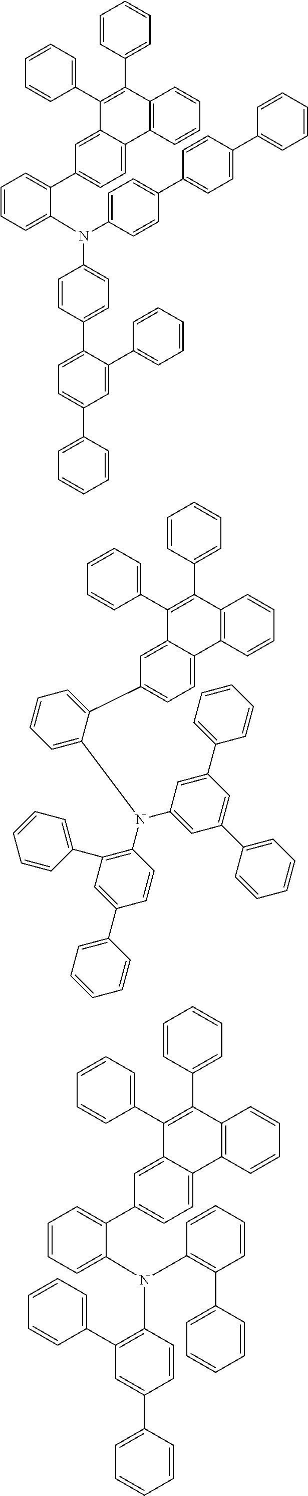 [SMILES: c1ccc(-c2ccc(-c3ccc(N(c4ccc(-c5ccc(-c6ccccc6)cc5-c5ccccc5)cc4)c4ccccc4-c4ccc5c(c4)c(-c4ccccc4)c(-c4ccccc4)c4ccccc45)cc3)cc2)cc1 ; c1ccc(-c2cc(-c3ccccc3)cc(N(c3ccccc3-c3ccc4c(c3)c(-c3ccccc3)c(-c3ccccc3)c3ccccc34)c3ccc(-c4ccccc4)cc3-c3ccccc3)c2)cc1 ; c1ccc(-c2ccc(N(c3ccccc3-c3ccccc3)c3ccccc3-c3ccc4c(c3)c(-c3ccccc3)c(-c3ccccc3)c3ccccc34)c(-c3ccccc3)c2)cc1]

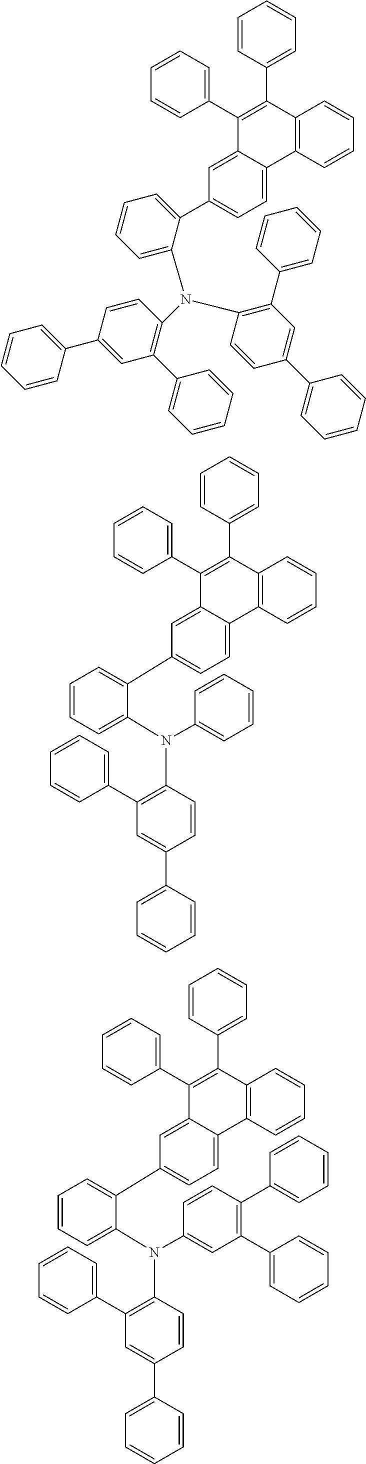

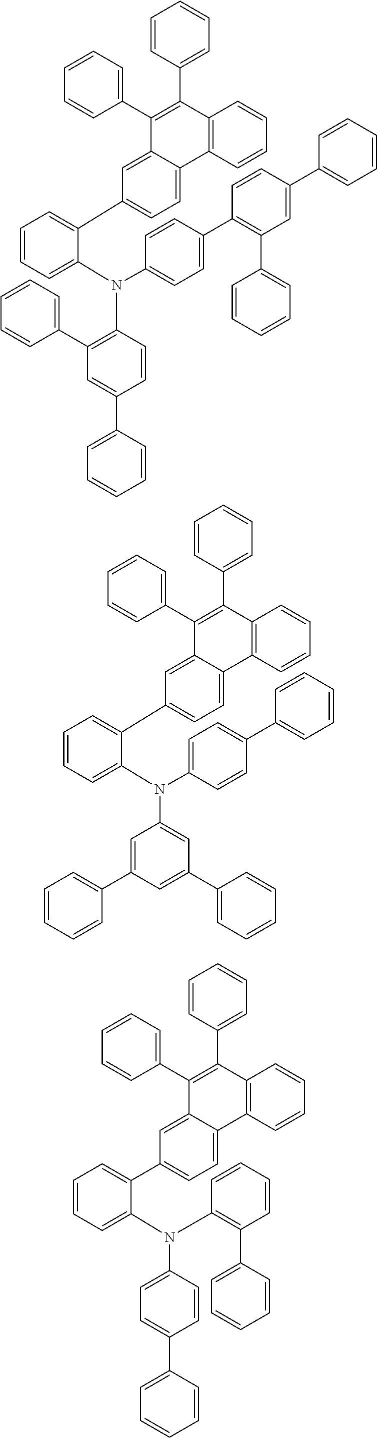

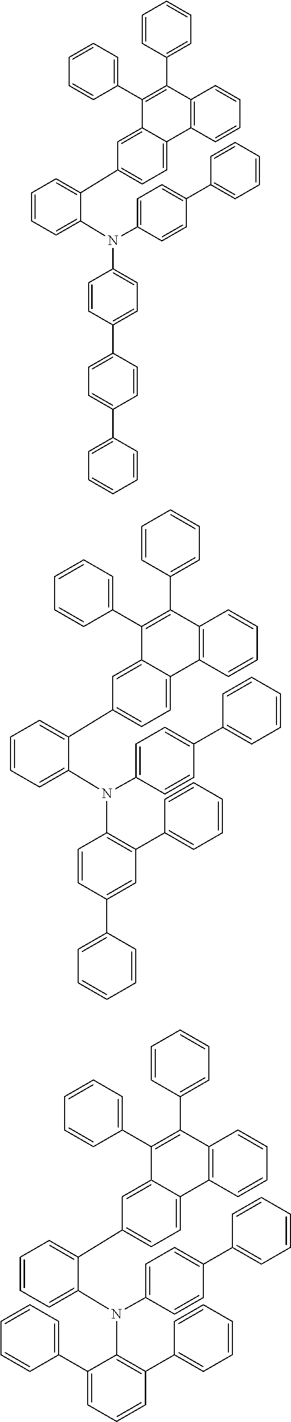

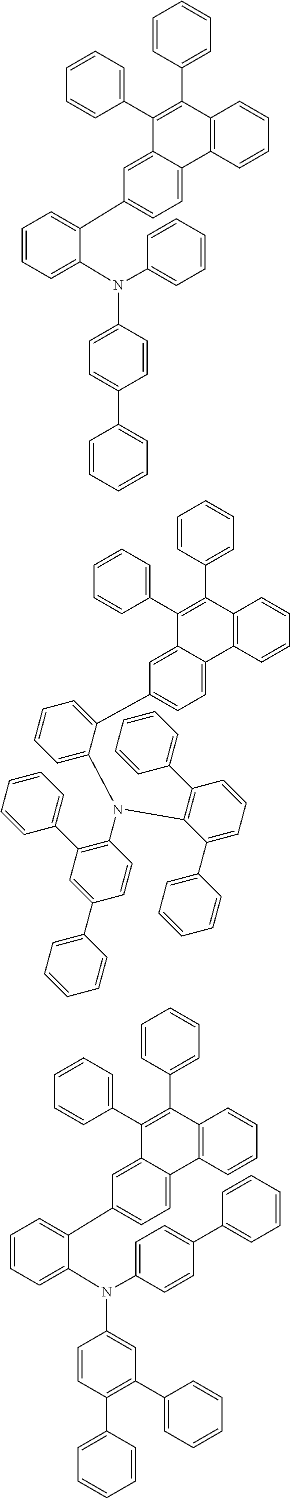 [SMILES: c1ccc(-c2ccc(N(c3ccccc3)c3ccccc3-c3ccc4c(c3)c(-c3ccccc3)c(-c3ccccc3)c3ccccc34)cc2)cc1 ; c1ccc(-c2ccc(N(c3ccccc3-c3ccc4c(c3)c(-c3ccccc3)c(-c3ccccc3)c3ccccc34)c3c(-c4ccccc4)cccc3-c3ccccc3)c(-c3ccccc3)c2)cc1 ; c1ccc(-c2ccc(N(c3ccc(-c4ccccc4)c(-c4ccccc4)c3)c3ccccc3-c3ccc4c(c3)c(-c3ccccc3)c(-c3ccccc3)c3ccccc34)cc2)cc1]

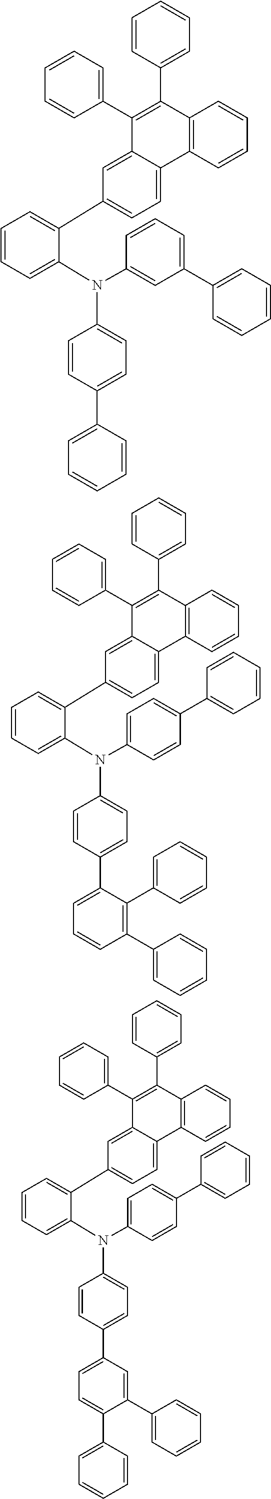

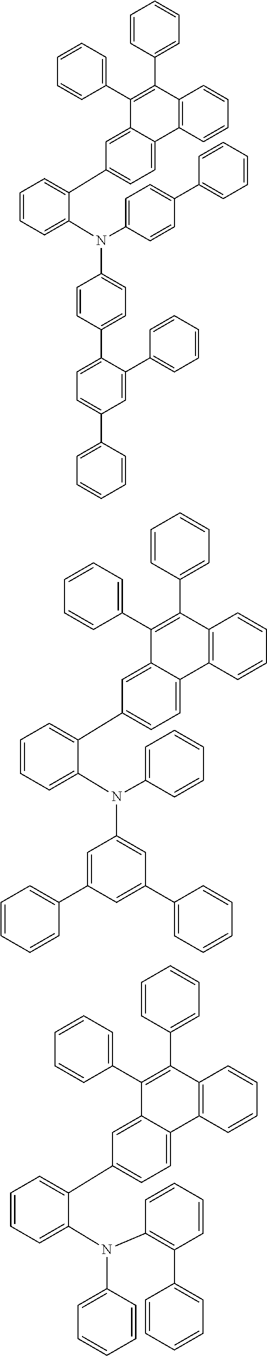

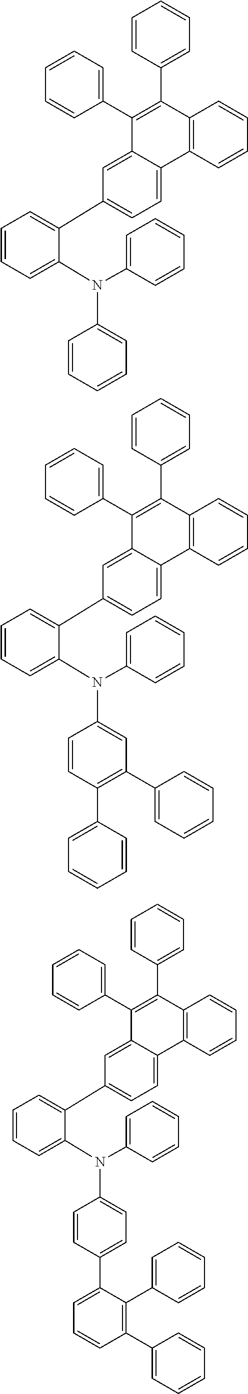

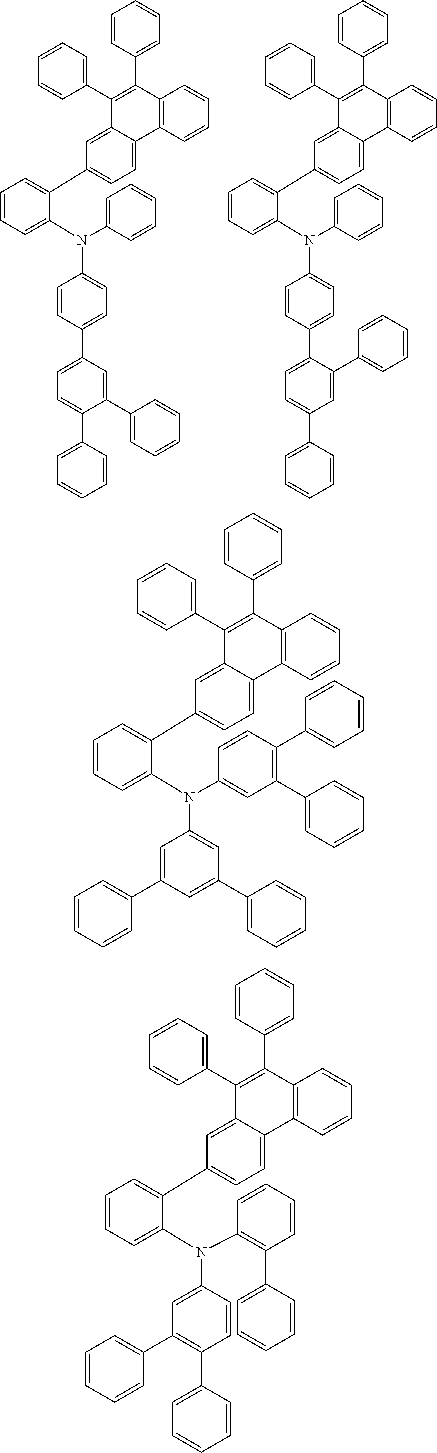

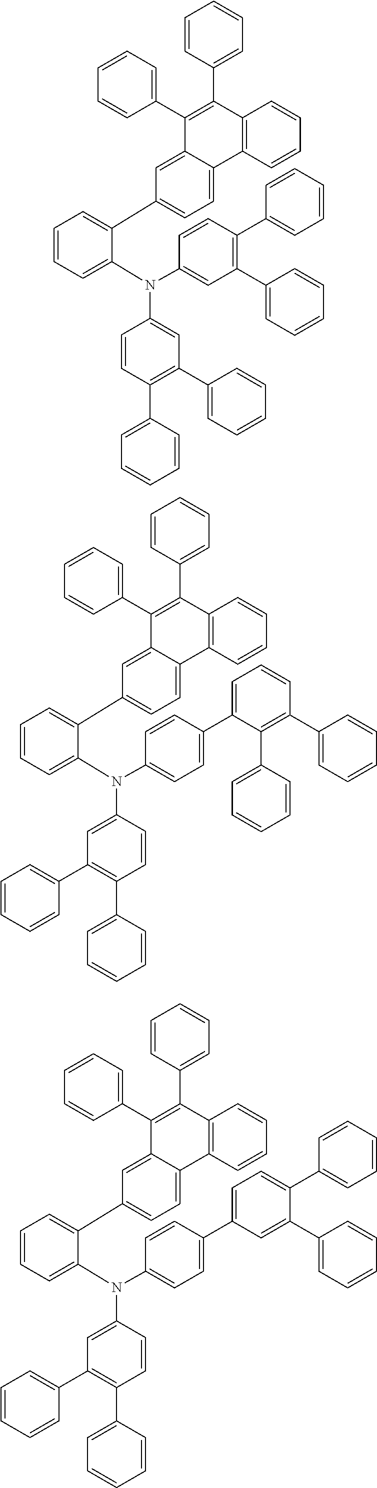

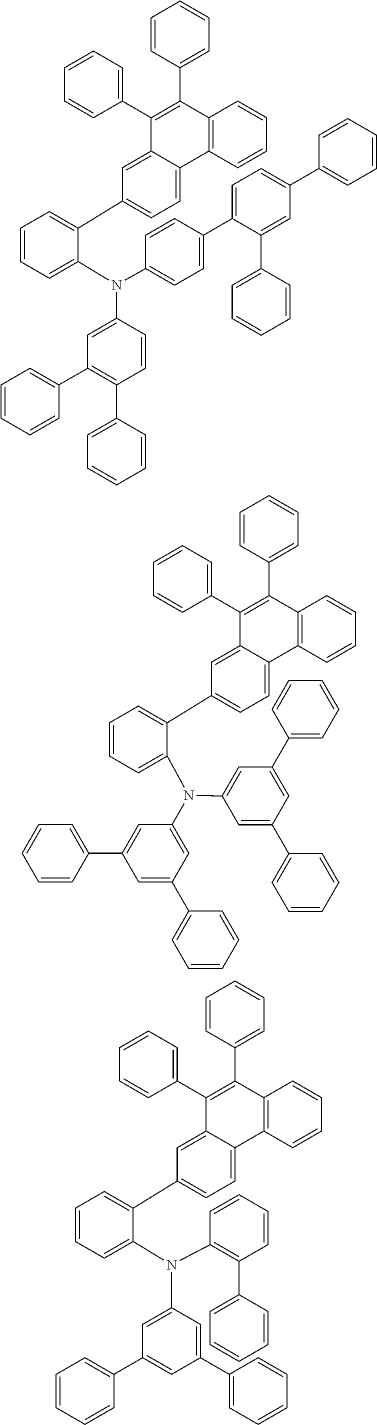

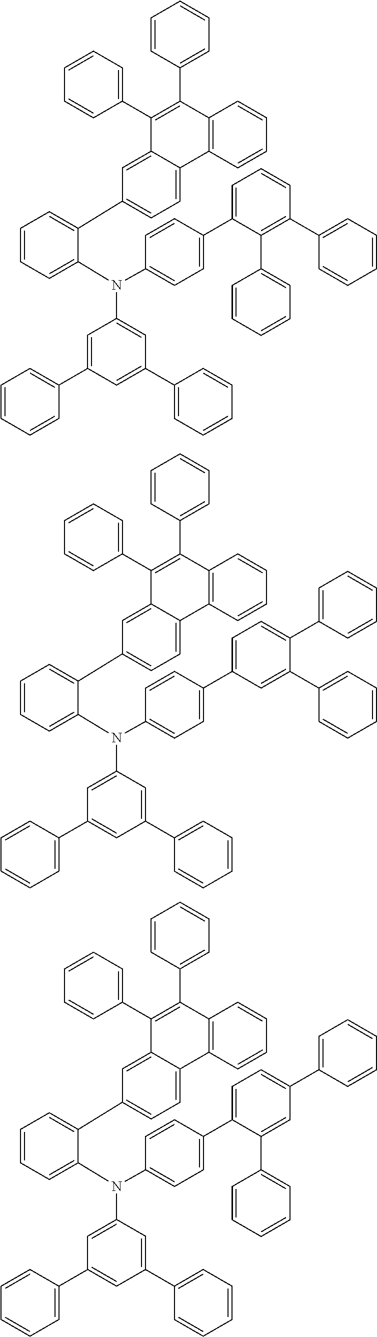

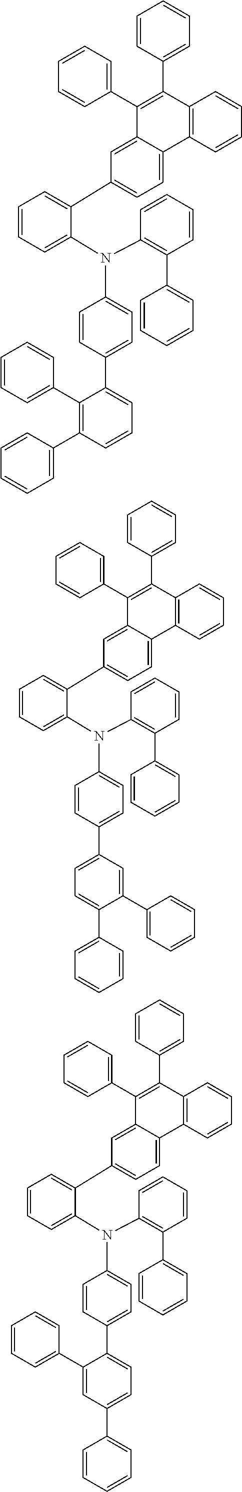

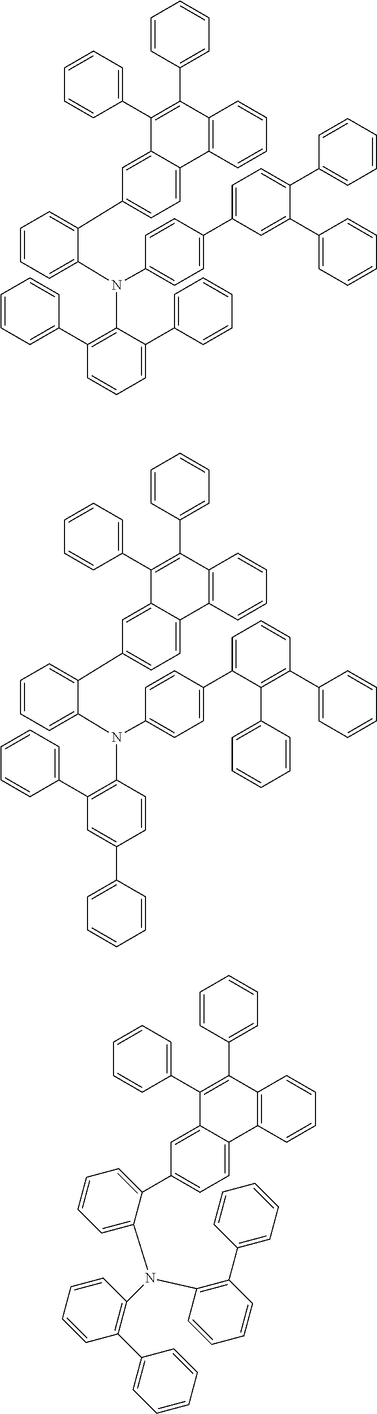 [SMILES: c1ccc(-c2ccc(-c3ccc(N(c4ccccc4-c4ccc5c(c4)c(-c4ccccc4)c(-c4ccccc4)c4ccccc45)c4c(-c5ccccc5)cccc4-c4ccccc4)cc3)cc2-c2ccccc2)cc1 ; c1ccc(-c2ccc(N(c3ccc(-c4cccc(-c5ccccc5)c4-c4ccccc4)cc3)c3ccccc3-c3ccc4c(c3)c(-c3ccccc3)c(-c3ccccc3)c3ccccc34)c(-c3ccccc3)c2)cc1 ; c1ccc(-c2ccccc2N(c2ccccc2-c2ccccc2)c2ccccc2-c2ccc3c(c2)c(-c2ccccc2)c(-c2ccccc2)c2ccccc23)cc1]

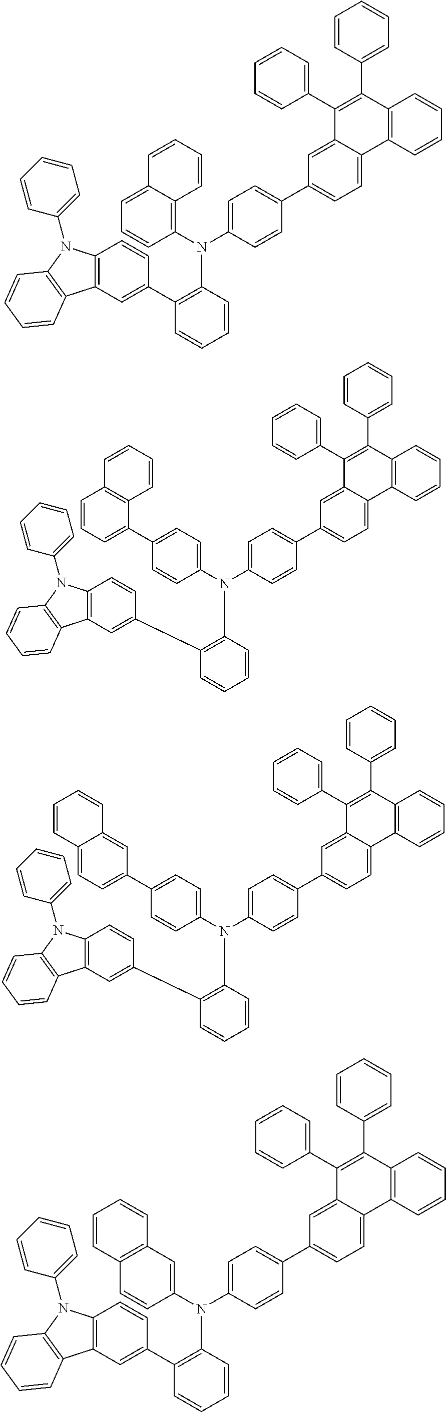 [SMILES: c1ccc(-c2c(-c3ccccc3)c3cc(-c4ccc(N(c5ccccc5-c5ccc6c(c5)c5ccccc5n6-c5ccccc5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccccc5-c5ccc6c(c5)c5ccccc5n6-c5ccccc5)cc4)ccc3c3ccccc23)cc1 ; c1ccc(-c2c(-c3ccccc3)c3cc(-c4ccc(N(c5ccc(-c6ccc7ccccc7c6)cc5)c5ccccc5-c5ccc6c(c5)c5ccccc5n6-c5ccccc5)cc4)ccc3c3ccccc23)cc1 ; c1ccc(-c2c(-c3ccccc3)c3cc(-c4ccc(N(c5ccc6ccccc6c5)c5ccccc5-c5ccc6c(c5)c5ccccc5n6-c5ccccc5)cc4)ccc3c3ccccc23)cc1]

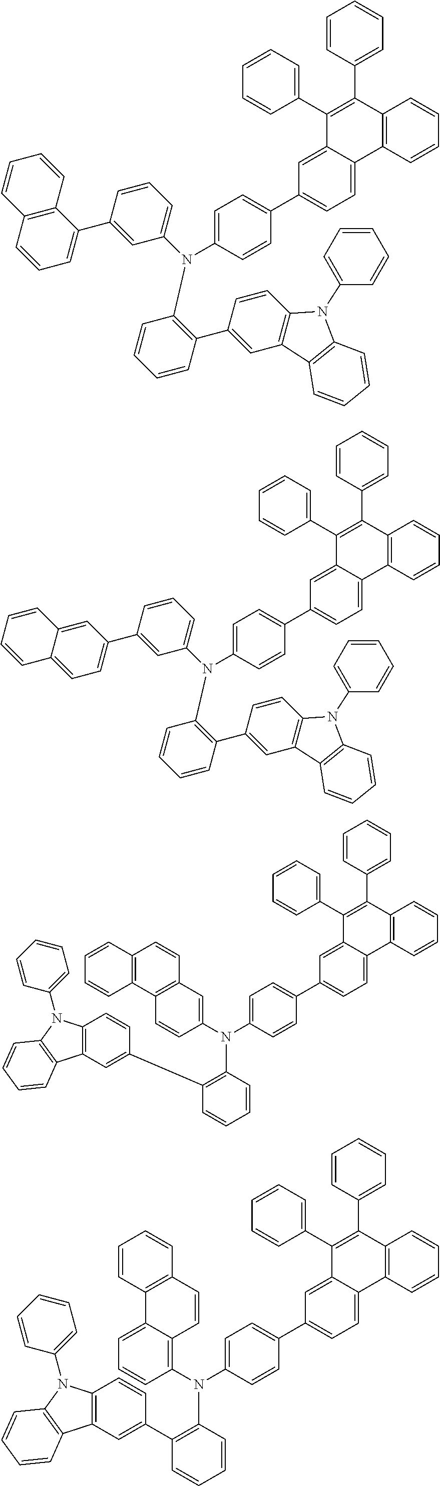

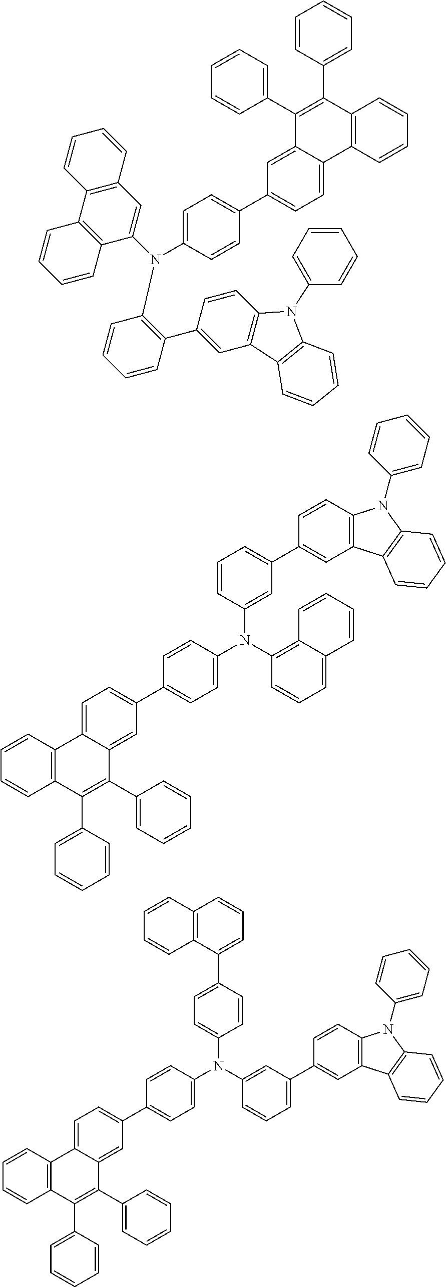 [SMILES: c1ccc(-c2c(-c3ccccc3)c3cc(-c4ccc(N(c5ccccc5-c5ccc6c(c5)c5ccccc5n6-c5ccccc5)c5cc6ccccc6c6ccccc56)cc4)ccc3c3ccccc23)cc1 ; c1ccc(-c2c(-c3ccccc3)c3cc(-c4ccc(N(c5cccc(-c6ccc7c(c6)c6ccccc6n7-c6ccccc6)c5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5cccc(-c6ccc7c(c6)c6ccccc6n7-c6ccccc6)c5)cc4)ccc3c3ccccc23)cc1]

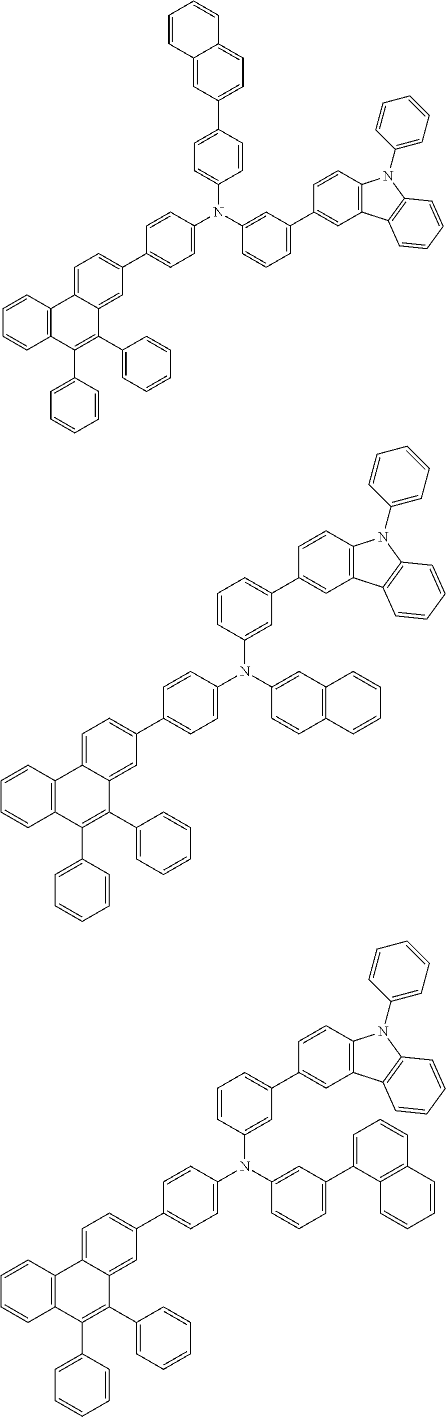 [SMILES: c1ccc(-c2c(-c3ccccc3)c3cc(-c4ccc(N(c5ccc(-c6ccc7ccccc7c6)cc5)c5cccc(-c6ccc7c(c6)c6ccccc6n7-c6ccccc6)c5)cc4)ccc3c3ccccc23)cc1 ; c1ccc(-c2c(-c3ccccc3)c3cc(-c4ccc(N(c5cccc(-c6ccc7c(c6)c6ccccc6n7-c6ccccc6)c5)c5ccc6ccccc6c5)cc4)ccc3c3ccccc23)cc1 ; c1ccc(-c2c(-c3ccccc3)c3cc(-c4ccc(N(c5cccc(-c6ccc7c(c6)c6ccccc6n7-c6ccccc6)c5)c5cccc(-c6cccc7ccccc67)c5)cc4)ccc3c3ccccc23)cc1]

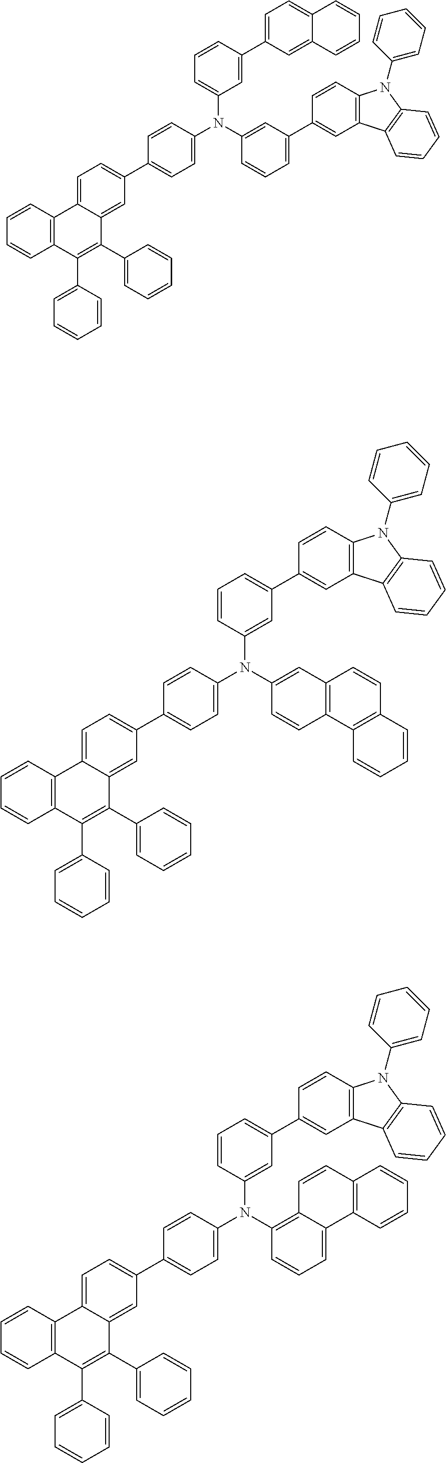 [SMILES: c1ccc(-c2c(-c3ccccc3)c3cc(-c4ccc(N(c5cccc(-c6ccc7ccccc7c6)c5)c5cccc(-c6ccc7c(c6)c6ccccc6n7-c6ccccc6)c5)cc4)ccc3c3ccccc23)cc1 ; c1ccc(-c2c(-c3ccccc3)c3cc(-c4ccc(N(c5cccc(-c6ccc7c(c6)c6ccccc6n7-c6ccccc6)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6ccc7c(c6)c6ccccc6n7-c6ccccc6)c5)c5cccc6c5ccc5ccccc56)cc4)ccc3c3ccccc23)cc1]

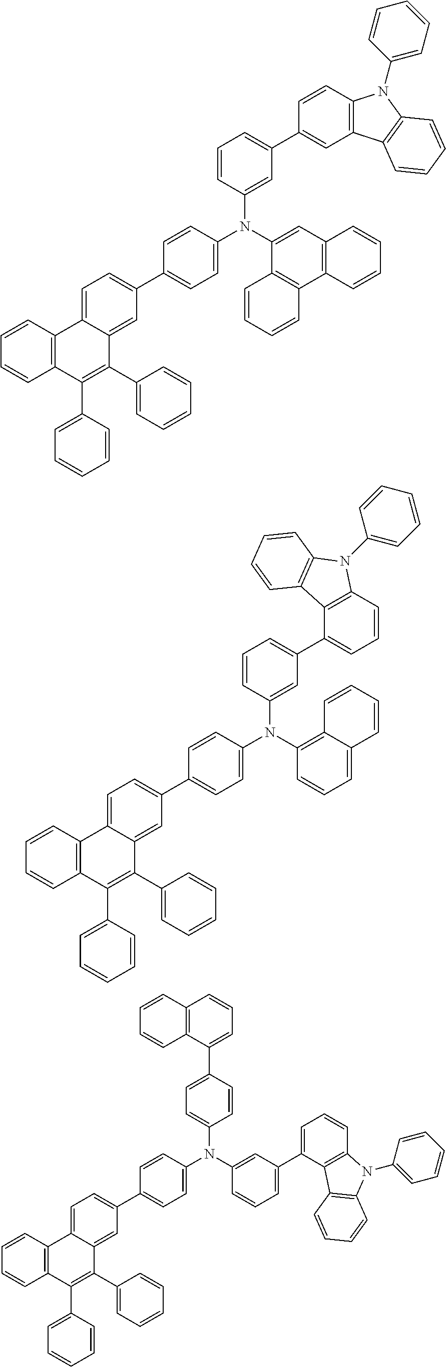

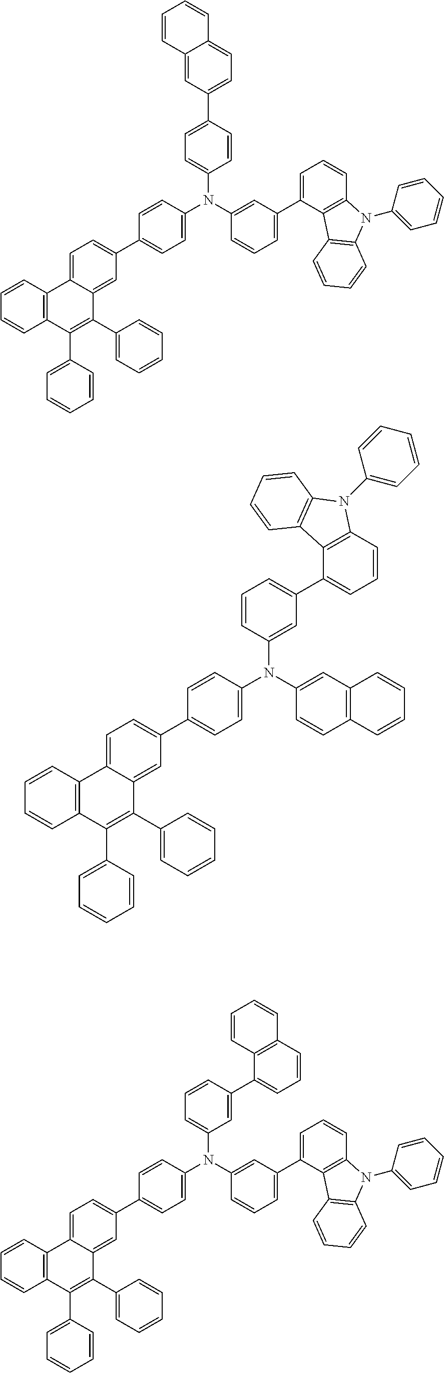 [SMILES: c1ccc(-c2c(-c3ccccc3)c3cc(-c4ccc(N(c5ccc(-c6ccc7ccccc7c6)cc5)c5cccc(-c6cccc7c6c6ccccc6n7-c6ccccc6)c5)cc4)ccc3c3ccccc23)cc1 ; c1ccc(-c2c(-c3ccccc3)c3cc(-c4ccc(N(c5cccc(-c6cccc7c6c6ccccc6n7-c6ccccc6)c5)c5ccc6ccccc6c5)cc4)ccc3c3ccccc23)cc1 ; c1ccc(-c2c(-c3ccccc3)c3cc(-c4ccc(N(c5cccc(-c6cccc7ccccc67)c5)c5cccc(-c6cccc7c6c6ccccc6n7-c6ccccc6)c5)cc4)ccc3c3ccccc23)cc1]

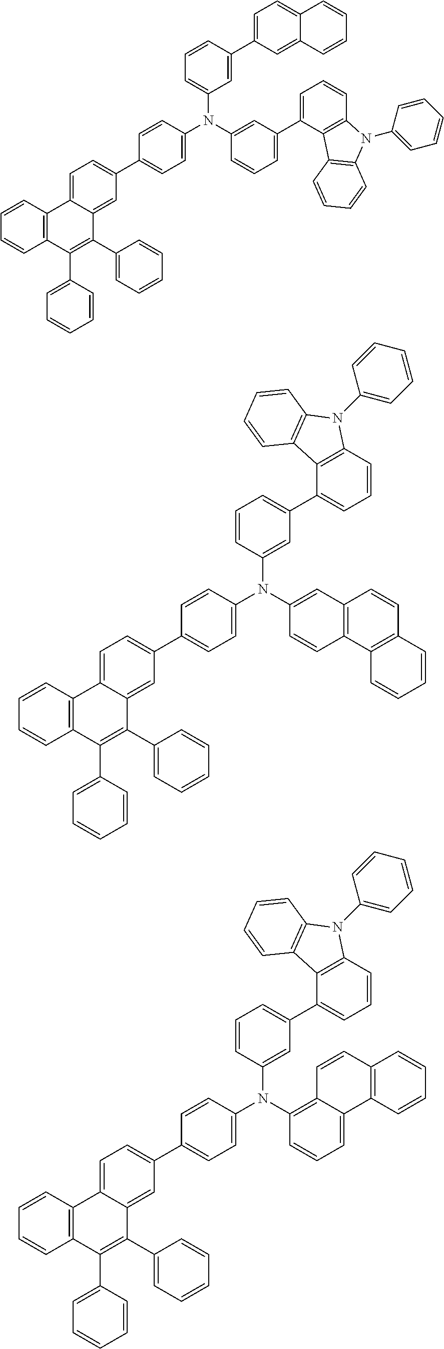 [SMILES: c1ccc(-c2c(-c3ccccc3)c3cc(-c4ccc(N(c5cccc(-c6ccc7ccccc7c6)c5)c5cccc(-c6cccc7c6c6ccccc6n7-c6ccccc6)c5)cc4)ccc3c3ccccc23)cc1 ; c1ccc(-c2c(-c3ccccc3)c3cc(-c4ccc(N(c5cccc(-c6cccc7c6c6ccccc6n7-c6ccccc6)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7c6c6ccccc6n7-c6ccccc6)c5)c5cccc6c5ccc5ccccc56)cc4)ccc3c3ccccc23)cc1]

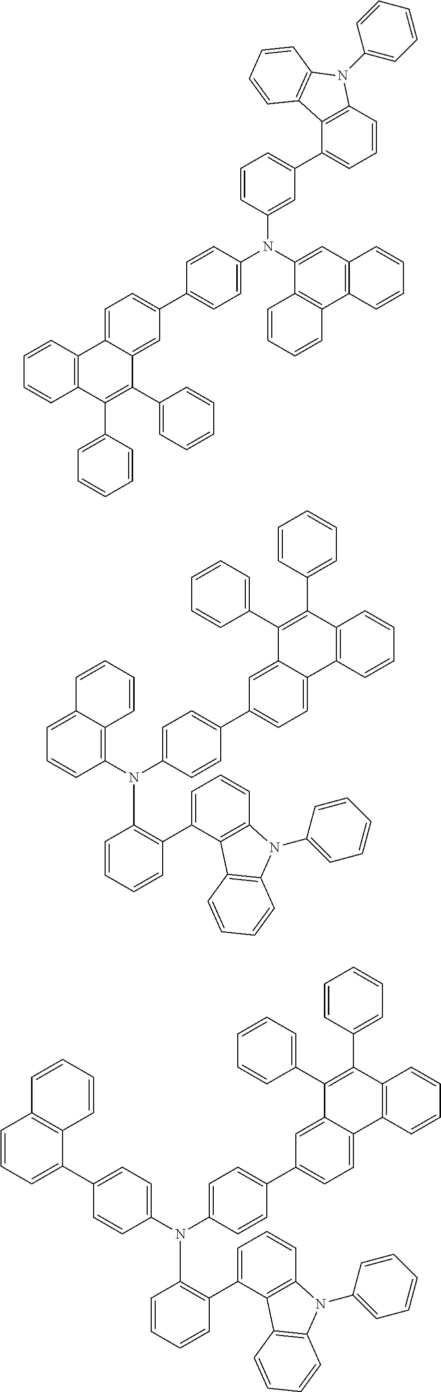 [SMILES: c1ccc(-c2c(-c3ccccc3)c3cc(-c4ccc(N(c5cccc(-c6cccc7c6c6ccccc6n7-c6ccccc6)c5)c5cc6ccccc6c6ccccc56)cc4)ccc3c3ccccc23)cc1 ; c1ccc(-c2c(-c3ccccc3)c3cc(-c4ccc(N(c5ccccc5-c5cccc6c5c5ccccc5n6-c5ccccc5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccccc5-c5cccc6c5c5ccccc5n6-c5ccccc5)cc4)ccc3c3ccccc23)cc1]

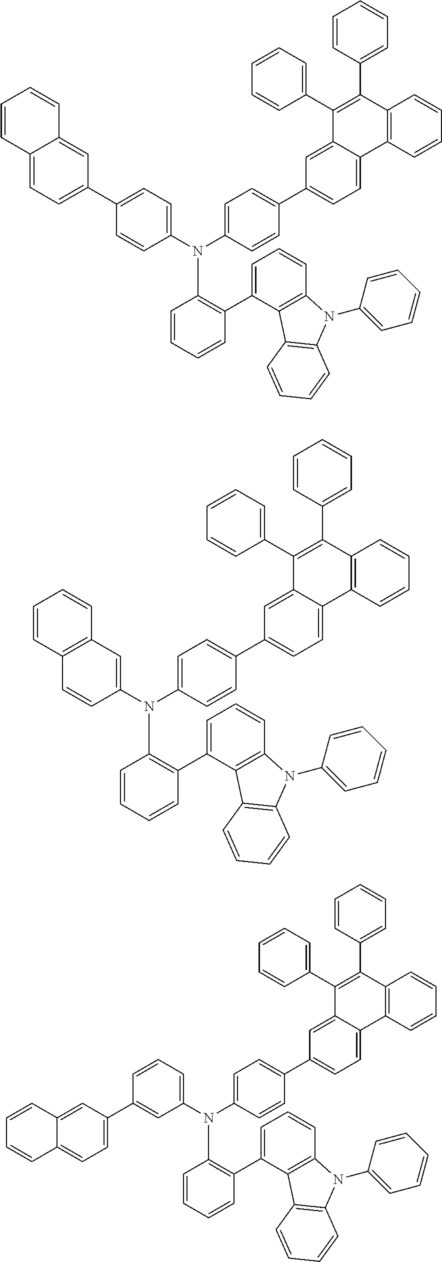

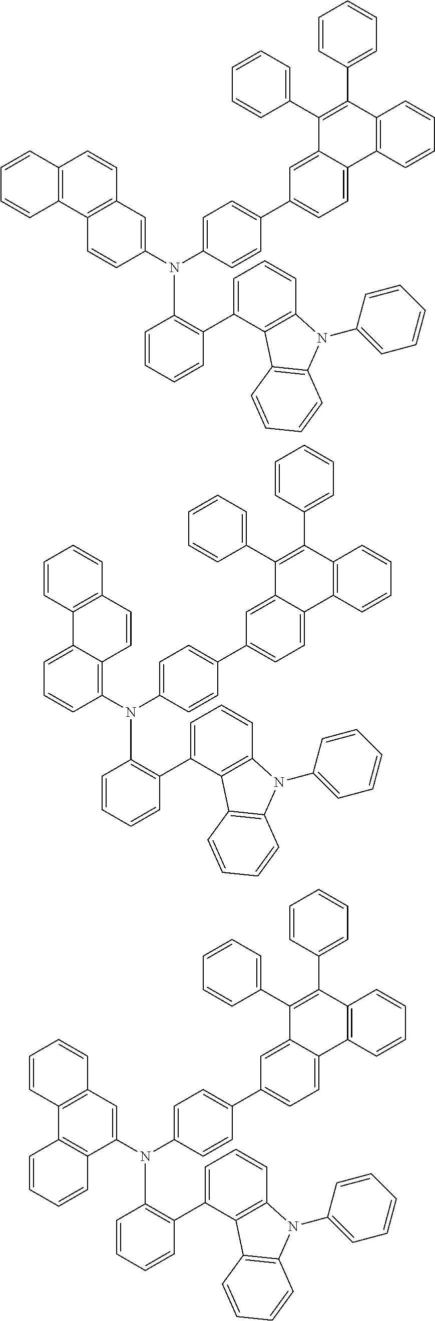

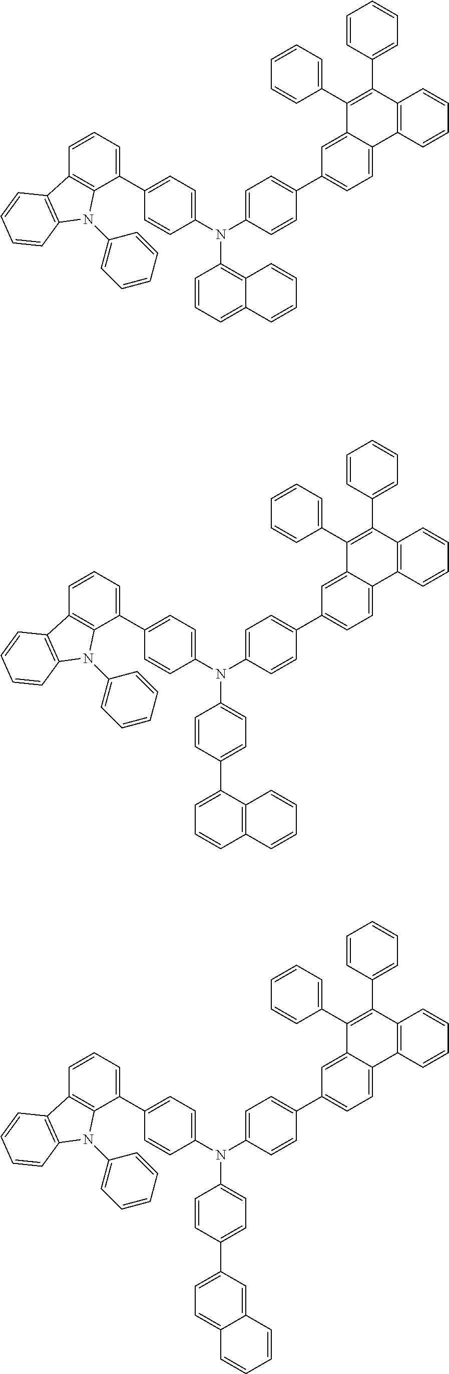 [SMILES: c1ccc(-c2c(-c3ccccc3)c3cc(-c4ccc(N(c5ccc(-c6cccc7c8ccccc8n(-c8ccccc8)c67)cc5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc(-c6cccc7c8ccccc8n(-c8ccccc8)c67)cc5)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6cccc7c8ccccc8n(-c8ccccc8)c67)cc5)cc4)ccc3c3ccccc23)cc1]

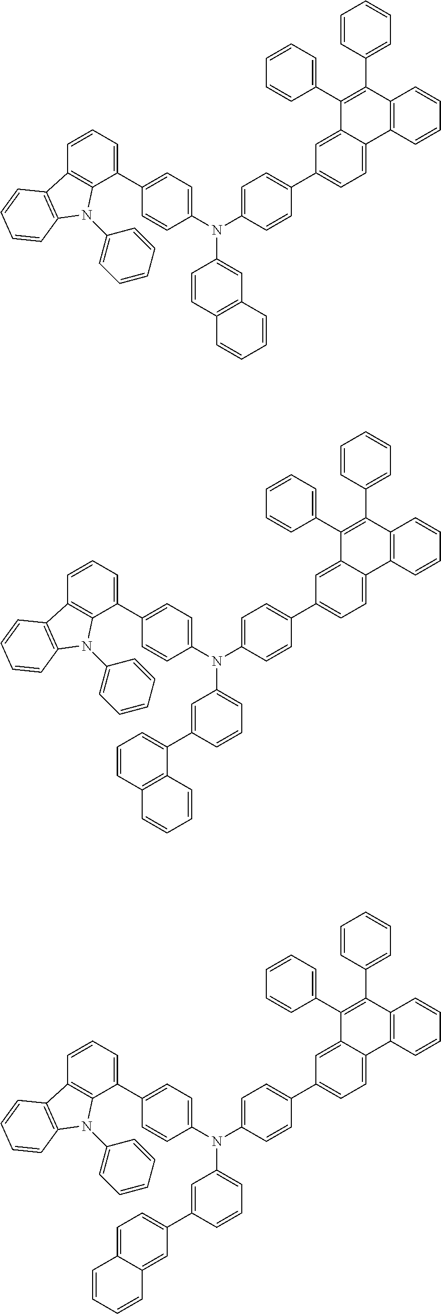 [SMILES: c1ccc(-c2c(-c3ccccc3)c3cc(-c4ccc(N(c5ccc(-c6cccc7c8ccccc8n(-c8ccccc8)c67)cc5)c5ccc6ccccc6c5)cc4)ccc3c3ccccc23)cc1 ; c1ccc(-c2c(-c3ccccc3)c3cc(-c4ccc(N(c5ccc(-c6cccc7c8ccccc8n(-c8ccccc8)c67)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6cccc7c8ccccc8n(-c8ccccc8)c67)cc5)c5cccc(-c6ccc7ccccc7c6)c5)cc4)ccc3c3ccccc23)cc1]

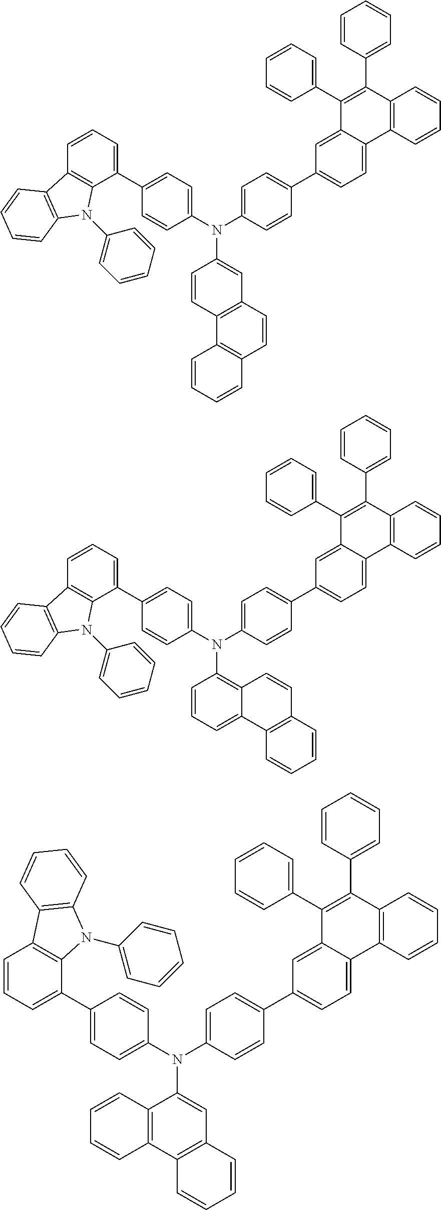 [SMILES: c1ccc(-c2c(-c3ccccc3)c3cc(-c4ccc(N(c5ccc(-c6cccc7c8ccccc8n(-c8ccccc8)c67)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6cccc7c8ccccc8n(-c8ccccc8)c67)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7c8ccccc8n(-c8ccccc8)c67)cc5)c5cc6ccccc6c6ccccc56)cc4)ccc3c3ccccc23)cc1]

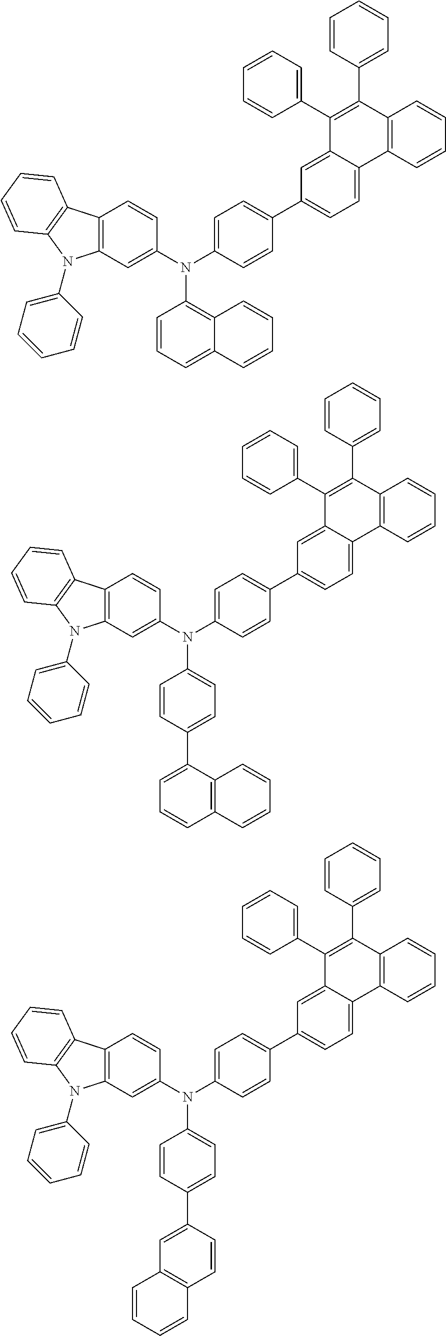 [SMILES: c1ccc(-c2c(-c3ccccc3)c3cc(-c4ccc(N(c5ccc6c7ccccc7n(-c7ccccc7)c6c5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc6c7ccccc7n(-c7ccccc7)c6c5)cc4)ccc3c3ccccc23)cc1 ; c1ccc(-c2c(-c3ccccc3)c3cc(-c4ccc(N(c5ccc(-c6ccc7ccccc7c6)cc5)c5ccc6c7ccccc7n(-c7ccccc7)c6c5)cc4)ccc3c3ccccc23)cc1]

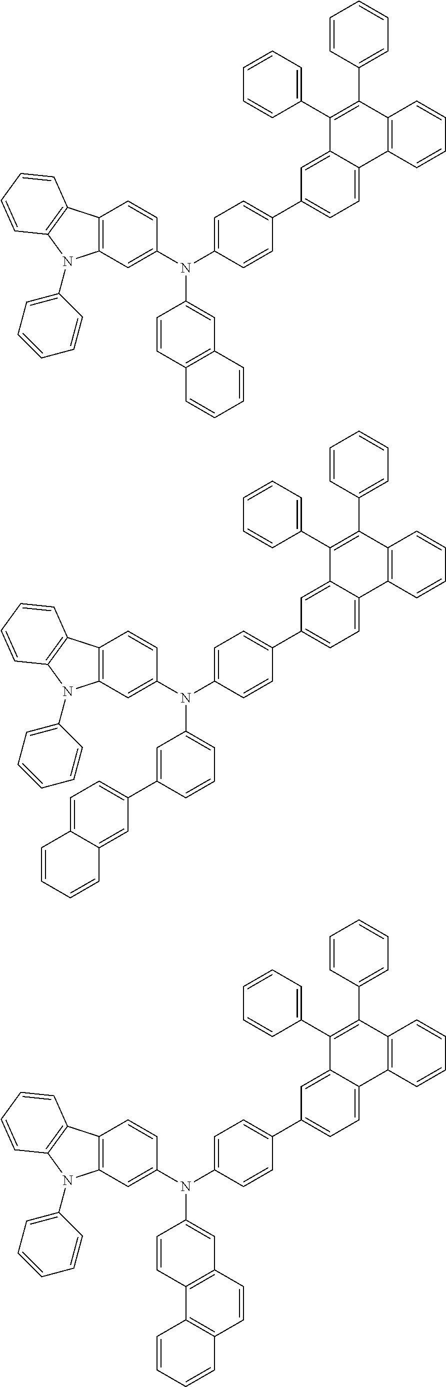 [SMILES: c1ccc(-c2c(-c3ccccc3)c3cc(-c4ccc(N(c5ccc6ccccc6c5)c5ccc6c7ccccc7n(-c7ccccc7)c6c5)cc4)ccc3c3ccccc23)cc1 ; c1ccc(-c2c(-c3ccccc3)c3cc(-c4ccc(N(c5cccc(-c6ccc7ccccc7c6)c5)c5ccc6c7ccccc7n(-c7ccccc7)c6c5)cc4)ccc3c3ccccc23)cc1 ; c1ccc(-c2c(-c3ccccc3)c3cc(-c4ccc(N(c5ccc6c(ccc7ccccc76)c5)c5ccc6c7ccccc7n(-c7ccccc7)c6c5)cc4)ccc3c3ccccc23)cc1]

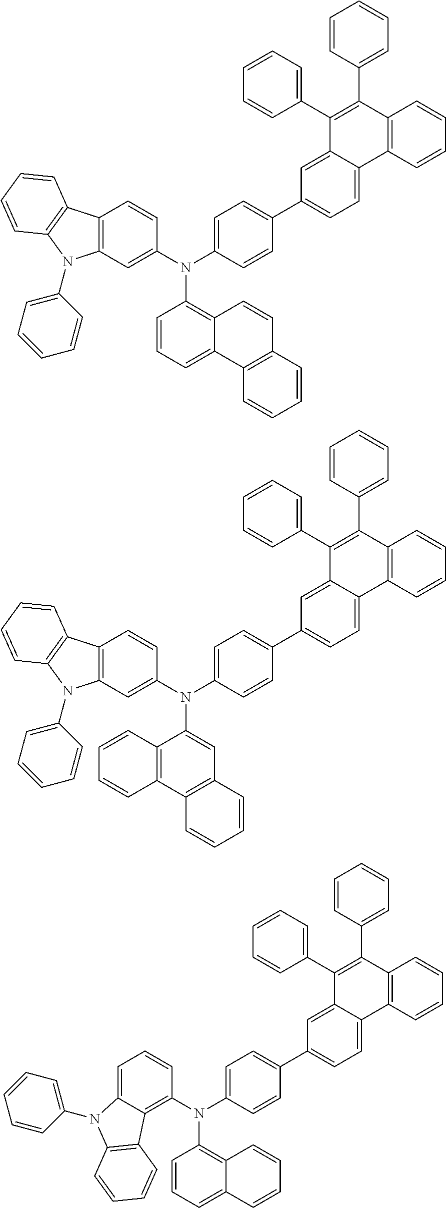 [SMILES: c1ccc(-c2c(-c3ccccc3)c3cc(-c4ccc(N(c5ccc6c7ccccc7n(-c7ccccc7)c6c5)c5cccc6c5ccc5ccccc56)cc4)ccc3c3ccccc23)cc1 ; c1ccc(-c2c(-c3ccccc3)c3cc(-c4ccc(N(c5ccc6c7ccccc7n(-c7ccccc7)c6c5)c5cc6ccccc6c6ccccc56)cc4)ccc3c3ccccc23)cc1 ; c1ccc(-c2c(-c3ccccc3)c3cc(-c4ccc(N(c5cccc6ccccc56)c5cccc6c5c5ccccc5n6-c5ccccc5)cc4)ccc3c3ccccc23)cc1]

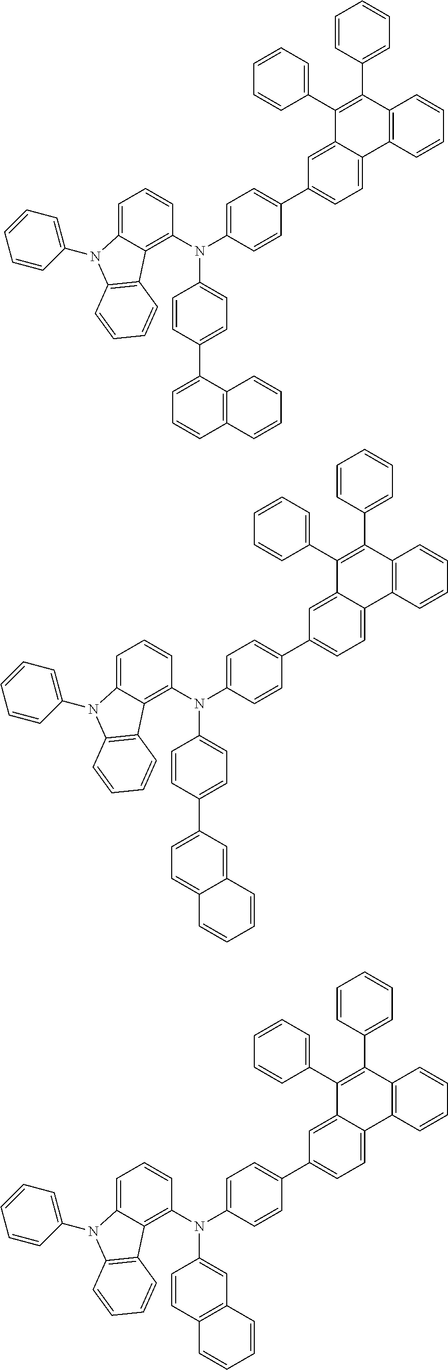 [SMILES: c1ccc(-c2c(-c3ccccc3)c3cc(-c4ccc(N(c5ccc(-c6cccc7ccccc67)cc5)c5cccc6c5c5ccccc5n6-c5ccccc5)cc4)ccc3c3ccccc23)cc1 ; c1ccc(-c2c(-c3ccccc3)c3cc(-c4ccc(N(c5ccc(-c6ccc7ccccc7c6)cc5)c5cccc6c5c5ccccc5n6-c5ccccc5)cc4)ccc3c3ccccc23)cc1 ; c1ccc(-c2c(-c3ccccc3)c3cc(-c4ccc(N(c5ccc6ccccc6c5)c5cccc6c5c5ccccc5n6-c5ccccc5)cc4)ccc3c3ccccc23)cc1]

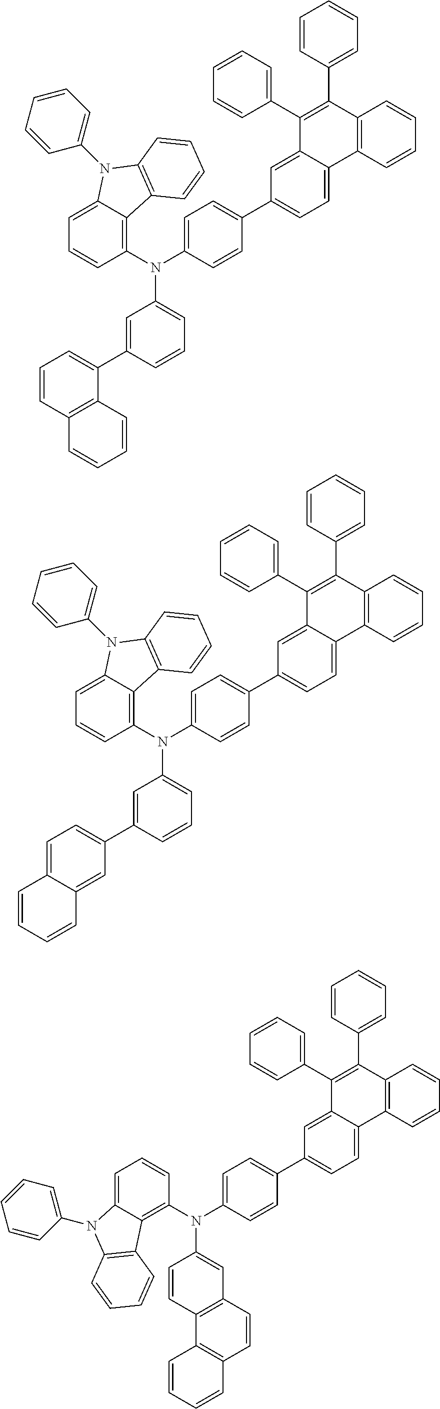 [SMILES: c1ccc(-c2c(-c3ccccc3)c3cc(-c4ccc(N(c5cccc(-c6cccc7ccccc67)c5)c5cccc6c5c5ccccc5n6-c5ccccc5)cc4)ccc3c3ccccc23)cc1 ; c1ccc(-c2c(-c3ccccc3)c3cc(-c4ccc(N(c5cccc(-c6ccc7ccccc7c6)c5)c5cccc6c5c5ccccc5n6-c5ccccc5)cc4)ccc3c3ccccc23)cc1 ; c1ccc(-c2c(-c3ccccc3)c3cc(-c4ccc(N(c5ccc6c(ccc7ccccc76)c5)c5cccc6c5c5ccccc5n6-c5ccccc5)cc4)ccc3c3ccccc23)cc1]

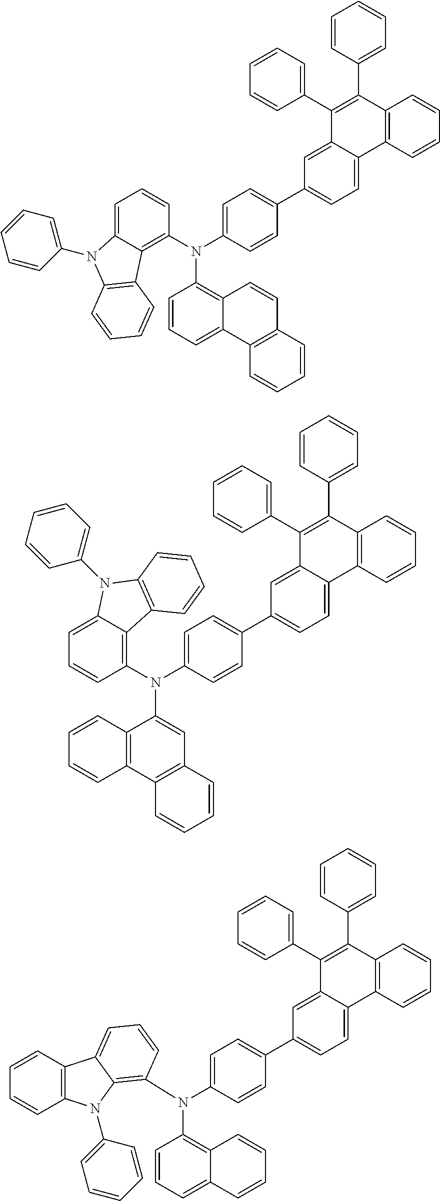 [SMILES: c1ccc(-c2c(-c3ccccc3)c3cc(-c4ccc(N(c5cccc6c5ccc5ccccc56)c5cccc6c5c5ccccc5n6-c5ccccc5)cc4)ccc3c3ccccc23)cc1 ; c1ccc(-c2c(-c3ccccc3)c3cc(-c4ccc(N(c5cc6ccccc6c6ccccc56)c5cccc6c5c5ccccc5n6-c5ccccc5)cc4)ccc3c3ccccc23)cc1 ; c1ccc(-c2c(-c3ccccc3)c3cc(-c4ccc(N(c5cccc6ccccc56)c5cccc6c7ccccc7n(-c7ccccc7)c56)cc4)ccc3c3ccccc23)cc1]

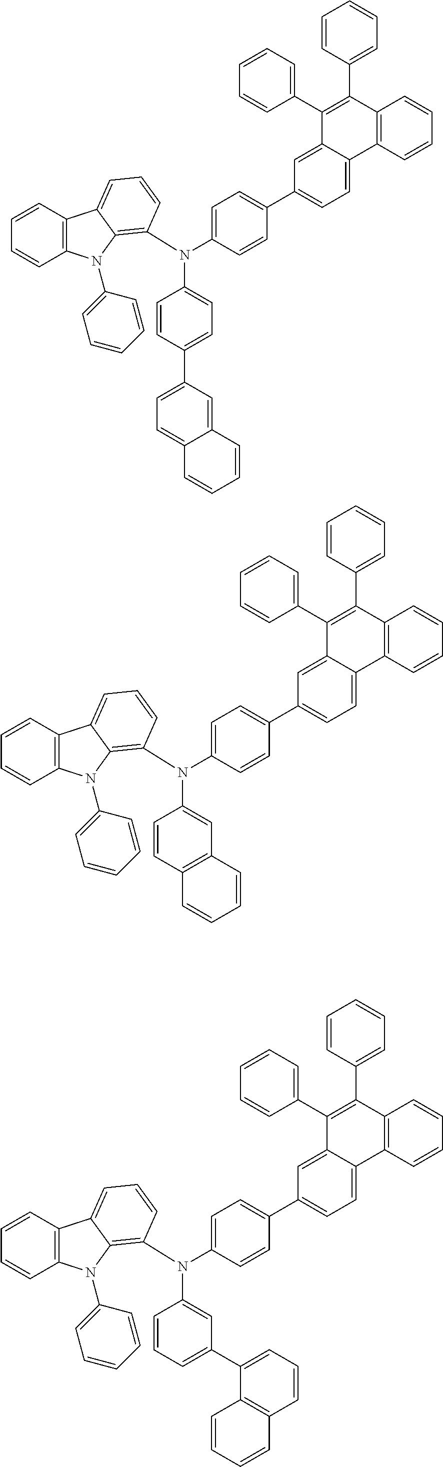 [SMILES: c1ccc(-c2c(-c3ccccc3)c3cc(-c4ccc(N(c5ccc(-c6ccc7ccccc7c6)cc5)c5cccc6c7ccccc7n(-c7ccccc7)c56)cc4)ccc3c3ccccc23)cc1 ; c1ccc(-c2c(-c3ccccc3)c3cc(-c4ccc(N(c5ccc6ccccc6c5)c5cccc6c7ccccc7n(-c7ccccc7)c56)cc4)ccc3c3ccccc23)cc1 ; c1ccc(-c2c(-c3ccccc3)c3cc(-c4ccc(N(c5cccc(-c6cccc7ccccc67)c5)c5cccc6c7ccccc7n(-c7ccccc7)c56)cc4)ccc3c3ccccc23)cc1]

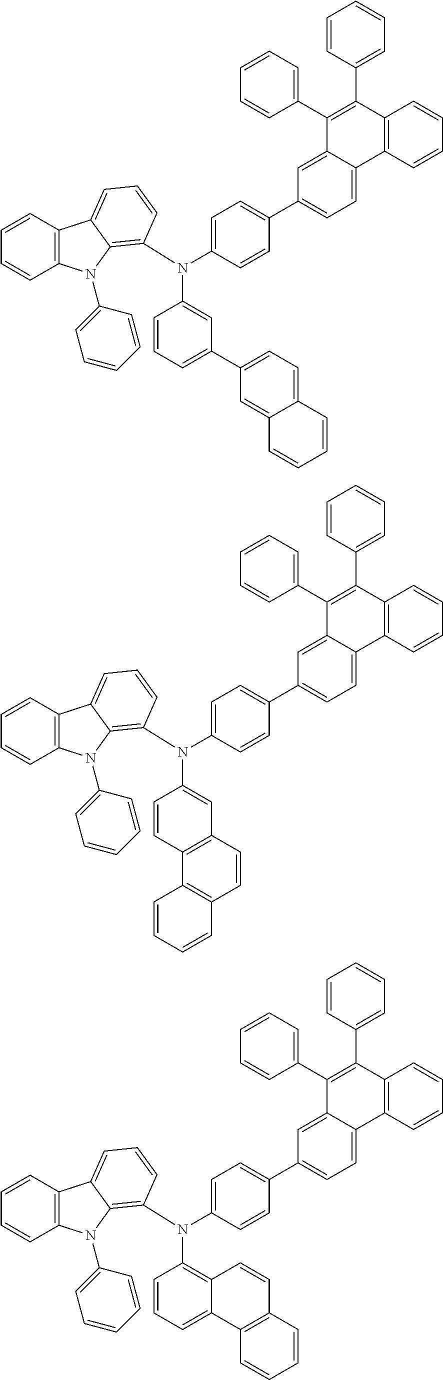

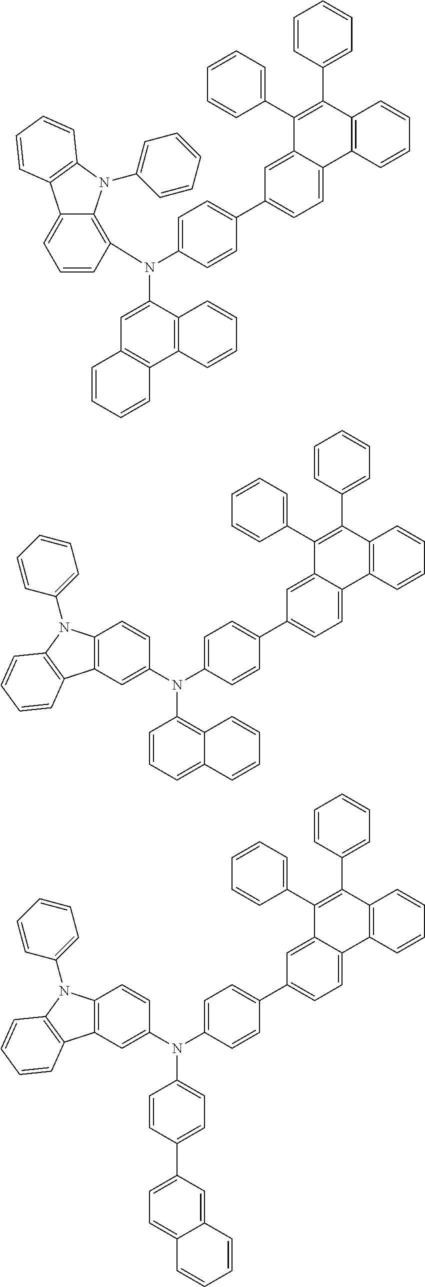 [SMILES: c1ccc(-c2c(-c3ccccc3)c3cc(-c4ccc(N(c5cc6ccccc6c6ccccc56)c5cccc6c7ccccc7n(-c7ccccc7)c56)cc4)ccc3c3ccccc23)cc1 ; c1ccc(-c2c(-c3ccccc3)c3cc(-c4ccc(N(c5ccc6c(c5)c5ccccc5n6-c5ccccc5)c5cccc6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5ccc6c(c5)c5ccccc5n6-c5ccccc5)cc4)ccc3c3ccccc23)cc1]

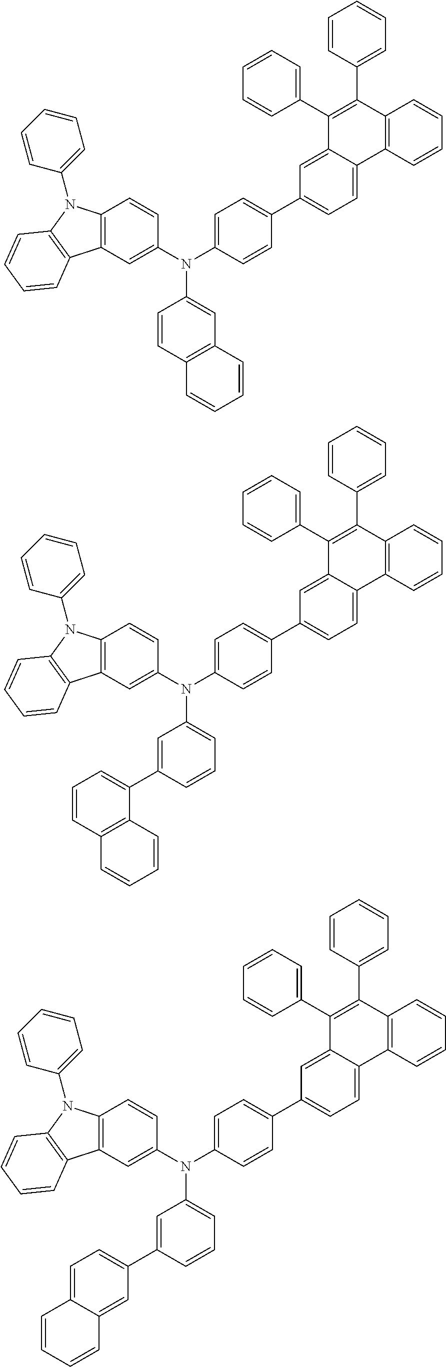 [SMILES: c1ccc(-c2c(-c3ccccc3)c3cc(-c4ccc(N(c5ccc6ccccc6c5)c5ccc6c(c5)c5ccccc5n6-c5ccccc5)cc4)ccc3c3ccccc23)cc1 ; c1ccc(-c2c(-c3ccccc3)c3cc(-c4ccc(N(c5cccc(-c6cccc7ccccc67)c5)c5ccc6c(c5)c5ccccc5n6-c5ccccc5)cc4)ccc3c3ccccc23)cc1 ; c1ccc(-c2c(-c3ccccc3)c3cc(-c4ccc(N(c5cccc(-c6ccc7ccccc7c6)c5)c5ccc6c(c5)c5ccccc5n6-c5ccccc5)cc4)ccc3c3ccccc23)cc1]

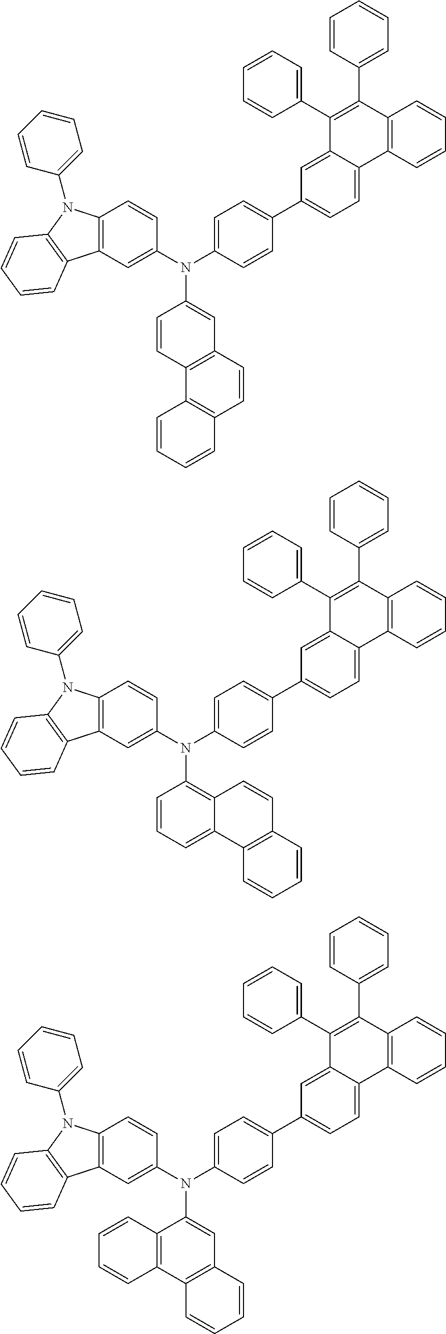 [SMILES: c1ccc(-c2c(-c3ccccc3)c3cc(-c4ccc(N(c5ccc6c(ccc7ccccc76)c5)c5ccc6c(c5)c5ccccc5n6-c5ccccc5)cc4)ccc3c3ccccc23)cc1 ; c1ccc(-c2c(-c3ccccc3)c3cc(-c4ccc(N(c5ccc6c(c5)c5ccccc5n6-c5ccccc5)c5cccc6c5ccc5ccccc56)cc4)ccc3c3ccccc23)cc1 ; c1ccc(-c2c(-c3ccccc3)c3cc(-c4ccc(N(c5ccc6c(c5)c5ccccc5n6-c5ccccc5)c5cc6ccccc6c6ccccc56)cc4)ccc3c3ccccc23)cc1]

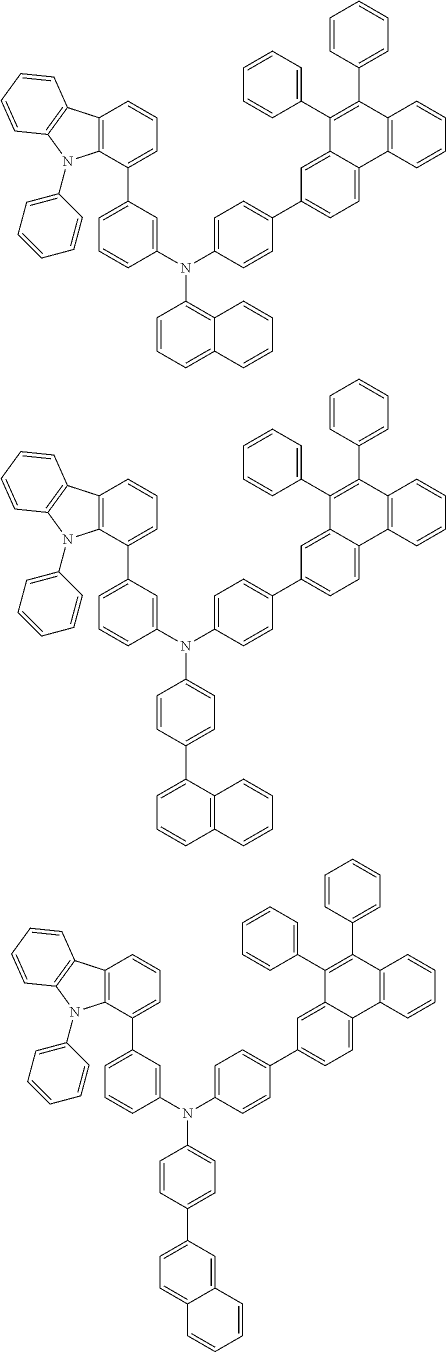 [SMILES: c1ccc(-c2c(-c3ccccc3)c3cc(-c4ccc(N(c5cccc(-c6cccc7c8ccccc8n(-c8ccccc8)c67)c5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5cccc(-c6cccc7c8ccccc8n(-c8ccccc8)c67)c5)cc4)ccc3c3ccccc23)cc1 ; c1ccc(-c2c(-c3ccccc3)c3cc(-c4ccc(N(c5ccc(-c6ccc7ccccc7c6)cc5)c5cccc(-c6cccc7c8ccccc8n(-c8ccccc8)c67)c5)cc4)ccc3c3ccccc23)cc1]

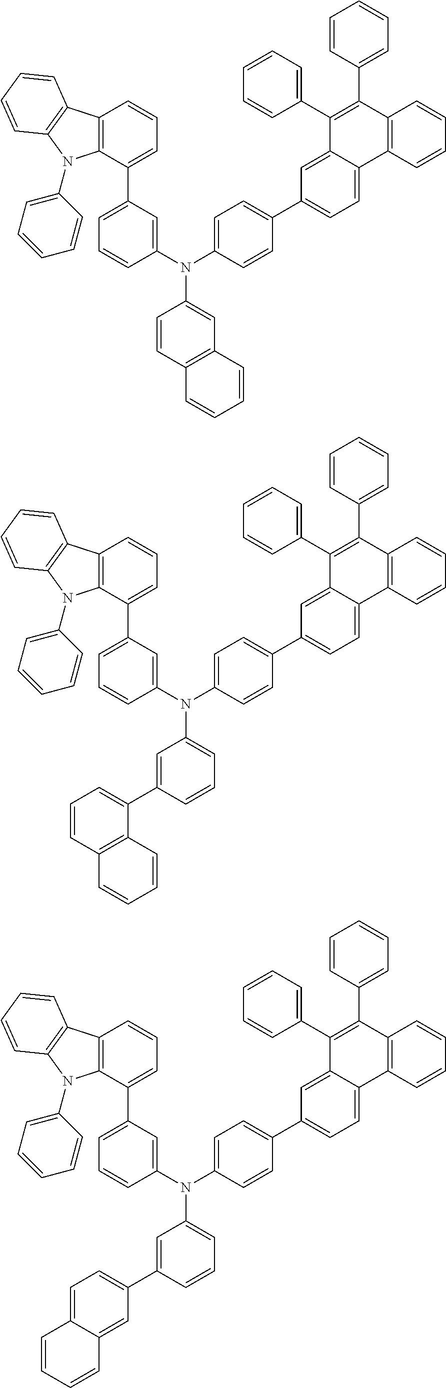 [SMILES: c1ccc(-c2c(-c3ccccc3)c3cc(-c4ccc(N(c5cccc(-c6cccc7c8ccccc8n(-c8ccccc8)c67)c5)c5ccc6ccccc6c5)cc4)ccc3c3ccccc23)cc1 ; c1ccc(-c2c(-c3ccccc3)c3cc(-c4ccc(N(c5cccc(-c6cccc7ccccc67)c5)c5cccc(-c6cccc7c8ccccc8n(-c8ccccc8)c67)c5)cc4)ccc3c3ccccc23)cc1 ; c1ccc(-c2c(-c3ccccc3)c3cc(-c4ccc(N(c5cccc(-c6ccc7ccccc7c6)c5)c5cccc(-c6cccc7c8ccccc8n(-c8ccccc8)c67)c5)cc4)ccc3c3ccccc23)cc1]

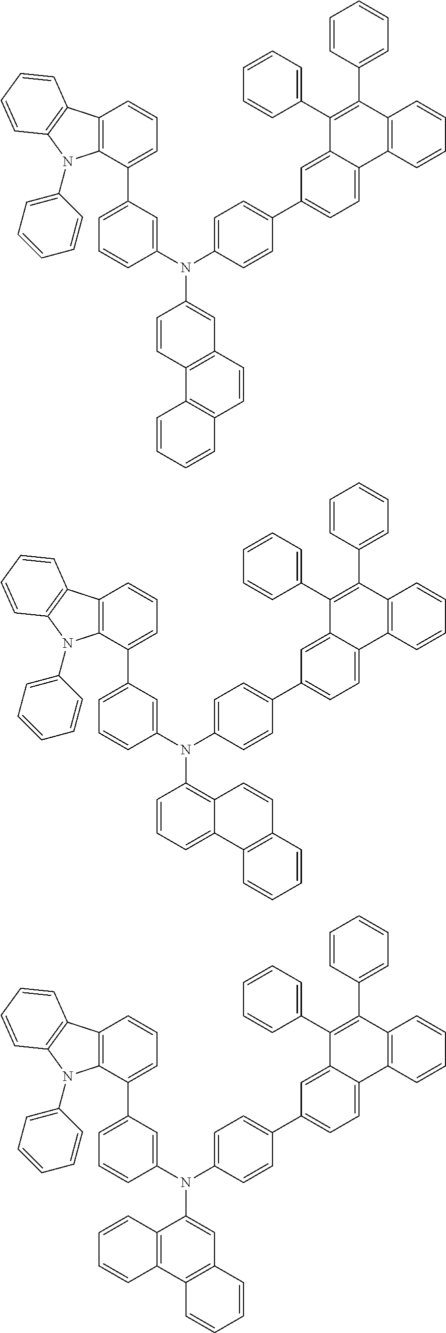 [SMILES: c1ccc(-c2c(-c3ccccc3)c3cc(-c4ccc(N(c5cccc(-c6cccc7c8ccccc8n(-c8ccccc8)c67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7c8ccccc8n(-c8ccccc8)c67)c5)c5cccc6c5ccc5ccccc56)cc4)ccc3c3ccccc23)cc1 ; c1ccc(-c2c(-c3ccccc3)c3cc(-c4ccc(N(c5cccc(-c6cccc7c8ccccc8n(-c8ccccc8)c67)c5)c5cc6ccccc6c6ccccc56)cc4)ccc3c3ccccc23)cc1]

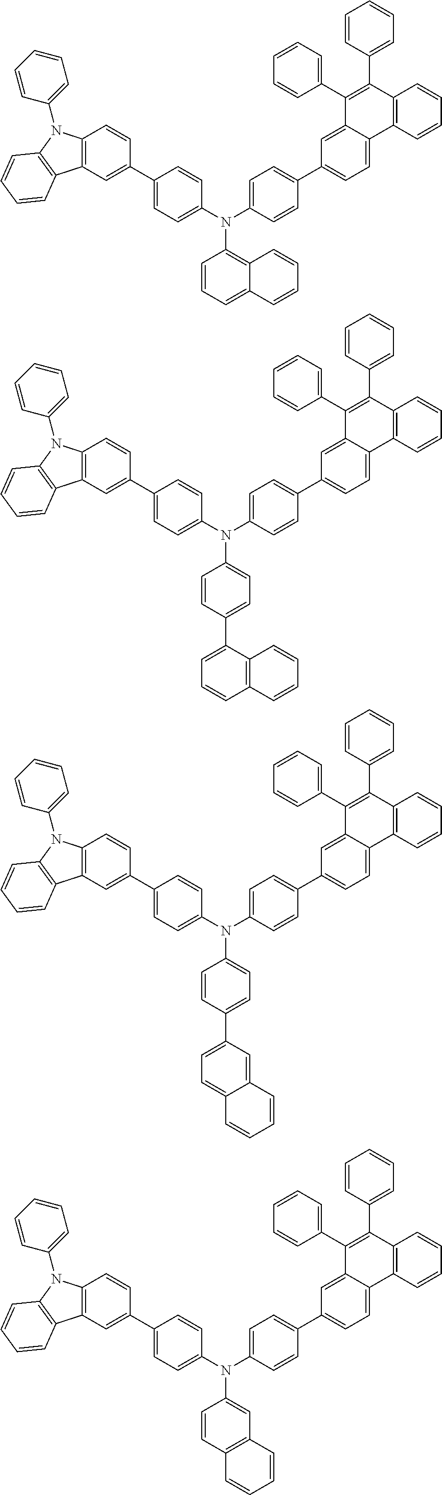 [SMILES: c1ccc(-c2c(-c3ccccc3)c3cc(-c4ccc(N(c5ccc(-c6ccc7c(c6)c6ccccc6n7-c6ccccc6)cc5)c5cccc6ccccc56)cc4)ccc3c3ccccc23)cc1 ; c1ccc(-c2c(-c3ccccc3)c3cc(-c4ccc(N(c5ccc(-c6ccc7c(c6)c6ccccc6n7-c6ccccc6)cc5)c5ccc(-c6cccc7ccccc67)cc5)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6ccc7c(c6)c6ccccc6n7-c6ccccc6)cc5)cc4)ccc3c3ccccc23)cc1 ; c1ccc(-c2c(-c3ccccc3)c3cc(-c4ccc(N(c5ccc(-c6ccc7c(c6)c6ccccc6n7-c6ccccc6)cc5)c5ccc6ccccc6c5)cc4)ccc3c3ccccc23)cc1]

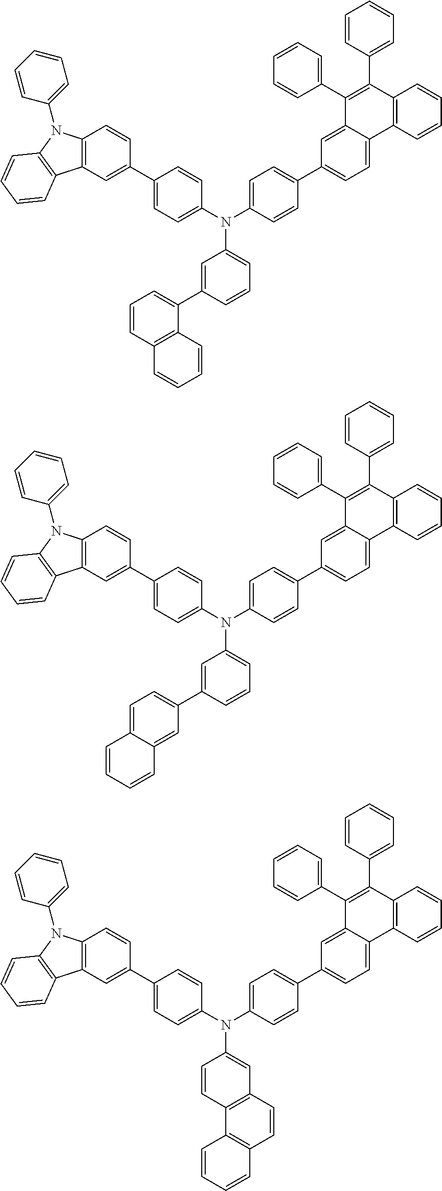 [SMILES: c1ccc(-c2c(-c3ccccc3)c3cc(-c4ccc(N(c5ccc(-c6ccc7c(c6)c6ccccc6n7-c6ccccc6)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6ccc7c(c6)c6ccccc6n7-c6ccccc6)cc5)c5cccc(-c6ccc7ccccc7c6)c5)cc4)ccc3c3ccccc23)cc1 ; c1ccc(-c2c(-c3ccccc3)c3cc(-c4ccc(N(c5ccc(-c6ccc7c(c6)c6ccccc6n7-c6ccccc6)cc5)c5ccc6c(ccc7ccccc76)c5)cc4)ccc3c3ccccc23)cc1]

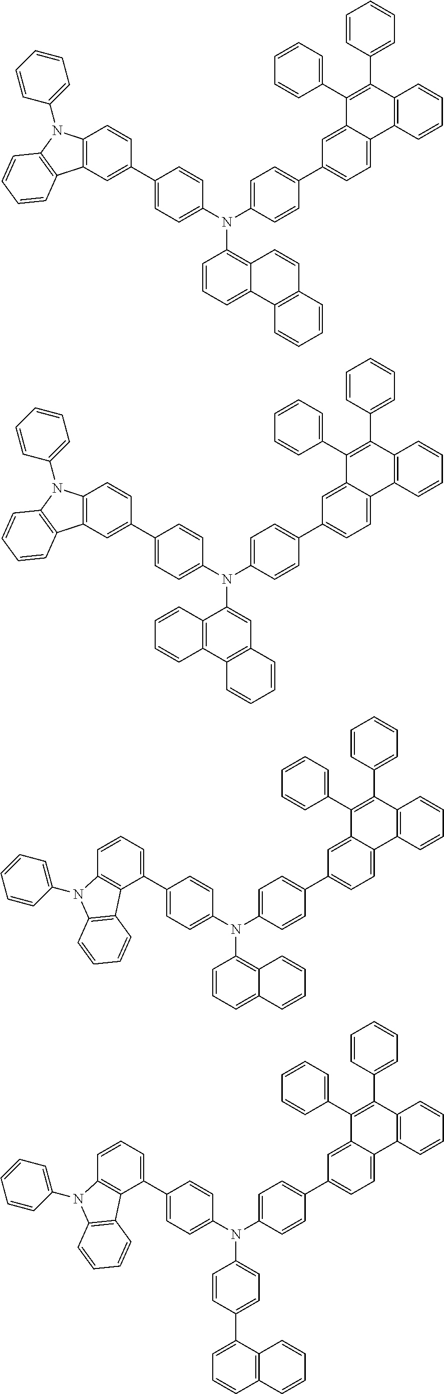 [SMILES: c1ccc(-c2c(-c3ccccc3)c3cc(-c4ccc(N(c5ccc(-c6ccc7c(c6)c6ccccc6n7-c6ccccc6)cc5)c5cccc6c5ccc5ccccc56)cc4)ccc3c3ccccc23)cc1 ; c1ccc(-c2c(-c3ccccc3)c3cc(-c4ccc(N(c5ccc(-c6ccc7c(c6)c6ccccc6n7-c6ccccc6)cc5)c5cc6ccccc6c6ccccc56)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc(-c6cccc7c6c6ccccc6n7-c6ccccc6)cc5)cc4)ccc3c3ccccc23)cc1]

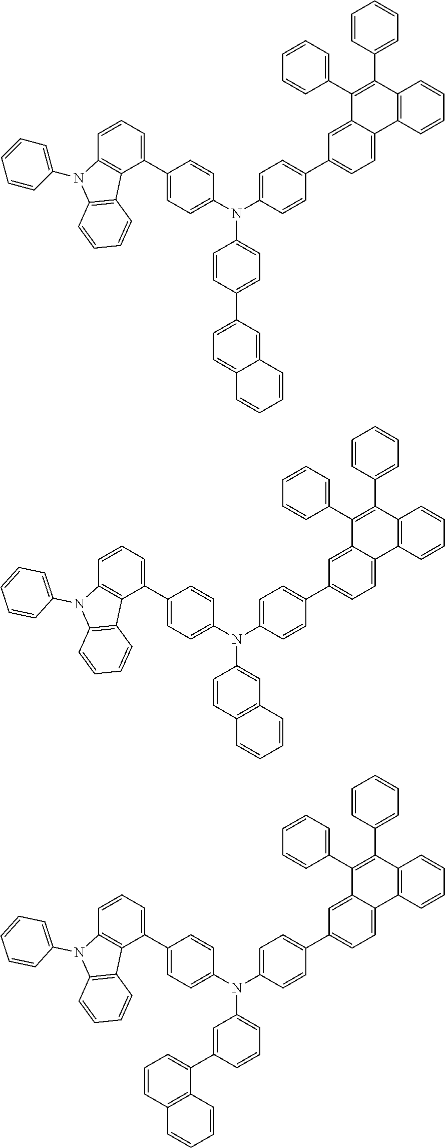 [SMILES: c1ccc(-c2c(-c3ccccc3)c3cc(-c4ccc(N(c5ccc(-c6ccc7ccccc7c6)cc5)c5ccc(-c6cccc7c6c6ccccc6n7-c6ccccc6)cc5)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5ccc6ccccc6c5)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5cccc(-c6cccc7ccccc67)c5)cc4)ccc3c3ccccc23)cc1]

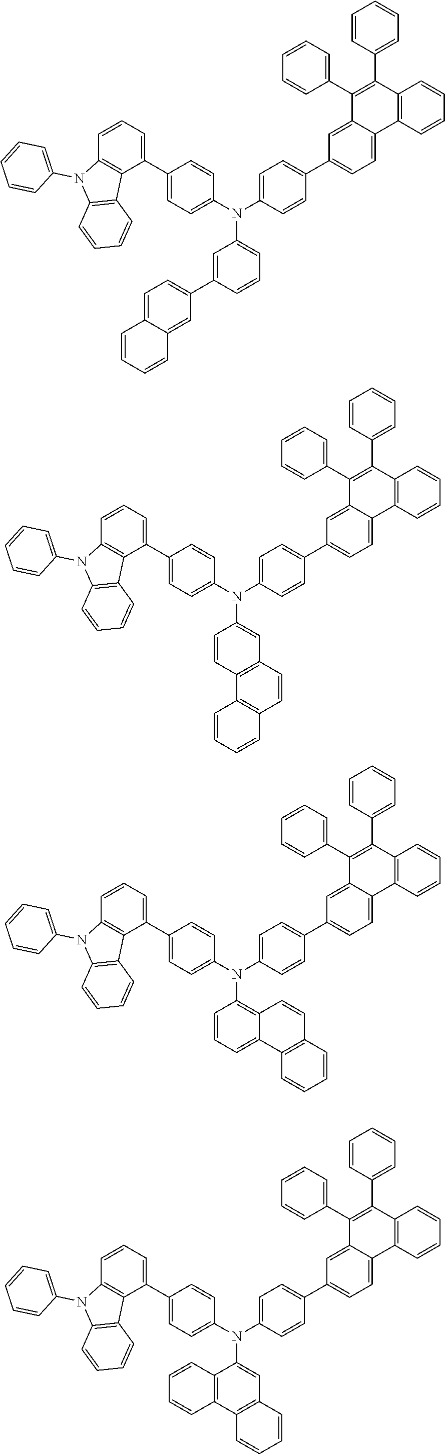 [SMILES: c1ccc(-c2c(-c3ccccc3)c3cc(-c4ccc(N(c5ccc(-c6cccc7c6c6ccccc6n7-c6ccccc6)cc5)c5cccc(-c6ccc7ccccc7c6)c5)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7c6c6ccccc6n7-c6ccccc6)cc5)c5cc6ccccc6c6ccccc56)cc4)ccc3c3ccccc23)cc1]

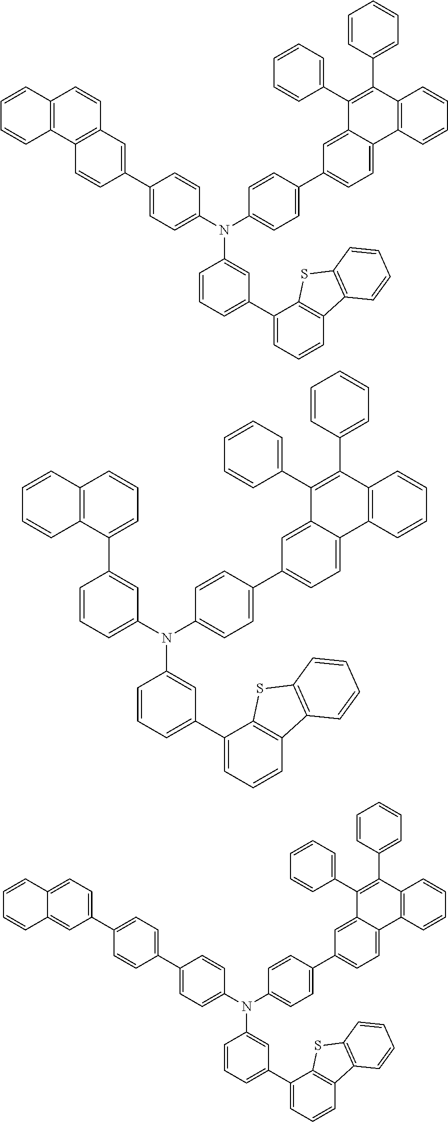

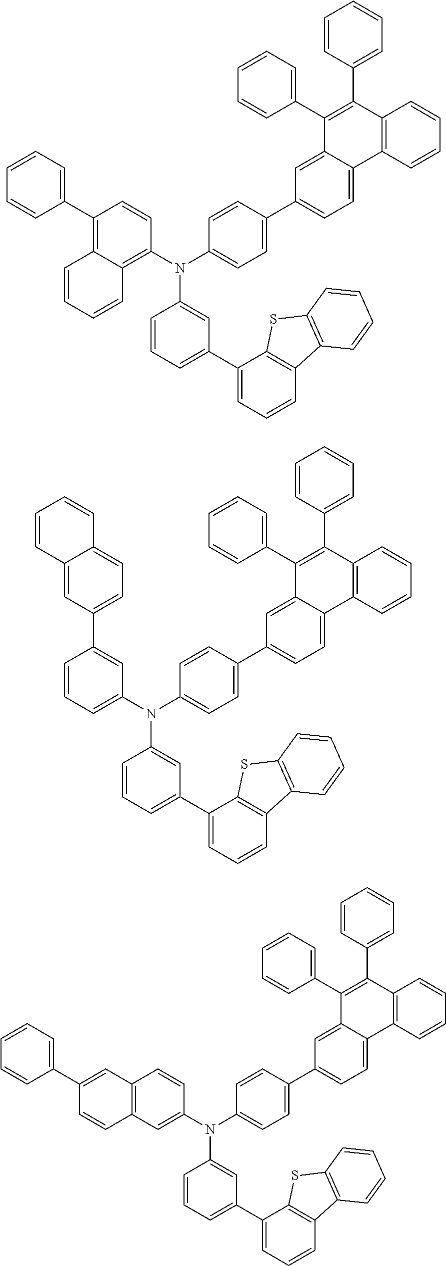 [SMILES: c1ccc(-c2ccc(N(c3ccc(-c4ccc5c(c4)c(-c4ccccc4)c(-c4ccccc4)c4ccccc45)cc3)c3cccc(-c4cccc5c4sc4ccccc45)c3)c3ccccc23)cc1 ; c1ccc(-c2c(-c3ccccc3)c3cc(-c4ccc(N(c5cccc(-c6ccc7ccccc7c6)c5)c5cccc(-c6cccc7c6sc6ccccc67)c5)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4cccc(-c5cccc6c5sc5ccccc56)c4)ccc3c2)cc1]

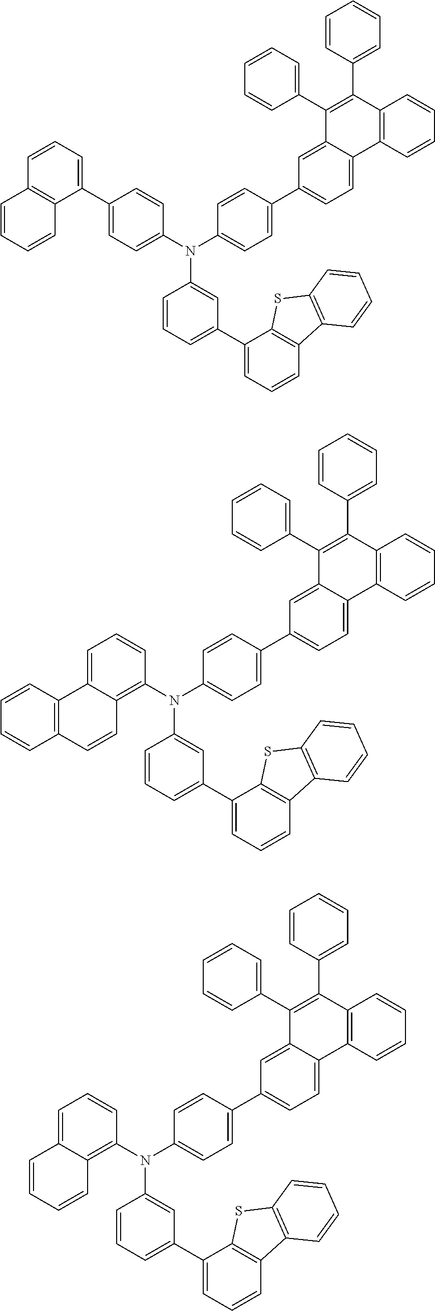 [SMILES: c1ccc(-c2c(-c3ccccc3)c3cc(-c4ccc(N(c5ccc(-c6cccc7ccccc67)cc5)c5cccc(-c6cccc7c6sc6ccccc67)c5)cc4)ccc3c3ccccc23)cc1 ; c1ccc(-c2c(-c3ccccc3)c3cc(-c4ccc(N(c5cccc(-c6cccc7c6sc6ccccc67)c5)c5cccc6c5ccc5ccccc56)cc4)ccc3c3ccccc23)cc1 ; c1ccc(-c2c(-c3ccccc3)c3cc(-c4ccc(N(c5cccc(-c6cccc7c6sc6ccccc67)c5)c5cccc6ccccc56)cc4)ccc3c3ccccc23)cc1]

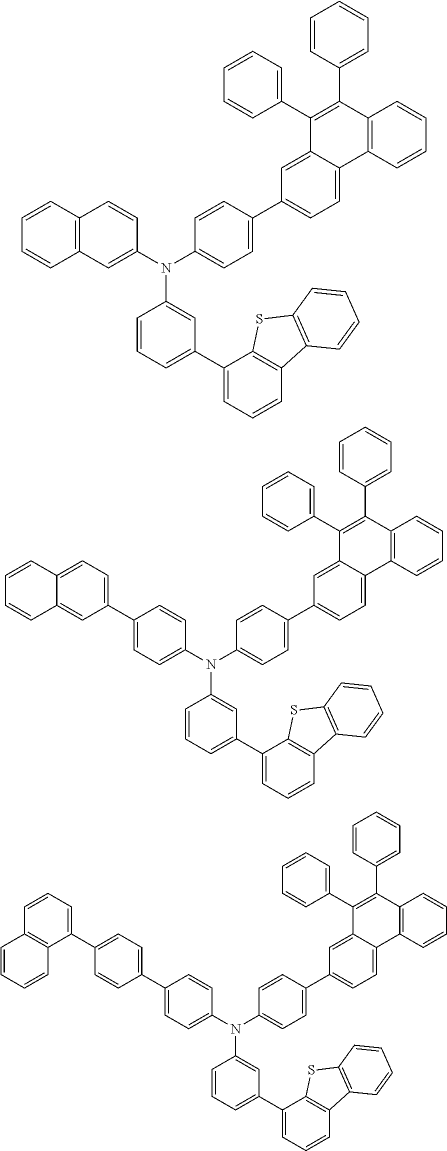 [SMILES: c1ccc(-c2c(-c3ccccc3)c3cc(-c4ccc(N(c5cccc(-c6cccc7c6sc6ccccc67)c5)c5ccc6ccccc6c5)cc4)ccc3c3ccccc23)cc1 ; c1ccc(-c2c(-c3ccccc3)c3cc(-c4ccc(N(c5ccc(-c6ccc7ccccc7c6)cc5)c5cccc(-c6cccc7c6sc6ccccc67)c5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cccc(-c6cccc7c6sc6ccccc67)c5)cc4)ccc3c3ccccc23)cc1]

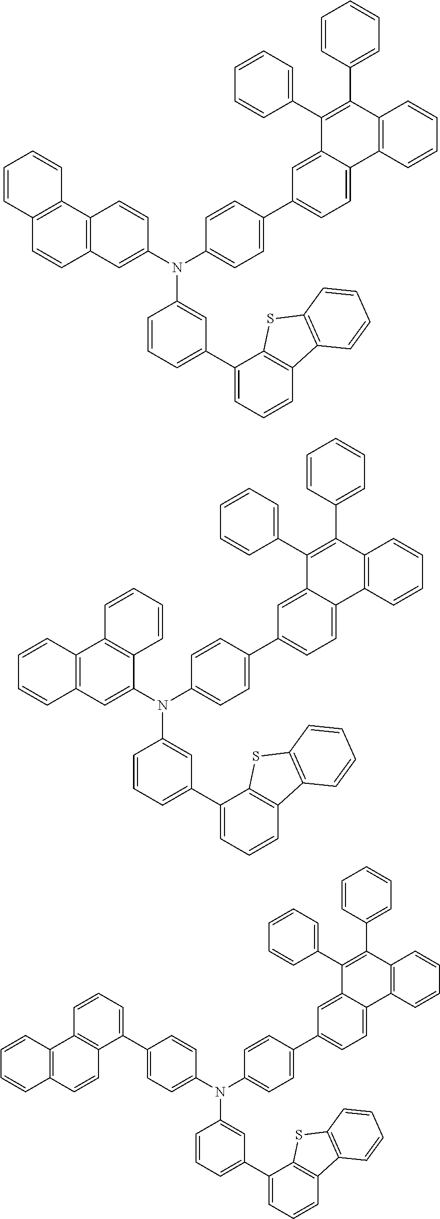 [SMILES: c1ccc(-c2c(-c3ccccc3)c3cc(-c4ccc(N(c5cccc(-c6cccc7c6sc6ccccc67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7c6sc6ccccc67)c5)c5cc6ccccc6c6ccccc56)cc4)ccc3c3ccccc23)cc1 ; c1ccc(-c2c(-c3ccccc3)c3cc(-c4ccc(N(c5ccc(-c6cccc7c6ccc6ccccc67)cc5)c5cccc(-c6cccc7c6sc6ccccc67)c5)cc4)ccc3c3ccccc23)cc1]

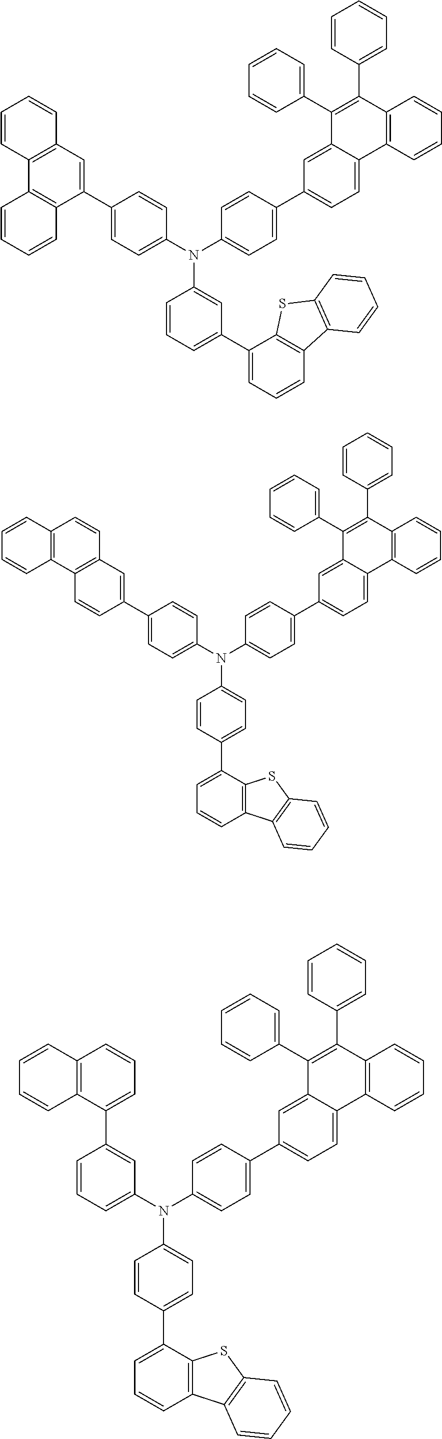 [SMILES: c1ccc(-c2c(-c3ccccc3)c3cc(-c4ccc(N(c5ccc(-c6cc7ccccc7c7ccccc67)cc5)c5cccc(-c6cccc7c6sc6ccccc67)c5)cc4)ccc3c3ccccc23)cc1 ; c1ccc(-c2c(-c3ccccc3)c3cc(-c4ccc(N(c5ccc(-c6ccc7c(ccc8ccccc87)c6)cc5)c5ccc(-c6cccc7c6sc6ccccc67)cc5)cc4)ccc3c3ccccc23)cc1 ; c1ccc(-c2c(-c3ccccc3)c3cc(-c4ccc(N(c5ccc(-c6cccc7c6sc6ccccc67)cc5)c5cccc(-c6cccc7ccccc67)c5)cc4)ccc3c3ccccc23)cc1]

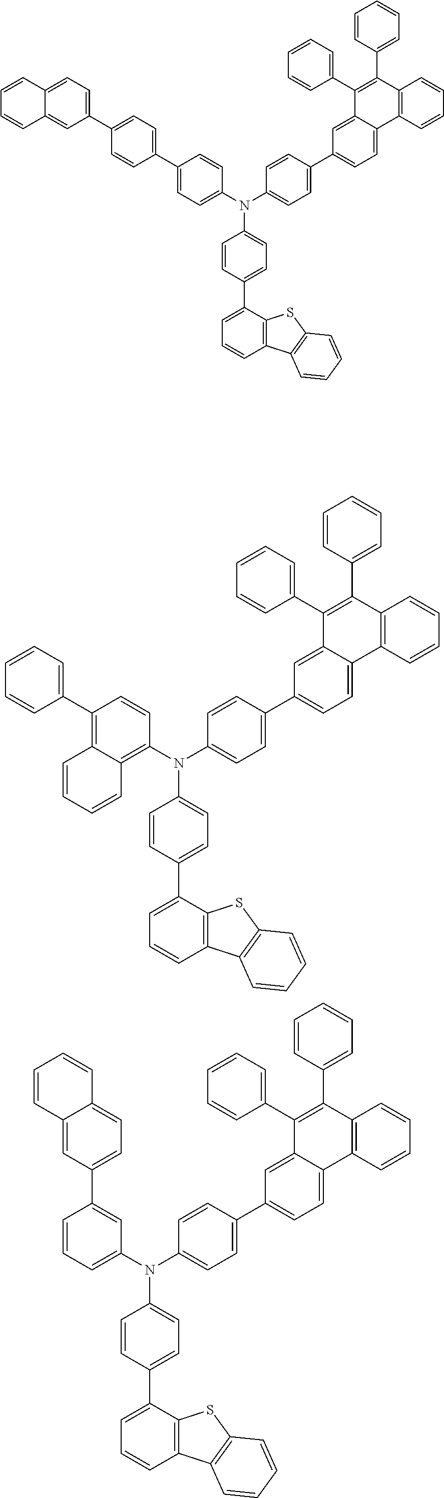

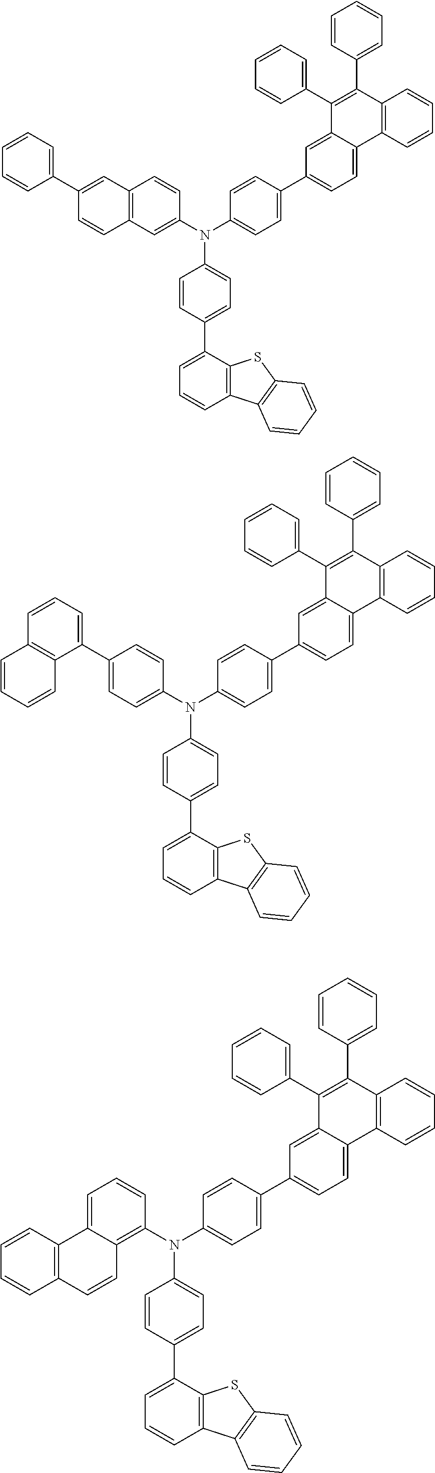 [SMILES: c1ccc(-c2ccc3cc(N(c4ccc(-c5ccc6c(c5)c(-c5ccccc5)c(-c5ccccc5)c5ccccc56)cc4)c4ccc(-c5cccc6c5sc5ccccc56)cc4)ccc3c2)cc1 ; c1ccc(-c2c(-c3ccccc3)c3cc(-c4ccc(N(c5ccc(-c6cccc7ccccc67)cc5)c5ccc(-c6cccc7c6sc6ccccc67)cc5)cc4)ccc3c3ccccc23)cc1 ; c1ccc(-c2c(-c3ccccc3)c3cc(-c4ccc(N(c5ccc(-c6cccc7c6sc6ccccc67)cc5)c5cccc6c5ccc5ccccc56)cc4)ccc3c3ccccc23)cc1]

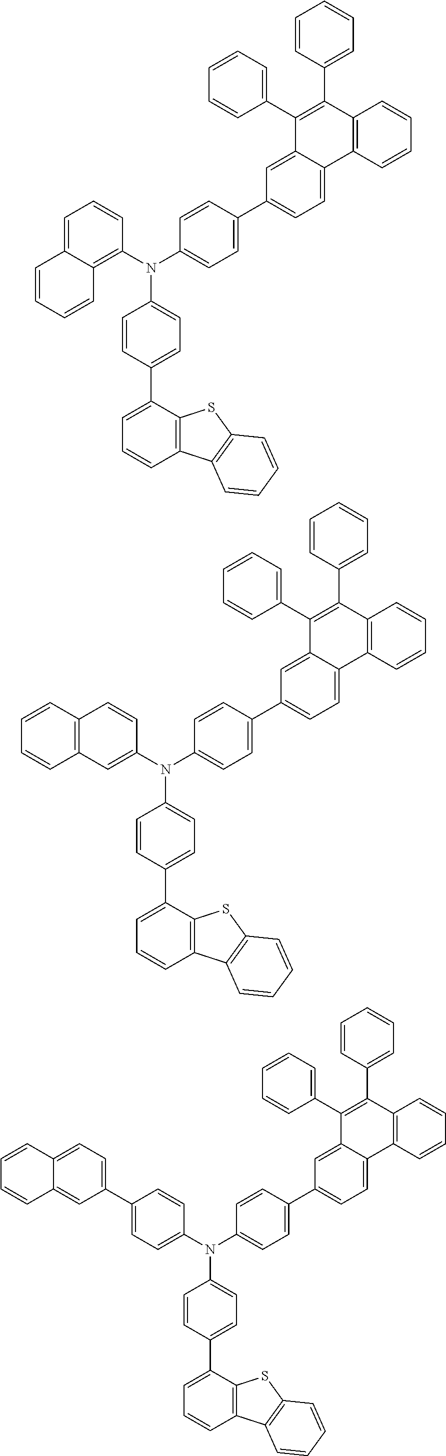 [SMILES: c1ccc(-c2c(-c3ccccc3)c3cc(-c4ccc(N(c5ccc(-c6cccc7c6sc6ccccc67)cc5)c5cccc6ccccc56)cc4)ccc3c3ccccc23)cc1 ; c1ccc(-c2c(-c3ccccc3)c3cc(-c4ccc(N(c5ccc(-c6cccc7c6sc6ccccc67)cc5)c5ccc6ccccc6c5)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6cccc7c6sc6ccccc67)cc5)cc4)ccc3c3ccccc23)cc1]

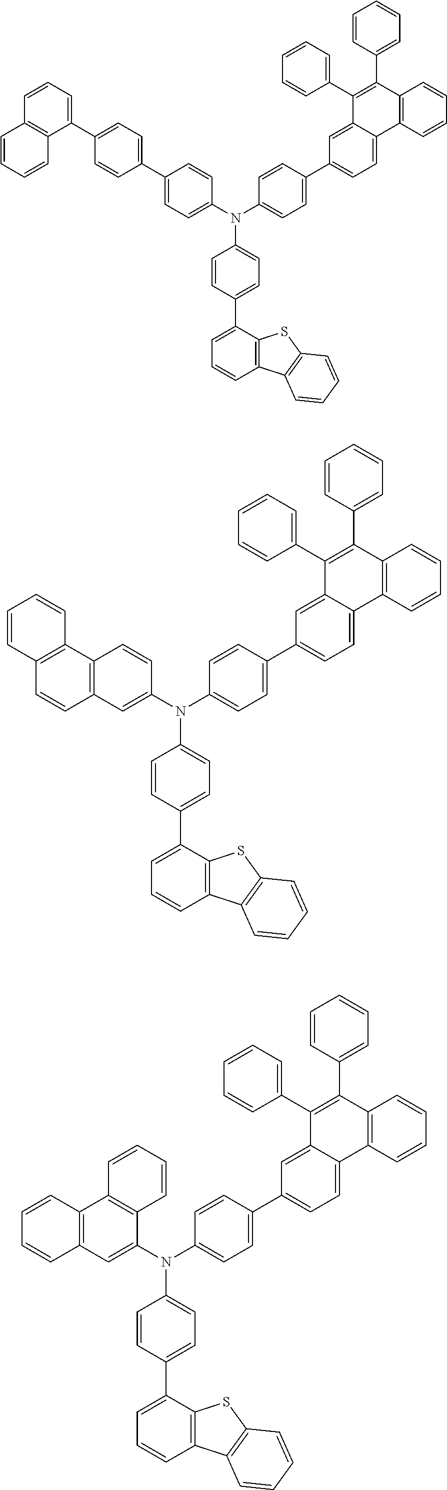 [SMILES: c1ccc(-c2c(-c3ccccc3)c3cc(-c4ccc(N(c5ccc(-c6ccc(-c7cccc8ccccc78)cc6)cc5)c5ccc(-c6cccc7c6sc6ccccc67)cc5)cc4)ccc3c3ccccc23)cc1 ; c1ccc(-c2c(-c3ccccc3)c3cc(-c4ccc(N(c5ccc(-c6cccc7c6sc6ccccc67)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6cccc7c6sc6ccccc67)cc5)c5cc6ccccc6c6ccccc56)cc4)ccc3c3ccccc23)cc1]

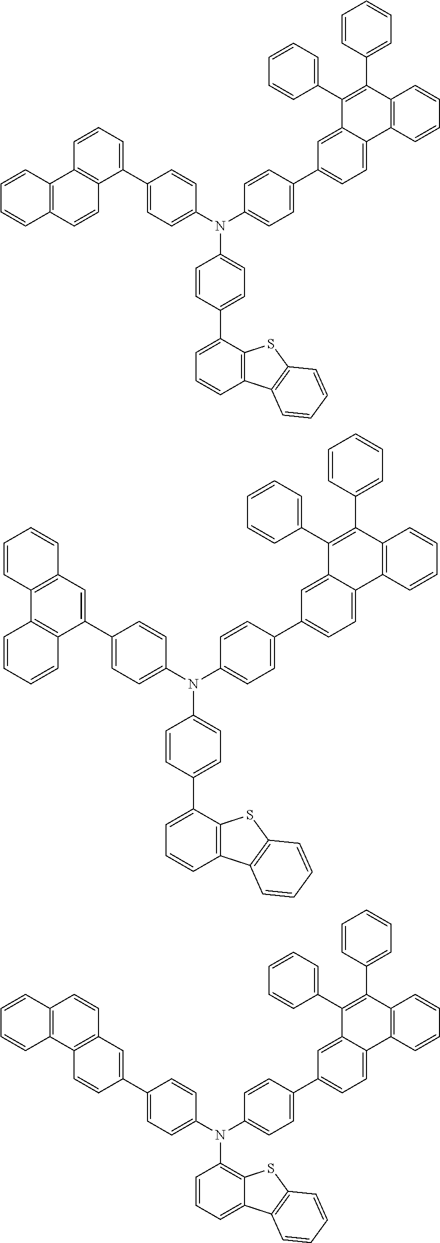

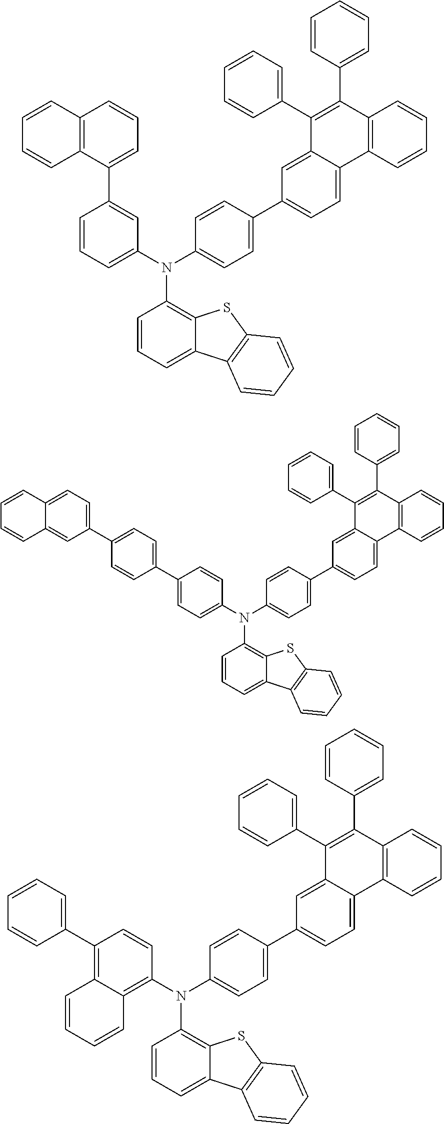

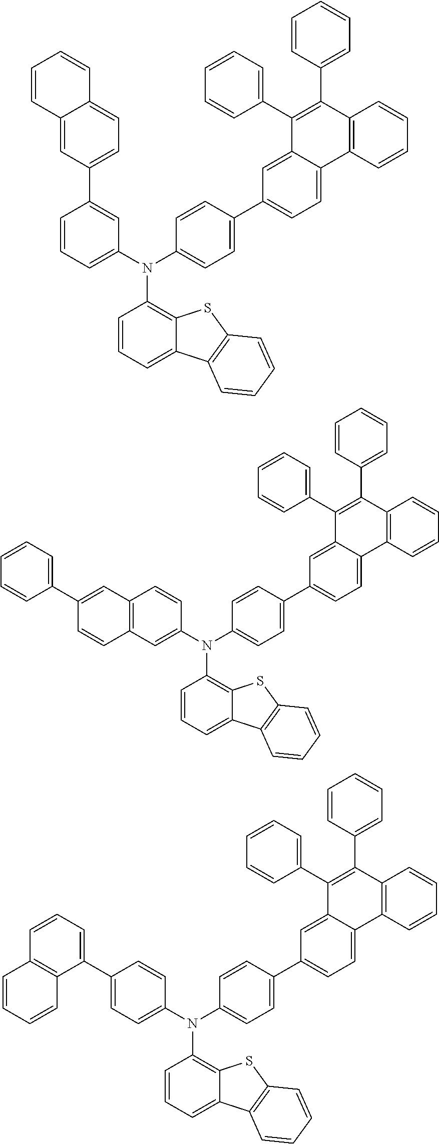

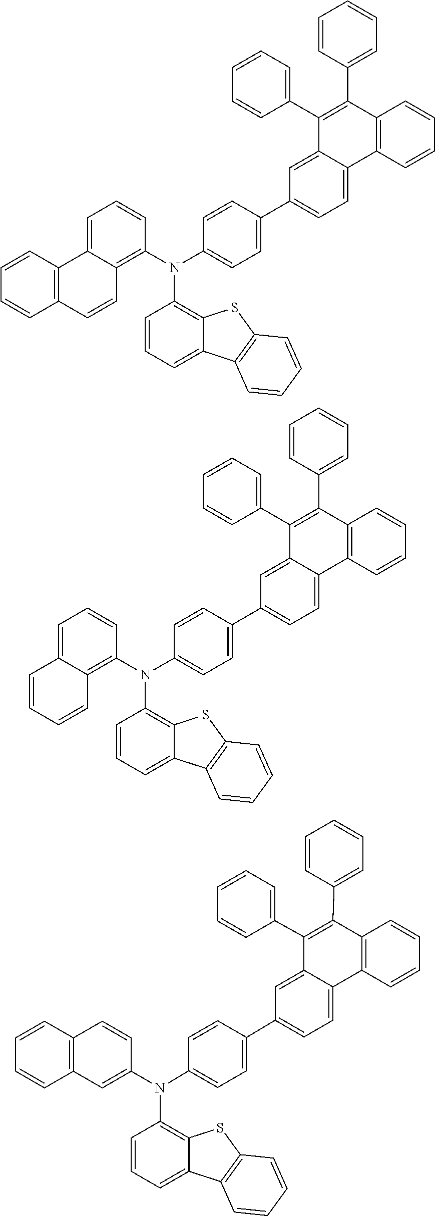

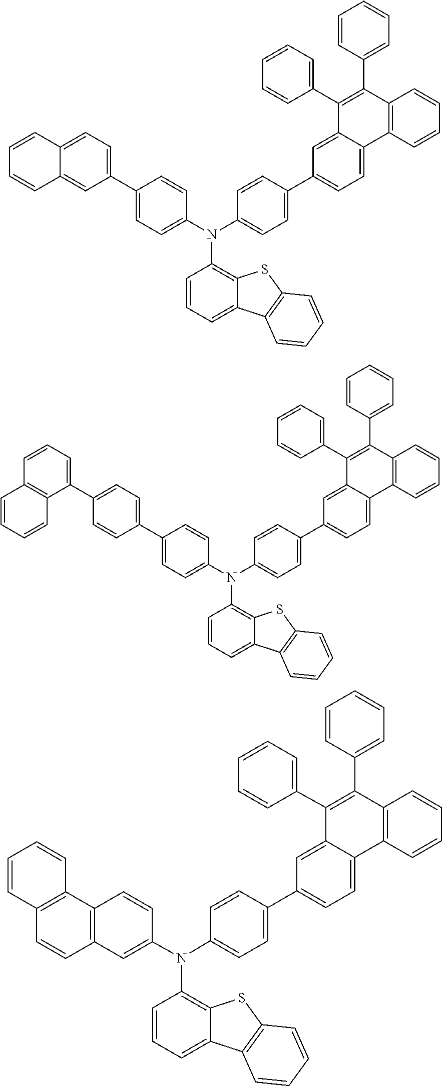

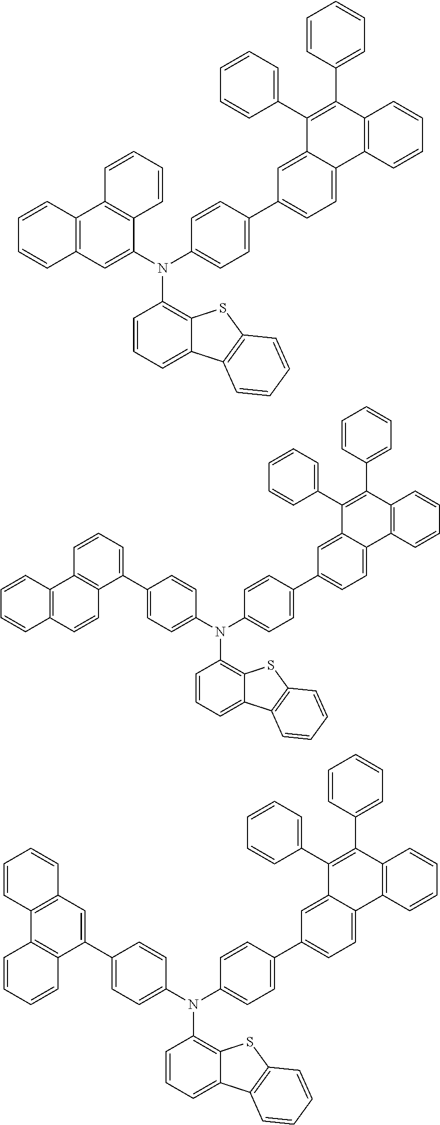

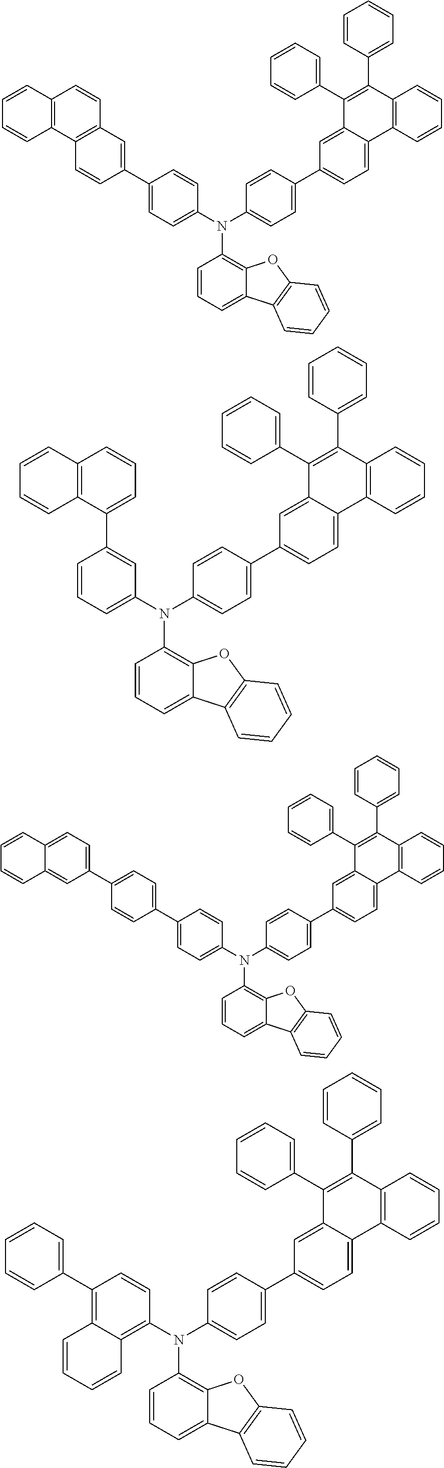

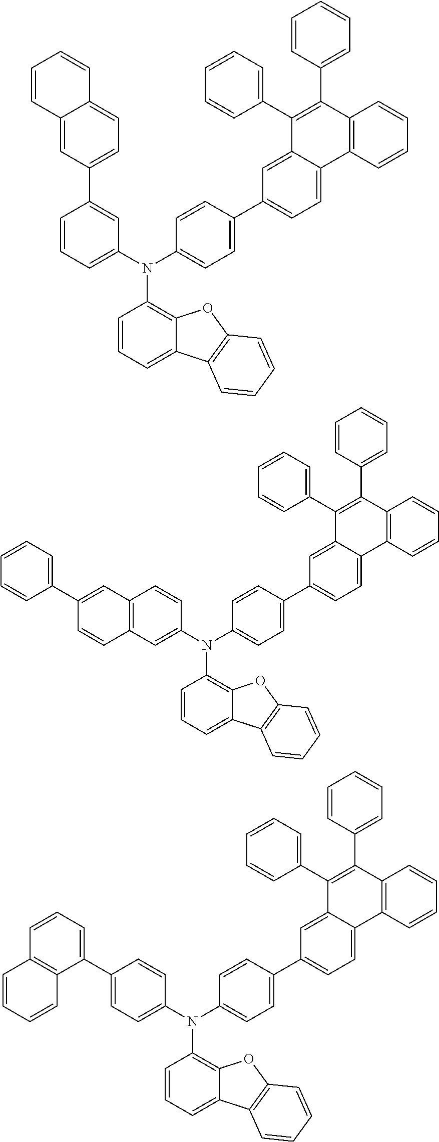

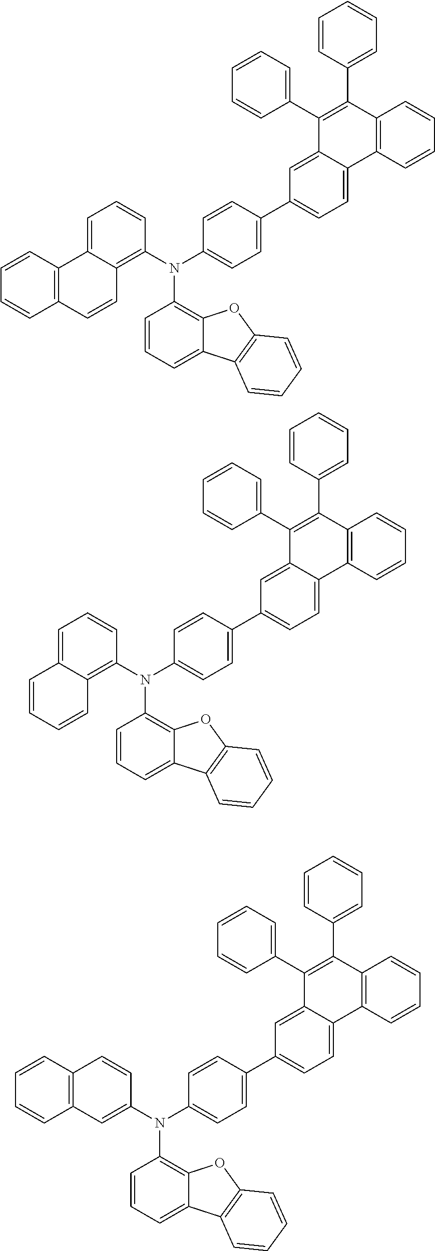

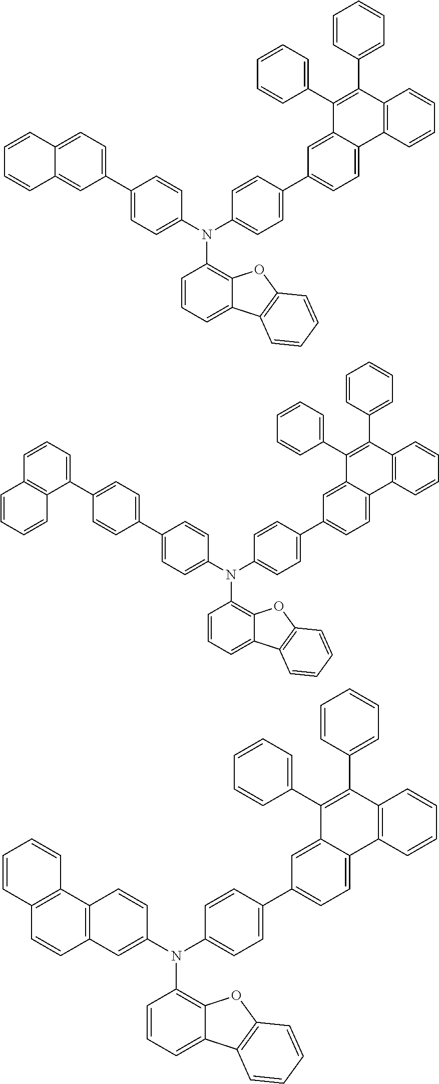

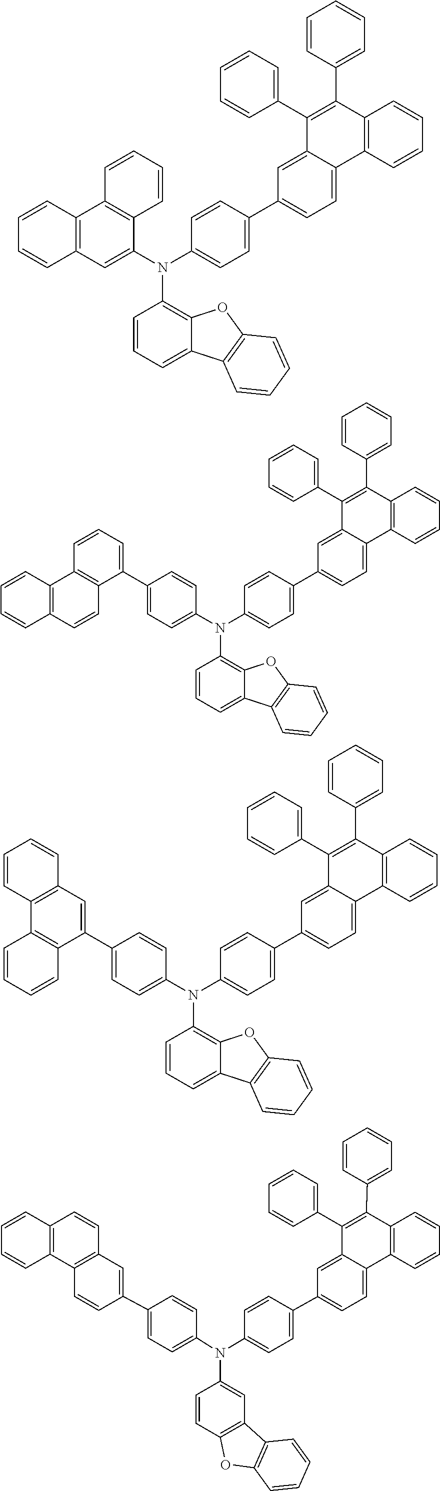

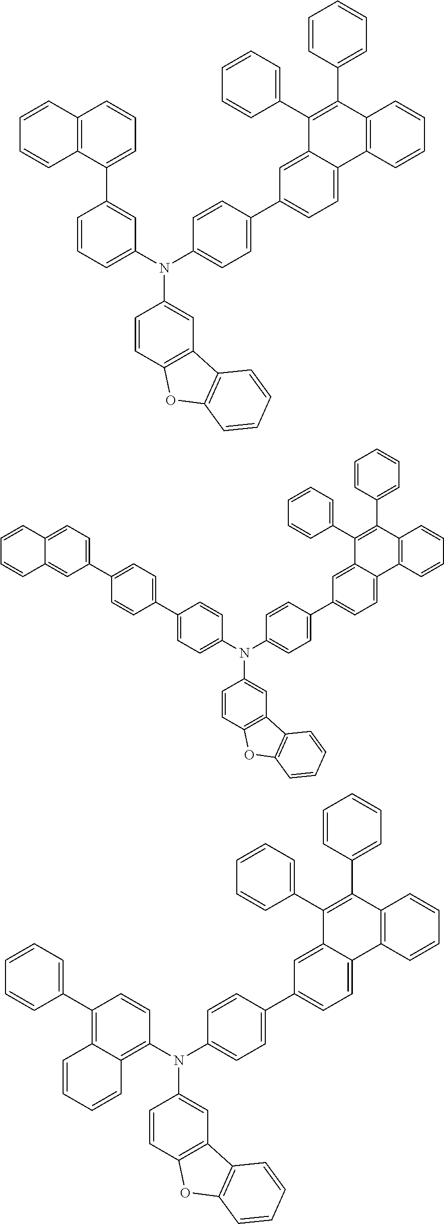

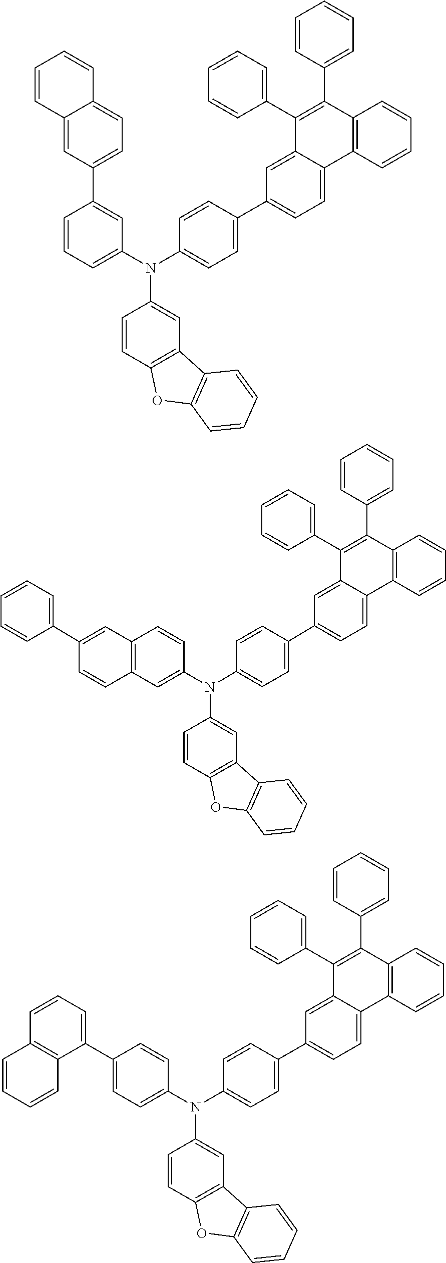 [SMILES: c1ccc(-c2c(-c3ccccc3)c3cc(-c4ccc(N(c5cccc(-c6ccc7ccccc7c6)c5)c5ccc6oc7ccccc7c6c5)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4ccc5oc6ccccc6c5c4)ccc3c2)cc1 ; c1ccc(-c2c(-c3ccccc3)c3cc(-c4ccc(N(c5ccc(-c6cccc7ccccc67)cc5)c5ccc6oc7ccccc7c6c5)cc4)ccc3c3ccccc23)cc1]

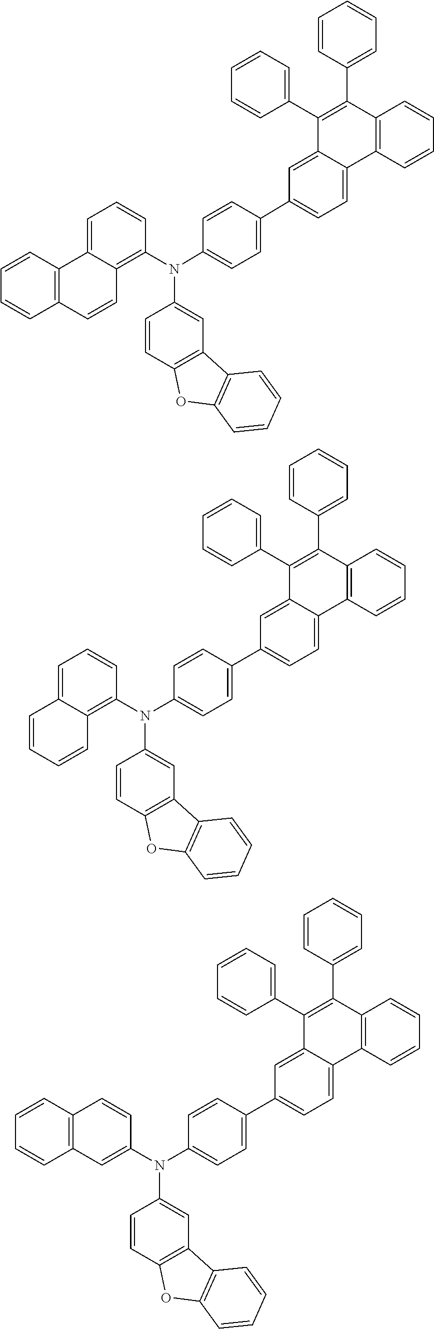

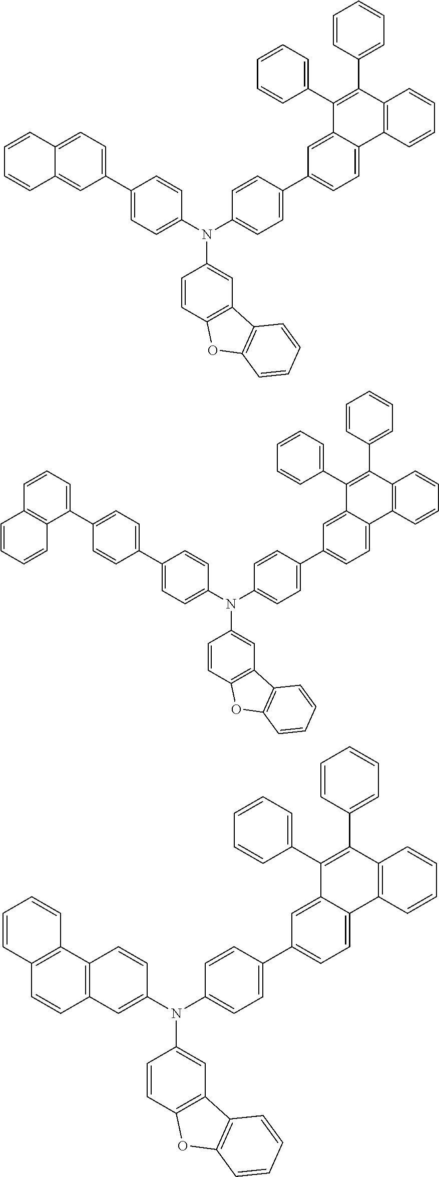

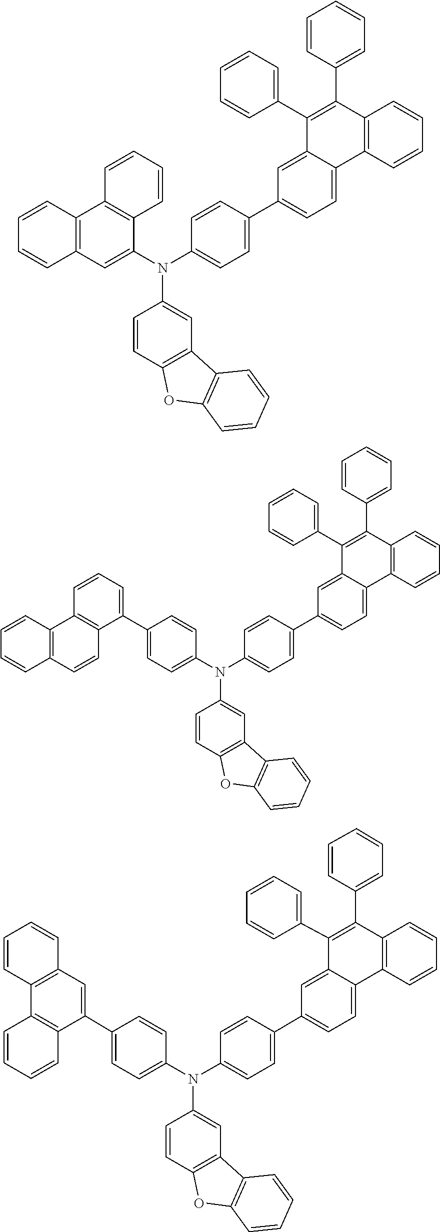 [SMILES: c1ccc(-c2c(-c3ccccc3)c3cc(-c4ccc(N(c5ccc6oc7ccccc7c6c5)c5cc6ccccc6c6ccccc56)cc4)ccc3c3ccccc23)cc1 ; c1ccc(-c2c(-c3ccccc3)c3cc(-c4ccc(N(c5ccc(-c6cccc7c6ccc6ccccc67)cc5)c5ccc6oc7ccccc7c6c5)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc6oc7ccccc7c6c5)cc4)ccc3c3ccccc23)cc1]

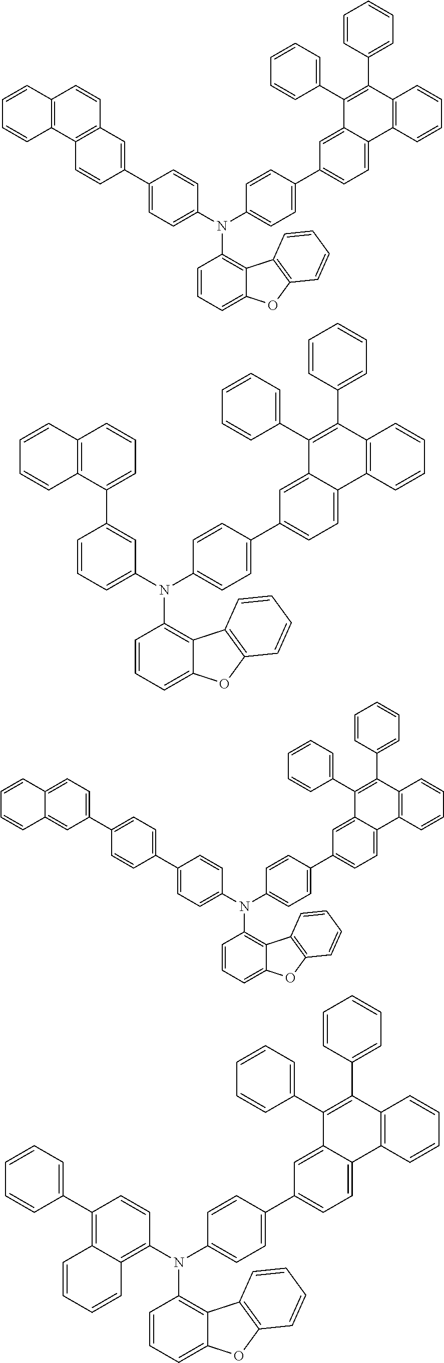 [SMILES: c1ccc(-c2c(-c3ccccc3)c3cc(-c4ccc(N(c5ccc(-c6ccc7c(ccc8ccccc87)c6)cc5)c5cccc6oc7ccccc7c56)cc4)ccc3c3ccccc23)cc1 ; c1ccc(-c2c(-c3ccccc3)c3cc(-c4ccc(N(c5cccc(-c6cccc7ccccc67)c5)c5cccc6oc7ccccc7c56)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5cccc6oc7ccccc7c56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)c3ccccc23)cc1]

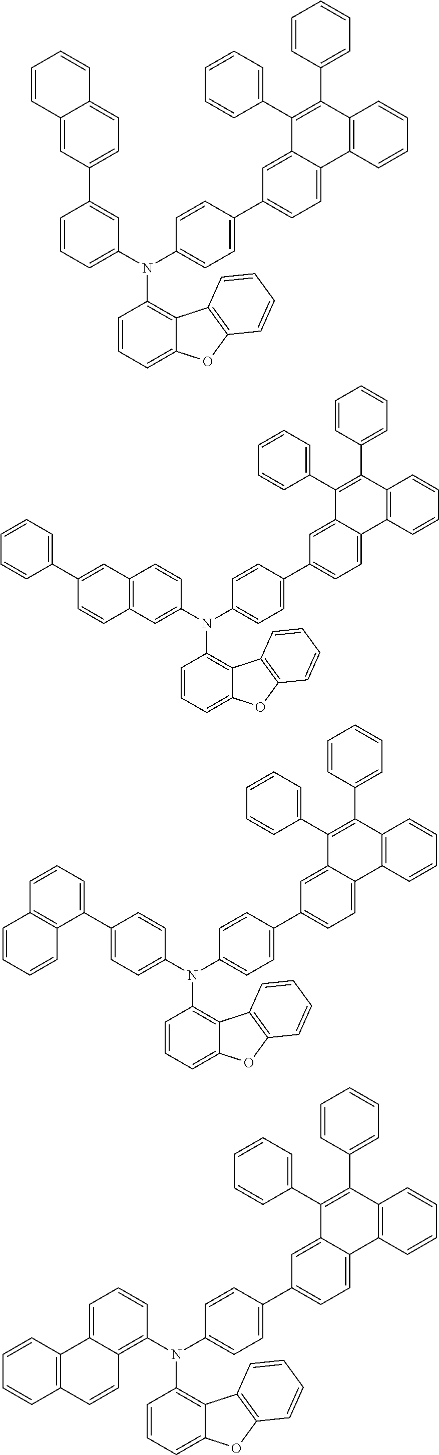 [SMILES: c1ccc(-c2c(-c3ccccc3)c3cc(-c4ccc(N(c5cccc(-c6ccc7ccccc7c6)c5)c5cccc6oc7ccccc7c56)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4cccc5oc6ccccc6c45)ccc3c2)cc1 ; c1ccc(-c2c(-c3ccccc3)c3cc(-c4ccc(N(c5ccc(-c6cccc7ccccc67)cc5)c5cccc6oc7ccccc7c56)cc4)ccc3c3ccccc23)cc1 ; c1ccc(-c2c(-c3ccccc3)c3cc(-c4ccc(N(c5cccc6c5ccc5ccccc56)c5cccc6oc7ccccc7c56)cc4)ccc3c3ccccc23)cc1]

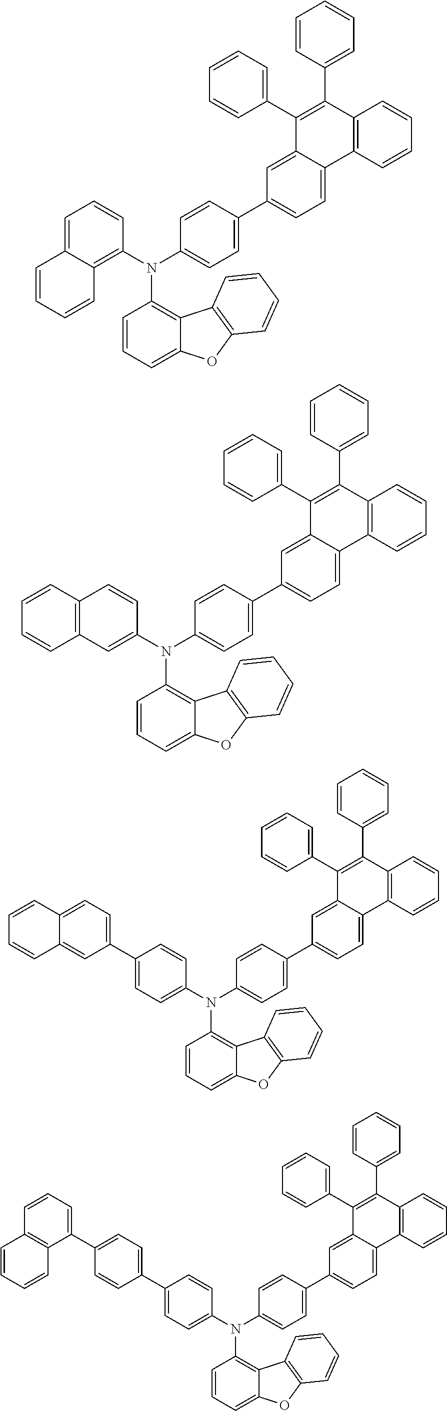 [SMILES: c1ccc(-c2c(-c3ccccc3)c3cc(-c4ccc(N(c5cccc6ccccc56)c5cccc6oc7ccccc7c56)cc4)ccc3c3ccccc23)cc1 ; c1ccc(-c2c(-c3ccccc3)c3cc(-c4ccc(N(c5ccc6ccccc6c5)c5cccc6oc7ccccc7c56)cc4)ccc3c3ccccc23)cc1 ; c1ccc(-c2c(-c3ccccc3)c3cc(-c4ccc(N(c5ccc(-c6ccc7ccccc7c6)cc5)c5cccc6oc7ccccc7c56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cccc6oc7ccccc7c56)cc4)ccc3c3ccccc23)cc1]

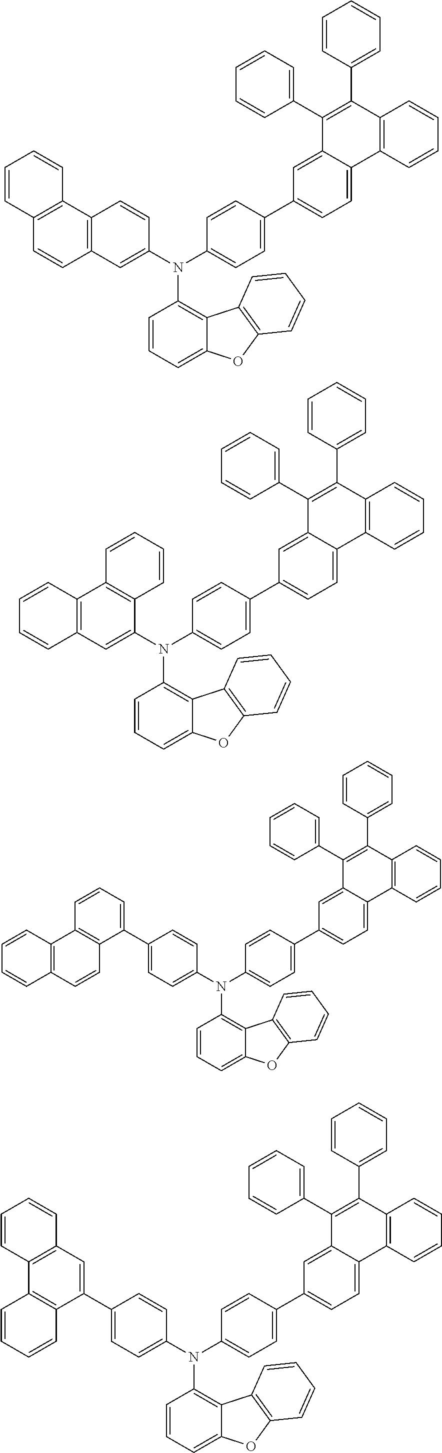 [SMILES: c1ccc(-c2c(-c3ccccc3)c3cc(-c4ccc(N(c5ccc6c(ccc7ccccc76)c5)c5cccc6oc7ccccc7c56)cc4)ccc3c3ccccc23)cc1 ; c1ccc(-c2c(-c3ccccc3)c3cc(-c4ccc(N(c5cc6ccccc6c6ccccc56)c5cccc6oc7ccccc7c56)cc4)ccc3c3ccccc23)cc1 ; c1ccc(-c2c(-c3ccccc3)c3cc(-c4ccc(N(c5ccc(-c6cccc7c6ccc6ccccc67)cc5)c5cccc6oc7ccccc7c56)cc4)ccc3c3ccccc23)cc1 ; c1ccc(-c2c(-c3ccccc3)c3cc(-c4ccc(N(c5ccc(-c6cc7ccccc7c7ccccc67)cc5)c5cccc6oc7ccccc7c56)cc4)ccc3c3ccccc23)cc1]

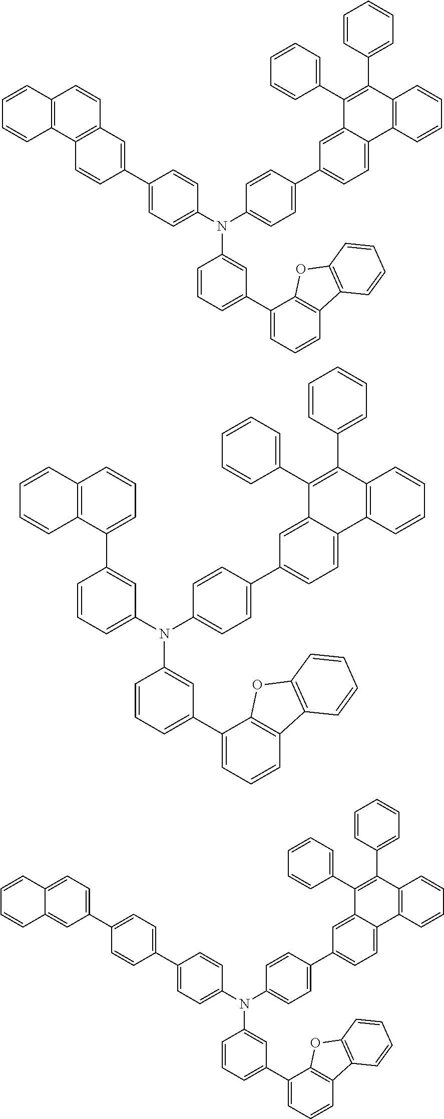

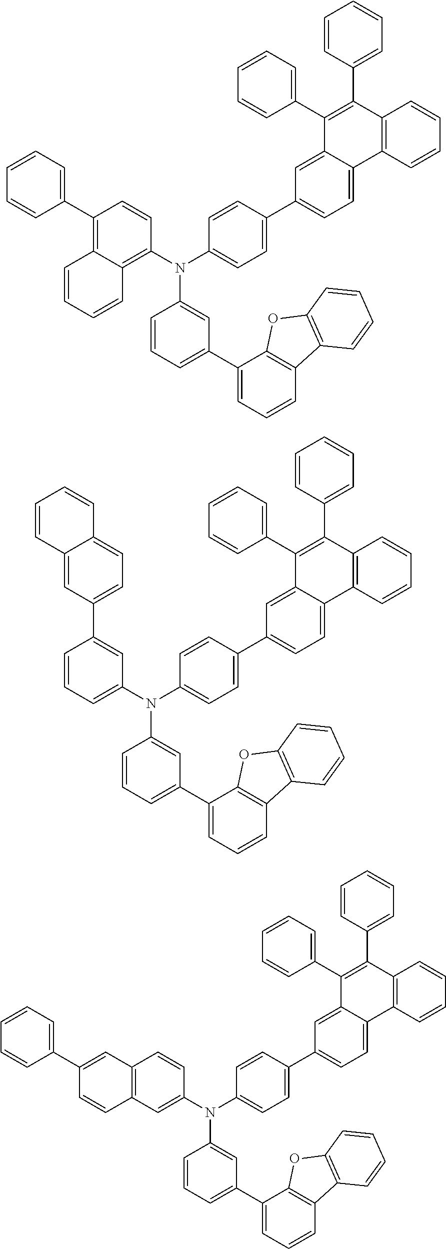

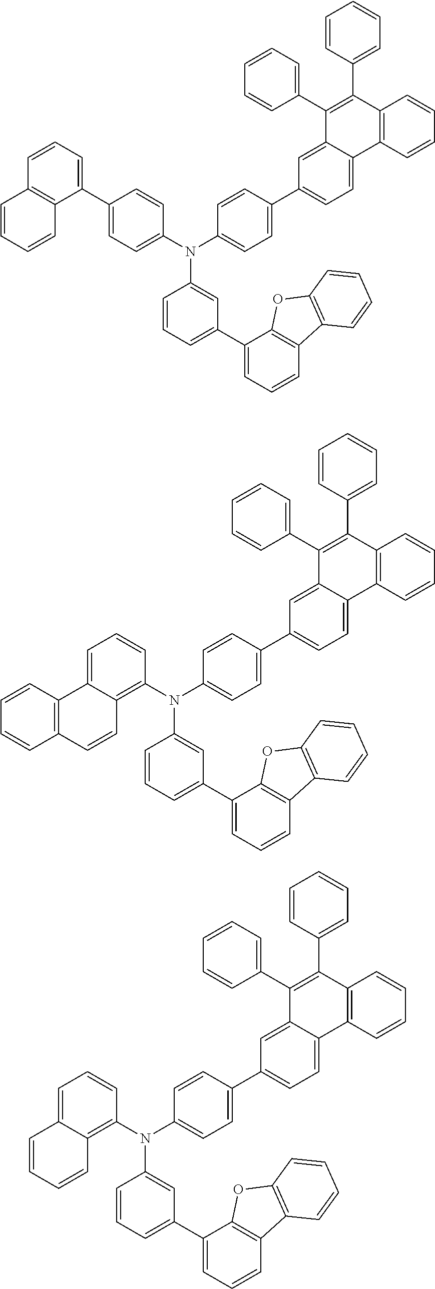 [SMILES: c1ccc(-c2c(-c3ccccc3)c3cc(-c4ccc(N(c5ccc(-c6cccc7ccccc67)cc5)c5cccc(-c6cccc7c6oc6ccccc67)c5)cc4)ccc3c3ccccc23)cc1 ; c1ccc(-c2c(-c3ccccc3)c3cc(-c4ccc(N(c5cccc(-c6cccc7c6oc6ccccc67)c5)c5cccc6c5ccc5ccccc56)cc4)ccc3c3ccccc23)cc1 ; c1ccc(-c2c(-c3ccccc3)c3cc(-c4ccc(N(c5cccc(-c6cccc7c6oc6ccccc67)c5)c5cccc6ccccc56)cc4)ccc3c3ccccc23)cc1]

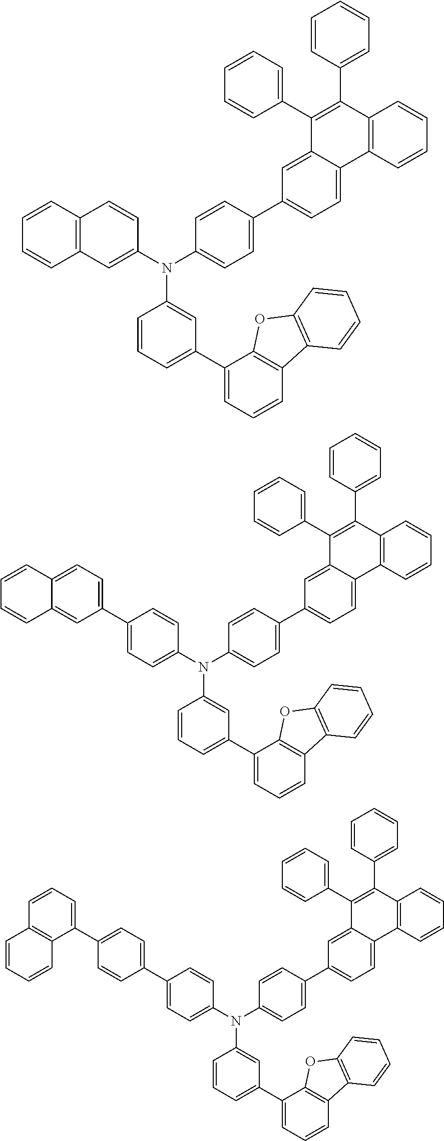 [SMILES: c1ccc(-c2c(-c3ccccc3)c3cc(-c4ccc(N(c5cccc(-c6cccc7c6oc6ccccc67)c5)c5ccc6ccccc6c5)cc4)ccc3c3ccccc23)cc1 ; c1ccc(-c2c(-c3ccccc3)c3cc(-c4ccc(N(c5ccc(-c6ccc7ccccc7c6)cc5)c5cccc(-c6cccc7c6oc6ccccc67)c5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cccc(-c6cccc7c6oc6ccccc67)c5)cc4)ccc3c3ccccc23)cc1]

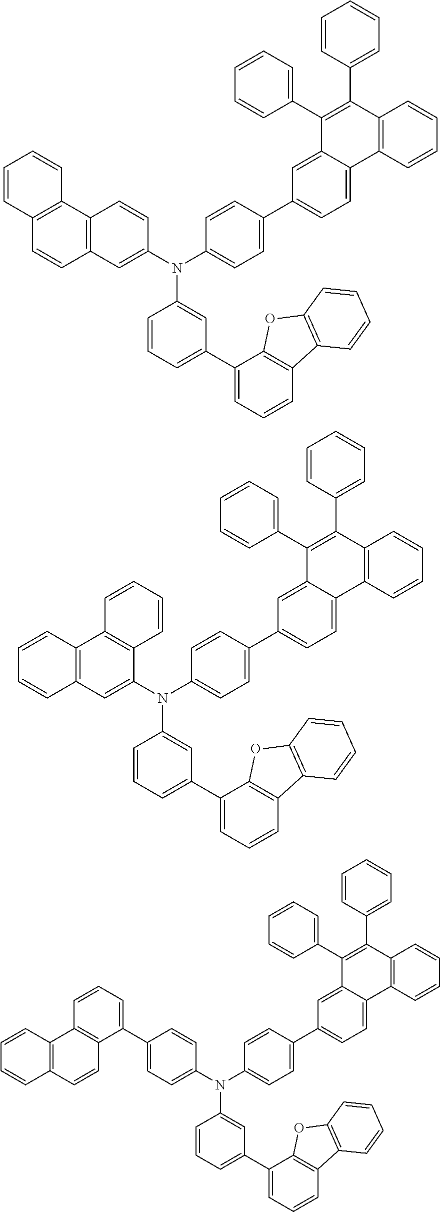 [SMILES: c1ccc(-c2c(-c3ccccc3)c3cc(-c4ccc(N(c5cccc(-c6cccc7c6oc6ccccc67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7c6oc6ccccc67)c5)c5cc6ccccc6c6ccccc56)cc4)ccc3c3ccccc23)cc1 ; c1ccc(-c2c(-c3ccccc3)c3cc(-c4ccc(N(c5ccc(-c6cccc7c6ccc6ccccc67)cc5)c5cccc(-c6cccc7c6oc6ccccc67)c5)cc4)ccc3c3ccccc23)cc1]

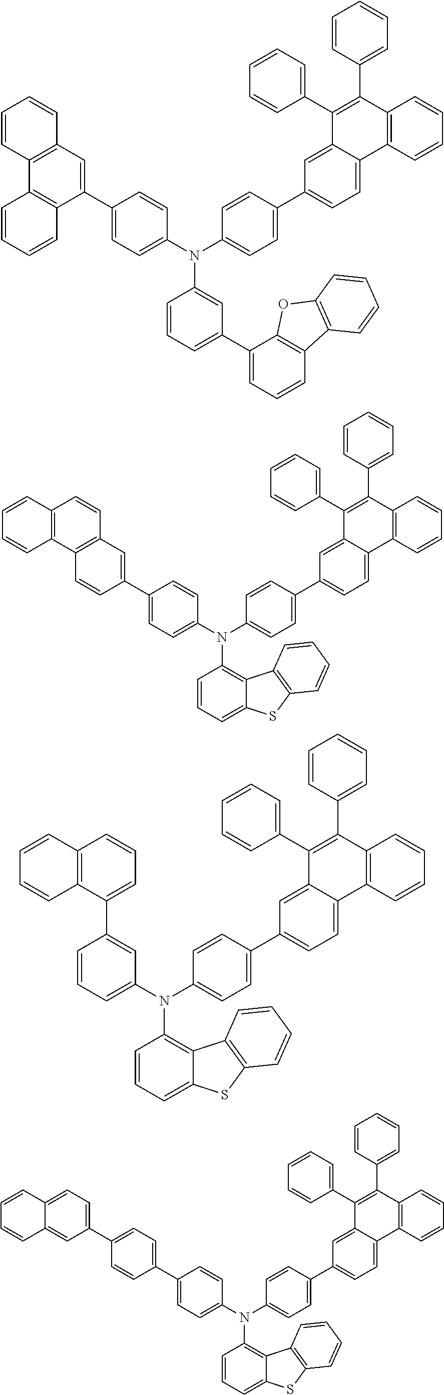 [SMILES: c1ccc(-c2c(-c3ccccc3)c3cc(-c4ccc(N(c5ccc(-c6cc7ccccc7c7ccccc67)cc5)c5cccc(-c6cccc7c6oc6ccccc67)c5)cc4)ccc3c3ccccc23)cc1 ; c1ccc(-c2c(-c3ccccc3)c3cc(-c4ccc(N(c5ccc(-c6ccc7c(ccc8ccccc87)c6)cc5)c5cccc6sc7ccccc7c56)cc4)ccc3c3ccccc23)cc1 ; c1ccc(-c2c(-c3ccccc3)c3cc(-c4ccc(N(c5cccc(-c6cccc7ccccc67)c5)c5cccc6sc7ccccc7c56)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5cccc6sc7ccccc7c56)cc4)ccc3c3ccccc23)cc1]

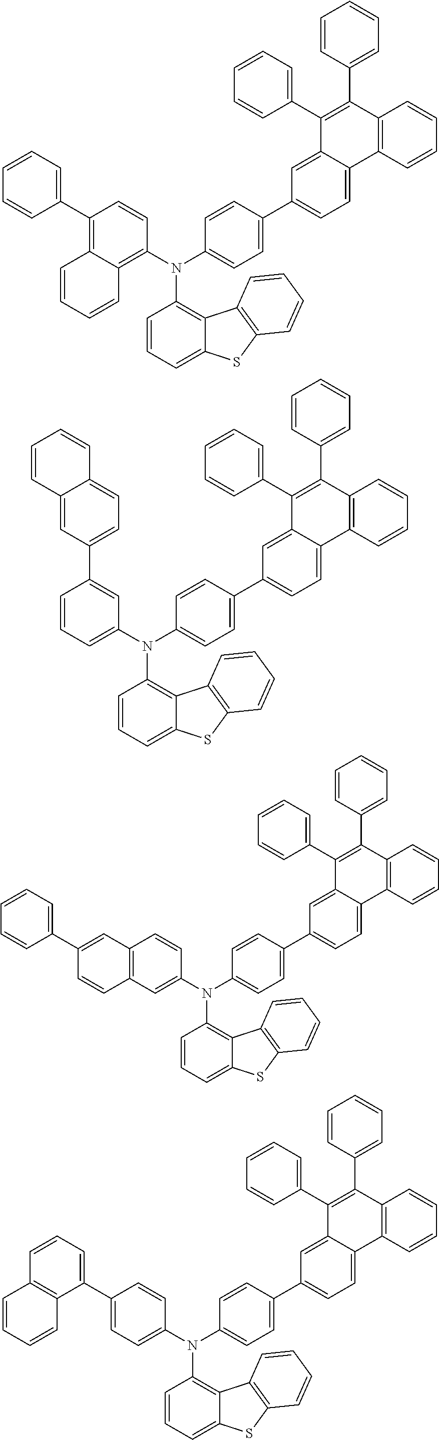 [SMILES: c1ccc(-c2ccc(N(c3ccc(-c4ccc5c(c4)c(-c4ccccc4)c(-c4ccccc4)c4ccccc45)cc3)c3cccc4sc5ccccc5c34)c3ccccc23)cc1 ; c1ccc(-c2c(-c3ccccc3)c3cc(-c4ccc(N(c5cccc(-c6ccc7ccccc7c6)c5)c5cccc6sc7ccccc7c56)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4cccc5sc6ccccc6c45)ccc3c2)cc1 ; c1ccc(-c2c(-c3ccccc3)c3cc(-c4ccc(N(c5ccc(-c6cccc7ccccc67)cc5)c5cccc6sc7ccccc7c56)cc4)ccc3c3ccccc23)cc1]

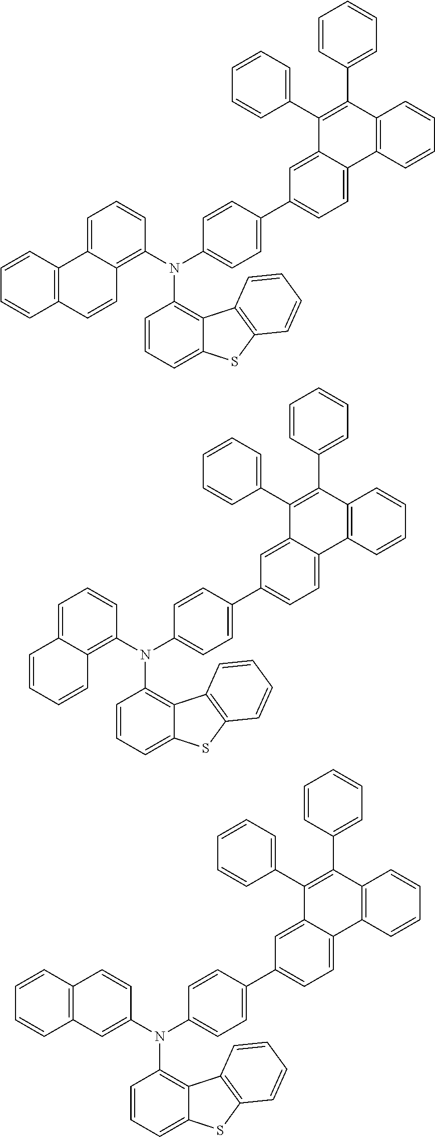 [SMILES: c1ccc(-c2c(-c3ccccc3)c3cc(-c4ccc(N(c5cccc6c5ccc5ccccc56)c5cccc6sc7ccccc7c56)cc4)ccc3c3ccccc23)cc1 ; c1ccc(-c2c(-c3ccccc3)c3cc(-c4ccc(N(c5cccc6ccccc56)c5cccc6sc7ccccc7c56)cc4)ccc3c3ccccc23)cc1 ; c1ccc(-c2c(-c3ccccc3)c3cc(-c4ccc(N(c5ccc6ccccc6c5)c5cccc6sc7ccccc7c56)cc4)ccc3c3ccccc23)cc1]

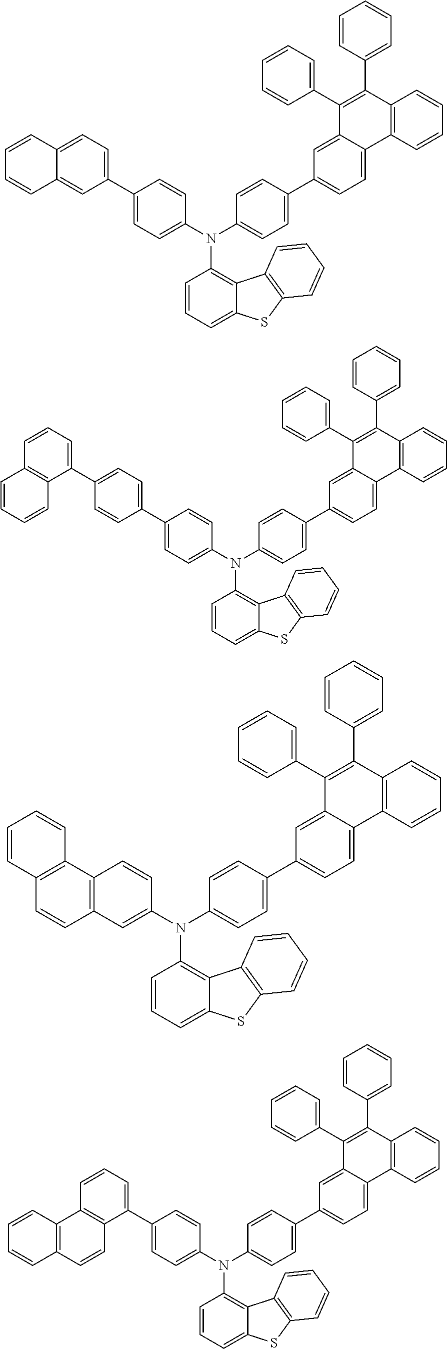 [SMILES: c1ccc(-c2c(-c3ccccc3)c3cc(-c4ccc(N(c5ccc(-c6ccc7ccccc7c6)cc5)c5cccc6sc7ccccc7c56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cccc6sc7ccccc7c56)cc4)ccc3c3ccccc23)cc1 ; c1ccc(-c2c(-c3ccccc3)c3cc(-c4ccc(N(c5ccc6c(ccc7ccccc76)c5)c5cccc6sc7ccccc7c56)cc4)ccc3c3ccccc23)cc1 ; c1ccc(-c2c(-c3ccccc3)c3cc(-c4ccc(N(c5ccc(-c6cccc7c6ccc6ccccc67)cc5)c5cccc6sc7ccccc7c56)cc4)ccc3c3ccccc23)cc1]

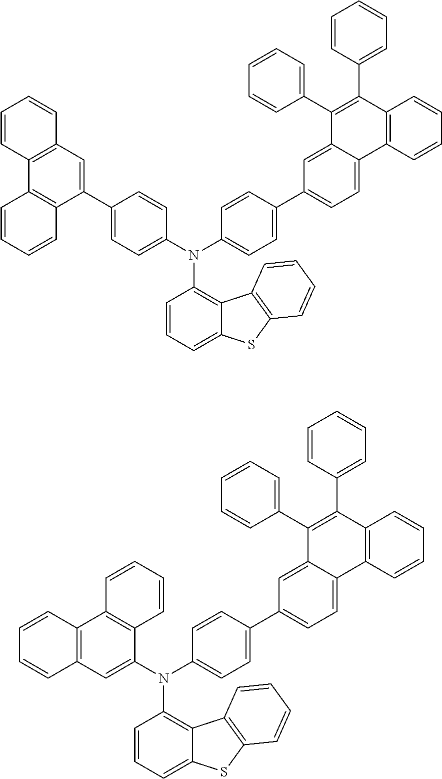 [SMILES: c1ccc(-c2c(-c3ccccc3)c3cc(-c4ccc(N(c5ccc(-c6cc7ccccc7c7ccccc67)cc5)c5cccc6sc7ccccc7c56)cc4)ccc3c3ccccc23)cc1 ; c1ccc(-c2c(-c3ccccc3)c3cc(-c4ccc(N(c5cc6ccccc6c6ccccc56)c5cccc6sc7ccccc7c56)cc4)ccc3c3ccccc23)cc1]

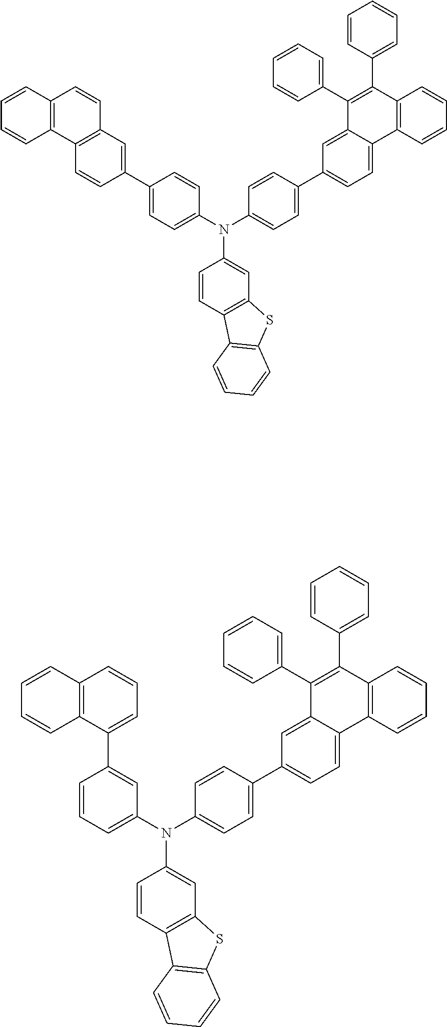 [SMILES: c1ccc(-c2c(-c3ccccc3)c3cc(-c4ccc(N(c5ccc(-c6ccc7c(ccc8ccccc87)c6)cc5)c5ccc6c(c5)sc5ccccc56)cc4)ccc3c3ccccc23)cc1 ; c1ccc(-c2c(-c3ccccc3)c3cc(-c4ccc(N(c5cccc(-c6cccc7ccccc67)c5)c5ccc6c(c5)sc5ccccc56)cc4)ccc3c3ccccc23)cc1]

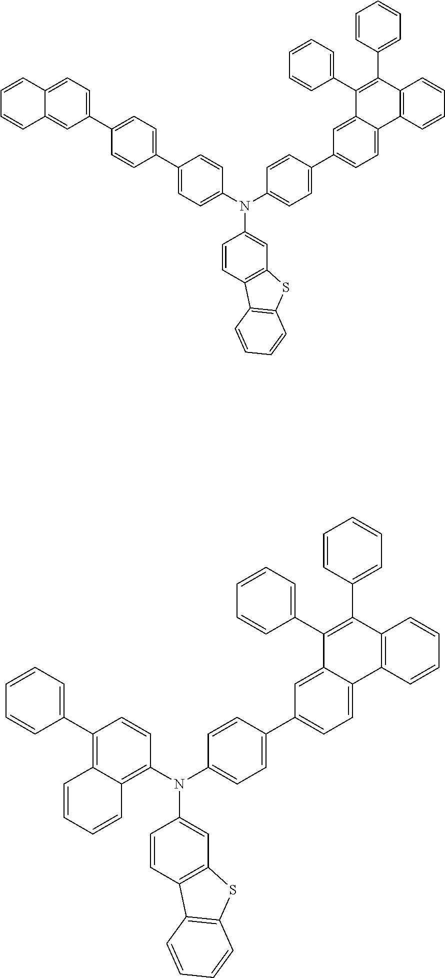 [SMILES: c1ccc(-c2c(-c3ccccc3)c3cc(-c4ccc(N(c5ccc(-c6ccc(-c7ccc8ccccc8c7)cc6)cc5)c5ccc6c(c5)sc5ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c(c3)sc3ccccc34)c3ccccc23)cc1]

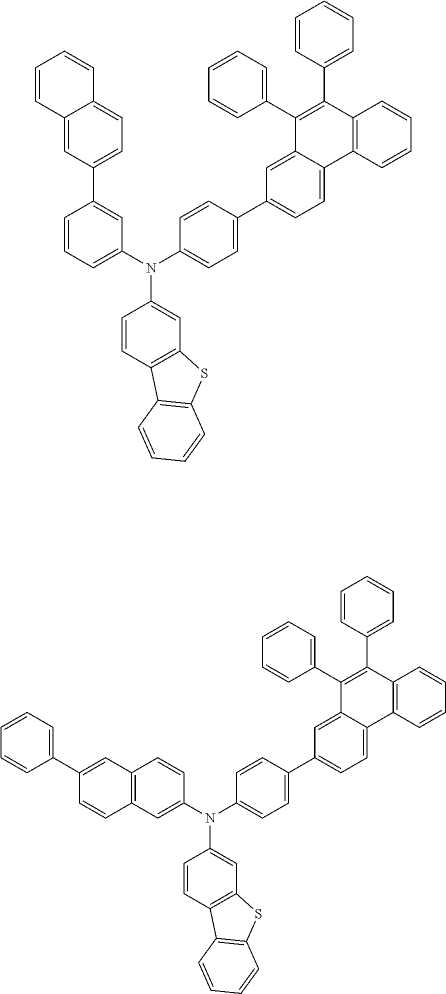 [SMILES: c1ccc(-c2c(-c3ccccc3)c3cc(-c4ccc(N(c5cccc(-c6ccc7ccccc7c6)c5)c5ccc6c(c5)sc5ccccc56)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4ccc5c(c4)sc4ccccc45)ccc3c2)cc1]

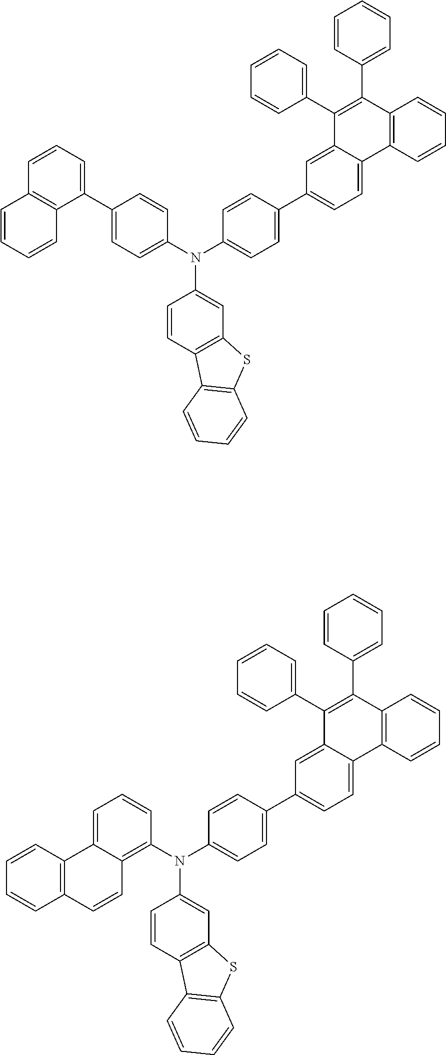 [SMILES: c1ccc(-c2c(-c3ccccc3)c3cc(-c4ccc(N(c5ccc(-c6cccc7ccccc67)cc5)c5ccc6c(c5)sc5ccccc56)cc4)ccc3c3ccccc23)cc1 ; c1ccc(-c2c(-c3ccccc3)c3cc(-c4ccc(N(c5ccc6c(c5)sc5ccccc56)c5cccc6c5ccc5ccccc56)cc4)ccc3c3ccccc23)cc1]

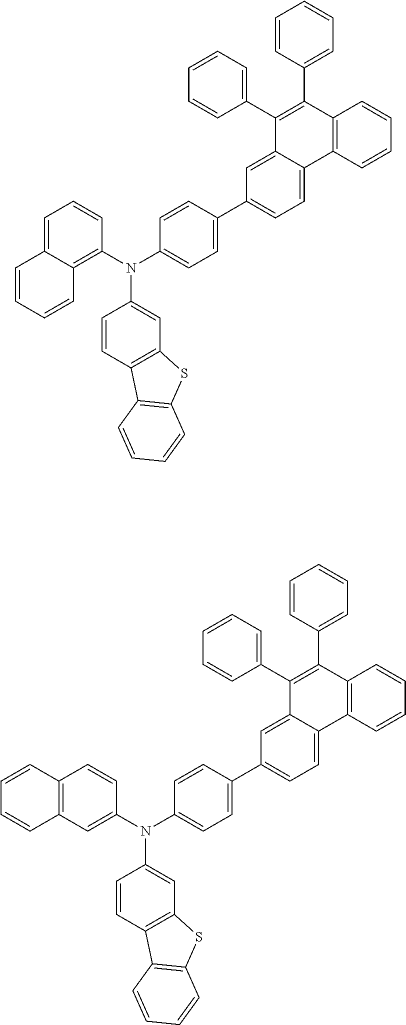 [SMILES: c1ccc(-c2c(-c3ccccc3)c3cc(-c4ccc(N(c5ccc6c(c5)sc5ccccc56)c5cccc6ccccc56)cc4)ccc3c3ccccc23)cc1 ; c1ccc(-c2c(-c3ccccc3)c3cc(-c4ccc(N(c5ccc6ccccc6c5)c5ccc6c(c5)sc5ccccc56)cc4)ccc3c3ccccc23)cc1]

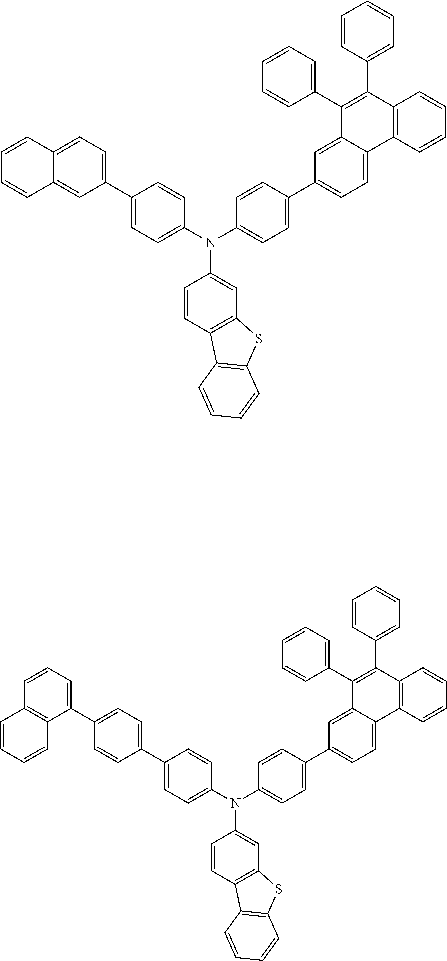 [SMILES: c1ccc(-c2c(-c3ccccc3)c3cc(-c4ccc(N(c5ccc(-c6ccc7ccccc7c6)cc5)c5ccc6c(c5)sc5ccccc56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc6c(c5)sc5ccccc56)cc4)ccc3c3ccccc23)cc1]

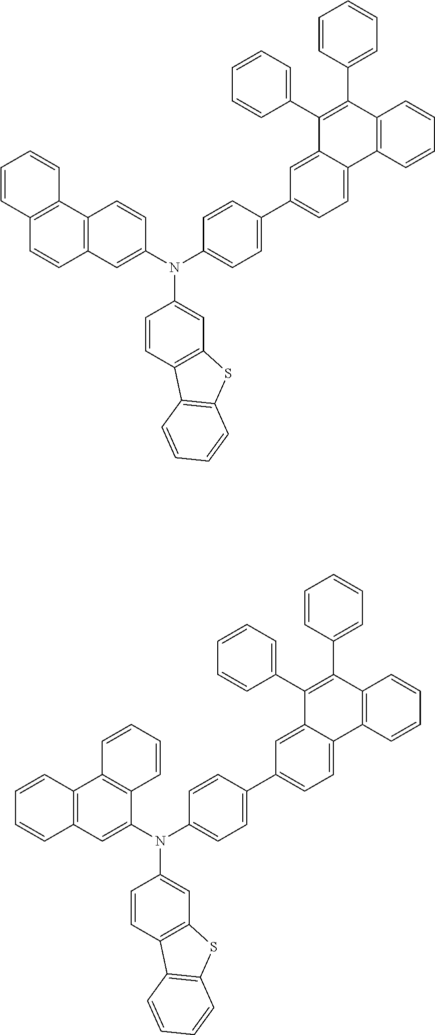 [SMILES: c1ccc(-c2c(-c3ccccc3)c3cc(-c4ccc(N(c5ccc6c(ccc7ccccc76)c5)c5ccc6c(c5)sc5ccccc56)cc4)ccc3c3ccccc23)cc1 ; c1ccc(-c2c(-c3ccccc3)c3cc(-c4ccc(N(c5ccc6c(c5)sc5ccccc56)c5cc6ccccc6c6ccccc56)cc4)ccc3c3ccccc23)cc1]

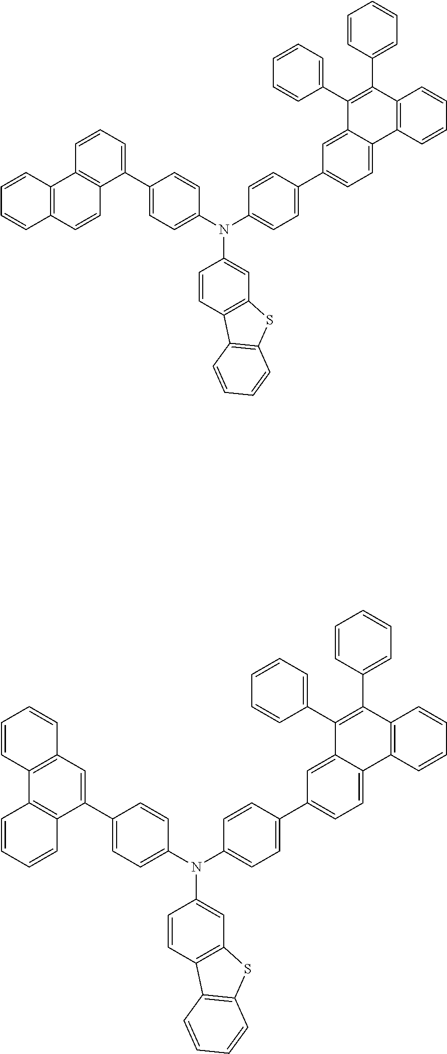 [SMILES: c1ccc(-c2c(-c3ccccc3)c3cc(-c4ccc(N(c5ccc(-c6cccc7c6ccc6ccccc67)cc5)c5ccc6c(c5)sc5ccccc56)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc6c(c5)sc5ccccc56)cc4)ccc3c3ccccc23)cc1]

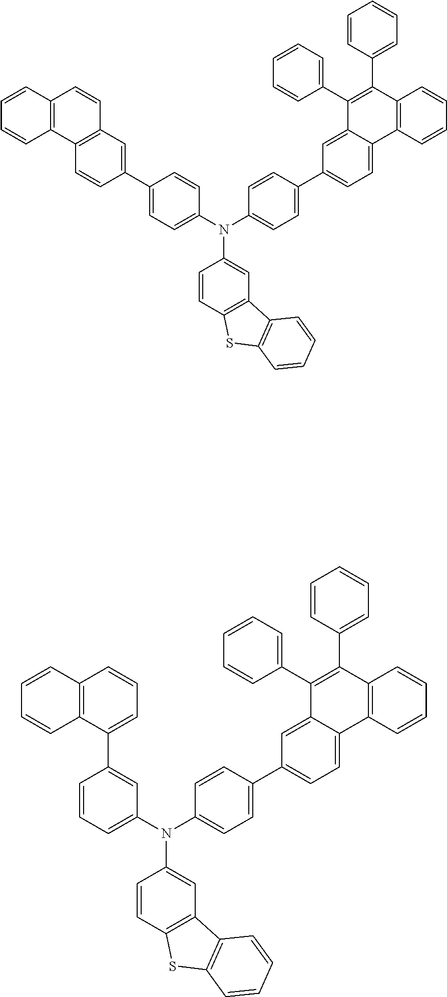 [SMILES: c1ccc(-c2c(-c3ccccc3)c3cc(-c4ccc(N(c5ccc(-c6ccc7c(ccc8ccccc87)c6)cc5)c5ccc6sc7ccccc7c6c5)cc4)ccc3c3ccccc23)cc1 ; c1ccc(-c2c(-c3ccccc3)c3cc(-c4ccc(N(c5cccc(-c6cccc7ccccc67)c5)c5ccc6sc7ccccc7c6c5)cc4)ccc3c3ccccc23)cc1]

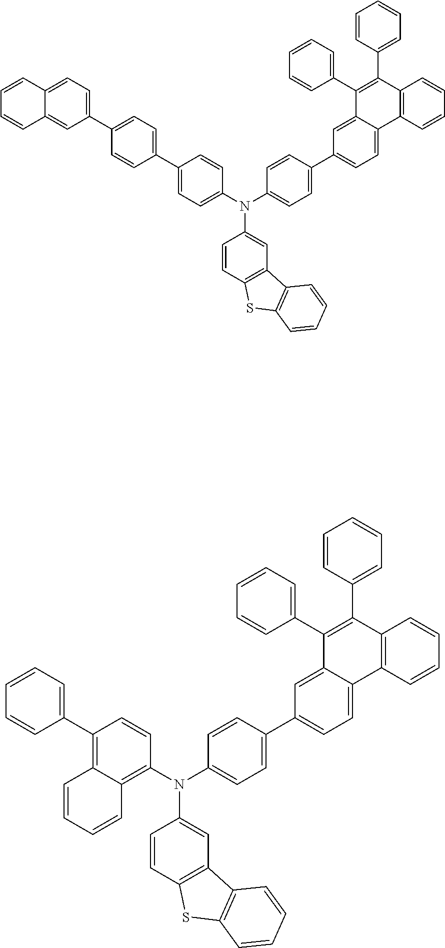 [SMILES: c1ccc(-c2c(-c3ccccc3)c3cc(-c4ccc(N(c5ccc(-c6ccc(-c7ccc8ccccc8c7)cc6)cc5)c5ccc6sc7ccccc7c6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4sc5ccccc5c4c3)c3ccccc23)cc1]

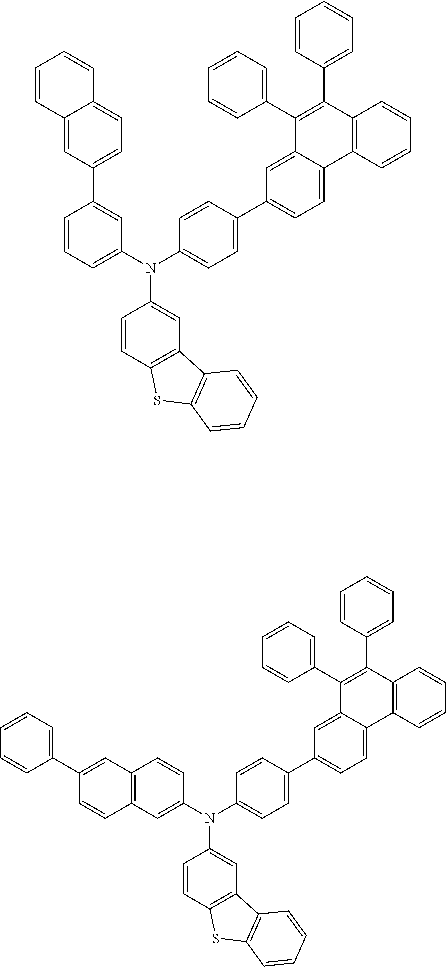 [SMILES: c1ccc(-c2c(-c3ccccc3)c3cc(-c4ccc(N(c5cccc(-c6ccc7ccccc7c6)c5)c5ccc6sc7ccccc7c6c5)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4ccc5sc6ccccc6c5c4)ccc3c2)cc1]

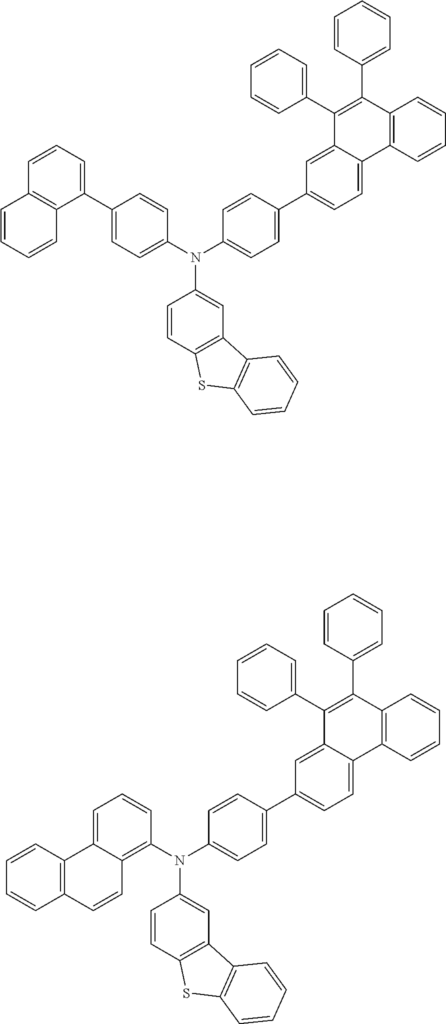 [SMILES: c1ccc(-c2c(-c3ccccc3)c3cc(-c4ccc(N(c5ccc(-c6cccc7ccccc67)cc5)c5ccc6sc7ccccc7c6c5)cc4)ccc3c3ccccc23)cc1 ; c1ccc(-c2c(-c3ccccc3)c3cc(-c4ccc(N(c5ccc6sc7ccccc7c6c5)c5cccc6c5ccc5ccccc56)cc4)ccc3c3ccccc23)cc1]

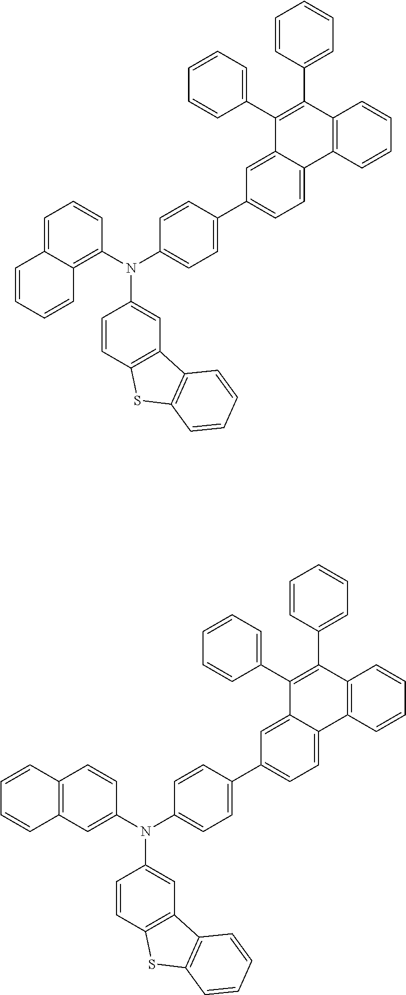 [SMILES: c1ccc(-c2c(-c3ccccc3)c3cc(-c4ccc(N(c5ccc6sc7ccccc7c6c5)c5cccc6ccccc56)cc4)ccc3c3ccccc23)cc1 ; c1ccc(-c2c(-c3ccccc3)c3cc(-c4ccc(N(c5ccc6ccccc6c5)c5ccc6sc7ccccc7c6c5)cc4)ccc3c3ccccc23)cc1]

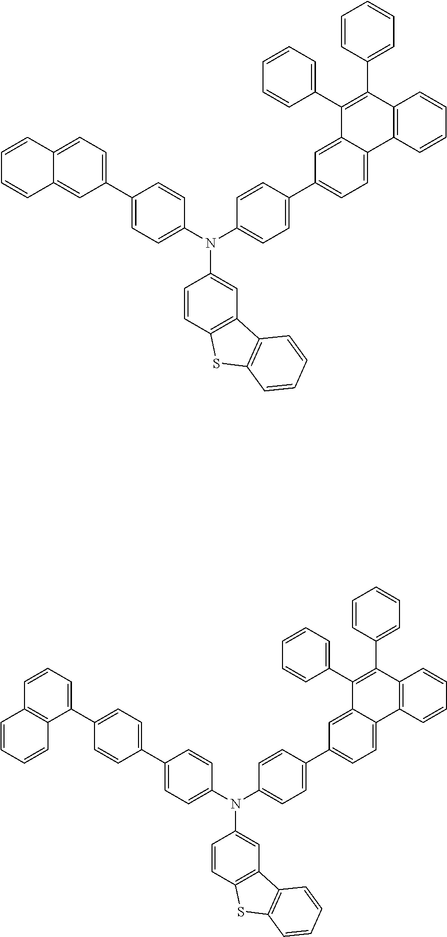 [SMILES: c1ccc(-c2c(-c3ccccc3)c3cc(-c4ccc(N(c5ccc(-c6ccc7ccccc7c6)cc5)c5ccc6sc7ccccc7c6c5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc6sc7ccccc7c6c5)cc4)ccc3c3ccccc23)cc1]

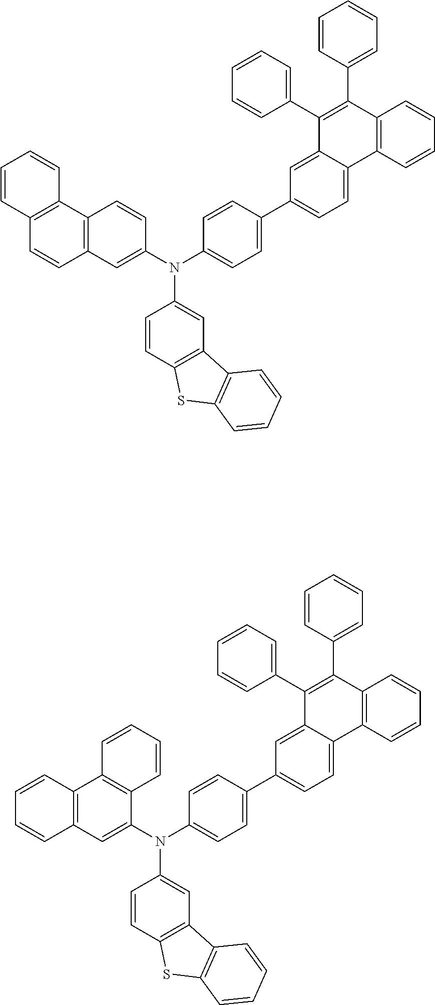 [SMILES: c1ccc(-c2c(-c3ccccc3)c3cc(-c4ccc(N(c5ccc6c(ccc7ccccc76)c5)c5ccc6sc7ccccc7c6c5)cc4)ccc3c3ccccc23)cc1 ; c1ccc(-c2c(-c3ccccc3)c3cc(-c4ccc(N(c5ccc6sc7ccccc7c6c5)c5cc6ccccc6c6ccccc56)cc4)ccc3c3ccccc23)cc1]

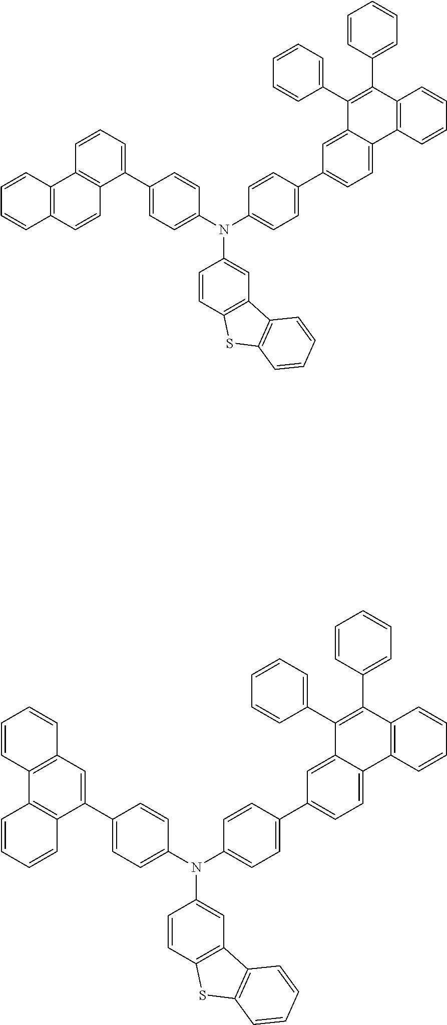 [SMILES: c1ccc(-c2c(-c3ccccc3)c3cc(-c4ccc(N(c5ccc(-c6cccc7c6ccc6ccccc67)cc5)c5ccc6sc7ccccc7c6c5)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc6sc7ccccc7c6c5)cc4)ccc3c3ccccc23)cc1]

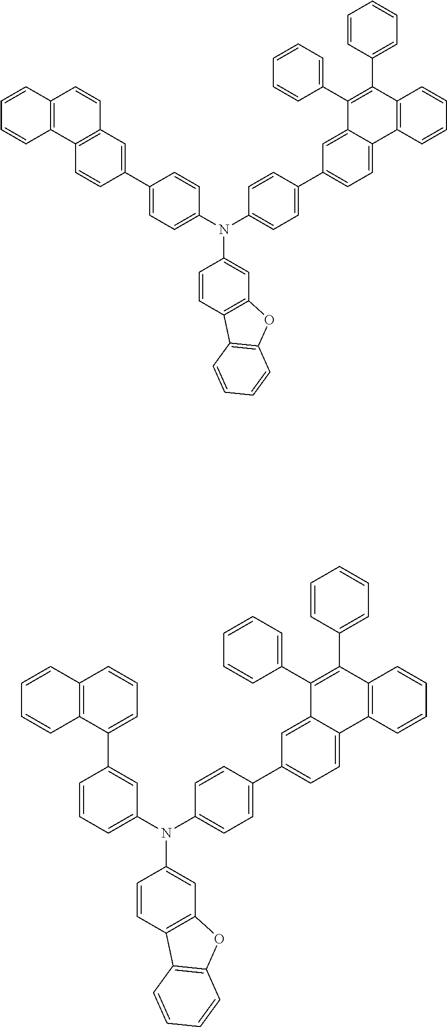 [SMILES: c1ccc(-c2c(-c3ccccc3)c3cc(-c4ccc(N(c5ccc(-c6ccc7c(ccc8ccccc87)c6)cc5)c5ccc6c(c5)oc5ccccc56)cc4)ccc3c3ccccc23)cc1 ; c1ccc(-c2c(-c3ccccc3)c3cc(-c4ccc(N(c5cccc(-c6cccc7ccccc67)c5)c5ccc6c(c5)oc5ccccc56)cc4)ccc3c3ccccc23)cc1]

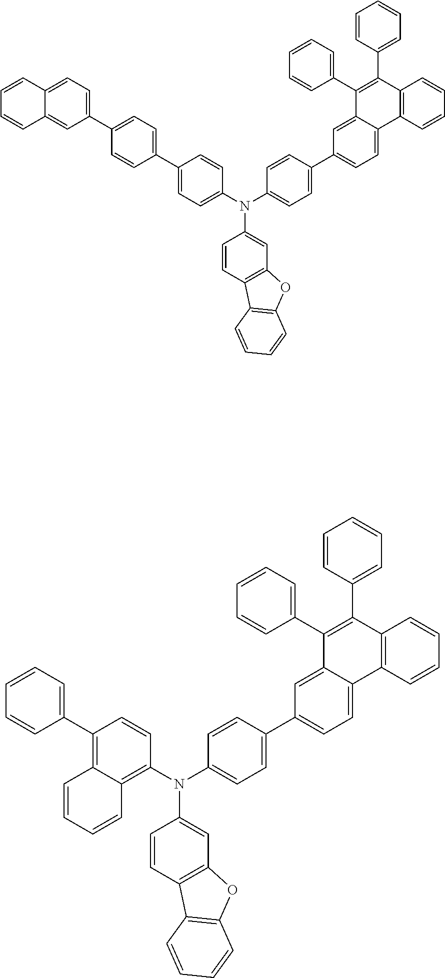 [SMILES: c1ccc(-c2c(-c3ccccc3)c3cc(-c4ccc(N(c5ccc(-c6ccc(-c7ccc8ccccc8c7)cc6)cc5)c5ccc6c(c5)oc5ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c(c3)oc3ccccc34)c3ccccc23)cc1]

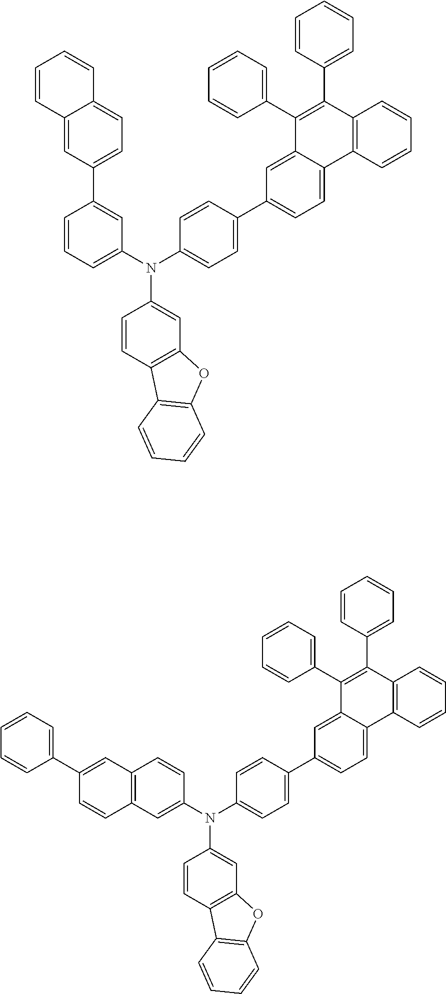 [SMILES: c1ccc(-c2c(-c3ccccc3)c3cc(-c4ccc(N(c5cccc(-c6ccc7ccccc7c6)c5)c5ccc6c(c5)oc5ccccc56)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4ccc5c(c4)oc4ccccc45)ccc3c2)cc1]

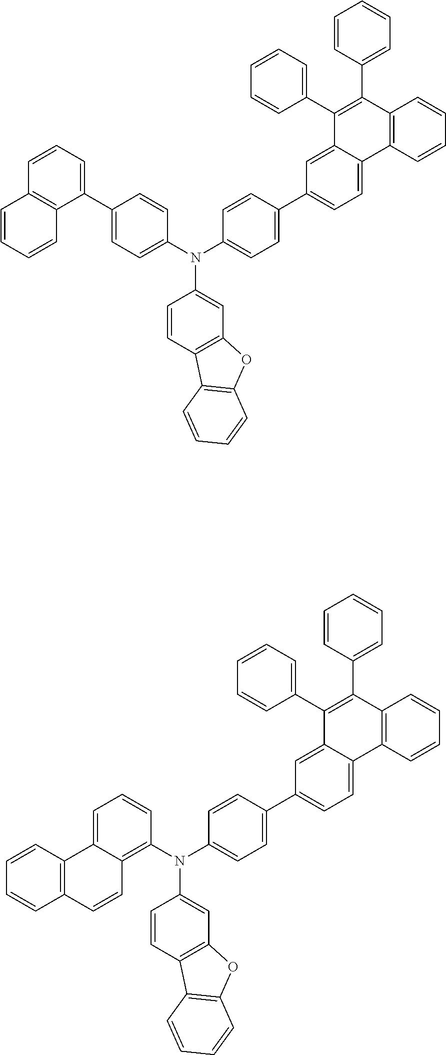 [SMILES: c1ccc(-c2c(-c3ccccc3)c3cc(-c4ccc(N(c5ccc(-c6cccc7ccccc67)cc5)c5ccc6c(c5)oc5ccccc56)cc4)ccc3c3ccccc23)cc1 ; c1ccc(-c2c(-c3ccccc3)c3cc(-c4ccc(N(c5ccc6c(c5)oc5ccccc56)c5cccc6c5ccc5ccccc56)cc4)ccc3c3ccccc23)cc1]

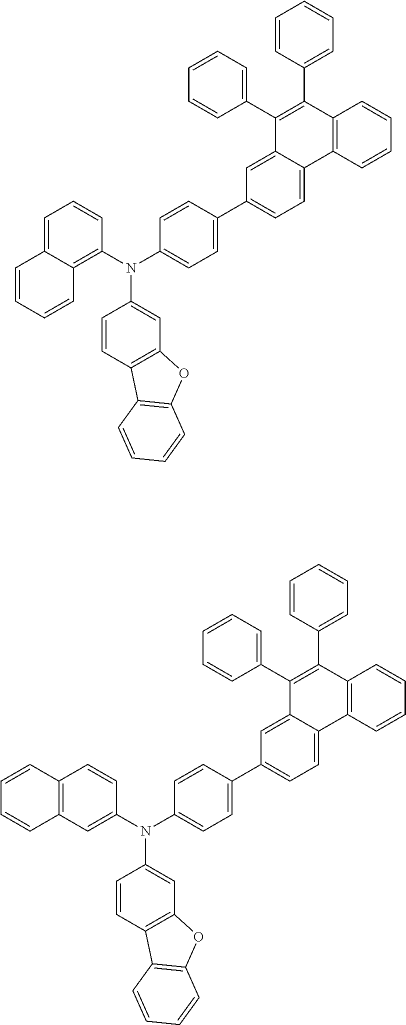 [SMILES: c1ccc(-c2c(-c3ccccc3)c3cc(-c4ccc(N(c5ccc6c(c5)oc5ccccc56)c5cccc6ccccc56)cc4)ccc3c3ccccc23)cc1 ; c1ccc(-c2c(-c3ccccc3)c3cc(-c4ccc(N(c5ccc6ccccc6c5)c5ccc6c(c5)oc5ccccc56)cc4)ccc3c3ccccc23)cc1]

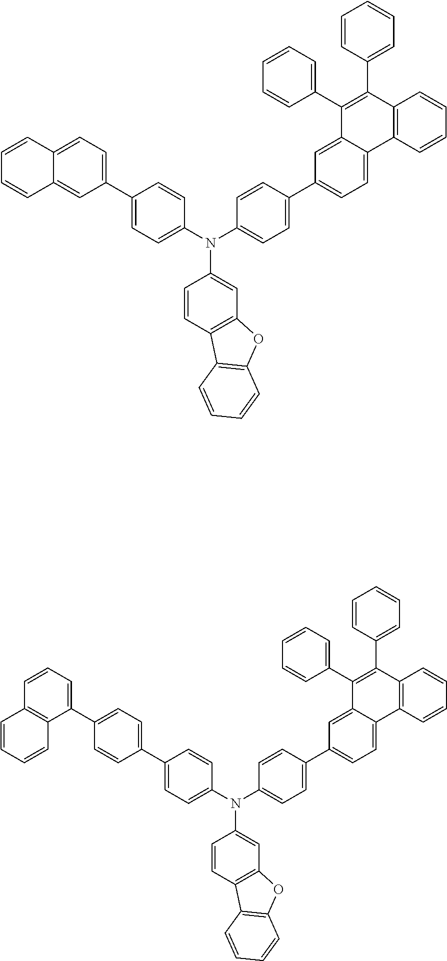 [SMILES: c1ccc(-c2c(-c3ccccc3)c3cc(-c4ccc(N(c5ccc(-c6ccc7ccccc7c6)cc5)c5ccc6c(c5)oc5ccccc56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc6c(c5)oc5ccccc56)cc4)ccc3c3ccccc23)cc1]

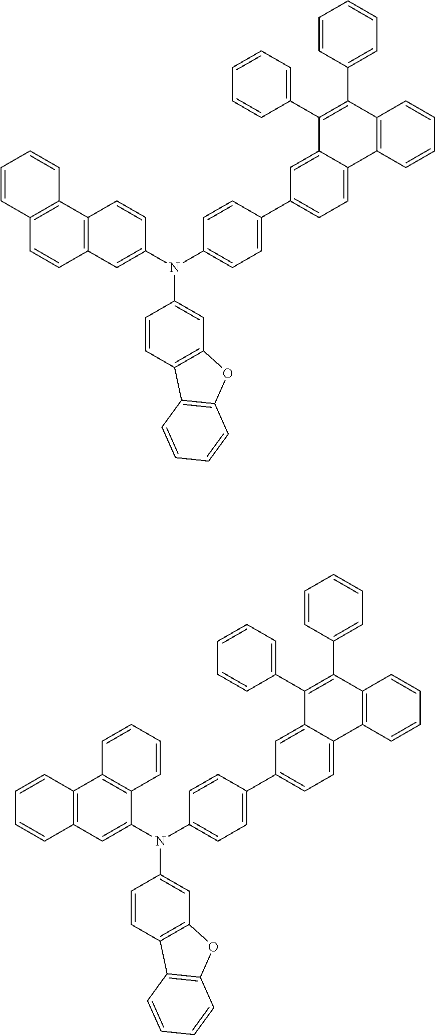 [SMILES: c1ccc(-c2c(-c3ccccc3)c3cc(-c4ccc(N(c5ccc6c(ccc7ccccc76)c5)c5ccc6c(c5)oc5ccccc56)cc4)ccc3c3ccccc23)cc1 ; c1ccc(-c2c(-c3ccccc3)c3cc(-c4ccc(N(c5ccc6c(c5)oc5ccccc56)c5cc6ccccc6c6ccccc56)cc4)ccc3c3ccccc23)cc1]

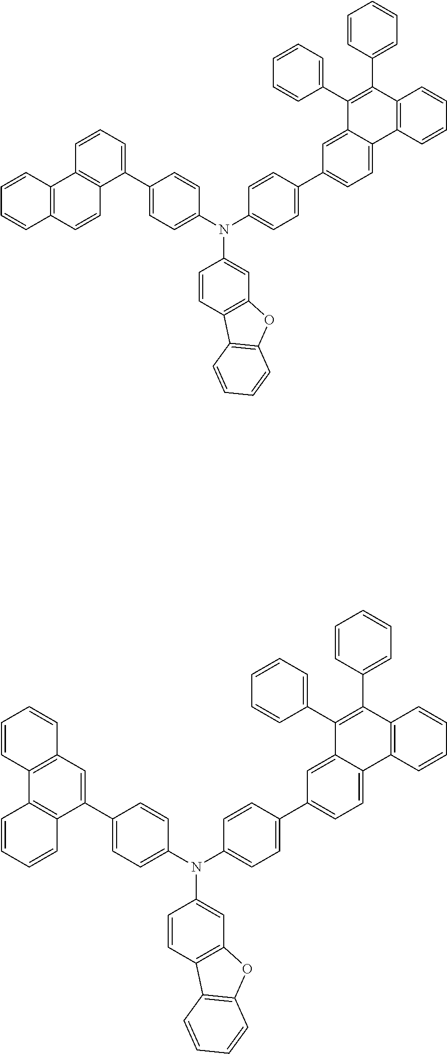 [SMILES: c1ccc(-c2c(-c3ccccc3)c3cc(-c4ccc(N(c5ccc(-c6cccc7c6ccc6ccccc67)cc5)c5ccc6c(c5)oc5ccccc56)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc6c(c5)oc5ccccc56)cc4)ccc3c3ccccc23)cc1]

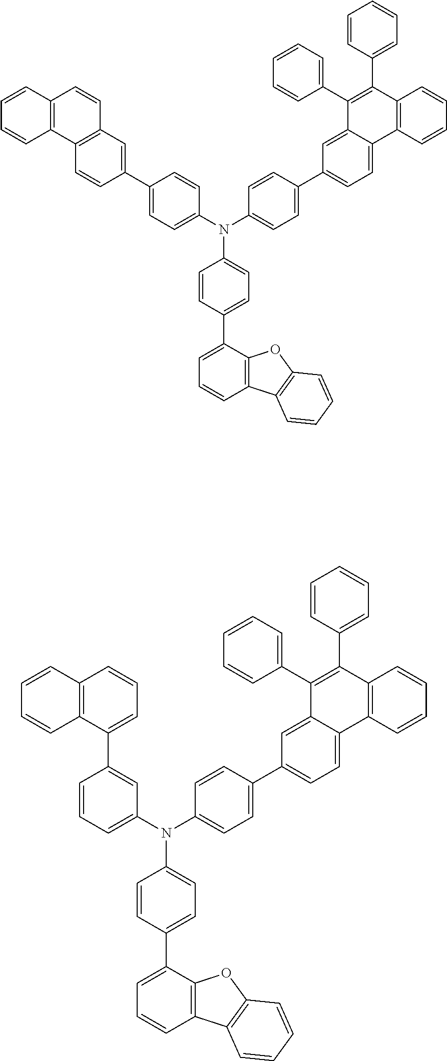 [SMILES: c1ccc(-c2c(-c3ccccc3)c3cc(-c4ccc(N(c5ccc(-c6ccc7c(ccc8ccccc87)c6)cc5)c5ccc(-c6cccc7c6oc6ccccc67)cc5)cc4)ccc3c3ccccc23)cc1 ; c1ccc(-c2c(-c3ccccc3)c3cc(-c4ccc(N(c5ccc(-c6cccc7c6oc6ccccc67)cc5)c5cccc(-c6cccc7ccccc67)c5)cc4)ccc3c3ccccc23)cc1]

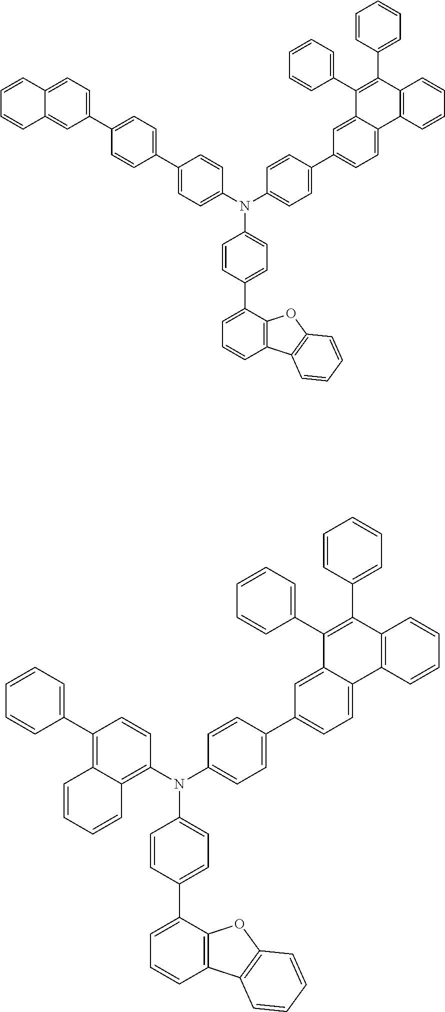 [SMILES: c1ccc(-c2c(-c3ccccc3)c3cc(-c4ccc(N(c5ccc(-c6ccc(-c7ccc8ccccc8c7)cc6)cc5)c5ccc(-c6cccc7c6oc6ccccc67)cc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc(-c4cccc5c4oc4ccccc45)cc3)c3ccccc23)cc1]

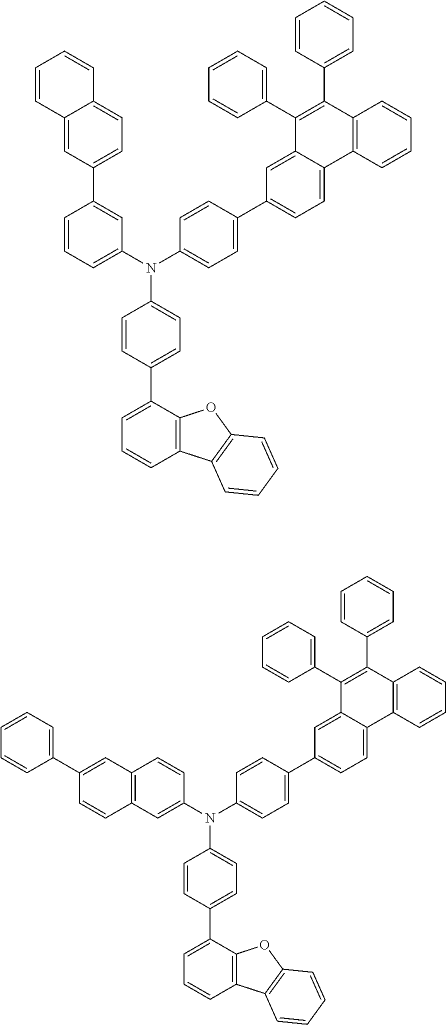 [SMILES: c1ccc(-c2c(-c3ccccc3)c3cc(-c4ccc(N(c5ccc(-c6cccc7c6oc6ccccc67)cc5)c5cccc(-c6ccc7ccccc7c6)c5)cc4)ccc3c3ccccc23)cc1 ; c1ccc(-c2ccc3cc(N(c4ccc(-c5ccc6c(c5)c(-c5ccccc5)c(-c5ccccc5)c5ccccc56)cc4)c4ccc(-c5cccc6c5oc5ccccc56)cc4)ccc3c2)cc1]

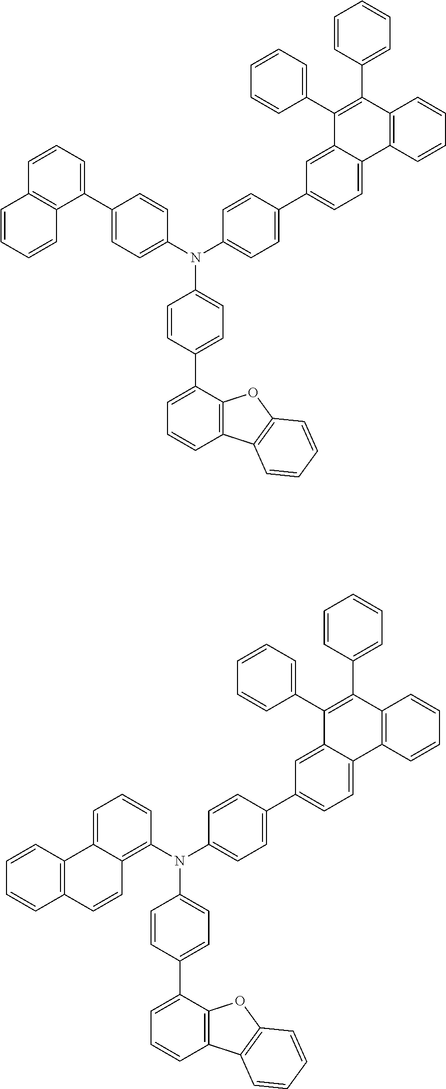 [SMILES: c1ccc(-c2c(-c3ccccc3)c3cc(-c4ccc(N(c5ccc(-c6cccc7ccccc67)cc5)c5ccc(-c6cccc7c6oc6ccccc67)cc5)cc4)ccc3c3ccccc23)cc1 ; c1ccc(-c2c(-c3ccccc3)c3cc(-c4ccc(N(c5ccc(-c6cccc7c6oc6ccccc67)cc5)c5cccc6c5ccc5ccccc56)cc4)ccc3c3ccccc23)cc1]

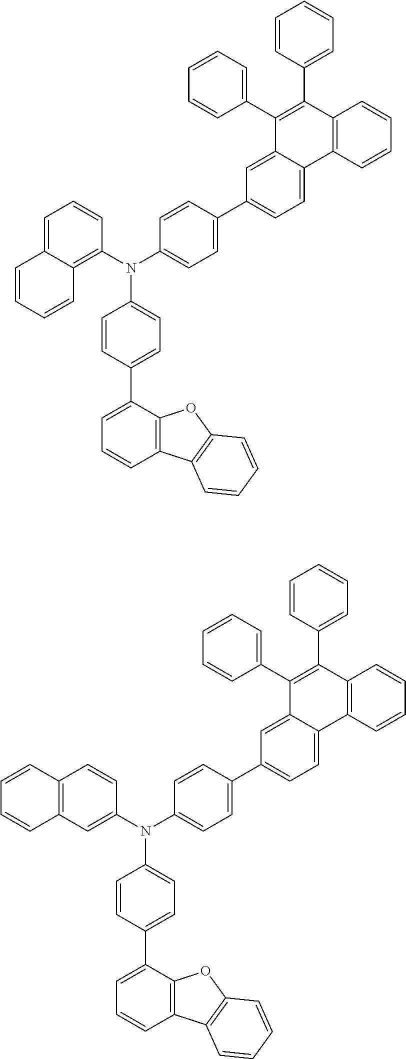 [SMILES: c1ccc(-c2c(-c3ccccc3)c3cc(-c4ccc(N(c5ccc(-c6cccc7c6oc6ccccc67)cc5)c5cccc6ccccc56)cc4)ccc3c3ccccc23)cc1 ; c1ccc(-c2c(-c3ccccc3)c3cc(-c4ccc(N(c5ccc(-c6cccc7c6oc6ccccc67)cc5)c5ccc6ccccc6c5)cc4)ccc3c3ccccc23)cc1]

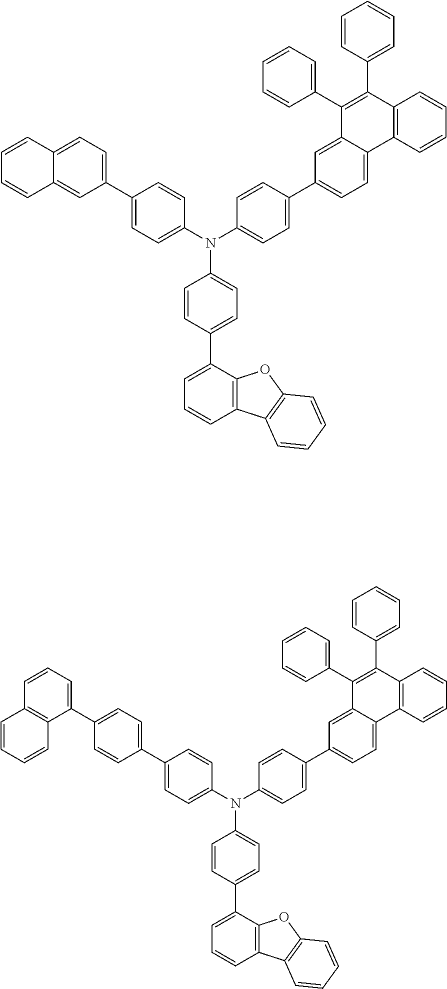 [SMILES: c1ccc(-c2c(-c3ccccc3)c3cc(-c4ccc(N(c5ccc(-c6ccc7ccccc7c6)cc5)c5ccc(-c6cccc7c6oc6ccccc67)cc5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc(-c6cccc7c6oc6ccccc67)cc5)cc4)ccc3c3ccccc23)cc1]

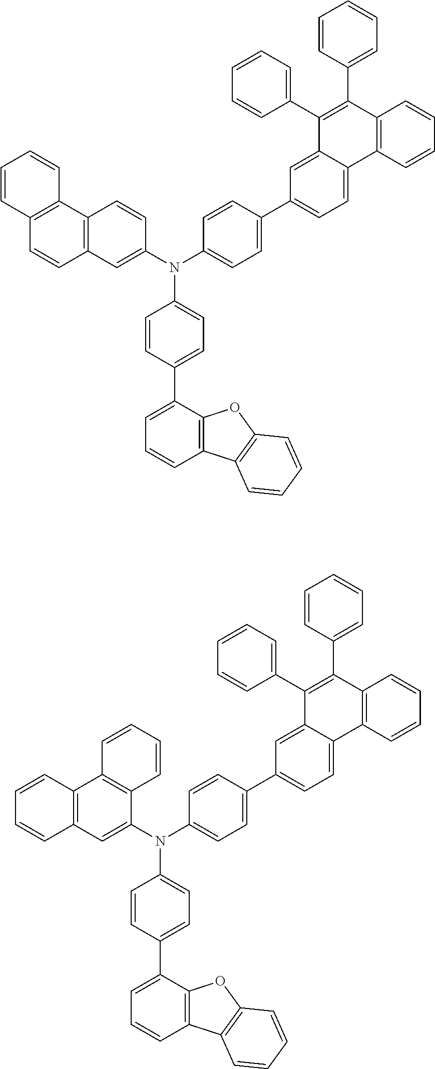 [SMILES: c1ccc(-c2c(-c3ccccc3)c3cc(-c4ccc(N(c5ccc(-c6cccc7c6oc6ccccc67)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6cccc7c6oc6ccccc67)cc5)c5cc6ccccc6c6ccccc56)cc4)ccc3c3ccccc23)cc1]

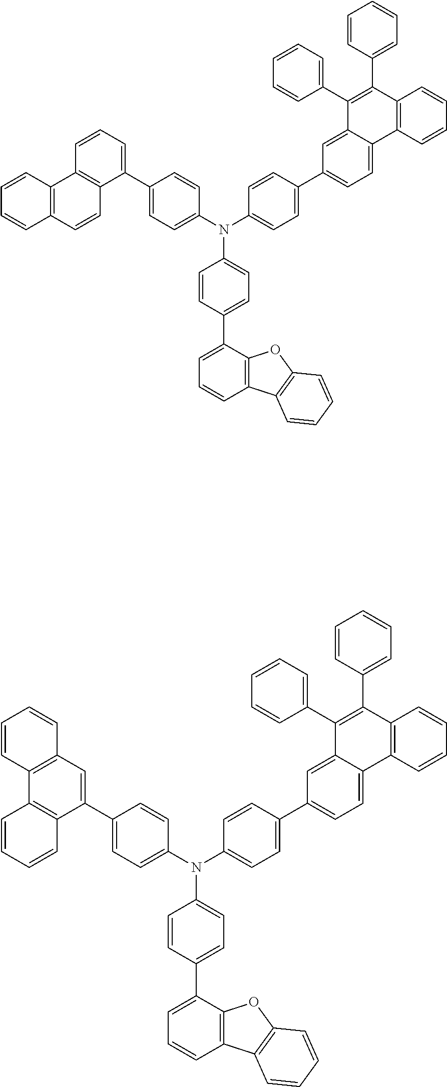 [SMILES: c1ccc(-c2c(-c3ccccc3)c3cc(-c4ccc(N(c5ccc(-c6cccc7c6ccc6ccccc67)cc5)c5ccc(-c6cccc7c6oc6ccccc67)cc5)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc(-c6cccc7c6oc6ccccc67)cc5)cc4)ccc3c3ccccc23)cc1]

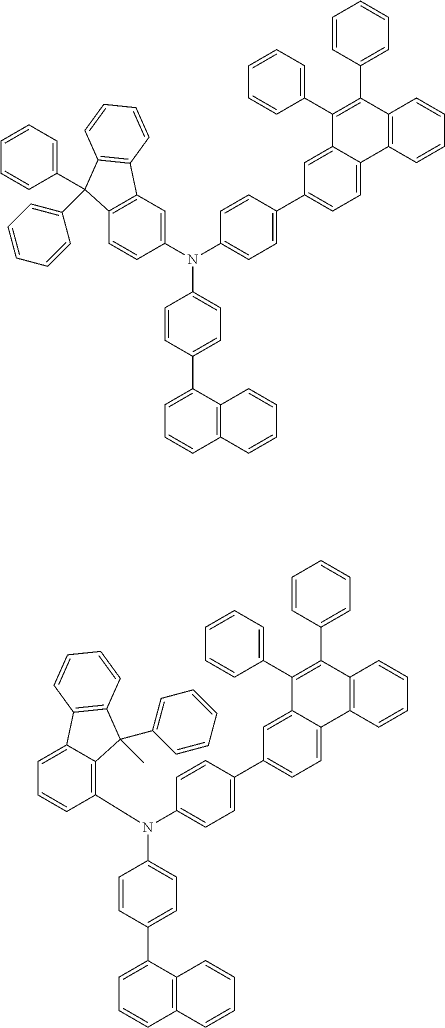 [SMILES: c1ccc(-c2c(-c3ccccc3)c3cc(-c4ccc(N(c5ccc(-c6cccc7ccccc67)cc5)c5ccc6c(c5)-c5ccccc5C6(c5ccccc5)c5ccccc5)cc4)ccc3c3ccccc23)cc1 ; CC1(c2ccccc2)c2ccccc2-c2cccc(N(c3ccc(-c4ccc5c(c4)c(-c4ccccc4)c(-c4ccccc4)c4ccccc45)cc3)c3ccc(-c4cccc5ccccc45)cc3)c21]

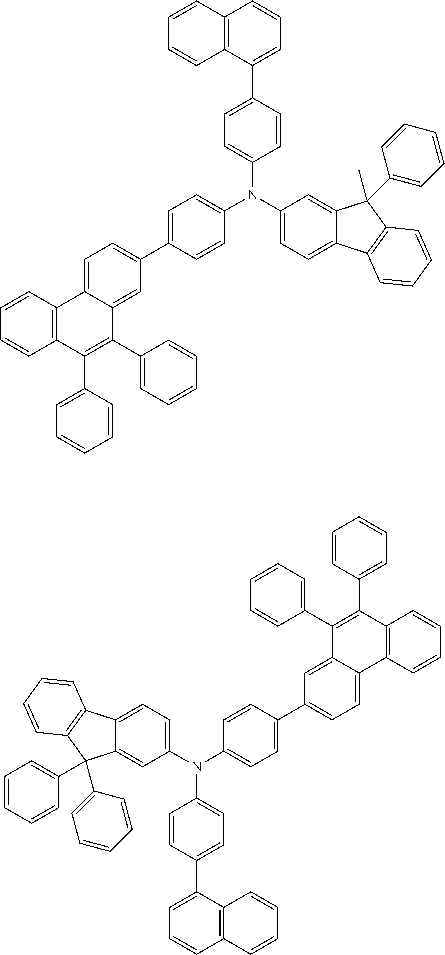 [SMILES: CC1(c2ccccc2)c2ccccc2-c2ccc(N(c3ccc(-c4ccc5c(c4)c(-c4ccccc4)c(-c4ccccc4)c4ccccc45)cc3)c3ccc(-c4cccc5ccccc45)cc3)cc21 ; c1ccc(-c2c(-c3ccccc3)c3cc(-c4ccc(N(c5ccc(-c6cccc7ccccc67)cc5)c5ccc6c(c5)C(c5ccccc5)(c5ccccc5)c5ccccc5-6)cc4)ccc3c3ccccc23)cc1]

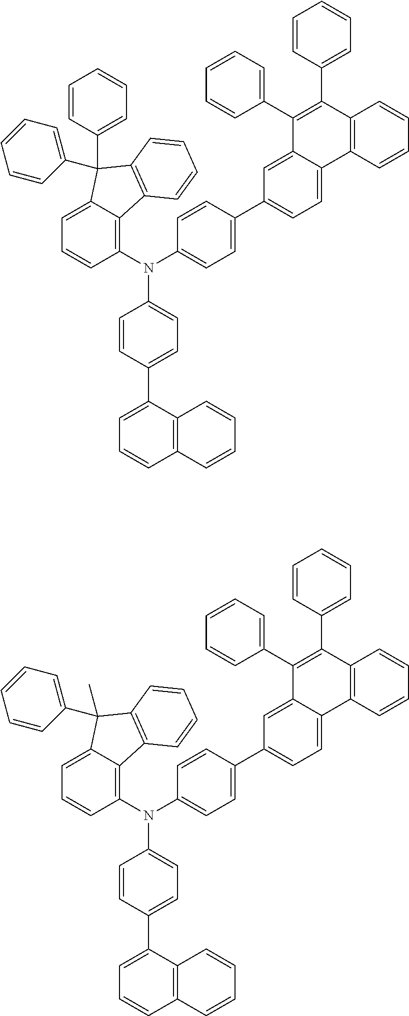 [SMILES: c1ccc(-c2c(-c3ccccc3)c3cc(-c4ccc(N(c5ccc(-c6cccc7ccccc67)cc5)c5cccc6c5-c5ccccc5C6(c5ccccc5)c5ccccc5)cc4)ccc3c3ccccc23)cc1 ; CC1(c2ccccc2)c2ccccc2-c2c(N(c3ccc(-c4ccc5c(c4)c(-c4ccccc4)c(-c4ccccc4)c4ccccc45)cc3)c3ccc(-c4cccc5ccccc45)cc3)cccc21]

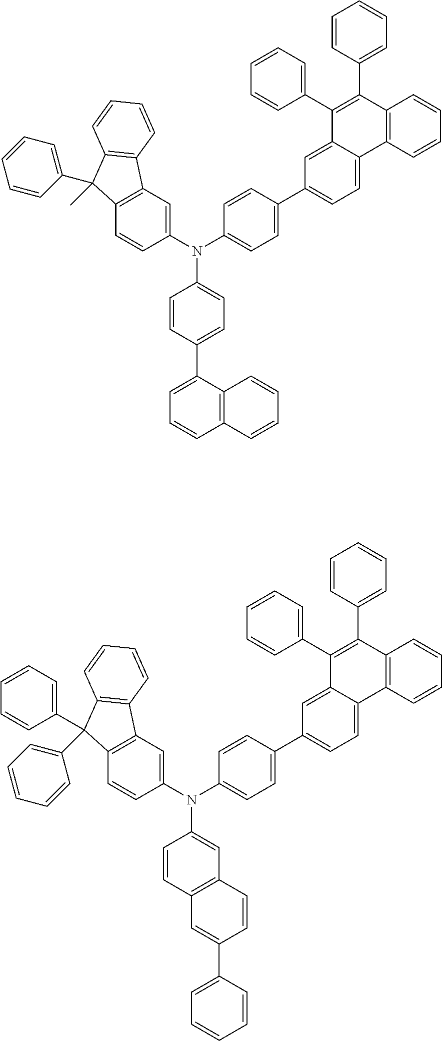 [SMILES: CC1(c2ccccc2)c2ccccc2-c2cc(N(c3ccc(-c4ccc5c(c4)c(-c4ccccc4)c(-c4ccccc4)c4ccccc45)cc3)c3ccc(-c4cccc5ccccc45)cc3)ccc21 ; c1ccc(-c2ccc3cc(N(c4ccc(-c5ccc6c(c5)c(-c5ccccc5)c(-c5ccccc5)c5ccccc56)cc4)c4ccc5c(c4)-c4ccccc4C5(c4ccccc4)c4ccccc4)ccc3c2)cc1]

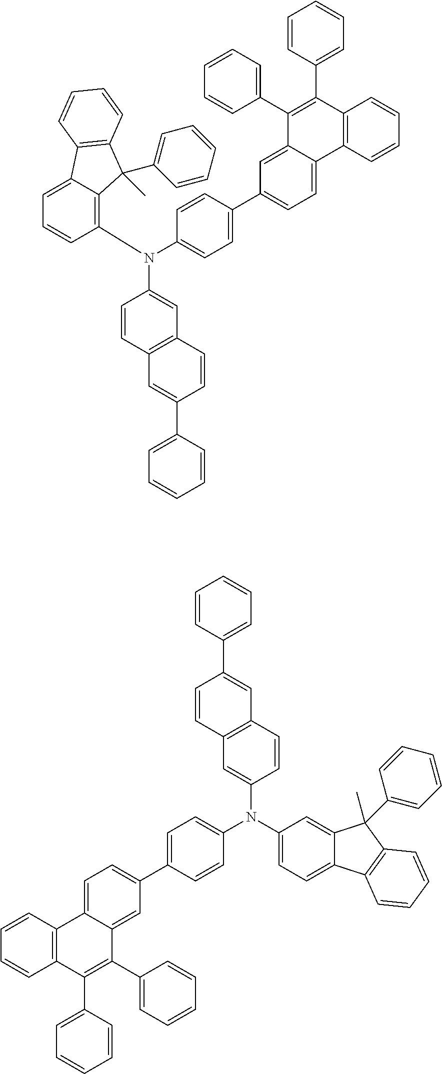 [SMILES: CC1(c2ccccc2)c2ccccc2-c2cccc(N(c3ccc(-c4ccc5c(c4)c(-c4ccccc4)c(-c4ccccc4)c4ccccc45)cc3)c3ccc4cc(-c5ccccc5)ccc4c3)c21 ; CC1(c2ccccc2)c2ccccc2-c2ccc(N(c3ccc(-c4ccc5c(c4)c(-c4ccccc4)c(-c4ccccc4)c4ccccc45)cc3)c3ccc4cc(-c5ccccc5)ccc4c3)cc21]

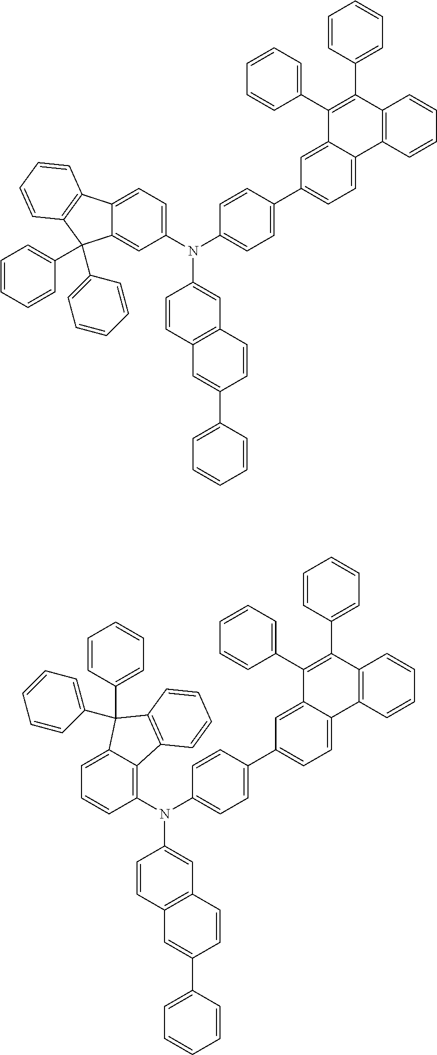 [SMILES: c1ccc(-c2ccc3cc(N(c4ccc(-c5ccc6c(c5)c(-c5ccccc5)c(-c5ccccc5)c5ccccc56)cc4)c4ccc5c(c4)C(c4ccccc4)(c4ccccc4)c4ccccc4-5)ccc3c2)cc1 ; c1ccc(-c2ccc3cc(N(c4ccc(-c5ccc6c(c5)c(-c5ccccc5)c(-c5ccccc5)c5ccccc56)cc4)c4cccc5c4-c4ccccc4C5(c4ccccc4)c4ccccc4)ccc3c2)cc1]

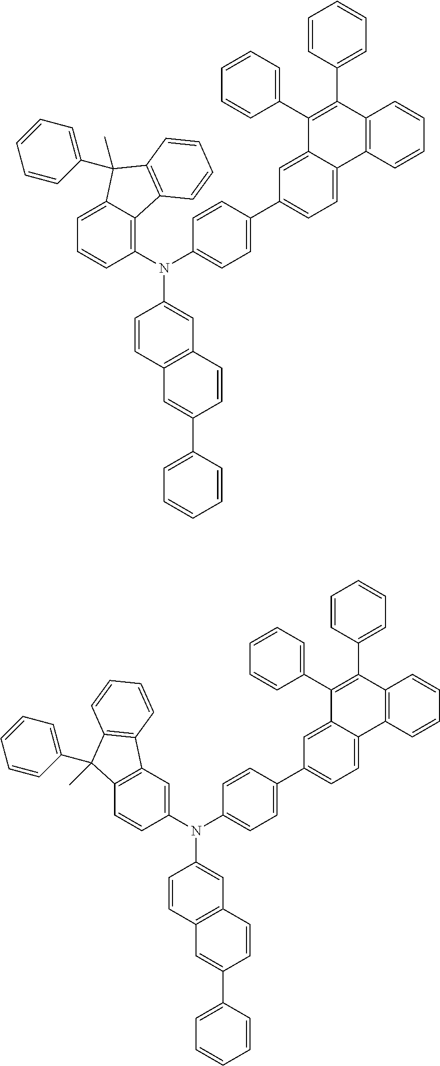 [SMILES: CC1(c2ccccc2)c2ccccc2-c2c(N(c3ccc(-c4ccc5c(c4)c(-c4ccccc4)c(-c4ccccc4)c4ccccc45)cc3)c3ccc4cc(-c5ccccc5)ccc4c3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3ccc4cc(-c5ccccc5)ccc4c3)ccc21]

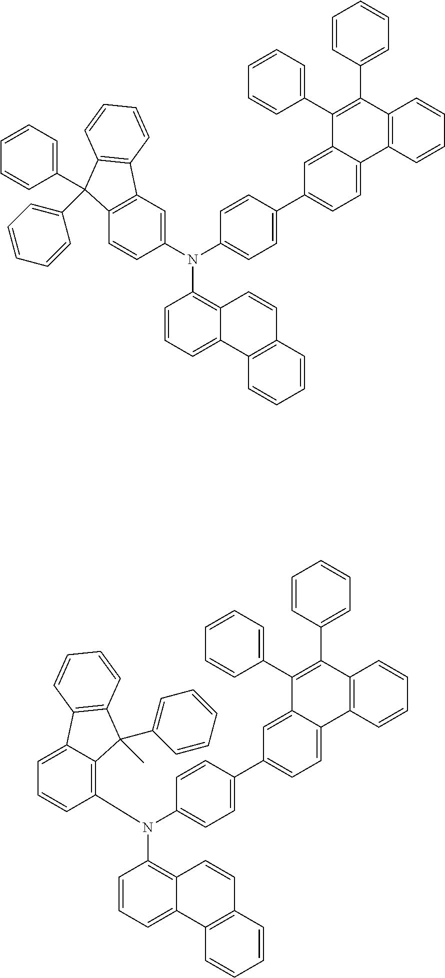 [SMILES: c1ccc(-c2c(-c3ccccc3)c3cc(-c4ccc(N(c5ccc6c(c5)-c5ccccc5C6(c5ccccc5)c5ccccc5)c5cccc6c5ccc5ccccc56)cc4)ccc3c3ccccc23)cc1 ; CC1(c2ccccc2)c2ccccc2-c2cccc(N(c3ccc(-c4ccc5c(c4)c(-c4ccccc4)c(-c4ccccc4)c4ccccc45)cc3)c3cccc4c3ccc3ccccc34)c21]

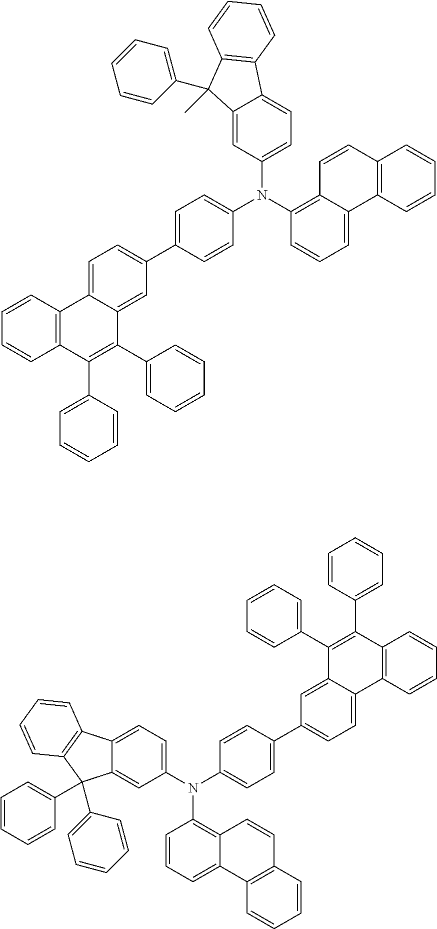 [SMILES: CC1(c2ccccc2)c2ccccc2-c2ccc(N(c3ccc(-c4ccc5c(c4)c(-c4ccccc4)c(-c4ccccc4)c4ccccc45)cc3)c3cccc4c3ccc3ccccc34)cc21 ; c1ccc(-c2c(-c3ccccc3)c3cc(-c4ccc(N(c5ccc6c(c5)C(c5ccccc5)(c5ccccc5)c5ccccc5-6)c5cccc6c5ccc5ccccc56)cc4)ccc3c3ccccc23)cc1]

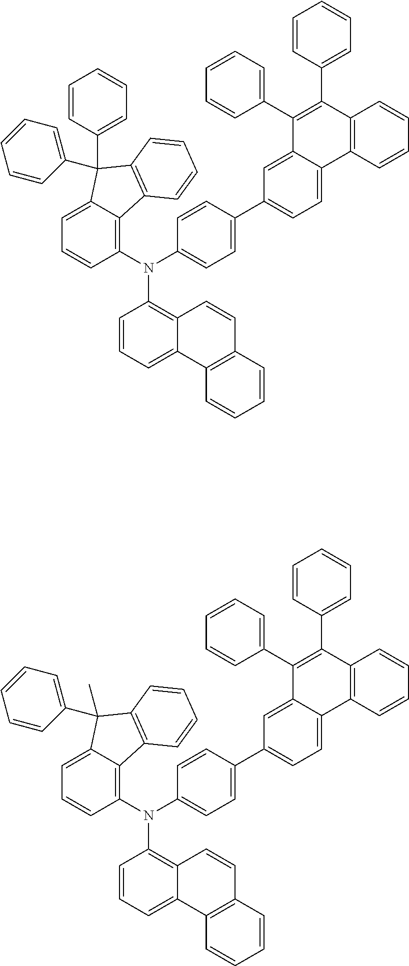 [SMILES: c1ccc(-c2c(-c3ccccc3)c3cc(-c4ccc(N(c5cccc6c5-c5ccccc5C6(c5ccccc5)c5ccccc5)c5cccc6c5ccc5ccccc56)cc4)ccc3c3ccccc23)cc1 ; CC1(c2ccccc2)c2ccccc2-c2c(N(c3ccc(-c4ccc5c(c4)c(-c4ccccc4)c(-c4ccccc4)c4ccccc45)cc3)c3cccc4c3ccc3ccccc34)cccc21]

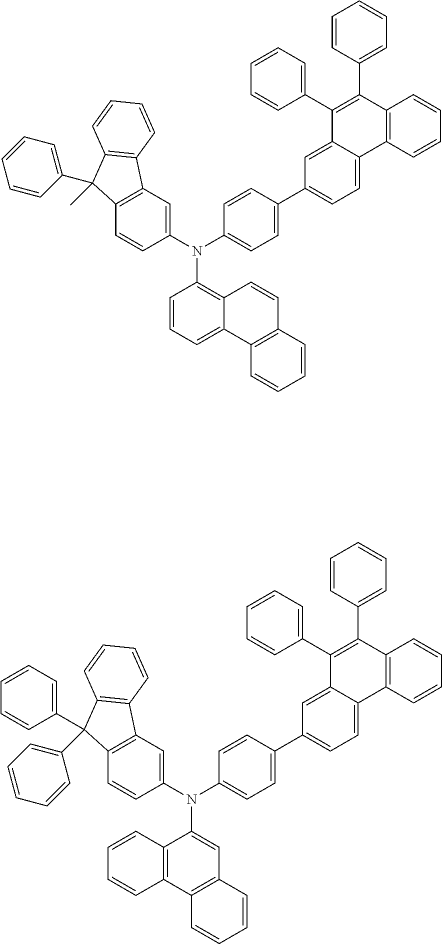 [SMILES: CC1(c2ccccc2)c2ccccc2-c2cc(N(c3ccc(-c4ccc5c(c4)c(-c4ccccc4)c(-c4ccccc4)c4ccccc45)cc3)c3cccc4c3ccc3ccccc34)ccc21 ; c1ccc(-c2c(-c3ccccc3)c3cc(-c4ccc(N(c5ccc6c(c5)-c5ccccc5C6(c5ccccc5)c5ccccc5)c5cc6ccccc6c6ccccc56)cc4)ccc3c3ccccc23)cc1]

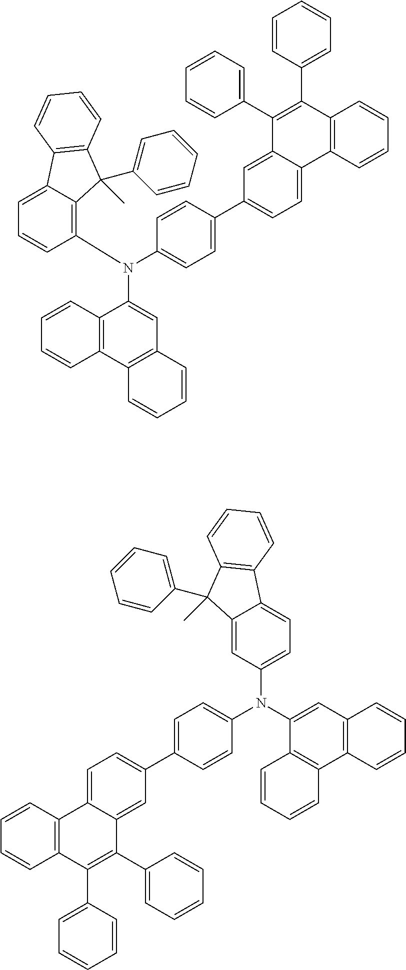 [SMILES: CC1(c2ccccc2)c2ccccc2-c2cccc(N(c3ccc(-c4ccc5c(c4)c(-c4ccccc4)c(-c4ccccc4)c4ccccc45)cc3)c3cc4ccccc4c4ccccc34)c21 ; CC1(c2ccccc2)c2ccccc2-c2ccc(N(c3ccc(-c4ccc5c(c4)c(-c4ccccc4)c(-c4ccccc4)c4ccccc45)cc3)c3cc4ccccc4c4ccccc34)cc21]

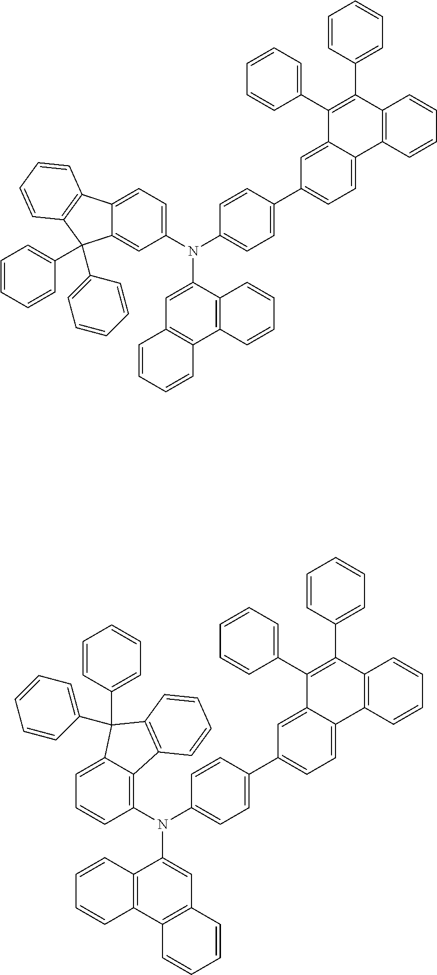 [SMILES: c1ccc(-c2c(-c3ccccc3)c3cc(-c4ccc(N(c5ccc6c(c5)C(c5ccccc5)(c5ccccc5)c5ccccc5-6)c5cc6ccccc6c6ccccc56)cc4)ccc3c3ccccc23)cc1 ; c1ccc(-c2c(-c3ccccc3)c3cc(-c4ccc(N(c5cccc6c5-c5ccccc5C6(c5ccccc5)c5ccccc5)c5cc6ccccc6c6ccccc56)cc4)ccc3c3ccccc23)cc1]

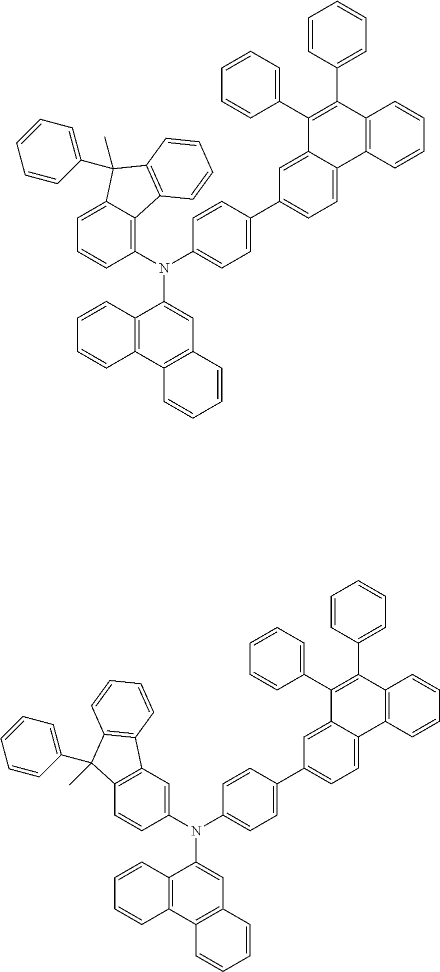 [SMILES: CC1(c2ccccc2)c2ccccc2-c2c(N(c3ccc(-c4ccc5c(c4)c(-c4ccccc4)c(-c4ccccc4)c4ccccc45)cc3)c3cc4ccccc4c4ccccc34)cccc21 ; CC1(c2ccccc2)c2ccccc2-c2cc(N(c3ccc(-c4ccc5c(c4)c(-c4ccccc4)c(-c4ccccc4)c4ccccc45)cc3)c3cc4ccccc4c4ccccc34)ccc21]

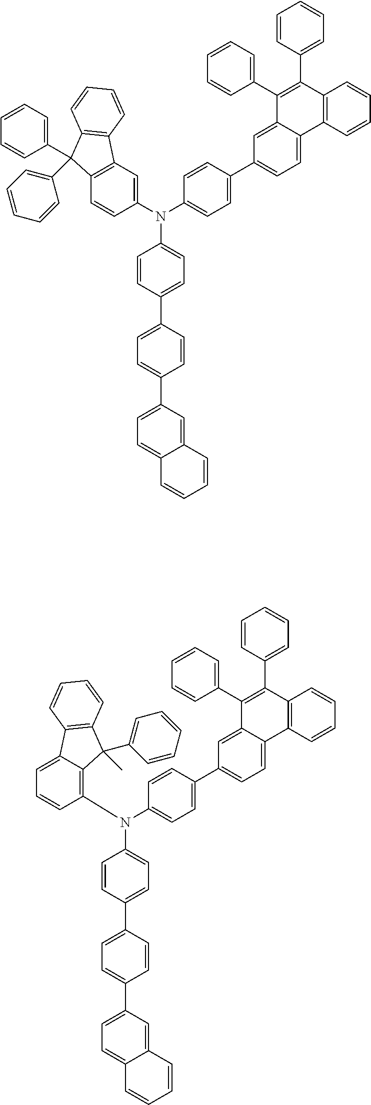 [SMILES: c1ccc(-c2c(-c3ccccc3)c3cc(-c4ccc(N(c5ccc(-c6ccc(-c7ccc8ccccc8c7)cc6)cc5)c5ccc6c(c5)-c5ccccc5C6(c5ccccc5)c5ccccc5)cc4)ccc3c3ccccc23)cc1 ; CC1(c2ccccc2)c2ccccc2-c2cccc(N(c3ccc(-c4ccc(-c5ccc6ccccc6c5)cc4)cc3)c3ccc(-c4ccc5c(c4)c(-c4ccccc4)c(-c4ccccc4)c4ccccc45)cc3)c21]

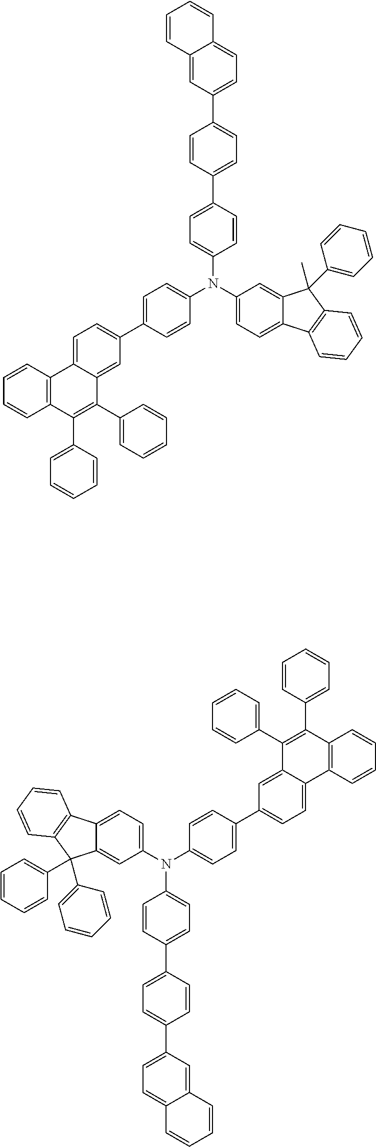 [SMILES: CC1(c2ccccc2)c2ccccc2-c2ccc(N(c3ccc(-c4ccc(-c5ccc6ccccc6c5)cc4)cc3)c3ccc(-c4ccc5c(c4)c(-c4ccccc4)c(-c4ccccc4)c4ccccc45)cc3)cc21 ; c1ccc(-c2c(-c3ccccc3)c3cc(-c4ccc(N(c5ccc(-c6ccc(-c7ccc8ccccc8c7)cc6)cc5)c5ccc6c(c5)C(c5ccccc5)(c5ccccc5)c5ccccc5-6)cc4)ccc3c3ccccc23)cc1]

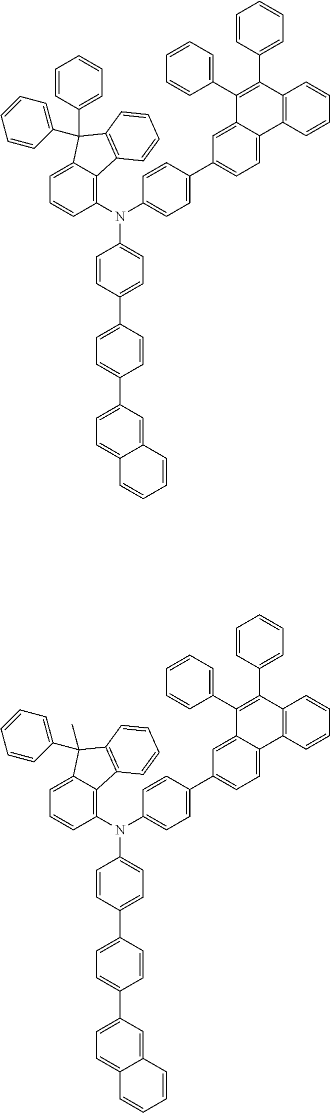 [SMILES: c1ccc(-c2c(-c3ccccc3)c3cc(-c4ccc(N(c5ccc(-c6ccc(-c7ccc8ccccc8c7)cc6)cc5)c5cccc6c5-c5ccccc5C6(c5ccccc5)c5ccccc5)cc4)ccc3c3ccccc23)cc1 ; CC1(c2ccccc2)c2ccccc2-c2c(N(c3ccc(-c4ccc(-c5ccc6ccccc6c5)cc4)cc3)c3ccc(-c4ccc5c(c4)c(-c4ccccc4)c(-c4ccccc4)c4ccccc45)cc3)cccc21]

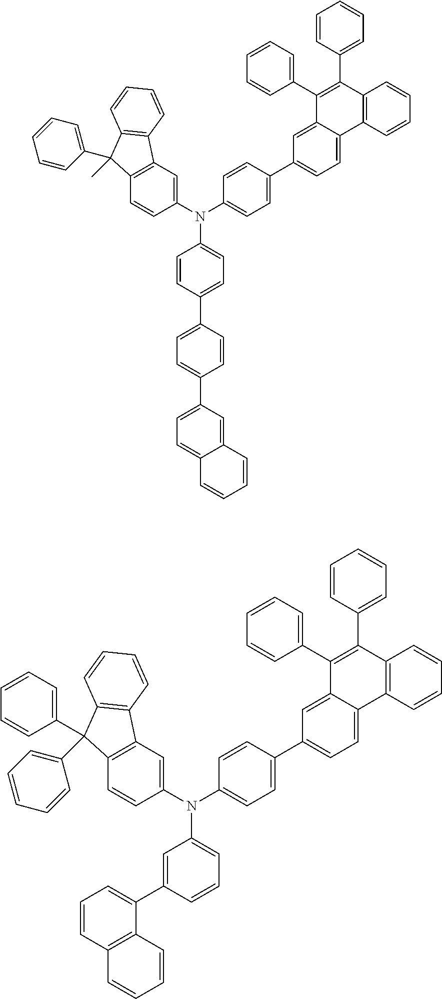 [SMILES: CC1(c2ccccc2)c2ccccc2-c2cc(N(c3ccc(-c4ccc(-c5ccc6ccccc6c5)cc4)cc3)c3ccc(-c4ccc5c(c4)c(-c4ccccc4)c(-c4ccccc4)c4ccccc45)cc3)ccc21 ; c1ccc(-c2c(-c3ccccc3)c3cc(-c4ccc(N(c5cccc(-c6cccc7ccccc67)c5)c5ccc6c(c5)-c5ccccc5C6(c5ccccc5)c5ccccc5)cc4)ccc3c3ccccc23)cc1]

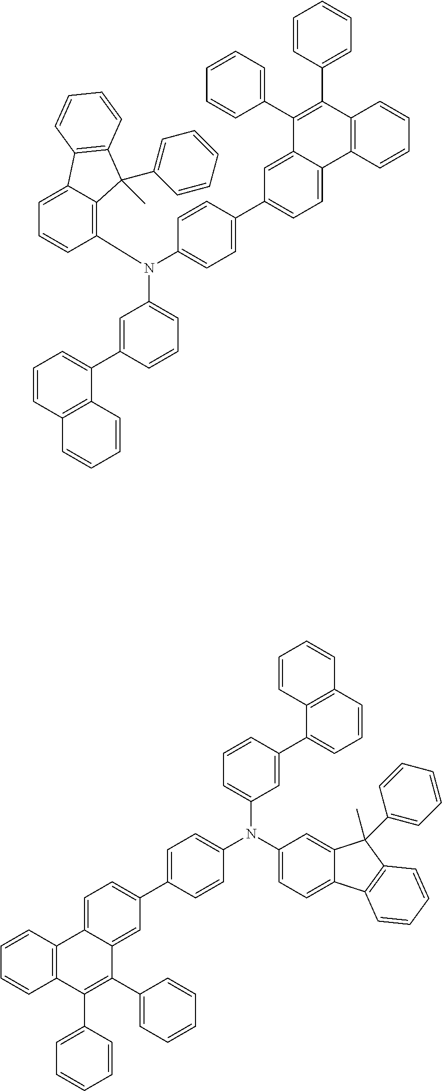 [SMILES: CC1(c2ccccc2)c2ccccc2-c2cccc(N(c3ccc(-c4ccc5c(c4)c(-c4ccccc4)c(-c4ccccc4)c4ccccc45)cc3)c3cccc(-c4cccc5ccccc45)c3)c21 ; CC1(c2ccccc2)c2ccccc2-c2ccc(N(c3ccc(-c4ccc5c(c4)c(-c4ccccc4)c(-c4ccccc4)c4ccccc45)cc3)c3cccc(-c4cccc5ccccc45)c3)cc21]

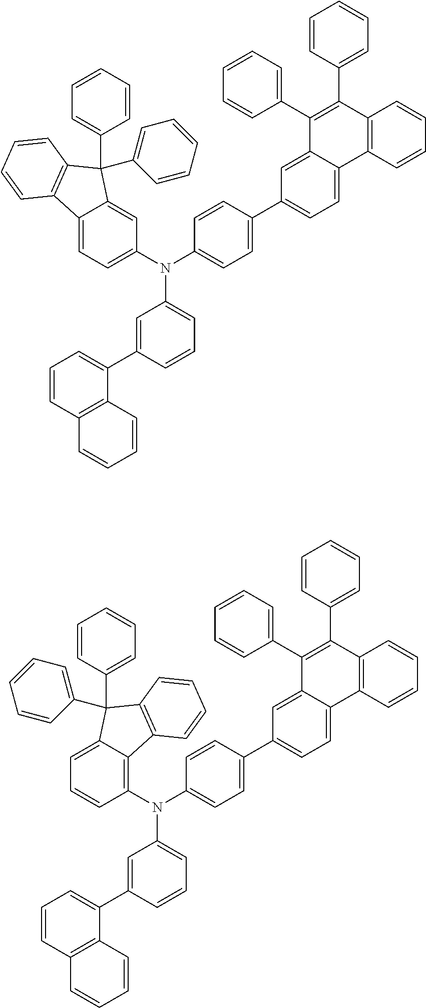 [SMILES: c1ccc(-c2c(-c3ccccc3)c3cc(-c4ccc(N(c5cccc(-c6cccc7ccccc67)c5)c5ccc6c(c5)C(c5ccccc5)(c5ccccc5)c5ccccc5-6)cc4)ccc3c3ccccc23)cc1 ; c1ccc(-c2c(-c3ccccc3)c3cc(-c4ccc(N(c5cccc(-c6cccc7ccccc67)c5)c5cccc6c5-c5ccccc5C6(c5ccccc5)c5ccccc5)cc4)ccc3c3ccccc23)cc1]

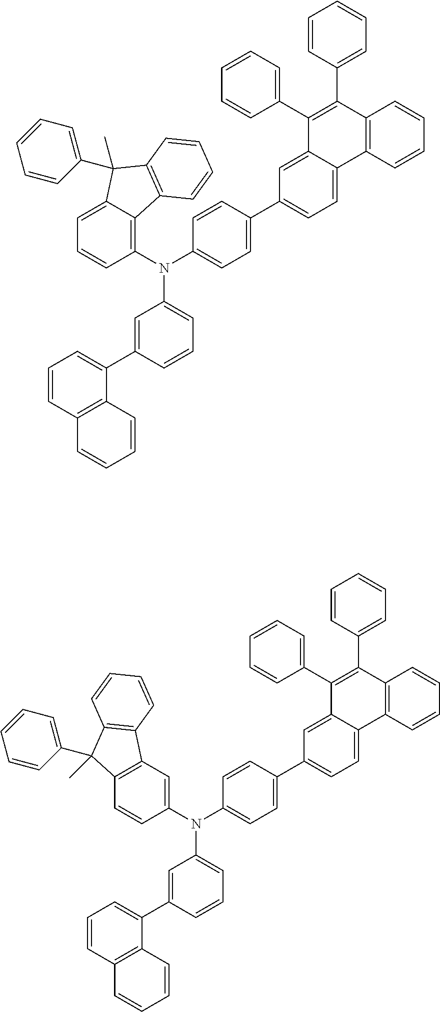 [SMILES: CC1(c2ccccc2)c2ccccc2-c2c(N(c3ccc(-c4ccc5c(c4)c(-c4ccccc4)c(-c4ccccc4)c4ccccc45)cc3)c3cccc(-c4cccc5ccccc45)c3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3cccc(-c4cccc5ccccc45)c3)ccc21]

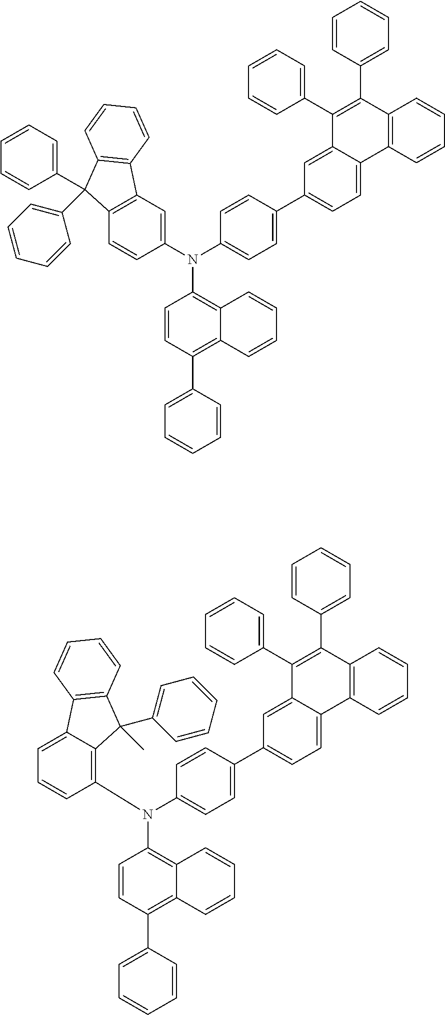 [SMILES: c1ccc(-c2ccc(N(c3ccc(-c4ccc5c(c4)c(-c4ccccc4)c(-c4ccccc4)c4ccccc45)cc3)c3ccc4c(c3)-c3ccccc3C4(c3ccccc3)c3ccccc3)c3ccccc23)cc1 ; CC1(c2ccccc2)c2ccccc2-c2cccc(N(c3ccc(-c4ccc5c(c4)c(-c4ccccc4)c(-c4ccccc4)c4ccccc45)cc3)c3ccc(-c4ccccc4)c4ccccc34)c21]

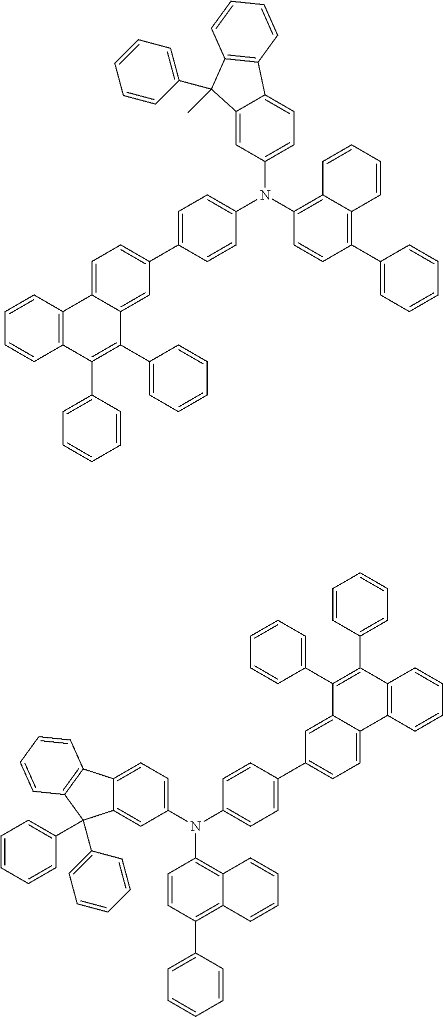 [SMILES: CC1(c2ccccc2)c2ccccc2-c2ccc(N(c3ccc(-c4ccc5c(c4)c(-c4ccccc4)c(-c4ccccc4)c4ccccc45)cc3)c3ccc(-c4ccccc4)c4ccccc34)cc21 ; c1ccc(-c2ccc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)c3ccccc23)cc1]

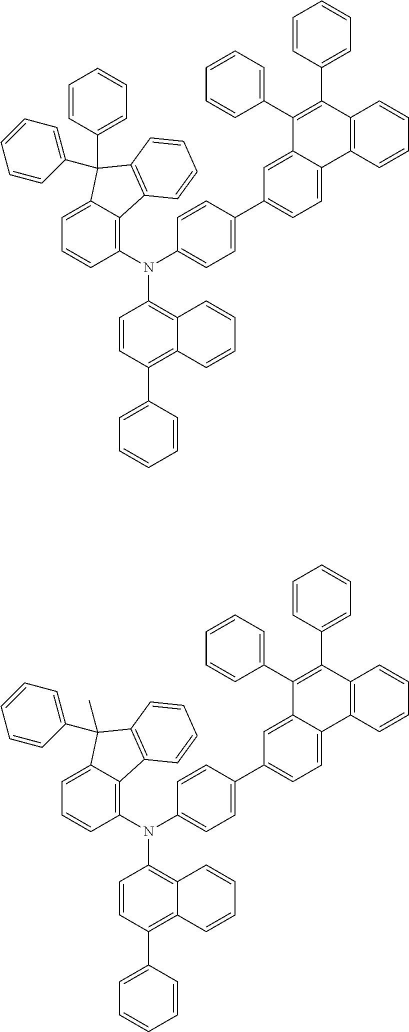 [SMILES: c1ccc(-c2ccc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)c3ccccc23)cc1 ; CC1(c2ccccc2)c2ccccc2-c2c(N(c3ccc(-c4ccc5c(c4)c(-c4ccccc4)c(-c4ccccc4)c4ccccc45)cc3)c3ccc(-c4ccccc4)c4ccccc34)cccc21]

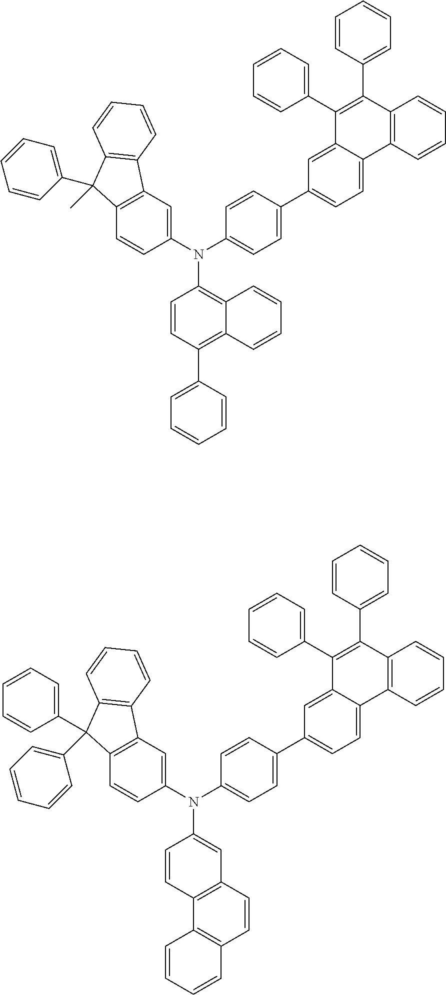 [SMILES: CC1(c2ccccc2)c2ccccc2-c2cc(N(c3ccc(-c4ccc5c(c4)c(-c4ccccc4)c(-c4ccccc4)c4ccccc45)cc3)c3ccc(-c4ccccc4)c4ccccc34)ccc21 ; c1ccc(-c2c(-c3ccccc3)c3cc(-c4ccc(N(c5ccc6c(c5)-c5ccccc5C6(c5ccccc5)c5ccccc5)c5ccc6c(ccc7ccccc76)c5)cc4)ccc3c3ccccc23)cc1]

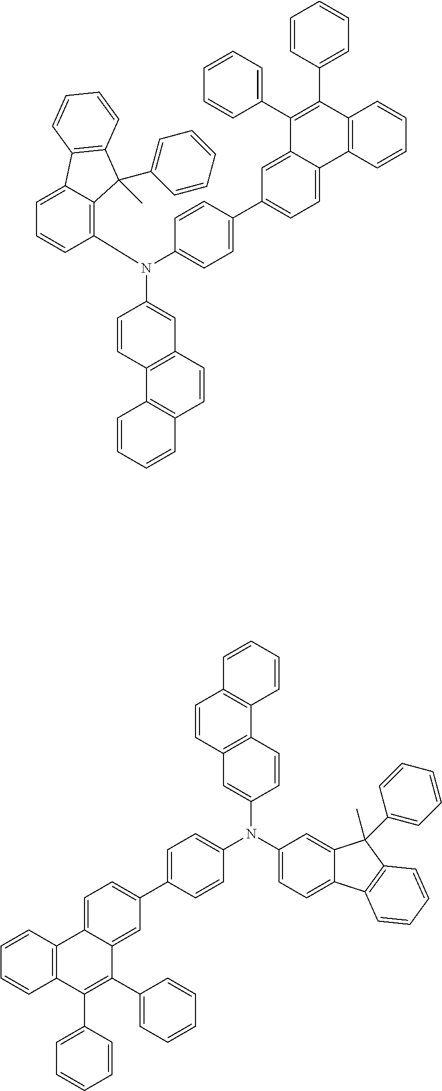 [SMILES: CC1(c2ccccc2)c2ccccc2-c2cccc(N(c3ccc(-c4ccc5c(c4)c(-c4ccccc4)c(-c4ccccc4)c4ccccc45)cc3)c3ccc4c(ccc5ccccc54)c3)c21 ; CC1(c2ccccc2)c2ccccc2-c2ccc(N(c3ccc(-c4ccc5c(c4)c(-c4ccccc4)c(-c4ccccc4)c4ccccc45)cc3)c3ccc4c(ccc5ccccc54)c3)cc21]

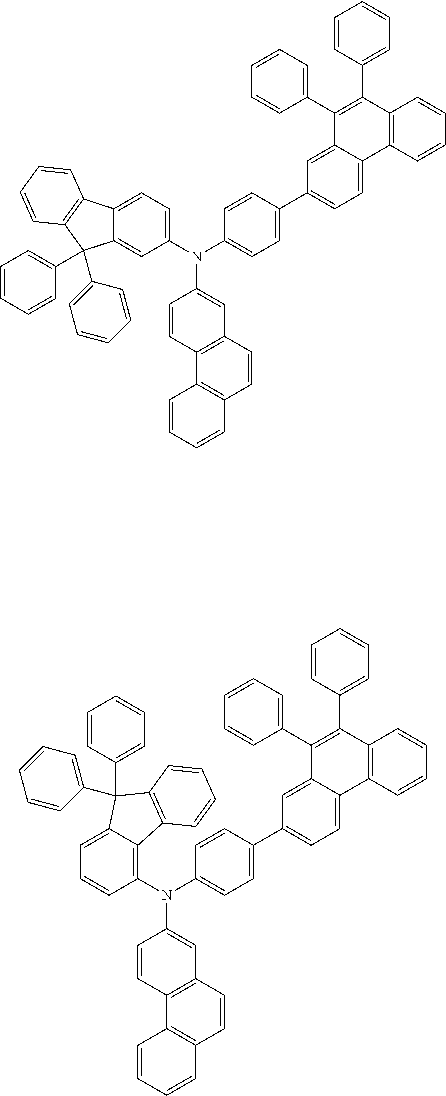 [SMILES: c1ccc(-c2c(-c3ccccc3)c3cc(-c4ccc(N(c5ccc6c(c5)C(c5ccccc5)(c5ccccc5)c5ccccc5-6)c5ccc6c(ccc7ccccc76)c5)cc4)ccc3c3ccccc23)cc1 ; c1ccc(-c2c(-c3ccccc3)c3cc(-c4ccc(N(c5ccc6c(ccc7ccccc76)c5)c5cccc6c5-c5ccccc5C6(c5ccccc5)c5ccccc5)cc4)ccc3c3ccccc23)cc1]

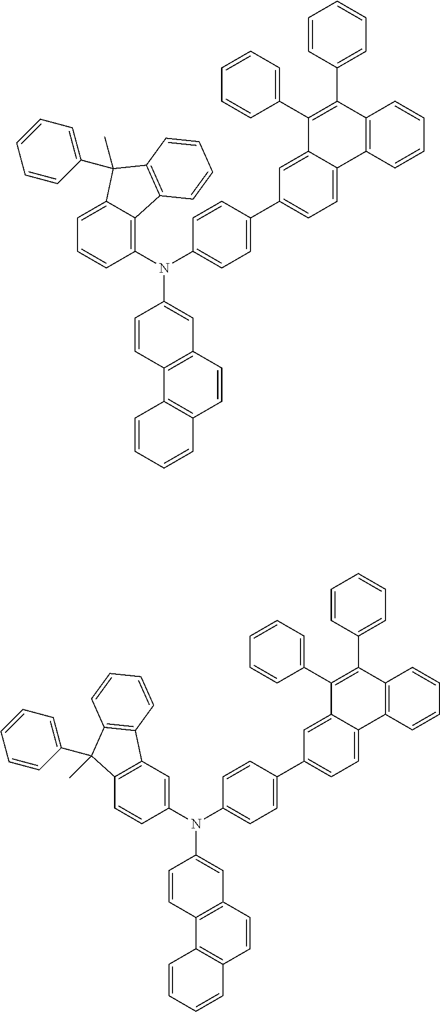 [SMILES: CC1(c2ccccc2)c2ccccc2-c2c(N(c3ccc(-c4ccc5c(c4)c(-c4ccccc4)c(-c4ccccc4)c4ccccc45)cc3)c3ccc4c(ccc5ccccc54)c3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3ccc4c(ccc5ccccc54)c3)ccc21]

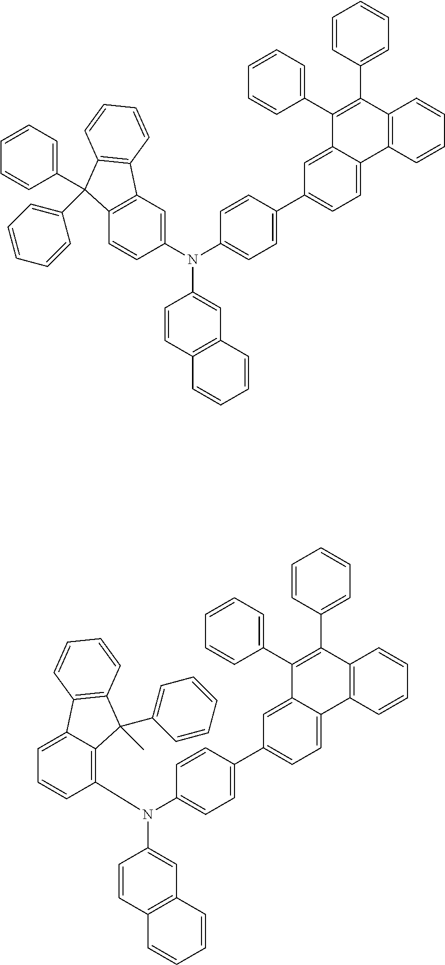 [SMILES: c1ccc(-c2c(-c3ccccc3)c3cc(-c4ccc(N(c5ccc6c(c5)-c5ccccc5C6(c5ccccc5)c5ccccc5)c5ccc6ccccc6c5)cc4)ccc3c3ccccc23)cc1 ; CC1(c2ccccc2)c2ccccc2-c2cccc(N(c3ccc(-c4ccc5c(c4)c(-c4ccccc4)c(-c4ccccc4)c4ccccc45)cc3)c3ccc4ccccc4c3)c21]

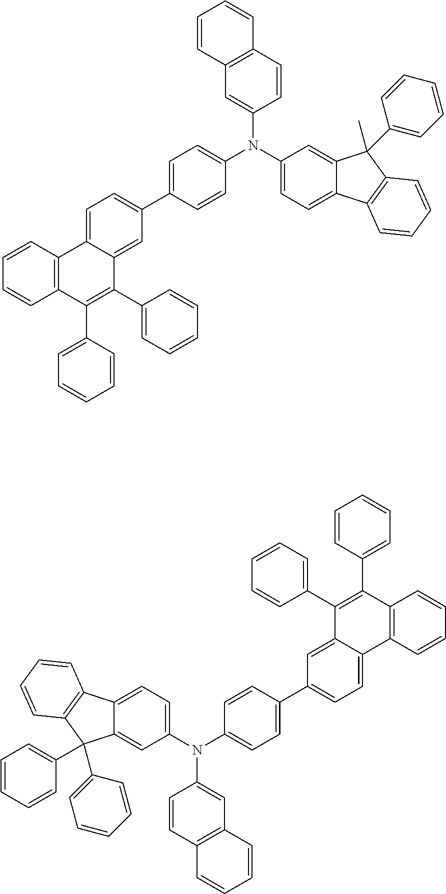 [SMILES: CC1(c2ccccc2)c2ccccc2-c2ccc(N(c3ccc(-c4ccc5c(c4)c(-c4ccccc4)c(-c4ccccc4)c4ccccc45)cc3)c3ccc4ccccc4c3)cc21 ; c1ccc(-c2c(-c3ccccc3)c3cc(-c4ccc(N(c5ccc6c(c5)C(c5ccccc5)(c5ccccc5)c5ccccc5-6)c5ccc6ccccc6c5)cc4)ccc3c3ccccc23)cc1]

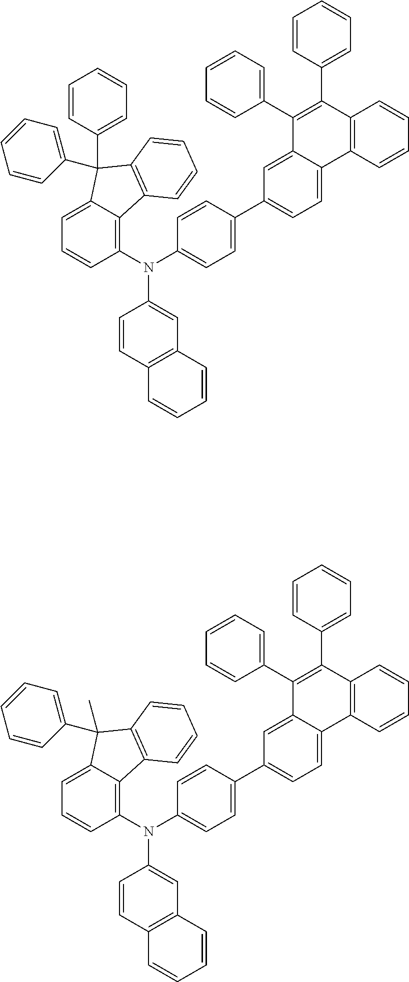 [SMILES: c1ccc(-c2c(-c3ccccc3)c3cc(-c4ccc(N(c5ccc6ccccc6c5)c5cccc6c5-c5ccccc5C6(c5ccccc5)c5ccccc5)cc4)ccc3c3ccccc23)cc1 ; CC1(c2ccccc2)c2ccccc2-c2c(N(c3ccc(-c4ccc5c(c4)c(-c4ccccc4)c(-c4ccccc4)c4ccccc45)cc3)c3ccc4ccccc4c3)cccc21]

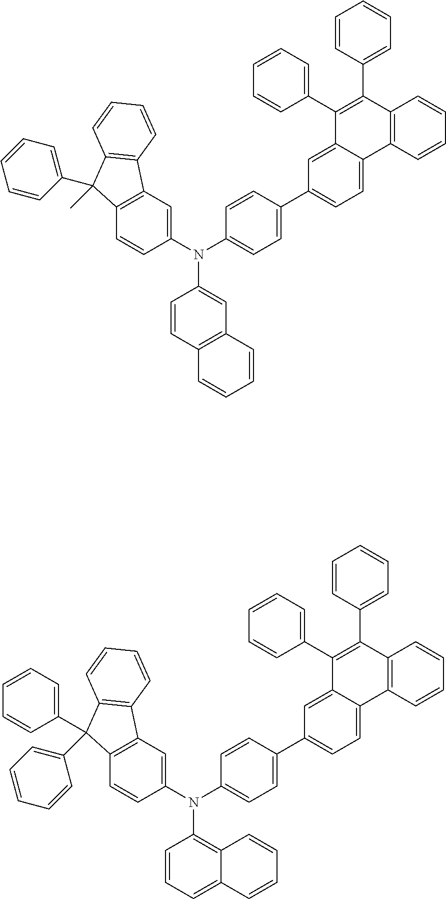 [SMILES: CC1(c2ccccc2)c2ccccc2-c2cc(N(c3ccc(-c4ccc5c(c4)c(-c4ccccc4)c(-c4ccccc4)c4ccccc45)cc3)c3ccc4ccccc4c3)ccc21 ; c1ccc(-c2c(-c3ccccc3)c3cc(-c4ccc(N(c5ccc6c(c5)-c5ccccc5C6(c5ccccc5)c5ccccc5)c5cccc6ccccc56)cc4)ccc3c3ccccc23)cc1]

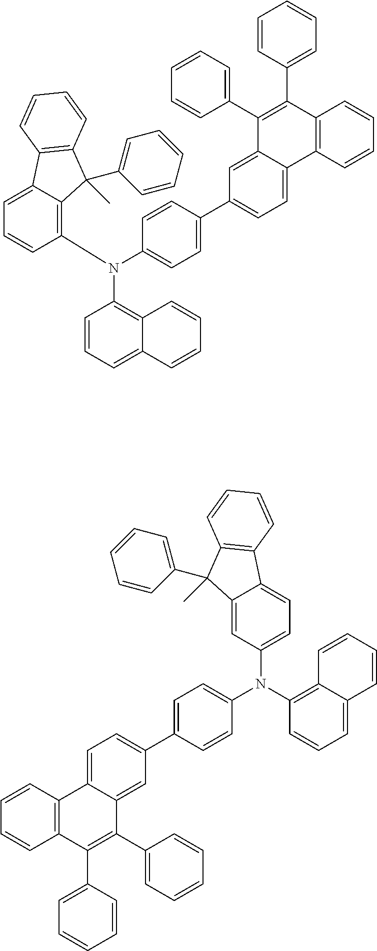 [SMILES: CC1(c2ccccc2)c2ccccc2-c2cccc(N(c3ccc(-c4ccc5c(c4)c(-c4ccccc4)c(-c4ccccc4)c4ccccc45)cc3)c3cccc4ccccc34)c21 ; CC1(c2ccccc2)c2ccccc2-c2ccc(N(c3ccc(-c4ccc5c(c4)c(-c4ccccc4)c(-c4ccccc4)c4ccccc45)cc3)c3cccc4ccccc34)cc21]

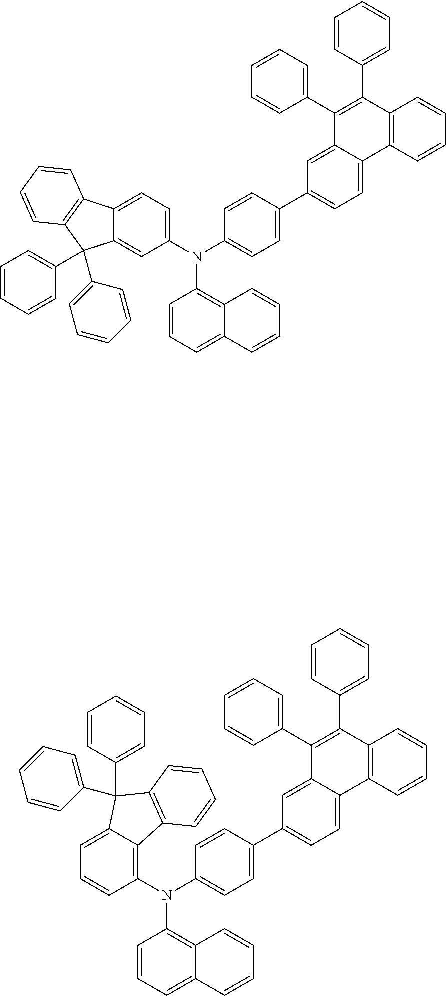 [SMILES: c1ccc(-c2c(-c3ccccc3)c3cc(-c4ccc(N(c5ccc6c(c5)C(c5ccccc5)(c5ccccc5)c5ccccc5-6)c5cccc6ccccc56)cc4)ccc3c3ccccc23)cc1 ; c1ccc(-c2c(-c3ccccc3)c3cc(-c4ccc(N(c5cccc6c5-c5ccccc5C6(c5ccccc5)c5ccccc5)c5cccc6ccccc56)cc4)ccc3c3ccccc23)cc1]

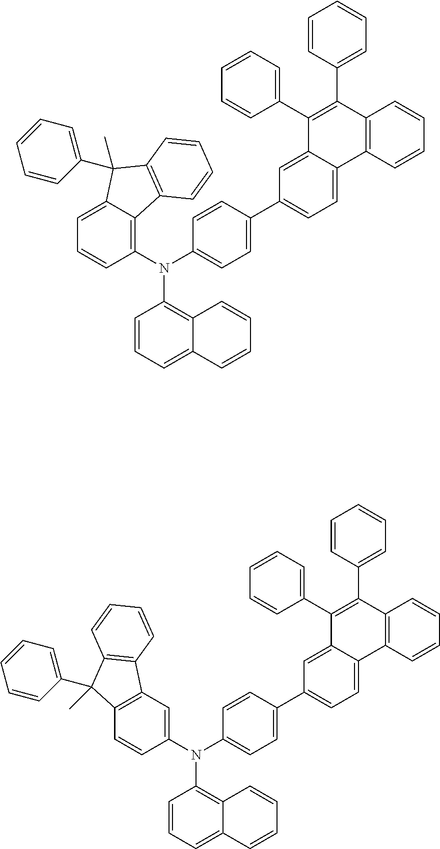 [SMILES: CC1(c2ccccc2)c2ccccc2-c2c(N(c3ccc(-c4ccc5c(c4)c(-c4ccccc4)c(-c4ccccc4)c4ccccc45)cc3)c3cccc4ccccc34)cccc21 ; CC1(c2ccccc2)c2ccccc2-c2cc(N(c3ccc(-c4ccc5c(c4)c(-c4ccccc4)c(-c4ccccc4)c4ccccc45)cc3)c3cccc4ccccc34)ccc21]

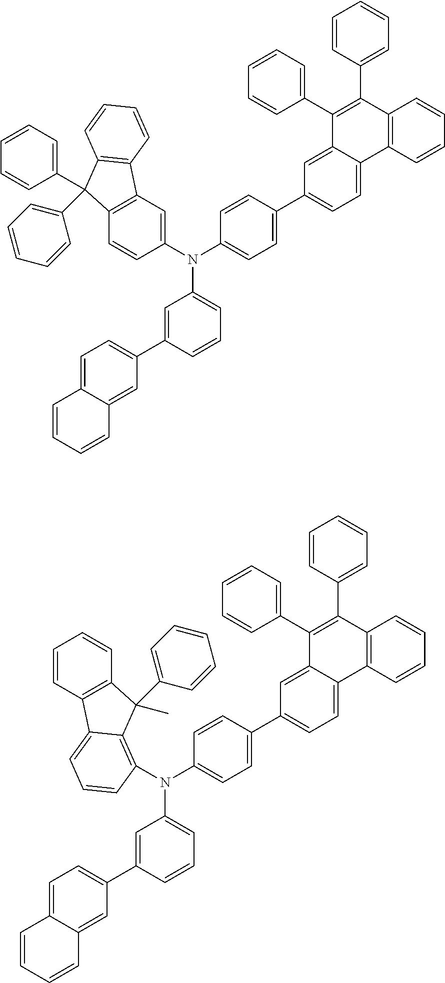 [SMILES: c1ccc(-c2c(-c3ccccc3)c3cc(-c4ccc(N(c5cccc(-c6ccc7ccccc7c6)c5)c5ccc6c(c5)-c5ccccc5C6(c5ccccc5)c5ccccc5)cc4)ccc3c3ccccc23)cc1 ; CC1(c2ccccc2)c2ccccc2-c2cccc(N(c3ccc(-c4ccc5c(c4)c(-c4ccccc4)c(-c4ccccc4)c4ccccc45)cc3)c3cccc(-c4ccc5ccccc5c4)c3)c21]

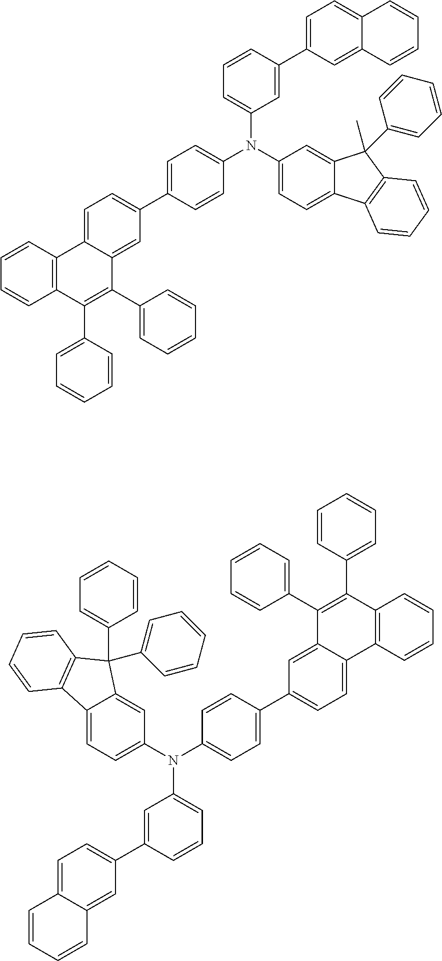 [SMILES: CC1(c2ccccc2)c2ccccc2-c2ccc(N(c3ccc(-c4ccc5c(c4)c(-c4ccccc4)c(-c4ccccc4)c4ccccc45)cc3)c3cccc(-c4ccc5ccccc5c4)c3)cc21 ; c1ccc(-c2c(-c3ccccc3)c3cc(-c4ccc(N(c5cccc(-c6ccc7ccccc7c6)c5)c5ccc6c(c5)C(c5ccccc5)(c5ccccc5)c5ccccc5-6)cc4)ccc3c3ccccc23)cc1]

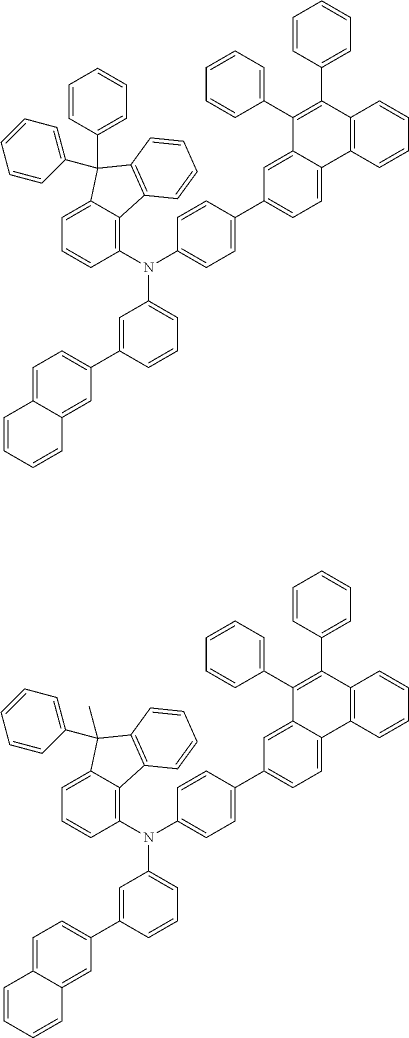 [SMILES: c1ccc(-c2c(-c3ccccc3)c3cc(-c4ccc(N(c5cccc(-c6ccc7ccccc7c6)c5)c5cccc6c5-c5ccccc5C6(c5ccccc5)c5ccccc5)cc4)ccc3c3ccccc23)cc1 ; CC1(c2ccccc2)c2ccccc2-c2c(N(c3ccc(-c4ccc5c(c4)c(-c4ccccc4)c(-c4ccccc4)c4ccccc45)cc3)c3cccc(-c4ccc5ccccc5c4)c3)cccc21]

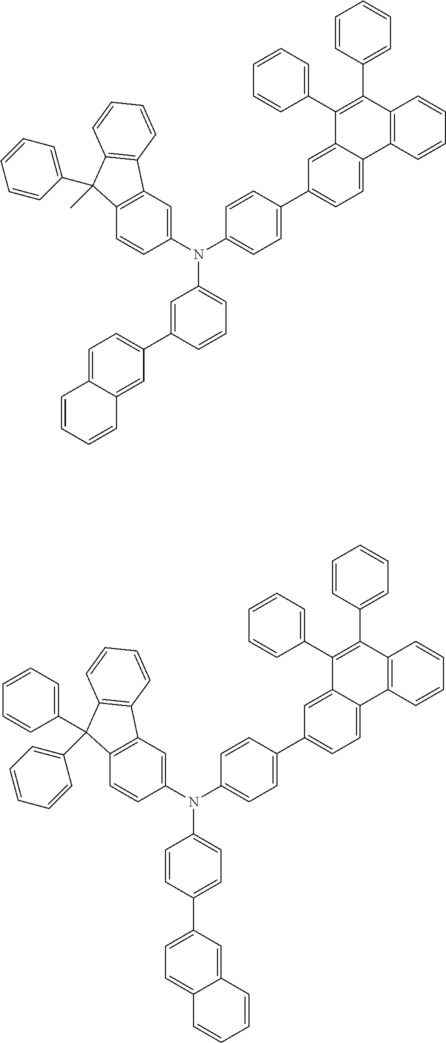 [SMILES: CC1(c2ccccc2)c2ccccc2-c2cc(N(c3ccc(-c4ccc5c(c4)c(-c4ccccc4)c(-c4ccccc4)c4ccccc45)cc3)c3cccc(-c4ccc5ccccc5c4)c3)ccc21 ; c1ccc(-c2c(-c3ccccc3)c3cc(-c4ccc(N(c5ccc(-c6ccc7ccccc7c6)cc5)c5ccc6c(c5)-c5ccccc5C6(c5ccccc5)c5ccccc5)cc4)ccc3c3ccccc23)cc1]

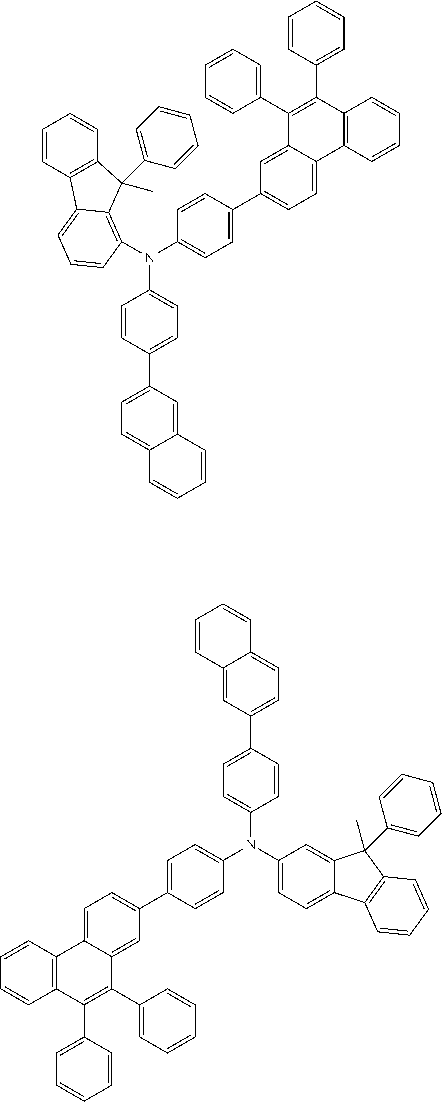 [SMILES: CC1(c2ccccc2)c2ccccc2-c2cccc(N(c3ccc(-c4ccc5ccccc5c4)cc3)c3ccc(-c4ccc5c(c4)c(-c4ccccc4)c(-c4ccccc4)c4ccccc45)cc3)c21 ; CC1(c2ccccc2)c2ccccc2-c2ccc(N(c3ccc(-c4ccc5ccccc5c4)cc3)c3ccc(-c4ccc5c(c4)c(-c4ccccc4)c(-c4ccccc4)c4ccccc45)cc3)cc21]

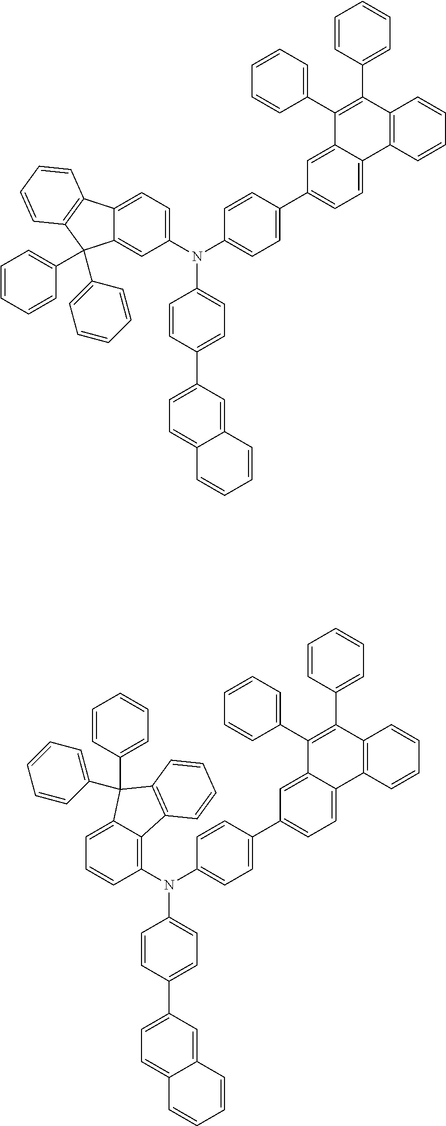 [SMILES: c1ccc(-c2c(-c3ccccc3)c3cc(-c4ccc(N(c5ccc(-c6ccc7ccccc7c6)cc5)c5ccc6c(c5)C(c5ccccc5)(c5ccccc5)c5ccccc5-6)cc4)ccc3c3ccccc23)cc1 ; c1ccc(-c2c(-c3ccccc3)c3cc(-c4ccc(N(c5ccc(-c6ccc7ccccc7c6)cc5)c5cccc6c5-c5ccccc5C6(c5ccccc5)c5ccccc5)cc4)ccc3c3ccccc23)cc1]

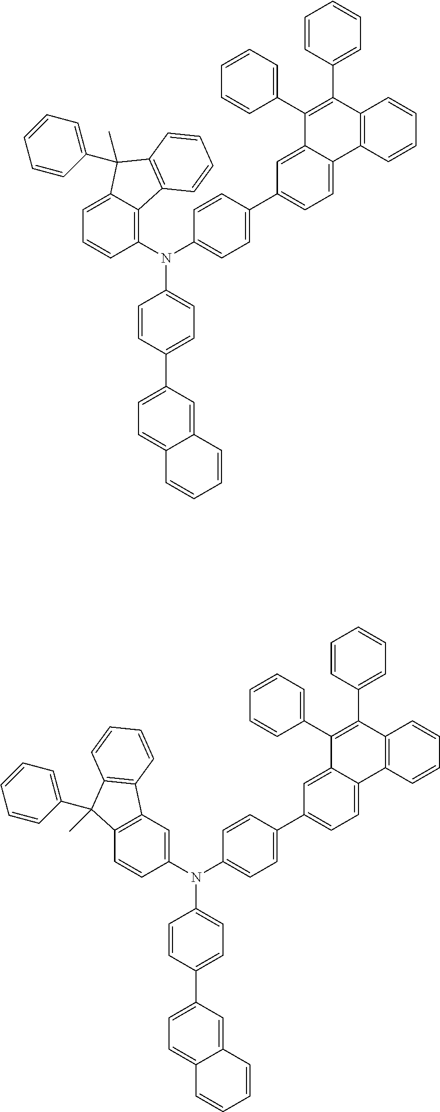 [SMILES: CC1(c2ccccc2)c2ccccc2-c2c(N(c3ccc(-c4ccc5ccccc5c4)cc3)c3ccc(-c4ccc5c(c4)c(-c4ccccc4)c(-c4ccccc4)c4ccccc45)cc3)cccc21 ; CC1(c2ccccc2)c2ccccc2-c2cc(N(c3ccc(-c4ccc5ccccc5c4)cc3)c3ccc(-c4ccc5c(c4)c(-c4ccccc4)c(-c4ccccc4)c4ccccc45)cc3)ccc21]

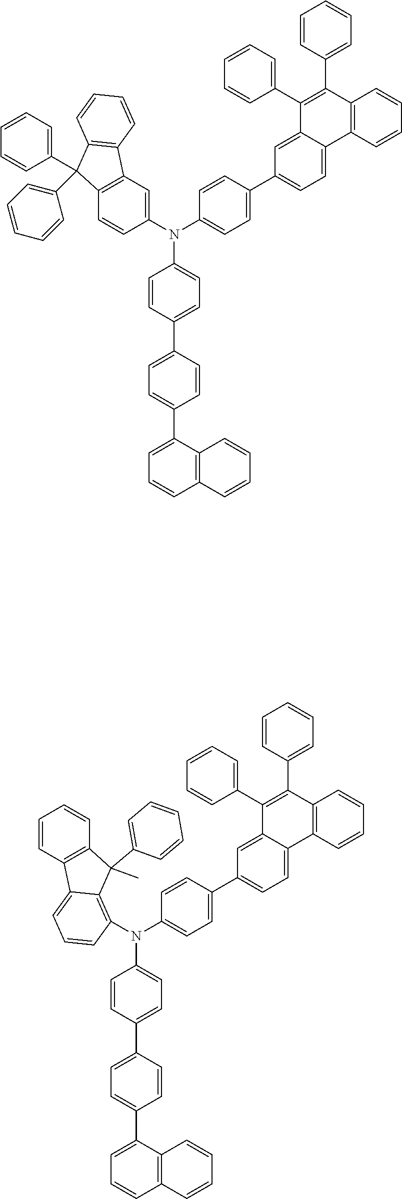 [SMILES: c1ccc(-c2c(-c3ccccc3)c3cc(-c4ccc(N(c5ccc(-c6ccc(-c7cccc8ccccc78)cc6)cc5)c5ccc6c(c5)-c5ccccc5C6(c5ccccc5)c5ccccc5)cc4)ccc3c3ccccc23)cc1 ; CC1(c2ccccc2)c2ccccc2-c2cccc(N(c3ccc(-c4ccc(-c5cccc6ccccc56)cc4)cc3)c3ccc(-c4ccc5c(c4)c(-c4ccccc4)c(-c4ccccc4)c4ccccc45)cc3)c21]

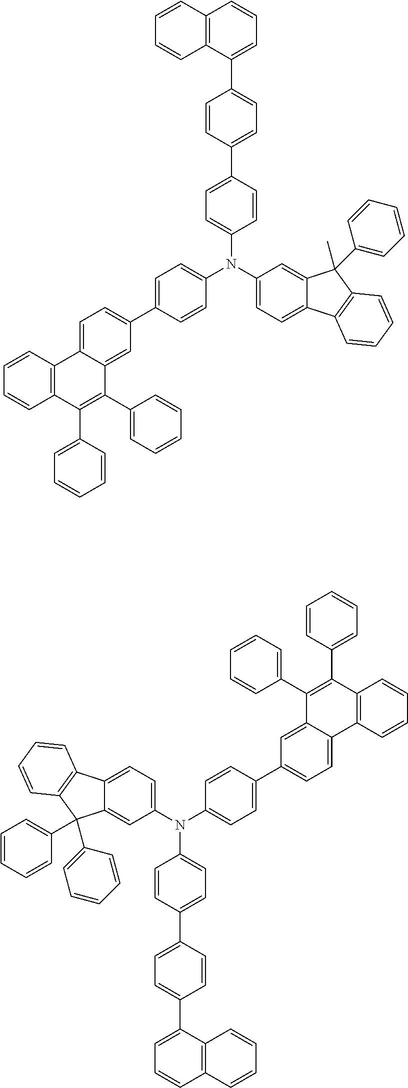 [SMILES: CC1(c2ccccc2)c2ccccc2-c2ccc(N(c3ccc(-c4ccc(-c5cccc6ccccc56)cc4)cc3)c3ccc(-c4ccc5c(c4)c(-c4ccccc4)c(-c4ccccc4)c4ccccc45)cc3)cc21 ; c1ccc(-c2c(-c3ccccc3)c3cc(-c4ccc(N(c5ccc(-c6ccc(-c7cccc8ccccc78)cc6)cc5)c5ccc6c(c5)C(c5ccccc5)(c5ccccc5)c5ccccc5-6)cc4)ccc3c3ccccc23)cc1]

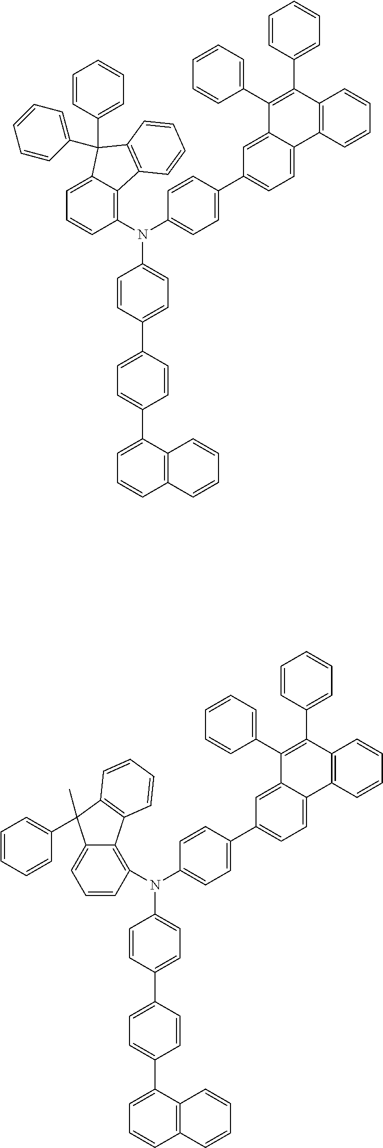 [SMILES: c1ccc(-c2c(-c3ccccc3)c3cc(-c4ccc(N(c5ccc(-c6ccc(-c7cccc8ccccc78)cc6)cc5)c5cccc6c5-c5ccccc5C6(c5ccccc5)c5ccccc5)cc4)ccc3c3ccccc23)cc1 ; CC1(c2ccccc2)c2ccccc2-c2c(N(c3ccc(-c4ccc(-c5cccc6ccccc56)cc4)cc3)c3ccc(-c4ccc5c(c4)c(-c4ccccc4)c(-c4ccccc4)c4ccccc45)cc3)cccc21]

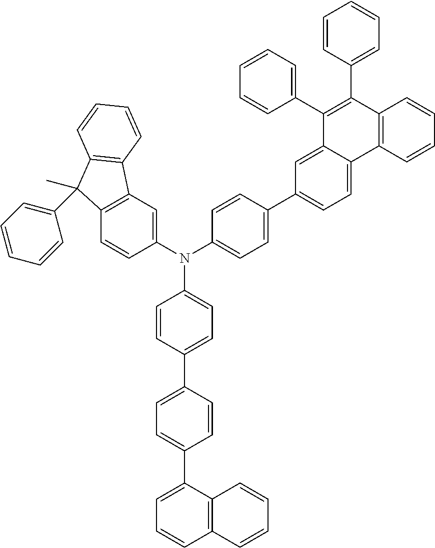 [SMILES: CC1(c2ccccc2)c2ccccc2-c2cc(N(c3ccc(-c4ccc(-c5cccc6ccccc56)cc4)cc3)c3ccc(-c4ccc5c(c4)c(-c4ccccc4)c(-c4ccccc4)c4ccccc45)cc3)ccc21]

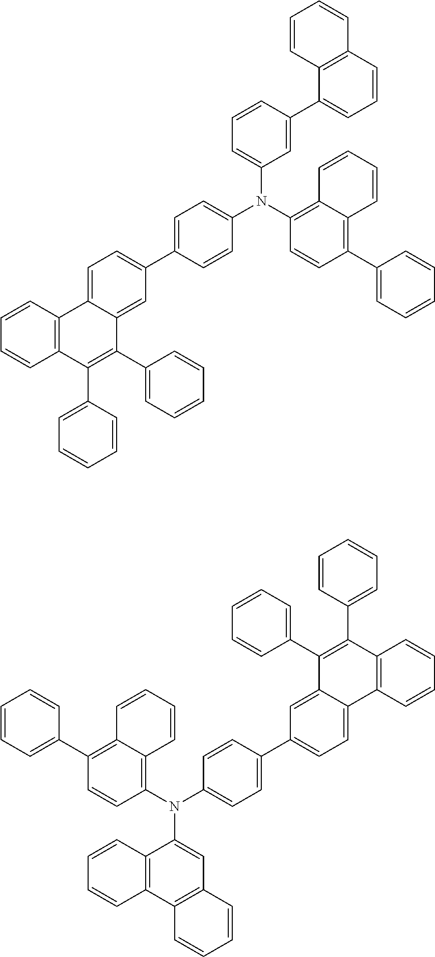 [SMILES: c1ccc(-c2ccc(N(c3ccc(-c4ccc5c(c4)c(-c4ccccc4)c(-c4ccccc4)c4ccccc45)cc3)c3cccc(-c4cccc5ccccc45)c3)c3ccccc23)cc1 ; c1ccc(-c2ccc(N(c3ccc(-c4ccc5c(c4)c(-c4ccccc4)c(-c4ccccc4)c4ccccc45)cc3)c3cc4ccccc4c4ccccc34)c3ccccc23)cc1]

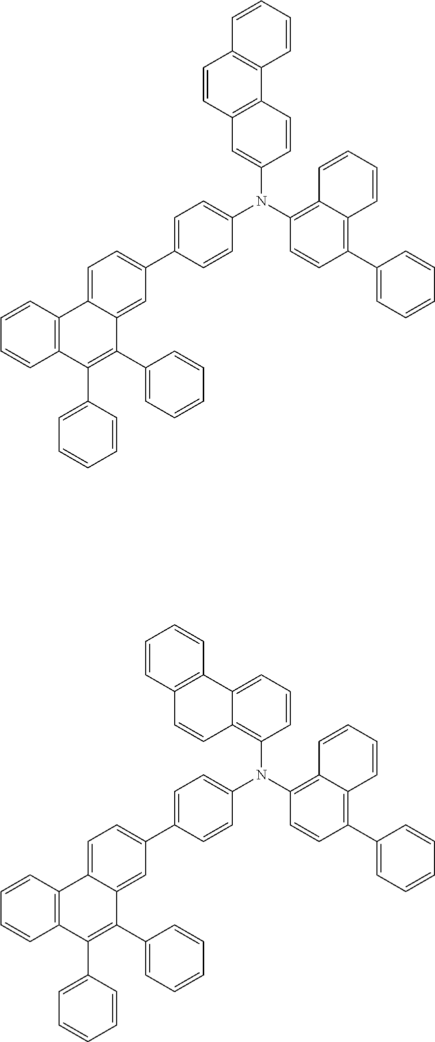 [SMILES: c1ccc(-c2ccc(N(c3ccc(-c4ccc5c(c4)c(-c4ccccc4)c(-c4ccccc4)c4ccccc45)cc3)c3ccc4c(ccc5ccccc54)c3)c3ccccc23)cc1 ; c1ccc(-c2ccc(N(c3ccc(-c4ccc5c(c4)c(-c4ccccc4)c(-c4ccccc4)c4ccccc45)cc3)c3cccc4c3ccc3ccccc34)c3ccccc23)cc1]

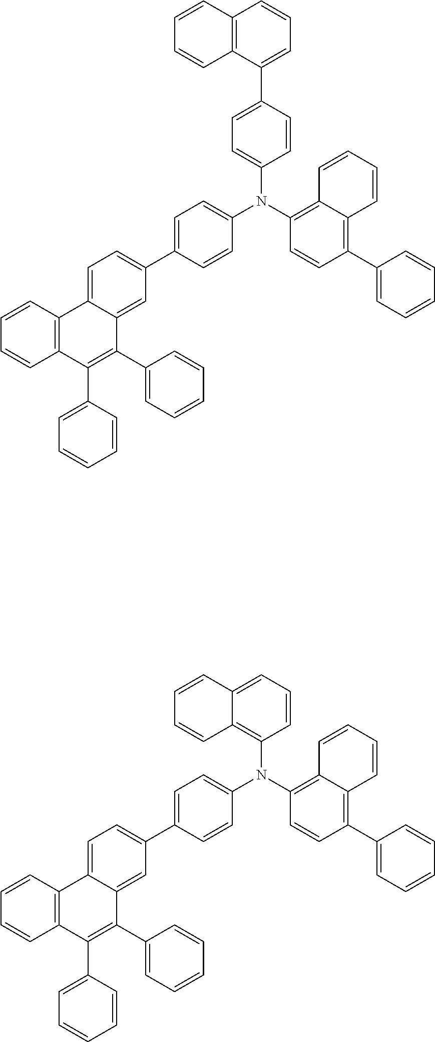 [SMILES: c1ccc(-c2ccc(N(c3ccc(-c4ccc5c(c4)c(-c4ccccc4)c(-c4ccccc4)c4ccccc45)cc3)c3ccc(-c4cccc5ccccc45)cc3)c3ccccc23)cc1 ; c1ccc(-c2ccc(N(c3ccc(-c4ccc5c(c4)c(-c4ccccc4)c(-c4ccccc4)c4ccccc45)cc3)c3cccc4ccccc34)c3ccccc23)cc1]

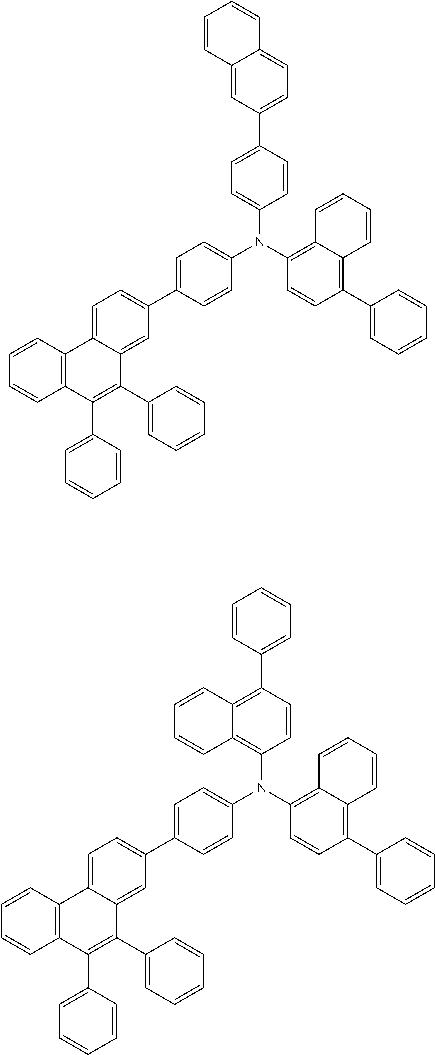 [SMILES: c1ccc(-c2ccc(N(c3ccc(-c4ccc5ccccc5c4)cc3)c3ccc(-c4ccc5c(c4)c(-c4ccccc4)c(-c4ccccc4)c4ccccc45)cc3)c3ccccc23)cc1 ; c1ccc(-c2ccc(N(c3ccc(-c4ccc5c(c4)c(-c4ccccc4)c(-c4ccccc4)c4ccccc45)cc3)c3ccc(-c4ccccc4)c4ccccc34)c3ccccc23)cc1]

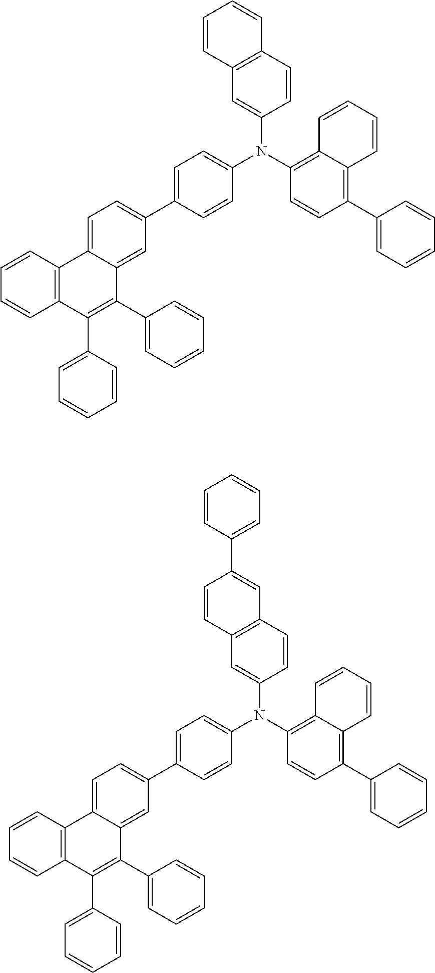 [SMILES: c1ccc(-c2ccc(N(c3ccc(-c4ccc5c(c4)c(-c4ccccc4)c(-c4ccccc4)c4ccccc45)cc3)c3ccc4ccccc4c3)c3ccccc23)cc1 ; c1ccc(-c2ccc3cc(N(c4ccc(-c5ccc6c(c5)c(-c5ccccc5)c(-c5ccccc5)c5ccccc56)cc4)c4ccc(-c5ccccc5)c5ccccc45)ccc3c2)cc1]

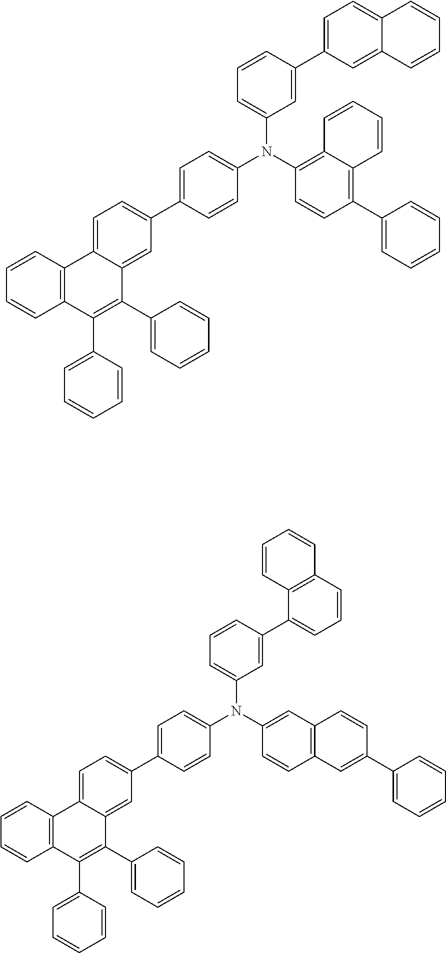 [SMILES: c1ccc(-c2ccc(N(c3ccc(-c4ccc5c(c4)c(-c4ccccc4)c(-c4ccccc4)c4ccccc45)cc3)c3cccc(-c4ccc5ccccc5c4)c3)c3ccccc23)cc1 ; c1ccc(-c2ccc3cc(N(c4ccc(-c5ccc6c(c5)c(-c5ccccc5)c(-c5ccccc5)c5ccccc56)cc4)c4cccc(-c5cccc6ccccc56)c4)ccc3c2)cc1]

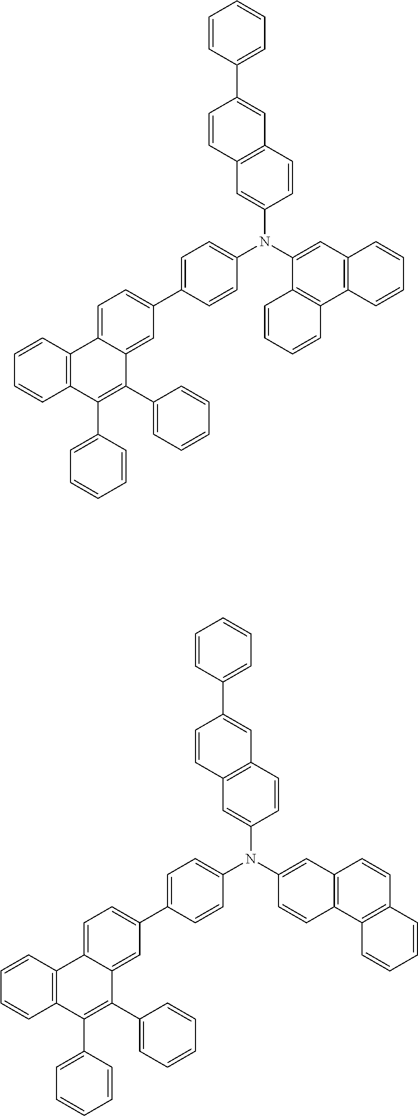 [SMILES: c1ccc(-c2ccc3cc(N(c4ccc(-c5ccc6c(c5)c(-c5ccccc5)c(-c5ccccc5)c5ccccc56)cc4)c4cc5ccccc5c5ccccc45)ccc3c2)cc1 ; c1ccc(-c2ccc3cc(N(c4ccc(-c5ccc6c(c5)c(-c5ccccc5)c(-c5ccccc5)c5ccccc56)cc4)c4ccc5c(ccc6ccccc65)c4)ccc3c2)cc1]

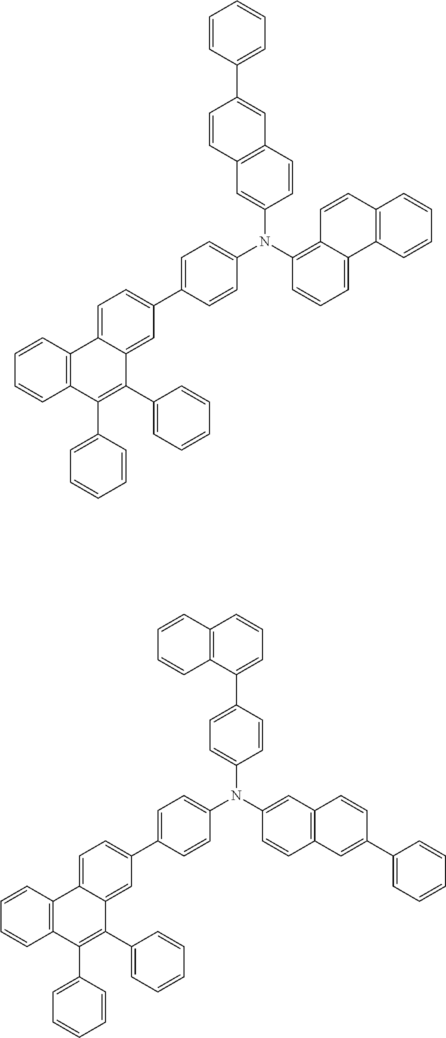 [SMILES: c1ccc(-c2ccc3cc(N(c4ccc(-c5ccc6c(c5)c(-c5ccccc5)c(-c5ccccc5)c5ccccc56)cc4)c4cccc5c4ccc4ccccc45)ccc3c2)cc1 ; c1ccc(-c2ccc3cc(N(c4ccc(-c5ccc6c(c5)c(-c5ccccc5)c(-c5ccccc5)c5ccccc56)cc4)c4ccc(-c5cccc6ccccc56)cc4)ccc3c2)cc1]

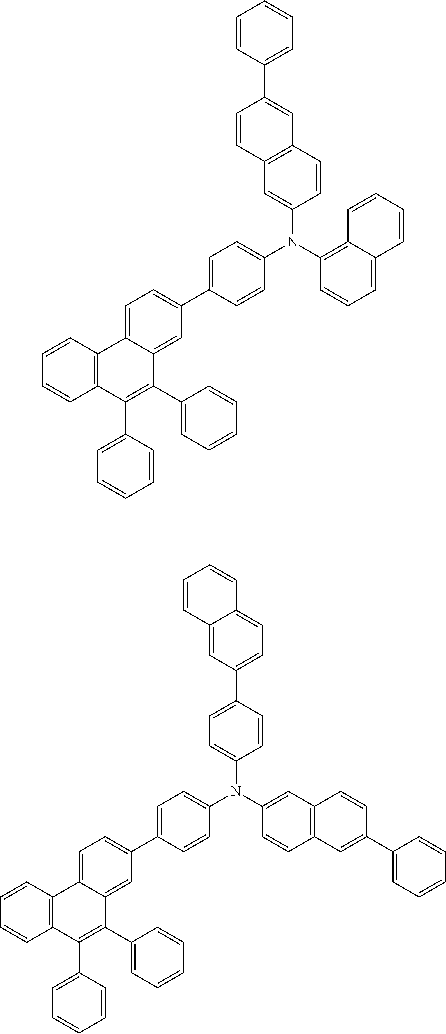 [SMILES: c1ccc(-c2ccc3cc(N(c4ccc(-c5ccc6c(c5)c(-c5ccccc5)c(-c5ccccc5)c5ccccc56)cc4)c4cccc5ccccc45)ccc3c2)cc1 ; c1ccc(-c2ccc3cc(N(c4ccc(-c5ccc6ccccc6c5)cc4)c4ccc(-c5ccc6c(c5)c(-c5ccccc5)c(-c5ccccc5)c5ccccc56)cc4)ccc3c2)cc1]

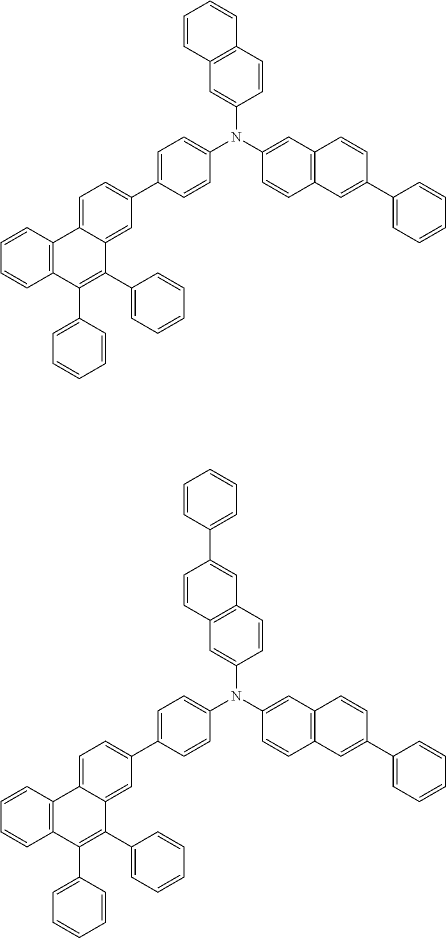 [SMILES: c1ccc(-c2ccc3cc(N(c4ccc(-c5ccc6c(c5)c(-c5ccccc5)c(-c5ccccc5)c5ccccc56)cc4)c4ccc5ccccc5c4)ccc3c2)cc1 ; c1ccc(-c2ccc3cc(N(c4ccc(-c5ccc6c(c5)c(-c5ccccc5)c(-c5ccccc5)c5ccccc56)cc4)c4ccc5cc(-c6ccccc6)ccc5c4)ccc3c2)cc1]

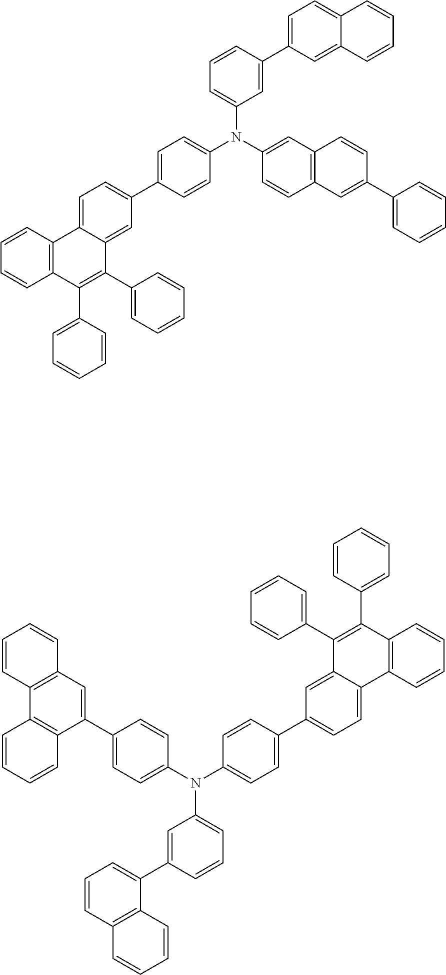 [SMILES: c1ccc(-c2ccc3cc(N(c4ccc(-c5ccc6c(c5)c(-c5ccccc5)c(-c5ccccc5)c5ccccc56)cc4)c4cccc(-c5ccc6ccccc6c5)c4)ccc3c2)cc1 ; c1ccc(-c2c(-c3ccccc3)c3cc(-c4ccc(N(c5ccc(-c6cc7ccccc7c7ccccc67)cc5)c5cccc(-c6cccc7ccccc67)c5)cc4)ccc3c3ccccc23)cc1]

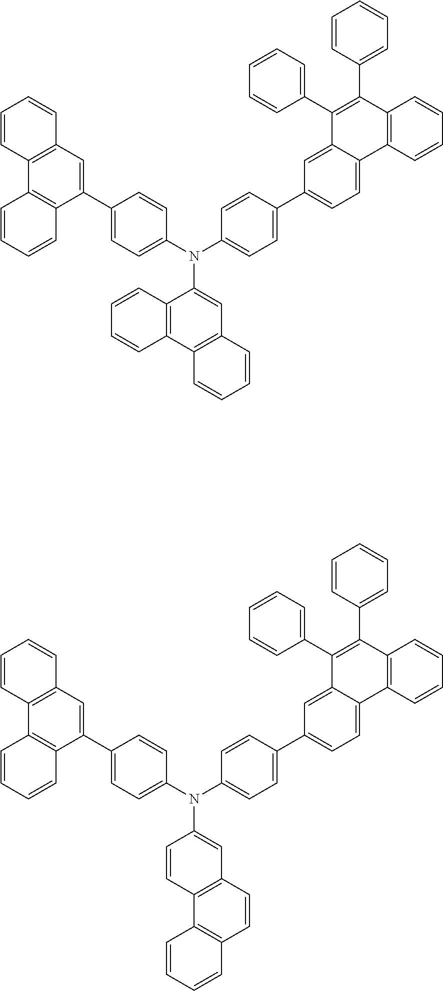 [SMILES: c1ccc(-c2c(-c3ccccc3)c3cc(-c4ccc(N(c5ccc(-c6cc7ccccc7c7ccccc67)cc5)c5cc6ccccc6c6ccccc56)cc4)ccc3c3ccccc23)cc1 ; c1ccc(-c2c(-c3ccccc3)c3cc(-c4ccc(N(c5ccc(-c6cc7ccccc7c7ccccc67)cc5)c5ccc6c(ccc7ccccc76)c5)cc4)ccc3c3ccccc23)cc1]

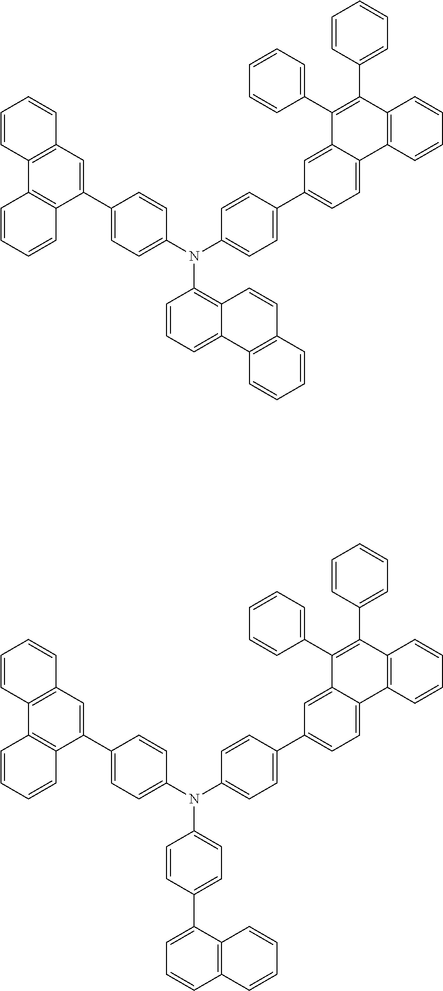 [SMILES: c1ccc(-c2c(-c3ccccc3)c3cc(-c4ccc(N(c5ccc(-c6cc7ccccc7c7ccccc67)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc(-c6cc7ccccc7c7ccccc67)cc5)cc4)ccc3c3ccccc23)cc1]

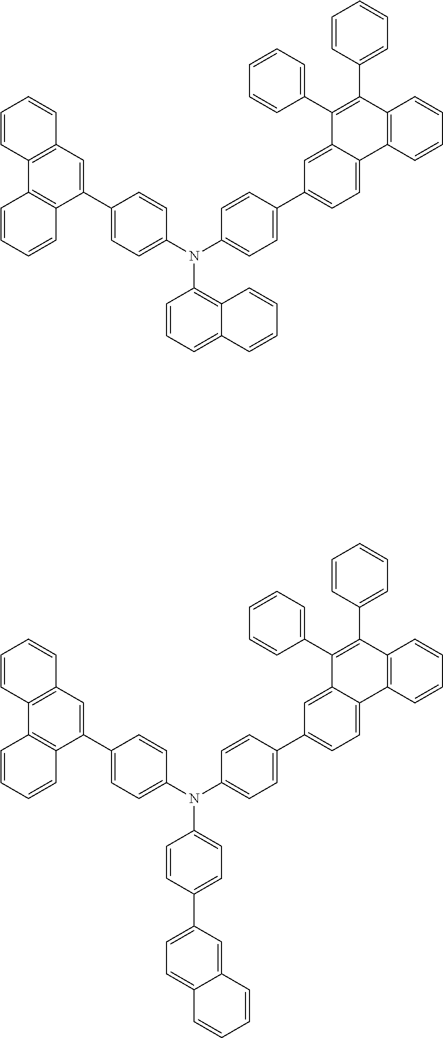 [SMILES: c1ccc(-c2c(-c3ccccc3)c3cc(-c4ccc(N(c5ccc(-c6cc7ccccc7c7ccccc67)cc5)c5cccc6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6cc7ccccc7c7ccccc67)cc5)cc4)ccc3c3ccccc23)cc1]

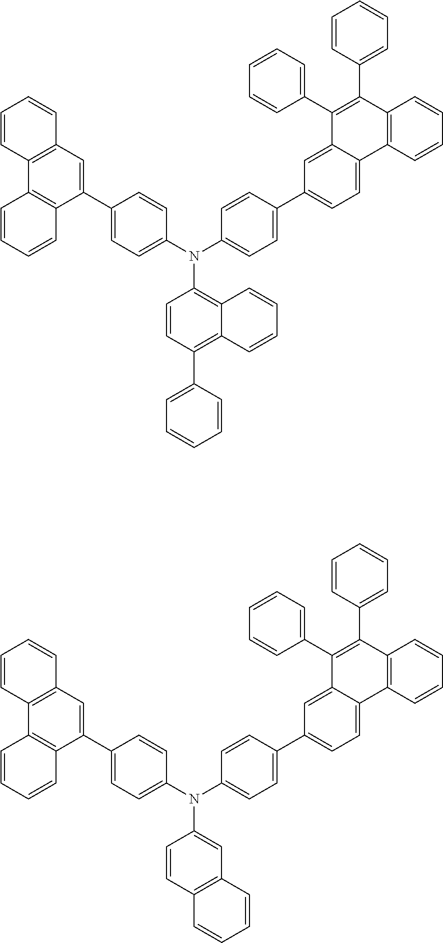 [SMILES: c1ccc(-c2ccc(N(c3ccc(-c4ccc5c(c4)c(-c4ccccc4)c(-c4ccccc4)c4ccccc45)cc3)c3ccc(-c4cc5ccccc5c5ccccc45)cc3)c3ccccc23)cc1 ; c1ccc(-c2c(-c3ccccc3)c3cc(-c4ccc(N(c5ccc(-c6cc7ccccc7c7ccccc67)cc5)c5ccc6ccccc6c5)cc4)ccc3c3ccccc23)cc1]

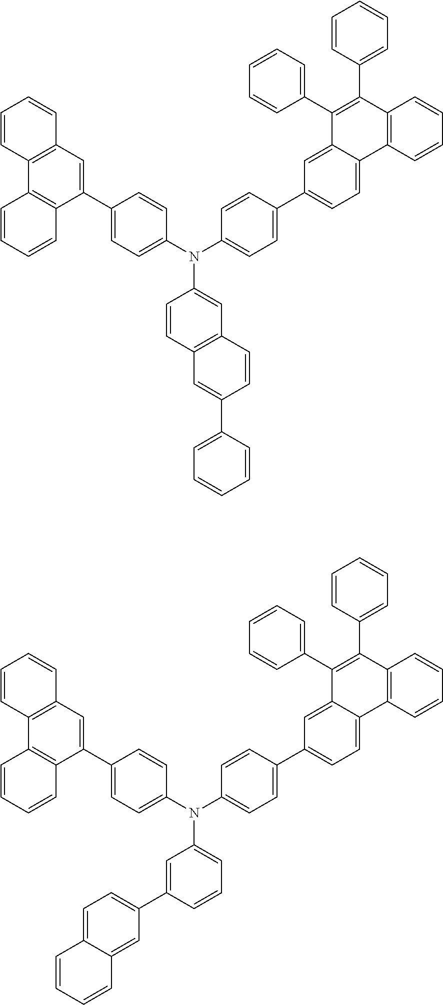 [SMILES: c1ccc(-c2ccc3cc(N(c4ccc(-c5ccc6c(c5)c(-c5ccccc5)c(-c5ccccc5)c5ccccc56)cc4)c4ccc(-c5cc6ccccc6c6ccccc56)cc4)ccc3c2)cc1 ; c1ccc(-c2c(-c3ccccc3)c3cc(-c4ccc(N(c5ccc(-c6cc7ccccc7c7ccccc67)cc5)c5cccc(-c6ccc7ccccc7c6)c5)cc4)ccc3c3ccccc23)cc1]

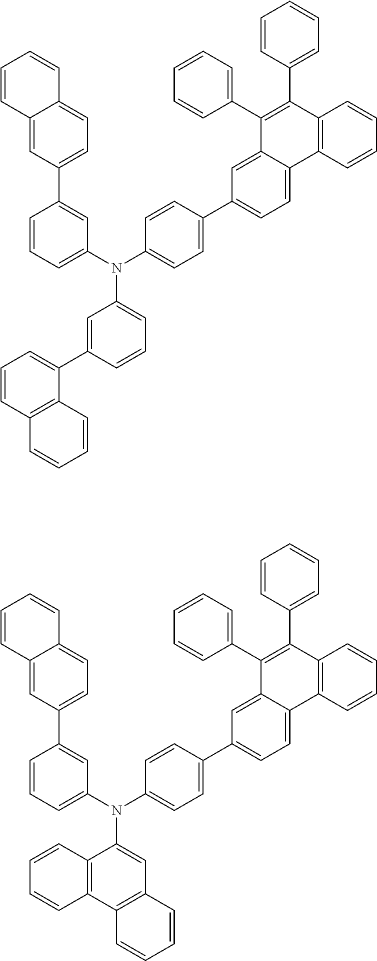 [SMILES: c1ccc(-c2c(-c3ccccc3)c3cc(-c4ccc(N(c5cccc(-c6ccc7ccccc7c6)c5)c5cccc(-c6cccc7ccccc67)c5)cc4)ccc3c3ccccc23)cc1 ; c1ccc(-c2c(-c3ccccc3)c3cc(-c4ccc(N(c5cccc(-c6ccc7ccccc7c6)c5)c5cc6ccccc6c6ccccc56)cc4)ccc3c3ccccc23)cc1]

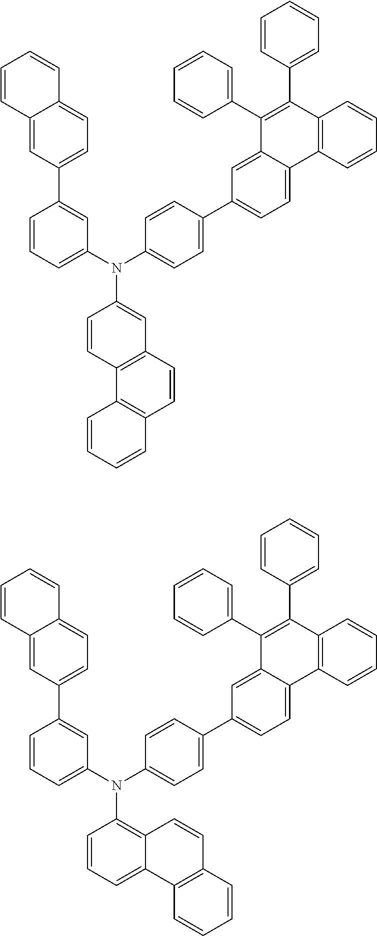 [SMILES: c1ccc(-c2c(-c3ccccc3)c3cc(-c4ccc(N(c5cccc(-c6ccc7ccccc7c6)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6ccc7ccccc7c6)c5)c5cccc6c5ccc5ccccc56)cc4)ccc3c3ccccc23)cc1]

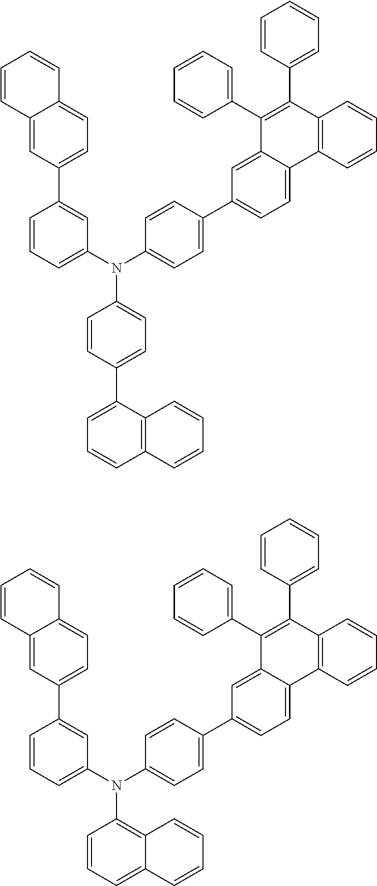 [SMILES: c1ccc(-c2c(-c3ccccc3)c3cc(-c4ccc(N(c5ccc(-c6cccc7ccccc67)cc5)c5cccc(-c6ccc7ccccc7c6)c5)cc4)ccc3c3ccccc23)cc1 ; c1ccc(-c2c(-c3ccccc3)c3cc(-c4ccc(N(c5cccc(-c6ccc7ccccc7c6)c5)c5cccc6ccccc56)cc4)ccc3c3ccccc23)cc1]

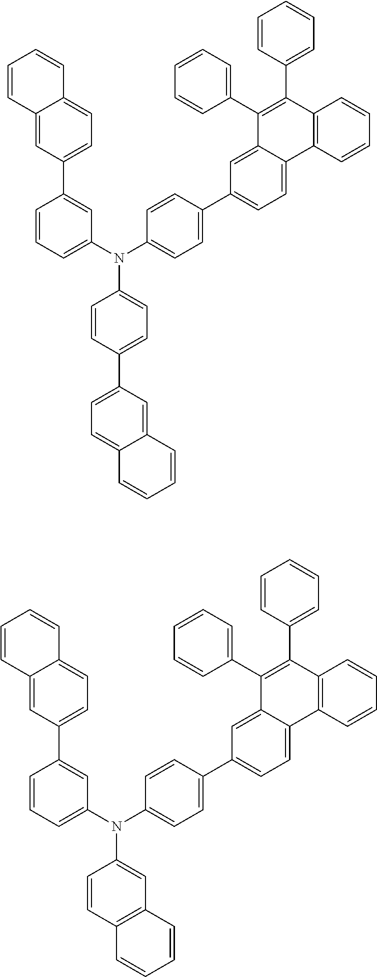 [SMILES: c1ccc(-c2c(-c3ccccc3)c3cc(-c4ccc(N(c5ccc(-c6ccc7ccccc7c6)cc5)c5cccc(-c6ccc7ccccc7c6)c5)cc4)ccc3c3ccccc23)cc1 ; c1ccc(-c2c(-c3ccccc3)c3cc(-c4ccc(N(c5cccc(-c6ccc7ccccc7c6)c5)c5ccc6ccccc6c5)cc4)ccc3c3ccccc23)cc1]

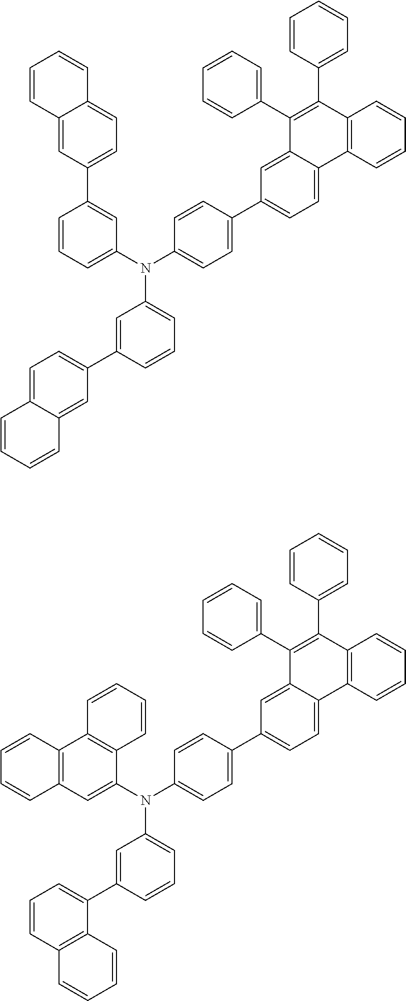 [SMILES: c1ccc(-c2c(-c3ccccc3)c3cc(-c4ccc(N(c5cccc(-c6ccc7ccccc7c6)c5)c5cccc(-c6ccc7ccccc7c6)c5)cc4)ccc3c3ccccc23)cc1 ; c1ccc(-c2c(-c3ccccc3)c3cc(-c4ccc(N(c5cccc(-c6cccc7ccccc67)c5)c5cc6ccccc6c6ccccc56)cc4)ccc3c3ccccc23)cc1]

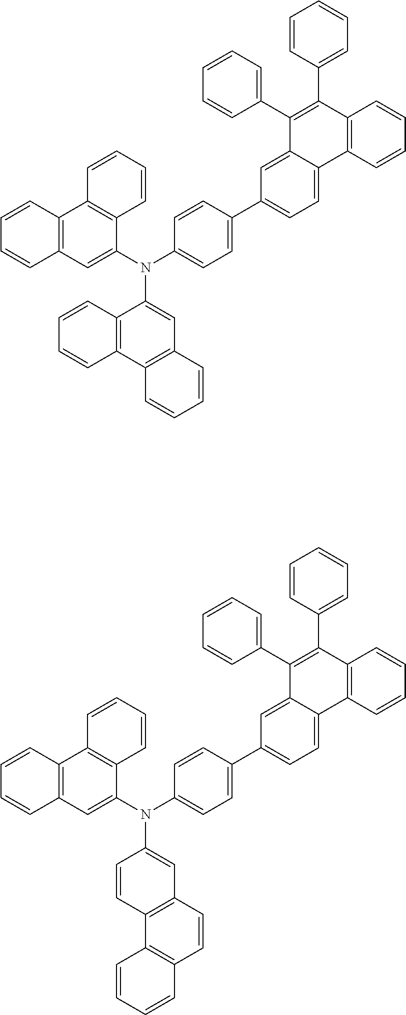 [SMILES: c1ccc(-c2c(-c3ccccc3)c3cc(-c4ccc(N(c5cc6ccccc6c6ccccc56)c5cc6ccccc6c6ccccc56)cc4)ccc3c3ccccc23)cc1 ; c1ccc(-c2c(-c3ccccc3)c3cc(-c4ccc(N(c5ccc6c(ccc7ccccc76)c5)c5cc6ccccc6c6ccccc56)cc4)ccc3c3ccccc23)cc1]

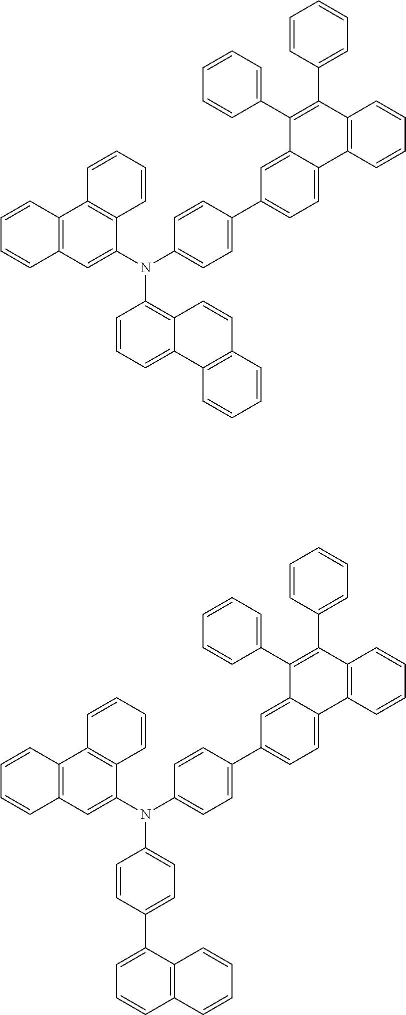 [SMILES: c1ccc(-c2c(-c3ccccc3)c3cc(-c4ccc(N(c5cccc6c5ccc5ccccc56)c5cc6ccccc6c6ccccc56)cc4)ccc3c3ccccc23)cc1 ; c1ccc(-c2c(-c3ccccc3)c3cc(-c4ccc(N(c5ccc(-c6cccc7ccccc67)cc5)c5cc6ccccc6c6ccccc56)cc4)ccc3c3ccccc23)cc1]

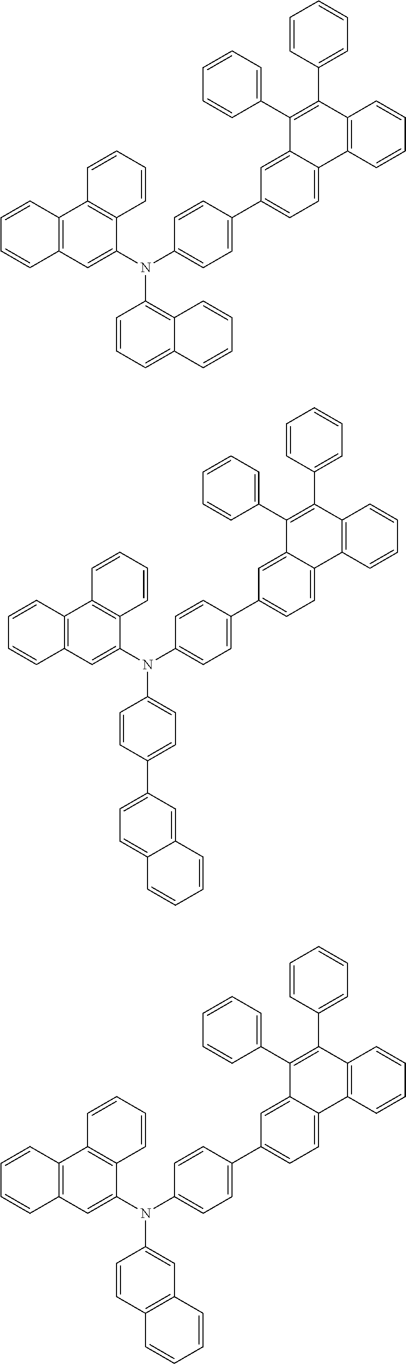 [SMILES: c1ccc(-c2c(-c3ccccc3)c3cc(-c4ccc(N(c5cccc6ccccc56)c5cc6ccccc6c6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5cc6ccccc6c6ccccc56)cc4)ccc3c3ccccc23)cc1 ; c1ccc(-c2c(-c3ccccc3)c3cc(-c4ccc(N(c5ccc6ccccc6c5)c5cc6ccccc6c6ccccc56)cc4)ccc3c3ccccc23)cc1]

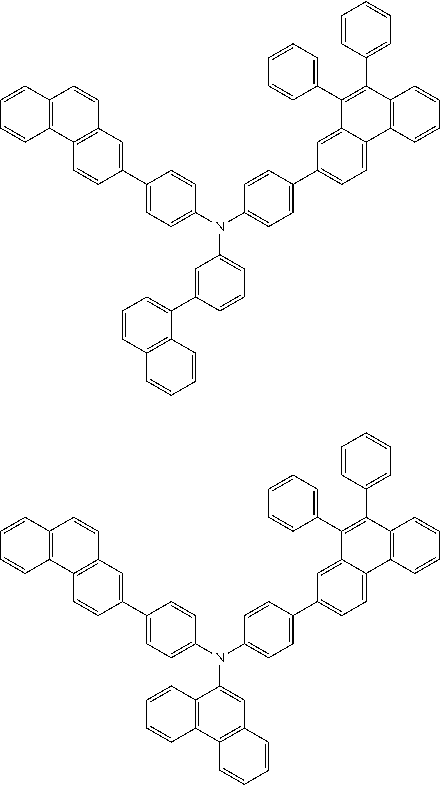 [SMILES: c1ccc(-c2c(-c3ccccc3)c3cc(-c4ccc(N(c5ccc(-c6ccc7c(ccc8ccccc87)c6)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6ccc7c(ccc8ccccc87)c6)cc5)c5cc6ccccc6c6ccccc56)cc4)ccc3c3ccccc23)cc1]

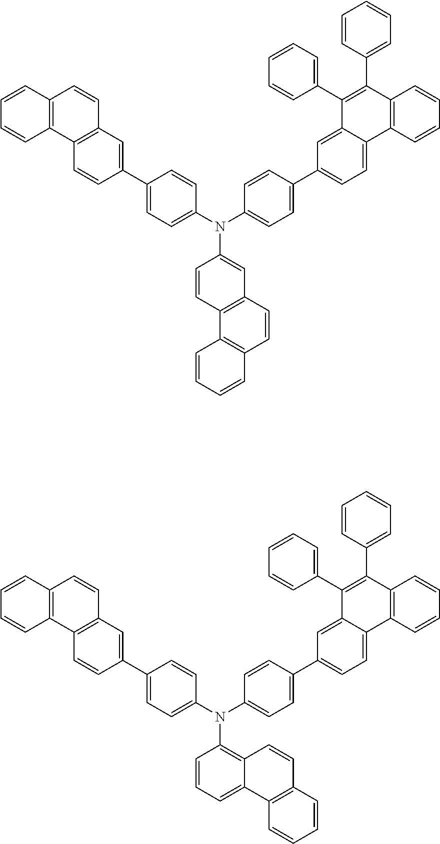 [SMILES: c1ccc(-c2c(-c3ccccc3)c3cc(-c4ccc(N(c5ccc(-c6ccc7c(ccc8ccccc87)c6)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6ccc7c(ccc8ccccc87)c6)cc5)c5cccc6c5ccc5ccccc56)cc4)ccc3c3ccccc23)cc1]

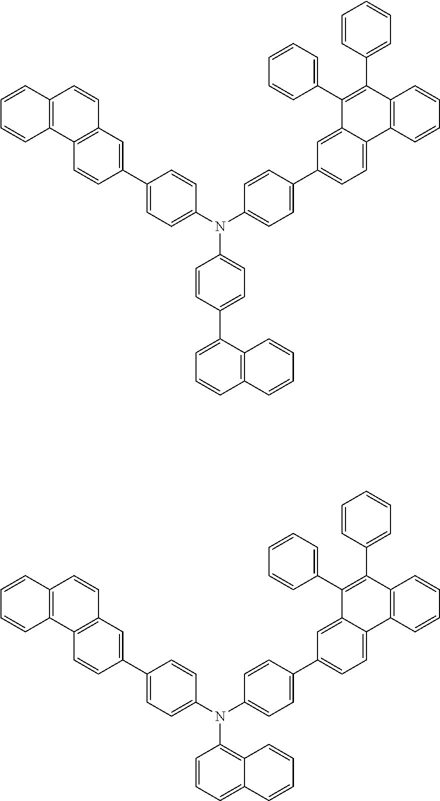 [SMILES: c1ccc(-c2c(-c3ccccc3)c3cc(-c4ccc(N(c5ccc(-c6ccc7c(ccc8ccccc87)c6)cc5)c5ccc(-c6cccc7ccccc67)cc5)cc4)ccc3c3ccccc23)cc1 ; c1ccc(-c2c(-c3ccccc3)c3cc(-c4ccc(N(c5ccc(-c6ccc7c(ccc8ccccc87)c6)cc5)c5cccc6ccccc56)cc4)ccc3c3ccccc23)cc1]

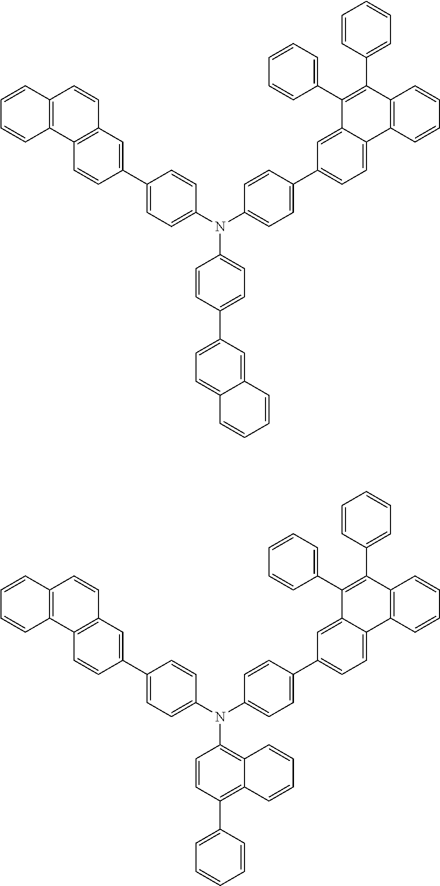 [SMILES: c1ccc(-c2c(-c3ccccc3)c3cc(-c4ccc(N(c5ccc(-c6ccc7ccccc7c6)cc5)c5ccc(-c6ccc7c(ccc8ccccc87)c6)cc5)cc4)ccc3c3ccccc23)cc1 ; c1ccc(-c2ccc(N(c3ccc(-c4ccc5c(ccc6ccccc65)c4)cc3)c3ccc(-c4ccc5c(c4)c(-c4ccccc4)c(-c4ccccc4)c4ccccc45)cc3)c3ccccc23)cc1]

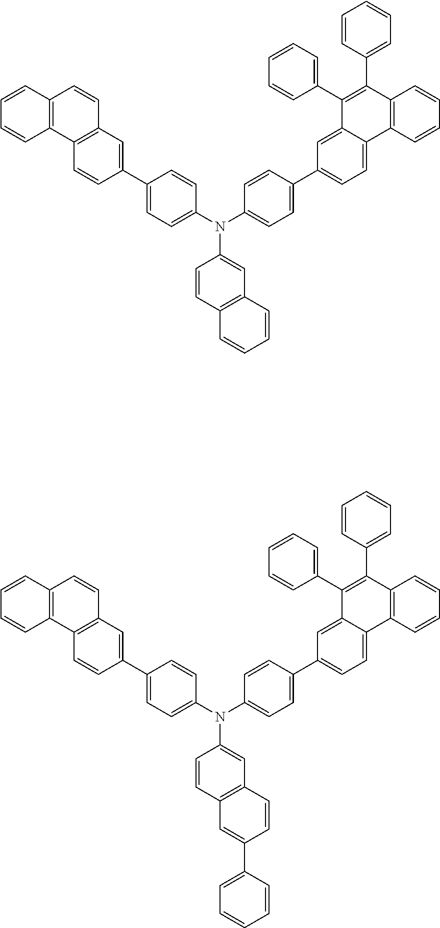 [SMILES: c1ccc(-c2c(-c3ccccc3)c3cc(-c4ccc(N(c5ccc(-c6ccc7c(ccc8ccccc87)c6)cc5)c5ccc6ccccc6c5)cc4)ccc3c3ccccc23)cc1 ; c1ccc(-c2ccc3cc(N(c4ccc(-c5ccc6c(ccc7ccccc76)c5)cc4)c4ccc(-c5ccc6c(c5)c(-c5ccccc5)c(-c5ccccc5)c5ccccc56)cc4)ccc3c2)cc1]

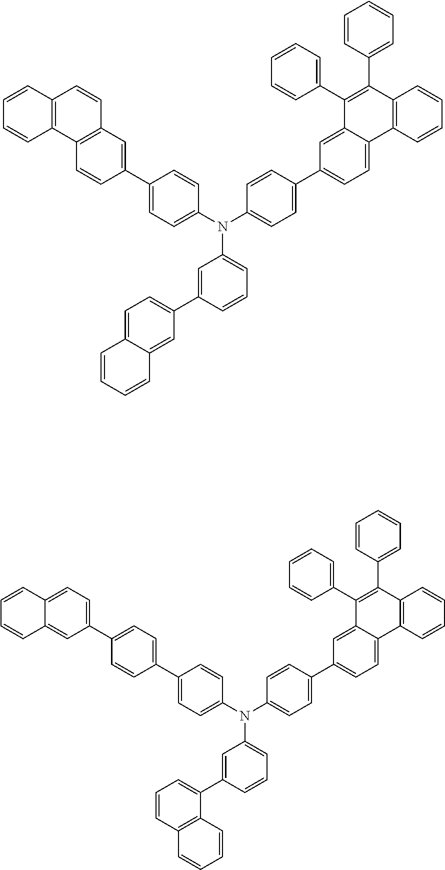 [SMILES: c1ccc(-c2c(-c3ccccc3)c3cc(-c4ccc(N(c5ccc(-c6ccc7c(ccc8ccccc87)c6)cc5)c5cccc(-c6ccc7ccccc7c6)c5)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5cccc(-c6cccc7ccccc67)c5)cc4)ccc3c3ccccc23)cc1]

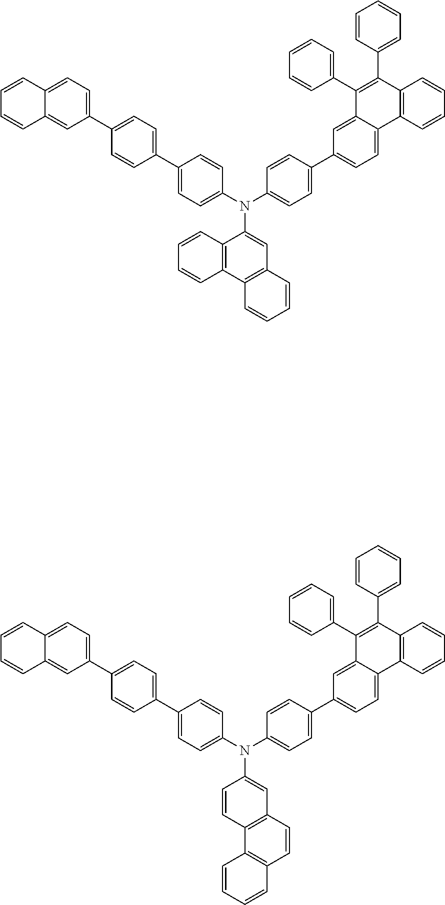 [SMILES: c1ccc(-c2c(-c3ccccc3)c3cc(-c4ccc(N(c5ccc(-c6ccc(-c7ccc8ccccc8c7)cc6)cc5)c5cc6ccccc6c6ccccc56)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5ccc6c(ccc7ccccc76)c5)cc4)ccc3c3ccccc23)cc1]

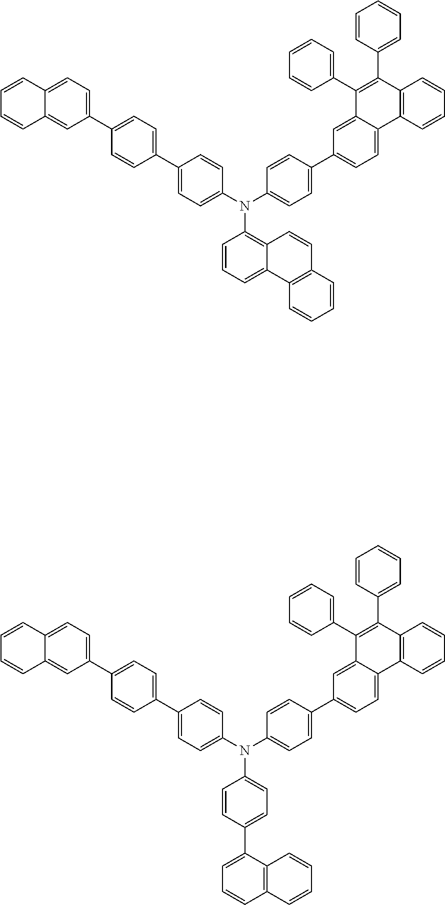 [SMILES: c1ccc(-c2c(-c3ccccc3)c3cc(-c4ccc(N(c5ccc(-c6ccc(-c7ccc8ccccc8c7)cc6)cc5)c5cccc6c5ccc5ccccc56)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5ccc(-c6cccc7ccccc67)cc5)cc4)ccc3c3ccccc23)cc1]

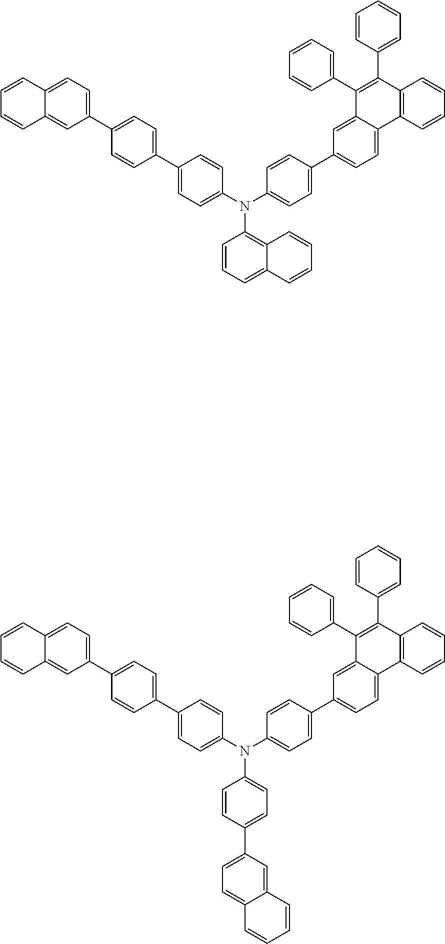 [SMILES: c1ccc(-c2c(-c3ccccc3)c3cc(-c4ccc(N(c5ccc(-c6ccc(-c7ccc8ccccc8c7)cc6)cc5)c5cccc6ccccc56)cc4)ccc3c3ccccc23)cc1 ; c1ccc(-c2c(-c3ccccc3)c3cc(-c4ccc(N(c5ccc(-c6ccc(-c7ccc8ccccc8c7)cc6)cc5)c5ccc(-c6ccc7ccccc7c6)cc5)cc4)ccc3c3ccccc23)cc1]

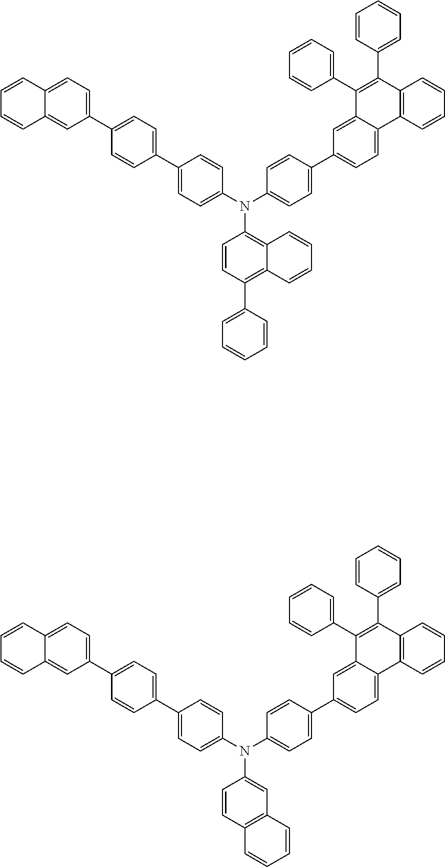 [SMILES: c1ccc(-c2ccc(N(c3ccc(-c4ccc(-c5ccc6ccccc6c5)cc4)cc3)c3ccc(-c4ccc5c(c4)c(-c4ccccc4)c(-c4ccccc4)c4ccccc45)cc3)c3ccccc23)cc1 ; c1ccc(-c2c(-c3ccccc3)c3cc(-c4ccc(N(c5ccc(-c6ccc(-c7ccc8ccccc8c7)cc6)cc5)c5ccc6ccccc6c5)cc4)ccc3c3ccccc23)cc1]

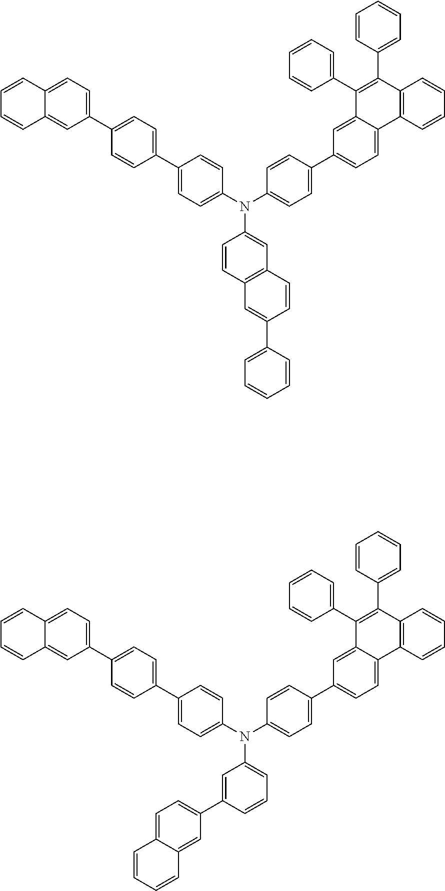 [SMILES: c1ccc(-c2ccc3cc(N(c4ccc(-c5ccc(-c6ccc7ccccc7c6)cc5)cc4)c4ccc(-c5ccc6c(c5)c(-c5ccccc5)c(-c5ccccc5)c5ccccc56)cc4)ccc3c2)cc1 ; c1ccc(-c2c(-c3ccccc3)c3cc(-c4ccc(N(c5ccc(-c6ccc(-c7ccc8ccccc8c7)cc6)cc5)c5cccc(-c6ccc7ccccc7c6)c5)cc4)ccc3c3ccccc23)cc1]

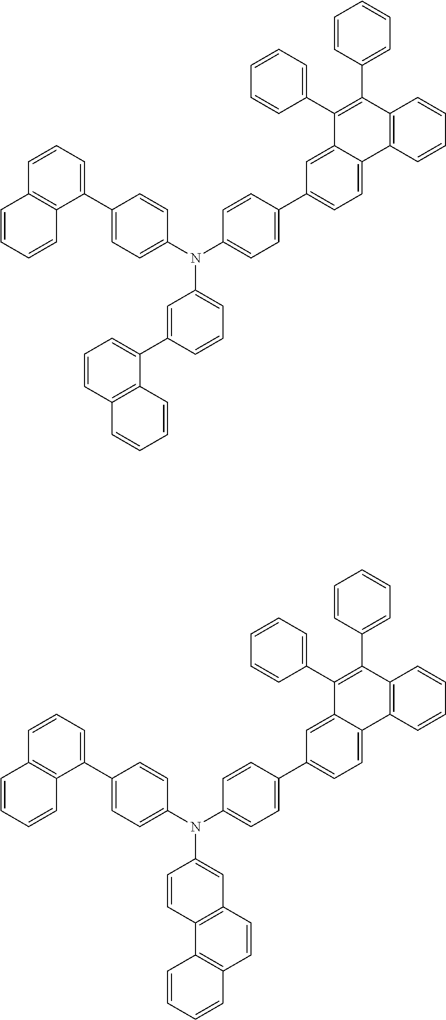 [SMILES: c1ccc(-c2c(-c3ccccc3)c3cc(-c4ccc(N(c5ccc(-c6cccc7ccccc67)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6cccc7ccccc67)cc5)c5ccc6c(ccc7ccccc76)c5)cc4)ccc3c3ccccc23)cc1]

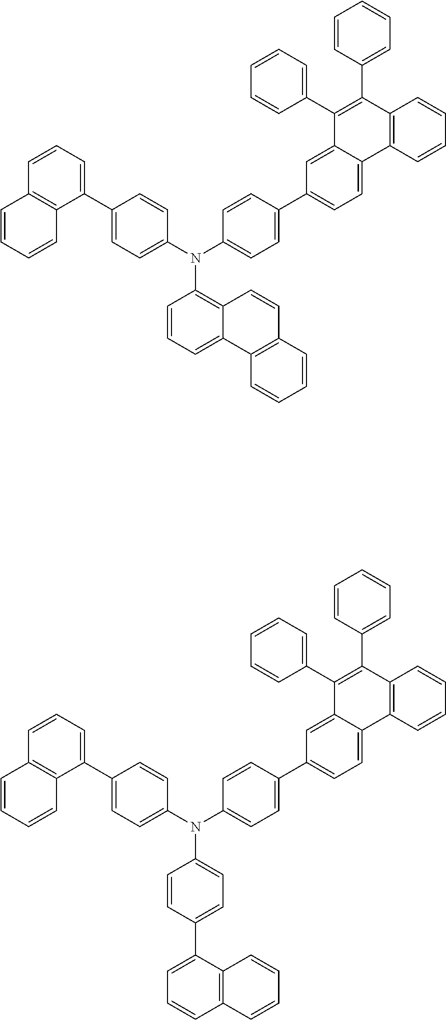 [SMILES: c1ccc(-c2c(-c3ccccc3)c3cc(-c4ccc(N(c5ccc(-c6cccc7ccccc67)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc(-c6cccc7ccccc67)cc5)cc4)ccc3c3ccccc23)cc1]

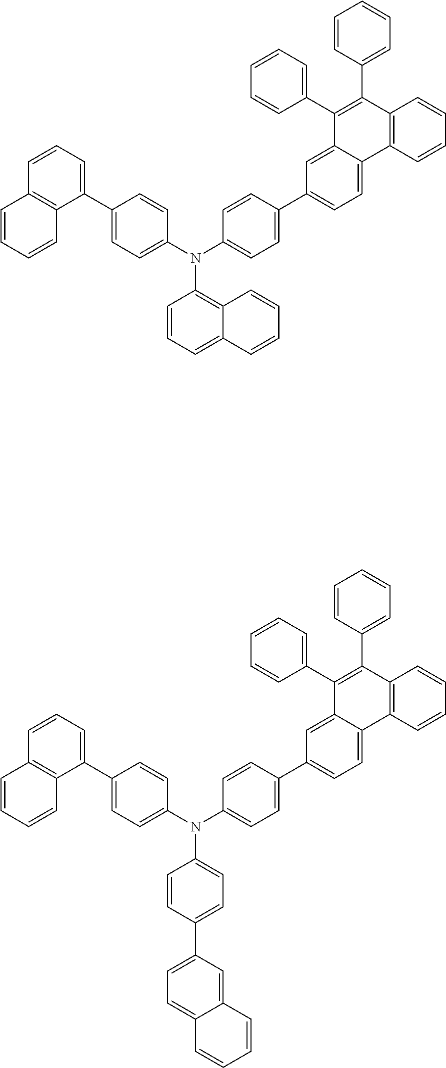 [SMILES: c1ccc(-c2c(-c3ccccc3)c3cc(-c4ccc(N(c5ccc(-c6cccc7ccccc67)cc5)c5cccc6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6cccc7ccccc67)cc5)cc4)ccc3c3ccccc23)cc1]

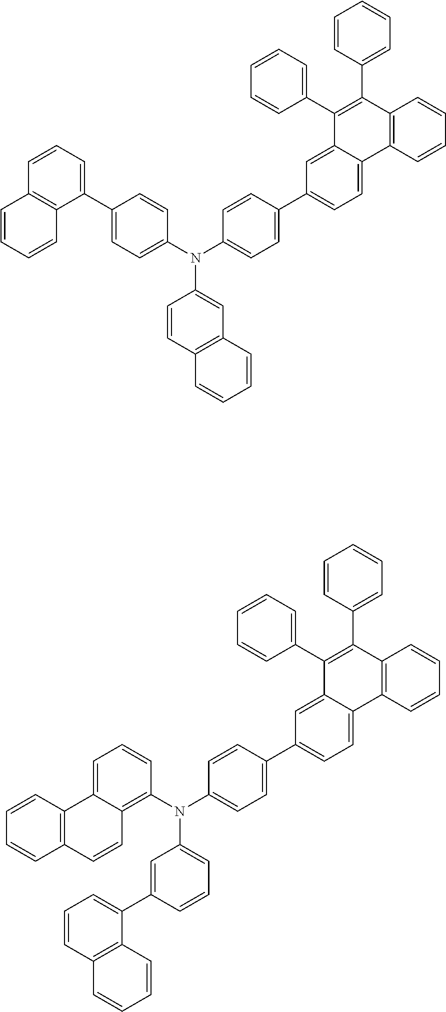 [SMILES: c1ccc(-c2c(-c3ccccc3)c3cc(-c4ccc(N(c5ccc(-c6cccc7ccccc67)cc5)c5ccc6ccccc6c5)cc4)ccc3c3ccccc23)cc1 ; c1ccc(-c2c(-c3ccccc3)c3cc(-c4ccc(N(c5cccc(-c6cccc7ccccc67)c5)c5cccc6c5ccc5ccccc56)cc4)ccc3c3ccccc23)cc1]

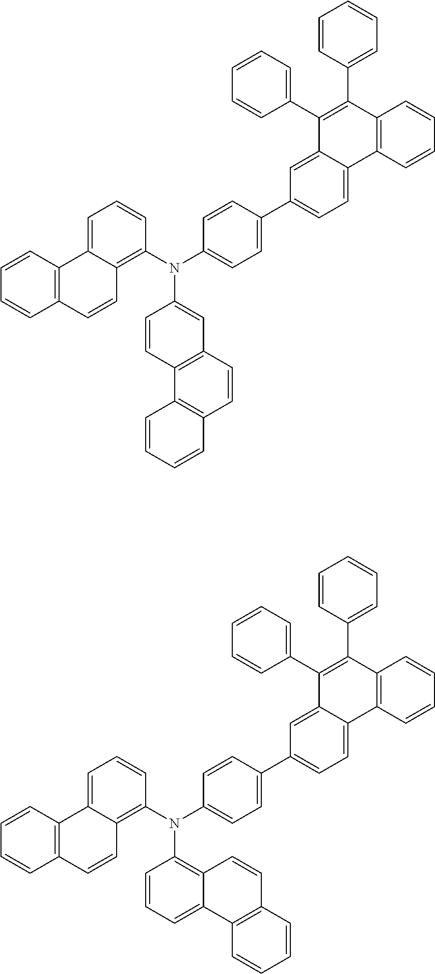 [SMILES: c1ccc(-c2c(-c3ccccc3)c3cc(-c4ccc(N(c5ccc6c(ccc7ccccc76)c5)c5cccc6c5ccc5ccccc56)cc4)ccc3c3ccccc23)cc1 ; c1ccc(-c2c(-c3ccccc3)c3cc(-c4ccc(N(c5cccc6c5ccc5ccccc56)c5cccc6c5ccc5ccccc56)cc4)ccc3c3ccccc23)cc1]

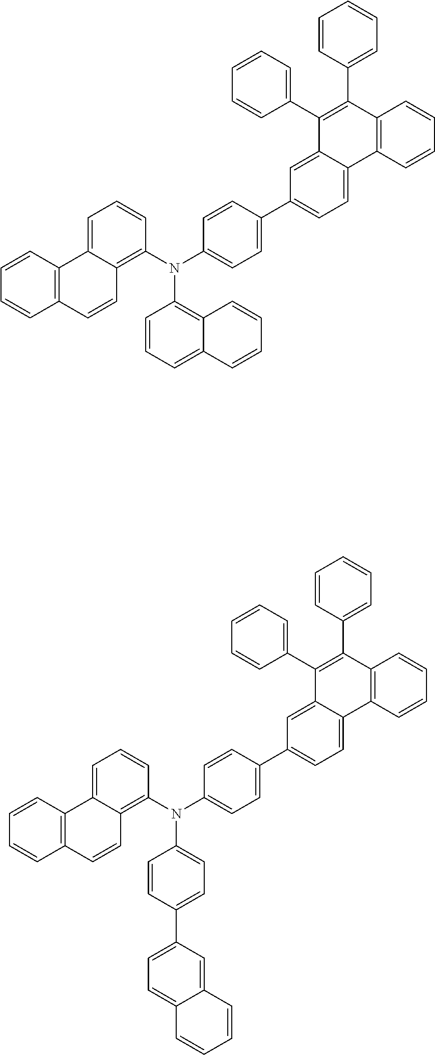 [SMILES: c1ccc(-c2c(-c3ccccc3)c3cc(-c4ccc(N(c5cccc6ccccc56)c5cccc6c5ccc5ccccc56)cc4)ccc3c3ccccc23)cc1 ; c1ccc(-c2c(-c3ccccc3)c3cc(-c4ccc(N(c5ccc(-c6ccc7ccccc7c6)cc5)c5cccc6c5ccc5ccccc56)cc4)ccc3c3ccccc23)cc1]

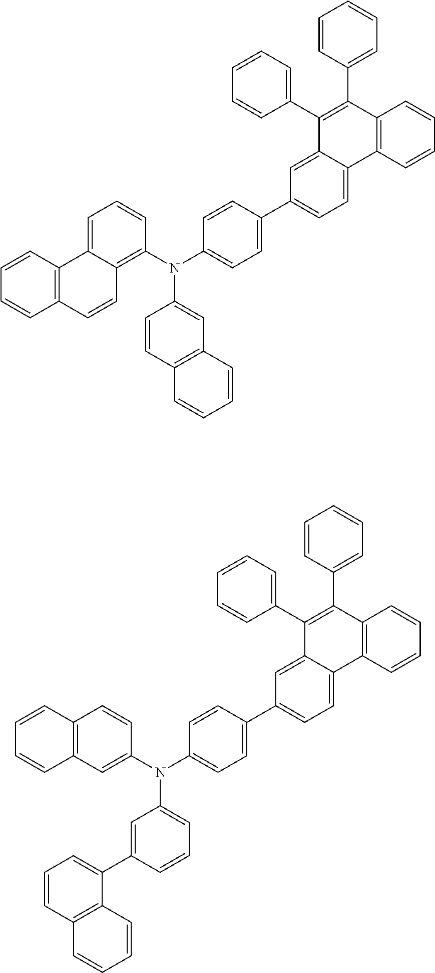 [SMILES: c1ccc(-c2c(-c3ccccc3)c3cc(-c4ccc(N(c5ccc6ccccc6c5)c5cccc6c5ccc5ccccc56)cc4)ccc3c3ccccc23)cc1 ; c1ccc(-c2c(-c3ccccc3)c3cc(-c4ccc(N(c5cccc(-c6cccc7ccccc67)c5)c5ccc6ccccc6c5)cc4)ccc3c3ccccc23)cc1]

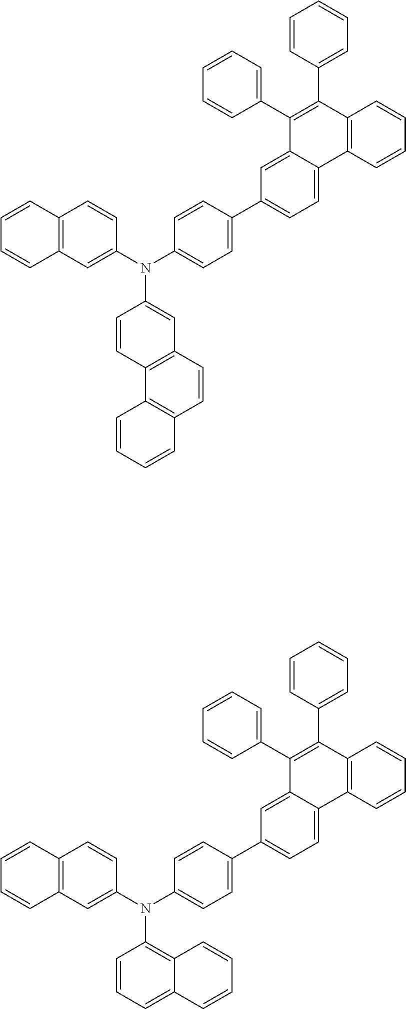 [SMILES: c1ccc(-c2c(-c3ccccc3)c3cc(-c4ccc(N(c5ccc6ccccc6c5)c5ccc6c(ccc7ccccc76)c5)cc4)ccc3c3ccccc23)cc1 ; c1ccc(-c2c(-c3ccccc3)c3cc(-c4ccc(N(c5ccc6ccccc6c5)c5cccc6ccccc56)cc4)ccc3c3ccccc23)cc1]

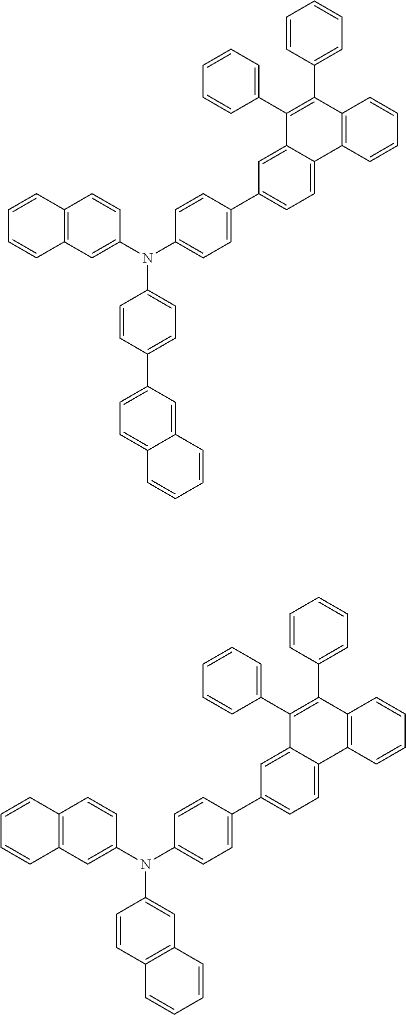 [SMILES: c1ccc(-c2c(-c3ccccc3)c3cc(-c4ccc(N(c5ccc(-c6ccc7ccccc7c6)cc5)c5ccc6ccccc6c5)cc4)ccc3c3ccccc23)cc1 ; c1ccc(-c2c(-c3ccccc3)c3cc(-c4ccc(N(c5ccc6ccccc6c5)c5ccc6ccccc6c5)cc4)ccc3c3ccccc23)cc1]

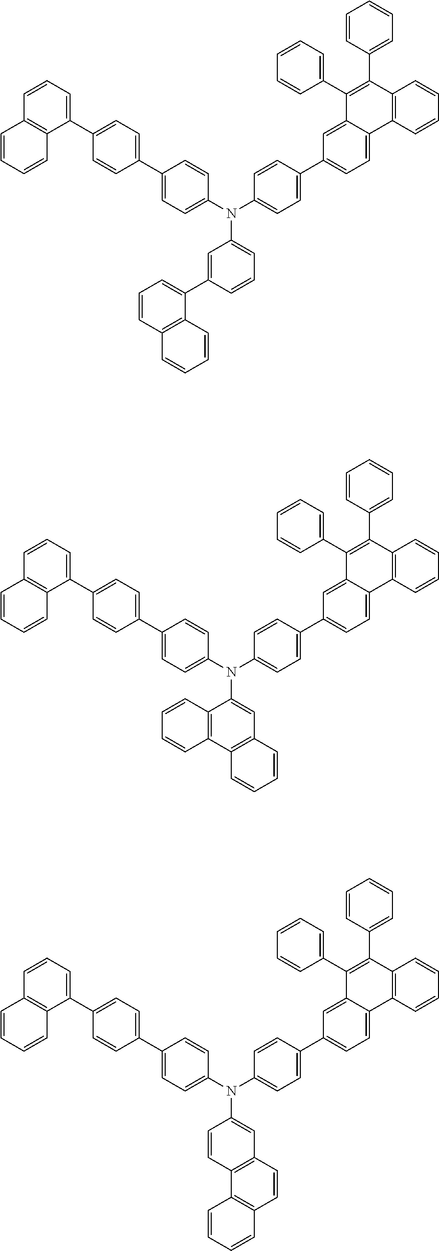 [SMILES: c1ccc(-c2c(-c3ccccc3)c3cc(-c4ccc(N(c5ccc(-c6ccc(-c7cccc8ccccc78)cc6)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cc6ccccc6c6ccccc56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc6c(ccc7ccccc76)c5)cc4)ccc3c3ccccc23)cc1]

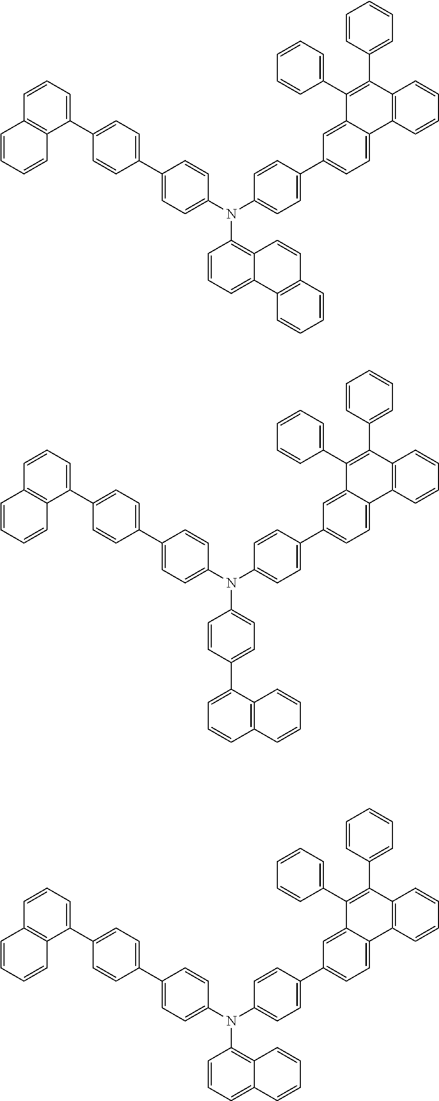 [SMILES: c1ccc(-c2c(-c3ccccc3)c3cc(-c4ccc(N(c5ccc(-c6ccc(-c7cccc8ccccc78)cc6)cc5)c5cccc6c5ccc5ccccc56)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5ccc(-c6cccc7ccccc67)cc5)cc4)ccc3c3ccccc23)cc1 ; c1ccc(-c2c(-c3ccccc3)c3cc(-c4ccc(N(c5ccc(-c6ccc(-c7cccc8ccccc78)cc6)cc5)c5cccc6ccccc56)cc4)ccc3c3ccccc23)cc1]

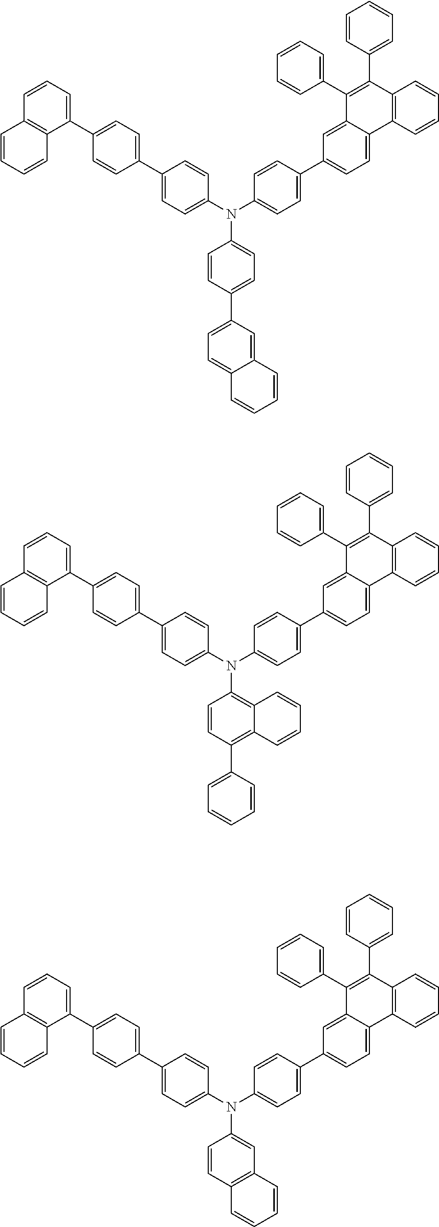 [SMILES: c1ccc(-c2c(-c3ccccc3)c3cc(-c4ccc(N(c5ccc(-c6ccc(-c7cccc8ccccc78)cc6)cc5)c5ccc(-c6ccc7ccccc7c6)cc5)cc4)ccc3c3ccccc23)cc1 ; c1ccc(-c2ccc(N(c3ccc(-c4ccc(-c5cccc6ccccc56)cc4)cc3)c3ccc(-c4ccc5c(c4)c(-c4ccccc4)c(-c4ccccc4)c4ccccc45)cc3)c3ccccc23)cc1 ; c1ccc(-c2c(-c3ccccc3)c3cc(-c4ccc(N(c5ccc(-c6ccc(-c7cccc8ccccc78)cc6)cc5)c5ccc6ccccc6c5)cc4)ccc3c3ccccc23)cc1]

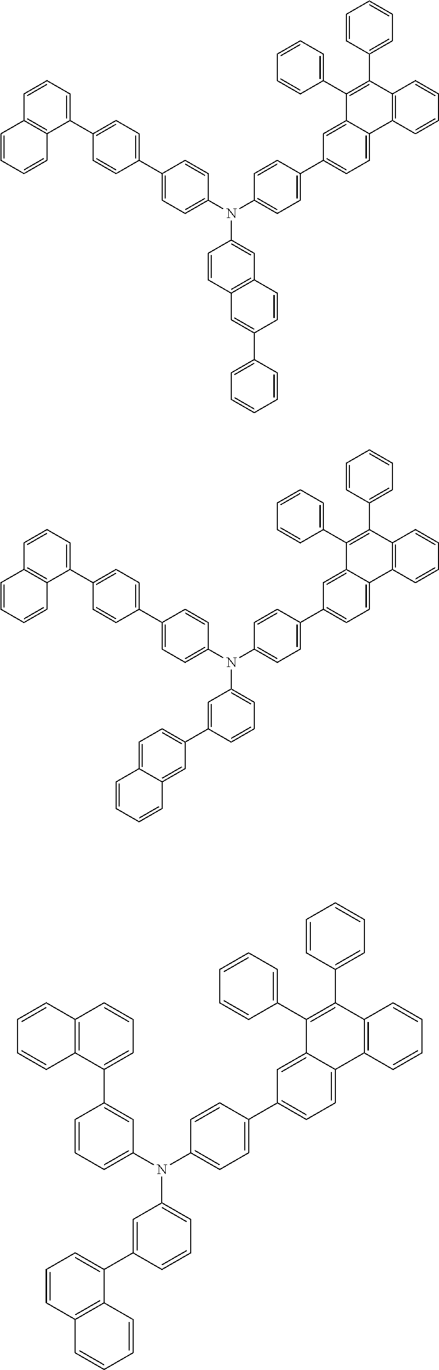 [SMILES: c1ccc(-c2ccc3cc(N(c4ccc(-c5ccc(-c6cccc7ccccc67)cc5)cc4)c4ccc(-c5ccc6c(c5)c(-c5ccccc5)c(-c5ccccc5)c5ccccc56)cc4)ccc3c2)cc1 ; c1ccc(-c2c(-c3ccccc3)c3cc(-c4ccc(N(c5ccc(-c6ccc(-c7cccc8ccccc78)cc6)cc5)c5cccc(-c6ccc7ccccc7c6)c5)cc4)ccc3c3ccccc23)cc1 ; c1ccc(-c2c(-c3ccccc3)c3cc(-c4ccc(N(c5cccc(-c6cccc7ccccc67)c5)c5cccc(-c6cccc7ccccc67)c5)cc4)ccc3c3ccccc23)cc1]

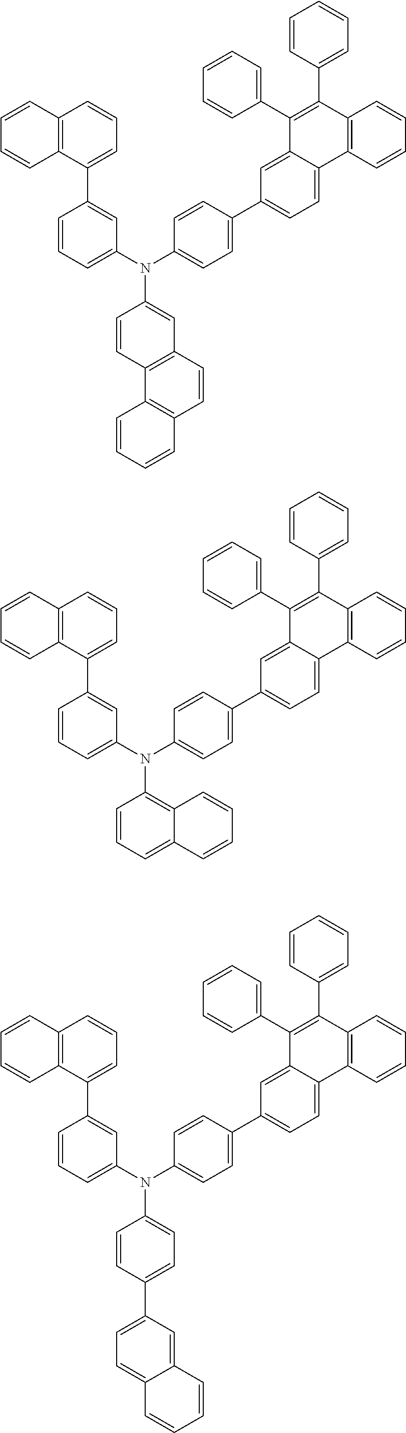 [SMILES: c1ccc(-c2c(-c3ccccc3)c3cc(-c4ccc(N(c5cccc(-c6cccc7ccccc67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7ccccc67)c5)c5cccc6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5cccc(-c6cccc7ccccc67)c5)cc4)ccc3c3ccccc23)cc1]

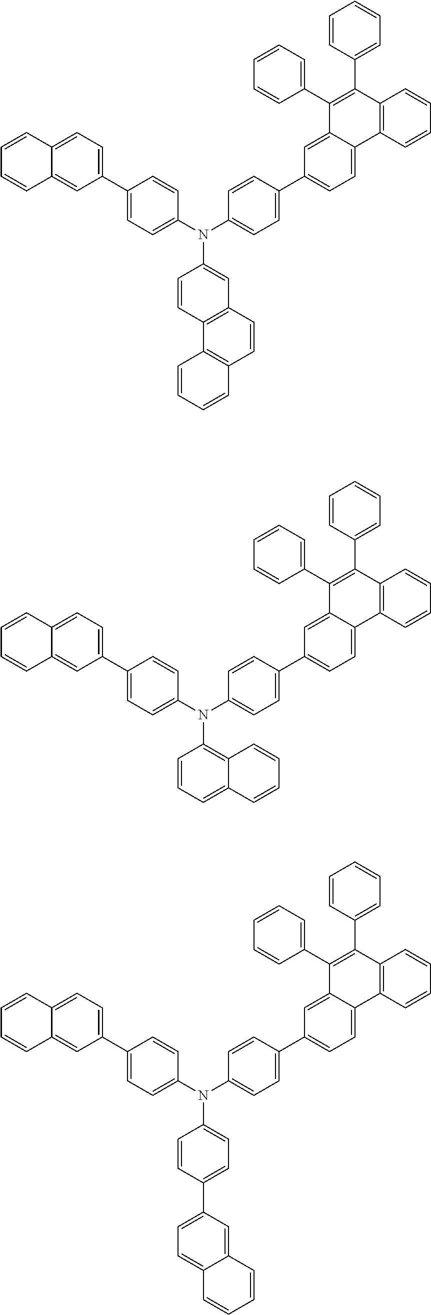 [SMILES: c1ccc(-c2c(-c3ccccc3)c3cc(-c4ccc(N(c5ccc(-c6ccc7ccccc7c6)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6ccc7ccccc7c6)cc5)c5cccc6ccccc56)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6ccc7ccccc7c6)cc5)cc4)ccc3c3ccccc23)cc1]

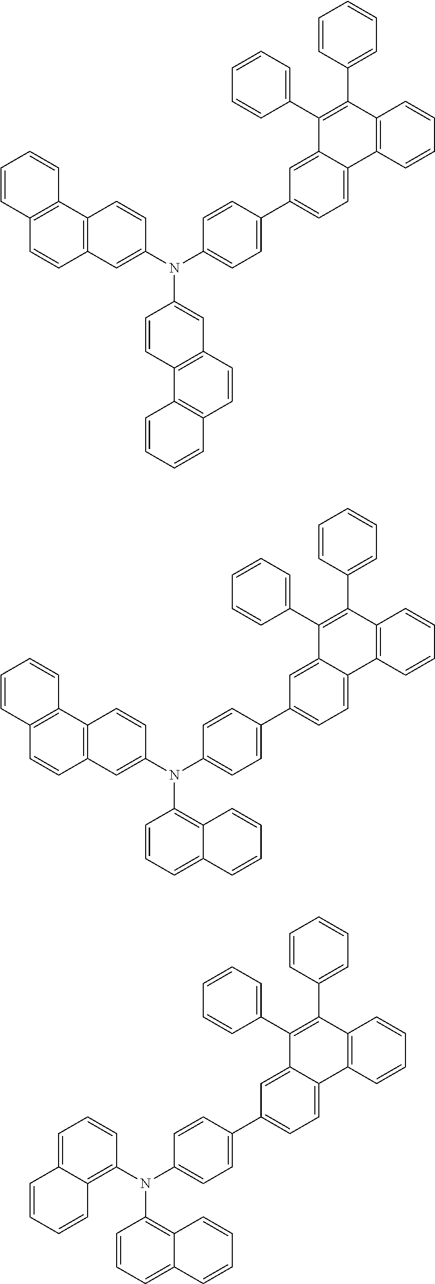 [SMILES: c1ccc(-c2c(-c3ccccc3)c3cc(-c4ccc(N(c5ccc6c(ccc7ccccc76)c5)c5ccc6c(ccc7ccccc76)c5)cc4)ccc3c3ccccc23)cc1 ; c1ccc(-c2c(-c3ccccc3)c3cc(-c4ccc(N(c5ccc6c(ccc7ccccc76)c5)c5cccc6ccccc56)cc4)ccc3c3ccccc23)cc1 ; c1ccc(-c2c(-c3ccccc3)c3cc(-c4ccc(N(c5cccc6ccccc56)c5cccc6ccccc56)cc4)ccc3c3ccccc23)cc1]

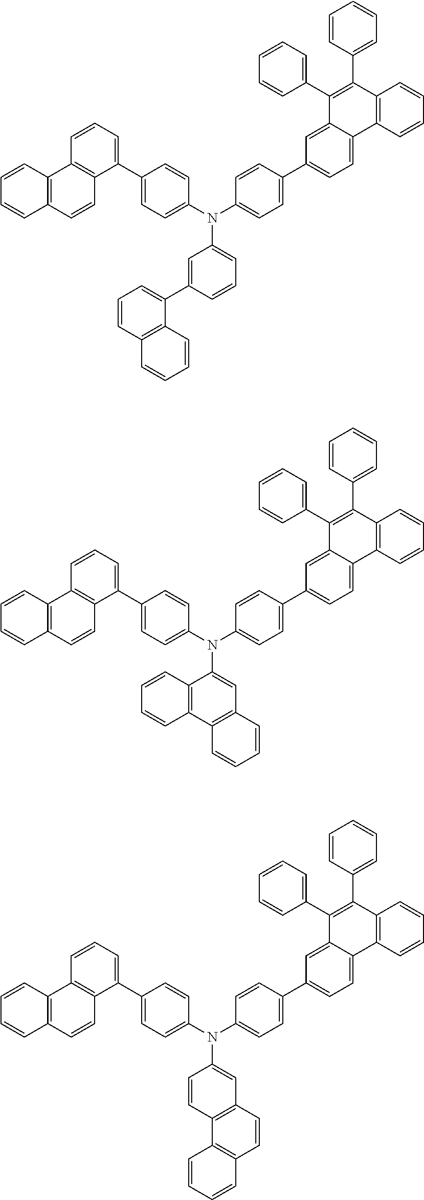 [SMILES: c1ccc(-c2c(-c3ccccc3)c3cc(-c4ccc(N(c5ccc(-c6cccc7c6ccc6ccccc67)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6cccc7c6ccc6ccccc67)cc5)c5cc6ccccc6c6ccccc56)cc4)ccc3c3ccccc23)cc1 ; c1ccc(-c2c(-c3ccccc3)c3cc(-c4ccc(N(c5ccc(-c6cccc7c6ccc6ccccc67)cc5)c5ccc6c(ccc7ccccc76)c5)cc4)ccc3c3ccccc23)cc1]

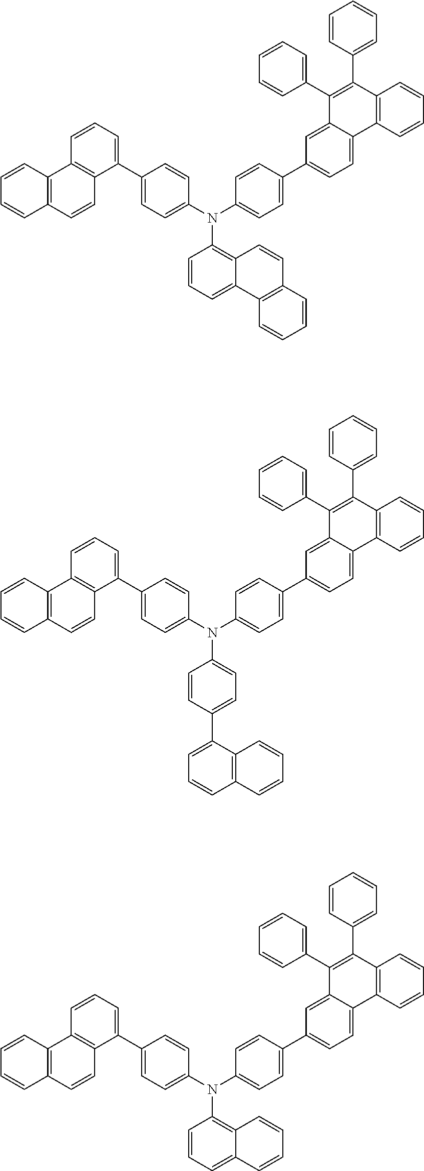 [SMILES: c1ccc(-c2c(-c3ccccc3)c3cc(-c4ccc(N(c5ccc(-c6cccc7c6ccc6ccccc67)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc(-c6cccc7c6ccc6ccccc67)cc5)cc4)ccc3c3ccccc23)cc1 ; c1ccc(-c2c(-c3ccccc3)c3cc(-c4ccc(N(c5ccc(-c6cccc7c6ccc6ccccc67)cc5)c5cccc6ccccc56)cc4)ccc3c3ccccc23)cc1]

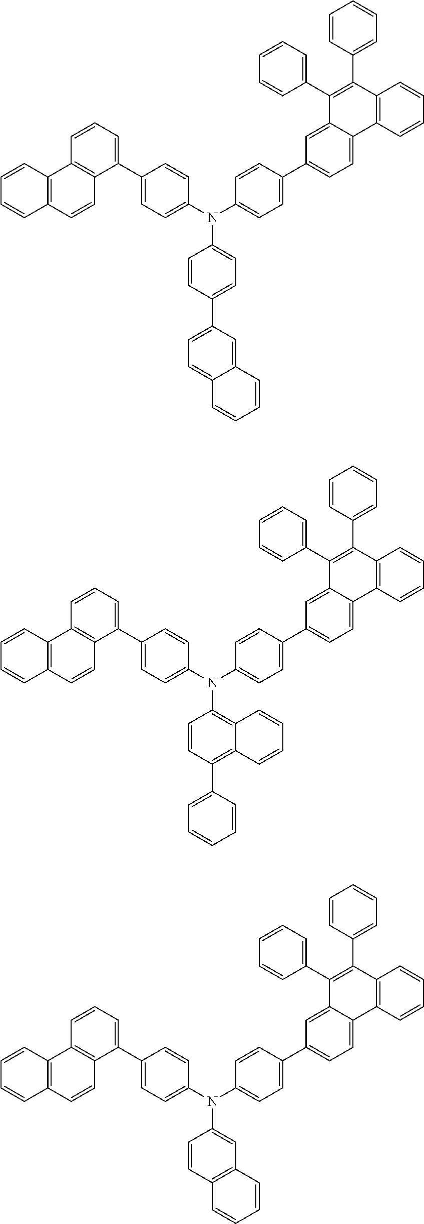 [SMILES: c1ccc(-c2c(-c3ccccc3)c3cc(-c4ccc(N(c5ccc(-c6ccc7ccccc7c6)cc5)c5ccc(-c6cccc7c6ccc6ccccc67)cc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc(-c4cccc5c4ccc4ccccc45)cc3)c3ccccc23)cc1 ; c1ccc(-c2c(-c3ccccc3)c3cc(-c4ccc(N(c5ccc(-c6cccc7c6ccc6ccccc67)cc5)c5ccc6ccccc6c5)cc4)ccc3c3ccccc23)cc1]

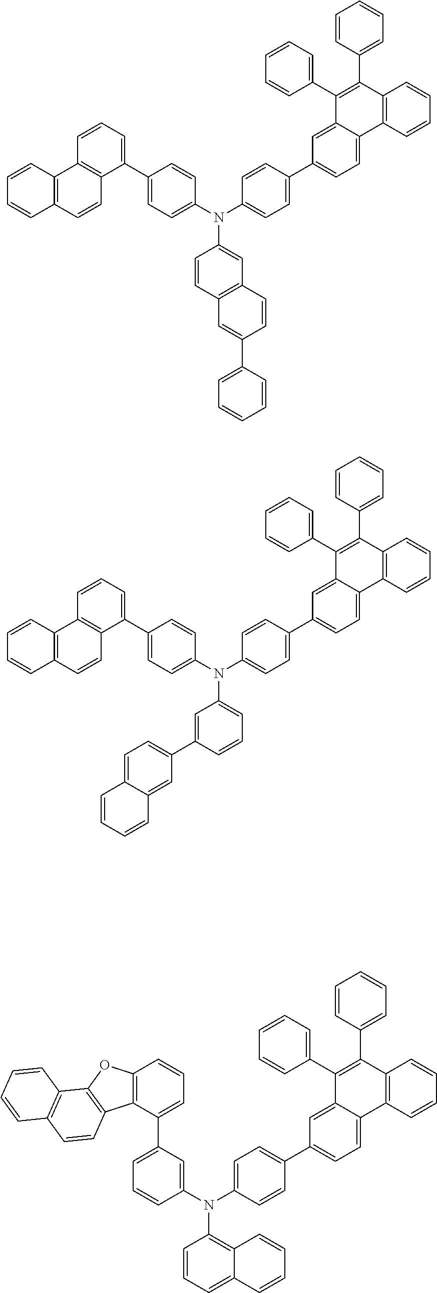 [SMILES: c1ccc(-c2ccc3cc(N(c4ccc(-c5ccc6c(c5)c(-c5ccccc5)c(-c5ccccc5)c5ccccc56)cc4)c4ccc(-c5cccc6c5ccc5ccccc56)cc4)ccc3c2)cc1 ; c1ccc(-c2c(-c3ccccc3)c3cc(-c4ccc(N(c5ccc(-c6cccc7c6ccc6ccccc67)cc5)c5cccc(-c6ccc7ccccc7c6)c5)cc4)ccc3c3ccccc23)cc1 ; c1ccc(-c2c(-c3ccccc3)c3cc(-c4ccc(N(c5cccc(-c6cccc7oc8c9ccccc9ccc8c67)c5)c5cccc6ccccc56)cc4)ccc3c3ccccc23)cc1]

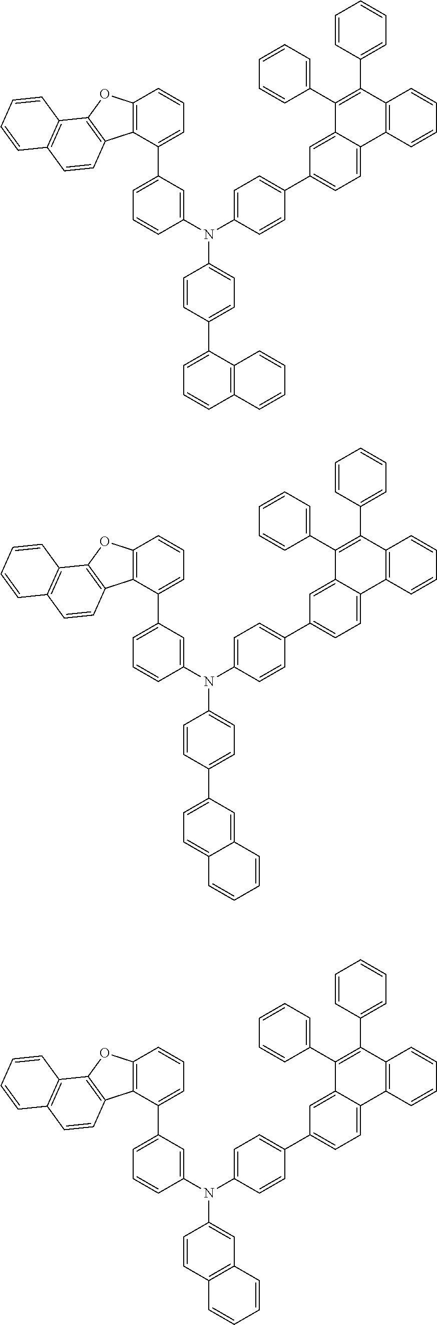 [SMILES: c1ccc(-c2c(-c3ccccc3)c3cc(-c4ccc(N(c5ccc(-c6cccc7ccccc67)cc5)c5cccc(-c6cccc7oc8c9ccccc9ccc8c67)c5)cc4)ccc3c3ccccc23)cc1 ; c1ccc(-c2c(-c3ccccc3)c3cc(-c4ccc(N(c5ccc(-c6ccc7ccccc7c6)cc5)c5cccc(-c6cccc7oc8c9ccccc9ccc8c67)c5)cc4)ccc3c3ccccc23)cc1 ; c1ccc(-c2c(-c3ccccc3)c3cc(-c4ccc(N(c5cccc(-c6cccc7oc8c9ccccc9ccc8c67)c5)c5ccc6ccccc6c5)cc4)ccc3c3ccccc23)cc1]

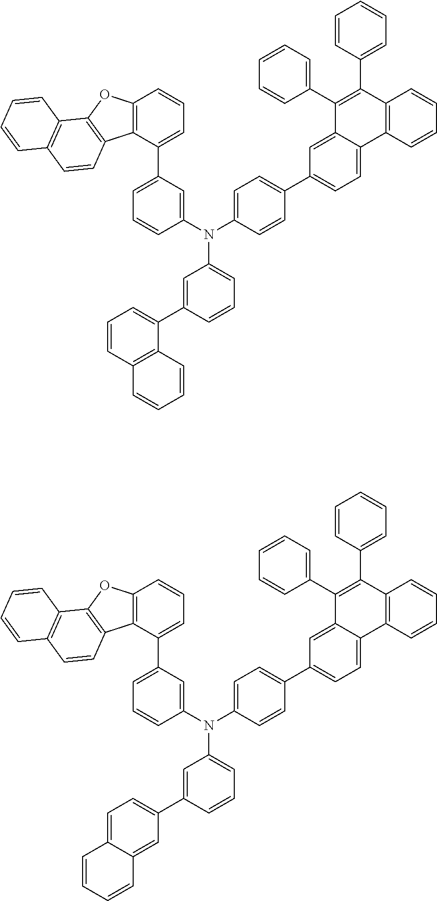 [SMILES: c1ccc(-c2c(-c3ccccc3)c3cc(-c4ccc(N(c5cccc(-c6cccc7ccccc67)c5)c5cccc(-c6cccc7oc8c9ccccc9ccc8c67)c5)cc4)ccc3c3ccccc23)cc1 ; c1ccc(-c2c(-c3ccccc3)c3cc(-c4ccc(N(c5cccc(-c6ccc7ccccc7c6)c5)c5cccc(-c6cccc7oc8c9ccccc9ccc8c67)c5)cc4)ccc3c3ccccc23)cc1]

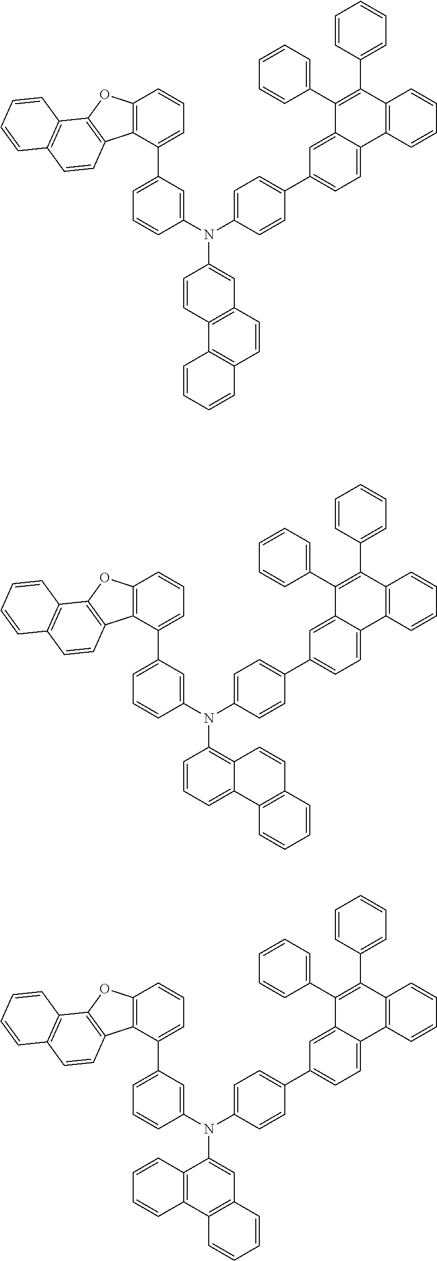 [SMILES: c1ccc(-c2c(-c3ccccc3)c3cc(-c4ccc(N(c5cccc(-c6cccc7oc8c9ccccc9ccc8c67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7oc8c9ccccc9ccc8c67)c5)c5cccc6c5ccc5ccccc56)cc4)ccc3c3ccccc23)cc1 ; c1ccc(-c2c(-c3ccccc3)c3cc(-c4ccc(N(c5cccc(-c6cccc7oc8c9ccccc9ccc8c67)c5)c5cc6ccccc6c6ccccc56)cc4)ccc3c3ccccc23)cc1]

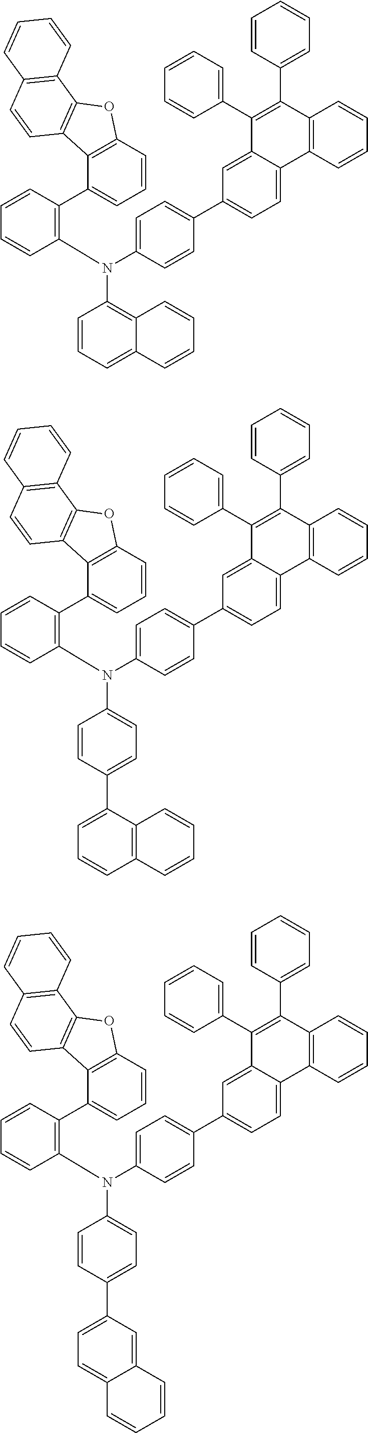 [SMILES: c1ccc(-c2c(-c3ccccc3)c3cc(-c4ccc(N(c5ccccc5-c5cccc6oc7c8ccccc8ccc7c56)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccccc5-c5cccc6oc7c8ccccc8ccc7c56)cc4)ccc3c3ccccc23)cc1 ; c1ccc(-c2c(-c3ccccc3)c3cc(-c4ccc(N(c5ccc(-c6ccc7ccccc7c6)cc5)c5ccccc5-c5cccc6oc7c8ccccc8ccc7c56)cc4)ccc3c3ccccc23)cc1]

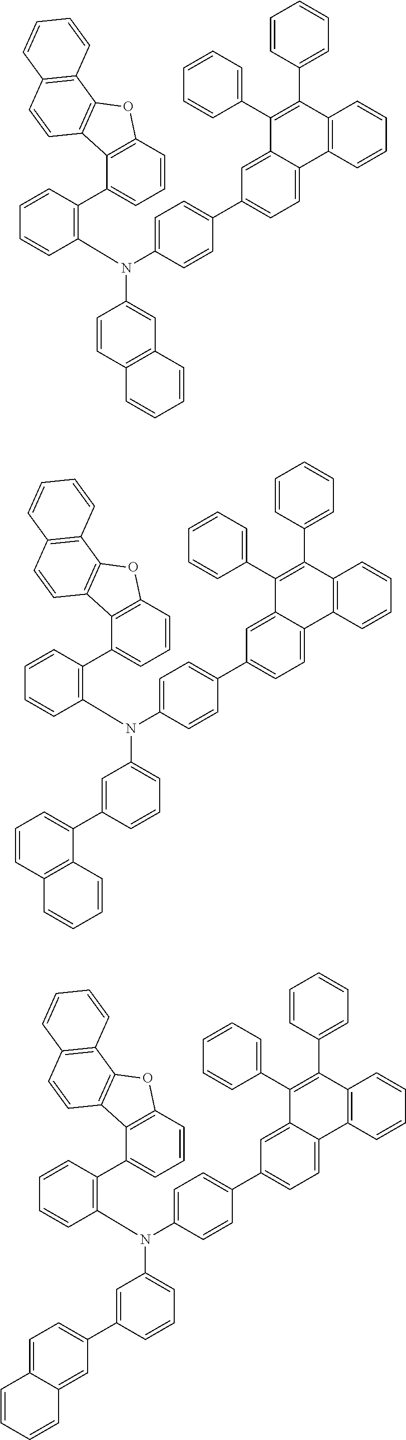 [SMILES: c1ccc(-c2c(-c3ccccc3)c3cc(-c4ccc(N(c5ccc6ccccc6c5)c5ccccc5-c5cccc6oc7c8ccccc8ccc7c56)cc4)ccc3c3ccccc23)cc1 ; c1ccc(-c2c(-c3ccccc3)c3cc(-c4ccc(N(c5cccc(-c6cccc7ccccc67)c5)c5ccccc5-c5cccc6oc7c8ccccc8ccc7c56)cc4)ccc3c3ccccc23)cc1 ; c1ccc(-c2c(-c3ccccc3)c3cc(-c4ccc(N(c5cccc(-c6ccc7ccccc7c6)c5)c5ccccc5-c5cccc6oc7c8ccccc8ccc7c56)cc4)ccc3c3ccccc23)cc1]

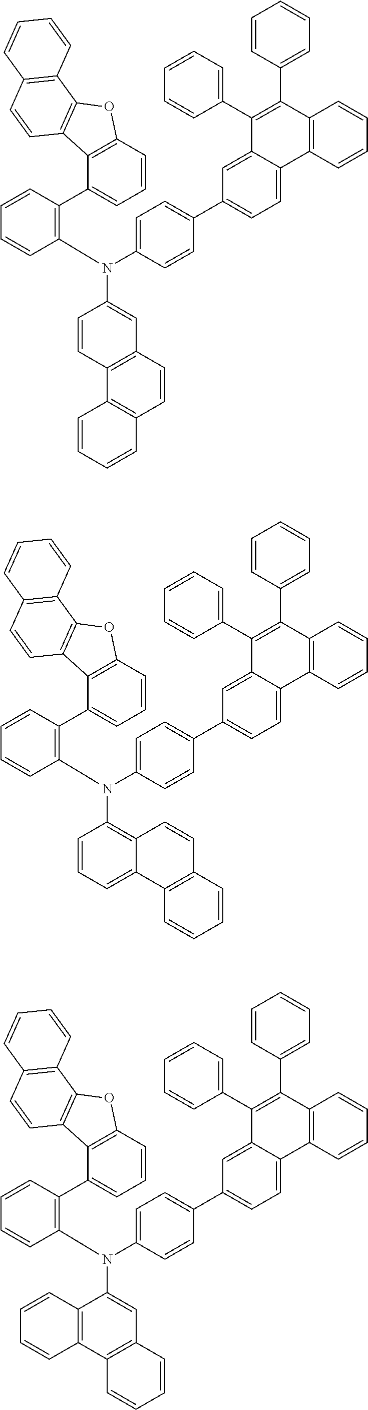 [SMILES: c1ccc(-c2c(-c3ccccc3)c3cc(-c4ccc(N(c5ccc6c(ccc7ccccc76)c5)c5ccccc5-c5cccc6oc7c8ccccc8ccc7c56)cc4)ccc3c3ccccc23)cc1 ; c1ccc(-c2c(-c3ccccc3)c3cc(-c4ccc(N(c5ccccc5-c5cccc6oc7c8ccccc8ccc7c56)c5cccc6c5ccc5ccccc56)cc4)ccc3c3ccccc23)cc1 ; c1ccc(-c2c(-c3ccccc3)c3cc(-c4ccc(N(c5ccccc5-c5cccc6oc7c8ccccc8ccc7c56)c5cc6ccccc6c6ccccc56)cc4)ccc3c3ccccc23)cc1]

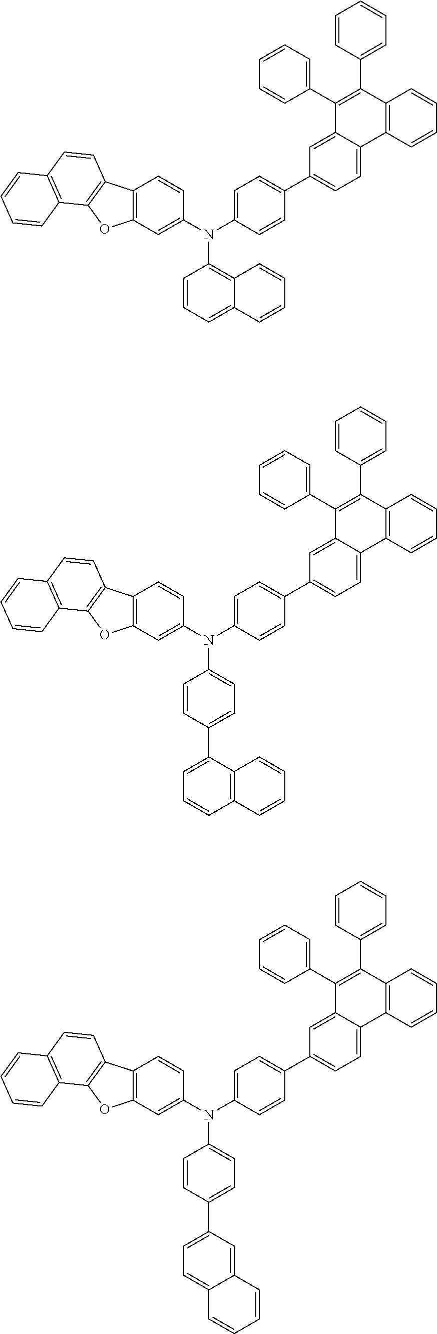 [SMILES: c1ccc(-c2c(-c3ccccc3)c3cc(-c4ccc(N(c5ccc6c(c5)oc5c7ccccc7ccc65)c5cccc6ccccc56)cc4)ccc3c3ccccc23)cc1 ; c1ccc(-c2c(-c3ccccc3)c3cc(-c4ccc(N(c5ccc(-c6cccc7ccccc67)cc5)c5ccc6c(c5)oc5c7ccccc7ccc65)cc4)ccc3c3ccccc23)cc1 ; c1ccc(-c2c(-c3ccccc3)c3cc(-c4ccc(N(c5ccc(-c6ccc7ccccc7c6)cc5)c5ccc6c(c5)oc5c7ccccc7ccc65)cc4)ccc3c3ccccc23)cc1]

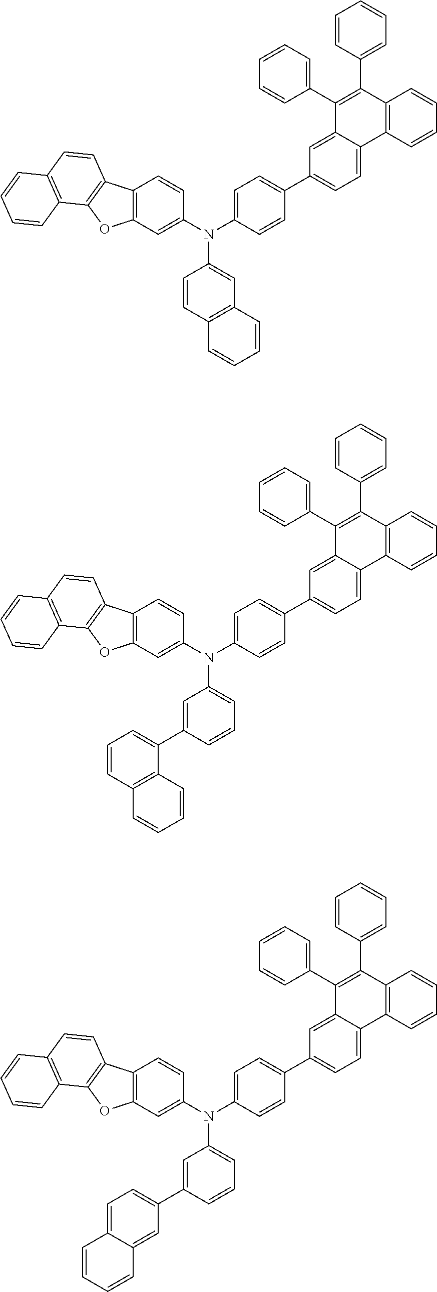 [SMILES: c1ccc(-c2c(-c3ccccc3)c3cc(-c4ccc(N(c5ccc6ccccc6c5)c5ccc6c(c5)oc5c7ccccc7ccc65)cc4)ccc3c3ccccc23)cc1 ; c1ccc(-c2c(-c3ccccc3)c3cc(-c4ccc(N(c5cccc(-c6cccc7ccccc67)c5)c5ccc6c(c5)oc5c7ccccc7ccc65)cc4)ccc3c3ccccc23)cc1 ; c1ccc(-c2c(-c3ccccc3)c3cc(-c4ccc(N(c5cccc(-c6ccc7ccccc7c6)c5)c5ccc6c(c5)oc5c7ccccc7ccc65)cc4)ccc3c3ccccc23)cc1]

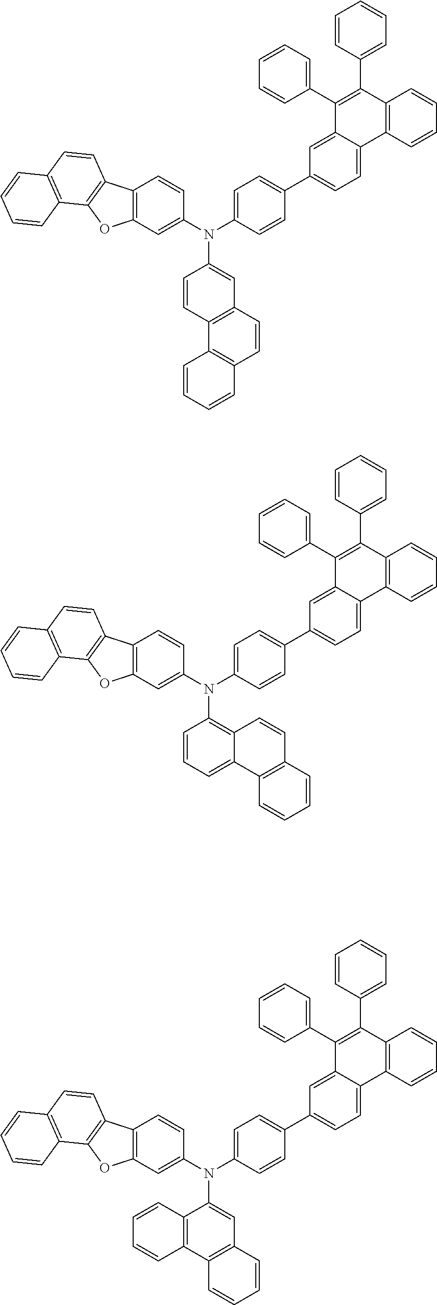 [SMILES: c1ccc(-c2c(-c3ccccc3)c3cc(-c4ccc(N(c5ccc6c(ccc7ccccc76)c5)c5ccc6c(c5)oc5c7ccccc7ccc65)cc4)ccc3c3ccccc23)cc1 ; c1ccc(-c2c(-c3ccccc3)c3cc(-c4ccc(N(c5ccc6c(c5)oc5c7ccccc7ccc65)c5cccc6c5ccc5ccccc56)cc4)ccc3c3ccccc23)cc1 ; c1ccc(-c2c(-c3ccccc3)c3cc(-c4ccc(N(c5ccc6c(c5)oc5c7ccccc7ccc65)c5cc6ccccc6c6ccccc56)cc4)ccc3c3ccccc23)cc1]

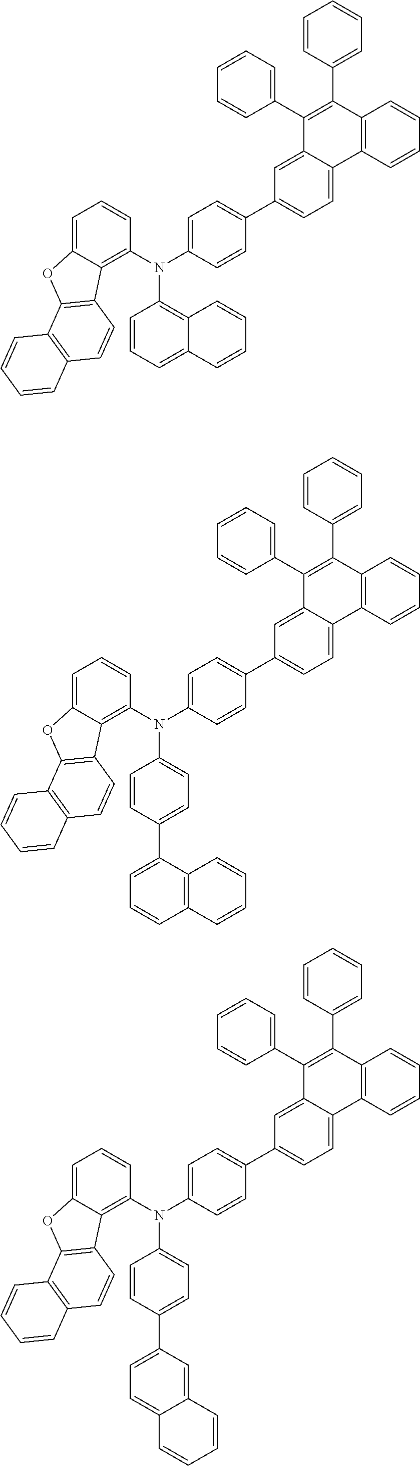 [SMILES: c1ccc(-c2c(-c3ccccc3)c3cc(-c4ccc(N(c5cccc6ccccc56)c5cccc6oc7c8ccccc8ccc7c56)cc4)ccc3c3ccccc23)cc1 ; c1ccc(-c2c(-c3ccccc3)c3cc(-c4ccc(N(c5ccc(-c6cccc7ccccc67)cc5)c5cccc6oc7c8ccccc8ccc7c56)cc4)ccc3c3ccccc23)cc1 ; c1ccc(-c2c(-c3ccccc3)c3cc(-c4ccc(N(c5ccc(-c6ccc7ccccc7c6)cc5)c5cccc6oc7c8ccccc8ccc7c56)cc4)ccc3c3ccccc23)cc1]

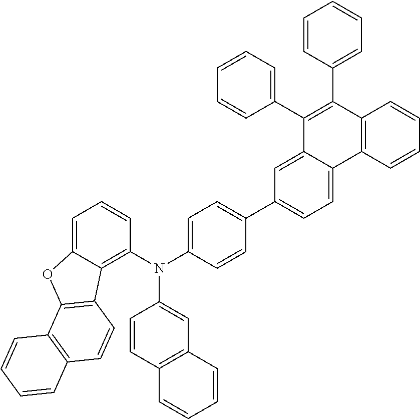 [SMILES: c1ccc(-c2c(-c3ccccc3)c3cc(-c4ccc(N(c5ccc6ccccc6c5)c5cccc6oc7c8ccccc8ccc7c56)cc4)ccc3c3ccccc23)cc1]

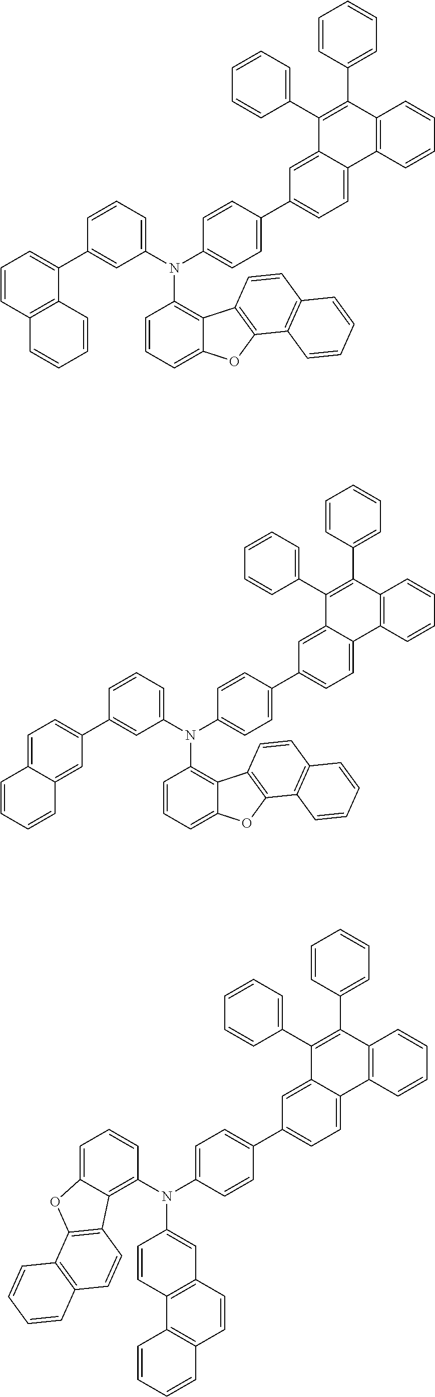 [SMILES: c1ccc(-c2c(-c3ccccc3)c3cc(-c4ccc(N(c5cccc(-c6cccc7ccccc67)c5)c5cccc6oc7c8ccccc8ccc7c56)cc4)ccc3c3ccccc23)cc1 ; c1ccc(-c2c(-c3ccccc3)c3cc(-c4ccc(N(c5cccc(-c6ccc7ccccc7c6)c5)c5cccc6oc7c8ccccc8ccc7c56)cc4)ccc3c3ccccc23)cc1 ; c1ccc(-c2c(-c3ccccc3)c3cc(-c4ccc(N(c5ccc6c(ccc7ccccc76)c5)c5cccc6oc7c8ccccc8ccc7c56)cc4)ccc3c3ccccc23)cc1]

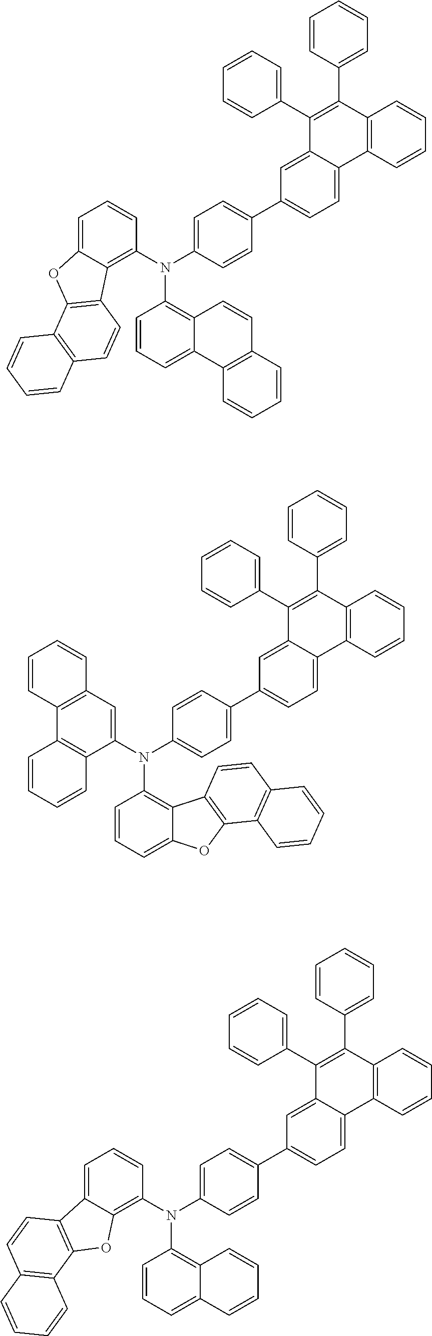 [SMILES: c1ccc(-c2c(-c3ccccc3)c3cc(-c4ccc(N(c5cccc6c5ccc5ccccc56)c5cccc6oc7c8ccccc8ccc7c56)cc4)ccc3c3ccccc23)cc1 ; c1ccc(-c2c(-c3ccccc3)c3cc(-c4ccc(N(c5cc6ccccc6c6ccccc56)c5cccc6oc7c8ccccc8ccc7c56)cc4)ccc3c3ccccc23)cc1 ; c1ccc(-c2c(-c3ccccc3)c3cc(-c4ccc(N(c5cccc6ccccc56)c5cccc6c5oc5c7ccccc7ccc65)cc4)ccc3c3ccccc23)cc1]

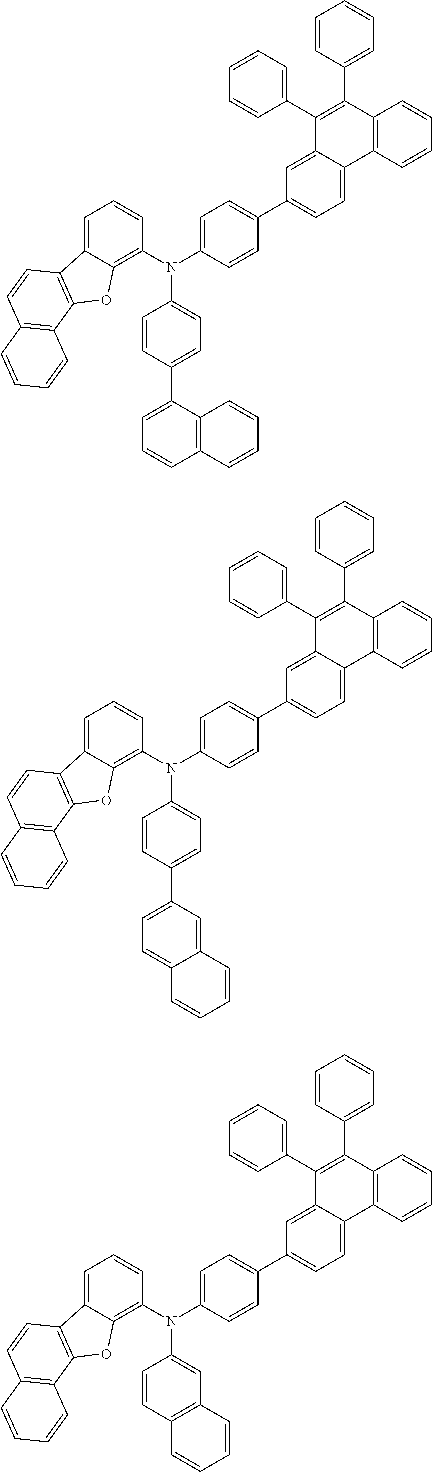 [SMILES: c1ccc(-c2c(-c3ccccc3)c3cc(-c4ccc(N(c5ccc(-c6cccc7ccccc67)cc5)c5cccc6c5oc5c7ccccc7ccc65)cc4)ccc3c3ccccc23)cc1 ; c1ccc(-c2c(-c3ccccc3)c3cc(-c4ccc(N(c5ccc(-c6ccc7ccccc7c6)cc5)c5cccc6c5oc5c7ccccc7ccc65)cc4)ccc3c3ccccc23)cc1 ; c1ccc(-c2c(-c3ccccc3)c3cc(-c4ccc(N(c5ccc6ccccc6c5)c5cccc6c5oc5c7ccccc7ccc65)cc4)ccc3c3ccccc23)cc1]

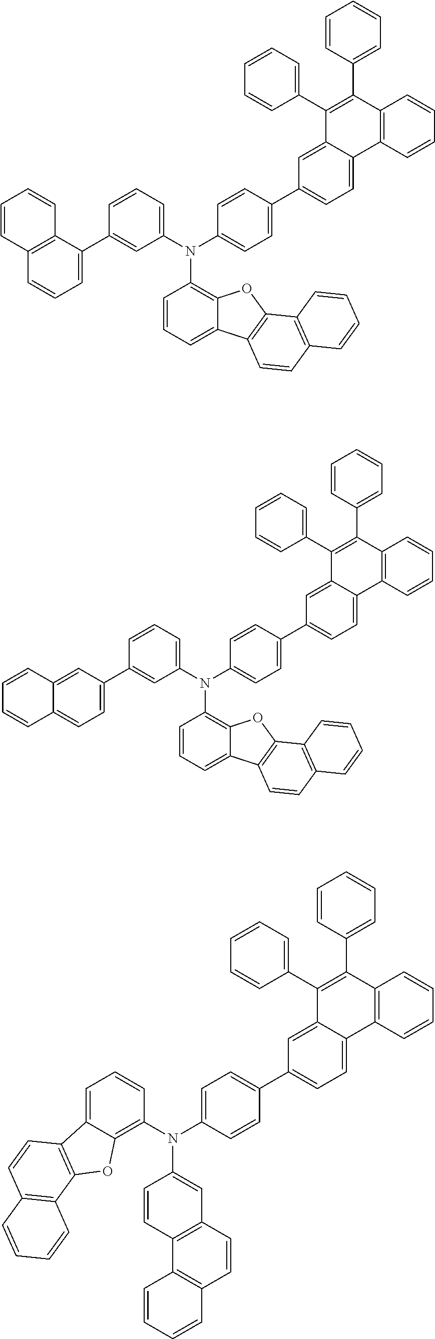 [SMILES: c1ccc(-c2c(-c3ccccc3)c3cc(-c4ccc(N(c5cccc(-c6cccc7ccccc67)c5)c5cccc6c5oc5c7ccccc7ccc65)cc4)ccc3c3ccccc23)cc1 ; c1ccc(-c2c(-c3ccccc3)c3cc(-c4ccc(N(c5cccc(-c6ccc7ccccc7c6)c5)c5cccc6c5oc5c7ccccc7ccc65)cc4)ccc3c3ccccc23)cc1 ; c1ccc(-c2c(-c3ccccc3)c3cc(-c4ccc(N(c5ccc6c(ccc7ccccc76)c5)c5cccc6c5oc5c7ccccc7ccc65)cc4)ccc3c3ccccc23)cc1]

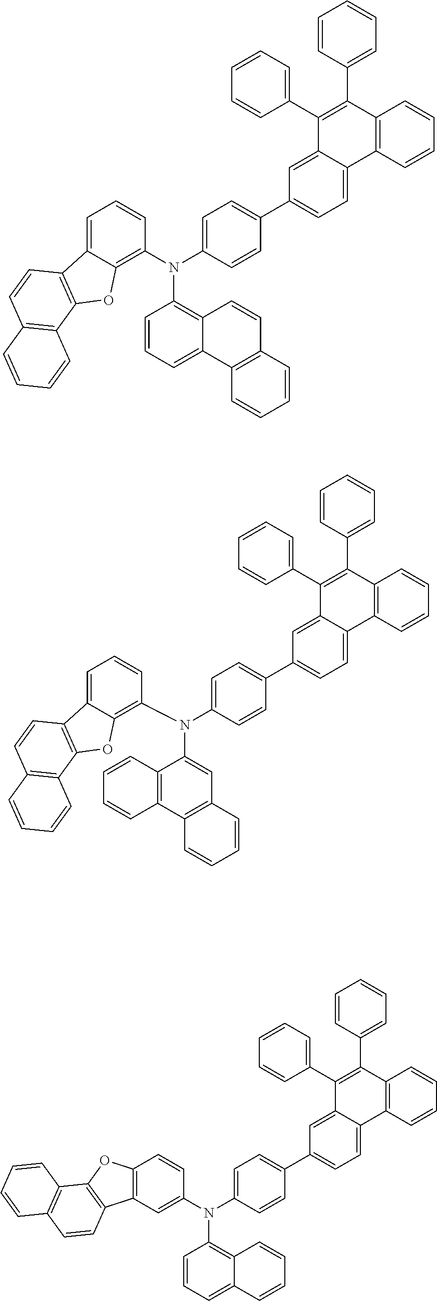 [SMILES: c1ccc(-c2c(-c3ccccc3)c3cc(-c4ccc(N(c5cccc6c5ccc5ccccc56)c5cccc6c5oc5c7ccccc7ccc65)cc4)ccc3c3ccccc23)cc1 ; c1ccc(-c2c(-c3ccccc3)c3cc(-c4ccc(N(c5cc6ccccc6c6ccccc56)c5cccc6c5oc5c7ccccc7ccc65)cc4)ccc3c3ccccc23)cc1 ; c1ccc(-c2c(-c3ccccc3)c3cc(-c4ccc(N(c5ccc6oc7c8ccccc8ccc7c6c5)c5cccc6ccccc56)cc4)ccc3c3ccccc23)cc1]

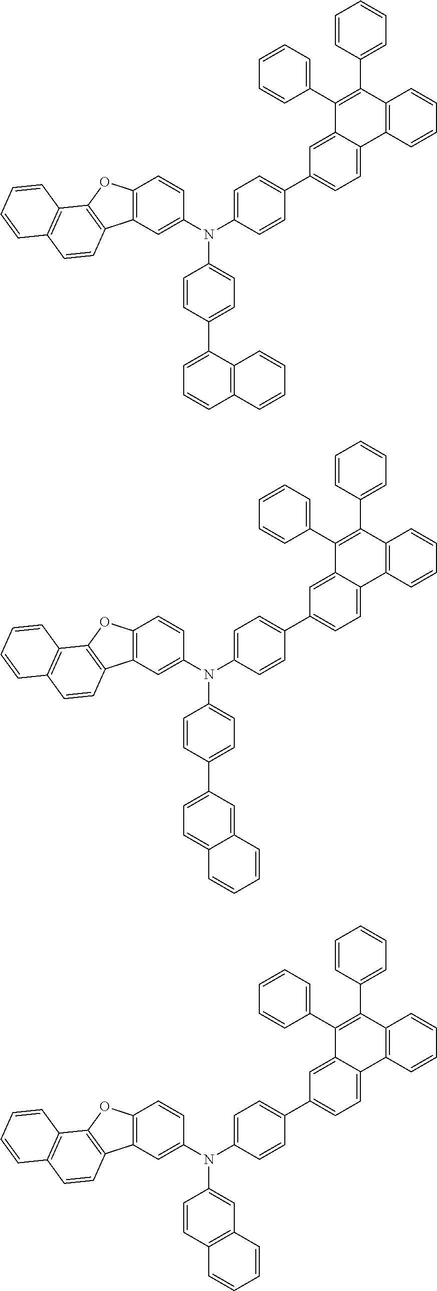 [SMILES: c1ccc(-c2c(-c3ccccc3)c3cc(-c4ccc(N(c5ccc(-c6cccc7ccccc67)cc5)c5ccc6oc7c8ccccc8ccc7c6c5)cc4)ccc3c3ccccc23)cc1 ; c1ccc(-c2c(-c3ccccc3)c3cc(-c4ccc(N(c5ccc(-c6ccc7ccccc7c6)cc5)c5ccc6oc7c8ccccc8ccc7c6c5)cc4)ccc3c3ccccc23)cc1 ; c1ccc(-c2c(-c3ccccc3)c3cc(-c4ccc(N(c5ccc6ccccc6c5)c5ccc6oc7c8ccccc8ccc7c6c5)cc4)ccc3c3ccccc23)cc1]

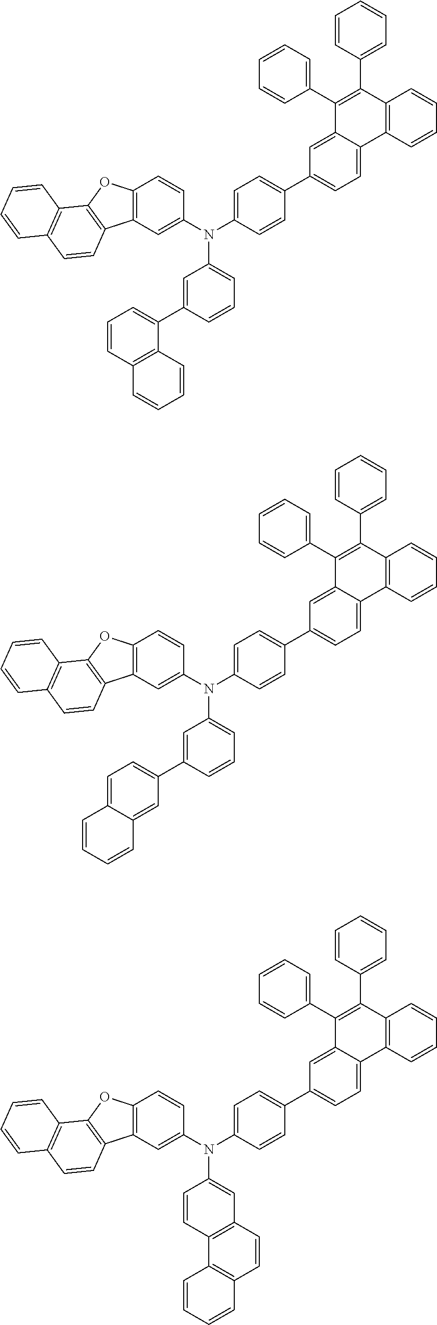 [SMILES: c1ccc(-c2c(-c3ccccc3)c3cc(-c4ccc(N(c5cccc(-c6cccc7ccccc67)c5)c5ccc6oc7c8ccccc8ccc7c6c5)cc4)ccc3c3ccccc23)cc1 ; c1ccc(-c2c(-c3ccccc3)c3cc(-c4ccc(N(c5cccc(-c6ccc7ccccc7c6)c5)c5ccc6oc7c8ccccc8ccc7c6c5)cc4)ccc3c3ccccc23)cc1 ; c1ccc(-c2c(-c3ccccc3)c3cc(-c4ccc(N(c5ccc6c(ccc7ccccc76)c5)c5ccc6oc7c8ccccc8ccc7c6c5)cc4)ccc3c3ccccc23)cc1]

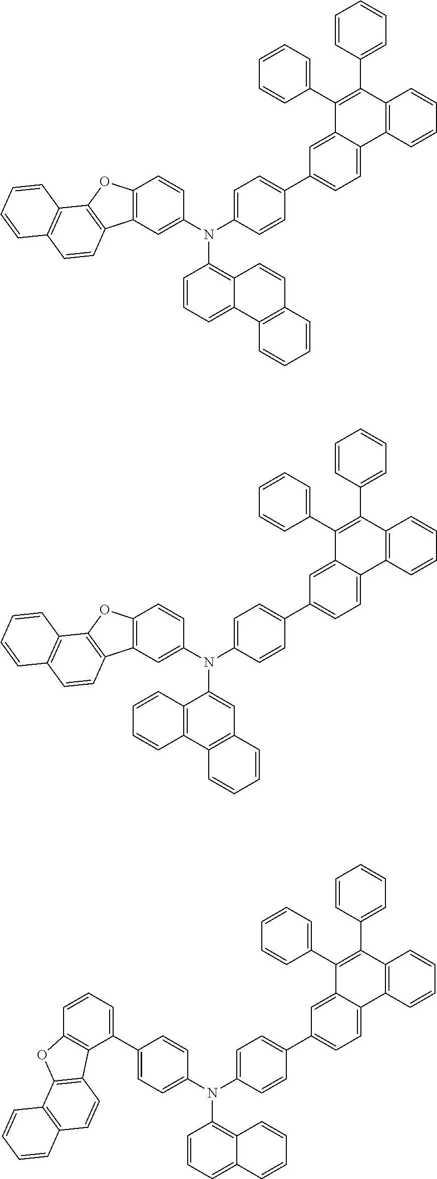 [SMILES: c1ccc(-c2c(-c3ccccc3)c3cc(-c4ccc(N(c5ccc6oc7c8ccccc8ccc7c6c5)c5cccc6c5ccc5ccccc56)cc4)ccc3c3ccccc23)cc1 ; c1ccc(-c2c(-c3ccccc3)c3cc(-c4ccc(N(c5ccc6oc7c8ccccc8ccc7c6c5)c5cc6ccccc6c6ccccc56)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5cccc6ccccc56)cc4)ccc3c3ccccc23)cc1]

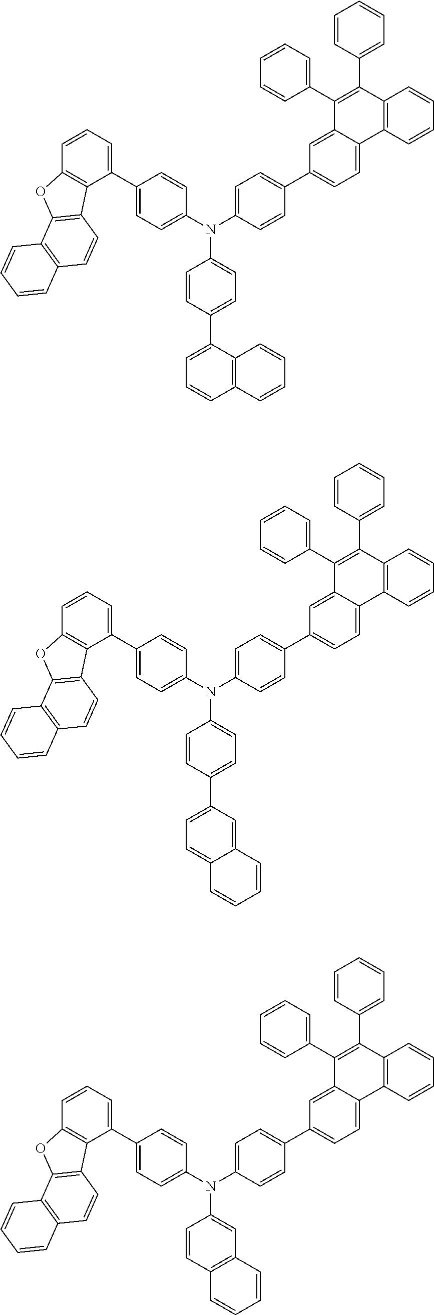 [SMILES: c1ccc(-c2c(-c3ccccc3)c3cc(-c4ccc(N(c5ccc(-c6cccc7ccccc67)cc5)c5ccc(-c6cccc7oc8c9ccccc9ccc8c67)cc5)cc4)ccc3c3ccccc23)cc1 ; c1ccc(-c2c(-c3ccccc3)c3cc(-c4ccc(N(c5ccc(-c6ccc7ccccc7c6)cc5)c5ccc(-c6cccc7oc8c9ccccc9ccc8c67)cc5)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5ccc6ccccc6c5)cc4)ccc3c3ccccc23)cc1]

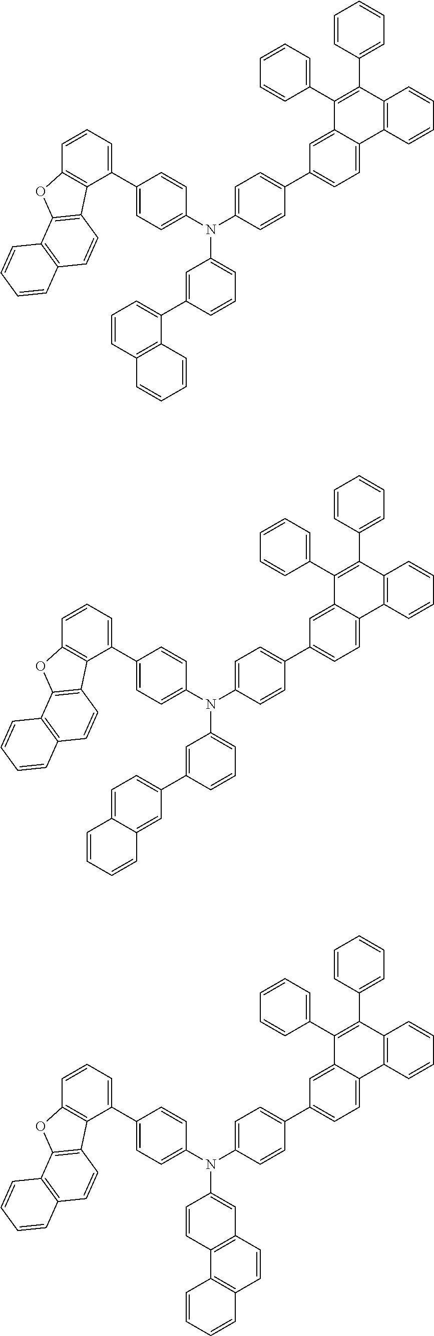 [SMILES: c1ccc(-c2c(-c3ccccc3)c3cc(-c4ccc(N(c5ccc(-c6cccc7oc8c9ccccc9ccc8c67)cc5)c5cccc(-c6cccc7ccccc67)c5)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5cccc(-c6ccc7ccccc7c6)c5)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5ccc6c(ccc7ccccc76)c5)cc4)ccc3c3ccccc23)cc1]

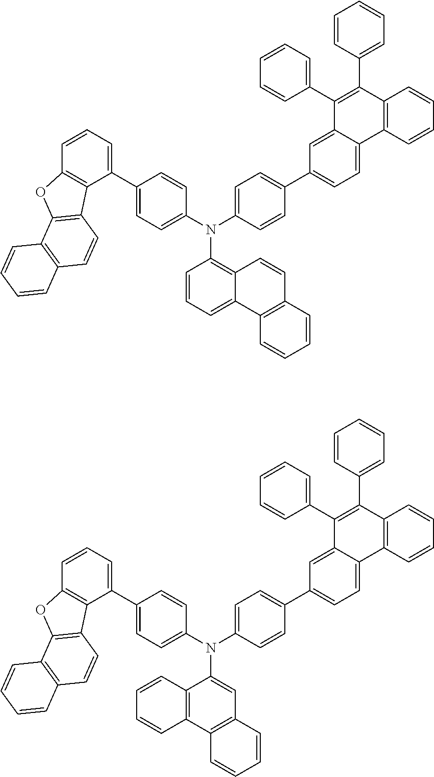 [SMILES: c1ccc(-c2c(-c3ccccc3)c3cc(-c4ccc(N(c5ccc(-c6cccc7oc8c9ccccc9ccc8c67)cc5)c5cccc6c5ccc5ccccc56)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5cc6ccccc6c6ccccc56)cc4)ccc3c3ccccc23)cc1]

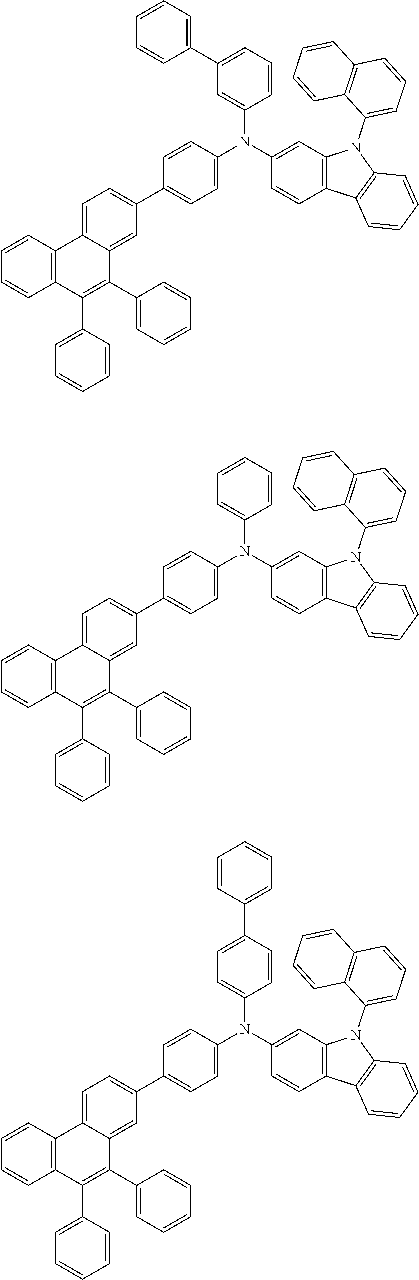 [SMILES: c1ccc(-c2cccc(N(c3ccc(-c4ccc5c(c4)c(-c4ccccc4)c(-c4ccccc4)c4ccccc45)cc3)c3ccc4c5ccccc5n(-c5cccc6ccccc56)c4c3)c2)cc1 ; c1ccc(-c2c(-c3ccccc3)c3cc(-c4ccc(N(c5ccccc5)c5ccc6c7ccccc7n(-c7cccc8ccccc78)c6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c5ccccc5n(-c5cccc6ccccc56)c4c3)cc2)cc1]

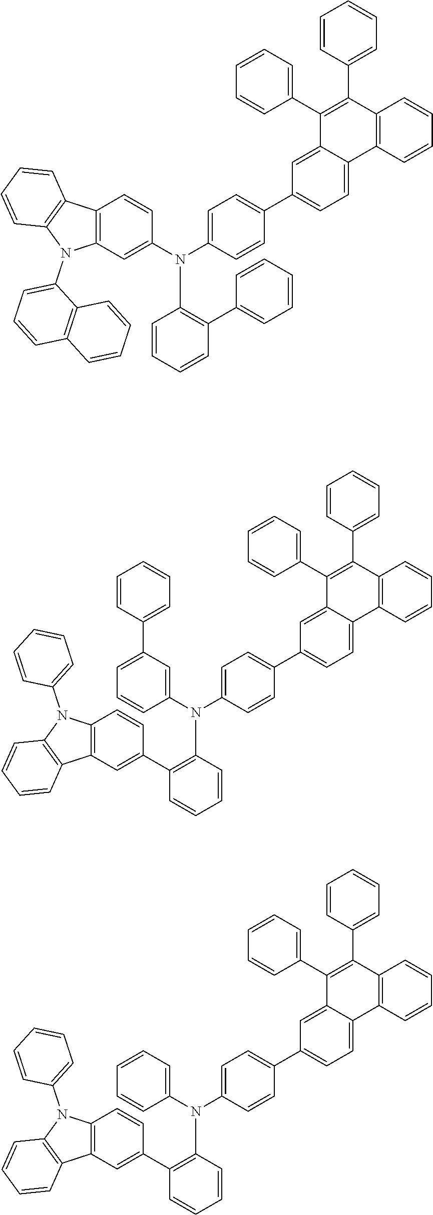 [SMILES: c1ccc(-c2ccccc2N(c2ccc(-c3ccc4c(c3)c(-c3ccccc3)c(-c3ccccc3)c3ccccc34)cc2)c2ccc3c4ccccc4n(-c4cccc5ccccc45)c3c2)cc1 ; c1ccc(-c2cccc(N(c3ccc(-c4ccc5c(c4)c(-c4ccccc4)c(-c4ccccc4)c4ccccc45)cc3)c3ccccc3-c3ccc4c(c3)c3ccccc3n4-c3ccccc3)c2)cc1 ; c1ccc(-c2c(-c3ccccc3)c3cc(-c4ccc(N(c5ccccc5)c5ccccc5-c5ccc6c(c5)c5ccccc5n6-c5ccccc5)cc4)ccc3c3ccccc23)cc1]

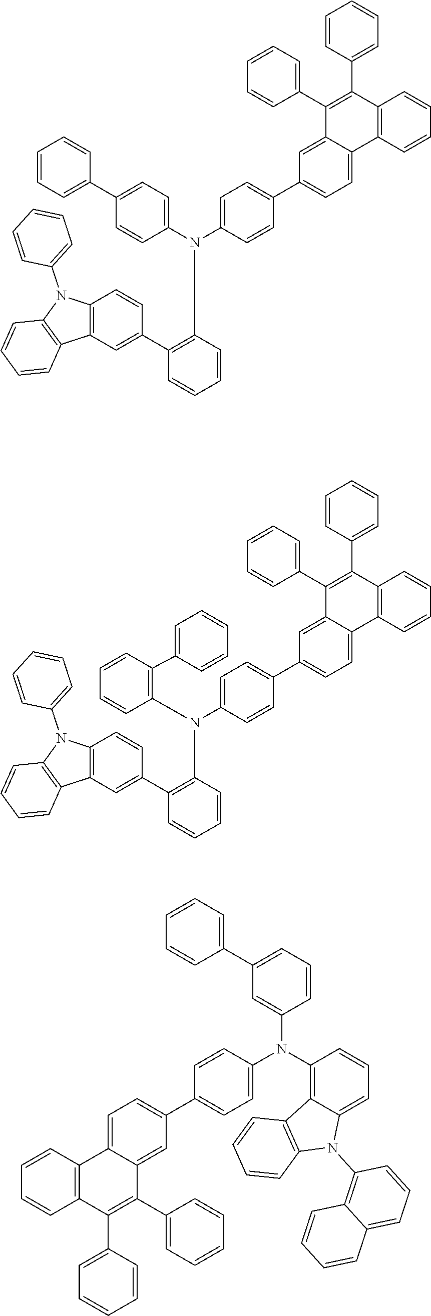 [SMILES: c1ccc(-c2ccc(N(c3ccc(-c4ccc5c(c4)c(-c4ccccc4)c(-c4ccccc4)c4ccccc45)cc3)c3ccccc3-c3ccc4c(c3)c3ccccc3n4-c3ccccc3)cc2)cc1 ; c1ccc(-c2ccccc2N(c2ccc(-c3ccc4c(c3)c(-c3ccccc3)c(-c3ccccc3)c3ccccc34)cc2)c2ccccc2-c2ccc3c(c2)c2ccccc2n3-c2ccccc2)cc1 ; c1ccc(-c2cccc(N(c3ccc(-c4ccc5c(c4)c(-c4ccccc4)c(-c4ccccc4)c4ccccc45)cc3)c3cccc4c3c3ccccc3n4-c3cccc4ccccc34)c2)cc1]

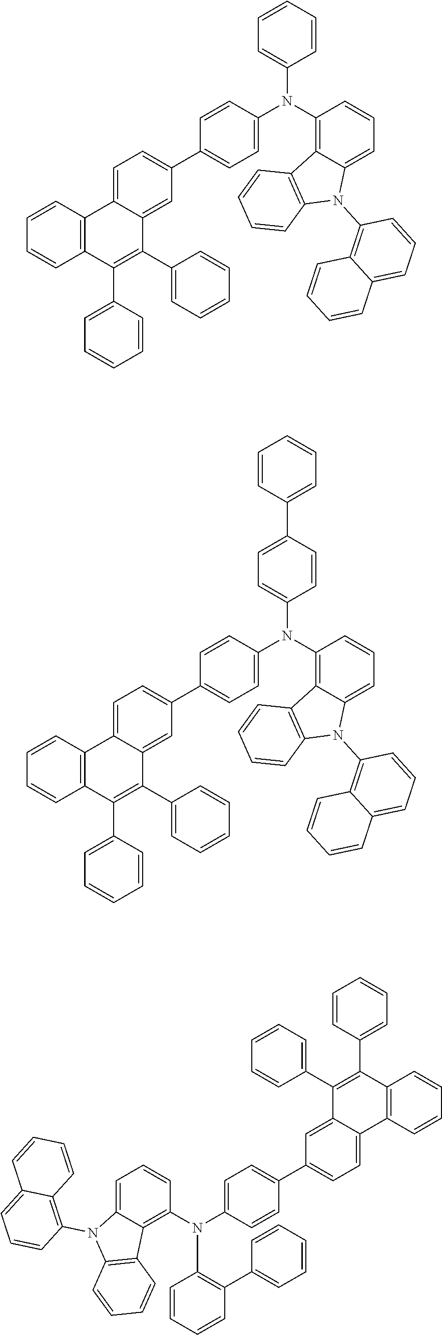 [SMILES: c1ccc(-c2c(-c3ccccc3)c3cc(-c4ccc(N(c5ccccc5)c5cccc6c5c5ccccc5n6-c5cccc6ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4c3c3ccccc3n4-c3cccc4ccccc34)cc2)cc1 ; c1ccc(-c2ccccc2N(c2ccc(-c3ccc4c(c3)c(-c3ccccc3)c(-c3ccccc3)c3ccccc34)cc2)c2cccc3c2c2ccccc2n3-c2cccc3ccccc23)cc1]

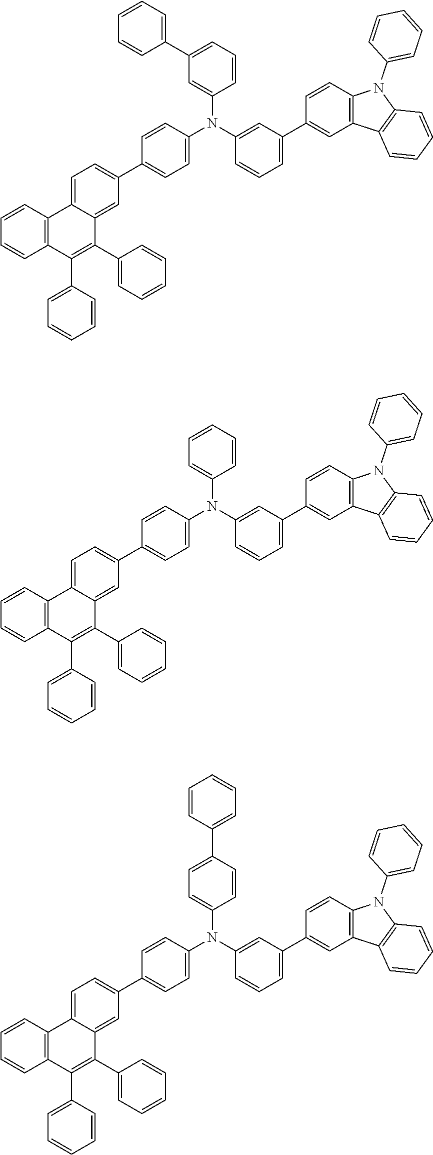 [SMILES: c1ccc(-c2cccc(N(c3ccc(-c4ccc5c(c4)c(-c4ccccc4)c(-c4ccccc4)c4ccccc45)cc3)c3cccc(-c4ccc5c(c4)c4ccccc4n5-c4ccccc4)c3)c2)cc1 ; c1ccc(-c2c(-c3ccccc3)c3cc(-c4ccc(N(c5ccccc5)c5cccc(-c6ccc7c(c6)c6ccccc6n7-c6ccccc6)c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc(-c4ccc5c(c4)c4ccccc4n5-c4ccccc4)c3)cc2)cc1]

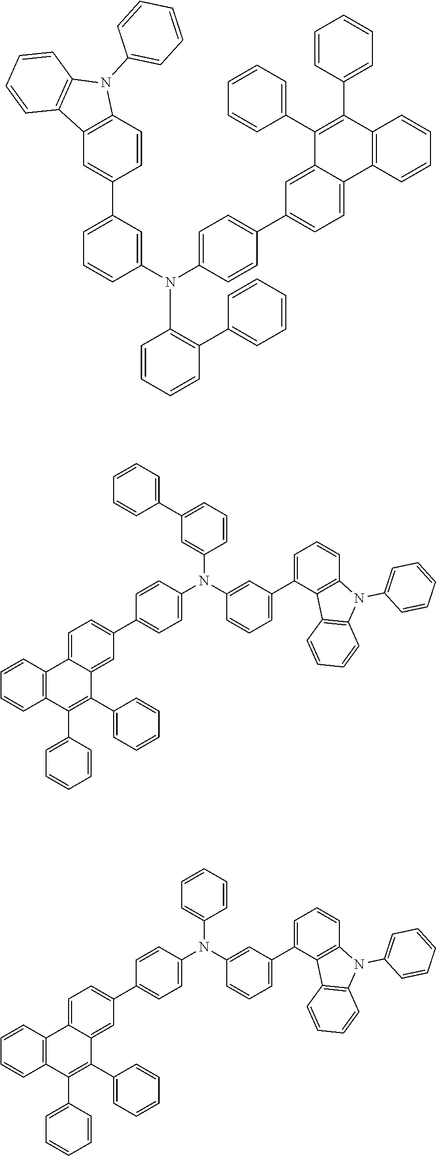 [SMILES: c1ccc(-c2ccccc2N(c2ccc(-c3ccc4c(c3)c(-c3ccccc3)c(-c3ccccc3)c3ccccc34)cc2)c2cccc(-c3ccc4c(c3)c3ccccc3n4-c3ccccc3)c2)cc1 ; c1ccc(-c2cccc(N(c3ccc(-c4ccc5c(c4)c(-c4ccccc4)c(-c4ccccc4)c4ccccc45)cc3)c3cccc(-c4cccc5c4c4ccccc4n5-c4ccccc4)c3)c2)cc1 ; c1ccc(-c2c(-c3ccccc3)c3cc(-c4ccc(N(c5ccccc5)c5cccc(-c6cccc7c6c6ccccc6n7-c6ccccc6)c5)cc4)ccc3c3ccccc23)cc1]

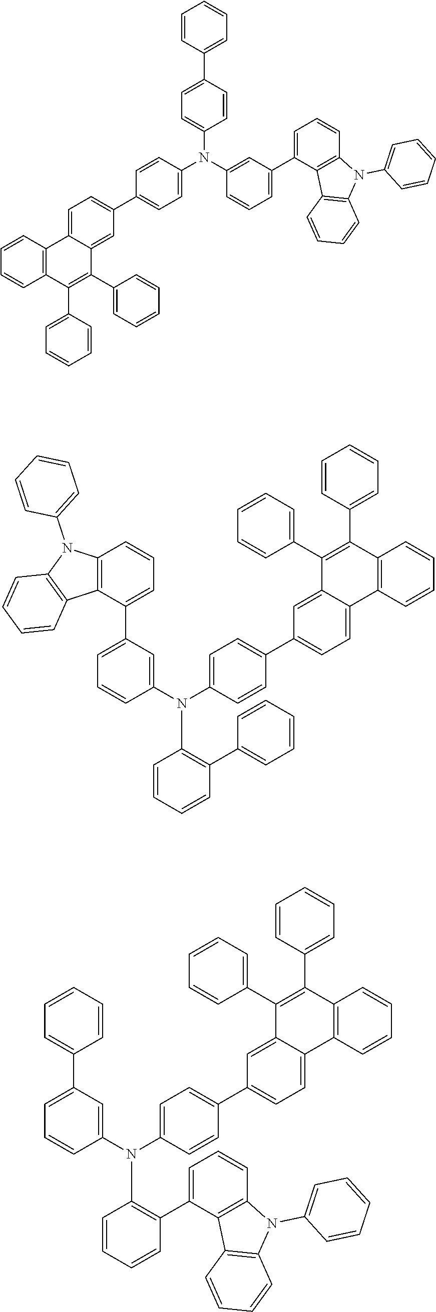 [SMILES: c1ccc(-c2ccc(N(c3ccc(-c4ccc5c(c4)c(-c4ccccc4)c(-c4ccccc4)c4ccccc45)cc3)c3cccc(-c4cccc5c4c4ccccc4n5-c4ccccc4)c3)cc2)cc1 ; c1ccc(-c2ccccc2N(c2ccc(-c3ccc4c(c3)c(-c3ccccc3)c(-c3ccccc3)c3ccccc34)cc2)c2cccc(-c3cccc4c3c3ccccc3n4-c3ccccc3)c2)cc1 ; c1ccc(-c2cccc(N(c3ccc(-c4ccc5c(c4)c(-c4ccccc4)c(-c4ccccc4)c4ccccc45)cc3)c3ccccc3-c3cccc4c3c3ccccc3n4-c3ccccc3)c2)cc1]

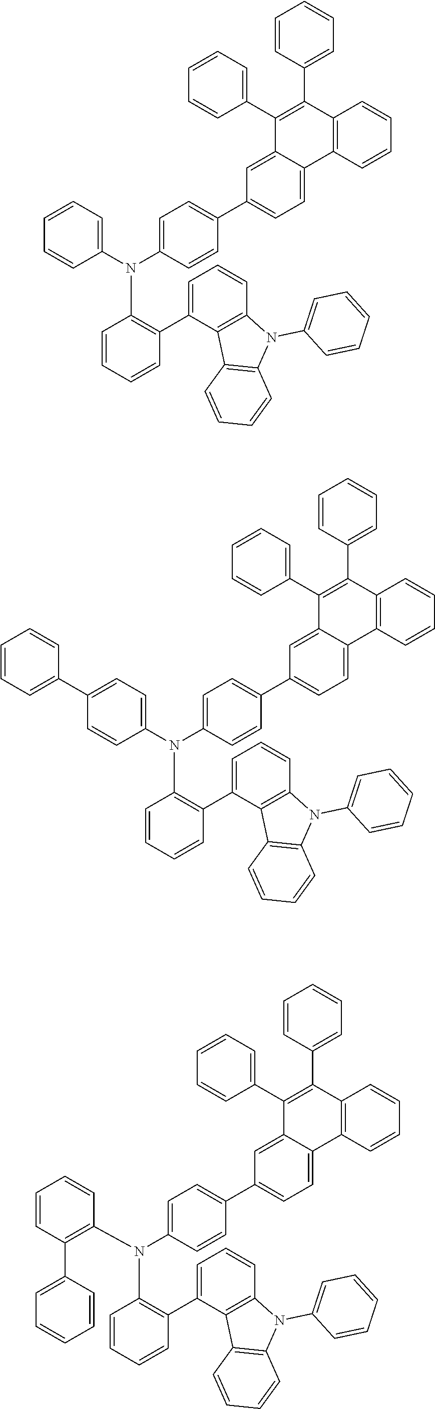 [SMILES: c1ccc(-c2c(-c3ccccc3)c3cc(-c4ccc(N(c5ccccc5)c5ccccc5-c5cccc6c5c5ccccc5n6-c5ccccc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccccc3-c3cccc4c3c3ccccc3n4-c3ccccc3)cc2)cc1 ; c1ccc(-c2ccccc2N(c2ccc(-c3ccc4c(c3)c(-c3ccccc3)c(-c3ccccc3)c3ccccc34)cc2)c2ccccc2-c2cccc3c2c2ccccc2n3-c2ccccc2)cc1]

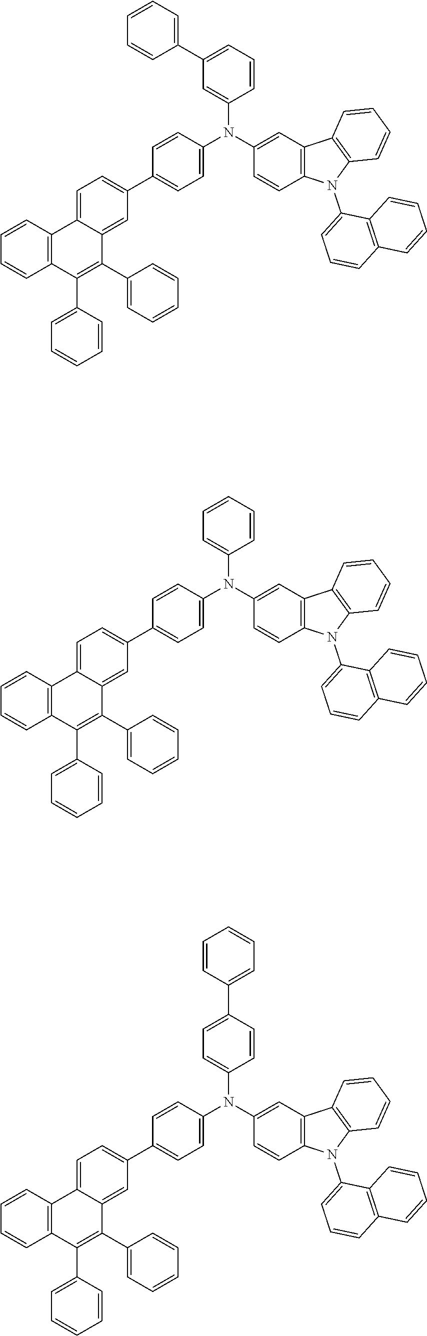 [SMILES: c1ccc(-c2cccc(N(c3ccc(-c4ccc5c(c4)c(-c4ccccc4)c(-c4ccccc4)c4ccccc45)cc3)c3ccc4c(c3)c3ccccc3n4-c3cccc4ccccc34)c2)cc1 ; c1ccc(-c2c(-c3ccccc3)c3cc(-c4ccc(N(c5ccccc5)c5ccc6c(c5)c5ccccc5n6-c5cccc6ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c(c3)c3ccccc3n4-c3cccc4ccccc34)cc2)cc1]

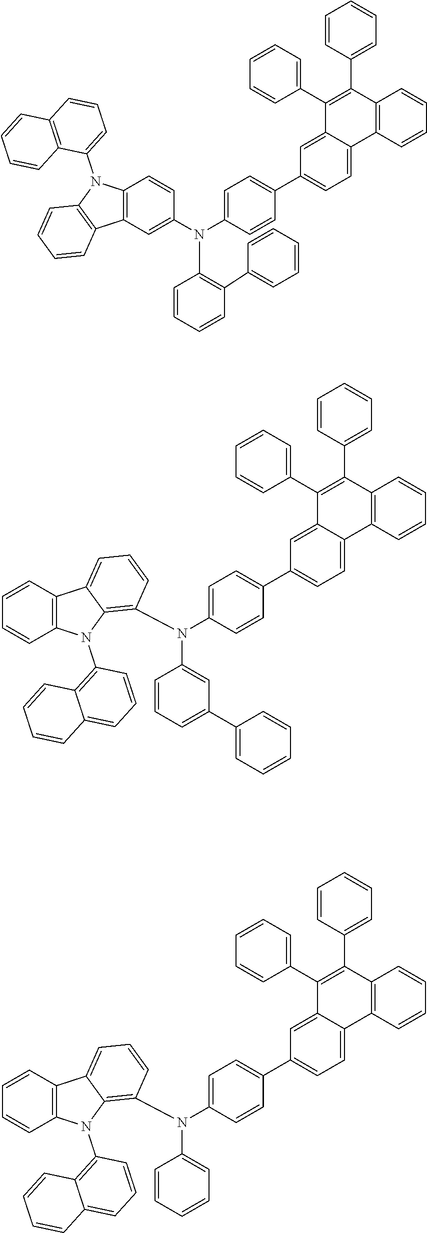 [SMILES: c1ccc(-c2ccccc2N(c2ccc(-c3ccc4c(c3)c(-c3ccccc3)c(-c3ccccc3)c3ccccc34)cc2)c2ccc3c(c2)c2ccccc2n3-c2cccc3ccccc23)cc1 ; c1ccc(-c2cccc(N(c3ccc(-c4ccc5c(c4)c(-c4ccccc4)c(-c4ccccc4)c4ccccc45)cc3)c3cccc4c5ccccc5n(-c5cccc6ccccc56)c34)c2)cc1 ; c1ccc(-c2c(-c3ccccc3)c3cc(-c4ccc(N(c5ccccc5)c5cccc6c7ccccc7n(-c7cccc8ccccc78)c56)cc4)ccc3c3ccccc23)cc1]

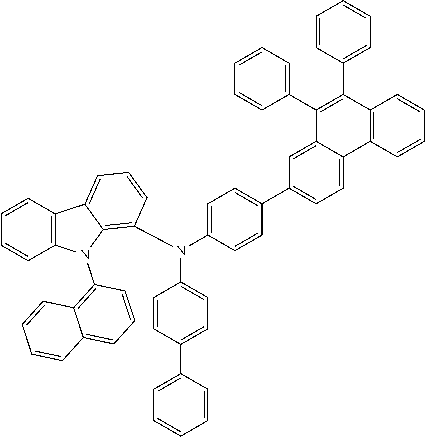 [SMILES: c1ccc(-c2ccc(N(c3ccc(-c4ccc5c(c4)c(-c4ccccc4)c(-c4ccccc4)c4ccccc45)cc3)c3cccc4c5ccccc5n(-c5cccc6ccccc56)c34)cc2)cc1]

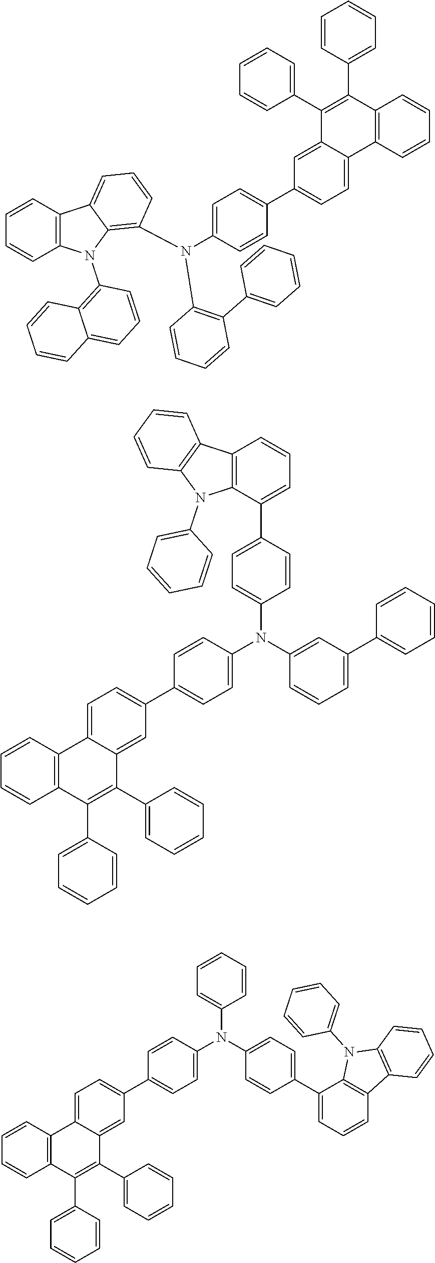 [SMILES: c1ccc(-c2ccccc2N(c2ccc(-c3ccc4c(c3)c(-c3ccccc3)c(-c3ccccc3)c3ccccc34)cc2)c2cccc3c4ccccc4n(-c4cccc5ccccc45)c23)cc1 ; c1ccc(-c2cccc(N(c3ccc(-c4ccc5c(c4)c(-c4ccccc4)c(-c4ccccc4)c4ccccc45)cc3)c3ccc(-c4cccc5c6ccccc6n(-c6ccccc6)c45)cc3)c2)cc1 ; c1ccc(-c2c(-c3ccccc3)c3cc(-c4ccc(N(c5ccccc5)c5ccc(-c6cccc7c8ccccc8n(-c8ccccc8)c67)cc5)cc4)ccc3c3ccccc23)cc1]

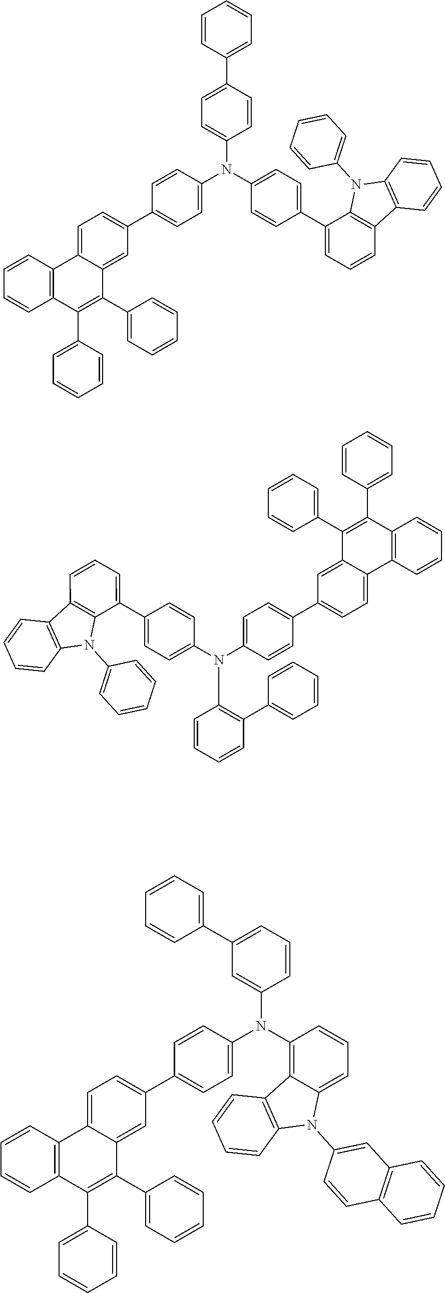 [SMILES: c1ccc(-c2ccc(N(c3ccc(-c4ccc5c(c4)c(-c4ccccc4)c(-c4ccccc4)c4ccccc45)cc3)c3ccc(-c4cccc5c6ccccc6n(-c6ccccc6)c45)cc3)cc2)cc1 ; c1ccc(-c2ccccc2N(c2ccc(-c3ccc4c(c3)c(-c3ccccc3)c(-c3ccccc3)c3ccccc34)cc2)c2ccc(-c3cccc4c5ccccc5n(-c5ccccc5)c34)cc2)cc1 ; c1ccc(-c2cccc(N(c3ccc(-c4ccc5c(c4)c(-c4ccccc4)c(-c4ccccc4)c4ccccc45)cc3)c3cccc4c3c3ccccc3n4-c3ccc4ccccc4c3)c2)cc1]

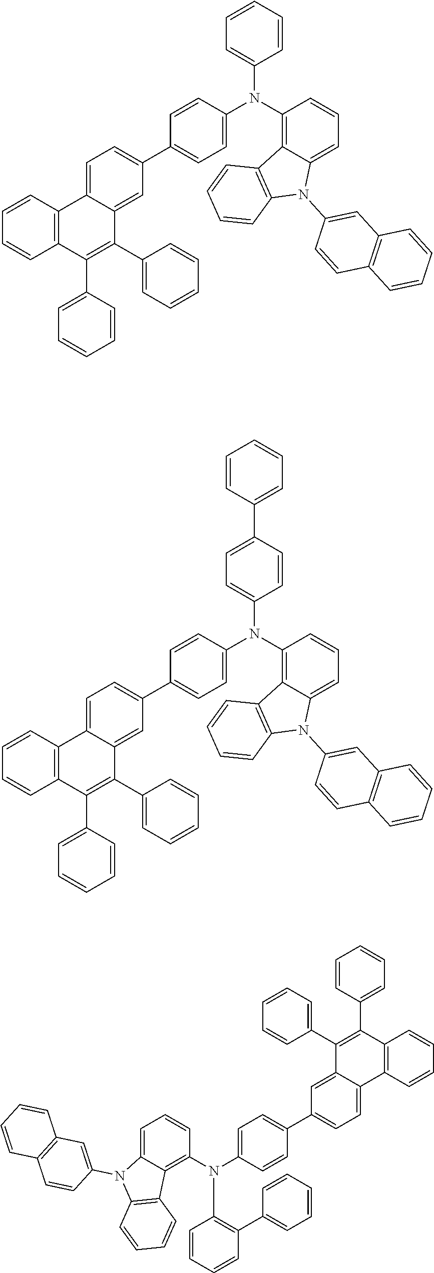 [SMILES: c1ccc(-c2c(-c3ccccc3)c3cc(-c4ccc(N(c5ccccc5)c5cccc6c5c5ccccc5n6-c5ccc6ccccc6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4c3c3ccccc3n4-c3ccc4ccccc4c3)cc2)cc1 ; c1ccc(-c2ccccc2N(c2ccc(-c3ccc4c(c3)c(-c3ccccc3)c(-c3ccccc3)c3ccccc34)cc2)c2cccc3c2c2ccccc2n3-c2ccc3ccccc3c2)cc1]

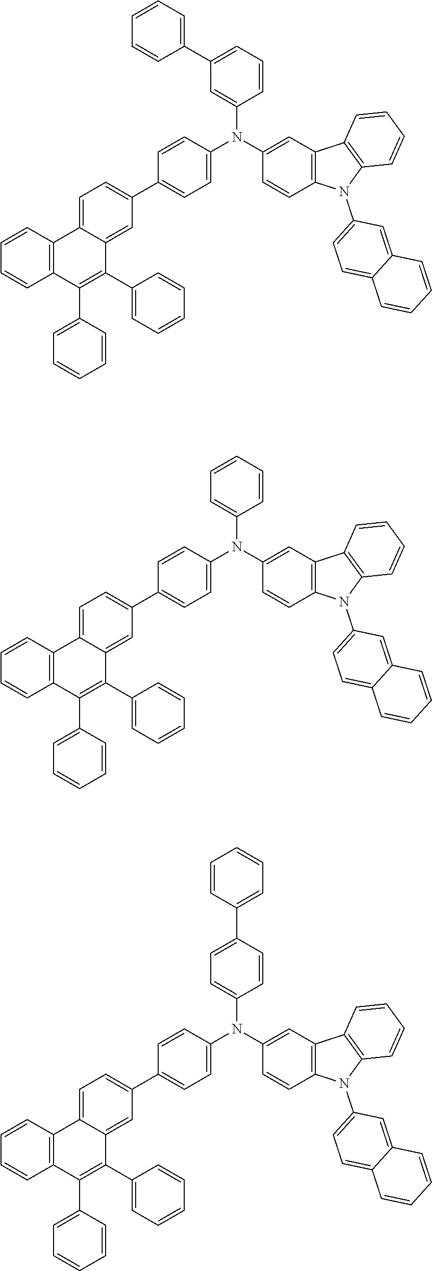 [SMILES: c1ccc(-c2cccc(N(c3ccc(-c4ccc5c(c4)c(-c4ccccc4)c(-c4ccccc4)c4ccccc45)cc3)c3ccc4c(c3)c3ccccc3n4-c3ccc4ccccc4c3)c2)cc1 ; c1ccc(-c2c(-c3ccccc3)c3cc(-c4ccc(N(c5ccccc5)c5ccc6c(c5)c5ccccc5n6-c5ccc6ccccc6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c(c3)c3ccccc3n4-c3ccc4ccccc4c3)cc2)cc1]

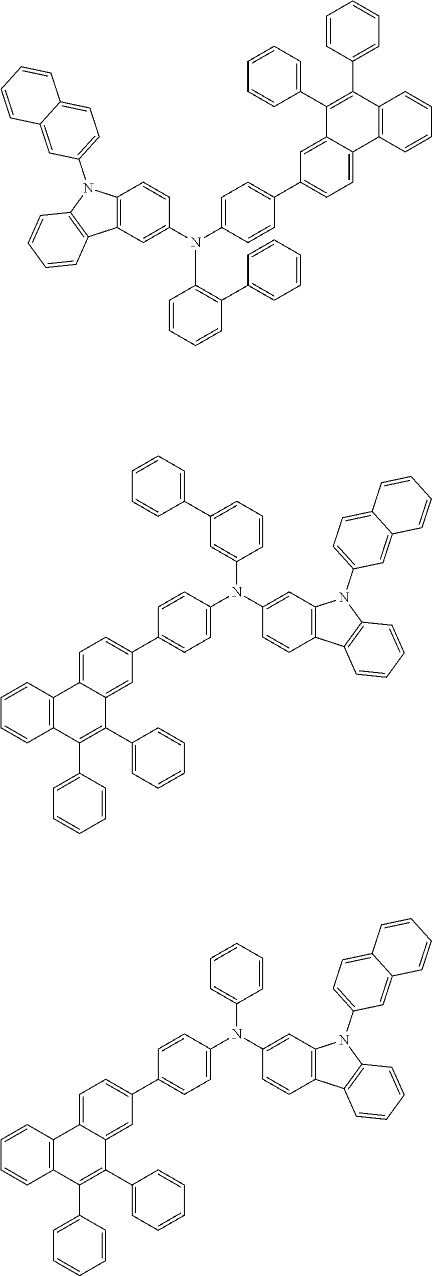 [SMILES: c1ccc(-c2ccccc2N(c2ccc(-c3ccc4c(c3)c(-c3ccccc3)c(-c3ccccc3)c3ccccc34)cc2)c2ccc3c(c2)c2ccccc2n3-c2ccc3ccccc3c2)cc1 ; c1ccc(-c2cccc(N(c3ccc(-c4ccc5c(c4)c(-c4ccccc4)c(-c4ccccc4)c4ccccc45)cc3)c3ccc4c5ccccc5n(-c5ccc6ccccc6c5)c4c3)c2)cc1 ; c1ccc(-c2c(-c3ccccc3)c3cc(-c4ccc(N(c5ccccc5)c5ccc6c7ccccc7n(-c7ccc8ccccc8c7)c6c5)cc4)ccc3c3ccccc23)cc1]

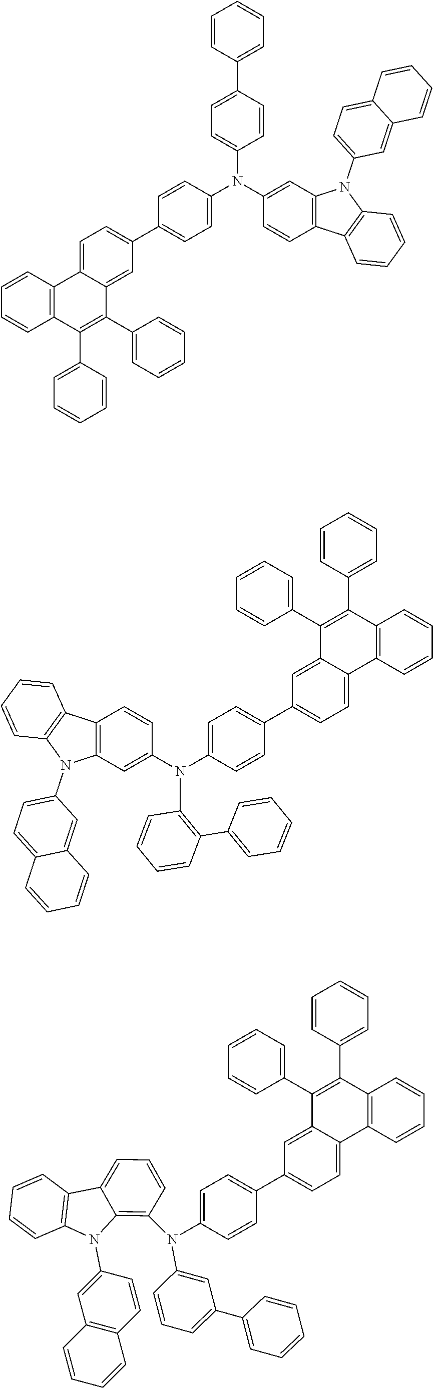 [SMILES: c1ccc(-c2ccc(N(c3ccc(-c4ccc5c(c4)c(-c4ccccc4)c(-c4ccccc4)c4ccccc45)cc3)c3ccc4c5ccccc5n(-c5ccc6ccccc6c5)c4c3)cc2)cc1 ; c1ccc(-c2ccccc2N(c2ccc(-c3ccc4c(c3)c(-c3ccccc3)c(-c3ccccc3)c3ccccc34)cc2)c2ccc3c4ccccc4n(-c4ccc5ccccc5c4)c3c2)cc1 ; c1ccc(-c2cccc(N(c3ccc(-c4ccc5c(c4)c(-c4ccccc4)c(-c4ccccc4)c4ccccc45)cc3)c3cccc4c5ccccc5n(-c5ccc6ccccc6c5)c34)c2)cc1]

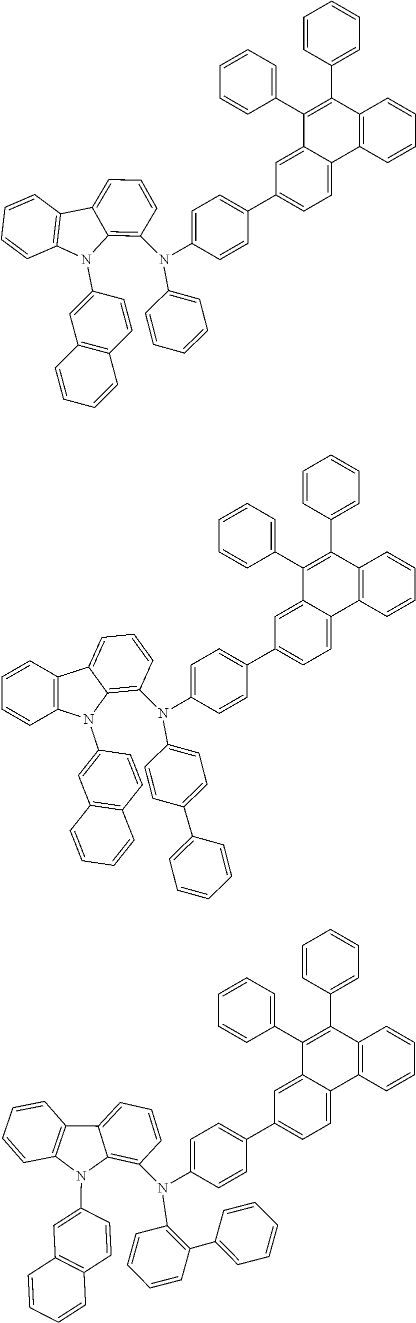 [SMILES: c1ccc(-c2c(-c3ccccc3)c3cc(-c4ccc(N(c5ccccc5)c5cccc6c7ccccc7n(-c7ccc8ccccc8c7)c56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4c5ccccc5n(-c5ccc6ccccc6c5)c34)cc2)cc1 ; c1ccc(-c2ccccc2N(c2ccc(-c3ccc4c(c3)c(-c3ccccc3)c(-c3ccccc3)c3ccccc34)cc2)c2cccc3c4ccccc4n(-c4ccc5ccccc5c4)c23)cc1]

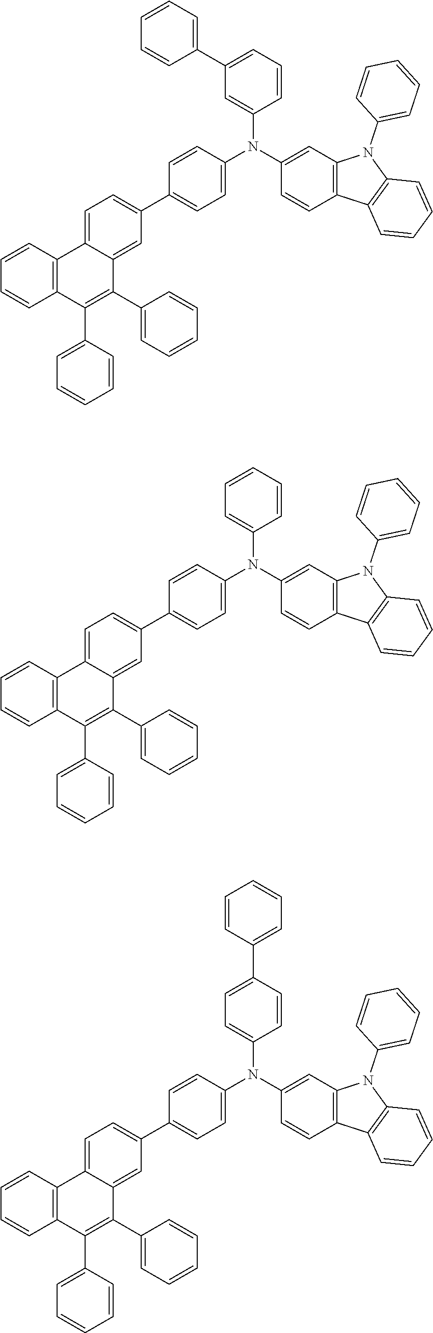 [SMILES: c1ccc(-c2cccc(N(c3ccc(-c4ccc5c(c4)c(-c4ccccc4)c(-c4ccccc4)c4ccccc45)cc3)c3ccc4c5ccccc5n(-c5ccccc5)c4c3)c2)cc1 ; c1ccc(-c2c(-c3ccccc3)c3cc(-c4ccc(N(c5ccccc5)c5ccc6c7ccccc7n(-c7ccccc7)c6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c5ccccc5n(-c5ccccc5)c4c3)cc2)cc1]

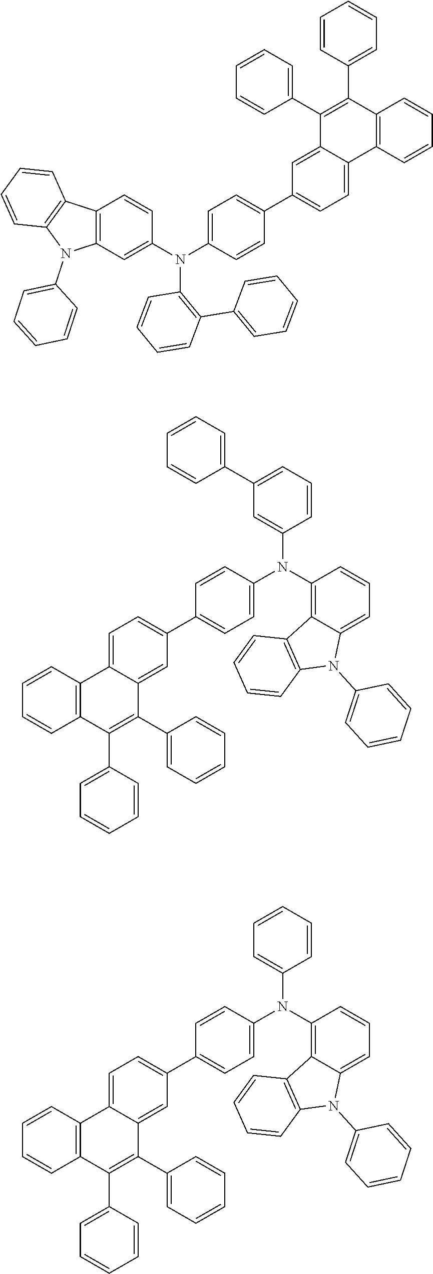 [SMILES: c1ccc(-c2ccccc2N(c2ccc(-c3ccc4c(c3)c(-c3ccccc3)c(-c3ccccc3)c3ccccc34)cc2)c2ccc3c4ccccc4n(-c4ccccc4)c3c2)cc1 ; c1ccc(-c2cccc(N(c3ccc(-c4ccc5c(c4)c(-c4ccccc4)c(-c4ccccc4)c4ccccc45)cc3)c3cccc4c3c3ccccc3n4-c3ccccc3)c2)cc1 ; c1ccc(-c2c(-c3ccccc3)c3cc(-c4ccc(N(c5ccccc5)c5cccc6c5c5ccccc5n6-c5ccccc5)cc4)ccc3c3ccccc23)cc1]

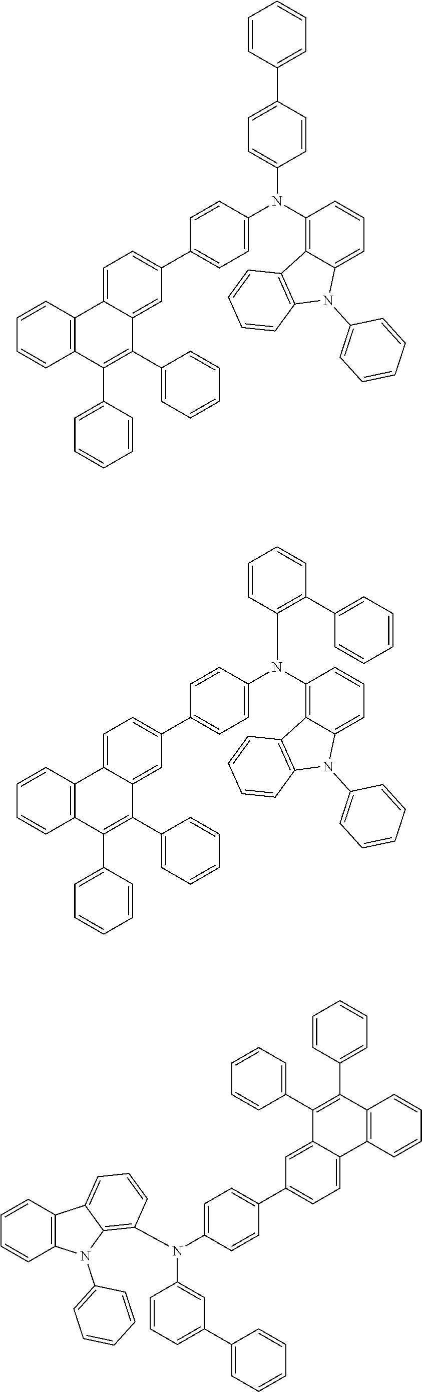 [SMILES: c1ccc(-c2ccc(N(c3ccc(-c4ccc5c(c4)c(-c4ccccc4)c(-c4ccccc4)c4ccccc45)cc3)c3cccc4c3c3ccccc3n4-c3ccccc3)cc2)cc1 ; c1ccc(-c2ccccc2N(c2ccc(-c3ccc4c(c3)c(-c3ccccc3)c(-c3ccccc3)c3ccccc34)cc2)c2cccc3c2c2ccccc2n3-c2ccccc2)cc1 ; c1ccc(-c2cccc(N(c3ccc(-c4ccc5c(c4)c(-c4ccccc4)c(-c4ccccc4)c4ccccc45)cc3)c3cccc4c5ccccc5n(-c5ccccc5)c34)c2)cc1]

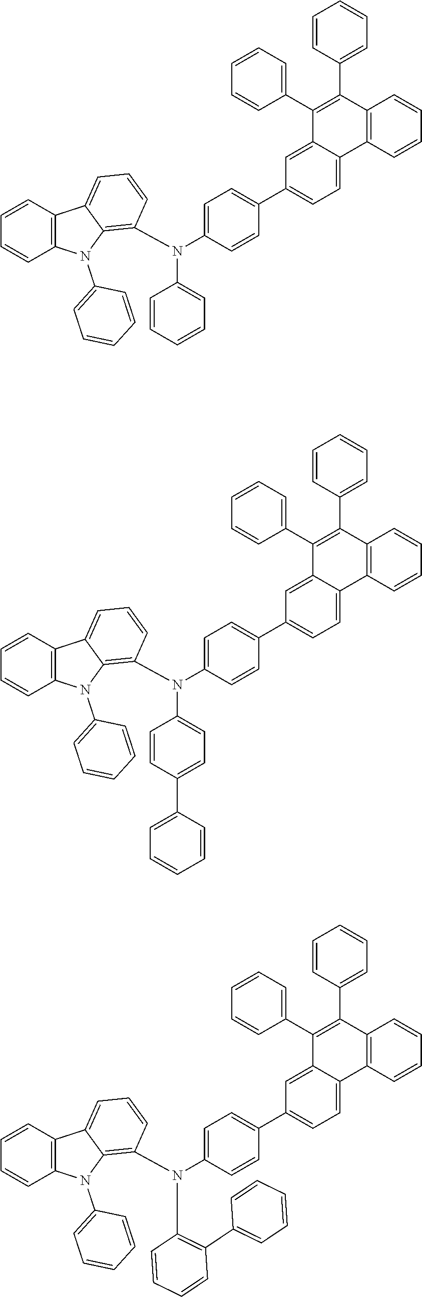 [SMILES: c1ccc(-c2c(-c3ccccc3)c3cc(-c4ccc(N(c5ccccc5)c5cccc6c7ccccc7n(-c7ccccc7)c56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4c5ccccc5n(-c5ccccc5)c34)cc2)cc1 ; c1ccc(-c2ccccc2N(c2ccc(-c3ccc4c(c3)c(-c3ccccc3)c(-c3ccccc3)c3ccccc34)cc2)c2cccc3c4ccccc4n(-c4ccccc4)c23)cc1]

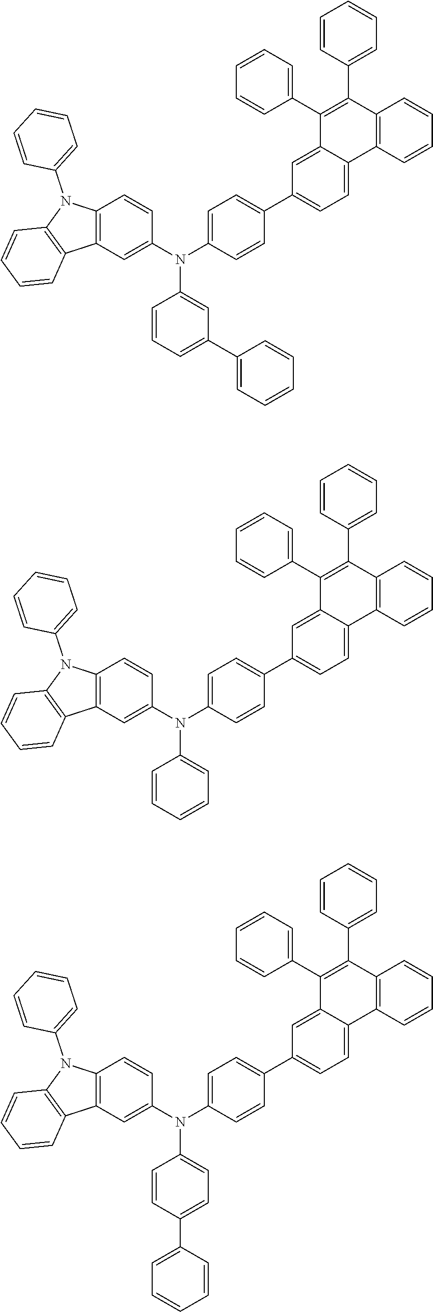 [SMILES: c1ccc(-c2cccc(N(c3ccc(-c4ccc5c(c4)c(-c4ccccc4)c(-c4ccccc4)c4ccccc45)cc3)c3ccc4c(c3)c3ccccc3n4-c3ccccc3)c2)cc1 ; c1ccc(-c2c(-c3ccccc3)c3cc(-c4ccc(N(c5ccccc5)c5ccc6c(c5)c5ccccc5n6-c5ccccc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4c(c3)c3ccccc3n4-c3ccccc3)cc2)cc1]

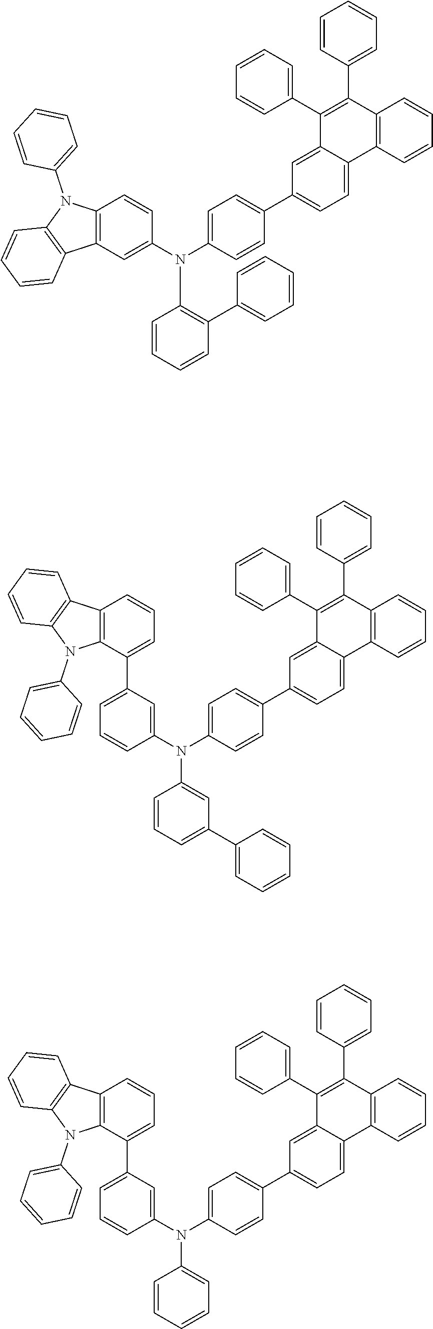 [SMILES: c1ccc(-c2ccccc2N(c2ccc(-c3ccc4c(c3)c(-c3ccccc3)c(-c3ccccc3)c3ccccc34)cc2)c2ccc3c(c2)c2ccccc2n3-c2ccccc2)cc1 ; c1ccc(-c2cccc(N(c3ccc(-c4ccc5c(c4)c(-c4ccccc4)c(-c4ccccc4)c4ccccc45)cc3)c3cccc(-c4cccc5c6ccccc6n(-c6ccccc6)c45)c3)c2)cc1 ; c1ccc(-c2c(-c3ccccc3)c3cc(-c4ccc(N(c5ccccc5)c5cccc(-c6cccc7c8ccccc8n(-c8ccccc8)c67)c5)cc4)ccc3c3ccccc23)cc1]

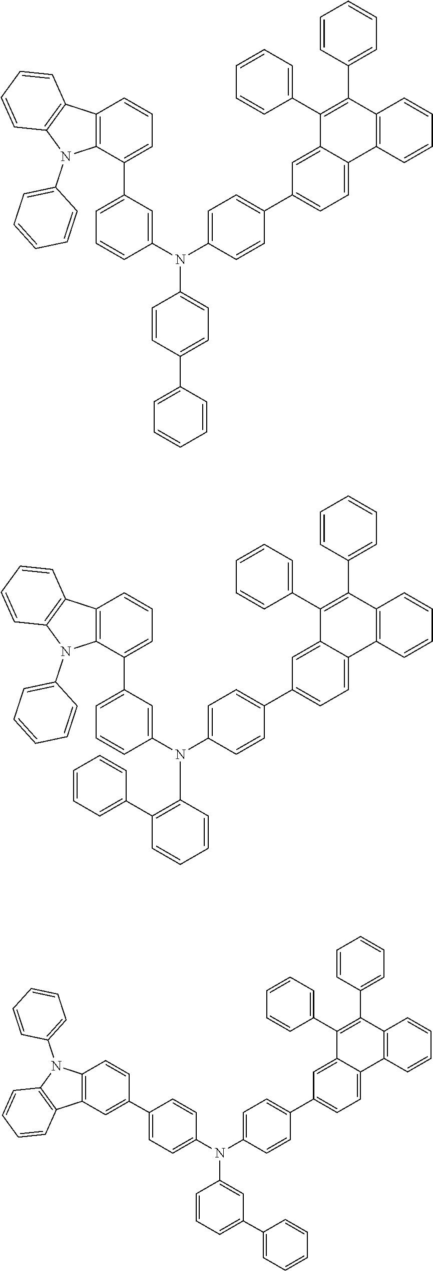 [SMILES: c1ccc(-c2ccc(N(c3ccc(-c4ccc5c(c4)c(-c4ccccc4)c(-c4ccccc4)c4ccccc45)cc3)c3cccc(-c4cccc5c6ccccc6n(-c6ccccc6)c45)c3)cc2)cc1 ; c1ccc(-c2ccccc2N(c2ccc(-c3ccc4c(c3)c(-c3ccccc3)c(-c3ccccc3)c3ccccc34)cc2)c2cccc(-c3cccc4c5ccccc5n(-c5ccccc5)c34)c2)cc1 ; c1ccc(-c2cccc(N(c3ccc(-c4ccc5c(c4)c(-c4ccccc4)c(-c4ccccc4)c4ccccc45)cc3)c3ccc(-c4ccc5c(c4)c4ccccc4n5-c4ccccc4)cc3)c2)cc1]

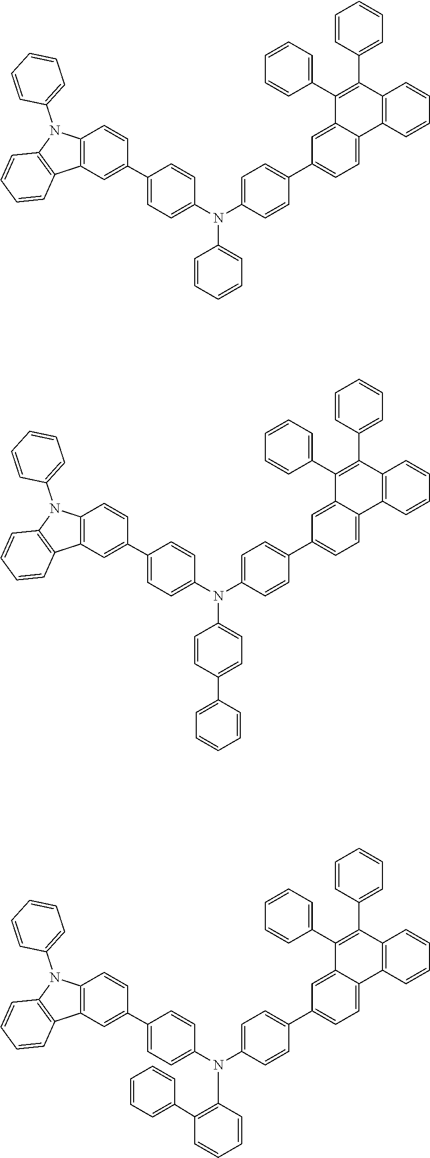 [SMILES: c1ccc(-c2c(-c3ccccc3)c3cc(-c4ccc(N(c5ccccc5)c5ccc(-c6ccc7c(c6)c6ccccc6n7-c6ccccc6)cc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc(-c4ccc5c(c4)c4ccccc4n5-c4ccccc4)cc3)cc2)cc1 ; c1ccc(-c2ccccc2N(c2ccc(-c3ccc4c(c3)c(-c3ccccc3)c(-c3ccccc3)c3ccccc34)cc2)c2ccc(-c3ccc4c(c3)c3ccccc3n4-c3ccccc3)cc2)cc1]

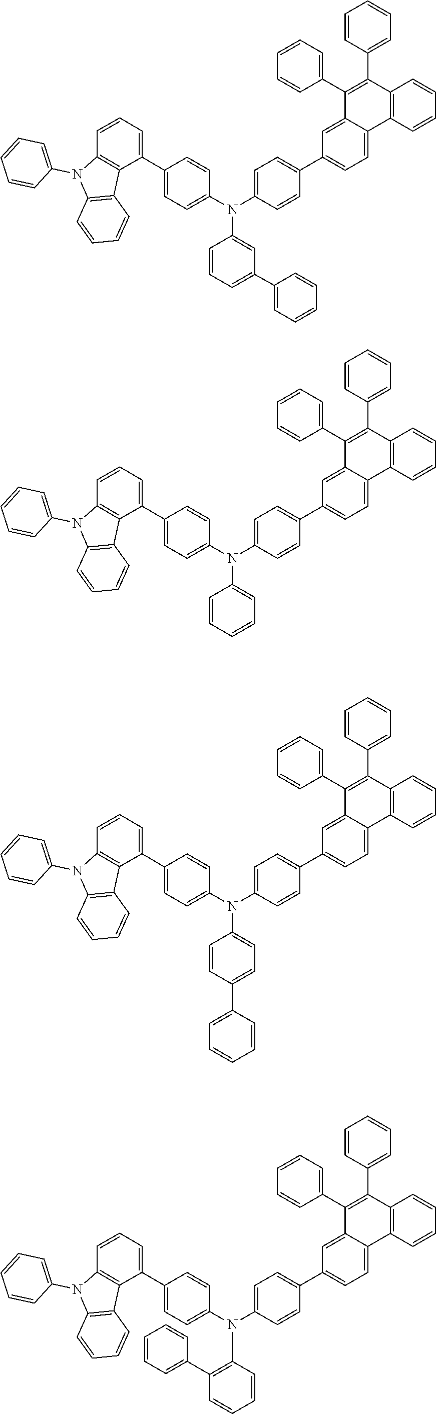 [SMILES: c1ccc(-c2cccc(N(c3ccc(-c4ccc5c(c4)c(-c4ccccc4)c(-c4ccccc4)c4ccccc45)cc3)c3ccc(-c4cccc5c4c4ccccc4n5-c4ccccc4)cc3)c2)cc1 ; c1ccc(-c2c(-c3ccccc3)c3cc(-c4ccc(N(c5ccccc5)c5ccc(-c6cccc7c6c6ccccc6n7-c6ccccc6)cc5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc(-c4cccc5c4c4ccccc4n5-c4ccccc4)cc3)cc2)cc1 ; c1ccc(-c2ccccc2N(c2ccc(-c3ccc4c(c3)c(-c3ccccc3)c(-c3ccccc3)c3ccccc34)cc2)c2ccc(-c3cccc4c3c3ccccc3n4-c3ccccc3)cc2)cc1]

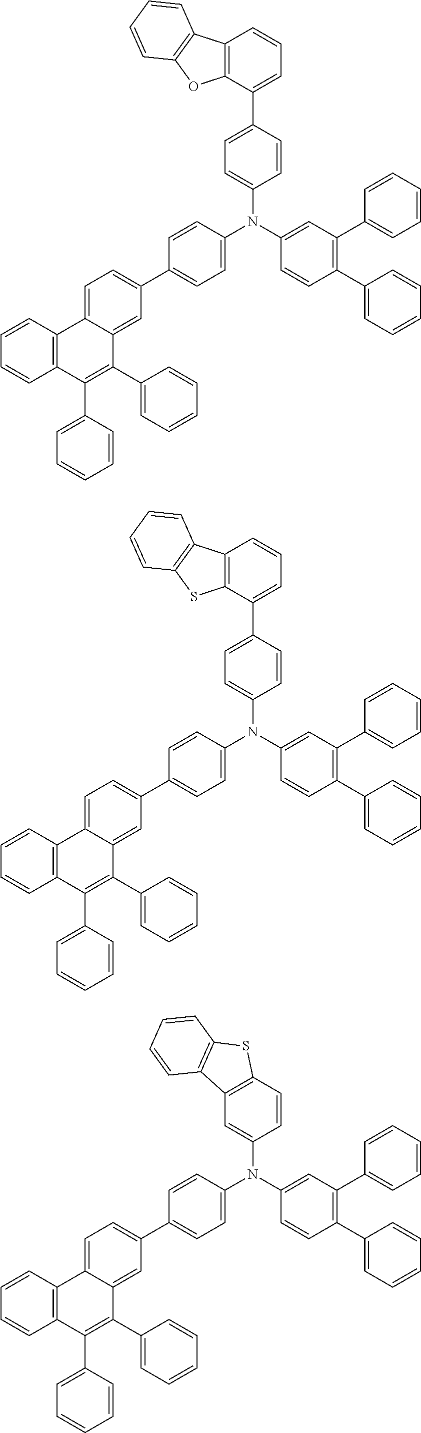 [SMILES: c1ccc(-c2ccc(N(c3ccc(-c4ccc5c(c4)c(-c4ccccc4)c(-c4ccccc4)c4ccccc45)cc3)c3ccc(-c4cccc5c4oc4ccccc45)cc3)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc(-c4cccc5c4sc4ccccc45)cc3)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc4sc5ccccc5c4c3)cc2-c2ccccc2)cc1]

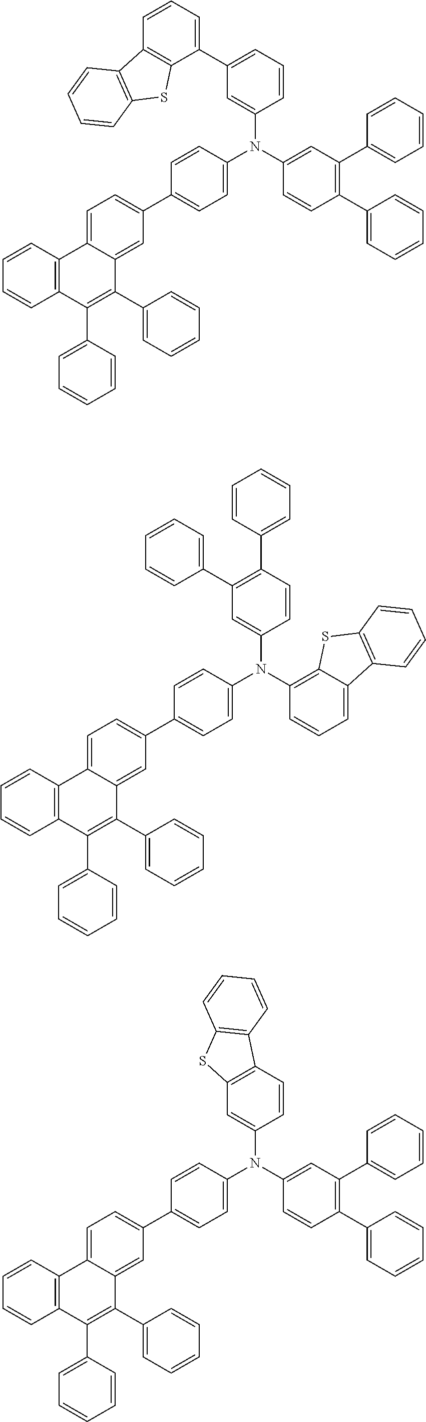 [SMILES: c1ccc(-c2ccc(N(c3ccc(-c4ccc5c(c4)c(-c4ccccc4)c(-c4ccccc4)c4ccccc45)cc3)c3cccc(-c4cccc5c4sc4ccccc45)c3)cc2-c2ccccc2)cc1 ; c1ccc(-c2ccc(N(c3ccc(-c4ccc5c(c4)c(-c4ccccc4)c(-c4ccccc4)c4ccccc45)cc3)c3cccc4c3sc3ccccc34)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc4c(c3)sc3ccccc34)cc2-c2ccccc2)cc1]

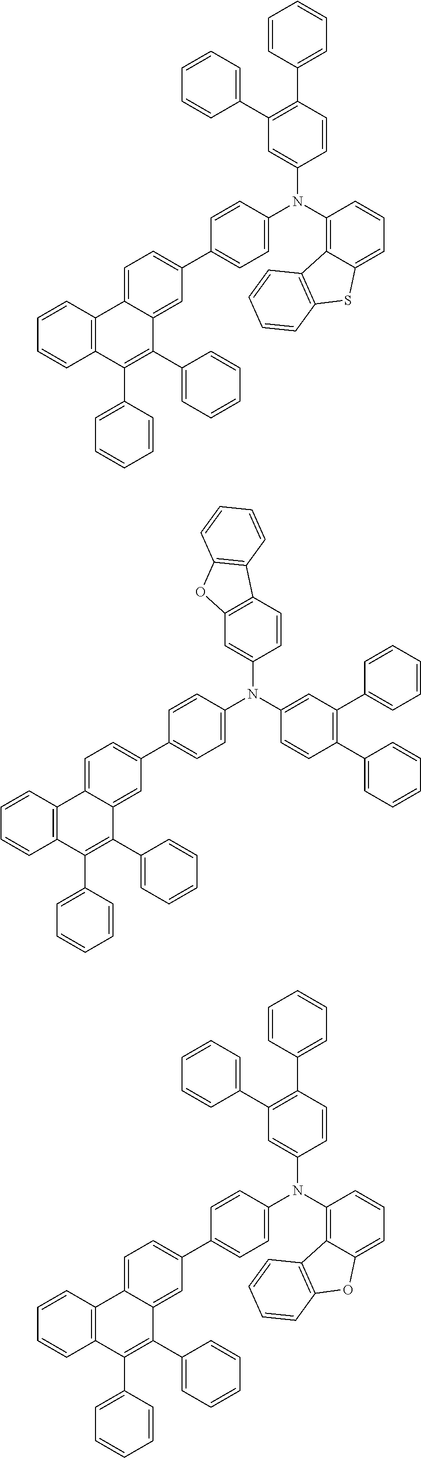 [SMILES: c1ccc(-c2ccc(N(c3ccc(-c4ccc5c(c4)c(-c4ccccc4)c(-c4ccccc4)c4ccccc45)cc3)c3cccc4sc5ccccc5c34)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc4c(c3)oc3ccccc34)cc2-c2ccccc2)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)cc2-c2ccccc2)cc1]

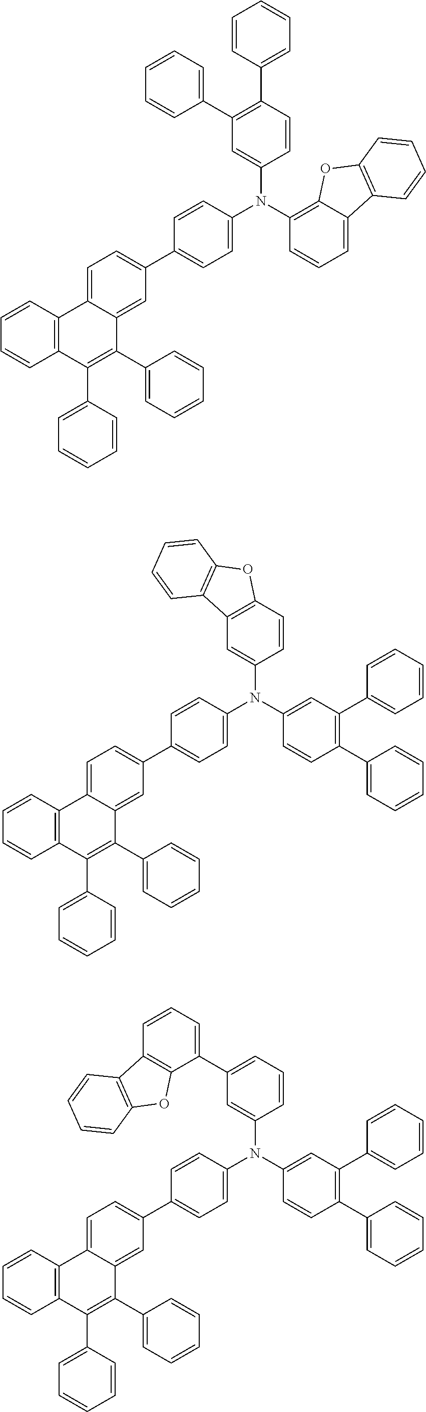 [SMILES: c1ccc(-c2ccc(N(c3ccc(-c4ccc5c(c4)c(-c4ccccc4)c(-c4ccccc4)c4ccccc45)cc3)c3cccc4c3oc3ccccc34)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc4oc5ccccc5c4c3)cc2-c2ccccc2)cc1 ; c1ccc(-c2ccc(N(c3ccc(-c4ccc5c(c4)c(-c4ccccc4)c(-c4ccccc4)c4ccccc45)cc3)c3cccc(-c4cccc5c4oc4ccccc45)c3)cc2-c2ccccc2)cc1]

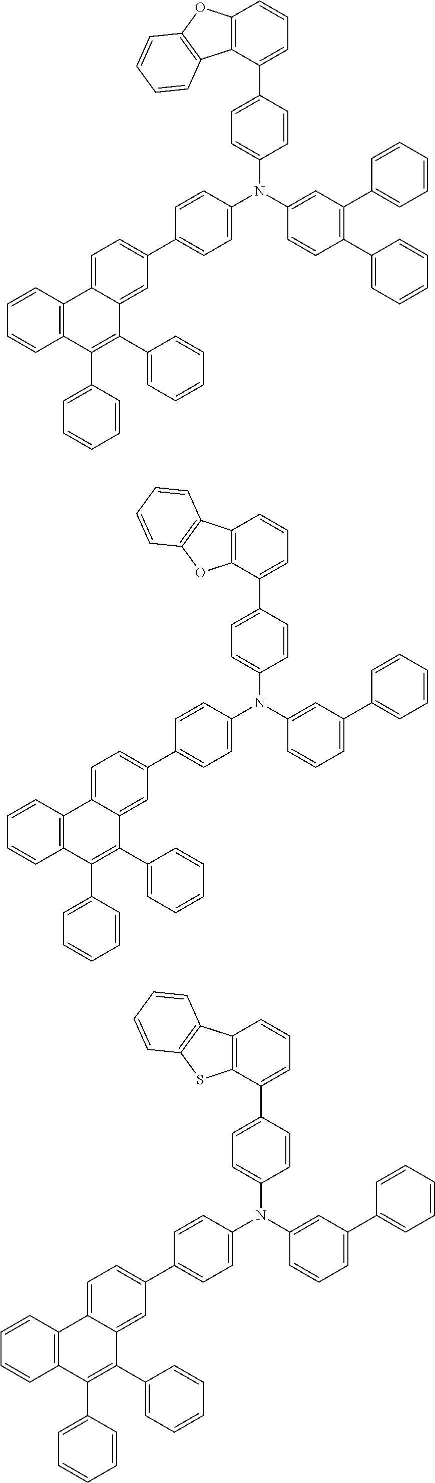 [SMILES: c1ccc(-c2ccc(N(c3ccc(-c4ccc5c(c4)c(-c4ccccc4)c(-c4ccccc4)c4ccccc45)cc3)c3ccc(-c4cccc5oc6ccccc6c45)cc3)cc2-c2ccccc2)cc1 ; c1ccc(-c2cccc(N(c3ccc(-c4ccc5c(c4)c(-c4ccccc4)c(-c4ccccc4)c4ccccc45)cc3)c3ccc(-c4cccc5c4oc4ccccc45)cc3)c2)cc1 ; c1ccc(-c2cccc(N(c3ccc(-c4ccc5c(c4)c(-c4ccccc4)c(-c4ccccc4)c4ccccc45)cc3)c3ccc(-c4cccc5c4sc4ccccc45)cc3)c2)cc1]

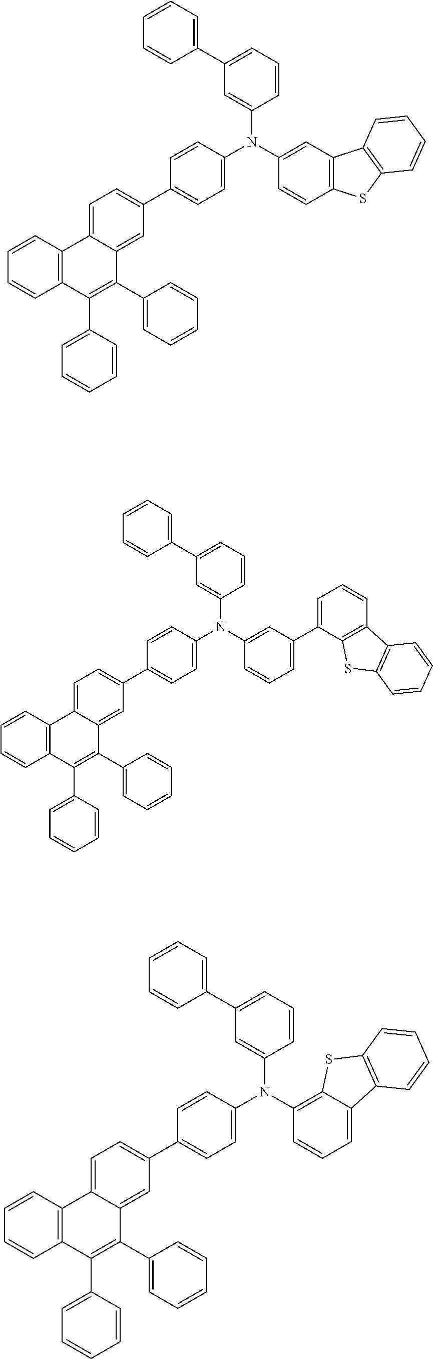 [SMILES: c1ccc(-c2cccc(N(c3ccc(-c4ccc5c(c4)c(-c4ccccc4)c(-c4ccccc4)c4ccccc45)cc3)c3ccc4sc5ccccc5c4c3)c2)cc1 ; c1ccc(-c2cccc(N(c3ccc(-c4ccc5c(c4)c(-c4ccccc4)c(-c4ccccc4)c4ccccc45)cc3)c3cccc(-c4cccc5c4sc4ccccc45)c3)c2)cc1 ; c1ccc(-c2cccc(N(c3ccc(-c4ccc5c(c4)c(-c4ccccc4)c(-c4ccccc4)c4ccccc45)cc3)c3cccc4c3sc3ccccc34)c2)cc1]

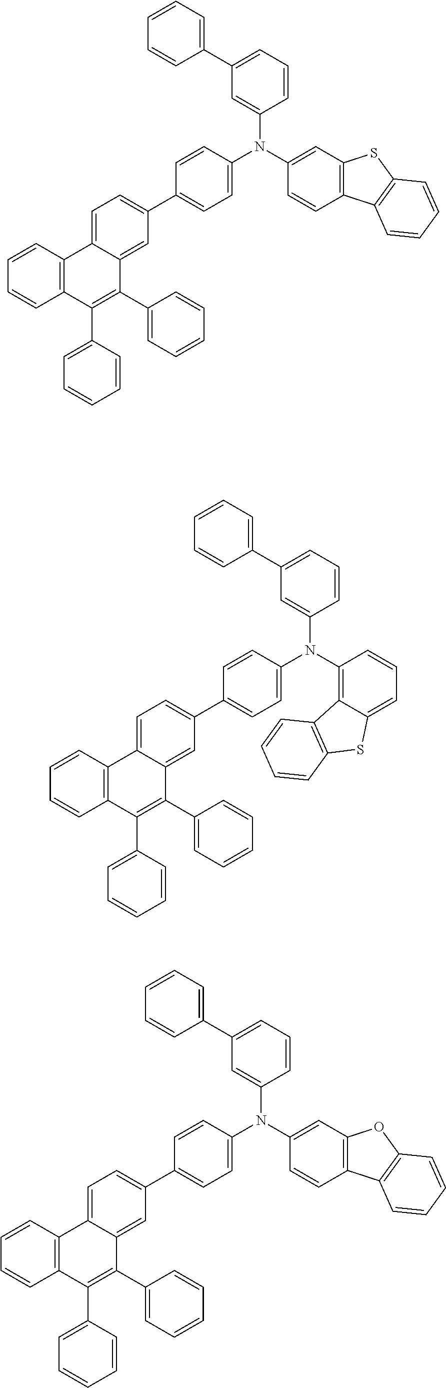 [SMILES: c1ccc(-c2cccc(N(c3ccc(-c4ccc5c(c4)c(-c4ccccc4)c(-c4ccccc4)c4ccccc45)cc3)c3ccc4c(c3)sc3ccccc34)c2)cc1 ; c1ccc(-c2cccc(N(c3ccc(-c4ccc5c(c4)c(-c4ccccc4)c(-c4ccccc4)c4ccccc45)cc3)c3cccc4sc5ccccc5c34)c2)cc1 ; c1ccc(-c2cccc(N(c3ccc(-c4ccc5c(c4)c(-c4ccccc4)c(-c4ccccc4)c4ccccc45)cc3)c3ccc4c(c3)oc3ccccc34)c2)cc1]

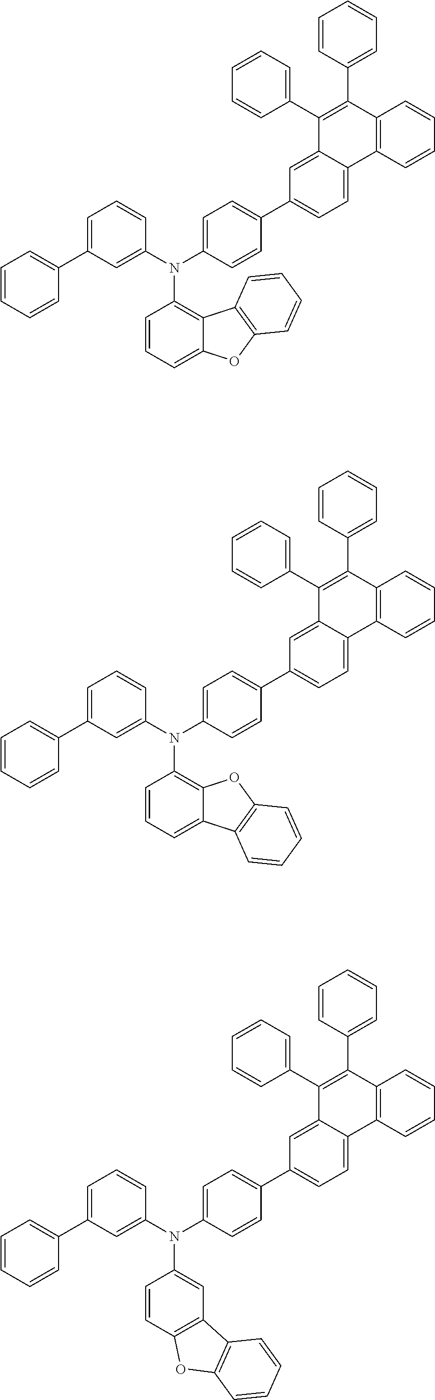 [SMILES: c1ccc(-c2cccc(N(c3ccc(-c4ccc5c(c4)c(-c4ccccc4)c(-c4ccccc4)c4ccccc45)cc3)c3cccc4oc5ccccc5c34)c2)cc1 ; c1ccc(-c2cccc(N(c3ccc(-c4ccc5c(c4)c(-c4ccccc4)c(-c4ccccc4)c4ccccc45)cc3)c3cccc4c3oc3ccccc34)c2)cc1 ; c1ccc(-c2cccc(N(c3ccc(-c4ccc5c(c4)c(-c4ccccc4)c(-c4ccccc4)c4ccccc45)cc3)c3ccc4oc5ccccc5c4c3)c2)cc1]

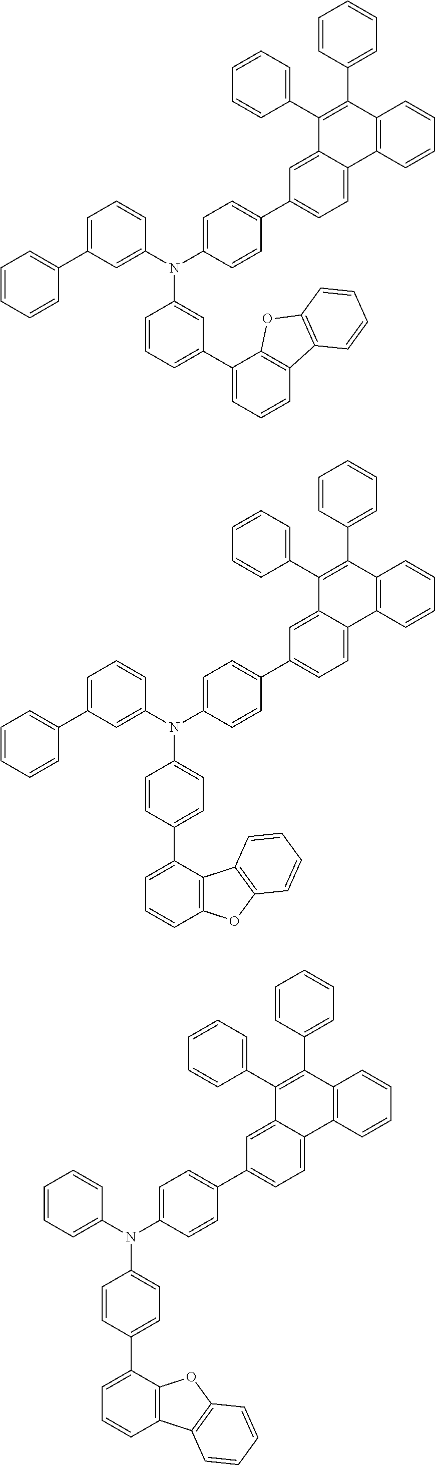 [SMILES: c1ccc(-c2cccc(N(c3ccc(-c4ccc5c(c4)c(-c4ccccc4)c(-c4ccccc4)c4ccccc45)cc3)c3cccc(-c4cccc5c4oc4ccccc45)c3)c2)cc1 ; c1ccc(-c2cccc(N(c3ccc(-c4ccc5c(c4)c(-c4ccccc4)c(-c4ccccc4)c4ccccc45)cc3)c3ccc(-c4cccc5oc6ccccc6c45)cc3)c2)cc1 ; c1ccc(-c2c(-c3ccccc3)c3cc(-c4ccc(N(c5ccccc5)c5ccc(-c6cccc7c6oc6ccccc67)cc5)cc4)ccc3c3ccccc23)cc1]

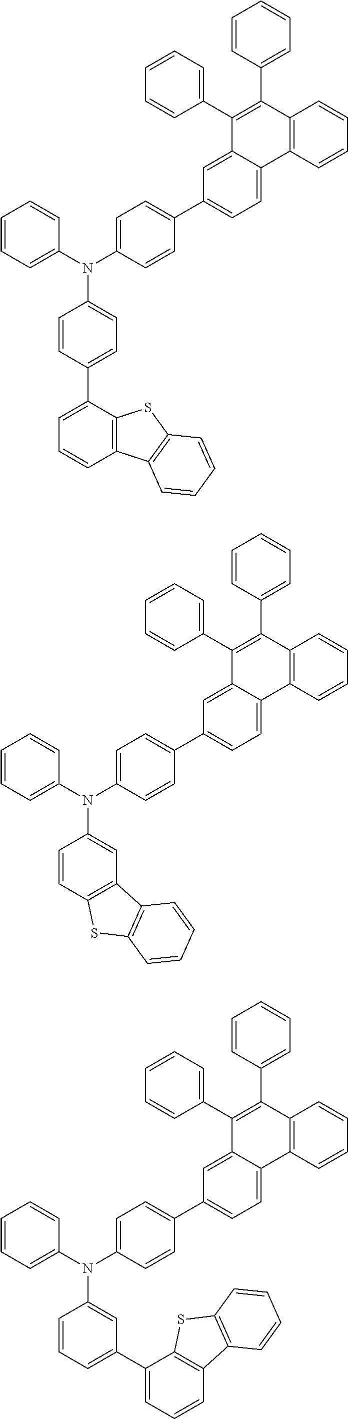 [SMILES: c1ccc(-c2c(-c3ccccc3)c3cc(-c4ccc(N(c5ccccc5)c5ccc(-c6cccc7c6sc6ccccc67)cc5)cc4)ccc3c3ccccc23)cc1 ; c1ccc(-c2c(-c3ccccc3)c3cc(-c4ccc(N(c5ccccc5)c5ccc6sc7ccccc7c6c5)cc4)ccc3c3ccccc23)cc1 ; c1ccc(-c2c(-c3ccccc3)c3cc(-c4ccc(N(c5ccccc5)c5cccc(-c6cccc7c6sc6ccccc67)c5)cc4)ccc3c3ccccc23)cc1]

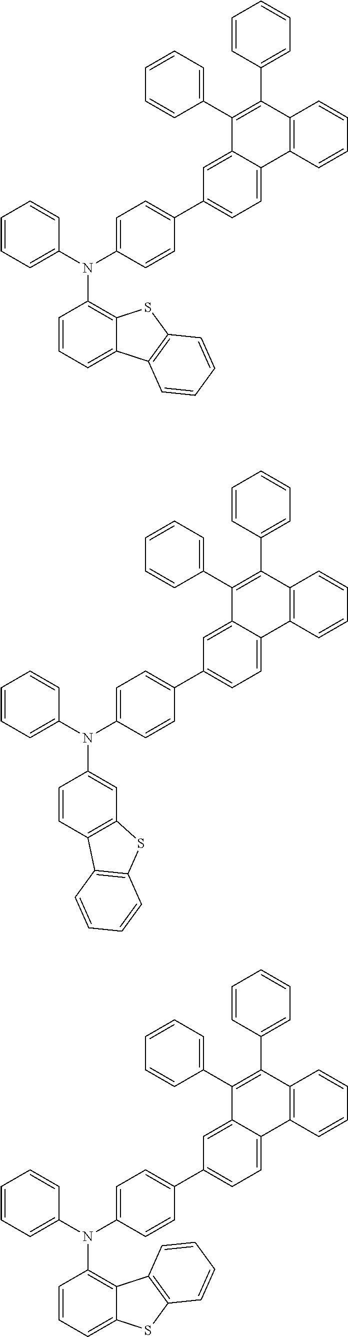 [SMILES: c1ccc(-c2c(-c3ccccc3)c3cc(-c4ccc(N(c5ccccc5)c5cccc6c5sc5ccccc56)cc4)ccc3c3ccccc23)cc1 ; c1ccc(-c2c(-c3ccccc3)c3cc(-c4ccc(N(c5ccccc5)c5ccc6c(c5)sc5ccccc56)cc4)ccc3c3ccccc23)cc1 ; c1ccc(-c2c(-c3ccccc3)c3cc(-c4ccc(N(c5ccccc5)c5cccc6sc7ccccc7c56)cc4)ccc3c3ccccc23)cc1]

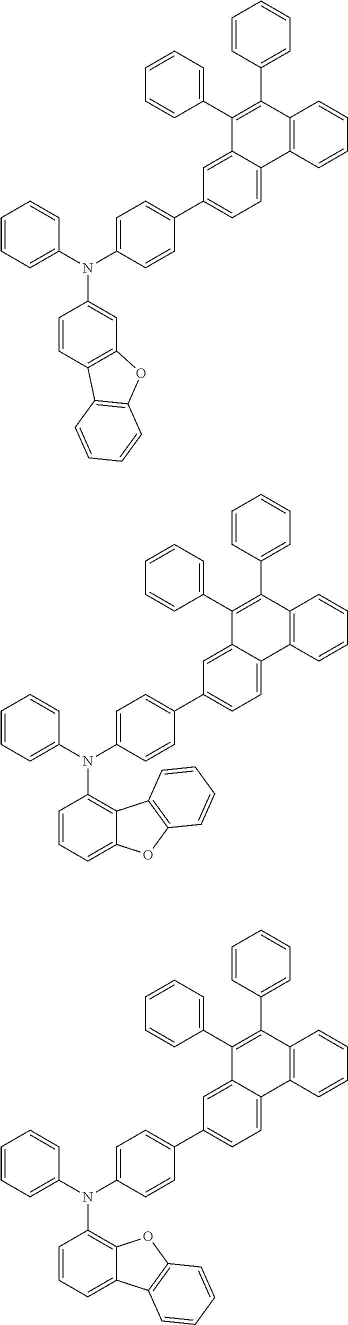 [SMILES: c1ccc(-c2c(-c3ccccc3)c3cc(-c4ccc(N(c5ccccc5)c5ccc6c(c5)oc5ccccc56)cc4)ccc3c3ccccc23)cc1 ; c1ccc(-c2c(-c3ccccc3)c3cc(-c4ccc(N(c5ccccc5)c5cccc6oc7ccccc7c56)cc4)ccc3c3ccccc23)cc1 ; c1ccc(-c2c(-c3ccccc3)c3cc(-c4ccc(N(c5ccccc5)c5cccc6c5oc5ccccc56)cc4)ccc3c3ccccc23)cc1]

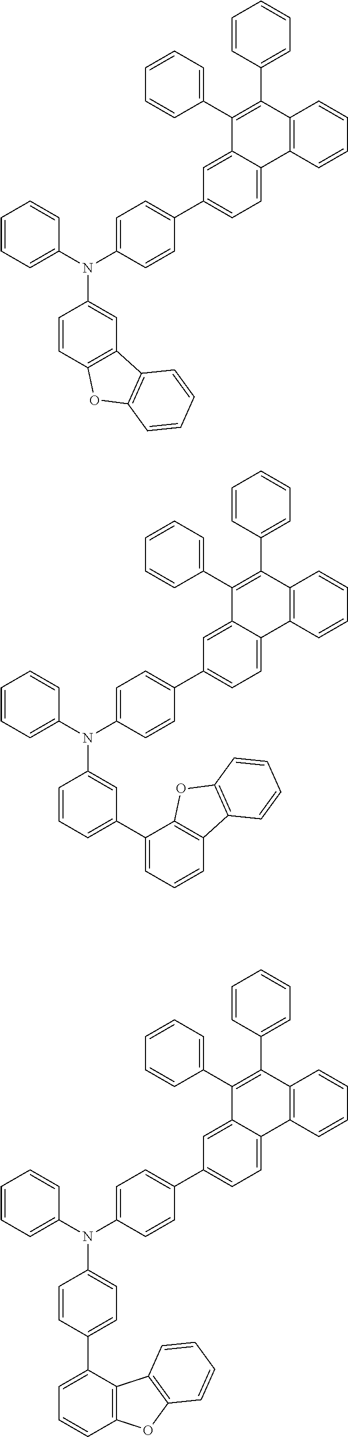 [SMILES: c1ccc(-c2c(-c3ccccc3)c3cc(-c4ccc(N(c5ccccc5)c5ccc6oc7ccccc7c6c5)cc4)ccc3c3ccccc23)cc1 ; c1ccc(-c2c(-c3ccccc3)c3cc(-c4ccc(N(c5ccccc5)c5cccc(-c6cccc7c6oc6ccccc67)c5)cc4)ccc3c3ccccc23)cc1 ; c1ccc(-c2c(-c3ccccc3)c3cc(-c4ccc(N(c5ccccc5)c5ccc(-c6cccc7oc8ccccc8c67)cc5)cc4)ccc3c3ccccc23)cc1]

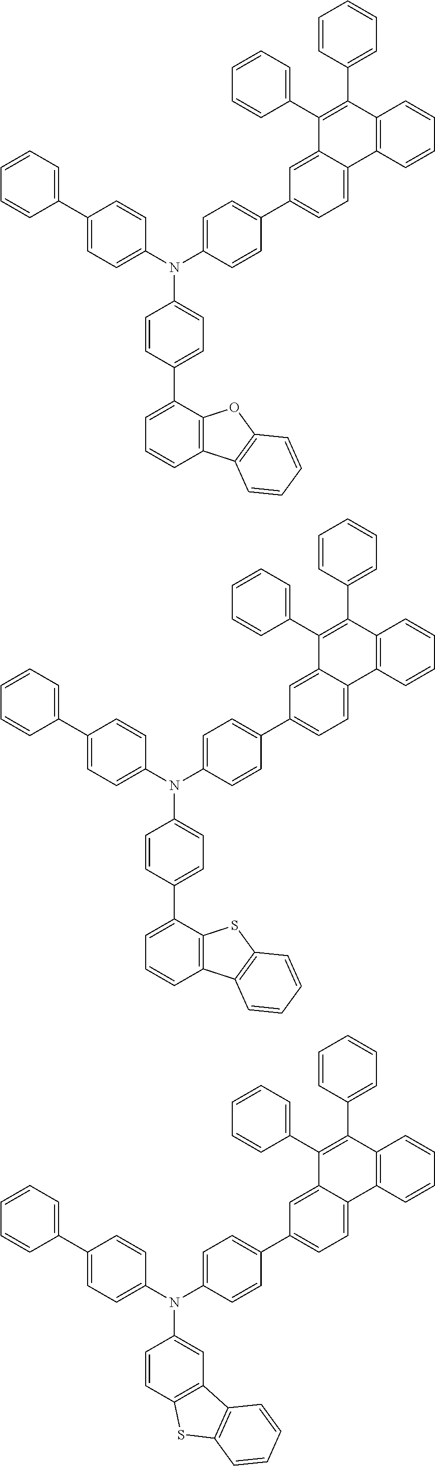 [SMILES: c1ccc(-c2ccc(N(c3ccc(-c4ccc5c(c4)c(-c4ccccc4)c(-c4ccccc4)c4ccccc45)cc3)c3ccc(-c4cccc5c4oc4ccccc45)cc3)cc2)cc1 ; c1ccc(-c2ccc(N(c3ccc(-c4ccc5c(c4)c(-c4ccccc4)c(-c4ccccc4)c4ccccc45)cc3)c3ccc(-c4cccc5c4sc4ccccc45)cc3)cc2)cc1 ; c1ccc(-c2ccc(N(c3ccc(-c4ccc5c(c4)c(-c4ccccc4)c(-c4ccccc4)c4ccccc45)cc3)c3ccc4sc5ccccc5c4c3)cc2)cc1]

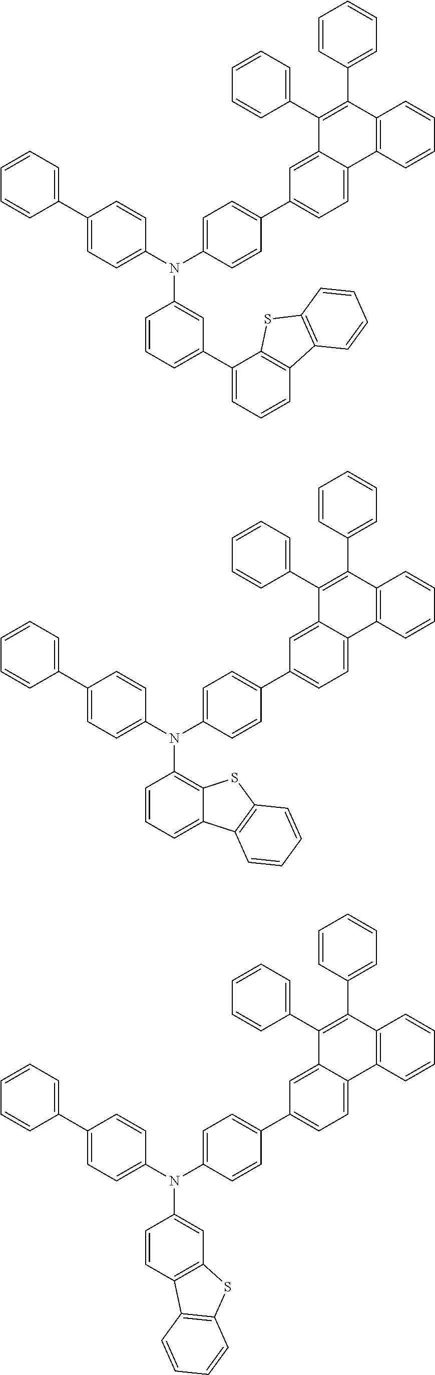 [SMILES: c1ccc(-c2ccc(N(c3ccc(-c4ccc5c(c4)c(-c4ccccc4)c(-c4ccccc4)c4ccccc45)cc3)c3cccc(-c4cccc5c4sc4ccccc45)c3)cc2)cc1 ; c1ccc(-c2ccc(N(c3ccc(-c4ccc5c(c4)c(-c4ccccc4)c(-c4ccccc4)c4ccccc45)cc3)c3cccc4c3sc3ccccc34)cc2)cc1 ; c1ccc(-c2ccc(N(c3ccc(-c4ccc5c(c4)c(-c4ccccc4)c(-c4ccccc4)c4ccccc45)cc3)c3ccc4c(c3)sc3ccccc34)cc2)cc1]

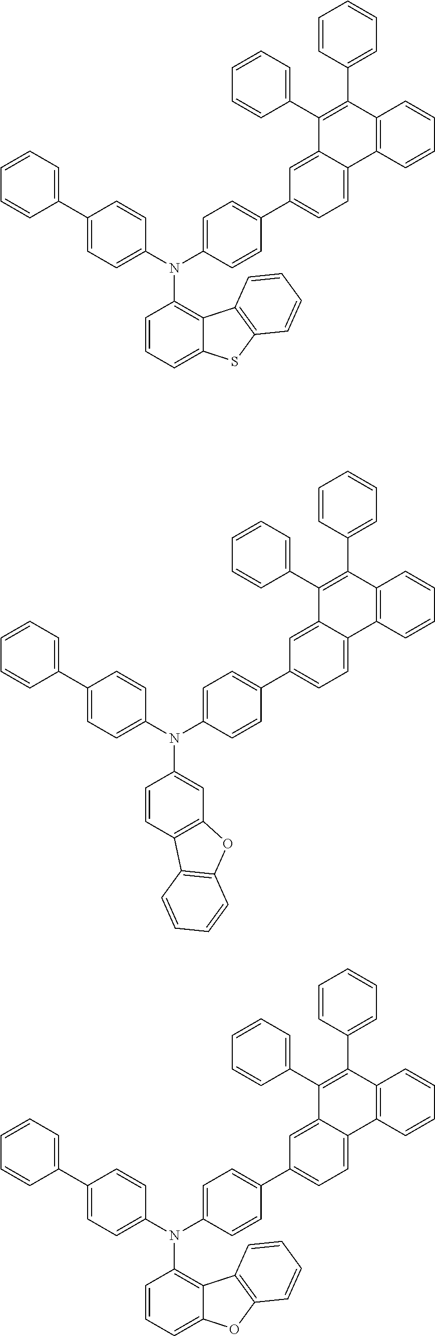 [SMILES: c1ccc(-c2ccc(N(c3ccc(-c4ccc5c(c4)c(-c4ccccc4)c(-c4ccccc4)c4ccccc45)cc3)c3cccc4sc5ccccc5c34)cc2)cc1 ; c1ccc(-c2ccc(N(c3ccc(-c4ccc5c(c4)c(-c4ccccc4)c(-c4ccccc4)c4ccccc45)cc3)c3ccc4c(c3)oc3ccccc34)cc2)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)cc2)cc1]

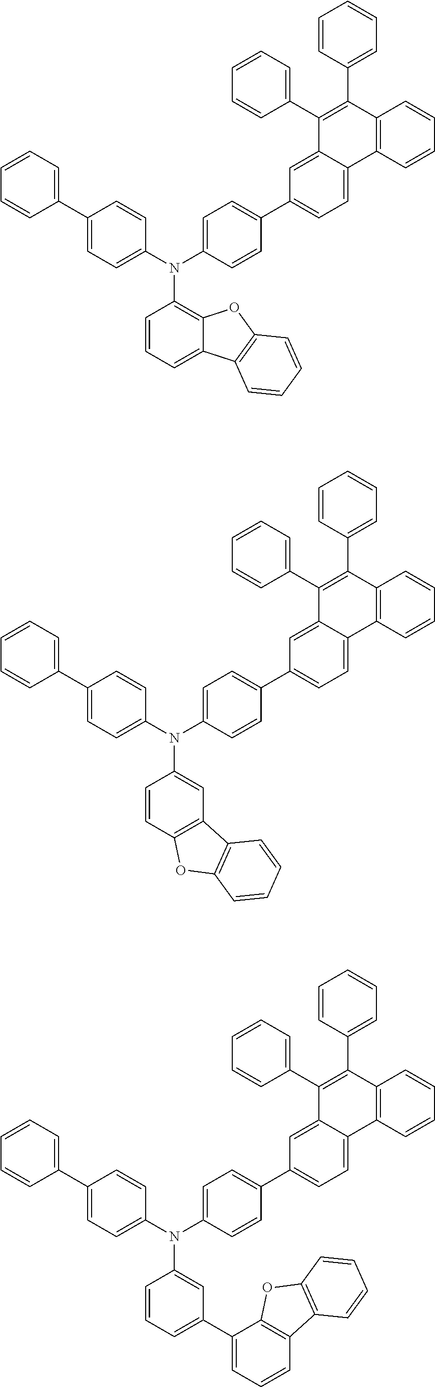 [SMILES: c1ccc(-c2ccc(N(c3ccc(-c4ccc5c(c4)c(-c4ccccc4)c(-c4ccccc4)c4ccccc45)cc3)c3cccc4c3oc3ccccc34)cc2)cc1 ; c1ccc(-c2ccc(N(c3ccc(-c4ccc5c(c4)c(-c4ccccc4)c(-c4ccccc4)c4ccccc45)cc3)c3ccc4oc5ccccc5c4c3)cc2)cc1 ; c1ccc(-c2ccc(N(c3ccc(-c4ccc5c(c4)c(-c4ccccc4)c(-c4ccccc4)c4ccccc45)cc3)c3cccc(-c4cccc5c4oc4ccccc45)c3)cc2)cc1]

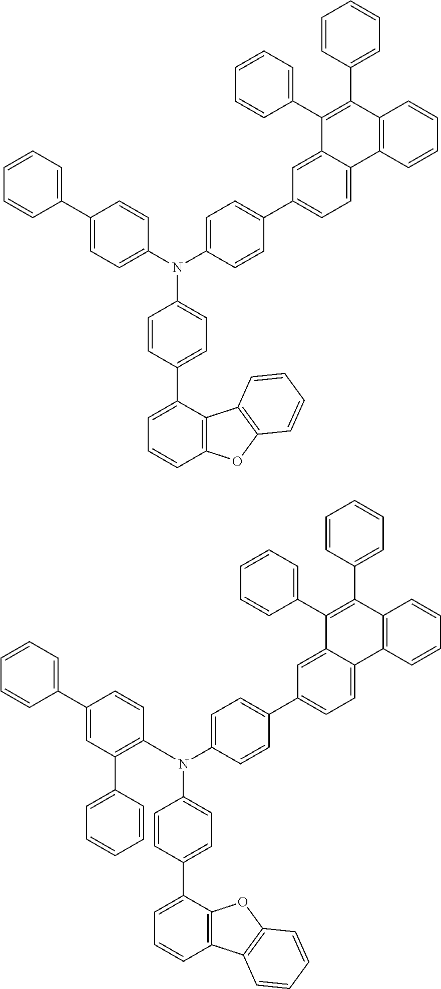 [SMILES: c1ccc(-c2ccc(N(c3ccc(-c4ccc5c(c4)c(-c4ccccc4)c(-c4ccccc4)c4ccccc45)cc3)c3ccc(-c4cccc5oc6ccccc6c45)cc3)cc2)cc1 ; c1ccc(-c2ccc(N(c3ccc(-c4ccc5c(c4)c(-c4ccccc4)c(-c4ccccc4)c4ccccc45)cc3)c3ccc(-c4cccc5c4oc4ccccc45)cc3)c(-c3ccccc3)c2)cc1]

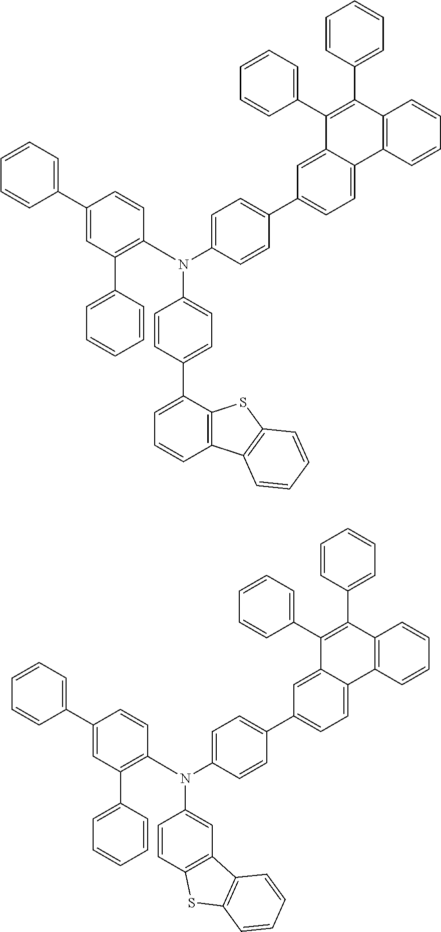 [SMILES: c1ccc(-c2ccc(N(c3ccc(-c4ccc5c(c4)c(-c4ccccc4)c(-c4ccccc4)c4ccccc45)cc3)c3ccc(-c4cccc5c4sc4ccccc45)cc3)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc4sc5ccccc5c4c3)c(-c3ccccc3)c2)cc1]

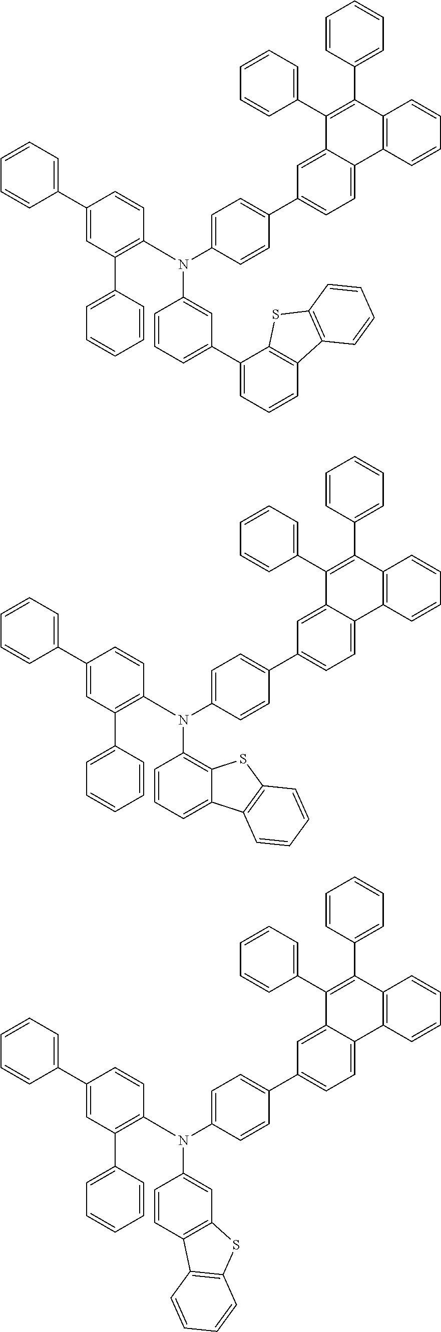 [SMILES: c1ccc(-c2ccc(N(c3ccc(-c4ccc5c(c4)c(-c4ccccc4)c(-c4ccccc4)c4ccccc45)cc3)c3cccc(-c4cccc5c4sc4ccccc45)c3)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3cccc4c3sc3ccccc34)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc4c(c3)sc3ccccc34)c(-c3ccccc3)c2)cc1]

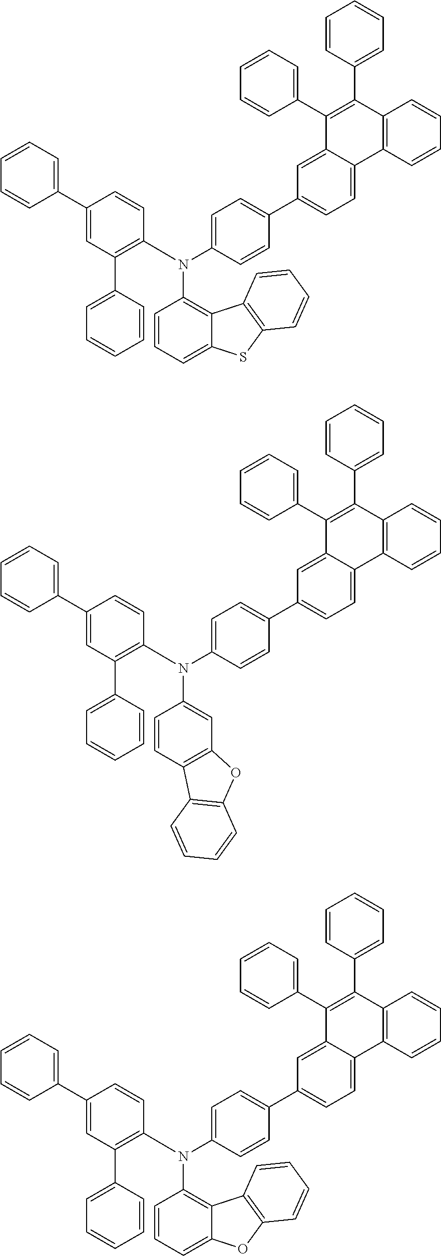 [SMILES: c1ccc(-c2ccc(N(c3ccc(-c4ccc5c(c4)c(-c4ccccc4)c(-c4ccccc4)c4ccccc45)cc3)c3cccc4sc5ccccc5c34)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc4c(c3)oc3ccccc34)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)c(-c3ccccc3)c2)cc1]

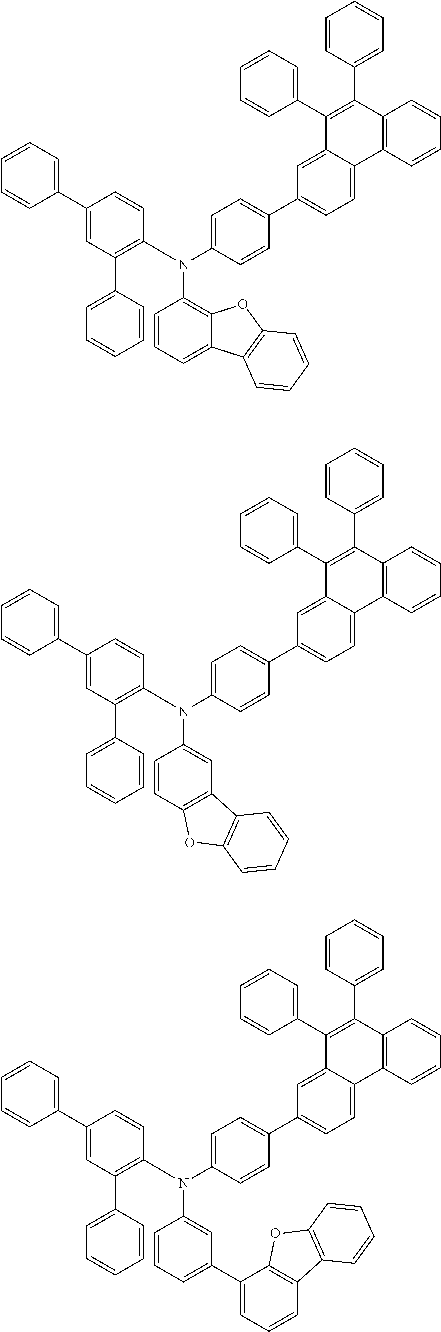 [SMILES: c1ccc(-c2ccc(N(c3ccc(-c4ccc5c(c4)c(-c4ccccc4)c(-c4ccccc4)c4ccccc45)cc3)c3cccc4c3oc3ccccc34)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc4oc5ccccc5c4c3)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3cccc(-c4cccc5c4oc4ccccc45)c3)c(-c3ccccc3)c2)cc1]

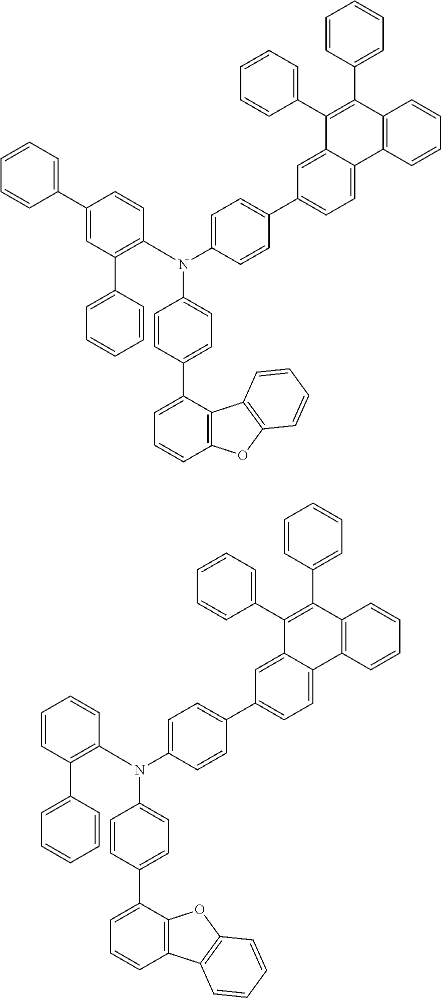 [SMILES: c1ccc(-c2ccc(N(c3ccc(-c4ccc5c(c4)c(-c4ccccc4)c(-c4ccccc4)c4ccccc45)cc3)c3ccc(-c4cccc5oc6ccccc6c45)cc3)c(-c3ccccc3)c2)cc1 ; c1ccc(-c2ccccc2N(c2ccc(-c3ccc4c(c3)c(-c3ccccc3)c(-c3ccccc3)c3ccccc34)cc2)c2ccc(-c3cccc4c3oc3ccccc34)cc2)cc1]

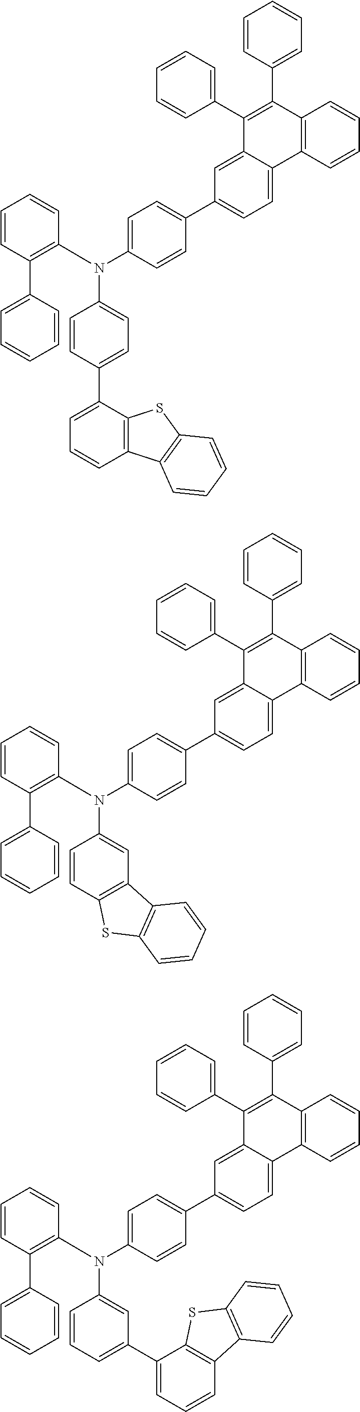 [SMILES: c1ccc(-c2ccccc2N(c2ccc(-c3ccc4c(c3)c(-c3ccccc3)c(-c3ccccc3)c3ccccc34)cc2)c2ccc(-c3cccc4c3sc3ccccc34)cc2)cc1 ; c1ccc(-c2ccccc2N(c2ccc(-c3ccc4c(c3)c(-c3ccccc3)c(-c3ccccc3)c3ccccc34)cc2)c2ccc3sc4ccccc4c3c2)cc1 ; c1ccc(-c2ccccc2N(c2ccc(-c3ccc4c(c3)c(-c3ccccc3)c(-c3ccccc3)c3ccccc34)cc2)c2cccc(-c3cccc4c3sc3ccccc34)c2)cc1]

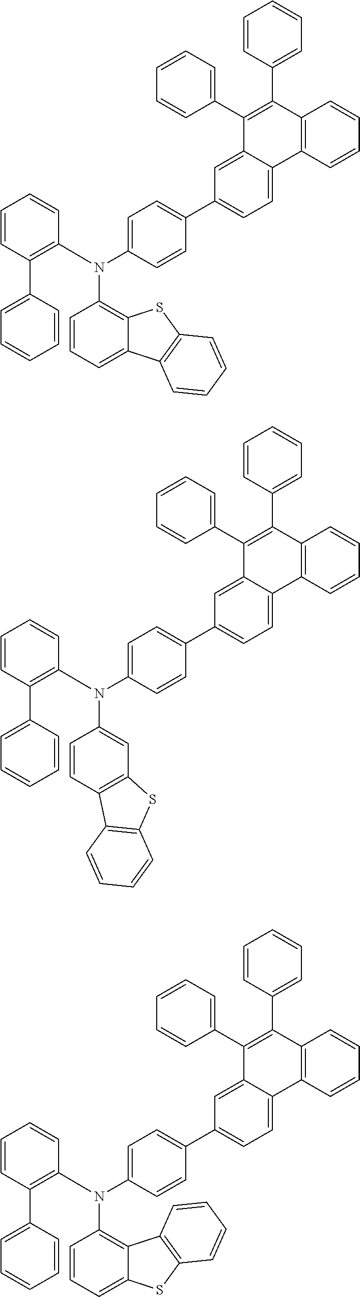 [SMILES: c1ccc(-c2ccccc2N(c2ccc(-c3ccc4c(c3)c(-c3ccccc3)c(-c3ccccc3)c3ccccc34)cc2)c2cccc3c2sc2ccccc23)cc1 ; c1ccc(-c2ccccc2N(c2ccc(-c3ccc4c(c3)c(-c3ccccc3)c(-c3ccccc3)c3ccccc34)cc2)c2ccc3c(c2)sc2ccccc23)cc1 ; c1ccc(-c2ccccc2N(c2ccc(-c3ccc4c(c3)c(-c3ccccc3)c(-c3ccccc3)c3ccccc34)cc2)c2cccc3sc4ccccc4c23)cc1]

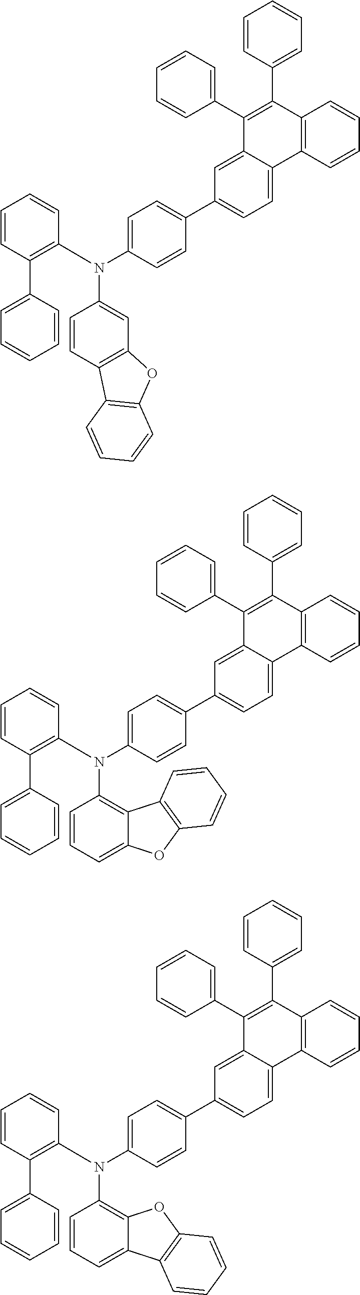 [SMILES: c1ccc(-c2ccccc2N(c2ccc(-c3ccc4c(c3)c(-c3ccccc3)c(-c3ccccc3)c3ccccc34)cc2)c2ccc3c(c2)oc2ccccc23)cc1 ; c1ccc(-c2ccccc2N(c2ccc(-c3ccc4c(c3)c(-c3ccccc3)c(-c3ccccc3)c3ccccc34)cc2)c2cccc3oc4ccccc4c23)cc1 ; c1ccc(-c2ccccc2N(c2ccc(-c3ccc4c(c3)c(-c3ccccc3)c(-c3ccccc3)c3ccccc34)cc2)c2cccc3c2oc2ccccc23)cc1]

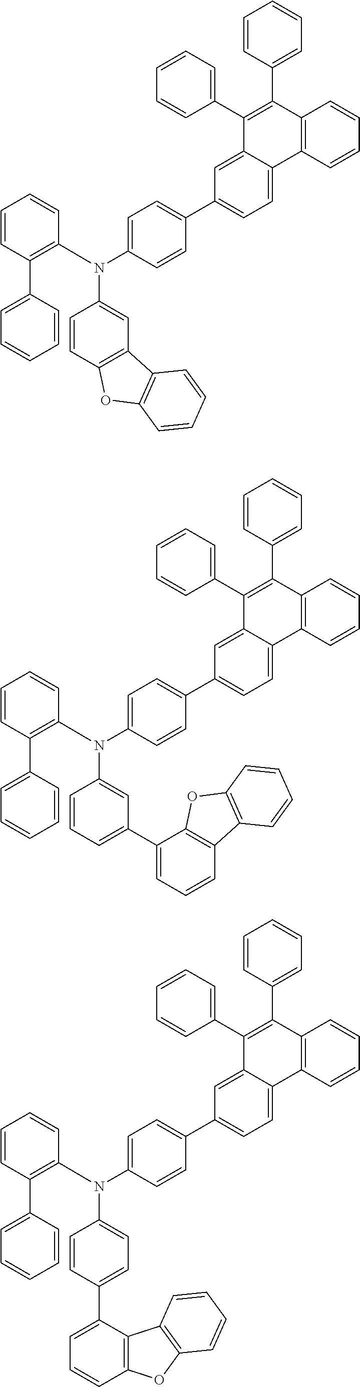 [SMILES: c1ccc(-c2ccccc2N(c2ccc(-c3ccc4c(c3)c(-c3ccccc3)c(-c3ccccc3)c3ccccc34)cc2)c2ccc3oc4ccccc4c3c2)cc1 ; c1ccc(-c2ccccc2N(c2ccc(-c3ccc4c(c3)c(-c3ccccc3)c(-c3ccccc3)c3ccccc34)cc2)c2cccc(-c3cccc4c3oc3ccccc34)c2)cc1 ; c1ccc(-c2ccccc2N(c2ccc(-c3ccc4c(c3)c(-c3ccccc3)c(-c3ccccc3)c3ccccc34)cc2)c2ccc(-c3cccc4oc5ccccc5c34)cc2)cc1]

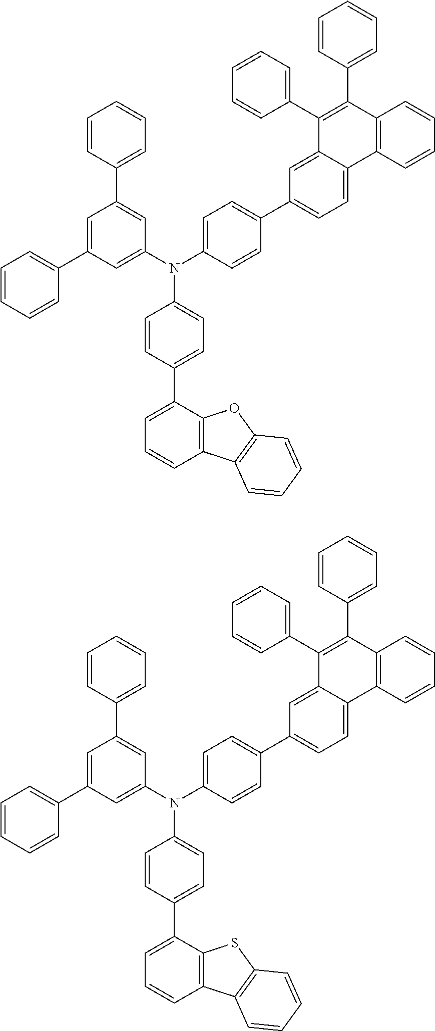 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc(-c4cccc5c4oc4ccccc45)cc3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc(-c4cccc5c4sc4ccccc45)cc3)c2)cc1]

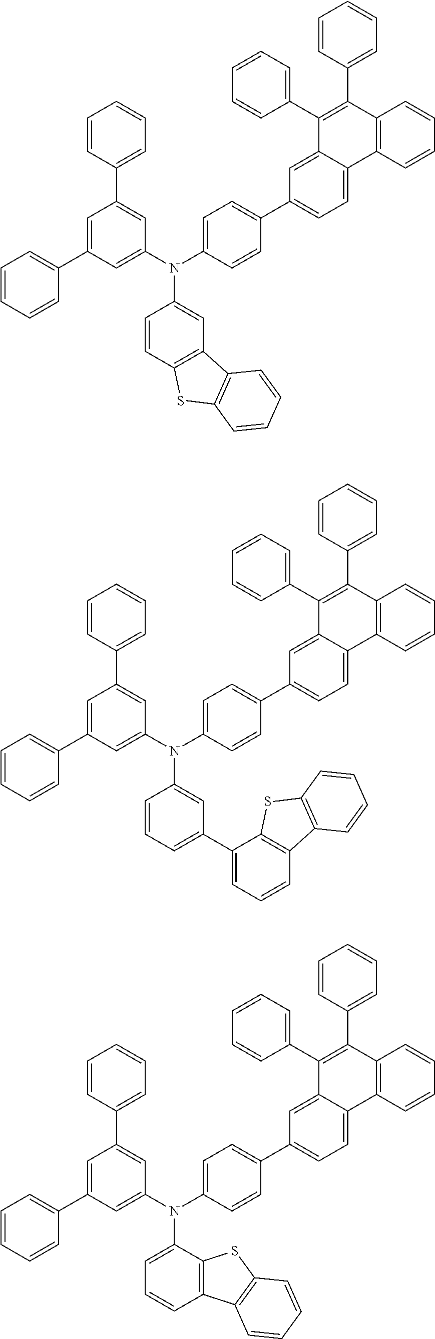 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc4sc5ccccc5c4c3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cccc(-c4cccc5c4sc4ccccc45)c3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cccc4c3sc3ccccc34)c2)cc1]

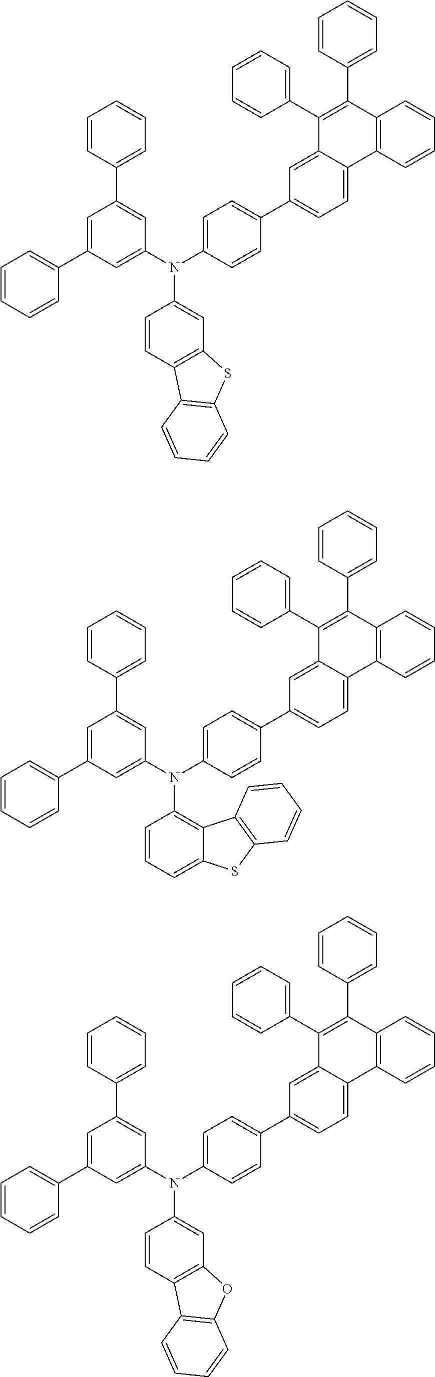 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc4c(c3)sc3ccccc34)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cccc4sc5ccccc5c34)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc4c(c3)oc3ccccc34)c2)cc1]

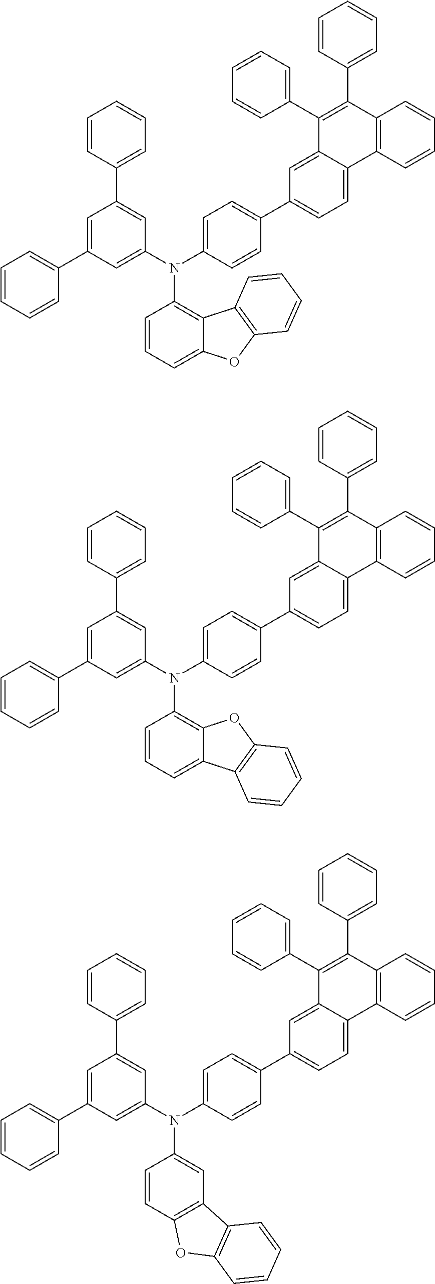 [SMILES: c1ccc(-c2cc(-c3ccccc3)cc(N(c3ccc(-c4ccc5c(c4)c(-c4ccccc4)c(-c4ccccc4)c4ccccc45)cc3)c3cccc4oc5ccccc5c34)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cccc4c3oc3ccccc34)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc4oc5ccccc5c4c3)c2)cc1]

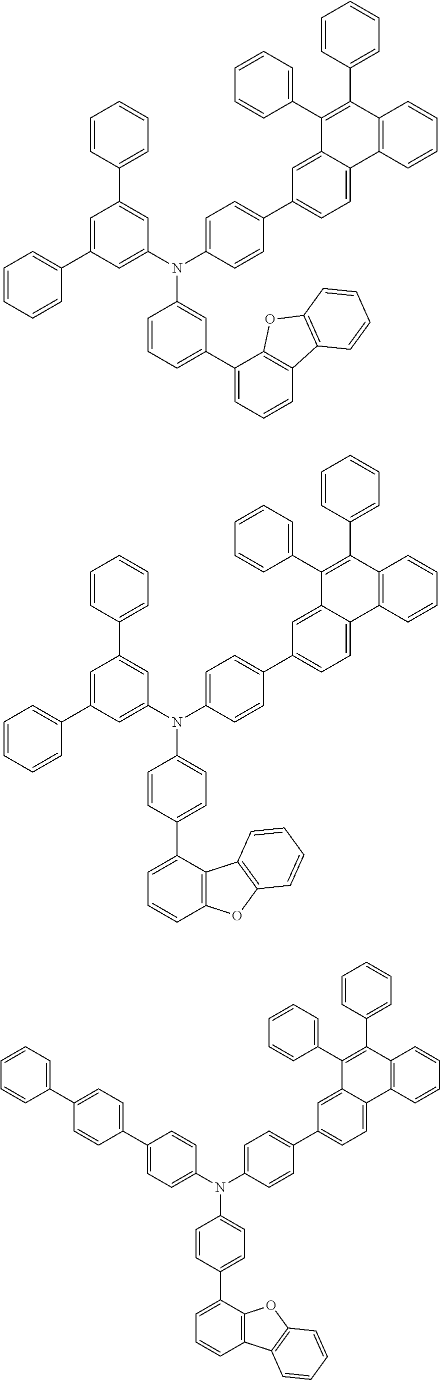 [SMILES: c1ccc(-c2cc(-c3ccccc3)cc(N(c3ccc(-c4ccc5c(c4)c(-c4ccccc4)c(-c4ccccc4)c4ccccc45)cc3)c3cccc(-c4cccc5c4oc4ccccc45)c3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc(-c4cccc5oc6ccccc6c45)cc3)c2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5cccc6c5oc5ccccc56)cc4)cc3)cc2)cc1]

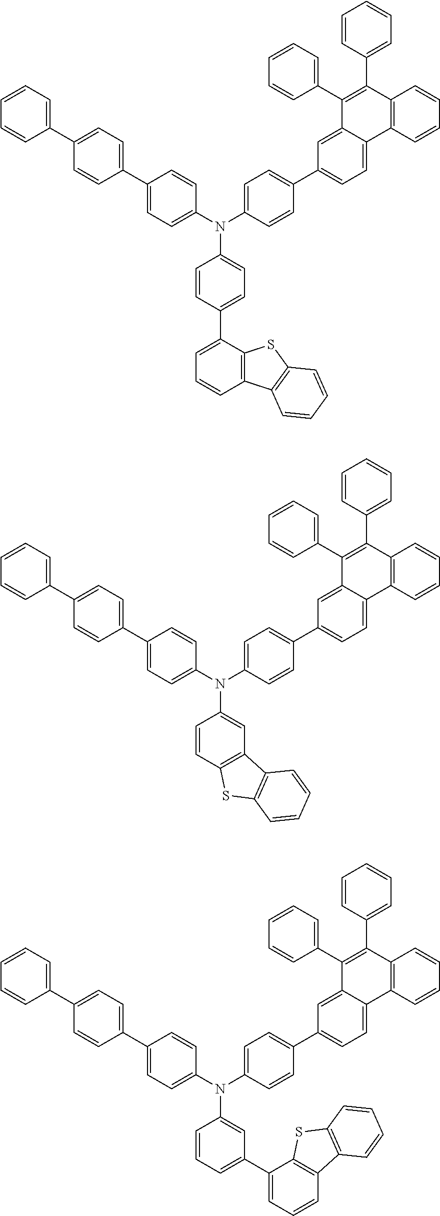 [SMILES: c1ccc(-c2ccc(-c3ccc(N(c4ccc(-c5ccc6c(c5)c(-c5ccccc5)c(-c5ccccc5)c5ccccc56)cc4)c4ccc(-c5cccc6c5sc5ccccc56)cc4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc5sc6ccccc6c5c4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4cccc(-c5cccc6c5sc5ccccc56)c4)cc3)cc2)cc1]

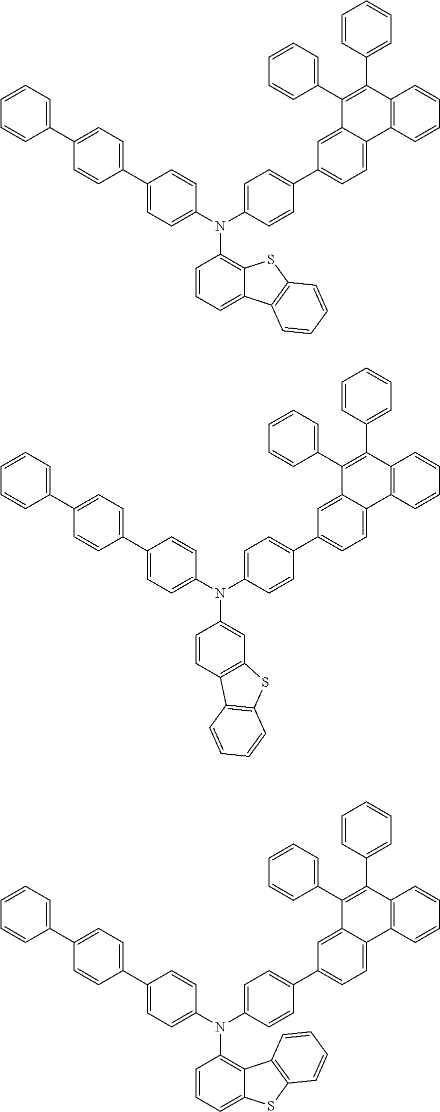 [SMILES: c1ccc(-c2ccc(-c3ccc(N(c4ccc(-c5ccc6c(c5)c(-c5ccccc5)c(-c5ccccc5)c5ccccc56)cc4)c4cccc5c4sc4ccccc45)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc5c(c4)sc4ccccc45)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4cccc5sc6ccccc6c45)cc3)cc2)cc1]

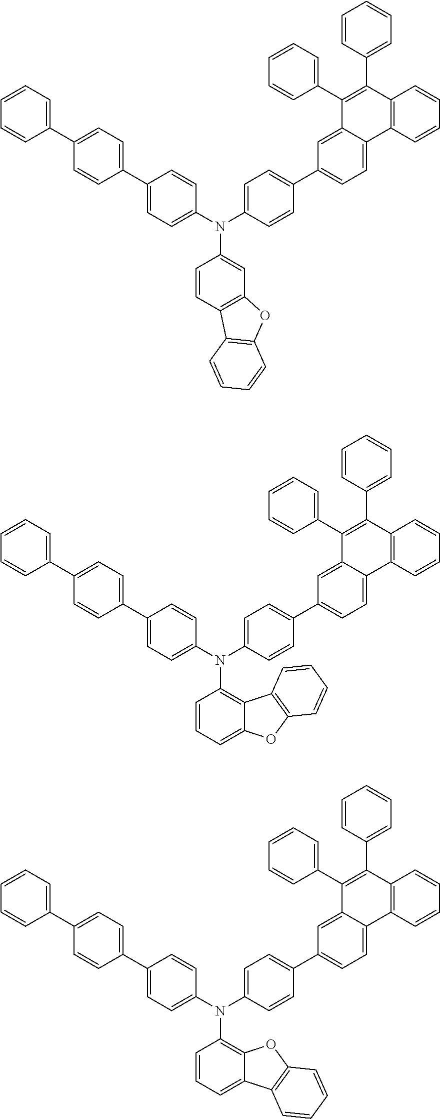 [SMILES: c1ccc(-c2ccc(-c3ccc(N(c4ccc(-c5ccc6c(c5)c(-c5ccccc5)c(-c5ccccc5)c5ccccc56)cc4)c4ccc5c(c4)oc4ccccc45)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4cccc5oc6ccccc6c45)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4cccc5c4oc4ccccc45)cc3)cc2)cc1]

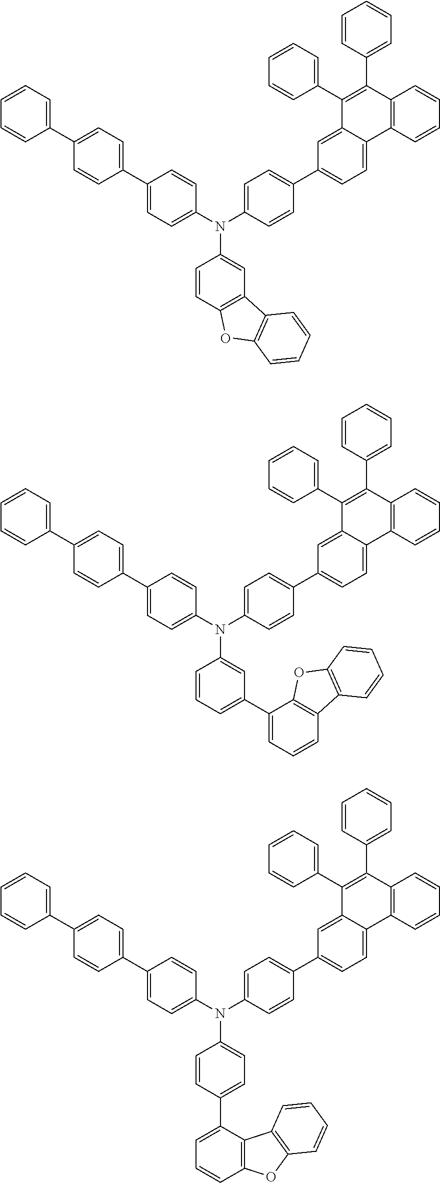 [SMILES: c1ccc(-c2ccc(-c3ccc(N(c4ccc(-c5ccc6c(c5)c(-c5ccccc5)c(-c5ccccc5)c5ccccc56)cc4)c4ccc5oc6ccccc6c5c4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4cccc(-c5cccc6c5oc5ccccc56)c4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5cccc6oc7ccccc7c56)cc4)cc3)cc2)cc1]

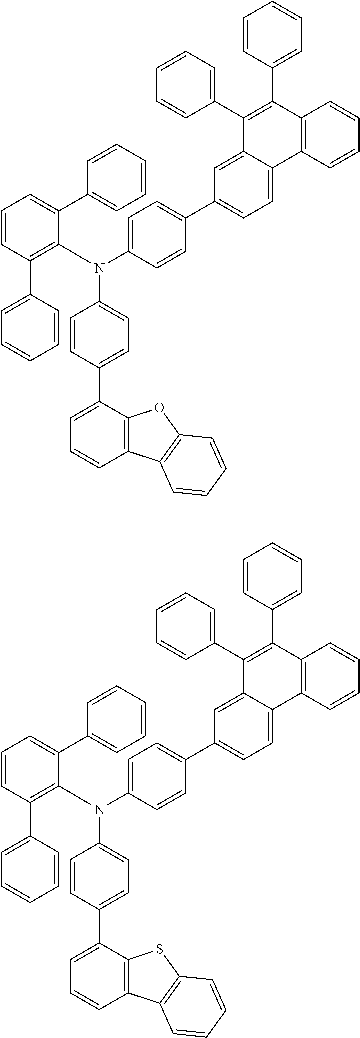 [SMILES: c1ccc(-c2cccc(-c3ccccc3)c2N(c2ccc(-c3ccc4c(c3)c(-c3ccccc3)c(-c3ccccc3)c3ccccc34)cc2)c2ccc(-c3cccc4c3oc3ccccc34)cc2)cc1 ; c1ccc(-c2cccc(-c3ccccc3)c2N(c2ccc(-c3ccc4c(c3)c(-c3ccccc3)c(-c3ccccc3)c3ccccc34)cc2)c2ccc(-c3cccc4c3sc3ccccc34)cc2)cc1]

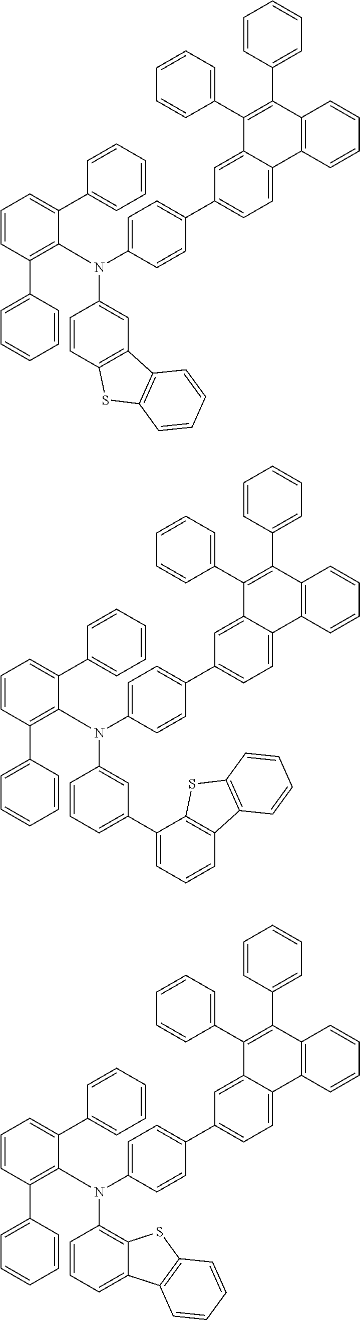 [SMILES: c1ccc(-c2cccc(-c3ccccc3)c2N(c2ccc(-c3ccc4c(c3)c(-c3ccccc3)c(-c3ccccc3)c3ccccc34)cc2)c2ccc3sc4ccccc4c3c2)cc1 ; c1ccc(-c2cccc(-c3ccccc3)c2N(c2ccc(-c3ccc4c(c3)c(-c3ccccc3)c(-c3ccccc3)c3ccccc34)cc2)c2cccc(-c3cccc4c3sc3ccccc34)c2)cc1 ; c1ccc(-c2cccc(-c3ccccc3)c2N(c2ccc(-c3ccc4c(c3)c(-c3ccccc3)c(-c3ccccc3)c3ccccc34)cc2)c2cccc3c2sc2ccccc23)cc1]

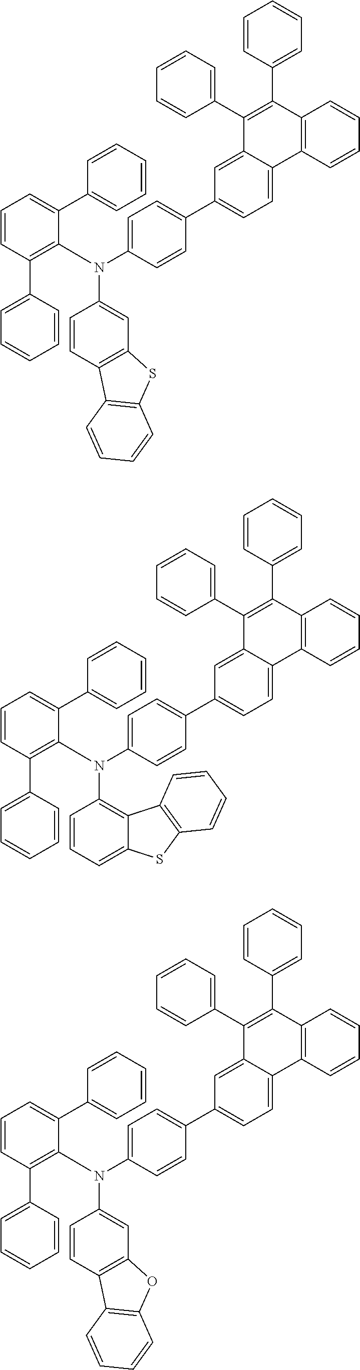 [SMILES: c1ccc(-c2cccc(-c3ccccc3)c2N(c2ccc(-c3ccc4c(c3)c(-c3ccccc3)c(-c3ccccc3)c3ccccc34)cc2)c2ccc3c(c2)sc2ccccc23)cc1 ; c1ccc(-c2cccc(-c3ccccc3)c2N(c2ccc(-c3ccc4c(c3)c(-c3ccccc3)c(-c3ccccc3)c3ccccc34)cc2)c2cccc3sc4ccccc4c23)cc1 ; c1ccc(-c2cccc(-c3ccccc3)c2N(c2ccc(-c3ccc4c(c3)c(-c3ccccc3)c(-c3ccccc3)c3ccccc34)cc2)c2ccc3c(c2)oc2ccccc23)cc1]

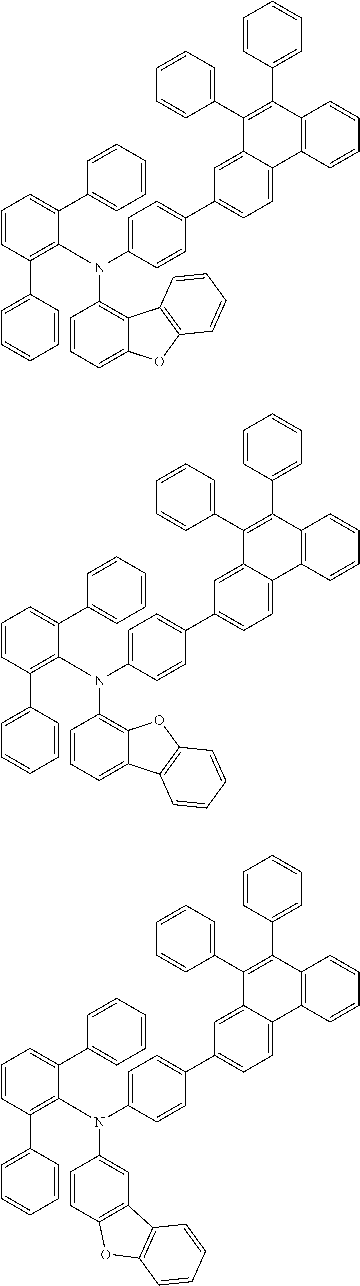 [SMILES: c1ccc(-c2cccc(-c3ccccc3)c2N(c2ccc(-c3ccc4c(c3)c(-c3ccccc3)c(-c3ccccc3)c3ccccc34)cc2)c2cccc3oc4ccccc4c23)cc1 ; c1ccc(-c2cccc(-c3ccccc3)c2N(c2ccc(-c3ccc4c(c3)c(-c3ccccc3)c(-c3ccccc3)c3ccccc34)cc2)c2cccc3c2oc2ccccc23)cc1 ; c1ccc(-c2cccc(-c3ccccc3)c2N(c2ccc(-c3ccc4c(c3)c(-c3ccccc3)c(-c3ccccc3)c3ccccc34)cc2)c2ccc3oc4ccccc4c3c2)cc1]

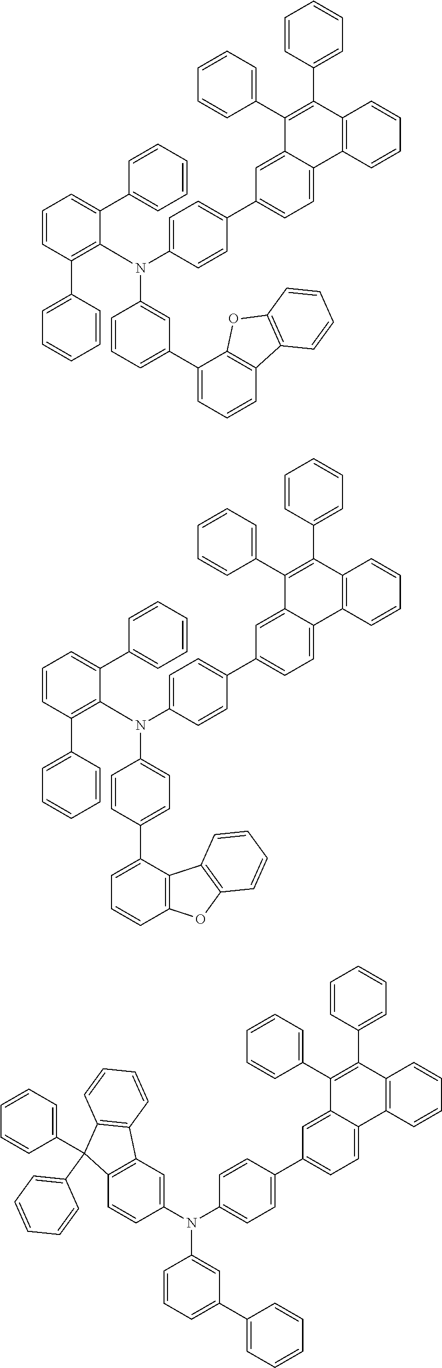 [SMILES: c1ccc(-c2cccc(-c3ccccc3)c2N(c2ccc(-c3ccc4c(c3)c(-c3ccccc3)c(-c3ccccc3)c3ccccc34)cc2)c2cccc(-c3cccc4c3oc3ccccc34)c2)cc1 ; c1ccc(-c2cccc(-c3ccccc3)c2N(c2ccc(-c3ccc4c(c3)c(-c3ccccc3)c(-c3ccccc3)c3ccccc34)cc2)c2ccc(-c3cccc4oc5ccccc5c34)cc2)cc1 ; c1ccc(-c2cccc(N(c3ccc(-c4ccc5c(c4)c(-c4ccccc4)c(-c4ccccc4)c4ccccc45)cc3)c3ccc4c(c3)-c3ccccc3C4(c3ccccc3)c3ccccc3)c2)cc1]

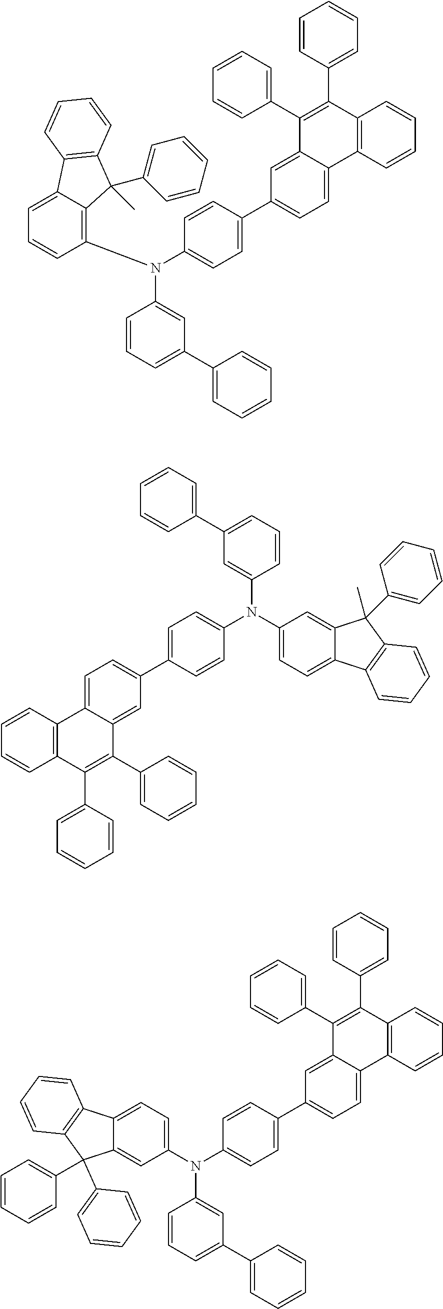 [SMILES: CC1(c2ccccc2)c2ccccc2-c2cccc(N(c3ccc(-c4ccc5c(c4)c(-c4ccccc4)c(-c4ccccc4)c4ccccc45)cc3)c3cccc(-c4ccccc4)c3)c21 ; CC1(c2ccccc2)c2ccccc2-c2ccc(N(c3ccc(-c4ccc5c(c4)c(-c4ccccc4)c(-c4ccccc4)c4ccccc45)cc3)c3cccc(-c4ccccc4)c3)cc21 ; c1ccc(-c2cccc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)c2)cc1]

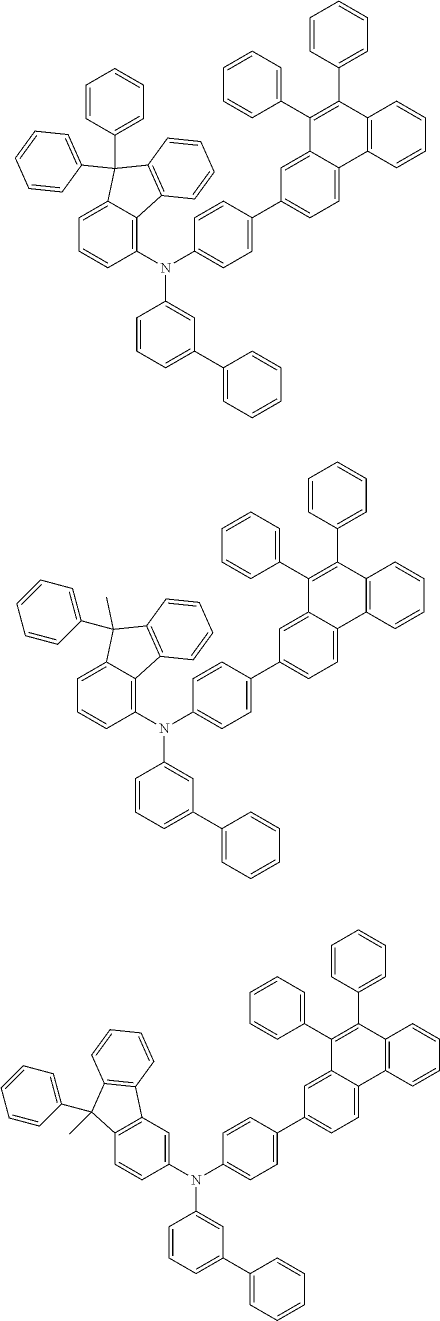 [SMILES: c1ccc(-c2cccc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)c2)cc1 ; CC1(c2ccccc2)c2ccccc2-c2c(N(c3ccc(-c4ccc5c(c4)c(-c4ccccc4)c(-c4ccccc4)c4ccccc45)cc3)c3cccc(-c4ccccc4)c3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3cccc(-c4ccccc4)c3)ccc21]

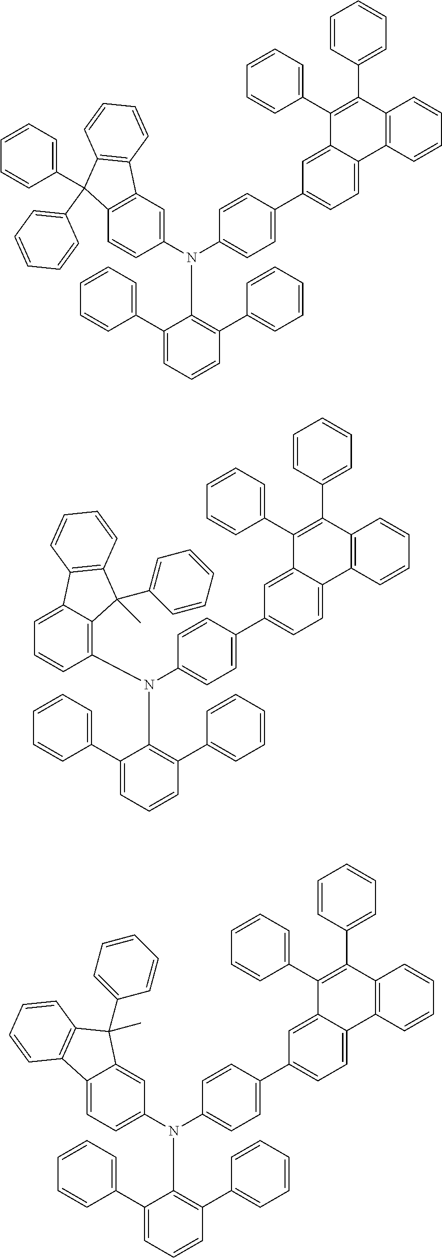 [SMILES: c1ccc(-c2cccc(-c3ccccc3)c2N(c2ccc(-c3ccc4c(c3)c(-c3ccccc3)c(-c3ccccc3)c3ccccc34)cc2)c2ccc3c(c2)-c2ccccc2C3(c2ccccc2)c2ccccc2)cc1 ; CC1(c2ccccc2)c2ccccc2-c2cccc(N(c3ccc(-c4ccc5c(c4)c(-c4ccccc4)c(-c4ccccc4)c4ccccc45)cc3)c3c(-c4ccccc4)cccc3-c3ccccc3)c21 ; CC1(c2ccccc2)c2ccccc2-c2ccc(N(c3ccc(-c4ccc5c(c4)c(-c4ccccc4)c(-c4ccccc4)c4ccccc45)cc3)c3c(-c4ccccc4)cccc3-c3ccccc3)cc21]

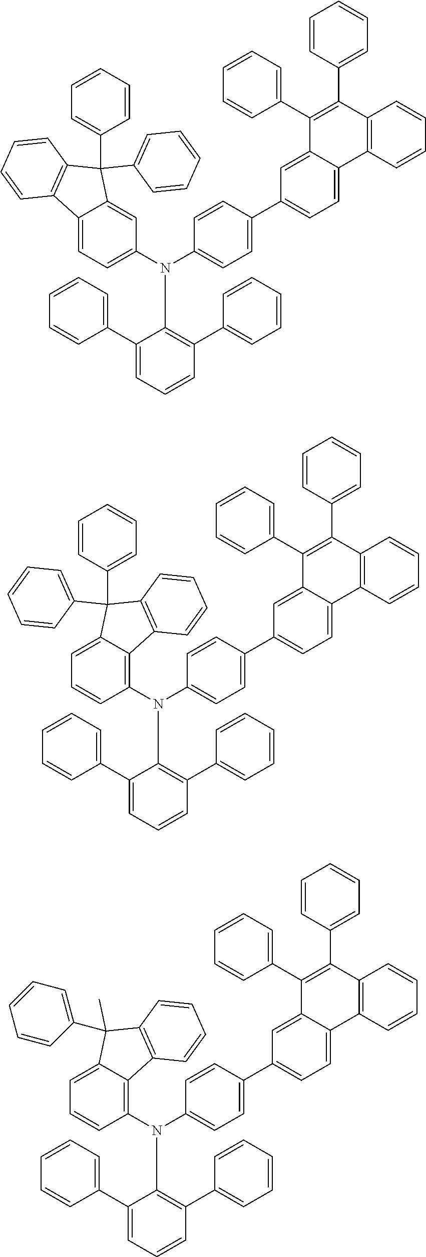 [SMILES: c1ccc(-c2cccc(-c3ccccc3)c2N(c2ccc(-c3ccc4c(c3)c(-c3ccccc3)c(-c3ccccc3)c3ccccc34)cc2)c2ccc3c(c2)C(c2ccccc2)(c2ccccc2)c2ccccc2-3)cc1 ; c1ccc(-c2cccc(-c3ccccc3)c2N(c2ccc(-c3ccc4c(c3)c(-c3ccccc3)c(-c3ccccc3)c3ccccc34)cc2)c2cccc3c2-c2ccccc2C3(c2ccccc2)c2ccccc2)cc1 ; CC1(c2ccccc2)c2ccccc2-c2c(N(c3ccc(-c4ccc5c(c4)c(-c4ccccc4)c(-c4ccccc4)c4ccccc45)cc3)c3c(-c4ccccc4)cccc3-c3ccccc3)cccc21]

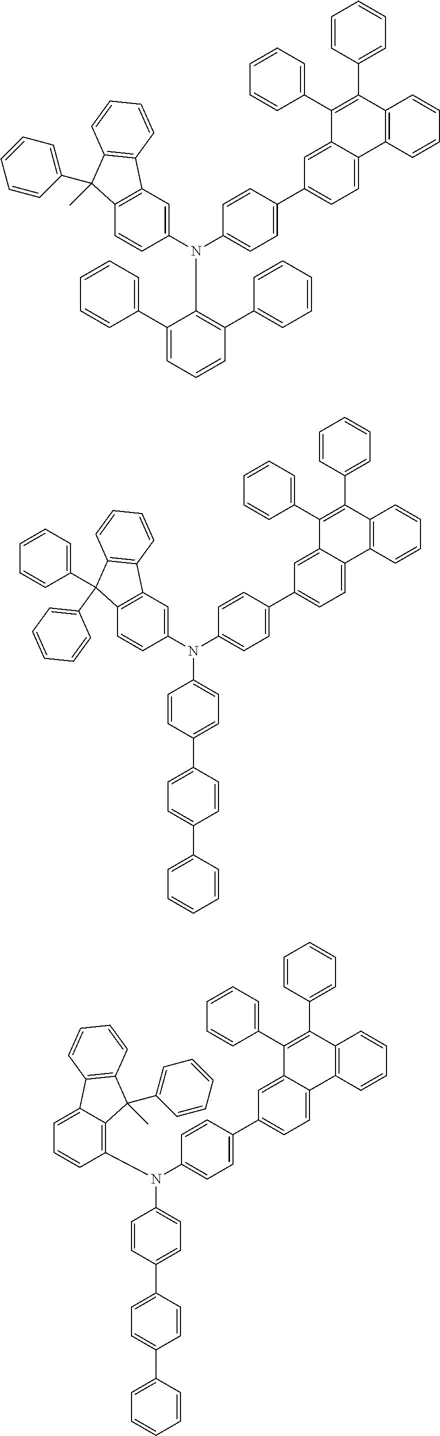 [SMILES: CC1(c2ccccc2)c2ccccc2-c2cc(N(c3ccc(-c4ccc5c(c4)c(-c4ccccc4)c(-c4ccccc4)c4ccccc45)cc3)c3c(-c4ccccc4)cccc3-c3ccccc3)ccc21 ; c1ccc(-c2ccc(-c3ccc(N(c4ccc(-c5ccc6c(c5)c(-c5ccccc5)c(-c5ccccc5)c5ccccc56)cc4)c4ccc5c(c4)-c4ccccc4C5(c4ccccc4)c4ccccc4)cc3)cc2)cc1 ; CC1(c2ccccc2)c2ccccc2-c2cccc(N(c3ccc(-c4ccc(-c5ccccc5)cc4)cc3)c3ccc(-c4ccc5c(c4)c(-c4ccccc4)c(-c4ccccc4)c4ccccc45)cc3)c21]

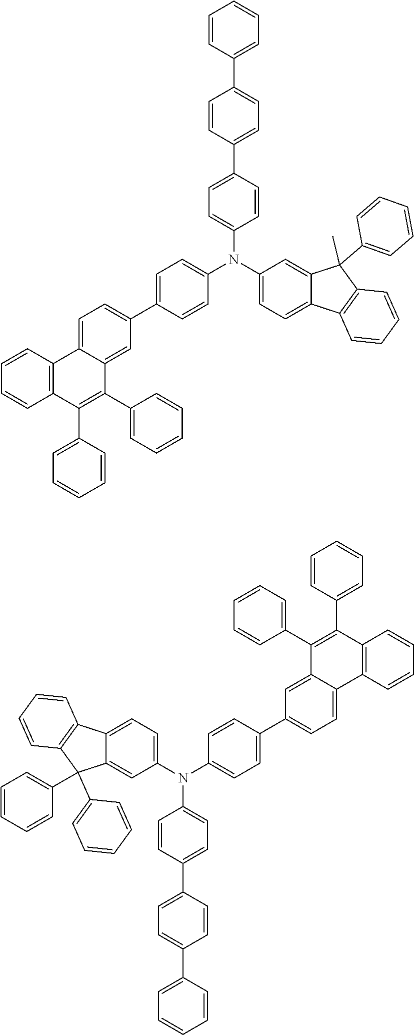 [SMILES: CC1(c2ccccc2)c2ccccc2-c2ccc(N(c3ccc(-c4ccc(-c5ccccc5)cc4)cc3)c3ccc(-c4ccc5c(c4)c(-c4ccccc4)c(-c4ccccc4)c4ccccc45)cc3)cc21 ; c1ccc(-c2ccc(-c3ccc(N(c4ccc(-c5ccc6c(c5)c(-c5ccccc5)c(-c5ccccc5)c5ccccc56)cc4)c4ccc5c(c4)C(c4ccccc4)(c4ccccc4)c4ccccc4-5)cc3)cc2)cc1]

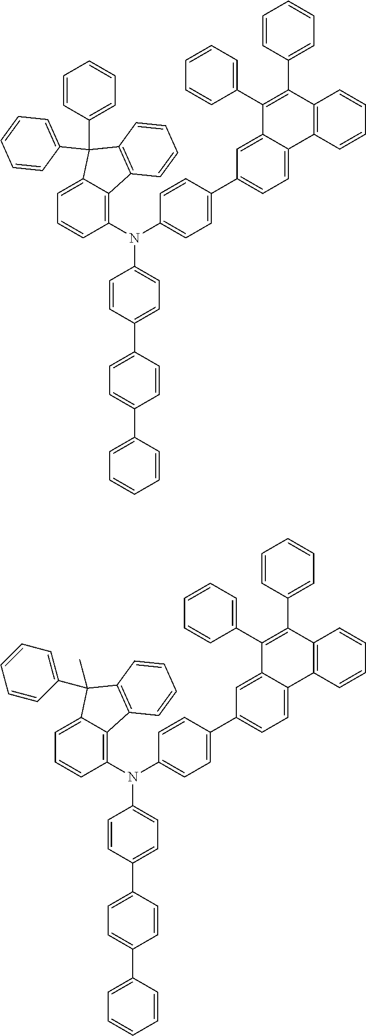 [SMILES: c1ccc(-c2ccc(-c3ccc(N(c4ccc(-c5ccc6c(c5)c(-c5ccccc5)c(-c5ccccc5)c5ccccc56)cc4)c4cccc5c4-c4ccccc4C5(c4ccccc4)c4ccccc4)cc3)cc2)cc1 ; CC1(c2ccccc2)c2ccccc2-c2c(N(c3ccc(-c4ccc(-c5ccccc5)cc4)cc3)c3ccc(-c4ccc5c(c4)c(-c4ccccc4)c(-c4ccccc4)c4ccccc45)cc3)cccc21]

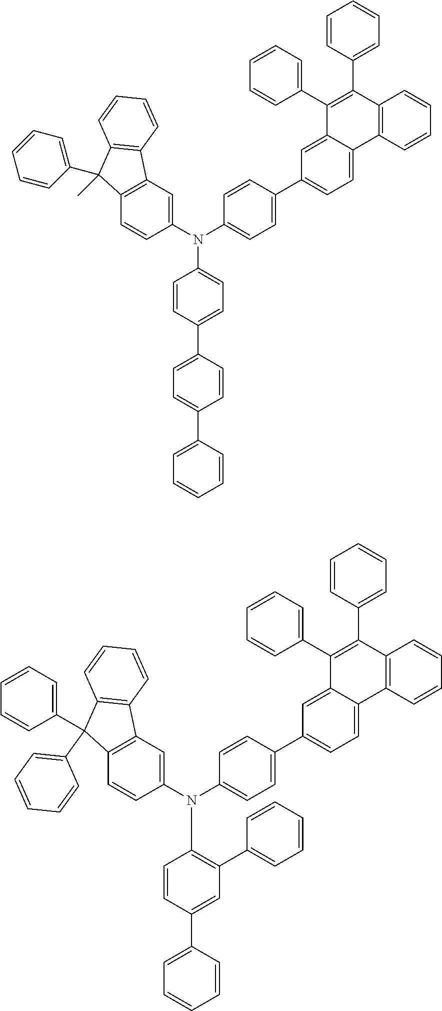 [SMILES: CC1(c2ccccc2)c2ccccc2-c2cc(N(c3ccc(-c4ccc(-c5ccccc5)cc4)cc3)c3ccc(-c4ccc5c(c4)c(-c4ccccc4)c(-c4ccccc4)c4ccccc45)cc3)ccc21 ; c1ccc(-c2ccc(N(c3ccc(-c4ccc5c(c4)c(-c4ccccc4)c(-c4ccccc4)c4ccccc45)cc3)c3ccc4c(c3)-c3ccccc3C4(c3ccccc3)c3ccccc3)c(-c3ccccc3)c2)cc1]

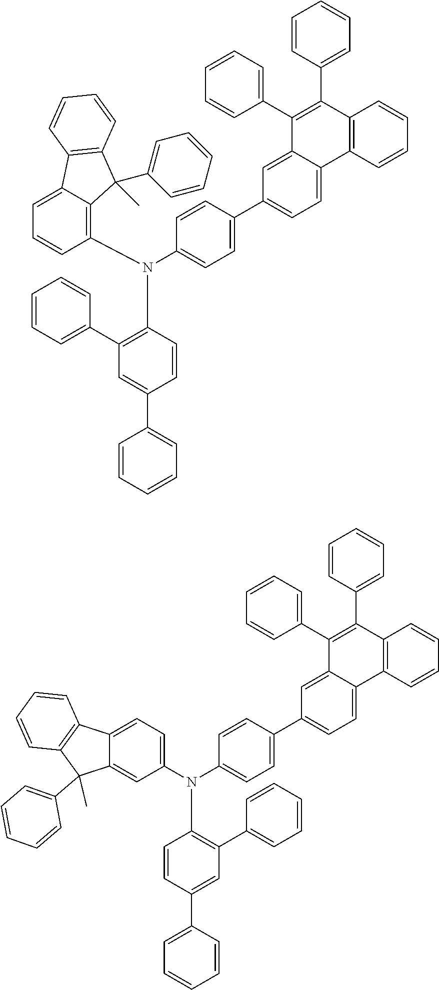 [SMILES: CC1(c2ccccc2)c2ccccc2-c2cccc(N(c3ccc(-c4ccc5c(c4)c(-c4ccccc4)c(-c4ccccc4)c4ccccc45)cc3)c3ccc(-c4ccccc4)cc3-c3ccccc3)c21 ; CC1(c2ccccc2)c2ccccc2-c2ccc(N(c3ccc(-c4ccc5c(c4)c(-c4ccccc4)c(-c4ccccc4)c4ccccc45)cc3)c3ccc(-c4ccccc4)cc3-c3ccccc3)cc21]

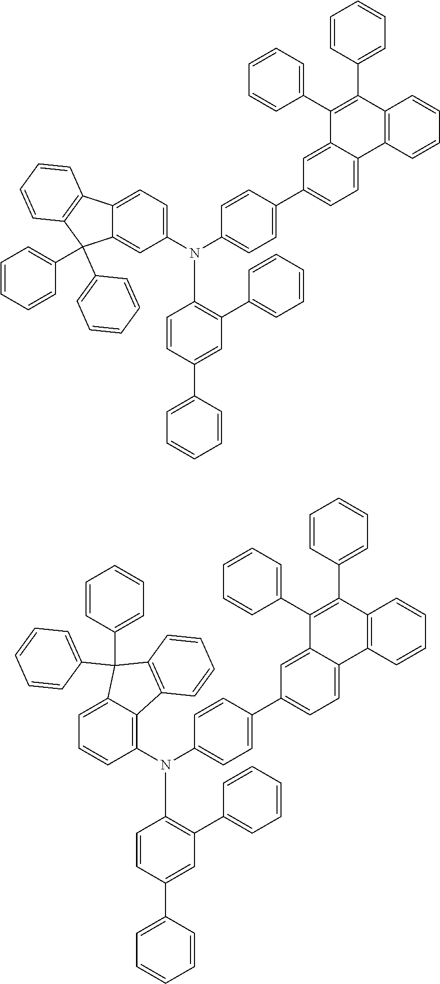 [SMILES: c1ccc(-c2ccc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)c(-c3ccccc3)c2)cc1]

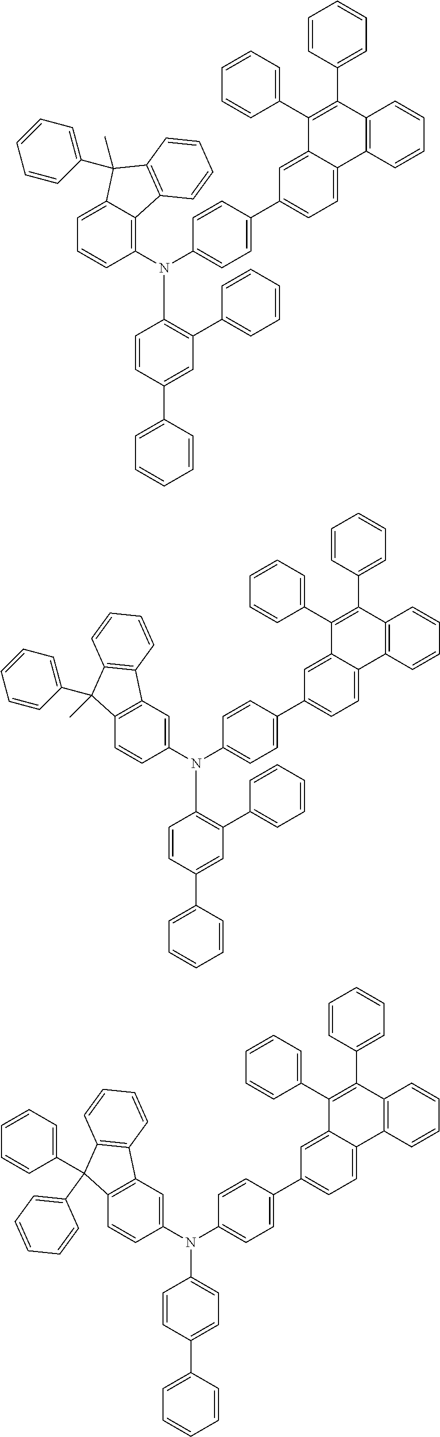 [SMILES: CC1(c2ccccc2)c2ccccc2-c2c(N(c3ccc(-c4ccc5c(c4)c(-c4ccccc4)c(-c4ccccc4)c4ccccc45)cc3)c3ccc(-c4ccccc4)cc3-c3ccccc3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3ccc(-c4ccccc4)cc3-c3ccccc3)ccc21 ; c1ccc(-c2ccc(N(c3ccc(-c4ccc5c(c4)c(-c4ccccc4)c(-c4ccccc4)c4ccccc45)cc3)c3ccc4c(c3)-c3ccccc3C4(c3ccccc3)c3ccccc3)cc2)cc1]

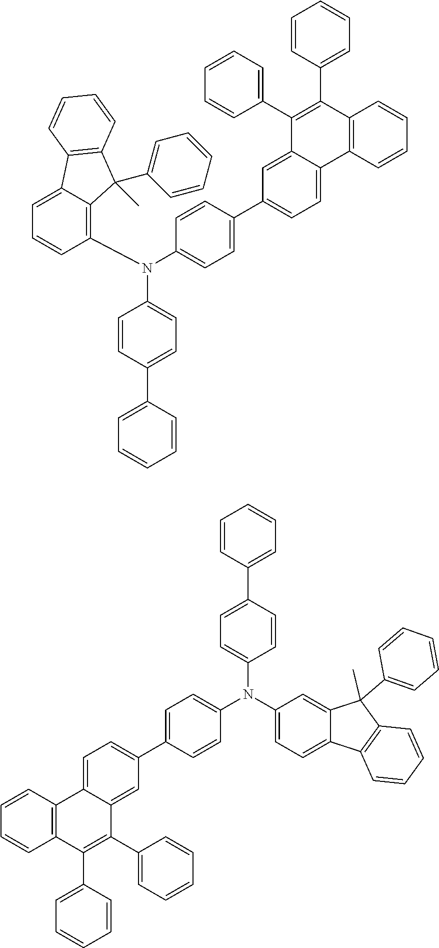 [SMILES: CC1(c2ccccc2)c2ccccc2-c2cccc(N(c3ccc(-c4ccccc4)cc3)c3ccc(-c4ccc5c(c4)c(-c4ccccc4)c(-c4ccccc4)c4ccccc45)cc3)c21 ; CC1(c2ccccc2)c2ccccc2-c2ccc(N(c3ccc(-c4ccccc4)cc3)c3ccc(-c4ccc5c(c4)c(-c4ccccc4)c(-c4ccccc4)c4ccccc45)cc3)cc21]

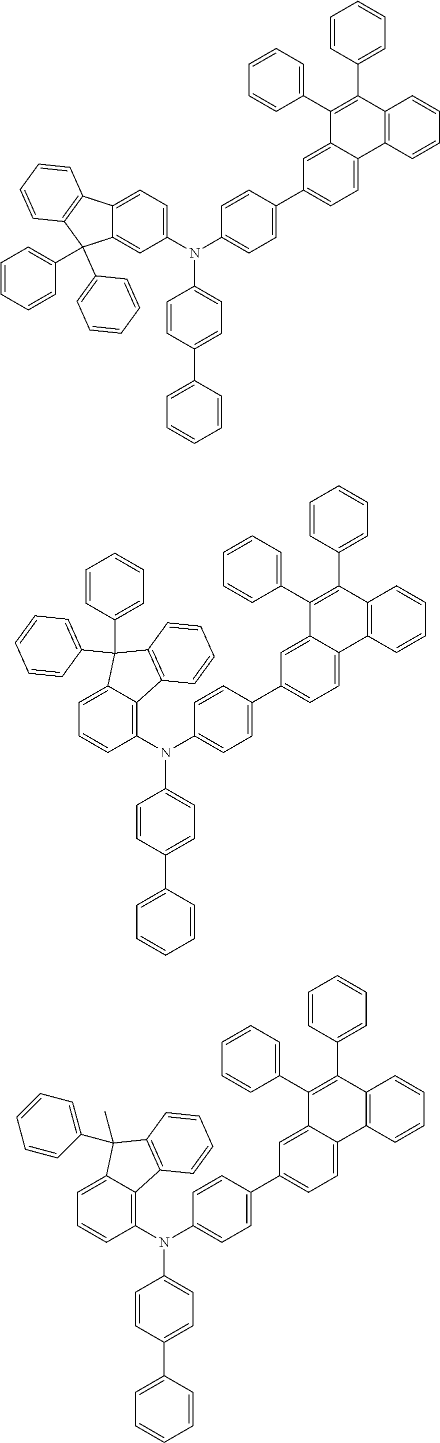 [SMILES: c1ccc(-c2ccc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)cc2)cc1 ; c1ccc(-c2ccc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)cc2)cc1 ; CC1(c2ccccc2)c2ccccc2-c2c(N(c3ccc(-c4ccccc4)cc3)c3ccc(-c4ccc5c(c4)c(-c4ccccc4)c(-c4ccccc4)c4ccccc45)cc3)cccc21]

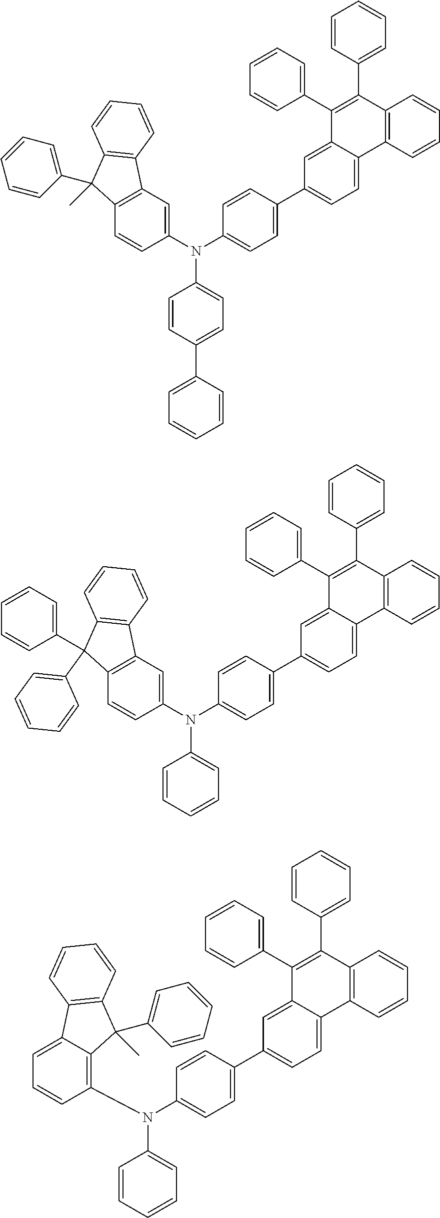 [SMILES: CC1(c2ccccc2)c2ccccc2-c2cc(N(c3ccc(-c4ccccc4)cc3)c3ccc(-c4ccc5c(c4)c(-c4ccccc4)c(-c4ccccc4)c4ccccc45)cc3)ccc21 ; c1ccc(-c2c(-c3ccccc3)c3cc(-c4ccc(N(c5ccccc5)c5ccc6c(c5)-c5ccccc5C6(c5ccccc5)c5ccccc5)cc4)ccc3c3ccccc23)cc1 ; CC1(c2ccccc2)c2ccccc2-c2cccc(N(c3ccccc3)c3ccc(-c4ccc5c(c4)c(-c4ccccc4)c(-c4ccccc4)c4ccccc45)cc3)c21]

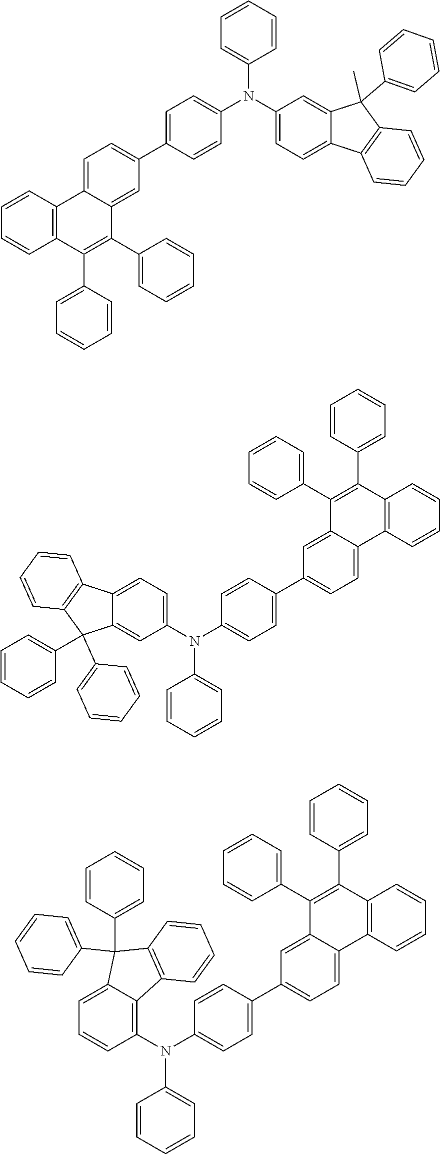 [SMILES: CC1(c2ccccc2)c2ccccc2-c2ccc(N(c3ccccc3)c3ccc(-c4ccc5c(c4)c(-c4ccccc4)c(-c4ccccc4)c4ccccc45)cc3)cc21 ; c1ccc(-c2c(-c3ccccc3)c3cc(-c4ccc(N(c5ccccc5)c5ccc6c(c5)C(c5ccccc5)(c5ccccc5)c5ccccc5-6)cc4)ccc3c3ccccc23)cc1 ; c1ccc(-c2c(-c3ccccc3)c3cc(-c4ccc(N(c5ccccc5)c5cccc6c5-c5ccccc5C6(c5ccccc5)c5ccccc5)cc4)ccc3c3ccccc23)cc1]

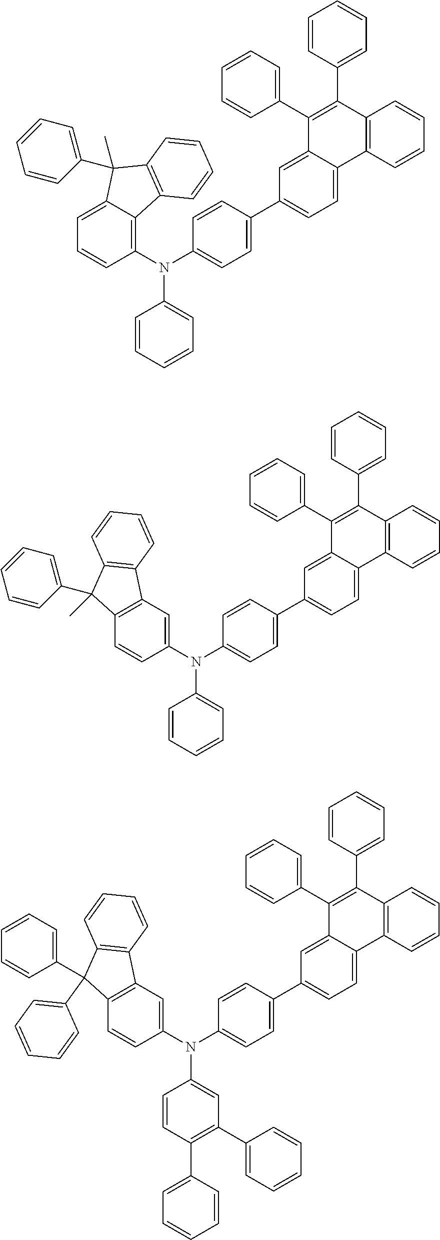 [SMILES: CC1(c2ccccc2)c2ccccc2-c2c(N(c3ccccc3)c3ccc(-c4ccc5c(c4)c(-c4ccccc4)c(-c4ccccc4)c4ccccc45)cc3)cccc21 ; CC1(c2ccccc2)c2ccccc2-c2cc(N(c3ccccc3)c3ccc(-c4ccc5c(c4)c(-c4ccccc4)c(-c4ccccc4)c4ccccc45)cc3)ccc21 ; c1ccc(-c2ccc(N(c3ccc(-c4ccc5c(c4)c(-c4ccccc4)c(-c4ccccc4)c4ccccc45)cc3)c3ccc4c(c3)-c3ccccc3C4(c3ccccc3)c3ccccc3)cc2-c2ccccc2)cc1]

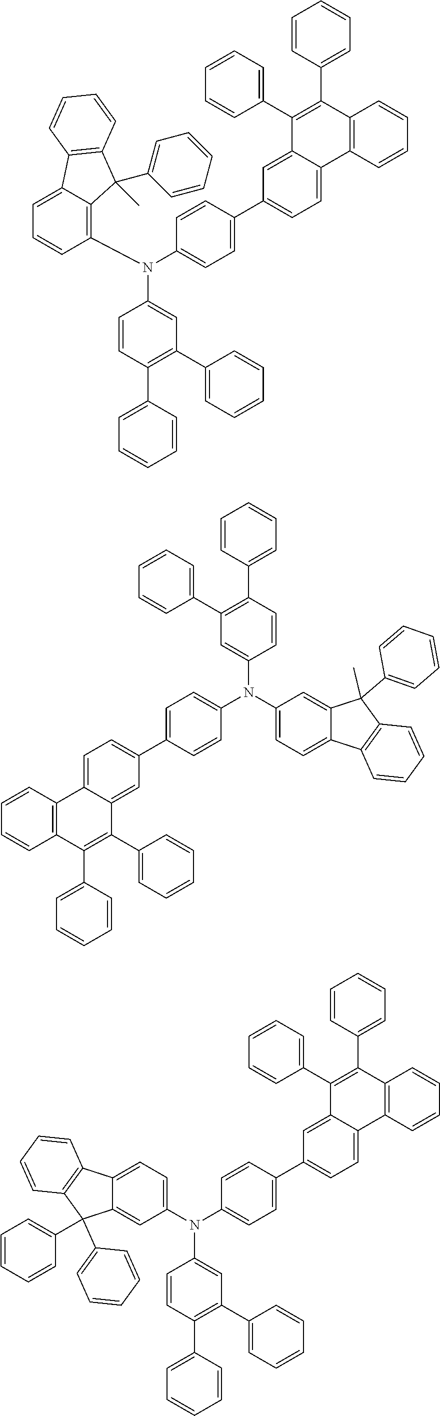 [SMILES: CC1(c2ccccc2)c2ccccc2-c2cccc(N(c3ccc(-c4ccc5c(c4)c(-c4ccccc4)c(-c4ccccc4)c4ccccc45)cc3)c3ccc(-c4ccccc4)c(-c4ccccc4)c3)c21 ; CC1(c2ccccc2)c2ccccc2-c2ccc(N(c3ccc(-c4ccc5c(c4)c(-c4ccccc4)c(-c4ccccc4)c4ccccc45)cc3)c3ccc(-c4ccccc4)c(-c4ccccc4)c3)cc21 ; c1ccc(-c2ccc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)cc2-c2ccccc2)cc1]

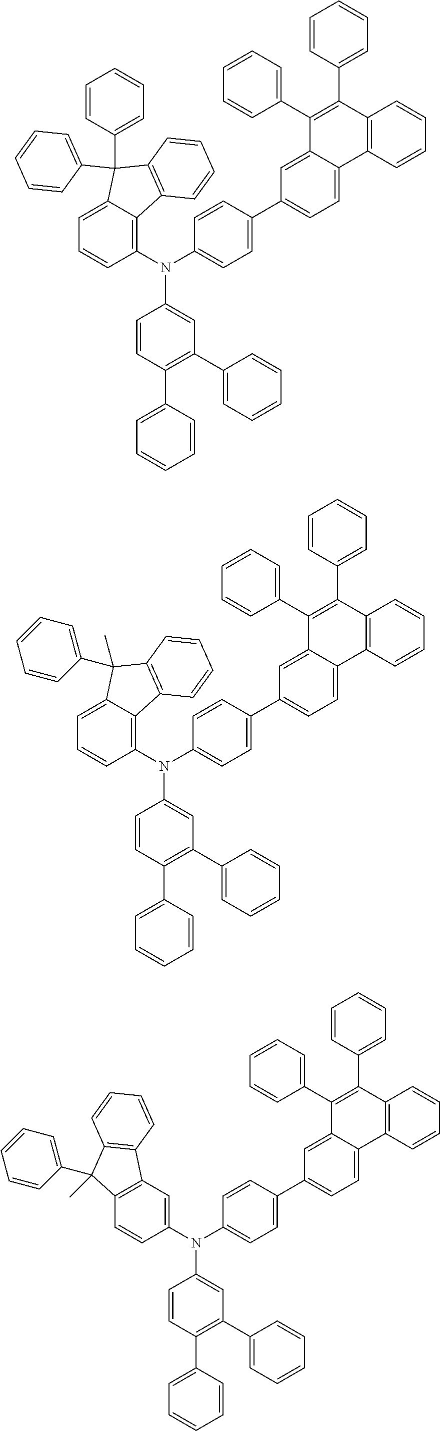 [SMILES: c1ccc(-c2ccc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)cc2-c2ccccc2)cc1 ; CC1(c2ccccc2)c2ccccc2-c2c(N(c3ccc(-c4ccc5c(c4)c(-c4ccccc4)c(-c4ccccc4)c4ccccc45)cc3)c3ccc(-c4ccccc4)c(-c4ccccc4)c3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3ccc(-c4ccccc4)c(-c4ccccc4)c3)ccc21]

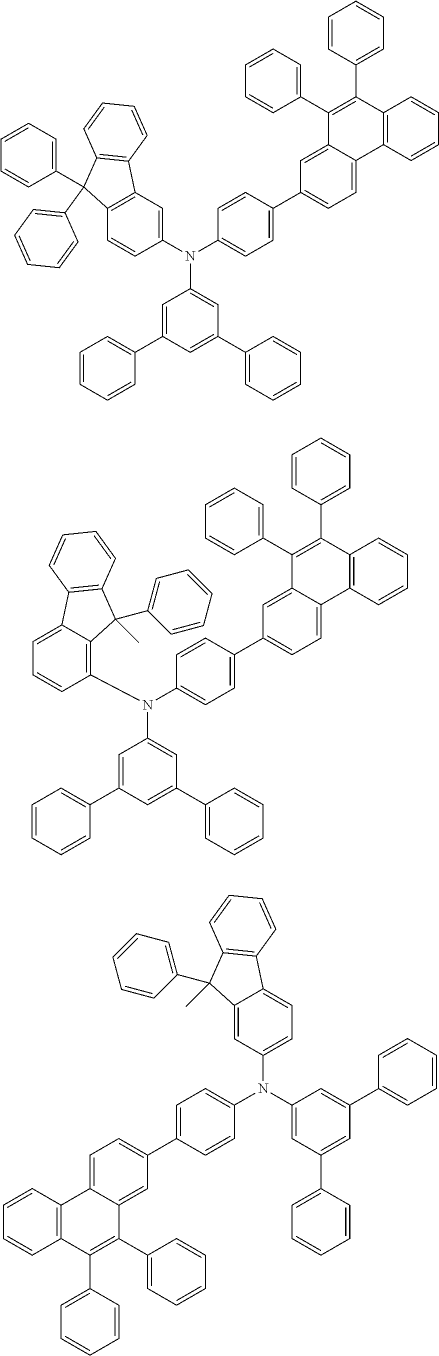

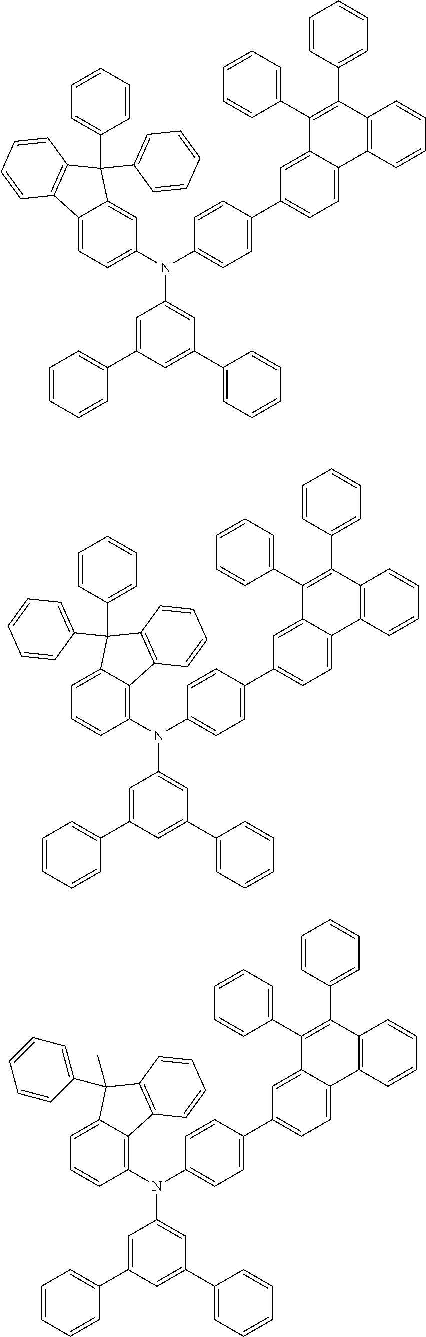 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc4c(c3)C(c3ccccc3)(c3ccccc3)c3ccccc3-4)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cccc4c3-c3ccccc3C4(c3ccccc3)c3ccccc3)c2)cc1 ; CC1(c2ccccc2)c2ccccc2-c2c(N(c3ccc(-c4ccc5c(c4)c(-c4ccccc4)c(-c4ccccc4)c4ccccc45)cc3)c3cc(-c4ccccc4)cc(-c4ccccc4)c3)cccc21]

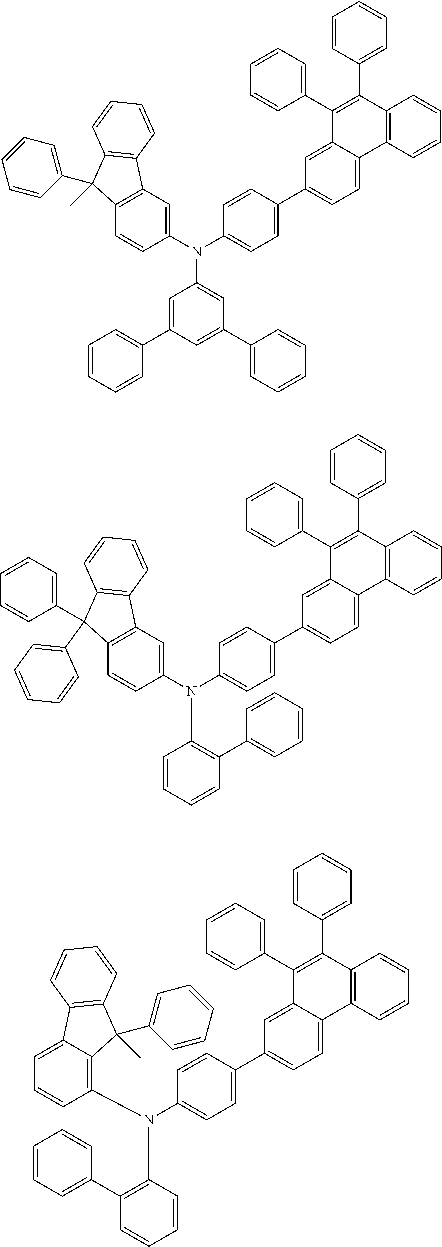 [SMILES: CC1(c2ccccc2)c2ccccc2-c2cc(N(c3ccc(-c4ccc5c(c4)c(-c4ccccc4)c(-c4ccccc4)c4ccccc45)cc3)c3cc(-c4ccccc4)cc(-c4ccccc4)c3)ccc21 ; c1ccc(-c2ccccc2N(c2ccc(-c3ccc4c(c3)c(-c3ccccc3)c(-c3ccccc3)c3ccccc34)cc2)c2ccc3c(c2)-c2ccccc2C3(c2ccccc2)c2ccccc2)cc1 ; CC1(c2ccccc2)c2ccccc2-c2cccc(N(c3ccc(-c4ccc5c(c4)c(-c4ccccc4)c(-c4ccccc4)c4ccccc45)cc3)c3ccccc3-c3ccccc3)c21]

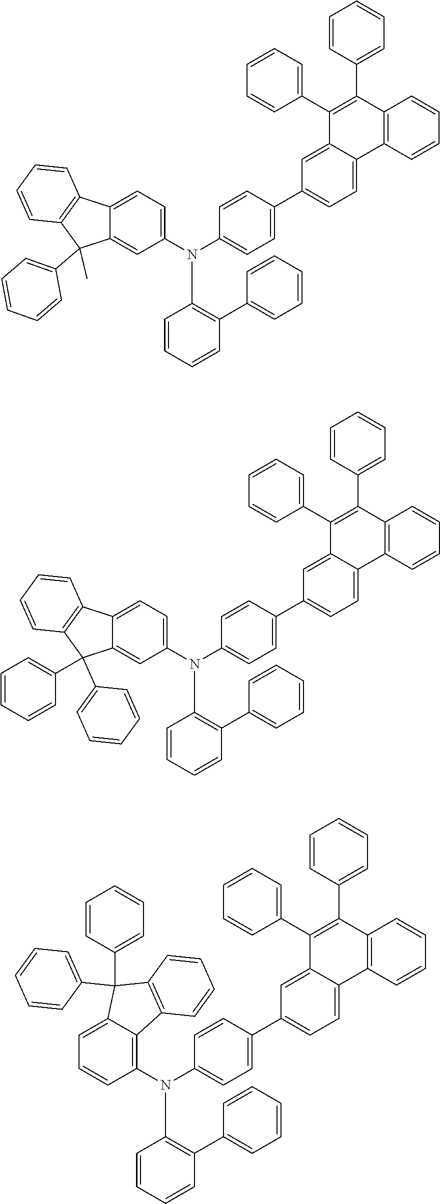 [SMILES: CC1(c2ccccc2)c2ccccc2-c2ccc(N(c3ccc(-c4ccc5c(c4)c(-c4ccccc4)c(-c4ccccc4)c4ccccc45)cc3)c3ccccc3-c3ccccc3)cc21 ; c1ccc(-c2ccccc2N(c2ccc(-c3ccc4c(c3)c(-c3ccccc3)c(-c3ccccc3)c3ccccc34)cc2)c2ccc3c(c2)C(c2ccccc2)(c2ccccc2)c2ccccc2-3)cc1 ; c1ccc(-c2ccccc2N(c2ccc(-c3ccc4c(c3)c(-c3ccccc3)c(-c3ccccc3)c3ccccc34)cc2)c2cccc3c2-c2ccccc2C3(c2ccccc2)c2ccccc2)cc1]

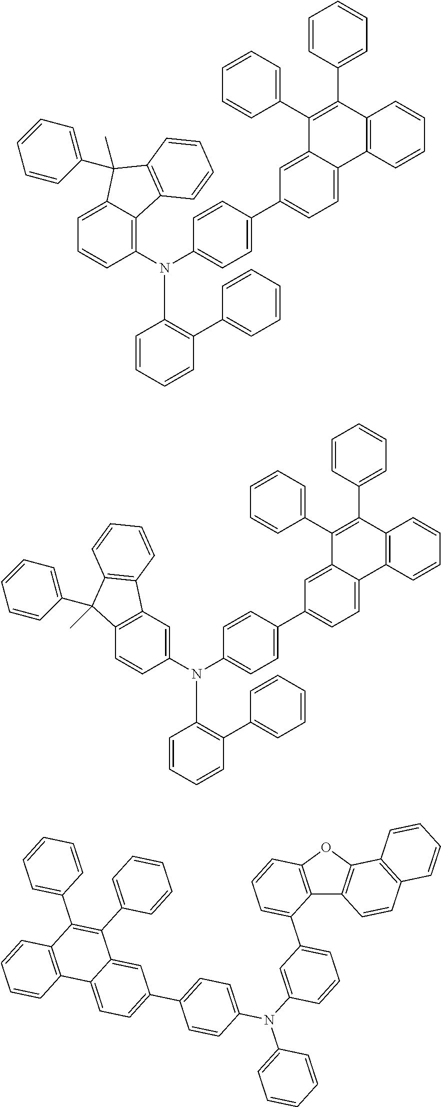 [SMILES: CC1(c2ccccc2)c2ccccc2-c2c(N(c3ccc(-c4ccc5c(c4)c(-c4ccccc4)c(-c4ccccc4)c4ccccc45)cc3)c3ccccc3-c3ccccc3)cccc21 ; CC1(c2ccccc2)c2ccccc2-c2cc(N(c3ccc(-c4ccc5c(c4)c(-c4ccccc4)c(-c4ccccc4)c4ccccc45)cc3)c3ccccc3-c3ccccc3)ccc21 ; c1ccc(-c2c(-c3ccccc3)c3cc(-c4ccc(N(c5ccccc5)c5cccc(-c6cccc7oc8c9ccccc9ccc8c67)c5)cc4)ccc3c3ccccc23)cc1]

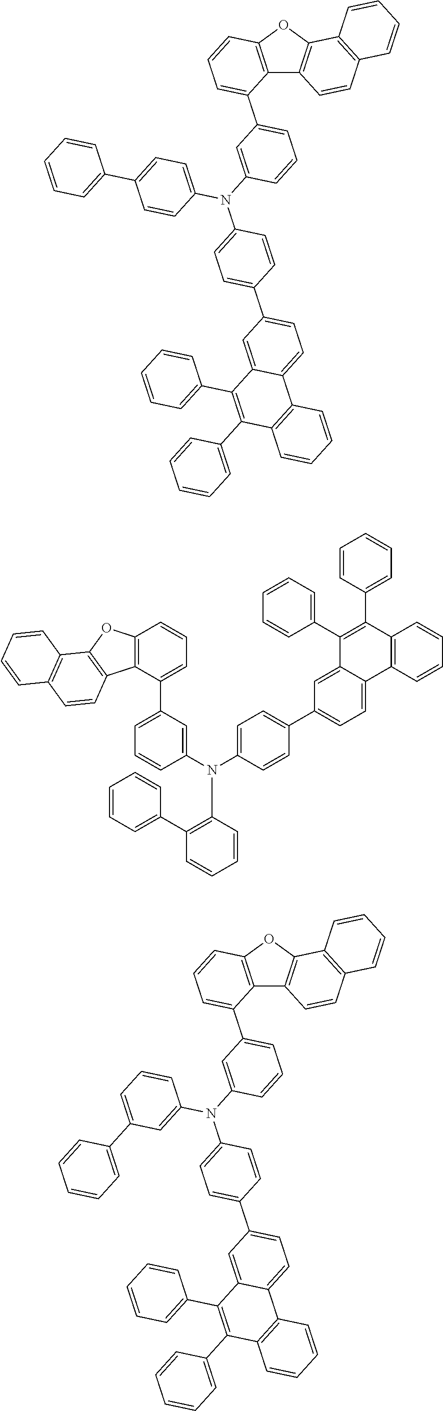 [SMILES: c1ccc(-c2ccc(N(c3ccc(-c4ccc5c(c4)c(-c4ccccc4)c(-c4ccccc4)c4ccccc45)cc3)c3cccc(-c4cccc5oc6c7ccccc7ccc6c45)c3)cc2)cc1 ; c1ccc(-c2ccccc2N(c2ccc(-c3ccc4c(c3)c(-c3ccccc3)c(-c3ccccc3)c3ccccc34)cc2)c2cccc(-c3cccc4oc5c6ccccc6ccc5c34)c2)cc1 ; c1ccc(-c2cccc(N(c3ccc(-c4ccc5c(c4)c(-c4ccccc4)c(-c4ccccc4)c4ccccc45)cc3)c3cccc(-c4cccc5oc6c7ccccc7ccc6c45)c3)c2)cc1]

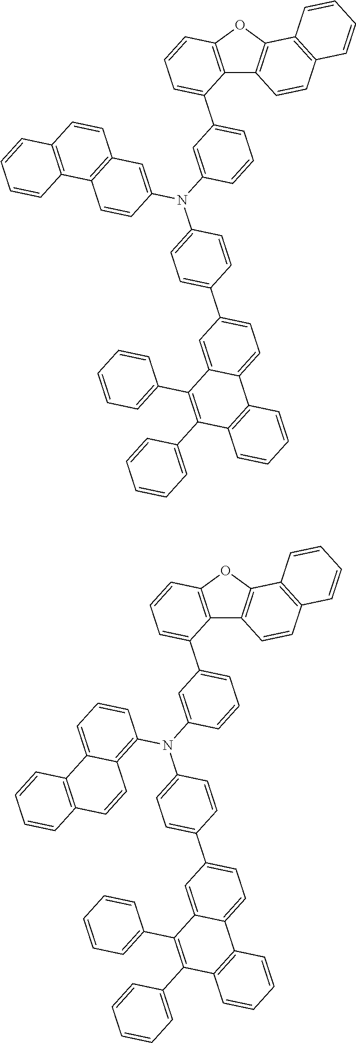 [SMILES: c1ccc(-c2c(-c3ccccc3)c3cc(-c4ccc(N(c5cccc(-c6cccc7oc8c9ccccc9ccc8c67)c5)c5ccc6c(ccc7ccccc76)c5)cc4)ccc3c3ccccc23)cc1 ; c1ccc(-c2c(-c3ccccc3)c3cc(-c4ccc(N(c5cccc(-c6cccc7oc8c9ccccc9ccc8c67)c5)c5cccc6c5ccc5ccccc56)cc4)ccc3c3ccccc23)cc1]

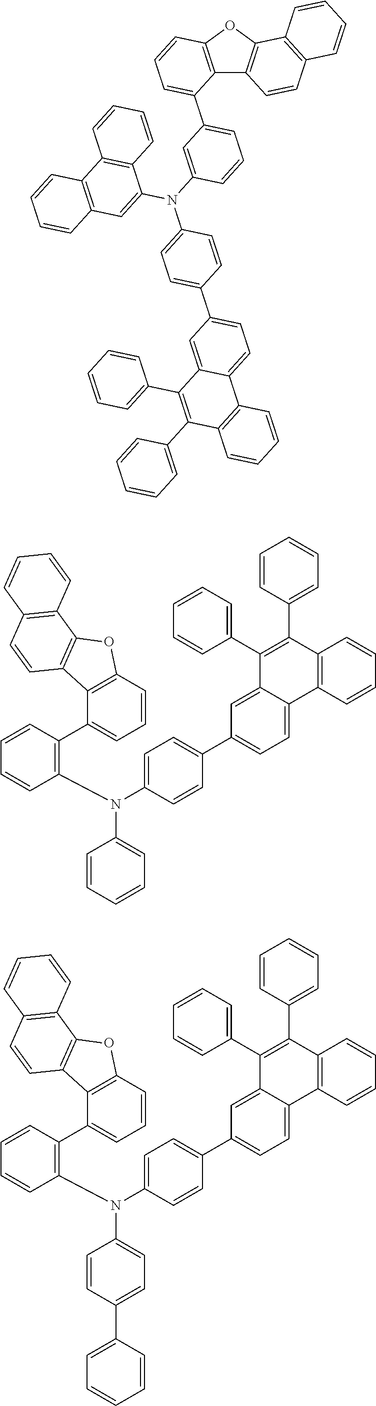 [SMILES: c1ccc(-c2c(-c3ccccc3)c3cc(-c4ccc(N(c5cccc(-c6cccc7oc8c9ccccc9ccc8c67)c5)c5cc6ccccc6c6ccccc56)cc4)ccc3c3ccccc23)cc1 ; c1ccc(-c2c(-c3ccccc3)c3cc(-c4ccc(N(c5ccccc5)c5ccccc5-c5cccc6oc7c8ccccc8ccc7c56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccccc3-c3cccc4oc5c6ccccc6ccc5c34)cc2)cc1]

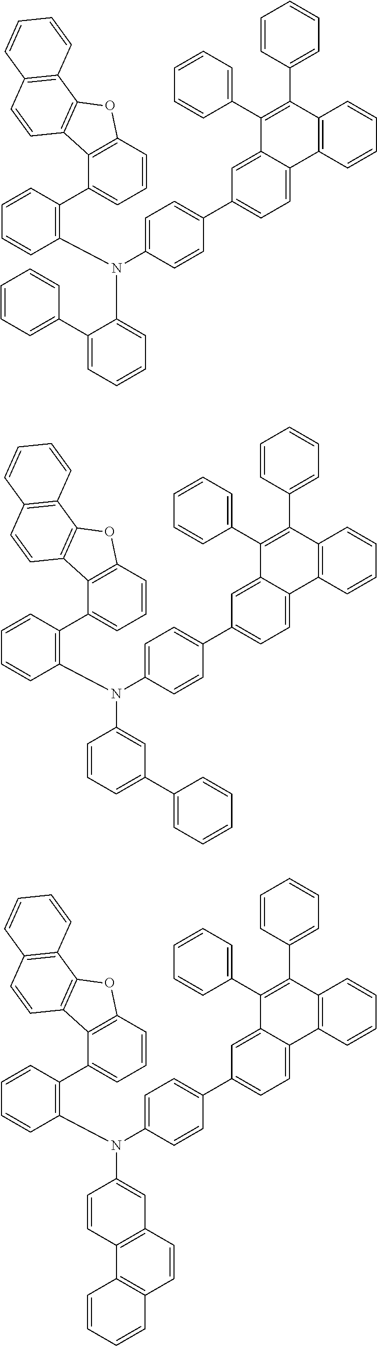 [SMILES: c1ccc(-c2ccccc2N(c2ccc(-c3ccc4c(c3)c(-c3ccccc3)c(-c3ccccc3)c3ccccc34)cc2)c2ccccc2-c2cccc3oc4c5ccccc5ccc4c23)cc1 ; c1ccc(-c2cccc(N(c3ccc(-c4ccc5c(c4)c(-c4ccccc4)c(-c4ccccc4)c4ccccc45)cc3)c3ccccc3-c3cccc4oc5c6ccccc6ccc5c34)c2)cc1 ; c1ccc(-c2c(-c3ccccc3)c3cc(-c4ccc(N(c5ccc6c(ccc7ccccc76)c5)c5ccccc5-c5cccc6oc7c8ccccc8ccc7c56)cc4)ccc3c3ccccc23)cc1]

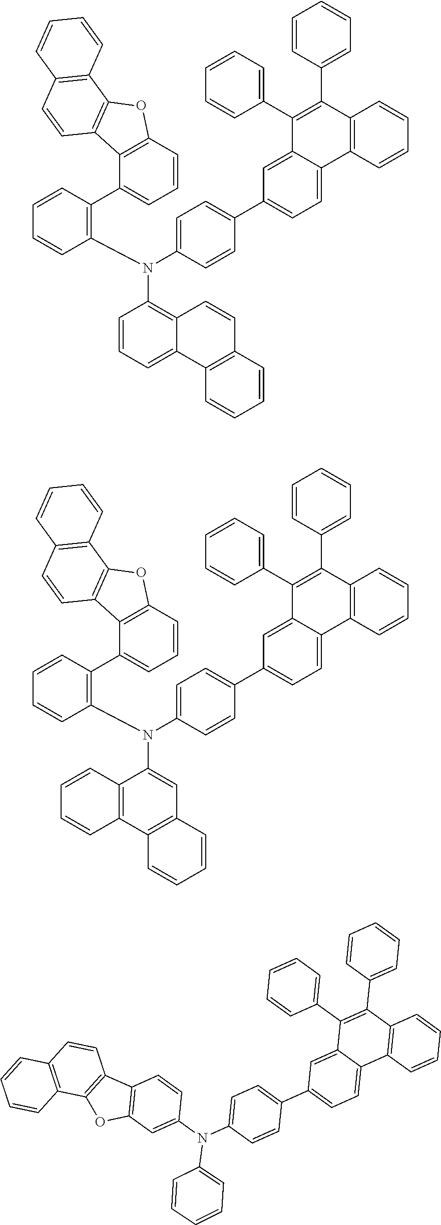 [SMILES: c1ccc(-c2c(-c3ccccc3)c3cc(-c4ccc(N(c5ccccc5-c5cccc6oc7c8ccccc8ccc7c56)c5cccc6c5ccc5ccccc56)cc4)ccc3c3ccccc23)cc1 ; c1ccc(-c2c(-c3ccccc3)c3cc(-c4ccc(N(c5ccccc5-c5cccc6oc7c8ccccc8ccc7c56)c5cc6ccccc6c6ccccc56)cc4)ccc3c3ccccc23)cc1 ; c1ccc(-c2c(-c3ccccc3)c3cc(-c4ccc(N(c5ccccc5)c5ccc6c(c5)oc5c7ccccc7ccc65)cc4)ccc3c3ccccc23)cc1]

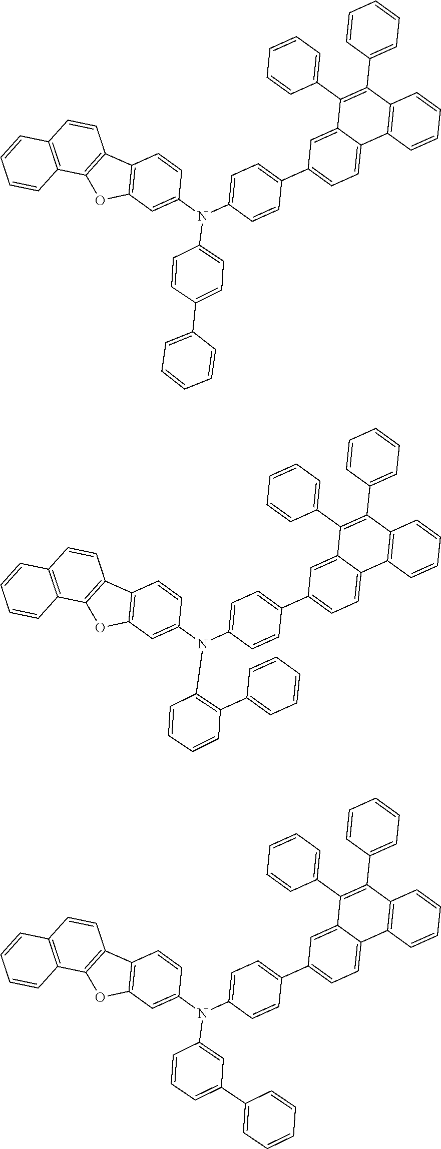 [SMILES: c1ccc(-c2ccc(N(c3ccc(-c4ccc5c(c4)c(-c4ccccc4)c(-c4ccccc4)c4ccccc45)cc3)c3ccc4c(c3)oc3c5ccccc5ccc43)cc2)cc1 ; c1ccc(-c2ccccc2N(c2ccc(-c3ccc4c(c3)c(-c3ccccc3)c(-c3ccccc3)c3ccccc34)cc2)c2ccc3c(c2)oc2c4ccccc4ccc32)cc1 ; c1ccc(-c2cccc(N(c3ccc(-c4ccc5c(c4)c(-c4ccccc4)c(-c4ccccc4)c4ccccc45)cc3)c3ccc4c(c3)oc3c5ccccc5ccc43)c2)cc1]

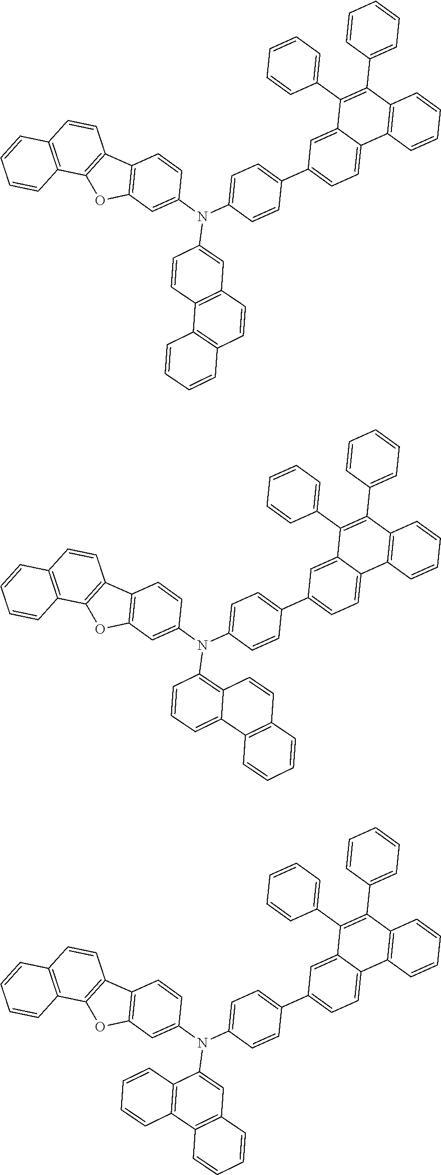 [SMILES: c1ccc(-c2c(-c3ccccc3)c3cc(-c4ccc(N(c5ccc6c(ccc7ccccc76)c5)c5ccc6c(c5)oc5c7ccccc7ccc65)cc4)ccc3c3ccccc23)cc1 ; c1ccc(-c2c(-c3ccccc3)c3cc(-c4ccc(N(c5ccc6c(c5)oc5c7ccccc7ccc65)c5cccc6c5ccc5ccccc56)cc4)ccc3c3ccccc23)cc1 ; c1ccc(-c2c(-c3ccccc3)c3cc(-c4ccc(N(c5ccc6c(c5)oc5c7ccccc7ccc65)c5cc6ccccc6c6ccccc56)cc4)ccc3c3ccccc23)cc1]

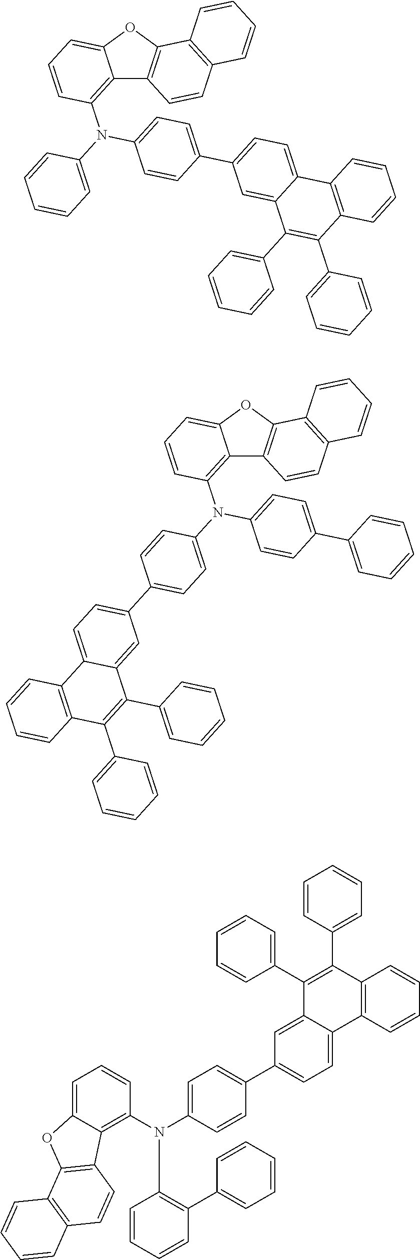

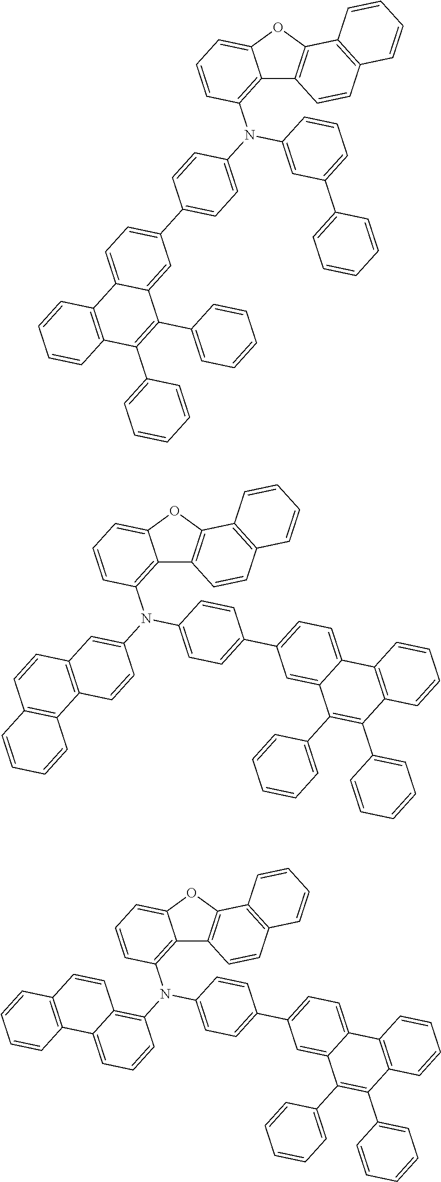 [SMILES: c1ccc(-c2cccc(N(c3ccc(-c4ccc5c(c4)c(-c4ccccc4)c(-c4ccccc4)c4ccccc45)cc3)c3cccc4oc5c6ccccc6ccc5c34)c2)cc1 ; c1ccc(-c2c(-c3ccccc3)c3cc(-c4ccc(N(c5ccc6c(ccc7ccccc76)c5)c5cccc6oc7c8ccccc8ccc7c56)cc4)ccc3c3ccccc23)cc1 ; c1ccc(-c2c(-c3ccccc3)c3cc(-c4ccc(N(c5cccc6c5ccc5ccccc56)c5cccc6oc7c8ccccc8ccc7c56)cc4)ccc3c3ccccc23)cc1]

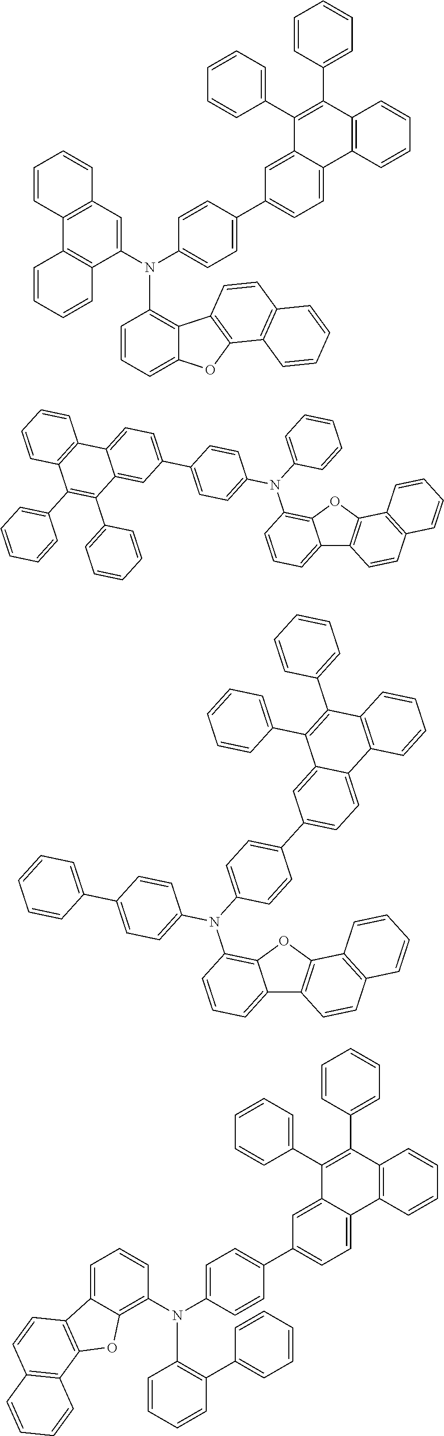

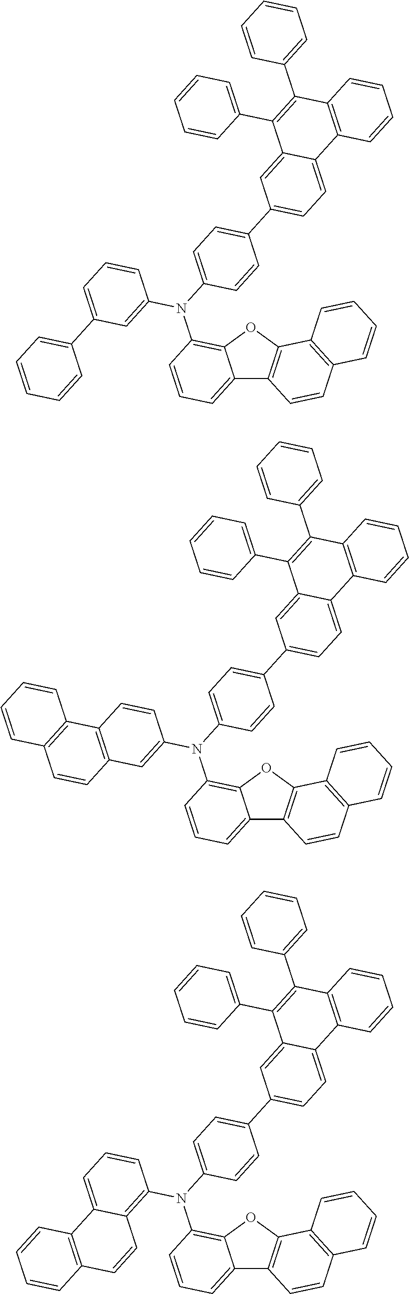

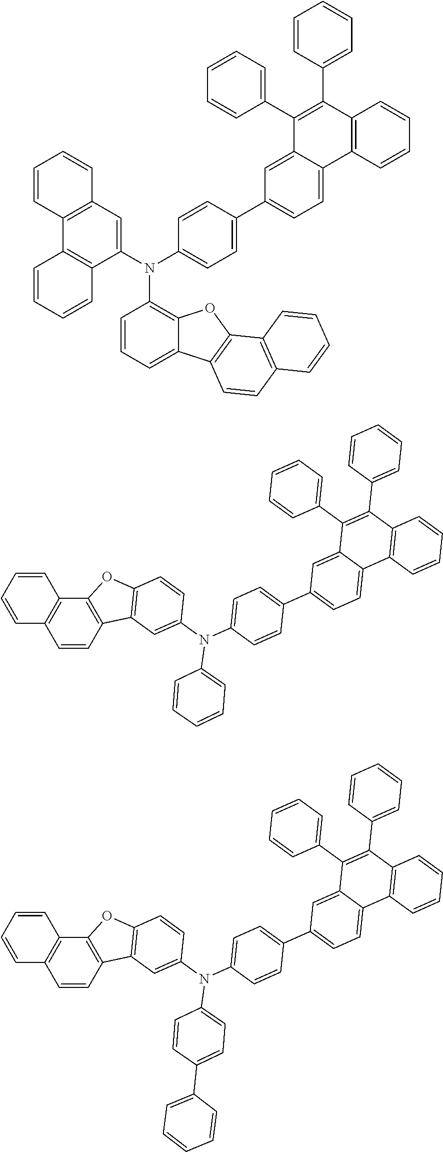 [SMILES: c1ccc(-c2c(-c3ccccc3)c3cc(-c4ccc(N(c5cc6ccccc6c6ccccc56)c5cccc6c5oc5c7ccccc7ccc65)cc4)ccc3c3ccccc23)cc1 ; c1ccc(-c2c(-c3ccccc3)c3cc(-c4ccc(N(c5ccccc5)c5ccc6oc7c8ccccc8ccc7c6c5)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3ccc4oc5c6ccccc6ccc5c4c3)cc2)cc1]

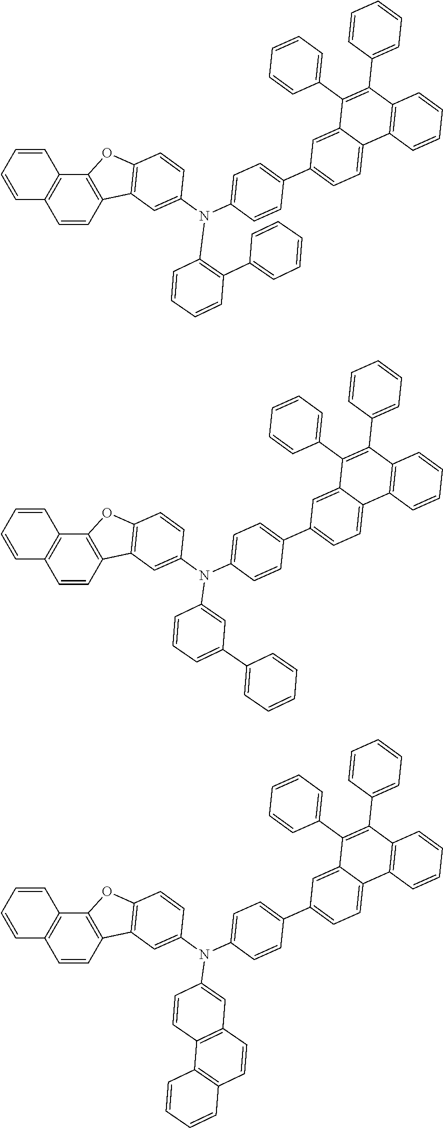 [SMILES: c1ccc(-c2ccccc2N(c2ccc(-c3ccc4c(c3)c(-c3ccccc3)c(-c3ccccc3)c3ccccc34)cc2)c2ccc3oc4c5ccccc5ccc4c3c2)cc1 ; c1ccc(-c2cccc(N(c3ccc(-c4ccc5c(c4)c(-c4ccccc4)c(-c4ccccc4)c4ccccc45)cc3)c3ccc4oc5c6ccccc6ccc5c4c3)c2)cc1 ; c1ccc(-c2c(-c3ccccc3)c3cc(-c4ccc(N(c5ccc6c(ccc7ccccc76)c5)c5ccc6oc7c8ccccc8ccc7c6c5)cc4)ccc3c3ccccc23)cc1]

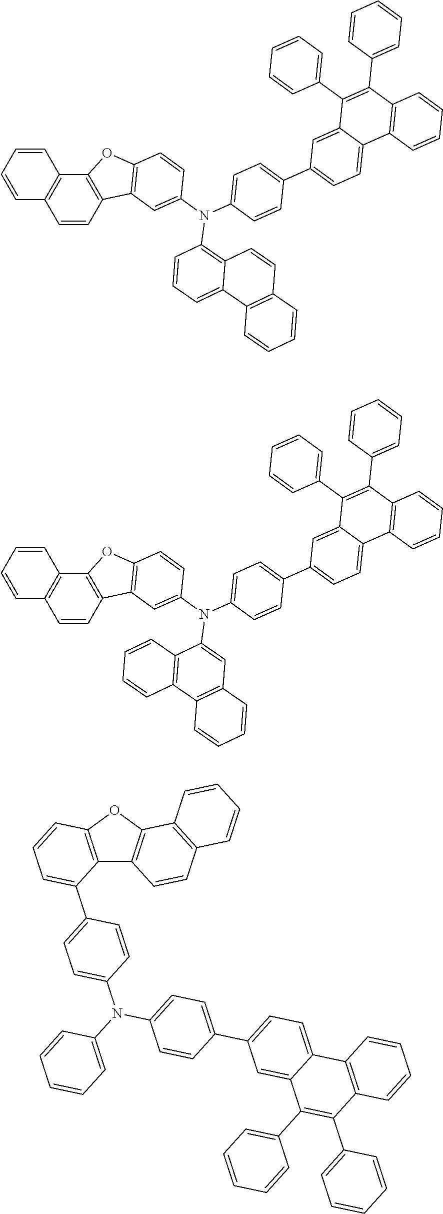

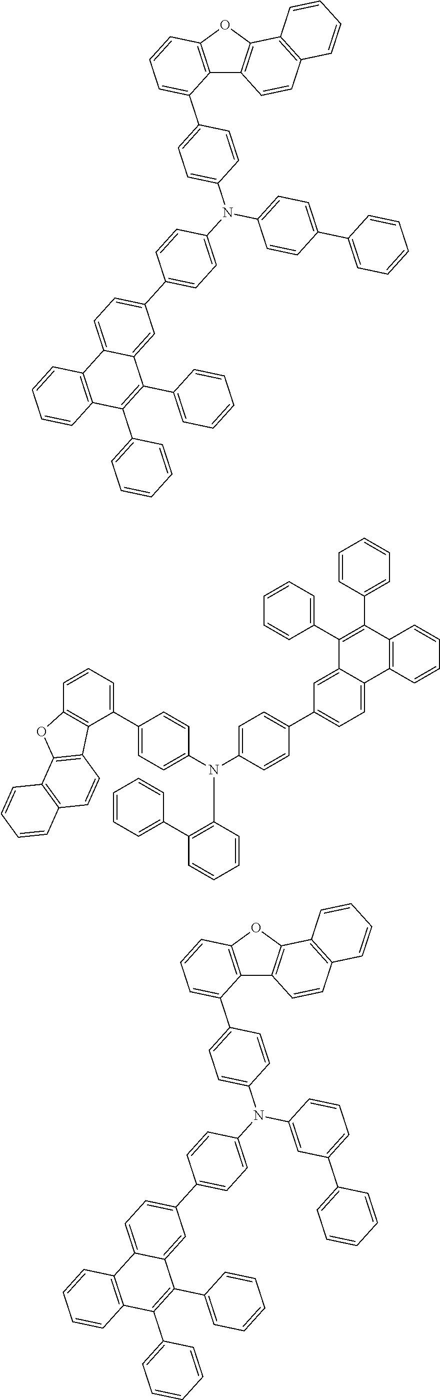 [SMILES: c1ccc(-c2ccc(N(c3ccc(-c4ccc5c(c4)c(-c4ccccc4)c(-c4ccccc4)c4ccccc45)cc3)c3ccc(-c4cccc5oc6c7ccccc7ccc6c45)cc3)cc2)cc1 ; c1ccc(-c2ccccc2N(c2ccc(-c3ccc4c(c3)c(-c3ccccc3)c(-c3ccccc3)c3ccccc34)cc2)c2ccc(-c3cccc4oc5c6ccccc6ccc5c34)cc2)cc1 ; c1ccc(-c2cccc(N(c3ccc(-c4ccc5c(c4)c(-c4ccccc4)c(-c4ccccc4)c4ccccc45)cc3)c3ccc(-c4cccc5oc6c7ccccc7ccc6c45)cc3)c2)cc1]

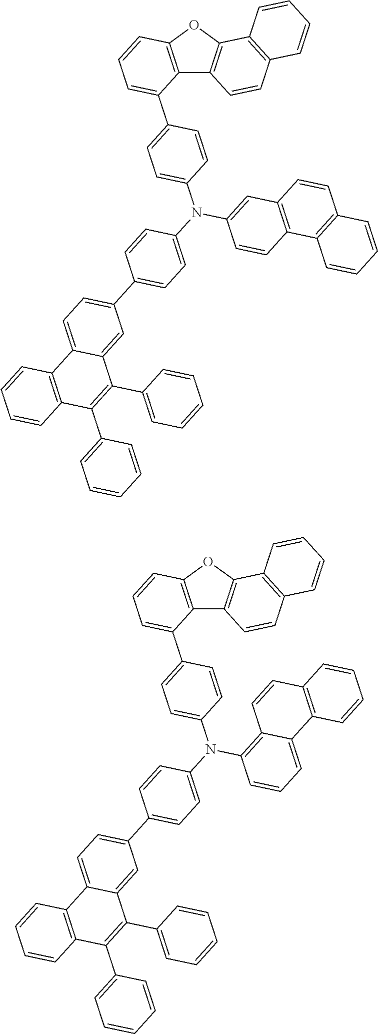 [SMILES: c1ccc(-c2c(-c3ccccc3)c3cc(-c4ccc(N(c5ccc(-c6cccc7oc8c9ccccc9ccc8c67)cc5)c5ccc6c(ccc7ccccc76)c5)cc4)ccc3c3ccccc23)cc1 ; c1ccc(-c2c(-c3ccccc3)c3cc(-c4ccc(N(c5ccc(-c6cccc7oc8c9ccccc9ccc8c67)cc5)c5cccc6c5ccc5ccccc56)cc4)ccc3c3ccccc23)cc1]

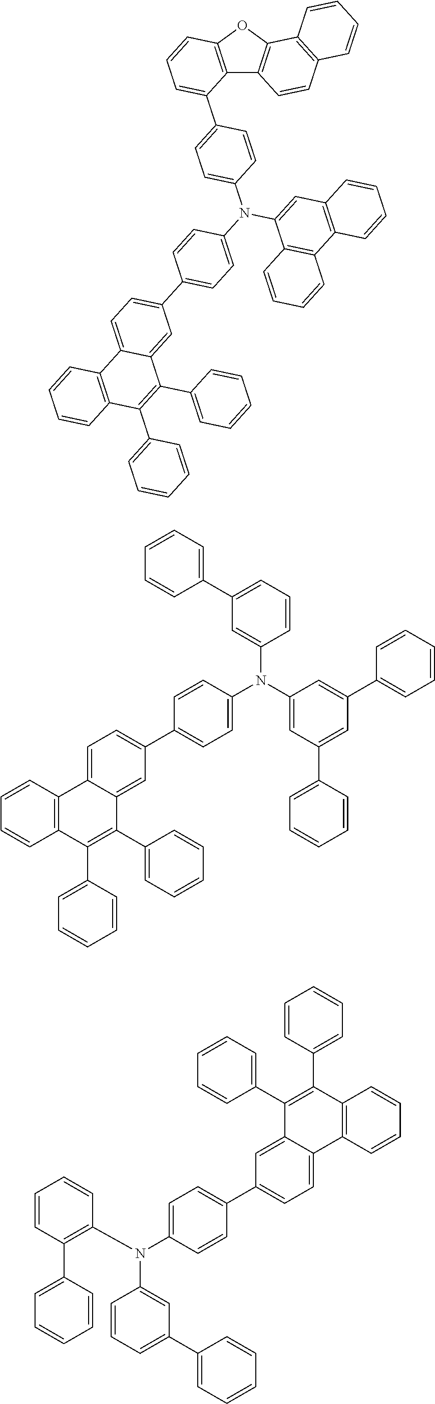

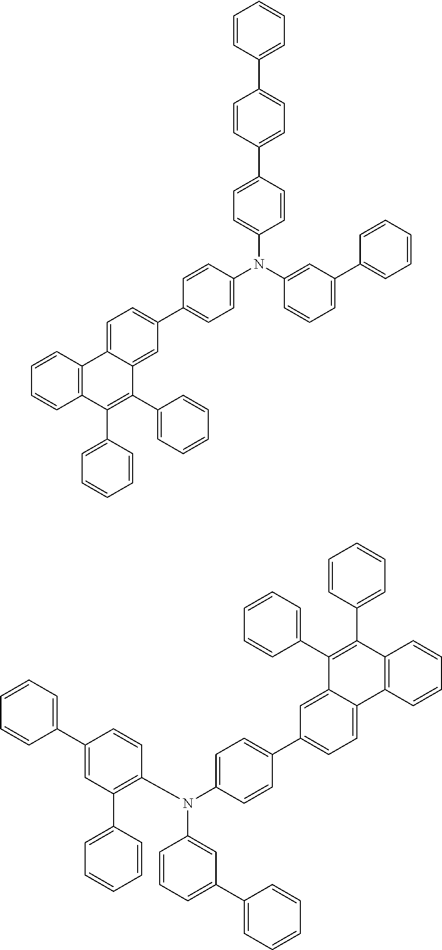 [SMILES: c1ccc(-c2ccc(-c3ccc(N(c4ccc(-c5ccc6c(c5)c(-c5ccccc5)c(-c5ccccc5)c5ccccc56)cc4)c4cccc(-c5ccccc5)c4)cc3)cc2)cc1 ; c1ccc(-c2cccc(N(c3ccc(-c4ccc5c(c4)c(-c4ccccc4)c(-c4ccccc4)c4ccccc45)cc3)c3ccc(-c4ccccc4)cc3-c3ccccc3)c2)cc1]

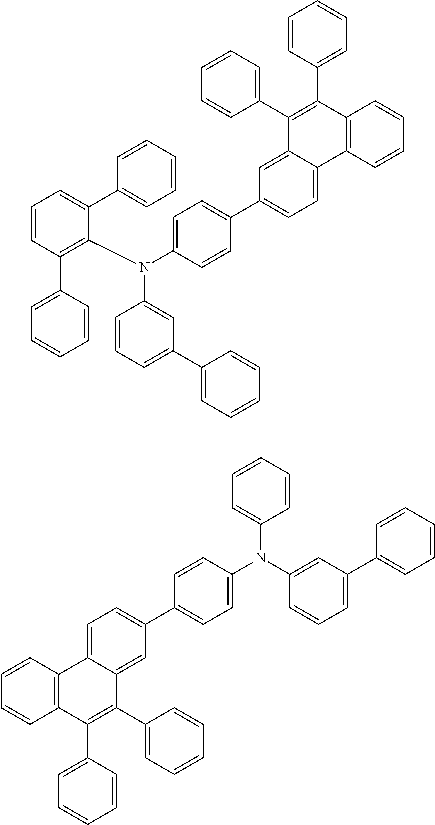 [SMILES: c1ccc(-c2cccc(N(c3ccc(-c4ccc5c(c4)c(-c4ccccc4)c(-c4ccccc4)c4ccccc45)cc3)c3c(-c4ccccc4)cccc3-c3ccccc3)c2)cc1 ; c1ccc(-c2cccc(N(c3ccccc3)c3ccc(-c4ccc5c(c4)c(-c4ccccc4)c(-c4ccccc4)c4ccccc45)cc3)c2)cc1]

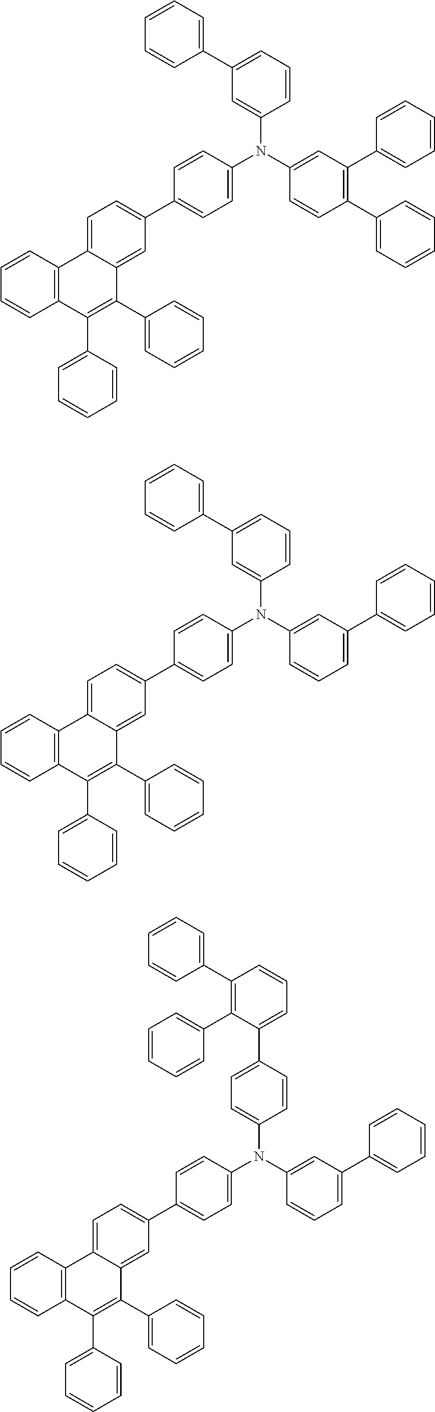 [SMILES: c1ccc(-c2cccc(N(c3ccc(-c4ccc5c(c4)c(-c4ccccc4)c(-c4ccccc4)c4ccccc45)cc3)c3ccc(-c4ccccc4)c(-c4ccccc4)c3)c2)cc1 ; c1ccc(-c2cccc(N(c3ccc(-c4ccc5c(c4)c(-c4ccccc4)c(-c4ccccc4)c4ccccc45)cc3)c3cccc(-c4ccccc4)c3)c2)cc1 ; c1ccc(-c2cccc(N(c3ccc(-c4ccc5c(c4)c(-c4ccccc4)c(-c4ccccc4)c4ccccc45)cc3)c3ccc(-c4cccc(-c5ccccc5)c4-c4ccccc4)cc3)c2)cc1]

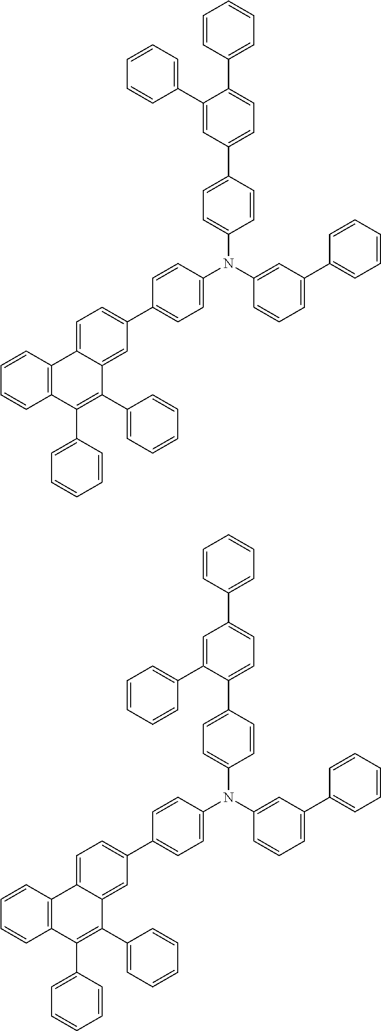 [SMILES: c1ccc(-c2cccc(N(c3ccc(-c4ccc(-c5ccccc5)c(-c5ccccc5)c4)cc3)c3ccc(-c4ccc5c(c4)c(-c4ccccc4)c(-c4ccccc4)c4ccccc45)cc3)c2)cc1 ; c1ccc(-c2cccc(N(c3ccc(-c4ccc5c(c4)c(-c4ccccc4)c(-c4ccccc4)c4ccccc45)cc3)c3ccc(-c4ccc(-c5ccccc5)cc4-c4ccccc4)cc3)c2)cc1]

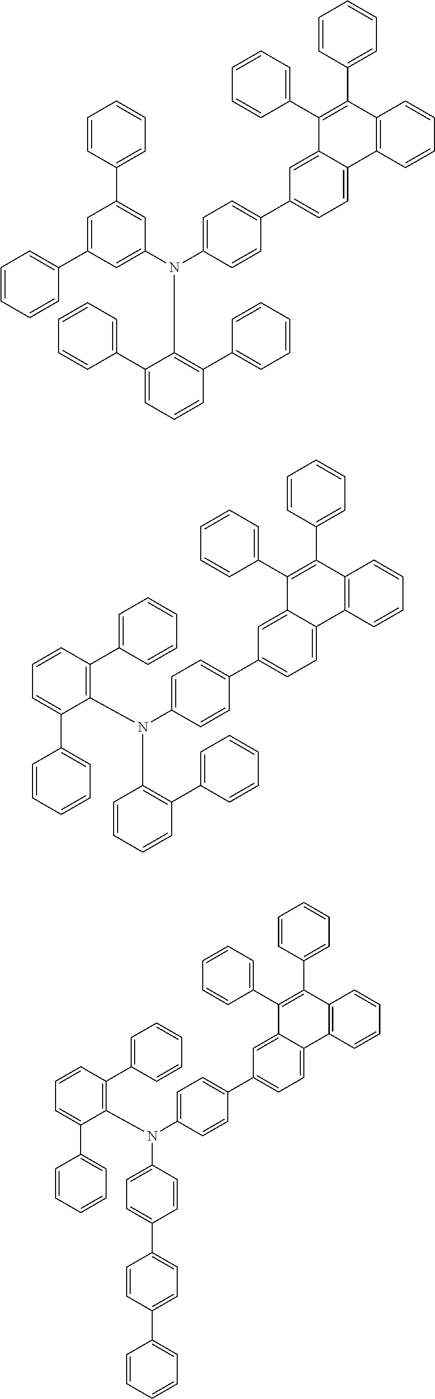 [SMILES: c1ccc(-c2cc(-c3ccccc3)cc(N(c3ccc(-c4ccc5c(c4)c(-c4ccccc4)c(-c4ccccc4)c4ccccc45)cc3)c3c(-c4ccccc4)cccc3-c3ccccc3)c2)cc1 ; c1ccc(-c2ccccc2N(c2ccc(-c3ccc4c(c3)c(-c3ccccc3)c(-c3ccccc3)c3ccccc34)cc2)c2c(-c3ccccc3)cccc2-c2ccccc2)cc1 ; c1ccc(-c2ccc(-c3ccc(N(c4ccc(-c5ccc6c(c5)c(-c5ccccc5)c(-c5ccccc5)c5ccccc56)cc4)c4c(-c5ccccc5)cccc4-c4ccccc4)cc3)cc2)cc1]

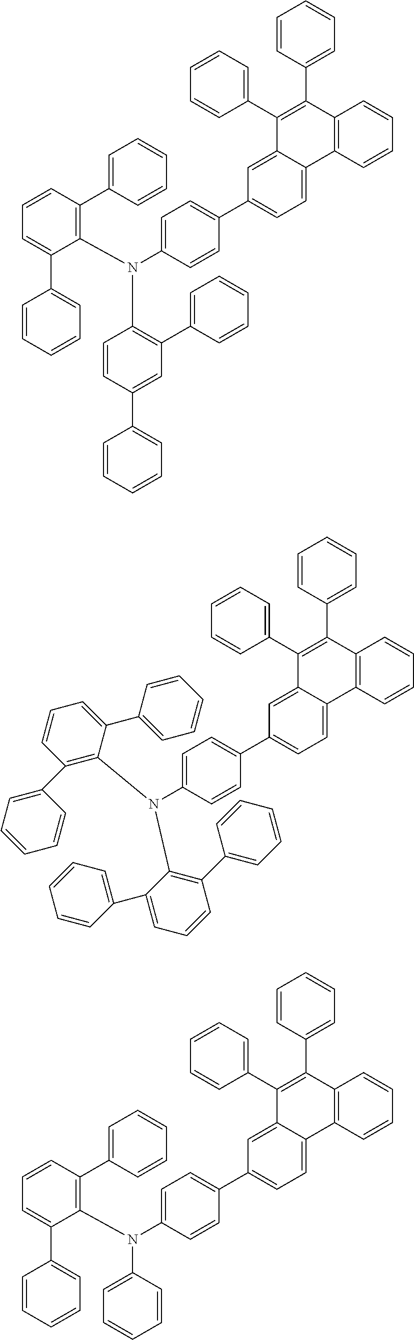 [SMILES: c1ccc(-c2ccc(N(c3ccc(-c4ccc5c(c4)c(-c4ccccc4)c(-c4ccccc4)c4ccccc45)cc3)c3c(-c4ccccc4)cccc3-c3ccccc3)c(-c3ccccc3)c2)cc1 ; c1ccc(-c2cccc(-c3ccccc3)c2N(c2ccc(-c3ccc4c(c3)c(-c3ccccc3)c(-c3ccccc3)c3ccccc34)cc2)c2c(-c3ccccc3)cccc2-c2ccccc2)cc1 ; c1ccc(-c2cccc(-c3ccccc3)c2N(c2ccccc2)c2ccc(-c3ccc4c(c3)c(-c3ccccc3)c(-c3ccccc3)c3ccccc34)cc2)cc1]

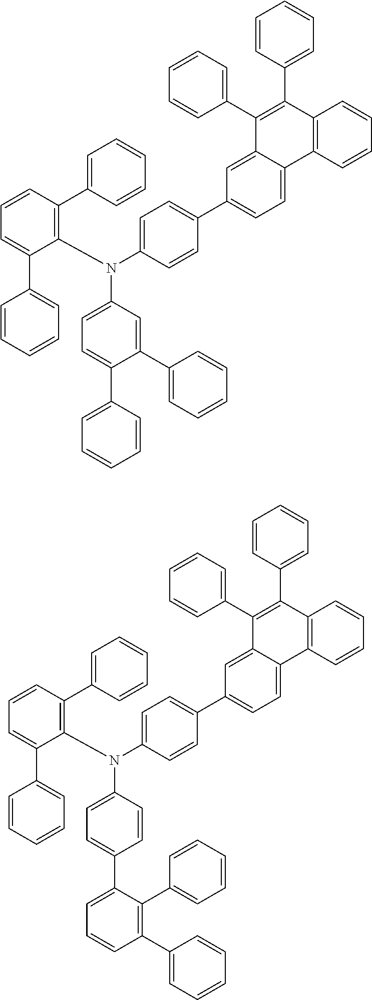 [SMILES: c1ccc(-c2ccc(N(c3ccc(-c4ccc5c(c4)c(-c4ccccc4)c(-c4ccccc4)c4ccccc45)cc3)c3c(-c4ccccc4)cccc3-c3ccccc3)cc2-c2ccccc2)cc1 ; c1ccc(-c2cccc(-c3ccc(N(c4ccc(-c5ccc6c(c5)c(-c5ccccc5)c(-c5ccccc5)c5ccccc56)cc4)c4c(-c5ccccc5)cccc4-c4ccccc4)cc3)c2-c2ccccc2)cc1]

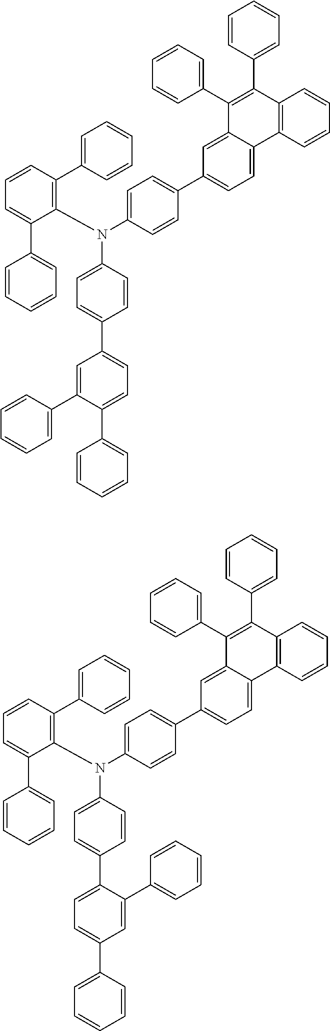 [SMILES: c1ccc(-c2ccc(-c3ccc(N(c4ccc(-c5ccc6c(c5)c(-c5ccccc5)c(-c5ccccc5)c5ccccc56)cc4)c4c(-c5ccccc5)cccc4-c4ccccc4)cc3)cc2-c2ccccc2)cc1 ; c1ccc(-c2ccc(-c3ccc(N(c4ccc(-c5ccc6c(c5)c(-c5ccccc5)c(-c5ccccc5)c5ccccc56)cc4)c4c(-c5ccccc5)cccc4-c4ccccc4)cc3)c(-c3ccccc3)c2)cc1]

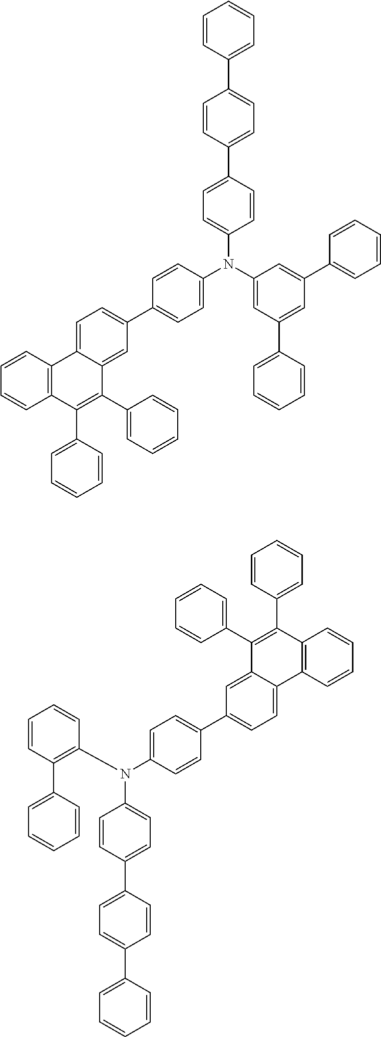 [SMILES: c1ccc(-c2ccc(-c3ccc(N(c4ccc(-c5ccc6c(c5)c(-c5ccccc5)c(-c5ccccc5)c5ccccc56)cc4)c4cc(-c5ccccc5)cc(-c5ccccc5)c4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccccc4-c4ccccc4)cc3)cc2)cc1]

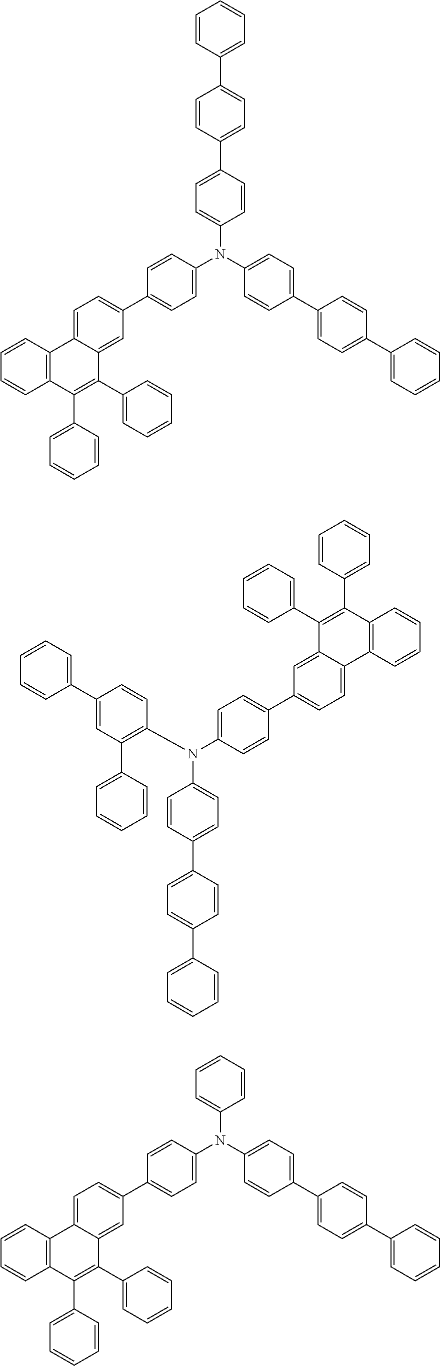 [SMILES: c1ccc(-c2ccc(-c3ccc(N(c4ccc(-c5ccc(-c6ccccc6)cc5)cc4)c4ccc(-c5ccc6c(c5)c(-c5ccccc5)c(-c5ccccc5)c5ccccc56)cc4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5ccccc5)cc4-c4ccccc4)cc3)cc2)cc1 ; c1ccc(-c2ccc(-c3ccc(N(c4ccccc4)c4ccc(-c5ccc6c(c5)c(-c5ccccc5)c(-c5ccccc5)c5ccccc56)cc4)cc3)cc2)cc1]

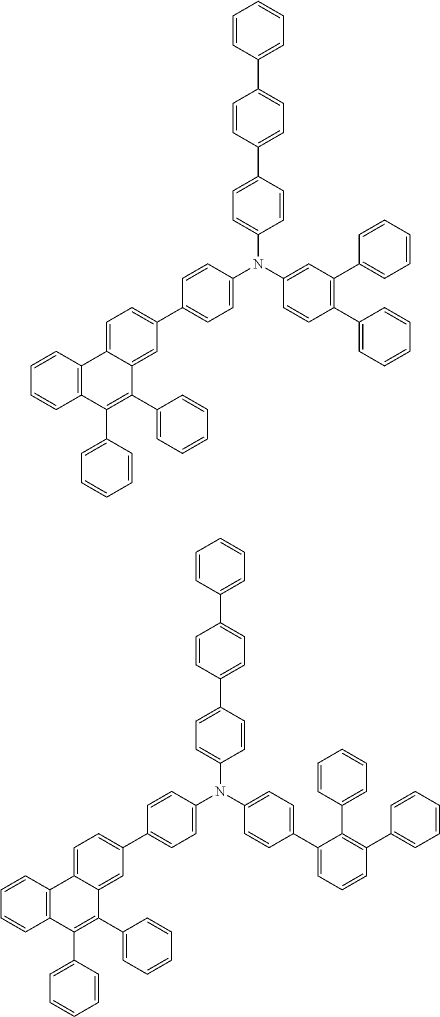 [SMILES: c1ccc(-c2ccc(-c3ccc(N(c4ccc(-c5ccc6c(c5)c(-c5ccccc5)c(-c5ccccc5)c5ccccc56)cc4)c4ccc(-c5ccccc5)c(-c5ccccc5)c4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5cccc(-c6ccccc6)c5-c5ccccc5)cc4)cc3)cc2)cc1]

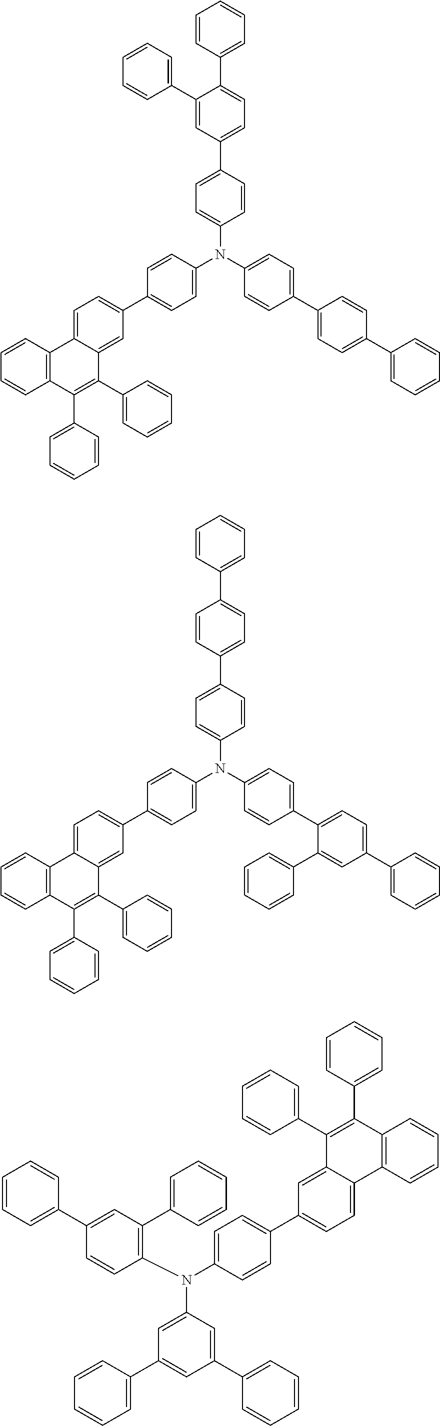 [SMILES: c1ccc(-c2ccc(-c3ccc(N(c4ccc(-c5ccc(-c6ccccc6)c(-c6ccccc6)c5)cc4)c4ccc(-c5ccc6c(c5)c(-c5ccccc5)c(-c5ccccc5)c5ccccc56)cc4)cc3)cc2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5ccc(-c6ccccc6)cc5-c5ccccc5)cc4)cc3)cc2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc(-c4ccccc4)cc3-c3ccccc3)c2)cc1]

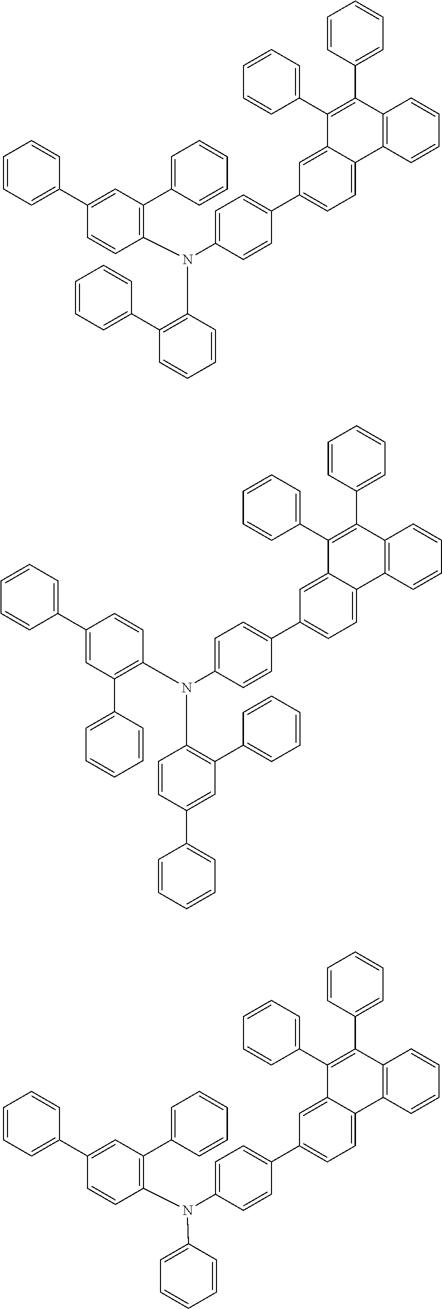 [SMILES: c1ccc(-c2ccc(N(c3ccc(-c4ccc5c(c4)c(-c4ccccc4)c(-c4ccccc4)c4ccccc45)cc3)c3ccccc3-c3ccccc3)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc(-c4ccccc4)cc3-c3ccccc3)c(-c3ccccc3)c2)cc1 ; c1ccc(-c2ccc(N(c3ccccc3)c3ccc(-c4ccc5c(c4)c(-c4ccccc4)c(-c4ccccc4)c4ccccc45)cc3)c(-c3ccccc3)c2)cc1]

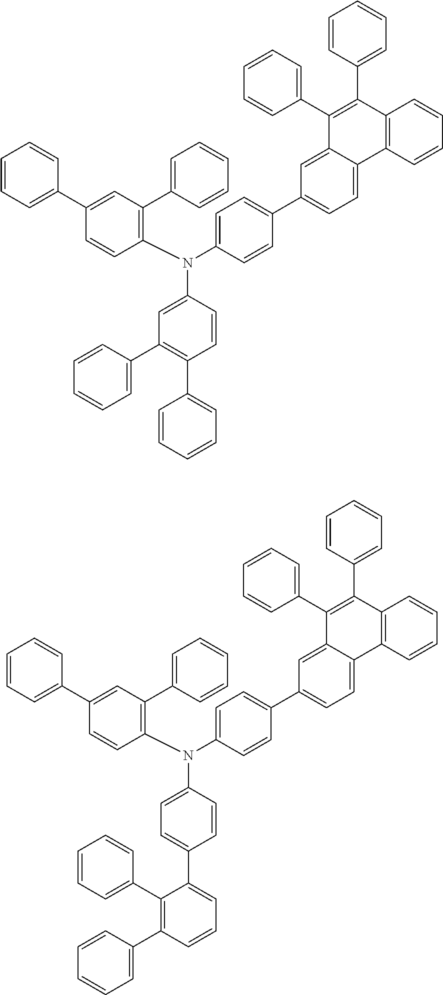 [SMILES: c1ccc(-c2ccc(N(c3ccc(-c4ccc5c(c4)c(-c4ccccc4)c(-c4ccccc4)c4ccccc45)cc3)c3ccc(-c4ccccc4)c(-c4ccccc4)c3)c(-c3ccccc3)c2)cc1 ; c1ccc(-c2ccc(N(c3ccc(-c4ccc5c(c4)c(-c4ccccc4)c(-c4ccccc4)c4ccccc45)cc3)c3ccc(-c4cccc(-c5ccccc5)c4-c4ccccc4)cc3)c(-c3ccccc3)c2)cc1]

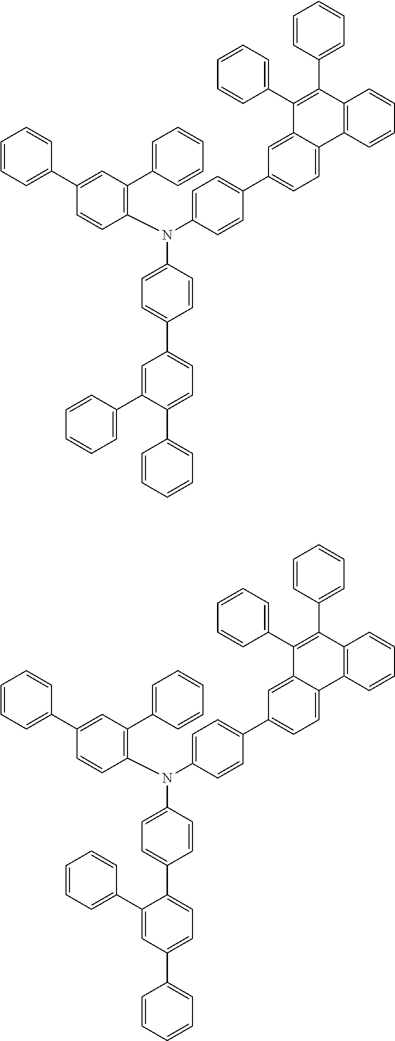 [SMILES: c1ccc(-c2ccc(N(c3ccc(-c4ccc(-c5ccccc5)c(-c5ccccc5)c4)cc3)c3ccc(-c4ccc5c(c4)c(-c4ccccc4)c(-c4ccccc4)c4ccccc45)cc3)c(-c3ccccc3)c2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5ccccc5)cc4-c4ccccc4)cc3)c(-c3ccccc3)c2)cc1]

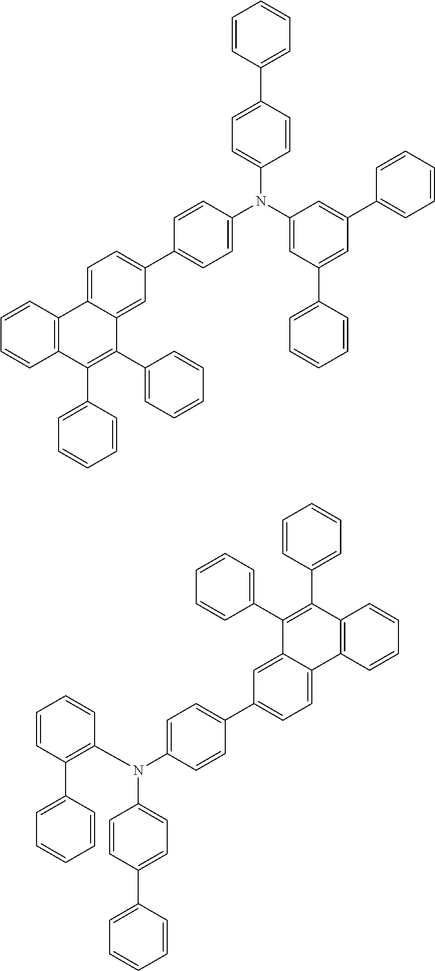 [SMILES: c1ccc(-c2ccc(N(c3ccc(-c4ccc5c(c4)c(-c4ccccc4)c(-c4ccccc4)c4ccccc45)cc3)c3cc(-c4ccccc4)cc(-c4ccccc4)c3)cc2)cc1 ; c1ccc(-c2ccc(N(c3ccc(-c4ccc5c(c4)c(-c4ccccc4)c(-c4ccccc4)c4ccccc45)cc3)c3ccccc3-c3ccccc3)cc2)cc1]

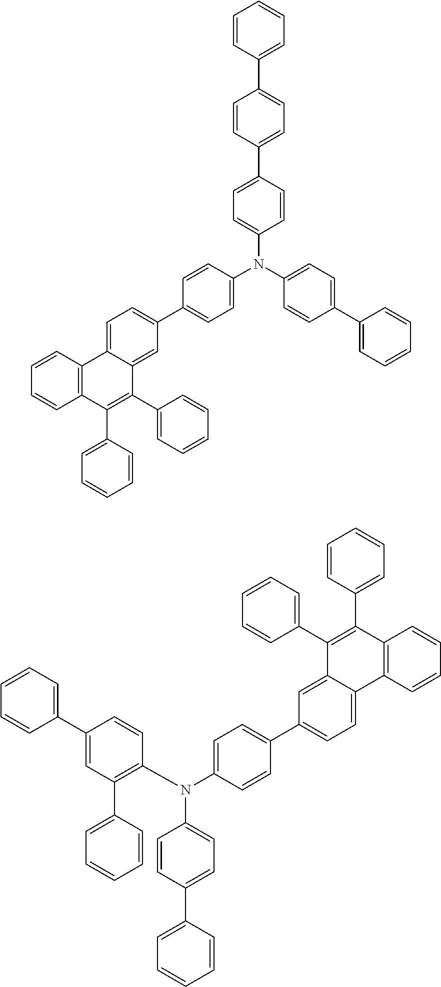 [SMILES: c1ccc(-c2ccc(-c3ccc(N(c4ccc(-c5ccccc5)cc4)c4ccc(-c5ccc6c(c5)c(-c5ccccc5)c(-c5ccccc5)c5ccccc56)cc4)cc3)cc2)cc1 ; c1ccc(-c2ccc(N(c3ccc(-c4ccc5c(c4)c(-c4ccccc4)c(-c4ccccc4)c4ccccc45)cc3)c3ccc(-c4ccccc4)cc3-c3ccccc3)cc2)cc1]

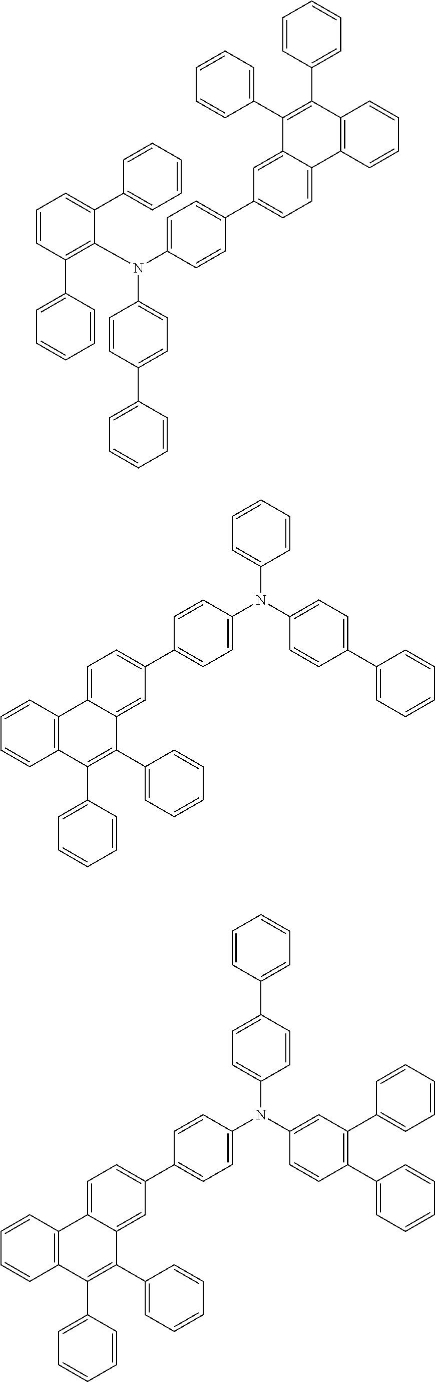 [SMILES: c1ccc(-c2ccc(N(c3ccc(-c4ccc5c(c4)c(-c4ccccc4)c(-c4ccccc4)c4ccccc45)cc3)c3c(-c4ccccc4)cccc3-c3ccccc3)cc2)cc1 ; c1ccc(-c2ccc(N(c3ccccc3)c3ccc(-c4ccc5c(c4)c(-c4ccccc4)c(-c4ccccc4)c4ccccc45)cc3)cc2)cc1 ; c1ccc(-c2ccc(N(c3ccc(-c4ccc5c(c4)c(-c4ccccc4)c(-c4ccccc4)c4ccccc45)cc3)c3ccc(-c4ccccc4)c(-c4ccccc4)c3)cc2)cc1]

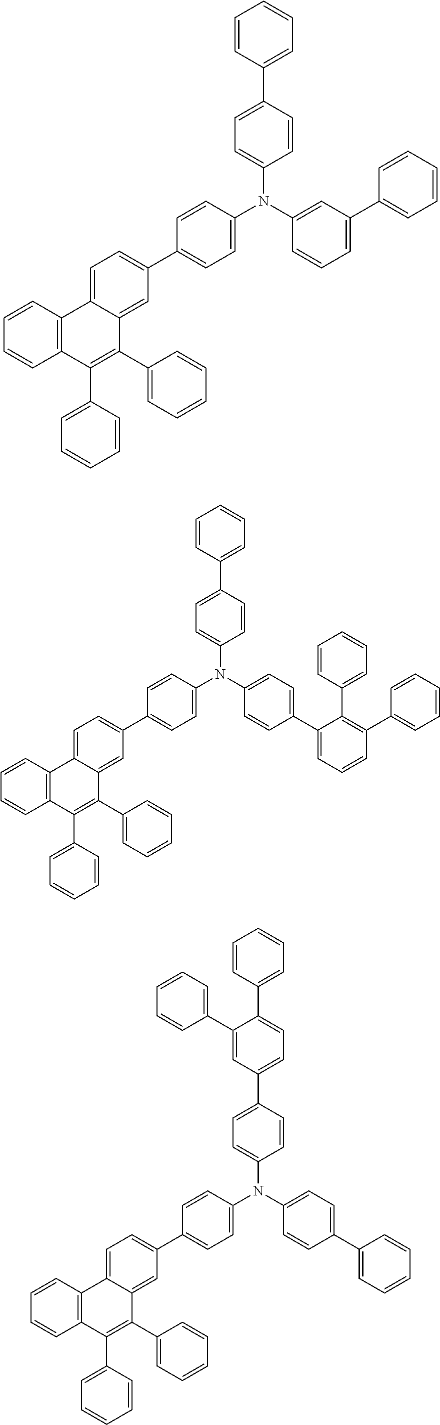 [SMILES: c1ccc(-c2ccc(N(c3ccc(-c4ccc5c(c4)c(-c4ccccc4)c(-c4ccccc4)c4ccccc45)cc3)c3cccc(-c4ccccc4)c3)cc2)cc1 ; c1ccc(-c2ccc(N(c3ccc(-c4ccc5c(c4)c(-c4ccccc4)c(-c4ccccc4)c4ccccc45)cc3)c3ccc(-c4cccc(-c5ccccc5)c4-c4ccccc4)cc3)cc2)cc1 ; c1ccc(-c2ccc(N(c3ccc(-c4ccc(-c5ccccc5)c(-c5ccccc5)c4)cc3)c3ccc(-c4ccc5c(c4)c(-c4ccccc4)c(-c4ccccc4)c4ccccc45)cc3)cc2)cc1]

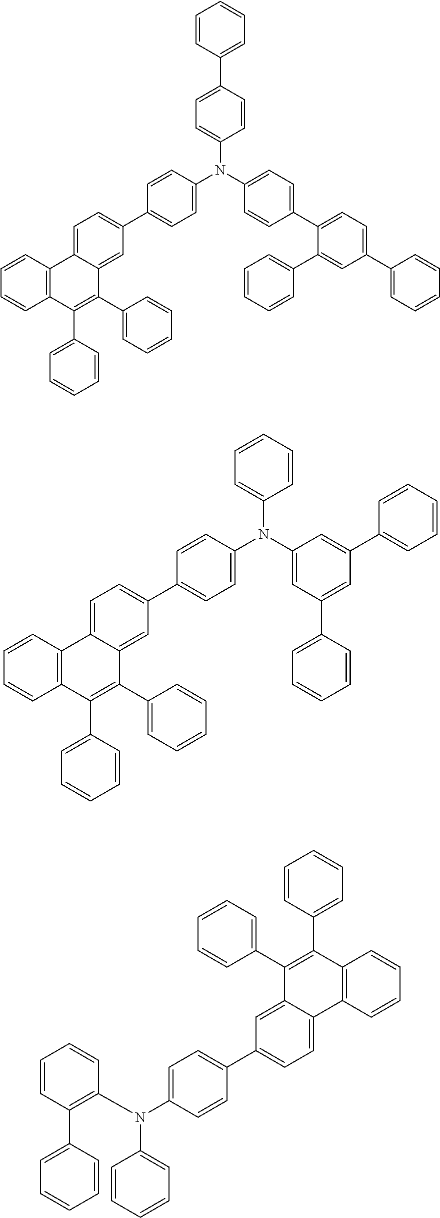 [SMILES: c1ccc(-c2ccc(N(c3ccc(-c4ccc5c(c4)c(-c4ccccc4)c(-c4ccccc4)c4ccccc45)cc3)c3ccc(-c4ccc(-c5ccccc5)cc4-c4ccccc4)cc3)cc2)cc1 ; c1ccc(-c2cc(-c3ccccc3)cc(N(c3ccccc3)c3ccc(-c4ccc5c(c4)c(-c4ccccc4)c(-c4ccccc4)c4ccccc45)cc3)c2)cc1 ; c1ccc(-c2ccccc2N(c2ccccc2)c2ccc(-c3ccc4c(c3)c(-c3ccccc3)c(-c3ccccc3)c3ccccc34)cc2)cc1]

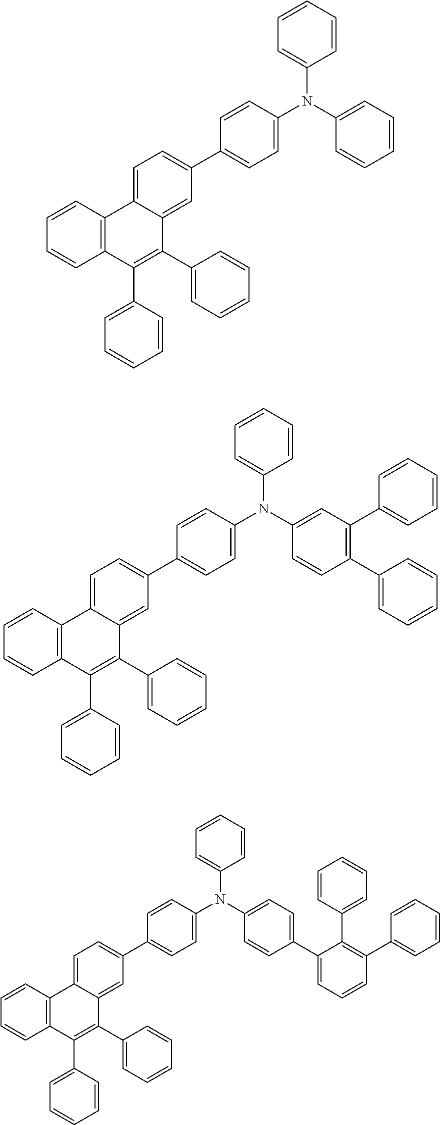 [SMILES: c1ccc(-c2c(-c3ccccc3)c3cc(-c4ccc(N(c5ccccc5)c5ccccc5)cc4)ccc3c3ccccc23)cc1 ; c1ccc(-c2ccc(N(c3ccccc3)c3ccc(-c4ccc5c(c4)c(-c4ccccc4)c(-c4ccccc4)c4ccccc45)cc3)cc2-c2ccccc2)cc1 ; c1ccc(-c2cccc(-c3ccc(N(c4ccccc4)c4ccc(-c5ccc6c(c5)c(-c5ccccc5)c(-c5ccccc5)c5ccccc56)cc4)cc3)c2-c2ccccc2)cc1]

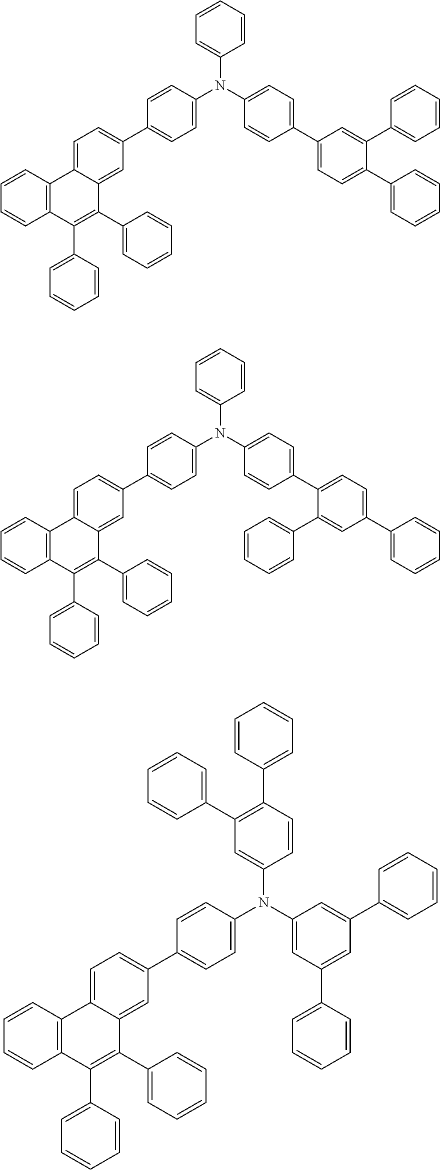 [SMILES: c1ccc(-c2ccc(-c3ccc(N(c4ccccc4)c4ccc(-c5ccc6c(c5)c(-c5ccccc5)c(-c5ccccc5)c5ccccc56)cc4)cc3)cc2-c2ccccc2)cc1 ; c1ccc(-c2ccc(-c3ccc(N(c4ccccc4)c4ccc(-c5ccc6c(c5)c(-c5ccccc5)c(-c5ccccc5)c5ccccc56)cc4)cc3)c(-c3ccccc3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccc(-c4ccccc4)c(-c4ccccc4)c3)c2)cc1]

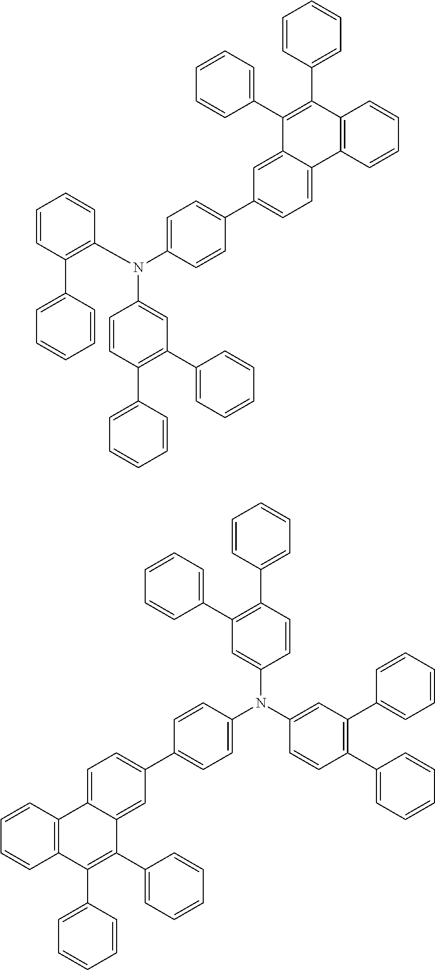 [SMILES: c1ccc(-c2ccc(N(c3ccc(-c4ccc5c(c4)c(-c4ccccc4)c(-c4ccccc4)c4ccccc45)cc3)c3ccccc3-c3ccccc3)cc2-c2ccccc2)cc1 ; c1ccc(-c2ccc(N(c3ccc(-c4ccc5c(c4)c(-c4ccccc4)c(-c4ccccc4)c4ccccc45)cc3)c3ccc(-c4ccccc4)c(-c4ccccc4)c3)cc2-c2ccccc2)cc1]

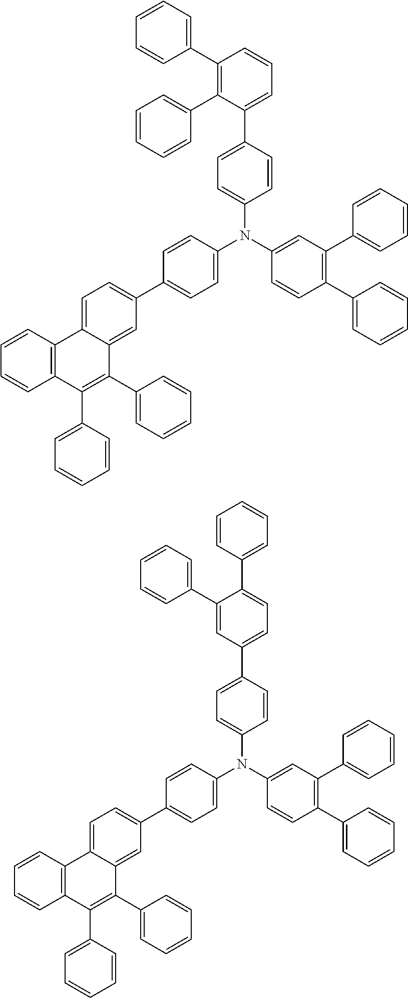 [SMILES: c1ccc(-c2ccc(N(c3ccc(-c4ccc5c(c4)c(-c4ccccc4)c(-c4ccccc4)c4ccccc45)cc3)c3ccc(-c4cccc(-c5ccccc5)c4-c4ccccc4)cc3)cc2-c2ccccc2)cc1 ; c1ccc(-c2ccc(-c3ccc(N(c4ccc(-c5ccc6c(c5)c(-c5ccccc5)c(-c5ccccc5)c5ccccc56)cc4)c4ccc(-c5ccccc5)c(-c5ccccc5)c4)cc3)cc2-c2ccccc2)cc1]

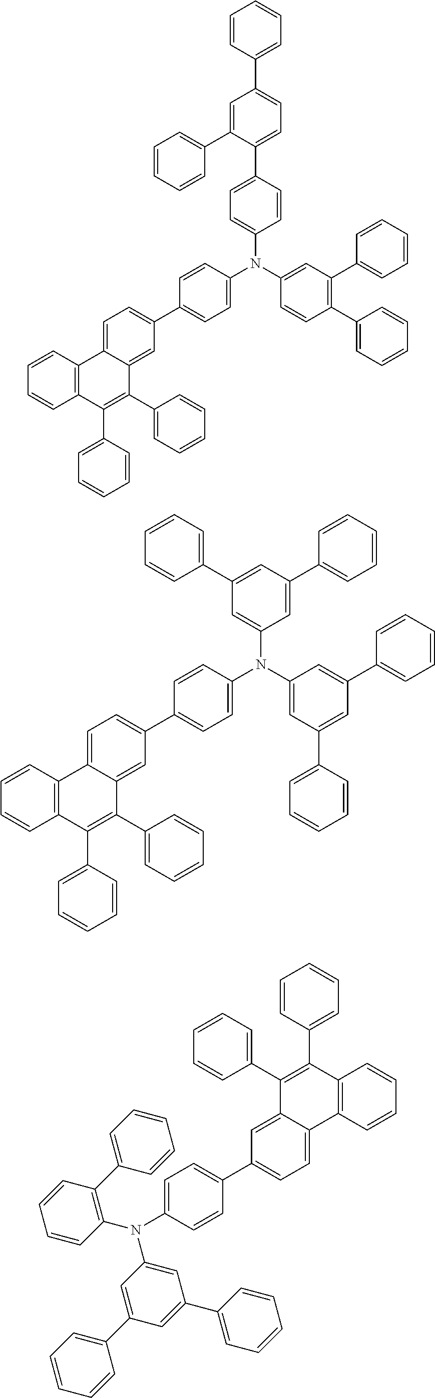 [SMILES: c1ccc(-c2ccc(-c3ccc(N(c4ccc(-c5ccc6c(c5)c(-c5ccccc5)c(-c5ccccc5)c5ccccc56)cc4)c4ccc(-c5ccccc5)c(-c5ccccc5)c4)cc3)c(-c3ccccc3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3cc(-c4ccccc4)cc(-c4ccccc4)c3)c2)cc1 ; c1ccc(-c2cc(-c3ccccc3)cc(N(c3ccc(-c4ccc5c(c4)c(-c4ccccc4)c(-c4ccccc4)c4ccccc45)cc3)c3ccccc3-c3ccccc3)c2)cc1]

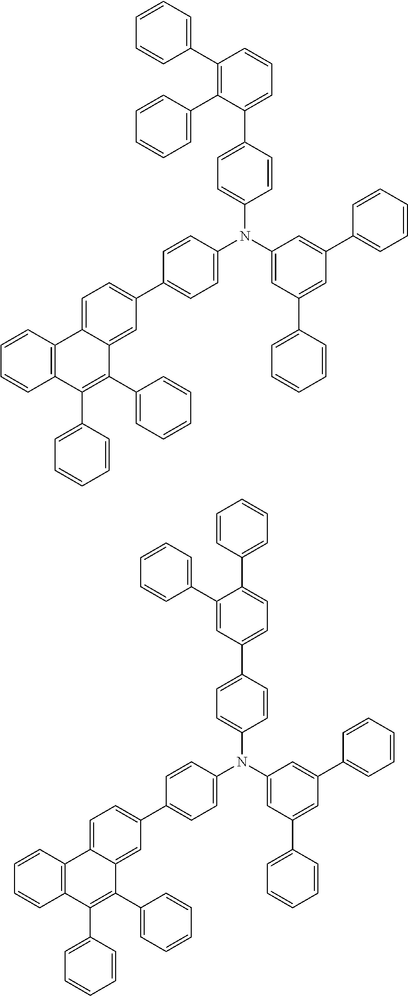 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc(-c4cccc(-c5ccccc5)c4-c4ccccc4)cc3)c2)cc1 ; c1ccc(-c2cc(-c3ccccc3)cc(N(c3ccc(-c4ccc(-c5ccccc5)c(-c5ccccc5)c4)cc3)c3ccc(-c4ccc5c(c4)c(-c4ccccc4)c(-c4ccccc4)c4ccccc45)cc3)c2)cc1]

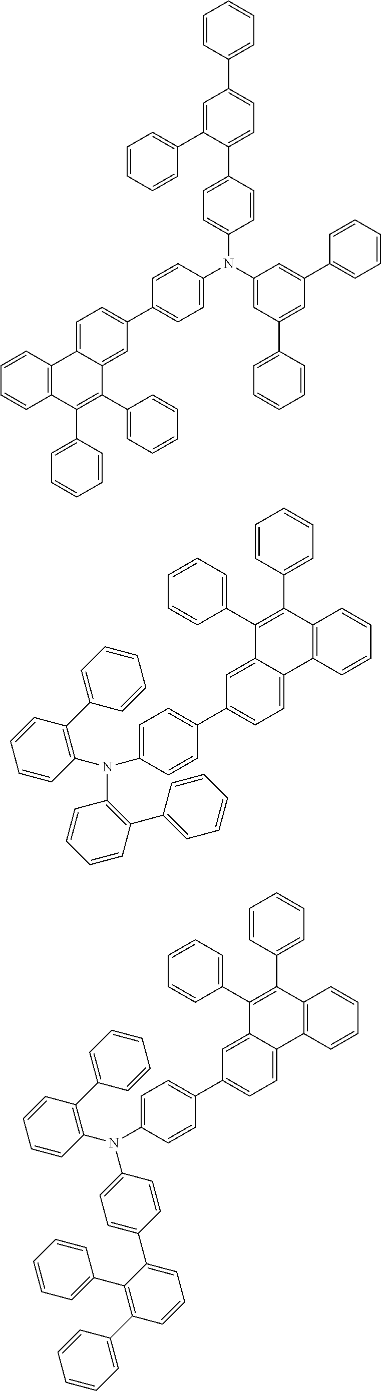 [SMILES: c1ccc(-c2cc(-c3ccccc3)cc(N(c3ccc(-c4ccc5c(c4)c(-c4ccccc4)c(-c4ccccc4)c4ccccc45)cc3)c3ccc(-c4ccc(-c5ccccc5)cc4-c4ccccc4)cc3)c2)cc1 ; c1ccc(-c2ccccc2N(c2ccc(-c3ccc4c(c3)c(-c3ccccc3)c(-c3ccccc3)c3ccccc34)cc2)c2ccccc2-c2ccccc2)cc1 ; c1ccc(-c2ccccc2N(c2ccc(-c3ccc4c(c3)c(-c3ccccc3)c(-c3ccccc3)c3ccccc34)cc2)c2ccc(-c3cccc(-c4ccccc4)c3-c3ccccc3)cc2)cc1]

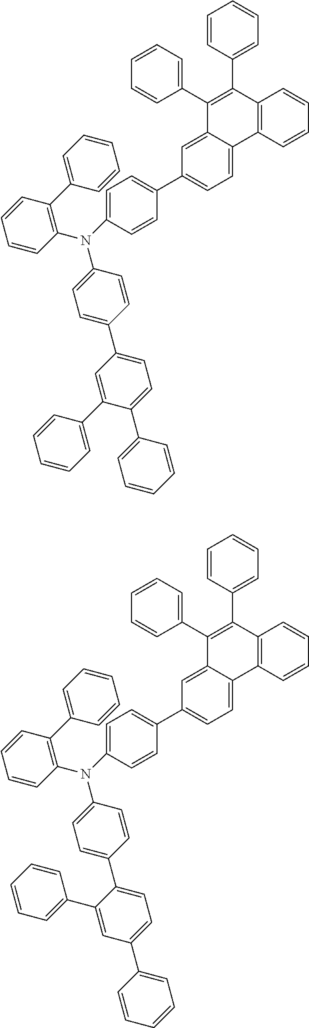 [SMILES: c1ccc(-c2ccc(-c3ccc(N(c4ccc(-c5ccc6c(c5)c(-c5ccccc5)c(-c5ccccc5)c5ccccc56)cc4)c4ccccc4-c4ccccc4)cc3)cc2-c2ccccc2)cc1 ; c1ccc(-c2ccc(-c3ccc(N(c4ccc(-c5ccc6c(c5)c(-c5ccccc5)c(-c5ccccc5)c5ccccc56)cc4)c4ccccc4-c4ccccc4)cc3)c(-c3ccccc3)c2)cc1]

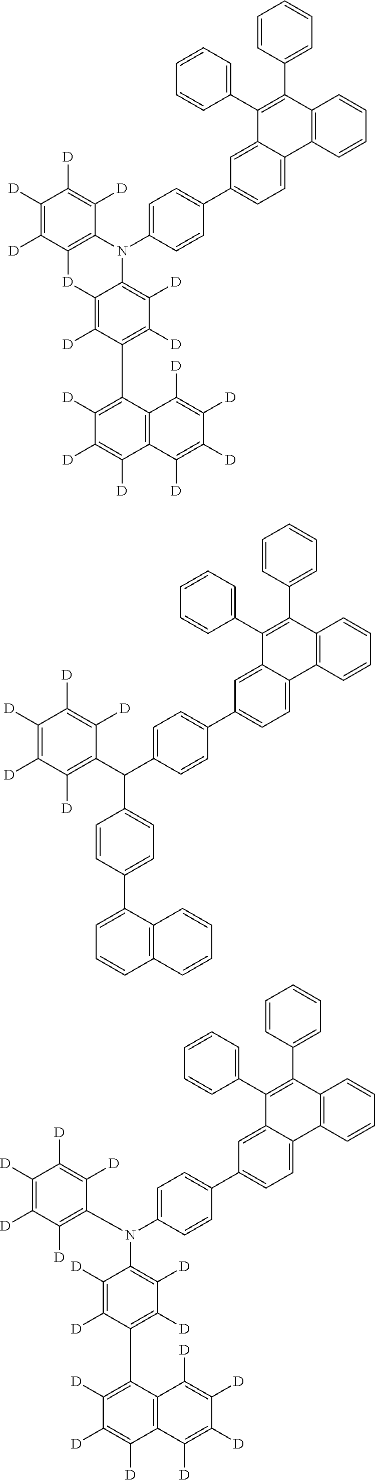

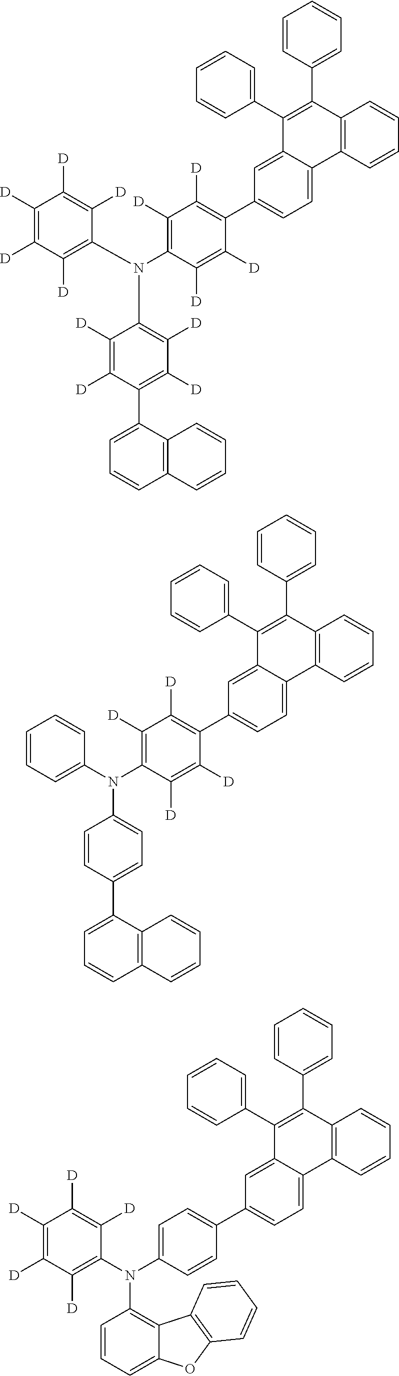 [SMILES: [2H]c1c([2H])c([2H])c(N(c2c([2H])c([2H])c(-c3ccc4c(c3)c(-c3ccccc3)c(-c3ccccc3)c3ccccc34)c([2H])c2[2H])c2c([2H])c([2H])c(-c3cccc4ccccc34)c([2H])c2[2H])c([2H])c1[2H] ; [2H]c1c([2H])c(N(c2ccccc2)c2ccc(-c3cccc4ccccc34)cc2)c([2H])c([2H])c1-c1ccc2c(c1)c(-c1ccccc1)c(-c1ccccc1)c1ccccc12 ; [2H]c1c([2H])c([2H])c(N(c2ccc(-c3ccc4c(c3)c(-c3ccccc3)c(-c3ccccc3)c3ccccc34)cc2)c2cccc3oc4ccccc4c23)c([2H])c1[2H]]

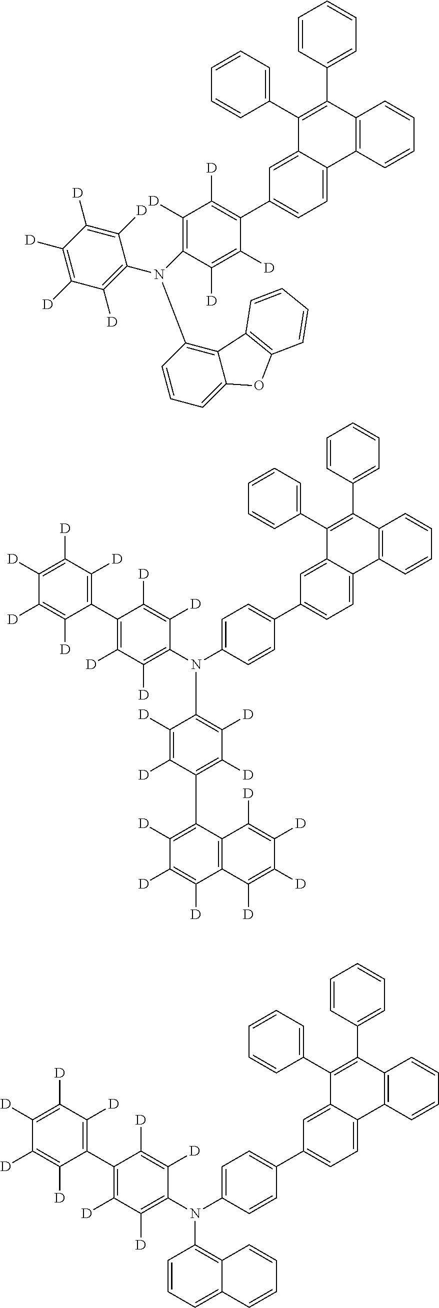 [SMILES: [2H]c1c([2H])c([2H])c(N(c2c([2H])c([2H])c(-c3ccc4c(c3)c(-c3ccccc3)c(-c3ccccc3)c3ccccc34)c([2H])c2[2H])c2cccc3oc4ccccc4c23)c([2H])c1[2H] ; [2H]c1c([2H])c([2H])c(-c2c([2H])c([2H])c(N(c3ccc(-c4ccc5c(c4)c(-c4ccccc4)c(-c4ccccc4)c4ccccc45)cc3)c3c([2H])c([2H])c(-c4c([2H])c([2H])c([2H])c5c([2H])c([2H])c([2H])c([2H])c45)c([2H])c3[2H])c([2H])c2[2H])c([2H])c1[2H] ; [2H]c1c([2H])c([2H])c(-c2c([2H])c([2H])c(N(c3ccc(-c4ccc5c(c4)c(-c4ccccc4)c(-c4ccccc4)c4ccccc45)cc3)c3cccc4ccccc34)c([2H])c2[2H])c([2H])c1[2H]]

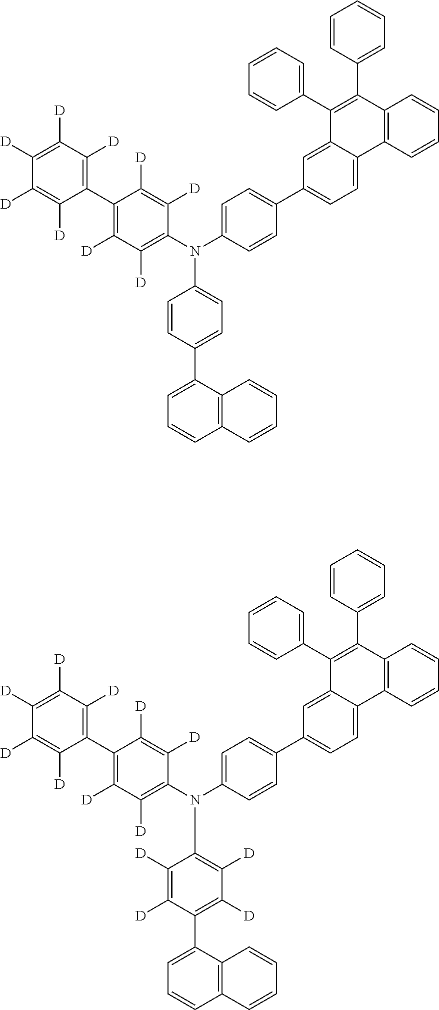 [SMILES: [2H]c1c([2H])c([2H])c(-c2c([2H])c([2H])c(N(c3ccc(-c4ccc5c(c4)c(-c4ccccc4)c(-c4ccccc4)c4ccccc45)cc3)c3ccc(-c4cccc5ccccc45)cc3)c([2H])c2[2H])c([2H])c1[2H] ; [2H]c1c([2H])c([2H])c(-c2c([2H])c([2H])c(N(c3ccc(-c4ccc5c(c4)c(-c4ccccc4)c(-c4ccccc4)c4ccccc45)cc3)c3c([2H])c([2H])c(-c4cccc5ccccc45)c([2H])c3[2H])c([2H])c2[2H])c([2H])c1[2H]]

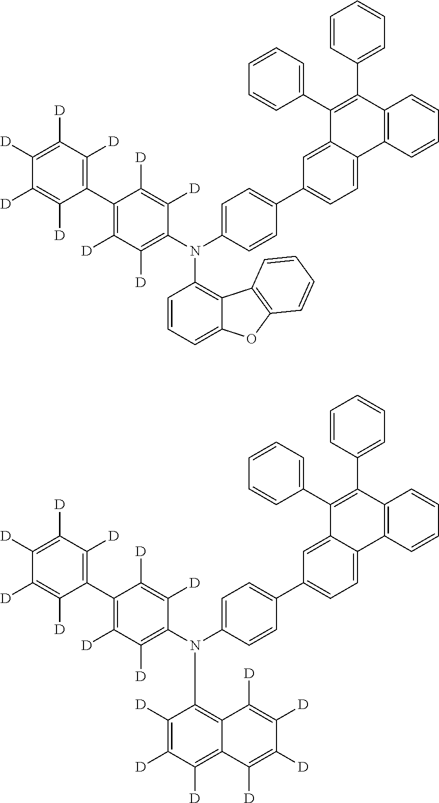

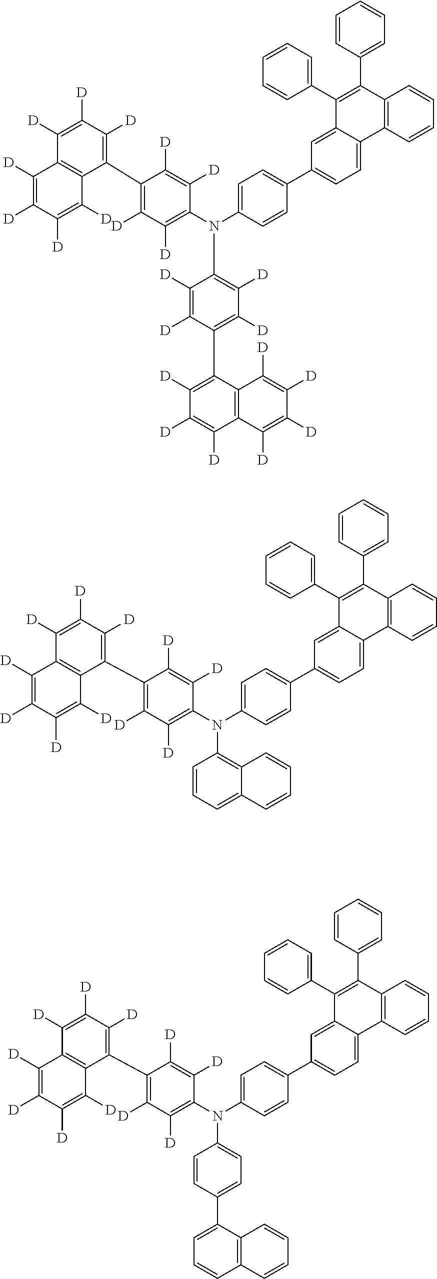

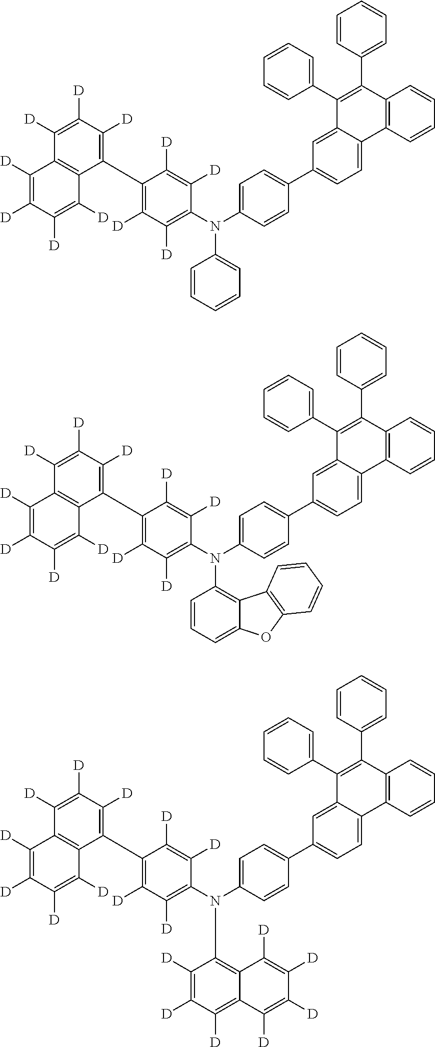

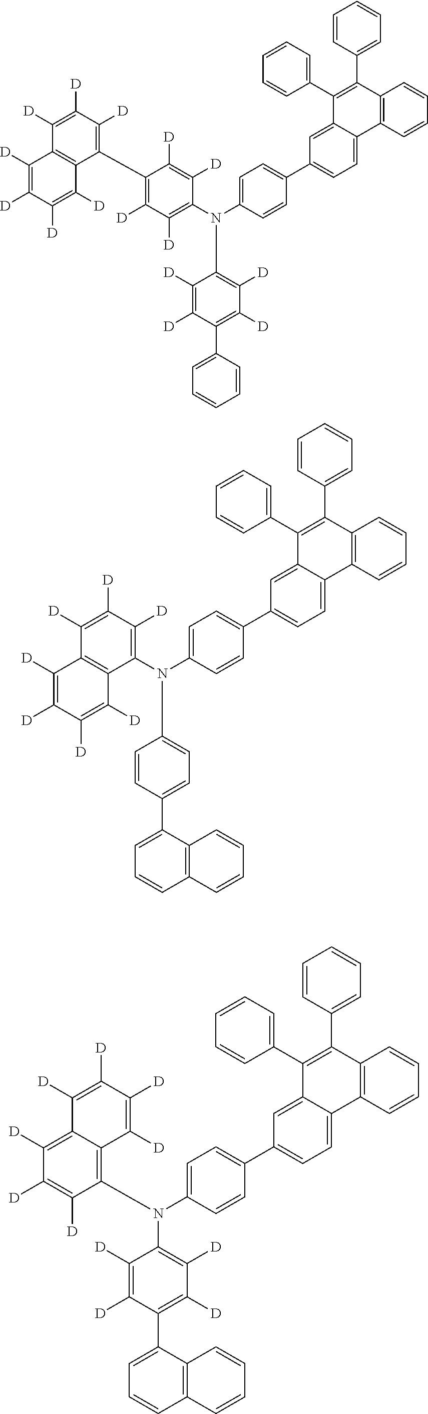 [SMILES: [2H]c1c([2H])c(N(c2ccc(-c3ccc4c(c3)c(-c3ccccc3)c(-c3ccccc3)c3ccccc34)cc2)c2c([2H])c([2H])c(-c3c([2H])c([2H])c([2H])c4c([2H])c([2H])c([2H])c([2H])c34)c([2H])c2[2H])c([2H])c([2H])c1-c1ccccc1 ; [2H]c1c([2H])c([2H])c2c(N(c3ccc(-c4ccc5c(c4)c(-c4ccccc4)c(-c4ccccc4)c4ccccc45)cc3)c3ccc(-c4cccc5ccccc45)cc3)c([2H])c([2H])c([2H])c2c1[2H] ; [2H]c1c([2H])c(N(c2ccc(-c3ccc4c(c3)c(-c3ccccc3)c(-c3ccccc3)c3ccccc34)cc2)c2c([2H])c([2H])c([2H])c3c([2H])c([2H])c([2H])c([2H])c23)c([2H])c([2H])c1-c1cccc2ccccc12]

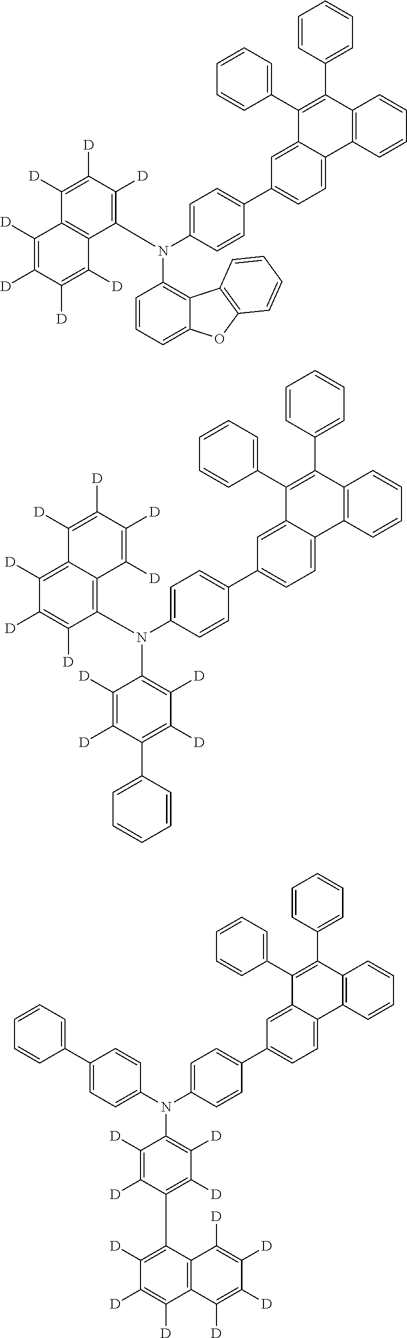 [SMILES: [2H]c1c([2H])c([2H])c2c(N(c3ccc(-c4ccc5c(c4)c(-c4ccccc4)c(-c4ccccc4)c4ccccc45)cc3)c3cccc4oc5ccccc5c34)c([2H])c([2H])c([2H])c2c1[2H] ; [2H]c1c([2H])c(N(c2ccc(-c3ccc4c(c3)c(-c3ccccc3)c(-c3ccccc3)c3ccccc34)cc2)c2c([2H])c([2H])c([2H])c3c([2H])c([2H])c([2H])c([2H])c23)c([2H])c([2H])c1-c1ccccc1 ; [2H]c1c([2H])c(N(c2ccc(-c3ccccc3)cc2)c2ccc(-c3ccc4c(c3)c(-c3ccccc3)c(-c3ccccc3)c3ccccc34)cc2)c([2H])c([2H])c1-c1c([2H])c([2H])c([2H])c2c([2H])c([2H])c([2H])c([2H])c12]

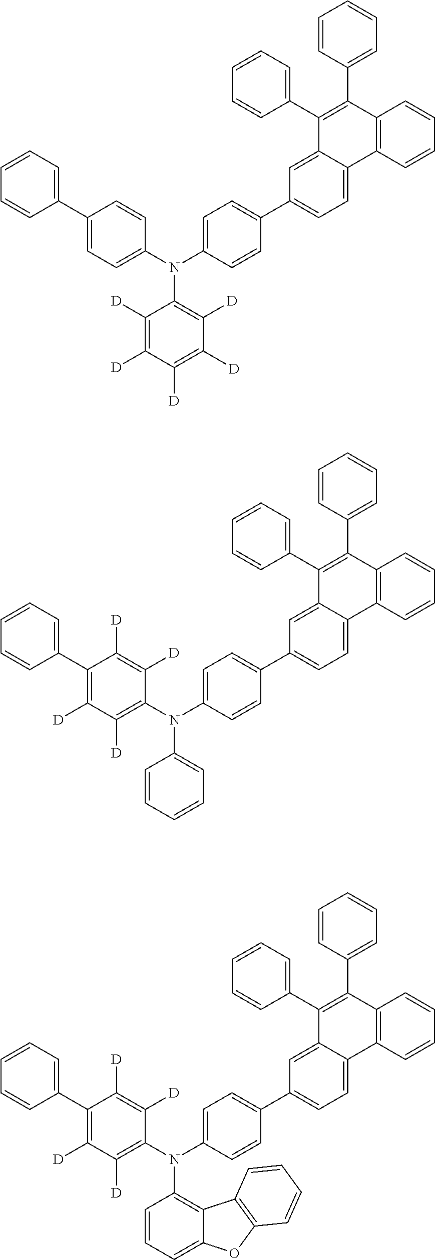 [SMILES: [2H]c1c([2H])c([2H])c(N(c2ccc(-c3ccccc3)cc2)c2ccc(-c3ccc4c(c3)c(-c3ccccc3)c(-c3ccccc3)c3ccccc34)cc2)c([2H])c1[2H] ; [2H]c1c([2H])c(N(c2ccccc2)c2ccc(-c3ccc4c(c3)c(-c3ccccc3)c(-c3ccccc3)c3ccccc34)cc2)c([2H])c([2H])c1-c1ccccc1 ; [2H]c1c([2H])c(N(c2ccc(-c3ccc4c(c3)c(-c3ccccc3)c(-c3ccccc3)c3ccccc34)cc2)c2cccc3oc4ccccc4c23)c([2H])c([2H])c1-c1ccccc1]

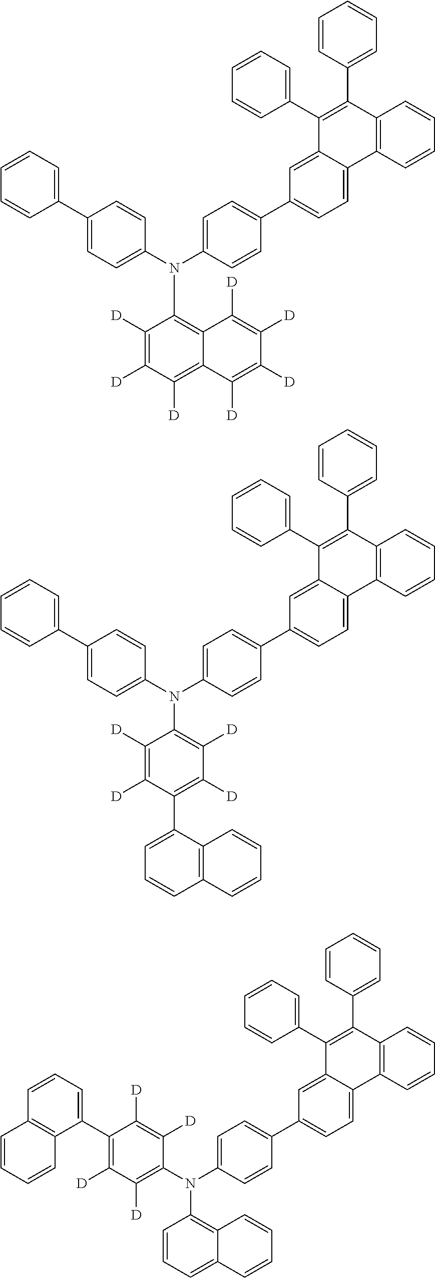 [SMILES: [2H]c1c([2H])c([2H])c2c(N(c3ccc(-c4ccccc4)cc3)c3ccc(-c4ccc5c(c4)c(-c4ccccc4)c(-c4ccccc4)c4ccccc45)cc3)c([2H])c([2H])c([2H])c2c1[2H] ; [2H]c1c([2H])c(N(c2ccc(-c3ccccc3)cc2)c2ccc(-c3ccc4c(c3)c(-c3ccccc3)c(-c3ccccc3)c3ccccc34)cc2)c([2H])c([2H])c1-c1cccc2ccccc12 ; [2H]c1c([2H])c(N(c2ccc(-c3ccc4c(c3)c(-c3ccccc3)c(-c3ccccc3)c3ccccc34)cc2)c2cccc3ccccc23)c([2H])c([2H])c1-c1cccc2ccccc12]

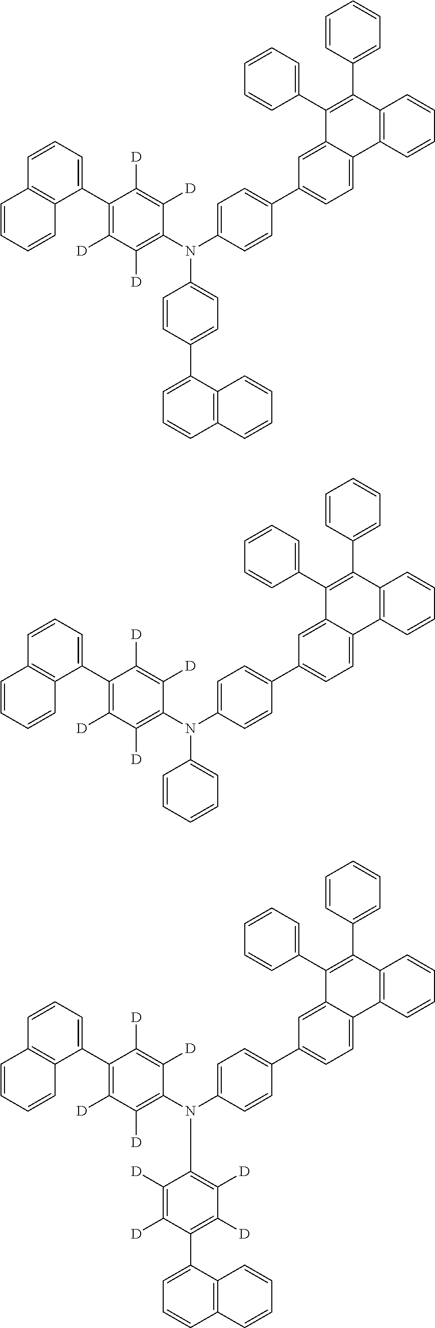 [SMILES: [2H]c1c([2H])c(N(c2ccc(-c3ccc4c(c3)c(-c3ccccc3)c(-c3ccccc3)c3ccccc34)cc2)c2ccc(-c3cccc4ccccc34)cc2)c([2H])c([2H])c1-c1cccc2ccccc12 ; [2H]c1c([2H])c(N(c2ccccc2)c2ccc(-c3ccc4c(c3)c(-c3ccccc3)c(-c3ccccc3)c3ccccc34)cc2)c([2H])c([2H])c1-c1cccc2ccccc12 ; [2H]c1c([2H])c(N(c2ccc(-c3ccc4c(c3)c(-c3ccccc3)c(-c3ccccc3)c3ccccc34)cc2)c2c([2H])c([2H])c(-c3cccc4ccccc34)c([2H])c2[2H])c([2H])c([2H])c1-c1cccc2ccccc12]

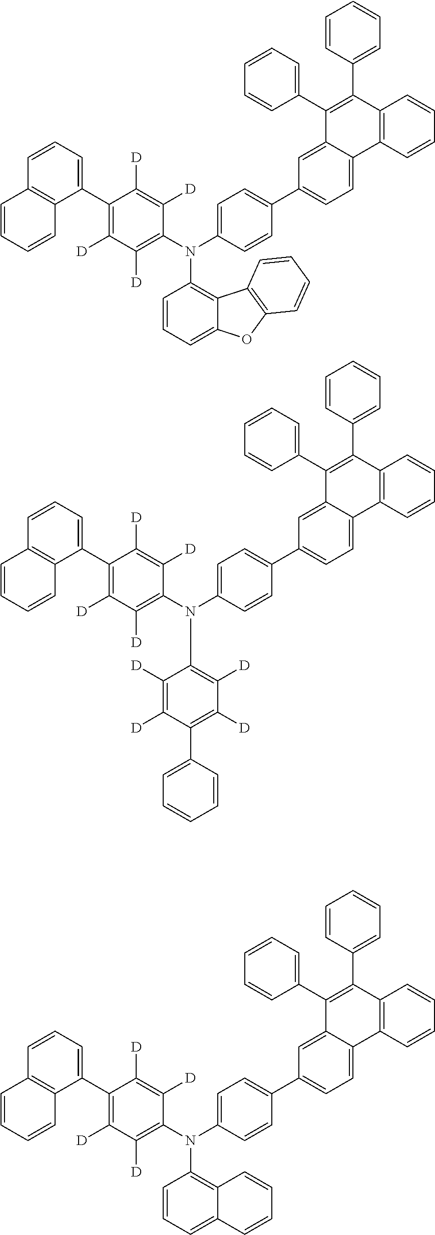 [SMILES: [2H]c1c([2H])c(N(c2ccc(-c3ccc4c(c3)c(-c3ccccc3)c(-c3ccccc3)c3ccccc34)cc2)c2cccc3oc4ccccc4c23)c([2H])c([2H])c1-c1cccc2ccccc12 ; [2H]c1c([2H])c(N(c2ccc(-c3ccc4c(c3)c(-c3ccccc3)c(-c3ccccc3)c3ccccc34)cc2)c2c([2H])c([2H])c(-c3cccc4ccccc34)c([2H])c2[2H])c([2H])c([2H])c1-c1ccccc1 ; [2H]c1c([2H])c(N(c2ccc(-c3ccc4c(c3)c(-c3ccccc3)c(-c3ccccc3)c3ccccc34)cc2)c2cccc3ccccc23)c([2H])c([2H])c1-c1cccc2ccccc12]

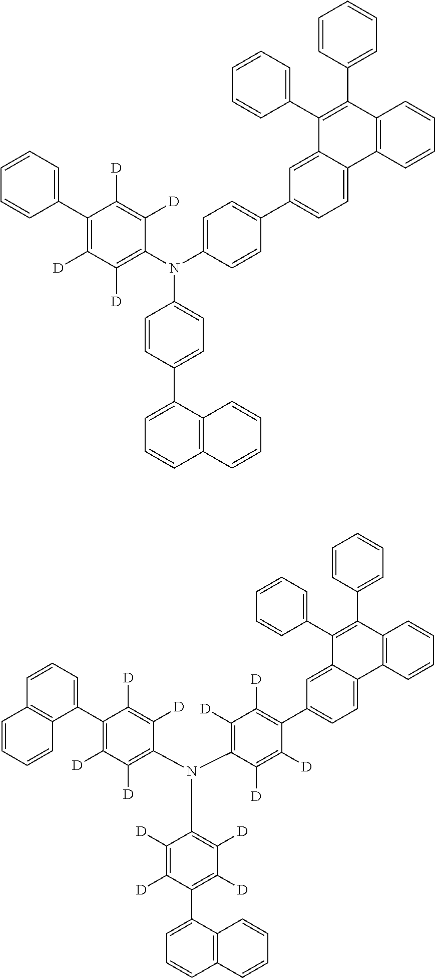 [SMILES: [2H]c1c([2H])c(N(c2ccc(-c3ccc4c(c3)c(-c3ccccc3)c(-c3ccccc3)c3ccccc34)cc2)c2ccc(-c3cccc4ccccc34)cc2)c([2H])c([2H])c1-c1ccccc1 ; [2H]c1c([2H])c(N(c2c([2H])c([2H])c(-c3cccc4ccccc34)c([2H])c2[2H])c2c([2H])c([2H])c(-c3cccc4ccccc34)c([2H])c2[2H])c([2H])c([2H])c1-c1ccc2c(c1)c(-c1ccccc1)c(-c1ccccc1)c1ccccc12]

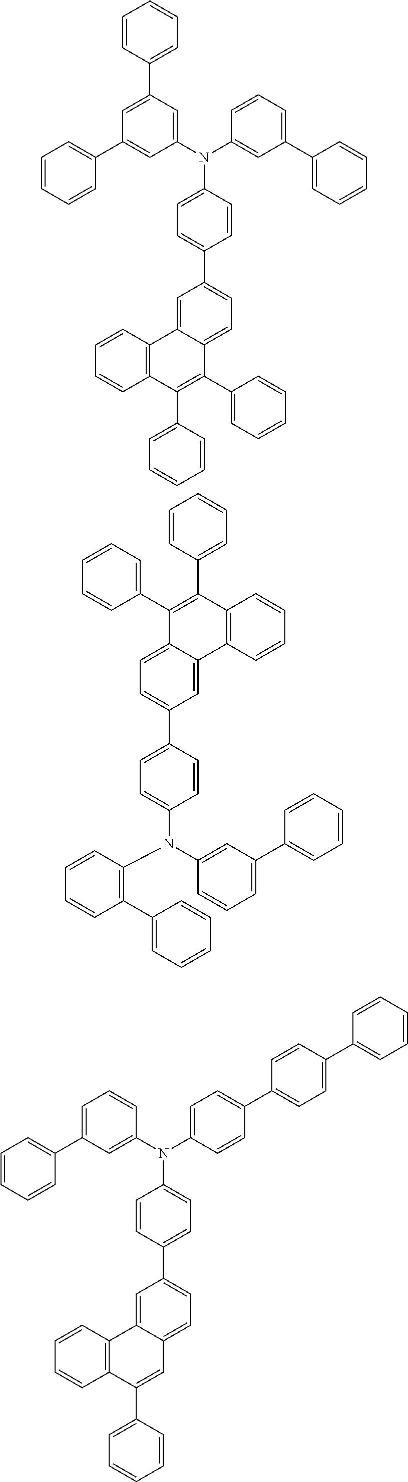

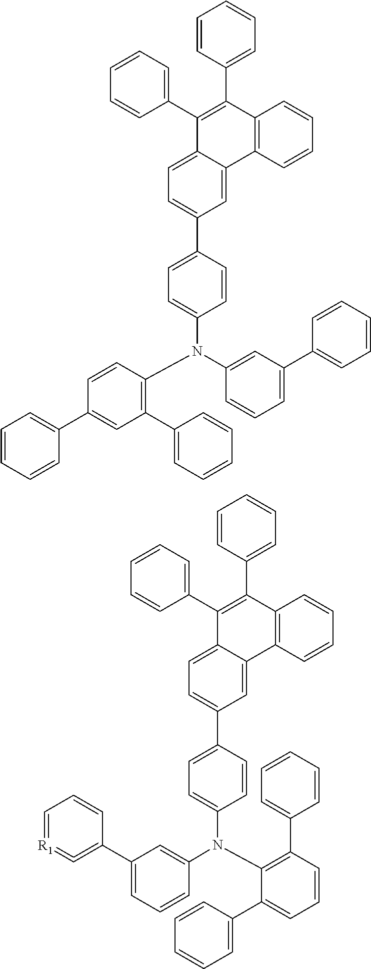

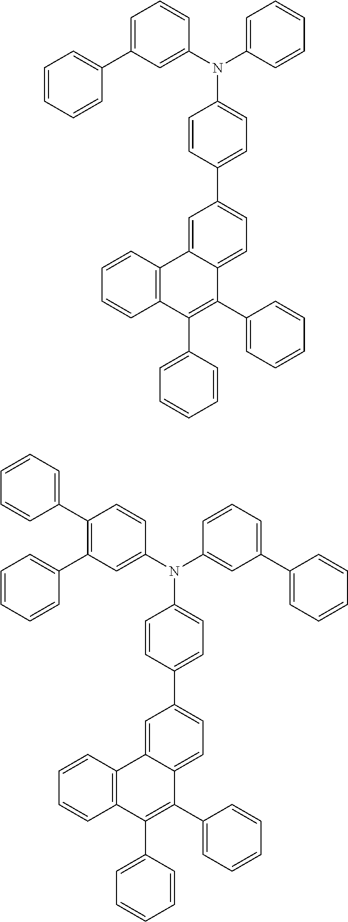 [SMILES: c1ccc(-c2cccc(N(c3ccccc3)c3ccc(-c4ccc5c(-c6ccccc6)c(-c6ccccc6)c6ccccc6c5c4)cc3)c2)cc1 ; c1ccc(-c2cccc(N(c3ccc(-c4ccc5c(-c6ccccc6)c(-c6ccccc6)c6ccccc6c5c4)cc3)c3ccc(-c4ccccc4)c(-c4ccccc4)c3)c2)cc1]

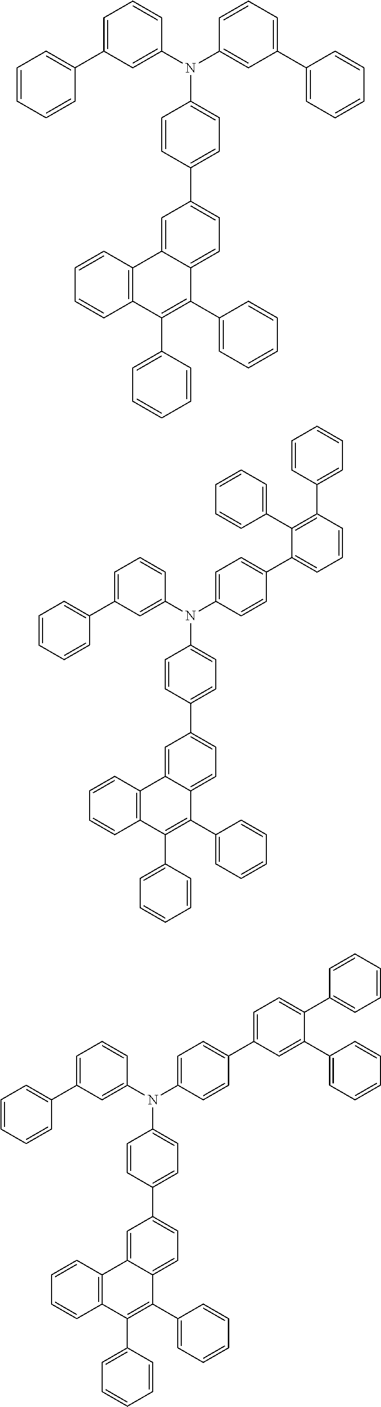 [SMILES: c1ccc(-c2cccc(N(c3ccc(-c4ccc5c(-c6ccccc6)c(-c6ccccc6)c6ccccc6c5c4)cc3)c3cccc(-c4ccccc4)c3)c2)cc1 ; c1ccc(-c2cccc(N(c3ccc(-c4ccc5c(-c6ccccc6)c(-c6ccccc6)c6ccccc6c5c4)cc3)c3ccc(-c4cccc(-c5ccccc5)c4-c4ccccc4)cc3)c2)cc1 ; c1ccc(-c2cccc(N(c3ccc(-c4ccc(-c5ccccc5)c(-c5ccccc5)c4)cc3)c3ccc(-c4ccc5c(-c6ccccc6)c(-c6ccccc6)c6ccccc6c5c4)cc3)c2)cc1]

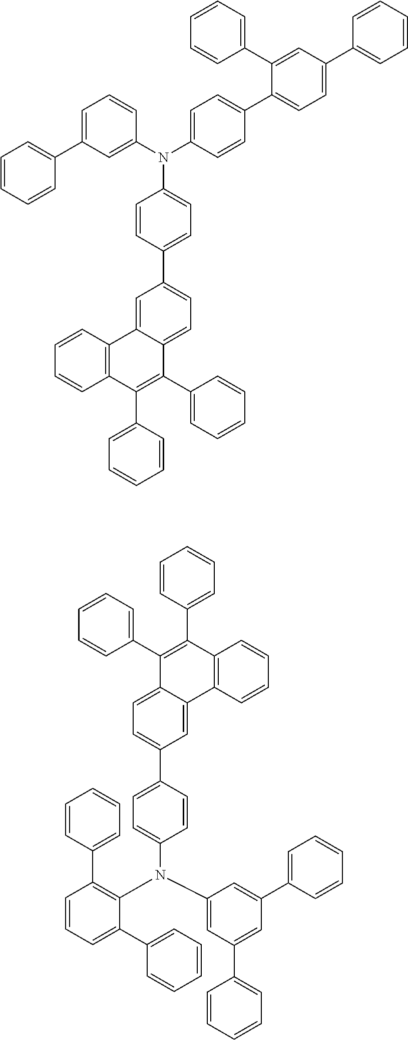 [SMILES: c1ccc(-c2cccc(N(c3ccc(-c4ccc5c(-c6ccccc6)c(-c6ccccc6)c6ccccc6c5c4)cc3)c3ccc(-c4ccc(-c5ccccc5)cc4-c4ccccc4)cc3)c2)cc1 ; c1ccc(-c2cc(-c3ccccc3)cc(N(c3ccc(-c4ccc5c(-c6ccccc6)c(-c6ccccc6)c6ccccc6c5c4)cc3)c3c(-c4ccccc4)cccc3-c3ccccc3)c2)cc1]

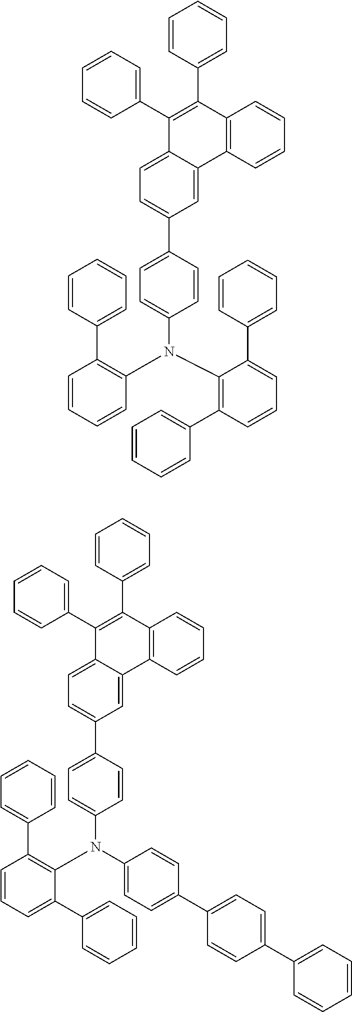 [SMILES: c1ccc(-c2ccccc2N(c2ccc(-c3ccc4c(-c5ccccc5)c(-c5ccccc5)c5ccccc5c4c3)cc2)c2c(-c3ccccc3)cccc2-c2ccccc2)cc1 ; c1ccc(-c2ccc(-c3ccc(N(c4ccc(-c5ccc6c(-c7ccccc7)c(-c7ccccc7)c7ccccc7c6c5)cc4)c4c(-c5ccccc5)cccc4-c4ccccc4)cc3)cc2)cc1]

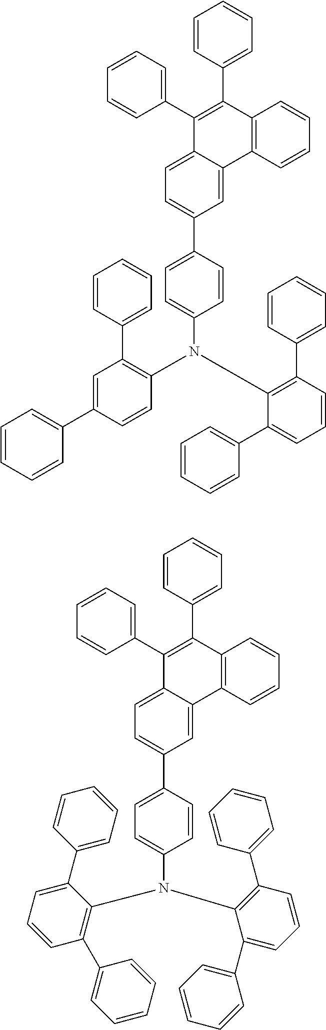

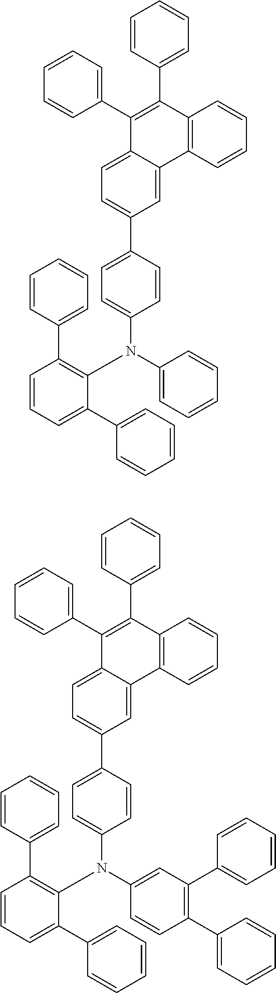

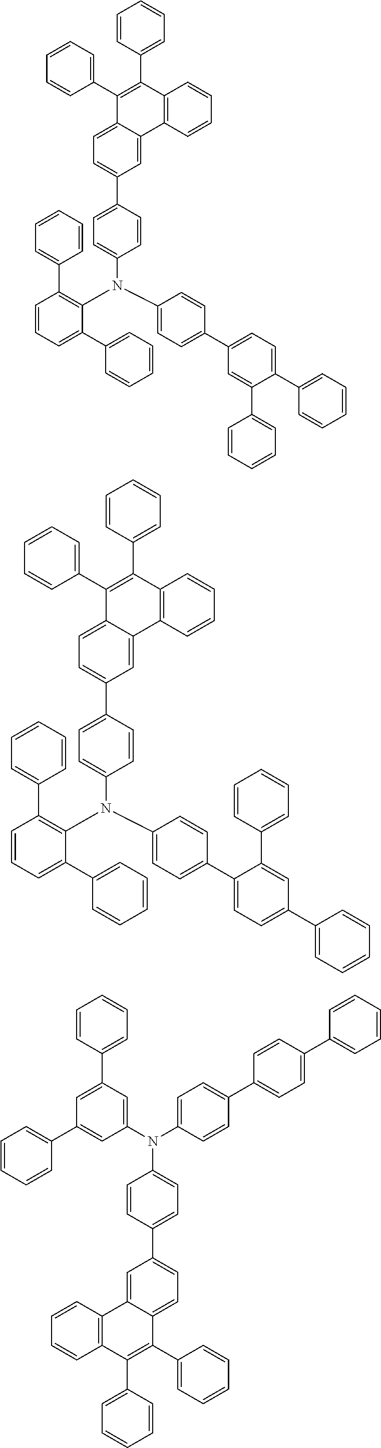

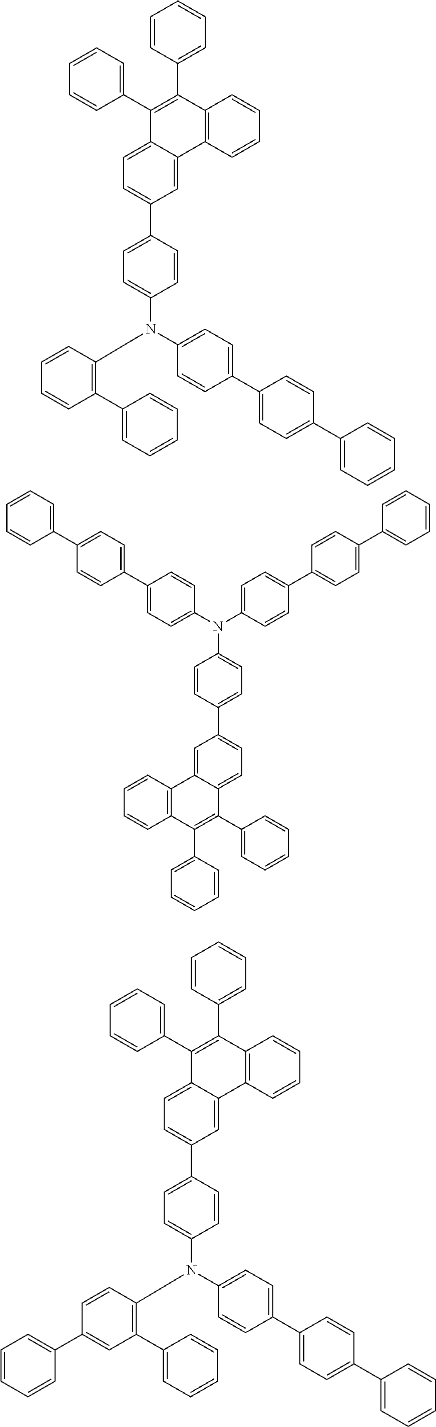

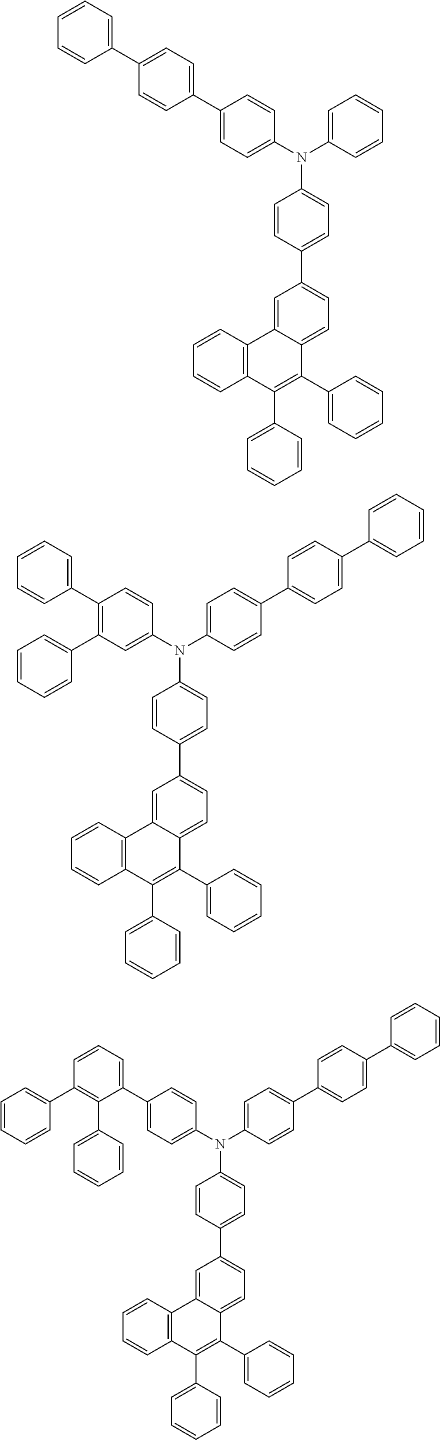 [SMILES: c1ccc(-c2ccc(-c3ccc(N(c4ccccc4)c4ccc(-c5ccc6c(-c7ccccc7)c(-c7ccccc7)c7ccccc7c6c5)cc4)cc3)cc2)cc1 ; c1ccc(-c2ccc(-c3ccc(N(c4ccc(-c5ccc6c(-c7ccccc7)c(-c7ccccc7)c7ccccc7c6c5)cc4)c4ccc(-c5ccccc5)c(-c5ccccc5)c4)cc3)cc2)cc1 ; c1ccc(-c2ccc(-c3ccc(N(c4ccc(-c5ccc6c(-c7ccccc7)c(-c7ccccc7)c7ccccc7c6c5)cc4)c4ccc(-c5cccc(-c6ccccc6)c5-c5ccccc5)cc4)cc3)cc2)cc1]

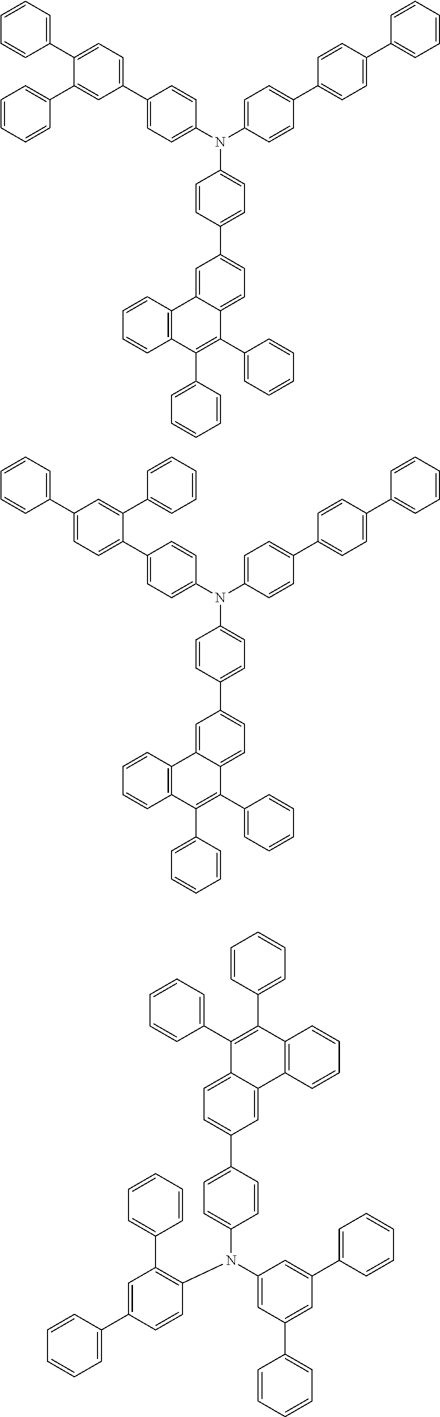

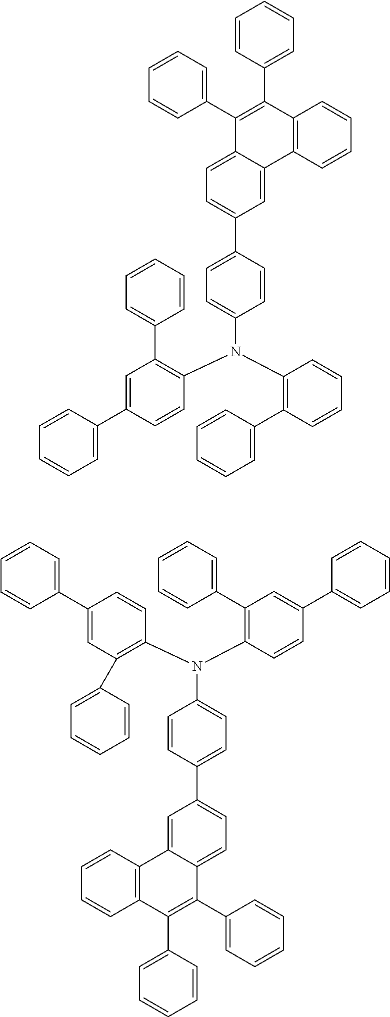 [SMILES: c1ccc(-c2ccc(N(c3ccc(-c4ccc5c(-c6ccccc6)c(-c6ccccc6)c6ccccc6c5c4)cc3)c3ccccc3-c3ccccc3)c(-c3ccccc3)c2)cc1 ; c1ccc(-c2ccc(N(c3ccc(-c4ccc5c(-c6ccccc6)c(-c6ccccc6)c6ccccc6c5c4)cc3)c3ccc(-c4ccccc4)cc3-c3ccccc3)c(-c3ccccc3)c2)cc1]

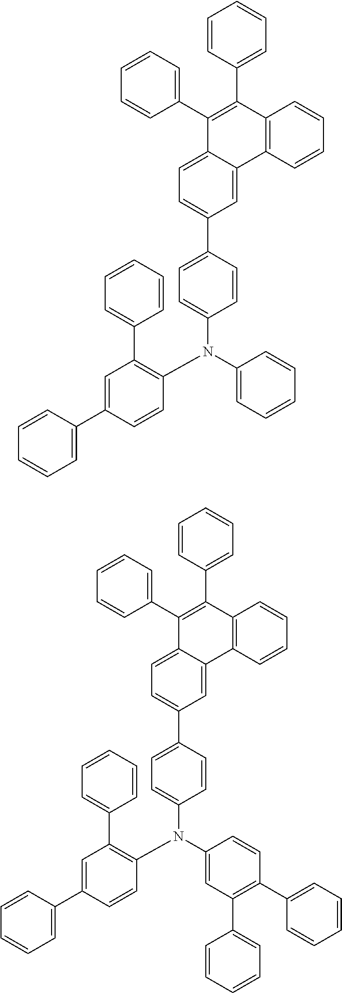 [SMILES: c1ccc(-c2ccc(N(c3ccccc3)c3ccc(-c4ccc5c(-c6ccccc6)c(-c6ccccc6)c6ccccc6c5c4)cc3)c(-c3ccccc3)c2)cc1 ; c1ccc(-c2ccc(N(c3ccc(-c4ccc5c(-c6ccccc6)c(-c6ccccc6)c6ccccc6c5c4)cc3)c3ccc(-c4ccccc4)c(-c4ccccc4)c3)c(-c3ccccc3)c2)cc1]

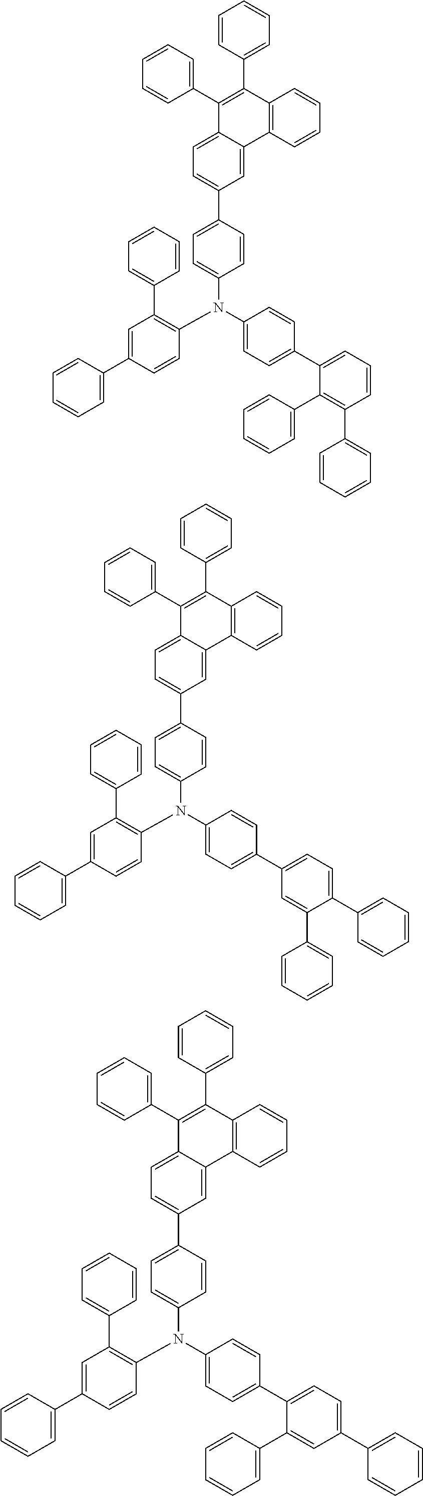 [SMILES: c1ccc(-c2ccc(N(c3ccc(-c4ccc5c(-c6ccccc6)c(-c6ccccc6)c6ccccc6c5c4)cc3)c3ccc(-c4cccc(-c5ccccc5)c4-c4ccccc4)cc3)c(-c3ccccc3)c2)cc1 ; c1ccc(-c2ccc(N(c3ccc(-c4ccc(-c5ccccc5)c(-c5ccccc5)c4)cc3)c3ccc(-c4ccc5c(-c6ccccc6)c(-c6ccccc6)c6ccccc6c5c4)cc3)c(-c3ccccc3)c2)cc1 ; c1ccc(-c2ccc(-c3ccc(N(c4ccc(-c5ccc6c(-c7ccccc7)c(-c7ccccc7)c7ccccc7c6c5)cc4)c4ccc(-c5ccccc5)cc4-c4ccccc4)cc3)c(-c3ccccc3)c2)cc1]

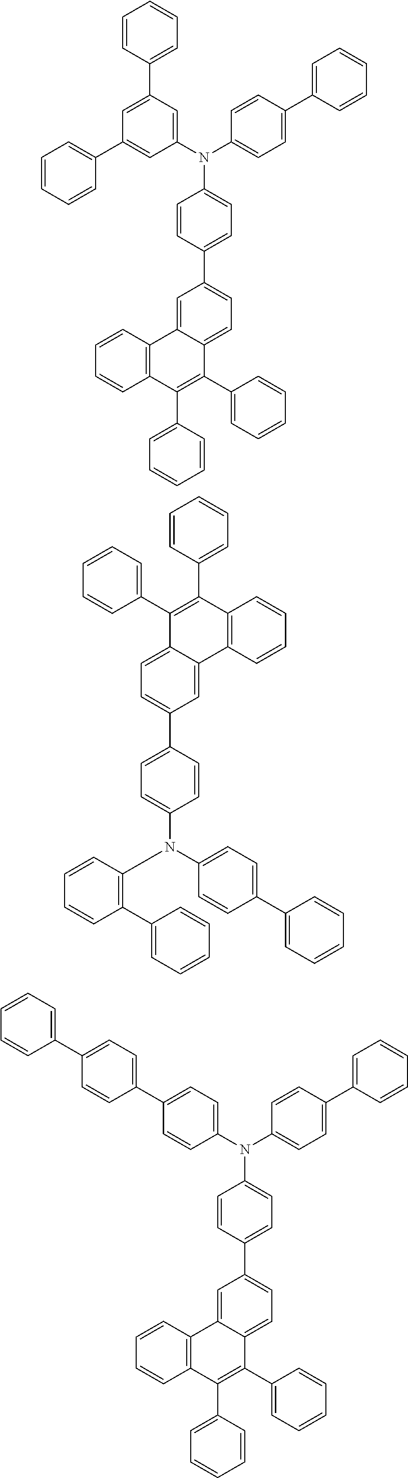

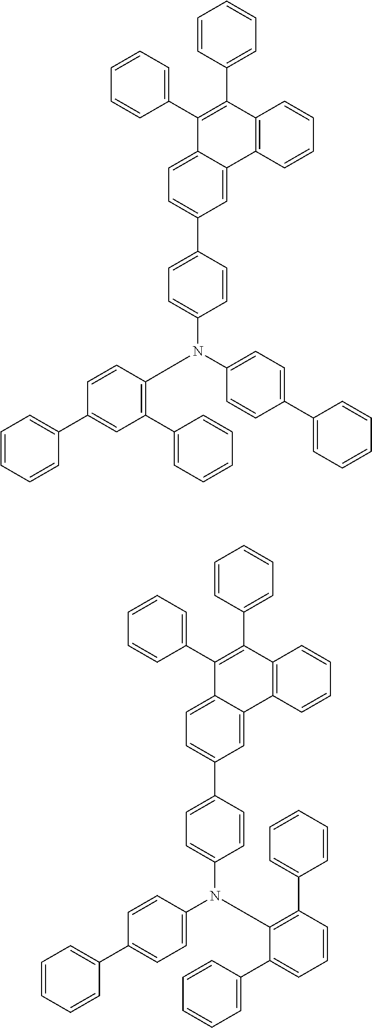 [SMILES: c1ccc(-c2ccc(N(c3ccc(-c4ccc5c(-c6ccccc6)c(-c6ccccc6)c6ccccc6c5c4)cc3)c3ccc(-c4ccccc4)cc3-c3ccccc3)cc2)cc1 ; c1ccc(-c2ccc(N(c3ccc(-c4ccc5c(-c6ccccc6)c(-c6ccccc6)c6ccccc6c5c4)cc3)c3c(-c4ccccc4)cccc3-c3ccccc3)cc2)cc1]

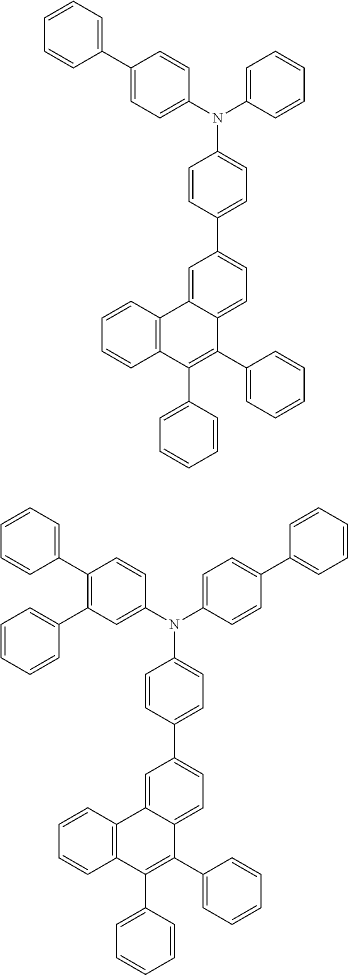 [SMILES: c1ccc(-c2ccc(N(c3ccccc3)c3ccc(-c4ccc5c(-c6ccccc6)c(-c6ccccc6)c6ccccc6c5c4)cc3)cc2)cc1 ; c1ccc(-c2ccc(N(c3ccc(-c4ccc5c(-c6ccccc6)c(-c6ccccc6)c6ccccc6c5c4)cc3)c3ccc(-c4ccccc4)c(-c4ccccc4)c3)cc2)cc1]

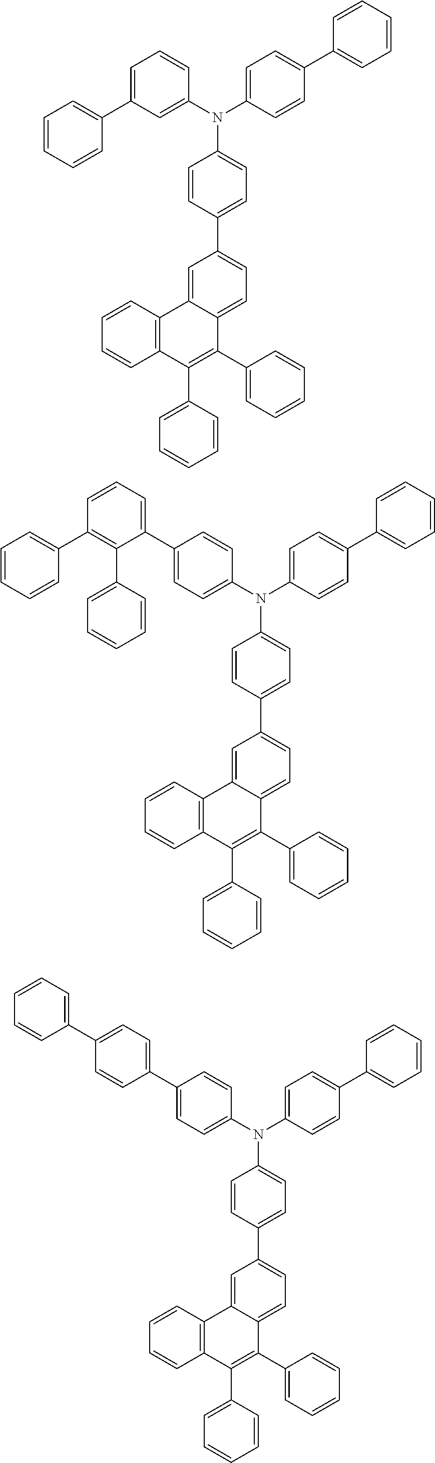 [SMILES: c1ccc(-c2ccc(N(c3ccc(-c4ccc5c(-c6ccccc6)c(-c6ccccc6)c6ccccc6c5c4)cc3)c3cccc(-c4ccccc4)c3)cc2)cc1 ; c1ccc(-c2ccc(N(c3ccc(-c4ccc5c(-c6ccccc6)c(-c6ccccc6)c6ccccc6c5c4)cc3)c3ccc(-c4cccc(-c5ccccc5)c4-c4ccccc4)cc3)cc2)cc1 ; c1ccc(-c2ccc(-c3ccc(N(c4ccc(-c5ccccc5)cc4)c4ccc(-c5ccc6c(-c7ccccc7)c(-c7ccccc7)c7ccccc7c6c5)cc4)cc3)cc2)cc1]

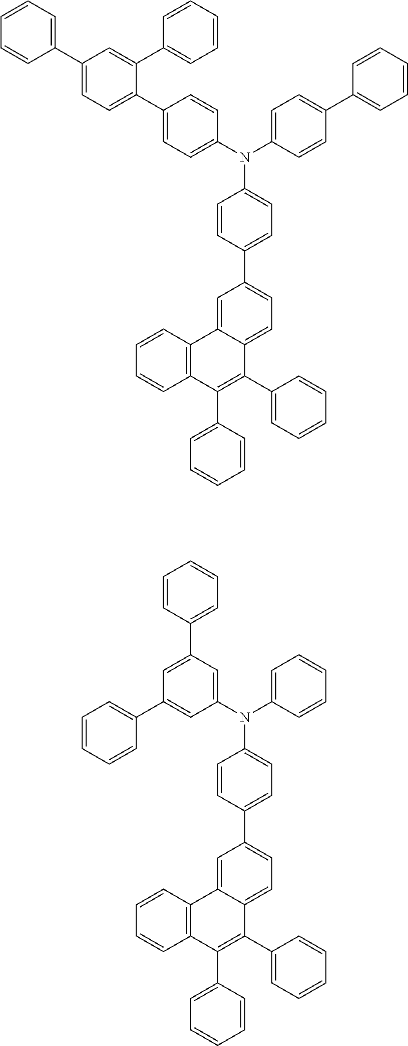 [SMILES: c1ccc(-c2ccc(N(c3ccc(-c4ccc5c(-c6ccccc6)c(-c6ccccc6)c6ccccc6c5c4)cc3)c3ccc(-c4ccc(-c5ccccc5)cc4-c4ccccc4)cc3)cc2)cc1 ; c1ccc(-c2cc(-c3ccccc3)cc(N(c3ccccc3)c3ccc(-c4ccc5c(-c6ccccc6)c(-c6ccccc6)c6ccccc6c5c4)cc3)c2)cc1]

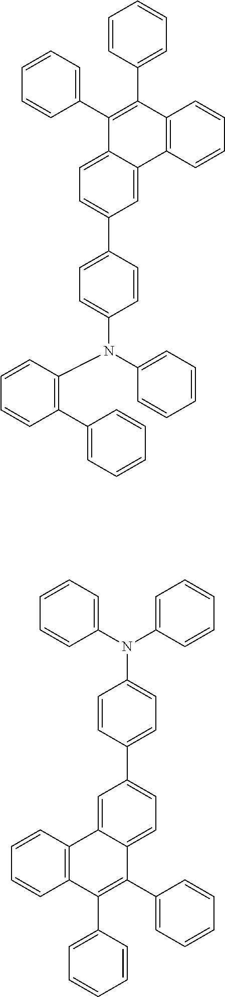 [SMILES: c1ccc(-c2ccccc2N(c2ccccc2)c2ccc(-c3ccc4c(-c5ccccc5)c(-c5ccccc5)c5ccccc5c4c3)cc2)cc1 ; c1ccc(-c2c(-c3ccccc3)c3ccc(-c4ccc(N(c5ccccc5)c5ccccc5)cc4)cc3c3ccccc23)cc1]

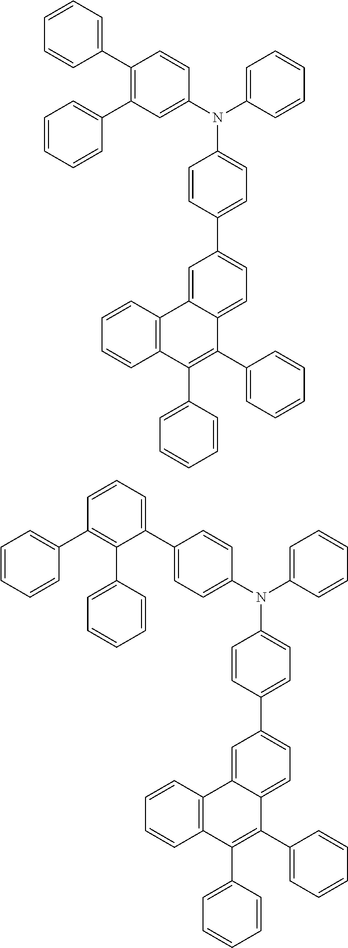 [SMILES: c1ccc(-c2ccc(N(c3ccccc3)c3ccc(-c4ccc5c(-c6ccccc6)c(-c6ccccc6)c6ccccc6c5c4)cc3)cc2-c2ccccc2)cc1 ; c1ccc(-c2cccc(-c3ccc(N(c4ccccc4)c4ccc(-c5ccc6c(-c7ccccc7)c(-c7ccccc7)c7ccccc7c6c5)cc4)cc3)c2-c2ccccc2)cc1]

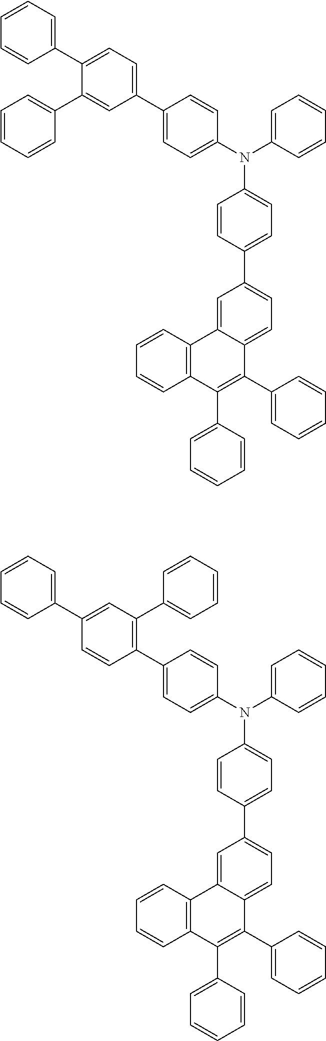 [SMILES: c1ccc(-c2ccc(-c3ccc(N(c4ccccc4)c4ccc(-c5ccc6c(-c7ccccc7)c(-c7ccccc7)c7ccccc7c6c5)cc4)cc3)cc2-c2ccccc2)cc1 ; c1ccc(-c2ccc(-c3ccc(N(c4ccccc4)c4ccc(-c5ccc6c(-c7ccccc7)c(-c7ccccc7)c7ccccc7c6c5)cc4)cc3)c(-c3ccccc3)c2)cc1]

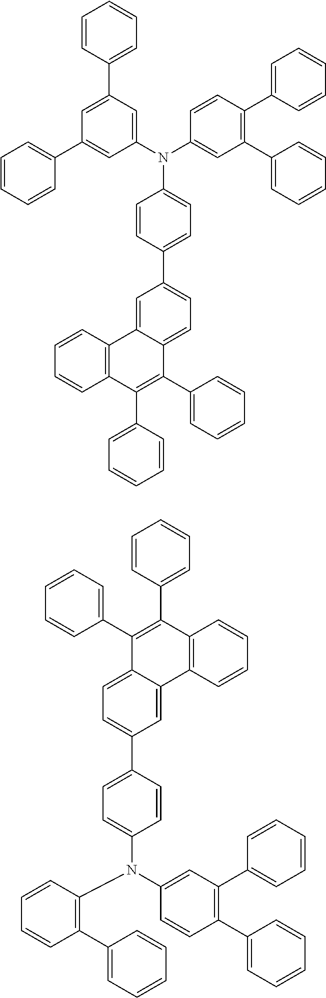

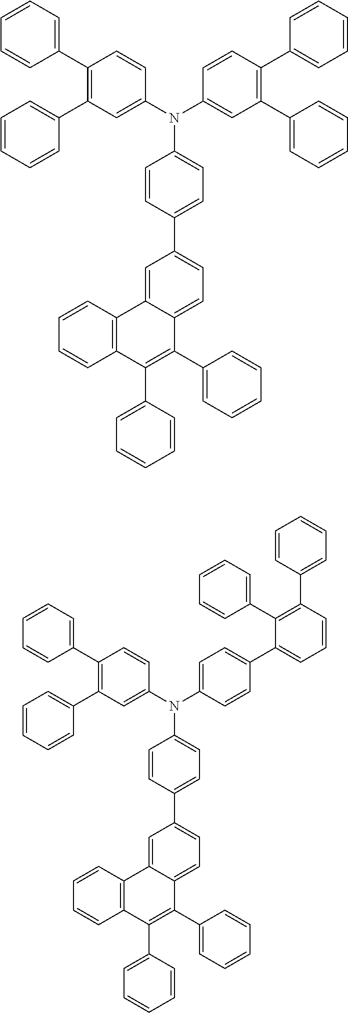 [SMILES: c1ccc(-c2ccc(N(c3ccc(-c4ccc5c(-c6ccccc6)c(-c6ccccc6)c6ccccc6c5c4)cc3)c3ccc(-c4ccccc4)c(-c4ccccc4)c3)cc2-c2ccccc2)cc1 ; c1ccc(-c2ccc(N(c3ccc(-c4ccc5c(-c6ccccc6)c(-c6ccccc6)c6ccccc6c5c4)cc3)c3ccc(-c4cccc(-c5ccccc5)c4-c4ccccc4)cc3)cc2-c2ccccc2)cc1]

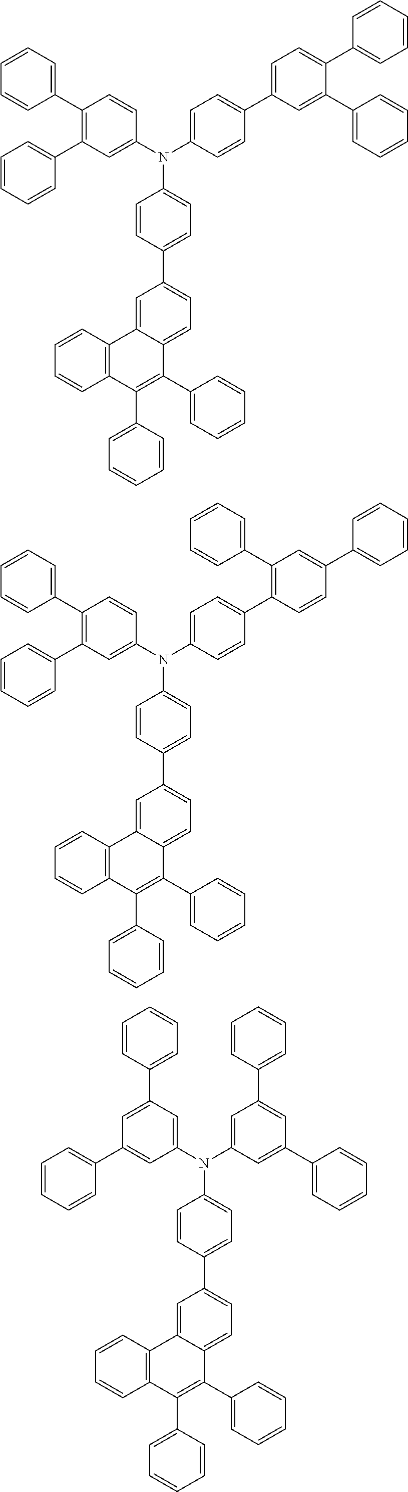

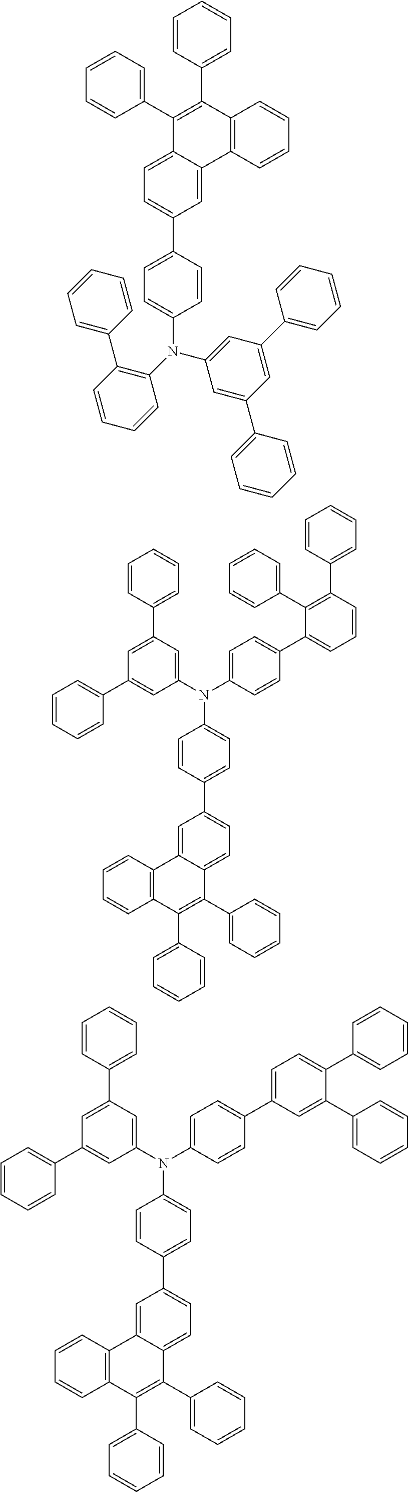

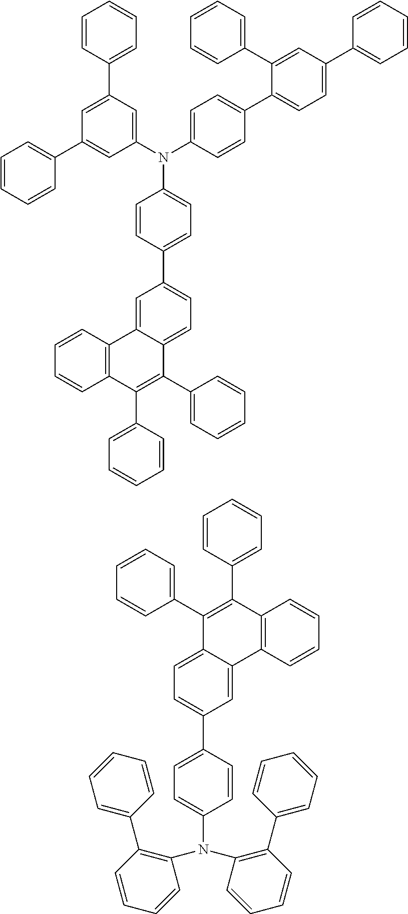 [SMILES: c1ccc(-c2cc(-c3ccccc3)cc(N(c3ccc(-c4ccc5c(-c6ccccc6)c(-c6ccccc6)c6ccccc6c5c4)cc3)c3ccc(-c4ccc(-c5ccccc5)cc4-c4ccccc4)cc3)c2)cc1 ; c1ccc(-c2ccccc2N(c2ccc(-c3ccc4c(-c5ccccc5)c(-c5ccccc5)c5ccccc5c4c3)cc2)c2ccccc2-c2ccccc2)cc1]

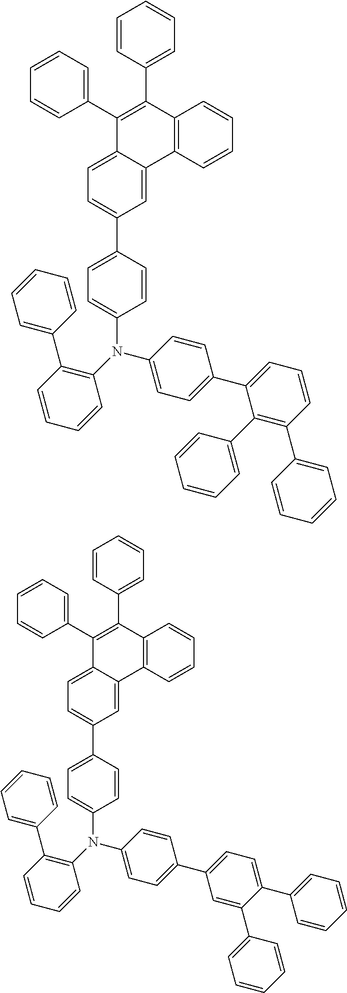 [SMILES: c1ccc(-c2ccccc2N(c2ccc(-c3ccc4c(-c5ccccc5)c(-c5ccccc5)c5ccccc5c4c3)cc2)c2ccc(-c3cccc(-c4ccccc4)c3-c3ccccc3)cc2)cc1 ; c1ccc(-c2ccc(-c3ccc(N(c4ccc(-c5ccc6c(-c7ccccc7)c(-c7ccccc7)c7ccccc7c6c5)cc4)c4ccccc4-c4ccccc4)cc3)cc2-c2ccccc2)cc1]

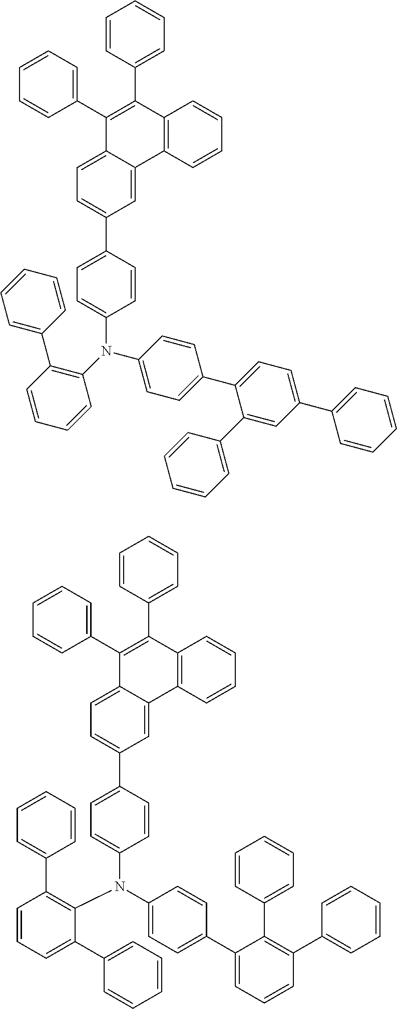 [SMILES: c1ccc(-c2ccc(-c3ccc(N(c4ccc(-c5ccc6c(-c7ccccc7)c(-c7ccccc7)c7ccccc7c6c5)cc4)c4ccccc4-c4ccccc4)cc3)c(-c3ccccc3)c2)cc1 ; c1ccc(-c2cccc(-c3ccc(N(c4ccc(-c5ccc6c(-c7ccccc7)c(-c7ccccc7)c7ccccc7c6c5)cc4)c4c(-c5ccccc5)cccc4-c4ccccc4)cc3)c2-c2ccccc2)cc1]

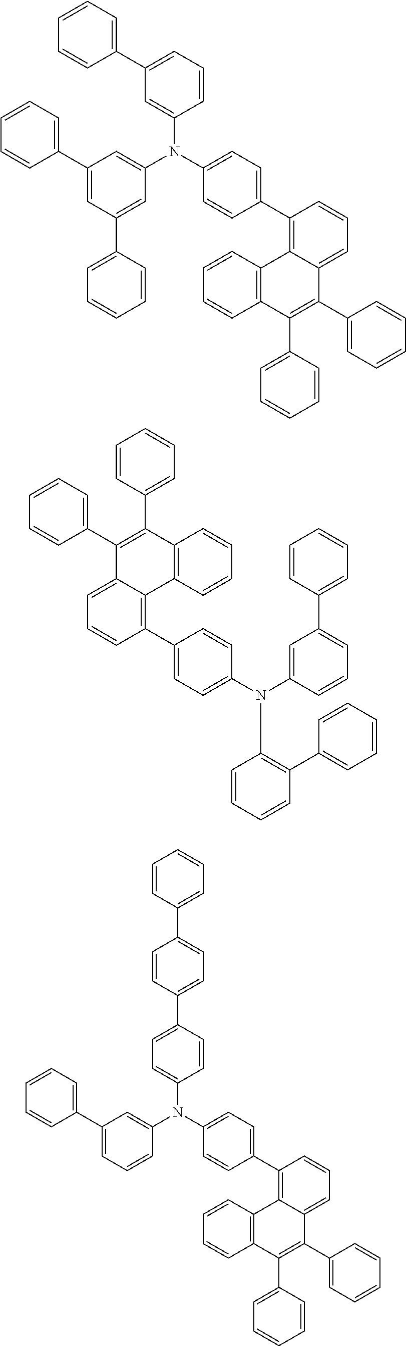

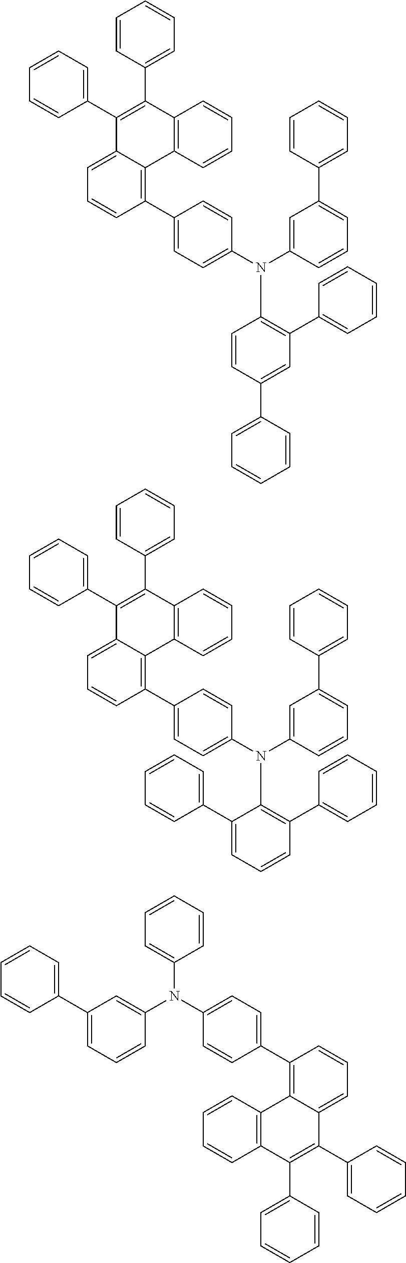 [SMILES: c1ccc(-c2cccc(N(c3ccc(-c4cccc5c(-c6ccccc6)c(-c6ccccc6)c6ccccc6c45)cc3)c3ccc(-c4ccccc4)cc3-c3ccccc3)c2)cc1 ; c1ccc(-c2cccc(N(c3ccc(-c4cccc5c(-c6ccccc6)c(-c6ccccc6)c6ccccc6c45)cc3)c3c(-c4ccccc4)cccc3-c3ccccc3)c2)cc1 ; c1ccc(-c2cccc(N(c3ccccc3)c3ccc(-c4cccc5c(-c6ccccc6)c(-c6ccccc6)c6ccccc6c45)cc3)c2)cc1]

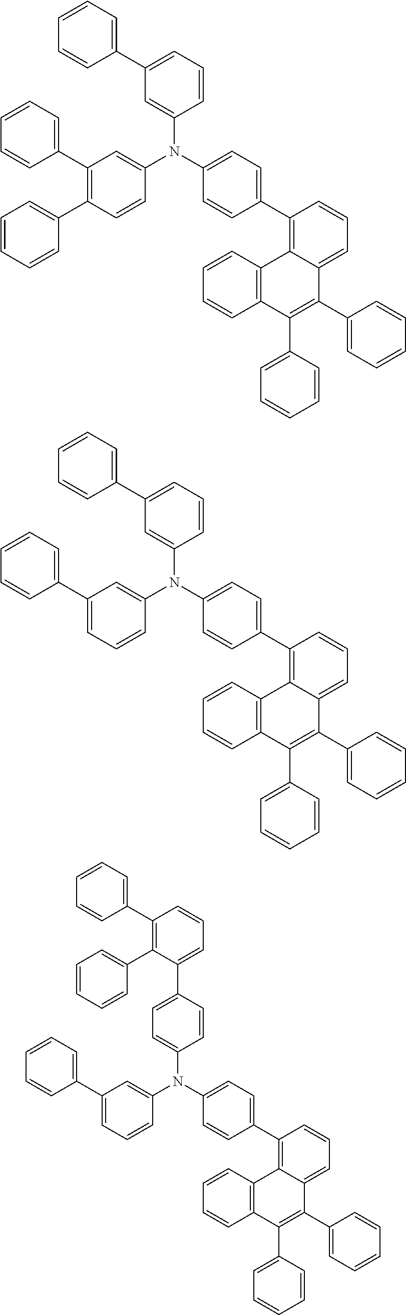 [SMILES: c1ccc(-c2cccc(N(c3ccc(-c4cccc5c(-c6ccccc6)c(-c6ccccc6)c6ccccc6c45)cc3)c3ccc(-c4ccccc4)c(-c4ccccc4)c3)c2)cc1 ; c1ccc(-c2cccc(N(c3ccc(-c4cccc5c(-c6ccccc6)c(-c6ccccc6)c6ccccc6c45)cc3)c3cccc(-c4ccccc4)c3)c2)cc1 ; c1ccc(-c2cccc(N(c3ccc(-c4cccc(-c5ccccc5)c4-c4ccccc4)cc3)c3ccc(-c4cccc5c(-c6ccccc6)c(-c6ccccc6)c6ccccc6c45)cc3)c2)cc1]

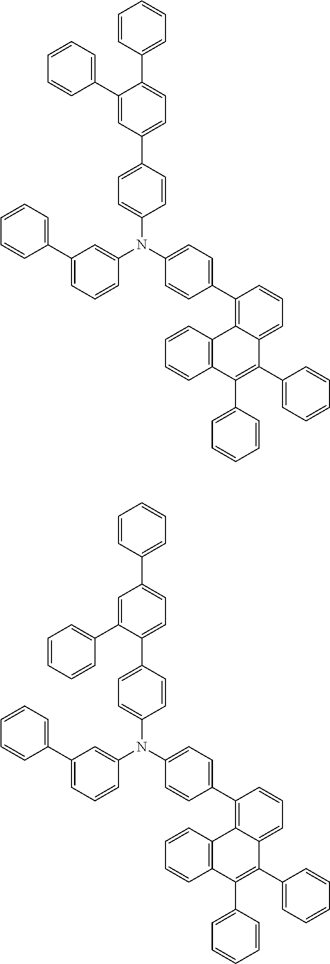 [SMILES: c1ccc(-c2cccc(N(c3ccc(-c4ccc(-c5ccccc5)c(-c5ccccc5)c4)cc3)c3ccc(-c4cccc5c(-c6ccccc6)c(-c6ccccc6)c6ccccc6c45)cc3)c2)cc1 ; c1ccc(-c2cccc(N(c3ccc(-c4ccc(-c5ccccc5)cc4-c4ccccc4)cc3)c3ccc(-c4cccc5c(-c6ccccc6)c(-c6ccccc6)c6ccccc6c45)cc3)c2)cc1]

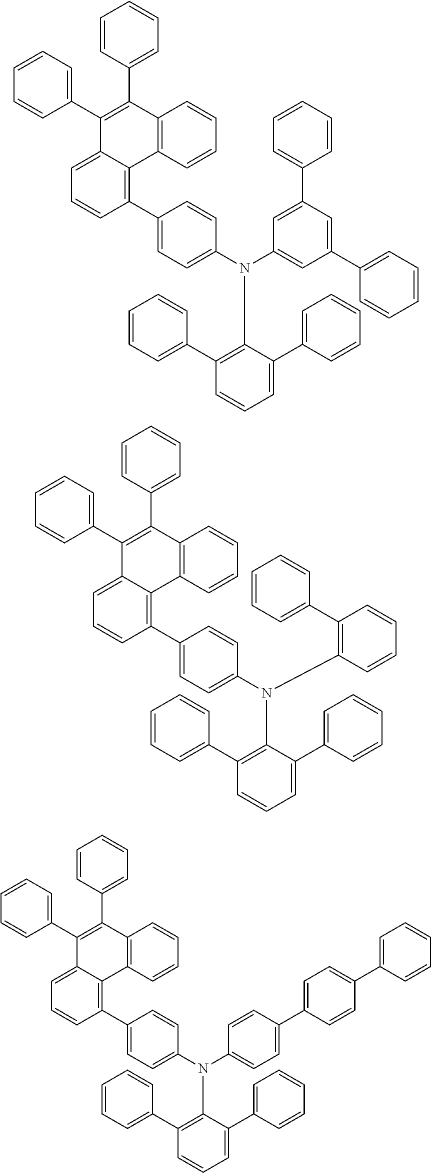 [SMILES: c1ccc(-c2cc(-c3ccccc3)cc(N(c3ccc(-c4cccc5c(-c6ccccc6)c(-c6ccccc6)c6ccccc6c45)cc3)c3c(-c4ccccc4)cccc3-c3ccccc3)c2)cc1 ; c1ccc(-c2ccccc2N(c2ccc(-c3cccc4c(-c5ccccc5)c(-c5ccccc5)c5ccccc5c34)cc2)c2c(-c3ccccc3)cccc2-c2ccccc2)cc1 ; c1ccc(-c2ccc(-c3ccc(N(c4ccc(-c5cccc6c(-c7ccccc7)c(-c7ccccc7)c7ccccc7c56)cc4)c4c(-c5ccccc5)cccc4-c4ccccc4)cc3)cc2)cc1]

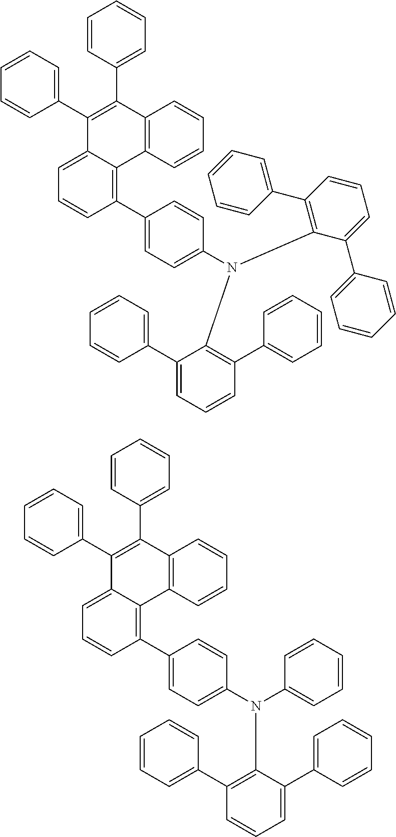 [SMILES: c1ccc(-c2cccc(-c3ccccc3)c2N(c2ccc(-c3cccc4c(-c5ccccc5)c(-c5ccccc5)c5ccccc5c34)cc2)c2c(-c3ccccc3)cccc2-c2ccccc2)cc1 ; c1ccc(-c2cccc(-c3ccccc3)c2N(c2ccccc2)c2ccc(-c3cccc4c(-c5ccccc5)c(-c5ccccc5)c5ccccc5c34)cc2)cc1]

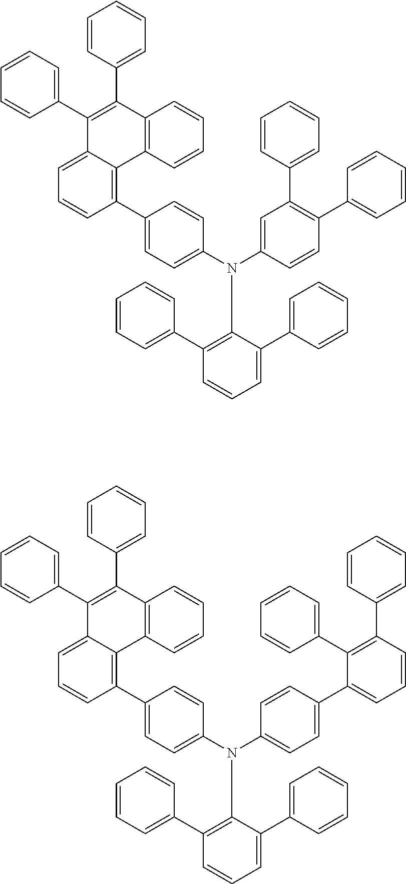 [SMILES: c1ccc(-c2ccc(N(c3ccc(-c4cccc5c(-c6ccccc6)c(-c6ccccc6)c6ccccc6c45)cc3)c3c(-c4ccccc4)cccc3-c3ccccc3)cc2-c2ccccc2)cc1 ; c1ccc(-c2cccc(-c3ccc(N(c4ccc(-c5cccc6c(-c7ccccc7)c(-c7ccccc7)c7ccccc7c56)cc4)c4c(-c5ccccc5)cccc4-c4ccccc4)cc3)c2-c2ccccc2)cc1]

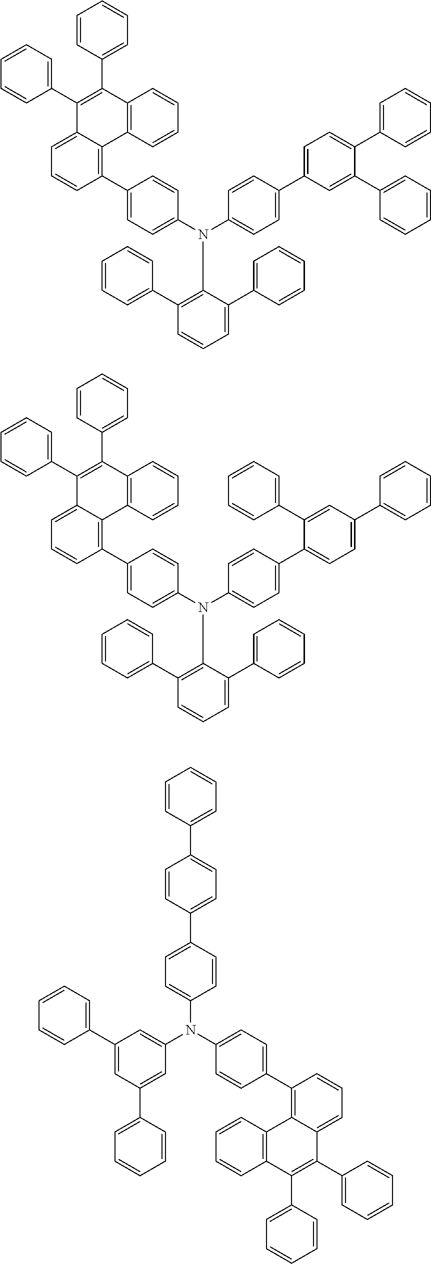 [SMILES: c1ccc(-c2ccc(-c3ccc(N(c4ccc(-c5cccc6c(-c7ccccc7)c(-c7ccccc7)c7ccccc7c56)cc4)c4c(-c5ccccc5)cccc4-c4ccccc4)cc3)cc2-c2ccccc2)cc1 ; c1ccc(-c2ccc(-c3ccc(N(c4ccc(-c5cccc6c(-c7ccccc7)c(-c7ccccc7)c7ccccc7c56)cc4)c4c(-c5ccccc5)cccc4-c4ccccc4)cc3)c(-c3ccccc3)c2)cc1 ; c1ccc(-c2ccc(-c3ccc(N(c4ccc(-c5cccc6c(-c7ccccc7)c(-c7ccccc7)c7ccccc7c56)cc4)c4cc(-c5ccccc5)cc(-c5ccccc5)c4)cc3)cc2)cc1]

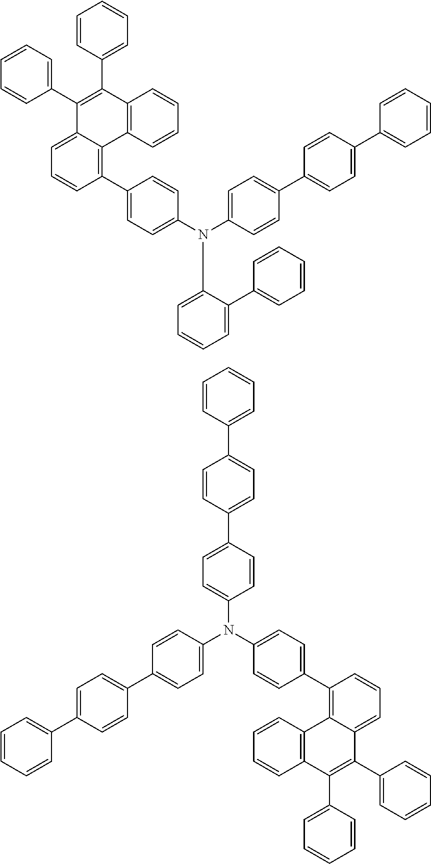

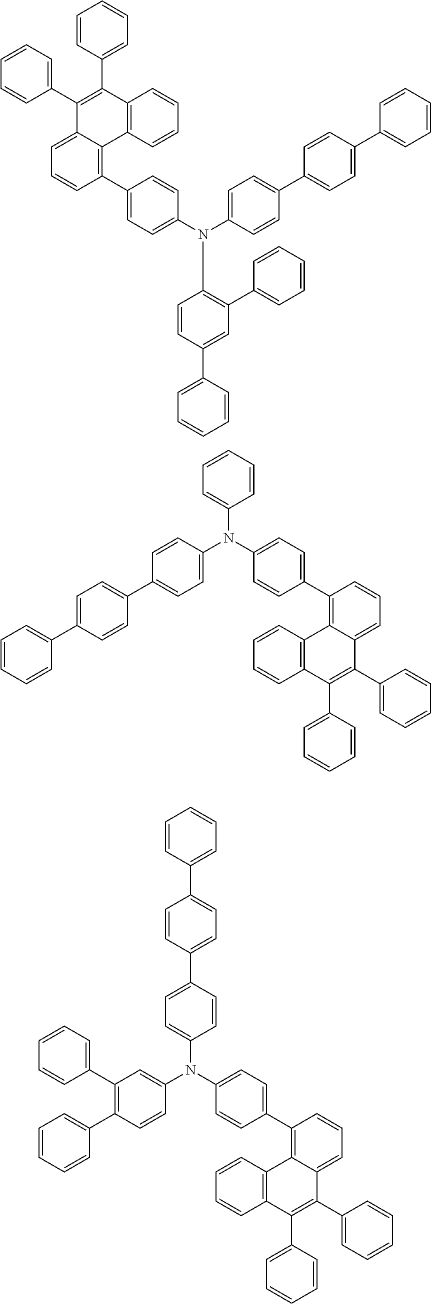

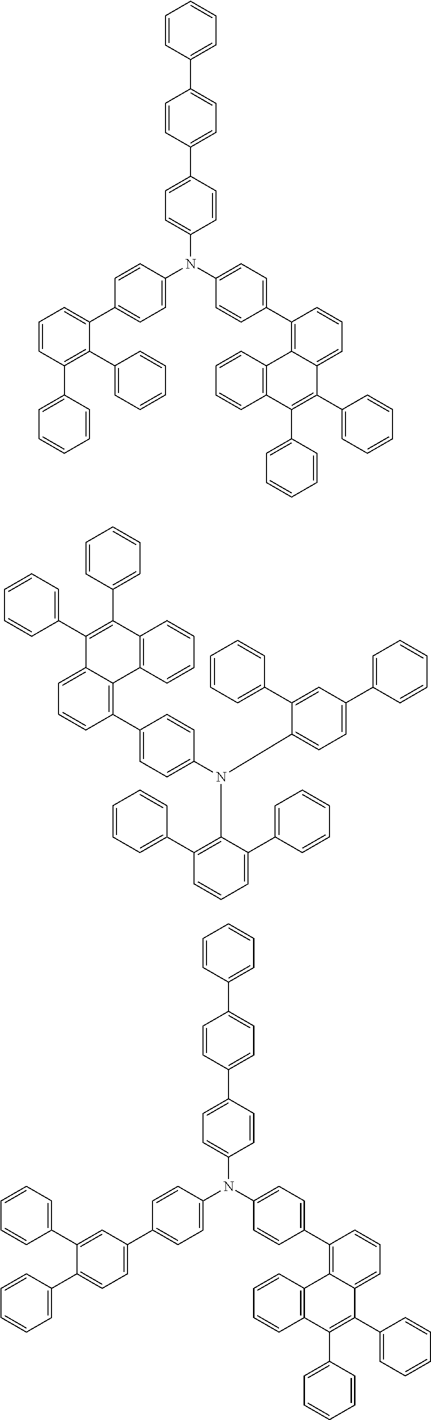

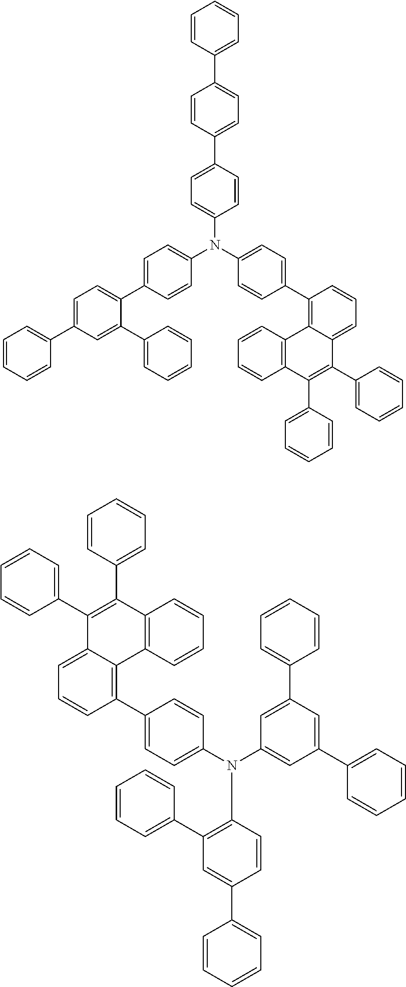 [SMILES: c1ccc(-c2ccc(-c3ccc(N(c4ccc(-c5ccc(-c6ccccc6)cc5-c5ccccc5)cc4)c4ccc(-c5cccc6c(-c7ccccc7)c(-c7ccccc7)c7ccccc7c56)cc4)cc3)cc2)cc1 ; c1ccc(-c2cc(-c3ccccc3)cc(N(c3ccc(-c4cccc5c(-c6ccccc6)c(-c6ccccc6)c6ccccc6c45)cc3)c3ccc(-c4ccccc4)cc3-c3ccccc3)c2)cc1]

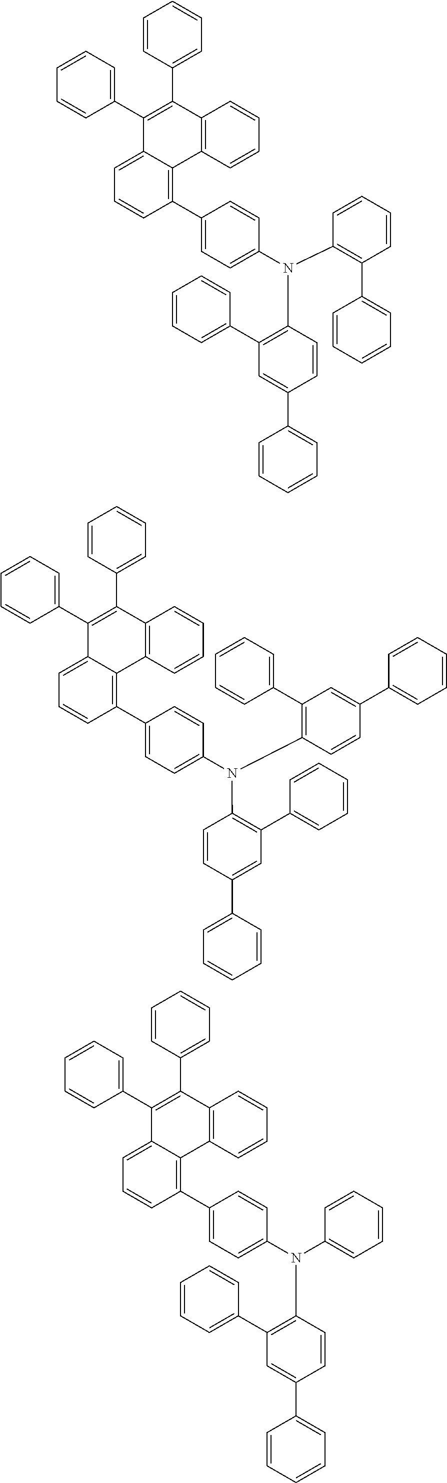

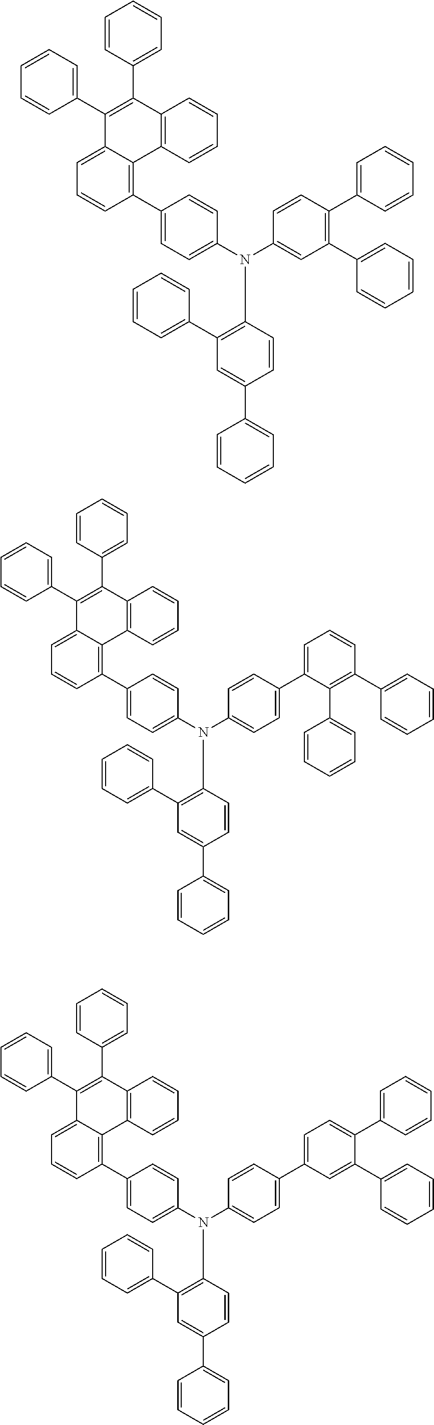 [SMILES: c1ccc(-c2ccc(N(c3ccc(-c4cccc5c(-c6ccccc6)c(-c6ccccc6)c6ccccc6c45)cc3)c3ccc(-c4ccccc4)c(-c4ccccc4)c3)c(-c3ccccc3)c2)cc1 ; c1ccc(-c2ccc(N(c3ccc(-c4cccc(-c5ccccc5)c4-c4ccccc4)cc3)c3ccc(-c4cccc5c(-c6ccccc6)c(-c6ccccc6)c6ccccc6c45)cc3)c(-c3ccccc3)c2)cc1 ; c1ccc(-c2ccc(N(c3ccc(-c4ccc(-c5ccccc5)c(-c5ccccc5)c4)cc3)c3ccc(-c4cccc5c(-c6ccccc6)c(-c6ccccc6)c6ccccc6c45)cc3)c(-c3ccccc3)c2)cc1]

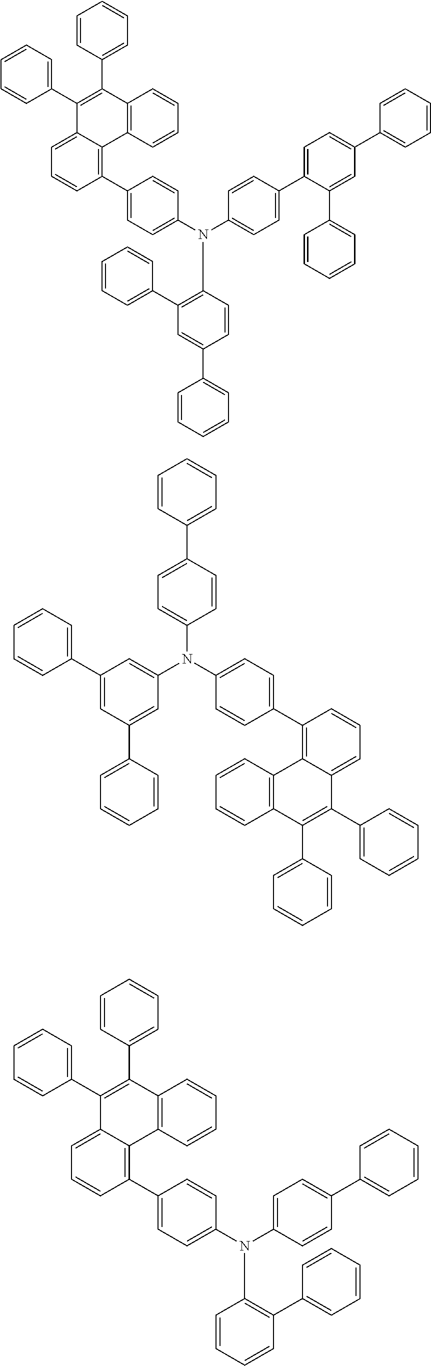

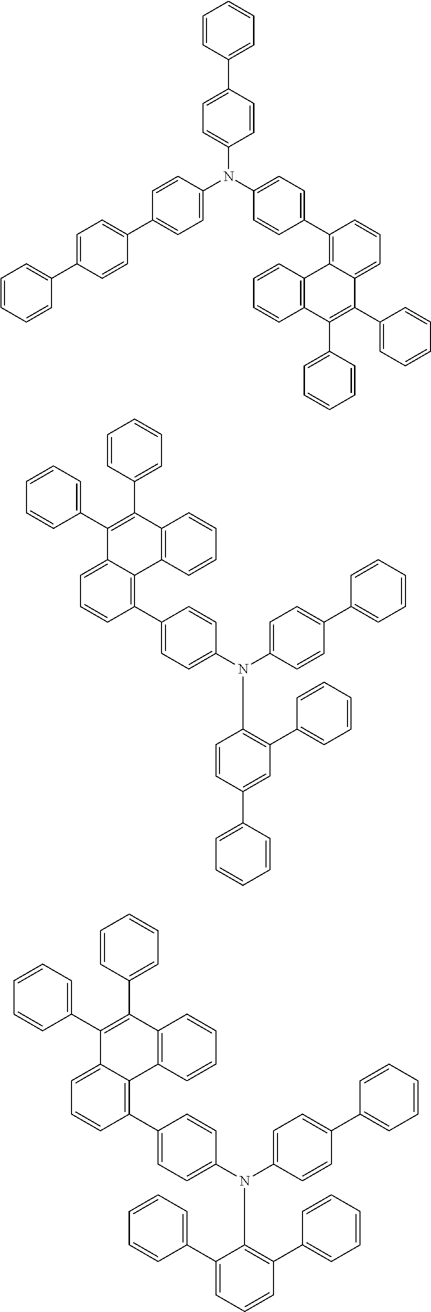 [SMILES: c1ccc(-c2ccc(-c3ccc(N(c4ccc(-c5ccccc5)cc4)c4ccc(-c5cccc6c(-c7ccccc7)c(-c7ccccc7)c7ccccc7c56)cc4)cc3)cc2)cc1 ; c1ccc(-c2ccc(N(c3ccc(-c4cccc5c(-c6ccccc6)c(-c6ccccc6)c6ccccc6c45)cc3)c3ccc(-c4ccccc4)cc3-c3ccccc3)cc2)cc1 ; c1ccc(-c2ccc(N(c3ccc(-c4cccc5c(-c6ccccc6)c(-c6ccccc6)c6ccccc6c45)cc3)c3c(-c4ccccc4)cccc3-c3ccccc3)cc2)cc1]

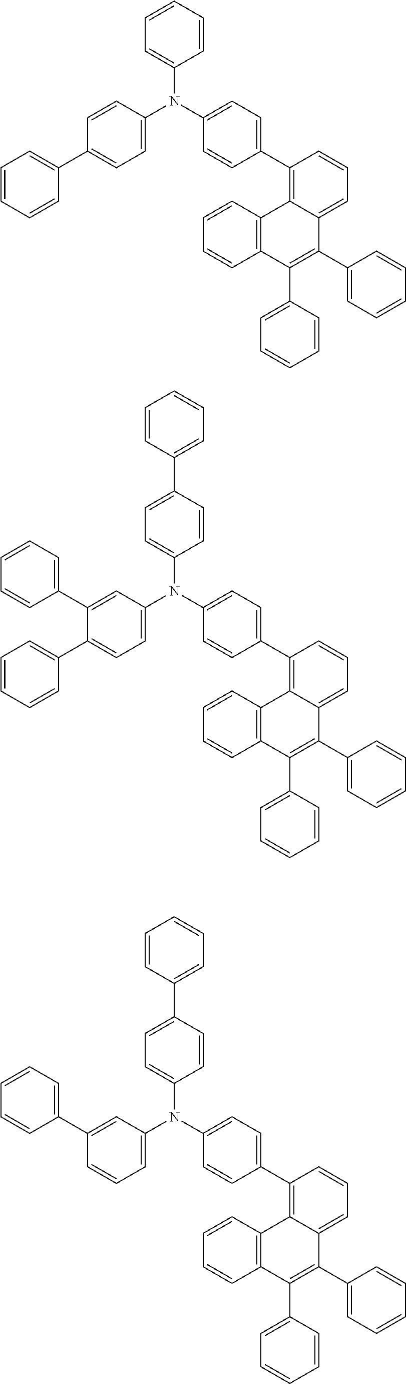 [SMILES: c1ccc(-c2ccc(N(c3ccccc3)c3ccc(-c4cccc5c(-c6ccccc6)c(-c6ccccc6)c6ccccc6c45)cc3)cc2)cc1 ; c1ccc(-c2ccc(N(c3ccc(-c4cccc5c(-c6ccccc6)c(-c6ccccc6)c6ccccc6c45)cc3)c3ccc(-c4ccccc4)c(-c4ccccc4)c3)cc2)cc1 ; c1ccc(-c2ccc(N(c3ccc(-c4cccc5c(-c6ccccc6)c(-c6ccccc6)c6ccccc6c45)cc3)c3cccc(-c4ccccc4)c3)cc2)cc1]

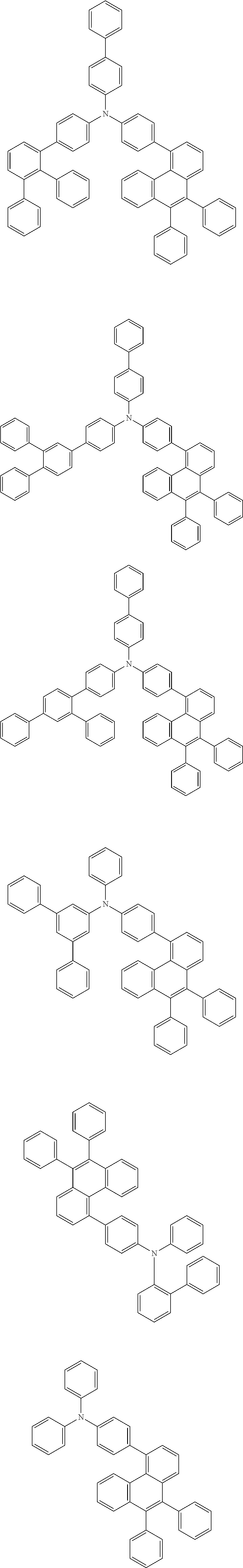

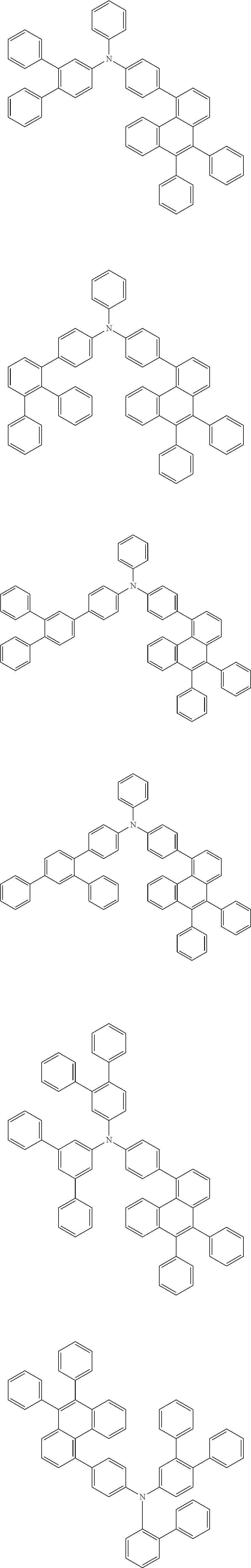

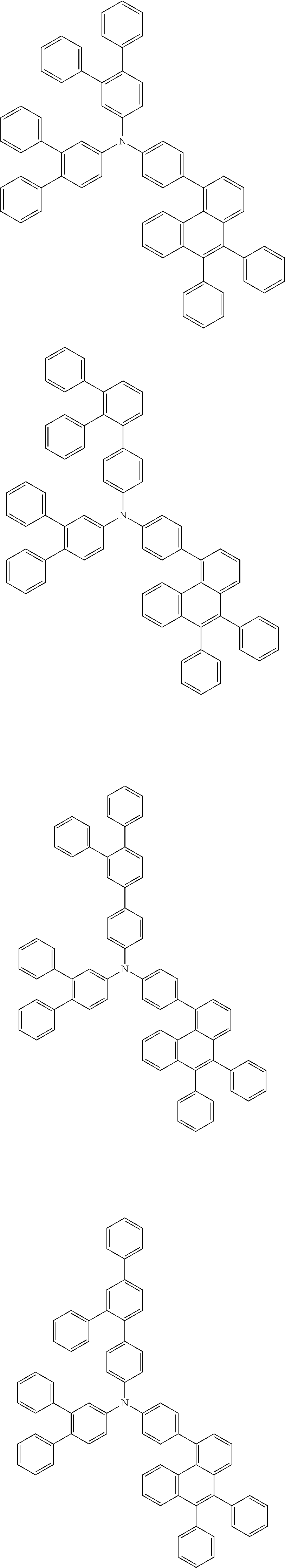

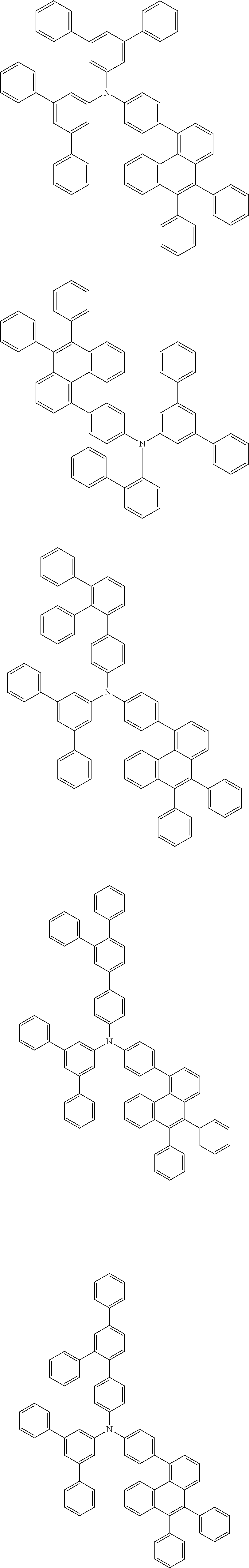

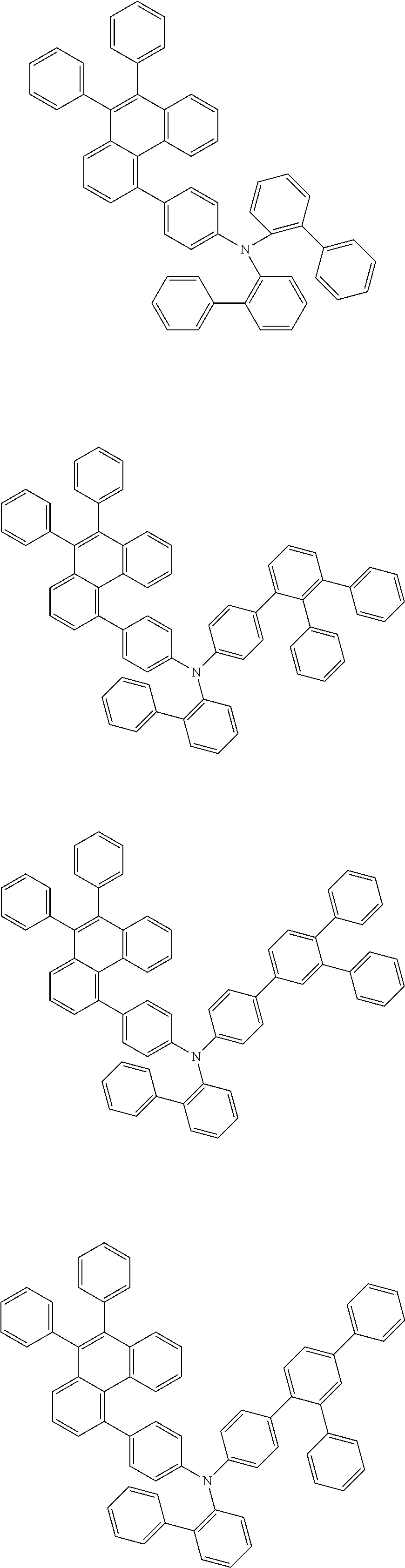

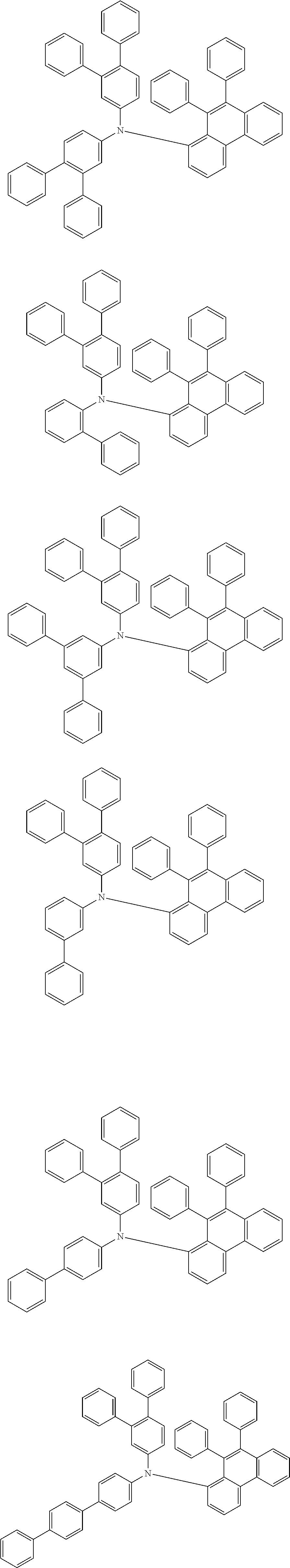

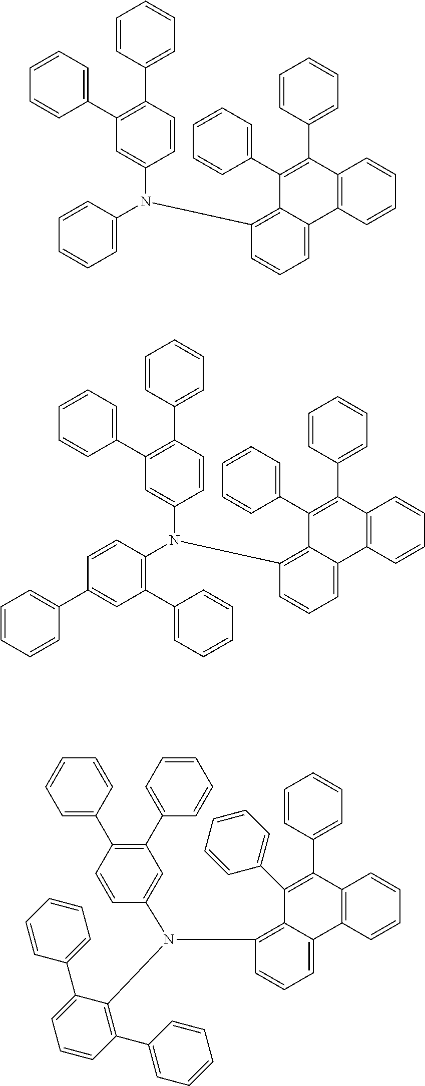 [SMILES: c1ccc(-c2ccc(N(c3ccccc3)c3cccc4c3c(-c3ccccc3)c(-c3ccccc3)c3ccccc34)cc2-c2ccccc2)cc1 ; c1ccc(-c2ccc(N(c3ccc(-c4ccccc4)c(-c4ccccc4)c3)c3cccc4c3c(-c3ccccc3)c(-c3ccccc3)c3ccccc34)c(-c3ccccc3)c2)cc1 ; c1ccc(-c2ccc(N(c3c(-c4ccccc4)cccc3-c3ccccc3)c3cccc4c3c(-c3ccccc3)c(-c3ccccc3)c3ccccc34)cc2-c2ccccc2)cc1]

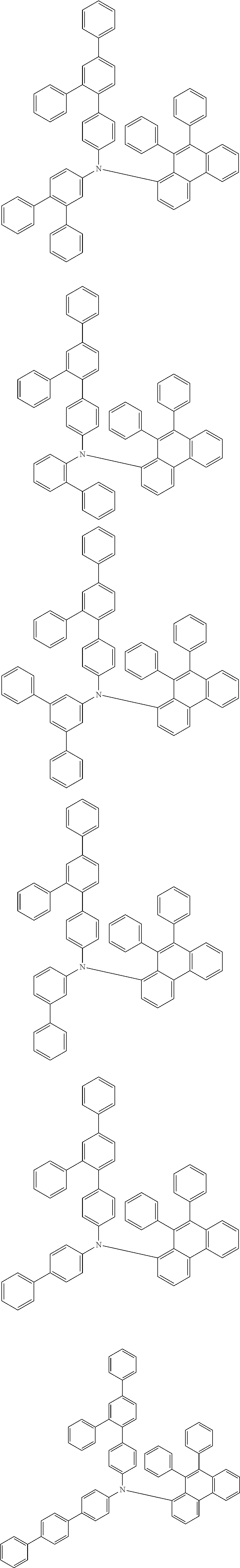

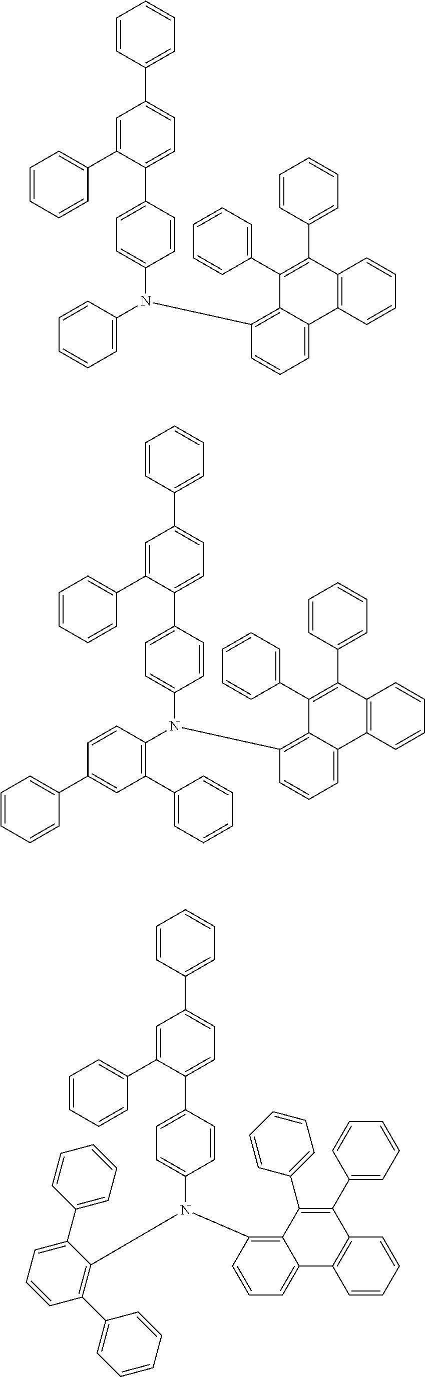 [SMILES: c1ccc(-c2ccc(-c3ccc(N(c4ccccc4)c4cccc5c4c(-c4ccccc4)c(-c4ccccc4)c4ccccc45)cc3)c(-c3ccccc3)c2)cc1 ; c1ccc(-c2ccc(-c3ccc(N(c4ccc(-c5ccccc5)cc4-c4ccccc4)c4cccc5c4c(-c4ccccc4)c(-c4ccccc4)c4ccccc45)cc3)c(-c3ccccc3)c2)cc1 ; c1ccc(-c2ccc(-c3ccc(N(c4c(-c5ccccc5)cccc4-c4ccccc4)c4cccc5c4c(-c4ccccc4)c(-c4ccccc4)c4ccccc45)cc3)c(-c3ccccc3)c2)cc1]

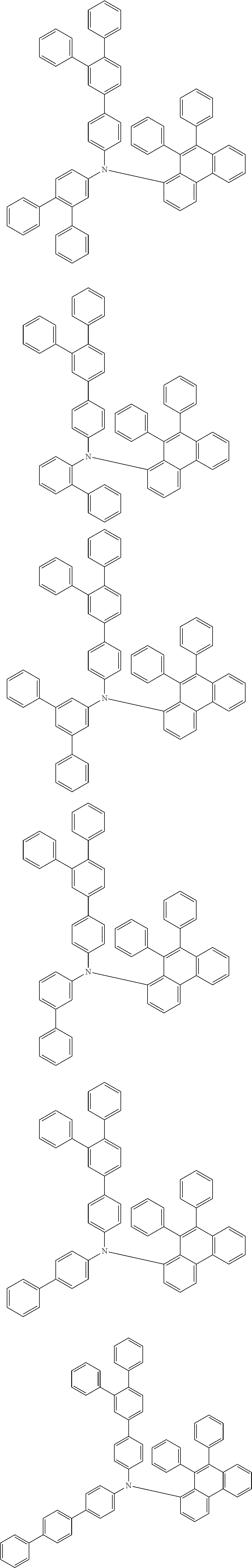

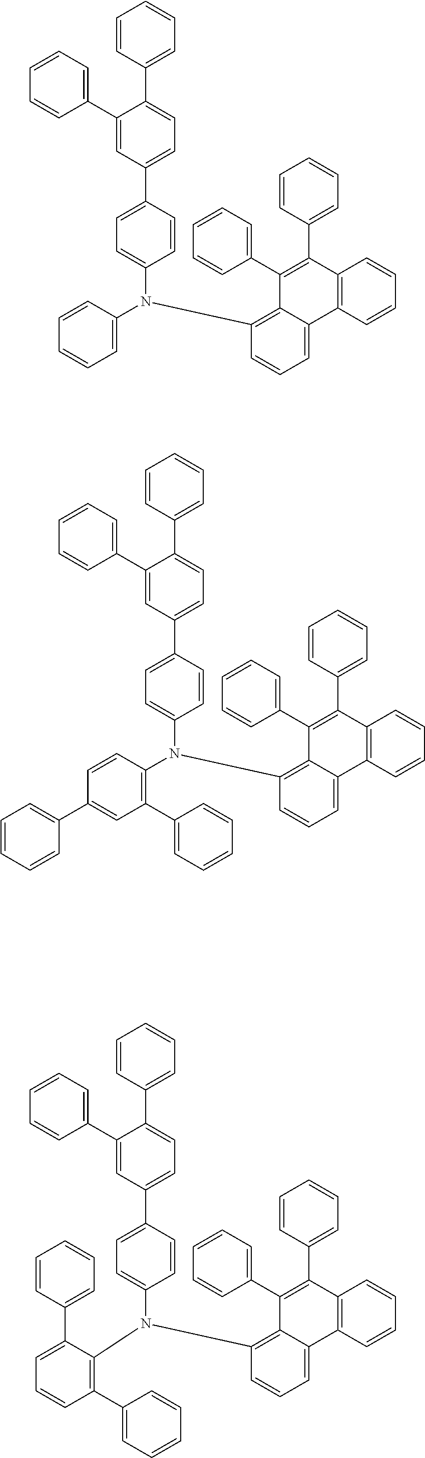 [SMILES: c1ccc(-c2ccc(-c3ccc(N(c4ccccc4)c4cccc5c4c(-c4ccccc4)c(-c4ccccc4)c4ccccc45)cc3)cc2-c2ccccc2)cc1 ; c1ccc(-c2ccc(N(c3ccc(-c4ccc(-c5ccccc5)c(-c5ccccc5)c4)cc3)c3cccc4c3c(-c3ccccc3)c(-c3ccccc3)c3ccccc34)c(-c3ccccc3)c2)cc1 ; c1ccc(-c2ccc(-c3ccc(N(c4c(-c5ccccc5)cccc4-c4ccccc4)c4cccc5c4c(-c4ccccc4)c(-c4ccccc4)c4ccccc45)cc3)cc2-c2ccccc2)cc1]

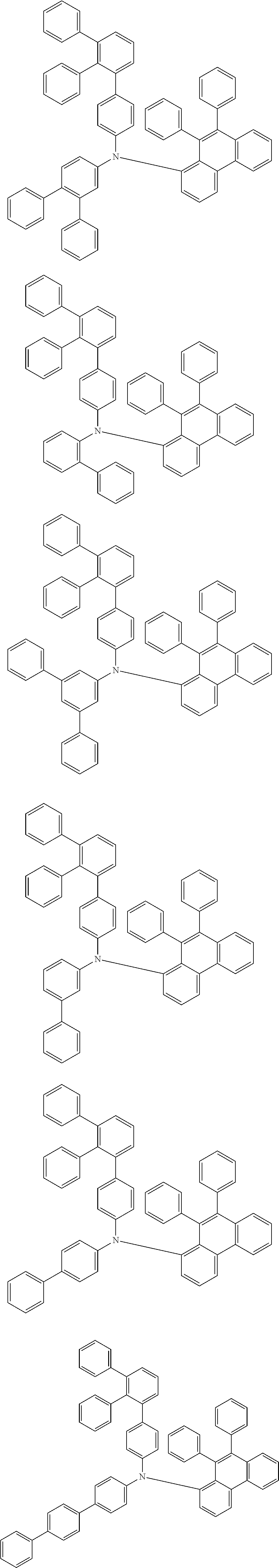

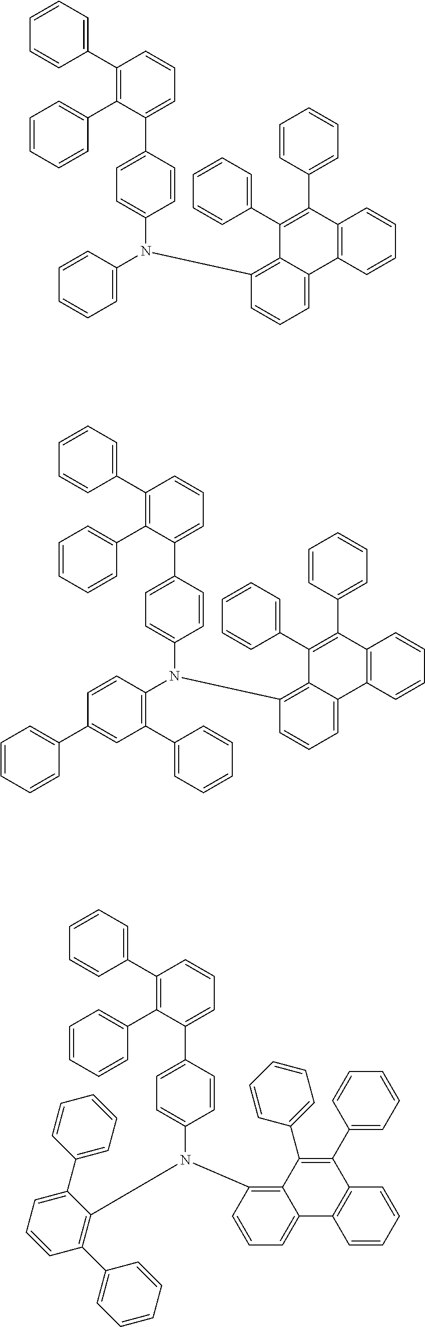 [SMILES: c1ccc(-c2cccc(-c3ccc(N(c4ccccc4)c4cccc5c4c(-c4ccccc4)c(-c4ccccc4)c4ccccc45)cc3)c2-c2ccccc2)cc1 ; c1ccc(-c2ccc(N(c3ccc(-c4cccc(-c5ccccc5)c4-c4ccccc4)cc3)c3cccc4c3c(-c3ccccc3)c(-c3ccccc3)c3ccccc34)c(-c3ccccc3)c2)cc1 ; c1ccc(-c2cccc(-c3ccc(N(c4c(-c5ccccc5)cccc4-c4ccccc4)c4cccc5c4c(-c4ccccc4)c(-c4ccccc4)c4ccccc45)cc3)c2-c2ccccc2)cc1]

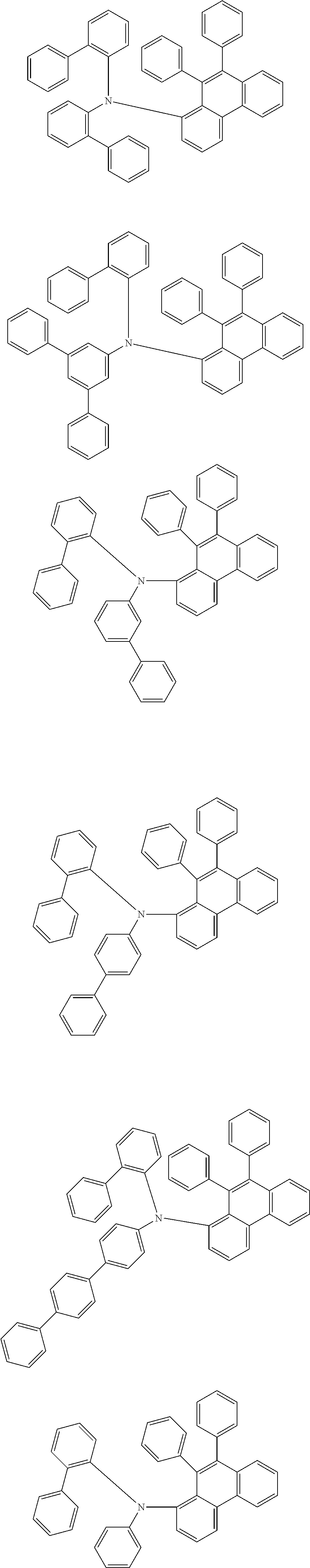

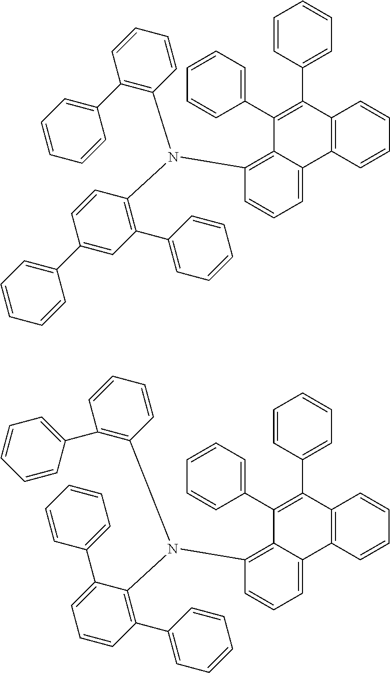 [SMILES: c1ccc(-c2ccc(N(c3ccccc3-c3ccccc3)c3cccc4c3c(-c3ccccc3)c(-c3ccccc3)c3ccccc34)c(-c3ccccc3)c2)cc1 ; c1ccc(-c2ccccc2N(c2c(-c3ccccc3)cccc2-c2ccccc2)c2cccc3c2c(-c2ccccc2)c(-c2ccccc2)c2ccccc23)cc1]

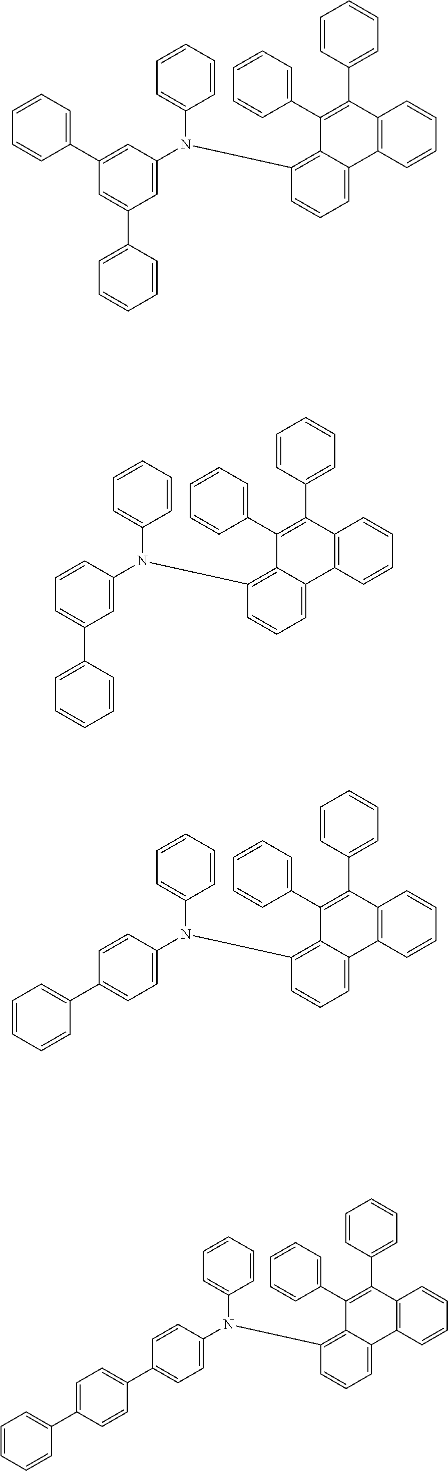 [SMILES: c1ccc(-c2cc(-c3ccccc3)cc(N(c3ccccc3)c3cccc4c3c(-c3ccccc3)c(-c3ccccc3)c3ccccc34)c2)cc1 ; c1ccc(-c2cccc(N(c3ccccc3)c3cccc4c3c(-c3ccccc3)c(-c3ccccc3)c3ccccc34)c2)cc1 ; c1ccc(-c2ccc(N(c3ccccc3)c3cccc4c3c(-c3ccccc3)c(-c3ccccc3)c3ccccc34)cc2)cc1 ; c1ccc(-c2ccc(-c3ccc(N(c4ccccc4)c4cccc5c4c(-c4ccccc4)c(-c4ccccc4)c4ccccc45)cc3)cc2)cc1]

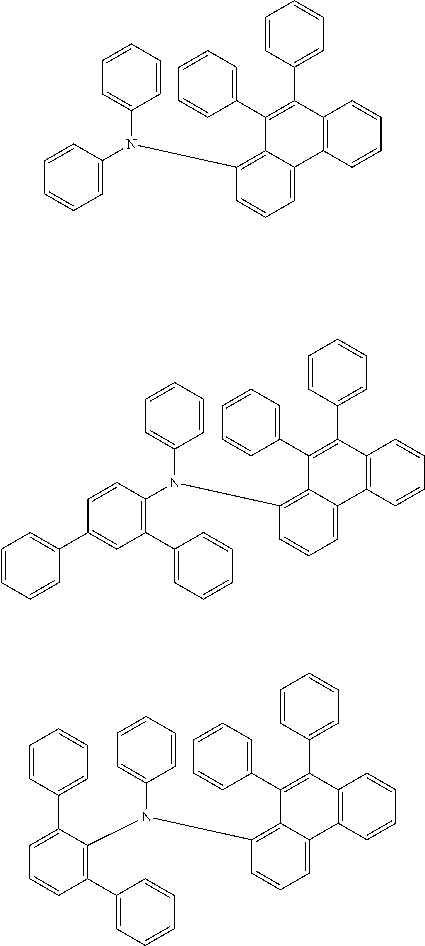 [SMILES: c1ccc(-c2c(-c3ccccc3)c3c(N(c4ccccc4)c4ccccc4)cccc3c3ccccc23)cc1 ; c1ccc(-c2ccc(N(c3ccccc3)c3cccc4c3c(-c3ccccc3)c(-c3ccccc3)c3ccccc34)c(-c3ccccc3)c2)cc1 ; c1ccc(-c2cccc(-c3ccccc3)c2N(c2ccccc2)c2cccc3c2c(-c2ccccc2)c(-c2ccccc2)c2ccccc23)cc1]

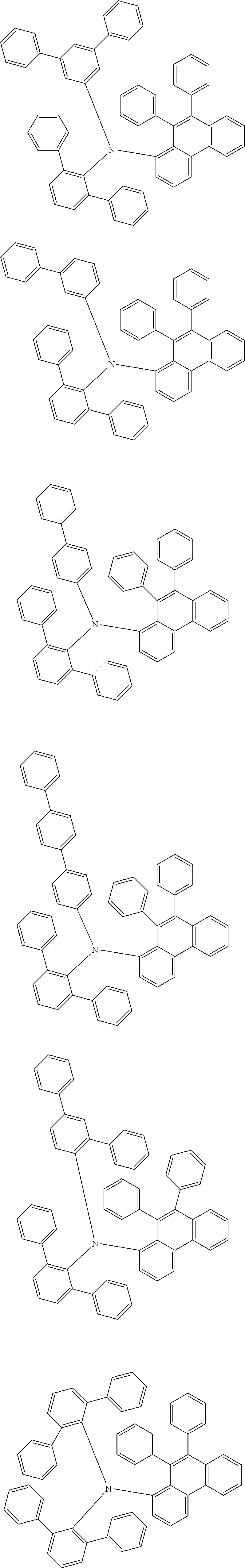

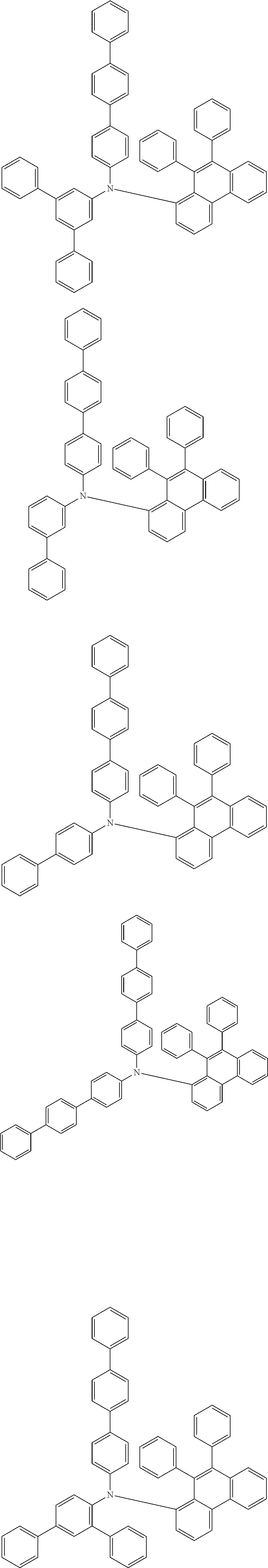

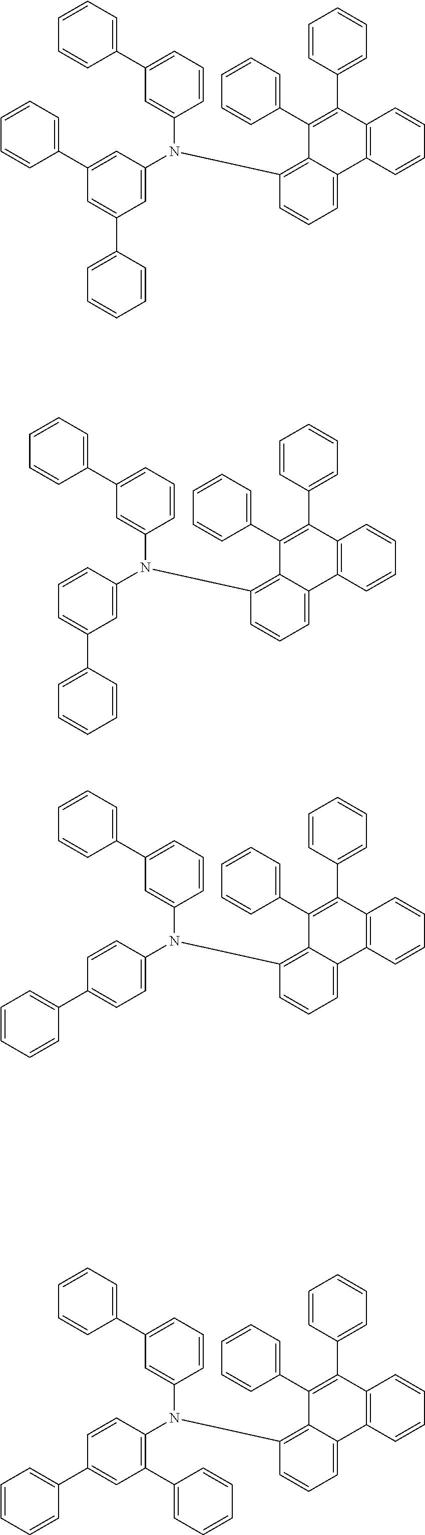 [SMILES: c1ccc(-c2cccc(N(c3cc(-c4ccccc4)cc(-c4ccccc4)c3)c3cccc4c3c(-c3ccccc3)c(-c3ccccc3)c3ccccc34)c2)cc1 ; c1ccc(-c2cccc(N(c3cccc(-c4ccccc4)c3)c3cccc4c3c(-c3ccccc3)c(-c3ccccc3)c3ccccc34)c2)cc1 ; c1ccc(-c2ccc(N(c3cccc(-c4ccccc4)c3)c3cccc4c3c(-c3ccccc3)c(-c3ccccc3)c3ccccc34)cc2)cc1 ; c1ccc(-c2cccc(N(c3ccc(-c4ccccc4)cc3-c3ccccc3)c3cccc4c3c(-c3ccccc3)c(-c3ccccc3)c3ccccc34)c2)cc1]

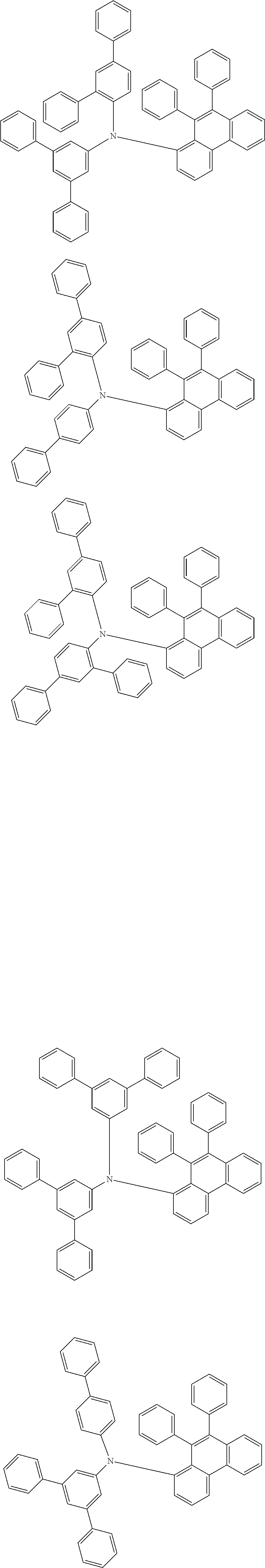

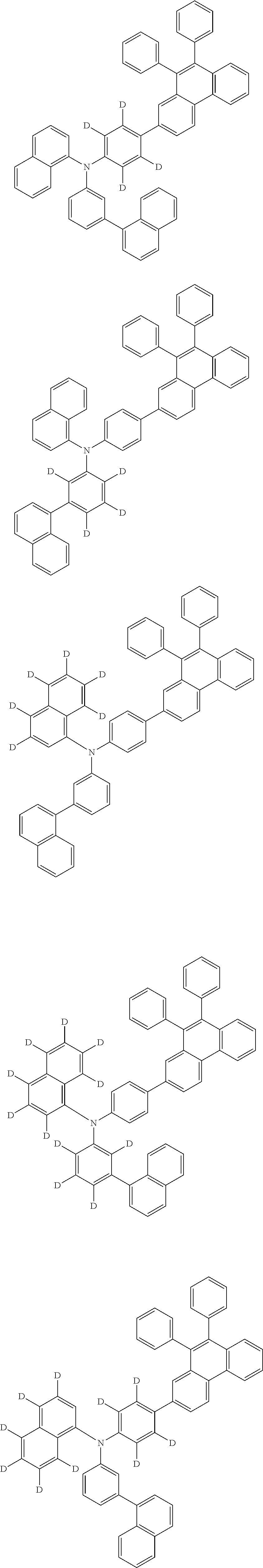

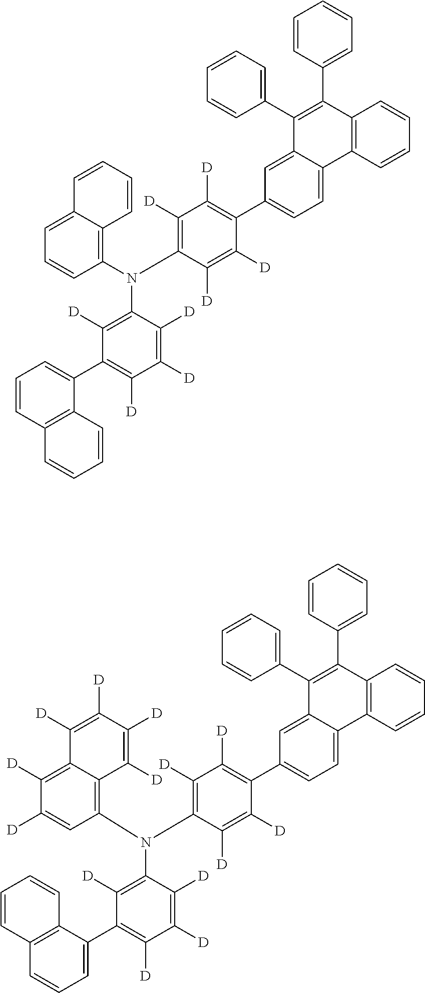 [SMILES: [2H]c1c([2H])c(-c2cccc3ccccc23)c([2H])c(N(c2c([2H])c([2H])c(-c3ccc4c(c3)c(-c3ccccc3)c(-c3ccccc3)c3ccccc34)c([2H])c2[2H])c2cccc3ccccc23)c1[2H] ; [2H]c1cc(N(c2c([2H])c([2H])c(-c3ccc4c(c3)c(-c3ccccc3)c(-c3ccccc3)c3ccccc34)c([2H])c2[2H])c2c([2H])c([2H])c([2H])c(-c3cccc4ccccc34)c2[2H])c2c([2H])c([2H])c([2H])c([2H])c2c1[2H]]

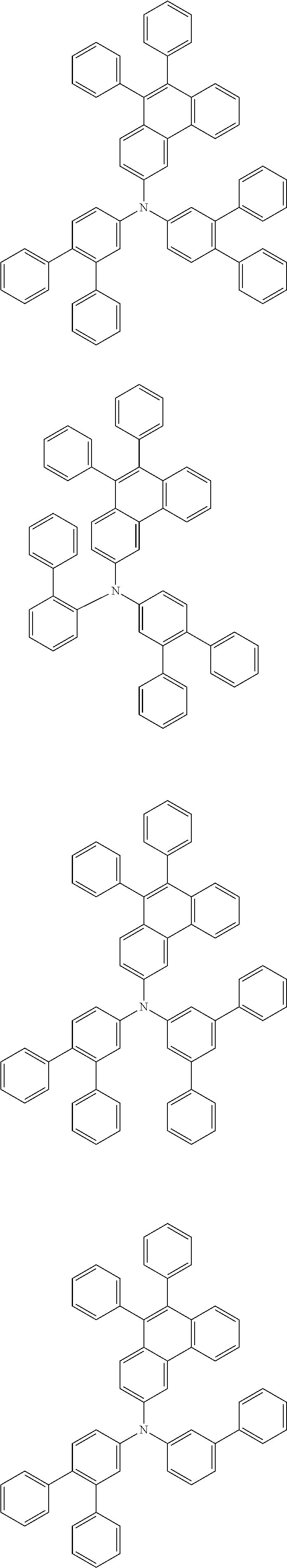 [SMILES: c1ccc(-c2ccc(N(c3ccc(-c4ccccc4)c(-c4ccccc4)c3)c3ccc4c(-c5ccccc5)c(-c5ccccc5)c5ccccc5c4c3)cc2-c2ccccc2)cc1 ; c1ccc(-c2ccc(N(c3ccc4c(-c5ccccc5)c(-c5ccccc5)c5ccccc5c4c3)c3ccccc3-c3ccccc3)cc2-c2ccccc2)cc1 ; c1ccc(-c2cc(-c3ccccc3)cc(N(c3ccc(-c4ccccc4)c(-c4ccccc4)c3)c3ccc4c(-c5ccccc5)c(-c5ccccc5)c5ccccc5c4c3)c2)cc1 ; c1ccc(-c2cccc(N(c3ccc(-c4ccccc4)c(-c4ccccc4)c3)c3ccc4c(-c5ccccc5)c(-c5ccccc5)c5ccccc5c4c3)c2)cc1]

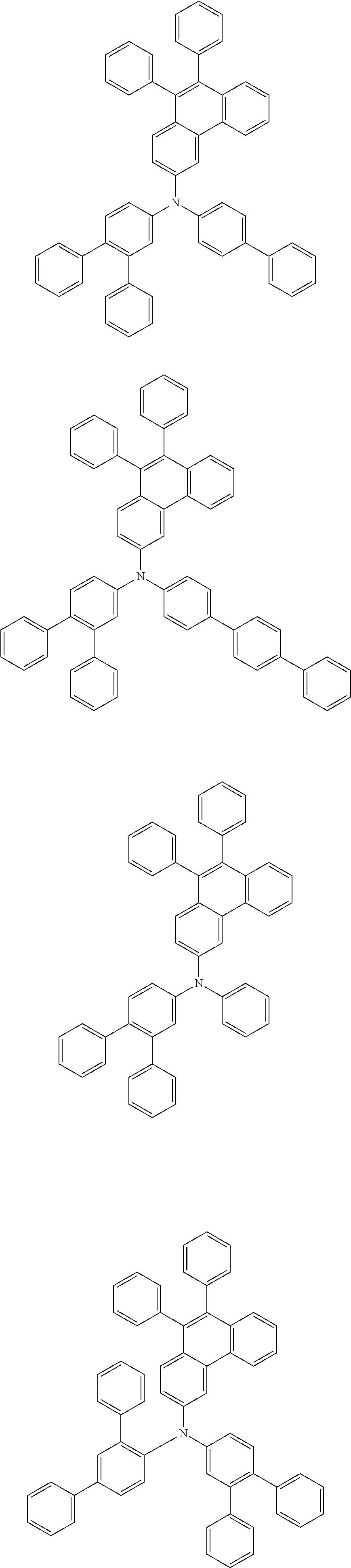 [SMILES: c1ccc(-c2ccc(N(c3ccc(-c4ccccc4)c(-c4ccccc4)c3)c3ccc4c(-c5ccccc5)c(-c5ccccc5)c5ccccc5c4c3)cc2)cc1 ; c1ccc(-c2ccc(-c3ccc(N(c4ccc(-c5ccccc5)c(-c5ccccc5)c4)c4ccc5c(-c6ccccc6)c(-c6ccccc6)c6ccccc6c5c4)cc3)cc2)cc1 ; c1ccc(-c2ccc(N(c3ccccc3)c3ccc4c(-c5ccccc5)c(-c5ccccc5)c5ccccc5c4c3)cc2-c2ccccc2)cc1 ; c1ccc(-c2ccc(N(c3ccc(-c4ccccc4)c(-c4ccccc4)c3)c3ccc4c(-c5ccccc5)c(-c5ccccc5)c5ccccc5c4c3)c(-c3ccccc3)c2)cc1]

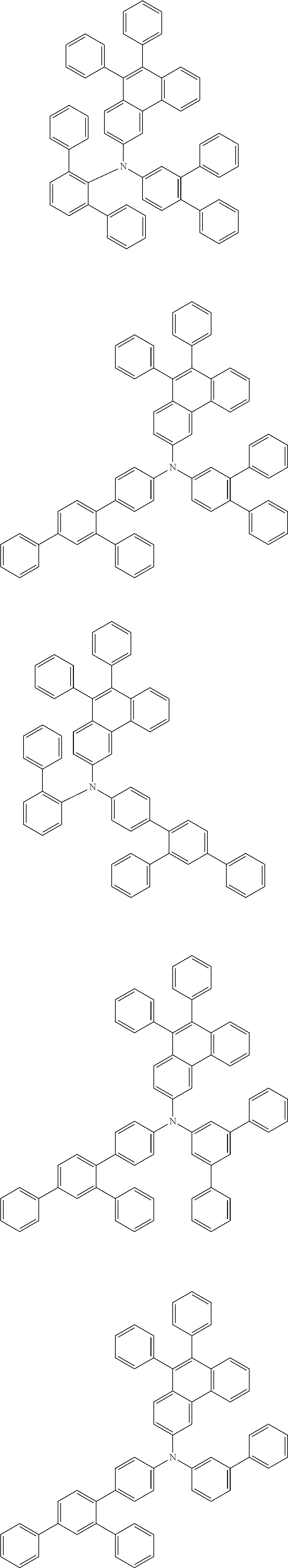

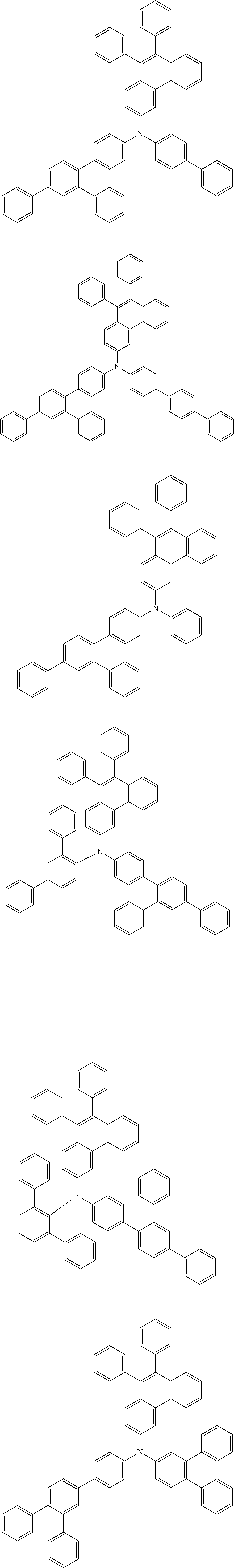

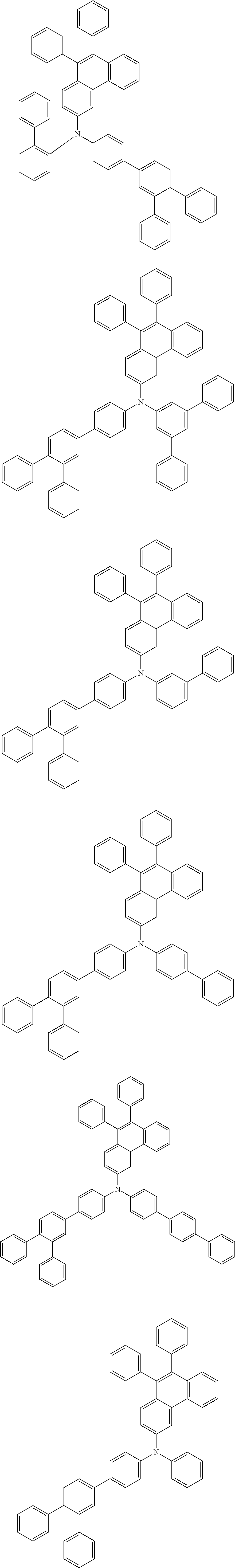

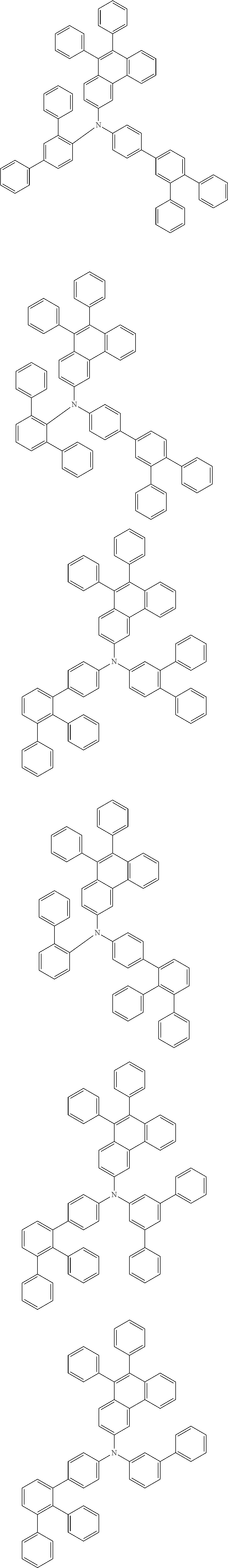

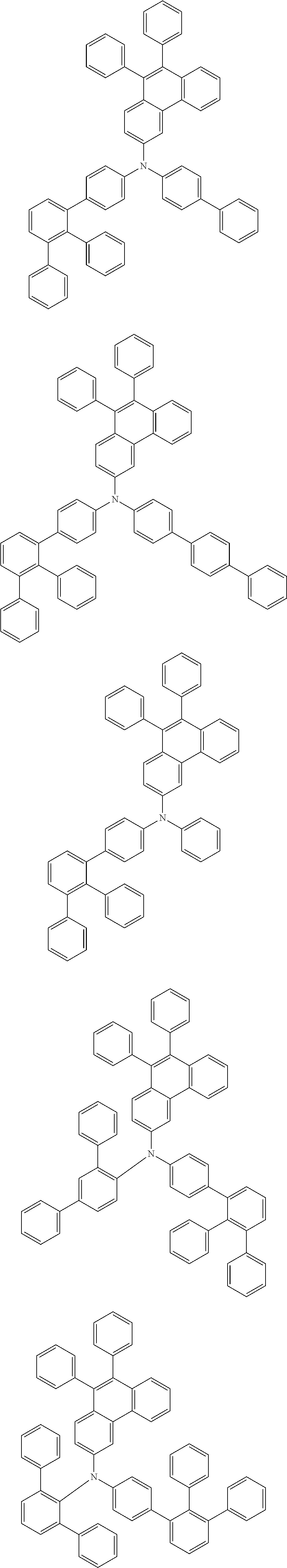

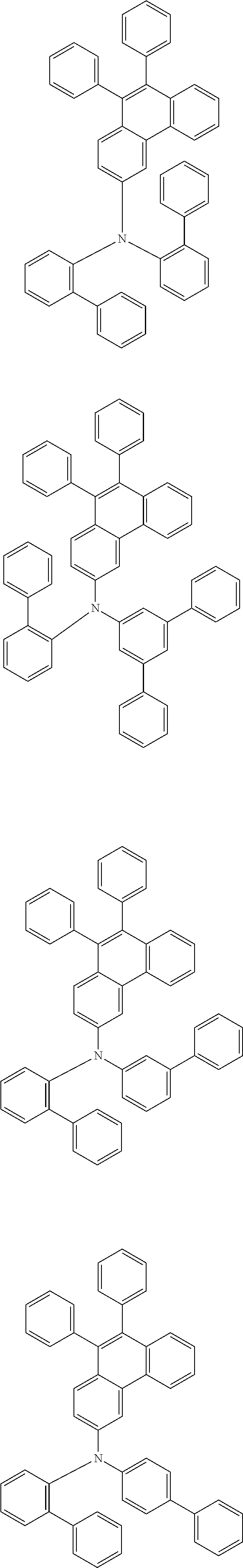 [SMILES: c1ccc(-c2ccccc2N(c2ccc3c(-c4ccccc4)c(-c4ccccc4)c4ccccc4c3c2)c2ccccc2-c2ccccc2)cc1 ; c1ccc(-c2cc(-c3ccccc3)cc(N(c3ccc4c(-c5ccccc5)c(-c5ccccc5)c5ccccc5c4c3)c3ccccc3-c3ccccc3)c2)cc1 ; c1ccc(-c2cccc(N(c3ccc4c(-c5ccccc5)c(-c5ccccc5)c5ccccc5c4c3)c3ccccc3-c3ccccc3)c2)cc1 ; c1ccc(-c2ccc(N(c3ccc4c(-c5ccccc5)c(-c5ccccc5)c5ccccc5c4c3)c3ccccc3-c3ccccc3)cc2)cc1]

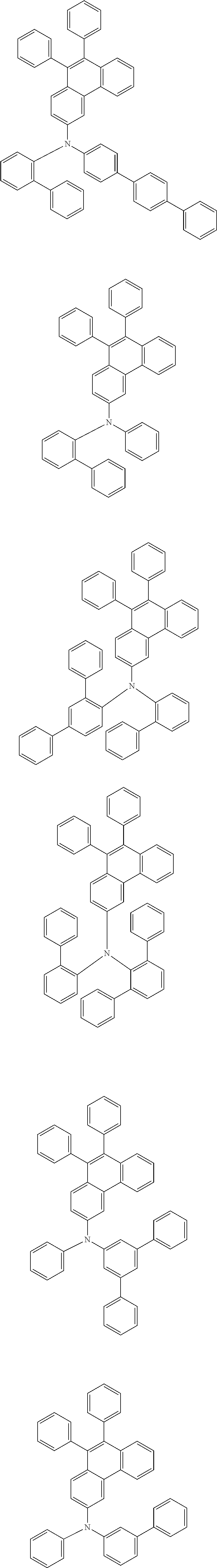

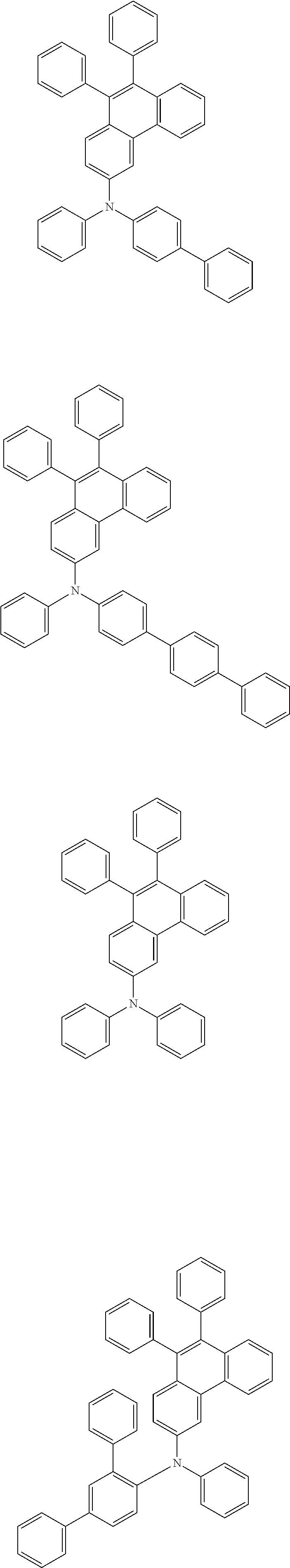 [SMILES: c1ccc(-c2ccc(N(c3ccccc3)c3ccc4c(-c5ccccc5)c(-c5ccccc5)c5ccccc5c4c3)cc2)cc1 ; c1ccc(-c2ccc(-c3ccc(N(c4ccccc4)c4ccc5c(-c6ccccc6)c(-c6ccccc6)c6ccccc6c5c4)cc3)cc2)cc1 ; c1ccc(-c2c(-c3ccccc3)c3ccc(N(c4ccccc4)c4ccccc4)cc3c3ccccc23)cc1 ; c1ccc(-c2ccc(N(c3ccccc3)c3ccc4c(-c5ccccc5)c(-c5ccccc5)c5ccccc5c4c3)c(-c3ccccc3)c2)cc1]

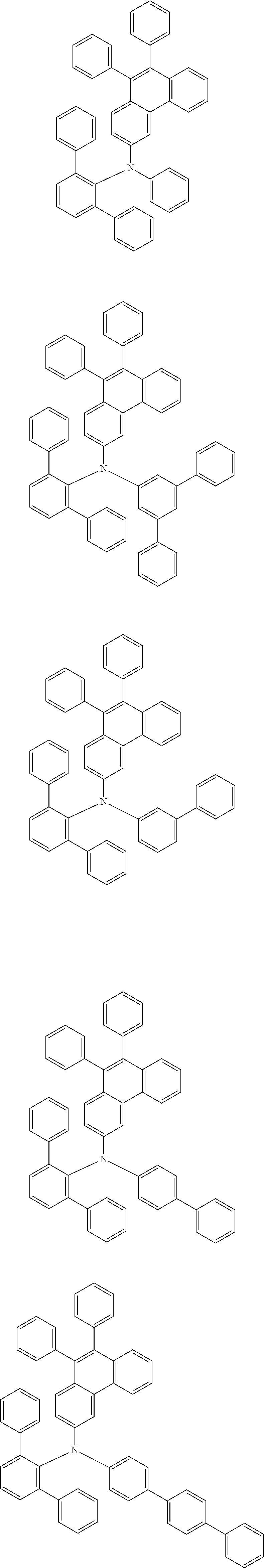 [SMILES: c1ccc(-c2cccc(-c3ccccc3)c2N(c2ccccc2)c2ccc3c(-c4ccccc4)c(-c4ccccc4)c4ccccc4c3c2)cc1 ; c1ccc(-c2cc(-c3ccccc3)cc(N(c3ccc4c(-c5ccccc5)c(-c5ccccc5)c5ccccc5c4c3)c3c(-c4ccccc4)cccc3-c3ccccc3)c2)cc1 ; c1ccc(-c2cccc(N(c3ccc4c(-c5ccccc5)c(-c5ccccc5)c5ccccc5c4c3)c3c(-c4ccccc4)cccc3-c3ccccc3)c2)cc1 ; c1ccc(-c2ccc(N(c3ccc4c(-c5ccccc5)c(-c5ccccc5)c5ccccc5c4c3)c3c(-c4ccccc4)cccc3-c3ccccc3)cc2)cc1 ; c1ccc(-c2ccc(-c3ccc(N(c4ccc5c(-c6ccccc6)c(-c6ccccc6)c6ccccc6c5c4)c4c(-c5ccccc5)cccc4-c4ccccc4)cc3)cc2)cc1]

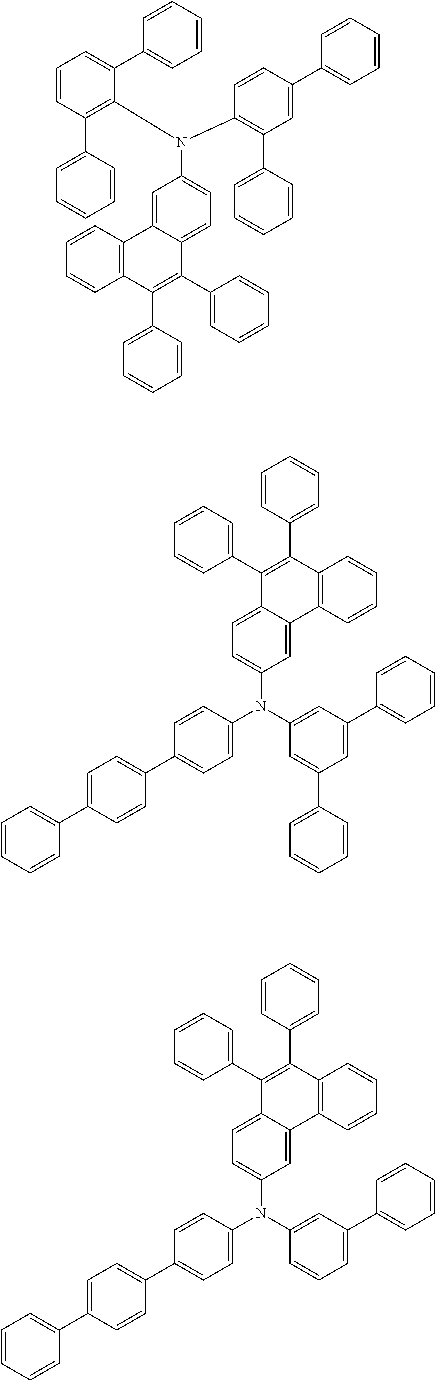 [SMILES: c1ccc(-c2ccc(N(c3ccc4c(-c5ccccc5)c(-c5ccccc5)c5ccccc5c4c3)c3c(-c4ccccc4)cccc3-c3ccccc3)c(-c3ccccc3)c2)cc1 ; c1ccc(-c2ccc(-c3ccc(N(c4cc(-c5ccccc5)cc(-c5ccccc5)c4)c4ccc5c(-c6ccccc6)c(-c6ccccc6)c6ccccc6c5c4)cc3)cc2)cc1 ; c1ccc(-c2ccc(-c3ccc(N(c4cccc(-c5ccccc5)c4)c4ccc5c(-c6ccccc6)c(-c6ccccc6)c6ccccc6c5c4)cc3)cc2)cc1]

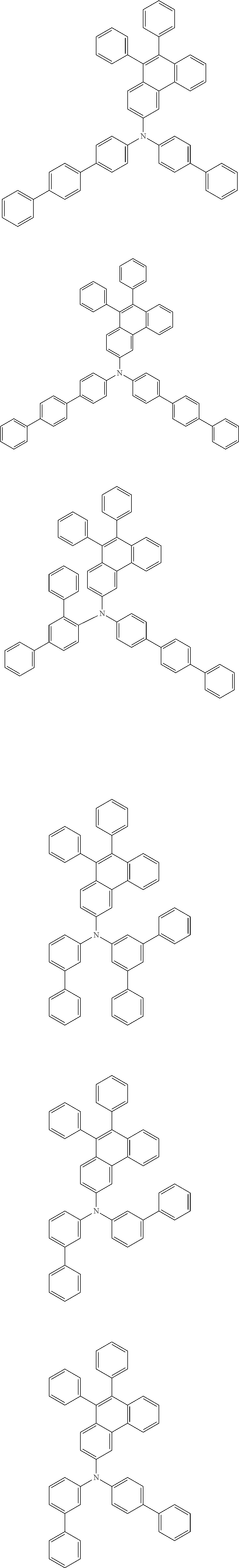

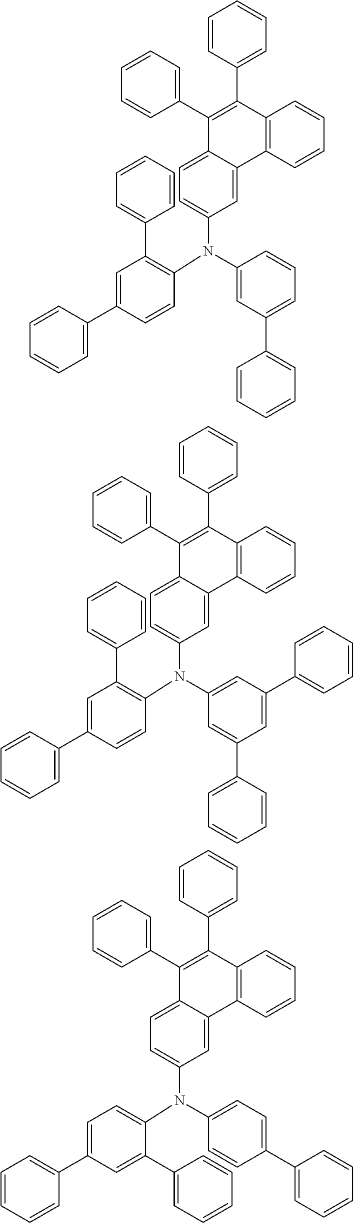

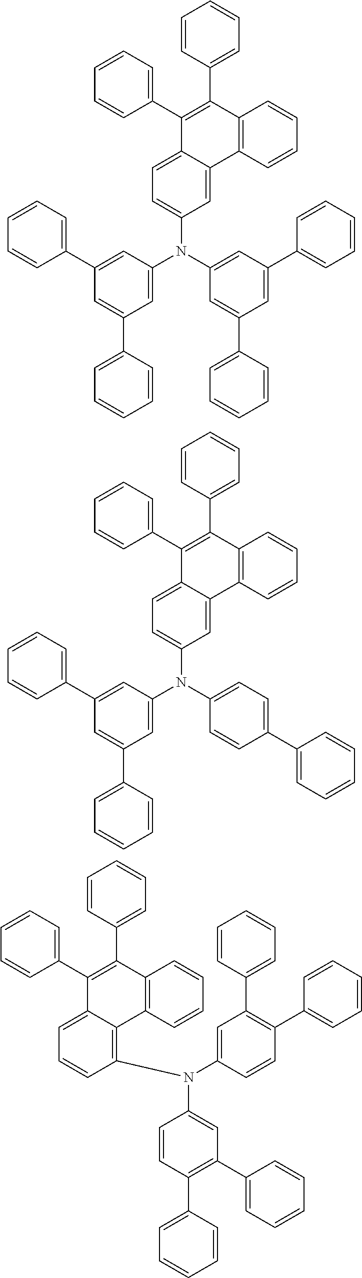

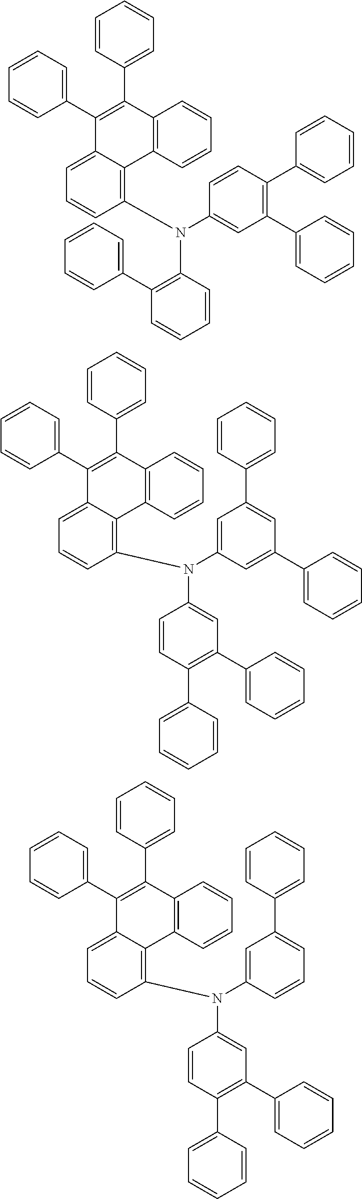

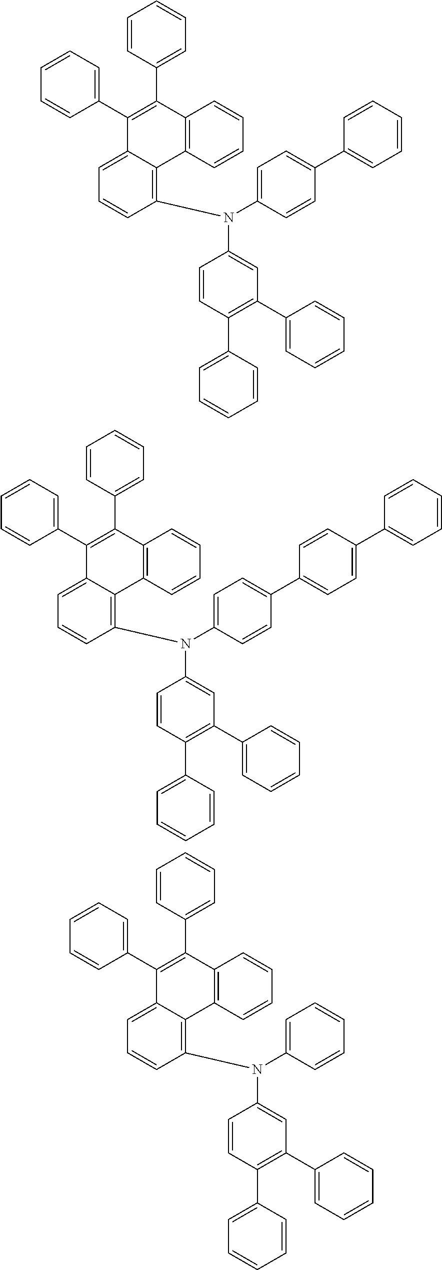

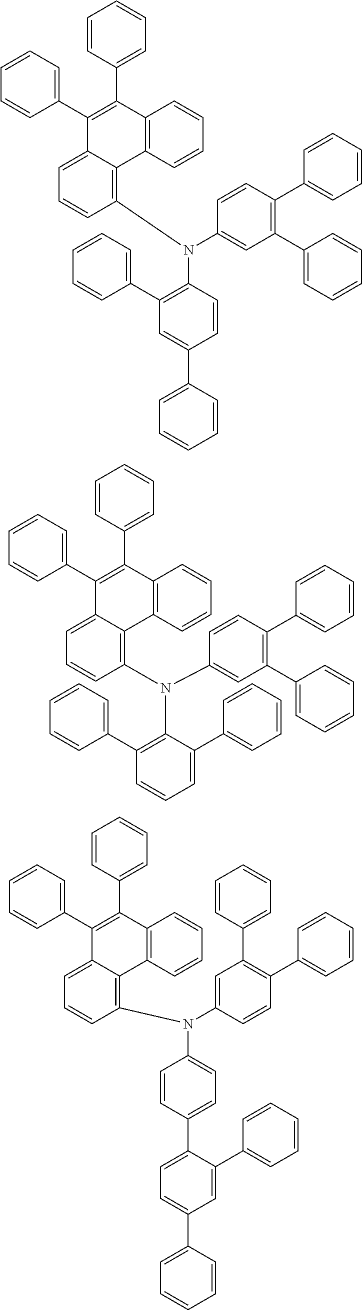

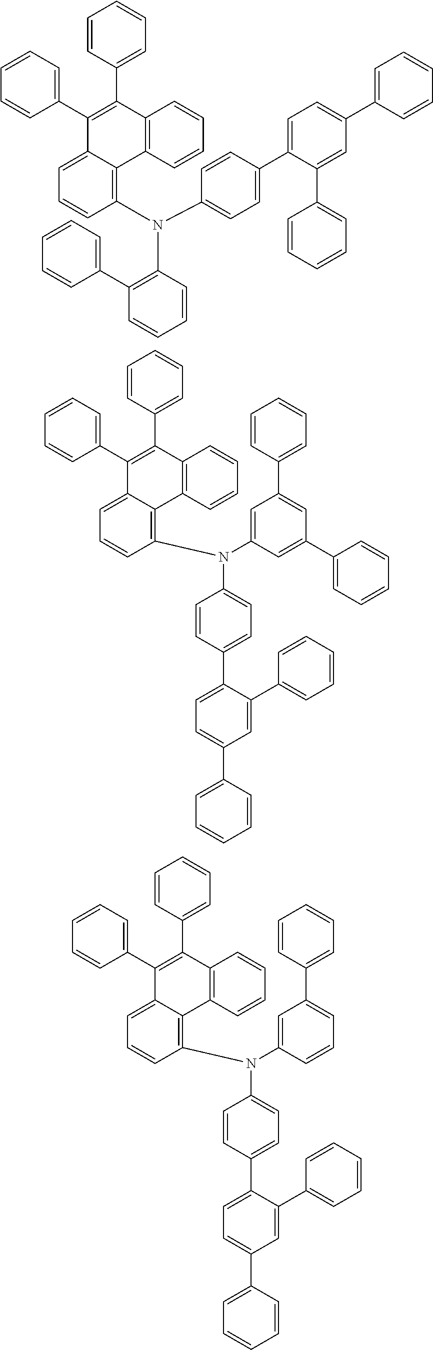

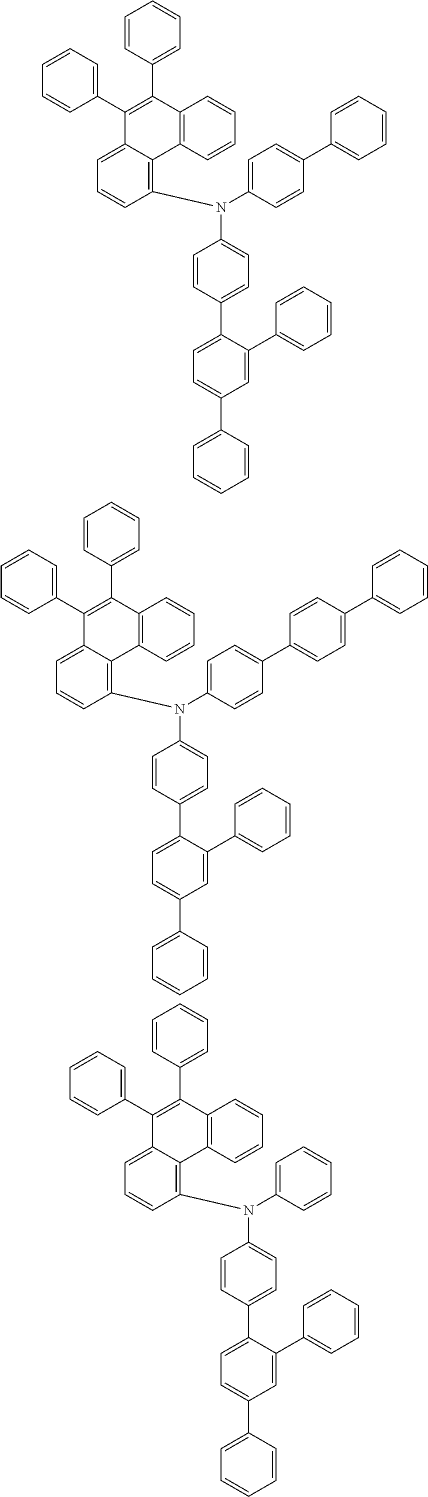

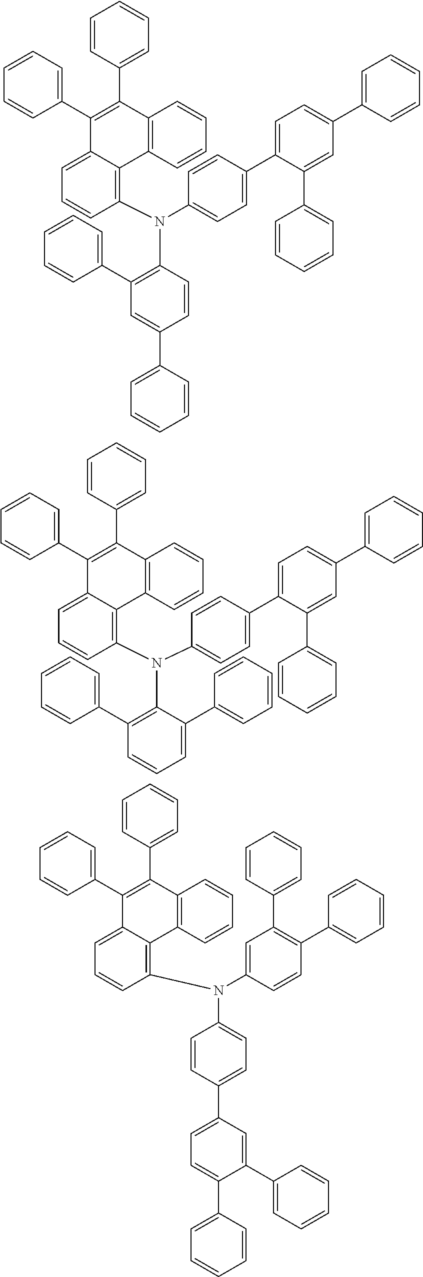

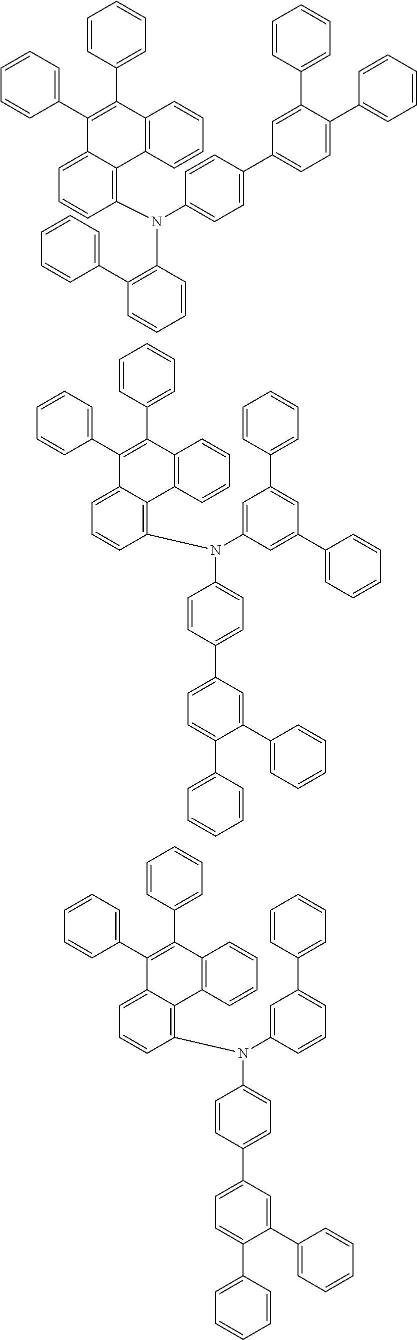

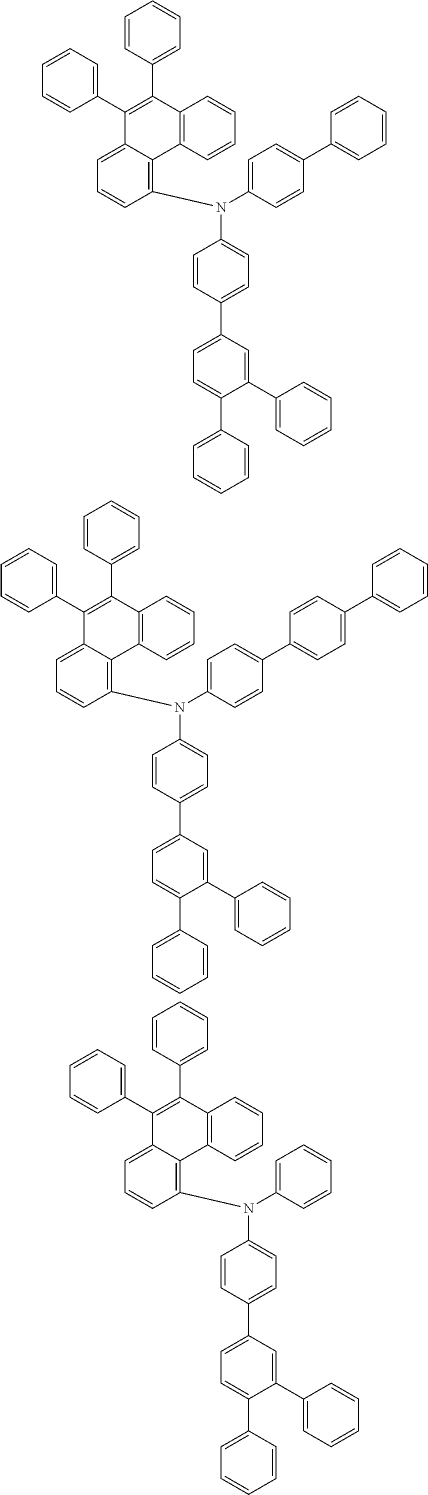

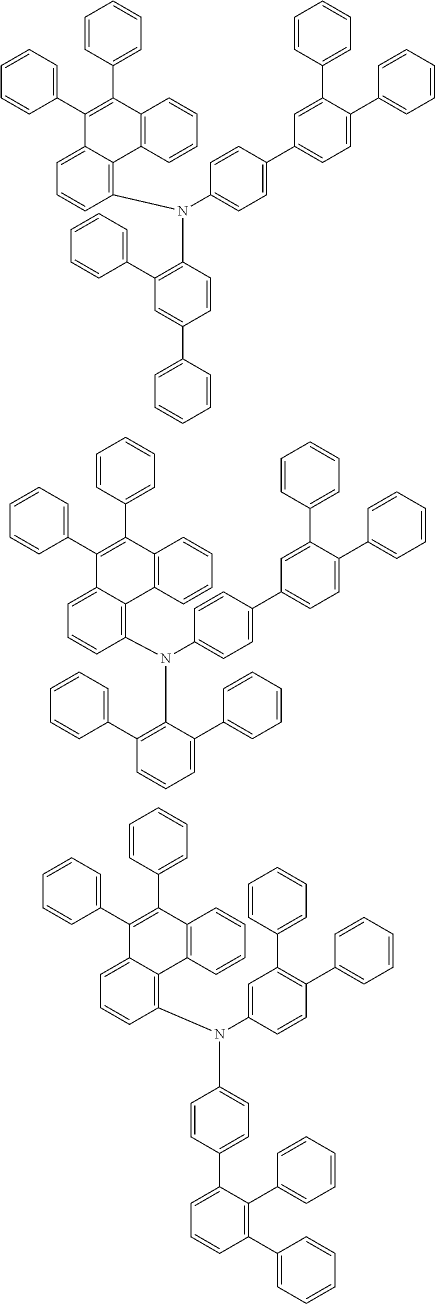

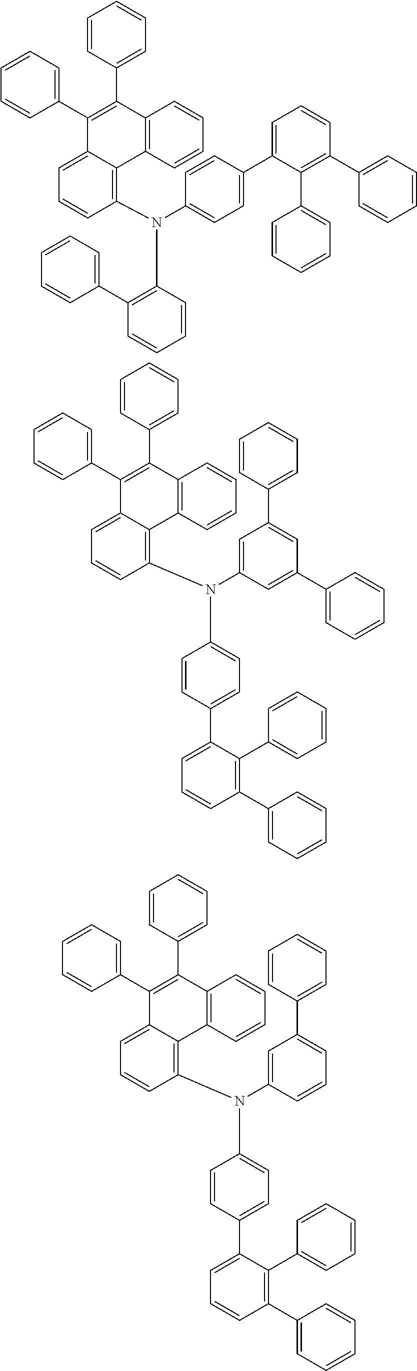

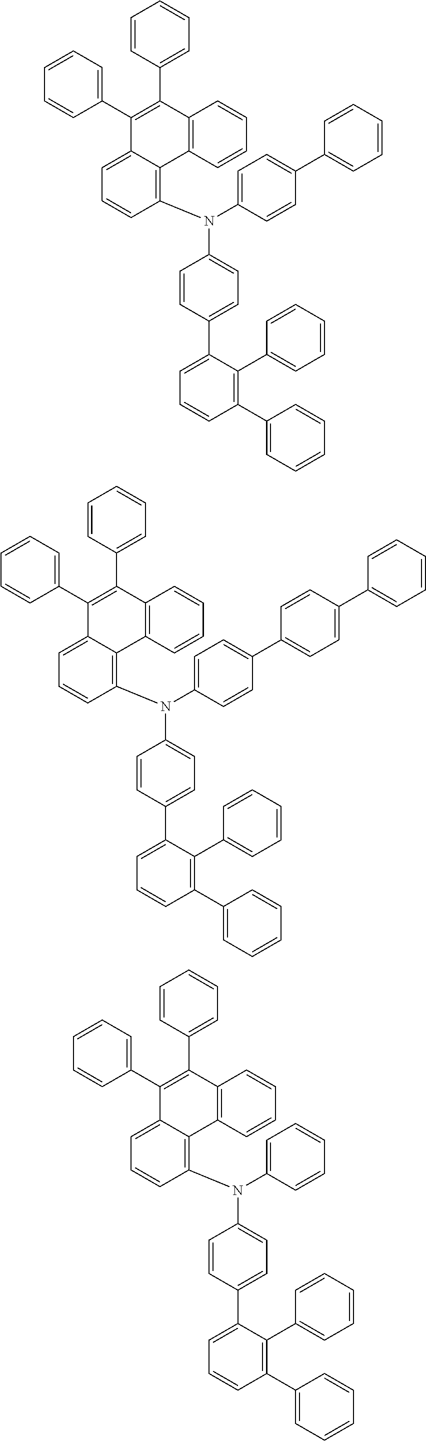

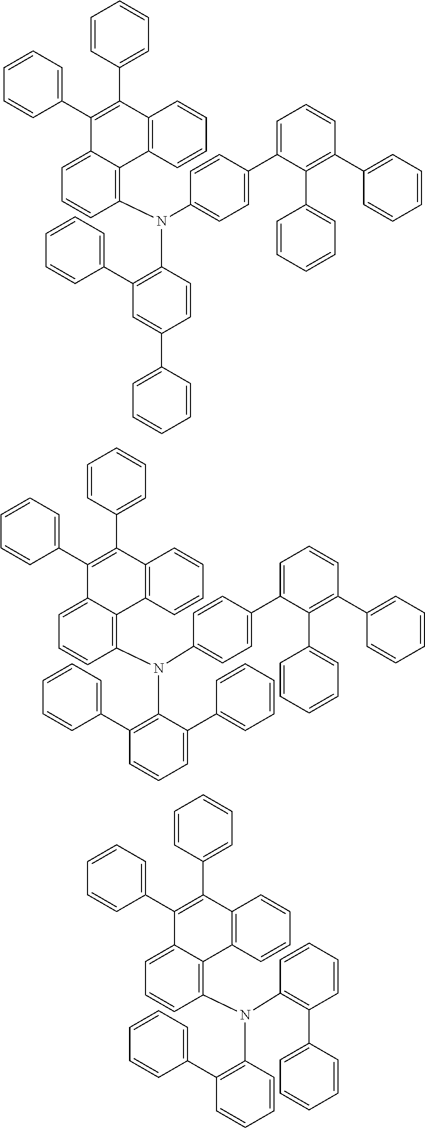

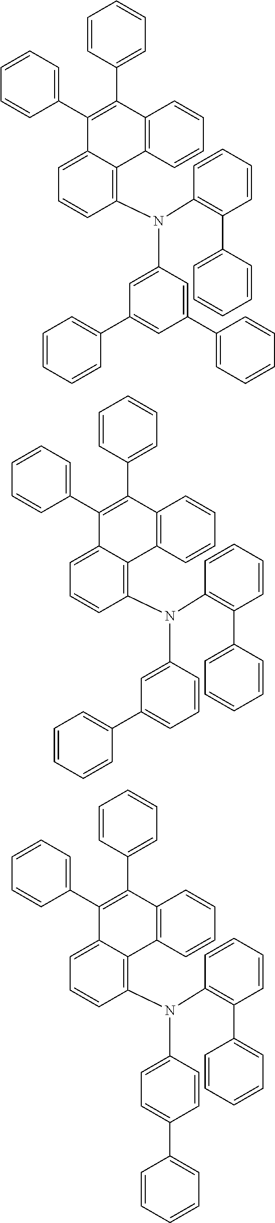

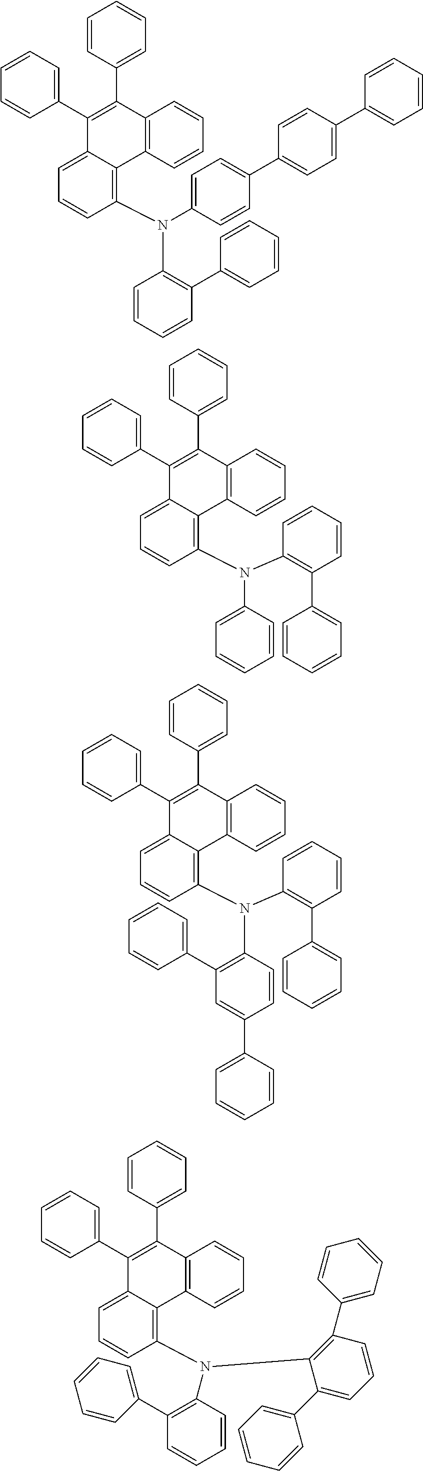

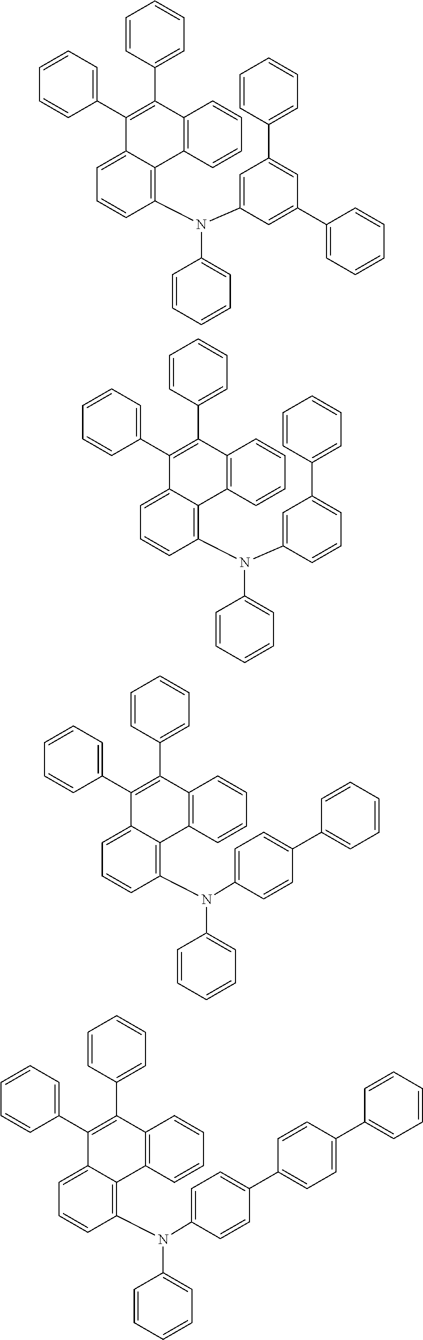

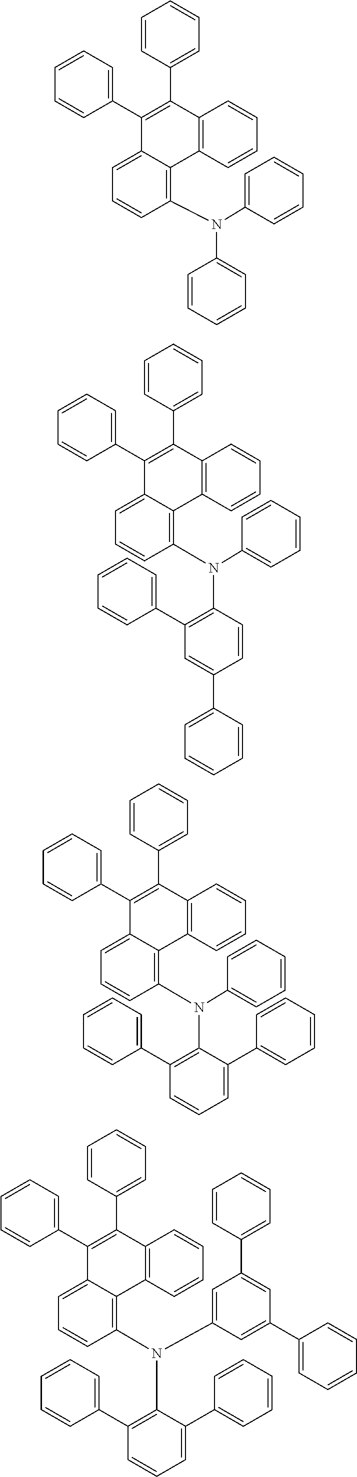

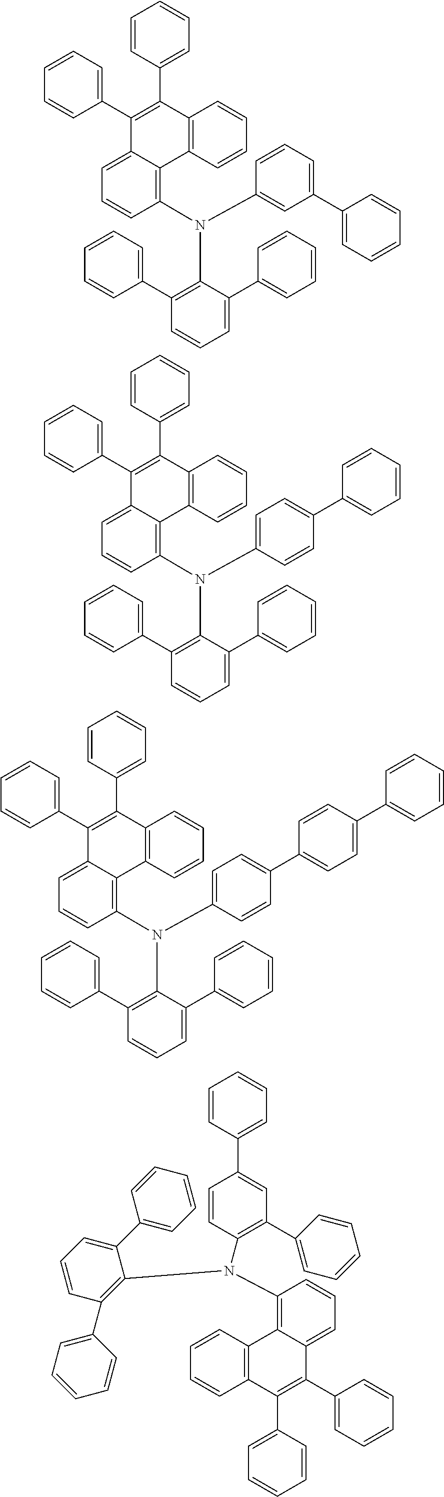

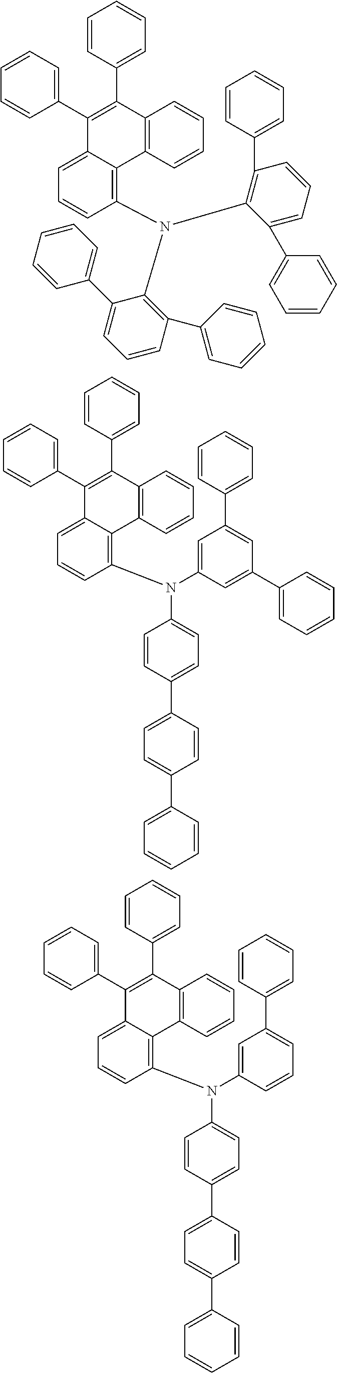

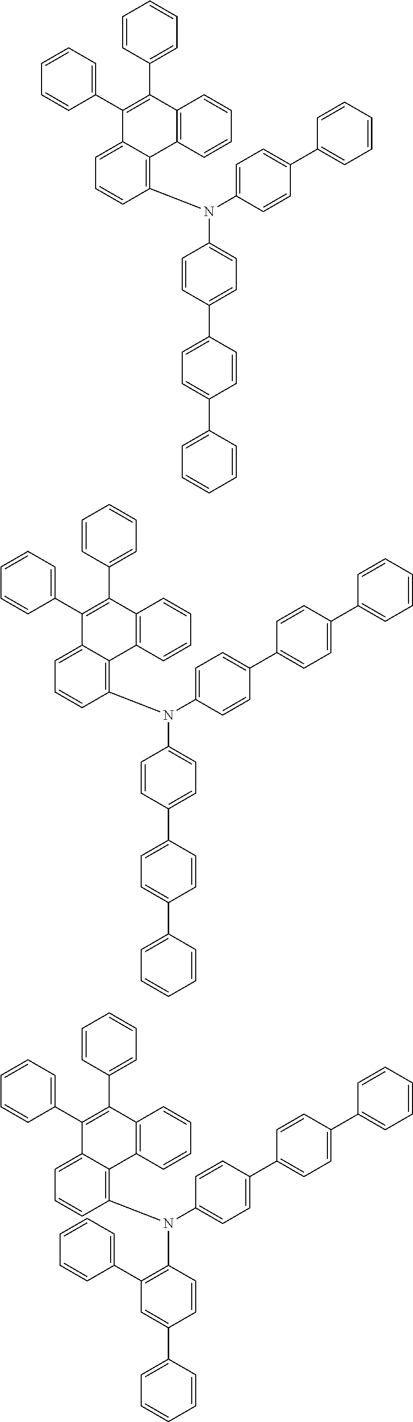 [SMILES: c1ccc(-c2ccc(-c3ccc(N(c4ccc(-c5ccccc5)cc4)c4cccc5c(-c6ccccc6)c(-c6ccccc6)c6ccccc6c45)cc3)cc2)cc1 ; c1ccc(-c2ccc(-c3ccc(N(c4ccc(-c5ccc(-c6ccccc6)cc5)cc4)c4cccc5c(-c6ccccc6)c(-c6ccccc6)c6ccccc6c45)cc3)cc2)cc1 ; c1ccc(-c2ccc(-c3ccc(N(c4ccc(-c5ccccc5)cc4-c4ccccc4)c4cccc5c(-c6ccccc6)c(-c6ccccc6)c6ccccc6c45)cc3)cc2)cc1]

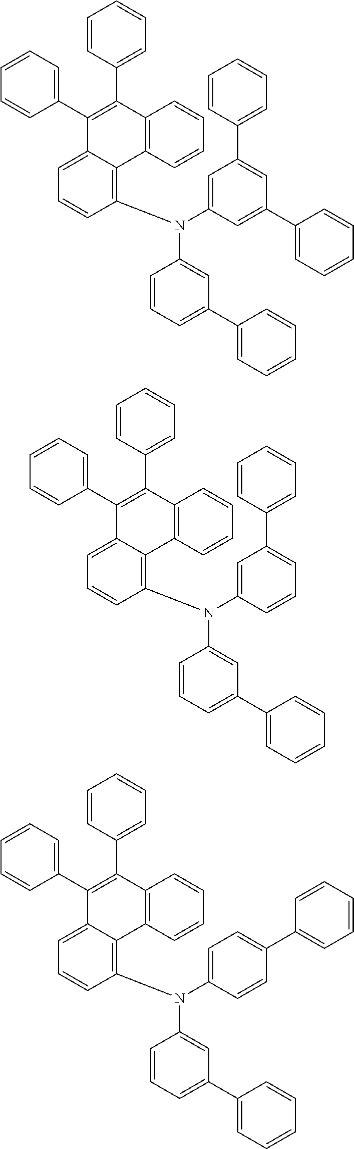 [SMILES: c1ccc(-c2cccc(N(c3cc(-c4ccccc4)cc(-c4ccccc4)c3)c3cccc4c(-c5ccccc5)c(-c5ccccc5)c5ccccc5c34)c2)cc1 ; c1ccc(-c2cccc(N(c3cccc(-c4ccccc4)c3)c3cccc4c(-c5ccccc5)c(-c5ccccc5)c5ccccc5c34)c2)cc1 ; c1ccc(-c2ccc(N(c3cccc(-c4ccccc4)c3)c3cccc4c(-c5ccccc5)c(-c5ccccc5)c5ccccc5c34)cc2)cc1]

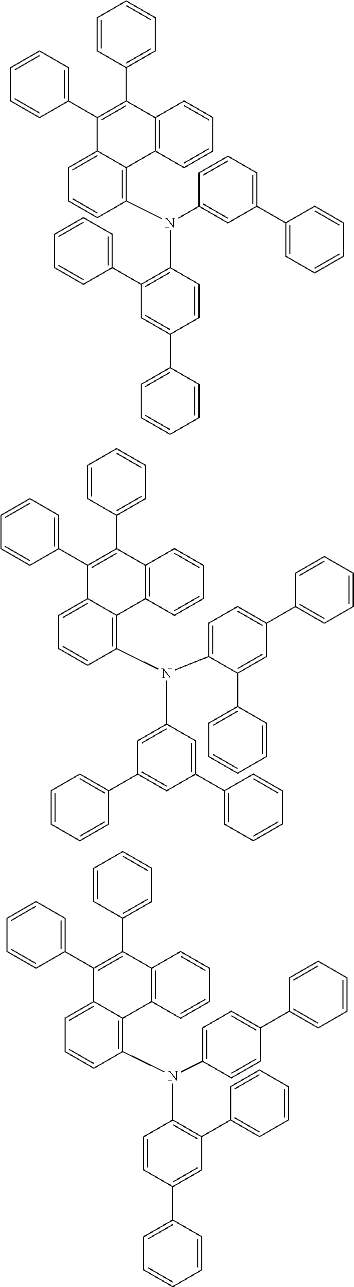

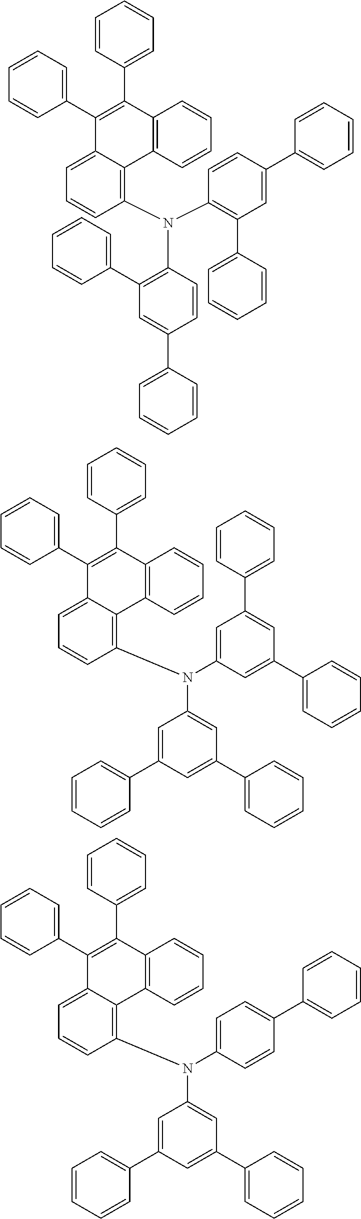

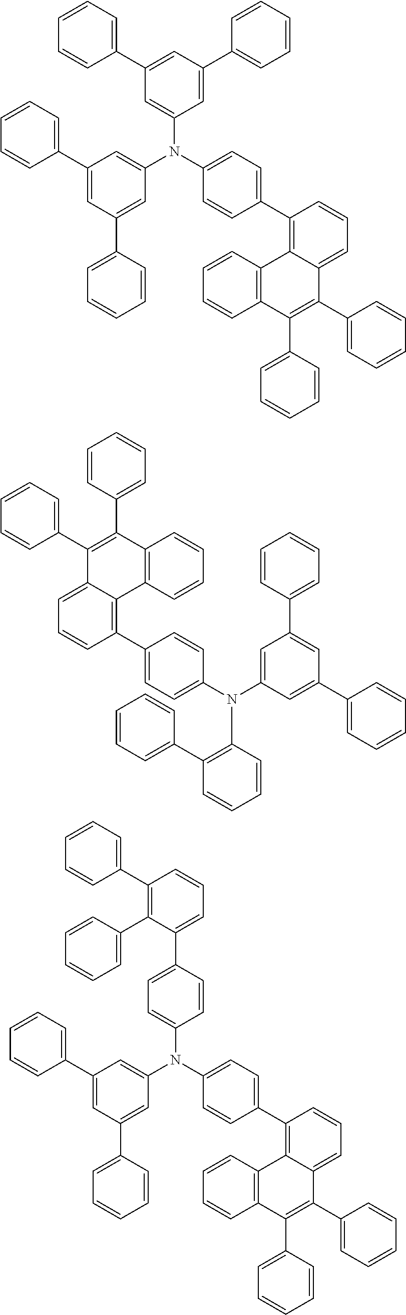 [SMILES: c1ccc(-c2cc(-c3ccccc3)cc(N(c3ccc(-c4cccc5c(-c6ccccc6)c(-c6ccccc6)c6ccccc6c45)cc3)c3cc(-c4ccccc4)cc(-c4ccccc4)c3)c2)cc1 ; c1ccc(-c2cc(-c3ccccc3)cc(N(c3ccc(-c4cccc5c(-c6ccccc6)c(-c6ccccc6)c6ccccc6c45)cc3)c3ccccc3-c3ccccc3)c2)cc1 ; c1ccc(-c2cc(-c3ccccc3)cc(N(c3ccc(-c4cccc(-c5ccccc5)c4-c4ccccc4)cc3)c3ccc(-c4cccc5c(-c6ccccc6)c(-c6ccccc6)c6ccccc6c45)cc3)c2)cc1]

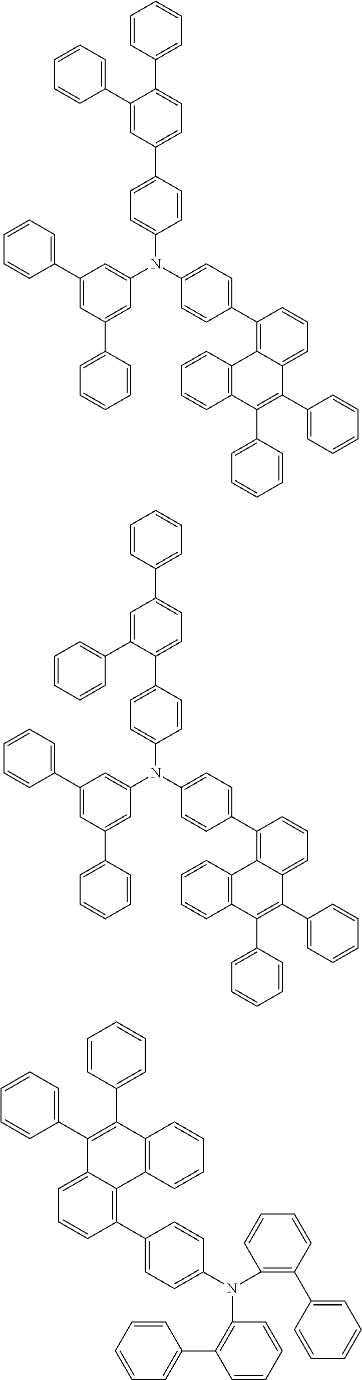 [SMILES: c1ccc(-c2cc(-c3ccccc3)cc(N(c3ccc(-c4ccc(-c5ccccc5)c(-c5ccccc5)c4)cc3)c3ccc(-c4cccc5c(-c6ccccc6)c(-c6ccccc6)c6ccccc6c45)cc3)c2)cc1 ; c1ccc(-c2cc(-c3ccccc3)cc(N(c3ccc(-c4ccc(-c5ccccc5)cc4-c4ccccc4)cc3)c3ccc(-c4cccc5c(-c6ccccc6)c(-c6ccccc6)c6ccccc6c45)cc3)c2)cc1 ; c1ccc(-c2ccccc2N(c2ccc(-c3cccc4c(-c5ccccc5)c(-c5ccccc5)c5ccccc5c34)cc2)c2ccccc2-c2ccccc2)cc1]

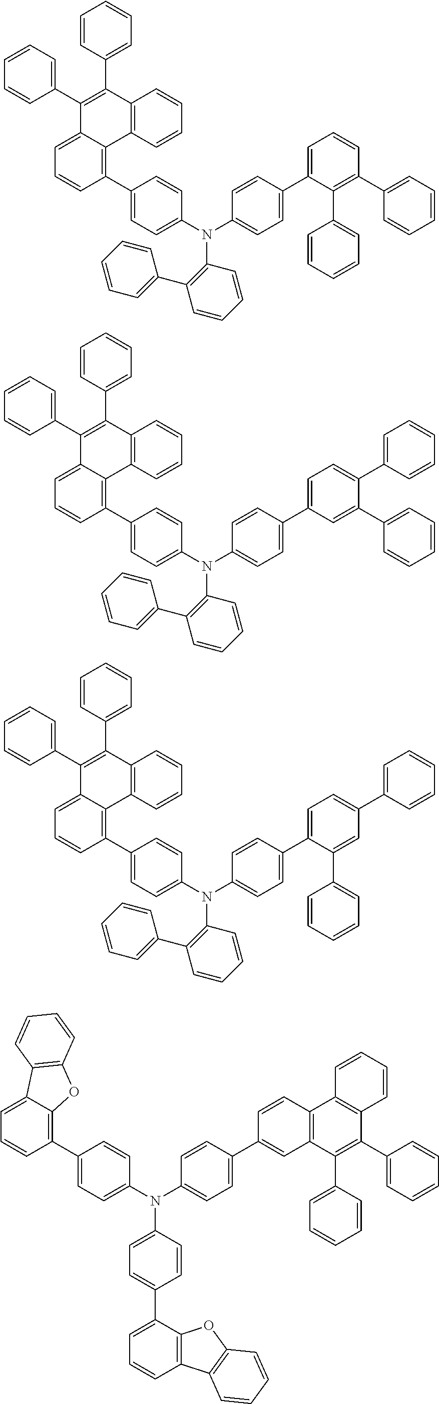 [SMILES: c1ccc(-c2ccccc2N(c2ccc(-c3cccc(-c4ccccc4)c3-c3ccccc3)cc2)c2ccc(-c3cccc4c(-c5ccccc5)c(-c5ccccc5)c5ccccc5c34)cc2)cc1 ; c1ccc(-c2ccc(-c3ccc(N(c4ccc(-c5cccc6c(-c7ccccc7)c(-c7ccccc7)c7ccccc7c56)cc4)c4ccccc4-c4ccccc4)cc3)cc2-c2ccccc2)cc1 ; c1ccc(-c2ccc(-c3ccc(N(c4ccc(-c5cccc6c(-c7ccccc7)c(-c7ccccc7)c7ccccc7c56)cc4)c4ccccc4-c4ccccc4)cc3)c(-c3ccccc3)c2)cc1 ; c1ccc(-c2c(-c3ccccc3)c3cc(-c4ccc(N(c5ccc(-c6cccc7c6oc6ccccc67)cc5)c5ccc(-c6cccc7c6oc6ccccc67)cc5)cc4)ccc3c3ccccc23)cc1]

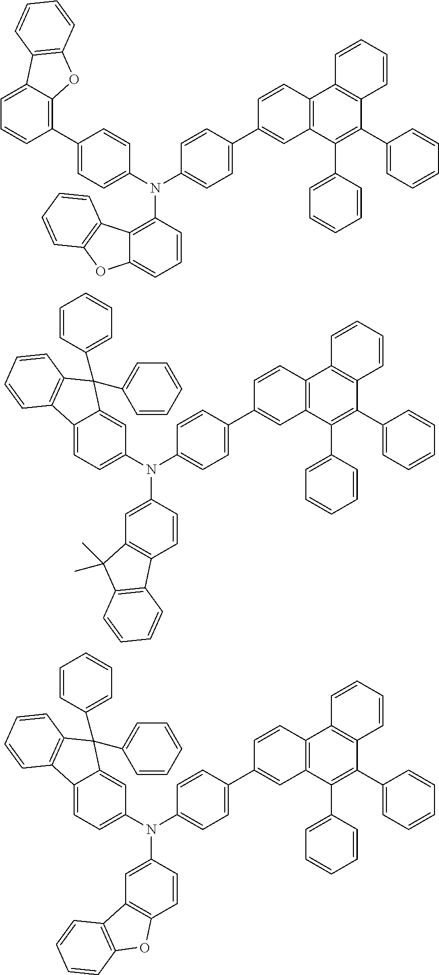

Material for Organic EL Device

The material for an organic EL device of the present invention contains the invention compound. The content of the invention compound in the material for the organic EL device is 1 mass % or more (including 100%), preferably 10 mass % or more (including 100%), more preferably 50 mass % or more (including 100%), further preferably 80 mass % or more (including 100%), particularly preferably 90 mass % or more (including 100%). The material for the organic EL device of the present invention is useful for producing the organic EL device.

Organic EL Device

In an aspect of the present invention, the organic EL device includes an anode, a cathode, and organic layers arranged between the corresponding anode and the cathode. The corresponding organic layers include a light emitting layer, and at least one layer among the corresponding organic layers contains the invention compound.

Examples of the organic layer containing the invention compound include a hole transporting zone (a hole injecting layer, a hole transporting layer, an electron blocking layer, an exciton blocking layer, etc.) provided between the anode and the light emitting layer, the light emitting layer, a space layer, and an electron transporting zone (an electron injecting layer, an electron transporting layer, a hole blocking layer, etc.) provided between the cathode and the light emitting layer, but are not limited to these. The invention compound is used preferably as a material for the hole transporting zone or the light emitting layer of a fluorescent or phosphorescent EL device, more preferably as a material for the hole transporting zone, further preferably as a material for the hole injecting layer, the hole transporting layer, the electron blocking layer, or the exciton blocking layer, particularly preferably as a material for the hole injecting layer or the hole transporting layer.

The organic EL device of the present invention may be a fluorescence or phosphorescence-emitting monochromatic light emitting device, or a fluorescence/phosphorescence-hybrid white light emitting device, and may be a simple type having a single light emitting unit or a tandem type having a plurality of light emitting units. Among these, the fluorescence emitting device is preferred. Here, the “light emitting unit” refers to a minimum unit including organic layers among which at least one layer is a light emitting layer, in which light is emitted through recombination of injected holes and electrons.

For example, as a representative device configuration of the simple-type organic EL device, the following device configuration can be exemplified.

(1) Anode/light emitting unit/cathode

Further, the light emitting unit may be a multi-layer type having a plurality of phosphorescence emitting layers or fluorescence emitting layers. In this case, a space layer may be provided between the light emitting layers for the purpose of preventing excitons generated in the phosphorescence emitting layer from diffusing into the fluorescence emitting layer. Typical layer structures of the simple-type light emitting unit are illustrated below. Layers in the brackets are optional.
(a) (hole injecting layer/)hole transporting layer/fluorescence emitting layer/electron transporting layer (/electron injecting layer)
(b) (hole injecting layer/)hole transporting layer/first fluorescence emitting layer/second fluorescence emitting layer/electron transporting layer (/electron injecting layer)
(c) (hole injecting layer/)hole transporting layer/phosphorescence emitting layer/space layer/fluorescence emitting layer/electron transporting layer (/electron injecting layer)
(d) (hole injecting layer/) hole transporting layer/first phosphorescence emitting layer/second phosphorescence emitting layer/space layer/fluorescence emitting layer/electron transporting layer (/electron injecting layer)
(e) (hole injecting layer/)hole transporting layer/phosphorescence emitting layer/space layer/first fluorescence emitting layer/second fluorescence emitting layer/electron transporting layer (/electron injecting layer)
(f) (hole injecting layer/)hole transporting layer/electron blocking layer/fluorescence emitting layer/electron transporting layer (/electron injecting layer)
(g) (hole injecting layer/)hole transporting layer/exciton blocking layer/fluorescence emitting layer/electron transporting layer (/electron injecting layer)
(h) (hole injecting layer/)first hole transporting layer/second hole transporting layer/fluorescence emitting layer/electron transporting layer (/electron injecting layer)
(i) (hole injecting layer/)first hole transporting layer/second hole transporting layer/fluorescence emitting layer/first electron transporting layer/second electron transporting layer (/electron injecting layer)
(j) (hole injecting layer/)hole transporting layer/fluorescence emitting layer/hole blocking layer/electron transporting layer (/electron injecting layer)
(k) (hole injecting layer/)hole transporting layer/fluorescence emitting layer/exciton blocking layer/electron transporting layer (/electron injecting layer)
(l) (hole injecting layer/) first hole transporting layer/second hole transporting layer/first fluorescence emitting layer/second fluorescence emitting layer/first electron transporting layer/second electron transporting layer (/electron injecting layer)
(m) (hole injecting layer/)first hole transporting layer/second hole transporting layer/third hole transporting layer/first fluorescence emitting layer/second fluorescence emitting layer/first electron transporting layer/second electron transporting layer (/electron injecting layer)

The phosphorescence and fluorescence emitting layers can exhibit different emission colors, respectively. Specifically, in the light emitting unit (f), a layer structure such as (hole injecting layer/)hole transporting layer/first phosphorescence emitting layer (red emission)/second phosphorescence emitting layer (green emission)/space layer/fluorescence emitting layer (blue emission)/electron transporting layer may be exemplified.

The electron blocking layer may be appropriately provided between each light emitting layer and the hole transporting layer or the space layer. Further, the hole blocking layer may be appropriately provided between each light emitting layer and the electron transporting layer. By providing the electron blocking layer or the hole blocking layer, it is possible to confine electrons or holes within the light emitting layer and to increase the probability of charge recombination in the light emitting layer, thereby improving the emission efficiency.

As a representative device configuration of the tandem-type organic EL device, the following device configuration can be exemplified.

(2) anode/first light emitting unit/intermediate layer/second light emitting unit/cathode

Here, for example, each of the first light emitting unit and the second light emitting unit can be independently selected from the above described light emitting units.

The intermediate layer is also generally called an intermediate electrode, an intermediate conductive layer, a charge generation layer, an electron withdrawal layer, a connection layer, or an intermediate insulating layer, and it is possible to use a known material configuration in which electrons and holes are supplied to the first light emitting unit and the second light emitting unit, respectively.

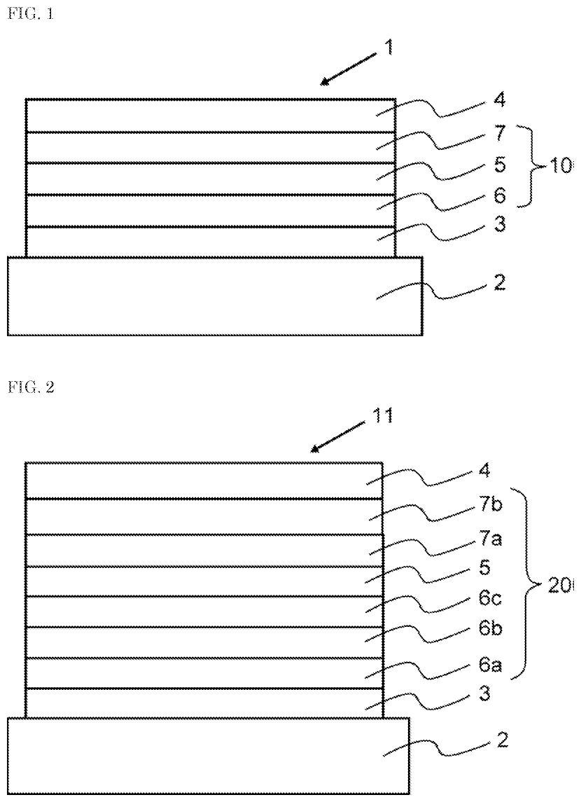

FIG. 1 is a schematic view illustrating an example of the configuration of the organic EL device of the present invention. An organic EL device 1 includes a substrate 2, an anode 3, a cathode 4, and a light emitting unit 10 arranged between the corresponding anode 3 and the cathode 4. The light emitting unit 10 has a light emitting layer 5, and has a hole transporting zone 6 (a hole injecting layer, a hole transporting layer, etc.) between the light emitting layer 5 and the anode 3, and an electron transporting zone 7 (an electron injecting layer, an electron transporting layer, etc.) between the light emitting layer 5 and the cathode 4. Further, an electron blocking layer (not illustrated), and a hole blocking layer (not illustrated) may be provided on the anode 3 side of the light emitting layer 5 and on the cathode 4 side of the light emitting layer 5, respectively. Accordingly, electrons or holes can be confined in the light emitting layer 5, so that the exciton generation efficiency in the light emitting layer 5 can be further increased.

FIG. 2 is a schematic view illustrating another configuration of the organic EL device of the present invention. An organic EL device 11 has a substrate 2, an anode 3, a cathode 4, and a light emitting unit 20 arranged between the corresponding anode 3 and the cathode 4. The light emitting unit 20 has a light emitting layer 5. A hole transporting zone arranged between the anode 3 and the light emitting layer 5 is formed by a hole injecting layer 6a, a first hole transporting layer 6b and a second hole transporting layer 6c. Further, an electron transporting zone arranged between the light emitting layer 5 and the cathode 4 is formed by a first electron transporting layer 7a and a second electron transporting layer 7b.

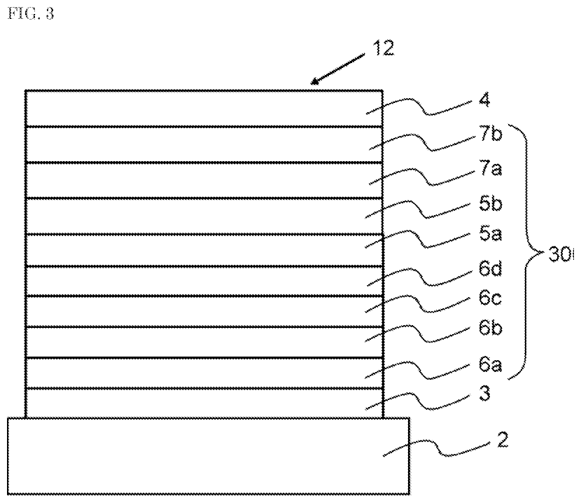

FIG. 3 is a schematic view illustrating another configuration of the organic EL device of the present invention. An organic EL device 12 has a substrate 2, an anode 3, a cathode 4, and a light emitting unit 30 arranged between the corresponding anode 3 and the cathode 4. The light emitting unit 30 has a first light emitting layer 5a and a second light emitting layer 5b. A hole transporting zone arranged between the anode 3 and the first light emitting layer 5a is formed by a hole injecting layer 6a, a first hole transporting layer 6b, a second hole transporting layer 6c, and a third hole transporting layer 6d. Further, an electron transporting zone arranged between the second light emitting layer 5b and the cathode 4 is formed by a first electron transporting layer 7a and a second electron transporting layer 7b.

In the present invention, a host combined with a fluorescent dopant material (a fluorescence emitting material) is called a fluorescent host, and a host combined with a phosphorescent dopant material is called a phosphorescent host. The fluorescent host and the phosphorescent host are not distinguished from each other merely by molecular structures. That is, the phosphorescent host means a material that forms a phosphorescence emitting layer containing a phosphorescent dopant, but does not mean that the use as a material for forming a fluorescence emitting layer is not possible. The same also applies to the fluorescent host.

Substrate

The substrate is used as a support for the organic EL device. As for the substrate, for example, a plate of glass, quartz, plastic or the like can be used. Further, a flexible substrate may be used. Examples of the flexible substrate include plastic substrates made of polycarbonate, polyarylate, polyethersulfone, polypropylene, polyester, polyvinyl fluoride, and polyvinyl chloride. Further, an inorganic deposition film can also be used.

Anode

For the anode to be formed on the substrate, it is desirable to use a metal, an alloy, an electrically conductive compound, a mixture thereof or the like with a large work function (specifically 4.0 eV or more). Specifically, examples thereof include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chromium (Cr), molybdenum (Mo), iron (Fe), cobalt (Co), copper (Cu), palladium (Pd), titanium (Ti), nitrides of the metals (for example, titanium nitride) or the like may be exemplified.

These materials are usually deposited by a sputtering method. For example, indium oxide-zinc oxide, and indium oxide containing tungsten oxide and zinc oxide can be formed through a sputtering method by using a target obtained by adding 1 to 10 wt % of zinc oxide to indium oxide, and a target containing 0.5 to 5 wt % of tungsten oxide and 0.1 to 1 wt % of zinc oxide relative to indium oxide, respectively. In addition, a vacuum deposition method, a coating method, an inkjet method, a spin coating method or the like may be used for the production.

Hole Transporting Zone

As described above, the organic layers include the hole transporting zone between the anode and the light emitting layer. The hole transporting zone is constituted by the hole injecting layer, the hole transporting layer, the electron blocking layer, etc. It is desirable that the hole transporting zone contains the invention compound. It is preferable that among these layers constituting the hole transporting zone, at least one layer contains the invention compound, and in particular, it is more preferable that the hole transporting layer contains the invention compound.

The hole injecting layer formed in contact with the anode is formed by using a material that facilitates hole injection regardless of the work function of the anode. Thus, it is possible to use materials generally used as electrode materials (e.g., metals, alloys, electrically conductive compounds, and mixtures thereof, and elements belonging to Group 1 or Group 2 in the periodic table of elements).

It is also possible to use materials with a small work function, e.g., elements belonging to Group 1 or Group 2 in the periodic table of elements, i.e., alkali metals such as lithium (Li) or cesium (Cs), alkaline earth metals such as magnesium (Mg), calcium (Ca), and strontium (Sr), and alloys containing these (e.g., MgAg, AlLi), and rare earth metals such as europium (Eu), and ytterbium (Yb) and alloys containing these. When the anode is formed by using an alkali metal, an alkaline earth metal, or an alloy containing these, a vacuum deposition method or a sputtering method can be used. Further, when silver paste or the like is used, a coating method, an inkjet method or the like can be used.

Hole Injecting Layer

The hole injecting layer is a layer containing a material with a high hole-injection capability (a hole injecting material), and is formed between the anode and the light emitting layer, or between the anode and the hole transporting layer if the hole transporting layer exists.

As for the hole injecting material other than the invention compound, it is possible to use molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, etc.

Examples of the hole injecting layer material include aromatic amine compounds, which are low molecular organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-[4-[N′-(3-methylphenyl)-N′-phenylamino]phenyl}-N-phenylaminoThiphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

High molecular compounds (oligomers, dendrimers, polymers, etc.) can also be used. Examples thereof include high molecular compounds such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N, N′-bis(4-butylphenyl)-N,N′-bis(phenyl)benzidine] (abbreviation: Poly-TPD). Further, high molecular compounds to which acids are added, such as poly(3,4-ethylenedioxythiophene)/poly(styrenesulfonic acid) (PEDOT/PSS), and polyaniline/poly(styrenesulfonic acid) (PAni/PSS), can also be used.

Further, it is also desirable to use an acceptor material represented by the following formula (K), such as a hexaazatriphenylene (HAT) compound.

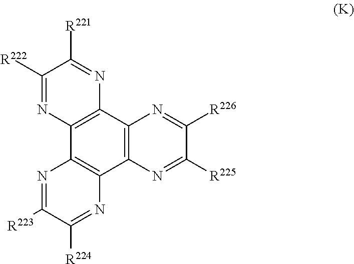

(In the formula, each of R²²¹ to R²²⁶ independently represents a cyano group, —CONH₂, a carboxy group, or —COOR²²⁷ (R²²⁷ represents an alkyl group having 1 to 20 carbon atoms or a cycloalkyl group having 3 to 20 carbon atoms). Further, adjacent two selected from R²²¹ and R²²², R²²³ and R²²⁴, and R²²⁵ and R²²⁶ may be bonded to each other to form a group represented by —CO—O—CO—.)

Examples of R²²⁷ include a methyl group, an ethyl group, a n-propyl group, an isopropyl group, a n-butyl group, an isobutyl group, a t-butyl group, a cyclopentyl group, and a cyclohexyl group.

Hole Transporting Layer

The hole transporting layer is a layer containing a material with a high hole transporting capability (a hole transporting material), and is formed between the anode and the light emitting layer, or between the light emitting layer and the hole injecting layer if the hole injecting layer exists. The invention compound may be used alone or in combination with the following compounds, in the hole transporting layer.

The hole transporting layer may have a single-layer structure or a multi-layer structure including two or more layers. For example, the hole transporting layer may have a two-layer structure including a first hole transporting layer (anode side) and a second hole transporting layer (cathode side). That is, the hole transporting zone may include the first hole transporting layer on the anode side and the second hole transporting layer on the cathode side. Further, the hole transporting layer may have a three-layer structure including a first hole transporting layer, a second hole transporting layer, and a third hole transporting layer in order from the anode side. That is, the third hole transporting layer may be arranged between the second hole transporting layer and the light emitting layer.

In an aspect of the present invention, the hole transporting layer with the single-layer structure is preferably adjacent to the light emitting layer, and the hole transporting layer closest to the cathode in the multi-layer structure, for example, the second hole transporting layer in the two-layer structure or the third hole transporting layer in the three-layer structure, is preferably adjacent to the light emitting layer. In another aspect of the present invention, the electron blocking layer to be described below may be interposed between the hole transporting layer with the single-layer structure and the light emitting layer, or between the light emitting layer and the hole transporting layer closest to the light emitting layer in the multi-layer structure.

When the hole transporting layer has the two-layer structure, at least one of the first hole transporting layer and the second hole transporting layer contains the invention compound. That is, the invention compound may be contained in either the first hole transporting layer or the second hole transporting layer, or may be contained in both. In an aspect of the present invention, the invention compound is preferably contained in the second hole transporting layer. That is, it is desirable that the invention compound is contained in only the second hole transporting layer, or the invention compound is contained in the first hole transporting layer and the second hole transporting layer.

When the hole transporting layer has the three-layer structure, at least one of the first to third hole transporting layers contains the invention compound. That is, the invention compound may be contained in only one of the first to third hole transporting layers, may be contained in only any two, or may be contained in all of them. In an aspect of the present invention, the invention compound is preferably contained in the third hole transporting layer. That is, it is desirable that the invention compound is contained in only the third hole transporting layer, or the invention compound is contained in the third hole transporting layer and either or both of the first hole transporting layer and the second hole transporting layer.

In an aspect of the present invention, the above invention compound contained in each hole transporting layer is preferably a protium compound from the viewpoint of production cost. The protium compound refers to the invention compound in which all hydrogen atoms in the invention compound are protium atoms.

Therefore, the present invention includes an organic EL device, in which one or both of the first hole transporting layer and the second hole transporting layer (in the case of the two-layer structure) or at least one of the first to third hole transporting layers contains the invention compound substantially composed of only the protium compound. “The invention compound substantially composed of only the protium compound” means that the content of the protium compound in the total amount of the invention compound is 90 mol % or more, preferably 95 mol % or more, more preferably 99 mol % or more (each including 100%).

Other than the invention compound, for example, an aromatic amine compound, a carbazole derivative, and an anthracene derivative can be used as for the hole transporting layer material.

Examples of the aromatic amine compound include 4,4′-bis[N-(1-naphthyl)-N-phenylamino]biphenyl (abbreviation: NPB) or N,N′-bhis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The compound has a hole mobility of 10⁻⁶ cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth). High molecular compounds such as poly(N-vinylcarbazole) (abbreviation: PVK) or poly(4-vinyltriphenylamine) (abbreviation: PVTPA) can also be used.

However, a compound other than the above may be used as long as the compound has a hole transporting capability higher than an electron transporting capability.

In the organic EL device having the hole transporting layer with the two-layer structure in the present invention, the first hole transporting layer preferably contains one or more types of compounds represented by the following formula (11) or formula (12).

In the organic EL device having the hole transporting layer with the three-layer structure in the present invention, one or both of the first hole transporting layer and the second hole transporting layer preferably contains one or more types of compounds represented by the following formula (11) or (12).

In the organic EL device having the hole transporting layer with a n-layer structure (n is an integer of 4 or more) in the present invention, at least one layer of the first hole transporting layer to the (n−1)th hole transporting layer preferably contains one or more types of compounds represented by the following formula (11) or formula (12).

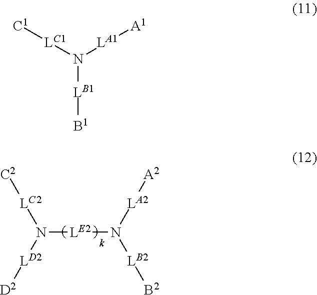

[In the formula (11) and formula (12),
each of L^{A1}, L^{B1}, L^{C1}, L^{A2}, L^{B2}, L^{C2} and L^{D2} is independently a single bond, a substituted or unsubstituted arylene group having 6 to 50 ring carbon atoms, or a substituted or unsubstituted divalent heterocyclic group having 5 to 50 ring atoms,
k is 1, 2, 3 or 4,
when k is 1, L^{E2} is a substituted or unsubstituted arylene group having 6 to 50 ring carbon atoms, or a substituted or unsubstituted divalent heterocyclic group having 5 to 50 ring atoms,
when k is 2, 3 or 4, two, three or four L^{E2}'s are the same or different,
when k is 2, 3 or 4, L^{E2}'s are bonded to each other to form a substituted or unsubstituted monocyclic ring, are bonded to each other to form a substituted or unsubstituted condensed ring, or are not bonded to each other,
L^{E2} that does not form the monocyclic ring, and does not form the condensed ring is a substituted or unsubstituted arylene group having 6 to 50 ring carbon atoms, or a substituted or unsubstituted divalent heterocyclic group having 5 to 50 ring atoms,
each of A¹, B¹, C¹, A², B², C², and D² is independently a substituted or unsubstituted aryl group having 6 to 50 ring carbon atoms, a substituted or unsubstituted heterocyclic group having 5 to 50 ring atoms, or —Si(R′₉₀₁)(R′₉₀₂)(R′₉₀₃),
each of R′₉₀₁, R′₉₀₂ and R′₉₀₃ is independently a substituted or unsubstituted aryl group having 6 to 50 ring carbon atoms,
when there are R′₉₀₁'s, R′₉₀₁'s are the same or different, when there are R′₉₀₂'s, E′₉₀₂'s are the same or different, and when there are R′₉₀₃s', R′₉₀₃'s are the same or different.]

In the formula (11) and the formula (12), it is preferable that each of A¹, B¹, C¹, A², B², C², and D² is independently selected from a substituted or unsubstituted phenylene group, a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, and a substituted or unsubstituted carbazolyl group.

Further, more preferably, at least one of A₁, B¹ and C¹ in the formula (11), and at least one of A², B², C² and D² in the formula (12), are each a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted carbazolyl group.

The fluorenyl group that A¹, B¹, C¹, A², B², C², and D² can take may have a substituent at the 9-position, and may be, for example, a 9,9-dimethylfluorenyl group, or a 9,9-diphenylfluorenyl group. Further, substituents at the 9-position may form a ring, and, for example, substituents at the 9-position may form a fluorene skeleton or a xanthene skeleton.

Each of L^{A1}, L^{B1}, L^{C1}, L^{A2}, L^{B2}, L^{C2} and L^{D2} is preferably independently a single bond, or a substituted or unsubstituted arylene group having 6 to 12 ring carbon atoms.

As specific examples of the compounds represented by the formula (11) and the formula (12), for example, the following compounds may be exemplified.

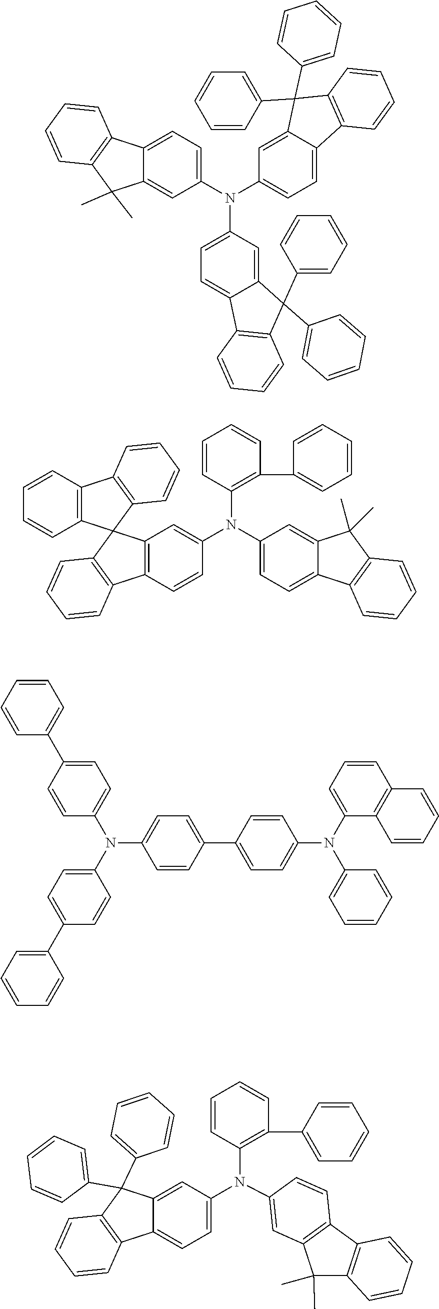

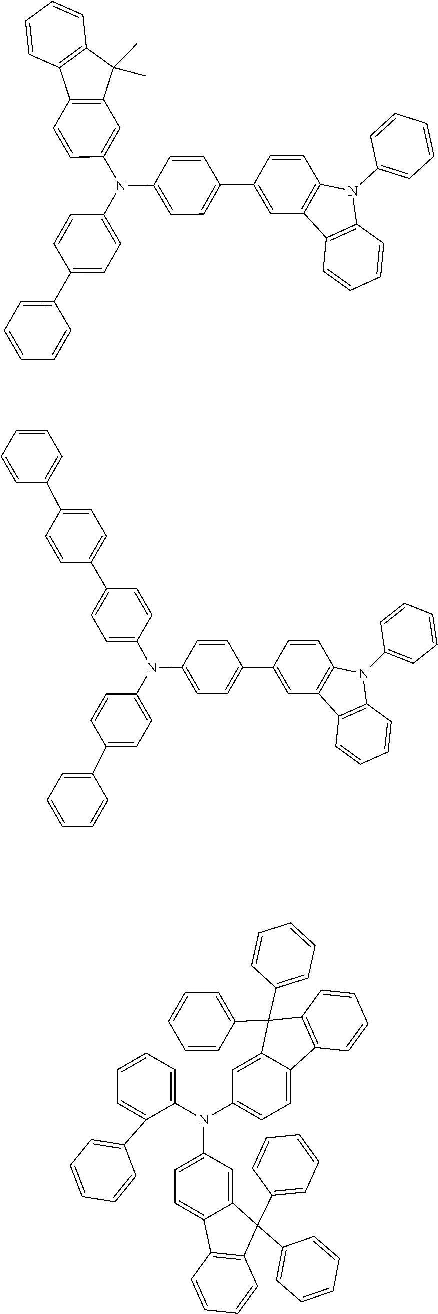 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

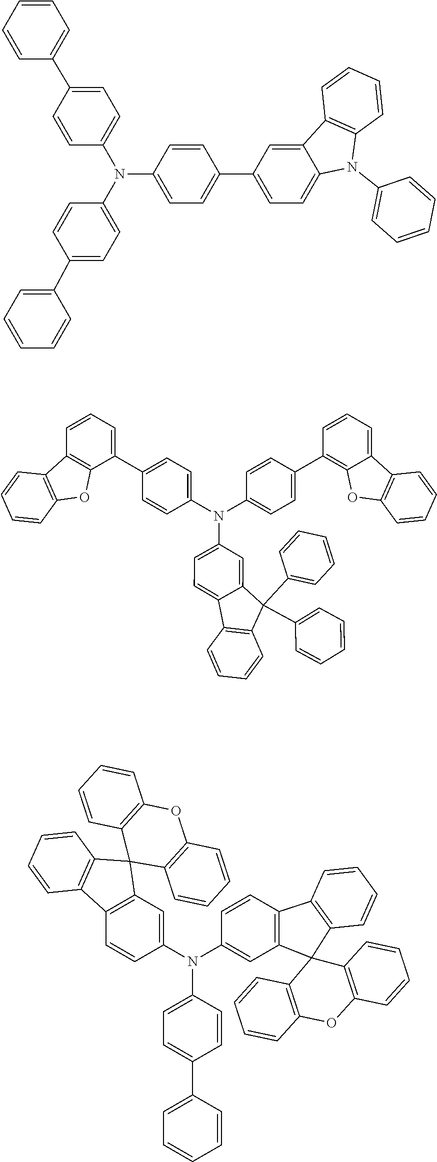 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1]

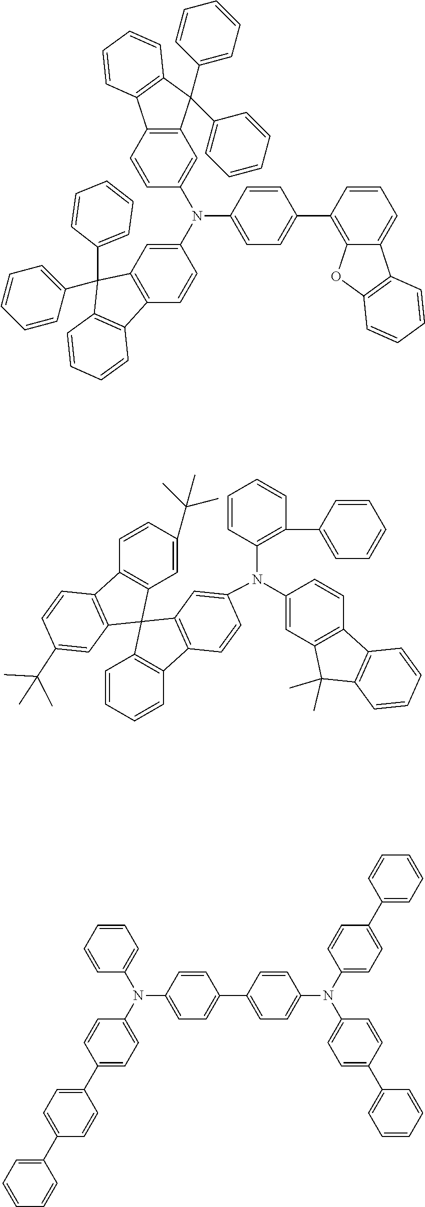 [SMILES: c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

Dopant Material of Light Emitting Layer

The light emitting layer is a layer containing a material having a high light emitting capability (a dopant material), and various materials can be used. For example, a fluorescence emitting material or a phosphorescence emitting material can be used as the dopant material. The fluorescence emitting material is a compound that emits light from a singlet excited state, and the phosphorescence emitting material is a compound that emits light from a triplet excited state.

In an aspect of the organic EL device according to the present invention, the light emitting layer is a single layer.

Further, in another aspect of the organic EL device according to the present invention, the light emitting layer includes a first light emitting layer and a second light emitting layer.

As for a blue-based fluorescence emitting material that can be used for the light emitting layer, a pyrene derivative, a styrylamine derivative, a chrysene derivative, a fluoranthene derivative, a fluorene derivative, a diamine derivative, a triarylamine derivative or the like can be used. Specific examples thereof include N,N′-bis[4-(9H-carbazole 9-yl)phenyl]-N, N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

As for a green-based fluorescence emitting material that can be used for the light emitting layer, an aromatic amine derivative or the like can be used. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N, N′,N′-triphenyl-1,4-phenylenediamine (abbreviation: 2DPAPA), N-[9,10-bis(1,1′-biphenyl-2-yl)-2-anthryl]-N, N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

As for a red-based fluorescence emitting material that can be used for the light emitting layer, a tetracene derivative, a diamine derivative or the like can be used. Specific examples thereof include N, N, N′,N′-tetrakis(4-methylphenyl)tetracene-5,11-diamine (abbreviation: p-mPhTD), and 7,14-diphenyl-N, N, N′,N′-tetrakis(4-methylphenyl)acenaphtho[1,2-a]fluoranthene-3,10-diamine (abbreviation: p-mPhAFD).

In an aspect of the present invention, it is desirable that the light emitting layer contains the fluorescence emitting material (fluorescent dopant material).

As for a blue-based phosphorescence emitting material that can be used for the light emitting layer, a metal complex such as an iridium complex, an osmium complex, or a platinum complex is used. Specific examples thereof include bis[2-(4′,6′-difluorophenyl)pyridinate-N, C2′]iridium(III) tetrakis(1-pyrazolyl)borate (abbreviation: FIr6), bis[2-(4′,6′-difluorophenyl)pyridinate-N, C2′]iridium(III) picolinate (abbreviation: FIrpic), bis[243′,5′bistrifluoromethylphenyl)pyridinate-N,C2′]iridium(III) picolinate (abbreviation: Ir(CF3ppy)2(pic)), and bis[2-(4′,6′-difluorophenyl)pyridinate-N,C2′]iridium(III) acetylacetonate (abbreviation: Flracac).

As for a green-based phosphorescence emitting material that can be used for the light emitting layer, an iridium complex or the like is used. Examples thereof include tris(2-phenylpyridinate-N, C2′)iridium(III) (abbreviation: Ir(ppy)3), bis(2-phenylpyridinate-N, C2′) iridium(III) acetylacetonate (abbreviation: Ir(ppy)2(acac)), bis(1,2-diphenyl-1H-benzoimidazolato)iridium(III) acetylacetonate (abbreviation: Ir(pbi)2(acac)), and bis(benzo[h]quinolinato)iridium(III) acetylacetonate (abbreviation: Ir(bzq)2(acac)).

As for a red-based phosphorescence emitting material that can be used for the light emitting layer, a metal complex such as an iridium complex, a platinum complex, a terbium complex, or a europium complex is used. Specific examples thereof include organic metal complexes such as bis[2-(2′-benzo[4,5-α]thienyl)pyridinato-N, C3′]iridium(III) acetylacetonate (abbreviation: Ir(btp)2(acac)), bis(1-phenylisoquinolinato-N, C2′)iridium(III) acetylacetonate (abbreviation: Ir(piq)2(acac)), (acetylacetonate)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrinplatinum(II) (abbreviation: PtOEP).

Further, rare earth metal complexes such as tris(acetylacetonate)(monophenanthroline)terbium(III) (abbreviation: Tb(acac)₃(Phen)), tris(1,3-diphenyl-1,3-propanedionato)(monophenanthroline)europium(III) (abbreviation: Eu(DBM)3(Phen)), and tris[1-(2-thenoyl)-3,3,3-trifluoroacetonato](monophenanthroline)europium(III) (abbreviation: Eu(TTA)3(Phen)) emit light from rare earth metal ions (electron transition between different multiplicities), and thus may be used as the phosphorescence emitting material.

Host Material of Light Emitting Layer

The light emitting layer may have a configuration in which the above described dopant material is dispersed in another material (a host material). It is desirable to use a material that has a higher lowest unoccupied orbital level (LUMO level) and a lower highest occupied orbital level (HOMO level) than the dopant material.

As for the host material, for example,
(1) a metal complex such as an aluminum complex, a beryllium complex, or a zinc complex,
(2) a heterocyclic compound such as an oxadiazole derivative, a benzoimidazole derivative, or a phenanthroline derivative,
(3) a fused aromatic compound such as a carbazole derivative, an anthracene derivative, a phenanthrene derivative, a pyrene derivative, or a chrysene derivative, or
(4) an aromatic amine compound such as a triarylamine derivative or a fused polycyclic aromatic amine derivative is used.

For example, it is possible to use metal complexes such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato) (4-phenylphenolato)aluminum(III) (abbreviation: BAlq), bis(8-quinolinolato)zinc(II) (abbreviation: Znq), bis[2-(2-benzooxazolyl)phenolato]zinc(II) (abbreviation: ZnPBO), and bis[2-(2-benzothiazolyl)phenolato]zinc(II) (abbreviation: ZnBTZ);

heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzoimidazole) (abbreviation: TPBI), bathophenanthroline (abbreviation: BPhen), and bathocuproine (abbreviation: BCP);

fused aromatic compounds such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′(stilbene-3,3′-diyl)diphenanthrene (abbreviation: DPNS), 9,9′(stilbene-4,4′-diyOdiphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and aromatic amine compounds such as N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N, 9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N, 9-diphenyl-9H-carbazole-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N, N′-diphenyl-[1,1′-biphenyl]-4,4′-diamine (abbreviation: TPD), 4,4′-bis[N-(9,9-dimethylfluorene-2-yl)-N-phenylamino]biphenyl (abbreviation: DFLDPBi), and 4,4′-bis[N-(spiro-9,9′-bifluorene-2-yl)-N-phenylamino]biphenyl (abbreviation: BSPB). A plurality of types of host materials may be used.

In particular, in the case of a blue fluorescent device, it is desirable to use the following anthracene compounds as the host material.

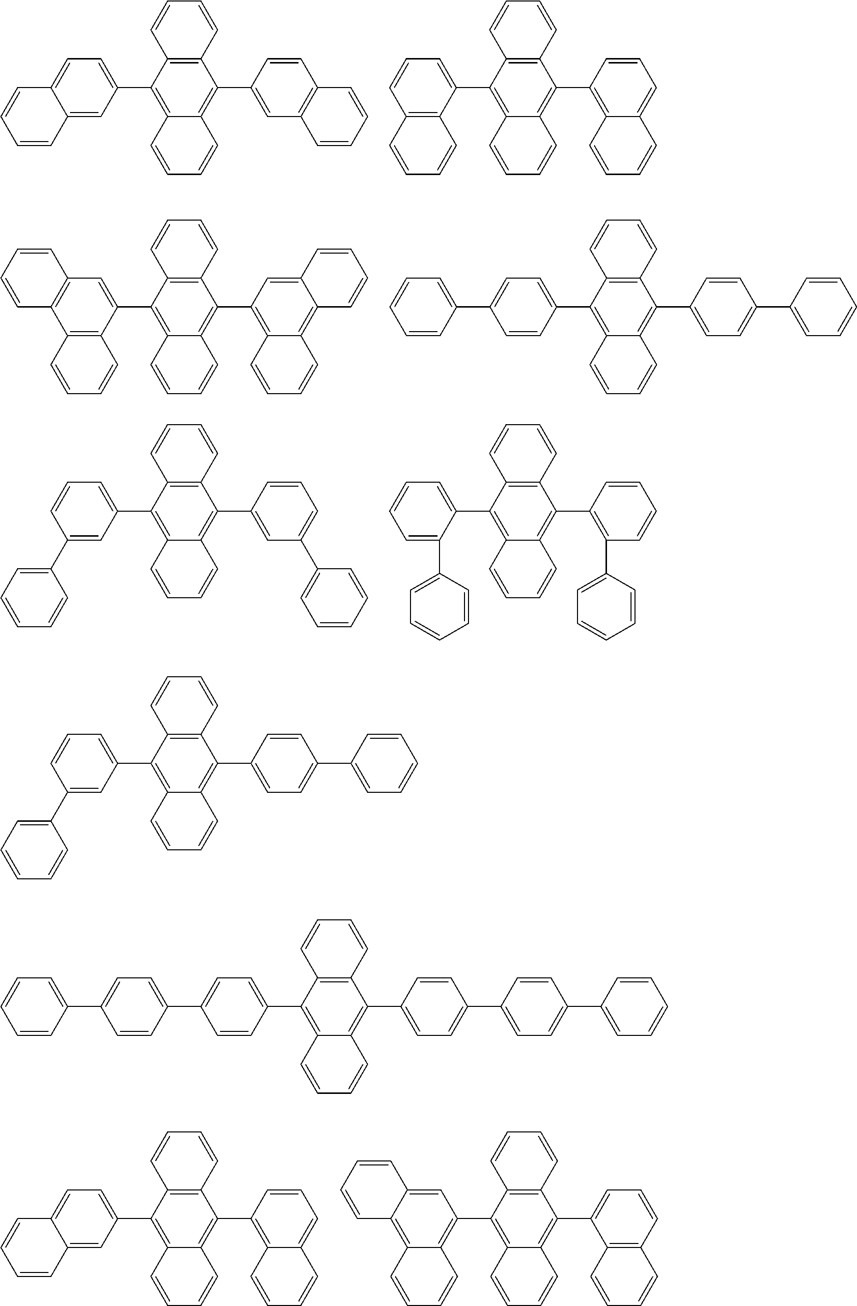

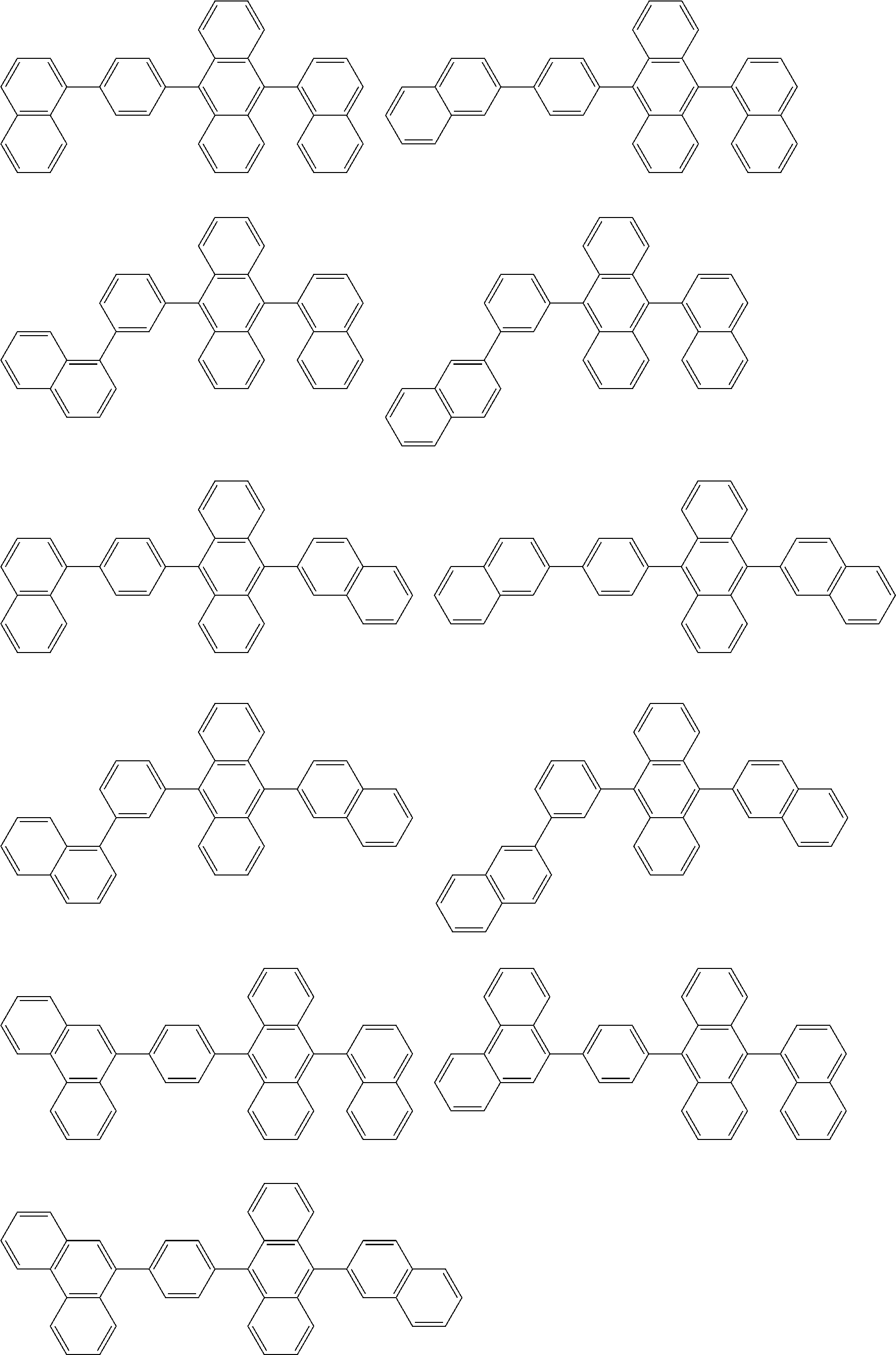

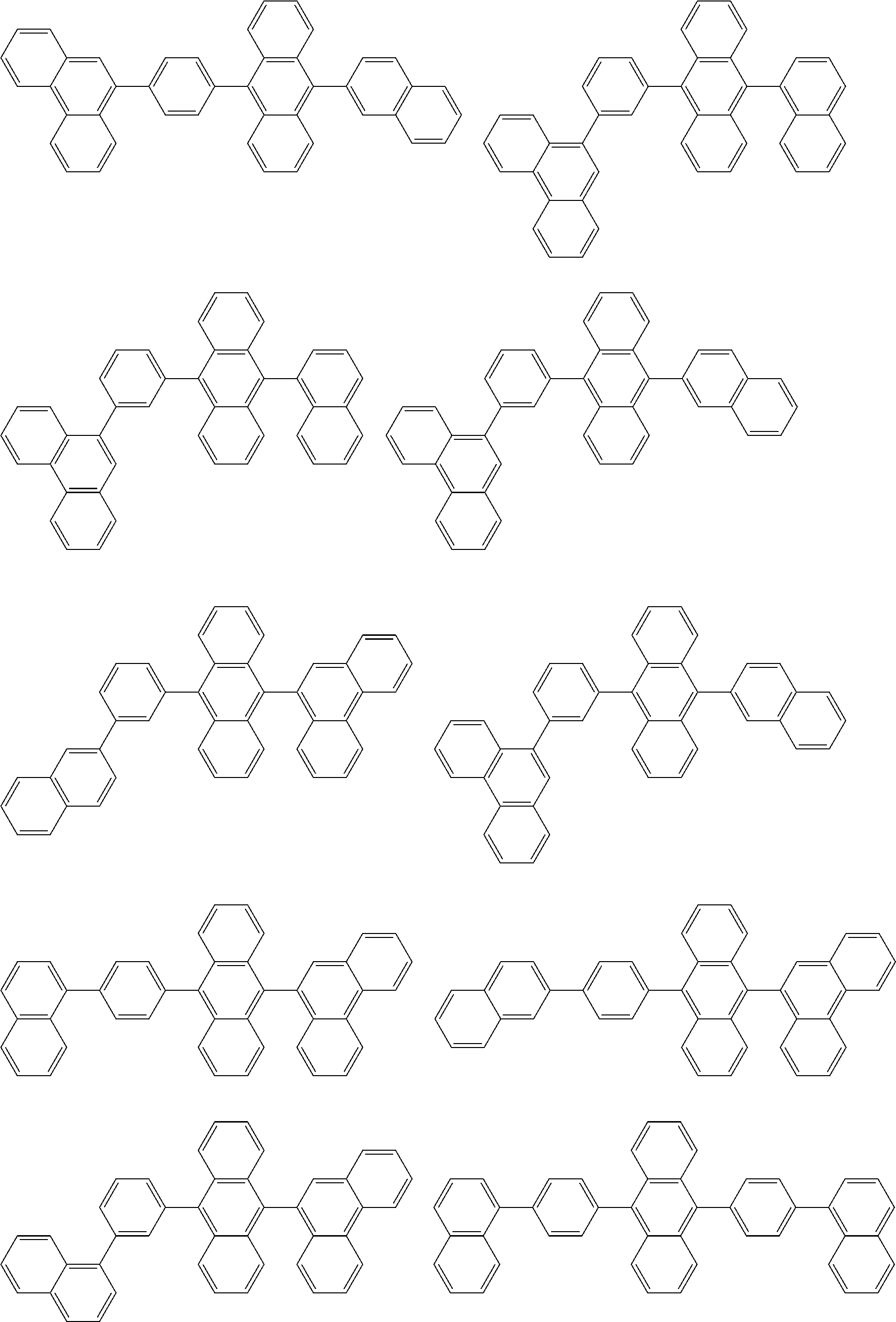

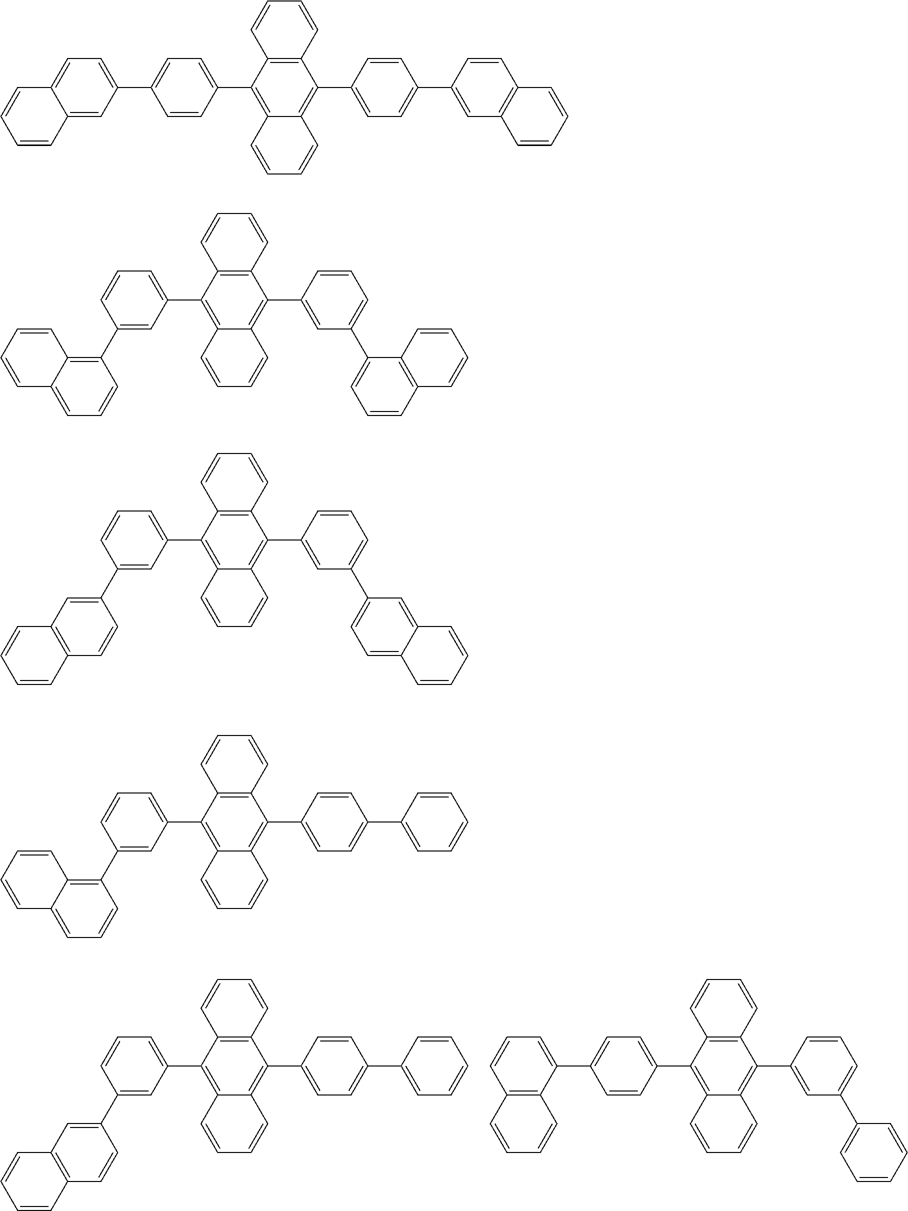

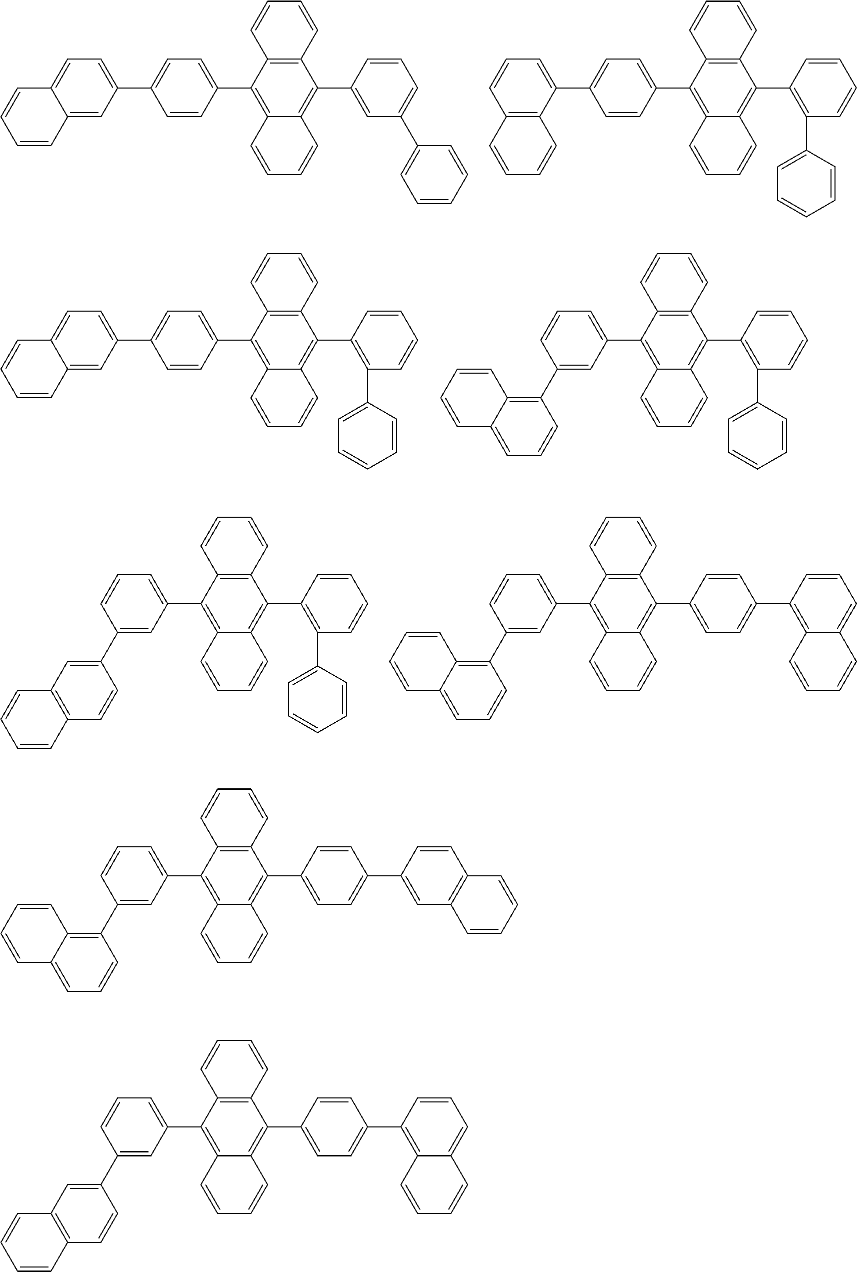

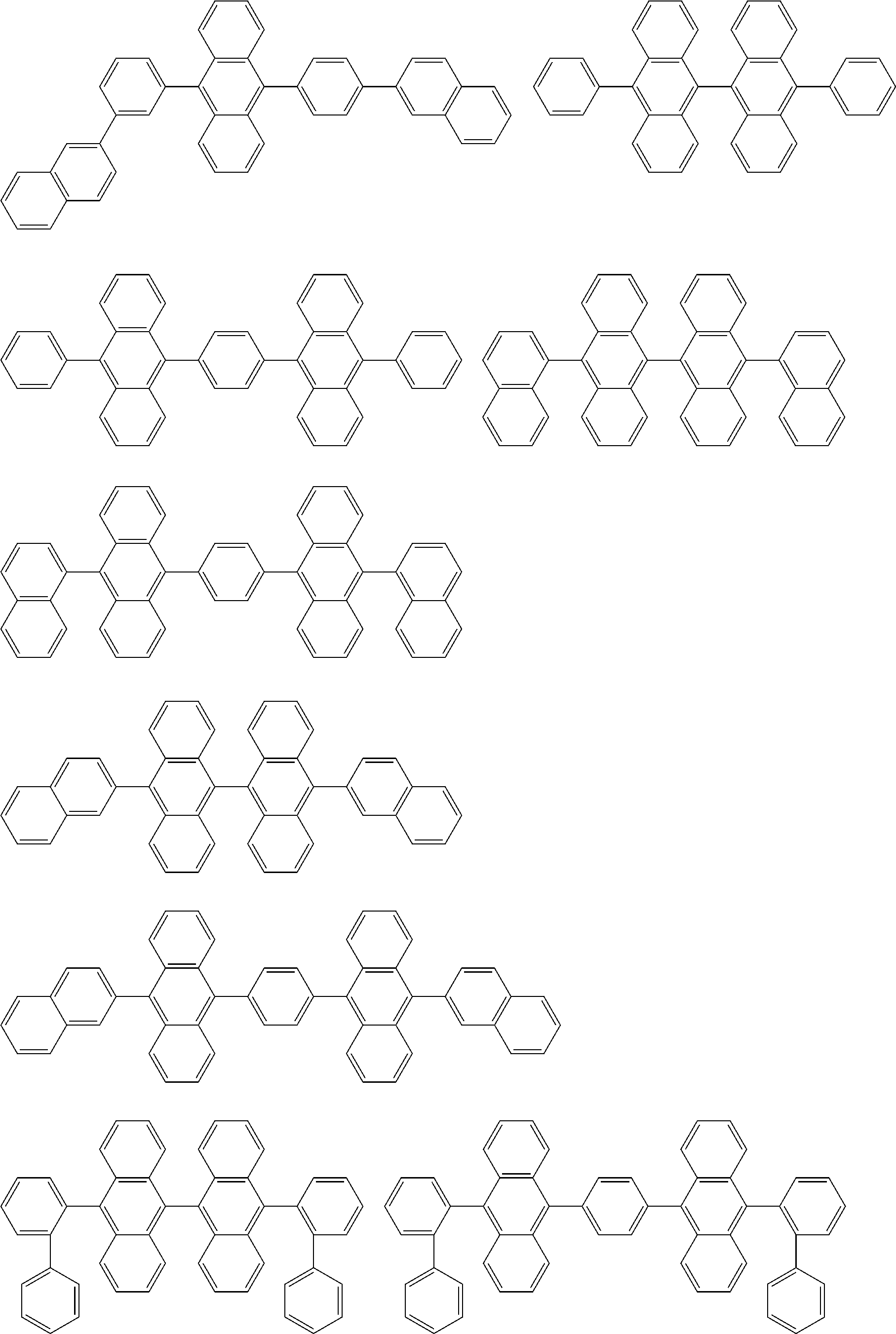

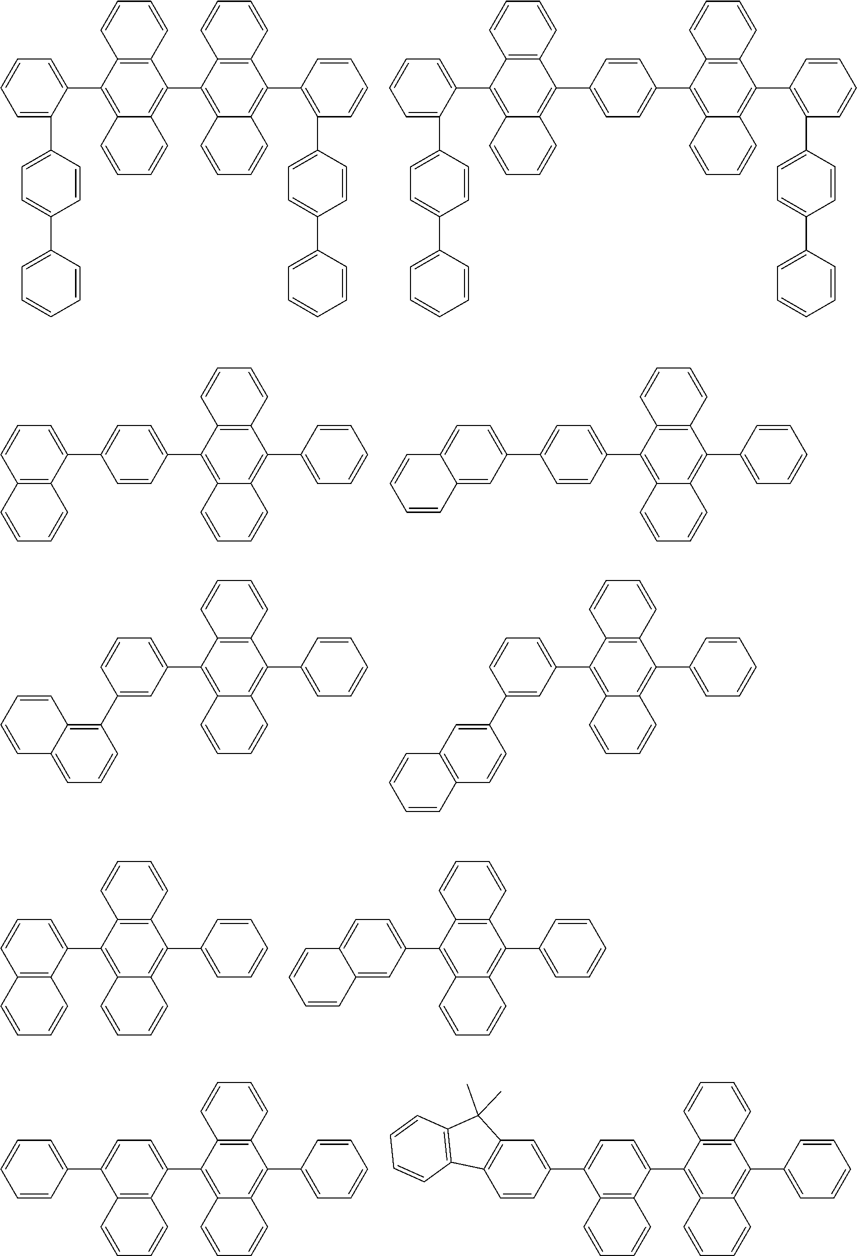

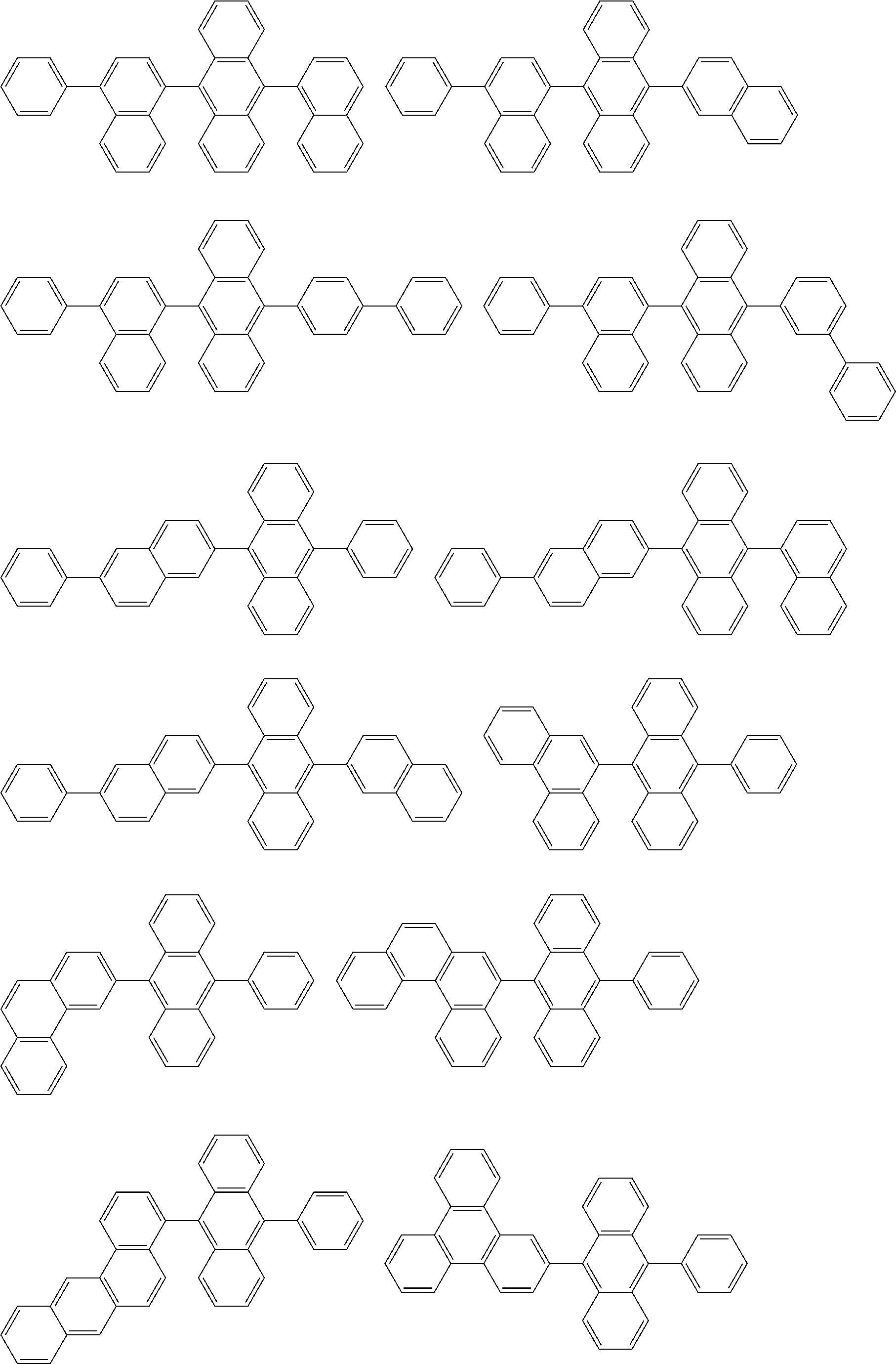

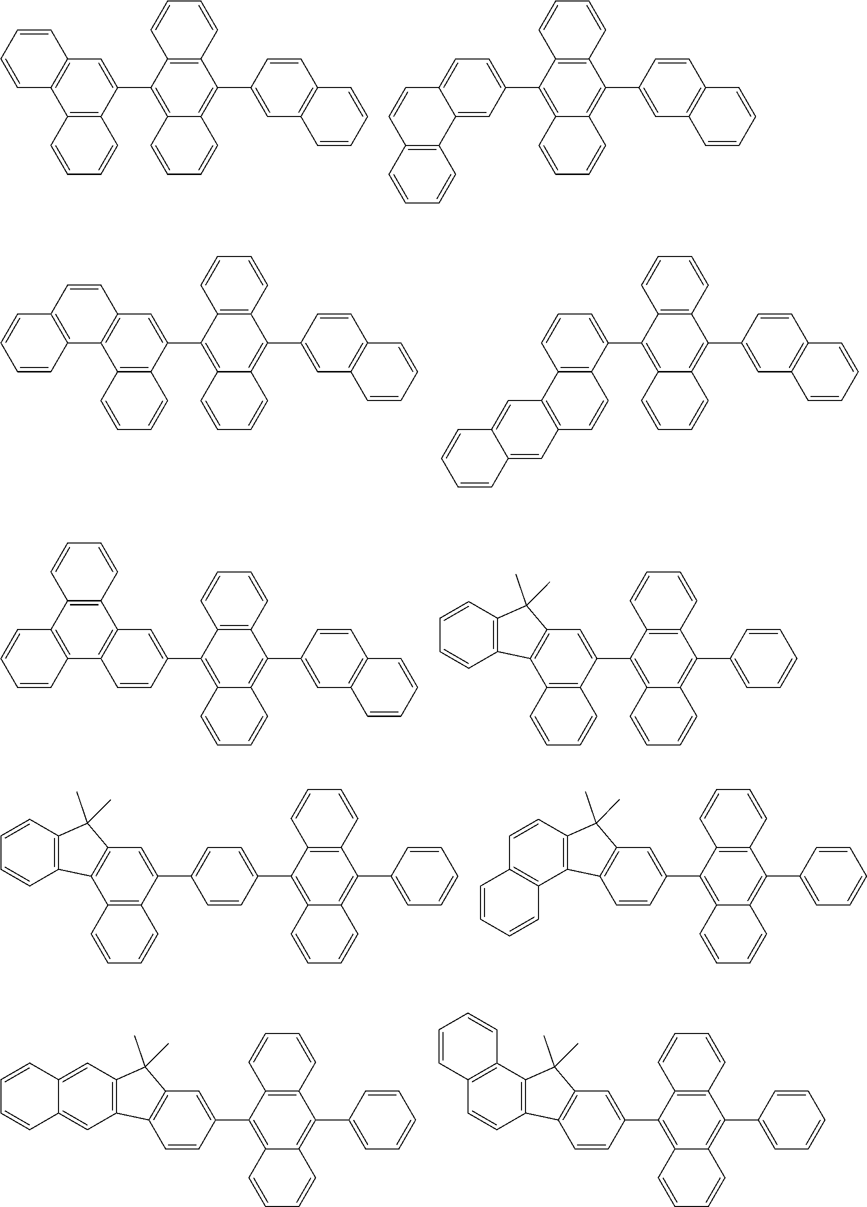

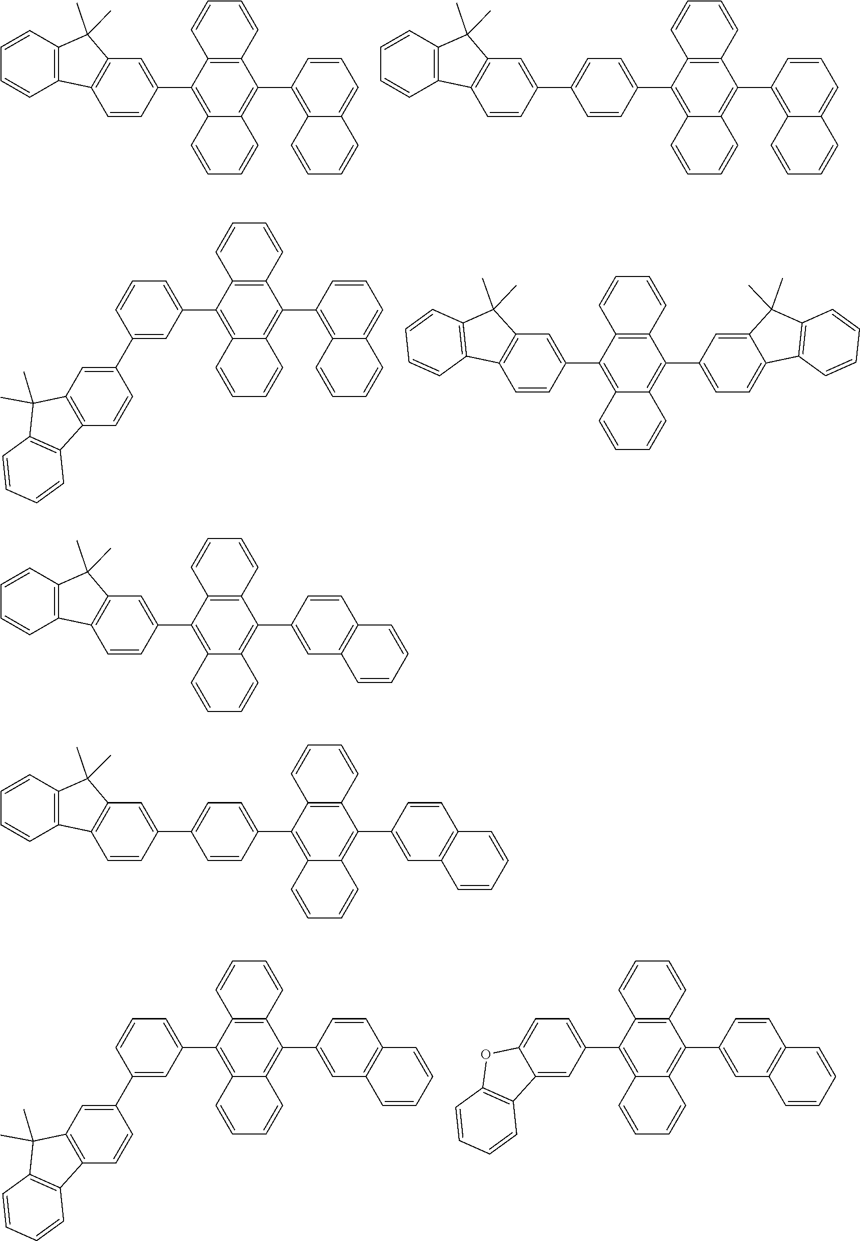

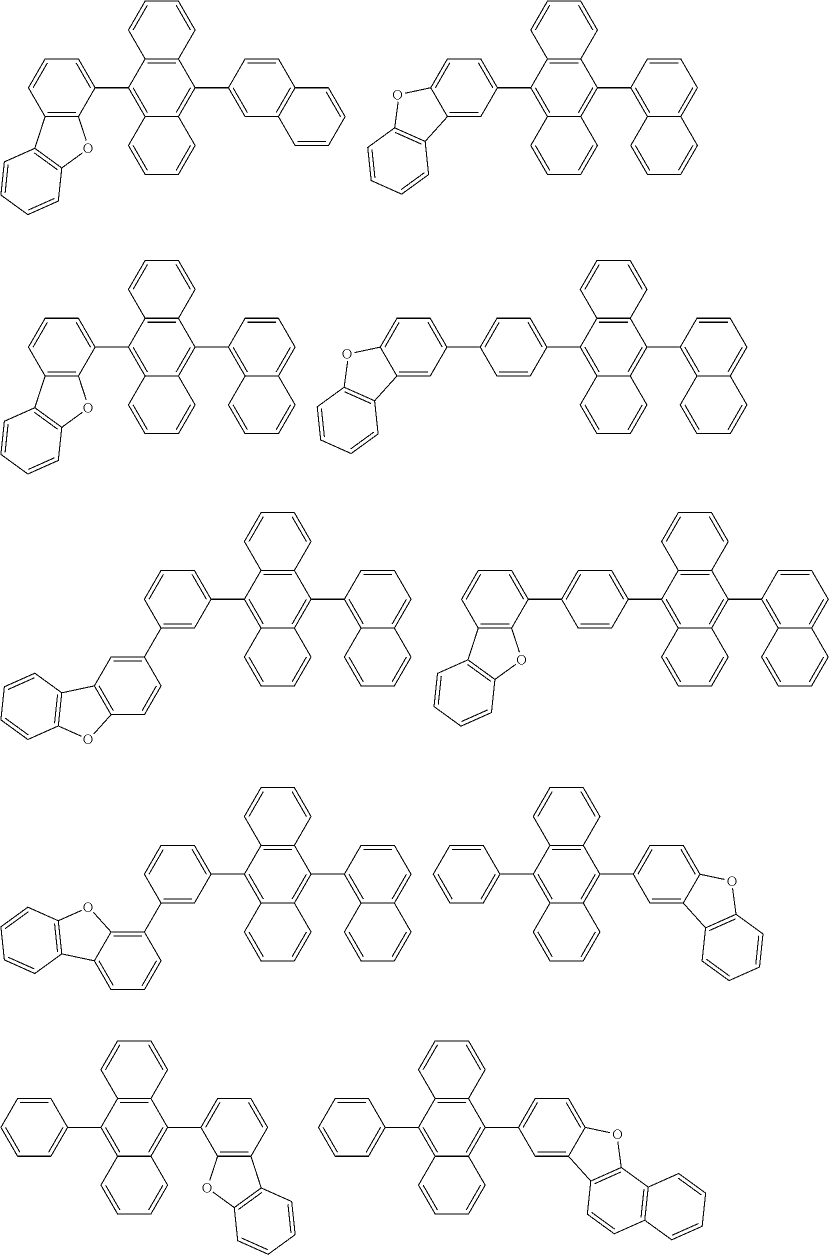

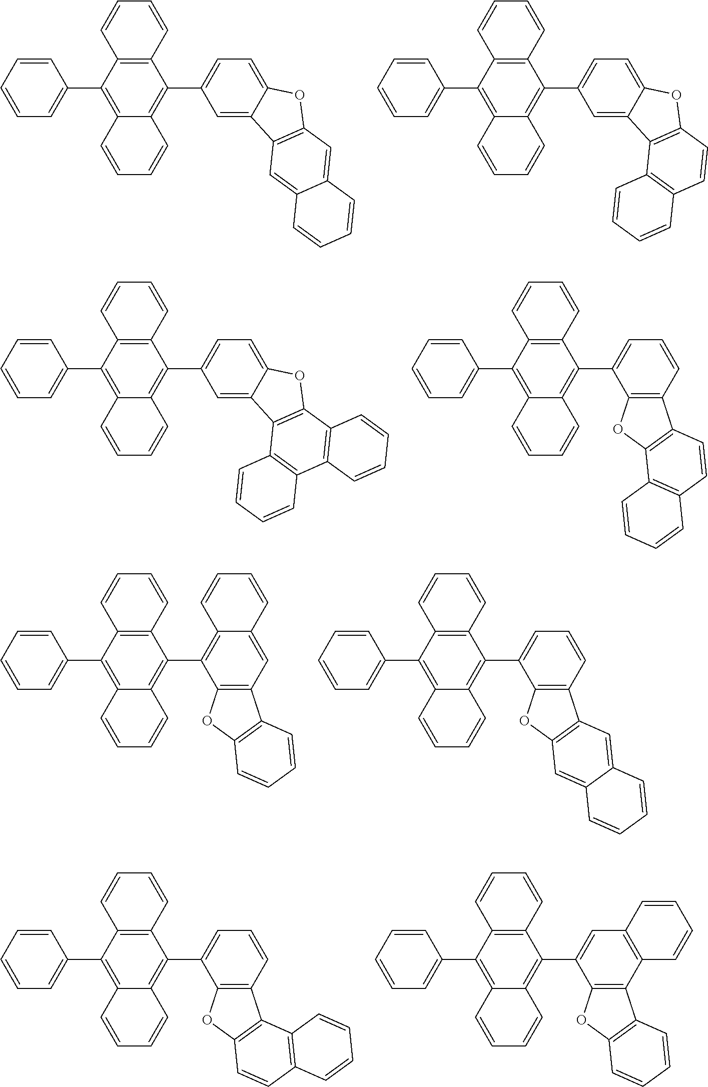

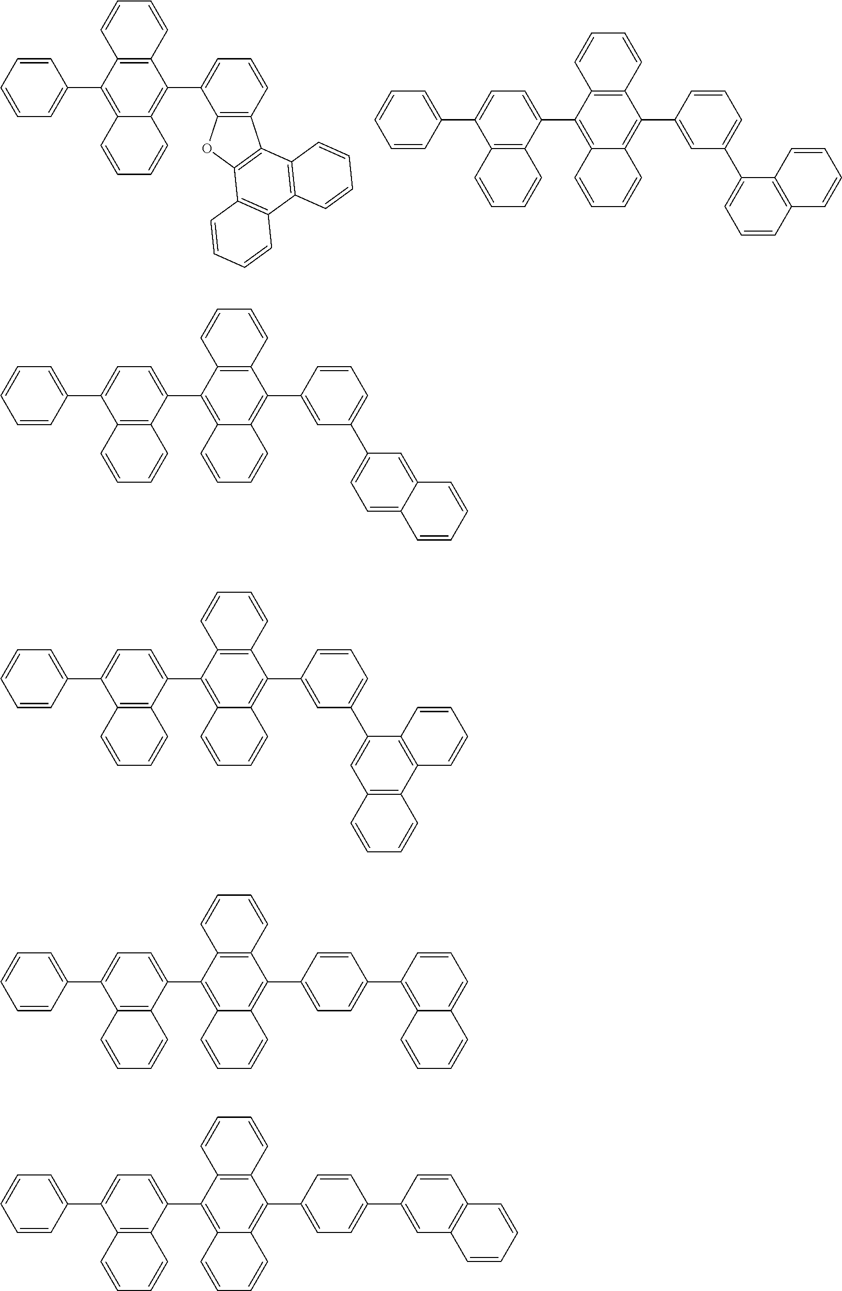

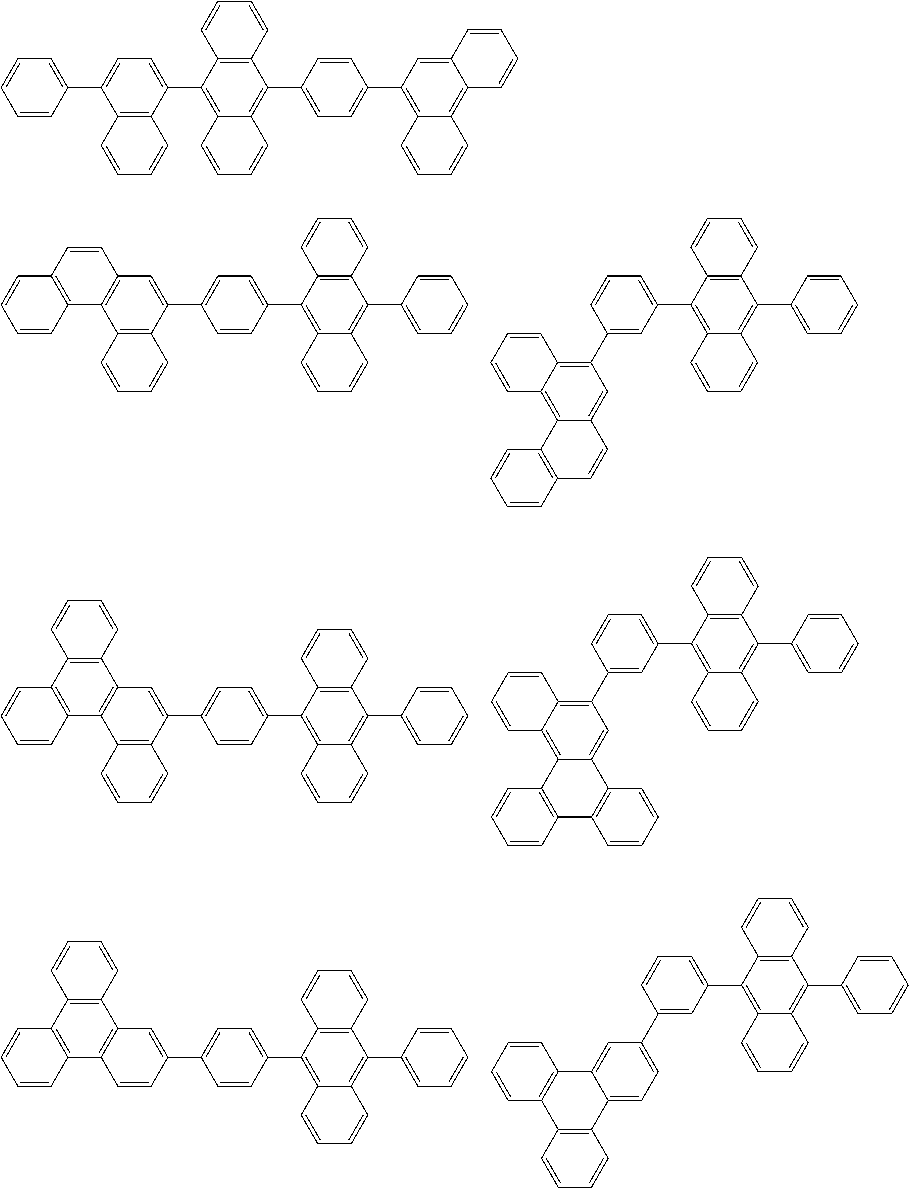

In one aspect of the organic EL device according to the present invention, when the light emitting layer includes a first light emitting layer and a second light emitting layer, at least one of components constituting the first light emitting layer is different from components constituting the second light emitting layer. For example, a mode in which the dopant material contained in the first light emitting layer is different from the dopant material contained in the second light emitting layer, or a mode in which the host material contained in the first light emitting layer is different from the host material contained in the second light emitting layer may be exemplified.

In the organic EL device of the present invention, the light emitting layer may contain a light-emitting compound that exhibits fluorescence emission with a main peak wavelength of 500 nm or less (hereinafter, also simply referred to as a ““fluorescence emitting compound”).

The method of measuring the main peak wavelength of the compound is as follows. A 5 μmon toluene solution of a compound as a measurement target is prepared and placed in a quartz cell, and then the emission spectrum (emission intensity is set for the vertical axis, and wavelength is set for the horizontal axis) of this test sample is measured at room temperature (300 K). The emission spectrum can be measured by a fluorescence spectrophotometer (device name: F-7000) manufactured by Hitachi High-Tech Science corporation. The emission spectrum measuring device is not limited to the device used here.

In the emission spectrum, the peak wavelength of the emission spectrum at which the emission intensity is maximum is set as the main peak wavelength. In this specification, the main peak wavelength may be referred to as a fluorescence emission main peak wavelength (FL-peak) in some cases.

The fluorescence emitting compound may be the dopant material, or may be the host material.

When the light emitting layer is a single layer, only one of the dopant material and the host material may be the fluorescence emitting compound, or both may be the fluorescence emitting compound.

Further, when the light emitting layer includes a first light emitting layer (anode side) and a second light emitting layer (cathode side), only one of the first light emitting layer and the second light emitting layer may contain the fluorescence emitting compound, or both light emitting layers may contain the fluorescence emitting compound. When the first light emitting layer contains the fluorescence emitting compound, only one of the dopant material and the host material contained in the first light emitting layer may be the fluorescence emitting compound, or both may be the fluorescence emitting compound. Further, when the second light emitting layer contains the fluorescence emitting compound, only one of the dopant material and the host material contained in the second light emitting layer may be the fluorescence emitting compound, or both may be the fluorescence emitting compound.

Electron Transporting Layer

The electron transporting layer is a layer containing a material having a high electron transporting capability (an electron transporting material), and is formed between the light emitting layer and the cathode, or between the light emitting layer and the electron injecting layer if the electron injecting layer exists.

The electron transporting layer may have a single-layer structure, or may have a multi-layer structure including two or more layers. For example, the electron transporting layer may have a two-layer structure including a first electron transporting layer (anode side) and a second electron transporting layer (cathode side). In an aspect of the present invention, the electron transporting layer with the single-layer structure is preferably adjacent to the light emitting layer, and the electron transporting layer closest to the anode in the multi-layer structure, for example, the first electron transporting layer in the two-layer structure, is preferably adjacent to the light emitting layer. In another aspect of the present invention, a hole blocking layer to be described below may be interposed between the electron transporting layer with the single-layer structure and the light emitting layer, or between the light emitting layer and the electron transporting layer closest to the light emitting layer in the multi-layer structure.

For the electron transporting layer, for example,
(1) a metal complex such as an aluminum complex, a beryllium complex, or a zinc complex,
(2) a heteroaromatic compound such as an imidazole derivative, a benzoimidazole derivative, an azine derivative, a carbazole derivative, or a phenanthroline derivative, and
(3) a high molecular compound can be used.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq2), bis(2-methyl-8-quinolinolato) (4-phenylphenolato)aluminum(III) (abbreviation: BAlq), bis(8-quinolinolato)zinc(II) (abbreviation: Znq), bis[2-(2-benzooxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzooxazole-2-yl)stilbene (abbreviation: BzOs).

Examples of the high molecular compound include poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy).

The material is a material having an electron mobility of 10⁻⁶ cm²/Vs or more. A material other than the above may be used for the electron transporting layer as long as the material has an electron transporting capability higher than a hole transporting capability.

Electron Injecting Layer

The electron injecting layer is a layer containing a material with a high electron injection capability. For the electron injecting layer, alkali metals such as lithium (Li), and cesium (Cs), alkaline earth metals such as magnesium (Mg), calcium (Ca), and strontium (Sr), rare earth metals such as europium (Eu), and ytterbium (Yb), and compounds containing these metals can be used. Examples of such compounds include alkali metal oxides, alkali metal halides, alkali metal-containing organic complexes, alkaline earth metal oxides, alkaline earth metal halides, alkaline earth metal-containing organic complexes, rare earth metal oxides, rare earth metal halides, and rare earth metal-containing organic complexes. These compounds can also be mixed and then used.

Besides, a material having an electron transporting capability, in which an alkali metal, an alkaline earth metal, or a compound thereof is contained, specifically, Alq in which magnesium (Mg) is contained, may be used. Further, in this case, electron injecting from the cathode can be more efficiently performed.

Otherwise, for the electron injecting layer, a composite material obtained by mixing an organic compound with an electron donor may be used. Such a composite material is excellent in the electron injection capability and the electron transporting capability because the organic compound accepts electrons from the electron donor. In this case, as for the organic compound, a material excellent in transporting accepted electrons is preferred, and specifically, for example, the above described materials (a metal complex, a heteroaromatic compound or the like) constituting the electron transporting layer can be used. The electron donor only has to be a material exhibiting an electron donation capability for the organic compound. Specifically, alkali metals, alkaline earth metals and rare earth metals are preferred, and lithium, cesium, magnesium, calcium, erbium, ytterbium, etc. may be exemplified. Further, alkali metal oxide or alkaline earth metal oxide is preferred, and lithium oxide, calcium oxide, barium oxide, etc. may be exemplified. Further, Lewis bases such as magnesium oxide can also be used. Further, organic compounds such as tetrathiafulvalene (abbreviation: TTF) can also be used.

Cathode

For the cathode, it is preferable to use a metal, an alloy, an electrically conductive compound, a mixture thereof or the like which has a small work function (specifically 3.8 eV or less). Specific examples of these cathode materials include elements belonging to Group 1 or Group 2 of the periodic table of elements, i.e., alkali metals such as lithium (Li) or cesium (Cs), alkaline earth metals such as magnesium (Mg), calcium (Ca), and strontium (Sr), and alloys containing these (e.g., MgAg, AILi), and rare earth metals such as europium (Eu) and ytterbium (Yb) and alloys containing these.

When the cathode is formed by using the alkali metals, the alkaline earth metals, or the alloys containing these, a vacuum deposition method or a sputtering method can be used. Further, when a silver paste or the like is used, a coating method, an inkjet method or the like can be used.

By providing the electron injecting layer, regardless of the magnitude of the work function, it is possible to form the cathode by using various conductive materials such as Al, Ag, ITO, graphene, and indium oxide-tin oxide containing silicon or silicon oxide. These conductive materials can be deposited by using a sputtering method or an inkjet method, a spin coating method, etc.

Insulating Layer

In the organic EL device, since an electric field is applied to an ultra-thin film, pixel defects are likely to occur due to leaks or short-circuiting. In order to prevent this, an insulating layer formed of an insulating thin film layer may be inserted between a pair of electrodes.

As for the material used for the insulating layer, for example, aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, vanadium oxide etc. may be exemplified. Further, a mixture or a laminate of these may also be used.

Space Layer

The space layer is, for example, a layer provided between the fluorescence emitting layer and the phosphorescence emitting layer for the purpose of preventing excitons generated in the phosphorescence emitting layer from diffusing into the fluorescence emitting layer or adjusting a carrier balance, in a case where the fluorescence emitting layer and the phosphorescence emitting layer are stacked. The space layer can also be provided between phosphorescence emitting layers.

Since the space layer is provided between the light emitting layers, a material having both an electron transporting capability and a hole transporting capability is preferred. Further, the triplet energy is preferably 2.6 eV or more in order to prevent the diffusion of triplet energy in the adjacent phosphorescence emitting layer. As for the material used for the space layer, the same as those used for the above described hole transporting layer may be exemplified.

Blocking layer The blocking layer such as the electron blocking layer, the hole blocking layer, or the exciton blocking layer may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers, and confining the excitons within the light emitting layer.

Each layer of the organic EL device can be formed by a conventionally known vapor deposition method, a coating method, etc. For example, formation can be performed by a known method using a vapor deposition method, such as a vacuum deposition method or a molecular beam deposition method (MBE method), or a coating method in which a solution of a compound for forming a layer is used, such as a dipping method, a spin-coating method, a casting method, a bar-coating method, or a roll-coating method.

The film thickness of each layer is not particularly limited, but is usually 5 nm to 10 μm, more preferably 10 nm to 0.2 μm because in general, when the film thickness is too small, defects such as pinholes are likely to occur, and conversely, when the film thickness is too large, a high driving voltage is required and the efficiency becomes poor.

In the organic EL device having the hole transporting layer with the two-layer structure or the three-layer structure in the present invention, the sum of the thickness of the first hole transporting layer and the thickness of the second hole transporting layer is preferably 30 nm or more, and 150 nm or less, more preferably, 40 nm or more, and 130 nm or less.

Further, in one aspect of the organic EL device of the present invention, the thickness of the second hole transporting layer is preferably 5 nm or more, more preferably 20 nm or more, further preferably 25 nm or more, particularly preferably 35 nm or more, and is preferably 100 nm or less.

Further, in one aspect of the organic EL device of the present invention, the thickness of the hole transporting layer adjacent to the light emitting layer is preferably 5 nm or more, more preferably 20 nm or more, further preferably 25 nm or more, particularly preferably 30 nm or more, and is preferably 100 nm or less.

Further, in one aspect of the organic EL device of the present invention, the film thickness D1 of the first hole transporting layer and the film thickness D2 of the second hole transporting layer satisfy the relationship of 0.3<D2/D1<4.0, and preferably satisfy the relationship of 0.5<D2/D1<3.5, more preferably satisfy the relationship of 0.75<D2/D1<3.0.

As preferred embodiments of the organic EL device of the present invention, for example,

(1) an organic EL device having a hole transporting layer with a two-layer structure
a first embodiment in which the second hole transporting layer contains the invention compound, and the first hole transporting layer does not contain the invention compound;
a second embodiment in which both the first hole transporting layer and the second hole transporting layer contain the invention compound; and
a third embodiment in which the first hole transporting layer contains the invention compound, and the second hole transporting layer does not contain the invention compound; and

(2) an organic EL device having a hole transporting layer with a three-layer structure
a fourth embodiment in which the first hole transporting layer contains the invention compound, and the second and third hole transporting layers do not contain the invention compound;
a fifth embodiment in which the second hole transporting layer contains the invention compound, and the first and third hole transporting layers do not contain the invention compound;
a sixth embodiment in which the third hole transporting layer contains the invention compound, and the first and second hole transporting layers do not contain the invention compound;
a seventh embodiment in which the first and second hole transporting layers contain the invention compound, and the third hole transporting layer does not contain the invention compound;
an eighth embodiment in which the first and third hole transporting layers contain the invention compound, and the second hole transporting layer does not contain the invention compound;
a ninth embodiment in which the second and third hole transporting layers contain the invention compound, and the first hole transporting layer does not contain the invention compound; and
a tenth embodiment in which all of the first to third hole transporting layers contain the invention compound; may be exemplified.

Electronic Device

The organic EL device can be used for electronic device, as display components of organic EL panel modules, etc., display devices of televisions, mobile phones, personal computers, etc., and light emitting devices of lightings, vehicular lamps, etc.

### EMBODIMENTS

Hereinafter, the present invention will be described in more detail by using Examples, but the present invention is not limited to the following Examples.

Invention compounds used for producing organic EL devices in Examples 1 to 3

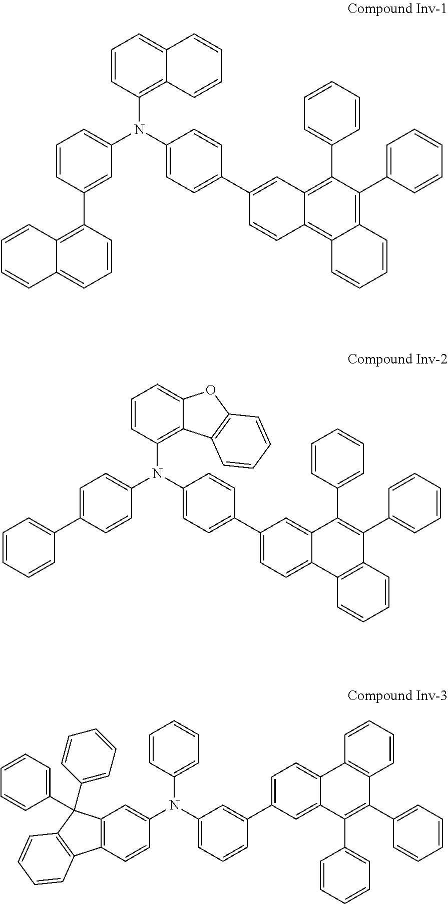 [SMILES: c1ccc(-c2c(-c3ccccc3)c3cc(-c4ccc(N(c5cccc(-c6cccc7ccccc67)c5)c5cccc6ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)cc2)cc1 ; c1ccc(-c2c(-c3ccccc3)c3cc(-c4cccc(N(c5ccccc5)c5ccc6c(c5)C(c5ccccc5)(c5ccccc5)c5ccccc5-6)c4)ccc3c3ccccc23)cc1]

A comparative compound used for producing an organic EL device in Comparative Example 1

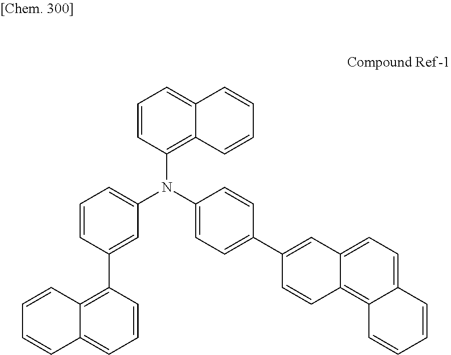 [SMILES: c1cc(-c2cccc3ccccc23)cc(N(c2ccc(-c3ccc4c(ccc5ccccc54)c3)cc2)c2cccc3ccccc23)c1]

Other compounds used for producing the organic EL devices in Examples 1, 2, and 3 and Comparative Example 1

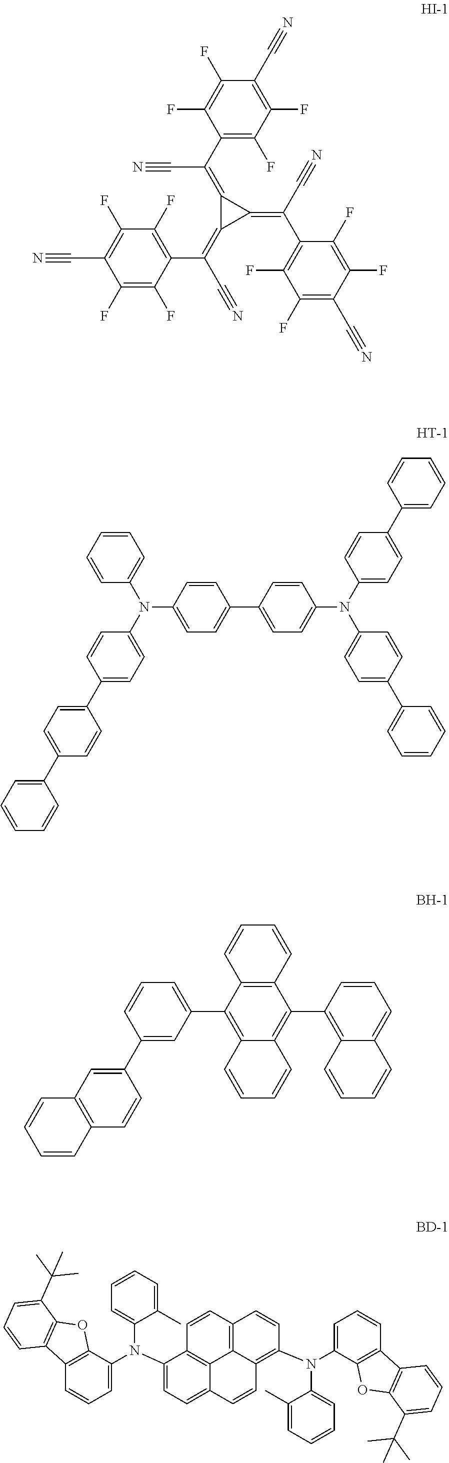

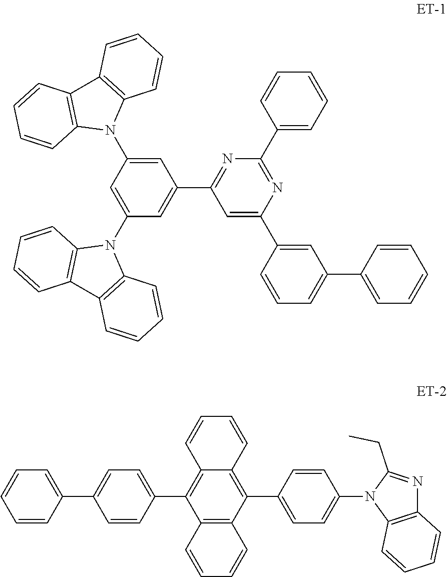 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Production of Organic EL Device

### Example 1

A glass substrate provided with an ITO transparent electrode (anode) (manufactured by GEOMATEC CO., Ltd., 25 mm×75 mm×1.1 mm) was ultrasonically cleaned in isopropyl alcohol for 5 min, and then was subjected to UV ozone cleaning for 30 min. The film thickness of the ITO was set as 130 nm.

The cleaned glass substrate provided with the ITO transparent electrode was mounted on a substrate holder of a vacuum deposition device.

First, the compound HT-1 and the compound HI-1 were co-deposited on the top surface formed with the transparent electrode such that the transparent electrode is covered, and then a hole injecting layer with a film thickness of 10 nm was formed. The mass ratio (HT-1:HI-1) of the compound HT-1 to the compound HI-1 was 97:3.

Next, on the hole injecting layer, the compound HT-1 was deposited to form a first hole transporting layer with a film thickness of 80 nm.

Next, on the first hole transporting layer, the invention compound Inv-1 was deposited to form a second hole transporting layer with a film thickness of 10 nm.

Next, on the second hole transporting layer, the compound BH-1 (host material) and the compound BD-1 (dopant material) were co-deposited to form a light emitting layer with a film thickness of 25 nm. The mass ratio (BH-1:BD-1) of the compound BH-1 to the compound BD-1 was 96:4.

Next, on the light emitting layer, the compound ET-1 was deposited to form a first electron transporting layer with a film thickness of 10 nm.

Next, on the first electron transporting layer, the compound ET-2 was deposited to form a second electron transporting layer with a film thickness of 15 nm.

Next, on the second electron transporting layer, LiF was deposited to form an electron injecting electrode with a film thickness of 1 nm.

Then, on the electron injecting electrode, a metal Al was deposited to form a metal cathode with a film thickness of 50 nm.

The layer structure of the organic EL device obtained in this manner is illustrated below.

ITO(130)/HT-1: HI-1=97: 3(10)/HT-1(80)/Inv-1(10)/BH-1: BD-1=96: 4(25)/ET-1(10)/ET-2(15)/LiF(1)/A1(50)

In the layer structure, numbers in brackets indicate film thicknesses (nm), and ratios indicate mass ratios.

### Examples 2 and 3

Each organic EL device was produced in the same manner as in Example 1 except that the invention compound Inv-2 (Example 2), and the invention compound Inv-3 (Example 3) were used instead of the invention compound Inv-1.

### Comparative Example 1

An organic EL device was produced in the same manner as in Example 1 except that the comparative compound Ref-1 was used instead of the invention compound Inv-1.

Evaluation of Organic EL Device

Measurement of External Quantum Efficiency (EQE)

The obtained organic EL device was driven with a DC constant current at room temperature at a current density of 10 mA/cm². The luminance was measured by using a luminance meter (spectroradiometer CS-1000 manufactured by Minolta), and the external quantum efficiency (%) was determined from the result. The results are noted in Table 1.

TABLE 1

| | Second Hole | |
|---|---|---|
| | transporting | External Quantum |
| | Layer Material | Efficiency (%) |
| Example 1 | Inv-1 | 10.64 |
| Example 2 | Inv-2 | 10.78 |
| Example 3 | Inv-3 | 10.62 |
| Comparative Example 1 | Ref-1 | 9.88 |

As is clear from the results in Table 1, the invention compound Inv-1, the invention compound Inv-2, and the invention compound Inv-3 provide organic EL devices with higher external quantum efficiencies than the comparative compound Ref-1.

### Invention Compounds Synthesized in Synthesis Examples

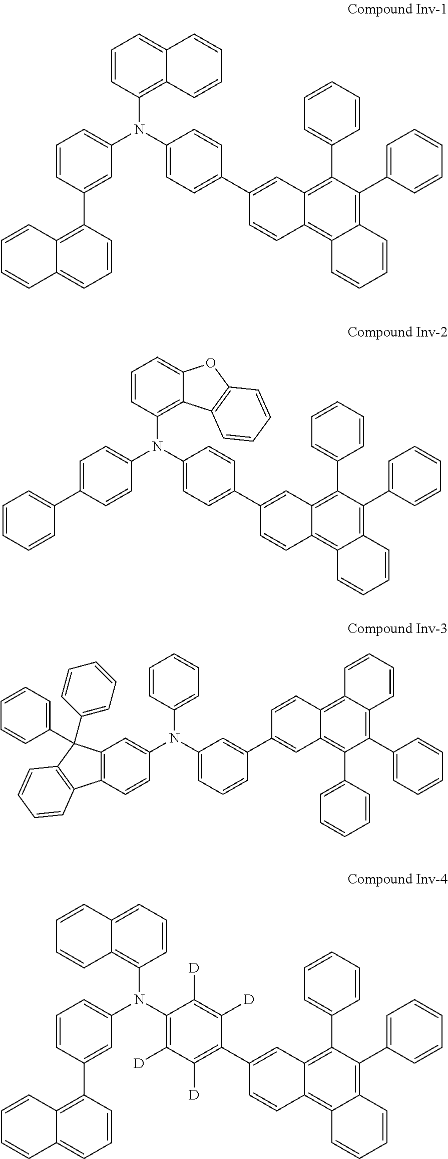 [SMILES: c1ccc(-c2c(-c3ccccc3)c3cc(-c4ccc(N(c5cccc(-c6cccc7ccccc67)c5)c5cccc6ccccc56)cc4)ccc3c3ccccc23)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)cc2)cc1 ; c1ccc(-c2c(-c3ccccc3)c3cc(-c4cccc(N(c5ccccc5)c5ccc6c(c5)C(c5ccccc5)(c5ccccc5)c5ccccc5-6)c4)ccc3c3ccccc23)cc1 ; [2H]c1c([2H])c(N(c2cccc(-c3cccc4ccccc34)c2)c2cccc3ccccc23)c([2H])c([2H])c1-c1ccc2c(c1)c(-c1ccccc1)c(-c1ccccc1)c1ccccc12]

### Intermediate Synthesis Example 1: Synthesis of Intermediate a

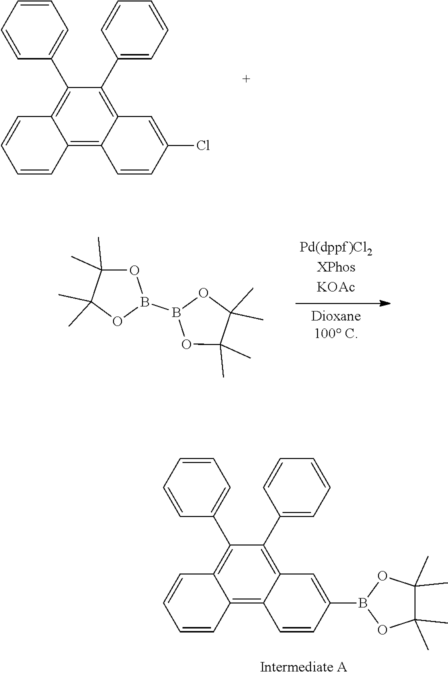

Under an argon atmosphere, a mixture of 2-chloro-9,10-diphenylphenanthrene (10.95 g, 30.00 mmol), 4,4,4′,4′,5,5,5′,5′-octamethyl-2,2′-bi-1,3,2-dioxaborolane (9.14 g, 36.00 mmol), [1,1′-bis(diphenylphosphino)ferrocene]dichloropalladium (II) (0.490 g, 0.600 mmol), XPhos (1.15 g, 2.40 mmol), potassium acetate (8.83 g, 90.00 mmol), and 1,4-dioxane (300 mL), which was synthesized in the same manner as described in Angew. Chem. Int. Ed. 2017, Vol. 56, p. 1361-1364, was stirred at 100° C. for 3 h. The reaction solution was cooled to room temperature, and concentrated under reduced pressure. The resulting residue was purified by silica gel column chromatography to obtain Intermediate A as a white solid (12.5 g). The yield was 91%.

### Intermediate Synthesis Example 2: Synthesis of Intermediate B

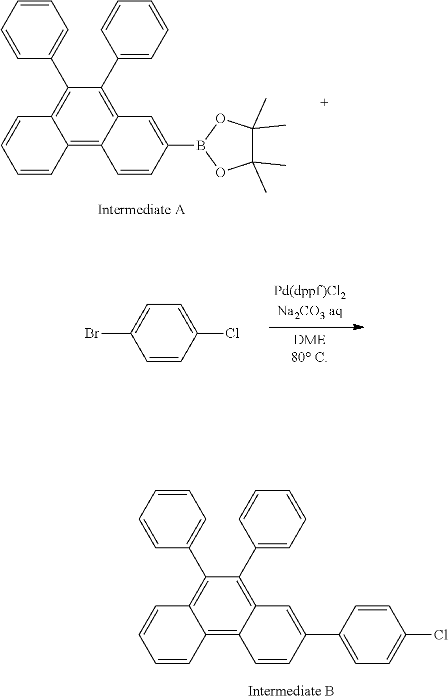 [SMILES: CC1(C)OB(c2ccc3c(c2)c(-c2ccccc2)c(-c2ccccc2)c2ccccc23)OC1(C)C ; Clc1ccc(Br)cc1 ; Clc1ccc(-c2ccc3c(c2)c(-c2ccccc2)c(-c2ccccc2)c2ccccc23)cc1]

Under an argon atmosphere, a mixture of Intermediate A (12.5 g, 27.4 mmol), 1-bromo-4-chlorobenzene (5.24 g, 27.4 mmol), [1,1′-bis(diphenylphosphino)ferrocene]dichloropalladium(II) (0.661 g, 0.904 mmol), sodium carbonate (8.71 g, 82.0 mmol), DME (137 mL) and water (41.1 mL) was stirred at 80° C. for 7 h. The reaction solution was cooled to room temperature, and concentrated under reduced pressure. The resulting residue was purified by silica gel column chromatography to obtain Intermediate B as a white solid (11.51 g). The yield was 95%.

### Intermediate Synthesis Example 3: Synthesis of Intermediate C

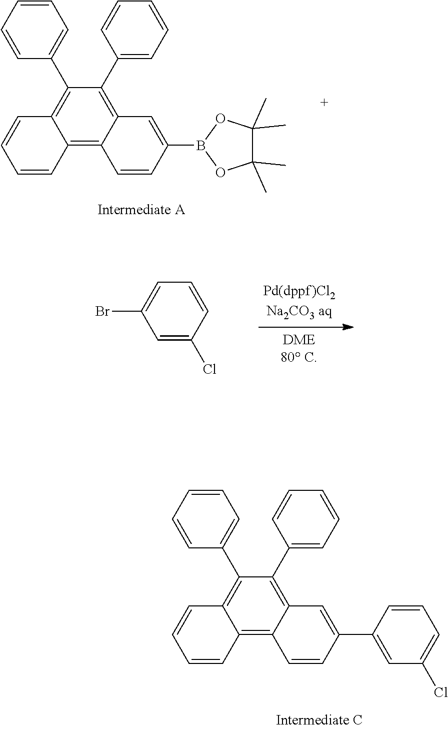 [SMILES: CC1(C)OB(c2ccc3c(c2)c(-c2ccccc2)c(-c2ccccc2)c2ccccc23)OC1(C)C ; Clc1cccc(Br)c1 ; Clc1cccc(-c2ccc3c(c2)c(-c2ccccc2)c(-c2ccccc2)c2ccccc23)c1]

Under an argon atmosphere, a mixture of Intermediate A (8.22 g, 18.0 mmol), 1-bromo-3-chlorobenzene (3.45 g, 18.0 mmol), [1,1′ bis(diphenylphosphino)ferrocene]dichloropalladium (II) (0.435 g, 0.594 mmol), sodium carbonate (5.72 g, 54.0 mmol), DME (90 mL), and water (27 mL) was stirred at 80° C. for 7 h. The reaction solution was cooled to room temperature, and concentrated under reduced pressure. The resulting residue was purified by silica gel column chromatography to obtain Intermediate C as a white solid (7.28 g). The yield was 92%.

### Intermediate Synthesis Example 4: Synthesis of Intermediate D

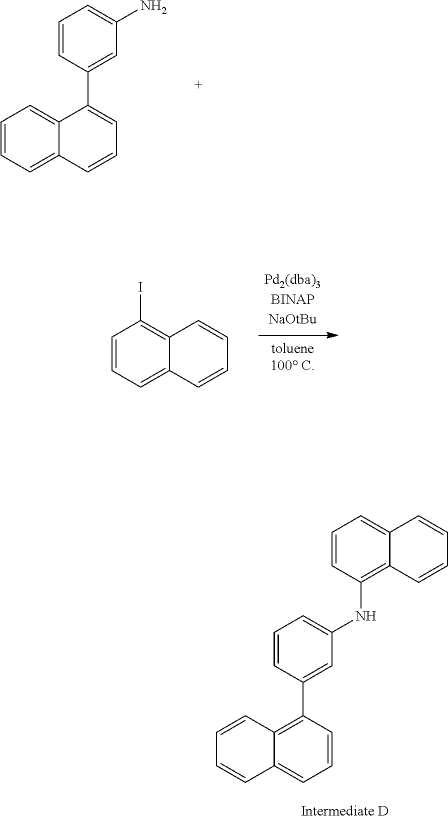 [SMILES: Nc1cccc(-c2cccc3ccccc23)c1 ; Ic1cccc2ccccc12 ; c1cc(Nc2cccc3ccccc23)cc(-c2cccc3ccccc23)c1]

Under an argon atmosphere, a mixture of 3-(naphthalene-1-yl)aniline (5.68 g, 25.9 mmol), 1-iodinenaphthalene (6.58 g, 25.9 mmol), tris(dibenzylideneacetone)dipalladium(0) (0.474 g, 0.518 mmol), BINAP (0.645 g, 1.04 mmol), sodium-t-butoxide (2.74 g, 28.5 mmol) and toluene (130 mL) was stirred at 100° C. for 3 h. The reaction solution was cooled to room temperature, and concentrated under reduced pressure. The resulting residue was purified by silica gel column chromatography to obtain Intermediate D as a white solid (7.49 g). The yield was 84%.

### Intermediate Synthesis Example 5: Synthesis of Intermediate E

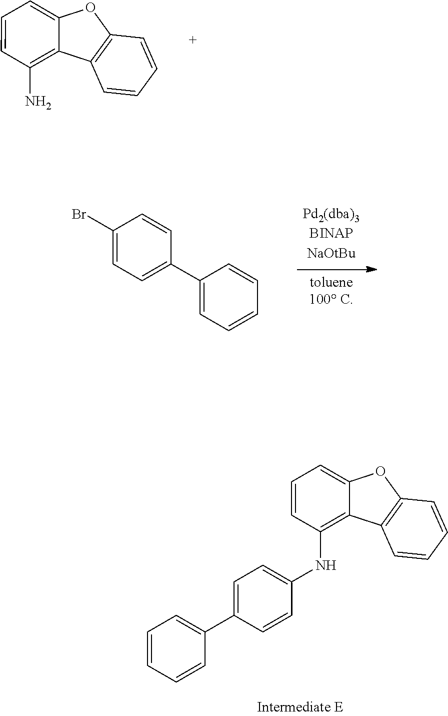 [SMILES: Nc1cccc2oc3ccccc3c12 ; Brc1ccc(-c2ccccc2)cc1 ; c1ccc(-c2ccc(Nc3cccc4oc5ccccc5c34)cc2)cc1]

Under an argon atmosphere, a mixture of dibenzo[b,d]furan-1-amine (2.50 g, 13.65 mmol), 4-bromo-1,1′-biphenyl (3.18 g, 13.65 mmol), tris(dibenzylideneacetone)dipalladium(0) (0.250 g, 0.273 mmol), BINAP (0.340 g, 0.546 mmol), sodium-t-butoxide (1.84 g, 19.10 mmol) and toluene (91 mL) was stirred at 100° C. for 3 h. The reaction solution was cooled to room temperature, and concentrated under reduced pressure. The resulting residue was purified by silica gel column chromatography to obtain Intermediate E as a white solid (4.03 g). The yield was 88%.

### Intermediate Synthesis Example 5: Synthesis of Intermediate F

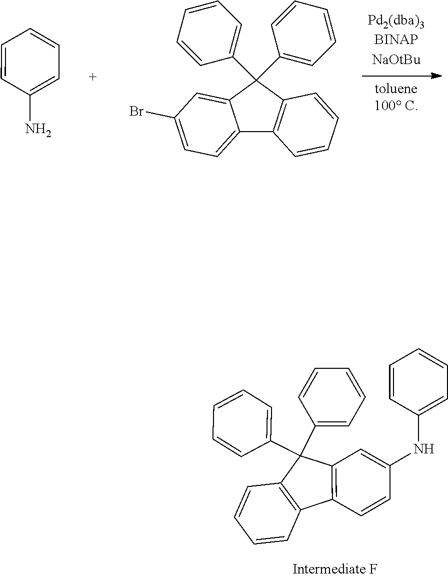 [SMILES: Nc1ccccc1 ; Brc1ccc2c(c1)C(c1ccccc1)(c1ccccc1)c1ccccc1-2 ; c1ccc(Nc2ccc3c(c2)C(c2ccccc2)(c2ccccc2)c2ccccc2-3)cc1]

Under an argon atmosphere, a mixture of aniline (5.59 g, 60.0 mmol), 2-bromo-9,9-diphenyl-9H-fluorene (7.95 g, 20.0 mmol), tris(dibenzylideneacetone)dipalladium(0) (0.549 g, 0.600 mmol), BINAP (0.747 g, 1.20 mmol), sodium-t-butoxide (2.88 g, 30.0 mmol) and toluene (200 mL) was stirred at 100° C. for 3 h. The reaction solution was cooled to room temperature, and concentrated under reduced pressure. The resulting residue was purified by silica gel column chromatography to obtain Intermediate F as a white solid (7.44 g). The yield was 78%.

### Intermediate Synthesis Example 6: Synthesis of Intermediate G

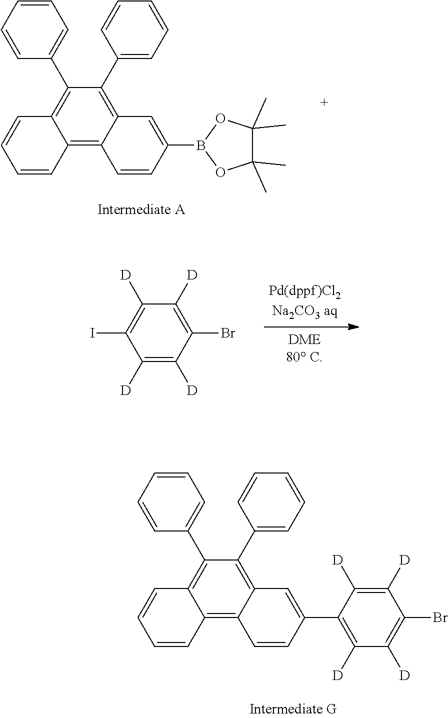 [SMILES: CC1(C)OB(c2ccc3c(c2)c(-c2ccccc2)c(-c2ccccc2)c2ccccc23)OC1(C)C ; [2H]c1c([2H])c(I)c([2H])c([2H])c1Br ; [2H]c1c([2H])c(-c2ccc3c(c2)c(-c2ccccc2)c(-c2ccccc2)c2ccccc23)c([2H])c([2H])c1Br]

Under an argon atmosphere, a mixture of Intermediate A (9.13 g, 20.0 mmol), 1-bromo-4-iodinebenzene-2,3,5,6-d4 (5.66 g, 20.0 mmol), [1,1′-bis(diphenylphosphino)ferrocene]dichloropalladium(II) (0.439 g, 0.600 mmol), sodium carbonate (6.36 g, 60.0 mmol), DME (100 mL) and water (30.0 mL) was stirred at 80° C. for 5 h. The reaction solution was cooled to room temperature, and concentrated under reduced pressure. The resulting residue was purified by silica gel column chromatography to obtain Intermediate G as a white solid (9.22 g). The yield was 95%.

### Synthesis Example 1: Synthesis of Compound Inv-1

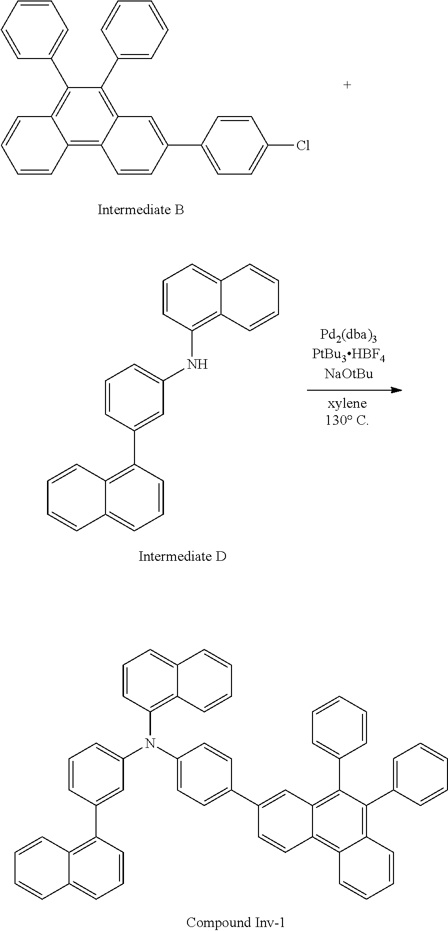 [SMILES: Clc1ccc(-c2ccc3c(c2)c(-c2ccccc2)c(-c2ccccc2)c2ccccc23)cc1 ; c1cc(Nc2cccc3ccccc23)cc(-c2cccc3ccccc23)c1 ; c1ccc(-c2c(-c3ccccc3)c3cc(-c4ccc(N(c5cccc(-c6cccc7ccccc67)c5)c5cccc6ccccc56)cc4)ccc3c3ccccc23)cc1]

Under an argon atmosphere, a mixture of Intermediate B (3.24 g, 7.36 mmol), Intermediate D (2.31 g, 6.69 mmol), tris(dibenzylideneacetone)dipalladium(0) (0.122 g, 0.134 mmol), tri-t-butylphosphonium tetrafluoroborate (0.155 g, 0.535 mmol), sodium-t-butoxide (0.900 g, 9.36 mmol), and xylene (67 mL) was stirred at 130° C. for 3 h. The reaction solution was cooled to room temperature, and concentrated under reduced pressure. The resulting residue was purified through silica gel column chromatography and recrystallization to obtain 3.32 g of white solid. The yield was 66%.

The resulting product was Compound 1 in the result of mass spectroscopy (m/e=750 for a molecular weight of 749.96).

### Synthesis Example 2: Synthesis of Compound Inv-2

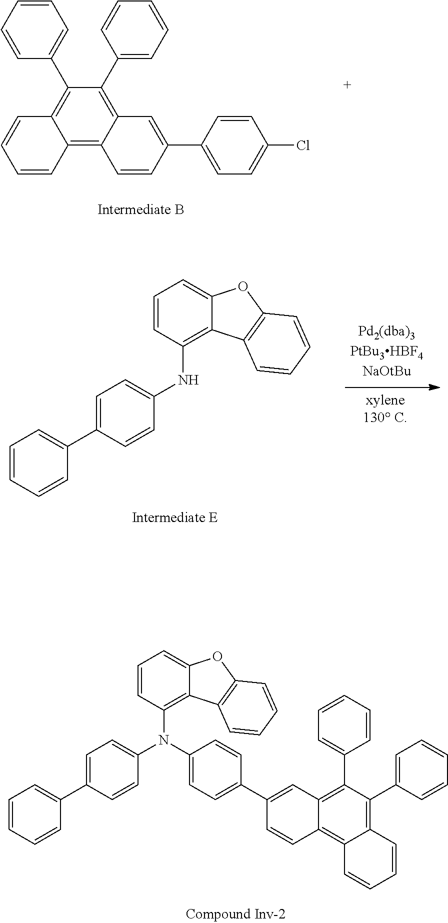 [SMILES: Clc1ccc(-c2ccc3c(c2)c(-c2ccccc2)c(-c2ccccc2)c2ccccc23)cc1 ; c1ccc(-c2ccc(Nc3cccc4oc5ccccc5c34)cc2)cc1 ; c1ccc(-c2ccc(N(c3ccc(-c4ccc5c(c4)c(-c4ccccc4)c(-c4ccccc4)c4ccccc45)cc3)c3cccc4oc5ccccc5c34)cc2)cc1]

A product was obtained by performing the same operation as in Synthesis Example 1 except that Intermediate E was used instead of Intermediate D used in Synthesis Example 1.

The resulting product was Compound Inv-2 in the result of mass spectroscopy (m/e=740 for a molecular weight of 739.92).

### Synthesis Example 3: Synthesis of Compound Inv-3

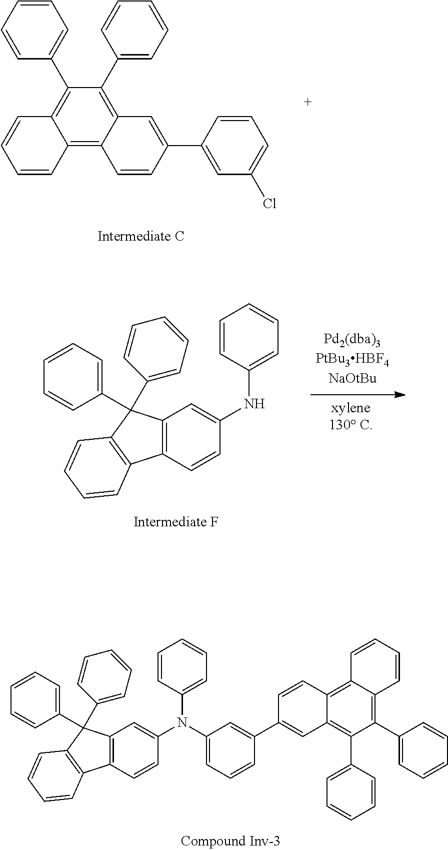 [SMILES: Clc1cccc(-c2ccc3c(c2)c(-c2ccccc2)c(-c2ccccc2)c2ccccc23)c1 ; c1ccc(Nc2ccc3c(c2)C(c2ccccc2)(c2ccccc2)c2ccccc2-3)cc1 ; c1ccc(-c2c(-c3ccccc3)c3cc(-c4cccc(N(c5ccccc5)c5ccc6c(c5)C(c5ccccc5)(c5ccccc5)c5ccccc5-6)c4)ccc3c3ccccc23)cc1]

Intermediate, Compound

A product was obtained by performing the same operation as in Synthesis Example 1 except that Intermediate F and Intermediate C were used instead of Intermediate D and Intermediate B used in Synthesis Example 1, respectively.

The resulting product was Compound Inv-3 in the result of mass spectroscopy (m/e=814 for a molecular weight of 814.04).

### Synthesis Example 4: Synthesis of Compound Inv-4

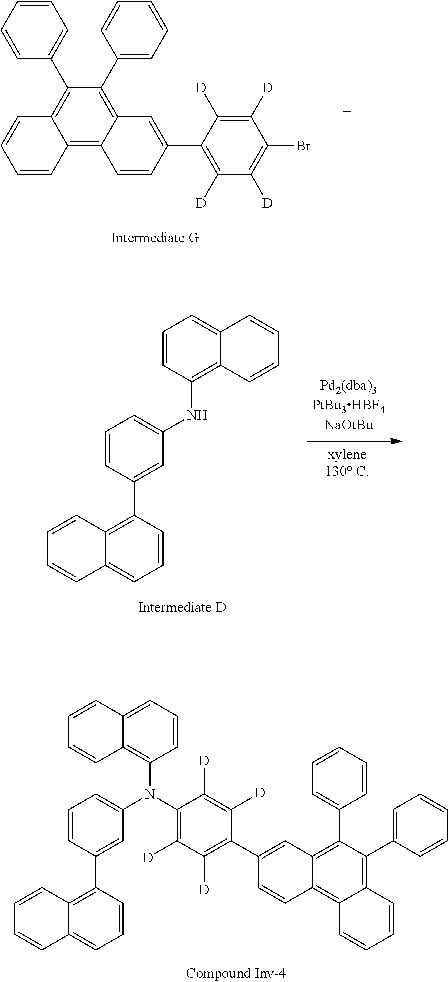 [SMILES: [2H]c1c([2H])c(-c2ccc3c(c2)c(-c2ccccc2)c(-c2ccccc2)c2ccccc23)c([2H])c([2H])c1Br ; c1cc(Nc2cccc3ccccc23)cc(-c2cccc3ccccc23)c1 ; [2H]c1c([2H])c(N(c2cccc(-c3cccc4ccccc34)c2)c2cccc3ccccc23)c([2H])c([2H])c1-c1ccc2c(c1)c(-c1ccccc1)c(-c1ccccc1)c1ccccc12]

A product was obtained by performing the same operation as in Synthesis Example 1 except that Intermediate G was used instead of Intermediate B used in Synthesis Example 1.

The resulting product was Compound Inv-4 in the result of mass spectroscopy (m/e=754 for a molecular weight of 753.98).

### REFERENCE SIGNS LIST

1, 11, 12: Organic EL device, 2: Substrate, 3: Anode, 4: Cathode, 5: Light emitting layer, 5a: First light emitting layer, 5b: Second light emitting layer, 6: Hole transporting zone (Hole transporting layer), 6a: Hole injecting layer, 6b: First hole transporting layer, 6c: Second hole transporting layer, 6d: Third hole transporting layer, 7: Electron transporting zone (Electron transporting layer), 7a: First electron transporting layer, 7b: Second electron transporting layer, 10, 20, 30: Light emitting unit

1. A compound represented by the following formula (1). 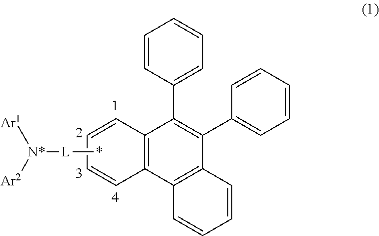
(in the formula,
N* is a central nitrogen atom;
* is bonded to any of carbon atoms 1 to 4;
L is a single bond, an unsubstituted phenylene group, or an unsubstituted biphenylene group.
Ar¹ is a group represented by the following formula (1a), (1b), (1c), (1d), (1e), or (1f); and
Ar² is a group represented by the following formula (1b), (1c), (1d), (1e), (1f), or (1g).) 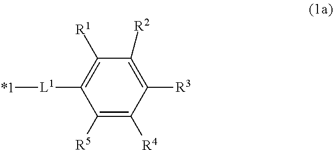
(in the formula,
each of R¹ to R⁵ is independently a hydrogen atom or a substituted or unsubstituted alkyl group having 1 to 6 carbon atoms;
adjacent two selected from R¹ to R⁵ are not bonded to each other, and thus do not form a ring;
L¹ is a single bond or an unsubstituted phenylene group; and
*1 is bonded to the central nitrogen atom N*.) 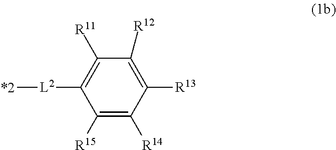
(in the formula,
each of R¹¹ to R¹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R¹¹ to R¹⁵ are not bonded to each other, and thus do not form a ring;
L² is an unsubstituted biphenylene group; and
*2 is bonded to the central nitrogen atom N*.) 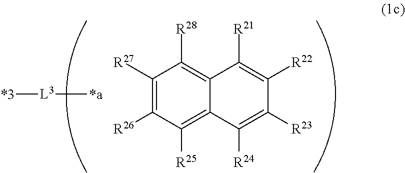
(in the formula,
each of R²¹ to R²⁸ is independently a hydrogen atom, a substituted or unsubstituted alkyl group having 1 to 6 carbon atoms, or a substituted or unsubstituted aryl group having 6 to 12 ring carbon atoms;
provided that one selected from R²¹ to R²⁸ is a single bond bonded to *a, and adjacent two selected from R²¹ to R²⁸ except for the single bond are not bonded to each other, and thus do not form a ring structure;
L³ is a single bond or an unsubstituted arylene group having 6 to 12 ring carbon atoms; and
*3 is bonded to the central nitrogen atom N*.) 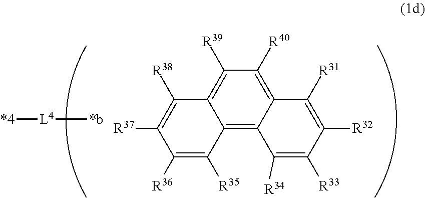
(in the formula,
each of R³¹ to R⁴⁰ is independently a hydrogen atom, a substituted or unsubstituted alkyl group having 1 to 6 carbon atoms, or a substituted or unsubstituted aryl group having 6 to 12 ring carbon atoms;
provided that one selected from R³¹ to R⁴⁰ is a single bond bonded to *b, and adjacent two selected from R³¹ to R⁴⁰ except for the single bond are not bonded to each other, and thus do not form a ring structure;
L⁴ is a single bond or an unsubstituted arylene group having 6 to 12 ring carbon atoms; and
*4 is bonded to the central nitrogen atom N*.) 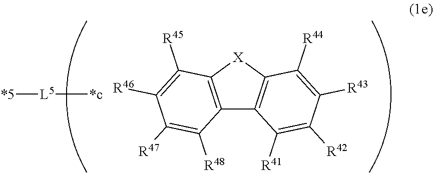
(in the formula,
X is an oxygen atom, a sulfur atom, NRa, or CRbRc;
Ra is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
R bis a substituted or unsubstituted aryl group having 6 to 12 ring carbon atoms;
Rc is a substituted or unsubstituted alkyl group having 1 to 6 carbon atoms or a substituted or unsubstituted aryl group having 6 to 12 ring carbon atoms;
Rb and Rc may be bonded to each other to form a spiro ring structure;
each of R⁴¹ to R⁴⁸ is independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
provided that one selected from R⁴¹ to R⁴⁸ and Ra is a single bond bonded to *c;
adjacent two selected from R⁴¹ to R⁴⁸ except for the single bond may be bonded to each other to form one or more substituted or unsubstituted benzene rings. Meanwhile, when X is CRbRc, the adjacent two do not form a ring;
L⁵ is a single bond or an unsubstituted arylene group having 6 to 12 ring carbon atoms; and
*5 is bonded to the central nitrogen atom N*.) 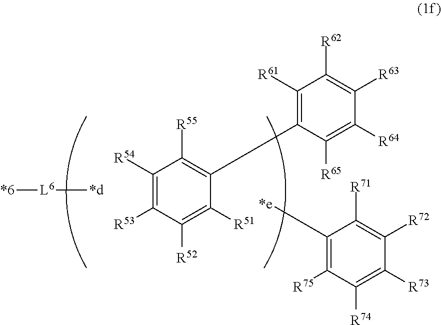
(in the formula,
each of R⁵¹ to R⁵⁵ is independently a hydrogen atom, an unsubstituted alkyl group having 1 to 6 carbon atoms, or an unsubstituted phenyl group;
provided that one selected from R⁵¹ to R⁵⁵ is a single bond bonded to *d, and another selected from R⁵¹ to R⁵⁵ is a single bond bonded to *e;
except for the single bond bonded to *d and the single bond bonded to *e, adjacent two selected from R⁵¹ to R⁵⁵ are not bonded to each other, and thus do not form a ring structure;
each of R⁶¹ to R⁶⁵ and R⁷¹ to R⁷⁵ is independently a hydrogen atom or an unsubstituted alkyl group having 1 to 6 carbon atoms;
adjacent two selected from R⁶¹ to R⁶⁵ and R⁷¹ to R⁷⁵ may be bonded to each other to form one or more substituted or unsubstituted benzene rings;
L⁶ is a single bond or an unsubstituted arylene group having 6 to 12 ring carbon atoms; and
*6 is bonded to the central nitrogen atom N*.) 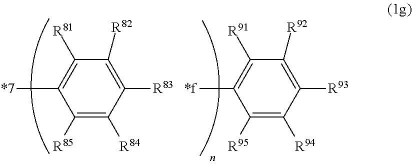
(in the formula,
each of R⁸¹ to R⁸⁵ is independently a hydrogen atom or a substituted or unsubstituted alkyl group having 1 to 6 carbon atoms;
provided that one selected from R⁸¹, R⁸², R⁸⁴, and R⁸⁵ is a single bond bonded to *f;
adjacent two selected from R⁸³ and R⁸¹, R⁸², R⁸⁴, and R⁸⁵ except for the single bond bonded to *f are not bonded to each other, and thus do not form a ring structure;
each of R⁹¹ to R⁹⁵ is independently a hydrogen atom, a substituted or unsubstituted alkyl group having 1 to 6 carbon atoms, or a substituted or unsubstituted aryl group having 6 to 12 ring carbon atoms;
adjacent two selected from R⁹¹ to R⁹⁵ are not bonded to each other, and thus do not form a ring structure;
n is 0 or 1, and then when n is 0, the formula (1g) indicates the following formula (1g′); and 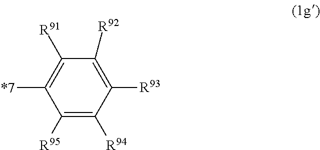
*7 in the formulas (1g) and (1f) is bonded to the central nitrogen atom.)

2. The compound according to claim 1, wherein * is bonded to carbon atom 1 or carbon atom 2.

3. The compound according to claim 1, wherein L is a single bond.

4. The compound according to claim 1, wherein L is an unsubstituted phenylene group.

5. The compound according to claim 1, wherein Li in the formula (1a), L³ in the formula (1c), L⁴ in the formula (1d), L⁵ in the formula (1e), and L⁶ in the formula (1f) are unsubstituted phenylene groups.

6. The compound according to claim 1, wherein Ar¹ is a group represented by the formula (1a), (1c), (1d), or (1f).

7. The compound according to claim 1, wherein Ar¹ is a group represented by the formula (1a), (1c), (1d), or (1f), and Ar² is a group represented by (1b), (1c), (1d), (1e), (1f), or (1g).

8. The compound according to claim 1, wherein Ar¹ is a group represented by the formula (1a), (1c), (1d), or (1f), and Ar² is a group represented by (1c), (1d), (1e), (1f), or (1g).

9. The compound according to claim 1, wherein Ar¹ is a group represented by the formula (1a), (1c), (1d), or (1f), and Ar² is a group represented by (1c), (1e), (1f), or (1g).

10. The compound according to claim 1, wherein all of R¹ to R⁵ in the formula (1a) are hydrogen atoms.

11. The compound according to claim 1, wherein all of R¹¹ to R¹⁵ in the formula (1b) are hydrogen atoms.

12. The compound according to claim 1, wherein all of R²¹ to R²⁸ except for the single bond bonded to *a in the formula (1c) are hydrogen atoms.

13. The compound according to claim 1, wherein all of R³¹ to R⁴⁰ except for the single bond bonded to *b in the formula (1d) are hydrogen atoms.

14. The compound according to claim 1, wherein all of R⁴¹ to R⁴⁸ except for the single bond bonded to *c in the formula (1e) are hydrogen atoms.

15. The compound according to claim 1, wherein in the formula (1f), except for the single bond bonded to *d and the single bond bonded to *e, all of R⁵¹ to R⁵⁵ are hydrogen atoms, and all of R⁸¹ to R⁶⁵ and R⁷¹ to R⁷⁵ are hydrogen atoms.

16. The compound according to claim 1, wherein in the formula (1g), R⁸³ and all of R⁸¹, R⁸², R⁸⁴, and R⁸⁵ except for the single bond bonded to *f are hydrogen atoms, and all of R⁸¹ to R⁹⁵ are hydrogen atoms.

17. The compound according to claim 1, which contains at least one deuterium atom.

18. A material for an organic electroluminescence device, which contains the compound according to claim 1.

19. A hole transporting layer material containing the compound according to claim 1.

20. An organic electroluminescence device comprising a cathode, an anode, and organic layers between the cathode and the anode,
wherein the organic layers include a light emitting layer, and at least one layer of the organic layers contains the compound according to claim 1.

21. The organic electroluminescence device according to claim 20, wherein the organic layers include a hole transporting zone between the anode and the light emitting layer, and the hole transporting zone contains the compound.

22. The organic electroluminescence device according to claim 21, wherein the hole transporting zone includes a first hole transporting layer on the anode side and a second hole transporting layer on the cathode side, and either or both of the first hole transporting layer and the second hole transporting layer contains the compound.

23. The organic electroluminescence device according to claim 22, wherein the second hole transporting layer contains the compound.

24. The organic electroluminescence device according to claim 22, wherein the light emitting layer and the second hole transporting layer are in direct contact with each other.

25. The organic electroluminescence device according to claim 22, wherein a sum of a thickness of the first hole transporting layer and a thickness of the second hole transporting layer is 30 nm or more, and 150 nm or less.

26. The organic electroluminescence device according to claim 20, wherein the light emitting layer is a single layer.

27. The organic electroluminescence device according to claim 20, wherein the light emitting layer contains a light-emitting compound that exhibits fluorescence emission with a main peak wavelength of 500 nm or less.

28. The organic electroluminescence device according to claim 20, wherein the light emitting layer contains a fluorescent dopant material.

29. An electronic device including the organic electroluminescence device according to claim 20.
